(19) United States
(12) Patent Application Publication
(10) Pub. No.: US 2023/0075314 A1
(43) Pub. Date: Mar. 9, 2023

(54) VECTORIZED ANTIBODIES (vAb) AND USES THEREOF

(71) Applicant: Voyager Therapeutics, Inc., Cambridge, MA (US)

(72) Inventors: Jinzhao Hou, Cambridge, MA (US); Yanqun Shu, Cambridge, MA (US); Todd Carter, Cambridge, MA (US); Dinah Wen-Yee Sah, Hopkinton, MA (US); Po-Jen Yen, Cambridge, MA (US); Donna T. Ward, Groton, MA (US); Johanna L. Crimins, Groton, MA (US)

(21) Appl. No.: 17/607,213

(22) PCT Filed: Apr. 29, 2020
(86) PCT No.: PCT/US2020/030360

§ 371 Date: Oct. 28, 2021

Related U.S. Application Data
(60) Provisional application No. 62/839,891, filed on Apr. 29, 2019.
(60) Provisional application No. 62/860,295, filed on Jun. 12, 2019.
(60) Provisional application No. 62/926,706, filed on Oct. 28, 2019.
(60) Provisional application No. 63/002,008, filed on Mar. 30, 2020.
(60) Provisional application No. 63/002,011, filed on Mar. 30, 2020.

(51) Int. Cl. C12N 15/86; C12N 15/113; C07K 16/00

(52) U.S. Cl. CPC C12N 15/86; C12N 15/113; C07K 16/00; C12N 2750/14143; C12N 2830/50; C07K 2317/24; A61K 2039/505; C12N 2310/14; C12N 2310/141; C07K 2319/095; C07K 2317/14; C12N 2750/14171; C07K 2317/31

(57) ABSTRACT

The present disclosure provides compositions and methods for the preparation, manufacture and therapeutic use of viral vectors, such as adeno-associated virus (AAV) particles having viral genomes encoding one or more antibodies or antibody fragments or antibody-like polypeptides, for the prevention and/or treatment of diseases and/or disorders.

### REFERENCE TO RELEVANT APPLICATIONS

This application claims the benefit of U.S. Provisional Patent Application No. 62/839,891, entitled “Compositions and Methods for the Treatment of Tauopathy”, filed Apr. 29, 2019, U.S. Provisional Patent Application No. 62/860,295, filed Jun. 12, 2019, entitled “Vectorized Antibodies (vAb) and Uses Thereof”, U.S. Provisional Patent Application No. 62/926,706, filed Oct. 28, 2019, entitled “Vectorized Antibodies (vAb) and Uses Thereof”, U.S. Provisional Patent Application No. 63/002,008, filed Mar. 30, 2020, entitled “Vectorized Antibodies (vAb) and Uses Thereof”, U.S. Provisional Patent Application No. 63/002,011, filed Mar. 30, 2020, entitled “Compositions and Methods for the Treatment of Tauopathy”; the contents of each of which are herein incorporated by reference in their entirety.

### SEQUENCE LISTING

The present application is being filed along with a Sequence Listing in electronic format. The Sequence Listing file, entitled 2057_1311PCT_SL, was created on Apr. 29, 2020, and is 27,502,408 bytes in size. The information in electronic format of the Sequence Listing is incorporated herein by reference in its entirety.

### FIELD OF THE DISCLOSURE

This disclosure relates to compositions of vectorized antibodies and methods for vectored antibody delivery (VAD).

### BACKGROUND OF THE DISCLOSURE

Antibody-based therapies have been developed for a wide variety of diseases, disorders and conditions, including infectious and non-infectious diseases. The U.S. Food and Drug Administration (FDA) has approved antibodies for treatment of cancers, autoimmune and immune system disorders, ocular diseases, nervous system diseases, inflammations, and infections, amongst many others. Naturally, antibodies are components of the adaptive immune response and they function by recognizing specific foreign antigens and stimulating humoral immunity responses. As a consequence, antibodies may be applied to the treatment, prevention, management, diagnosis and research of diseases, disorders and/or conditions.

Antibodies have relatively short half-lives and this presents an ongoing and long-felt challenge for antibody-based therapies. In order to achieve a sufficiently high concentration of an antibody for long lasting therapeutic effects, antibody therapies are traditionally delivered by repeated administration, e.g. by multiple injections. This dosing regimen results in an inconsistent level of antibody throughout the treatment period, limited efficiency per administration, high cost of administration and consumption of the antibody. Hence, there remains a need in the art for delivery of antibodies and antibody-based therapeutics through alternative routes or modalities of administration.

One such alternative route of administration is by expression vector (e.g. plasmid or viral vector), including but not limited to, adeno-associated viral vectors (AAVs). Adeno-associated viral vectors are widely used in gene therapy approaches due to a number of advantageous features. As dependoparvoviruses, AAV are non-replicating in infected cells and therefore not associated with any known disease. Further, AAVs may be introduced to a wide variety of host cells, do not integrate into the genome of the host cell, and are capable of infecting both quiescent and dividing cells. AAVs transduce non-replicating and long-lived cells in vivo, resulting in long term expression of the protein of interest. Further, AAVs can be manipulated with cellular and molecular biology techniques to produce non-toxic particles carrying a payload encoded in the AAV viral genome that can be delivered to a target tissue or set of cells with limited or no side-effects. Given the foregoing, the use of AAVs for vectored antibody delivery (VAD) would allow for longer lasting efficacy, fewer dose treatments, and more consistent levels of the antibody throughout the treatment period.

In vectored antibody delivery (VAD) an V is used as the delivery modality for a nucleic acid sequence encoding the antibody, which results in in vivo expression of the encoded payload, e.g., functional antibody.

The mechanism underlying VAD is thought to proceed through the following steps. First the AAV vector enters the cell via endocytosis, then escapes from the endosomal compartment and is transported to the nucleus wherein the viral genome is released and converted into a double-stranded episomal molecule of DNA by the host. The transcriptionally active episome results in the expression of encoded antibodies that may then be secreted from the cell into the circulation. VAD may therefore enable continuous, sustained and long-term delivery of antibodies administered by a single injection of an AAV particle.

Previous studies of an AAV-mediated antibody technique known as vectored immunoprophylaxis (VIP) have focused on neutralization of human immunodeficiency virus (HIV) (see, e.g. Johnson et al., 2009, Nature Med., 15, 901-906, Saunders et al., 2015, J. Virol., 89(16), 8334-8345, Balasz et al., 2012, Nature 481, 81-84, the contents of which are incorporated herein by reference in their entirety). Balasz et al. reported along-term, even lifelong, expression of monoclonal antibody at high concentration from a single intramuscular administration in mice that resulted in full protection against HIV infection. AAV-mediated VIP has also been demonstrated against influenza strains (see, e.g. Balasz, et al. Nat. Biotechnol., 2013, 31(7):647-52) and Plasmodium Falciparum, a sporozoite causing malaria infection (see, e.g. Deal at al., 2014, PNAS, 111 (34), 12528-12532), as well as cancer, RSV and drug addiction (see, e.g. review by Schnepp and Johnson, Microbiol. Spectrum 2(4), 2014). Though promising, these studies emphasize efforts to merely prevent disease. There still remains a need for improved methods of prevention, and new antibody-mediated therapies for research, diagnosis, and treatment of disease.

The present disclosure addresses this need by providing novel AAV particles having viral genomes engineered to encode antibodies and antibody-based compositions and methods of using these constructs (e.g., VAD) for the treatment, prevention, diagnosis and research of diseases, disorders and/or conditions. The present disclosure further embraces optimized AAV particles for delivery of nucleic acids (e.g., viral genomes) encoding antibodies and antibody-based compositions to a subject in need thereof.

### SUMMARY OF THE DISCLOSURE

The disclosure provides AAV particles comprising a capsid and a viral genome, said viral genome comprising a 5′ inverted terminal repeat (ITR) sequence region, at least one promoter sequence region, a polyA sequence region, a 3′ITR sequence region, and at least one payload region comprising a first nucleic acid sequence encoding an antibody, an antibody fragment or an antibody variant, wherein the 5′ITR sequence region may be, but is not limited to, SEQ ID NO: 13519 or 13520, wherein the 3′ITR sequence region may be, but is not limited to, SEQ ID NO: 13521 or 13522, wherein the at least one promoter sequence region may be, but is not limited to, one or more of SEQ ID NO: 13523-13534, and wherein the polyA sequence region may be, but is not limited to, SEQ ID NO: 13576, 13577, or 13578.

In one aspect, the viral genome comprises an 5′ITR sequence region may be, but is not limited to, SEQ ID NO: 13519, an 3′ITR sequence region may be, but is not limited to, SEQ ID NO: 13521, and a polyA sequence region may be, but is not limited to, SEQ ID NO: 13576. In one aspect, the viral genome comprises an 5′ITR sequence region may be, but is not limited to, SEQ ID NO: 13519, an 3′ITR sequence region may be, but is not limited to, SEQ ID NO: 13521, and a polyA sequence region may be, but is not limited to, SEQ ID NO: 13577. In one aspect, the viral genome comprises an 5′ITR sequence region may be, but is not limited to, SEQ ID NO: 13520, an 3′ITR sequence region may be, but is not limited to, SEQ ID NO: 13522, and a polyA sequence region may be, but is not limited to, SEQ ID NO: 13576. In one aspect, the viral genome comprises an 5′ITR sequence region may be, but is not limited to, SEQ ID NO: 13520, the 3′ITR sequence region may be, but is not limited to, SEQ ID NO: 13522, and a polyA sequence region may be, but is not limited to, SEQ ID NO: 13577.

In one aspect, the viral genome comprises at least one promoter sequence. The promoter sequence region may be, but is not limited to, SEQ ID NO: 13523, 13524, 13525, 13526, 13527, 13528, 13529, 13530, 13531, 13532, 13533, and/or 13534. In one aspect, the viral genome comprises at least two promoters which may be, but is not limited to, SEQ ID NO: 13524 and 13525.

In one aspect, the viral genome comprises at least one intron sequence region. The intron sequence region may independently be, but is not limited to, SEQ ID NO: 13540-13554. In one aspect, the viral genome also includes at least one exon region which may be, but is not limited to, SEQ ID NO: 13535-13539. In one aspect, the viral genome comprises two intron sequence regions and two exon sequence regions.

In one aspect, the viral genome comprises a filler sequence region. The filler sequence region may be, but is not limited to, SEQ ID NO: 13579 or 13580.

In one aspect, the viral genome comprises a tag sequence region. The tag sequence region may be, but is not limited to, SEQ ID NO: 13571-13575.

In one aspect, the viral genome comprises at least one signal sequence region. The signal sequence region may be, but is not limited to, SEQ ID NO: 13555-13570.

The disclosure also provides AAV particles comprising a capsid and a viral genome, said viral genome comprising at least one inverted terminal repeat (ITR) region and a payload region, said payload region comprising a regulatory sequence operably linked to at least a first nucleic acid segment, said first nucleic acid segment encoding one or more polypeptides given in Table 3-16, variants and fragments thereof. The capsid of the AAV particle may be any of the serotypes described herein and/or described in Table 1.

In one aspect, the first nucleic acid segment may encode one or more polypeptides such as, but not limited to, an antibody heavy chain, an antibody light chain, a linker, and combinations thereof. The first nucleic acid segment may encode one or more polypeptides which is humanized. As a non-limiting example, the first nucleic acid segment encodes from 5′ to 3′, an antibody heavy chain, a linker, and an antibody light chain. As another non-limiting example, the first nucleic acid segment encodes from 5′ to 3, an antibody light chain, a linker, and an antibody heavy chain. As yet another non-limiting example, the first nucleic acid segment encodes one or more antibody heavy chains. As yet another non-limiting example, the first nucleic acid segment encodes one or more antibody light chains.

In one aspect, the first nucleic acid segment includes an antibody, having at least 95% identity to any of the sequences of Table 3-16, including, SEQ ID NO. 1740-10916 and 13165-13518.

In one aspect, the first nucleic acid segment encodes an antibody, having at least 95% identity to any of the sequences of Table 3-16, including, SEQ ID NO: 1740-10916 and 13165-13518.

In one aspect, the regulatory sequence may comprise a promoter such as, but not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

In one aspect, the linker in the viral genome is selected from one or more of the linkers given in Table 2.

In one aspect, the AAV particles described herein may comprise a viral genome which is single stranded.

In one aspect, the AAV particles described herein may comprise a viral genome which is self-complementary.

In one aspect, the AAV particles described herein may comprise a viral genome comprising at least one intron sequence.

In one aspect, the AAV particles described herein may comprise a viral genome comprising at least one stuffer sequence to adjust the length of the viral genome to increase efficacy and/or efficiency.

In one aspect, the AAV particles described herein may comprise at least one region which has been codon optimized. As a non-limiting example, the viral genome may be codon optimized. As another non-limiting example, the first nucleic acid segment is codon-optimized.

In one aspect, the AAV particles described herein may comprise a viral genome with two ITR regions. At least one of the ITR regions may be derived from the same or different parental serotype of the capsid. As a non-limiting example, at least one ITR region is derived from AAV2.

In one aspect, the AAV particles comprise a viral genome which comprises a second nucleic acid segment. The second nucleic acid segment may encode an aptamer, siRNA, saRNA, ribozyme, microRNA, mRNA or combination thereof.

In one aspect, the AAV particles comprise a viral genome which comprises a second nucleic acid segment encoding an siRNA designed to target the mRNA that encodes the target of the antibody encoded by the first nucleic acid segment.

In one aspect, the AAV particles comprise a viral genome which comprises a second nucleic acid segment encoding a microRNA, the microRNA is selected to target the mRNA that encodes the target of the antibody encoded by the first nucleic acid segment.

In one aspect, the AAV particles comprise a viral genome which comprises a second nucleic acid segment encoding an mRNA, the mRNA encodes one or more peptides inhibitors of the same target of the antibody encoded by the first nucleic acid segment.

In one aspect, the AAV particles comprise a viral genome which comprises a third nucleic acid segment. The third nucleic acid segment may encode a nuclear export signal, a polynucleotide or polypeptide which acts as a regulator of expression of the viral genome in which it is encoded, a polynucleotide or polypeptide which acts as a regulator of expression of the payload region of the viral genome in which it is encoded and/or a polynucleotide or polypeptide which acts as a regulator of expression of the first nucleic acid segment of the payload region of the viral genome in which it is encoded.

The disclosure provides AAV particles comprising a capsid and a viral genome, said viral genome comprising at least one inverted terminal repeat (ITR) region and a payload region comprising a regulatory sequence operably linked to at least a first nucleic acid segment, the first nucleic acid segment encoding a bispecific antibody derived from any of the sequences listed in Table 3-16 or portions or fragments thereof.

The disclosure provides methods of producing a functional antibody in a subject in need thereof, comprising administering to a subject the AAV particles described herein. The level or amount of the functional antibody in the target cell or tissue after administration to the subject may be from about 0.001 ug/mL to 100 mg/mL. The functional antibody may be encoded by a single first nucleic acid segment of a viral genome within the AAV particle. The functional antibody may be encoded by two different viral genomes, the two different viral genomes may be packaged in separate capsids.

The disclosure provides a pharmaceutical composition comprising an AAV particle described herein in a pharmaceutically acceptable excipient. As a non-limiting example, the pharmaceutically acceptable excipient is saline. As a non-limiting example, the pharmaceutically acceptable excipient is 0.001% pluronic in saline.

The disclosure provides methods of producing a functional antibody in a subject in need thereof, comprising administering to a subject the AAV particles described herein by a delivery route such as, but not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the SUBSTITUTE SHEET (RULE 26) gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

The disclosure provides methods of treating and/or preventing a disease or disorder in a subject comprising administering to the subject an AAV particle described herein. The administration may be at a prophylactically effective dose such as, but not limited to, from about 1 ug/mL to about 500 ug/mL of expressed polypeptide or 1x10e4 to 1x10e16 VG/mL from the pharmaceutical composition. The pharmaceutical composition may be administered at least once. The pharmaceutical composition may be administered daily, weekly, monthly or yearly. The pharmaceutical composition may be co-administered as part of a combination therapy.

### DETAILED DESCRIPTION

### I. Compositions of the Disclosure

According to the present disclosure, compositions for delivering functional anti-tau antibodies and/or antibody-based compositions by adeno-associated viruses (AAVs) are provided. AAV particles may be provided via any of several routes of administration, to a cell, tissue, organ, or organism, in vivo, ex vivo, or in vitro.

As used herein, an “AAV particle” is a virus which comprises a viral genome with at least one payload region and at least one inverted terminal repeat (ITR) region.

As used herein, “viral genome” or “vector genome” refers to the nucleic acid sequence(s) encapsulated in an V particle. Viral genomes comprise at least one payload region encoding polypeptides, e.g., antibodies, antibody-based compositions or fragments thereof.

As used herein, a “payload” or “payload region” is any nucleic acid molecule which encodes one or more polypeptides. At a minimum, a payload region comprises nucleic acid sequences that encode an antibody, an antibody-based composition, or a fragment thereof, but may also optionally comprise one or more functional or regulatory elements to facilitate transcriptional expression and/or polypeptide translation.

As used herein, “VL” and “VH” refer to components of alight chain or heavy chain of an antibody, respectively, or a fragment thereof. In some embodiments “VL” and “VH” refer to the variable regions of the light or heavy chain of an antibody, respectively, or a fragment thereof. In another embodiment, “VL” and “VH” may also embrace a constant region of alight or heavy chain of an antibody, or a fragment thereof. In another embodiment, “VL” and “VH” may embrace the entirety of an antibody light chain or heavy chain, respectively.

In some embodiments, AAV particles, viral genomes and/or payloads, and the methods of their use may be as described in WO2017189963, the contents of which are herein incorporated by reference in their entirety.

The nucleic acid sequences and polypeptides disclosed herein may be engineered to contain modular elements and/or sequence motifs assembled to enable expression of the antibodies or antibody-based compositions. In some embodiments, the nucleic acid sequence comprising the payload region may comprise one or more of a promoter region, an intron, a Kozak sequence, an enhancer, or a polyadenylation sequence. Payload regions typically encode antibodies or antibody-based compositions, which may include an antibody heavy chain domain, an antibody light chain domain, both antibody heavy and light chain domains, or fragments of the foregoing in combination with each other or in combination with other polypeptide moieties. In some cases, payload regions may also encode one or more linkers or joining regions between antibody heavy and light chain domains or fragments. The order of expression, structural position, or concatemer count (heavy chain, light chain, or linker) may be different within or among different payload regions. The identity, position and number of linkers expressed by payload regions may also vary.

The payload regions may be delivered to one or more target cells, tissues, organs, or organisms within the viral genome of an AAV particle.

### Adeno-Associated Viruses (AAVs) and AAV Particles

Adeno-associated viruses (MV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203.736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV219).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, AAV particles of the present disclosure are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In some embodiments, the viral genome of the AAV particles of the present disclosure comprise at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the transduced cell.

In some embodiments, the AAV particle of the present disclosure is an scAAV.

In some embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments, the capsids of the AAV particles are engineered according to the methods described in US Publication Number US20130195801, the contents of which are incorporated herein by reference in their entirety.

In some embodiments, the AAV particles comprising a payload region encoding the polypeptides may be introduced into mammalian cells.

### AAV Serotypes

AAV particles of the present disclosure may comprise or be derived from any natural or recombinant AAV serotype. According to the present disclosure, the AAV particles may utilize or be based on a serotype or include a peptide selected from any of the following VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP (3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9 K449R, AAV9.11, AAV9.13, AAV9.16, AAV9.24, AAV9.45, AAV9.47, AAV9.61, AAV9.68, AAV9.84, AAV9.9, AAV10, AAV11, AAV2, AAV6.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42.10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-81rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8hu.10, AAV16.12/hu.11, AAV29.3/bb.1, AAV29.5/bb.2, AAV106.1/hu.37, AAV114.31hu.40, AAV127.2hu.41, AAV127.5/hu.42, AAV128.3/hu.44, AAV130.41hu.48, AAV145.1hu.53, AAV145.5/hu.54, AAV145.6/hu.55, AAV161.101hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER114, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK12, AAV-LK92, AAV-LK03, AAV-LK04, AAV-LK05, AAV-LK06, AAV-LK97, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-75, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv1, AAV CLv1-1, AAV CLv1-10, AAV CLv1-2, AAV CLv12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the AAV serotype may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-1b (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42. 5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV serotype may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID N: 1 of US20150159173), rh32133 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID N: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV serotype may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 Is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gin) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.11hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.11hu.6 (SEQ ID NO: 5 and 84 of WO2005033321), AAV3.11hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.41hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151, 154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV serotype may be, or have, a sequence as described in U.S. Pat. No. 9,233,131, the contents of which are herein incorporated by reference in their entirety, such as, but not limited to, AAVhE1.1 SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV serotype may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID N0:16 of US20150376607), AAV-LK16 (SEQ ID N:17 of US20150376607), AAV-LK17 (SEQ ID N:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID N:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 US9163261), or variants thereof.

In some embodiments, the AAV serotype may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: of US20150376240), AAV-h (SEQ ID NO: 2 of US20150376240), AAV-b (SEQ ID NO: 1 of US20150376240), or variants thereof.

In some embodiments, the AAV serotype may be, or have, a sequence as described in United States Patent Publication No. US20160017295, the contents of which are herein incorporated by reference in their entirety, such as, but not limited to, AAV SM 102 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10.6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 101 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV serotype may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV serotype may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV5421hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.21hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use may be from a variety of species. In some embodiments, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In some embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F4175), AAV9.5 (A1235G, A1314T, A1642G, C1760T; Q412R, T548A, A587V), AAV9.6 (T1231A; F4111), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T4505), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, 5469R, A555V, G604V), AAV9.54 (C1531A, T1609A; L5111, L537M), AAV9.55 (T1605A; F535L), AAV9.58 (C1475T, C1579A; T4921, H527N), AAV.59 (T1336C; Y446H), AAV9.61 (A1493T; N4981), AAV9.64 (C1531A, A1617T; 15111), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV serotype may be, or have, a sequence as described in International Publication No. WO2016049230, the contents of which are herein incorporated by reference in their entirety, such as, but not limited to AAVF1/HSC1 (SEQ ID NO: 2 and 20 of WO2016049230), AAVF2/HSC2 (SEQ ID NO: 3 and 21 of WO2016049230), AAVF3/HSC3 (SEQ ID NO: 5 and 22 of WO2016049230), AAVF4/HSC4 (SEQ ID NO: 6 and 23 of WO2016049230), AAVF5/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), AAVF121HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF171HSC17 (SEQ ID NO: 13 and 35 of WO2016049230), or variants or derivatives thereof.

In some embodiments, the V serotype may be, or have, a sequence as described in U.S. Pat. No. 8,734,809, the contents of which are herein incorporated by reference in their entirety, such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), V CBr-E3 (SEQ ID NO: 15 and 89 of U.S. Pat. No. 8,734,809), V CBr-E4 (SEQ ID NO: 16 and 90 of U.S. Pat. No. 8,734,809), V CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U88734809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U88734809), MV CLv-D4 (SEQ ID NO: 25 and 99 of U.S. Pat. No. 8,734,809), AAV CLv-D5 (SEQ ID NO: 26 and 100 of U.S. Pat. No. 8,734,809), AAV CLv-D (SEQ ID NO: 27 and 101 of U.S. Pat. No. 8,734,809), V CLv. D7 (SEQ ID NO: 28 and 102 of U.S. Pat. No. 8,734,809), V CLv-D8 (SEQ ID NO: 29 and 103 of U.S. Pat. No. 8,734,809), V CLv-E1 (SEQ ID NO: 13 and 87 of U.S. Pat. No. 8,734,809), AAV CLv-R1 (SEQ ID NO: 30 and 104 of U88734809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), V CLv-R4 (SEQ ID NO: 33 and 107 of U88734809), AAV CLv-R5 (SEQ ID NO: 34 and 108 of U88734809), V CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R (SEQ ID NO: X and X of U88734809), AAV CLv-R9 (SEQ ID NO: X and X of U.S. Pat. No. 8,734,809), AAV CLg-F1 (SEQ ID NO: 39 and 113 of U88734809), V CLg-F2 (SEQ ID NO: 40 and 114 of U.S. Pat. No. 8,734,809), V CLg-F3 (SEQ ID NO: 41 and 115 of U.S. Pat. No. 8,734,809), AAV CLg-F4 (SEQ ID NO: 42 and 116 of U88734809), AAV CLg-F5 (SEQ ID NO: 43 and 117 of U.S. Pat. No. 8,734,809), V CLg-F6 (SEQ ID NO: 43 and 117 of U.S. Pat. No. 8,734,809), AAV CLg-F7 (SEQ ID NO: 44 and 118 of U.S. Pat. No. 8,734,809), V CLg-F8 (SEQ ID NO: 43 and 117 of U.S. Pat. No. 8,734,809), V CSp. (SEQ ID NO: 45 and 119 of U88734809), V CSp-10 (SEQ ID NO: 46 and 120 of U88734809), V CSp-11 (SEQ ID NO: 47 and 121 of U88734809), AAV CSp-2 (SEQ ID NO: 48 and 122 of U88734809), AAV CSp-3 (SEQ ID NO: 49 and 123 of U.S. Pat. No. 8,734,809), AAV CSp-4 (SEQ ID NO: 50 and 124 of U88734809), AAV CSp-6 (SEQ ID NO: 51 and 125 of U88734809), AAV CSp-7 (SEQ ID NO: 52 and 126 of U.S. Pat. No. 8,734,809), MV CSp-8 (SEQ ID NO: 53 and 127 of U88734809), MV CSp-9 (SEQ ID NO: 54 and 128 of U.S. Pat. No. 8,734,809), AAV CHt-2 (SEQ ID NO: 55 and 129 of U88734809), MV CHt-3 (SEQ ID NO: 56 and 130 of U88734809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), V CKd-2 (SEQ ID NO: 59 and 133 of U.S. Pat. No. 8,734,809), AAV CKd-3 (SEQ ID NO: 60 and 134 of U88734809), AAV CKd-4 (SEQ ID NO: 61 and 135 of U88734809), AAV CKd-6 (SEQ ID NO: 62 and 136 of U88734809), V CKd-7 (SEQ ID NO: 63 and 137 of U88734809), MV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U88734809), AAV CLv13 (SEQ ID NO: 67 and 141 of U.S. Pat. No. 8,734,809), AAV CLv-2 (SEQ ID NO: 68 and 142 of U88734809), AAV CLv-3 (SEQ ID NO: 69 and 143 of U.S. Pat. No. 8,734,809), AAV CLv-4 (SEQ ID NO: 70 and 144 of U.S. Pat. No. 8,734,809), AAV CLv-6 (SEQ ID NO: 71 and 145 of U88734809), AAV CLv-8 (SEQ ID NO: 72 and 146 of U88734809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U88734809), AAV CKd-B6 (SEQ ID NO: 78 and 152 of U.S. Pat. No. 8,734,809), AAV CKd-B7 (SEQ ID NO: 79 and 153 of U88734809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809), AAV CKd-H2 (SEQ ID NO: 82 and 156 of U88734809), AAV CKd-H3 (SEQ ID NO: 83 and 157 of U88734809), AAV CKd-H4 (SEQ ID NO: 84 and 158 of U88734809), AAV CKd-H5 (SEQ ID NO: 85 and 159 of U88734809), AAV CKd-H6 (SEQ ID NO: 77 and 151 of U.S. Pat. No. 8,734,809), AAV CHt-1 (SEQ ID NO: 86 and 160 of U88734809), AAV CLv1-1 (SEQ ID NO: 171 of U88734809), AAV CLv1.2 (SEQ ID NO: 172 of U88734809), AAV CLv1-3 (SEQ ID NO: 173 of U88734809), AAV CLv1-4 (SEQ ID NO: 174 of U.S. Pat. No. 8,734,809), AAV Clv1-7 (SEQ ID NO: 175 of U88734809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U88734809), AAV.VR-355 (SEQ ID NO: 181 of U88734809), AAV.hu.48R3 (SEQ ID NO: 183 of U.S. Pat. No. 8,734,809), or variants or derivatives thereof.

In some embodiments, the AAV serotype may be, or have, a sequence as described in International Publication No. WO2016065001, the contents of which are herein incorporated by reference in their entirety, such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ ID NO: 12 and 62 of WO2016065001), AAV CKd-N4 (SEQ ID NO: 13 and 63 of WO2016065001), AAV CKd-N9 (SEQ ID NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV CLv-K6 (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CHt-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001), AAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ ID NO: 49 and 99 of WO2016065001), AAV5 (SEQ ID NO: 50 and 100 of WO2016065001), or variants or derivatives thereof.

In some embodiments, the AAV particle may have, or may be a serotype selected from any of those found in Table 1.

In some embodiments, the AAV capsid may comprise a sequence, fragment or variant thereof, of any of the sequences in Table 1.

In some embodiments, the AAV capsid may be encoded by a sequence, fragment or variant as described in Table 1.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gin) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; 0 (Pyl) for Pyrrolysine; U (Sec) for Selenocysteine; X (Xaa) for any amino acid; and Z (Glx) for Glutamine or Glutamic acid.

TABLE 1
AAV Serotypes

| | SEQ | |
|---|---|---|
| Serotype | ID NO: | Reference Information |
| VOY101 | 1 | — |
| VOY101 | 2 | — |
| VOY201 | 3 | — |
| VOY201 | 13140 | — |
| PHP.N/PHP.B-DGT | 4 | WO2017100671 SEQ ID NO: 46 |
| AAVPHP.B or G2B-26 | 5 | WO2015038958 SEQ ID NO: 8 and 13 |
| AAVPHP.B | 6 | WO2015038958 SEQ ID NO: 9 |
| AAVG2B-13 | 7 | WO2015038958 SEQ ID NO: 12 |
| AAVTH1.1-32 | 8 | WO2015038958 SEQ ID NO: 14 |
| AAVTH1.1-35 | 9 | WO2015038958 SEQ ID NO: 15 |
| PHP.S/G2A12 | 10 | WO2017100671 SEQ ID NO: 47 |
| AAV9/hu.14 K449R | 11 | WO2017100671 SEQ ID NO: 45 |
| AAV1 | 12 | US20150159173 SEQ ID NO: 11, US20150315612 SEQ ID NO: 202 |
| AAV1 | 13 | US20160017295 SEQ ID NO: 1, US20030138772 SEQ ID NO: 64, |
| | | US20150159173 SEQ ID NO: 27, US20150315612 SEQ ID NO: 219, |
| | | U.S. Pat. No. 7,198,951 SEQ ID NO: 5 |
| AAV1 | 14 | US20030138772 SEQ ID NO: 6 |
| AAV1.3 | 15 | US20030138772 SEQ ID NO: 14 |
| AAV10 | 16 | US20030138772 SEQ ID NO: 117 |
| AAV10 | 17 | WO2015121501 SEQ ID NO: 9 |
| AAV10 | 18 | WO2015121501 SEQ ID NO: 8 |
| AAV11 | 19 | US20030138772 SEQ ID NO: 118 |
| AAV12 | 20 | US20030138772 SEQ ID NO: 119 |
| AAV2 | 21 | US20150159173 SEQ ID NO: 7, US20150315612 SEQ ID NO: 211 |
| AAV2 | 22 | US20030138772 SEQ ID NO: 70, US20150159173 SEQ ID NO: 23, |
| | | US20150315612 SEQ ID NO: 221, US20160017295 SEQ ID NO: 2, |
| | | U.S. Pat. No. 6,156,303 SEQ ID NO: 4, U.S. Pat. No. 7,198,951 SEQ ID NO: 4, |
| | | WO2015121501 SEQ ID NO: 1 |
| AAV2 | 23 | U.S. Pat. No. 6,156,303 SEQ ID NO: 8 |
| AAV2 | 24 | US20030138772 SEQ ID NO: 7 |
| AAV2 | 25 | U.S. Pat. No. 6,156,303 SEQ ID NO: 3 |
| AAV2.5T | 26 | U.S. Pat. No. 9,233,131 SEQ ID NO: 42 |
| AAV223.10 | 27 | US20030138772 SEQ ID NO: 75 |
| AAV223.2 | 28 | US20030138772 SEQ ID NO: 49 |
| AAV223.2 | 29 | US20030138772 SEQ ID NO: 76 |
| AAV223.4 | 30 | US20030138772 SEQ ID NO: 50 |
| AAV223.4 | 31 | US20030138772 SEQ ID NO: 73 |
| AAV223.5 | 32 | US20030138772 SEQ ID NO: 51 |
| AAV223.5 | 33 | US20030138772 SEQ ID NO: 74 |
| AAV223.6 | 34 | US20030138772 SEQ ID NO: 52 |
| AAV223.6 | 35 | US20030138772 SEQ ID NO: 78 |
| AAV223.7 | 36 | US20030138772 SEQ ID NO: 53 |
| AAV223.7 | 37 | US20030138772 SEQ ID NO: 77 |
| AAV29.3 | 38 | US20030138772 SEQ ID NO: 82 |
| AAV29.4 | 39 | US20030138772 SEQ ID NO: 12 |
| AAV29.5 | 40 | US20030138772 SEQ ID NO: 83 |
| AAV29.5 (AAVbb.2) | 41 | US20030138772 SEQ ID NO: 13 |
| AAV3 | 42 | US20150159173 SEQ ID NO: 12 |
| AAV3 | 43 | US20030138772 SEQ ID NO: 71, US20150159173 SEQ ID NO: 28, |
| | | US20160017295 SEQ ID NO: 3, U.S. Pat. No. 7,198,951 SEQ ID NO: 6 |
| AAV3 | 44 | US20030138772 SEQ ID NO: 8 |
| AAV3.3b | 45 | US20030138772 SEQ ID NO: 72 |
| AAV3-3 | 46 | US20150315612 SEQ ID NO: 200 |
| AAV3-3 | 47 | US20150315612 SEQ ID NO: 217 |
| AAV3a | 48 | U.S. Pat. No. 6,156,303 SEQ ID NO: 5 |
| AAV3a | 49 | U.S. Pat. No. 6,156,303 SEQ ID NO: 9 |
| AAV3b | 50 | U.S. Pat. No. 6,156,303 SEQ ID NO: 6 |
| AAV3b | 51 | U.S. Pat. No. 6,156,303 SEQ ID NO: 10 |
| AAV3b | 52 | U.S. Pat. No. 6,156,303 SEQ ID NO: 1 |
| AAV4 | 53 | US20140348794 SEQ ID NO: 17 |
| AAV4 | 54 | US20140348794 SEQ ID NO: 5 |
| AAV4 | 55 | US20140348794 SEQ ID NO: 3 |
| AAV4 | 56 | US20140348794 SEQ ID NO: 14 |
| AAV4 | 57 | US20140348794 SEQ ID NO: 15 |
| AAV4 | 58 | US20140348794 SEQ ID NO: 19 |
| AAV4 | 59 | US20140348794 SEQ ID NO: 12 |
| AAV4 | 60 | US20140348794 SEQ ID NO: 13 |
| AAV4 | 61 | US20140348794 SEQ ID NO: 7 |
| AAV4 | 62 | US20140348794 SEQ ID NO: 8 |
| AAV4 | 63 | US20140348794 SEQ ID NO: 9 |
| AAV4 | 64 | US20140348794 SEQ ID NO: 2 |
| AAV4 | 65 | US20140348794 SEQ ID NO: 10 |
| AAV4 | 66 | US20140348794 SEQ ID NO: 11 |
| AAV4 | 67 | US20140348794 SEQ ID NO: 18 |
| AAV4 | 68 | US20030138772 SEQ ID NO: 63, US20160017295 SEQ ID NO: 4, |
| | | US20140348794 SEQ ID NO: 4 |
| AAV4 | 69 | US20140348794 SEQ ID NO: 16 |
| AAV4 | 70 | US20140348794 SEQ ID NO: 20 |
| AAV4 | 71 | US20140348794 SEQ ID NO: 6 |
| AAV4 | 72 | US20140348794 SEQ ID NO: 1 |
| AAV42.2 | 73 | US20030138772 SEQ ID NO: 9 |
| AAV42.2 | 74 | US20030138772 SEQ ID NO: 102 |
| AAV42.3b | 75 | US20030138772 SEQ ID NO: 36 |
| AAV42.3B | 76 | US20030138772 SEQ ID NO: 107 |
| AAV42.4 | 77 | US20030138772 SEQ ID NO: 33 |
| AAV42.4 | 78 | US20030138772 SEQ ID NO: 88 |
| AAV42.8 | 79 | US20030138772 SEQ ID NO: 27 |
| AAV42.8 | 80 | US20030138772 SEQ ID NO: 85 |
| AAV43.1 | 81 | US20030138772 SEQ ID NO: 39 |
| AAV43.1 | 82 | US20030138772 SEQ ID NO: 92 |
| AAV43.12 | 83 | US20030138772 SEQ ID NO: 41 |
| AAV43.12 | 84 | US20030138772 SEQ ID NO: 93 |
| AAV43.20 | 85 | US20030138772 SEQ ID NO: 42 |
| AAV43.20 | 86 | US20030138772 SEQ ID NO: 99 |
| AAV43.21 | 87 | US20030138772 SEQ ID NO: 43 |
| AAV43.21 | 88 | US20030138772 SEQ ID NO: 96 |
| AAV43.23 | 89 | US20030138772 SEQ ID NO: 44 |
| AAV43.23 | 90 | US20030138772 SEQ ID NO: 98 |
| AAV43.25 | 91 | US20030138772 SEQ ID NO: 45 |
| AAV43.25 | 92 | US20030138772 SEQ ID NO: 97 |
| AAV43.5 | 93 | US20030138772 SEQ ID NO: 40 |
| AAV43.5 | 94 | US20030138772 SEQ ID NO: 94 |
| AAV4-4 | 95 | US20150315612 SEQ ID NO: 201 |
| AAV4-4 | 96 | US20150315612 SEQ ID NO: 218 |
| AAV44.1 | 97 | US20030138772 SEQ ID NO: 46 |
| AAV44.1 | 98 | US20030138772 SEQ ID NO: 79 |
| AAV44.5 | 99 | US20030138772 SEQ ID NO: 47 |
| AAV44.5 | 100 | US20030138772 SEQ ID NO: 80 |
| AAV4407 | 101 | US20150315612 SEQ ID NO: 90 |
| AAV5 | 102 | U.S. Pat. No. 7,427,396 SEQ ID NO: 1 |
| AAV5 | 103 | US20030138772 SEQ ID NO: 114 |
| AAV5 | 104 | US20160017295 SEQ ID NO: 5, U.S. Pat. No. 7,427,396 SEQ ID NO: 2, |
| | | US20150315612 SEQ ID NO: 216 |
| AAV5 | 105 | US20150315612 SEQ ID NO: 199 |
| AAV6 | 106 | US20150159173 SEQ ID NO: 13 |
| AAV6 | 107 | US20030138772 SEQ ID NO: 65, US20150159173 SEQ ID NO: 29, |
| | | US20160017295 SEQ ID NO: 6, U.S. Pat. No. 6,156,303 SEQ ID NO: 7 |
| AAV6 | 108 | U.S. Pat. No. 6,156,303 SEQ ID NO: 11 |
| AAV6 | 109 | U.S. Pat. No. 6,156,303 SEQ ID NO: 2 |
| AAV6 | 110 | US20150315612 SEQ ID NO: 203 |
| AAV6 | 111 | US20150315612 SEQ ID NO: 220 |
| AAV6.1 | 112 | US20150159173 |
| AAV6.12 | 113 | US20150159173 |
| AAV6.2 | 114 | US20150159173 |
| AAV7 | 115 | US20150159173 SEQ ID NO: 14 |
| AAV7 | 116 | US20150315612 SEQ ID NO: 183 |
| AAV7 | 117 | US20030138772 SEQ ID NO: 2, US20150159173 SEQ ID NO: 30, |
| | | US20150315612 SEQ ID NO: 181, US20160017295 SEQ ID NO: 7 |
| AAV7 | 118 | US20030138772 SEQ ID NO: 3 |
| AAV7 | 119 | US20030138772 SEQ ID NO: 1, US20150315612 SEQ ID NO: 180 |
| AAV7 | 120 | US20150315612 SEQ ID NO: 213 |
| AAV7 | 121 | US20150315612 SEQ ID NO: 222 |
| AAV8 | 122 | US20150159173 SEQ ID NO: 15 |
| AAV8 | 123 | US20150376240 SEQ ID NO: 7 |
| AAV8 | 124 | US20030138772 SEQ ID NO: 4, US20150315612 SEQ ID NO: 182 |
| AAV8 | 125 | US20030138772 SEQ ID NO: 95, US20140359799 SEQ ID NO: 1, |
| | | US20150159173 SEQ ID NO: 31, US20160017295 SEQ ID NO: 8, |
| | | U.S. Pat. No. 7,198,951 SEQ ID NO: 7, US20150315612 SEQ ID NO: 223 |
| AAV8 | 126 | US20150376240 SEQ ID NO: 8 |
| AAV8 | 127 | US20150315612 SEQ ID NO: 214 |
| AAV-8b | 128 | US20150376240 SEQ ID NO: 5 |
| AAV-8b | 129 | US20150376240 SEQ ID NO: 3 |
| AAV-8h | 130 | US20150376240 SEQ ID NO: 6 |
| AAV-8h | 131 | US20150376240 SEQ ID NO: 4 |
| AAV9 | 132 | US20030138772 SEQ ID NO: 5 |
| AAV9 | 133 | U.S. Pat. No. 7,198,951 SEQ ID NO: 1 |
| AAV9 | 134 | US20160017295 SEQ ID NO: 9 |
| AAV9 | 135 | US20030138772 SEQ ID NO: 100, U.S. Pat. No. 7,198,951 SEQ ID NO: 2 |
| AAV9 | 136 | U.S. Pat. No. 7,198,951 SEQ ID NO: 3 |
| AAV9 (AAVhu.14) | 137 | U.S. Pat. No. 7,906,111 SEQ ID NO: 3; WO2015038958 SEQ ID NO: 11 |
| AAV9 (AAVhu.14) | 138 | U.S. Pat. No. 7,906,111 SEQ ID NO: 123; WO2015038958 SEQ ID NO: 2 |
| AAVA3.1 | 139 | US20030138772 SEQ ID NO: 120 |
| AAVA3.3 | 140 | US20030138772 SEQ ID NO: 57 |
| AAVA3.3 | 141 | US20030138772 SEQ ID NO: 66 |
| AAVA3.4 | 142 | US20030138772 SEQ ID NO: 54 |
| AAVA3.4 | 143 | US20030138772 SEQ ID NO: 68 |
| AAVA3.5 | 144 | US20030138772 SEQ ID NO: 55 |
| AAVA3.5 | 145 | US20030138772 SEQ ID NO: 69 |
| AAVA3.7 | 146 | US20030138772 SEQ ID NO: 56 |
| AAVA3.7 | 147 | US20030138772 SEQ ID NO: 67 |
| AAV29.3 (AAVbb.1) | 148 | US20030138772 SEQ ID NO: 11 |
| AAVC2 | 149 | US20030138772 SEQ ID NO: 61 |
| AAVCh.5 | 150 | US20150159173 SEQ ID NO: 46, US20150315612 SEQ ID NO: 234 |
| AAVcy.2 (AAV13.3) | 151 | US20030138772 SEQ ID NO: 15 |
| AAV24.1 | 152 | US20030138772 SEQ ID NO: 101 |
| AAVcy.3 (AAV24.1) | 153 | US20030138772 SEQ ID NO: 16 |
| AAV27.3 | 154 | US20030138772 SEQ ID NO: 104 |
| AAVcy.4 (AAV27.3) | 155 | US20030138772 SEQ ID NO: 17 |
| AAVcy.5 | 156 | US20150315612 SEQ ID NO: 227 |
| AAV7.2 | 157 | US20030138772 SEQ ID NO: 103 |
| AAVcy.5 (AAV7.2) | 158 | US20030138772 SEQ ID NO: 18 |
| AAV16.3 | 159 | US20030138772 SEQ ID NO: 105 |
| AAVcy.6 (AAV16.3) | 160 | US20030138772 SEQ ID NO: 10 |
| AAVcy.5 | 161 | US20150159173 SEQ ID NO: 8 |
| AAVcy.5 | 162 | US20150159173 SEQ ID NO: 24 |
| AAVCy.5R1 | 163 | US20150159173 |
| AAVCy.5R2 | 164 | US20150159173 |
| AAVCy.5R3 | 165 | US20150159173 |
| AAVCy.5R4 | 166 | US20150159173 |
| AAVDJ | 167 | US20140359799 SEQ ID NO: 3, U.S. Pat. No. 7,588,772 SEQ ID NO: 2 |
| AAVDJ | 168 | US20140359799 SEQ ID NO: 2, U.S. Pat. No. 7,588,772 SEQ ID NO: 1 |
| AAVDJ-8 | 169 | U.S. Pat. No. 7,588,772; Grimm et al 2008 |
| AAVDJ-8 | 170 | U.S. Pat. No. 7,588,772; Grimm et al 2008 |
| AAVF5 | 171 | US20030138772 SEQ ID NO: 110 |
| AAVH2 | 172 | US20030138772 SEQ ID NO: 26 |
| AAVH6 | 173 | US20030138772 SEQ ID NO: 25 |
| AAVhE1.1 | 174 | U.S. Pat. No. 9,233,131 SEQ ID NO: 44 |
| AAVhEr1.14 | 175 | U.S. Pat. No. 9,233,131 SEQ ID NO: 46 |
| AAVhEr1.16 | 176 | U.S. Pat. No. 9,233,131 SEQ ID NO: 48 |
| AAVhEr1.18 | 177 | U.S. Pat. No. 9,233,131 SEQ ID NO: 49 |
| AAVhEr1.23 (AAVhEr2.29) | 178 | U.S. Pat. No. 9,233,131 SEQ ID NO: 53 |
| AAVhEr1.35 | 179 | U.S. Pat. No. 9,233,131 SEQ ID NO: 50 |
| AAVhEr1.36 | 180 | U.S. Pat. No. 9,233,131 SEQ ID NO: 52 |
| AAVhEr1.5 | 181 | U.S. Pat. No. 9,233,131 SEQ ID NO: 45 |
| AAVhEr1.7 | 182 | U.S. Pat. No. 9,233,131 SEQ ID NO: 51 |
| AAVhEr1.8 | 183 | U.S. Pat. No. 9,233,131 SEQ ID NO: 47 |
| AAVhEr2.16 | 184 | U.S. Pat. No. 9,233,131 SEQ ID NO: 55 |
| AAVhEr2.30 | 185 | U.S. Pat. No. 9,233,131 SEQ ID NO: 56 |
| AAVhEr2.31 | 186 | U.S. Pat. No. 9,233,131 SEQ ID NO: 58 |
| AAVhEr2.36 | 187 | U.S. Pat. No. 9,233,131 SEQ ID NO: 57 |
| AAVhEr2.4 | 188 | U.S. Pat. No. 9,233,131 SEQ ID NO: 54 |
| AAVhEr3.1 | 189 | U.S. Pat. No. 9,233,131 SEQ ID NO: 59 |
| AAVhu.1 | 190 | US20150315612 SEQ ID NO: 46 |
| AAVhu.1 | 191 | US20150315612 SEQ ID NO: 144 |
| AAVhu.10 (AAV16.8) | 192 | US20150315612 SEQ ID NO: 56 |
| AAVhu.10 (AAV16.8) | 193 | US20150315612 SEQ ID NO: 156 |
| AAVhu.11 (AAV16.12) | 194 | US20150315612 SEQ ID NO: 57 |
| AAVhu.11 (AAV16.12) | 195 | US20150315612 SEQ ID NO: 153 |
| AAVhu.12 | 196 | US20150315612 SEQ ID NO: 59 |
| AAVhu.12 | 197 | US20150315612 SEQ ID NO: 154 |
| AAVhu.13 | 198 | US20150159173 SEQ ID NO: 16, US20150315612 SEQ ID NO: 71 |
| AAVhu.13 | 199 | US20150159173 SEQ ID NO: 32, US20150315612 SEQ ID NO: 129 |
| AAVhu.136.1 | 200 | US20150315612 SEQ ID NO: 165 |
| AAVhu.140.1 | 201 | US20150315612 SEQ ID NO: 166 |
| AAVhu.140.2 | 202 | US20150315612 SEQ ID NO: 167 |
| AAVhu.145.6 | 203 | US20150315612 SEQ ID No: 178 |
| AAVhu.15 | 204 | US20150315612 SEQ ID NO: 147 |
| AAVhu.15 (AAV33.4) | 205 | US20150315612 SEQ ID NO: 50 |
| AAVhu.156.1 | 206 | US20150315612 SEQ ID No: 179 |
| AAVhu.16 | 207 | US20150315612 SEQ ID NO: 148 |
| AAVhu.16 (AAV33.8) | 208 | US20150315612 SEQ ID NO: 51 |
| AAVhu.17 | 209 | US20150315612 SEQ ID NO: 83 |
| AAVhu.17 (AAV33.12) | 210 | US20150315612 SEQ ID NO: 4 |
| AAVhu.172.1 | 211 | US20150315612 SEQ ID NO: 171 |
| AAVhu.172.2 | 212 | US20150315612 SEQ ID NO: 172 |
| AAVhu.173.4 | 213 | US20150315612 SEQ ID NO: 173 |
| AAVhu.173.8 | 214 | US20150315612 SEQ ID NO: 175 |
| AAVhu.18 | 215 | US20150315612 SEQ ID NO: 52 |
| AAVhu.18 | 216 | US20150315612 SEQ ID NO: 149 |
| AAVhu.19 | 217 | US20150315612 SEQ ID NO: 62 |
| AAVhu.19 | 218 | US20150315612 SEQ ID NO: 133 |
| AAVhu.2 | 219 | US20150315612 SEQ ID NO: 48 |
| AAVhu.2 | 220 | US20150315612 SEQ ID NO: 143 |
| AAVhu.20 | 221 | US20150315612 SEQ ID NO: 63 |
| AAVhu.20 | 222 | US20150315612 SEQ ID NO: 134 |
| AAVhu.21 | 223 | US20150315612 SEQ ID NO: 65 |
| AAVhu.21 | 224 | US20150315612 SEQ ID NO: 135 |
| AAVhu.22 | 225 | US20150315612 SEQ ID NO: 67 |
| AAVhu.22 | 226 | US20150315612 SEQ ID NO: 138 |
| AAVhu.23 | 227 | US20150315612 SEQ ID NO: 60 |
| AAVhu.23.2 | 228 | US20150315612 SEQ ID NO: 137 |
| AAVhu.24 | 229 | US20150315612 SEQ ID NO: 66 |
| AAVhu.24 | 230 | US20150315612 SEQ ID NO: 136 |
| AAVhu.25 | 231 | US20150315612 SEQ ID NO: 49 |
| AAVhu.25 | 232 | US20150315612 SEQ ID NO: 146 |
| AAVhu.26 | 233 | US20150159173 SEQ ID NO: 17, US20150315612 SEQ ID NO: 61 |
| AAVhu.26 | 234 | US20150159173 SEQ ID NO: 33, US20150315612 SEQ ID NO: 139 |
| AAVhu.27 | 235 | US20150315612 SEQ ID NO: 64 |
| AAVhu.27 | 236 | US20150315612 SEQ ID NO: 140 |
| AAVhu.28 | 237 | US20150315612 SEQ ID NO: 68 |
| AAVhu.28 | 238 | US20150315612 SEQ ID NO: 130 |
| AAVhu.29 | 239 | US20150315612 SEQ ID NO: 69 |
| AAVhu.29 | 240 | US20150159173 SEQ ID NO: 42, US20150315612 SEQ ID NO: 132 |
| AAVhu.29 | 241 | US20150315612 SEQ ID NO: 225 |
| AAVhu.29R | 242 | US20150159173 |
| AAVhu.3 | 243 | US20150315612 SEQ ID NO: 44 |
| AAVhu.3 | 244 | US20150315612 SEQ ID NO: 145 |
| AAVhu.30 | 245 | US20150315612 SEQ ID NO: 70 |
| AAVhu.30 | 246 | US20150315612 SEQ ID NO: 131 |
| AAVhu.31 | 247 | US20150315612 SEQ ID NO: 1 |
| AAVhu.31 | 248 | US20150315612 SEQ ID NO: 121 |
| AAVhu.32 | 249 | US20150315612 SEQ ID NO: 2 |
| AAVhu.32 | 250 | US20150315612 SEQ ID NO: 122 |
| AAVhu.33 | 251 | US20150315612 SEQ ID NO: 75 |
| AAVhu.33 | 252 | US20150315612 SEQ ID NO: 124 |
| AAVhu.34 | 253 | US20150315612 SEQ ID NO: 72 |
| AAVhu.34 | 254 | US20150315612 SEQ ID NO: 125 |
| AAVhu.35 | 255 | US20150315612 SEQ ID NO: 73 |
| AAVhu.35 | 256 | US20150315612 SEQ ID NO: 164 |
| AAVhu.36 | 257 | US20150315612 SEQ ID NO: 74 |
| AAVhu.36 | 258 | US20150315612 SEQ ID NO: 126 |
| AAVhu.37 | 259 | US20150159173 SEQ ID NO: 34, US20150315612 SEQ ID NO: 88 |
| AAVhu.37 (AAV106.1) | 260 | US20150315612 SEQ ID NO: 10, US20150159173 SEQ ID NO: 18 |
| AAVhu.38 | 261 | US20150315612 SEQ ID NO: 161 |
| AAVhu.39 | 262 | US20150315612 SEQ ID NO: 102 |
| AAVhu.39 (AAVLG-9) | 263 | US20150315612 SEQ ID NO: 24 |
| AAVhu.4 | 264 | US20150315612 SEQ ID NO: 47 |
| AAVhu.4 | 265 | US20150315612 SEQ ID NO: 141 |
| AAVhu.40 | 266 | US20150315612 SEQ ID NO: 87 |
| AAVhu.40 (AAV114.3) | 267 | US20150315612 SEQ ID No. 11 |
| AAVhu.41 | 268 | US20150315612 SEQ ID NO: 91 |
| AAVhu.41 (AAV127.2) | 269 | US20150315612 SEQ ID NO: 6 |
| AAVhu.42 | 270 | US20150315612 SEQ ID NO: 85 |
| AAVhu.42 (AAV127.5) | 271 | US20150315612 SEQ ID NO: 8 |
| AAVhu.43 | 272 | US20150315612 SEQ ID NO: 160 |
| AAVhu.43 | 273 | US20150315612 SEQ ID NO: 236 |
| AAVhu.43 (AAV128.1) | 274 | US20150315612 SEQ ID NO: 80 |
| AAVhu.44 | 275 | US20150159173 SEQ ID NO: 45, US20150315612 SEQ ID NO: 158 |
| AAVhu.44 (AAV128.3) | 276 | US20150315612 SEQ ID NO: 81 |
| AAVhu.44R1 | 277 | US20150159173 |
| AAVhu.44R2 | 278 | US20150159173 |
| AAVhu.44R3 | 279 | US20150159173 |
| AAVhu.45 | 280 | US20150315612 SEQ ID NO: 76 |
| AAVhu.45 | 281 | US20150315612 SEQ ID NO: 127 |
| AAVhu.46 | 282 | US20150315612 SEQ ID NO: 82 |
| AAVhu.46 | 283 | US20150315612 SEQ ID NO: 159 |
| AAVhu.46 | 284 | US20150315612 SEQ ID NO: 224 |
| AAVhu.47 | 285 | US20150315612 SEQ ID NO: 77 |
| AAVhu.47 | 286 | US20150315612 SEQ ID NO: 128 |
| AAVhu.48 | 287 | US20150159173 SEQ ID NO: 38 |
| AAVhu.48 | 288 | US20150315612 SEQ ID NO: 157 |
| AAVhu.48 (AAV130.4) | 289 | US20150315612 SEQ ID NO: 78 |
| AAVhu.48R1 | 290 | US20150159173 |
| AAVhu.48R2 | 291 | US20150159173 |
| AAVhu.48R3 | 292 | US20150159173 |
| AAVhu.49 | 293 | US20150315612 SEQ ID NO: 209 |
| AAVhu.49 | 294 | US20150315612 SEQ ID NO: 189 |
| AAVhu.5 | 295 | US20150315612 SEQ ID NO: 45 |
| AAVhu.5 | 296 | US20150315612 SEQ ID NO: 142 |
| AAVhu.51 | 297 | US20150315612 SEQ ID NO: 208 |
| AAVhu.51 | 298 | US20150315612 SEQ ID NO: 190 |
| AAVhu.52 | 299 | US20150315612 SEQ ID NO: 210 |
| AAVhu.52 | 300 | US20150315612 SEQ ID NO: 191 |
| AAVhu.53 | 301 | US20150159173 SEQ ID NO: 19 |
| AAVhu.53 | 302 | US20150159173 SEQ ID NO: 35 |
| AAVhu.53 (AAV145.1) | 303 | US20150315612 SEQ ID NO: 176 |
| AAVhu.54 | 304 | US20150315612 SEQ ID NO: 188 |
| AAVhu.54 (AAV145.5) | 305 | US20150315612 SEQ ID No: 177 |
| AAVhu.55 | 306 | US20150315612 SEQ ID NO: 187 |
| AAVhu.56 | 307 | US20150315612 SEQ ID NO: 205 |
| AAVhu.56 (AAV145.6) | 308 | US20150315612 SEQ ID NO: 168 |
| AAVhu.56 (AAV145.6) | 309 | US20150315612 SEQ ID NO: 192 |
| AAVhu.57 | 310 | US20150315612 SEQ ID NO: 206 |
| AAVhu.57 | 311 | US20150315612 SEQ ID NO: 169 |
| AAVhu.57 | 312 | US20150315612 SEQ ID NO: 193 |
| AAVhu.58 | 313 | US20150315612 SEQ ID NO: 207 |
| AAVhu.58 | 314 | US20150315612 SEQ ID NO: 194 |
| AAVhu.6 (AAV3.1) | 315 | US20150315612 SEQ ID NO: 5 |
| AAVhu.6 (AAV3.1) | 316 | US20150315612 SEQ ID NO: 84 |
| AAVhu.60 | 317 | US20150315612 SEQ ID NO: 184 |
| AAVhu.60 (AAV161.10) | 318 | US20150315612 SEQ ID NO: 170 |
| AAVhu.61 | 319 | US20150315612 SEQ ID NO: 185 |
| AAVhu.61 (AAV161.6) | 320 | US20150315612 SEQ ID NO: 174 |
| AAVhu.63 | 321 | US20150315612 SEQ ID NO: 204 |
| AAVhu.63 | 322 | US20150315612 SEQ ID NO: 195 |
| AAVhu.64 | 323 | US20150315612 SEQ ID NO: 212 |
| AAVhu.64 | 324 | US20150315612 SEQ ID NO: 196 |
| AAVhu.66 | 325 | US20150315612 SEQ ID NO: 197 |
| AAVhu.67 | 326 | US20150315612 SEQ ID NO: 215 |
| AAVhu.67 | 327 | US20150315612 SEQ ID NO: 198 |
| AAVhu.7 | 328 | US20150315612 SEQ ID NO: 226 |
| AAVhu.7 | 329 | US20150315612 SEQ ID NO: 150 |
| AAVhu.7 (AAV7.3) | 330 | US20150315612 SEQ ID NO: 55 |
| AAVhu.71 | 331 | US20150315612 SEQ ID NO: 79 |
| AAVhu.8 | 332 | US20150315612 SEQ ID NO: 53 |
| AAVhu.8 | 333 | US20150315612 SEQ ID NO: 12 |
| AAVhu.8 | 334 | US20150315612 SEQ ID NO: 151 |
| AAVhu.9 (AAV3.1) | 335 | US20150315612 SEQ ID NO: 58 |
| AAVhu.9 (AAV3.1) | 336 | US20150315612 SEQ ID NO: 155 |
| AAV-LK01 | 337 | US20150376607 SEQ ID NO: 2 |
| AAV-LK01 | 338 | US20150376607 SEQ ID NO: 29 |
| AAV-LK02 | 339 | US20150376607 SEQ ID NO: 3 |
| AAV-LK02 | 340 | US20150376607 SEQ ID NO: 30 |
| AAV-LK03 | 341 | US20150376607 SEQ ID NO: 4 |
| AAV-LK03 | 342 | WO2015121501 SEQ ID NO: 12, US20150376607 SEQ ID NO: 31 |
| AAV-LK04 | 343 | US20150376607 SEQ ID NO: 5 |
| AAV-LK04 | 344 | US20150376607 SEQ ID NO: 32 |
| AAV-LK05 | 345 | US20150376607 SEQ ID NO: 6 |
| AAV-LK05 | 346 | US20150376607 SEQ ID NO: 33 |
| AAV-LK06 | 347 | US20150376607 SEQ ID NO: 7 |
| AAV-LK06 | 348 | US20150376607 SEQ ID NO: 34 |
| AAV-LK07 | 349 | US20150376607 SEQ ID NO: 8 |
| AAV-LK07 | 350 | US20150376607 SEQ ID NO: 35 |
| AAV-LK08 | 351 | US20150376607 SEQ ID NO: 9 |
| AAV-LK08 | 352 | US20150376607 SEQ ID NO: 36 |
| AAV-LK09 | 353 | US20150376607 SEQ ID NO: 10 |
| AAV-LK09 | 354 | US20150376607 SEQ ID NO: 37 |
| AAV-LK10 | 355 | US20150376607 SEQ ID NO: 11 |
| AAV-LK10 | 356 | US20150376607 SEQ ID NO: 38 |
| AAV-LK11 | 357 | US20150376607 SEQ ID NO: 12 |
| AAV-LK11 | 358 | US20150376607 SEQ ID NO: 39 |
| AAV-LK12 | 359 | US20150376607 SEQ ID NO: 13 |
| AAV-LK12 | 360 | US20150376607 SEQ ID NO: 40 |
| AAV-LK13 | 361 | US20150376607 SEQ ID NO: 14 |
| AAV-LK13 | 362 | US20150376607 SEQ ID NO: 41 |
| AAV-LK14 | 363 | US20150376607 SEQ ID NO: 15 |
| AAV-LK14 | 364 | US20150376607 SEQ ID NO: 42 |
| AAV-LK15 | 365 | US20150376607 SEQ ID NO: 16 |
| AAV-LK15 | 366 | US20150376607 SEQ ID NO: 43 |
| AAV-LK16 | 367 | US20150376607 SEQ ID NO: 17 |
| AAV-LK16 | 368 | US20150376607 SEQ ID NO: 44 |
| AAV-LK17 | 369 | US20150376607 SEQ ID NO: 18 |
| AAV-LK17 | 370 | US20150376607 SEQ ID NO: 45 |
| AAV-LK18 | 371 | US20150376607 SEQ ID NO: 19 |
| AAV-LK18 | 372 | US20150376607 SEQ ID NO: 46 |
| AAV-LK19 | 373 | US20150376607 SEQ ID NO: 20 |
| AAV-LK19 | 374 | US20150376607 SEQ ID NO: 47 |
| AAV-PAEC | 375 | US20150376607 SEQ ID NO: 1 |
| AAV-PAEC | 376 | US20150376607 SEQ ID NO: 48 |
| AAV-PAEC11 | 377 | US20150376607 SEQ ID NO: 26 |
| AAV-PAEC11 | 378 | US20150376607 SEQ ID NO: 54 |
| AAV-PAEC12 | 379 | US20150376607 SEQ ID NO: 27 |
| AAV-PAEC12 | 380 | US20150376607 SEQ ID NO: 51 |
| AAV-PAEC13 | 381 | US20150376607 SEQ ID NO: 28 |
| AAV-PAEC13 | 382 | US20150376607 SEQ ID NO: 49 |
| AAV-PAEC2 | 383 | US20150376607 SEQ ID NO: 21 |
| AAV-PAEC2 | 384 | US20150376607 SEQ ID NO: 56 |
| AAV-PAEC4 | 385 | US20150376607 SEQ ID NO: 22 |
| AAV-PAEC4 | 386 | US20150376607 SEQ ID NO: 55 |
| AAV-PAEC6 | 387 | US20150376607 SEQ ID NO: 23 |
| AAV-PAEC6 | 388 | US20150376607 SEQ ID NO: 52 |
| AAV-PAEC7 | 389 | US20150376607 SEQ ID NO: 24 |
| AAV-PAEC7 | 390 | US20150376607 SEQ ID NO: 53 |
| AAV-PAEC8 | 391 | US20150376607 SEQ ID NO: 25 |
| AAV-PAEC8 | 392 | US20150376607 SEQ ID NO: 50 |
| AAVpi.1 | 393 | US20150315612 SEQ ID NO: 28 |
| AAVpi.1 | 394 | US20150315612 SEQ ID NO: 93 |
| AAVpi.2 | 395 | US20150315612 SEQ ID NO: 30 |
| AAVpi.2 | 396 | US20150315612 SEQ ID NO: 95 |
| AAVpi.3 | 397 | US20150315612 SEQ ID NO: 29 |
| AAVpi.3 | 398 | US20150315612 SEQ ID NO: 94 |
| AAVrh.10 | 399 | US20150159173 SEQ ID NO: 9 |
| AAVrh.10 | 400 | US20150159173 SEQ ID NO: 25 |
| AAV44.2 | 401 | US20030138772 SEQ ID NO: 59 |
| AAVrh.10 (AAV44.2) | 402 | US20030138772 SEQ ID NO: 81 |
| AAV42.1B | 403 | US20030138772 SEQ ID NO: 90 |
| AAVrh.12 (AAV42.1b) | 404 | US20030138772 SEQ ID NO: 30 |
| AAVrh.13 | 405 | US20150159173 SEQ ID NO: 10 |
| AAVrh.13 | 406 | US20150159173 SEQ ID NO: 26 |
| AAVrh.13 | 407 | US20150315612 SEQ ID NO: 228 |
| AAVrh.13R | 408 | US20150159173 |
| AAV42.3A | 409 | US20030138772 SEQ ID NO: 87 |
| AAVrh.14 (AAV42.3a) | 410 | US20030138772 SEQ ID NO: 32 |
| AAV42.5A | 411 | US20030138772 SEQ ID NO: 89 |
| AAVrh.17 (AAV42.5a) | 412 | US20030138772 SEQ ID NO: 34 |
| AAV42.5B | 413 | US20030138772 SEQ ID NO: 91 |
| AAVrh.18 (AAV42.5b) | 414 | US20030138772 SEQ ID NO: 29 |
| AAV42.6B | 415 | US20030138772 SEQ ID NO: 112 |
| AAVrh.19 (AAV42.6b) | 416 | US20030138772 SEQ ID NO: 38 |
| AAVrh.2 | 417 | US20150159173 SEQ ID NO: 39 |
| AAVrh.2 | 418 | US20150315612 SEQ ID NO: 231 |
| AAVrh.20 | 419 | US20150159173 SEQ ID NO: 1 |
| AAV42.10 | 420 | US20030138772 SEQ ID NO: 106 |
| AAVrh.21 (AAV42.10) | 421 | US20030138772 SEQ ID NO: 35 |
| AAV42.11 | 422 | US20030138772 SEQ ID NO: 108 |
| AAVrh.22 (AAV42.11) | 423 | US20030138772 SEQ ID NO: 37 |
| AAV42.12 | 424 | US20030138772 SEQ ID NO: 113 |
| AAVrh.23 (AAV42.12) | 425 | US20030138772 SEQ ID NO: 58 |
| AAV42.13 | 426 | US20030138772 SEQ ID NO: 86 |
| AAVrh.24 (AAV42.13) | 427 | US20030138772 SEQ ID NO: 31 |
| AAV42.15 | 428 | US20030138772 SEQ ID NO: 84 |
| AAVrh.25 (AAV42.15) | 429 | US20030138772 SEQ ID NO: 28 |
| AAVrh.2R | 430 | US20150159173 |
| AAVrh.31 (AAV223.1) | 431 | US20030138772 SEQ ID NO: 48 |
| AAVC1 | 432 | US20030138772 SEQ ID NO: 60 |
| AAVrh.32 (AAVC1) | 433 | US20030138772 SEQ ID NO: 19 |
| AAVrh.32/33 | 434 | US20150159173 SEQ ID NO: 2 |
| AAVrh.33 (AAVC3) | 435 | US20030138772 SEQ ID NO: 20 |
| AAVC5 | 436 | US20030138772 SEQ ID NO: 62 |
| AAVrh.34 (AAVC5) | 437 | US20030138772 SEQ ID NO: 21 |
| AAVF1 | 438 | US20030138772 SEQ ID NO: 109 |
| AAVrh.35 (AAVF1) | 439 | US20030138772 SEQ ID NO: 22 |
| AAVF3 | 440 | US20030138772 SEQ ID NO: 111 |
| AAVrh.36 (AAVF3) | 441 | US20030138772 SEQ ID NO: 23 |
| AAVrh.37 | 442 | US20030138772 SEQ ID NO: 24 |
| AAVrh.37 | 443 | US20150159173 SEQ ID NO: 40 |
| AAVrh.37 | 444 | US20150315612 SEQ ID NO: 229 |
| AAVrh.37R2 | 445 | US20150159173 |
| AAVrh.38 (AAVLG-4) | 446 | US20150315612 SEQ ID NO: 7 |
| AAVrh.38 (AAVLG-4) | 447 | US20150315612 SEQ ID NO: 86 |
| AAVrh.39 | 448 | US20150159173 SEQ ID NO: 20, US20150315612 SEQ ID NO: 13 |
| AAVrh.39 | 449 | US20150159173 SEQ ID NO: 3, US20150159173 SEQ ID NO: 36, |
| | | US20150315612 SEQ ID NO: 89 |
| AAVrh.40 | 450 | US20150315612 SEQ ID NO: 92 |
| AAVrh.40 (AAVLG-10) | 451 | US20150315612 SEQ ID No: 14 |
| AAVrh.43 (AAVN721-8) | 452 | US20150315612 SEQ ID NO: 43, US20150159173 SEQ ID NO: 21 |
| AAVrh.43 (AAVN721-8) | 453 | US20150315612 SEQ ID NO: 163, US20150159173 SEQ ID NO: 37 |
| AAVrh.44 | 454 | US20150315612 SEQ ID NO: 34 |
| AAVrh.44 | 455 | US20150315612 SEQ ID NO: 111 |
| AAVrh.45 | 456 | US20150315612 SEQ ID NO: 41 |
| AAVrh.45 | 457 | US20150315612 SEQ ID NO: 109 |
| AAVrh.46 | 458 | US20150159173 SEQ ID NO: 22, US20150315612 SEQ ID NO: 19 |
| AAVrh.46 | 459 | US20150159173 SEQ ID NO: 4, US20150315612 SEQ ID NO: 101 |
| AAVrh.47 | 460 | US20150315612 SEQ ID NO: 38 |
| AAVrh.47 | 461 | US20150315612 SEQ ID NO: 118 |
| AAVrh.48 | 462 | US20150159173 SEQ ID NO: 44, US20150315612 SEQ ID NO: 115 |
| AAVrh.48.1 | 463 | US20150159173 |
| AAVrh.48.1.2 | 464 | US20150159173 |
| AAVrh.48.2 | 465 | US20150159173 |
| AAVrh.48 (AAV1-7) | 466 | US20150315612 SEQ ID NO: 32 |
| AAVrh.49 (AAV1-8) | 467 | US20150315612 SEQ ID NO: 25 |
| AAVrh.49 (AAV1-8) | 468 | US20150315612 SEQ ID NO: 103 |
| AAVrh.50 (AAV2-4) | 469 | US20150315612 SEQ ID NO: 23 |
| AAVrh.50 (AAV2-4) | 470 | US20150315612 SEQ ID NO: 108 |
| AAVrh.51 (AAV2-5) | 471 | US20150315612 SEQ ID No: 22 |
| AAVrh.51 (AAV2-5) | 472 | US20150315612 SEQ ID NO: 104 |
| AAVrh.52 (AAV3-9) | 473 | US20150315612 SEQ ID NO: 18 |
| AAVrh.52 (AAV3-9) | 474 | US20150315612 SEQ ID NO: 96 |
| AAVrh.53 | 475 | US20150315612 SEQ ID NO: 97 |
| AAVrh.53 (AAV3-11) | 476 | US20150315612 SEQ ID NO: 17 |
| AAVrh.53 (AAV3-11) | 477 | US20150315612 SEQ ID NO: 186 |
| AAVrh.54 | 478 | US20150315612 SEQ ID NO: 40 |
| AAVrh.54 | 479 | US20150159173 SEQ ID NO: 49, US20150315612 SEQ ID NO: 116 |
| AAVrh.55 | 480 | US20150315612 SEQ ID NO: 37 |
| AAVrh.55 (AAV4-19) | 481 | US20150315612 SEQ ID NO: 117 |
| AAVrh.56 | 482 | US20150315612 SEQ ID NO: 54 |
| AAVrh.56 | 483 | US20150315612 SEQ ID NO: 152 |
| AAVrh.57 | 484 | US20150315612 SEQ ID NO: 26 |
| AAVrh.57 | 485 | US20150315612 SEQ ID NO: 105 |
| AAVrh.58 | 486 | US20150315612 SEQ ID NO: 27 |
| AAVrh.58 | 487 | US20150159173 SEQ ID NO: 48, US20150315612 SEQ ID NO: 106 |
| AAVrh.58 | 488 | US20150315612 SEQ ID NO: 232 |
| AAVrh.59 | 489 | US20150315612 SEQ ID NO: 42 |
| AAVrh.59 | 490 | US20150315612 SEQ ID NO: 110 |
| AAVrh.60 | 491 | US20150315612 SEQ ID NO: 31 |
| AAVrh.60 | 492 | US20150315612 SEQ ID NO: 120 |
| AAVrh.61 | 493 | US20150315612 SEQ ID NO: 107 |
| AAVrh.61 (AAV2-3) | 494 | US20150315612 SEQ ID NO: 21 |
| AAVrh.62 (AAV2-15) | 495 | US20150315612 SEQ ID No: 33 |
| AAVrh.62 (AAV2-15) | 496 | US20150315612 SEQ ID NO: 114 |
| AAVrh.64 | 497 | US20150315612 SEQ ID No: 15 |
| AAVrh.64 | 498 | US20150159173 SEQ ID NO: 43, US20150315612 SEQ ID NO: 99 |
| AAVrh.64 | 499 | US20150315612 SEQ ID NO: 233 |
| AAVRh.64R1 | 500 | US20150159173 |
| AAVRh.64R2 | 501 | US20150159173 |
| AAVrh.65 | 502 | US20150315612 SEQ ID NO: 35 |
| AAVrh.65 | 503 | US20150315612 SEQ ID NO: 112 |
| AAVrh.67 | 504 | US20150315612 SEQ ID NO: 36 |
| AAVrh.67 | 505 | US20150315612 SEQ ID NO: 230 |
| AAVrh.67 | 506 | US20150159173 SEQ ID NO: 47, US20150315612 SEQ ID NO: 113 |
| AAVrh.68 | 507 | US20150315612 SEQ ID NO: 16 |
| AAVrh.68 | 508 | US20150315612 SEQ ID NO: 100 |
| AAVrh.69 | 509 | US20150315612 SEQ ID NO: 39 |
| AAVrh.69 | 510 | US20150315612 SEQ ID NO: 119 |
| AAVrh.70 | 511 | US20150315612 SEQ ID NO: 20 |
| AAVrh.70 | 512 | US20150315612 SEQ ID NO: 98 |
| AAVrh.71 | 513 | US20150315612 SEQ ID NO: 162 |
| AAVrh.72 | 514 | US20150315612 SEQ ID NO: 9 |
| AAVrh.73 | 515 | US20150159173 SEQ ID NO: 5 |
| AAVrh.74 | 516 | US20150159173 SEQ ID NO: 6 |
| AAVrh.8 | 517 | US20150159173 SEQ ID NO: 41 |
| AAVrh.8 | 518 | US20150315612 SEQ ID NO: 235 |
| AAVrh.8R | 519 | US20150159173, WO2015168666 SEQ ID NO: 9 |
| AAVrh.8R A586R mutant | 520 | WO2015168666 SEQ ID NO: 10 |
| AAVrh.8R R533A mutant | 521 | WO2015168666 SEQ ID NO: 11 |
| BAAV (bovine AAV) | 522 | U.S. Pat. No. 9,193,769 SEQ ID NO: 8 |
| BAAV (bovine AAV) | 523 | U.S. Pat. No. 9,193,769 SEQ ID NO: 10 |
| BAAV (bovine AAA) | 524 | U.S. Pat. No. 9,193,769 SEQ ID NO: 4 |
| BAAV (bovine AAV) | 525 | U.S. Pat. No. 9,193,769 SEQ ID NO: 2 |
| BAAV (bovine AAV) | 526 | U.S. Pat. No. 9,193,769 SEQ ID NO: 6 |
| BAAV (bovine AAV) | 527 | U.S. Pat. No. 9,193,769 SEQ ID NO: 1 |
| BAAV (bovine AAV) | 528 | U.S. Pat. No. 9,193,769 SEQ ID NO: 5 |
| BAAV (bovine AAV) | 529 | U.S. Pat. No. 9,193,769 SEQ ID NO: 3 |
| BAAV (bovine AAV) | 530 | U.S. Pat. No. 9,193,769 SEQ ID NO: 11 |
| BAAV (bovine AAA) | 531 | U.S. Pat. No. 7,427,396 SEQ ID NO: 5 |
| BAAV (bovine AAV) | 532 | U.S. Pat. No. 7,427,396 SEQ ID NO: 6 |
| BAAV (bovine AAV) | 533 | U.S. Pat. No. 9,193,769 SEQ ID NO: 7 |
| BAAV (bovine AAV) | 534 | U.S. Pat. No. 9,193,769 SEQ ID NO: 9 |
| BNP61 AAV | 535 | US20150238550 SEQ ID NO: 1 |
| BNP61 AAV | 536 | US20150238550 SEQ ID NO: 2 |
| BNP62 AAV | 537 | US20150238550 SEQ ID NO: 3 |
| BNP63 AAV | 538 | US20150238550 SEQ ID NO: 4 |
| caprine AAV | 539 | U.S. Pat. No. 7,427,396 SEQ ID NO: 3 |
| caprine AAV | 540 | U.S. Pat. No. 7,427,396 SEQ ID NO: 4 |
| true type AAV (ttAAV) | 541 | WO2015121501 SEQ ID NO: 2 |
| AAAV (Avian AAV) | 542 | U.S. Pat. No. 9,238,800 SEQ ID NO: 12 |
| AAAV (Avian AAV) | 543 | U.S. Pat. No. 9,238,800 SEQ ID NO: 2 |
| AAAV (Avian AAV) | 544 | U.S. Pat. No. 9,238,800 SEQ ID NO: 6 |
| AAAV (Avian AAV) | 545 | U.S. Pat. No. 9,238,800 SEQ ID NO: 4 |
| AAAV (Avian AAV) | 546 | U.S. Pat. No. 9,238,800 SEQ ID NO: 8 |
| AAAV (Avian AAV) | 547 | U.S. Pat. No. 9,238,800 SEQ ID NO: 14 |
| AAAV (Avian AAV) | 548 | U.S. Pat. No. 9,238,800 SEQ ID NO: 10 |
| AAAV (Avian AAV) | 549 | U.S. Pat. No. 9,238,800 SEQ ID NO: 15 |
| AAAV (Avian AAV) | 550 | U.S. Pat. No. 9,238,800 SEQ ID NO: 5 |
| AAAV (Avian AAV) | 551 | U.S. Pat. No. 9,238,800 SEQ ID NO: 9 |
| AAAV (Avian AAV) | 552 | U.S. Pat. No. 9,238,800 SEQ ID NO: 3 |
| AAAV (Avian AAV) | 553 | U.S. Pat. No. 9,238,800 SEQ ID NO: 7 |
| AAAV (Avian AAV) | 554 | U.S. Pat. No. 9,238,800 SEQ ID NO: 11 |
| AAAV (Avian AAV) | 555 | U.S. Pat. No. 9,238,800 SEQ ID NO: 13 |
| AAAV (Avian AAV) | 556 | U.S. Pat. No. 9,238,800 SEQ ID NO: 1 |
| AAV Shuffle 100-1 | 557 | US20160017295 SEQ ID NO: 23 |
| AAV Shuffle 100-1 | 558 | US20160017295 SEQ ID NO: 11 |
| AAV Shuffle 100-2 | 559 | US20160017295 SEQ ID NO: 37 |
| AAV Shuffle 100-2 | 560 | US20160017295 SEQ ID NO: 29 |
| AAV Shuffle 100-3 | 561 | US20160017295 SEQ ID NO: 24 |
| AAV Shuffle 100-3 | 562 | US20160017295 SEQ ID NO: 12 |
| AAV Shuffle 100-7 | 563 | US20160017295 SEQ ID NO: 25 |
| AAV Shuffle 100-7 | 564 | US20160017295 SEQ ID NO: 13 |
| AAV Shuffle 10-2 | 565 | US20160017295 SEQ ID NO: 34 |
| AAV Shuffle 10-2 | 566 | US20160017295 SEQ ID NO: 26 |
| AAV Shuffle 10-6 | 567 | US20160017295 SEQ ID NO: 35 |
| AAV Shuffle 10-6 | 568 | US20160017295 SEQ ID NO: 27 |
| AAV Shuffle 10-8 | 569 | US20160017295 SEQ ID NO: 36 |
| AAV Shuffle 10-8 | 570 | US20160017295 SEQ ID NO: 28 |
| AAV SM 100-10 | 571 | US20160017295 SEQ ID NO: 41 |
| AAV SM 100-10 | 572 | US20160017295 SEQ ID NO: 33 |
| AAV SM 100-3 | 573 | US20160017295 SEQ ID NO: 40 |
| AAV SM 100-3 | 574 | US20160017295 SEQ ID NO: 32 |
| AAV SM 10-1 | 575 | US20160017295 SEQ ID NO: 38 |
| AAV SM 10-1 | 576 | US20160017295 SEQ ID NO: 30 |
| AAV SM 10-2 | 577 | US20160017295 SEQ ID NO: 10 |
| AAV SM 10-2 | 578 | US20160017295 SEQ ID NO: 22 |
| AAV SM 10-8 | 579 | US20160017295 SEQ ID NO: 39 |
| AAV SM 10-8 | 580 | US20160017295 SEQ ID NO: 31 |
| AAVF1/HSC1 | 581 | WO2016049230 SEQ ID NO: 20 |
| AAVF2/HSC2 | 582 | WO2016049230 SEQ ID NO: 21 |
| AAVF3/HSC3 | 583 | WO2016049230 SEQ ID NO: 22 |
| AAVF4/HSC4 | 584 | WO2016049230 SEQ ID NO: 23 |
| AAVF5/HSC5 | 585 | WO2016049230 SEQ ID NO: 25 |
| AAVF6/HSC6 | 586 | WO2016049230 SEQ ID NO: 24 |
| AAVF7/HSC7 | 587 | WO2016049230 SEQ ID NO: 27 |
| AAVF8/HSC8 | 588 | WO2016049230 SEQ ID NO: 28 |
| AAVF9/HSC9 | 589 | WO2016049230 SEQ ID NO: 29 |
| AAVF11/HSC11 | 590 | WO2016049230 SEQ ID NO: 26 |
| AAVF12/HSC12 | 591 | WO2016049230 SEQ ID NO: 30 |
| AAVF13/HSC13 | 592 | WO2016049230 SEQ ID NO: 31 |
| AAVF14/HSC14 | 593 | WO2016049230 SEQ ID NO: 32 |
| AAVF15/HSC15 | 594 | WO2016049230 SEQ ID NO: 33 |
| AAVF16/HSC16 | 595 | WO2016049230 SEQ ID NO: 34 |
| AAVF17/HSC17 | 596 | WO2016049230 SEQ ID NO: 35 |
| AAVF1/HSC1 | 597 | WO2016049230 SEQ ID NO: 2 |
| AAVF2/HSC2 | 598 | WO2016049230 SEQ ID NO: 3 |
| AAVF3/HSC3 | 599 | WO2016049230 SEQ ID NO: 5 |
| AAVF4/HSC4 | 600 | WO2016049230 SEQ ID NO: 6 |
| AAVF5/HSC5 | 601 | WO2016049230 SEQ ID NO: 11 |
| AAVF6/HSC6 | 602 | WO2016049230 SEQ ID NO: 7 |
| AAVF7/HSC7 | 603 | WO2016049230 SEQ ID NO: 8 |
| AAVF8/HSC8 | 604 | WO2016049230 SEQ ID NO: 9 |
| AAVF9/HSC9 | 605 | WO2016049230 SEQ ID NO: 10 |
| AAVF11/HSC11 | 606 | WO2016049230 SEQ ID NO: 4 |
| AAVF12/HSC12 | 607 | WO2016049230 SEQ ID NO: 12 |
| AAVF13/HSC13 | 608 | WO2016049230 SEQ ID NO: 14 |
| AAVF14/HSC14 | 609 | WO2016049230 SEQ ID NO: 15 |
| AAVF15/HSC15 | 610 | WO2016049230 SEQ ID NO: 16 |
| AAVF16/HSC16 | 611 | WO2016049230 SEQ ID NO: 17 |
| AAVF17/HSC17 | 612 | WO2016049230 SEQ ID NO: 13 |
| AAV CBr-E1 | 613 | U.S. Pat. No. 8,734,809 SEQ ID NO: 13 |
| AAV CBr-E2 | 614 | U.S. Pat. No. 8,734,809 SEQ ID NO: 14 |
| AAV CBr-E3 | 615 | U.S. Pat. No. 8,734,809 SEQ ID NO: 15 |
| AAV CBr-E4 | 616 | U.S. Pat. No. 8,734,809 SEQ ID NO: 16 |
| AAV CBr-E5 | 617 | U.S. Pat. No. 8,734,809 SEQ ID NO: 17 |
| AAV CBr-e5 | 618 | U.S. Pat. No. 8,734,809 SEQ ID NO: 18 |
| AAV CBr-E6 | 619 | U.S. Pat. No. 8,734,809 SEQ ID NO: 19 |
| AAV CBr-E7 | 620 | U.S. Pat. No. 8,734,809 SEQ ID NO: 20 |
| AAV CBr-E8 | 621 | U.S. Pat. No. 8,734,809 SEQ ID NO: 21 |
| AAV CLv-D1 | 622 | U.S. Pat. No. 8,734,809 SEQ ID NO: 22 |
| AAV CLv-D2 | 623 | U.S. Pat. No. 8,734,809 SEQ ID NO: 23 |
| AAV CLv-D3 | 624 | U.S. Pat. No. 8,734,809 SEQ ID NO: 24 |
| AAV CLv-D4 | 625 | U.S. Pat. No. 8,734,809 SEQ ID NO: 25 |
| AAV CLv-D5 | 626 | U.S. Pat. No. 8,734,809 SEQ ID NO: 26 |
| AAV CLv-D6 | 627 | U.S. Pat. No. 8,734,809 SEQ ID NO: 27 |
| AAV CLv-D7 | 628 | U.S. Pat. No. 8,734,809 SEQ ID NO: 28 |
| AAV CLv-D8 | 629 | U.S. Pat. No. 8,734,809 SEQ ID NO: 29 |
| AAV CLv-E1 | 630 | U.S. Pat. No. 8,734,809 SEQ ID NO: 13 |
| AAV CLv-R1 | 631 | U.S. Pat. No. 8,734,809 SEQ ID NO: 30 |
| AAV CLv-R2 | 632 | U.S. Pat. No. 8,734,809 SEQ ID NO: 31 |
| AAV CLv-R3 | 633 | U.S. Pat. No. 8,734,809 SEQ ID NO: 32 |
| AAV CLv-R4 | 634 | U.S. Pat. No. 8,734,809 SEQ ID NO: 33 |
| AAV CLv-R5 | 635 | U.S. Pat. No. 8,734,809 SEQ ID NO: 34 |
| AAV CLv-R6 | 636 | U.S. Pat. No. 8,734,809 SEQ ID NO: 35 |
| AAV CLv-R7 | 637 | U.S. Pat. No. 8,734,809 SEQ ID NO: 36 |
| AAV CLv-R8 | 638 | U.S. Pat. No. 8,734,809 SEQ ID NO: 37 |
| AAV CLv-R9 | 639 | U.S. Pat. No. 8,734,809 SEQ ID NO: 38 |
| AAV CLg-F1 | 640 | U.S. Pat. No. 8,734,809 SEQ ID NO: 39 |
| AAV CLg-F2 | 641 | U.S. Pat. No. 8,734,809 SEQ ID NO: 40 |
| AAV CLg-F3 | 642 | U.S. Pat. No. 8,734,809 SEQ ID NO: 41 |
| AAV CLg-F4 | 643 | U.S. Pat. No. 8,734,809 SEQ ID NO: 42 |
| AAV CLg-F5 | 644 | U.S. Pat. No. 8,734,809 SEQ ID NO: 43 |
| AAV CLg-F6 | 645 | U.S. Pat. No. 8,734,809 SEQ ID NO: 43 |
| AAV CLg-F7 | 646 | U.S. Pat. No. 8,734,809 SEQ ID NO: 44 |
| AAV CLg-F8 | 647 | U.S. Pat. No. 8,734,809 SEQ ID NO: 43 |
| AAV CSp-1 | 648 | U.S. Pat. No. 8,734,809 SEQ ID NO: 45 |
| AAV CSp-10 | 649 | U.S. Pat. No. 8,734,809 SEQ ID NO: 46 |
| AAV CSp-11 | 650 | U.S. Pat. No. 8,734,809 SEQ ID NO: 47 |
| AAV CSp-2 | 651 | U.S. Pat. No. 8,734,809 SEQ ID NO: 48 |
| AAV CSp-3 | 652 | U.S. Pat. No. 8,734,809 SEQ ID NO: 49 |
| AAV CSp-4 | 653 | U.S. Pat. No. 8,734,809 SEQ ID NO: 50 |
| AAV CSp-6 | 654 | U.S. Pat. No. 8,734,809 SEQ ID NO: 51 |
| AAV CSp-7 | 655 | U.S. Pat. No. 8,734,809 SEQ ID NO: 52 |
| AAV CSp-8 | 656 | U.S. Pat. No. 8,734,809 SEQ ID NO: 53 |
| AAV CSp-9 | 657 | U.S. Pat. No. 8,734,809 SEQ ID NO: 54 |
| AAV CHt-2 | 658 | U.S. Pat. No. 8,734,809 SEQ ID NO: 55 |
| AAV CHt-3 | 659 | U.S. Pat. No. 8,734,809 SEQ ID NO: 56 |
| AAV CKd-1 | 660 | U.S. Pat. No. 8,734,809 SEQ ID NO: 57 |
| AAV CKd-10 | 661 | U.S. Pat. No. 8,734,809 SEQ ID NO: 58 |
| AAV CKd-2 | 662 | U.S. Pat. No. 8,734,809 SEQ ID NO: 59 |
| AAV CKd-3 | 663 | U.S. Pat. No. 8,734,809 SEQ ID NO: 60 |
| AAV CKd-4 | 664 | U.S. Pat. No. 8,734,809 SEQ ID NO: 61 |
| AAV CKd-6 | 665 | U.S. Pat. No. 8,734,809 SEQ ID NO: 62 |
| AAV CKd-7 | 666 | U.S. Pat. No. 8,734,809 SEQ ID NO: 63 |
| AAV CKd-8 | 667 | U.S. Pat. No. 8,734,809 SEQ ID NO: 64 |
| AAV CLv-1 | 668 | U.S. Pat. No. 8,734,809 SEQ ID NO: 65 |
| AAV CLv-12 | 669 | U.S. Pat. No. 8,734,809 SEQ ID NO: 66 |
| AAV CLv-13 | 670 | U.S. Pat. No. 8,734,809 SEQ ID NO: 67 |
| AAV CLv-2 | 671 | U.S. Pat. No. 8,734,809 SEQ ID NO: 68 |
| AAV CLv-3 | 672 | U.S. Pat. No. 8,734,809 SEQ ID NO: 69 |
| AAV CLv-4 | 673 | U.S. Pat. No. 8,734,809 SEQ ID NO: 70 |
| AAV CLv-6 | 674 | U.S. Pat. No. 8,734,809 SEQ ID NO: 71 |
| AAV CLv-8 | 675 | U.S. Pat. No. 8,734,809 SEQ ID NO: 72 |
| AAV CKd-B1 | 676 | U.S. Pat. No. 8,734,809 SEQ ID NO: 73 |
| AAV CKd-B2 | 677 | U.S. Pat. No. 8,734,809 SEQ ID NO: 74 |
| AAV CKd-B3 | 678 | U.S. Pat. No. 8,734,809 SEQ ID NO: 75 |
| AAV CKd-B4 | 679 | U.S. Pat. No. 8,734,809 SEQ ID NO: 76 |
| AAV CKd-B5 | 680 | U.S. Pat. No. 8,734,809 SEQ ID NO: 77 |
| AAV CKd-B6 | 681 | U.S. Pat. No. 8,734,809 SEQ ID NO: 78 |
| AAV CKd-B7 | 682 | U.S. Pat. No. 8,734,809 SEQ ID NO: 79 |
| AAV CKd-B8 | 683 | U.S. Pat. No. 8,734,809 SEQ ID NO: 80 |
| AAV CKd-H1 | 684 | U.S. Pat. No. 8,734,809 SEQ ID NO: 81 |
| AAV CKd-H2 | 685 | U.S. Pat. No. 8,734,809 SEQ ID NO: 82 |
| AAV CKd-H3 | 686 | U.S. Pat. No. 8,734,809 SEQ ID NO: 83 |
| AAV CKd-H4 | 687 | U.S. Pat. No. 8,734,809 SEQ ID NO: 84 |
| AAV CKd-H5 | 688 | U.S. Pat. No. 8,734,809 SEQ ID NO: 85 |
| AAV CKd-H6 | 689 | U.S. Pat. No. 8,734,809 SEQ ID NO: 77 |
| AAV CHt-1 | 690 | U.S. Pat. No. 8,734,809 SEQ ID NO: 86 |
| AAV CLv1-1 | 691 | U.S. Pat. No. 8,734,809 SEQ ID NO: 171 |
| AAV CLv1-2 | 692 | U.S. Pat. No. 8,734,809 SEQ ID NO: 172 |
| AAV CLv1-3 | 693 | U.S. Pat. No. 8,734,809 SEQ ID NO: 173 |
| AAV CLv1-4 | 694 | U.S. Pat. No. 8,734,809 SEQ ID NO: 174 |
| AAV Clv1-7 | 695 | U.S. Pat. No. 8,734,809 SEQ ID NO: 175 |
| AAV Clv1-8 | 696 | U.S. Pat. No. 8,734,809 SEQ ID NO: 176 |
| AAV Clv 1-9 | 697 | U.S. Pat. No. 8,734,809 SEQ ID NO: 177 |
| AAV Clv1-10 | 698 | U.S. Pat. No. 8,734,809 SEQ ID NO: 178 |
| AAV.VR-355 | 699 | U.S. Pat. No. 8,734,809 SEQ ID NO: 181 |
| AAV.hu.48R3 | 700 | U.S. Pat. No. 8,734,809 SEQ ID NO: 183 |
| AAV CBr-E1 | 701 | U.S. Pat. No. 8,734,809 SEQ ID NO: 87 |
| AAV CBr-E2 | 702 | U.S. Pat. No. 8,734,809 SEQ ID NO: 88 |
| AAV CBr-E3 | 703 | U.S. Pat. No. 8,734,809 SEQ ID NO: 89 |
| AAV CBr-E4 | 704 | U.S. Pat. No. 8,734,809 SEQ ID NO: 90 |
| AAV CBr-E5 | 705 | U.S. Pat. No. 8,734,809 SEQ ID NO: 91 |
| AAV CBr-e5 | 706 | U.S. Pat. No. 8,734,809 SEQ ID NO: 92 |
| AAV CBr-E6 | 707 | U.S. Pat. No. 8,734,809 SEQ ID NO: 93 |
| AAV CBr-E7 | 708 | U.S. Pat. No. 8,734,809 SEQ ID NO: 94 |
| AAV CBr-E8 | 709 | U.S. Pat. No. 8,734,809 SEQ ID NO: 95 |
| AAV CLv-D1 | 710 | U.S. Pat. No. 8,734,809 SEQ ID NO: 96 |
| AAV CLv-D2 | 711 | U.S. Pat. No. 8,734,809 SEQ ID NO: 97 |
| AAV CLv-D3 | 712 | U.S. Pat. No. 8,734,809 SEQ ID NO: 98 |
| AAV CLv-D4 | 713 | U.S. Pat. No. 8,734,809 SEQ ID NO: 99 |
| AAV CLv-D5 | 714 | U.S. Pat. No. 8,734,809 SEQ ID NO: 100 |
| AAV CLv-D6 | 715 | U.S. Pat. No. 8,734,809 SEQ ID NO: 101 |
| AAV CLv-D7 | 716 | U.S. Pat. No. 8,734,809 SEQ ID NO: 102 |
| AAV CLv-D8 | 717 | U.S. Pat. No. 8,734,809 SEQ ID NO: 103 |
| AAV CLv-E1 | 718 | U.S. Pat. No. 8,734,809 SEQ ID NO: 87 |
| AAV CLv-R1 | 719 | U.S. Pat. No. 8,734,809 SEQ ID NO: 104 |
| AAV CLv-R2 | 720 | U.S. Pat. No. 8,734,809 SEQ ID NO: 105 |
| AAV CLv-R3 | 721 | U.S. Pat. No. 8,734,809 SEQ ID NO: 106 |
| AAV CLv-R4 | 722 | U.S. Pat. No. 8,734,809 SEQ ID NO: 107 |
| AAV CLv-R5 | 723 | U.S. Pat. No. 8,734,809 SEQ ID NO: 108 |
| AAV CLv-R6 | 724 | U.S. Pat. No. 8,734,809 SEQ ID NO: 109 |
| AAV CLv-R7 | 725 | U.S. Pat. No. 8,734,809 SEQ ID NO: 110 |
| AAV CLv-R8 | 726 | U.S. Pat. No. 8,734,809 SEQ ID NO: 111 |
| AAV CLv-R9 | 727 | U.S. Pat. No. 8,734,809 SEQ ID NO: 112 |
| AAV CLg-F1 | 728 | U.S. Pat. No. 8,734,809 SEQ ID NO: 113 |
| AAV CLg-F2 | 729 | U.S. Pat. No. 8,734,809 SEQ ID NO: 114 |
| AAV CLg-F3 | 730 | U.S. Pat. No. 8,734,809 SEQ ID NO: 115 |
| AAV CLg-F4 | 731 | U.S. Pat. No. 8,734,809 SEQ ID NO: 116 |
| AAV CLg-F5 | 732 | U.S. Pat. No. 8,734,809 SEQ ID NO: 117 |
| AAV CLg-F6 | 733 | U.S. Pat. No. 8,734,809 SEQ ID NO: 117 |
| AAV CLg-F7 | 734 | U.S. Pat. No. 8,734,809 SEQ ID NO: 118 |
| AAV CLg-F8 | 735 | U.S. Pat. No. 8,734,809 SEQ ID NO: 117 |
| AAV CSp-1 | 736 | U.S. Pat. No. 8,734,809 SEQ ID NO: 119 |
| AAV CSp-10 | 737 | U.S. Pat. No. 8,734,809 SEQ ID NO: 120 |
| AAV CSp-11 | 738 | U.S. Pat. No. 8,734,809 SEQ ID NO: 121 |
| AAV CSp-2 | 739 | U.S. Pat. No. 8,734,809 SEQ ID NO: 122 |
| AAV CSp-3 | 740 | U.S. Pat. No. 8,734,809 SEQ ID NO: 123 |
| AAV CSp-4 | 741 | U.S. Pat. No. 8,734,809 SEQ ID NO: 124 |
| AAV CSp-6 | 742 | U.S. Pat. No. 8,734,809 SEQ ID NO: 125 |
| AAV CSp-7 | 743 | U.S. Pat. No. 8,734,809 SEQ ID NO: 126 |
| AAV CSp-8 | 744 | U.S. Pat. No. 8,734,809 SEQ ID NO: 127 |
| AAV CSp-9 | 745 | U.S. Pat. No. 8,734,809 SEQ ID NO: 128 |
| AAV CHt-2 | 746 | U.S. Pat. No. 8,734,809 SEQ ID NO: 129 |
| AAV CHt-3 | 747 | U.S. Pat. No. 8,734,809 SEQ ID NO: 130 |
| AAV CKd-1 | 748 | U.S. Pat. No. 8,734,809 SEQ ID NO: 131 |
| AAV CKd-10 | 749 | U.S. Pat. No. 8,734,809 SEQ ID NO: 132 |
| AAV CKd-2 | 750 | U.S. Pat. No. 8,734,809 SEQ ID NO: 133 |
| AAV CKd-3 | 751 | U.S. Pat. No. 8,734,809 SEQ ID NO: 134 |
| AAV CKd-4 | 752 | U.S. Pat. No. 8,734,809 SEQ ID NO: 135 |
| AAV CKd-6 | 753 | U.S. Pat. No. 8,734,809 SEQ ID NO: 136 |
| AAV CKd-7 | 754 | U.S. Pat. No. 8,734,809 SEQ ID NO: 137 |
| AAV CKd-8 | 755 | U.S. Pat. No. 8,734,809 SEQ ID NO: 138 |
| AAV CLv-1 | 756 | U.S. Pat. No. 8,734,809 SEQ ID NO: 139 |
| AAV CLv-12 | 757 | U.S. Pat. No. 8,734,809 SEQ ID NO: 140 |
| AAV CLv-13 | 758 | U.S. Pat. No. 8,734,809 SEQ ID NO: 141 |
| AAV CLv-2 | 759 | U.S. Pat. No. 8,734,809 SEQ ID NO: 142 |
| AAV CLv-3 | 760 | U.S. Pat. No. 8,734,809 SEQ ID NO: 143 |
| AAV CLv-4 | 761 | U.S. Pat. No. 8,734,809 SEQ ID NO: 144 |
| AAV CLv-6 | 762 | U.S. Pat. No. 8,734,809 SEQ ID NO: 145 |
| AAV CLv-8 | 763 | U.S. Pat. No. 8,734,809 SEQ ID NO: 146 |
| AAV CKd-B1 | 764 | U.S. Pat. No. 8,734,809 SEQ ID NO: 147 |
| AAV CKd-B2 | 765 | U.S. Pat. No. 8,734,809 SEQ ID NO: 148 |
| AAV CKd-B3 | 766 | U.S. Pat. No. 8,734,809 SEQ ID NO: 149 |
| AAV CKd-B4 | 767 | U.S. Pat. No. 8,734,809 SEQ ID NO: 150 |
| AAV CKd-B5 | 768 | U.S. Pat. No. 8,734,809 SEQ ID NO: 151 |
| AAV CKd-B6 | 769 | U.S. Pat. No. 8,734,809 SEQ ID NO: 152 |
| AAV CKd-B7 | 770 | U.S. Pat. No. 8,734,809 SEQ ID NO: 153 |
| AAV CKd-B8 | 771 | U.S. Pat. No. 8,734,809 SEQ ID NO: 154 |
| AAV CKd-H1 | 772 | U.S. Pat. No. 8,734,809 SEQ ID NO: 155 |
| AAV CKd-H2 | 773 | U.S. Pat. No. 8,734,809 SEQ ID NO: 156 |
| AAV CKd-H3 | 774 | U.S. Pat. No. 8,734,809 SEQ ID NO: 157 |
| AAV CKd-H4 | 775 | U.S. Pat. No. 8,734,809 SEQ ID NO: 158 |
| AAV CKd-H5 | 776 | U.S. Pat. No. 8,734,809 SEQ ID NO: 159 |
| AAV CKd-H6 | 777 | U.S. Pat. No. 8,734,809 SEQ ID NO: 151 |
| AAV CHt-1 | 778 | U.S. Pat. No. 8,734,809 SEQ ID NO: 160 |
| AAV CHt-P2 | 779 | WO2016065001 SEQ ID NO: 1 |
| AAV CHt-P5 | 780 | WO2016065001 SEQ ID NO: 2 |
| AAV CHt-P9 | 781 | WO2016065001 SEQ ID NO: 3 |
| AAV CBr-7.1 | 782 | WO2016065001 SEQ ID NO: 4 |
| AAV CBr-7.2 | 783 | WO2016065001 SEQ ID NO: 5 |
| AAV CBr-7.3 | 784 | WO2016065001 SEQ ID NO: 6 |
| AAV CBr-7.4 | 785 | WO2016065001 SEQ ID NO: 7 |
| AAV CBr-7.5 | 786 | WO2016065001 SEQ ID NO: 8 |
| AAV CBr-7.7 | 787 | WO2016065001 SEQ ID NO: 9 |
| AAV CBr-7.8 | 788 | WO2016065001 SEQ ID NO: 10 |
| AAV CBr-7.10 | 789 | WO2016065001 SEQ ID NO: 11 |
| AAV CKd-N3 | 790 | WO2016065001 SEQ ID NO: 12 |
| AAV CKd-N4 | 791 | WO2016065001 SEQ ID NO: 13 |
| AAV CKd-N9 | 792 | WO2016065001 SEQ ID NO: 14 |
| AAV CLv-L4 | 793 | WO2016065001 SEQ ID NO: 15 |
| AAV CLv-L5 | 794 | WO2016065001 SEQ ID NO: 16 |
| AAV CLv-L6 | 795 | WO2016065001 SEQ ID NO: 17 |
| AAV CLv-K1 | 796 | WO2016065001 SEQ ID NO: 18 |
| AAV CLv-K3 | 797 | WO2016065001 SEQ ID NO: 19 |
| AAV CLv-K6 | 798 | WO2016065001 SEQ ID NO: 20 |
| AAV CLv-M1 | 799 | WO2016065001 SEQ ID NO: 21 |
| AAV CLv-M11 | 800 | WO2016065001 SEQ ID NO: 22 |
| AAV CLv-M2 | 801 | WO2016065001 SEQ ID NO: 23 |
| AAV CLv-M5 | 802 | WO2016065001 SEQ ID NO: 24 |
| AAV CLv-M6 | 803 | WO2016065001 SEQ ID NO: 25 |
| AAV CLv-M7 | 804 | WO2016065001 SEQ ID NO: 26 |
| AAV CLv-M8 | 805 | WO2016065001 SEQ ID NO: 27 |
| AAV CLv-M9 | 806 | WO2016065001 SEQ ID NO: 28 |
| AAV CHt-P1 | 807 | WO2016065001 SEQ ID NO: 29 |
| AAV CHt-P6 | 808 | WO2016065001 SEQ ID NO: 30 |
| AAV CHt-P8 | 809 | WO2016065001 SEQ ID NO: 31 |
| AAV CHt-6.1 | 810 | WO2016065001 SEQ ID NO: 32 |
| AAV CHt-6.10 | 811 | WO2016065001 SEQ ID NO: 33 |
| AAV CHt-6.5 | 812 | WO2016065001 SEQ ID NO: 34 |
| AAV CHt-6.6 | 813 | WO2016065001 SEQ ID NO: 35 |
| AAV CHt-6.7 | 814 | WO2016065001 SEQ ID NO: 36 |
| AAV CHt-6.8 | 815 | WO2016065001 SEQ ID NO: 37 |
| AAV CSp-8.10 | 816 | WO2016065001 SEQ ID NO: 38 |
| AAV CSp-8.2 | 817 | WO2016065001 SEQ ID NO: 39 |
| AAV CSp-8.4 | 818 | WO2016065001 SEQ ID NO: 40 |
| AAV CSp-8.5 | 819 | WO2016065001 SEQ ID NO: 41 |
| AAV CSp-8.6 | 820 | WO2016065001 SEQ ID NO: 42 |
| AAV CSp-8.7 | 821 | WO2016065001 SEQ ID NO: 43 |
| AAV CSp-8.8 | 822 | WO2016065001 SEQ ID NO: 44 |
| AAV CSp-8.9 | 823 | WO2016065001 SEQ ID NO: 45 |
| AAV CBr-B7.3 | 824 | WO2016065001 SEQ ID NO: 46 |
| AAV CBr-B7.4 | 825 | WO2016065001 SEQ ID NO: 47 |
| AAV3B | 826 | WO2016065001 SEQ ID NO: 48 |
| AAV4 | 827 | WO2016065001 SEQ ID NO: 49 |
| AAV5 | 828 | WO2016065001 SEQ ID NO: 50 |
| AAV CHt-P2 | 829 | WO2016065001 SEQ ID NO: 51 |
| AAV CHt-P5 | 830 | WO2016065001 SEQ ID NO: 52 |
| AAV CHt-P9 | 831 | WO2016065001 SEQ ID NO: 53 |
| AAV CBr-7.1 | 832 | WO2016065001 SEQ ID NO: 54 |
| AAV CBr-7.2 | 833 | WO2016065001 SEQ ID NO: 55 |
| AAV CBr-7.3 | 834 | WO2016065001 SEQ ID NO: 56 |
| AAV CBr-7.4 | 835 | WO2016065001 SEQ ID NO: 57 |
| AAV CBr-7.5 | 836 | WO2016065001 SEQ ID NO: 58 |
| AAV CBr-7.7 | 837 | WO2016065001 SEQ ID NO: 59 |
| AAV CBr-7.8 | 838 | WO2016065001 SEQ ID NO: 60 |
| AAV CBr-7.10 | 839 | WO2016065001 SEQ ID NO: 61 |
| AAV CKd-N3 | 840 | WO2016065001 SEQ ID NO: 62 |
| AAV CKd-N4 | 841 | WO2016065001 SEQ ID NO: 63 |
| AAV CKd-N9 | 842 | WO2016065001 SEQ ID NO: 64 |
| AAV CLv-L4 | 843 | WO2016065001 SEQ ID NO: 65 |
| AAV CLv-L5 | 844 | WO2016065001 SEQ ID NO: 66 |
| AAV CLv-L6 | 845 | WO2016065001 SEQ ID NO: 67 |
| AAV CLv-K1 | 846 | WO2016065001 SEQ ID NO: 68 |
| AAV CLv-K3 | 847 | WO2016065001 SEQ ID NO: 69 |
| AAV CLv-K6 | 848 | WO2016065001 SEQ ID NO: 70 |
| AAV CLv-M1 | 849 | WO2016065001 SEQ ID NO: 71 |
| AAV CLv-M11 | 850 | WO2016065001 SEQ ID NO: 72 |
| AAV CLv-M2 | 851 | WO2016065001 SEQ ID NO: 73 |
| AAV CLv-M5 | 852 | WO2016065001 SEQ ID NO: 74 |
| AAV CLv-M6 | 853 | WO2016065001 SEQ ID NO: 75 |
| AAV CLv-M7 | 854 | WO2016065001 SEQ ID NO: 76 |
| AAV CLv-M8 | 855 | WO2016065001 SEQ ID NO: 77 |
| AAV CLv-M9 | 856 | WO2016065001 SEQ ID NO: 78 |
| AAV CHt-P1 | 857 | WO2016065001 SEQ ID NO: 79 |
| AAV CHt-P6 | 858 | WO2016065001 SEQ ID NO: 80 |
| AAV CHt-P8 | 859 | WO2016065001 SEQ ID NO: 81 |
| AAV CHt-6.1 | 860 | WO2016065001 SEQ ID NO: 82 |
| AAV CHt-6.10 | 861 | WO2016065001 SEQ ID NO: 83 |
| AAV CHt-6.5 | 862 | WO2016065001 SEQ ID NO: 84 |
| AAV CHt-6.6 | 863 | WO2016065001 SEQ ID NO: 85 |
| AAV CHt-6.7 | 864 | WO2016065001 SEQ ID NO: 86 |
| AAV CHt-6.8 | 865 | WO2016065001 SEQ ID NO: 87 |
| AAV CSp-8.10 | 866 | WO2016065001 SEQ ID NO: 88 |
| AAV CSp-8.2 | 867 | WO2016065001 SEQ ID NO: 89 |
| AAV CSp-8.4 | 868 | WO2016065001 SEQ ID NO: 90 |
| AAV CSp-8.5 | 869 | WO2016065001 SEQ ID NO: 91 |
| AAV CSp-8.6 | 870 | WO2016065001 SEQ ID NO: 92 |
| AAV CSp-8.7 | 871 | WO2016065001 SEQ ID NO: 93 |
| AAV CSp-8.8 | 872 | WO2016065001 SEQ ID NO: 94 |
| AAV CSp-8.9 | 873 | WO2016065001 SEQ ID NO: 95 |
| AAV CBr-B7.3 | 874 | WO2016065001 SEQ ID NO: 96 |
| AAV CBr-B7.4 | 875 | WO2016065001 SEQ ID NO: 97 |
| AAV3B | 876 | WO2016065001 SEQ ID NO: 98 |
| AAV4 | 877 | WO2016065001 SEQ ID NO: 99 |
| AAV5 | 878 | WO2016065001 SEQ ID NO: 100 |
| GPV | 879 | U.S. Pat. No. 9,624,274B2 SEQ ID NO: 192 |
| B19 | 880 | U.S. Pat. No. 9,624,274B2 SEQ ID NO: 193 |
| MVM | 881 | U.S. Pat. No. 9,624,274B2 SEQ ID NO: 194 |
| FPV | 882 | U.S. Pat. No. 9,624,274B2 SEQ ID NO: 195 |
| CPV | 883 | U.S. Pat. No. 9,624,274B2 SEQ ID NO: 196 |
| AAV6 | 884 | U.S. Pat. No. 9,546,112B2 SEQ ID NO: 5 |
| AAV6 | 885 | U.S. Pat. No. 9,457,103B2 SEQ ID NO: 1 |
| AAV2 | 886 | U.S. Pat. No. 9,457,103B2 SEQ ID NO: 2 |
| ShH10 | 887 | U.S. Pat. No. 9,457,103B2 SEQ ID NO: 3 |
| ShH13 | 888 | U.S. Pat. No. 9,457,103B2 SEQ ID NO: 4 |
| ShH10 | 889 | U.S. Pat. No. 9,457,103B2 SEQ ID NO: 5 |
| ShH10 | 890 | U.S. Pat. No. 9,457,103B2 SEQ ID NO: 6 |
| ShH10 | 891 | U.S. Pat. No. 9,457,103B2 SEQ ID NO: 7 |
| ShH10 | 892 | U.S. Pat. No. 9,457,103B2 SEQ ID NO: 8 |
| ShH10 | 893 | U.S. Pat. No. 9,457,103B2 SEQ ID NO: 9 |
| rh74 | 894 | U.S. Pat. No. 9,434,928B2 SEQ ID NO: 1, US2015023924A1 SEQ ID NO: 2 |
| rh74 | 895 | U.S. Pat. No. 9,434,928B2 SEQ ID NO: 2, US2015023924A1 SEQ ID NO: 1 |
| AAV8 | 896 | U.S. Pat. No. 9,434,928B2 SEQ ID NO: 4 |
| rh74 | 897 | U.S. Pat. No. 9,434,928B2 SEQ ID NO: 5 |
| rh74 (RHM4-1) | 898 | US2015023924A1 SEQ ID NO: 5, US20160375110A1 SEQ ID NO: 4 |
| rh74 (RHM15-1) | 899 | US2015023924A1 SEQ ID NO: 6, US20160375110A1 SEQ ID NO: 5 |
| rh74 (RHM15-2) | 900 | US2015023924A1 SEQ ID NO: 7, US20160375110A1 SEQ ID NO: 6 |
| rh74 (RHM15-3/RHM15-5) | 901 | US2015023924A1 SEQ ID NO: 8, US20160375110A1 SEQ ID NO: 7 |
| rh74 (RHM15-4) | 902 | US2015023924A1 SEQ ID NO: 9, US20160375110A1 SEQ ID NO: 8 |
| rh74 (RHM15-6) | 903 | US2015023924A1 SEQ ID NO: 10, US20160375110A1 SEQ ID NO: 9 |
| rh74 (RHM4-1) | 904 | US2015023924A1 SEQ ID NO: 11 |
| rh74 (RHM15-1) | 905 | US2015023924A1 SEQ ID NO: 12 |
| rh74 (RHM15-2) | 906 | US2015023924A1 SEQ ID NO: 13 |
| rh74 (RHM15-3/RHM15-5) | 907 | US2015023924A1 SEQ ID NO: 14 |
| rh74 (RHM15-4) | 908 | US2015023924A1 SEQ ID NO: 15 |
| rh74 (RHM15-6) | 909 | US2015023924A1 SEQ ID NO: 16 |
| AAV2 (comprising lung | 910 | US20160175389A1 SEQ ID NO: 9 |
| specific polypeptide) | | |
| AAV2 (comprising lung | 911 | US20160175389A1 SEQ ID NO: 10 |
| specific polypeptide) | | |
| Anc80 | 912 | US20170051257A1 SEQ ID NO: 1 |
| Anc80 | 913 | US20170051257A1 SEQ ID NO: 2 |
| Anc81 | 914 | US20170051257A1 SEQ ID NO: 3 |
| Anc80 | 915 | US20170051257A1 SEQ ID NO: 4 |
| Anc82 | 916 | US20170051257A1 SEQ ID NO: 5 |
| Anc82 | 917 | US20170051257A1 SEQ ID NO: 6 |
| Anc83 | 918 | US20170051257A1 SEQ ID NO: 7 |
| Anc83 | 919 | US20170051257A1 SEQ ID NO: 8 |
| Anc84 | 920 | US20170051257A1 SEQ ID NO: 9 |
| Anc84 | 921 | US20170051257A1 SEQ ID NO: 10 |
| Anc94 | 922 | US20170051257A1 SEQ ID NO: 11 |
| Anc94 | 923 | US20170051257A1 SEQ ID NO: 12 |
| Anc113 | 924 | US20170051257A1 SEQ ID NO: 13 |
| Anc113 | 925 | US20170051257A1 SEQ ID NO: 14 |
| Anc126 | 926 | US20170051257A1 SEQ ID NO: 15 |
| Anc126 | 927 | US20170051257A1 SEQ ID NO: 16 |
| Anc127 | 928 | US20170051257A1 SEQ ID NO: 17 |
| Anc127 | 929 | US20170051257A1 SEQ ID NO: 18 |
| Anc80L27 | 930 | US20170051257A1 SEQ ID NO: 19 |
| Anc80L59 | 931 | US20170051257A1 SEQ ID NO: 20 |
| Anc80L60 | 932 | US20170051257A1 SEQ ID NO: 21 |
| Anc80L62 | 933 | US20170051257A1 SEQ ID NO: 22 |
| Anc80L65 | 934 | US20170051257A1 SEQ ID NO: 23 |
| Anc80L33 | 935 | US20170051257A1 SEQ ID NO: 24 |
| Anc80L36 | 936 | US20170051257A1 SEQ ID NO: 25 |
| Anc80L44 | 937 | US20170051257A1 SEQ ID NO: 26 |
| Anc80L1 | 938 | US20170051257A1 SEQ ID NO: 35 |
| Anc80L1 | 939 | US20170051257A1 SEQ ID NO: 36 |
| AAV-X1 | 940 | U.S. Pat. No. 8,283,151B2 SEQ ID NO: 11 |
| AAV-X1b | 941 | U.S. Pat. No. 8,283,151B2 SEQ ID NO: 12 |
| AAV-X5 | 942 | U.S. Pat. No. 8,283,151B2 SEQ ID NO: 13 |
| AAV-X19 | 943 | U.S. Pat. No. 8,283,151B2 SEQ ID NO: 14 |
| AAV-X21 | 944 | U.S. Pat. No. 8,283,151B2 SEQ ID NO: 15 |
| AAV-X22 | 945 | U.S. Pat. No. 8,283,151B2 SEQ ID NO: 16 |
| AAV-X23 | 946 | U.S. Pat. No. 8,283,151B2 SEQ ID NO: 17 |
| AAV-X24 | 947 | U.S. Pat. No. 8,283,151B2 SEQ ID NO: 18 |
| AAV-X25 | 948 | U.S. Pat. No. 8,283,151B2 SEQ ID NO: 19 |
| AAV-X26 | 949 | U.S. Pat. No. 8,283,151B2 SEQ ID NO: 20 |
| AAV-X1 | 950 | U.S. Pat. No. 8,283,151B2 SEQ ID NO: 21 |
| AAV-X1b | 951 | U.S. Pat. No. 8,283,151B2 SEQ ID NO: 22 |
| AAV-X5 | 952 | U.S. Pat. No. 8,283,151B2 SEQ ID NO: 23 |
| AAV-X19 | 953 | U.S. Pat. No. 8,283,151B2 SEQ ID NO: 24 |
| AAV-X21 | 954 | U.S. Pat. No. 8,283,151B2 SEQ ID NO: 25 |
| AAV-X22 | 955 | U.S. Pat. No. 8,283,151B2 SEQ ID NO: 26 |
| AAV-X23 | 956 | U.S. Pat. No. 8,283,151B2 SEQ ID NO: 27 |
| AAV-X24 | 957 | U.S. Pat. No. 8,283,151B2 SEQ ID NO: 28 |
| AAV-X25 | 958 | U.S. Pat. No. 8,283,151B2 SEQ ID NO: 29 |
| AAV-X26 | 959 | U.S. Pat. No. 8,283,151B2 SEQ ID NO: 30 |
| AAVrh8 | 960 | WO2016054554A1 SEQ ID NO: 8 |
| AAVrh8VP2FC5 | 961 | WO2016054554A1 SEQ ID NO: 9 |
| AAVrh8VP2FC44 | 962 | WO2016054554A1 SEQ ID NO: 10 |
| AAVrh8VP2ApoB100 | 963 | WO2016054554A1 SEQ ID NO: 11 |
| AAVrh8VP2RVG | 964 | WO2016054554A1 SEQ ID NO: 12 |
| AAVrh8VP2Angiopep-2 VP2 | 965 | WO2016054554A1 SEQ ID NO: 13 |
| AAV9.47VP1.3 | 966 | WO2016054554A1 SEQ ID NO: 14 |
| AAV9.47VP2ICAMg3 | 967 | WO2016054554A1 SEQ ID NO: 15 |
| AAV9.47VP2RVG | 968 | WO2016054554A1 SEQ ID NO: 16 |
| AAV9.47VP2Angiopep-2 | 969 | WO2016054554A1 SEQ ID NO: 17 |
| AAV9.47VP2A-string | 970 | WO2016054554A1 SEQ ID NO: 18 |
| AAVrh8VP2FC5 VP2 | 971 | WO2016054554A1 SEQ ID NO: 19 |
| AAVrh8VP2FC44 VP2 | 972 | WO2016054554A1 SEQ ID NO: 20 |
| AAVrh8VP2ApoB100 VP2 | 973 | WO2016054554A1 SEQ ID NO: 21 |
| AAVrh8VP2RVG VP2 | 974 | WO2016054554A1 SEQ ID NO: 22 |
| AAVrh8VP2Angiopep-2 VP2 | 975 | WO2016054554A1 SEQ ID NO: 23 |
| AAV9.47VP2ICAMg3 VP2 | 976 | WO2016054554A1 SEQ ID NO: 24 |
| AAV9.47VP2RVG VP2 | 977 | WO2016054554A1 SEQ ID NO: 25 |
| AAV9.47VP2Angiopep-2 VP2 | 978 | WO2016054554A1 SEQ ID NO: 26 |
| AAV9.47VP2A-string VP2 | 979 | WO2016054554A1 SEQ ID NO: 27 |
| rAAV-B1 | 980 | WO2016054557A1 SEQ ID NO: 1 |
| rAAV-B2 | 981 | WO2016054557A1 SEQ ID NO: 2 |
| rAAV-B3 | 982 | WO2016054557A1 SEQ ID NO: 3 |
| rAAV-B4 | 983 | WO2016054557A1 SEQ ID NO: 4 |
| rAAV-B1 | 984 | WO2016054557A1 SEQ ID NO: 5 |
| rAAV-B2 | 985 | WO2016054557A1 SEQ ID NO: 6 |
| rAAV-B3 | 986 | WO2016054557A1 SEQ ID NO: 7 |
| rAAV-B4 | 987 | WO2016054557A1 SEQ ID NO: 8 |
| rAAV-L1 | 988 | WO2016054557A1 SEQ ID NO: 9 |
| rAAV-L2 | 989 | WO2016054557A1 SEQ ID NO: 10 |
| rAAV-L3 | 990 | WO2016054557A1 SEQ ID NO: 11 |
| rAAV-L4 | 991 | WO2016054557A1 SEQ ID NO: 12 |
| rAAV-L1 | 992 | WO2016054557A1 SEQ ID NO: 13 |
| rAAV-L2 | 993 | WO2016054557A1 SEQ ID NO: 14 |
| rAAV-L3 | 994 | WO2016054557A1 SEQ ID NO: 15 |
| rAAV-L4 | 995 | WO2016054557A1 SEQ ID NO: 16 |
| AAV9 | 996 | WO2016073739A1 SEQ ID NO: 3 |
| rAAV | 997 | WO2016081811A1 SEQ ID NO: 1 |
| rAAV | 998 | WO2016081811A1 SEQ ID NO: 2 |
| rAAV | 999 | WO2016081811A1 SEQ ID NO: 3 |
| rAAV | 1000 | WO2016081811A1 SEQ ID NO: 4 |
| rAAV | 1001 | WO2016081811A1 SEQ ID NO: 5 |
| rAAV | 1002 | WO2016081811A1 SEQ ID NO: 6 |
| rAAV | 1003 | WO2016081811A1 SEQ ID NO: 7 |
| rAAV | 1004 | WO2016081811A1 SEQ ID NO: 8 |
| rAAV | 1005 | WO2016081811A1 SEQ ID NO: 9 |
| rAAV | 1006 | WO2016081811A1 SEQ ID NO: 10 |
| rAAV | 1007 | WO2016081811A1 SEQ ID NO: 11 |
| rAAV | 1008 | WO2016081811A1 SEQ ID NO: 12 |
| rAAV | 1009 | WO2016081811A1 SEQ ID NO: 13 |
| rAAV | 1010 | WO2016081811A1 SEQ ID NO: 14 |
| rAAV | 1011 | WO2016081811A1 SEQ ID NO: 15 |
| rAAV | 1012 | WO2016081811A1 SEQ ID NO: 16 |
| rAAV | 1013 | WO2016081811A1 SEQ ID NO: 17 |
| rAAV | 1014 | WO2016081811A1 SEQ ID NO: 18 |
| rAAV | 1015 | WO2016081811A1 SEQ ID NO: 19 |
| rAAV | 1016 | WO2016081811A1 SEQ ID NO: 20 |
| rAAV | 1017 | WO2016081811A1 SEQ ID NO: 21 |
| rAAV | 1018 | WO2016081811A1 SEQ ID NO: 22 |
| rAAV | 1019 | WO2016081811A1 SEQ ID NO: 23 |
| rAAV | 1020 | WO2016081811A1 SEQ ID NO: 24 |
| rAAV | 1021 | WO2016081811A1 SEQ ID NO: 25 |
| rAAV | 1022 | WO2016081811A1 SEQ ID NO: 26 |
| rAAV | 1023 | WO2016081811A1 SEQ ID NO: 27 |
| rAAV | 1024 | WO2016081811A1 SEQ ID NO: 28 |
| rAAV | 1025 | WO2016081811A1 SEQ ID NO: 29 |
| rAAV | 1026 | WO2016081811A1 SEQ ID NO: 30 |
| rAAV | 1027 | WO2016081811A1 SEQ ID NO: 31 |
| rAAV | 1028 | WO2016081811A1 SEQ ID NO: 32 |
| rAAV | 1029 | WO2016081811A1 SEQ ID NO: 33 |
| rAAV | 1030 | WO2016081811A1 SEQ ID NO: 34 |
| rAAV | 1031 | WO2016081811A1 SEQ ID NO: 35 |
| rAAV | 1032 | WO2016081811A1 SEQ ID NO: 36 |
| rAAV | 1033 | WO2016081811A1 SEQ ID NO: 37 |
| rAAV | 1034 | WO2016081811A1 SEQ ID NO: 38 |
| rAAV | 1035 | WO2016081811A1 SEQ ID NO: 39 |
| rAAV | 1036 | WO2016081811A1 SEQ ID NO: 40 |
| rAAV | 1037 | WO2016081811A1 SEQ ID NO: 41 |
| rAAV | 1038 | WO2016081811A1 SEQ ID NO: 42 |
| rAAV | 1039 | WO2016081811A1 SEQ ID NO: 43 |
| rAAV | 1040 | WO2016081811A1 SEQ ID NO: 44 |
| rAAV | 1041 | WO2016081811A1 SEQ ID NO: 45 |
| rAAV | 1042 | WO2016081811A1 SEQ ID NO: 46 |
| rAAV | 1043 | WO2016081811A1 SEQ ID NO: 47 |
| rAAV | 1044 | WO2016081811A1 SEQ ID NO: 48 |
| rAAV | 1045 | WO2016081811A1 SEQ ID NO: 49 |
| rAAV | 1046 | WO2016081811A1 SEQ ID NO: 50 |
| rAAV | 1047 | WO2016081811A1 SEQ ID NO: 51 |
| rAAV | 1048 | WO2016081811A1 SEQ ID NO: 52 |
| rAAV | 1049 | WO2016081811A1 SEQ ID NO: 53 |
| rAAV | 1050 | WO2016081811A1 SEQ ID NO: 54 |
| rAAV | 1051 | WO2016081811A1 SEQ ID NO: 55 |
| rAAV | 1052 | WO2016081811A1 SEQ ID NO: 56 |
| rAAV | 1053 | WO2016081811A1 SEQ ID NO: 57 |
| rAAV | 1054 | WO2016081811A1 SEQ ID NO: 58 |
| rAAV | 1055 | WO2016081811A1 SEQ ID NO: 59 |
| rAAV | 1056 | WO2016081811A1 SEQ ID NO: 60 |
| rAAV | 1057 | WO2016081811A1 SEQ ID NO: 61 |
| rAAV | 1058 | WO2016081811A1 SEQ ID NO: 62 |
| rAAV | 1059 | WO2016081811A1 SEQ ID NO: 63 |
| rAAV | 1060 | WO2016081811A1 SEQ ID NO: 64 |
| rAAV | 1061 | WO2016081811A1 SEQ ID NO: 65 |
| rAAV | 1062 | WO2016081811A1 SEQ ID NO: 66 |
| rAAV | 1063 | WO2016081811A1 SEQ ID NO: 67 |
| rAAV | 1064 | WO2016081811A1 SEQ ID NO: 68 |
| rAAV | 1065 | WO2016081811A1 SEQ ID NO: 69 |
| rAAV | 1066 | WO2016081811A1 SEQ ID NO: 70 |
| rAAV | 1067 | WO2016081811A1 SEQ ID NO: 71 |
| rAAV | 1068 | WO2016081811A1 SEQ ID NO: 72 |
| rAAV | 1069 | WO2016081811A1 SEQ ID NO: 73 |
| rAAV | 1070 | WO2016081811A1 SEQ ID NO: 74 |
| rAAV | 1071 | WO2016081811A1 SEQ ID NO: 75 |
| rAAV | 1072 | WO2016081811A1 SEQ ID NO: 76 |
| rAAV | 1073 | WO2016081811A1 SEQ ID NO: 77 |
| rAAV | 1074 | WO2016081811A1 SEQ ID NO: 78 |
| rAAV | 1075 | WO2016081811A1 SEQ ID NO: 79 |
| rAAV | 1076 | WO2016081811A1 SEQ ID NO: 80 |
| rAAV | 1077 | WO2016081811A1 SEQ ID NO: 81 |
| rAAV | 1078 | WO2016081811A1 SEQ ID NO: 82 |
| rAAV | 1079 | WO2016081811A1 SEQ ID NO: 83 |
| rAAV | 1080 | WO2016081811A1 SEQ ID NO: 84 |
| rAAV | 1081 | WO2016081811A1 SEQ ID NO: 85 |
| rAAV | 1082 | WO2016081811A1 SEQ ID NO: 86 |
| rAAV | 1083 | WO2016081811A1 SEQ ID NO: 87 |
| rAAV | 1084 | WO2016081811A1 SEQ ID NO: 88 |
| rAAV | 1085 | WO2016081811A1 SEQ ID NO: 89 |
| rAAV | 1086 | WO2016081811A1 SEQ ID NO: 90 |
| rAAV | 1087 | WO2016081811A1 SEQ ID NO: 91 |
| rAAV | 1088 | WO2016081811A1 SEQ ID NO: 92 |
| rAAV | 1089 | WO2016081811A1 SEQ ID NO: 93 |
| rAAV | 1090 | WO2016081811A1 SEQ ID NO: 94 |
| rAAV | 1091 | WO2016081811A1 SEQ ID NO: 95 |
| rAAV | 1092 | WO2016081811A1 SEQ ID NO: 96 |
| rAAV | 1093 | WO2016081811A1 SEQ ID NO: 97 |
| rAAV | 1094 | WO2016081811A1 SEQ ID NO: 98 |
| rAAV | 1095 | WO2016081811A1 SEQ ID NO: 99 |
| rAAV | 1096 | WO2016081811A1 SEQ ID NO: 100 |
| rAAV | 1097 | WO2016081811A1 SEQ ID NO: 101 |
| rAAV | 1098 | WO2016081811A1 SEQ ID NO: 102 |
| rAAV | 1099 | WO2016081811A1 SEQ ID NO: 103 |
| rAAV | 1100 | WO2016081811A1 SEQ ID NO: 104 |
| rAAV | 1101 | WO2016081811A1 SEQ ID NO: 105 |
| rAAV | 1102 | WO2016081811A1 SEQ ID NO: 106 |
| rAAV | 1103 | WO2016081811A1 SEQ ID NO: 107 |
| rAAV | 1104 | WO2016081811A1 SEQ ID NO: 108 |
| rAAV | 1105 | WO2016081811A1 SEQ ID NO: 109 |
| rAAV | 1106 | WO2016081811A1 SEQ ID NO: 110 |
| rAAV | 1107 | WO2016081811A1 SEQ ID NO: 111 |
| rAAV | 1108 | WO2016081811A1 SEQ ID NO: 112 |
| rAAV | 1109 | WO2016081811A1 SEQ ID NO: 113 |
| rAAV | 1110 | WO2016081811A1 SEQ ID NO: 114 |
| rAAV | 1111 | WO2016081811A1 SEQ ID NO: 115 |
| rAAV | 1112 | WO2016081811A1 SEQ ID NO: 116 |
| rAAV | 1113 | WO2016081811A1 SEQ ID NO: 117 |
| rAAV | 1114 | WO2016081811A1 SEQ ID NO: 118 |
| rAAV | 1115 | WO2016081811A1 SEQ ID NO: 119 |
| rAAV | 1116 | WO2016081811A1 SEQ ID NO: 120 |
| rAAV | 1117 | WO2016081811A1 SEQ ID NO: 121 |
| rAAV | 1118 | WO2016081811A1 SEQ ID NO: 122 |
| rAAV | 1119 | WO2016081811A1 SEQ ID NO: 123 |
| rAAV | 1120 | WO2016081811A1 SEQ ID NO: 124 |
| rAAV | 1121 | WO2016081811A1 SEQ ID NO: 125 |
| rAAV | 1122 | WO2016081811A1 SEQ ID NO: 126 |
| rAAV | 1123 | WO2016081811A1 SEQ ID NO: 127 |
| rAAV | 1124 | WO2016081811A1 SEQ ID NO: 128 |
| AAV8 E532K | 1125 | WO2016081811A1 SEQ ID NO: 133 |
| AAV8 E532K | 1126 | WO2016081811A1 SEQ ID NO: 134 |
| rAAV4 | 1127 | WO2016115382A1 SEQ ID NO: 2 |
| rAAV4 | 1128 | WO2016115382A1 SEQ ID NO: 3 |
| rAAV4 | 1129 | WO2016115382A1 SEQ ID NO: 4 |
| rAAV4 | 1130 | WO2016115382A1 SEQ ID NO: 5 |
| rAAV4 | 1131 | WO2016115382A1 SEQ ID NO: 6 |
| rAAV4 | 1132 | WO2016115382A1 SEQ ID NO: 7 |
| rAAV4 | 1133 | WO2016115382A1 SEQ ID NO: 8 |
| rAAV4 | 1134 | WO2016115382A1 SEQ ID NO: 9 |
| rAAV4 | 1135 | WO2016115382A1 SEQ ID NO: 10 |
| rAAV4 | 1136 | WO2016115382A1 SEQ ID NO: 11 |
| rAAV4 | 1137 | WO2016115382A1 SEQ ID NO: 12 |
| rAAV4 | 1138 | WO2016115382A1 SEQ ID NO: 13 |
| rAAV4 | 1139 | WO2016115382A1 SEQ ID NO: 14 |
| rAAV4 | 1140 | WO2016115382A1 SEQ ID NO: 15 |
| rAAV4 | 1141 | WO2016115382A1 SEQ ID NO: 16 |
| rAAV4 | 1142 | WO2016115382A1 SEQ ID NO: 17 |
| rAAV4 | 1143 | WO2016115382A1 SEQ ID NO: 18 |
| rAAV4 | 1144 | WO2016115382A1 SEQ ID NO: 19 |
| rAAV4 | 1145 | WO2016115382A1 SEQ ID NO: 20 |
| rAAV4 | 1146 | WO2016115382A1 SEQ ID NO: 21 |
| AAV11 | 1147 | WO2016115382A1 SEQ ID NO: 22 |
| AAV12 | 1148 | WO2016115382A1 SEQ ID NO: 23 |
| rh32 | 1149 | WO2016115382A1 SEQ ID NO: 25 |
| rh33 | 1150 | WO2016115382A1 SEQ ID NO: 26 |
| rh34 | 1151 | WO2016115382A1 SEQ ID NO: 27 |
| rAAV4 | 1152 | WO2016115382A1 SEQ ID NO: 28 |
| rAAV4 | 1153 | WO2016115382A1 SEQ ID NO: 29 |
| rAAV4 | 1154 | WO2016115382A1 SEQ ID NO: 30 |
| rAAV4 | 1155 | WO2016115382A1 SEQ ID NO: 31 |
| rAAV4 | 1156 | WO2016115382A1 SEQ ID NO: 32 |
| rAAV4 | 1157 | WO2016115382A1 SEQ ID NO: 33 |
| AAV2/8 | 1158 | WO2016131981A1 SEQ ID NO: 47 |
| AAV2/8 | 1159 | WO2016131981A1 SEQ ID NO: 48 |
| ancestral AAV | 1160 | WO2016154344A1 SEQ ID NO: 7 |
| ancestral AAV variant C4 | 1161 | WO2016154344A1 SEQ ID NO: 13 |
| ancestral AAV variant C7 | 1162 | WO2016154344A1 SEQ ID NO: 14 |
| ancestral AAV variant G4 | 1163 | WO2016154344A1 SEQ ID NO: 15 |
| consensus amino acid sequence | 1164 | WO2016154344A1 SEQ ID NO: 16 |
| of ancestral AAV variants, C4, | | |
| C7 and G4 | | |
| consensus amino acid sequence | 1165 | WO2016154344A1 SEQ ID NO: 17 |
| of ancestral AAV variants, C4 | | |
| and C7 | | |
| AAV8 (with a AAV2 | 1166 | WO2016150403A1 SEQ ID NO: 13 |
| phospholipase domain) | | |
| AAV VR-942n | 1167 | US20160289275A1 SEQ ID NO: 10 |
| AAV5-A (M569V) | 1168 | US20160289275A1 SEQ ID NO: 13 |
| AAV5-A (M569V) | 1169 | US20160289275A1 SEQ ID NO: 14 |
| AAV5-A (Y585V) | 1170 | US20160289275A1 SEQ ID NO: 16 |
| AAV5-A (Y585V) | 1171 | US20160289275A1 SEQ ID NO: 17 |
| AAV5-A (L587T) | 1172 | US20160289275A1 SEQ ID NO: 19 |
| AAV5-A (L587T) | 1173 | US20160289275A1 SEQ ID NO: 20 |
| AAV5-A (Y585V/L587T) | 1174 | US20160289275A1 SEQ ID NO: 22 |
| AAV5-A (Y585V7L587T) | 1175 | US20160289275A1 SEQ ID NO: 23 |
| AAV5-B (D652A) | 1176 | US20160289275A1 SEQ ID NO: 25 |
| AAV5-B (D652A) | 1177 | US20160289275A1 SEQ ID NO: 26 |
| AAV5-B (T362M) | 1178 | US20160289275A1 SEQ ID NO: 28 |
| AAV5-B (T362M) | 1179 | US20160289275A1 SEQ ID NO: 29 |
| AAV5-B (Q359D) | 1180 | US20160289275A1 SEQ ID NO: 31 |
| AAV5-B (Q359D) | 1181 | US20160289275A1 SEQ ID NO: 32 |
| AAV5-B (E350Q) | 1182 | US20160289275A1 SEQ ID NO: 34 |
| AAV5-B (E350Q) | 1183 | US20160289275A1 SEQ ID NO: 35 |
| AAV5-B (P533S) | 1184 | US20160289275A1 SEQ ID NO: 37 |
| AAV5-B (P533S) | 1185 | US20160289275A1 SEQ ID NO: 38 |
| AAV5-B (P533G) | 1186 | US20160289275A1 SEQ ID NO: 40 |
| AAV5-B (P533G) | 1187 | US20160289275A1 SEQ ID NO: 41 |
| AAV5-mutation in loop VII | 1188 | US20160289275A1 SEQ ID NO: 43 |
| AAV5 -mutation in loop VII | 1189 | US20160289275A1 SEQ ID NO: 44 |
| AAV8 | 1190 | US20160289275A1 SEQ ID NO: 47 |
| Mut A (LK03/AAV8) | 1191 | WO2016181123A1 SEQ ID NO: 1 |
| Mut B (LK03/AAV5) | 1192 | WO2016181123A1 SEQ ID NO: 2 |
| Mut C (AAV8/AAV3B) | 1193 | WO2016181123A1 SEQ ID NO: 3 |
| Mut D (AAV5/AAV3B) | 1194 | WO2016181123A1 SEQ ID NO: 4 |
| Mut E (AAV8/AAV3B) | 1195 | WO2016181123A1 SEQ ID NO: 5 |
| Mut F (AAV3B/AAV8) | 1196 | WO2016181123A1 SEQ ID NO: 6 |
| AAV44.9 | 1197 | WO2016183297A1 SEQ ID NO: 4 |
| AAV44.9 | 1198 | WO2016183297A1 SEQ ID NO: 5 |
| AAVrh8 | 1199 | WO2016183297A1 SEQ ID NO: 6 |
| AAV44.9 (S470N) | 1200 | WO2016183297A1 SEQ ID NO: 9 |
| rh74 VP1 | 1201 | US20160375110A1 SEQ ID NO: 1 |
| AAV-LK03 (L125I) | 1202 | WO2017015102A1 SEQ ID NO: 5 |
| AAV3B (S663V + T492V) | 1203 | WO2017015102A1 SEQ ID NO: 6 |
| Anc80 | 1204 | WO2017019994A2 SEQ ID NO: 1 |
| Anc80 | 1205 | WO2017019994A2 SEQ ID NO: 2 |
| Anc81 | 1206 | WO2017019994A2 SEQ ID NO: 3 |
| Anc81 | 1207 | WO2017019994A2 SEQ ID NO: 4 |
| Anc82 | 1208 | WO2017019994A2 SEQ ID NO: 5 |
| Anc82 | 1209 | WO2017019994A2 SEQ ID NO: 6 |
| Anc83 | 1210 | WO2017019994A2 SEQ ID NO: 7 |
| Anc83 | 1211 | WO2017019994A2 SEQ ID NO: 8 |
| Anc84 | 1212 | WO2017019994A2 SEQ ID NO: 9 |
| Anc84 | 1213 | WO2017019994A2 SEQ ID NO: 10 |
| Anc94 | 1214 | WO2017019994A2 SEQ ID NO: 11 |
| Anc94 | 1215 | WO2017019994A2 SEQ ID NO: 12 |
| Anc113 | 1216 | WO2017019994A2 SEQ ID NO: 13 |
| Anc13 | 1217 | WO2017019994A2 SEQ ID NO: 14 |
| Anc126 | 1218 | WO2017019994A2 SEQ ID NO: 15 |
| Anc126 | 1219 | WO2017019994A2 SEQ ID NO: 16 |
| Anc127 | 1220 | WO2017019994A2 SEQ ID NO: 17 |
| Anc127 | 1221 | WO2017019994A2 SEQ ID NO: 18 |
| Anc80L27 | 1222 | WO2017019994A2 SEQ ID NO: 19 |
| Anc80L59 | 1223 | WO2017019994A2 SEQ ID NO: 20 |
| Anc80L60 | 1224 | WO2017019994A2 SEQ ID NO: 21 |
| Anc80L62 | 1225 | WO2017019994A2 SEQ ID NO: 22 |
| Anc80L65 | 1226 | WO2017019994A2 SEQ ID NO: 23 |
| Anc80L33 | 1227 | WO2017019994A2 SEQ ID NO: 24 |
| Anc80L36 | 1228 | WO2017019994A2 SEQ ID NO: 25 |
| Anc80L44 | 1229 | WO2017019994A2 SEQ ID NO: 26 |
| Anc80L1 | 1230 | WO2017019994A2 SEQ ID NO: 35 |
| Anc80L1 | 1231 | WO2017019994A2 SEQ ID NO: 36 |
| AAVrh10 | 1232 | WO2017019994A2 SEQ ID NO: 41 |
| Anc110 | 1233 | WO2017019994A2 SEQ ID NO: 42 |
| Anc110 | 1234 | WO2017019994A2 SEQ ID NO: 43 |
| AAVrh32.33 | 1235 | WO2017019994A2 SEQ ID NO: 45 |
| AAVrh74 | 1236 | WO2017049031A1 SEQ ID NO: 1 |
| AAV2 | 1237 | WO2017053629A2 SEQ ID NO: 49 |
| AAV2 | 1238 | WO2017053629A2 SEQ ID NO: 50 |
| AAV2 | 1239 | WO2017053629A2 SEQ ID NO: 82 |
| Parvo-like virus | 1240 | WO2017070476A2 SEQ ID NO: 1 |
| Parvo-like virus | 1241 | WO2017070476A2 SEQ ID NO: 2 |
| Parvo-like virus | 1242 | WO2017070476A2 SEQ ID NO: 3 |
| Parvo-like virus | 1243 | WO2017070476A2 SEQ ID NO: 4 |
| Parvo-like virus | 1244 | WO2017070476A2 SEQ ID NO: 5 |
| Parvo-like virus | 1245 | WO2017070476A2 SEQ ID NO: 6 |
| AAVrh.10 | 1246 | WO2017070516A1 SEQ ID NO: 7 |
| AAVrh.10 | 1247 | WO2017070516A1 SEQ ID NO: 14 |
| AAV2tYF | 1248 | WO2017070491A1 SEQ ID NO: 1 |
| AAV-SPK | 1249 | WO2017075619A1 SEQ ID NO: 28 |
| AAV2.5 | 1250 | US20170128528A1 SEQ ID NO: 13 |
| AAV1.1 | 1251 | US20170128528A1 SEQ ID NO: 15 |
| AAV6.1 | 1252 | US20170128528A1 SEQ ID NO: 17 |
| AAV6.3.1 | 1253 | US20170128528A1 SEQ ID NO: 18 |
| AAV2i8 | 1254 | US20170128528A1 SEQ ID NO: 28 |
| AAV2i8 | 1255 | US20170128528A1 SEQ ID NO: 29 |
| ttAAV | 1256 | US20170128528A1 SEQ ID NO: 30 |
| ttAAV-S312N | 1257 | US20170128528A1 SEQ ID NO: 32 |
| ttAAV-S312N | 1258 | US20170128528A1 SEQ ID NO: 33 |
| AAV6 (Y705, Y731, and T492) | 1259 | WO2016134337A1 SEQ ID NO: 24 |
| AAV2 | 1260 | WO2016134375A1 SEQ ID NO: 9 |
| AAV2 | 1261 | WO2016134375A1 SEQ ID NO: 10 |
| AAV2 variant | 10928 | WO2018071831 SEQ ID NO: 1 |
| AAV2 variant | 10929 | WO2018071831 SEQ ID NO: 2 |
| AAV2 variant | 10930 | WO2018071831 SEQ ID NO: 3 |
| AAV2 variant | 10931 | WO2018071831 SEQ ID NO: 4 |
| AAV2 variant | 10932 | WO2018071831 SEQ ID NO: 5 |
| AAV2 variant | 10933 | WO2018071831 SEQ ID NO: 6 |
| AAV2 variant | 10934 | WO2018071831 SEQ ID NO: 7 |
| AAV2 variant | 10935 | WO2018071831 SEQ ID NO: 8 |
| AAV2 variant | 10936 | WO2018071831 SEQ ID NO: 9 |
| AAV2 variant | 10937 | WO2018071831 SEQ ID NO: 10 |
| AAV2 variant | 10938 | WO2018071831 SEQ ID NO: 11 |
| AAV2 variant | 10939 | WO2018071831 SEQ ID NO: 12 |
| AAV2 variant | 10940 | WO2018071831 SEQ ID NO: 13 |
| AAV2 variant | 10941 | WO2018071831 SEQ ID NO: 14 |
| AAV2 variant | 10942 | WO2018071831 SEQ ID NO: 15 |
| AAV2 variant | 10943 | WO2018071831 SEQ ID NO: 16 |
| AAV2 variant | 10944 | WO2018071831 SEQ ID NO: 17 |
| AAV2 variant | 10945 | WO2018071831 SEQ ID NO: 18 |
| AAV2 variant | 10946 | WO2018071831 SEQ ID NO: 19 |
| AAV2 variant | 10947 | WO2018071831 SEQ ID NO: 20 |
| AAV2 variant | 10948 | WO2018071831 SEQ ID NO: 21 |
| AAV2 variant | 10949 | WO2018071831 SEQ ID NO: 22 |
| AAV2 variant | 10950 | WO2018071831 SEQ ID NO: 23 |
| AAV2 variant | 10951 | WO2018071831 SEQ ID NO: 24 |
| AAV2 variant | 10952 | WO2018071831 SEQ ID NO: 25 |
| AAV2 variant | 10953 | WO2018071831 SEQ ID NO: 26 |
| AAV2 variant | 10954 | WO2018071831 SEQ ID NO: 27 |
| AAV2 variant | 10955 | WO2018071831 SEQ ID NO: 28 |
| AAV2 variant | 10956 | WO2018071831 SEQ ID NO: 29 |
| AAV2 variant | 10957 | WO2018071831 SEQ ID NO: 30 |
| AAV2 variant | 10958 | WO2018071831 SEQ ID NO: 31 |
| AAV2 variant | 10959 | WO2018071831 SEQ ID NO: 32 |
| AAV2 variant | 10960 | WO2018071831 SEQ ID NO: 33 |
| AAV2 variant | 10961 | WO2018071831 SEQ ID NO: 34 |
| AAV2 variant | 10962 | WO2018071831 SEQ ID NO: 35 |
| AAV2 variant | 10963 | WO2018071831 SEQ ID NO: 36 |
| AAV2 variant | 10964 | WO2018071831 SEQ ID NO: 37 |
| AAV2 variant | 10965 | WO2018071831 SEQ ID NO: 38 |
| AAV2 variant | 10966 | WO2018071831 SEQ ID NO: 39 |
| AAV2 variant | 10967 | WO2018071831 SEQ ID NO: 40 |
| AAV2 variant | 10968 | WO2018071831 SEQ ID NO: 41 |
| AAV2 variant | 10969 | WO2018071831 SEQ ID NO: 42 |
| AAV2 variant | 10970 | WO2018071831 SEQ ID NO: 43 |
| AAV2 variant | 10971 | WO2018071831 SEQ ID NO: 44 |
| AAV2 variant | 10972 | WO2018071831 SEQ ID NO: 45 |
| AAV2 variant | 10973 | WO2018071831 SEQ ID NO: 46 |
| AAV2 variant | 10974 | WO2018071831 SEQ ID NO: 47 |
| AAV2 variant | 10975 | WO2018071831 SEQ ID NO: 48 |
| AAV2 variant | 10976 | WO2018071831 SEQ ID NO: 49 |
| AAV2 variant | 10977 | WO2018071831 SEQ ID NO: 50 |
| AAV2 variant | 10978 | WO2018071831 SEQ ID NO: 51 |
| AAV2 variant | 10979 | WO2018071831 SEQ ID NO: 52 |
| AAV2 variant | 10980 | WO2018071831 SEQ ID NO: 53 |
| AAV2 variant | 10981 | WO2018071831 SEQ ID NO: 54 |
| AAV2 variant | 10982 | WO2018071831 SEQ ID NO: 55 |
| AAV2 variant | 10983 | WO2018071831 SEQ ID NO: 56 |
| AAV2 variant | 10984 | WO2018071831 SEQ ID NO: 57 |
| AAV2 variant | 10985 | WO2018071831 SEQ ID NO: 58 |
| AAV2 variant | 10986 | WO2018071831 SEQ ID NO: 59 |
| AAV2 variant | 10987 | WO2018071831 SEQ ID NO: 60 |
| AAV2 variant | 10988 | WO2018071831 SEQ ID NO: 61 |
| AAV2 variant | 10989 | WO2018071831 SEQ ID NO: 62 |
| AAV2 variant | 10990 | WO2018071831 SEQ ID NO: 63 |
| AAV2 variant | 10991 | WO2018071831 SEQ ID NO: 64 |
| AAV2 variant | 10992 | WO2018071831 SEQ ID NO: 65 |
| AAV2 variant | 10993 | WO2018071831 SEQ ID NO: 66 |
| AAV2 variant | 10994 | WO2018071831 SEQ ID NO: 67 |
| AAV2 variant | 10995 | WO2018071831 SEQ ID NO: 68 |
| AAV2 variant | 10996 | WO2018071831 SEQ ID NO: 69 |
| AAV2 variant | 10997 | WO2018071831 SEQ ID NO: 70 |
| AAV2 variant | 10998 | WO2018071831 SEQ ID NO: 71 |
| AAV2 variant | 10999 | WO2018071831 SEQ ID NO: 72 |
| AAV2 variant | 11000 | WO2018071831 SEQ ID NO: 73 |
| AAV2 variant | 11001 | WO2018071831 SEQ ID NO: 74 |
| AAV2 variant | 11002 | WO2018071831 SEQ ID NO: 75 |
| AAV2 variant | 11003 | WO2018071831 SEQ ID NO: 76 |
| AAV2 variant | 11004 | WO2018071831 SEQ ID NO: 77 |
| AAV2 variant | 11005 | WO2018071831 SEQ ID NO: 78 |
| AAV2 variant | 11006 | WO2018071831 SEQ ID NO: 79 |
| AAV2 variant | 11007 | WO2018071831 SEQ ID NO: 80 |
| AAV2 variant | 11008 | WO2018071831 SEQ ID NO: 81 |
| AAV2 variant | 11009 | WO2018071831 SEQ ID NO: 82 |
| AAV2 variant | 11010 | WO2018071831 SEQ ID NO: 83 |
| AAV2 variant | 11011 | WO2018071831 SEQ ID NO: 84 |
| AAV2 variant | 11012 | WO2018071831 SEQ ID NO: 85 |
| AAV2 variant | 11013 | WO2018071831 SEQ ID NO: 86 |
| AAV2 variant | 11014 | WO2018071831 SEQ ID NO: 87 |
| AAV2 variant | 11015 | WO2018071831 SEQ ID NO: 88 |
| AAV2 variant | 11016 | WO2018071831 SEQ ID NO: 89 |
| AAV2 variant | 11017 | WO2018071831 SEQ ID NO: 90 |
| AAV2 variant | 11018 | WO2018071831 SEQ ID NO: 91 |
| AAV2 variant | 11019 | WO2018071831 SEQ ID NO: 92 |
| AAV2 variant | 11020 | WO2018071831 SEQ ID NO: 93 |
| AAV2 variant | 11021 | WO2018071831 SEQ ID NO: 94 |
| AAV2 variant | 11022 | WO2018071831 SEQ ID NO: 95 |
| AAV2 variant | 11023 | WO2018071831 SEQ ID NO: 96 |
| AAV2 variant | 11024 | WO2018071831 SEQ ID NO: 97 |
| AAV2 variant | 11025 | WO2018071831 SEQ ID NO: 98 |
| AAV2 variant | 11026 | WO2018071831 SEQ ID NO: 99 |
| AAV2 variant | 11027 | WO2018071831 SEQ ID NO: 100 |
| AAV2 variant | 11028 | WO2018071831 SEQ ID NO: 101 |
| AAV2 variant | 11029 | WO2018071831 SEQ ID NO: 102 |
| AAV2 variant | 11030 | WO2018071831 SEQ ID NO: 103 |
| AAV2 variant | 11031 | WO2018071831 SEQ ID NO: 104 |
| AAV2 variant | 11032 | WO2018071831 SEQ ID NO: 105 |
| AAV2 variant | 11033 | WO2018071831 SEQ ID NO: 106 |
| AAV2 variant | 11034 | WO2018071831 SEQ ID NO: 107 |
| AAV2 variant | 11035 | WO2018071831 SEQ ID NO: 108 |
| AAV2 variant | 11036 | WO2018071831 SEQ ID NO: 109 |
| AAV2 variant | 11037 | WO2018071831 SEQ ID NO: 110 |
| AAV2 variant | 11038 | WO2018071831 SEQ ID NO: 111 |
| AAV2 variant | 11039 | WO2018071831 SEQ ID NO: 112 |
| AAV2 variant | 11040 | WO2018071831 SEQ ID NO: 113 |
| AAV2 variant | 11041 | WO2018071831 SEQ ID NO: 114 |
| AAV2 variant | 11042 | WO2018071831 SEQ ID NO: 115 |
| AAV2 variant | 11043 | WO2018071831 SEQ ID NO: 116 |
| AAV2 variant | 11044 | WO2018071831 SEQ ID NO: 117 |
| AAV2 variant | 11045 | WO2018071831 SEQ ID NO: 118 |
| AAV2 variant | 11046 | WO2018071831 SEQ ID NO: 119 |
| AAV2 variant | 11047 | WO2018071831 SEQ ID NO: 120 |
| AAV2 variant | 11048 | WO2018071831 SEQ ID NO: 121 |
| AAV2 variant | 11049 | WO2018071831 SEQ ID NO: 122 |
| AAV2 variant | 11050 | WO2018071831 SEQ ID NO: 123 |
| AAV2 variant | 11051 | WO2018071831 SEQ ID NO: 124 |
| AAV2 variant | 11052 | WO2018071831 SEQ ID NO: 125 |
| AAV2 variant | 11053 | WO2018071831 SEQ ID NO: 126 |
| AAV2 variant | 11054 | WO2018071831 SEQ ID NO: 127 |
| AAV2 variant | 11055 | WO2018071831 SEQ ID NO: 128 |
| AAV2 variant | 11056 | WO2018071831 SEQ ID NO: 129 |
| AAV2 variant | 11057 | WO2018071831 SEQ ID NO: 130 |
| AAV2 variant | 11058 | WO2018071831 SEQ ID NO: 131 |
| AAV2 variant | 11059 | WO2018071831 SEQ ID NO: 132 |
| AAV2 variant | 11060 | WO2018071831 SEQ ID NO: 133 |
| AAV2 variant | 11061 | WO2018071831 SEQ ID NO: 134 |
| AAV2 variant | 11062 | WO2018071831 SEQ ID NO: 135 |
| AAV2 variant | 11063 | WO2018071831 SEQ ID NO: 136 |
| AAV2 variant | 11064 | WO2018071831 SEQ ID NO: 137 |
| AAV2 variant | 11065 | WO2018071831 SEQ ID NO: 138 |
| AAV2 variant | 11066 | WO2018071831 SEQ ID NO: 139 |
| AAV2 variant | 11067 | WO2018071831 SEQ ID NO: 140 |
| AAV2 variant | 11068 | WO2018071831 SEQ ID NO: 141 |
| AAV2 variant | 11069 | WO2018071831 SEQ ID NO: 142 |
| AAV2 variant | 11070 | WO2018071831 SEQ ID NO: 143 |
| AAV2 variant | 11071 | WO2018071831 SEQ ID NO: 144 |
| AAV2 variant | 11072 | WO2018071831 SEQ ID NO: 145 |
| AAV2 variant | 11073 | WO2018071831 SEQ ID NO: 146 |
| AAV2 variant | 11074 | WO2018071831 SEQ ID NO: 147 |
| AAV2 variant | 11075 | WO2018071831 SEQ ID NO: 148 |
| AAV2 variant | 11076 | WO2018071831 SEQ ID NO: 149 |
| AAV2 variant | 11077 | WO2018071831 SEQ ID NO: 150 |
| AAV2 variant | 11078 | WO2018071831 SEQ ID NO: 151 |
| AAV2 variant | 11079 | WO2018071831 SEQ ID NO: 152 |
| AAV2 variant | 11080 | WO2018071831 SEQ ID NO: 153 |
| AAV2 variant | 11081 | WO2018071831 SEQ ID NO: 154 |
| AAV2 variant | 11082 | WO2018071831 SEQ ID NO: 155 |
| AAV2 variant | 11083 | WO2018071831 SEQ ID NO: 156 |
| AAV2 variant | 11084 | WO2018071831 SEQ ID NO: 157 |
| AAV2 variant | 11085 | WO2018071831 SEQ ID NO: 158 |
| AAV2 variant | 11086 | WO2018071831 SEQ ID NO: 159 |
| AAV2 variant | 11087 | WO2018071831 SEQ ID NO: 160 |
| AAV2 variant | 11088 | WO2018071831 SEQ ID NO: 161 |
| AAV2 variant | 11089 | WO2018071831 SEQ ID NO: 162 |
| AAV2 variant | 11090 | WO2018071831 SEQ ID NO: 163 |
| AAV2 variant | 11091 | WO2018071831 SEQ ID NO: 164 |
| AAV2 variant | 11092 | WO2018071831 SEQ ID NO: 165 |
| AAV2 variant | 11093 | WO2018071831 SEQ ID NO: 166 |
| AAV2 variant | 11094 | WO2018071831 SEQ ID NO: 167 |
| AAV2 variant | 11095 | WO2018071831 SEQ ID NO: 168 |
| AAV2 variant | 11096 | WO2018071831 SEQ ID NO: 169 |
| AAV2 variant | 11097 | WO2018071831 SEQ ID NO: 170 |
| AAV2 variant | 11098 | WO2018071831 SEQ ID NO: 171 |
| AAV2 variant | 11099 | WO2018071831 SEQ ID NO: 172 |
| AAV2 variant | 11100 | WO2018071831 SEQ ID NO: 173 |
| AAV2 variant | 11101 | WO2018071831 SEQ ID NO: 174 |
| AAV2 variant | 11102 | WO2018071831 SEQ ID NO: 175 |
| AAV2 variant | 11103 | WO2018071831 SEQ ID NO: 176 |
| AAV2 variant | 11104 | WO2018071831 SEQ ID NO: 177 |
| AAV2 variant | 11105 | WO2018071831 SEQ ID NO: 178 |
| AAV2 variant | 11106 | WO2018071831 SEQ ID NO: 179 |
| AAV2 variant | 11107 | WO2018071831 SEQ ID NO: 180 |
| AAV2 variant | 11108 | WO2018071831 SEQ ID NO: 181 |
| AAV2 variant | 11109 | WO2018071831 SEQ ID NO: 182 |
| AAV2 variant | 11110 | WO2018071831 SEQ ID NO: 183 |
| AAV2 variant | 11111 | WO2018071831 SEQ ID NO: 184 |
| AAV2 variant | 11112 | WO2018071831 SEQ ID NO: 185 |
| AAV2 variant | 11113 | WO2018071831 SEQ ID NO: 186 |
| AAV2 variant | 11114 | WO2018071831 SEQ ID NO: 187 |
| AAV2 variant | 11115 | WO2018071831 SEQ ID NO: 188 |
| AAV2 variant | 11116 | WO2018071831 SEQ ID NO: 189 |
| AAV2 variant | 11117 | WO2018071831 SEQ ID NO: 190 |
| AAV2 variant | 11118 | WO2018071831 SEQ ID NO: 191 |
| AAV2 variant | 11119 | WO2018071831 SEQ ID NO: 192 |
| AAV2 variant | 11120 | WO2018071831 SEQ ID NO: 193 |
| AAV2 variant | 11121 | WO2018071831 SEQ ID NO: 194 |
| AAV2 variant | 11122 | WO2018071831 SEQ ID NO: 195 |
| AAV2 variant | 11123 | WO2018071831 SEQ ID NO: 196 |
| AAV2 variant | 11124 | WO2018071831 SEQ ID NO: 197 |
| AAV2 variant | 11125 | WO2018071831 SEQ ID NO: 198 |
| AAV2 variant | 11126 | WO2018071831 SEQ ID NO: 199 |
| AAV2 variant | 11127 | WO2018071831 SEQ ID NO: 200 |
| AAV2 variant | 11128 | WO2018071831 SEQ ID NO: 201 |
| AAV2 variant | 11129 | WO2018071831 SEQ ID NO: 202 |
| AAV2 variant | 11130 | WO2018071831 SEQ ID NO: 203 |
| AAV2 variant | 11131 | WO2018071831 SEQ ID NO: 204 |
| AAV2 variant | 11132 | WO2018071831 SEQ ID NO: 205 |
| AAV2 variant | 11133 | WO2018071831 SEQ ID NO: 206 |
| AAV2 variant | 11134 | WO2018071831 SEQ ID NO: 207 |
| AAV2 variant | 11135 | WO2018071831 SEQ ID NO: 208 |
| AAV2 variant | 11136 | WO2018071831 SEQ ID NO: 209 |
| AAV2 variant | 11137 | WO2018071831 SEQ ID NO: 210 |
| AAV2 variant | 11138 | WO2018071831 SEQ ID NO: 211 |
| AAV2 variant | 11139 | WO2018071831 SEQ ID NO: 212 |
| AAV2 variant | 11140 | WO2018071831 SEQ ID NO: 213 |
| AAV2 variant | 11141 | WO2018071831 SEQ ID NO: 214 |
| AAV2 variant | 11142 | WO2018071831 SEQ ID NO: 215 |
| AAV2 variant | 11143 | WO2018071831 SEQ ID NO: 216 |
| AAV2 variant | 11144 | WO2018071831 SEQ ID NO: 217 |
| AAV2 variant | 11145 | WO2018071831 SEQ ID NO: 218 |
| AAV2 variant | 11146 | WO2018071831 SEQ ID NO: 219 |
| AAV2 variant | 11147 | WO2018071831 SEQ ID NO: 220 |
| AAV2 variant | 11148 | WO2018071831 SEQ ID NO: 221 |
| AAV2 variant | 11149 | WO2018071831 SEQ ID NO: 222 |
| AAV2 variant | 11150 | WO2018071831 SEQ ID NO: 223 |
| AAV2 variant | 11151 | WO2018071831 SEQ ID NO: 224 |
| AAV2 variant | 11152 | WO2018071831 SEQ ID NO: 225 |
| AAV2 variant | 11153 | WO2018071831 SEQ ID NO: 226 |
| AAV2 variant | 11154 | WO2018071831 SEQ ID NO: 227 |
| AAV2 variant | 11155 | WO2018071831 SEQ ID NO: 228 |
| AAV2 variant | 11156 | WO2018071831 SEQ ID NO: 229 |
| AAV2 variant | 11157 | WO2018071831 SEQ ID NO: 230 |
| AAV2 variant | 11158 | WO2018071831 SEQ ID NO: 231 |
| AAV2 variant | 11159 | WO2018071831 SEQ ID NO: 232 |
| AAV2 variant | 11160 | WO2018071831 SEQ ID NO: 233 |
| AAV2 variant | 11161 | WO2018071831 SEQ ID NO: 234 |
| AAV2 variant | 11162 | WO2018071831 SEQ ID NO: 235 |
| AAV2 variant | 11163 | WO2018071831 SEQ ID NO: 236 |
| AAV2 variant | 11164 | WO2018071831 SEQ ID NO: 237 |
| AAV2 variant | 11165 | WO2018071831 SEQ ID NO: 238 |
| AAV2 variant | 11166 | WO2018071831 SEQ ID NO: 239 |
| AAV2 variant | 11167 | WO2018071831 SEQ ID NO: 240 |
| AAV2 variant | 11168 | WO2018071831 SEQ ID NO: 241 |
| AAV2 variant | 11169 | WO2018071831 SEQ ID NO: 242 |
| AAV2 variant | 11170 | WO2018071831 SEQ ID NO: 243 |
| AAV2 variant | 11171 | WO2018071831 SEQ ID NO: 244 |
| AAV2 variant | 11172 | WO2018071831 SEQ ID NO: 245 |
| AAV2 variant | 11173 | WO2018071831 SEQ ID NO: 246 |
| AAV2 variant | 11174 | WO2018071831 SEQ ID NO: 247 |
| AAV2 variant | 11175 | WO2018071831 SEQ ID NO: 248 |
| AAV2 variant | 11176 | WO2018071831 SEQ ID NO: 249 |
| AAV2 variant | 11177 | WO2018071831 SEQ ID NO: 250 |
| AAV2 variant | 11178 | WO2018071831 SEQ ID NO: 251 |
| AAV2 variant | 11179 | WO2018071831 SEQ ID NO: 252 |
| AAV2 variant | 11180 | WO2018071831 SEQ ID NO: 253 |
| AAV2 variant | 11181 | WO2018071831 SEQ ID NO: 254 |
| AAV2 variant | 11182 | WO2018071831 SEQ ID NO: 255 |
| AAV2 variant | 11183 | WO2018071831 SEQ ID NO: 256 |
| AAV2 variant | 11184 | WO2018071831 SEQ ID NO: 257 |
| AAV2 variant | 11185 | WO2018071831 SEQ ID NO: 258 |
| AAV2 variant | 11186 | WO2018071831 SEQ ID NO: 259 |
| AAV2 variant | 11187 | WO2018071831 SEQ ID NO: 260 |
| AAV2 variant | 11188 | WO2018071831 SEQ ID NO: 261 |
| AAV2 variant | 11189 | WO2018071831 SEQ ID NO: 262 |
| AAV2 variant | 11190 | WO2018071831 SEQ ID NO: 263 |
| AAV2 variant | 11191 | WO2018071831 SEQ ID NO: 264 |
| AAV2 variant | 11192 | WO2018071831 SEQ ID NO: 265 |
| AAV2 variant | 11193 | WO2018071831 SEQ ID NO: 266 |
| AAV2 variant | 11194 | WO2018071831 SEQ ID NO: 267 |
| AAV2 variant | 11195 | WO2018071831 SEQ ID NO: 268 |
| AAV2 variant | 11196 | WO2018071831 SEQ ID NO: 269 |
| AAV2 variant | 11197 | WO2018071831 SEQ ID NO: 270 |
| AAV2 variant | 11198 | WO2018071831 SEQ ID NO: 271 |
| AAV2 variant | 11199 | WO2018071831 SEQ ID NO: 272 |
| AAV2 variant | 11200 | WO2018071831 SEQ ID NO: 273 |
| AAV2 variant | 11201 | WO2018071831 SEQ ID NO: 274 |
| AAV2 variant | 11202 | WO2018071831 SEQ ID NO: 275 |
| AAV2 variant | 11203 | WO2018071831 SEQ ID NO: 276 |
| AAV2 variant | 11204 | WO2018071831 SEQ ID NO: 277 |
| AAV2 variant | 11205 | WO2018071831 SEQ ID NO: 278 |
| AAV2 variant | 11206 | WO2018071831 SEQ ID NO: 279 |
| AAV2 variant | 11207 | WO2018071831 SEQ ID NO: 280 |
| AAV2 variant | 11208 | WO2018071831 SEQ ID NO: 281 |
| AAV2 variant | 11209 | WO2018071831 SEQ ID NO: 282 |
| AAV2 variant | 11210 | WO2018071831 SEQ ID NO: 283 |
| AAV2 variant | 11211 | WO2018071831 SEQ ID NO: 284 |
| AAV2 variant | 11212 | WO2018071831 SEQ ID NO: 285 |
| AAV2 variant | 11213 | WO2018071831 SEQ ID NO: 286 |
| AAV2 variant | 11214 | WO2018071831 SEQ ID NO: 287 |
| AAV2 variant | 11215 | WO2018071831 SEQ ID NO: 288 |
| AAV2 variant | 11216 | WO2018071831 SEQ ID NO: 289 |
| AAV2 variant | 11217 | WO2018071831 SEQ ID NO: 290 |
| AAV2 variant | 11218 | WO2018071831 SEQ ID NO: 291 |
| AAV2 variant | 11219 | WO2018071831 SEQ ID NO: 292 |
| AAV2 variant | 11220 | WO2018071831 SEQ ID NO: 293 |
| AAV2 variant | 11221 | WO2018071831 SEQ ID NO: 294 |
| AAV2 variant | 11222 | WO2018071831 SEQ ID NO: 295 |
| AAV2 variant | 11223 | WO2018071831 SEQ ID NO: 296 |
| AAV2 variant | 11224 | WO2018071831 SEQ ID NO: 297 |
| AAV2 variant | 11225 | WO2018071831 SEQ ID NO: 298 |
| AAV2 variant | 11226 | WO2018071831 SEQ ID NO: 299 |
| AAV2 variant | 11227 | WO2018071831 SEQ ID NO: 300 |
| AAV2 variant | 11228 | WO2018071831 SEQ ID NO: 301 |
| AAV2 variant | 11229 | WO2018071831 SEQ ID NO: 302 |
| AAV2 variant | 11230 | WO2018071831 SEQ ID NO: 303 |
| AAV2 variant | 11231 | WO2018071831 SEQ ID NO: 304 |
| AAV2 variant | 11232 | WO2018071831 SEQ ID NO: 305 |
| AAV2 variant | 11233 | WO2018071831 SEQ ID NO: 306 |
| AAV2 variant | 11234 | WO2018071831 SEQ ID NO: 307 |
| AAV2 variant | 11235 | WO2018071831 SEQ ID NO: 308 |
| AAV2 variant | 11236 | WO2018071831 SEQ ID NO: 309 |
| AAV2 variant | 11237 | WO2018071831 SEQ ID NO: 310 |
| AAV2 variant | 11238 | WO2018071831 SEQ ID NO: 311 |
| AAV2 variant | 11239 | WO2018071831 SEQ ID NO: 312 |
| AAV2 variant | 11240 | WO2018071831 SEQ ID NO: 313 |
| AAV2 variant | 11241 | WO2018071831 SEQ ID NO: 314 |
| AAV2 variant | 11242 | WO2018071831 SEQ ID NO: 315 |
| AAV2 variant | 11243 | WO2018071831 SEQ ID NO: 316 |
| AAV2 variant | 11244 | WO2018071831 SEQ ID NO: 317 |
| AAV2 variant | 11245 | WO2018071831 SEQ ID NO: 318 |
| AAV2 variant | 11246 | WO2018071831 SEQ ID NO: 319 |
| AAV2 variant | 11247 | WO2018071831 SEQ ID NO: 320 |
| AAV2 variant | 11248 | WO2018071831 SEQ ID NO: 321 |
| AAV2 variant | 11249 | WO2018071831 SEQ ID NO: 322 |
| AAV2 variant | 11250 | WO2018071831 SEQ ID NO: 323 |
| AAV2 variant | 11251 | WO2018071831 SEQ ID NO: 324 |
| AAV2 variant | 11252 | WO2018071831 SEQ ID NO: 325 |
| AAV2 variant | 11253 | WO2018071831 SEQ ID NO: 326 |
| AAV2 variant | 11254 | WO2018071831 SEQ ID NO: 327 |
| AAV2 variant | 11255 | WO2018071831 SEQ ID NO: 328 |
| AAV2 variant | 11256 | WO2018071831 SEQ ID NO: 329 |
| AAV2 variant | 11257 | WO2018071831 SEQ ID NO: 330 |
| AAV2 variant | 11258 | WO2018071831 SEQ ID NO: 331 |
| AAV2 variant | 11259 | WO2018071831 SEQ ID NO: 332 |
| AAV2 variant | 11260 | WO2018071831 SEQ ID NO: 333 |
| AAV2 variant | 11261 | WO2018071831 SEQ ID NO: 334 |
| AAV2 variant | 11262 | WO2018071831 SEQ ID NO: 335 |
| AAV2 variant | 11263 | WO2018071831 SEQ ID NO: 336 |
| AAV2 variant | 11264 | WO2018071831 SEQ ID NO: 337 |
| AAV2 variant | 11265 | WO2018071831 SEQ ID NO: 338 |
| AAV2 variant | 11266 | WO2018071831 SEQ ID NO: 339 |
| AAV2 variant | 11267 | WO2018071831 SEQ ID NO: 340 |
| AAV2 variant | 11268 | WO2018071831 SEQ ID NO: 341 |
| AAV2 variant | 11269 | WO2018071831 SEQ ID NO: 342 |
| AAV2 variant | 11270 | WO2018071831 SEQ ID NO: 343 |
| AAV2 variant | 11271 | WO2018071831 SEQ ID NO: 344 |
| AAV2 variant | 11272 | WO2018071831 SEQ ID NO: 345 |
| AAV2 variant | 11273 | WO2018071831 SEQ ID NO: 346 |
| AAV2 variant | 11274 | WO2018071831 SEQ ID NO: 347 |
| AAV2 variant | 11275 | WO2018071831 SEQ ID NO: 348 |
| AAV2 variant | 11276 | WO2018071831 SEQ ID NO: 349 |
| AAV2 variant | 11277 | WO2018071831 SEQ ID NO: 350 |
| AAV2 variant | 11278 | WO2018071831 SEQ ID NO: 351 |
| AAV2 variant | 11279 | WO2018071831 SEQ ID NO: 352 |
| AAV2 variant | 11280 | WO2018071831 SEQ ID NO: 353 |
| AAV2 variant | 11281 | WO2018071831 SEQ ID NO: 354 |
| AAV2 variant | 11282 | WO2018071831 SEQ ID NO: 355 |
| AAV2 variant | 11283 | WO2018071831 SEQ ID NO: 356 |
| AAV2 variant | 11284 | WO2018071831 SEQ ID NO: 357 |
| AAV2 variant | 11285 | WO2018071831 SEQ ID NO: 358 |
| AAV2 variant | 11286 | WO2018071831 SEQ ID NO: 359 |
| AAV2 variant | 11287 | WO2018071831 SEQ ID NO: 360 |
| AAV2 variant | 11288 | WO2018071831 SEQ ID NO: 361 |
| AAV2 variant | 11289 | WO2018071831 SEQ ID NO: 362 |
| AAV2 variant | 11290 | WO2018071831 SEQ ID NO: 363 |
| AAV2 variant | 11291 | WO2018071831 SEQ ID NO: 364 |
| AAV2 variant | 11292 | WO2018071831 SEQ ID NO: 365 |
| AAV2 variant | 11293 | WO2018071831 SEQ ID NO: 366 |
| AAV2 variant | 11294 | WO2018071831 SEQ ID NO: 367 |
| AAV2 variant | 11295 | WO2018071831 SEQ ID NO: 368 |
| AAV2 variant | 11296 | WO2018071831 SEQ ID NO: 369 |
| AAV2 variant | 11297 | WO2018071831 SEQ ID NO: 370 |
| AAV2 variant | 11298 | WO2018071831 SEQ ID NO: 371 |
| AAV2 variant | 11299 | WO2018071831 SEQ ID NO: 372 |
| AAV2 variant | 11300 | WO2018071831 SEQ ID NO: 373 |
| AAV2 variant | 11301 | WO2018071831 SEQ ID NO: 374 |
| AAV2 variant | 11302 | WO2018071831 SEQ ID NO: 375 |
| AAV2 variant | 11303 | WO2018071831 SEQ ID NO: 376 |
| AAV2 variant | 11304 | WO2018071831 SEQ ID NO: 377 |
| AAV2 variant | 11305 | WO2018071831 SEQ ID NO: 378 |
| AAV2 variant | 11306 | WO2018071831 SEQ ID NO: 379 |
| AAV2 variant | 11307 | WO2018071831 SEQ ID NO: 380 |
| AAV2 variant | 11308 | WO2018071831 SEQ ID NO: 381 |
| AAV2 variant | 11309 | WO2018071831 SEQ ID NO: 382 |
| AAV2 variant | 11310 | WO2018071831 SEQ ID NO: 383 |
| AAV2 variant | 11311 | WO2018071831 SEQ ID NO: 384 |
| AAV2 variant | 11312 | WO2018071831 SEQ ID NO: 385 |
| AAV2 variant | 11313 | WO2018071831 SEQ ID NO: 386 |
| AAV2 variant | 11314 | WO2018071831 SEQ ID NO: 387 |
| AAV2 variant | 11315 | WO2018071831 SEQ ID NO: 388 |
| AAV2 variant | 11316 | WO2018071831 SEQ ID NO: 389 |
| AAV2 variant | 11317 | WO2018071831 SEQ ID NO: 390 |
| AAV2 variant | 11318 | WO2018071831 SEQ ID NO: 391 |
| AAV2 variant | 11319 | WO2018071831 SEQ ID NO: 392 |
| AAV2 variant | 11320 | WO2018071831 SEQ ID NO: 393 |
| AAV2 variant | 11321 | WO2018071831 SEQ ID NO: 394 |
| AAV2 variant | 11322 | WO2018071831 SEQ ID NO: 395 |
| AAV2 variant | 11323 | WO2018071831 SEQ ID NO: 396 |
| AAV2 variant | 11324 | WO2018071831 SEQ ID NO: 397 |
| AAV2 variant | 11325 | WO2018071831 SEQ ID NO: 398 |
| AAV2 variant | 11326 | WO2018071831 SEQ ID NO: 399 |
| AAV2 variant | 11327 | WO2018071831 SEQ ID NO: 400 |
| AAV2 variant | 11328 | WO2018071831 SEQ ID NO: 401 |
| AAV2 variant | 11329 | WO2018071831 SEQ ID NO: 402 |
| AAV2 variant | 11330 | WO2018071831 SEQ ID NO: 403 |
| AAV2 variant | 11331 | WO2018071831 SEQ ID NO: 404 |
| AAV2 variant | 11332 | WO2018071831 SEQ ID NO: 405 |
| AAV2 variant | 11333 | WO2018071831 SEQ ID NO: 406 |
| AAV2 variant | 11334 | WO2018071831 SEQ ID NO: 407 |
| AAV2 variant | 11335 | WO2018071831 SEQ ID NO: 408 |
| AAV2 variant | 11336 | WO2018071831 SEQ ID NO: 409 |
| AAV2/3 variant | 11337 | WO2018071831 SEQ ID NO: 435 |
| AAV2/3 variant | 11338 | WO2018071831 SEQ ID NO: 436 |
| AAV2/3 variant | 11339 | WO2018071831 SEQ ID NO: 437 |
| AAV2/3 variant | 11340 | WO2018071831 SEQ ID NO: 438 |
| AAV2/3 variant | 11341 | WO2018071831 SEQ ID NO: 439 |
| AAV2/3 variant | 11342 | WO2018071831 SEQ ID NO: 440 |
| AAV2/3 variant | 11343 | WO2018071831 SEQ ID NO: 441 |
| AAV2/3 variant | 11344 | WO2018071831 SEQ ID NO: 442 |
| AAV2/3 variant | 11345 | WO2018071831 SEQ ID NO: 443 |
| AAV2/3 variant | 11346 | WO2018071831 SEQ ID NO: 444 |
| AAV2/3 variant | 11347 | WO2018071831 SEQ ID NO: 445 |
| AAV2/3 variant | 11348 | WO2018071831 SEQ ID NO: 446 |
| AAV2/3 variant | 11349 | WO2018071831 SEQ ID NO: 447 |
| AAV2/3 variant | 11350 | WO2018071831 SEQ ID NO: 448 |
| AAV2/3 variant | 11351 | WO2018071831 SEQ ID NO: 449 |
| AAV2/3 variant | 11352 | WO2018071831 SEQ ID NO: 450 |
| AAV2/3 variant | 11353 | WO2018071831 SEQ ID NO: 451 |
| AAV2/3 variant | 11354 | WO2018071831 SEQ ID NO: 452 |
| AAV2/3 variant | 11355 | WO2018071831 SEQ ID NO: 453 |
| AAV2/3 variant | 11356 | WO2018071831 SEQ ID NO: 454 |
| AAV2/3 variant | 11357 | WO2018071831 SEQ ID NO: 455 |
| AAV2/3 variant | 11358 | WO2018071831 SEQ ID NO: 456 |
| AAV2/3 variant | 11359 | WO2018071831 SEQ ID NO: 457 |
| AAV2/3 variant | 11360 | WO2018071831 SEQ ID NO: 458 |
| AAV2/3 variant | 11361 | WO2018071831 SEQ ID NO: 459 |
| AAV2/3 variant | 11362 | WO2018071831 SEQ ID NO: 460 |
| AAV2/3 variant | 11363 | WO2018071831 SEQ ID NO: 461 |
| AAV2/3 variant | 11364 | WO2018071831 SEQ ID NO: 462 |
| AAV2/3 variant | 11365 | WO2018071831 SEQ ID NO: 463 |
| AAV2/3 variant | 11366 | WO2018071831 SEQ ID NO: 464 |
| AAV2/3 variant | 11367 | WO2018071831 SEQ ID NO: 465 |
| AAV2/3 variant | 11368 | WO2018071831 SEQ ID NO: 466 |
| AAV2/3 variant | 11369 | WO2018071831 SEQ ID NO: 467 |
| AAV2/3 variant | 11370 | WO2018071831 SEQ ID NO: 468 |
| AAV2/3 variant | 11371 | WO2018071831 SEQ ID NO: 469 |
| AAV2/3 variant | 11372 | WO2018071831 SEQ ID NO: 470 |
| AAV2/3 variant | 11373 | WO2018071831 SEQ ID NO: 471 |
| AAV2/3 variant | 11374 | WO2018071831 SEQ ID NO: 472 |
| AAV2/3 variant | 11375 | WO2018071831 SEQ ID NO: 473 |
| AAV2/3 variant | 11376 | WO2018071831 SEQ ID NO: 474 |
| AAV2/3 variant | 11377 | WO2018071831 SEQ ID NO: 475 |
| AAV2/3 variant | 11378 | WO2018071831 SEQ ID NO: 476 |
| AAV2/3 variant | 11379 | WO2018071831 SEQ ID NO: 477 |
| AAV2/3 variant | 11380 | WO2018071831 SEQ ID NO: 478 |
| AAV2/3 variant | 11381 | WO2018071831 SEQ ID NO: 479 |
| AAV2/3 variant | 11382 | WO2018071831 SEQ ID NO: 480 |
| AAV2/3 variant | 11383 | WO2018071831 SEQ ID NO: 481 |
| AAV2/3 variant | 11384 | WO2018071831 SEQ ID NO: 482 |
| AAV2/3 variant | 11385 | WO2018071831 SEQ ID NO: 483 |
| AAV2/3 variant | 11386 | WO2018071831 SEQ ID NO: 484 |
| AAV2/3 variant | 11387 | WO2018071831 SEQ ID NO: 485 |
| AAV2/3 variant | 11388 | WO2018071831 SEQ ID NO: 486 |
| AAV2/3 variant | 11389 | WO2018071831 SEQ ID NO: 487 |
| AAV2/3 variant | 11390 | WO2018071831 SEQ ID NO: 488 |
| AAV2/3 variant | 11391 | WO2018071831 SEQ ID NO: 489 |
| AAV2/3 variant | 11392 | WO2018071831 SEQ ID NO: 490 |
| AAV2/3 variant | 11393 | WO2018071831 SEQ ID NO: 491 |
| AAV2/3 variant | 11394 | WO2018071831 SEQ ID NO: 492 |
| AAV2/3 variant | 11395 | WO2018071831 SEQ ID NO: 493 |
| AAV2/3 variant | 11396 | WO2018071831 SEQ ID NO: 494 |
| AAV2/3 variant | 11397 | WO2018071831 SEQ ID NO: 495 |
| AAV2/3 variant | 11398 | WO2018071831 SEQ ID NO: 496 |
| AAV2/3 variant | 11399 | WO2018071831 SEQ ID NO: 497 |
| AAV2/3 variant | 11400 | WO2018071831 SEQ ID NO: 498 |
| AAV2/3 variant | 11401 | WO2018071831 SEQ ID NO: 499 |
| AAV2/3 variant | 11402 | WO2018071831 SEQ ID NO: 500 |
| AAV2/3 variant | 11403 | WO2018071831 SEQ ID NO: 501 |
| AAV2/3 variant | 11404 | WO2018071831 SEQ ID NO: 502 |
| AAV2/3 variant | 11405 | WO2018071831 SEQ ID NO: 503 |
| AAV2/3 variant | 11406 | WO2018071831 SEQ ID NO: 504 |
| AAV2/3 variant | 11407 | WO2018071831 SEQ ID NO: 505 |
| AAV2/3 variant | 11408 | WO2018071831 SEQ ID NO: 506 |
| AAV2/3 variant | 11409 | WO2018071831 SEQ ID NO: 507 |
| AAV2/3 variant | 11410 | WO2018071831 SEQ ID NO: 508 |
| AAV2/3 variant | 11411 | WO2018071831 SEQ ID NO: 509 |
| AAV2/3 variant | 11412 | WO2018071831 SEQ ID NO: 510 |
| AAV2/3 variant | 11413 | WO2018071831 SEQ ID NO: 511 |
| AAV2/3 variant | 11414 | WO2018071831 SEQ ID NO: 512 |
| AAV2/3 variant | 11415 | WO2018071831 SEQ ID NO: 513 |
| AAV2/3 variant | 11416 | WO2018071831 SEQ ID NO: 514 |
| AAV2/3 variant | 11417 | WO2018071831 SEQ ID NO: 515 |
| AAV2/3 variant | 11418 | WO2018071831 SEQ ID NO: 516 |
| AAV2/3 variant | 11419 | WO2018071831 SEQ ID NO: 517 |
| AAV2/3 variant | 11420 | WO2018071831 SEQ ID NO: 518 |
| AAV2/3 variant | 11421 | WO2018071831 SEQ ID NO: 519 |
| AAV2/3 variant | 11422 | WO2018071831 SEQ ID NO: 520 |
| AAV2/3 variant | 11423 | WO2018071831 SEQ ID NO: 521 |
| AAV2/3 variant | 11424 | WO2018071831 SEQ ID NO: 522 |
| AAV2/3 variant | 11425 | WO2018071831 SEQ ID NO: 523 |
| AAV2/3 variant | 11426 | WO2018071831 SEQ ID NO: 524 |
| AAV2/3 variant | 11427 | WO2018071831 SEQ ID NO: 525 |
| AAV2/3 variant | 11428 | WO2018071831 SEQ ID NO: 526 |
| AAV2/3 variant | 11429 | WO2018071831 SEQ ID NO: 527 |
| AAV2/3 variant | 11430 | WO2018071831 SEQ ID NO: 528 |
| AAV2/3 variant | 11431 | WO2018071831 SEQ ID NO: 529 |
| AAV2/3 variant | 11432 | WO2018071831 SEQ ID NO: 530 |
| AAV2/3 variant | 11433 | WO2018071831 SEQ ID NO: 531 |
| AAV2/3 variant | 11434 | WO2018071831 SEQ ID NO: 532 |
| AAV2/3 variant | 11435 | WO2018071831 SEQ ID NO: 533 |
| AAV2/3 variant | 11436 | WO2018071831 SEQ ID NO: 534 |
| AAV2/3 variant | 11437 | WO2018071831 SEQ ID NO: 535 |
| AAV2/3 variant | 11438 | WO2018071831 SEQ ID NO: 536 |
| AAV2/3 variant | 11439 | WO2018071831 SEQ ID NO: 537 |
| AAV2/3 variant | 11440 | WO2018071831 SEQ ID NO: 538 |
| AAV2/3 variant | 11441 | WO2018071831 SEQ ID NO: 539 |
| AAV2/3 variant | 11442 | WO2018071831 SEQ ID NO: 540 |
| AAV2/3 variant | 11443 | WO2018071831 SEQ ID NO: 541 |
| AAV2/3 variant | 11444 | WO2018071831 SEQ ID NO: 542 |
| AAV2/3 variant | 11445 | WO2018071831 SEQ ID NO: 543 |
| AAV2/3 variant | 11446 | WO2018071831 SEQ ID NO: 544 |
| AAV2/3 variant | 11447 | WO2018071831 SEQ ID NO: 545 |
| AAV2/3 variant | 11448 | WO2018071831 SEQ ID NO: 546 |
| AAV2/3 variant | 11449 | WO2018071831 SEQ ID NO: 547 |
| AAV2/3 variant | 11450 | WO2018071831 SEQ ID NO: 548 |
| AAV2/3 variant | 11451 | WO2018071831 SEQ ID NO: 549 |
| AAV2/3 variant | 11452 | WO2018071831 SEQ ID NO: 550 |
| AAV2/3 variant | 11453 | WO2018071831 SEQ ID NO: 551 |
| AAV2/3 variant | 11454 | WO2018071831 SEQ ID NO: 552 |
| AAV2/3 variant | 11455 | WO2018071831 SEQ ID NO: 553 |
| AAV2/3 variant | 11456 | WO2018071831 SEQ ID NO: 554 |
| AAV2/3 variant | 11457 | WO2018071831 SEQ ID NO: 555 |
| AAV2/3 variant | 11458 | WO2018071831 SEQ ID NO: 556 |
| AAV2/3 variant | 11459 | WO2018071831 SEQ ID NO: 557 |
| AAV2/3 variant | 11460 | WO2018071831 SEQ ID NO: 558 |
| AAV2/3 variant | 11461 | WO2018071831 SEQ ID NO: 559 |
| AAV2/3 variant | 11462 | WO2018071831 SEQ ID NO: 560 |
| AAV2/3 variant | 11463 | WO2018071831 SEQ ID NO: 561 |
| AAV2/3 variant | 11464 | WO2018071831 SEQ ID NO: 562 |
| AAV2/3 variant | 11465 | WO2018071831 SEQ ID NO: 563 |
| AAV2/3 variant | 11466 | WO2018071831 SEQ ID NO: 564 |
| AAV2/3 variant | 11467 | WO2018071831 SEQ ID NO: 565 |
| AAV2/3 variant | 11468 | WO2018071831 SEQ ID NO: 566 |
| AAV2/3 variant | 11469 | WO2018071831 SEQ ID NO: 567 |
| AAV2/3 variant | 11470 | WO2018071831 SEQ ID NO: 568 |
| AAV2/3 variant | 11471 | WO2018071831 SEQ ID NO: 569 |
| AAV2/3 variant | 11472 | WO2018071831 SEQ ID NO: 570 |
| AAV2/3 variant | 11473 | WO2018071831 SEQ ID NO: 571 |
| AAV2/3 variant | 11474 | WO2018071831 SEQ ID NO: 572 |
| AAV2/3 variant | 11475 | WO2018071831 SEQ ID NO: 573 |
| AAV2/3 variant | 11476 | WO2018071831 SEQ ID NO: 574 |
| AAV2/3 variant | 11477 | WO2018071831 SEQ ID NO: 575 |
| AAV2/3 variant | 11478 | WO2018071831 SEQ ID NO: 576 |
| AAV2/3 variant | 11479 | WO2018071831 SEQ ID NO: 577 |
| AAV2/3 variant | 11480 | WO2018071831 SEQ ID NO: 578 |
| AAV2/3 variant | 11481 | WO2018071831 SEQ ID NO: 579 |
| AAV2/3 variant | 11482 | WO2018071831 SEQ ID NO: 580 |
| AAV2/3 variant | 11483 | WO2018071831 SEQ ID NO: 581 |
| AAV2/3 variant | 11484 | WO2018071831 SEQ ID NO: 582 |
| AAV2/3 variant | 11485 | WO2018071831 SEQ ID NO: 583 |
| AAV2/3 variant | 11486 | WO2018071831 SEQ ID NO: 584 |
| AAV2/3 variant | 11487 | WO2018071831 SEQ ID NO: 585 |
| AAV2/3 variant | 11488 | WO2018071831 SEQ ID NO: 586 |
| AAV2/3 variant | 11489 | WO2018071831 SEQ ID NO: 587 |
| AAV2/3 variant | 11490 | WO2018071831 SEQ ID NO: 588 |
| AAV2/3 variant | 11491 | WO2018071831 SEQ ID NO: 589 |
| AAV2/3 variant | 11492 | WO2018071831 SEQ ID NO: 590 |
| AAV2/3 variant | 11493 | WO2018071831 SEQ ID NO: 591 |
| AAV2/3 variant | 11494 | WO2018071831 SEQ ID NO: 592 |
| AAV2/3 variant | 11495 | WO2018071831 SEQ ID NO: 593 |
| AAV2/3 variant | 11496 | WO2018071831 SEQ ID NO: 594 |
| AAV2/3 variant | 11497 | WO2018071831 SEQ ID NO: 595 |
| AAV2/3 variant | 11498 | WO2018071831 SEQ ID NO: 596 |
| AAV2/3 variant | 11499 | WO2018071831 SEQ ID NO: 597 |
| AAV2/3 variant | 11500 | WO2018071831 SEQ ID NO: 598 |
| AAV2/3 variant | 11501 | WO2018071831 SEQ ID NO: 599 |
| AAV2/3 variant | 11502 | WO2018071831 SEQ ID NO: 600 |
| AAV2/3 variant | 11503 | WO2018071831 SEQ ID NO: 601 |
| AAV2/3 variant | 11504 | WO2018071831 SEQ ID NO: 602 |
| AAV2/3 variant | 11505 | WO2018071831 SEQ ID NO: 603 |
| AAV2/3 variant | 11506 | WO2018071831 SEQ ID NO: 604 |
| AAV2/3 variant | 11507 | WO2018071831 SEQ ID NO: 605 |
| AAV2/3 variant | 11508 | WO2018071831 SEQ ID NO: 606 |
| AAV2/3 variant | 11509 | WO2018071831 SEQ ID NO: 607 |
| AAV2/3 variant | 11510 | WO2018071831 SEQ ID NO: 608 |
| AAV2/3 variant | 11511 | WO2018071831 SEQ ID NO: 609 |
| AAV2/3 variant | 11512 | WO2018071831 SEQ ID NO: 610 |
| AAV2/3 variant | 11513 | WO2018071831 SEQ ID NO: 611 |
| AAV2/3 variant | 11514 | WO2018071831 SEQ ID NO: 612 |
| AAV2/3 variant | 11515 | WO2018071831 SEQ ID NO: 613 |
| AAV2/3 variant | 11516 | WO2018071831 SEQ ID NO: 614 |
| AAV2/3 variant | 11517 | WO2018071831 SEQ ID NO: 615 |
| AAV2/3 variant | 11518 | WO2018071831 SEQ ID NO: 616 |
| AAV2/3 variant | 11519 | WO2018071831 SEQ ID NO: 617 |
| AAV2/3 variant | 11520 | WO2018071831 SEQ ID NO: 618 |
| AAV2/3 variant | 11521 | WO2018071831 SEQ ID NO: 619 |
| AAV2/3 variant | 11522 | WO2018071831 SEQ ID NO: 620 |
| AAV2/3 variant | 11523 | WO2018071831 SEQ ID NO: 621 |
| AAV2/3 variant | 11524 | WO2018071831 SEQ ID NO: 622 |
| AAV2/3 variant | 11525 | WO2018071831 SEQ ID NO: 623 |
| AAV2/3 variant | 11526 | WO2018071831 SEQ ID NO: 624 |
| AAV2/3 variant | 11527 | WO2018071831 SEQ ID NO: 625 |
| AAV2/3 variant | 11528 | WO2018071831 SEQ ID NO: 626 |
| AAV2/3 variant | 11529 | WO2018071831 SEQ ID NO: 627 |
| AAV2/3 variant | 11530 | WO2018071831 SEQ ID NO: 628 |
| AAV8 Variant | 11531 | WO2018071831 SEQ ID NO: 629 |
| AAV8 Variant | 11532 | WO2018071831 SEQ ID NO: 630 |
| AAV8 Variant | 11533 | WO2018071831 SEQ ID NO: 631 |
| AAV8 Variant | 11534 | WO2018071831 SEQ ID NO: 632 |
| AAV8 Variant | 11535 | WO2018071831 SEQ ID NO: 633 |
| AAV8 Variant | 11536 | WO2018071831 SEQ ID NO: 634 |
| AAV8 Variant | 11537 | WO2018071831 SEQ ID NO: 635 |
| AAV8 Variant | 11538 | WO2018071831 SEQ ID NO: 636 |
| AAV8 Variant | 11539 | WO2018071831 SEQ ID NO: 637 |
| AAV8 Variant | 11540 | WO2018071831 SEQ ID NO: 638 |
| AAV8 Variant | 11541 | WO2018071831 SEQ ID NO: 639 |
| AAV8 Variant | 11542 | WO2018071831 SEQ ID NO: 640 |
| AAV8 Variant | 11543 | WO2018071831 SEQ ID NO: 641 |
| AAV8 Variant | 11544 | WO2018071831 SEQ ID NO: 642 |
| AAV8 Variant | 11545 | WO2018071831 SEQ ID NO: 643 |
| AAV8 Variant | 11546 | WO2018071831 SEQ ID NO: 644 |
| AAV8 Variant | 11547 | WO2018071831 SEQ ID NO: 645 |
| AAV8 Variant | 11548 | WO2018071831 SEQ ID NO: 646 |
| AAV8 Variant | 11549 | WO2018071831 SEQ ID NO: 647 |
| AAV8 Variant | 11550 | WO2018071831 SEQ ID NO: 648 |
| AAV8 Variant | 11551 | WO2018071831 SEQ ID NO: 649 |
| AAV8 Variant | 11552 | WO2018071831 SEQ ID NO: 650 |
| AAV8 Variant | 11553 | WO2018071831 SEQ ID NO: 651 |
| AAV8 Variant | 11554 | WO2018071831 SEQ ID NO: 652 |
| AAV8 Variant | 11555 | WO2018071831 SEQ ID NO: 653 |
| AAV8 Variant | 11556 | WO2018071831 SEQ ID NO: 654 |
| AAV8 Variant | 11557 | WO2018071831 SEQ ID NO: 655 |
| AAV8 Variant | 11558 | WO2018071831 SEQ ID NO: 656 |
| AAV8 Variant | 11559 | WO2018071831 SEQ ID NO: 657 |
| AAV8 Variant | 11560 | WO2018071831 SEQ ID NO: 658 |
| AAV8 Variant | 11561 | WO2018071831 SEQ ID NO: 659 |
| AAV8 Variant | 11562 | WO2018071831 SEQ ID NO: 660 |
| AAV8 Variant | 11563 | WO2018071831 SEQ ID NO: 661 |
| AAV8 Variant | 11564 | WO2018071831 SEQ ID NO: 662 |
| AAV8 Variant | 11565 | WO2018071831 SEQ ID NO: 663 |
| AAV8 Variant | 11566 | WO2018071831 SEQ ID NO: 664 |
| AAV8 Variant | 11567 | WO2018071831 SEQ ID NO: 665 |
| AAV8 Variant | 11568 | WO2018071831 SEQ ID NO: 666 |
| AAV8 Variant | 11569 | WO2018071831 SEQ ID NO: 667 |
| AAV8 Variant | 11570 | WO2018071831 SEQ ID NO: 668 |
| AAV8 Variant | 11571 | WO2018071831 SEQ ID NO: 669 |
| AAV8 Variant | 11572 | WO2018071831 SEQ ID NO: 670 |
| AAV8 Variant | 11573 | WO2018071831 SEQ ID NO: 671 |
| AAV8 Variant | 11574 | WO2018071831 SEQ ID NO: 672 |
| AAV8 Variant | 11575 | WO2018071831 SEQ ID NO: 673 |
| AAV8 Variant | 11576 | WO2018071831 SEQ ID NO: 674 |
| AAV8 Variant | 11577 | WO2018071831 SEQ ID NO: 675 |
| AAV8 Variant | 11578 | WO2018071831 SEQ ID NO: 676 |
| AAV8 Variant | 11579 | WO2018071831 SEQ ID NO: 677 |
| AAV8 Variant | 11580 | WO2018071831 SEQ ID NO: 678 |
| AAV8 Variant | 11581 | WO2018071831 SEQ ID NO: 679 |
| AAV8 Variant | 11582 | WO2018071831 SEQ ID NO: 680 |
| AAV8 Variant | 11583 | WO2018071831 SEQ ID NO: 681 |
| AAV8 Variant | 11584 | WO2018071831 SEQ ID NO: 682 |
| AAV8 Variant | 11585 | WO2018071831 SEQ ID NO: 683 |
| AAV8 Variant | 11586 | WO2018071831 SEQ ID NO: 684 |
| AAV8 Variant | 11587 | WO2018071831 SEQ ID NO: 685 |
| AAV8 Variant | 11588 | WO2018071831 SEQ ID NO: 686 |
| AAV8 Variant | 11589 | WO2018071831 SEQ ID NO: 687 |
| AAV8 Variant | 11590 | WO2018071831 SEQ ID NO: 688 |
| AAV8 Variant | 11591 | WO2018071831 SEQ ID NO: 689 |
| AAV8 Variant | 11592 | WO2018071831 SEQ ID NO: 690 |
| AAV8 Variant | 11593 | WO2018071831 SEQ ID NO: 691 |
| AAV8 Variant | 11594 | WO2018071831 SEQ ID NO: 692 |
| AAV8 Variant | 11595 | WO2018071831 SEQ ID NO: 693 |
| AAV8 Variant | 11596 | WO2018071831 SEQ ID NO: 694 |
| AAV8 Variant | 11597 | WO2018071831 SEQ ID NO: 695 |
| AAV8 Variant | 11598 | WO2018071831 SEQ ID NO: 696 |
| AAV8 Variant | 11599 | WO2018071831 SEQ ID NO: 697 |
| AAV8 Variant | 11600 | WO2018071831 SEQ ID NO: 698 |
| AAV8 Variant | 11601 | WO2018071831 SEQ ID NO: 699 |
| AAV8 Variant | 11602 | WO2018071831 SEQ ID NO: 700 |
| AAV8 Variant | 11603 | WO2018071831 SEQ ID NO: 701 |
| AAV8 Variant | 11604 | WO2018071831 SEQ ID NO: 702 |
| AAV8 Variant | 11605 | WO2018071831 SEQ ID NO: 703 |
| AAV8 Variant | 11606 | WO2018071831 SEQ ID NO: 704 |
| AAV8 Variant | 11607 | WO2018071831 SEQ ID NO: 705 |
| AAV8 Variant | 11608 | WO2018071831 SEQ ID NO: 706 |
| AAV8 Variant | 11609 | WO2018071831 SEQ ID NO: 707 |
| AAV8 Variant | 11610 | WO2018071831 SEQ ID NO: 708 |
| AAV8 Variant | 11611 | WO2018071831 SEQ ID NO: 709 |
| AAV8 Variant | 11612 | WO2018071831 SEQ ID NO: 710 |
| AAV8 Variant | 11613 | WO2018071831 SEQ ID NO: 711 |
| AAV8 Variant | 11614 | WO2018071831 SEQ ID NO: 712 |
| AAV8 Variant | 11615 | WO2018071831 SEQ ID NO: 713 |
| AAV8 Variant | 11616 | WO2018071831 SEQ ID NO: 714 |
| AAV8 Variant | 11617 | WO2018071831 SEQ ID NO: 715 |
| AAV8 Variant | 11618 | WO2018071831 SEQ ID NO: 716 |
| AAV8 Variant | 11619 | WO2018071831 SEQ ID NO: 717 |
| AAV8 Variant | 11620 | WO2018071831 SEQ ID NO: 718 |
| AAV8 Variant | 11621 | WO2018071831 SEQ ID NO: 719 |
| AAV8 Variant | 11622 | WO2018071831 SEQ ID NO: 720 |
| AAV8 Variant | 11623 | WO2018071831 SEQ ID NO: 721 |
| AAV8 Variant | 11624 | WO2018071831 SEQ ID NO: 722 |
| AAV8 Variant | 11625 | WO2018071831 SEQ ID NO: 723 |
| AAV8 Variant | 11626 | WO2018071831 SEQ ID NO: 724 |
| AAV8 Variant | 11627 | WO2018071831 SEQ ID NO: 725 |
| AAV8 Variant | 11628 | WO2018071831 SEQ ID NO: 726 |
| AAV8 Variant | 11629 | WO2018071831 SEQ ID NO: 727 |
| AAV8 Variant | 11630 | WO2018071831 SEQ ID NO: 728 |
| AAV8 Variant | 11631 | WO2018071831 SEQ ID NO: 729 |
| AAV8 Variant | 11632 | WO2018071831 SEQ ID NO: 730 |
| AAV8 Variant | 11633 | WO2018071831 SEQ ID NO: 731 |
| AAV8 Variant | 11634 | WO2018071831 SEQ ID NO: 732 |
| AAV8 Variant | 11635 | WO2018071831 SEQ ID NO: 733 |
| AAV8 Variant | 11636 | WO2018071831 SEQ ID NO: 734 |
| AAV8 Variant | 11637 | WO2018071831 SEQ ID NO: 735 |
| AAV8 Variant | 11638 | WO2018071831 SEQ ID NO: 736 |
| AAV8 Variant | 11639 | WO2018071831 SEQ ID NO: 737 |
| AAV8 Variant | 11640 | WO2018071831 SEQ ID NO: 738 |
| AAV8 Variant | 11641 | WO2018071831 SEQ ID NO: 739 |
| AAV8 Variant | 11642 | WO2018071831 SEQ ID NO: 740 |
| AAV8 Variant | 11643 | WO2018071831 SEQ ID NO: 741 |
| AAV8 Variant | 11644 | WO2018071831 SEQ ID NO: 742 |
| AAV8 Variant | 11645 | WO2018071831 SEQ ID NO: 743 |
| AAV8 Variant | 11646 | WO2018071831 SEQ ID NO: 744 |
| AAV8 Variant | 11647 | WO2018071831 SEQ ID NO: 745 |
| AAV8 Variant | 11648 | WO2018071831 SEQ ID NO: 746 |
| AAV8 Variant | 11649 | WO2018071831 SEQ ID NO: 747 |
| AAV8 Variant | 11650 | WO2018071831 SEQ ID NO: 748 |
| AAV8 Variant | 11651 | WO2018071831 SEQ ID NO: 749 |
| AAV8 Variant | 11652 | WO2018071831 SEQ ID NO: 750 |
| AAV8 Variant | 11653 | WO2018071831 SEQ ID NO: 751 |
| AAV8 Variant | 11654 | WO2018071831 SEQ ID NO: 752 |
| AAV8 Variant | 11655 | WO2018071831 SEQ ID NO: 753 |
| AAV8 Variant | 11656 | WO2018071831 SEQ ID NO: 754 |
| AAV8 Variant | 11657 | WO2018071831 SEQ ID NO: 755 |
| AAV8 Variant | 11658 | WO2018071831 SEQ ID NO: 756 |
| AAV8 Variant | 11659 | WO2018071831 SEQ ID NO: 757 |
| AAV8 Variant | 11660 | WO2018071831 SEQ ID NO: 758 |
| AAV8 Variant | 11661 | WO2018071831 SEQ ID NO: 759 |
| AAV8 Variant | 11662 | WO2018071831 SEQ ID NO: 760 |
| AAV8 Variant | 11663 | WO2018071831 SEQ ID NO: 761 |
| AAV8 Variant | 11664 | WO2018071831 SEQ ID NO: 762 |
| AAV8 Variant | 11665 | WO2018071831 SEQ ID NO: 763 |
| AAV8 Variant | 11666 | WO2018071831 SEQ ID NO: 764 |
| AAV8 Variant | 11667 | WO2018071831 SEQ ID NO: 765 |
| AAV8 Variant | 11668 | WO2018071831 SEQ ID NO: 766 |
| AAV8 Variant | 11669 | WO2018071831 SEQ ID NO: 767 |
| AAV8 Variant | 11670 | WO2018071831 SEQ ID NO: 768 |
| AAV8 Variant | 11671 | WO2018071831 SEQ ID NO: 769 |
| AAV8 Variant | 11672 | WO2018071831 SEQ ID NO: 770 |
| AAV8 Variant | 11673 | WO2018071831 SEQ ID NO: 771 |
| AAV8 Variant | 11674 | WO2018071831 SEQ ID NO: 772 |
| AAV8 Variant | 11675 | WO2018071831 SEQ ID NO: 773 |
| AAV8 Variant | 11676 | WO2018071831 SEQ ID NO: 774 |
| AAV8 Variant | 11677 | WO2018071831 SEQ ID NO: 775 |
| AAV8 Variant | 11678 | WO2018071831 SEQ ID NO: 776 |
| AAV8 Variant | 11679 | WO2018071831 SEQ ID NO: 777 |
| AAV8 Variant | 11680 | WO2018071831 SEQ ID NO: 778 |
| AAV8 Variant | 11681 | WO2018071831 SEQ ID NO: 779 |
| AAV8 Variant | 11682 | WO2018071831 SEQ ID NO: 780 |
| AAV8 Variant | 11683 | WO2018071831 SEQ ID NO: 781 |
| AAV8 Variant | 11684 | WO2018071831 SEQ ID NO: 782 |
| AAV8 Variant | 11685 | WO2018071831 SEQ ID NO: 783 |
| AAV8 Variant | 11686 | WO2018071831 SEQ ID NO: 784 |
| AAV8 Variant | 11687 | WO2018071831 SEQ ID NO: 785 |
| AAV8 Variant | 11688 | WO2018071831 SEQ ID NO: 786 |
| AAV8 Variant | 11689 | WO2018071831 SEQ ID NO: 787 |
| AAV8 Variant | 11690 | WO2018071831 SEQ ID NO: 788 |
| AAV8 Variant | 11691 | WO2018071831 SEQ ID NO: 789 |
| AAV8 Variant | 11692 | WO2018071831 SEQ ID NO: 790 |
| AAV8 Variant | 11693 | WO2018071831 SEQ ID NO: 791 |
| AAV8 Variant | 11694 | WO2018071831 SEQ ID NO: 792 |
| AAV8 Variant | 11695 | WO2018071831 SEQ ID NO: 793 |
| AAV8 Variant | 11696 | WO2018071831 SEQ ID NO: 794 |
| AAV8 Variant | 11697 | WO2018071831 SEQ ID NO: 795 |
| AAV8 Variant | 11698 | WO2018071831 SEQ ID NO: 796 |
| AAV8 Variant | 11699 | WO2018071831 SEQ ID NO: 797 |
| AAV8 Variant | 11700 | WO2018071831 SEQ ID NO: 798 |
| AAV8 Variant | 11701 | WO2018071831 SEQ ID NO: 799 |
| AAV8 Variant | 11702 | WO2018071831 SEQ ID NO: 800 |
| AAV8 Variant | 11703 | WO2018071831 SEQ ID NO: 801 |
| AAV8 Variant | 11704 | WO2018071831 SEQ ID NO: 802 |
| AAV8 Variant | 11705 | WO2018071831 SEQ ID NO: 803 |
| AAV8 Variant | 11706 | WO2018071831 SEQ ID NO: 804 |
| AAV8 Variant | 11707 | WO2018071831 SEQ ID NO: 805 |
| AAV8 Variant | 11708 | WO2018071831 SEQ ID NO: 806 |
| AAV8 Variant | 11709 | WO2018071831 SEQ ID NO: 807 |
| AAV8 Variant | 11710 | WO2018071831 SEQ ID NO: 808 |
| AAV8 Variant | 11711 | WO2018071831 SEQ ID NO: 809 |
| AAV8 Variant | 11712 | WO2018071831 SEQ ID NO: 810 |
| AAV8 Variant | 11713 | WO2018071831 SEQ ID NO: 811 |
| AAV8 Variant | 11714 | WO2018071831 SEQ ID NO: 812 |
| AAV8 Variant | 11715 | WO2018071831 SEQ ID NO: 813 |
| AAV8 Variant | 11716 | WO2018071831 SEQ ID NO: 814 |
| AAV8 Variant | 11717 | WO2018071831 SEQ ID NO: 815 |
| AAV8 Variant | 11718 | WO2018071831 SEQ ID NO: 816 |
| AAV8 Variant | 11719 | WO2018071831 SEQ ID NO: 817 |
| AAV8 Variant | 11720 | WO2018071831 SEQ ID NO: 818 |
| AAV8 Variant | 11721 | WO2018071831 SEQ ID NO: 819 |
| AAV8 Variant | 11722 | WO2018071831 SEQ ID NO: 820 |
| AAV8 Variant | 11723 | WO2018071831 SEQ ID NO: 821 |
| AAV8 Variant | 11724 | WO2018071831 SEQ ID NO: 822 |
| AAV8 Variant | 11725 | WO2018071831 SEQ ID NO: 823 |
| AAV8 Variant | 11726 | WO2018071831 SEQ ID NO: 824 |
| AAV8 Variant | 11727 | WO2018071831 SEQ ID NO: 825 |
| AAV8 Variant | 11728 | WO2018071831 SEQ ID NO: 826 |
| AAV8 Variant | 11729 | WO2018071831 SEQ ID NO: 827 |
| AAV8 Variant | 11730 | WO2018071831 SEQ ID NO: 828 |
| AAV8 Variant | 11731 | WO2018071831 SEQ ID NO: 829 |
| AAV8 Variant | 11732 | WO2018071831 SEQ ID NO: 830 |
| AAV8 Variant | 11733 | WO2018071831 SEQ ID NO: 831 |
| AAV8 Variant | 11734 | WO2018071831 SEQ ID NO: 832 |
| AAV8 Variant | 11735 | WO2018071831 SEQ ID NO: 833 |
| AAV8 Variant | 11736 | WO2018071831 SEQ ID NO: 834 |
| AAV8 Variant | 11737 | WO2018071831 SEQ ID NO: 835 |
| AAV8 Variant | 11738 | WO2018071831 SEQ ID NO: 836 |
| AAV2 variant | 11739 | WO2018071831 SEQ ID NO: 837 |
| AAV2 variant | 11740 | WO2018071831 SEQ ID NO: 838 |
| AAV2 variant | 11741 | WO2018071831 SEQ ID NO: 839 |
| AAV2 variant | 11742 | WO2018071831 SEQ ID NO: 840 |
| AAV2 variant | 11743 | WO2018071831 SEQ ID NO: 841 |
| AAV2 variant | 11744 | WO2018071831 SEQ ID NO: 842 |
| AAV2 variant | 11745 | WO2018071831 SEQ ID NO: 843 |
| AAV2 variant | 11746 | WO2018071831 SEQ ID NO: 844 |
| AAV2 variant | 11747 | WO2018071831 SEQ ID NO: 845 |
| AAV2 variant | 11748 | WO2018071831 SEQ ID NO: 846 |
| AAV2 variant | 11749 | WO2018071831 SEQ ID NO: 847 |
| AAV2 variant | 11750 | WO2018071831 SEQ ID NO: 848 |
| AAV2 variant | 11751 | WO2018071831 SEQ ID NO: 849 |
| AAV2 variant | 11752 | WO2018071831 SEQ ID NO: 850 |
| AAV2 variant | 11753 | WO2018071831 SEQ ID NO: 851 |
| AAV2 variant | 11754 | WO2018071831 SEQ ID NO: 852 |
| AAV8 B1 | 11755 | WO2018071831 SEQ ID NO: 853 |
| AAV8 B2 | 11756 | WO2018071831 SEQ ID NO: 854 |
| AAV8 B3 | 11757 | WO2018071831 SEQ ID NO: 855 |
| AAV8 B4 | 11758 | WO2018071831 SEQ ID NO: 856 |
| AAV8 B12 | 11759 | WO2018071831 SEQ ID NO: 857 |
| AAV8 B18 | 11760 | WO2018071831 SEQ ID NO: 858 |
| AAV8 B24 | 11761 | WO2018071831 SEQ ID NO: 859 |
| AAV8 B41 | 11762 | WO2018071831 SEQ ID NO: 860 |
| AAV8 B44 | 11763 | WO2018071831 SEQ ID NO: 861 |
| AAV8 B45 | 11764 | WO2018071831 SEQ ID NO: 862 |
| AAV8 B46 | 11765 | WO2018071831 SEQ ID NO: 863 |
| AAV8 B60 | 11766 | WO2018071831 SEQ ID NO: 864 |
| AAV8 B61 | 11767 | WO2018071831 SEQ ID NO: 865 |
| AAV8 B62 | 11768 | WO2018071831 SEQ ID NO: 866 |
| AAV8 B63 | 11769 | WO2018071831 SEQ ID NO: 867 |
| AAV8 B64 | 11770 | WO2018071831 SEQ ID NO: 868 |
| AAV variant | 11771 | WO2018071831 SEQ ID NO: 876 |
| AAV variant | 11772 | WO2018071831 SEQ ID NO: 877 |
| AAV variant | 11773 | WO2018071831 SEQ ID NO: 878 |
| AAV variant | 11774 | WO2018071831 SEQ ID NO: 879 |
| AAV variant | 11775 | WO2018071831 SEQ ID NO: 880 |
| AAV variant | 11776 | WO2018071831 SEQ ID NO: 881 |
| AAV variant | 11777 | WO2018071831 SEQ ID NO: 882 |
| AAV variant | 11778 | WO2018071831 SEQ ID NO: 883 |
| AAV variant | 11779 | WO2018071831 SEQ ID NO: 884 |
| AAV variant | 11780 | WO2018071831 SEQ ID NO: 885 |
| AAV variant | 11781 | WO2018071831 SEQ ID NO: 886 |
| AAV variant | 11782 | WO2018071831 SEQ ID NO: 887 |
| AAV variant | 11783 | WO2018071831 SEQ ID NO: 888 |
| AAV variant | 11784 | WO2018071831 SEQ ID NO: 889 |
| AAV variant | 11785 | WO2018071831 SEQ ID NO: 890 |
| AAV variant | 11786 | WO2018071831 SEQ ID NO: 891 |
| AAV variant | 11787 | WO2018071831 SEQ ID NO: 892 |
| AAV variant | 11788 | WO2018071831 SEQ ID NO: 893 |
| AAV variant | 11789 | WO2018071831 SEQ ID NO: 894 |
| AAV variant | 11790 | WO2018071831 SEQ ID NO: 895 |
| AAV variant | 11791 | WO2018071831 SEQ ID NO: 896 |
| AAV variant | 11792 | WO2018071831 SEQ ID NO: 897 |
| AAV variant | 11793 | WO2018071831 SEQ ID NO: 898 |
| AAV variant | 11794 | WO2018071831 SEQ ID NO: 899 |
| AAV variant | 11795 | WO2018071831 SEQ ID NO: 900 |
| AAV variant | 11796 | WO2018071831 SEQ ID NO: 901 |
| AAV variant | 11797 | WO2018071831 SEQ ID NO: 902 |
| AAV variant | 11798 | WO2018071831 SEQ ID NO: 903 |
| AAV variant | 11799 | WO2018071831 SEQ ID NO: 904 |
| AAV variant | 11800 | WO2018071831 SEQ ID NO: 905 |
| AAV variant | 11801 | WO2018071831 SEQ ID NO: 906 |
| AAV variant | 11802 | WO2018071831 SEQ ID NO: 907 |
| AAV variant | 11803 | WO2018071831 SEQ ID NO: 908 |
| AAV variant | 11804 | WO2018071831 SEQ ID NO: 909 |
| AAV variant | 11805 | WO2018071831 SEQ ID NO: 910 |
| AAV variant | 11806 | WO2018071831 SEQ ID NO: 911 |
| AAV variant | 11807 | WO2018071831 SEQ ID NO: 912 |
| AAV variant | 11808 | WO2018071831 SEQ ID NO: 913 |
| AAV variant | 11809 | WO2018071831 SEQ ID NO: 914 |
| AAV variant | 11810 | WO2018071831 SEQ ID NO: 915 |
| AAV variant | 11811 | WO2018071831 SEQ ID NO: 916 |
| AAV variant | 11812 | WO2018071831 SEQ ID NO: 917 |
| AAV variant | 11813 | WO2018071831 SEQ ID NO: 918 |
| AAV variant | 11814 | WO2018071831 SEQ ID NO: 919 |
| AAV variant | 11815 | WO2018071831 SEQ ID NO: 920 |
| AAV variant | 11816 | WO2018071831 SEQ ID NO: 921 |
| AAV variant | 11817 | WO2018071831 SEQ ID NO: 922 |
| AAV variant | 11818 | WO2018071831 SEQ ID NO: 923 |
| AAV variant | 11819 | WO2018071831 SEQ ID NO: 924 |
| AAV variant | 11820 | WO2018071831 SEQ ID NO: 925 |
| AAV variant | 11821 | WO2018071831 SEQ ID NO: 926 |
| AAV variant | 11822 | WO2018071831 SEQ ID NO: 927 |
| AAV variant | 11823 | WO2018071831 SEQ ID NO: 928 |
| AAV variant | 11824 | WO2018071831 SEQ ID NO: 929 |
| AAV variant | 11825 | WO2018071831 SEQ ID NO: 930 |
| AAV variant | 11826 | WO2018071831 SEQ ID NO: 931 |
| AAV variant | 11827 | WO2018071831 SEQ ID NO: 932 |
| AAV variant | 11828 | WO2018071831 SEQ ID NO: 933 |
| AAV variant | 11829 | WO2018071831 SEQ ID NO: 934 |
| AAV variant | 11830 | WO2018071831 SEQ ID NO: 935 |
| AAV variant | 11831 | WO2018071831 SEQ ID NO: 936 |
| AAV variant | 11832 | WO2018071831 SEQ ID NO: 937 |
| AAV variant | 11833 | WO2018071831 SEQ ID NO: 938 |
| AAV variant | 11834 | WO2018071831 SEQ ID NO: 939 |
| AAV variant | 11835 | WO2018071831 SEQ ID NO: 940 |
| AAV variant | 11836 | WO2018071831 SEQ ID NO: 941 |
| AAV variant | 11837 | WO2018071831 SEQ ID NO: 942 |
| AAV variant | 11838 | WO2018071831 SEQ ID NO: 943 |
| AAV variant | 11839 | WO2018071831 SEQ ID NO: 944 |
| AAV variant | 11840 | WO2018071831 SEQ ID NO: 945 |
| AAV variant | 11841 | WO2018071831 SEQ ID NO: 946 |
| AAV variant | 11842 | WO2018071831 SEQ ID NO: 947 |
| AAV variant | 11843 | WO2018071831 SEQ ID NO: 948 |
| AAV variant | 11844 | WO2018071831 SEQ ID NO: 949 |
| AAV variant | 11845 | WO2018071831 SEQ ID NO: 950 |
| AAV variant | 11846 | WO2018071831 SEQ ID NO: 951 |
| AAV variant | 11847 | WO2018071831 SEQ ID NO: 952 |
| AAV variant | 11848 | WO2018071831 SEQ ID NO: 953 |
| AAV variant | 11849 | WO2018071831 SEQ ID NO: 954 |
| AAV variant | 11850 | WO2018071831 SEQ ID NO: 955 |
| AAV variant | 11851 | WO2018071831 SEQ ID NO: 956 |
| AAV variant | 11852 | WO2018071831 SEQ ID NO: 957 |
| AAV variant | 11853 | WO2018071831 SEQ ID NO: 958 |
| AAV variant | 11854 | WO2018071831 SEQ ID NO: 959 |
| AAV variant | 11855 | WO2018071831 SEQ ID NO: 960 |
| AAV variant | 11856 | WO2018071831 SEQ ID NO: 961 |
| AAV variant | 11857 | WO2018071831 SEQ ID NO: 962 |
| AAV variant | 11858 | WO2018071831 SEQ ID NO: 963 |
| AAV variant | 11859 | WO2018071831 SEQ ID NO: 964 |
| AAV variant | 11860 | WO2018071831 SEQ ID NO: 965 |
| AAV variant | 11861 | WO2018071831 SEQ ID NO: 966 |
| AAV variant | 11862 | WO2018071831 SEQ ID NO: 967 |
| AAV variant | 11863 | WO2018071831 SEQ ID NO: 968 |
| AAV variant | 11864 | WO2018071831 SEQ ID NO: 969 |
| AAV variant | 11865 | WO2018071831 SEQ ID NO: 970 |
| AAV variant | 11866 | WO2018071831 SEQ ID NO: 971 |
| AAV variant | 11867 | WO2018071831 SEQ ID NO: 972 |
| AAV variant | 11868 | WO2018071831 SEQ ID NO: 973 |
| AAV variant | 11869 | WO2018071831 SEQ ID NO: 974 |
| AAV variant | 11870 | WO2018071831 SEQ ID NO: 975 |
| AAV variant | 11871 | WO2018071831 SEQ ID NO: 976 |
| AAV variant | 11872 | WO2018071831 SEQ ID NO: 977 |
| AAV variant | 11873 | WO2018071831 SEQ ID NO: 978 |
| AAV variant | 11874 | WO2018071831 SEQ ID NO: 979 |
| AAV variant | 11875 | WO2018071831 SEQ ID NO: 980 |
| AAV variant | 11876 | WO2018071831 SEQ ID NO: 981 |
| AAV variant | 11877 | WO2018071831 SEQ ID NO: 982 |
| AAV variant | 11878 | WO2018071831 SEQ ID NO: 983 |
| AAV variant | 11879 | WO2018071831 SEQ ID NO: 984 |
| AAV variant | 11880 | WO2018071831 SEQ ID NO: 985 |
| AAV variant | 11881 | WO2018071831 SEQ ID NO: 986 |
| AAV variant | 11882 | WO2018071831 SEQ ID NO: 987 |
| AAV variant | 11883 | WO2018071831 SEQ ID NO: 988 |
| AAV variant | 11884 | WO2018071831 SEQ ID NO: 989 |
| AAV variant | 11885 | WO2018071831 SEQ ID NO: 990 |
| AAV variant | 11886 | WO2018071831 SEQ ID NO: 991 |
| AAV variant | 11887 | WO2018071831 SEQ ID NO: 992 |
| AAV variant | 11888 | WO2018071831 SEQ ID NO: 993 |
| AAV variant | 11889 | WO2018071831 SEQ ID NO: 994 |
| AAV variant | 11890 | WO2018071831 SEQ ID NO: 995 |
| AAV variant | 11891 | WO2018071831 SEQ ID NO: 996 |
| AAV variant | 11892 | WO2018071831 SEQ ID NO: 997 |
| AAV variant | 11893 | WO2018071831 SEQ ID NO: 998 |
| AAV variant | 11894 | WO2018071831 SEQ ID NO: 999 |
| AAV variant | 11895 | WO2018071831 SEQ ID NO: 1000 |
| AAV variant | 11896 | WO2018071831 SEQ ID NO: 1001 |
| AAV variant | 11897 | WO2018071831 SEQ ID NO: 1002 |
| AAV variant | 11898 | WO2018071831 SEQ ID NO: 1003 |
| AAV variant | 11899 | WO2018071831 SEQ ID NO: 1004 |
| AAV variant | 11900 | WO2018071831 SEQ ID NO: 1005 |
| AAV variant | 11901 | WO2018071831 SEQ ID NO: 1006 |
| AAV variant | 11902 | WO2018071831 SEQ ID NO: 1007 |
| AAV variant | 11903 | WO2018071831 SEQ ID NO: 1008 |
| AAV variant | 11904 | WO2018071831 SEQ ID NO: 1009 |
| AAV variant | 11905 | WO2018071831 SEQ ID NO: 1010 |
| AAV variant | 11906 | WO2018071831 SEQ ID NO: 1011 |
| AAV variant | 11907 | WO2018071831 SEQ ID NO: 1012 |
| AAV variant | 11908 | WO2018071831 SEQ ID NO: 1013 |
| AAV variant | 11909 | WO2018071831 SEQ ID NO: 1014 |
| AAV variant | 11910 | WO2018071831 SEQ ID NO: 1015 |
| AAV variant | 11911 | WO2018071831 SEQ ID NO: 1016 |
| AAV variant | 11912 | WO2018071831 SEQ ID NO: 1017 |
| AAV variant | 11913 | WO2018071831 SEQ ID NO: 1018 |
| AAV variant | 11914 | WO2018071831 SEQ ID NO: 1019 |
| AAV variant | 11915 | WO2018071831 SEQ ID NO: 1020 |
| AAV variant | 11916 | WO2018071831 SEQ ID NO: 1021 |
| AAV variant | 11917 | WO2018071831 SEQ ID NO: 1022 |
| AAV variant | 11918 | WO2018071831 SEQ ID NO: 1023 |
| AAV variant | 11919 | WO2018071831 SEQ ID NO: 1024 |
| AAV variant | 11920 | WO2018071831 SEQ ID NO: 1025 |
| AAV variant | 11921 | WO2018071831 SEQ ID NO: 1026 |
| AAV variant | 11922 | WO2018071831 SEQ ID NO: 1027 |
| AAV variant | 11923 | WO2018071831 SEQ ID NO: 1028 |
| AAV variant | 11924 | WO2018071831 SEQ ID NO: 1029 |
| AAV variant | 11925 | WO2018071831 SEQ ID NO: 1030 |
| AAV variant | 11926 | WO2018071831 SEQ ID NO: 1031 |
| AAV variant | 11927 | WO2018071831 SEQ ID NO: 1032 |
| AAV variant | 11928 | WO2018071831 SEQ ID NO: 1033 |
| AAV variant | 11929 | WO2018071831 SEQ ID NO: 1034 |
| AAV variant | 11930 | WO2018071831 SEQ ID NO: 1035 |
| AAV variant | 11931 | WO2018071831 SEQ ID NO: 1036 |
| AAV variant | 11932 | WO2018071831 SEQ ID NO: 1037 |
| AAV variant | 11933 | WO2018071831 SEQ ID NO: 1038 |
| AAV variant | 11934 | WO2018071831 SEQ ID NO: 1039 |
| AAV variant | 11935 | WO2018071831 SEQ ID NO: 1040 |
| AAV variant | 11936 | WO2018071831 SEQ ID NO: 1041 |
| AAV variant | 11937 | WO2018071831 SEQ ID NO: 1042 |
| AAV variant | 11938 | WO2018071831 SEQ ID NO: 1043 |
| AAV variant | 11939 | WO2018071831 SEQ ID NO: 1044 |
| AAV variant | 11940 | WO2018071831 SEQ ID NO: 1045 |
| AAV variant | 11941 | WO2018071831 SEQ ID NO: 1046 |
| AAV variant | 11942 | WO2018071831 SEQ ID NO: 1047 |
| AAV variant | 11943 | WO2018071831 SEQ ID NO: 1048 |
| AAV variant | 11944 | WO2018071831 SEQ ID NO: 1049 |
| AAV variant | 11945 | WO2018071831 SEQ ID NO: 1050 |
| AAV variant | 11946 | WO2018071831 SEQ ID NO: 1051 |
| AAV variant | 11947 | WO2018071831 SEQ ID NO: 1052 |
| AAV variant | 11948 | WO2018071831 SEQ ID NO: 1053 |
| AAV variant | 11949 | WO2018071831 SEQ ID NO: 1054 |
| AAV variant | 11950 | WO2018071831 SEQ ID NO: 1055 |
| AAV variant | 11951 | WO2018071831 SEQ ID NO: 1056 |
| AAV variant | 11952 | WO2018071831 SEQ ID NO: 1057 |
| AAV variant | 11953 | WO2018071831 SEQ ID NO: 1058 |
| AAV variant | 11954 | WO2018071831 SEQ ID NO: 1059 |
| AAV variant | 11955 | WO2018071831 SEQ ID NO: 1060 |
| AAV variant | 11956 | WO2018071831 SEQ ID NO: 1061 |
| AAV variant | 11957 | WO2018071831 SEQ ID NO: 1062 |
| AAV variant | 11958 | WO2018071831 SEQ ID NO: 1063 |
| AAV variant | 11959 | WO2018071831 SEQ ID NO: 1064 |
| AAV variant | 11960 | WO2018071831 SEQ ID NO: 1065 |
| AAV variant | 11961 | WO2018071831 SEQ ID NO: 1066 |
| AAV variant | 11962 | WO2018071831 SEQ ID NO: 1067 |
| AAV variant | 11963 | WO2018071831 SEQ ID NO: 1068 |
| AAV variant | 11964 | WO2018071831 SEQ ID NO: 1069 |
| AAV variant | 11965 | WO2018071831 SEQ ID NO: 1070 |
| AAV variant | 11966 | WO2018071831 SEQ ID NO: 1071 |
| AAV variant | 11967 | WO2018071831 SEQ ID NO: 1072 |
| AAV variant | 11968 | WO2018071831 SEQ ID NO: 1073 |
| AAV variant | 11969 | WO2018071831 SEQ ID NO: 1074 |
| AAV variant | 11970 | WO2018071831 SEQ ID NO: 1075 |
| AAV variant | 11971 | WO2018071831 SEQ ID NO: 1076 |
| AAV variant | 11972 | WO2018071831 SEQ ID NO: 1077 |
| AAV variant | 11973 | WO2018071831 SEQ ID NO: 1078 |
| AAV variant | 11974 | WO2018071831 SEQ ID NO: 1079 |
| AAV variant | 11975 | WO2018071831 SEQ ID NO: 1080 |
| AAV variant | 11976 | WO2018071831 SEQ ID NO: 1081 |
| AAV variant | 11977 | WO2018071831 SEQ ID NO: 1082 |
| AAV variant | 11978 | WO2018071831 SEQ ID NO: 1083 |
| AAV variant | 11979 | WO2018071831 SEQ ID NO: 1084 |
| AAV variant | 11980 | WO2018071831 SEQ ID NO: 1085 |
| AAV variant | 11981 | WO2018071831 SEQ ID NO: 1086 |
| AAV variant | 11982 | WO2018071831 SEQ ID NO: 1087 |
| AAV variant | 11983 | WO2018071831 SEQ ID NO: 1088 |
| AAV variant | 11984 | WO2018071831 SEQ ID NO: 1089 |
| AAV variant | 11985 | WO2018071831 SEQ ID NO: 1090 |
| AAV variant | 11986 | WO2018071831 SEQ ID NO: 1091 |
| AAV variant | 11987 | WO2018071831 SEQ ID NO: 1092 |
| AAV variant | 11988 | WO2018071831 SEQ ID NO: 1093 |
| AAV variant | 11989 | WO2018071831 SEQ ID NO: 1094 |
| AAV variant | 11990 | WO2018071831 SEQ ID NO: 1095 |
| AAV variant | 11991 | WO2018071831 SEQ ID NO: 1096 |
| AAV variant | 11992 | WO2018071831 SEQ ID NO: 1097 |
| AAV variant | 11993 | WO2018071831 SEQ ID NO: 1098 |
| AAV variant | 11994 | WO2018071831 SEQ ID NO: 1099 |
| AAV variant | 11995 | WO2018071831 SEQ ID NO: 1100 |
| AAV variant | 11996 | WO2018071831 SEQ ID NO: 1101 |
| AAV variant | 11997 | WO2018071831 SEQ ID NO: 1102 |
| AAV variant | 11998 | WO2018071831 SEQ ID NO: 1103 |
| AAV variant | 11999 | WO2018071831 SEQ ID NO: 1104 |
| AAV variant | 12000 | WO2018071831 SEQ ID NO: 1105 |
| AAV variant | 12001 | WO2018071831 SEQ ID NO: 1106 |
| AAV variant | 12002 | WO2018071831 SEQ ID NO: 1107 |
| AAV variant | 12003 | WO2018071831 SEQ ID NO: 1108 |
| AAV variant | 12004 | WO2018071831 SEQ ID NO: 1109 |
| AAV variant | 12005 | WO2018071831 SEQ ID NO: 1110 |
| AAV variant | 12006 | WO2018071831 SEQ ID NO: 1111 |
| AAV variant | 12007 | WO2018071831 SEQ ID NO: 1112 |
| AAV variant | 12008 | WO2018071831 SEQ ID NO: 1113 |
| AAV variant | 12009 | WO2018071831 SEQ ID NO: 1114 |
| AAV variant | 12010 | WO2018071831 SEQ ID NO: 1115 |
| AAV variant | 12011 | WO2018071831 SEQ ID NO: 1116 |
| AAV variant | 12012 | WO2018071831 SEQ ID NO: 1117 |
| AAV variant | 12013 | WO2018071831 SEQ ID NO: 1118 |
| AAV variant | 12014 | WO2018071831 SEQ ID NO: 1119 |
| AAV variant | 12015 | WO2018071831 SEQ ID NO: 1120 |
| AAV variant | 12016 | WO2018071831 SEQ ID NO: 1121 |
| AAV variant | 12017 | WO2018071831 SEQ ID NO: 1122 |
| AAV variant | 12018 | WO2018071831 SEQ ID NO: 1123 |
| AAV variant | 12019 | WO2018071831 SEQ ID NO: 1124 |
| AAV variant | 12020 | WO2018071831 SEQ ID NO: 1125 |
| AAV variant | 12021 | WO2018071831 SEQ ID NO: 1126 |
| AAV variant | 12022 | WO2018071831 SEQ ID NO: 1127 |
| AAV variant | 12023 | WO2018071831 SEQ ID NO: 1128 |
| AAV variant | 12024 | WO2018071831 SEQ ID NO: 1129 |
| AAV variant | 12025 | WO2018071831 SEQ ID NO: 1130 |
| AAV variant | 12026 | WO2018071831 SEQ ID NO: 1131 |
| AAV variant | 12027 | WO2018071831 SEQ ID NO: 1132 |
| AAV variant | 12028 | WO2018071831 SEQ ID NO: 1133 |
| AAV variant | 12029 | WO2018071831 SEQ ID NO: 1134 |
| AAV variant | 12030 | WO2018071831 SEQ ID NO: 1135 |
| AAV variant | 12031 | WO2018071831 SEQ ID NO: 1136 |
| AAV variant | 12032 | WO2018071831 SEQ ID NO: 1137 |
| AAV variant | 12033 | WO2018071831 SEQ ID NO: 1138 |
| AAV variant | 12034 | WO2018071831 SEQ ID NO: 1139 |
| AAV variant | 12035 | WO2018071831 SEQ ID NO: 1140 |
| AAV variant | 12036 | WO2018071831 SEQ ID NO: 1141 |
| AAV variant | 12037 | WO2018071831 SEQ ID NO: 1142 |
| AAV variant | 12038 | WO2018071831 SEQ ID NO: 1143 |
| AAV variant | 12039 | WO2018071831 SEQ ID NO: 1144 |
| AAV variant | 12040 | WO2018071831 SEQ ID NO: 1145 |
| AAV variant | 12041 | WO2018071831 SEQ ID NO: 1146 |
| AAV variant | 12042 | WO2018071831 SEQ ID NO: 1147 |
| AAV variant | 12043 | WO2018071831 SEQ ID NO: 1148 |
| AAV variant | 12044 | WO2018071831 SEQ ID NO: 1149 |
| AAV variant | 12045 | WO2018071831 SEQ ID NO: 1150 |
| AAV variant | 12046 | WO2018071831 SEQ ID NO: 1151 |
| AAV variant | 12047 | WO2018071831 SEQ ID NO: 1152 |
| AAV variant | 12048 | WO2018071831 SEQ ID NO: 1153 |
| AAV variant | 12049 | WO2018071831 SEQ ID NO: 1154 |
| AAV variant | 12050 | WO2018071831 SEQ ID NO: 1155 |
| AAV variant | 12051 | WO2018071831 SEQ ID NO: 1156 |
| AAV variant | 12052 | WO2018071831 SEQ ID NO: 1157 |
| AAV variant | 12053 | WO2018071831 SEQ ID NO: 1158 |
| AAV variant | 12054 | WO2018071831 SEQ ID NO: 1159 |
| AAV variant | 12055 | WO2018071831 SEQ ID NO: 1160 |
| AAV variant | 12056 | WO2018071831 SEQ ID NO: 1161 |
| AAV variant | 12057 | WO2018071831 SEQ ID NO: 1162 |
| AAV variant | 12058 | WO2018071831 SEQ ID NO: 1163 |
| AAV variant | 12059 | WO2018071831 SEQ ID NO: 1164 |
| AAV variant | 12060 | WO2018071831 SEQ ID NO: 1165 |
| AAV variant | 12061 | WO2018071831 SEQ ID NO: 1166 |
| AAV variant | 12062 | WO2018071831 SEQ ID NO: 1167 |
| AAV variant | 12063 | WO2018071831 SEQ ID NO: 1168 |
| AAV variant | 12064 | WO2018071831 SEQ ID NO: 1169 |
| AAV variant | 12065 | WO2018071831 SEQ ID NO: 1170 |
| AAV variant | 12066 | WO2018071831 SEQ ID NO: 1171 |
| AAV variant | 12067 | WO2018071831 SEQ ID NO: 1172 |
| AAV variant | 12068 | WO2018071831 SEQ ID NO: 1173 |
| AAV variant | 12069 | WO2018071831 SEQ ID NO: 1174 |
| AAV variant | 12070 | WO2018071831 SEQ ID NO: 1175 |
| AAV variant | 12071 | WO2018071831 SEQ ID NO: 1176 |
| AAV variant | 12072 | WO2018071831 SEQ ID NO: 1177 |
| AAV variant | 12073 | WO2018071831 SEQ ID NO: 1178 |
| AAV variant | 12074 | WO2018071831 SEQ ID NO: 1179 |
| AAV variant | 12075 | WO2018071831 SEQ ID NO: 1180 |
| AAV variant | 12076 | WO2018071831 SEQ ID NO: 1181 |
| AAV variant | 12077 | WO2018071831 SEQ ID NO: 1182 |
| AAV variant | 12078 | WO2018071831 SEQ ID NO: 1183 |
| AAV variant | 12079 | WO2018071831 SEQ ID NO: 1184 |
| AAV variant | 12080 | WO2018071831 SEQ ID NO: 1185 |
| AAV variant | 12081 | WO2018071831 SEQ ID NO: 1186 |
| AAV variant | 12082 | WO2018071831 SEQ ID NO: 1187 |
| AAV variant | 12083 | WO2018071831 SEQ ID NO: 1188 |
| AAV variant | 12084 | WO2018071831 SEQ ID NO: 1189 |
| AAV variant | 12085 | WO2018071831 SEQ ID NO: 1190 |
| AAV variant | 12086 | WO2018071831 SEQ ID NO: 1191 |
| AAV variant | 12067 | WO2018071831 SEQ ID NO: 1192 |
| AAV variant | 12088 | WO2018071831 SEQ ID NO: 1193 |
| AAV variant | 12089 | WO2018071831 SEQ ID NO: 1194 |
| AAV variant | 12090 | WO2018071831 SEQ ID NO: 1195 |
| AAV variant | 12091 | WO2018071831 SEQ ID NO: 1196 |
| AAV variant | 12092 | WO2018071831 SEQ ID NO: 1197 |
| AAV variant | 12093 | WO2018071831 SEQ ID NO: 1198 |
| AAV variant | 12094 | WO2018071831 SEQ ID NO: 1199 |
| AAV variant | 12095 | WO2018071831 SEQ ID NO: 1200 |
| AAV variant | 12096 | WO2018071831 SEQ ID NO: 1201 |
| AAV variant | 12097 | WO2018071831 SEQ ID NO: 1202 |
| AAV variant | 12098 | WO2018071831 SEQ ID NO: 1203 |
| AAV variant | 12099 | WO2018071831 SEQ ID NO: 1204 |
| AAV variant | 12100 | WO2018071831 SEQ ID NO: 1205 |
| AAV variant | 12101 | WO2018071831 SEQ ID NO: 1206 |
| AAV variant | 12102 | WO2018071831 SEQ ID NO: 1207 |
| AAV variant | 12103 | WO2018071831 SEQ ID NO: 1208 |
| AAV variant | 12104 | WO2018071831 SEQ ID NO: 1209 |
| AAV variant | 12105 | WO2018071831 SEQ ID NO: 1210 |
| AAV variant | 12106 | WO2018071831 SEQ ID NO: 1211 |
| AAV variant | 12107 | WO2018071831 SEQ ID NO: 1212 |
| AAV variant | 12108 | WO2018071831 SEQ ID NO: 1213 |
| AAV variant | 12109 | WO2018071831 SEQ ID NO: 1214 |
| AAV variant | 12110 | WO2018071831 SEQ ID NO: 1215 |
| AAV variant | 12111 | WO2018071831 SEQ ID NO: 1216 |
| AAV variant | 12112 | WO2018071831 SEQ ID NO: 1217 |
| AAV variant | 12113 | WO2018071831 SEQ ID NO: 1218 |
| AAV variant | 12114 | WO2018071831 SEQ ID NO: 1219 |
| AAV variant | 12115 | WO2018071831 SEQ ID NO: 1220 |
| AAV variant | 12116 | WO2018071831 SEQ ID NO: 1221 |
| AAV variant | 12117 | WO2018071831 SEQ ID NO: 1222 |
| AAV variant | 12118 | WO2018071831 SEQ ID NO: 1223 |
| AAV variant | 12119 | WO2018071831 SEQ ID NO: 1224 |
| AAV variant | 12120 | WO2018071831 SEQ ID NO: 1225 |
| AAV variant | 12121 | WO2018071831 SEQ ID NO: 1226 |
| AAV variant | 12122 | WO2018071831 SEQ ID NO: 1227 |
| AAV variant | 12123 | WO2018071831 SEQ ID NO: 1228 |
| AAV variant | 12124 | WO2018071831 SEQ ID NO: 1229 |
| AAV variant | 12125 | WO2018071831 SEQ ID NO: 1230 |
| AAV variant | 12126 | WO2018071831 SEQ ID NO: 1231 |
| AAV variant | 12127 | WO2018071831 SEQ ID NO: 1232 |
| AAV variant | 12128 | WO2018071831 SEQ ID NO: 1233 |
| AAV variant | 12129 | WO2018071831 SEQ ID NO: 1234 |
| AAV variant | 12130 | WO2018071831 SEQ ID NO: 1235 |
| AAV variant | 12131 | WO2018071831 SEQ ID NO: 1236 |
| AAV variant | 12132 | WO2018071831 SEQ ID NO: 1237 |
| AAV variant | 12133 | WO2018071831 SEQ ID NO: 1238 |
| AAV variant | 12134 | WO2018071831 SEQ ID NO: 1239 |
| AAV variant | 12135 | WO2018071831 SEQ ID NO: 1240 |
| AAV variant | 12136 | WO2018071831 SEQ ID NO: 1241 |
| AAV variant | 12137 | WO2018071831 SEQ ID NO: 1242 |
| AAV variant | 12138 | WO2018071831 SEQ ID NO: 1243 |
| AAV variant | 12139 | WO2018071831 SEQ ID NO: 1244 |
| AAV variant | 12140 | WO2018071831 SEQ ID NO: 1245 |
| AAV variant | 12141 | WO2018071831 SEQ ID NO: 1246 |
| AAV variant | 12142 | WO2018071831 SEQ ID NO: 1247 |
| AAV variant | 12143 | WO2018071831 SEQ ID NO: 1248 |
| AAV variant | 12144 | WO2018071831 SEQ ID NO: 1249 |
| AAV variant | 12145 | WO2018071831 SEQ ID NO: 1250 |
| AAV variant | 12146 | WO2018071831 SEQ ID NO: 1251 |
| AAV variant | 12147 | WO2018071831 SEQ ID NO: 1252 |
| AAV variant | 12148 | WO2018071831 SEQ ID NO: 1253 |
| AAV variant | 12149 | WO2018071831 SEQ ID NO: 1254 |
| AAV variant | 12150 | WO2018071831 SEQ ID NO: 1255 |
| AAV variant | 12151 | WO2018071831 SEQ ID NO: 1256 |
| AAV variant | 12152 | WO2018071831 SEQ ID NO: 1257 |
| AAV variant | 12153 | WO2018071831 SEQ ID NO: 1258 |
| AAV variant | 12154 | WO2018071831 SEQ ID NO: 1259 |
| AAV variant | 12155 | WO2018071831 SEQ ID NO: 1260 |
| AAV variant | 12156 | WO2018071831 SEQ ID NO: 1261 |
| AAV variant | 12157 | WO2018071831 SEQ ID NO: 1262 |
| AAV variant | 12158 | WO2018071831 SEQ ID NO: 1263 |
| AAV variant | 12159 | WO2018071831 SEQ ID NO: 1264 |
| AAV variant | 12160 | WO2018071831 SEQ ID NO: 1265 |
| AAV variant | 12161 | WO2018071831 SEQ ID NO: 1266 |
| AAV variant | 12162 | WO2018071831 SEQ ID NO: 1267 |
| AAV variant | 12163 | WO2018071831 SEQ ID NO: 1268 |
| AAV variant | 12164 | WO2018071831 SEQ ID NO: 1269 |
| AAV variant | 12165 | WO2018071831 SEQ ID NO: 1270 |
| AAV variant | 12166 | WO2018071831 SEQ ID NO: 1271 |
| AAV variant | 12167 | WO2018071831 SEQ ID NO: 1272 |
| AAV variant | 12168 | WO2018071831 SEQ ID NO: 1273 |
| AAV variant | 12169 | WO2018071831 SEQ ID NO: 1274 |
| AAV variant | 12170 | WO2018071831 SEQ ID NO: 1275 |
| AAV variant | 12171 | WO2018071831 SEQ ID NO: 1276 |
| AAV variant | 12172 | WO2018071831 SEQ ID NO: 1277 |
| AAV variant | 12173 | WO2018071831 SEQ ID NO: 1278 |
| AAV variant | 12174 | WO2018071831 SEQ ID NO: 1279 |
| AAV variant | 12175 | WO2018071831 SEQ ID NO: 1280 |
| AAV variant | 12176 | WO2018071831 SEQ ID NO: 1281 |
| AAV variant | 12177 | WO2018071831 SEQ ID NO: 1282 |
| AAV variant | 12178 | WO2018071831 SEQ ID NO: 1283 |
| AAV variant | 12179 | WO2018071831 SEQ ID NO: 1284 |
| AAV variant | 12180 | WO2018071831 SEQ ID NO: 1285 |
| AAV variant | 12181 | WO2018071831 SEQ ID NO: 1286 |
| AAV variant | 12182 | WO2018071831 SEQ ID NO: 1287 |
| AAV variant | 12183 | WO2018071831 SEQ ID NO: 1288 |
| AAV variant | 12184 | WO2018071831 SEQ ID NO: 1289 |
| AAV variant | 12185 | WO2018071831 SEQ ID NO: 1290 |
| AAV variant | 12186 | WO2018071831 SEQ ID NO: 1291 |
| AAV variant | 12187 | WO2018071831 SEQ ID NO: 1292 |
| AAV variant | 12188 | WO2018071831 SEQ ID NO: 1293 |
| AAV variant | 12189 | WO2018071831 SEQ ID NO: 1294 |
| AAV variant | 12190 | WO2018071831 SEQ ID NO: 1295 |
| AAV variant | 12191 | WO2018071831 SEQ ID NO: 1296 |
| AAV variant | 12192 | WO2018071831 SEQ ID NO: 1297 |
| AAV variant | 12193 | WO2018071831 SEQ ID NO: 1298 |
| AAV variant | 12194 | WO2018071831 SEQ ID NO: 1299 |
| AAV variant | 12195 | WO2018071831 SEQ ID NO: 1300 |
| AAV variant | 12196 | WO2018071831 SEQ ID NO: 1301 |
| AAV variant | 12197 | WO2018071831 SEQ ID NO: 1302 |
| AAV variant | 12198 | WO2018071831 SEQ ID NO: 1303 |
| AAV variant | 12199 | WO2018071831 SEQ ID NO: 1304 |
| AAV variant | 12200 | WO2018071831 SEQ ID NO: 1305 |
| AAV variant | 12201 | WO2018071831 SEQ ID NO: 1306 |
| AAV variant | 12202 | WO2018071831 SEQ ID NO: 1307 |
| AAV variant | 12203 | WO2018071831 SEQ ID NO: 1308 |
| AAV variant | 12204 | WO2018071831 SEQ ID NO: 1309 |
| AAV variant | 12205 | WO2018071831 SEQ ID NO: 1310 |
| AAV variant | 12206 | WO2018071831 SEQ ID NO: 1311 |
| AAV variant | 12207 | WO2018071831 SEQ ID NO: 1312 |
| AAV variant | 12208 | WO2018071831 SEQ ID NO: 1313 |
| AAV variant | 12209 | WO2018071831 SEQ ID NO: 1314 |
| AAV variant | 12210 | WO2018071831 SEQ ID NO: 1315 |
| AAV variant | 12211 | WO2018071831 SEQ ID NO: 1316 |
| AAV variant | 12212 | WO2018071831 SEQ ID NO: 1317 |
| AAV variant | 12213 | WO2018071831 SEQ ID NO: 1318 |
| AAV variant | 12214 | WO2018071831 SEQ ID NO: 1319 |
| AAV variant | 12215 | WO2018071831 SEQ ID NO: 1320 |
| AAV variant | 12216 | WO2018071831 SEQ ID NO: 1321 |
| AAV variant | 12217 | WO2018071831 SEQ ID NO: 1322 |
| AAV variant | 12218 | WO2018071831 SEQ ID NO: 1323 |
| AAV variant | 12219 | WO2018071831 SEQ ID NO: 1324 |
| AAV variant | 12220 | WO2018071831 SEQ ID NO: 1325 |
| AAV variant | 12221 | WO2018071831 SEQ ID NO: 1326 |
| AAV variant | 12222 | WO2018071831 SEQ ID NO: 1327 |
| AAV variant | 12223 | WO2018071831 SEQ ID NO: 1328 |
| AAV variant | 12224 | WO2018071831 SEQ ID NO: 1329 |
| AAV variant | 12225 | WO2018071831 SEQ ID NO: 1330 |
| AAV variant | 12226 | WO2018071831 SEQ ID NO: 1331 |
| AAV variant | 12227 | WO2018071831 SEQ ID NO: 1332 |
| AAV variant | 12228 | WO2018071831 SEQ ID NO: 1333 |
| AAV variant | 12229 | WO2018071831 SEQ ID NO: 1334 |
| AAV variant | 12230 | WO2018071831 SEQ ID NO: 1335 |
| AAV variant | 12231 | WO2018071831 SEQ ID NO: 1336 |
| AAV variant | 12232 | WO2018071831 SEQ ID NO: 1337 |
| AAV variant | 12233 | WO2018071831 SEQ ID NO: 1338 |
| AAV variant | 12234 | WO2018071831 SEQ ID NO: 1339 |
| AAV variant | 12235 | WO2018071831 SEQ ID NO: 1340 |
| AAV variant | 12236 | WO2018071831 SEQ ID NO: 1341 |
| AAV variant | 12237 | WO2018071831 SEQ ID NO: 1342 |
| AAV variant | 12238 | WO2018071831 SEQ ID NO: 1343 |
| AAV variant | 12239 | WO2018071831 SEQ ID NO: 1344 |
| AAV variant | 12240 | WO2018071831 SEQ ID NO: 1345 |
| AAV variant | 12241 | WO2018071831 SEQ ID NO: 1346 |
| AAV variant | 12242 | WO2018071831 SEQ ID NO: 1347 |
| AAV variant | 12243 | WO2018071831 SEQ ID NO: 1348 |
| AAV variant | 12244 | WO2018071831 SEQ ID NO: 1349 |
| AAV variant | 12245 | WO2018071831 SEQ ID NO: 1350 |
| AAV variant | 12246 | WO2018071831 SEQ ID NO: 1351 |
| AAV variant | 12247 | WO2018071831 SEQ ID NO: 1352 |
| AAV variant | 12248 | WO2018071831 SEQ ID NO: 1353 |
| AAV variant | 12249 | WO2018071831 SEQ ID NO: 1354 |
| AAV variant | 12250 | WO2018071831 SEQ ID NO: 1355 |
| AAV variant | 12251 | WO2018071831 SEQ ID NO: 1356 |
| AAV variant | 12252 | WO2018071831 SEQ ID NO: 1357 |
| AAV variant | 12253 | WO2018071831 SEQ ID NO: 1358 |
| AAV variant | 12254 | WO2018071831 SEQ ID NO: 1359 |
| AAV variant | 12255 | WO2018071831 SEQ ID NO: 1360 |
| AAV variant | 12256 | WO2018071831 SEQ ID NO: 1361 |
| AAV variant | 12257 | WO2018071831 SEQ ID NO: 1362 |
| AAV variant | 12258 | WO2018071831 SEQ ID NO: 1363 |
| AAV variant | 12259 | WO2018071831 SEQ ID NO: 1364 |
| AAV variant | 12260 | WO2018071831 SEQ ID NO: 1365 |
| AAV variant | 12261 | WO2018071831 SEQ ID NO: 1366 |
| AAV variant | 12262 | WO2018071831 SEQ ID NO: 1367 |
| AAV variant | 12263 | WO2018071831 SEQ ID NO: 1368 |
| AAV variant | 12264 | WO2018071831 SEQ ID NO: 1369 |
| AAV variant | 12265 | WO2018071831 SEQ ID NO: 1370 |
| AAV variant | 12266 | WO2018071831 SEQ ID NO: 1371 |
| AAV variant | 12267 | WO2018071831 SEQ ID NO: 1372 |
| AAV variant | 12268 | WO2018071831 SEQ ID NO: 1373 |
| AAV variant | 12269 | WO2018071831 SEQ ID NO: 1374 |
| AAV variant | 12270 | WO2018071831 SEQ ID NO: 1375 |
| AAV variant | 12271 | WO2018071831 SEQ ID NO: 1376 |
| AAV variant | 12272 | WO2018071831 SEQ ID NO: 1377 |
| AAV variant | 12273 | WO2018071831 SEQ ID NO: 1378 |
| AAV variant | 12274 | WO2018071831 SEQ ID NO: 1379 |
| AAV variant | 12275 | WO2018071831 SEQ ID NO: 1380 |
| AAV variant | 12276 | WO2018071831 SEQ ID NO: 1381 |
| AAV variant | 12277 | WO2018071831 SEQ ID NO: 1382 |
| AAV variant | 12278 | WO2018071831 SEQ ID NO: 1383 |
| AAV variant | 12279 | WO2018071831 SEQ ID NO: 1384 |
| AAV variant | 12280 | WO2018071831 SEQ ID NO: 1385 |
| AAV variant | 12281 | WO2018071831 SEQ ID NO: 1386 |
| AAV variant | 12282 | WO2018071831 SEQ ID NO: 1387 |
| AAV variant | 12263 | WO2018071831 SEQ ID NO: 1388 |
| AAV variant | 12284 | WO2018071831 SEQ ID NO: 1389 |
| AAV variant | 12285 | WO2018071831 SEQ ID NO: 1390 |
| AAV variant | 12286 | WO2018071831 SEQ ID NO: 1391 |
| AAV variant | 12287 | WO2018071831 SEQ ID NO: 1392 |
| AAV variant | 12288 | WO2018071831 SEQ ID NO: 1393 |
| AAV variant | 12289 | WO2018071831 SEQ ID NO: 1394 |
| AAV variant | 12290 | WO2018071831 SEQ ID NO: 1395 |
| AAV variant | 12291 | WO2018071831 SEQ ID NO: 1396 |
| AAV variant | 12292 | WO2018071831 SEQ ID NO: 1397 |
| AAV variant | 12293 | WO2018071831 SEQ ID NO: 1398 |
| AAV variant | 12294 | WO2018071831 SEQ ID NO: 1399 |
| AAV variant | 12295 | WO2018071831 SEQ ID NO: 1400 |
| AAV variant | 12296 | WO2018071831 SEQ ID NO: 1401 |
| AAV variant | 12297 | WO2018071831 SEQ ID NO: 1402 |
| AAV variant | 12298 | WO2018071831 SEQ ID NO: 1403 |
| AAV variant | 12299 | WO2018071831 SEQ ID NO: 1404 |
| AAV variant | 12300 | WO2018071831 SEQ ID NO: 1405 |
| AAV variant | 12301 | WO2018071831 SEQ ID NO: 1406 |
| AAV variant | 12302 | WO2018071831 SEQ ID NO: 1407 |
| AAV variant | 12303 | WO2018071831 SEQ ID NO: 1408 |
| AAV variant | 12304 | WO2018071831 SEQ ID NO: 1409 |
| AAV variant | 12305 | WO2018071831 SEQ ID NO: 1410 |
| AAV variant | 12306 | WO2018071831 SEQ ID NO: 1411 |
| AAV variant | 12307 | WO2018071831 SEQ ID NO: 1412 |
| AAV variant | 12308 | WO2018071831 SEQ ID NO: 1413 |
| AAV variant | 12309 | WO2018071831 SEQ ID NO: 1414 |
| AAV variant | 12310 | WO2018071831 SEQ ID NO: 1415 |
| AAV variant | 12311 | WO2018071831 SEQ ID NO: 1416 |
| AAV variant | 12312 | WO2018071831 SEQ ID NO: 1417 |
| AAV variant | 12313 | WO2018071831 SEQ ID NO: 1418 |
| AAV variant | 12314 | WO2018071831 SEQ ID NO: 1419 |
| AAV variant | 12315 | WO2018071831 SEQ ID NO: 1420 |
| AAV variant | 12316 | WO2018071831 SEQ ID NO: 1421 |
| AAV variant | 12317 | WO2018071831 SEQ ID NO: 1422 |
| AAV variant | 12318 | WO2018071831 SEQ ID NO: 1423 |
| AAV variant | 12319 | WO2018071831 SEQ ID NO: 1424 |
| AAV variant | 12320 | WO2018071831 SEQ ID NO: 1425 |
| AAV variant | 12321 | WO2018071831 SEQ ID NO: 1426 |
| AAV variant | 12322 | WO2018071831 SEQ ID NO: 1427 |
| AAV variant | 12323 | WO2018071831 SEQ ID NO: 1428 |
| AAV variant | 12324 | WO2018071831 SEQ ID NO: 1429 |
| AAV variant | 12325 | WO2018071831 SEQ ID NO: 1430 |
| AAV variant | 12326 | WO2018071831 SEQ ID NO: 1431 |
| AAV variant | 12327 | WO2018071831 SEQ ID NO: 1432 |
| AAV variant | 12328 | WO2018071831 SEQ ID NO: 1433 |
| AAV variant | 12329 | WO2018071831 SEQ ID NO: 1434 |
| AAV variant | 12330 | WO2018071831 SEQ ID NO: 1435 |
| AAV variant | 12331 | WO2018071831 SEQ ID NO: 1436 |
| AAV variant | 12332 | WO2018071831 SEQ ID NO: 1437 |
| AAV variant | 12333 | WO2018071831 SEQ ID NO: 1438 |
| AAV variant | 12334 | WO2018071831 SEQ ID NO: 1439 |
| AAV variant | 12335 | WO2018071831 SEQ ID NO: 1440 |
| AAV variant | 12336 | WO2018071831 SEQ ID NO: 1441 |
| AAV variant | 12337 | WO2018071831 SEQ ID NO: 1442 |
| AAV variant | 12338 | WO2018071831 SEQ ID NO: 1443 |
| AAV variant | 12339 | WO2018071831 SEQ ID NO: 1444 |
| AAV variant | 12340 | WO2018071831 SEQ ID NO: 1445 |
| AAV variant | 12341 | WO2018071831 SEQ ID NO: 1446 |
| AAV variant | 12342 | WO2018071831 SEQ ID NO: 1447 |
| AAV variant | 12343 | WO2018071831 SEQ ID NO: 1448 |
| AAV variant | 12344 | WO2018071831 SEQ ID NO: 1449 |
| AAV variant | 12345 | WO2018071831 SEQ ID NO: 1450 |
| AAV variant | 12346 | WO2018071831 SEQ ID NO: 1451 |
| AAV variant | 12347 | WO2018071831 SEQ ID NO: 1452 |
| AAV variant | 12348 | WO2018071831 SEQ ID NO: 1453 |
| AAV variant | 12349 | WO2018071831 SEQ ID NO: 1454 |
| AAV variant | 12350 | WO2018071831 SEQ ID NO: 1455 |
| AAV variant | 12351 | WO2018071831 SEQ ID NO: 1456 |
| AAV variant | 12352 | WO2018071831 SEQ ID NO: 1457 |
| AAV variant | 12353 | WO2018071831 SEQ ID NO: 1458 |
| AAV variant | 12354 | WO2018071831 SEQ ID NO: 1459 |
| AAV variant | 12355 | WO2018071831 SEQ ID NO: 1460 |
| AAV variant | 12356 | WO2018071831 SEQ ID NO: 1461 |
| AAV variant | 12357 | WO2018071831 SEQ ID NO: 1462 |
| AAV variant | 12358 | WO2018071831 SEQ ID NO: 1463 |
| AAV variant | 12359 | WO2018071831 SEQ ID NO: 1464 |
| AAV variant | 12360 | WO2018071831 SEQ ID NO: 1465 |
| AAV variant | 12361 | WO2018071831 SEQ ID NO: 1466 |
| AAV variant | 12362 | WO2018071831 SEQ ID NO: 1467 |
| AAV variant | 12363 | WO2018071831 SEQ ID NO: 1468 |
| AAV variant | 12364 | WO2018071831 SEQ ID NO: 1469 |
| AAV variant | 12365 | WO2018071831 SEQ ID NO: 1470 |
| AAV variant | 12366 | WO2018071831 SEQ ID NO: 1471 |
| AAV variant | 12367 | WO2018071831 SEQ ID NO: 1472 |
| AAV variant | 12368 | WO2018071831 SEQ ID NO: 1473 |
| AAV variant | 12369 | WO2018071831 SEQ ID NO: 1474 |
| AAV variant | 12370 | WO2018071831 SEQ ID NO: 1475 |
| AAV variant | 12371 | WO2018071831 SEQ ID NO: 1476 |
| AAV variant | 12372 | WO2018071831 SEQ ID NO: 1477 |
| AAV variant | 12373 | WO2018071831 SEQ ID NO: 1478 |
| AAV variant | 12374 | WO2018071831 SEQ ID NO: 1479 |
| AAV variant | 12375 | WO2018071831 SEQ ID NO: 1488 |
| AAV variant | 12376 | WO2018071831 SEQ ID NO: 1481 |
| AAV variant | 12377 | WO2018071831 SEQ ID NO: 1482 |
| AAV variant | 12378 | WO2018071831 SEQ ID NO: 1483 |
| AAV variant | 12379 | WO2018071831 SEQ ID NO: 1484 |
| AAV variant | 12380 | WO2018071831 SEQ ID NO: 1485 |
| AAV variant | 12381 | WO2018071831 SEQ ID NO: 1486 |
| AAV variant | 12382 | WO2018071831 SEQ ID NO: 1487 |
| AAV variant | 12383 | WO2018071831 SEQ ID NO: 1488 |
| AAV variant | 12384 | WO2018071831 SEQ ID NO: 1489 |
| AAV variant | 12365 | WO2018071831 SEQ ID NO: 1490 |
| AAV variant | 12386 | WO2018071831 SEQ ID NO: 1491 |
| AAV variant | 12387 | WO2018071831 SEQ ID NO: 1492 |
| AAV variant | 12388 | WO2018071831 SEQ ID NO: 1493 |
| AAV variant | 12389 | WO2018071831 SEQ ID NO: 1494 |
| AAV variant | 12390 | WO2018071831 SEQ ID NO: 1495 |
| AAV variant | 12391 | WO2018071831 SEQ ID NO: 1496 |
| AAV variant | 12392 | WO2018071831 SEQ ID NO: 1497 |
| AAV variant | 12393 | WO2018071831 SEQ ID NO: 1498 |
| AAV variant | 12394 | WO2018071831 SEQ ID NO: 1499 |
| AAV variant | 12395 | WO2018071831 SEQ ID NO: 1500 |
| AAV variant | 12396 | WO2018071831 SEQ ID NO: 1501 |
| AAV variant | 12397 | WO2018071831 SEQ ID NO: 1502 |
| AAV variant | 12398 | WO2018071831 SEQ ID NO: 1503 |
| AAV variant | 12399 | WO2018071831 SEQ ID NO: 1504 |
| AAV variant | 12400 | WO2018071831 SEQ ID NO: 1505 |
| AAV variant | 12401 | WO2018071831 SEQ ID NO: 1506 |
| AAV variant | 12402 | WO2018071831 SEQ ID NO: 1507 |
| AAV variant | 12403 | WO2018071831 SEQ ID NO: 1508 |
| AAV variant | 12404 | WO2018071831 SEQ ID NO: 1509 |
| AAV variant | 12405 | WO2018071831 SEQ ID NO: 1510 |
| AAV variant | 12406 | WO2018071831 SEQ ID NO: 1511 |
| AAV variant | 12407 | WO2018071831 SEQ ID NO: 1512 |
| AAV variant | 12408 | WO2018071831 SEQ ID NO: 1513 |
| AAV variant | 12409 | WO2018071831 SEQ ID NO: 1514 |
| AAV variant | 12410 | WO2018071831 SEQ ID NO: 1515 |
| AAV variant | 12411 | WO2018071831 SEQ ID NO: 1516 |
| AAV variant | 12412 | WO2018071831 SEQ ID NO: 1517 |
| AAV variant | 12413 | WO2018071831 SEQ ID NO: 1518 |
| AAV variant | 12414 | WO2018071831 SEQ ID NO: 1519 |
| AAV variant | 12415 | WO2018071831 SEQ ID NO: 1520 |
| AAV variant | 12416 | WO2018071831 SEQ ID NO: 1521 |
| AAV variant | 12417 | WO2018071831 SEQ ID NO: 1522 |
| AAV variant | 12418 | WO2018071831 SEQ ID NO: 1523 |
| AAV variant | 12419 | WO2018071831 SEQ ID NO: 1524 |
| AAV variant | 12420 | WO2018071831 SEQ ID NO: 1525 |
| AAV variant | 12421 | WO2018071831 SEQ ID NO: 1526 |
| AAV variant | 12422 | WO2018071831 SEQ ID NO: 1527 |
| AAV variant | 12423 | WO2018071831 SEQ ID NO: 1528 |
| AAV variant | 12424 | WO2018071831 SEQ ID NO: 1529 |
| AAV variant | 12425 | WO2018071831 SEQ ID NO: 1530 |
| AAV variant | 12426 | WO2018071831 SEQ ID NO: 1531 |
| AAV variant | 12427 | WO2018071831 SEQ ID NO: 1532 |
| AAV variant | 12428 | WO2018071831 SEQ ID NO: 1533 |
| AAV variant | 12429 | WO2018071831 SEQ ID NO: 1534 |
| AAV variant | 12430 | WO2018071831 SEQ ID NO: 1535 |
| AAV variant | 12431 | WO2018071831 SEQ ID NO: 1536 |
| AAV variant | 12432 | WO2018071831 SEQ ID NO: 1537 |
| AAV variant | 12433 | WO2018071831 SEQ ID NO: 1538 |
| AAV variant | 12434 | WO2018071831 SEQ ID NO: 1539 |
| AAV variant | 12435 | WO2018071831 SEQ ID NO: 1540 |
| AAV variant | 12436 | WO2018071831 SEQ ID NO: 1541 |
| AAV variant | 12437 | WO2018071831 SEQ ID NO: 1542 |
| AAV variant | 12438 | WO2018071831 SEQ ID NO: 1543 |
| AAV variant | 12439 | WO2018071831 SEQ ID NO: 1544 |
| AAV variant | 12440 | WO2018071831 SEQ ID NO: 1545 |
| AAV variant | 12441 | WO2018071831 SEQ ID NO: 1546 |
| AAV variant | 12442 | WO2018071831 SEQ ID NO: 1547 |
| AAV variant | 12443 | WO2018071831 SEQ ID NO: 1548 |
| AAV variant | 12444 | WO2018071831 SEQ ID NO: 1549 |
| AAV variant | 12445 | WO2018071831 SEQ ID NO: 1550 |
| AAV variant | 12446 | WO2018071831 SEQ ID NO: 1551 |
| AAV variant | 12447 | WO2018071831 SEQ ID NO: 1552 |
| AAV variant | 12448 | WO2018071831 SEQ ID NO: 1553 |
| AAV variant | 12449 | WO2018071831 SEQ ID NO: 1554 |
| AAV variant | 12450 | WO2018071831 SEQ ID NO: 1555 |
| AAV variant | 12451 | WO2018071831 SEQ ID NO: 1556 |
| AAV variant | 12452 | WO2018071831 SEQ ID NO: 1557 |
| AAV variant | 12453 | WO2018071831 SEQ ID NO: 1558 |
| AAV variant | 12454 | WO2018071831 SEQ ID NO: 1559 |
| AAV variant | 12455 | WO2018071831 SEQ ID NO: 1560 |
| AAV variant | 12456 | WO2018071831 SEQ ID NO: 1561 |
| AAV variant | 12457 | WO2018071831 SEQ ID NO: 1562 |
| AAV variant | 12458 | WO2018071831 SEQ ID NO: 1563 |
| AAV variant | 12459 | WO2018071831 SEQ ID NO: 1564 |
| AAV variant | 12460 | WO2018071831 SEQ ID NO: 1565 |
| AAV variant | 12461 | WO2018071831 SEQ ID NO: 1566 |
| AAV variant | 12462 | WO2018071831 SEQ ID NO: 1567 |
| AAV variant | 12463 | WO2018071831 SEQ ID NO: 1568 |
| AAV variant | 12464 | WO2018071831 SEQ ID NO: 1569 |
| AAV variant | 12465 | WO2018071831 SEQ ID NO: 1570 |
| AAV variant | 12466 | WO2018071831 SEQ ID NO: 1571 |
| AAV variant | 12467 | WO2018071831 SEQ ID NO: 1572 |
| AAV variant | 12468 | WO2018071831 SEQ ID NO: 1573 |
| AAV variant | 12469 | WO2018071831 SEQ ID NO: 1574 |
| AAV variant | 12470 | WO2018071831 SEQ ID NO: 1575 |
| AAV variant | 12471 | WO2018071831 SEQ ID NO: 1576 |
| AAV variant | 12472 | WO2018071831 SEQ ID NO: 1577 |
| AAV variant | 12473 | WO2018071831 SEQ ID NO: 1578 |
| AAV variant | 12474 | WO2018071831 SEQ ID NO: 1579 |
| AAV variant | 12475 | WO2018071831 SEQ ID NO: 1580 |
| AAV variant | 12476 | WO2018071831 SEQ ID NO: 1581 |
| AAV variant | 12477 | WO2018071831 SEQ ID NO: 1582 |
| AAV variant | 12478 | WO2018071831 SEQ ID NO: 1583 |
| AAV variant | 12479 | WO2018071831 SEQ ID NO: 1584 |
| AAV variant | 12480 | WO2018071831 SEQ ID NO: 1585 |
| AAV variant | 12481 | WO2018071831 SEQ ID NO: 1586 |
| AAV variant | 12482 | WO2018071831 SEQ ID NO: 1587 |
| AAV variant | 12483 | WO2018071831 SEQ ID NO: 1588 |
| AAV variant | 12484 | WO2018071831 SEQ ID NO: 1589 |
| AAV variant | 12485 | WO2018071831 SEQ ID NO: 1590 |
| AAV variant | 12486 | WO2018071831 SEQ ID NO: 1591 |
| AAV variant | 12487 | WO2018071831 SEQ ID NO: 1592 |
| AAV variant | 12488 | WO2018071831 SEQ ID NO: 1593 |
| AAV variant | 12489 | WO2018071831 SEQ ID NO: 1594 |
| AAV variant | 12490 | WO2018071831 SEQ ID NO: 1595 |
| AAV variant | 12491 | WO2018071831 SEQ ID NO: 1596 |
| AAV variant | 12492 | WO2018071831 SEQ ID NO: 1597 |
| AAV variant | 12493 | WO2018071831 SEQ ID NO: 1598 |
| AAV variant | 12494 | WO2018071831 SEQ ID NO: 1599 |
| AAV variant | 12495 | WO2018071831 SEQ ID NO: 1600 |
| AAV variant | 12496 | WO2018071831 SEQ ID NO: 1601 |
| AAV variant | 12497 | WO2018071831 SEQ ID NO: 1602 |
| AAV variant | 12498 | WO2018071831 SEQ ID NO: 1603 |
| AAV variant | 12499 | WO2018071831 SEQ ID NO: 1604 |
| AAV variant | 12560 | WO2018071831 SEQ ID NO: 1605 |
| AAV variant | 12501 | WO2018071831 SEQ ID NO: 1606 |
| AAV variant | 12502 | WO2018071831 SEQ ID NO: 1607 |
| AAV variant | 12503 | WO2018071831 SEQ ID NO: 1608 |
| AAV variant | 12504 | WO2018071831 SEQ ID NO: 1609 |
| AAV variant | 12505 | WO2018071831 SEQ ID NO: 1610 |
| AAV variant | 12506 | WO2018071831 SEQ ID NO: 1611 |
| AAV variant | 12507 | WO2018071831 SEQ ID NO: 1612 |
| AAV variant | 12508 | WO2018071831 SEQ ID NO: 1613 |
| AAV variant | 12509 | WO2018071831 SEQ ID NO: 1614 |
| AAV variant | 12510 | WO2018071831 SEQ ID NO: 1615 |
| AAV variant | 12511 | WO2018071831 SEQ ID NO: 1616 |
| AAV variant | 12512 | WO2018071831 SEQ ID NO: 1617 |
| AAV variant | 12513 | WO2018071831 SEQ ID NO: 1618 |
| AAV variant | 12514 | WO2018071831 SEQ ID NO: 1619 |
| AAV variant | 12515 | WO2018071831 SEQ ID NO: 1620 |
| AAV variant | 12516 | WO2018071831 SEQ ID NO: 1621 |
| AAV variant | 12517 | WO2018071831 SEQ ID NO: 1622 |
| AAV variant | 12518 | WO2018071831 SEQ ID NO: 1623 |
| AAV variant | 12519 | WO2018071831 SEQ ID NO: 1624 |
| AAV variant | 12520 | WO2018071831 SEQ ID NO: 1625 |
| AAV variant | 12521 | WO2018071831 SEQ ID NO: 1626 |
| AAV variant | 12522 | WO2018071831 SEQ ID NO: 1627 |
| AAV variant | 12523 | WO2018071831 SEQ ID NO: 1628 |
| AAV variant | 12524 | WO2018071831 SEQ ID NO: 1629 |
| AAV variant | 12525 | WO2018071831 SEQ ID NO: 1630 |
| AAV variant | 12526 | WO2018071831 SEQ ID NO: 1631 |
| AAV variant | 12527 | WO2018071831 SEQ ID NO: 1632 |
| AAV variant | 12528 | WO2018071831 SEQ ID NO: 1633 |
| AAV variant | 12529 | WO2018071831 SEQ ID NO: 1634 |
| AAV variant | 12530 | WO2018071831 SEQ ID NO: 1635 |
| AAV variant | 12531 | WO2018071831 SEQ ID NO: 1636 |
| AAV variant | 12532 | WO2018071831 SEQ ID NO: 1637 |
| AAV variant | 12533 | WO2018071831 SEQ ID NO: 1638 |
| AAV variant | 12534 | WO2018071831 SEQ ID NO: 1639 |
| AAV variant | 12535 | WO2018071831 SEQ ID NO: 1640 |
| AAV variant | 12536 | WO2018071831 SEQ ID NO: 1641 |
| AAV variant | 12537 | WO2018071831 SEQ ID NO: 1642 |
| AAV variant | 12538 | WO2018071831 SEQ ID NO: 1643 |
| AAV variant | 12539 | WO2018071831 SEQ ID NO: 1644 |
| AAV variant | 12540 | WO2018071831 SEQ ID NO: 1645 |
| AAV variant | 12541 | WO2018071831 SEQ ID NO: 1646 |
| AAV variant | 12542 | WO2018071831 SEQ ID NO: 1647 |
| AAV variant | 12543 | WO2018071831 SEQ ID NO: 1648 |
| AAV variant | 12544 | WO2018071831 SEQ ID NO: 1649 |
| AAV variant | 12545 | WO2018071831 SEQ ID NO: 1650 |
| AAV variant | 12546 | WO2018071831 SEQ ID NO: 1651 |
| AAV variant | 12547 | WO2018071831 SEQ ID NO: 1652 |
| AAV variant | 12548 | WO2018071831 SEQ ID NO: 1653 |
| AAV variant | 12549 | WO2018071831 SEQ ID NO: 1654 |
| AAV variant | 12550 | WO2018071831 SEQ ID NO: 1655 |
| AAV variant | 12551 | WO2018071831 SEQ ID NO: 1656 |
| AAV variant | 12552 | WO2018071831 SEQ ID NO: 1657 |
| AAV variant | 12553 | WO2018071831 SEQ ID NO: 1658 |
| AAV variant | 12554 | WO2018071831 SEQ ID NO: 1659 |
| AAV variant | 12555 | WO2018071831 SEQ ID NO: 1660 |
| AAV variant | 12556 | WO2018071831 SEQ ID NO: 1661 |
| AAV variant | 12557 | WO2018071831 SEQ ID NO: 1662 |
| AAV variant | 12558 | WO2018071831 SEQ ID NO: 1663 |
| AAV variant | 12559 | WO2018071831 SEQ ID NO: 1664 |
| AAV variant | 12560 | WO2018071831 SEQ ID NO: 1665 |
| AAV variant | 12561 | WO2018071831 SEQ ID NO: 1666 |
| AAV variant | 12562 | WO2018071831 SEQ ID NO: 1667 |
| AAV variant | 12563 | WO2018071831 SEQ ID NO: 1668 |
| AAV variant | 12564 | WO2018071831 SEQ ID NO: 1669 |
| AAV variant | 12565 | WO2018071831 SEQ ID NO: 1670 |
| AAV variant | 12566 | WO2018071831 SEQ ID NO: 1671 |
| AAV variant | 12567 | WO2018071831 SEQ ID NO: 1672 |
| AAV variant | 12568 | WO2018071831 SEQ ID NO: 1673 |
| AAV variant | 12569 | WO2018071831 SEQ ID NO: 1674 |
| AAV variant | 12570 | WO2018071831 SEQ ID NO: 1675 |
| AAV variant | 12571 | WO2018071831 SEQ ID NO: 1676 |
| AAV variant | 12572 | WO2018071831 SEQ ID NO: 1677 |
| AAV variant | 12573 | WO2018071831 SEQ ID NO: 1678 |
| AAV variant | 12574 | WO2018071831 SEQ ID NO: 1679 |
| AAV variant | 12575 | WO2018071831 SEQ ID NO: 1680 |
| AAV variant | 12576 | WO2018071831 SEQ ID NO: 1681 |
| AAV variant | 12577 | WO2018071831 SEQ ID NO: 1682 |
| AAV variant | 12578 | WO2018071831 SEQ ID NO: 1683 |
| AAV variant | 12579 | WO2018071831 SEQ ID NO: 1684 |
| AAV variant | 12580 | WO2018071831 SEQ ID NO: 1685 |
| AAV variant | 12581 | WO2018071831 SEQ ID NO: 1686 |
| AAV variant | 12582 | WO2018071831 SEQ ID NO: 1687 |
| AAV variant | 12583 | WO2018071831 SEQ ID NO: 1688 |
| AAV variant | 12584 | WO2018071831 SEQ ID NO: 1689 |
| AAV variant | 12585 | WO2018071831 SEQ ID NO: 1690 |
| AAV variant | 12586 | WO2018071831 SEQ ID NO: 1691 |
| AAV variant | 12587 | WO2018071831 SEQ ID NO: 1692 |
| AAV variant | 12588 | WO2018071831 SEQ ID NO: 1693 |
| AAV variant | 12589 | WO2018071831 SEQ ID NO: 1694 |
| AAV variant | 12590 | WO2018071831 SEQ ID NO: 1695 |
| AAV variant | 12591 | WO2018071831 SEQ ID NO: 1696 |
| AAV variant | 12592 | WO2018071831 SEQ ID NO: 1697 |
| AAV variant | 12593 | WO2018071831 SEQ ID NO: 1698 |
| AAV variant | 12594 | WO2018071831 SEQ ID NO: 1699 |
| AAV variant | 12595 | WO2018071831 SEQ ID NO: 1700 |
| AAV variant | 12596 | WO2018071831 SEQ ID NO: 1701 |
| AAV variant | 12597 | WO2018071831 SEQ ID NO: 1702 |
| AAV variant | 12598 | WO2018071831 SEQ ID NO: 1703 |
| AAV variant | 12599 | WO2018071831 SEQ ID NO: 1704 |
| AAV variant | 12600 | WO2018071831 SEQ ID NO: 1705 |
| AAV variant | 12601 | WO2018071831 SEQ ID NO: 1706 |
| AAV variant | 12602 | WO2018071831 SEQ ID NO: 1707 |
| AAV variant | 12603 | WO2018071831 SEQ ID NO: 1708 |
| AAV variant | 12604 | WO2018071831 SEQ ID NO: 1709 |
| AAV variant | 12605 | WO2018071831 SEQ ID NO: 1710 |
| AAV variant | 12606 | WO2018071831 SEQ ID NO: 1711 |
| AAV variant | 12607 | WO2018071831 SEQ ID NO: 1712 |
| AAV variant | 12608 | WO2018071831 SEQ ID NO: 1713 |
| AAV variant | 12609 | WO2018071831 SEQ ID NO: 1714 |
| AAV variant | 12610 | WO2018071831 SEQ ID NO: 1715 |
| AAV variant | 12611 | WO2018071831 SEQ ID NO: 1716 |
| AAV variant | 12612 | WO2018071831 SEQ ID NO: 1717 |
| AAV variant | 12613 | WO2018071831 SEQ ID NO: 1718 |
| AAV2 variant | 12614 | WO2018071831 SEQ ID NO: 1726 |
| AAV2 variant | 12615 | WO2018071831 SEQ ID NO: 1727 |
| AAV2 variant | 12616 | WO2018071831 SEQ ID NO: 1728 |
| AAV2 variant | 12617 | WO2018071831 SEQ ID NO: 1729 |
| AAV2 variant | 12618 | WO2018071831 SEQ ID NO: 1730 |
| AAV2 variant | 12619 | WO2018071831 SEQ ID NO: 1731 |
| AAV2 variant | 12620 | WO2018071831 SEQ ID NO: 1732 |
| AAV2 variant | 12621 | WO2018071831 SEQ ID NO: 1733 |
| AAV2 variant | 12622 | WO2018071831 SEQ ID NO: 1734 |
| AAV2 variant | 12623 | WO2018071831 SEQ ID NO: 1735 |
| AAV2 variant | 12624 | WO2018071831 SEQ ID NO: 1736 |
| AAV2 variant | 12625 | WO2018071831 SEQ ID NO: 1737 |
| AAV2 variant | 12626 | WO2018071831 SEQ ID NO: 1738 |
| AAV2 variant | 12627 | WO2018071831 SEQ ID NO: 1739 |
| AAV2 variant | 12628 | WO2018071831 SEQ ID NO: 1740 |
| AAV2 variant | 12629 | WO2018071831 SEQ ID NO: 1741 |
| AAV2 variant | 12630 | WO2018071831 SEQ ID NO: 1742 |
| AAV2 variant | 12631 | WO2018071831 SEQ ID NO: 1743 |
| AAV2 variant | 12632 | WO2018071831 SEQ ID NO: 1744 |
| AAV2 variant | 12633 | WO2018071831 SEQ ID NO: 1745 |
| AAV2 variant | 12634 | WO2018071831 SEQ ID NO: 1746 |
| AAV2 variant | 12635 | WO2018071831 SEQ ID NO: 1747 |
| AAV2 variant | 12636 | WO2018071831 SEQ ID NO: 1748 |
| AAV2 variant | 12637 | WO2018071831 SEQ ID NO: 1749 |
| AAV2 variant | 12638 | WO2018071831 SEQ ID NO: 1750 |
| AAV2 variant | 12639 | WO2018071831 SEQ ID NO: 1751 |
| AAV2 variant | 12640 | WO2018071831 SEQ ID NO: 1752 |
| AAV2 variant | 12641 | WO2018071831 SEQ ID NO: 1753 |
| AAV2 variant | 12642 | WO2018071831 SEQ ID NO: 1754 |
| AAV2 variant | 12643 | WO2018071831 SEQ ID NO: 1755 |
| AAV2 variant | 12644 | WO2018071831 SEQ ID NO: 1756 |
| AAV2 variant | 12645 | WO2018071831 SEQ ID NO: 1757 |
| AAV2 variant | 12646 | WO2018071831 SEQ ID NO: 1758 |
| AAV2 variant | 12647 | WO2018071831 SEQ ID NO: 1759 |
| AAV2 variant | 12648 | WO2018071831 SEQ ID NO: 1760 |
| AAV2 variant | 12649 | WO2018071831 SEQ ID NO: 1761 |
| AAV2 variant | 12650 | WO2018071831 SEQ ID NO: 1762 |
| AAV2 variant | 12651 | WO2018071831 SEQ ID NO: 1763 |
| AAV2 variant | 12652 | WO2018071831 SEQ ID NO: 1764 |
| AAV2 variant | 12653 | WO2018071831 SEQ ID NO: 1765 |
| AAV2 variant | 12654 | WO2018071831 SEQ ID NO: 1766 |
| AAV2 variant | 12655 | WO2018071831 SEQ ID NO: 1767 |
| AAV2 variant | 12656 | WO2018071831 SEQ ID NO: 1768 |
| AAV2 variant | 12657 | WO2018071831 SEQ ID NO: 1769 |
| AAV2 variant | 12658 | WO2018071831 SEQ ID NO: 1770 |
| AAV2 variant | 12659 | WO2018071831 SEQ ID NO: 1771 |
| AAV2 variant | 12660 | WO2018071831 SEQ ID NO: 1772 |
| AAV2 variant | 12661 | WO2018071831 SEQ ID NO: 1773 |
| AAV2 variant | 12662 | WO2018071831 SEQ ID NO: 1774 |
| AAV2 variant | 12663 | WO2018071831 SEQ ID NO: 1775 |
| AAV2 variant | 12664 | WO2018071831 SEQ ID NO: 1776 |
| AAV2 variant | 12665 | WO2018071831 SEQ ID NO: 1777 |
| AAV2 variant | 12666 | WO2018071831 SEQ ID NO: 1778 |
| AAV2 variant | 12667 | WO2018071831 SEQ ID NO: 1779 |
| AAV2 variant | 12668 | WO2018071831 SEQ ID NO: 1780 |
| AAV2 variant | 12669 | WO2018071831 SEQ ID NO: 1781 |
| AAV2 variant | 12670 | WO2018071831 SEQ ID NO: 1782 |
| AAV2 variant | 12671 | WO2018071831 SEQ ID NO: 1783 |
| AAV2 variant | 12672 | WO2018071831 SEQ ID NO: 1784 |
| AAV2 variant | 12673 | WO2018071831 SEQ ID NO: 1785 |
| AAV2 variant | 12674 | WO2018071831 SEQ ID NO: 1786 |
| AAV2 variant | 12675 | WO2018071831 SEQ ID NO: 1787 |
| AAV2 variant | 12676 | WO2018071831 SEQ ID NO: 1788 |
| AAV2 variant | 12677 | WO2018071831 SEQ ID NO: 1789 |
| AAV2 variant | 12678 | WO2018071831 SEQ ID NO: 1790 |
| AAV2 variant | 12679 | WO2018071831 SEQ ID NO: 1791 |
| AAV2 variant | 12689 | WO2018071831 SEQ ID NO: 1792 |
| AAV2 variant | 12681 | WO2018071831 SEQ ID NO: 1793 |
| AAV2 variant | 12682 | WO2018071831 SEQ ID NO: 1794 |
| AAV2 variant | 12663 | WO2018071831 SEQ ID NO: 1795 |
| AAV2 variant | 12684 | WO2018071831 SEQ ID NO: 1796 |
| AAV2 variant | 12685 | WO2018071831 SEQ ID NO: 1797 |
| AAV2 variant | 12686 | WO2018071831 SEQ ID NO: 1798 |
| AAV2 variant | 12687 | WO2018071831 SEQ ID NO: 1799 |
| AAV2 variant | 12688 | WO2018071831 SEQ ID NO: 1800 |
| AAV2 variant | 12689 | WO2018071831 SEQ ID NO: 1801 |
| AAV2 variant | 12690 | WO2018071831 SEQ ID NO: 1802 |
| AAV2 variant | 12691 | WO2018071831 SEQ ID NO: 1803 |
| AAV2 variant | 12692 | WO2018071831 SEQ ID NO: 1804 |
| AAV2 variant | 12693 | WO2018071831 SEQ ID NO: 1805 |
| AAV2 variant | 12694 | WO2018071831 SEQ ID NO: 1806 |
| AAV2 variant | 12695 | WO2018071831 SEQ ID NO: 1807 |
| AAV2 variant | 12696 | WO2018071831 SEQ ID NO: 1808 |
| AAV2 variant | 12697 | WO2018071831 SEQ ID NO: 1809 |
| AAV2 variant | 12698 | WO2018071831 SEQ ID NO: 1810 |
| AAV2 variant | 12699 | WO2018071831 SEQ ID NO: 1811 |
| AAV2 variant | 12700 | WO2018071831 SEQ ID NO: 1812 |
| AAV2 variant | 12701 | WO2018071831 SEQ ID NO: 1813 |
| AAV2 variant | 12702 | WO2018071831 SEQ ID NO: 1814 |
| AAV2/3 variant | 12703 | WO2018071831 SEQ ID NO: 1815 |
| AAV2/3 variant | 12764 | WO2018071831 SEQ ID NO: 1816 |
| AAV2/3 variant | 12705 | WO2018071831 SEQ ID NO: 1817 |
| AAV2/3 variant | 12706 | WO2018071831 SEQ ID NO: 1818 |
| AAV2/3 variant | 12707 | WO2018071831 SEQ ID NO: 1819 |
| AAV2/3 variant | 12708 | WO2018071831 SEQ ID NO: 1820 |
| AAV2/3 variant | 12709 | WO2018071831 SEQ ID NO: 1821 |
| AAV2/3 variant | 12710 | WO2018071831 SEQ ID NO: 1822 |
| AAV2/3 variant | 12711 | WO2018071831 SEQ ID NO: 1823 |
| AAV2/3 variant | 12712 | WO2018071831 SEQ ID NO: 1824 |
| AAV2/3 variant | 12713 | WO2018071831 SEQ ID NO: 1825 |
| AAV2/3 variant | 12714 | WO2018071831 SEQ ID NO: 1826 |
| AAV2/3 variant | 12715 | WO2018071831 SEQ ID NO: 1827 |
| AAV2/3 variant | 12716 | WO2018071831 SEQ ID NO: 1828 |
| AAV2/3 variant | 12717 | WO2018071831 SEQ ID NO: 1829 |
| AAV2/3 variant | 12718 | WO2018071831 SEQ ID NO: 1830 |
| AAV2/3 variant | 12719 | WO2018071831 SEQ ID NO: 1831 |
| AAV2/3 variant | 12720 | WO2018071831 SEQ ID NO: 1832 |
| AAV2/3 variant | 12721 | WO2018071831 SEQ ID NO: 1833 |
| AAV2/3 variant | 12722 | WO2018071831 SEQ ID NO: 1834 |
| AAV2/3 variant | 12723 | WO2018071831 SEQ ID NO: 1835 |
| AAV2/3 variant | 12724 | WO2018071831 SEQ ID NO: 1836 |
| AAV2/3 variant | 12725 | WO2018071831 SEQ ID NO: 1837 |
| AAV2/3 variant | 12726 | WO2018071831 SEQ ID NO: 1838 |
| AAV2/3 variant | 12727 | WO2018071831 SEQ ID NO: 1839 |
| AAV2/3 variant | 12728 | WO2018071831 SEQ ID NO: 1840 |
| AAV2/3 variant | 12729 | WO2018071831 SEQ ID NO: 1841 |
| AAV2/3 variant | 12730 | WO2018071831 SEQ ID NO: 1842 |
| AAV2/3 variant | 12731 | WO2018071831 SEQ ID NO: 1843 |
| AAV2/3 variant | 12732 | WO2018071831 SEQ ID NO: 1844 |
| AAV2/3 variant | 12733 | WO2018071831 SEQ ID NO: 1845 |
| AAV2/3 variant | 12734 | WO2018071831 SEQ ID NO: 1846 |
| AAV2/3 variant | 12735 | WO2018071831 SEQ ID NO: 1847 |
| AAV2/3 variant | 12736 | WO2018071831 SEQ ID NO: 1848 |
| AAV2/3 variant | 12737 | WO2018071831 SEQ ID NO: 1849 |
| AAV2/3 variant | 12738 | WO2018071831 SEQ ID NO: 1850 |
| AAV2/3 variant | 12739 | WO2018071831 SEQ ID NO: 1851 |
| AAV2/3 variant | 12740 | WO2018071831 SEQ ID NO: 1852 |
| AAV2/3 variant | 12741 | WO2018071831 SEQ ID NO: 1853 |
| AAV2/3 variant | 12742 | WO2018071831 SEQ ID NO: 1854 |
| AAV2/3 variant | 12743 | WO2018071831 SEQ ID NO: 1855 |
| AAV2/3 variant | 12744 | WO2018071831 SEQ ID NO: 1856 |
| AAV2/3 variant | 12745 | WO2018071831 SEQ ID NO: 1857 |
| AAV2/3 variant | 12746 | WO2018071831 SEQ ID NO: 1858 |
| AAV2/3 variant | 12747 | WO2018071831 SEQ ID NO: 1859 |
| AAV2/3 variant | 12748 | WO2018071831 SEQ ID NO: 1860 |
| AAV2/3 variant | 12749 | WO2018071831 SEQ ID NO: 1861 |
| AAV2/3 variant | 12750 | WO2018071831 SEQ ID NO: 1862 |
| AAV2/3 variant | 12751 | WO2018071831 SEQ ID NO: 1863 |
| AAV2/3 variant | 12752 | WO2018071831 SEQ ID NO: 1864 |
| AAV2/3 variant | 12753 | WO2018071831 SEQ ID NO: 1865 |
| AAV2/3 variant | 12754 | WO2018071831 SEQ ID NO: 1866 |
| AAV2/3 variant | 12755 | WO2018071831 SEQ ID NO: 1867 |
| AAV2/3 variant | 12756 | WO2018071831 SEQ ID NO: 1868 |
| AAV2/3 variant | 12757 | WO2018071831 SEQ ID NO: 1869 |
| AAV2/3 variant | 12758 | WO2018071831 SEQ ID NO: 1870 |
| AAV2/3 variant | 12759 | WO2018071831 SEQ ID NO: 1871 |
| AAV2/3 variant | 12760 | WO2018071831 SEQ ID NO: 1872 |
| AAV2/3 variant | 12761 | WO2018071831 SEQ ID NO: 1873 |
| AAV2/3 variant | 12762 | WO2018071831 SEQ ID NO: 1874 |
| AAV2/3 variant | 12763 | WO2018071831 SEQ ID NO: 1875 |
| AAV2/3 variant | 12764 | WO2018071831 SEQ ID NO: 1876 |
| AAV2/3 variant | 12765 | WO2018071831 SEQ ID NO: 1877 |
| AAV2/3 variant | 12766 | WO2018071831 SEQ ID NO: 1878 |
| AAV2/3 variant | 12767 | WO2018071831 SEQ ID NO: 1879 |
| AAV2/3 variant | 12768 | WO2018071831 SEQ ID NO: 1880 |
| AAV2/3 variant | 12769 | WO2018071831 SEQ ID NO: 1881 |
| AAV2/3 variant | 12770 | WO2018071831 SEQ ID NO: 1882 |
| AAV2/3 variant | 12771 | WO2018071831 SEQ ID NO: 1883 |
| AAV2/3 variant | 12772 | WO2018071831 SEQ ID NO: 1884 |
| AAV2/3 variant | 12773 | WO2018071831 SEQ ID NO: 1885 |
| AAV2/3 variant | 12774 | WO2018071831 SEQ ID NO: 1886 |
| AAV2/3 variant | 12775 | WO2018071831 SEQ ID NO: 1887 |
| AAV2/3 variant | 12776 | WO2018071831 SEQ ID NO: 1888 |
| AAV2/3 variant | 12777 | WO2018071831 SEQ ID NO: 1889 |
| AAV2/3 variant | 12778 | WO2018071831 SEQ ID NO: 1890 |
| AAV2/3 variant | 12779 | WO2018071831 SEQ ID NO: 1891 |
| AAV2/3 variant | 12780 | WO2018071831 SEQ ID NO: 1892 |
| AAV2/3 variant | 12781 | WO2018071831 SEQ ID NO: 1893 |
| AAV2/3 variant | 12782 | WO2018071831 SEQ ID NO: 1894 |
| AAV2/3 variant | 12783 | WO2018071831 SEQ ID NO: 1895 |
| AAV2/3 variant | 12784 | WO2018071831 SEQ ID NO: 1896 |
| AAV2/3 variant | 12785 | WO2018071831 SEQ ID NO: 1897 |
| AAV2/3 variant | 12786 | WO2018071831 SEQ ID NO: 1898 |
| AAV2/3 variant | 12787 | WO2018071831 SEQ ID NO: 1899 |
| AAV2/3 variant | 12788 | WO2018071831 SEQ ID NO: 1900 |
| AAV2/3 variant | 12789 | WO2018071831 SEQ ID NO: 1901 |
| AAV2/3 variant | 12790 | WO2018071831 SEQ ID NO: 1902 |
| AAV2/3 variant | 12791 | WO2018071831 SEQ ID NO: 1903 |
| AAV2/3 variant | 12792 | WO2018071831 SEQ ID NO: 1904 |
| AAV2/3 variant | 12793 | WO2018071831 SEQ ID NO: 1905 |
| AAV2/3 variant | 12794 | WO2018071831 SEQ ID NO: 1906 |
| AAV2/3 variant | 12795 | WO2018071831 SEQ ID NO: 1907 |
| AAV2/3 variant | 12796 | WO2018071831 SEQ ID NO: 1908 |
| AAV2/3 variant | 12797 | WO2018071831 SEQ ID NO: 1909 |
| AAV2/3 variant | 12798 | WO2018071831 SEQ ID NO: 1910 |
| AAV2/3 variant | 12799 | WO2018071831 SEQ ID NO: 1911 |
| AAV2/3 variant | 12800 | WO2018071831 SEQ ID NO: 1912 |
| AAV2/3 variant | 12801 | WO2018071831 SEQ ID NO: 1913 |
| AAV2/3 variant | 12802 | WO2018071831 SEQ ID NO: 1914 |
| AAV2/3 variant | 12803 | WO2018071831 SEQ ID NO: 1915 |
| AAV2/3 variant | 12804 | WO2018071831 SEQ ID NO: 1916 |
| AAV2/3 variant | 12805 | WO2018071831 SEQ ID NO: 1917 |
| AAV2/3 variant | 12806 | WO2018071831 SEQ ID NO: 1918 |
| AAV2/3 variant | 12807 | WO2018071831 SEQ ID NO: 1919 |
| AAV2/3 variant | 12808 | WO2018071831 SEQ ID NO: 1920 |
| AAV2/3 variant | 12809 | WO2018071831 SEQ ID NO: 1921 |
| AAV2/3 variant | 12810 | WO2018071831 SEQ ID NO: 1922 |
| AAV2/3 variant | 12811 | WO2018071831 SEQ ID NO: 1923 |
| AAV2/3 variant | 12812 | WO2018071831 SEQ ID NO: 1924 |
| AAV2/3 variant | 12813 | WO2018071831 SEQ ID NO: 1925 |
| AAV2/3 variant | 12814 | WO2018071831 SEQ ID NO: 1926 |
| AAV2/3 variant | 12815 | WO2018071831 SEQ ID NO: 1927 |
| AAV2/3 variant | 12816 | WO2018071831 SEQ ID NO: 1928 |
| AAV2/3 variant | 12817 | WO2018071831 SEQ ID NO: 1929 |
| AAV2/3 variant | 12818 | WO2018071831 SEQ ID NO: 1930 |
| AAV2/3 variant | 12819 | WO2018071831 SEQ ID NO: 1931 |
| AAV2/3 variant | 12820 | WO2018071831 SEQ ID NO: 1932 |
| AAV2/3 variant | 12821 | WO2018071831 SEQ ID NO: 1933 |
| AAV2/3 variant | 12822 | WO2018071831 SEQ ID NO: 1934 |
| AAV2/3 variant | 12823 | WO2018071831 SEQ ID NO: 1935 |
| AAV2/3 variant | 12824 | WO2018071831 SEQ ID NO: 1936 |
| AAV2/3 variant | 12825 | WO2018071831 SEQ ID NO: 1937 |
| AAV2/3 variant | 12826 | WO2018071831 SEQ ID NO: 1938 |
| AAV2/3 variant | 12827 | WO2018071831 SEQ ID NO: 1939 |
| AAV2/3 variant | 12828 | WO2018071831 SEQ ID NO: 1940 |
| AAV2/3 variant | 12829 | WO2018071831 SEQ ID NO: 1941 |
| AAV2/3 variant | 12830 | WO2018071831 SEQ ID NO: 1942 |
| AAV2/3 variant | 12831 | WO2018071831 SEQ ID NO: 1943 |
| AAV2/3 variant | 12832 | WO2018071831 SEQ ID NO: 1944 |
| AAV2/3 variant | 12833 | WO2018071831 SEQ ID NO: 1945 |
| AAV2/3 variant | 12834 | WO2018071831 SEQ ID NO: 1946 |
| AAV2/3 variant | 12835 | WO2018071831 SEQ ID NO: 1947 |
| AAV2/3 variant | 12836 | WO2018071831 SEQ ID NO: 1948 |
| AAV2/3 variant | 12837 | WO2018071831 SEQ ID NO: 1949 |
| AAV2/3 variant | 12838 | WO2018071831 SEQ ID NO: 1950 |
| AAV2/3 variant | 12839 | WO2018071831 SEQ ID NO: 1951 |
| AAV2/3 variant | 12840 | WO2018071831 SEQ ID NO: 1952 |
| AAV2/3 variant | 12841 | WO2018071831 SEQ ID NO: 1953 |
| AAV2/3 variant | 12842 | WO2018071831 SEQ ID NO: 1954 |
| AAV2/3 variant | 12843 | WO2018071831 SEQ ID NO: 1955 |
| AAV2/3 variant | 12844 | WO2018071831 SEQ ID NO: 1956 |
| AAV2/3 variant | 12845 | WO2018071831 SEQ ID NO: 1957 |
| AAV2/3 variant | 12846 | WO2018071831 SEQ ID NO: 1958 |
| AAV2/3 variant | 12847 | WO2018071831 SEQ ID NO: 1959 |
| AAV2/3 variant | 12848 | WO2018071831 SEQ ID NO: 1960 |
| AAV2/3 variant | 12849 | WO2018071831 SEQ ID NO: 1961 |
| AAV2/3 variant | 12850 | WO2018071831 SEQ ID NO: 1962 |
| AAV2/3 variant | 12851 | WO2018071831 SEQ ID NO: 1963 |
| AAV2/3 variant | 12852 | WO2018071831 SEQ ID NO: 1964 |
| AAV2/3 variant | 12853 | WO2018071831 SEQ ID NO: 1965 |
| AAV2/3 variant | 12854 | WO2018071831 SEQ ID NO: 1966 |
| AAV2/3 variant | 12855 | WO2018071831 SEQ ID NO: 1967 |
| AAV2/3 variant | 12856 | WO2018071831 SEQ ID NO: 1968 |
| AAV2/3 variant | 12857 | WO2018071831 SEQ ID NO: 1969 |
| AAV2/3 variant | 12858 | WO2018071831 SEQ ID NO: 1970 |
| AAV2/3 variant | 12859 | WO2018071831 SEQ ID NO: 1971 |
| AAV2/3 variant | 12860 | WO2018071831 SEQ ID NO: 1972 |
| AAV2/3 variant | 12861 | WO2018071831 SEQ ID NO: 1973 |
| AAV2/3 variant | 12862 | WO2018071831 SEQ ID NO: 1974 |
| AAV2/3 variant | 12863 | WO2018071831 SEQ ID NO: 1975 |
| AAV2/3 variant | 12864 | WO2018071831 SEQ ID NO: 1976 |
| AAV2/3 variant | 12865 | WO2018071831 SEQ ID NO: 1977 |
| AAV2/3 variant | 12866 | WO2018071831 SEQ ID NO: 1978 |
| AAV2/3 variant | 12867 | WO2018071831 SEQ ID NO: 1979 |
| AAV2/3 variant | 12868 | WO2018071831 SEQ ID NO: 1980 |
| AAV2/3 variant | 12869 | WO2018071831 SEQ ID NO: 1981 |
| AAV2/3 variant | 12870 | WO2018071831 SEQ ID NO: 1982 |
| AAV2/3 variant | 12871 | WO2018071831 SEQ ID NO: 1983 |
| AAV2/3 variant | 12872 | WO2018071831 SEQ ID NO: 1984 |
| AAV2/3 variant | 12873 | WO2018071831 SEQ ID NO: 1985 |
| AAV2/3 variant | 12874 | WO2018071831 SEQ ID NO: 1986 |
| AAV2/3 variant | 12875 | WO2018071831 SEQ ID NO: 1987 |
| AAV2/3 variant | 12876 | WO2018071831 SEQ ID NO: 1988 |
| AAV2 variant | 12877 | WO2018071831 SEQ ID NO: 1989 |
| AAV2 variant | 12878 | WO2018071831 SEQ ID NO: 1990 |
| AAV2 variant | 12879 | WO2018071831 SEQ ID NO: 1991 |
| AAV2 variant | 12880 | WO2018071831 SEQ ID NO: 1992 |
| AAV2 variant | 12881 | WO2018071831 SEQ ID NO: 1993 |
| AAV2 variant | 12882 | WO2018071831 SEQ ID NO: 1994 |
| AAV2 variant | 12883 | WO2018071831 SEQ ID NO: 1995 |
| AAV2 variant | 12884 | WO2018071831 SEQ ID NO: 1996 |
| AAV2 variant | 12885 | WO2018071831 SEQ ID NO: 1997 |
| AAV2 variant | 12886 | WO2018071831 SEQ ID NO: 1998 |
| AAV2 variant | 12887 | WO2018071831 SEQ ID NO: 1999 |
| AAV2 variant | 12888 | WO2018071831 SEQ ID NO: 2000 |
| AAV2 variant | 12889 | WO2018071831 SEQ ID NO: 2001 |
| AAV2 variant | 12890 | WO2018071831 SEQ ID NO: 2002 |
| AAV2 variant | 12891 | WO2018071831 SEQ ID NO: 2003 |
| AAV2 variant | 12892 | WO2018071831 SEQ ID NO: 2004 |
| AAV2 variant | 12893 | WO2018071831 SEQ ID NO: 2005 |
| AAV2 variant | 12894 | WO2018071831 SEQ ID NO: 2006 |
| AAV2 variant | 12895 | WO2018071831 SEQ ID NO: 2007 |
| AAV2 variant | 12896 | WO2018071831 SEQ ID NO: 2008 |
| AAV2 variant | 12897 | WO2018071831 SEQ ID NO: 2009 |
| AAV2 variant | 12898 | WO2018071831 SEQ ID NO: 2010 |
| AAV2 variant | 12899 | WO2018071831 SEQ ID NO: 2011 |
| AAV2 variant | 12900 | WO2018071831 SEQ ID NO: 2012 |
| AAV2 variant | 12901 | WO2018071831 SEQ ID NO: 2013 |
| AAV2 variant | 12902 | WO2018071831 SEQ ID NO: 2014 |
| AAV2 variant | 12903 | WO2018071831 SEQ ID NO: 2015 |
| AAV2 variant | 12904 | WO2018071831 SEQ ID NO: 2016 |
| AAV2 variant | 12905 | WO2018071831 SEQ ID NO: 2017 |
| AAV2 variant | 12906 | WO2018071831 SEQ ID NO: 2018 |
| AAV2 variant | 12907 | WO2018071831 SEQ ID NO: 2019 |
| AAV2 variant | 12908 | WO2018071831 SEQ ID NO: 2020 |
| AAV2 variant | 12909 | WO2018071831 SEQ ID NO: 2021 |
| AAV2 variant | 12910 | WO2018071831 SEQ ID NO: 2022 |
| AAV2 variant | 12911 | WO2018071831 SEQ ID NO: 2023 |
| AAV2 variant | 12912 | WO2018071831 SEQ ID NO: 2024 |
| AAV2 variant | 12913 | WO2018071831 SEQ ID NO: 2025 |
| AAV2 variant | 12914 | WO2018071831 SEQ ID NO: 2026 |
| AAV2 variant | 12915 | WO2018071831 SEQ ID NO: 2027 |
| AAV2 variant | 12916 | WO2018071831 SEQ ID NO: 2028 |
| AAV2 variant | 12917 | WO2018071831 SEQ ID NO: 2029 |
| AAV2 variant | 12918 | WO2018071831 SEQ ID NO: 2030 |
| AAV2 variant | 12919 | WO2018071831 SEQ ID NO: 2031 |
| AAV2 variant | 12929 | WO2018071831 SEQ ID NO: 2032 |
| AAV2 variant | 12921 | WO2018071831 SEQ ID NO: 2033 |
| AAV2 variant | 12922 | WO2018071831 SEQ ID NO: 2034 |
| AAV2 variant | 12923 | WO2018071831 SEQ ID NO: 2035 |
| AAV2 variant | 12924 | WO2018071831 SEQ ID NO: 2036 |
| AAV2 variant | 12925 | WO2018071831 SEQ ID NO: 2037 |
| AAV2 variant | 12926 | WO2018071831 SEQ ID NO: 2038 |
| AAV2 variant | 12927 | WO2018071831 SEQ ID NO: 2039 |
| AAV2 variant | 12928 | WO2018071831 SEQ ID NO: 2040 |
| AAV2 variant | 12929 | WO2018071831 SEQ ID NO: 2041 |
| AAV2 variant | 12930 | WO2018071831 SEQ ID NO: 2042 |
| AAV2 variant | 12931 | WO2018071831 SEQ ID NO: 2043 |
| AAV2 variant | 12932 | WO2018071831 SEQ ID NO: 2044 |
| AAV2 variant | 12933 | WO2018071831 SEQ ID NO: 2045 |
| AAV2 variant | 12934 | WO2018071831 SEQ ID NO: 2046 |
| AAV2 variant | 12935 | WO2018071831 SEQ ID NO: 2047 |
| AAV2 variant | 12936 | WO2018071831 SEQ ID NO: 2048 |
| AAV2 variant | 12937 | WO2018071831 SEQ ID NO: 2049 |
| AAV2 variant | 12938 | WO2018071831 SEQ ID NO: 2050 |
| AAV2 variant | 12939 | WO2018071831 SEQ ID NO: 2051 |
| AAV2 variant | 12940 | WO2018071831 SEQ ID NO: 2052 |
| AAV2 variant | 12941 | WO2018071831 SEQ ID NO: 2053 |
| AAV2 variant | 12942 | WO2018071831 SEQ ID NO: 2054 |
| AAV2 variant | 12943 | WO2018071831 SEQ ID NO: 2055 |
| AAV2 variant | 12944 | WO2018071831 SEQ ID NO: 2056 |
| AAV2 variant | 12945 | WO2018071831 SEQ ID NO: 2057 |
| AAV2 variant | 12946 | WO2018071831 SEQ ID NO: 2058 |
| AAV2 variant | 12947 | WO2018071831 SEQ ID NO: 2059 |
| AAV2 variant | 12948 | WO2018071831 SEQ ID NO: 2060 |
| AAV2 variant | 12949 | WO2018071831 SEQ ID NO: 2061 |
| AAV2 variant | 12950 | WO2018071831 SEQ ID NO: 2062 |
| AAV2 variant | 12951 | WO2018071831 SEQ ID NO: 2063 |
| AAV2 variant | 12952 | WO2018071831 SEQ ID NO: 2064 |
| AAV2 variant | 12953 | WO2018071831 SEQ ID NO: 2065 |
| AAV2 variant | 12954 | WO2018071831 SEQ ID NO: 2066 |
| AAV2 variant | 12955 | WO2018071831 SEQ ID NO: 2067 |
| AAV2 variant | 12956 | WO2018071831 SEQ ID NO: 2068 |
| AAV2 variant | 12957 | WO2018071831 SEQ ID NO: 2069 |
| AAV2 variant | 12958 | WO2018071831 SEQ ID NO: 2070 |
| AAV2 variant | 12959 | WO2018071831 SEQ ID NO: 2071 |
| AAV2 variant | 12960 | WO2018071831 SEQ ID NO: 2072 |
| AAV2 variant | 12961 | WO2018071831 SEQ ID NO: 2073 |
| AAV2 variant | 12962 | WO2018071831 SEQ ID NO: 2074 |
| AAV2 variant | 12963 | WO2018071831 SEQ ID NO: 2075 |
| AAV2 variant | 12964 | WO2018071831 SEQ ID NO: 2076 |
| AAV2 variant | 12965 | WO2018071831 SEQ ID NO: 2077 |
| AAV2/3 variant | 12966 | WO2018071831 SEQ ID NO: 2078 |
| AAV2/3 variant | 12967 | WO2018071831 SEQ ID NO: 2079 |
| AAV2/3 variant | 12968 | WO2018071831 SEQ ID NO: 2080 |
| AAV2/3 variant | 12969 | WO2018071831 SEQ ID NO: 2081 |
| AAV2/3 variant | 12970 | WO2018071831 SEQ ID NO: 2082 |
| AAV2/3 variant | 12971 | WO2018071831 SEQ ID NO: 2083 |
| AAV2/3 variant | 12972 | WO2018071831 SEQ ID NO: 2084 |
| AAV2/3 variant | 12973 | WO2018071831 SEQ ID NO: 2085 |
| AAV2/3 variant | 12974 | WO2018071831 SEQ ID NO: 2086 |
| AAV2/3 variant | 12975 | WO2018071831 SEQ ID NO: 2087 |
| AAV2/3 variant | 12976 | WO2018071831 SEQ ID NO: 2088 |
| AAV2/3 variant | 12977 | WO2018071831 SEQ ID NO: 2089 |
| AAV2/3 variant | 12978 | WO2018071831 SEQ ID NO: 2090 |
| AAV2/3 variant | 12979 | WO2018071831 SEQ ID NO: 2091 |
| AAV2/3 variant | 12980 | WO2018071831 SEQ ID NO: 2092 |
| AAV2/3 variant | 12981 | WO2018071831 SEQ ID NO: 2093 |
| AAV2/3 variant | 12982 | WO2018071831 SEQ ID NO: 2094 |
| AAV2/3 variant | 12983 | WO2018071831 SEQ ID NO: 2095 |
| AAV2/3 variant | 12984 | WO2018071831 SEQ ID NO: 2096 |
| AAV2/3 variant | 12985 | WO2018071831 SEQ ID NO: 2097 |
| AAV2/3 variant | 12986 | WO2018071831 SEQ ID NO: 2098 |
| AAV2/3 variant | 12987 | WO2018071831 SEQ ID NO: 2099 |
| AAV2/3 variant | 12988 | WO2018071831 SEQ ID NO: 2100 |
| AAV2/3 variant | 12989 | WO2018071831 SEQ ID NO: 2101 |
| AAV2/3 variant | 12990 | WO2018071831 SEQ ID NO: 2102 |
| AAV2/3 variant | 12991 | WO2018071831 SEQ ID NO: 2103 |
| AAV2/3 variant | 12992 | WO2018071831 SEQ ID NO: 2104 |
| AAV2/3 variant | 12993 | WO2018071831 SEQ ID NO: 2105 |
| AAV2/3 variant | 12994 | WO2018071831 SEQ ID NO: 2106 |
| AAV2/3 variant | 12995 | WO2018071831 SEQ ID NO: 2107 |
| AAV2/3 variant | 12996 | WO2018071831 SEQ ID NO: 2108 |
| AAV2/3 variant | 12997 | WO2018071831 SEQ ID NO: 2109 |
| AAV2/3 variant | 12998 | WO2018071831 SEQ ID NO: 2110 |
| AAV2/3 variant | 12999 | WO2018071831 SEQ ID NO: 2111 |
| AAV2/3 variant | 13000 | WO2018071831 SEQ ID NO: 2112 |
| AAV2/3 variant | 13001 | WO2018071831 SEQ ID NO: 2113 |
| AAV2/3 variant | 13002 | WO2018071831 SEQ ID NO: 2114 |
| AAV2/3 variant | 13003 | WO2018071831 SEQ ID NO: 2115 |
| AAV2/3 variant | 13004 | WO2018071831 SEQ ID NO: 2116 |
| AAV2/3 variant | 13005 | WO2018071831 SEQ ID NO: 2117 |
| AAV2/3 variant | 13006 | WO2018071831 SEQ ID NO: 2118 |
| AAV2/3 variant | 13007 | WO2018071831 SEQ ID NO: 2119 |
| AAV2/3 variant | 13008 | WO2018071831 SEQ ID NO: 2120 |
| AAV2/3 variant | 13009 | WO2018071831 SEQ ID NO: 2121 |
| AAV2/3 variant | 13010 | WO2018071831 SEQ ID NO: 2122 |
| AAV2/3 variant | 13011 | WO2018071831 SEQ ID NO: 2123 |
| AAV2/3 variant | 13012 | WO2018071831 SEQ ID NO: 2124 |
| AAV2/3 variant | 13013 | WO2018071831 SEQ ID NO: 2125 |
| AAV2/3 variant | 13014 | WO2018071831 SEQ ID NO: 2126 |
| AAV2/3 variant | 13015 | WO2018071831 SEQ ID NO: 2127 |
| AAV2/3 variant | 13016 | WO2018071831 SEQ ID NO: 2128 |
| AAV2/3 variant | 13017 | WO2018071831 SEQ ID NO: 2129 |
| AAV2/3 variant | 13018 | WO2018071831 SEQ ID NO: 2130 |
| AAV2/3 variant | 13619 | WO2018071831 SEQ ID NO: 2131 |
| AAV2/3 variant | 13020 | WO2018071831 SEQ ID NO: 2132 |
| AAV2/3 variant | 13021 | WO2018071831 SEQ ID NO: 2133 |
| AAV2/3 variant | 13022 | WO2018071831 SEQ ID NO: 2134 |
| AAV2/3 variant | 13023 | WO2018071831 SEQ ID NO: 2135 |
| AAV2/3 variant | 13024 | WO2018071831 SEQ ID NO: 2136 |
| AAV2/3 variant | 13025 | WO2018071831 SEQ ID NO: 2137 |
| AAV2/3 variant | 13026 | WO2018071831 SEQ ID NO: 2138 |
| AAV2/3 variant | 13027 | WO2018071831 SEQ ID NO: 2139 |
| AAV2/3 variant | 13028 | WO2018071831 SEQ ID NO: 2140 |
| AAV2/3 variant | 13029 | WO2018071831 SEQ ID NO: 2141 |
| AAV2/3 variant | 13030 | WO2018071831 SEQ ID NO: 2142 |
| AAV2/3 variant | 13031 | WO2018071831 SEQ ID NO: 2143 |
| AAV2/3 variant | 13032 | WO2018071831 SEQ ID NO: 2144 |
| AAV2/3 variant | 13033 | WO2018071831 SEQ ID NO: 2145 |
| AAV2/3 variant | 13034 | WO2018071831 SEQ ID NO: 2146 |
| AAV2/3 variant | 13035 | WO2018071831 SEQ ID NO: 2147 |
| AAV2/3 variant | 13036 | WO2018071831 SEQ ID NO: 2148 |
| AAV2/3 variant | 13037 | WO2018071831 SEQ ID NO: 2149 |
| AAV2/3 variant | 13038 | WO2018071831 SEQ ID NO: 2150 |
| AAV2/3 variant | 13039 | WO2018071831 SEQ ID NO: 2151 |
| AAV2/3 variant | 13040 | WO2018071831 SEQ ID NO: 2152 |
| AAV2/3 variant | 13041 | WO2018071831 SEQ ID NO: 2153 |
| AAV2/3 variant | 13042 | WO2018071831 SEQ ID NO: 2154 |
| AAV2/3 variant | 13043 | WO2018071831 SEQ ID NO: 2155 |
| AAV2/3 variant | 13044 | WO2018071831 SEQ ID NO: 2156 |
| AAV2/3 variant | 13045 | WO2018071831 SEQ ID NO: 2157 |
| AAV2/3 variant | 13046 | WO2018071831 SEQ ID NO: 2158 |
| AAV2/3 variant | 13047 | WO2018071831 SEQ ID NO: 2159 |
| AAV2/3 variant | 13048 | WO2018071831 SEQ ID NO: 2160 |
| AAV2/3 variant | 13049 | WO2018071831 SEQ ID NO: 2161 |
| AAV2/3 variant | 13050 | WO2018071831 SEQ ID NO: 2162 |
| AAV2/3 variant | 13051 | WO2018071831 SEQ ID NO: 2163 |
| AAV2/3 variant | 13052 | WO2018071831 SEQ ID NO: 2164 |
| AAV2/3 variant | 13053 | WO2018071831 SEQ ID NO: 2165 |
| AAV2/3 variant | 13054 | WO2018071831 SEQ ID NO: 2166 |
| AAV2/3 variant | 13055 | WO2018071831 SEQ ID NO: 2167 |
| AAV2/3 variant | 13056 | WO2018071831 SEQ ID NO: 2168 |
| AAV2/3 variant | 13057 | WO2018071831 SEQ ID NO: 2169 |
| AAV2/3 variant | 13058 | WO2018071831 SEQ ID NO: 2170 |
| AAV2/3 variant | 13059 | WO2018071831 SEQ ID NO: 2171 |
| AAV2/3 variant | 13060 | WO2018071831 SEQ ID NO: 2172 |
| AAV2/3 variant | 13061 | WO2018071831 SEQ ID NO: 2173 |
| AAV2/3 variant | 13062 | WO2018071831 SEQ ID NO: 2174 |
| AAV2/3 variant | 13063 | WO2018071831 SEQ ID NO: 2175 |
| AAV2/3 variant | 13064 | WO2018071831 SEQ ID NO: 2176 |
| AAV2/3 variant | 13065 | WO2018071831 SEQ ID NO: 2177 |
| AAV2/3 variant | 13066 | WO2018071831 SEQ ID NO: 2178 |
| AAV2/3 variant | 13067 | WO2018071831 SEQ ID NO: 2179 |
| AAV2/3 variant | 13068 | WO2018071831 SEQ ID NO: 2180 |
| AAV2/3 variant | 13069 | WO2018071831 SEQ ID NO: 2181 |
| AAV2/3 variant | 13070 | WO2018071831 SEQ ID NO: 2182 |
| AAV2/3 variant | 13071 | WO2018071831 SEQ ID NO: 2183 |
| AAV2/3 variant | 13072 | WO2018071831 SEQ ID NO: 2184 |
| AAV2/3 variant | 13073 | WO2018071831 SEQ ID NO: 2185 |
| AAV2/3 variant | 13074 | WO2018071831 SEQ ID NO: 2186 |
| AAV2/3 variant | 13075 | WO2018071831 SEQ ID NO: 2187 |
| AAV2/3 variant | 13076 | WO2018071831 SEQ ID NO: 2188 |
| AAV2/3 variant | 13077 | WO2018071831 SEQ ID NO: 2189 |
| AAV2/3 variant | 13078 | WO2018071831 SEQ ID NO: 2190 |
| AAV2/3 variant | 13079 | WO2018071831 SEQ ID NO: 2191 |
| AAV2/3 variant | 13080 | WO2018071831 SEQ ID NO: 2192 |
| AAV2/3 variant | 13081 | WO2018071831 SEQ ID NO: 2193 |
| AAV2/3 variant | 13082 | WO2018071831 SEQ ID NO: 2194 |
| AAV2/3 variant | 13083 | WO2018071831 SEQ ID NO: 2195 |
| AAV2/3 variant | 13064 | WO2018071831 SEQ ID NO: 2196 |
| AAV2/3 variant | 13085 | WO2018071831 SEQ ID NO: 2197 |
| AAV2/3 variant | 13086 | WO2018071831 SEQ ID NO: 2198 |
| AAV2/3 variant | 13087 | WO2018071831 SEQ ID NO: 2199 |
| AAV2/3 variant | 13088 | WO2018071831 SEQ ID NO: 2200 |
| AAV2/3 variant | 13089 | WO2018071831 SEQ ID NO: 2201 |
| AAV2/3 variant | 13090 | WO2018071831 SEQ ID NO: 2202 |
| AAV2/3 variant | 13091 | WO2018071831 SEQ ID NO: 2203 |
| AAV2/3 variant | 13092 | WO2018071831 SEQ ID NO: 2204 |
| AAV2/3 variant | 13093 | WO2018071831 SEQ ID NO: 2205 |
| AAV2/3 variant | 13094 | WO2018071831 SEQ ID NO: 2206 |
| AAV2/3 variant | 13095 | WO2018071831 SEQ ID NO: 2207 |
| AAV2/3 variant | 13096 | WO2018071831 SEQ ID NO: 2208 |
| AAV2/3 variant | 13097 | WO2018071831 SEQ ID NO: 2209 |
| AAV2/3 variant | 13098 | WO2018071831 SEQ ID NO: 2210 |
| AAV2/3 variant | 13099 | WO2018071831 SEQ ID NO: 2211 |
| AAV2/3 variant | 13100 | WO2018071831 SEQ ID NO: 2212 |
| AAV2/3 variant | 13101 | WO2018071831 SEQ ID NO: 2213 |
| AAV2/3 variant | 13102 | WO2018071831 SEQ ID NO: 2214 |
| AAV2/3 variant | 13103 | WO2018071831 SEQ ID NO: 2215 |
| AAV2/3 variant | 13164 | WO2018071831 SEQ ID NO: 2216 |
| AAV2/3 variant | 13105 | WO2018071831 SEQ ID NO: 2217 |
| AAV2/3 variant | 13106 | WO2018071831 SEQ ID NO: 2218 |
| AAV2/3 variant | 13107 | WO2018071831 SEQ ID NO: 2219 |
| AAV2/3 variant | 13108 | WO2018071831 SEQ ID NO: 2220 |
| AAV2/3 variant | 13109 | WO2018071831 SEQ ID NO: 2221 |
| AAV2/3 variant | 13110 | WO2018071831 SEQ ID NO: 2222 |
| AAV2/3 variant | 13111 | WO2018071831 SEQ ID NO: 2223 |
| AAV2/3 variant | 13112 | WO2018071831 SEQ ID NO: 2224 |
| AAV2/3 variant | 13113 | WO2018071831 SEQ ID NO: 2225 |
| AAV2/3 variant | 13114 | WO2018071831 SEQ ID NO: 2226 |
| AAV2/3 variant | 13115 | WO2018071831 SEQ ID NO: 2227 |
| AAV2/3 variant | 13116 | WO2018071831 SEQ ID NO: 2228 |
| AAV2/3 variant | 13117 | WO2018071831 SEQ ID NO: 2229 |
| AAV2/3 variant | 13118 | WO2018071831 SEQ ID NO: 2230 |
| AAV2/3 variant | 13119 | WO2018071831 SEQ ID NO: 2231 |
| AAV2/3 variant | 13120 | WO2018071831 SEQ ID NO: 2232 |
| AAV2/3 variant | 13121 | WO2018071831 SEQ ID NO: 2233 |
| AAV2/3 variant | 13122 | WO2018071831 SEQ ID NO: 2234 |
| AAV2/3 variant | 13123 | WO2018071831 SEQ ID NO: 2235 |
| AAV2/3 variant | 13124 | WO2018071831 SEQ ID NO: 2236 |
| AAV2/3 variant | 13125 | WO2018071831 SEQ ID NO: 2237 |
| AAV2/3 variant | 13126 | WO2018071831 SEQ ID NO: 2238 |
| AAV2/3 variant | 13127 | WO2018071831 SEQ ID NO: 2239 |
| AAV2/3 variant | 13128 | WO2018071831 SEQ ID NO: 2240 |
| AAV2/3 variant | 13129 | WO2018071831 SEQ ID NO: 2241 |
| AAV2/3 variant | 13130 | WO2018071831 SEQ ID NO: 2242 |
| AAV2/3 variant | 13131 | WO2018071831 SEQ ID NO: 2243 |
| AAV2/3 variant | 13132 | WO2018071831 SEQ ID NO: 2244 |
| AAV2/3 variant | 13133 | WO2018071831 SEQ ID NO: 2245 |
| AAV2/3 variant | 13134 | WO2018071831 SEQ ID NO: 2246 |
| AAV2/3 variant | 13135 | WO2018071831 SEQ ID NO: 2247 |
| AAV2/3 variant | 13136 | WO2018071831 SEQ ID NO: 2248 |
| AAV2/3 variant | 13137 | WO2018071831 SEQ ID NO: 2249 |
| AAV2/3 variant | 13138 | WO2018071831 SEQ ID NO: 2250 |
| AAV2/3 variant | 13139 | WO2018071831 SEQ ID NO: 2251 |

In some embodiments, the AAV serotype may be, or may have a sequence as described in International Patent Publication WO2015038959, the contents of which are herein incorporated by reference in their entirety, such as, but not limited to, AAV9 (SEQ ID NO: 2 and 11 of WO2015038958 or SEQ ID NO: 137 and 138 respectively herein), PHP.B (SEQ ID NO: 8 and 9 of WO2015038958, herein SEQ ID NO: 5 and 6), G2B-3.13 (SEQ ID NO: 12 of WO2015038958, herein SEQ ID NO: 7), G21-26 (SEQ ID NO: 13 of WO2015038958, herein SEQ ID NO: 5), TH1.1-32 (SEQ ID NO:14 of WO2015038958, herein SEQ ID NO: 8), TH1.1-35 (SEQ ID NO: 15 of WO2015038958, herein SEQ ID NO: 9) or variants thereof. Further, any of the targeting peptides or amino acid inserts described in WO2015038958, may be inserted into any parent AAV serotype, such as, but not limited to, AAV9 (SEQ ID NO: 137 for the DNA sequence and SEQ ID NO: 138 for the amino acid sequence). In some embodiments, the amino acid insert is inserted between amino acids 586.592 of the parent AAV (e.g., AAV9). In another embodiment, the amino acid insert is inserted between amino acids 588-589 of the parent AAV sequence. The amino acid insert may be, but is not limited to, any of the following amino acid sequences, TLAVPFK (SEQ ID NO: 1 of WO2015033953; herein SEQ ID NO: 1262), KFPVALT (SEQ ID NO: 3 of WO2015038958; herein SEQ ID NO: 1263), LAVPFK (SEQ ID NO: 31 of WO2015038958; herein SEQ ID NO: 1264), AVPFK (SEQ ID NO: 32 of WO2015038958; herein SEQ ID NO: 1265), VPFK (SEQ ID NO: 33 of WO2015038958; herein SEQ ID NO: 11266), TLAVPF (SEQ ID NO: 34 of WO2015038958; herein SEQ ID NO: 1267), TLAVP (SEQ ID NO: 35 of WO2015038958; herein SEQ ID NO: 1268), TLAV (SEQ ID NO: 36 of WO2015038958; herein SEQ ID NO: 1269), SVSKPFL (SEQ ID NO: 28 of WO2015038958; herein SEQ ID NO: 1270), FTLTTPK (SEQ ID NO: 29 of WO2015038953; herein SEQ ID NO: 1271), MNATKNV (SEQ ID NO: 30 of WO2015038958; herein SEQ ID NO: 1272), QSSQTPR(SEQ ID NO: 54 of WO2015038958; herein SEQ ID NO: 1273), ILGTGTS (SEQ ID NO: 55 of WO2015038958; herein SEQ ID NO: 1274), TRTNPEA (SEQ ID NO: 56 of WO2015038958; herein SEQ ID NO: 1275), NGGTSSS (SEQ ID NO: 58 of WO2015038958: herein SEQ ID NO: 1276), or YTLSQGW (SEQ ID NO: 60 of WO2015038958; herein SEQ ID NO: 1277). Non-limiting examples of nucleotide sequences that may encode the amino acid inserts include the following, AAGTTTCCTGTGGCGTTGACT (for SEQ ID NO: 3 of WO2015038958; herein SEQ ID NO: 1278), ACTTTGGCGGTGCCTTTTAAG (SEQ ID NO: 24 and 49 of WO2015038958; herein SEQ ID NO: 1279), AGTGTGAGTAAGCCTTTTTTG (SEQ ID NO: 25 of WO2015038958; herein SEQ ID NO: 1280), TTTACGTTGACGACGCCTAAG (SEQ ID NO: 26 of WO2015038958; herein SEQ ID NO: 1281), ATGAATGCTACGAAGAATGTG (SEQ ID NO: 27 of WO2015038958; herein SEQ ID NO: 1282), CAGTCGTCGCAGACGCCTAGG (SEQ ID NO: 48 of WO2015038958; herein SEQ ID NO: 1283), ATTCTGGGGACTGGTACTTCG (SEQ ID NO: 50 and 52 of WO2015038958; herein SEQ ID NO: 1284), ACGCGGACTAATCCTGAGGCT (SEQ ID NO: 51 of WO2015038958; herein SEQ ID NO: 1285), AATGGGGGGACTAGTAGTTCT (SEQ ID NO: 53 of WO2015038958; herein SEQ ID NO: 1286), or TATACTTTGTCGCAGGGTTGG (SEQ ID NO: 59 of WO2015038958; herein SEQ ID NO: 1287).

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 11), PHP.N (SEQ ID NO: 46 of WO2017100671, herein SEQ ID NO: 4), PHP.S (SEQ ID NO: 47 of WO2017100671, herein SEQ ID NO: 10), or variants thereof. Further, any of the targeting peptides or amino acid inserts described in WO2017100671 may be inserted into any parent AAV serotype, such as, but not limited to, AAV9. In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences, AQTLAVPFKAQ (SEQ ID NO: 1 of WO2017100671; herein SEQ ID NO: 1288), AQSVSKPFLAQ (SEQ ID NO: 2 of WO2017100671; herein SEQ ID NO: 1289), AQFTLTTPKAQ (SEQ ID NO: 3 in the sequence listing of WO2017100671; herein SEQ ID NO: 1290), DGTLAVPFKAQ (SEQ ID NO: 4 in the sequence listing of WO2017100671; herein SEQ ID NO: 1291), ESTLAVPFKAQ (SEQ ID NO: 5 of WO2017100671; herein SEQ ID NO: 1292), GGTLAVPFKAQ (SEQ ID NO: 6 of WO2017100671; herein SEQ ID NO: 1293), AQTLATPFKAQ (SEQ ID NO: 7 and 33 of WO2017100671; herein SEQ ID NO: 1294), ATTLATPFKAQ (SEQ ID NO: 8 of WO2017100671; herein SEQ ID NO: 1295), DGTLATPFKAQ (SEQ ID NO: 9 of WO2017100671; herein SEQ ID NO: 1296), GGTLATPFKAQ (SEQ ID NO: 10 of WO2017100671; herein SEQ ID NO: 1297), SGSLAVPFKAQ (SEQ ID NO: 11 of WO2017100671; herein SEQ ID NO: 1298), AQTLAQPFKAQ (SEQ ID NO: 12 of WO2017100671; herein SEQ ID NO: 1299), AQTLQQPFKAQ (SEQ ID NO: 13 of WO2017100671; herein SEQ ID NO: 1300), AQTLSNPFKAQ (SEQ ID NO: 14 of WO2017100671; herein SEQ ID NO: 1301), AQTLAVPFSNP (SEQ ID NO: 15 of WO2017100671; herein SEQ ID NO: 1302), QGTLAVPFKAQ (SEQ ID NO: 16 of WO2017100671; herein SEQ ID NO: 1303), NQTLAVPFKAQ (SEQ ID NO: 17 of WO2017100671; herein SEQ ID NO: 1304), EGSLAVPFKAQ (SEQ ID NO: 18 of WO2017100671; herein SEQ ID NO: 1305), SGNLAVPFKAQ (SEQ ID NO: 19 of WO2017100671; herein SEQ ID NO: 1306), EGTLAVPFKAQ (SEQ ID NO: 20 of WO2017100671; herein SEQ ID NO: 1307), DSTLAVPFKAQ (SEQ ID NO: 21 in Table 1 of WO2017100671; herein SEQ ID NO: 1308), AVTLAVPFKAQ (SEQ ID NO: 22 of WO2017100671; herein SEQ ID NO: 1309), AQTLSTPFKAQ (SEQ ID NO: 23 of WO2017100671; herein SEQ ID NO: 1310), AQTLPQPFKAQ (SEQ ID NO: 24 and 32 of WO2017100671; herein SEQ ID NO: 1311), AQTLSQPFKAQ (SEQ ID NO: 25 of WO2017100671; herein SEQ ID NO: 1312), AQTLQLPFKAQ (SEQ ID NO: 26 of WO2017100671; herein SEQ ID NO: 1313), AQTLTMPFKAQ (SEQ ID NO: 27, and 34 of WO2017100671 and SEQ ID NO: 35 in the sequence listing of WO2017100671; herein SEQ ID NO: 1314), AQTLTTPFKAQ (SEQ ID NO: 28 of WO2017100671; herein SEQ ID NO: 1315), AQYTLSQGWAQ (SEQ ID NO: 29 of WO2017100671; herein SEQ ID NO: 1316), AQMNATKNVAQ (SEQ ID NO: 30 of WO2017100671; herein SEQ ID NO: 1317), AQVSGGHHSAQ (SEQ ID NO: 31 of WO2017100671; herein SEQ ID NO: 1318), AQTLTAPFKAQ (SEQ ID NO: 35 in Table 1 of WO2017100671; herein SEQ ID NO: 1319), AQTLSKPFKAQ (SEQ ID NO: 36 of WO2017100671; herein SEQ ID NO: 1320), QAVRTSL (SEQ ID NO: 37 of WO2017100671; herein SEQ ID NO: 1321), YTLSQGW (SEQ ID NO: 38 of WO2017100671; herein SEQ ID NO: 1277), LAKERLS (SEQ ID NO: 39 of WO2017100671; herein SEQ ID NO: 1322), TLAVPFK (SEQ ID NO: 40 in the sequence listing of WO2017100671; herein SEQ ID NO: 1262), SVSKPFL (SEQ ID NO: 41 of WO2017100671; herein SEQ ID NO: 1270), FTLTTPK (SEQ ID NO: 42 of WO2017100671; herein SEQ ID NO: 1271), MNSTKNV (SEQ ID NO: 43 of WO2017100671; herein SEQ ID NO: 1323), VSGGHHS (SEQ ID NO: 44 of WO2017100671; herein SEQ ID NO: 1324), SAQTLAVPFKAQAQ (SEQ ID NO: 48 of WO2017100671; herein SEQ ID NO: 1325), SXXXLAVPFKAQAQ (SEQ ID NO: 49 of WO2017100671 wherein X may be any amino acid; herein SEQ ID NO: 1326), SAQXXXVPFKAQAQ (SEQ ID NO: 50 of WO2017100671 wherein X may be any amino acid; herein SEQ ID NO: 1327), SAQTLXXXFKAQAQ (SEQ ID NO: 51 of WO2017100671 wherein X may be any amino acid; herein SEQ ID NO: 1328), SAQTLAVXXXAQAQ (SEQ ID NO: 52 of WO2017100671 wherein X may be any amino acid; herein SEQ ID NO: 1329), SAQTLAVPFXXXAQ (SEQ ID NO: 53 of WO2017100671 wherein X may be any amino acid; herein SEQ ID NO: 1330), TNHQSAQ (SEQ ID NO: 65 of WO2017100671; herein SEQ ID NO: 1331), AQAQTGW (SEQ ID NO: 66 of WO2017100671; herein SEQ ID NO: 1332), DGTLATPFK (SEQ ID NO: 67 of WO2017100671; herein SEQ ID NO: 1333), DGTLATPFKXX (SEQ ID NO: 68 of WO2017100671 wherein X may be any amino acid; herein SEQ ID NO: 1334), LAVPFKAQ (SEQ ID NO: 80 of WO2017100671; herein SEQ ID NO: 1335), VPFKAQ (SEQ ID NO: 81 of WO2017100671; herein SEQ ID NO: 1336), FKAQ (SEQ ID NO: 82 of WO2017100671; herein SEQ ID NO: 1337), AQTLAV (SEQ ID NO: 83 of WO2017100671; herein SEQ ID NO: 1338), AQTLAVPF (SEQ ID NO: 84 of WO2017100671; herein SEQ ID NO: 1339), QAVR (SEQ ID NO: 85 of WO2017100671; herein SEQ ID NO: 1340), AVRT (SEQ ID NO: 86 of WO2017100671; herein SEQ ID NO: 1341), VRTS (SEQ ID NO: 87 of WO2017100671; herein SEQ ID NO: 1342), RTSL (SEQ ID NO: 88 of WO2017100671; herein SEQ ID NO: 1343), QAVRT (SEQ ID NO: 89 of WO2017100671; herein SEQ ID NO: 1344), AVRTS (SEQ ID NO: 90 of WO2017100671; herein SEQ ID NO: 1345), VRTSL (SEQ ID NO: 91 of WO2017100671; herein SEQ ID NO: 1346), QAVRTS (SEQ ID NO: 92 of WO2017100671; herein SEQ ID NO: 1347), or AVRTSL (SEQ ID NO: 93 of WO2017100671; herein SEQ ID NO: 1348).

Non-limiting examples of nucleotide sequences that may encode the amino acid inserts include the following, GATGGGACTTTGGCGGTGCCTTTTAAGGCACAG (SEQ ID NO: 54 of WO2017100671; herein SEQ ID NO: 1349), GATGGGACGTTGGCGGTGCCTTTTAAGGCACAG (SEQ ID NO: 55 of WO2017100671; herein SEQ ID NO: 1350), CAGGCGGTTAGGACGTCTTTG (SEQ ID NO: 56 of WO2017100671; herein SEQ ID NO: 1351), CAGGTCTTCACGGACTCAGACTATCAG (SEQ ID NO: 57 and 78 of WO2017100671; herein SEQ ID NO:1352), CAAGTAAAACCTCTACAAATGTGGTAAAATCG (SEQ ID NO: 58 of WO2017100671; herein SEQ ID NO: 1353), ACTCATCGACCAATACTTGTACTATCTCTCTAGAAC (SEQ ID NO: 59 of WO2017100671; herein SEQ ID NO: 1354), GGAAGTATTCCTTGGTTTTGAACCCA (SEQ ID NO: 60 of WO2017100671; herein SEQ ID NO: 1355), GGTCGCGGTTCTTGTTTGTGGAT (SEQ ID NO: 61 of WO2017100671; herein SEQ ID NO: 1356), CGACCTTGAAGCGCATGAACTCCT (SEQ ID NO: 62 of WO2017100671; herein SEQ ID NO: 1357), GTATTCCTTGGTTTTGACCCACCGGTCTGCGCCTGTGCNNMNNMNNMNNMNNNNMNNTTGGGCACTCTGGTGGTTTGTC (SEQ ID NO: 63 of WO2017100671 wherein N may be A, C, T, or G; herein SEQ ID NO: 1358), GTATTCCTTGGTTTTGAACCCAACCGGTCTGCGCMNNMNNMNNAAGGCACCGCCAAAGTTTG (SEQ ID NO: 69 of WO2017100671 wherein N may be A, C, T, or G; herein SEQ ID NO: 1359), GTATTCCTTGGTTTTGAACCCAACCGGTCTGCGCCTGTGCMNNMNNMNNCACCGCCAAAGTTTGGGCACT (SEQ ID NO: 70 of WO2017100671 wherein N may be A, C, T, or G; herein SEQ ID NO: 1360), GTATTCCTTGGTTTTGAACCCAACCGGTCTGCGCCTGTGCCTTAAAMNNMNNMNNCAGTTTGGGCACTCTGGTGG (SEQ ID NO: 71 of WO2017100671 wherein N may be A, C, T, or G; herein SEQ ID NO: 1361), GTATTCCTTGGTTTTGAACCCAACCGGTCTGCGCCTGTGCCTTAAAAGGCACMNNMNNMNNTTGGGCACTCTGGTGGTTTGTG (SEQ ID NO: 72 of WO2017100671 wherein N may be A, C, T, or G; herein SEQ ID NO: 1362), ACTTTGGCGGTGCCTTTTAAG (SEQ ID NO: 74 of WO2017100671; herein SEQ ID NO: 1279), AGTGTGAGTAAGCCTTTTTTG (SEQ ID NO: 75 of WO2017100671; herein SEQ ID NO: 1280), TTTACGTTGACGACGCCTAAG (SEQ ID NO: 76 of WO2017100671; herein SEQ ID NO: 1281), TATACTTTGTCGCAGGGTTGG (SEQ ID NO: 77 of WO2017100671; herein SEQ ID NO: 1287), or CTTGCGAAGGAGCGGCTTTCG (SEQ ID NO: 79 of WO2017100671; herein SEQ ID NO: 1363).

In some embodiments, the AAV serotype may be, or may have a sequence as described in U.S. Pat. No. 9,624,274, the contents of which are herein incorporated by reference in their entirety, such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ ID NO: 188 of U.S. Pat. No. 9,624,274), AAV11 (SEQ ID NO: 189 of U.S. Pat. No. 9,624,274), bAAV (SEQ ID NO: 190 of U.S. Pat. No. 9,624,274), AAV5 (SEQ ID NO: 191 of U.S. Pat. No. 9,624,274), GPV (SEQ ID NO: 192 of U.S. Pat. No. 9,624,274; herein SEQ ID NO: 879), B19 (SEQ ID NO: 193 of U.S. Pat. No. 9,624,274; herein SEQ ID NO: 880), MVM (SEQ ID NO: 194 of U.S. Pat. No. 9,624,274; herein SEQ ID NO: 881), FPV (SEQ ID NO: 195 of U.S. Pat. No. 9,624,274; herein SEQ ID NO: 882), CPV (SEQ ID NO: 196 of U.S. Pat. No. 9,624,274; herein SEQ ID NO: 883) or variants thereof. Further, any of the structural protein inserts described in U.S. Pat. No. 9,624,274, may be inserted into, but not limited to, I-453 and I-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 50 of U.S. Pat. No. 9,624,274; herein SEQ ID NO: 1364), EFCINHRGYWVCGD (SEQ ID NO:55 of U.S. Pat. No. 9,624,274; herein SEQ ID NO: 1365), EDGQVMDVDLS (SEQ ID NO: 85 of U.S. Pat. No. 9,624,274; herein SEQ ID NO: 1366), EKQRNGTLT (SEQ ID NO: 86 of U.S. Pat. No. 9,624,274; herein SEQ ID NO: 1367), TYQCRVTHPHLPRALMR (SEQ ID NO: 87 of U.S. Pat. No. 9,624,274; herein SEQ ID NO: 1368), RHSTTQPRKTKGSG (SEQ ID NO: 88 of U.S. Pat. No. 9,624,274; herein SEQ ID NO: 1369), DSNPRGVSAYLSR (SEQ ID NO: 89 of U.S. Pat. No. 9,624,274; herein SEQ ID NO: 1370), TITCLWDLAPSK (SEQ ID NO: 90 of U.S. Pat. No. 9,624,274; herein SEQ ID NO: 1371), KTKGSGFFVF (SEQ ID NO: 91 of U.S. Pat. No. 9,624,274; herein SEQ ID NO: 1372), THPHLPRALMRS (SEQ ID NO: 92 of U.S. Pat. No. 9,624,274; herein SEQ ID NO: 1373), GETYQCRVTHPHLPRALMRSTTK (SEQ ID NO: 93 of U.S. Pat. No. 9,624,274; herein SEQ ID NO: 1374), LPRALMRS (SEQ ID NO: 94 of U.S. Pat. No. 9,624,274; herein SEQ ID NO: 1375), INHRGYWV (SEQ ID NO: 95 of U.S. Pat. No. 9,624,274; herein SEQ ID NO: 1376), CDAGSVRTNAPD (SEQ ID NO: 60 of U.S. Pat. No. 9,624,274; herein SEQ ID NO: 1377), AKAVSNLTESRSESLQS (SEQ ID NO: 96 of U.S. Pat. No. 9,624,274; herein SEQ ID NO: 1378), SLTGDEFKKVLET (SEQ ID NO: 97 of U.S. Pat. No. 9,624,274; herein SEQ ID NO: 1379), REAVAYRFEED (SEQ ID NO: 98 of U.S. Pat. No. 9,624,274; herein SEQ ID NO: 1380), INPEIITLDG (SEQ ID NO: 99 of U.S. Pat. No. 9,624,274; herein SEQ ID NO: 1381), DISVTGAPVITATYL (SEQ ID NO: 100 of U.S. Pat. No. 9,624,274: herein SEQ ID NO: 1382), DISVTGAPVITA (SEQ ID NO: 101 of U.S. Pat. No. 9,624,274; herein SEQ ID NO: 1383), PKTVSNLTESSSESVQS (SEQ ID NO: 102 of U.S. Pat. No. 9,624,274; herein SEQ ID NO: 1384), SLMGDEFKAVLET (SEQ ID NO: 103 of U.S. Pat. No. 9,624,274; herein SEQ ID NO: 1385), QHSVAYTFEED (SEQ ID NO: 104 of U.S. Pat. No. 9,624,274; herein SEQ ID NO: 1386), INPEIITRDG (SEQ ID NO: 105 of U.S. Pat. No. 9,624,274; herein SEQ ID NO: 1387), DISLTGDPVITASYL (SEQ ID NO: 106 of U.S. Pat. No. 9,624,274; herein SEQ ID NO: 1388), DISLTGDPVITA (SEQ ID NO: 107 of U.S. Pat. No. 9,624,274; herein SEQ ID NO: 1389), DQSIDFEIDSA (SEQ ID NO: 108 of U.S. Pat. No. 9,624,274; herein SEQ ID NO: 1390), KNVSEDLPLPTFSPTLLGDS (SEQ ID NO: 109 of U.S. Pat. No. 9,624,274; herein SEQ ID NO: 1391), KNVSEDLPLPT (SEQ ID NO: 110 of U.S. Pat. No. 9,624,274; herein SEQ ID NO: 1392), CDSGRVRTDAPD (SEQ ID NO: 111 of U.S. Pat. No. 9,624,274; herein SEQ ID NO: 1393), FPEHLLVDFLQSLS (SEQ ID NO: 112 of U.S. Pat. No. 9,624,274; herein SEQ ID NO: 1394), DAEFRHDSG (SEQ ID NO: 65 of U.S. Pat. No. 9,624,274; herein SEQ ID NO: 1395), HYAAAQWDFGNTMCQL (SEQ ID NO: 113 of U.S. Pat. No. 9,624,274; herein SEQ ID NO: 1396), YAAQWDFGNTMCQ (SEQ ID NO: 114 of U.S. Pat. No. 9,624,274; herein SEQ ID NO: 1397), RSQKEGLHYT (SEQ ID NO: 115 of U.S. Pat. No. 9,624,274; herein SEQ ID NO: 1398), SSRTPSDKPVAHWANPQAE (SEQ ID NO: 116 of U.S. Pat. No. 9,624,274; herein SEQ ID NO: 1399), SRTPSDKPVAMWANP (SEQ ID NO: 117 of U.S. Pat. No. 9,624,274; herein SEQ ID NO: 1400), SSRTPSDKP (SEQ ID NO: 118 of U.S. Pat. No. 9,624,274; herein SEQ ID NO: 1401), NADGNVDYHMNSVP (SEQ ID NO: 119 of U.S. Pat. No. 9,624,274; herein SEQ ID NO: 1402), DGNVDYHMNSV (SEQ ID NO: 120 of U.S. Pat. No. 9,624,274; herein SEQ ID NO: 1403), RSFKEFLQSSLRALRQ (SEQ ID NO: 121 of U.S. Pat. No. 9,624,274; herein SEQ ID NO: 1404); FKEFLQSSLRA (SEQ ID NO: 122 of U.S. Pat. No. 9,624,274; herein SEQ ID NO: 1405), or QMWAPQWGPD (SEQ ID NO: 123 of U.S. Pat. No. 9,624,274; herein SEQ ID NO: 1406).

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not be limited to, the amino acid sequence RGNRQA (SEQ ID NO: 3 of U.S. Pat. No. 9,475,845; herein SEQ ID NO: 1407), SSSTDP (SEQ ID NO: 4 of U.S. Pat. No. 9,475,845; herein SEQ ID NO: 1408), SSNTAP (SEQ ID NO: 5 of U.S. Pat. No. 9,475,845; herein SEQ ID NO: 1409), SNSNLP (SEQ ID NO: 6 of U.S. Pat. No. 9,475,845; herein SEQ ID NO: 1410), SSTTAP (SEQ ID NO: 7 of U.S. Pat. No. 9,475,845; herein SEQ ID NO: 1411), AANTAA (SEQ ID NO: 8 of U.S. Pat. No. 9,475,845; herein SEQ ID NO: 1412), QQNTAP (SEQ ID NO: 9 of U.S. Pat. No. 9,475,845; herein SEQ ID NO: 1413), SAQAQA (SEQ ID NO: 10 of U.S. Pat. No. 9,475,845; herein SEQ ID NO: 1414), QANTGP (SEQ ID NO: 11 of U.S. Pat. No. 9,475,845; herein SEQ ID NO: 1415), NATTAP (SEQ ID NO: 12 of U.S. Pat. No. 9,475,845; herein SEQ ID NO: 1416), SSTAGP (SEQ ID NO: 13 and 20 of U.S. Pat. No. 9,475,845; herein SEQ ID NO: 1417), QQNTAA (SEQ ID NO: 14 of U.S. Pat. No. 9,475,845; herein SEQ ID NO: 1418), PSTAGP (SEQ ID NO: 15 of U.S. Pat. No. 9,475,845; herein SEQ ID NO: 1419), NQNTAP (SEQ ID NO: 16 of U.S. Pat. No. 9,475,845; herein SEQ ID NO: 1420), QAANAP (SEQ ID NO: 17 of U.S. Pat. No. 9,475,845; herein SEQ ID NO: 1421), SIVGLP (SEQ ID NO: 18 of U.S. Pat. No. 9,475,845; herein SEQ ID NO: 1422), AASTAA (SEQ ID NO: 19, and 27 of U.S. Pat. No. 9,475,845; herein SEQ ID NO: 1423), SQNTTA (SEQ ID NO: 21 of U.S. Pat. No. 9,475,845; herein SEQ ID NO: 1424), QQDTAP (SEQ ID NO: 22 of U.S. Pat. No. 9,475,845; herein SEQ ID NO: 1425), QTNTGP (SEQ ID NO: 23 of U.S. Pat. No. 9,475,845; herein SEQ ID NO: 1426), QTNGAP (SEQ ID NO: 24 of U.S. Pat. No. 9,475,845; herein SEQ ID NO: 1427), QQNAAP (SEQ ID NO: 25 of U.S. Pat. No. 9,475,845; herein SEQ ID NO: 1428), or AANTQA (SEQ ID NO: 26 of U.S. Pat. No. 9,475,845; herein SEQ ID NO: 1429). In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences, NGRAHA (SEQ ID NO: 38 of U.S. Pat. No. 9,475,845; herein SEQ ID NO: 1430), QPEHSST (SEQ ID NO: 39 and 50 of U.S. Pat. No. 9,475,845; herein SEQ ID NO: 1431), VNTANST (SEQ ID NO: 40 of U.S. Pat. No. 9,475,845; herein SEQ ID NO: 1432), HGPMQKS (SEQ ID NO: 41 of U.S. Pat. No. 9,475,845; herein SEQ ID NO: 1433), PHKPPLA (SEQ ID NO: 42 of U.S. Pat. No. 9,475,845; herein SEQ ID NO: 1434), IKNNEMW (SEQ ID NO: 43 of U.S. Pat. No. 9,475,845; herein SEQ ID NO: 1435), RNLDTPM (SEQ ID NO: 44 of U.S. Pat. No. 9,475,845; herein SEQ ID NO: 1436), VDSHRQS (SEQ ID NO: 45 of U.S. Pat. No. 9,475,845; herein SEQ ID NO: 1437), YDSKTKT (SEQ ID NO: 46 of U.S. Pat. No. 9,475,845; herein SEQ ID NO: 1438), SQLPHQK (SEQ ID NO: 47 of U.S. Pat. No. 9,475,845; herein SEQ ID NO: 1439), STMQQNT (SEQ ID NO: 48 of U.S. Pat. No. 9,475,845; herein SEQ ID NO: 1440), TERYMTQ (SEQ ID NO: 49 of U.S. Pat. No. 9,475,845; herein SEQ ID NO: 1441), DASLSTS (SEQ ID NO: 51 of U.S. Pat. No. 9,475,845; herein SEQ ID NO: 1442), DLPNKKT (SEQ ID NO: 52 of U.S. Pat. No. 9,475,845; herein SEQ ID NO: 1443), DLTAARL (SEQ ID NO: 53 of U.S. Pat. No. 9,475,845; herein SEQ ID NO: 1444), EPHQFNY (SEQ ID NO: 54 of U.S. Pat. No. 9,475,845; herein SEQ ID NO: 1445), EPQSNHT (SEQ ID NO: 55 of U.S. Pat. No. 9,475,845; herein SEQ ID NO: 1446), MSSWPSQ (SEQ ID NO: 56 of U.S. Pat. No. 9,475,845; herein SEQ ID NO: 1447), NPKHNAT (SEQ ID NO: 57 of U.S. Pat. No. 9,475,845; herein SEQ ID NO: 1448), PDGMRTT (SEQ ID NO: 58 of U.S. Pat. No. 9,475,845; herein SEQ ID NO: 1449), PNNNKTT (SEQ ID NO: 59 of U.S. Pat. No. 9,475,845; herein SEQ ID NO: 1450), QSTTHDS (SEQ ID NO: 60 of U.S. Pat. No. 9,475,845; herein SEQ ID NO: 1451), TGSKQKQ (SEQ ID NO: 61 of U.S. Pat. No. 9,475,845; herein SEQ ID NO: 1452), SLKHQAL (SEQ ID NO: 62 of U.S. Pat. No. 9,475,845; herein SEQ ID NO: 1453), SPIDGEQ (SEQ ID NO: 63 of U.S. Pat. No. 9,475,845; herein SEQ ID NO: 1454), WIFPWIQL (SEQ ID NO: 64 and 112 of U.S. Pat. No. 9,475,845; herein SEQ ID NO: 1455), CDCRGDCFC (SEQ ID NO: 65 of U.S. Pat. No. 9,475,845; herein SEQ ID NO: 1456), CNGRC (SEQ ID NO: 66 of U.S. Pat. No. 9,475,845; herein SEQ ID NO: 1457), CPRECES (SEQ ID NO: 67 of U.S. Pat. No. 9,475,845; herein SEQ ID NO: 1458), CTTHWGFTLC (SEQ ID NO: 68 and 123 of U.S. Pat. No. 9,475,845; herein SEQ ID NO: 1459), CGRRAGGSC (SEQ ID NO: 69 of U.S. Pat. No. 9,475,845; herein SEQ ID NO: 1460), CKGGRAKDC (SEQ ID NO: 70 of U.S. Pat. No. 9,475,845; herein SEQ ID NO: 1461), CVPELGHEC (SEQ ID NO: 71 and 115 of U.S. Pat. No. 9,475,845; herein SEQ ID NO: 1462), CRRETAWAK (SEQ ID NO: 72 of U.S. Pat. No. 9,475,845; herein SEQ ID NO: 1463), VSWFSHRYSPFAVS (SEQ ID NO: 73 of U.S. Pat. No. 9,475,845; herein SEQ ID NO: 1464), GYRDGYAGPILYN (SEQ ID NO: 74 of U.S. Pat. No. 9,475,845; herein SEQ ID NO: 1465), XXXYXXX (SEQ ID NO: 75 of U.S. Pat. No. 9,475,845; herein SEQ ID NO: 1466), YXNW (SEQ ID NO: 76 of U.S. Pat. No. 9,475,845; herein SEQ ID NO: 1467), RPLPPLP (SEQ ID NO: 77 of U.S. Pat. No. 9,475,845; herein SEQ ID NO: 1468), APPLPPR (SEQ ID NO: 78 of U.S. Pat. No. 9,475,845; herein SEQ ID NO: 1469), DVFYPYPYASGS (SEQ ID NO: 79 of U.S. Pat. No. 9,475,845; herein SEQ ID NO: 1470), MYWYPY (SEQ ID NO: 80 of U.S. Pat. No. 9,475,845; herein SEQ ID NO: 1471), DITWDQLWDLMK (SEQ ID NO: 81 of U.S. Pat. No. 9,475,845; herein SEQ ID NO: 1472), CWDDXWLC (SEQ ID NO: 82 of U.S. Pat. No. 9,475,845; herein SEQ ID NO: 1473), EWCEYLGGYLRCYA (SEQ ID NO: 83 of U.S. Pat. No. 9,475,845; herein SEQ ID NO: 1474), YXCXXGPXTWXCXP (SEQ ID NO: 84 of U.S. Pat. No. 9,475,845; herein SEQ ID NO: 1475), IEGPTLRQWLAARA (SEQ ID NO: 85 of U.S. Pat. No. 9,475,845; herein SEQ ID NO: 1476), LWXXX (SEQ ID NO: 86 of U.S. Pat. No. 9,475,845; herein SEQ ID NO: 1477), XFXXYLW (SEQ ID NO: 87 of U89475845; herein SEQ ID NO: 1478), SSIISHFRWGLCD (SEQ ID NO: 88 of U.S. Pat. No. 9,475,845; herein SEQ ID NO: 1479), MSRPACPPNDKYE (SEQ ID NO: 89 of U.S. Pat. No. 9,475,845; herein SEQ ID NO: 1480), CLRSGRGC (SEQ ID NO: 90 of U.S. Pat. No. 9,475,845; herein SEQ ID NO: 1481), CHWMFSPWC (SEQ ID NO: 91 of U.S. Pat. No. 9,475,845; herein SEQ ID NO: 1482), WXXF (SEQ ID NO: 92 of U.S. Pat. No. 9,475,845; herein SEQ ID NO: 1483), CSSRLDAC (SEQ ID NO: 93 of U.S. Pat. No. 9,475,845; herein SEQ ID NO: 1484), CLPVASC (SEQ ID NO: 94 of U.S. Pat. No. 9,475,845; herein SEQ ID NO: 1485), CGFECVRQCPERC (SEQ ID NO: 95 of U.S. Pat. No. 9,475,845; herein SEQ ID NO: 1486), CVALCREACGEGC (SEQ ID NO: 96 of U.S. Pat. No. 9,475,845; herein SEQ ID NO: 1487), SWCEPGWCR (SEQ ID NO: 97 of U.S. Pat. No. 9,475,845; herein SEQ ID NO: 1488), YSGKWGW (SEQ ID NO: 98 of U.S. Pat. No. 9,475,845; herein SEQ ID NO: 1489), GLSGGRS (SEQ ID NO: 99 of U.S. Pat. No. 9,475,845; herein SEQ ID NO: 1490), LMLPRAD (SEQ ID NO: 100 of U.S. Pat. No. 9,475,845; herein SEQ ID NO: 1491), CSCFRDVCC (SEQ ID NO: 101 of U.S. Pat. No. 9,475,845; herein SEQ ID NO: 1492), CRDVVSVIC (SEQ ID NO: 102 of U.S. Pat. No. 9,475,845; herein SEQ ID NO: 1493), MARSGL (SEQ ID NO: 103 of U.S. Pat. No. 9,475,845; herein SEQ ID NO: 1494), MARAKE (SEQ ID NO: 104 of U.S. Pat. No. 9,475,845; herein SEQ ID NO: 1495), MSRTMS (SEQ ID NO: 105 of U.S. Pat. No. 9,475,845; herein SEQ ID NO: 1496, KCCYSL (SEQ ID NO: 106 of U.S. Pat. No. 9,475,845; herein SEQ ID NO: 1497), MYWGDSHWLQYWYE (SEQ ID NO: 107 of U.S. Pat. No. 9,475,845; herein SEQ ID NO: 1498), MQLPLAT (SEQ ID NO: 108 of U.S. Pat. No. 9,475,845; herein SEQ ID NO: 1499), EWLS (SEQ ID NO: 109 of U.S. Pat. No. 9,475,845; herein SEQ ID NO: 1500), SNEW (SEQ ID NO: 110 of U.S. Pat. No. 9,475,845; herein SEQ ID NO: 1501), TNYL (SEQ ID NO: 111 of U.S. Pat. No. 9,475,845; herein SEQ ID NO: 1502), WDLAWMFRLPVG (SEQ ID NO: 113 of U.S. Pat. No. 9,475,845; herein SEQ ID NO: 1503), CTVALPGGYVRVC (SEQ ID NO: 114 of U.S. Pat. No. 9,475,845; herein SEQ ID NO: 1504), CVAYCIEHHCWTC (SEQ ID NO: 116 of U.S. Pat. No. 9,475,845; herein SEQ ID NO: 1505), CVFAHNYDYLVC (SEQ ID NO: 117 of U.S. Pat. No. 9,475,845; herein SEQ ID NO: 1506), CVFTSNYAFC (SEQ ID NO: 118 of U.S. Pat. No. 9,475,845; herein SEQ ID NO: 1507), VHSPNKK (SEQ ID NO: 119 of U.S. Pat. No. 9,475,845; herein SEQ ID NO: 1508), CRGDGWC (SEQ ID NO: 120 of U.S. Pat. No. 9,475,845; herein SEQ ID NO: 1509), XRGCDX (SEQ ID NO: 121 of U.S. Pat. No. 9,475,845; herein SEQ ID NO: 1510), PXXX (SEQ ID NO: 122 of U.S. Pat. No. 9,475,845; herein SEQ ID NO: 1511), SGKGPRQITAL (SEQ ID NO: 124 of U.S. Pat. No. 9,475,845; herein SEQ ID NO: 1512), AAAAAAAAAXXXXX (SEQ ID NO: 125 of U.S. Pat. No. 9,475,845; herein SEQ ID NO: 1513), VYMSPF (SEQ ID NO: 126 of U.S. Pat. No. 9,475,845; herein SEQ ID NO: 1514), ATWLPPR (SEQ ID NO: 127 of U.S. Pat. No. 9,475,845; herein SEQ ID NO: 1515), HTMYYHHYQHHL (SEQ ID NO: 128 of U.S. Pat. No. 9,475,845; herein SEQ ID NO: 1516), SEVGCRAGPLQWLCEKYFG (SEQ ID NO: 129 of U.S. Pat. No. 9,475,845; herein SEQ ID NO: 1517), CGLLPVGRPDRNVWRWLC (SEQ ID NO: 130 of U.S. Pat. No. 9,475,845; herein SEQ ID NO: 1518), CKGQCDRFKGLPWEC (SEQ ID NO: 131 of U.S. Pat. No. 9,475,845; herein SEQ ID NO: 1519), SGRSA (SEQ ID NO: 132 of U.S. Pat. No. 9,475,845; herein SEQ ID NO: 1520), WGFP (SEQ ID NO: 133 of U.S. Pat. No. 9,475,845; herein SEQ ID NO: 1521), AEPMPHSLNFSQYLWYT (SEQ ID NO: 134 of U.S. Pat. No. 9,475,845; herein SEQ ID NO: 1522), WAYXSP (SEQ ID NO: 135 of U.S. Pat. No. 9,475,845; herein SEQ ID NO: 1523), IELLQAR (SEQ ID NO: 136 of U.S. Pat. No. 9,475,845; herein SEQ ID NO: 1524), AYTKCSRQWRTCMTTH (SEQ ID NO: 137 of U.S. Pat. No. 9,475,845; herein SEQ ID NO: 1525), PQNSKIPGPTFLDPH (SEQ ID NO: 138 of U.S. Pat. No. 9,475,845; herein SEQ ID NO: 1526), SMEPALPDWWWKMFK (SEQ ID NO: 139 of U.S. Pat. No. 9,475,845; herein SEQ ID NO: 1527), ANTPCGPYTHDCPVKR (SEQ ID NO: 140 of U.S. Pat. No. 9,475,845; herein SEQ ID NO: 1528), TACHQHVRMVRP (SEQ ID NO: 141 of U.S. Pat. No. 9,475,845; herein SEQ ID NO: 1529), VPWMEPAYQRFL (SEQ ID NO: 142 of U.S. Pat. No. 9,475,845; herein SEQ ID NO: 1530), DPRATPGS (SEQ ID NO: 143 of U.S. Pat. No. 9,475,845; herein SEQ ID NO: 1531), FRPNRAQDYNTN (SEQ ID NO: 144 of U.S. Pat. No. 9,475,845; herein SEQ ID NO: 1532), CTKNSYLMC (SEQ ID NO: 145 of U.S. Pat. No. 9,475,845; herein SEQ ID NO: 1533), CXXTXXXGXGC (SEQ ID NO: 146 of U.S. Pat. No. 9,475,845; herein SEQ ID NO: 1534), CPIEDRPMC (SEQ ID NO: 147 of U.S. Pat. No. 9,475,845; herein SEQ ID NO: 1535), HEWSYLAPYPWF (SEQ ID NO: 148 of U.S. Pat. No. 9,475,845; herein SEQ ID NO: 1536), MCPKHPLGC (SEQ ID NO: 149 of U.S. Pat. No. 9,475,845; herein SEQ ID NO: 1537), RMWPSSTVNLSAGRR (SEQ ID NO: 150 of U.S. Pat. No. 9,475,845; herein SEQ ID NO: 1538), SAKTAVSQRVWLPSHRGGEP (SEQ ID NO: 151 of U.S. Pat. No. 9,475,845; herein SEQ ID NO: 1539), KSREHVNNSACPSKRITAAL (SEQ ID NO: 152 of U.S. Pat. No. 9,475,845; herein SEQ ID NO: 1540), EGFR (SEQ ID NO: 153 of U.S. Pat. No. 9,475,845; herein SEQ ID NO: 1541), AGLGVR (SEQ ID NO: 154 of U.S. Pat. No. 9,475,845; herein SEQ ID NO: 1542), GTRQGHTMRLGVSDG (SEQ ID NO: 155 of U.S. Pat. No. 9,475,845; herein SEQ ID NO: 1543), IAGLATPGWSHWLAL (SEQ ID NO: 156 of U.S. Pat. No. 9,475,845; herein SEQ ID NO: 1544), SMSIARL (SEQ ID NO: 157 of U.S. Pat. No. 9,475,845; herein SEQ ID NO: 1545), HTFEPGV (SEQ ID NO: 158 of U.S. Pat. No. 9,475,845; herein SEQ ID NO: 1546), NTSLKRISNKRIRRK (SEQ ID NO: 159 of U.S. Pat. No. 9,475,845; herein SEQ ID NO: 1547), LRIKRKRRKRKKTRK (SEQ ID NO: 160 of U.S. Pat. No. 9,475,845; herein SEQ ID NO: 1548), GGG, GFS, LWS, EGG, LLV, LSP, LBS, AGG, GRR, GGH and GTV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1549) or variants thereof, wherein the specific site is at least one site selected from sites R447, G453, S578, N587, N587+1, S662 of VP1 or fragment thereof.

Further, any of the mutated sequences described in US 20160369298, may be or may have, but not limited to, any of the following sequences SDSGASN (SEQ ID NO: 1 and SEQ ID NO: 231 of US20160369298; herein SEQ ID NO: 1550), SPSGASN (SEQ ID NO: 2 of US20160369298; herein SEQ ID NO: 1551), SHSGASN (SEQ ID NO: 3 of US20160369298; herein SEQ ID NO: 1552), SRSGASN (SEQ ID NO: 4 of US20160369298; herein SEQ ID NO: 1553), SKSGASN (SEQ ID NO: 5 of US20160369298; herein SEQ ID NO: 1554), SNSGASN (SEQ ID NO: 6 of US20160369298; herein SEQ ID NO: 1555), SGSGASN (SEQ ID NO: 7 of US20160369298; herein SEQ ID NO: 1556), SASGASN (SEQ ID NO: 8, 175, and 221 of US20160369298; herein SEQ ID NO: 1557), SESGTSN (SEQ ID NO: 9 of US20160369298; herein SEQ ID NO: 1558), STTGGSN (SEQ ID NO: 10 of US20160369298; herein SEQ ID NO: 1559), SSAGSTN (SEQ ID NO: 11 of US20160369298; herein SEQ ID NO: 1560), NNDSQA (SEQ ID NO: 12 of US20160369298; herein SEQ ID NO: 1561), NNRNQA (SEQ ID NO: 13 of US20160369298; herein SEQ ID NO: 1562), NNNKQA (SEQ ID NO: 14 of US20160369298; herein SEQ ID NO: 1563), NAKRQA (SEQ ID NO: 15 of US20160369298; herein SEQ ID NO: 1564), NDEHQA (SEQ ID NO: 16 of US20160369298; herein SEQ ID NO: 1565), NTSQKA (SEQ ID NO: 17 of US20160369298; herein SEQ ID NO: 1566), YYLSRTNTPSGTDTQSRLVFSQAGA (SEQ ID NO: 18 of US20160369298; herein SEQ ID NO: 1567), YYLSRTNTDSGTETQSGLDFSQAGA (SEQ ID NO:19 of US20160369298; herein SEQ ID NO:1568), YYLSRTNTESGTPTQSALEFSQAGA (SEQ ID NO: 20 of US20160369298; herein SEQ ID NO: 1569), YYLSRTNTHSGTHTQSPLHFSQAGA (SEQ ID NO: 21 of US20160369298; herein SEQ ID NO: 1570), YYLSRTNTSSGTITISHLIFSQAGA (SEQ ID NO: 22 of US20160369298; herein SEQ ID NO: 1571), YYLSRTNTRSGIMTKSSLMFSQAGA (SEQ ID NO: 23 of US20160369298; herein SEQ ID NO: 1572), YYLSRTNTKSGRKTLSNLSFSQAGA (SEQ ID NO: 24 of US20160369298; herein SEQ ID NO: 1573), YYLSRTNDGSGPVTPSKLRFSQRGA (SEQ ID NO: 25 of US20160369298; herein SEQ ID NO: 1574), YYLSRTNAASGHATHSDLKFSQPGA (SEQ ID NO: 26 of US20160369298; herein SEQ ID NO: 1575), YYLSRTNGQAGSLTMSELGFSQVGA (SEQ ID NO: 27 of US20160369298; herein SEQ ID NO: 1576), YYLSRTNSTGGNQTTSQLLFSQLSA (SEQ ID NO: 28 of US20160369298; herein SEQ ID NO: 1577), YFLSRTNNNTGLNTNSTLNFSQGRA (SEQ ID NO: 29 of US20160369298; herein SEQ ID NO: 1578), SKTGADNNNSEYSWTG (SEQ ID NO: 30 of US20160369298; herein SEQ ID NO: 1579), SKTDADNNNSEYSWTG (SEQ ID NO: 31 of US20160369298; herein SEQ ID NO: 1580), SKTEADNNNSEYSWTG (SEQ ID NO: 32 of US20160369298; herein SEQ ID NO: 1581), SKTPADNNNSEYSWTG (SEQ ID NO: 33 of US20160369298; herein SEQ ID NO: 1582), SKTHADNNNSEYSWTG (SEQ ID NO: 34 of US20160369298; herein SEQ ID NO: 1583), SKTQADNNNSEYSWTG (SEQ ID NO: 35 of US20160369298; herein SEQ ID NO: 1584), SKTIADNNNSEYSWTG (SEQ ID NO: 36 of US20160369298; herein SEQ ID NO: 1585), SKTMADNNNSEYSWTG (SEQ ID NO: 37 of US20160369298; herein SEQ ID NO: 1586), SKTRADNNNSEYSWTG (SEQ ID NO: 38 of US20160369298; herein SEQ ID NO: 1587), SKTNADNNNSEYSWTG (SEQ ID NO: 39 of US20160369298; herein SEQ ID NO: 1588), SKTVGRNNNSEYSWTG (SEQ ID NO: 40 of US20160369298; herein SEQ ID NO: 1589), SKTADRNNNSEYSWTG (SEQ ID NO: 41 of US20160369298; herein SEQ ID NO: 1590), SKKLSQNNNSKYSWQG (SEQ ID NO: 42 of US20160369298; herein SEQ ID NO: 1591), SKPTTGNNNSDYSWPG (SEQ ID NO: 43 of US20160369298; herein SEQ ID NO: 1592), STQKNENNNSNYSWPG (SEQ ID NO: 44 of US20160369298; herein SEQ ID NO: 1593), HKDDEGKF (SEQ ID NO: 45 of US20160369298; herein SEQ ID NO: 1594), HKDDNRKF (SEQ ID NO: 46 of US20160369298; herein SEQ ID NO: 1595), HKDDTNKF (SEQ ID NO: 47 of US20160369298; herein SEQ ID NO: 1596), HEDSDKNF (SEQ ID NO: 48 of US20160369298; herein SEQ ID NO: 1597), HRDGADSF (SEQ ID NO: 49 of US20160369298; herein SEQ ID NO: 1598), HGDNKSRF (SEQ ID NO: 50 of US20160369298; herein SEQ ID NO: 1599), KQGSEKTNVDFEEV (SEQ ID NO: 51 of US20160369298; herein SEQ ID NO: 1600), KQGSEKTNVDSEEV (SEQ ID NO: 52 of US20160369298; herein SEQ ID NO: 1601), KQGSEKTNVDVEEV (SEQ ID NO: 53 of US20160369298; herein SEQ ID NO: 1602), KQGSDKTNVDDAGV (SEQ ID NO: 54 of US20160369298; herein SEQ ID NO: 1603), KQGSSKTNVDPREV (SEQ ID NO: 55 of US20160369298; herein SEQ ID NO: 1604), KQGSRKTNVDHKQV (SEQ ID NO: 56 of US20160369298; herein SEQ ID NO: 1605), KQGSKGGNVDTNRV (SEQ ID NO: 57 of US20160369298; herein SEQ ID NO: 1606), KQGSGEANVDNGDV (SEQ ID NO: 58 of US20160369298; herein SEQ ID NO: 1607), KQDAAADNIDYDHV (SEQ ID NO: 59 of US20160369298; herein SEQ ID NO: 1608), KQSGTRSNAAASSV (SEQ ID NO: 60 of US20160369298; herein SEQ ID NO: 1609), KENTNTNDTELTNV (SEQ ID NO: 61 of US20160369298; herein SEQ ID NO: 1610), QRGNNVAATADVNT (SEQ ID NO: 62 of US20160369298; herein SEQ ID NO: 1611), QRGNNEAATADVNT (SEQ ID NO: 63 of US20160369298; herein SEQ ID NO: 1612), QRGNNPAATADVNT (SEQ ID NO: 64 of US20160369298; herein SEQ ID NO: 1613), QRGNNHAATADVNT (SEQ ID NO: 65 of US20160369298; herein SEQ ID NO: 1614), QEENNIAATPGVNT (SEQ ID NO: 66 of US20160369298; herein SEQ ID NO: 1615), QPPNNMAATHEVNT (SEQ ID NO: 67 of US20160369298; herein SEQ ID NO: 1616), QHHNNSAATTIVNT (SEQ ID NO: 68 of US20160369298; herein SEQ ID NO: 1617), QTTNNRAAFNMVET (SEQ ID NO: 69 of US20160369298; herein SEQ ID NO: 1618), QKKNNNAASKKVAT (SEQ ID NO: 70 of US20160369298; herein SEQ ID NO: 1619), QGGNNKAADDAVKT (SEQ ID NO: 71 of US20160369298; herein SEQ ID NO: 1620), QAAKGGAADDAVKT (SEQ ID NO: 72 of US20160369298; herein SEQ ID NO: 1621), QDDRAAAANESVDT (SEQ ID NO: 73 of US20160369298; herein SEQ ID NO: 1622), QQQHDDAAYQRVHT (SEQ ID NO: 74 of US20160369298; herein SEQ ID NO: 1623), QSSSSLAAVSTVQT (SEQ ID NO: 75 of US20160369298; herein SEQ ID NO: 1624), QNNQTTAAIRNVTT (SEQ ID NO: 76 of US20160369298; herein SEQ ID NO: 1625), NYNKKSDNVDFT (SEQ ID NO: 77 of US20160369298; herein SEQ ID NO: 1626), NYNKKSENVDFT (SEQ ID NO: 78 of US20160369298; herein SEQ ID NO: 1627), NYNKKSLNVDFT (SEQ ID NO: 79 of US20160369298; herein SEQ ID NO: 1628), NYNKKSPNVDFT (SEQ ID NO: 80 of US20160369298; herein SEQ ID NO: 1629), NYSKKSHCVDFT (SEQ ID NO: 81 of US20160369298; herein SEQ ID NO: 1630), NYRKTIYVDFT (SEQ ID NO: 82 of US20160369298; herein SEQ ID NO: 1631), NYKEKKDVHFT (SEQ ID NO: 83 of US20160369298; herein SEQ ID NO: 1632), NYGHRAIVQFT (SEQ ID NO: 84 of US20160369298; herein SEQ ID NO: 1633), NYANHQFVVCT (SEQ ID NO: 85 of US20160369298; herein SEQ ID NO: 1634), NYDDDPTGVLLT (SEQ ID NO: 86 of US20160369298; herein SEQ ID NO: 1635), NYDDPTGVLLT (SEQ ID NO: 87 of US20160369298; herein SEQ ID NO: 1636), NFEQQNSVEWT (SEQ ID NO: 88 of US20160369298; herein SEQ ID NO: 1637), SQSGASN (SEQ ID NO: 89 and SEQ ID NO: 241 of US20160369298; herein SEQ ID NO: 1638), NNGSQA (SEQ ID NO: 90 of US20160369298; herein SEQ ID NO: 1639), YYLSRTNTPSGTTTWSRLQFSQAGA (SEQ ID NO: 91 of US20160369298; herein SEQ ID NO: 1640), SKTSADNNNSEYSWTG (SEQ ID NO: 92 of US20160369298; herein SEQ ID NO: 1641), HKDDEEKF (SEQ ID NO: 93, 209, 214, 219, 224, 234, 239, and 244 of US20160369298; herein SEQ ID NO: 1642), KQGSEKTNVDIEEV (SEQ ID NO: 94 of US20160369298; herein SEQ ID NO: 1643), QRGNNQAATADVNT (SEQ ID NO: 95 of US20160369298; herein SEQ ID NO: 1644), NYNKKSVNVDFT (SEQ ID NO: 96 of US20160369298; herein SEQ ID NO: 1645), SQSGASNYNTPSGTTTQSRLQFSTSADNNNSEYSWTGATKYH (SEQ ID NO: 106 of US20160369298; herein SEQ ID NO: 1646), SASGASNFNSEGGSLTQSSLGFSTDGENNNSDFSWTGATKYH (SEQ ID NO: 107 of US20160369298; herein SEQ ID NO: 1647), SQSGASNYNTPSGTTTQSRLQFSTDGENNNSDFSWTGATKYH (SEQ ID NO: 108 of US20160369298; herein SEQ ID NO: 1648), SASGASNYNTPSGTTTQSRLQFSTSADNNNSEFSWPGATTYH (SEQ ID NO: 109 of US20160369298; herein SEQ ID NO: 1649), SQSGASNFNSEGGSLTQSSLGFSTDGENNNSDFSWTGATKYH (SEQ ID NO: 110 of US20160369298; herein SEQ ID NO: 1650), SASGASNYNTPSGSLTQSSLGFSTDGENNNSDFSWTGATKYH (SEQ ID NO: 111 of US20160369298; herein SEQ ID NO: 1651), SQSGASNYNTPSGTTTQSRLQFSTSADNNNSDFSWTGATKYH (SEQ ID NO: 112 of US20160369298; herein SEQ ID NO: 1652), SGAGASNFNSEGGSLTQSSLGFSTDGENNNSDFSWTGATKYH (SEQ ID NO: 113 of US20160369298; herein SEQ ID NO: 1653), SGAGASN (SEQ ID NO: 176 of US20160369298; herein SEQ ID NO: 1654), NSEGGSLTQSSLGFS (SEQ ID NO: 177, 185, 193 and 202 of US20160369298; herein SEQ ID NO: 1655), TDGENNNSDFS (SEQ ID NO: 178 of US20160369298; herein SEQ ID NO: 1656), SEFSWPGATT (SEQ ID NO: 179 of US20160369298; herein SEQ ID NO: 1657), TSADNNNSDFSWT (SEQ ID NO: 180 of US20160369298; herein SEQ ID NO: 1658), SQSGASNY (SEQ ID NO: 181,187, and 198 of US20160369298; herein SEQ ID NO: 1659), NTPSGTTTQSRLQFS (SEQ ID NO: 182,188,191, and 199 of US20160369298; herein SEQ ID NO: 1660), TSADNNNSEYSWTGATKYH (SEQ ID NO: 183 of US20160369298; herein SEQ ID NO: 1661), SASGASNF (SEQ ID NO: 184 of US20160369298; herein SEQ ID NO: 1662), TDGENNNSDFSWTGATKYH (SEQ ID NO: 186,189,194,197, and 203 of US20160369298; herein SEQ ID NO: 1663), SASGASNY (SEQ ID NO: 190 and SEQ ID NO: 195 of US20160369298: herein SEQ ID NO: 1664), TSADNNNSEFSWPGATTYH (SEQ ID NO: 192 of US20160369298; herein SEQ ID NO: 1665), NTPSGSLTQSSLGFS (SEQ ID NO: 196 of US20160369298; herein SEQ ID NO: 1666), TSADNNNSDFSWTGATKYH (SEQ ID NO: 200 of US20160369298; herein SEQ ID NO: 1667), SGAGASNF (SEQ ID NO: 201 of US20160369298; herein SEQ ID NO: 1668), CTCCAGVVSVVSMRSRVCVNSGCAGCTDHCVVSRNSGTCVMSACACAA (SEQ ID NO: 204 of US20160369298; herein SEQ ID NO: 1669), CTCCAGAGAGGCAACAGACAAGCAGCTACCGCAGATGTCAACACACAA (SEQ ID NO: 205 of US20160369298; herein SEQ ID NO: 1670), SAAGASN (SEQ ID NO: 206 of US20160369298; herein SEQ ID NO: 1671), YFLSRTNTESGSTTQSTLRFSQAG (SEQ ID NO: 207 of US20160369298; herein SEQ ID NO: 1672), SKTSADNNNSDFS (SEQ ID NO: 208, 228, and 253 of US20160369298; herein SEQ ID NO: 1673), KQGSEKTDVDIDKV (SEQ ID NO: 210 of US20160369298; herein SEQ ID NO: 1674), STAGASN (SEQ ID NO: 211 of US20160369298; herein SEQ ID NO: 1675), YFLSRTNTTSGIETQSTLRFSQAG (SEQ ID NO: 212 and SEQ ID NO: 247 of US20160369298; herein SEQ ID NO: 1676), SKTDGENNNSDFS (SEQ ID NO: 213 and SEQ ID NO: 248 of US20160369298; herein SEQ ID NO: 1677), KQGAAADDVEIDGV (SEQ ID NO: 215 and SEQ ID NO: 250 of US20160369298; herein SEQ ID NO: 1678), SEAGASN (SEQ ID NO: 216 of US20160369298; herein SEQ ID NO: 1679), YYLSRTNTPSGTTTQSRLQFSQAG (SEQ ID NO: 217, 232 and 242 of US20160369298; herein SEQ ID NO: 1680), SKTSADNNNSEYS (SEQ ID NO: 218, 233, 238, and 243 of US20160369298; herein SEQ ID NO: 1681), KQGSEKTNVDIEKV (SEQ ID NO: 220, 225 and 245 of US20160369298; herein SEQ ID NO: 1682), YFLSRTNDASGSDTKSTLLFSQAG (SEQ ID NO: 222 of US20160369298; herein SEQ ID NO: 1683), STTPSENNNSEYS (SEQ ID NO: 223 of US20160369298; herein SEQ ID NO: 1684), SAAGATN (SEQ ID NO: 226 and SEQ ID NO: 251 of US20160369298; herein SEQ ID NO: 1685), YFLSRTNGEAGSATLSELRFSQAG (SEQ ID NO: 227 of US20160369298; herein SEQ ID NO: 1686), HGDDADRF (SEQ ID NO: 229 and SEQ ID NO: 254 of US20160369298; herein SEQ ID NO: 1687), KQGAEKSDVEVDRV (SEQ ID NO: 230 and SEQ ID NO: 255 of US20160369298; herein SEQ ID NO: 1688), KQDSGGDNIDIDQV (SEQ ID NO: 235 of US20160369298; herein SEQ ID NO: 1689), SDAGASN (SEQ ID NO: 236 of US20160369298; herein SEQ ID NO: 1690), YFLSRTNTEGGHDTQSTLRFSQAG (SEQ ID NO: 237 of US20160369298; herein SEQ ID NO: 1691), KEDGGGSDVAIDEV (SEQ ID NO: 240 of US20160369298; herein SEQ ID NO: 1692), SNAGASN (SEQ ID NO: 246 of US20160369298; herein SEQ ID NO: 1693), and YFLSRTNGEAGSATLSELRFSQPG (SEQ ID NO: 252 of US20160369298; herein SEQ ID NO: 1694). Non-limiting examples of nucleotide sequences that may encode the amino acid mutated sites include the following, AGCWMDCAGGARSCASCAAC (SEQ ID NO: 97 of US20160369298; herein SEQ ID NO: 1695), AACRACRRSMRSMAGGCA (SEQ ID NO: 98 of US20160369298; herein SEQ ID NO: 1696), CACRRGGACRRCRMSRRSARSTTT (SEQ ID NO: 99 of US20160369298; herein SEQ ID NO: 1697), TATTTCTTGAGCAGAACAAACRVCVVSRSCGGAMNCVHSACGMHSTCAWSCTTVDSTTTTCTCAGSBCRGSGCG (SEQ ID NO: 100 of US20160369298; herein SEQ ID NO: 1698), TCAAMAMMAVNSRVCSRSAACAACAACAGTRASTTCTCGTGGMMAGGA (SEQ ID NO: 101 of US20160369298; herein SEQ ID NO: 1699), AAGSAARRCRSCRVSRVARVCRATRYCGMSNHCRVMVRSGTC (SEQ ID NO: 102 of US20160369298; herein SEQ ID NO: 1700), CAGVVSWSMRSRVCVNSGCAGCTDHCWSRNSGTCVMSACA (SEQ ID NO: 103 of US20160369298; herein SEQ ID NO: 1701), AACTWCRVSVASMVSVHSDDTGTGSWSTKSACT (SEQ ID NO: 104 of US20160369298; herein SEQ ID NO: 1702), TTGTTGAACATCACCACGTGACGCACGTTC (SEQ ID NO: 256 of US20160369298; herein SEQ ID NO: 1703), TCCCCGTGGTTCTACTACATAATGTGGCCG (SEQ ID NO: 257 of US20160369298; herein SEQ ID NO: 1704), TTCCACACTCCGTTTTGGATAATGTTGAAC (SEQ ID NO: 258 of US20160369298; herein SEQ ID NO: 1705), AGGGACATCCCCAGCTCCATGCTGTGGTCG (SEQ ID NO: 259 of US20160369298; herein SEQ ID NO: 1706), AGGGACAACCCCTCCGACTCGCCCTAATCC (SEQ ID NO: 260 of US20160369298; herein SEQ ID NO: 1707), TCCTAGTAGAAGACACCCTCTCACTGCCCG (SEQ ID NO: 261 of US20160369298; herein SEQ ID NO: 1708), AGTACCATGTACACCCACTCTCCCAGTGCC (SEQ ID NO: 262 of US20160369298; herein SEQ ID NO: 1709), ATATGGACGTTCATGCTGATCACCATACCG (SEQ ID NO: 263 of US20160369298; herein SEQ ID NO: 1710), AGCAGGAGCTCCTTGGCCTCAGCGTGCGAG (SEQ ID NO: 264 of US20160369298; herein SEQ ID NO: 1711), ACAAGCAGCTTCACTATGACAACCACTGAC (SEQ ID NO: 265 of US20160369298; herein SEQ ID NO: 1712), CAGCCTAGGAACTGGCTTCCTGGACCCTGTTACCGCCAGCAGAGAGTCTCAAMAMMAVNSRVCSRSAACAACAACAGTRASTTCTCC TGGMMAGGAGCTACCAAGTACCACCTCAATGGCAGAGACTCTCTGGTGAATCCCGGACCAGCTATGGCAAGCCACRRGGACRRCR MSRRSARSTTTTTTCCTCAGAGCGGGGTTCTCATCTTTGGGAAGSAARRCRSCRVSRVARVCRATRYCGMSNHCRVMVRSGTCATGAT TACAGACGAAGAGGAGATCTGGAC (SEQ ID NO: 266 of US20160369298; herein SEQ ID NO: 1713), TGGGACAATGGCGGTCGTCTCTCAGAGTTKTKKT (SEQ ID NO: 267 of US20160369298; herein SEQ ID NO: 1714), AGAGGACCKKTCCTCGATGGTTCATGGTGGAGTTA (SEQ ID NO: 268 of US20160369298; herein SEQ ID NO: 1715), CCACTTAGGGCCTGGTCGATACCGTTCGGTG (SEQ ID NO: 269 of US20160369298; herein SEQ ID NO: 1716), and TCTCGCCCCAAGAGTAGAAACCCTTCSTTYYG (SEQ ID NO: 270 of US20160369298; herein SEQ ID NO: 1717).

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID NO: 1718), or AAV9 (SEQ ID NO: 11 of WO2016134375; herein SEQ ID NO: 1719). In some embodiments, modifications, such as insertions are made in AAV2 proteins at P34-5, T138-A139, A139-P140, G453. T454, N587. R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ ID NO: 1720), or GETRAPL (SEQ ID NO: 4 of WO2016134375; herein SEQ ID NO: 1721).

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In some embodiments, the AAV serotype may be modified as described in the International Publication WO2017083722 the contents of which are herein incorporated by reference in their entirety. AAV serotypes may include, AAV (Y705+731F+T492V), AAV2 (Y444+500+730F+T491V), AAV3 (Y705+731F), AAV5, AAV 5 (Y436+693+719F), AAV6 (VP3 variant Y705F1Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids SPAKFA (SEQ ID NO: 24 of WO2017015102; herein SEQ ID NO: 1722) or NKDKLN (SEQ ID NO:2 of WO2017015102; herein SEQ ID NO: 1723). The epitope may be inserted in the region of amino acids 665 to 670 based on the numbering of the VP1 capsid of AAV8 (SEQ ID NO: 3 of WO2017015102) and/or residues 664 to 668 of AAV3B (SEQ ID NO: 3).

In some embodiments, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 261S, 263A, 264S, 265T, 266G, 272H, 385S, 386Q, 5472R, V473D, N500E 547S, 709A, 710N, 716D, 717N, 718N, 720L, 56T, Q457T, N458Q, K459S, T492S, K493A, S586R, S587G, S588N, T589R and/or 722T of AAV (SEQ ID NO: 1 of WO2017058892) in any combination, 244N, 246Q, 248R, 249E, 2501, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 5891 of AAV6 (SEQ ID NO:6 WO2017058892) in any combination, 457T, 459N, 496G, 499N, 500N, 589Q, 590N and/or 592A of AAV8 (SEQ ID NO: 8 WO2017058892) in any combination, 451I, 452N, 453G, 454S, 455G, 456Q, 457N and/or 458Q of AAV9 (SEQ ID NO: 9 WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156,157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

The effectiveness of AAV payload delivery may be affected by preexisting neutralizing antibodies, which present a significant challenge for vector effectiveness in therapeutic applications. In some embodiments, one or more of the mutations described by Jose et al. may be included in AAV described herein to circumvent the effects of preexisting neutralizing antibodies in a subject. In some embodiments, the AAV may be AAV5. In some embodiments, the AAV may include a mutation at positions 443, 444, 471, 481, 483, 484, 520, 576, 577, and/or 578 of VP3 as described in Jose et al. (J Virol. 2018 Dec. 10; 93(1):e01394-18; the contents of which are herein incorporated by reference in their entirety). As a non-limiting example, the mutation at position 443 of VP3 may be N443Q, or N443T. As a non-limiting example, the mutation at position 444 of VP3 may be T444V. As a non-limiting example, the mutation at position 471 of VP3 may be R471E. As a non-limiting example, the mutation at position 481 of VP3 may be V481T, V481P, or V481Y. As a non-limiting example, the mutation at position 483 of VP3 may be R483A, R483K, or R483Q. As a non-limiting example, the mutation at position 484 of VP3 may be A484S, A484Q or deletion of A484. As a non-limiting example, the mutation at position 520 of VP3 may be T520A, or T520R. As a non-limiting example, the mutation at position 576 of VP3 may be S576A, or S576Q. As a non-limiting example, the mutation at position 577 of VP3 may be T577A, or T577V. As a non-limiting example, the mutation at position 578 of VP3 may be T578A, or T578Q.

In some embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may include a peptide such as, but not limited to, PHP.B, PHP.B2, PHP.B3, PHP.A, PHP.S, G2A12, G2A15, G2, G2B4, and G2B5. In some embodiments, these AAV serotypes may be AAV9 (SEQ ID NO: 11 or 138) derivatives with a 7-amino acid insert between amino acids 588-589. Non-limiting examples of these 7-amino acid inserts include TLAVPFK (PHP.B; SEQ ID NO: 1262), SVSKPFL (PHP.B2; SEQ ID NO: 1270), FTLTTPK (PHP.B3; SEQ ID NO: 1271), YTLSQGW (PHP.A; SEQ ID NO: 1277), QAVRTSL (PHP.S; SEQ ID NO: 1321), LAKERLS (G2; SEQ ID NO: 1322), MNSTKNV (G2B4; SEQ ID NO: 1323), and/or VSGGHHS (G2B5; SEQ ID NO: 1324).

In some embodiments, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In some embodiments, the AAV serotype is a serotype comprising the AAVPHP.N (PHP.N) peptide, or a variant thereof.

In some embodiments the AAV serotype is a serotype comprising the AAVPHP.B (PHP.B) peptide, or a variant thereof.

In some embodiments, the AAV serotype is a serotype comprising the AAVPHP.A (PHP.A) peptide, or a variant thereof.

In some embodiments, the V serotype is a serotype comprising the PHP.S peptide, or a variant thereof.

In some embodiments, the V serotype is a serotype comprising the PHP.B2 peptide, or a variant thereof.

In some embodiments, the V serotype is a serotype comprising the PHP.B3 peptide, or a variant thereof.

In some embodiments, the V serotype is a serotype comprising the G2B4 peptide, or a variant thereof.

In some embodiments, the AAV serotype is a serotype comprising the G2B5 peptide, or a variant thereof.

In some embodiments, the V serotype is VOY101, or a variant thereof. In some embodiments, the VOY101 capsid comprises the amino acid sequence SEQ ID NO: 1. In some embodiments, the VOY101 amino acid sequence is encoded by a nucleotide sequence comprising SEQ ID NO: 2. In some embodiments, the VOY101 capsid comprises an amino acid sequence at least 70% identical to SEQ ID NO: 1, such as, 70%, 75%, 80%, 85%, 90%, 91%, 92%, 93%, 94%, 95%,96%,97%, 98%, 99%, or greater than 99%. In some embodiments, the VOY101 capsid comprises a nucleotide sequence at least 70% identical to SEQ ID NO: 2, such as, 70%, 75%, 80%, 85%, 90%, 91%, 92%, 93%, 94%, 95%, 96%, 97%, 98%, 99%, or greater than 99%.

In some embodiments, the V serotype is VOY201, or a variant thereof. In some embodiments, the VOY201 capsid comprises the amino acid sequence SEQ ID NO: 4534. In some embodiments, the VOY201 amino acid sequence is encoded by a nucleotide sequence comprising SEQ ID NO: 3. In some embodiments, the VOY201 capsid comprises an amino acid sequence at least 70% identical to SEQ ID NO: 4534, such as, 70%, 75%, 80%, 85%, 90%, 91%, 92%, 93%, 94%, 95%, 96%, 97%, 98%, 99%, or greater than 99%. In some embodiments, the VOY201 capsid comprises a nucleotide sequence at least 70% identical to SEQ ID NO: 3, such as, 70%, 75%, 80%, 85%, 90%, 91%, 92%, 93%, 94%, 95%, 96%, 97%, 98%, 99%, or greater than 99%.

In some embodiments, the AAV serotype is PHP.B, or a variant thereof. In some embodiments, the PHP.B capsid comprises the amino acid sequence SEQ ID NO: 5. In some embodiments, the PHP.B amino acid sequence is encoded by a nucleotide sequence comprising SEQ ID NO: 6. In some embodiments, the PHP.B capsid comprises an amino acid sequence at least 70% identical to SEQ ID NO: 5, such as, 70%, 75%,80%, 85%,90%,91%, 92%, 93%, 94%, 95%, 96%, 97%,98%,99%, or greater than 99%. In some embodiments, the PHP.B capsid comprises a nucleotide sequence at least 70% identical to SEQ ID NO: 6, such as, 70%, 75%, 80%, 85%, 90%, 91%, 92%, 93%, 94%, 95%, 96%, 97%, 98%, 99%, or greater than 99%.

In some embodiments, the AAV serotype is PHP.N, or a variant thereof. In some embodiments, the PHP.N capsid comprises the amino acid sequence SEQ ID NO: 4. In some embodiments, the PHP.N capsid comprises an amino acid sequence at least 70% identical to SEQ ID NO: 4, such as, 70%, 75%,80%, 85%, 90%, 91%, 92%, 93%,94%, 95%, 96%, 97%,98%, 99%, or greater than 99%.

In some embodiments the V serotype is AAV9, or a variant thereof. In some embodiments, the AAV9 capsid comprises the amino acid sequence SEQ ID NO: 138. In some embodiments, the AAV9 amino acid sequence is encoded by a nucleotide sequence comprising SEQ ID NO: 137. In some embodiments, the AAV9 capsid comprises an amino acid sequence at least 70% identical to SEQ ID NO: 138, such as, 70%, 75%,80%, 85%, 90%,91%,92%, 93%,94%,95%, 96%,97%,98%,99%, or greater than 99%. In some embodiments, the AAV9 capsid comprises a nucleotide sequence at least 70% identical to SEQ ID NO: 137, such as, 70%, 75%, 80%, 85%, 90%, 91%, 92%, 93%, 94%, 95%, 96%, 97%, 98%, 99%, or greater than 99%.

In some embodiments, the AAV serotype is AAV9 K449R, or a variant thereof. In some embodiments, the AAV9 K449R capsid comprises the amino acid sequence SEQ ID NO: 11. In some embodiments, the AAV9 K449R capsid comprises an amino acid sequence at least 70% identical to SEQ ID NO: 11, such as, 70%,75%,80%, 85%,90%, 91%,92%,93%, 94%, 95%,96%, 97%, 98%,99%, or greater than 99%.

In some embodiments, the AAV capsid allows for blood brain barrier penetration following intravenous administration. Non-limiting examples of such AAV capsids include AAV9, AAV9 K449R, VOY101, VOY201, or AAV capsids comprising a peptide insert such as, but not limited to, AAVPHP.N (PHP.N), AAVPHP.B (PHP.B), PHP.S, G2A3, G2B4, G2B5, G2A12, G2A15, PHP.B2, PHP.B3, or AAVPHP.A (PHP.A).

In some embodiments, the AAV capsid is suitable for intramuscular administration and/or transduction of muscle fibers. Non-limiting examples of such AAV capsids include AAV2, AAV3, AAV8 and variants thereof such as, but not limited to, AAV2 variants, AAV2/3 variants, AAV8 variants, and/or AAV2/3/8 variants.

In some embodiments, the AAV serotype is an AAV2 variant. As a non-limiting example, the AAV serotype is an AAV2 variant comprising SEQ ID NO: 11285 or a fragment or variant thereof. As a non-limiting example, the AAV serotype is at least 70% identical to SEQ ID NO: 11285, such as, 70%, 75%,80%,85%, 90%,91%,92%, 93%, 94%,95%,96%, 97%, 98%,99%, or greater than 99%.

In some embodiments, the AAV serotype is an AAV2/3 variant. As a non-limiting example, the AAV serotype is an AAV213 variant comprising SEQ ID NO: 11415 or a fragment or variant thereof. As a non-limiting example, the AAV serotype is an AAV2/3 variant which is at least 70% identical to SEQ ID NO: 11415, such as, 70%,75%,80%, 85%,90%,91%, 92%, 93%,94%, 95%, 96%, 97%,98%,99%, or greater than 99%. As a non-limiting example, the AAV serotype is an AAV2/3 variant comprising SEQ ID NO: 11477 or a fragment or variant thereof. As a non-limiting example, the AAV serotype is an AAV2/3 variant which is at least 70% identical to SEQ ID NO: 11477, such as, 70%, 75%, 80%, 85%, 90%, 91%, 92%, 93%, 94%, 95%, 96%, 97%, 98%, 99%, or greater than 99%.

In some embodiments, the AAV serotype may comprise a capsid amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%,86%, 87%, 88%, 89%, 90%, 91%, 92%, 93%, 94%, 95%, 96%, 97%, 98%, 99%, or 100% identity to any of those described herein.

In some embodiments, the AAV serotype may be encoded by a capsid nucleic acid sequence with 50%, 51%,52%, 53%, 54%, 55%, 56%, 57%, 58%, 59%, 60%,61%,62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described herein.

In some embodiments, the AAV serotype is selected for use due to its tropism for cells of the central nervous system. In some embodiments, the cells of the central nervous system are neurons. In another embodiment, the cells of the central nervous system are astrocytes.

In some embodiments, the AAV serotype is selected for use due to its tropism for cells of the muscle(s).

In some embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther. Methods. 2017 Oct. 28(5):255-267; Hwang, et al. N. Terminal Acetylation of Cellular Proteins Creates Specific Degradation Signals. Science, 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1-) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1-).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1), or a combination of VP3 (Met+/AA1+) and VP3 (Met−/AA1−); and can also include similar optional combinations of VP2 (Met+/AA1) and VP2 (Met−/AA1−).

### AAV Envelopes

In some embodiments, the present disclosure provides an AAV vector that may comprise an AAV particle surrounded by a lipid bilayer, wherein the lipid bilayer may comprise one or more functional molecules. As a non-limiting example, the functional molecule may be an immune-suppressing molecule. The lipid bilayer may be referred to herein as an envelope. The AAV vector or AAV particle surrounded by said lipid bilayer may be referred to herein as an enveloped AAV vector, or an enveloped AAV particle. In some embodiments, the enveloped AAV vector exhibits reduced immunogenicity compared to an AAV vector without an envelope. In some embodiments, the AAV particle may be partially surrounded by an envelope. In some embodiments, the AAV particle may be completely surrounded by an envelope.

The immunosuppressive molecules include but are not limited to molecules (e.g., proteins) that down-regulate immune function of a host by any mechanism, such as by stimulating or up-regulating immune inhibitors or by inhibiting or down-regulating immune stimulating molecules and/or activators, or by otherwise reducing the immunogenicity of the enveloped AAV vector compared to an enveloped vector without the immunosuppressive molecules. Non-limiting examples of immunosuppressive molecules include immune checkpoint receptors and ligands. Exemplary immune-suppressing molecules include, but are not limited to, cytotoxic T lymphocyte-associated antigen (CTLA4), B7-1, B7-2, programmed cell death protein 1 (PD-1), programmed death-ligand 1 (PD-L1), programmed death-ligand 2 (PD-L2), cluster of differentiation (CD28), V-domain Ig suppressor of T cell activation (VISTA), T-cell immunoglobin and mucin domain-3 (TIM-3), galectin-9 (GAL9), T-cell immunoreceptor with Ig and ITIM domains (TIGIT), CD155, (lymphocyte-activation gene 3 (LAG3), B and T lymphocyte associated (BTLA) and herpesvirus entry mediator (HVEM).

In some embodiments, the enveloped AAV vector may comprise AAV particle surrounded by an envelope, wherein the AAV particle comprises a heterologous transgene, and the envelope comprises a lipid bilayer and one or more immunosuppressive molecules. In some embodiments, the enveloped AAV may have reduced immunogenicity compared to an AAV vector without immunosuppressive molecules in the lipid bilayer. In some embodiments, the enveloped AAV vectors, compositions and methods thereof may be described in International Publication No. WO2019/140311, the contents of each of which are herein incorporated by reference in their entirety.

In some embodiments, the immunosuppressive molecules stimulate immune inhibitors. In some embodiments, the immunosuppressive molecules inhibit immune stimulating molecules. In some embodiments, the envelope comprises immunosuppressive molecules that stimulate immune inhibitors and immunosuppressive molecules that inhibit immune stimulating molecules. In some embodiments, the envelope may further comprise targeting molecules that target the AAV vector to one or more cell types. In some embodiments, the targeting molecule may be an antibody. Generally, targeting molecules that target different cell or tissue types can be used depending on the desired destination for the AAV vector. Non-limiting examples include one or more of liver, muscle, heart, brain (for example, neurons, glial cells, astrocytes, etc.), kidney, lung, pancreas, stomach, intestines, bone marrow, blood cells (for example, leukocytes, lymphocytes, erythrocytes), ovaries, uterus, testes, and stem cells of any type.

In some embodiments, the AAV particle comprises a viral capsid and a viral genome. In some embodiments, the viral genome comprises one or more heterologous transgene. In some embodiments, the AAV vector comprises a capsid from human AAV serotype AAV1, AAV2, AAV3, AAV4, AAV5, AAV6, AAV7, AAV8, AAV9, AAV10, AAV11 or AAV12. In some embodiments, the AAV vector comprises an AAV viral genome comprising inverted terminal repeat (ITR) sequences from human AAV serotype AAV1, AA V2, AAV3, AAV4, AAV5, AAV, AAV7, AAV8, AAV9, or AAV10.

In some embodiments, the AAV capsid and the AAV ITR are from the same serotype or from different serotypes. In some embodiments, the AAV viral particle comprises an AAV viral capsid and an AAV viral genome from the same serotype. In some embodiments, the AAV viral genome and the AAV capsid are of different serotypes. For example, the AAV viral capsid may be an AAV6 viral capsid and the AAV viral genome may be an AAV2 viral genome. In some embodiments, the AAV may be a self-complementary AAV (scAAV).

In some embodiments, the enveloped AAV vector as described herein can be used to deliver a transgene to a cell or a subject. In some embodiments, the enveloped AAV vector as described herein can be used to treat a disease or disorder in a subject. Non-limiting examples of diseases or disorders include myotubularin myopathy, spinal muscular atrophy, Leber's congenital amaurosis, hemophilia A, hemophilia B, choroideremia, Huntington's disease, Batten disease, Leber hereditary optic neuropathy, Omithine transcarbamylase (OTC) deficiency, Pompe disease, Fabry disease, citrullinemia type 1, phenylketonuria (PKU), adrenoleukodystrophy, sickle cell disease, Niemann-Pick disease and beta thalassemia.

In some embodiments, the disclosure provides a method of producing an enveloped AAV vector with reduced immunogenicity. The method may comprise culturing viral producer cells to generate enveloped AAV particles. In some embodiments the viral producer cells may comprise a nucleic acid encoding AAV rep and cap genes; a nucleic acid encoding an AAV viral genome comprising a transgene and at least one ITR; and a nucleic acid encoding AAV helper genes. In some embodiments, nucleic acid encoding AAV rep and cap genes and/or the AAV viral genome may be transiently introduced in the producer cell line. In some embodiments, nucleic acid encoding AAV rep and cap genes and/or the AAV viral genome may be stably maintained in the producer cell line. In some embodiments, nucleic acid encoding AAV rep and cap genes and/or the AAV viral genome may be stably integrated into the genome of the producer cell line. In some embodiments, the AAV genome comprises two AAV ITRs. For example, the viral genome may comprise a heterologous transgene flanked by AAV ITRs. In some embodiments, one or more AAV helper functions may be provided by one or more of a plasmid, an adenovirus, a nucleic acid stably integrated into the cell genome or a herpes simplex virus (HSV). In some embodiments, the AAV helper functions comprise one or more of adenovirus E1A function, adenovirus E1B function, adenovirus E2A function, adenovirus E4 function and adenovirus VA function. In some embodiments, one or more AAV helper functions may be stably integrated into the host cell genome and other AAV helper functions may be delivered transiently. For example, in some embodiments, the AAV enveloped vector is prepared in 293 cells expressing adenovirus E1A and E1B functions. The other helper functions may be delivered transiently; for example, by plasmid or by replication-deficient adenovirus. In some embodiments, the AAV helper functions comprise one or more of HSV UL5 function, HSV UL8 function, HSV UL52 function, and HSV UL29 function.

Generally, enveloped AAV vectors can be produced by co-transfecting plasmids or other expression vectors encoding the viral production genes (e.g., Rep/Cap and helper genes) and a plasmid or other construct comprising the AAV ITR and payload nucleic acid. Transfection can be accomplished in any manner, such as, but not limited to, by using calcium phosphate transfection, polyethyleneimine (PEI) transfection, or by using an HSV based production system as described by Booth et al., 2004 (see Booth et al. (2004) Gene Ther, 11(10):82937, the contents of which are herein incorporated by reference in their entirety). In some embodiments, the viral genes can include, but are not limited to, AAV2, 5, 6, 8, or 9 structural genes Rep and Cap, flanked by the AAV2 ITRs, and necessary helper virus genes as described by Ayuso et al., 2014 (see Ayuso et al. (2014) Hum Gene Ther, 25:977-987, the contents of which are herein incorporated by reference in their entirety). Production can be done in any suitable manner, such as, but not limited to, by using an adherent or suspension production system, with or without serum (see Ayuso et al. (2014) Hum Gene Ther, 25:977-987; Xiao et al. (1998), J Viral, 72(3): 2224-2232; Ryu et al. (2013) Mal Ther, Volume 21.B, the contents of which are herein incorporated by reference in their entirety). When the enveloped AAV vector includes a targeting moiety as described herein, the targeting moiety can be used as an affinity ligand to aid in isolation/purification. Other methods for producing enveloped AAV vectors are known and can be used, provided the producer cell is engineered to overexpress the desired immunosuppressive molecules.

### Viral Genome Component: Inverted Terminal Repeats (ITRs)

The AAV particles of the present disclosure comprise a viral genome with at least one ITR region and a payload region. In some embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes may be comprised of naturally occurring polynucleotide sequences or recombinantly derived polynucleotide sequences.

The ITRs may be derived from the same serotype as the capsid, selected from any of the serotypes listed in Table 1, or a derivative thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs.

Independently, each ITR may be about 100 to about 150 nucleotides in length. An ITR may be about 100.105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146.150 nucleotides in length. In some embodiments, the ITRs are 140-142 nucleotides in length. Non-limiting examples of ITR length are 102, 130, 140, 141, 142, 145 nucleotides in length, and those having at least 95% identity thereto.

In some embodiments, each ITR may be 141 nucleotides in length.

In some embodiments, each ITR may be 130 nucleotides in length.

In some embodiments, the AAV particles comprise two ITRs and one ITR is 141 nucleotides in length and the other ITR is 130 nucleotides in length.

### Viral Genome Component Promoters

In some embodiments, the payload region of the viral genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides in a target cell may require a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In some embodiments, the promoter is deemed to be efficient when it drives expression of the polypeptide(s) encoded in the payload region of the viral genome of the AAV particle.

In some embodiments, the promoter is a promoter deemed to be efficient when it drives expression in the cell being targeted.

In some embodiments, the promoter drives expression of the polypeptides (e.g., a functional antibody) for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1.5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6.12 months, 1-2 years, 1-5 years, 2-5 years, 3-6 years, 3-8 years, 4-4 years, or 5-10 years.

In some embodiments, the promoter drives expression of the polypeptides (e.g., a functional antibody) for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US20110212529, the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmoduin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300.400, 300-500, 300-600, 300.700, 300-800, 400-500, 400-600, 400.700, 400.800, 500-600, 500-700, 500-800, 600.700, 600-800, or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800. Each component may have a length between 200.300, 200.400, 200-500, 200.600, 200-700, 200-800, 300.400, 300-500, 300-600, 300-700, 300-800, 400.500, 400.600, 400.700, 400-800, 500-600, 500-700, 500-800, 600.700, 600-800 or 700-800. In some embodiments, the promoter is a combination of a 382 nucleotide CMV-enhancer sequence and a 260 nucleotide CBA-promoter sequence.

In some embodiments, the viral genome comprises a ubiquitous promoter. Non-limiting examples of ubiquitous promoters include CMV, CBA (including derivatives CAG, CB6, CBh, etc.), EF-1α, PGK, UBC, GUSB (hGBp), and UCOE (promoter of HNRPA2B1-CBX3).

Yu et al. (Molecular Pain 2011, 7:63; the contents of which are herein incorporated by reference in their entirety) evaluated the expression of eGFP under the CAG, EFIα, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are herein incorporated by reference in its entirety) evaluated the expression of eGFP in AAV8 with CMV and UBC promoters and AAV2 with the CMV promoter after injection in the motor cortex. Intranasal administration of a plasmid containing a UBC or EFIα promoter showed a sustained airway expression greater than the expression with the CMV promoter (See e.g., Gill et al., Gene Therapy 2001, Vol. 8, 1539-1546; the contents of which are herein incorporated by reference in their entirety). Husain et al. (Gene Therapy 2009; the contents of which are herein incorporated by reference in its entirety) evaluated an HOH construct with a hGUSB promoter, an HSV-1LAT promoter and an NSE promoter and found that the HOH construct showed weaker expression than NSE in mouse brain. Passini and Wolfe (J. Virol. 2001, 12382-12392, the contents of which are herein incorporated by reference in its entirety) evaluated the long-term effects of the HOH vector following an intraventricular injection in neonatal mice and found that there was sustained expression for at least 1 year. Low expression in all brain regions was found by Xu et al. (Gene Therapy 2001, 8, 1323-1332; the contents of which are herein incorporated by reference in their entirety) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920-nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus, and hypothalamus (See e.g., Drews et al. Identification of evolutionary conserved, functional noncoding elements in the promoter region of the sodium channel gene SCN8A, Mamm Genome (2007) 18:723-731; and Raymond et al. Expression of Alternatively Spliced Sodium Channel α-subunit genes, Journal of Biological Chemistry (2004) 279(44) 46234-46241; the contents of each of which are herein incorporated by reference in their entireties).

Any of promoters taught by the aforementioned Yu, Soderblom, Gill, Husain, Passini, Xu, Drews, or Raymond may be used in the present disclosures.

In some embodiments, the promoter is not cell specific.

In some embodiments, the promoter is a ubiquitin c (UBC) promoter. The UBC promoter may have a size of 300-350 nucleotides. As a non-limiting example, the UBC promoter is 332 nucleotides.

In some embodiments, the promoter is a β-glucuronidase (GUSB) promoter. The GUSB promoter may have a size of 350-400 nucleotides. As a non-limiting example, the GUSB promoter is 378 nucleotides.

In some embodiments, the promoter is a neurofilament light (NFL) promoter. The NFL promoter may have a size of 600-700 nucleotides. As a non-limiting example, the NFL promoter is 650 nucleotides.

In some embodiments, the promoter is a neurofilament heavy (NFH) promoter. The NFH promoter may have a size of 900-950 nucleotides. As a non-limiting example, the NFH promoter is 920 nucleotides.

In some embodiments, the promoter is a scn8a promoter. The scn8a promoter may have a size of 450-500 nucleotides. As a non-limiting example, the scn8a promoter is 470 nucleotides.

In some embodiments, the promoter is a phosphoglycerate kinase 1 (PGK) promoter.

In some embodiments, the promoter is a chicken β-actin (CBA) promoter, or a variant thereof.

In some embodiments, the promoter is a CB6 promoter.

In some embodiments, the promoter is a minimal CB promoter.

In some embodiments, the promoter is a cytomegalovirus (CMV) promoter.

In some embodiments, the promoter is a CAG promoter.

In some embodiments, the promoter is a GFAP promoter.

In some embodiments, the promoter is a synapsin promoter.

In some embodiments, the promoter is a liver or a skeletal muscle promoter. Non-limiting examples of liver promoters include human α-1-antitrypsin (hAAT) and thyroxine binding globulin (TBG). Non-limiting examples of skeletal muscle promoters include Desmin, MCK or synthetic C5-12.

In some embodiments, the promoter is an RNA pol III promoter. As a non-limiting example, the RNA pol III promoter is U6. As a non-limiting example, the RNA pol III promoter is H1.

In some embodiments, the viral genome comprises two promoters. As a non-limiting example, the promoters are an EF1α promoter and a CMV promoter.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

In some embodiments, the viral genome comprises an engineered promoter.

In another embodiment, the viral genome comprises a promoter from a naturally expressed protein.

### Viral Genome Component: Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/BIE, transferrin, alpha fetoprotein, erythropoietin, or Factor VIII) may be used in the viral genomes of the AAV particles to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

In some embodiments, the Kozak sequence is GAGGAGCCACC (SEQ ID NO: 13149).

In some embodiments, the Kozak sequence is GCCGCCACCATG (SEQ ID NO: 13563)

While not wishing to be bound by theory, wild-type 3′UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′UTR of the viral genome may include an oligo(dT) sequence for templated addition of a poly-A tail.

In some embodiments, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some embodiments, the viral genome may be engineered to include, alter or remove at least one miRNA binding site, sequence, or seed region.

Any UTR from any gene known in the art may be incorporated into the viral genome of the AAV particle. These UTRs, or portions thereof, may be placed in the same orientation as in the gene from which they were selected, or they may be altered in orientation or location. In some embodiments, the UTR used in the viral genome of the AAV particle may be inverted, shortened, lengthened, made with one or more other 5′UTRs or 3′UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTRs which is not a variant of a wild type UTR.

In some embodiments, the viral genome of the AAV particle comprises UTRs which have been selected from a family of transcripts whose proteins share a common function, structure, feature or property.

### Viral Genome Component: Polyadenylation Sequence

In some embodiments, the viral genome of the AAV particles of the present disclosure comprise at least one polyadenylation sequence. The viral genome of the AAV particle may comprise a polyadenylation sequence between the 3′ end of the payload coding sequence and the 5′ end of the 3′ITR.

In some embodiments, the polyadenylation sequence or “polyA sequence” may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides in length.

In some embodiments, the polyadenylation sequence is 50-100 nucleotides in length.

In some embodiments, the polyadenylation sequence is 50-150 nucleotides in length.

In some embodiments, the polyadenylation sequence is 50-160 nucleotides in length.

In some embodiments, the polyadenylation sequence is 50-200 nucleotides in length.

In some embodiments, the polyadenylation sequence is 60-100 nucleotides in length.

In some embodiments, the polyadenylation sequence is 60-150 nucleotides in length.

In some embodiments, the polyadenylation sequence is 60-160 nucleotides in length.

In some embodiments, the polyadenylation sequence is 60-200 nucleotides in length.

In some embodiments, the polyadenylation sequence is 70-100 nucleotides in length.

In some embodiments, the polyadenylation sequence is 70-150 nucleotides in length.

In some embodiments, the polyadenylation sequence is 70-160 nucleotides in length.

In some embodiments, the polyadenylation sequence is 70-200 nucleotides in length.

In some embodiments, the polyadenylation sequence is 80-100 nucleotides in length.

In some embodiments, the polyadenylation sequence is 80-150 nucleotides in length.

In some embodiments, the polyadenylation sequence is 80-160 nucleotides in length.

In some embodiments, the polyadenylation sequence is 80-200 nucleotides in length.

In some embodiments, the polyadenylation sequence is 90-100 nucleotides in length.

In some embodiments, the polyadenylation sequence is 90-150 nucleotides in length.

In some embodiments, the polyadenylation sequence is 90.160 nucleotides in length.

In some embodiments, the polyadenylation sequence is 90.200 nucleotides in length.

In some embodiments, the polyadenylation sequence is 127 nucleotides in length.

In some embodiments, the polyadenylation sequence is 477 nucleotides in length.

In some embodiments, the polyadenylation sequence is 552 nucleotides in length.

### Viral Genome Component: Linkers

Viral genomes may be engineered with one or more spacer or linker regions to separate coding or non-coding regions.

In some embodiments, the payload region of the AAV particle may optionally encode one or more linker sequences. In some cases, the linker may be a peptide linker that may be used to connect the polypeptides encoded by the payload region (i.e., light and heavy antibody chains during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome. Non-limiting examples of linkers that may be encoded by the payload region of an AAV particle viral genome are given in Table 2.

TABLE 2
Linkers

| | | | SEQ |
|---|---|---|---|
| Linker ID | Description | Length | ID NO |
| Linker1 | Furin | 12 | 1724 |
| Linker2 | Furin | 12 | 1725 |
| Linker3 | T2A | 54 | 1726 |
| Linker4 | F2A | 75 | 1727 |
| Linker5 | P2A | 66 | 1728 |
| Linker6 | SG4S (SEQ ID NO: 13142) | 18 | 1729 |
| Linker7 | (G4S)3 (SEQ ID NO: 13143) | 45 | 1730 |
| Linker8 | (G4S)5 (SEQ ID NO: 13144) | 75 | 1731 |
| Linker9 | IRES | 609 | 1732 |
| Linker10 | IRES-2 | 623 | 1733 |
| Linker11 | hIgG2 hinge | 54 | 1734 |
| Linker12 | hIgG3 hinge | 108 | 1735 |
| Linker13 | hIgG3-2 hinge | 153 | 1736 |
| Linker14 | hIgG3-3 hinge | 198 | 1737 |
| Linker15 | msiGG-1 hinge | 45 | 1738 |
| Linker16 | msiGG1 hinge | 18 | 1739 |
| Linker17 | HigG3 hinge | 198 | 2244 |
| Linker18 | G4S (SEQ ID NO: 13141) | 15 | 2245 |
| Linker19 | (G4S)2 (SEQ ID NO: 13145) | 30 | 2246 |
| Linker20 | (G4S)3 (SEQ ID NO: 13143) | 45 | 2247 |
| Linker21 | (G4S)4 (SEQ ID NO: 13146) | 60 | 2248 |
| Linker22 | (G4S)5 (SEQ ID NO: 13144) | 75 | 2249 |
| Linker23 | (G4S)5 (SEQ ID NO: 13144) | 75 | 2250 |
| Linker24 | (G4S)6 (SEQ ID NO: 13147) | 90 | 2251 |
| Linker25 | (G4S)8 (SEQ ID NO: 13148) | 120 | 2252 |
| Linker26 | (G4S)8 (SEQ ID NO: 13148) | 120 | 2253 |
| Linker27 | (G4S)4 (SEQ ID NO: 13146) | 60 | 2254 |
| Linker28 | (G4S)6 (SEQ ID NO: 13147) | 90 | 2259 |

Some payload regions encode linkers comprising furin cleavage sites. Furin is a calcium dependent serine endoprotease that cleaves proteins just downstream of a basic amino acid target sequence (Arg-X-(Arg/Lys)-Arg) (Thomas, G., 2002. Nature Reviews Molecular Cell Biology 3(10): 753-66; the contents of which are herein incorporated by reference in its entirety). Furin is enriched in the trans-golgi network where it is involved in processing cellular precursor proteins. Furin also plays a role in activating a number of pathogens. This activity can be taken advantage of for expression of polypeptides.

2A peptides are small “self-cleaving” peptides (18-22 amino acids) derived from viruses such as foot-and-mouth disease virus (F2A), porcine teschovirus-1 (P2A), Thoseaasigna virus (T2A), or equine rhinitis A virus (E2A). The 2A designation refers specifically to a region of picornavirus polyproteins that lead to a ribosomal skip at the glycyl-prolyl bond in the C-terminus of the 2A peptide (Kim, J. H. et al., 2011. PLoS One 6(4): e18556; the contents of which are herein incorporated by reference in its entirety). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

Internal ribosomal entry site (IRES) is a nucleotide sequence (>500 nucleotides) that allows for initiation of translation in the middle of an mRNA sequence (Kim, J. H. et al., 2011. PLoS One 6(4): e18556; the contents of which are herein incorporated by reference in its entirety). Use of an IRES sequence ensures co-expression of genes before and after the IRES, though the sequence following the IRES may be transcribed and translated at lower levels than the sequence preceding the IRES sequence.

In some embodiments, the payload region may encode one or more linkers comprising cathepsin, matrix metalloproteinases or legumain cleavage sites. Such linkers are described e.g. by Cizeau and Macdonald in International Publication No. WO2008052322, the contents of which are herein incorporated in their entirety. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In some embodiments, payload regions may encode linkers that are not cleaved. Such linkers may include a simple amino acid sequence, such as a glycine rich sequence. In some cases, linkers may comprise flexible peptide linkers comprising glycine and serine residues. The linker may comprise flexible peptide linkers of different lengths, e.g. nxG4S, where n=1-10 (SEQ ID NO: 13150), and the length of the encoded linker varies between 5 and 50 amino acids. In a non-limiting example, the linker may be 5xG4S (SEQ ID NO: 13144). These flexible linkers are small and without side chains so they tend not to influence secondary protein structure while providing a flexible linker between antibody segments (George, R A, et al., 2002. Protein Engineering 15(11): 871-9; Huston, J. S. et al, 1988. PNAS 85:5879-83; and Shan, D. et al., 1999. Journal of Immunology. 162(11):6589-95; the contents of each of which are herein incorporated by reference in their entirety). Furthermore, the polarity of the serine residues improves solubility and prevents aggregation problems.

In some embodiments, payload regions may encode small and unbranched serine-rich peptide linkers, such as those described by Huston et al. In U.S. Pat. No. 5,525,491, the contents of which are herein incorporated in their entirety. Polypeptides encoded by the payload region, linked by serine-rich linkers, have increased solubility.

In some embodiments, payload regions may encode artificial linkers, such as those described by Whitlow and Filpula in U.S. Pat. No. 5,856,456 and Ladner et al. in U.S. Pat. No. 4,946,778, the contents of each of which are herein incorporated by their entirety.

In some embodiments, the payload region encodes at least one G4S3 linker (“G43” disclosed as SEQ ID NO: 13143).

In some embodiments, the payload region encodes at least one G4S linker (“G4S” disclosed as SEQ ID NO: 13141).

In some embodiments, the payload region encodes at least one furin site.

In some embodiments, the payload region encodes at least one T2A linker.

In some embodiments, the payload region encodes at least one F2A linker.

In some embodiments, the payload region encodes at least one P2A linker.

In some embodiments, the payload region encodes at least one IRES sequence.

In some embodiments, the payload region encodes at least one G4S5 linker (“G4S5” disclosed as SEQ ID NO: 13144).

In some embodiments, the payload region encodes at least one furin and one 2A linker. As non-limiting examples, the payload region may comprise furin and T2A linkers or furin and F2A linkers.

In some embodiments, the payload region encodes at least one hinge region. As a non-limiting example, the hinge is an IgG hinge.

In some embodiments, the linker region may be 1.50, 1-100, 50-100, 50-150, 100-150, 100-200, 150-200, 150-250, 200-250, 200-300, 250-300, 250-350, 300-350, 300-400, 350-400, 350-450, 400-450, 400-500, 450-500, 450-550, 500-550, 500-600, 550-600, 550-650, or 600-650 nucleotides in length. The linker region may have a length of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 115, 120, 125, 130, 135, 140, 145, 150, 151, 152, 153, 154, 155, 156, 157, 158, 159, 160, 165, 170, 171, 172, 173, 174, 175, 176, 177, 178, 179, 180, 185, 190, 191, 192, 193, 194, 195, 196, 197, 198,199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 640, 650 or greater than 650. In some embodiments, the linker region may be 12 nucleotides in length. In some embodiments, the linker region may be 15 nucleotides in length. In some embodiments, the linker region may be 18 nucleotides in length. In some embodiments, the linker region may be 30 nucleotides in length. In some embodiments, the linker region may be 45 nucleotides in length. In some embodiments, the linker region may be 54 nucleotides in length. In some embodiments, the linker region may be 60 nucleotides in length. In some embodiments, the linker region may be 66 nucleotides in length. In some embodiments, the linker region may be 75 nucleotides in length. In some embodiments, the linker region may be 78 nucleotides in length. In some embodiments, the linker region may be 87 nucleotides in length. In some embodiments, the linker region may be 108 nucleotides in length. In some embodiments, the linker region may be 120 nucleotides in length. In some embodiments, the linker region may be 153 nucleotides in length. In some embodiments, the linker region may be 198 nucleotides in length. In some embodiments, the linker region may be 609 nucleotides in length. In some embodiments, the linker region may be 623 nucleotides in length.

### Viral Genome Component: Introns

In some embodiments, the payload region comprises at least one element to enhance the expression such as one or more introns or portions thereof. Non-limiting examples of introns include, MVM (67.97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some embodiments, the intron or intron portion may be 1-100, 100-500, 500-1000, or 1000-1500 nucleotides in length. The intron may have a length of 10, 15, 20, 30, 40, 50, 60, 70, 80, 90, 100, 110, 120, 130, 140, 150, 160, 170, 171, 172, 173, 174, 175, 176, 177, 178, 179, 180, 190,200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or greater than 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500. In some embodiments, the intron may be 15 nucleotides in length. In some embodiments, the intron may be 32 nucleotides in length. In some embodiments, the intron may be 41 nucleotides in length. In some embodiments, the intron may be 53 nucleotides in length. In some embodiments, the intron may be 54 nucleotides in length. In some embodiments, the intron may be 59 nucleotides in length. In some embodiments, the intron may be 73 nucleotides in length. In some embodiments, the intron may be 102 nucleotides in length. In some embodiments, the intron may be 134 nucleotides in length. In some embodiments, the intron may be 168 nucleotides in length. In some embodiments, the intron may be 172 nucleotides in length. In some embodiments, the intron may be 292 nucleotides in length. In some embodiments, the intron may be 347 nucleotides in length. In some embodiments, the intron may be 387 nucleotides in length. In some embodiments, the intron may be 491 nucleotides in length. In some embodiments, the intron may be 566 nucleotides in length. In some embodiments, the intron may be 1074 nucleotides in length.

Any, or all components of a viral genome may be modified or optimized to improve expression or targeting of the payload. Such components include, but are not limited to, intron, signal peptide sequences, antibody heavy chain and/or light chain 5 to 3′ order, antibody heavy chain and/or light chain codons, linkers, cleavage sites, polyadenylation sequences, stuffer sequences, other regulatory sequences, and/or the backbone of the ITR to ITR sequence.

### Payloads

The AAV particles of the present disclosure comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Payloads of the present disclosure typically encode polypeptides (e.g., antibodies or antibody-based compositions) or fragments or variants thereof.

The payload region may be constructed in such a way as to reflect a region similar to or mirroring the natural organization of an mRNA.

The payload region may comprise a combination of coding and non-coding nucleic acid sequences.

In some embodiments, the AAV payload region may encode a coding or non-coding RNA.

In some embodiments, the AAV particle comprises a viral genome with a payload region comprising nucleic acid sequences encoding more than one polypeptide of interest (e.g., an antibody). In such an embodiment, a viral genome encoding more than one polypeptide may be replicated and packaged into a viral particle. A target cell transduced with a viral particle comprising more than one polypeptide may express each of the polypeptides in a single cell.

In some embodiments, an AAV particle comprises a viral genome with a payload region comprising a nucleic acid sequence encoding a heavy chain and alight chain of an antibody, or fragments thereof. The heavy chain and light chain are expressed and assembled to form the antibody which is secreted.

In some embodiments, the payload region may comprise at least one inverted terminal repeat (ITR), a promoter region, an intron region, and a coding region. In some embodiments, the coding region comprises a heavy chain region and/or a light chain region of an antibody, or a fragment thereof, and any two components may be separated by a linker region.

In some embodiments, the coding region may comprise a payload region with a heavy chain and light chain sequence separated by a linker and/or a cleavage site. In some embodiments, the heavy and light chain sequence is separated by an IRES sequence. In some embodiments, the heavy and light chain sequence is separated by a foot and mouth virus sequence. In some embodiments, the heavy and light chain sequence is separated by a foot and mouth virus sequence and a furin cleavage site. In some embodiments, the heavy and light chain sequence is separated by a porcine teschovirus-1 virus sequence. In some embodiments, the heavy and light chain sequence is separated by a porcine teschovirus-1 virus and a furin cleavage site. In some embodiments, the heavy and light chain sequence is separated by a 5xG4S sequence (“5xG4S” disclosed as SEQ ID NO: 13144).

Where the AAV particle payload region encodes a polypeptide, the polypeptide may be a peptide or protein. A protein encoded by the AAV particle payload region may comprise an antibody, an antibody related composition, a secreted protein, an intracellular protein, an extracellular protein, and/or a membrane protein. The encoded proteins may be structural or functional. In addition to the antibodies or antibody-based composition, proteins encoded by the payload region may include, in combination, certain mammalian proteins involved in immune system regulation. The V viral genomes encoding polypeptides described herein may be useful in the fields of human disease, viruses, infections, veterinary applications and a variety of in vivo and in vitro settings.

In some embodiments, the AAV particles are useful in the field of medicine for the treatment, prophylaxis, palliation, or amelioration of neurological diseases and/or disorders.

In some embodiments, the AAV particle payload region may one or more include therapeutic modalities related to gene silencing or interference such as but not limited to, miRNA, siRNA, RNAi, shRNA, and/or pri-miRNA.

### Antibodies and Antibody-Based Compositions

Payload regions of the AAV particles may encode polypeptides that form one or more functional antibodies or antibody-based compositions. As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.).

As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Payload regions may encode polypeptides that form or function as any antibody, including antibodies that are known in the art and/or antibodies that are commercially available. The encoded antibodies may be therapeutic, diagnostic, or for research purposes. Further, polypeptides may include fragments of such antibodies or antibodies that have been developed to comprise one or more of such fragments (e.g., variable domains or complementarity determining regions (CDRs)).

In some embodiments, the viral genome of the AAV particles may comprise nucleic acids which have been engineered to enable expression of antibodies, antibody fragments, or components of any of those described in U.S. Pat. No. 7,041,807 related to YYX epitope; US20090175884, US20110305630, US20130330275 related to misfolded proteins in cancer; US20040175775 related to PrP in eye fluid; US20030114360 related to copolymers and methods of treating prion-related diseases; WO2009121176 related to insulin-induced gene peptide compositions; US20030022243, WO2003000853 related to protein aggregation assays; WO200078344 related to prion protein peptides and uses thereof. Each of these publications are incorporated by reference in their entireties.

In some embodiments, the viral genome of the AAV particles may comprise an Fc sequence which has been swapped with the Fc sequence of the reference antibody sequence, wherein the Fc swap may mediate direct cell killing.

### Antibody Generation

In some embodiments, viral genomes of the AAV particles may encode antibodies or antibody-based compositions produced using methods known in the art. Such methods may include, but are not limited to, immunization and display technologies (e.g., phage display, yeast display, and ribosomal display). Antibodies may be developed, for example, using any naturally occurring or synthetic antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

In some embodiments, the sequences of the polypeptides to be encoded in the viral genomes may be derived from antibodies produced using hybridoma technology. Host animals (e.g. mice, rabbits, goats, and llamas) may be immunized by an injection with an antigenic protein to elicit lymphocytes that specifically bind to the antigen. Lymphocytes may be collected and fused with immortalized cell lines to generate hybridomas which can be cultured in a suitable culture medium to promote growth. The antibodies produced by the cultured hybridomas may be subjected to analysis to determine binding specificity of the antibodies for the target antigen. Once antibodies with desirable characteristics are identified, corresponding hybridomas may be subcloned through limiting dilution procedures and grown by standard methods. The antibodies produced by these cells may be isolated and purified using standard immunoglobulin purification procedures.

In some embodiments, the sequences of the polypeptides to be encoded in the viral genomes may be produced using heavy and light chain variable region cDNA sequences selected from hybridomas or from other sources. Sequences encoding antibody variable domains expressed by hybridomas may be determined by extracting RNA molecules from antibody-producing hybridoma cells and producing cDNA by reverse transcriptase polymerase chain reaction (PCR). PCR may be used to amplify cDNA using primers specific for heavy and light chain sequences. PCR products may then be subcloned into plasmids for sequence analysis. Antibodies may be produced by insertion of resulting variable domain sequences into expression vectors.

In some embodiments, the sequences of the polypeptides to be encoded in the viral genomes may be generated using display technologies. Display technologies used to generate polypeptides may include any of the display techniques (e.g. display library screening techniques) disclosed in International Patent Application No. WO2014074532, the contents of which are herein incorporated by reference in their entirety. In some embodiments, synthetic antibodies may be designed, selected, or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the CDRs. In some cases, scFvs are expressed as fusion proteins, linked to viral coat proteins (e.g. the N-terminus of the viral pill coat protein). V_{L} chains may be expressed separately for assembly with V_{H} chains in the periplasm prior to complex incorporation into viral coats. Precipitated library members may be sequenced from the bound phage to obtain cDNA encoding desired scFvs. Antibody variable domains or CDRs from such sequences may be directly incorporated into antibody sequences for recombinant antibody production or mutated and utilized for further optimization through in vitro affinity maturation.

In some embodiments, the sequences of the polypeptides to be encoded in the viral genomes may be produced using yeast surface display technology, wherein antibody variable domain sequences may be expressed on the cell surface of Saccharomyces cerevisiae. Recombinant antibodies may be developed by displaying the antibody fragment of interest as a fusion to e.g. Aga2p protein on the surface of the yeast, where the protein interacts with proteins and small molecules in a solution. scFvs with affinity toward desired receptors may be isolated from the yeast surface using magnetic separation and flow cytometry. Several cycles of yeast surface display and isolation may be done to attain scFvs with desired properties through directed evolution.

In some embodiments, the sequence of the polypeptides to be encoded in the viral genomes (e.g., antibodies) may be designed by VERSITOPE™ Antibody Generation and other methods used by BIOATLA® and described in United States Patent Publication No. US20130281303, the contents of which are herein incorporated by reference in their entirety. In brief, recombinant monoclonal antibodies are derived from B-cells of a host immuno-challenged with one or more target antigens. These methods of antibody generation do not rely on immortalized cell lines, such as hybridoma, thereby avoiding some of the associated challenges i.e., genetic instability and low production capacity, producing high affinity and high diversity recombinant monoclonal antibodies. In some embodiments, the method is a natural diversity approach. In another embodiment, the method is a high diversity approach.

In some embodiments, the sequences of the polypeptides to be encoded in the viral genomes may be generated using the BIOATLA® natural diversity approach. In the natural diversity approach of generating recombinant monoclonal antibodies described in United States Patent Publication No. US20130281303, the original pairings of variable heavy (V_{H}) and variable light (V_{L}) domains are retained from the host, yielding recombinant monoclonal antibodies that are naturally paired. These may be advantageous due to a higher likelihood of functionality as compared to non-natural pairings of V_{H} and V_{L}. To produce the recombinant monoclonal antibodies, first a non-human host (i.e., rabbit, mouse, hamster, guinea pig, camel or goat) is immuno-challenged with an antigen of interest. In some embodiments, the host may be a previously challenged human patient. In other embodiments, the host may not have been immuno-challenged. B-cells are harvested from the host and screened by fluorescence activated cell sorting (FACS), or other method, to create a library of B-cells enriched in B-cells capable of binding the target antigen. The cDNA obtained from the mRNA of a single B-cell is then amplified to generate an immunoglobulin library of V_{H} and V_{L} domains. This library of immunoglobulins is then cloned into expression vectors capable of expressing the V_{H} and V_{L} domains, wherein the V_{H} and V_{L} domains remain naturally paired. The library of expression vectors is then used in an expression system to express the V_{H} and V_{L} domains in order to create an antibody library. Screening of the antibody library yields antibodies able to bind the target antigen, and these antibodies can be further characterized. Characterization may include one or more of the following: isoelectric point, thermal stability, sedimentation rate, folding rate, neutralization or antigen activity, antagonist or agonistic activity, expression level, specific and non-specific binding, inhibition of enzymatic activity, rigidity/flexibility, shape, charge, stability across pH, in solvents, under UV radiation, in mechanical stress conditions, or in sonic conditions, half-life, and glycosylation.

In some embodiments, the sequences of the polypeptides to be encoded in the viral genomes may be generated using the BIOATLA® high diversity approach. In the high diversity approach of generating recombinant monoclonal antibodies described in United States Patent Publication No. US20130281303, additional pairings of variable heavy (V_{H}) and variable light (V_{L}) domains are attained. To produce the recombinant monoclonal antibodies, B-cells harvested from the host are screened by fluorescence activated cell sorting (FACS), panning, or other method, to create a library of B-cells enriched in B-cells capable of binding the target antigen. The cDNA obtained from the mRNA of the pooled B-cells is then amplified to generate an immunoglobulin library of V_{H} and V_{L} domains. This library of immunoglobulins is then used in a biological display system (mammalian, yeast or bacterial cell surface display systems) to generate a population of cells displaying antibodies, fragments or derivatives comprising the V_{H} and V_{L} domains wherein, the antibodies, fragments or derivatives comprise V_{H} and V_{L} domain combinations that were not present in the B-cells in vivo. Screening of the cell population by FACS, with the target antigen, yields a subset of cells capable of binding the target antigen and the antibodies displayed on these cells can be further characterized. In an alternate embodiment of the high diversity approach, the immunoglobulin library comprises only V_{H} domains obtained from the B-cells of the immuno-challenged host, while the V_{L} domain(s) are obtained from another source.

In some embodiments, the sequences of the polypeptides to be encoded in the viral genomes may be evolved using BIOATLA® comprehensive approaches. The methods of generating recombinant monoclonal antibodies as described in United States Patent Publication No. US20130281303, further comprises evolving the recombinant antibody by comprehensive positional evolution (CPE™), CPE™ followed by comprehensive protein synthesis (CPS™), PCR shuffling, or other method.

In some embodiments, the sequence of the polypeptides to be encoded in the viral genomes (e.g., antibodies) may be derived from any of the BIOATLA® protein evolution methods described in International Publication WO2012009026, the contents of which are herein incorporated by reference in their entirety. In this method, mutations are systematically performed throughout the polypeptide or molecule of interest, a map is created providing useful informatics to guide the subsequent evolutionary steps. Not wishing to be bound by theory, these evolutionary methods typically start with a template polypeptide and a mutant is derived therefrom, which has desirable properties or characteristics. Non-limiting examples of evolutionary techniques include polymerase chain reaction (PCR), error prone PCR, oligonucleotide-directed mutagenesis, cassette mutagenesis, shuffling, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof.

In some embodiments, the BIOATLA® evolution method is Comprehensive Positional Evolution (CPE™). In CPE, naturally occurring amino acid variants are generated for each of the codons of the template polypeptide, wherein 63 different codon options exist for each amino acid variant. A set of polypeptides with single amino acid mutations are generated and the mutations are then confirmed by sequencing or other method known in the art and each amino acid change screened for improved function, neutral mutations, inhibitory mutations, expression, and compatibility with the host system. An EvoMap™ is created that describes in detail the effects of each amino acid mutation on the properties and characteristics of that polypeptide. The data from the EvoMap™ may be utilized to produce polypeptides with more than one amino acid mutation, wherein the resultant multi-site mutant polypeptides can be screened for desirable characteristics.

In some embodiments, the BIOATLA® evolution method is Synergy Evolution, wherein an EvoMap™ is used to identify amino acid positions to introduce 2-20 mutations simultaneously to produce a combinatorial effect. The resulting multi-site mutant polypeptides may be screened on one or more pre-determined characteristics to identify “upmutants” wherein the function of the mutant is improved as compared to the parent polypeptide. In some embodiments, Synergy Evolution is used to enhance binding affinity of an antibody.

In some embodiments, the BIOATLA® evolution method is Flex Evolution, wherein an EvoMap™ is used to identify fully mutable sites within a polypeptide that may then be targeted for alteration, such as introduction of glycosylation sites or chemical conjugation.

In some embodiments, the BIOATLA® evolution method is Comprehensive Positional Insertion Evolution (CPI™), wherein an amino acid is inserted after each amino acid of a template polypeptide to generate a set of lengthened polypeptides. CPI may be used to insert 1, 2, 3, 4, or 5 amino acids at each new position. The resultant lengthened polypeptides are sequenced and assayed for one or more pre-determined properties and evaluated in comparison to its template or parent molecule. In some embodiments, the binding affinity and immunogenicity of the resultant polypeptides are assayed. In some embodiments, the lengthened polypeptides are further mutated and mapped to identify polypeptides with desirable characteristics.

In some embodiments, the BIOATLA® evolution approach is Comprehensive Positional Deletion Evolution (CPD™), wherein each amino acid of the template polypeptide is individually and systematically deleted one at a time. The resultant shortened polypeptides are then sequenced and evaluated by assay for at least one pre-determined feature. In some embodiments, the shortened polypeptides are further mutated and mapped to identify polypeptides with desirable characteristics.

In some embodiments, the BIOATLA® evolution approach is Combinatorial Protein Synthesis (CPS™), wherein mutants identified in CPE, CPI, CPD, or other evolutionary techniques are combined for polypeptide synthesis. These combined mutant polypeptides are then screened for enhanced properties and characteristics. In some embodiments CPS is combined with any of the aforementioned evolutionary or polypeptide synthesis methods.

In some embodiments, the sequence of the polypeptides to be encoded in the viral genomes (e.g., antibodies) may be derived from the BIOATLA® Comprehensive Integrated Antibody Optimization (CIAO!™) described in U.S. Pat. No. 8,859,467, the contents of which are herein incorporated by reference in their entirety. The CIAO!™ method allows for simultaneous evolution of polypeptide performance and expression optimization, within a eukaryotic cell host (i.e., mammalian or yeast cell host). First, an antibody library is generated in a mammalian cell production host by antibody cell surface display, wherein the generated antibody library targets a particular antigen of interest. The antibody library is then screened by any method known in the art, for one or more properties or characteristics. One or more antibodies of the library, with desirable properties or characteristics are chosen for further polypeptide evolution by any of the methods known in the art, to produce a library of mutant antibodies by antibody cell surface display in a mammalian cell production host. The generated mutant antibodies are screened for one or more predetermined properties or characteristics, whereby an upmutant is selected, wherein the upmutant has enhanced or improved characteristics as compared to the parent template polypeptide.

In some embodiments, the sequences of the polypeptides to be encoded in the viral genomes may be humanized by the methods of BIOATLA® as described in United States Patent Publication US20130303399, the contents of which are herein incorporated by reference in their entirety. In this method, for generating enhanced full-length humanized antibodies in mammalian cells, no back-mutations are required to retain affinity to the antigen and no CDR grafting or phage-display is necessary. The generated humanized antibody has reduced immunogenicity and equal or greater affinity for the target antigen as compared to the parent antibody. The variable regions or CDRs of the generated humanized antibody are derived from the parent or template, whereas the framework and constant regions are derived from one or more human antibodies. To start, the parent, or template antibody is selected, cloned and each CDR sequence identified and synthesized into a CDR fragment library. Double stranded DNA fragment libraries for V_{H} and V_{L} are synthesized from the CDR fragment encoding libraries, wherein at least one CDR fragment library is derived from the template antibody and framework (FW fragment encoding libraries, wherein the FW fragment library is derived from a pool of human frameworks obtained from natively expressed and functional human antibodies. Stepwise liquid phase ligation of FW and CDR encoding fragments is then used to generate both V_{H} and V_{L} fragment libraries. The V_{H} and V_{L} fragment libraries are then cloned into expression vectors to create a humanization library, which is further transfected into cells for expression of full length humanized antibodies and used to create a humanized antibody library. The humanized antibody library is then screened to determine expression level of the humanized antibodies, affinity or binding ability for the antigen, and additional improved or enhanced characteristics, as compared to the template or parent antibody. Non-limiting examples of characteristics that may be screened include equilibrium dissociation constant (K_{D}), stability, melting temperature (Tₘ), pl, solubility, expression level, reduced immunogenicity, and improved effector function.

In some embodiments, the sequences of the polypeptides to be encoded in the viral genomes may be generated by the BIOATLA® method for preparing conditionally active antibodies as described in International Publications WO2016033331 and WO2016036916, the contents of which are herein incorporated by reference in their entirety. As used herein, the term “conditionally active” refers to a molecule that is active at an aberrant condition. Further, the conditionally active molecule may be virtually inactive at normal physiological conditions. Aberrant conditions may result from changes in pH, temperature, osmotic pressure, osmolality, oxidative stress, electrolyte concentration, and/or chemical or proteolytic resistance, as non-limiting examples.

The method of preparing a conditionally active antibody is described in International Publications WO2016033331 and WO2016036916 and summarized herein. Briefly, a wild-type polypeptide is selected and the DNA is evolved to create mutant DNAs. Non-limiting examples of evolutionary techniques that may be used to evolve the DNA include polymerase chain reaction (PCR), error prone PCR, shuffling, oligonucleotide-directed mutagenesis, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof. Once mutant DNAs are created, they are expressed in a eukaryotic cell production host (i.e., fungal, insect, mammalian, adenoviral, plant), wherein a mutant polypeptide is produced. The mutant polypeptide and the corresponding wild-type polypeptide are then subjected to assays under both normal physiological conditions and aberrant conditions in order to identify mutants that exhibit a decrease in activity in the assay at normal physiological conditions as compared to the wild-type polypeptide and/or an increase in activity in the assay under aberrant conditions, as compared to the corresponding wild-type polypeptide. The desired conditionally active mutant may then be produced in the aforementioned eukaryotic cell production host.

In some embodiments, the conditionally active antibody is a “mirac protein” as described by BIOATLA® in U.S. Pat. No. 8,709,755, the contents of which are herein incorporated by reference in their entirety. As used herein “mirac protein” refers to a conditionally active antibody that is virtually inactive at body temperature but active at lower temperatures.

In some embodiments, the sequence of the polypeptides to be encoded in the viral genomes (e.g., antibodies) may be derived based on any of the BIOATLA™ methods including, but not limited to, VERSITOPE™ Antibody Generation, natural diversity approaches, and high diversity approaches for generating monoclonal antibodies, methods for generation of conditionally active polypeptides, humanized antibodies, mirac proteins, multi-specific antibodies or cross-species active mutant polypeptides, Comprehensive Integrated Antibody Optimization (CIAO!™), Comprehensive Positional Evolution (CPE™), Synergy Evolution, Flex Evolution, Comprehensive Positional Insertion Evolution (CPI™), Comprehensive Positional Deletion Evolution (CPD™), Combinatorial Protein Synthesis (CPS™), or any combination thereof. These methods are described in United States Patent Nos. U.S. Pat. Nos. 8,859,467 and 8,709,755 and United States Publication Nos. US20130281303, US20130303399, US20150065690, US20150252119, US20150086562 and US20100138945, and International Publication Nos. WO2015105888, WO2012009026, WO2011109726, WO2016036916, and WO2016033331, the contents of each of which are herein incorporated by reference in their entirety.

In some embodiments, antibodies of the present disclosure are generated by any of the aforementioned means to target one or more of the following epitopes of the tau protein; phosphorylated tau peptides, pS396, pS396-pS404, pS404, pS396-pS404-pS422, pS422, pS199, pS199-pS202, pS202, pT181, pT231, cis-pT231, any of the following acetylated sites acK174, acK274, acK280, acK281 and/or any combination thereof.

### Antibody Fragments and Variants

In some embodiments, antibody fragments encoded by payloads comprise antigen binding regions from intact antibodies. Examples of antibody fragments may include, but are not limited to Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Compounds and/or compositions of the present disclosure may comprise one or more of these fragments. For the purposes herein, an “antibody” may comprise a heavy and light variable domain as well as an Fc region.

In some embodiments, the Fc region may be a modified Fc region, as described in US Patent Publication US20150065690, wherein the Fe region may have a single amino acid substitution as compared to the corresponding sequence for the wild-type Fc region, wherein the single amino acid substitution yields an Fc region with preferred properties to those of the wild-type Fc region. Non-limiting examples of Fc properties that may be altered by the single amino acid substitution include bind properties or response to pH conditions

As used herein, the term “native antibody” refers to a usually heterotetrameric glycoprotein of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602.9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia Pa. 2012. Ch. 3, p 47-54, the contents of which are herein incorporated by reference in their entirety). Determining residues making up CDRs may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and Al-Lazikani, B. et al., 1997, J Mol Biol. 273(4):927-48, the contents of each of which are herein incorporated by reference in their entirety], Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

V_{H} and V_{L} domains have three CDRs each. V_{L} CDRs are referred to herein as CDR-L1, CDR-L2 and CDR-L3, in order of occurrence when moving from N- to C-terminus along the variable domain polypeptide. V_{H} CDRs are referred to herein as CDR-H1, CDR-H2, and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. Peer J. 2:e456; the contents of which are herein incorporated by reference in their entirety). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia Pa. 2012. Ch. 3, p 46.47, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of V_{H} and V_{L} antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

As used herein, the term “diabody” refers to a small antibody fragment with two antigen-binding sites. Diabodies comprise a heavy chain variable domain V_{H} connected to alight chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404097; WO 9311161; and Hollinger et al. (Hollinger, P. et al., “Diabodies”: Small bivalent and bispecific antibody fragments. PNAS. 1993. 90:6444-8) the contents of each of which are incorporated herein by reference in their entirety.

The term “Intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present disclosure may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

In some embodiments, viral genomes of the present disclosure may encode antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, Centyrins, DARPINS™, fynomers, Kunitz domains, and domain peptides. In other embodiments, antibody mimetics may include one or more non-peptide regions.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition, or structure as compared to a native antibody. Antibody variants may include, but are not limited to, antibodies with altered isotypes (e.g., IgA, IgD, IgE, IgG₁, IgG₂, IgG₃, IgG₄, or IgM), humanized variants, optimized variants, multispecific antibody variants (e.g., bispecific variants), and antibody fragments.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

### Multispecific Antibodies

In some embodiments, payloads may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

In some embodiments, multi-specific antibodies may be prepared by the methods used by BIOATLA® and described in International Patent publication WO201109726, the contents of which are herein incorporated by reference in their entirety. First a library of homologous, naturally occurring antibodies is generated by any method known in the art (i.e., mammalian cell surface display), then screened by FACSAria or another screening method, for multi-specific antibodies that specifically bind to two or more target antigens. In some embodiments, the identified multi-specific antibodies are further evolved by any method known in the art, to produce a set of modified multi-specific antibodies. These modified multi-specific antibodies are screened for binding to the target antigens. In some embodiments, the multi-specific antibody may be further optimized by screening the evolved modified multi-specific antibodies for optimized or desired characteristics.

In some embodiments, multi-specific antibodies may be prepared by the methods used by BIOATLA® and described in Unites States Publication No. US20150252119, the contents of which are herein incorporated by reference in their entirety. In one approach, the variable domains of two parent antibodies, wherein the parent antibodies are monoclonal antibodies are evolved using any method known in the art in a manner that allows a single light chain to functionally complement heavy chains of two different parent antibodies. Another approach requires evolving the heavy chain of a single parent antibody to recognize a second target antigen. A third approach involves evolving the light chain of a parent antibody so as to recognize a second target antigen. Methods for polypeptide evolution are described in International Publication WO2012009026, the contents of which are herein incorporated by reference in their entirety, and include as non-limiting examples, Comprehensive Positional Evolution (CPE), Combinatorial Protein Synthesis (CPS), Comprehensive Positional Insertion (CPI), Comprehensive Positional Deletion (CPD), or any combination thereof. The Fc region of the multi-specific antibodies described in United States Publication No. US20150252119 may be created using a knob-in-hole approach, or any other method that allows the Fc domain to form heterodimers. The resultant multi-specific antibodies may be further evolved for improved characteristics or properties such as binding affinity for the target antigen.

### Bispecific Antibodies

In some embodiments, payloads may encode bispecific antibodies. Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

In some cases, payloads encode bispecific antibodies comprising antigen-binding regions from two different antibodies. For example, such bispecific antibodies may comprise binding regions from two different antibodies selected from Tables 3-16.

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications and are described as “Bispecific T-cell Engagers (BITE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs. 2010. January-February; 2(1):77-83).

In some embodiments, payloads may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (V_{HH}s) of heavy chain antibodies found in camels and llamas, which lack light chains (Nelson, A. L., MAbs. 2010. January-February; 2(1):77-83). Nanobodies are single heavy chain antibodies. In some embodiments, nanobodies may have a high solubility and a molecular weight that is lower than an antibody. In some embodiments, nanobodies may exhibit high stability in the presence of strong denaturing agents and/or extreme pH environments—conditions which may cause the degradation of full length antibodies. Nanobodies possess high affinity and specificity. Compared to antibodies, nanobodies may have a longer CDR3 (complementarity-determining region 3) which may form a binding surface that is stable, and convex relative to the concave or planar antigen-binding surface of an antibody. Nanobodies may possess weak immunogenicity and strong penetrability. The immunogenicity may be related to the size and chemical structure of the nanobodies. The small size of the nanobodies may also result in strong tissue penetrating ability.

In some embodiments, the nanobodies may be bispecific nanobodies.

Disclosed and claimed in PCT Publication WO2014144573 to Memorial Sloan-Kettering Cancer Center are multimerization technologies for making dimeric multispecific binding agents (e.g., fusion proteins comprising antibody components) with improved properties over multispecific binding agents without the capability of dimerization.

In some cases, payloads may encode tetravalent bispecific antibodies (TetBiAbs as disclosed and claimed in PCT Publication WO2014144357). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, payloads may encode biosynthetic antibodies as described in U.S. Pat. No. 5,091,513, the contents of which are herein incorporated by reference in their entirety. Such antibody may include one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L} are attached by a polypeptide linker, or 3) individuals V_{H} or V_{L} domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, payloads may encode antibodies with antibody acceptor frameworks taught in U.S. Pat. No. 8,399,625. Such antibody acceptor frameworks may be particularly well suited accepting CDRs from an antibody of interest. In some cases, CDRs from anti-tau antibodies known in the art or developed according to the methods presented herein may be used.

### Miniaturized Antibody

In some embodiments, the antibody encoded by the payloads may be a “miniaturized” antibody. Among the best examples of mAb miniaturization are the small modular immune pharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphold neoplasia, a project that has reached Phase 2. Wyeth has licensed the anti-CD20 SMIP SBI-087 for the treatment of autoimmune diseases, including RA, SLE, and possibly multiple sclerosis, although these projects remain in the earliest stages of clinical testing. (Nelson, A. L., MAbs. 2010. January-February; 2(1):77-83).

### Diabodies

In some embodiments, payloads may encode diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs., 2010. January-February; 2(1):77-83).

### Unibody

In some embodiments, payloads may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies (see, e.g., Nelson, A. L., MAbs., 2010. January-February; 2(1):77-83).

### Intrabodies

In some embodiments, payloads may encode intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein. DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes using methods known in the art, such as those disclosed and reviewed in: (Marasco et al., 1993 Proc. Natl. Acad. Sci. USA, 90: 7889-7893; Chen et al, 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 119; Mhashilkar, et al., 1995, EMBO J, 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol, 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805.810). See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated V_{H} and V_{L} domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and can modulate the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its Nor C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif (SEQ ID NO: 13163). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly synthesized intrabody within the cell is affected by reducing conditions of the intracellular environment.

Intrabodies may be promising therapeutic agents for the treatment of misfolding diseases, including Tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

### Maxibodies

In some embodiments, the payloads encode a maxibody (bivalent scFv fused to the amino terminus of the Fc (CH2-CH3 domains) of IgG.

### Chimeric Antigen Receptors

In some embodiments, the polypeptides encoded by the viral genomes (e.g., antibodies) may be used to generate chimeric antigen receptors (CARs) as described by BIOATLA® in International Publications WO2016033331 and WO2016036916, the contents of which are herein incorporated by reference in their entirety. As used herein, a “chimeric antigen receptor (CAR)” refers to an artificial chimeric protein comprising at least one antigen specific targeting region (ASTIR), wherein the antigen specific targeting region comprises a full-length antibody or a fragment thereof that specifically binds to a target antigen. The ASTR may comprise any of the following: a full length heavy or light chain, an Fab fragment, a single chain Fv fragment, a divalent single chain antibody, or a diabody. As a non-limiting example, the ASTR of a CAR may be any of the antibodies listed in Tables 3-16, antibody-based compositions or fragments thereof. Any molecule that is capable of binding a target antigen with high affinity can be used in the ASTR of a CAR. In some embodiments, the CAR may have more than one ASTR. These ASTRs may target two or more antigens or two or more epitopes of the same antigen. In some embodiments, the CAR is conditionally active. In some embodiments, the CAR is used to produce a genetically engineered cytotoxic cell carrying the CAR and capable of targeting the antigen bound by the ASTR.

Chimeric antigen receptors (CARs) are particularly useful in the treatment of cancers, though also therapeutically effective in treatment of a wide variety of other diseases and disorders. Non-limiting examples of disease categories that may be treated with CARs or CAR-based therapeutics include autoimmune disorders, B-cell mediated diseases, inflammatory diseases, neuronal disorders, cardiovascular disease and circulatory disorders, or infectious diseases. Not wishing to be bound by theory, CARs traditionally work by targeting antigens presented on the surface of or on the inside of cells to be destroyed e.g., cancer tumor cells, by the cytotoxic cell of the CAR.

In some embodiments, payloads of the present disclosure may be a chimeric antigen receptor (CAR), which when transduced into immune cells (e.g., T cells and NK cells), can re-direct the immune cells against the target (e.g., a tumor cell) which expresses a molecule recognized by the extracellular target moiety of the CAR as described in U.S. provisional patent application 62/844,433, the contents of which are herein incorporated by reference in their entirety.

### Senescent Cell Surface Protein Antibodies

In some embodiments, the AAV particles may comprise nucleic acids which have been engineered to express of antibodies that selectively bind to surface marker proteins of senescent cells. For example, the antibodies may selectively bind to proteins that are in misfolded conformation. The binding antibodies may reduce the number of senescent cells and be used to treat age-related conditions, such as, but not limited to, Alzheimer's disease, cardiovascular disease, emphysema, sarcopenia, and tumorigenesis as well as conditions more cosmetic in nature such as signs of skin aging including wrinkling, sagging, discoloration, age-related tissue dysfunction, tumor formation, and other age-related conditions.

In some embodiments, the expressed antibodies binding to epitopes of senescent cell surface proteins may be, but are not limited to, such as prion epitopes presented by SEQ ID NO: 1-14 of International Publication No. WO2014186878; CD44 epitopes presented by SEQ ID NO: 47.51 of International Publication No. WO2014186878; TNFR epitopes presented by SEQ ID NO: 52.56 of International Publication No. WO2014186878; NOTCH1 epitope presented by SEQ ID NO: 57-61 of International Publication No. WO2014186878; FasR epitopes presented by SEQ ID NO: 62-66 of International Publication No. WO2014186878; epidermal growth factor epitopes presented by SEQ ID NO: 67-81 of International Publication No. WO2014186878; CD38 epitopes presented by SEQ ID NO: 82-86 of International Publication No. WO2014186878, the contents of each of which are herein incorporated by reference in their entirety.

In some embodiments, the expressed antibodies may comprise peptides binding to senescent cell surface prion proteins, such as, but not limited to, those presented by SEQ ID NO: 15-36 of International Publication No. WO2014186878, the contents of which are herein incorporated by reference in their entirety.

In some embodiments, the expressed antibody may be AMF-3a-118 or AMF 3d-19 (SEQ ID NO: 89-92 and 103-106 of International publication WO2014186878, respectively, the contents of which are herein incorporated by reference in their entirety) targeting senescent cell surface protein FasR. In some embodiments, the expressed antibody may be Ab c-120 (SEQ ID NO: 37-40 of International publication WO2014186878, the contents of which are herein incorporated by reference in their entirety) targeting senescent cell surface protein PrP.

### Antibody Drug Conjugates

In certain embodiments, a therapeutic molecule comprises an antibody conjugated to an oligonucleotide with a linker. The antibody may engage a highly expressed receptor expressed on the surface of a cell type of interest, for example, a muscle cell. The muscle cell may be skeletal, cardiac, or smooth muscle. In some embodiments, the receptor may be expressed only on cells with a disease. In some embodiments, the receptor may be expressed only on the cell type of interest. The disease may be myotonic dystrophy Type 1 (DM1). By the antibody engaging the receptor, the oligonucleotide is brought to the primary site of disease to facilitate delivery of the oligonucleotide into the cell type of interest. The antibody-oligonucleotide conjugate is wholly engulfed by the cell. Once inside the cell, the oligonucleotide binds with the RNA that is driving disease progression, thereby degrading the disease-causing RNA. In some embodiments, the therapeutic molecule increases the delivery specificity of the oligonucleotide compared to present delivery methods of an oligonucleotide. In certain embodiments, administering the therapeutic molecule to a subject results in decreased systemic effects compared to present delivery methods of an oligonucleotide.

### Payload Antibodies of the Disclosure

The payload region of the AAV particle described in the present disclosure may comprise one or more nucleic acid sequences encoding antibodies, variants or fragments thereof. In some embodiments, the payload region of the AAV particle comprises one or more nucleic acid sequences encoding infectious disease antibodies, variants or fragments thereof. As a non-limiting example, the payload region of the AAV particle comprises one or more nucleic acid sequence encoding infectious disease antibodies targeting John Cunningham Virus, Influenza virus, Hepatitis, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Pseudomonas Aeruginosa, Streptococcus bacteria, Staphylococcus bacteria, Clostridium Tetani, Bordetella, Mycobacterium, Francisella Tularensis, Toxoplasma gondii, Candida yeast, Human Immunodeficiency Virus (HIV),Plasmodium falciparum, Ebola virus, Marburg virus, West Nile virus, Yellow Fever virus, Japanese encephalitis virus, St. Louis encephalitis virus, Chikungunya virus, Dengue virus, Trypanosoma cruzi, Rabies virus, Rotavirus, Norwalk virus/Norovirus, Campylobacter jejuni, Clostridium difficile, Entamoeba histolytica, Helicobacter pylori, Enterotoxin B, Ricin, Becillus anthracis, Shiga and Shiga-like toxins, and Botulinum toxins. As another non-limiting example, the payload region of the AAV particle may be any of the infectious disease antibodies listed in Table 3.

In certain embodiments, the payload region of the AAV particle comprises one or more nucleic acid sequences encoding non-infectious disease antibodies, variants or fragments thereof. As a non-limiting example, the payload region of the AAV particle comprises one or more nucleic acid sequence encoding non-infectious disease antibodies targeting cancer, immune diseases, inflammatory disorders, blood and blood vessel diseases, respiratory diseases, muscle diseases, bone diseases, endocrine and metabolic diseases, nervous system diseases, e.g., Alzheimer's disease, Parkinson's disease, Dementia with Lewy bodies, Huntington's disease, Amyotrophic lateral sclerosis, multiple sclerosis, multiple systems atrophy, spinal muscular atrophy, neuropathies, psychiatric disorders, migraine, pain, and ocular diseases. As another non-limiting example, the payload region of the AAV particle may be any of the non-infectious disease antibodies listed in Tables 4-15.

### Payload Antibodies: Infectious Disease

In some embodiments, the payload region of the AAV particle comprises one or more nucleic acid sequences encoding infectious disease-associated antibodies, variants or fragments thereof.

In some embodiments, the payload region of the AAV particle comprises one or more nucleic acid sequences encoding one or more of the payload antibody polypeptides listed in Table 3, or variants or fragments thereof. As used herein, “antibody polynucleotide” refers to a nucleic acid sequence encoding an antibody polypeptide.

In some embodiments, the payload region of the AAV particle comprises one or more nucleic acid sequences listed in Table 3, or variants or fragments thereof.

In some embodiments, the payload region of the AAV particle comprises a nucleic acid sequence encoding a payload antibody with at least 50% identity to one or more payload antibody polypeptides listed in Table 3. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the full sequence of the encoded antibody polypeptide may have 50%, 51%, 52%, 53%, 54%, 55%, 56%, 57%, 58%, 59%,60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%,87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the heavy chain of the encoded antibody polypeptide may have 50%, 51%,52%, 53%,54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 3, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 3, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%,76%,77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 3, or variants or fragments thereof.

In some embodiments, the payload antibody has 90% identity to one or more of the antibody polypeptides listed in Table 3, or variants or fragments thereof.

In some embodiments, the payload antibody has 91% identity to one or more of the antibody polypeptides listed in Table 3, or variants or fragments thereof.

In some embodiments, the payload antibody has 92% identity to one or more of the antibody polypeptides listed in Table 3, or variants or fragments thereof.

In some embodiments, the payload antibody has 93% identity to one or more of the antibody polypeptides listed in Table 3, or variants or fragments thereof.

In some embodiments, the payload antibody has 94% identity to one or more of the antibody polypeptides listed in Table 3, or variants or fragments thereof.

In some embodiments, the payload antibody has 95% identity to one or more of the antibody polypeptides listed in Table 3, or variants or fragments thereof.

In some embodiments, the payload antibody has 96% identity to one or more of the antibody polypeptides listed in Table 3, or variants or fragments thereof.

In some embodiments, the payload antibody has 97% identity to one or more of the antibody polypeptides listed in Table 3, or variants or fragments thereof.

In some embodiments, the payload antibody has 98% identity to one or more of the antibody polypeptides listed in Table 3, or variants or fragments thereof.

In some embodiments, the payload antibody has 99% identity to one or more of the antibody polypeptides listed in Table 3, or variants or fragments thereof.

In some embodiments, the payload antibody has 100% identity to one or more of the antibody polypeptides listed in Table 3, or variants or fragments thereof.

In some embodiments, the payload region of the AAV particle comprises a nucleic acid sequence with at least 50% identity to one or more nucleic acid sequences listed in Table 3, or variants or fragments thereof. The payload nucleic acid sequence may have 50%, 51%,52%,53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 3, or variants or fragments thereof.

In some embodiments, the payload nucleic acid sequence has 90% identity to one or more of the nucleic acid sequences listed in Table 3, or variants or fragments thereof.

In some embodiments, the payload nucleic acid sequence has 91% identity to one or more of the nucleic acid sequences listed in Table 3, or variants or fragments thereof.

In some embodiments, the payload nucleic acid sequence has 92% identity to one or more of the nucleic acid sequences listed in Table 3, or variants or fragments thereof.

In some embodiments, the payload nucleic acid sequence has 93% identity to one or more of the nucleic acid sequences listed in Table 3, or variants or fragments thereof.

In some embodiments, the payload nucleic acid sequence has 94% identity to one or more of the nucleic acid sequences listed in Table 3, or variants or fragments thereof.

In some embodiments, the payload nucleic acid sequence has 95% identity to one or more of the nucleic acid sequences listed in Table 3, or variants or fragments thereof.

In some embodiments, the payload nucleic acid sequence has 96% identity to one or more of the nucleic acid sequences listed in Table 3, or variants or fragments thereof.

In some embodiments, the payload nucleic acid sequence has 97% identity to one or more of the nucleic acid sequences listed in Table 3, or variants or fragments thereof.

In some embodiments, the payload nucleic acid sequence has 98% identity to one or more of the nucleic acid sequences listed in Table 3, or variants or fragments thereof.

In some embodiments, the payload nucleic acid sequence has 99% identity to one or more of the nucleic acid sequences listed in Table 3, or variants or fragments thereof.

In some embodiments, the payload nucleic acid sequence has 100% identity to one or more of the nucleic acid sequences listed in Table 3, or variants or fragments thereof.

In some embodiments, the payload antibody may be variants of any of the antibody polypeptides listed in Table 3, that exclude one or more amino acids designated as “X” or “x” in the described polypeptide sequence, wherein X may represent any amino acid. In some embodiments, the payload nucleic acid sequence may be variants of any of the nucleic acid sequences listed in Table 3, that exclude one or more nucleic acids designated as “n” or “N” in the described nucleic acid sequence, wherein n may represent any nucleic acid.

TABLE 3
Infectious disease antibodies

| | | Type | | SEQ |
|---|---|---|---|---|
| Ab ID | Component | (PRT/DNA) | Reference | ID NO. |
| IFD1 | CDR | DNA | U.S. Pat. No. 8,562,996; SEQ ID NO: 60 | 1740 |
| IFD2 | CDR | DNA | U.S. Pat. No. 8,562,996; SEQ ID NO: 97 | 1741 |
| IFD3 | CDR | DNA | U.S. Pat. No. 8,562,996; SEQ ID NO: 71 | 1742 |
| IFD4 | CDR | DNA | U.S. Pat. No. 8,562,996; SEQ ID NO: 67 | 1743 |
| IFD5 | CDR | DNA | U.S. Pat. No. 8,562,996; SEQ ID NO: 64 | 1744 |
| IFD6 | CDR | DNA | U.S. Pat. No. 8,562,996; SEQ ID NO: 95 | 1745 |
| IFD7 | CDR | DNA | U.S. Pat. No. 8,562,996; SEQ ID NO: 62 | 1746 |
| IFD8 | CDR | DNA | U.S. Pat. No. 8,562,996; SEQ ID NO: 69 | 1747 |
| IFD9 | CDR | DNA | US20190031747; SEQ ID NO: 101 | 1748 |
| IFD10 | CDR | DNA | US20190031747; SEQ ID NO: 245 | 1749 |
| IFD11 | CDR | DNA | US20190031747; SEQ ID NO: 21 | 1750 |
| IFD12 | CDR | DNA | US20190031747; SEQ ID NO: 53 | 1751 |
| IFD13 | CDR | DNA | US20190031747; SEQ ID NO: 293 | 1752 |
| IFD14 | CDR | DNA | US20190031747; SEQ ID NO: 309 | 1753 |
| IFD15 | CDR | DNA | US20190031747; SEQ ID NO: 229 | 1754 |
| IFD16 | CDR | DNA | US20190031747; SEQ ID NO: 69 | 1755 |
| IFD17 | CDR | DNA | US20190031747; SEQ ID NO: 85 | 1756 |
| IFD18 | CDR | DNA | US20190031747; SEQ ID NO: 181 | 1757 |
| IFD19 | CDR | DNA | US20190031747; SEQ ID NO: 213 | 1758 |
| IFD20 | CDR | DNA | US20190031747; SEQ ID NO: 319 | 1759 |
| IFD21 | CDR | DNA | US20190031747; SEQ ID NO: 255 | 1760 |
| IFD22 | CDR | DNA | US20190031747; SEQ ID NO: 159 | 1761 |
| IFD23 | CDR | DNA | US20190031747; SEQ ID NO: 341 | 1762 |
| IFD24 | CDR | DNA | US20190031747; SEQ ID NO: 197 | 1763 |
| IFD25 | CDR | DNA | US20190031747; SEQ ID NO: 117 | 1764 |
| IFD26 | CDR | DNA | US20190031747; SEQ ID NO: 165 | 1765 |
| IFD27 | CDR | DNA | US20190031747; SEQ ID NO: 149 | 1766 |
| IFD28 | CDR | DNA | US20190031747; SEQ ID NO: 107 | 1767 |
| IFD29 | CDR | DNA | US20190031747; SEQ ID NO: 315 | 1768 |
| IFD30 | CDR | DNA | US20190031747; SEQ ID NO: 79 | 1769 |
| IFD31 | CDR | DNA | US20190031747; SEQ ID NO: 15 | 1770 |
| IFD32 | CDR | DNA | US20190031747; SEQ ID NO: 111 | 1771 |
| IFD33 | CDR | DNA | US20190031747; SEQ ID NO: 351 | 1772 |
| IFD34 | CDR | DNA | US20190031747; SEQ ID NO: 239 | 1773 |
| IFD35 | CDR | DNA | US20190031747; SEQ ID NO: 191 | 1774 |
| IFD36 | CDR | DNA | US20190031747; SEQ ID NO: 251 | 1775 |
| IFD37 | CDR | DNA | US20190031747; SEQ ID NO: 219 | 1776 |
| IFD38 | CDR | DNA | US20190031747; SEQ ID NO: 347 | 1777 |
| IFD39 | CDR | DNA | US20190031747; SEQ ID NO: 139 | 1778 |
| IFD40 | CDR | DNA | US20190031747; SEQ ID NO: 11 | 1779 |
| IFD41 | CDR | DNA | US20190031747; SEQ ID NO: 155 | 1780 |
| IFD42 | CDR | DNA | US20190031747; SEQ ID NO: 203 | 1781 |
| IFD43 | CDR | DNA | US20190031747; SEQ ID NO: 91 | 1782 |
| IFD44 | CDR | DNA | US20190031747; SEQ ID NO: 123 | 1783 |
| IFD45 | CDR | DNA | US20190031747; SEQ ID NO: 223 | 1784 |
| IFD46 | CDR | DNA | US20190031747; SEQ ID NO: 207 | 1785 |
| IFD47 | CDR | DNA | US20190031747; SEQ ID NO: 95 | 1786 |
| IFD48 | CDR | DNA | US20190031747; SEQ ID NO: 127 | 1787 |
| IFD49 | CDR | DNA | US20190031747; SEQ ID NO: 143 | 1788 |
| IFD50 | CDR | DNA | US20190031747; SEQ ID NO: 299 | 1789 |
| IFD51 | CDR | DNA | US20190031747; SEQ ID NO: 187 | 1790 |
| IFD52 | CDR | DNA | US20190031747; SEQ ID NO: 59 | 1791 |
| IFD53 | CDR | DNA | US20190031747; SEQ ID NO: 27 | 1792 |
| IFD54 | CDR | DNA | US20190031747; SEQ ID NO: 75 | 1793 |
| IFD55 | CDR | DNA | US20190031747; SEQ ID NO: 235 | 1794 |
| IFD56 | CDR | DNA | US20190031747; SEQ ID NO: 303 | 1795 |
| IFD57 | CDR | DNA | US20190031747; SEQ ID NO: 31 | 1796 |
| IFD58 | CDR | DNA | US20190031747; SEQ ID NO: 37 | 1797 |
| IFD59 | CDR | DNA | US20190031747; SEQ ID NO: 63 | 1798 |
| IFD60 | CDR | DNA | US20190031747; SEQ ID NO: 119 | 1799 |
| IFD61 | CDR | DNA | US20190031747; SEQ ID NO: 231 | 1800 |
| IFD62 | CDR | DNA | US20190031747; SEQ ID NO: 167 | 1801 |
| IFD63 | CDR | DNA | US20190031747; SEQ ID NO: 71 | 1802 |
| IFD64 | CDR | DNA | US20190031747; SEQ ID NO: 247 | 1803 |
| IFD65 | CDR | DNA | US20190031747; SEQ ID NO: 39 | 1804 |
| IFD66 | CDR | DNA | US20190031747; SEQ ID NO: 23 | 1805 |
| IFD67 | CDR | DNA | US20190031747; SEQ ID NO: 295 | 1806 |
| IFD68 | CDR | DNA | US20190031747; SEQ ID NO: 327 | 1807 |
| IFD69 | CDR | DNA | US20190031747; SEQ ID NO: 55 | 1808 |
| IFD70 | CDR | DNA | US20190031747; SEQ ID NO: 103 | 1809 |
| IFD71 | CDR | DNA | US20190031747; SEQ ID NO: 87 | 1810 |
| IFD72 | CDR | DNA | US20190031747; SEQ ID NO: 183 | 1811 |
| IFD73 | CDR | DNA | US20190031747; SEQ ID NO: 215 | 1812 |
| IFD74 | CDR | DNA | US20190031747; SEQ ID NO: 311 | 1813 |
| IFD75 | CDR | DNA | US20190031747; SEQ ID NO: 199 | 1814 |
| IFD76 | CDR | DNA | US20190031747; SEQ ID NO: 343 | 1815 |
| IFD77 | CDR | DNA | US20190031747; SEQ ID NO: 7 | 1816 |
| IFD78 | CDR | DNA | US20190031747; SEQ ID NO: 3 | 1817 |
| IFD79 | CDR | DNA | US20190031747; SEQ ID NO: 83 | 1818 |
| IFD80 | CDR | DNA | US20190031747; SEQ ID NO: 51 | 1819 |
| IFD81 | CDR | DNA | US20190031747; SEQ ID NO: 67 | 1820 |
| IFD82 | CDR | DNA | US20190031747; SEQ ID NO: 339 | 1821 |
| IFD83 | CDR | DNA | US20190031747; SEQ ID NO: 19 | 1822 |
| IFD84 | CDR | DNA | US20190031747; SEQ ID NO: 243 | 1823 |
| IFD85 | CDR | DNA | US20190031747; SEQ ID NO: 35 | 1824 |
| IFD86 | CDR | DNA | US20190031747; SEQ ID NO: 195 | 1825 |
| IFD87 | CDR | DNA | US20190031747; SEQ ID NO: 147 | 1826 |
| IFD88 | CDR | DNA | US20190031747; SEQ ID NO: 99 | 1827 |
| IFD89 | CDR | DNA | US20190031747; SEQ ID NO: 115 | 1828 |
| IFD90 | CDR | DNA | US20190031747; SEQ ID NO: 163 | 1829 |
| IFD91 | CDR | DNA | US20190031747; SEQ ID NO: 291 | 1830 |
| IFD92 | CDR | DNA | US20190031747; SEQ ID NO: 323 | 1831 |
| IFD93 | CDR | DNA | US20190031747; SEQ ID NO: 179 | 1832 |
| IFD94 | CDR | DNA | US20190031747; SEQ ID NO: 211 | 1833 |
| IFD95 | CDR | DNA | US20190031747; SEQ ID NO: 307 | 1834 |
| IFD96 | CDR | DNA | US20190031747; SEQ ID NO: 263 | 1835 |
| IFD97 | CDR | DNA | US20190031747; SEQ ID NO: 135 | 1836 |
| IFD98 | CDR | DNA | US20190031747; SEQ ID NO: 279 | 1837 |
| IFD99 | CDR | DNA | US20190031747; SEQ ID NO: 151 | 1838 |
| IFD100 | CDR | DNA | US20190031747; SEQ ID NO: 133 | 1839 |
| IFD101 | CDR | DNA | US20190031747; SEQ ID NO: 5 | 1840 |
| IFD102 | CDR | PRT | US20190015509; SEQ ID NO: 9 | 1841 |
| IFD103 | CDR | PRT | US20190015509; SEQ ID NO: 10 | 1842 |
| IFD104 | CDR | PRT | US20190015509; SEQ ID NO: 8 | 1843 |
| IFD105 | CDR | PRT | US20190015509; SEQ ID NO: 4 | 1844 |
| IFD106 | CDR | PRT | US20190015509; SEQ ID NO: 5 | 1845 |
| IFD107 | CDR | PRT | US20190015509; SEQ ID NO: 3 | 1846 |
| IFD108 | CDR | PRT | U.S. Pat. No. 8,562,996; SEQ ID NO: 75 | 1847 |
| IFD109 | CDR | PRT | U.S. Pat. No. 8,562,996; SEQ ID NO: 82 | 1848 |
| IFD110 | CDR | PRT | U.S. Pat. No. 8,562,996; SEQ ID NO: 3 | 1849 |
| IFD111 | CDR | PRT | U.S. Pat. No. 8,562,996; SEQ ID NO: 87 | 1850 |
| IFD112 | CDR | PRT | U.S. Pat. No. 8,562,996; SEQ ID NO: 73 | 1851 |
| IFD113 | CDR | PRT | U.S. Pat. No. 8,562,996; SEQ ID NO: 80 | 1852 |
| IFD114 | CDR | PRT | U.S. Pat. No. 8,562,996; SEQ ID NO: 2 | 1853 |
| IFD115 | CDR | PRT | U.S. Pat. No. 8,562,996; SEQ ID NO: 84 | 1854 |
| IFD116 | CDR | PRT | U.S. Pat. No. 8,562,996; SEQ ID NO: 81 | 1855 |
| IFD117 | CDR | PRT | U.S. Pat. No. 8,562,996; SEQ ID NO: 74 | 1856 |
| IFD118 | CDR | PRT | U.S. Pat. No. 8,562,996; SEQ ID NO: 90 | 1857 |
| IFD119 | CDR | PRT | U.S. Pat. No. 8,562,996; SEQ ID NO: 1 | 1858 |
| IFD120 | CDR | PRT | U.S. Pat. No. 8,562,996; SEQ ID NO: 4 | 1859 |
| IFD121 | CDR | PRT | U.S. Pat. No. 8,562,996; SEQ ID NO: 76 | 1860 |
| IFD122 | CDR | PRT | U.S. Pat. No. 8,562,996; SEQ ID NO: 6 | 1861 |
| IFD123 | CDR | PRT | U.S. Pat. No. 8,562,996; SEQ ID NO: 77 | 1862 |
| IFD124 | CDR | PRT | U.S. Pat. No. 8,562,996; SEQ ID NO: 91 | 1863 |
| IFD125 | CDR | PRT | U.S. Pat. No. 8,562,996; SEQ ID NO: 83 | 1864 |
| IFD126 | CDR | PRT | U.S. Pat. No. 8,562,996; SEQ ID NO: 5 | 1865 |
| IFD127 | CDR | PRT | U.S. Pat. No. 8,562,996; SEQ ID NO: 89 | 1866 |
| IFD128 | CDR | PRT | U.S. Pat. No. 8,562,996; SEQ ID NO: 88 | 1867 |
| IFD129 | CDR | PRT | US20190031747; SEQ ID NO: 8 | 1868 |
| IFD130 | CDR | PRT | US20190031747; SEQ ID NO: 58 | 1869 |
| IFD131 | CDR | PRT | US20190031747; SEQ ID NO: 120 | 1870 |
| IFD132 | CDR | PRT | US20190031747; SEQ ID NO: 168 | 1871 |
| IFD133 | CDR | PRT | US20190031747; SEQ ID NO: 72 | 1872 |
| IFD134 | CDR | PRT | US20190031747; SEQ ID NO: 184 | 1873 |
| IFD135 | CDR | PRT | US20190031747; SEQ ID NO: 216 | 1874 |
| IFD136 | CDR | PRT | US20190031747; SEQ ID NO: 200 | 1875 |
| IFD137 | CDR | PRT | US20190031747; SEQ ID NO: 88 | 1876 |
| IFD138 | CDR | PRT | US20190031747; SEQ ID NO: 56 | 1877 |
| IFD139 | CDR | PRT | US20190031747; SEQ ID NO: 232 | 1878 |
| IFD140 | CDR | PRT | US20190031747; SEQ ID NO: 104 | 1879 |
| IFD141 | CDR | PRT | US20190031747; SEQ ID NO: 344 | 1880 |
| IFD142 | CDR | PRT | US20190031747; SEQ ID NO: 328 | 1881 |
| IFD143 | CDR | PRT | US20190031747; SEQ ID NO: 296 | 1882 |
| IFD144 | CDR | PRT | US20190031747; SEQ ID NO: 248 | 1883 |
| IFD145 | CDR | PRT | US20190031747; SEQ ID NO: 24 | 1884 |
| IFD146 | CDR | PRT | US20190031747; SEQ ID NO: 40 | 1885 |
| IFD147 | CDR | PRT | US20190031747; SEQ ID NO: 312 | 1886 |
| IFD148 | CDR | PRT | US20190031747; SEQ ID NO: 346 | 1887 |
| IFD149 | CDR | PRT | US20190031747; SEQ ID NO: 186 | 1888 |
| IFD150 | CDR | PRT | US20190031747; SEQ ID NO: 330 | 1889 |
| IFD151 | CDR | PRT | US20190031747; SEQ ID NO: 298 | 1890 |
| IFD152 | CDR | PRT | US20190031747; SEQ ID NO: 42 | 1891 |
| IFD153 | CDR | PRT | US20190031747; SEQ ID NO: 26 | 1892 |
| IFD154 | CDR | PRT | US20190031747; SEQ ID NO: 106 | 1893 |
| IFD155 | CDR | PRT | US20190031747; SEQ ID NO: 10 | 1894 |
| IFD156 | CDR | PRT | US20190031747; SEQ ID NO: 234 | 1895 |
| IFD157 | CDR | PRT | US20190031747; SEQ ID NO: 74 | 1896 |
| IFD158 | CDR | PRT | US20190031747; SEQ ID NO: 218 | 1897 |
| IFD159 | CDR | PRT | US20190031747; SEQ ID NO: 154 | 1898 |
| IFD160 | CDR | PRT | US20190031747; SEQ ID NO: 170 | 1899 |
| IFD161 | CDR | PRT | US20190031747; SEQ ID NO: 250 | 1900 |
| IFD162 | CDR | PRT | US20190031747; SEQ ID NO: 314 | 1901 |
| IFD163 | CDR | PRT | U.S. Pat. No. 7,132,100; SEQ ID NO: 1 | 1902 |
| IFD164 | CDR | PRT | U.S. Pat. No. 7,132,100; SEQ ID NO: 5 | 1903 |
| IFD165 | CDR | PRT | US20190031747; SEQ ID NO: 90 | 1904 |
| IFD166 | CDR | PRT | US20190031747; SEQ ID NO: 202 | 1905 |
| IFD167 | CDR | PRT | US20190031747; SEQ ID NO: 122 | 1906 |
| IFD168 | CDR | PRT | US20190031747; SEQ ID NO: 138 | 1907 |
| IFD169 | CDR | PRT | US20190031747; SEQ ID NO: 6 | 1908 |
| IFD170 | CDR | PRT | U.S. Pat. No. 7,132,100; SEQ ID NO: 6 | 1909 |
| IFD171 | CDR | PRT | US20190031747; SEQ ID NO: 324 | 1910 |
| IFD172 | CDR | PRT | US20190031747; SEQ ID NO: 292 | 1911 |
| IFD173 | CDR | PRT | US20190031747; SEQ ID NO: 164 | 1912 |
| IFD174 | CDR | PRT | US20190031747; SEQ ID NO: 116 | 1913 |
| IFD175 | CDR | PRT | US20190031747; SEQ ID NO: 20 | 1914 |
| IFD176 | CDR | PRT | US20190031747; SEQ ID NO: 148 | 1915 |
| IFD177 | CDR | PRT | US20190031747; SEQ ID NO: 196 | 1916 |
| IFD178 | CDR | PRT | US20190031747; SEQ ID NO: 244 | 1917 |
| IFD179 | CDR | PRT | US20190031747; SEQ ID NO: 100 | 1918 |
| IFD180 | CDR | PRT | US20190031747; SEQ ID NO: 340 | 1919 |
| IFD181 | CDR | PRT | US20190031747; SEQ ID NO: 68 | 1920 |
| IFD182 | CDR | PRT | US20190031747; SEQ ID NO: 212 | 1921 |
| IFD183 | CDR | PRT | US20190031747; SEQ ID NO: 180 | 1922 |
| IFD184 | CDR | PRT | US20190031747; SEQ ID NO: 308 | 1923 |
| IFD185 | CDR | PRT | US20190031747; SEQ ID NO: 84 | 1924 |
| IFD186 | CDR | PRT | US20190031747; SEQ ID NO: 4 | 1925 |
| IFD187 | CDR | PRT | US20190031747; SEQ ID NO: 52 | 1926 |
| IFD188 | CDR | PRT | US20190031747; SEQ ID NO: 150 | 1927 |
| IFD189 | CDR | PRT | US20190031747; SEQ ID NO: 86 | 1928 |
| IFD190 | CDR | PRT | US20190031747; SEQ ID NO: 102 | 1929 |
| IFD191 | CDR | PRT | US20190031747; SEQ ID NO: 166 | 1930 |
| IFD192 | CDR | PRT | US20190031747; SEQ ID NO: 310 | 1931 |
| IFD193 | CDR | PRT | US20190031747; SEQ ID NO: 54 | 1932 |
| IFD194 | CDR | PRT | US20190031747; SEQ ID NO: 294 | 1933 |
| IFD195 | CDR | PRT | US20190031747; SEQ ID NO: 198 | 1934 |
| IFD196 | CDR | PRT | US20190031747; SEQ ID NO: 70 | 1935 |
| IFD197 | CDR | PRT | US20190031747; SEQ ID NO: 230 | 1936 |
| IFD198 | CDR | PRT | US20190031747; SEQ ID NO: 342 | 1937 |
| IFD199 | CDR | PRT | US20190031747; SEQ ID NO: 118 | 1938 |
| IFD200 | CDR | PRT | US20190031747; SEQ ID NO: 22 | 1939 |
| IFD201 | CDR | PRT | US20190031747; SEQ ID NO: 246 | 1940 |
| IFD202 | CDR | PRT | US20190031747; SEQ ID NO: 182 | 1941 |
| IFD203 | CDR | PRT | US20190031747; SEQ ID NO: 214 | 1942 |
| IFD204 | CDR | PRT | U.S. Pat. No. 7,132,100; SEQ ID NO: 3 | 1943 |
| IFD205 | CDR | PRT | US20190031747; SEQ ID NO: 64 | 1944 |
| IFD206 | CDR | PRT | US20190031747; SEQ ID NO: 304 | 1945 |
| IFD207 | CDR | PRT | US20190031747; SEQ ID NO: 32 | 1946 |
| IFD208 | CDR | PRT | US20190031747; SEQ ID NO: 38 | 1947 |
| IFD209 | CDR | PRT | US20190031747; SEQ ID NO: 256 | 1948 |
| IFD210 | CDR | PRT | US20190031747; SEQ ID NO: 160 | 1949 |
| IFD211 | CDR | PRT | US20190031747; SEQ ID NO: 320 | 1950 |
| IFD212 | CDR | PRT | US20190031747; SEQ ID NO: 60 | 1951 |
| IFD213 | CDR | PRT | US20190031747; SEQ ID NO: 188 | 1952 |
| IFD214 | CDR | PRT | US20190031747; SEQ ID NO: 300 | 1953 |
| IFD215 | CDR | PRT | US20190031747; SEQ ID NO: 28 | 1954 |
| IFD216 | CDR | PRT | US20190031747; SEQ ID NO: 236 | 1955 |
| IFD217 | CDR | PRT | US20190031747; SEQ ID NO: 76 | 1956 |
| IFD218 | CDR | PRT | US20190031747; SEQ ID NO: 108 | 1957 |
| IFD219 | CDR | PRT | US20190031747; SEQ ID NO: 252 | 1958 |
| IFD220 | CDR | PRT | US20190031747; SEQ ID NO: 220 | 1959 |
| IFD221 | CDR | PRT | US20190031747; SEQ ID NO: 240 | 1960 |
| IFD222 | CDR | PRT | US20190031747; SEQ ID NO: 96 | 1961 |
| IFD223 | CDR | PRT | US20190031747; SEQ ID NO: 208 | 1962 |
| IFD224 | CDR | PRT | US20190031747; SEQ ID NO: 16 | 1963 |
| IFD225 | CDR | PRT | US20190031747; SEQ ID NO: 112 | 1964 |
| IFD226 | CDR | PRT | US20190031747; SEQ ID NO: 352 | 1965 |
| IFD227 | CDR | PRT | US20190031747; SEQ ID NO: 80 | 1966 |
| IFD228 | CDR | PRT | US20190031747; SEQ ID NO: 128 | 1967 |
| IFD229 | CDR | PRT | US20190031747; SEQ ID NO: 192 | 1968 |
| IFD230 | CDR | PRT | US20190031747; SEQ ID NO: 224 | 1969 |
| IFD231 | CDR | PRT | US20190031747; SEQ ID NO: 204 | 1970 |
| IFD232 | CDR | PRT | US20190031747; SEQ ID NO: 12 | 1971 |
| IFD233 | CDR | PRT | US20190031747; SEQ ID NO: 156 | 1972 |
| IFD234 | CDR | PRT | US20190031747; SEQ ID NO: 316 | 1973 |
| IFD235 | CDR | PRT | US20190031747; SEQ ID NO: 124 | 1974 |
| IFD236 | CDR | PRT | US20190031747; SEQ ID NO: 92 | 1975 |
| IFD237 | CDR | PRT | US20190031747; SEQ ID NO: 140 | 1976 |
| IFD238 | CDR | PRT | US20199031747; SEQ ID NO: 348 | 1977 |
| IFD239 | CDR | PRT | U.S. Pat. No. 7,132,100; SEQ ID NO: 2 | 1978 |
| IFD240 | CDR | PRT | US20190031747; SEQ ID NO: 280 | 1979 |
| IFD241 | CDR | PRT | US20190031747; SEQ ID NO: 136 | 1980 |
| IFD242 | CDR | PRT | US20190031747; SEQ ID NO: 264 | 1981 |
| IFD243 | CDR | PRT | US20190031747; SEQ ID NO: 152 | 1982 |
| IFD244 | CDR | PRT | US20190031747; SEQ ID NO: 134 | 1983 |
| IFD245 | FR | DNA | U.S. Pat. No. 8,562,996; SEQ ID NO: 63 | 1984 |
| IFD246 | FR | DNA | U.S. Pat. No. 8,562,996; SEQ ID NO: 59 | 1985 |
| IFD247 | FR | DNA | U.S. Pat. No. 8,562,996; SEQ ID NO: 72 | 1986 |
| IFD248 | FR | DNA | U.S. Pat. No. 8,562,996; SEQ ID NO: 66 | 1987 |
| IFD249 | FR | DNA | U.S. Pat. No. 8,562,996; SEQ ID NO: 70 | 1988 |
| IFD250 | FR | DNA | U.S. Pat. No. 8,562,996; SEQ ID NO: 61 | 1989 |
| IFD251 | FR | DNA | U.S. Pat. No. 8,562,996; SEQ ID NO: 65 | 1990 |
| IFD252 | FR | DNA | U.S. Pat. No. 8,562,996; SEQ ID NO: 68 | 1991 |
| IFD253 | Full antibody | DNA | U.S. Pat. No. 8,562,996; SEQ ID NO: 51 | 1992 |
| IFD254 | Full antibody | PRT | U.S. Pat. No. 8,562,996; SEQ ID NO: 58 | 1993 |
| IFD255 | Full antibody | PRT | U.S. Pat. No. 8,562,996; SEQ ID NO: 57 | 1994 |
| IFD256 | Full antibody | PRT | U.S. Pat. No. 8,562,996; SEQ ID NO: 56 | 1995 |
| IFD257 | HC | DNA | U.S. Pat. No. 8,562,996; SEQ ID NO: 126 | 1996 |
| IFD258 | HC | DNA | U.S. Pat. No. 8,562,996; SEQ ID NO: 111 | 1997 |
| IFD259 | HC | DNA | U.S. Pat. No. 8,562,996; SEQ ID NO: 112 | 1998 |
| IFD260 | HC | DNA | U.S. Pat. No. 8,562,996; SEQ ID NO: 124 | 1999 |
| IFD261 | HC | DNA | U.S. Pat. No. 8,562,996; SEQ ID NO: 9 | 2000 |
| IFD262 | HC | DNA | U.S. Pat. No. 8,562,996; SEQ ID NO: 109 | 2001 |
| IFD263 | HC | DNA | U.S. Pat. No. 8,562,996; SEQ ID NO: 116 | 2002 |
| IFD264 | HC | DNA | U.S. Pat. No. 8,562,996; SEQ ID NO: 131 | 2003 |
| IFD265 | HC | DNA | U.S. Pat. No. 8,562,996; SEQ ID NO: 99 | 2004 |
| IFD266 | HC | DNA | U.S. Pat. No. 8,562,996; SEQ ID NO: 114 | 2005 |
| IFD267 | HC | DNA | U.S. Pat. No. 8,562,996; SEQ ID NO: 125 | 2006 |
| IFD268 | HC | DNA | U.S. Pat. No. 8,562,996; SEQ ID NO: 110 | 2007 |
| IFD269 | HC | DNA | U.S. Pat. No. 8,562,996; SEQ ID NO: 129 | 2008 |
| IFD270 | HC | DNA | U.S. Pat. No. 8,562,996; SEQ ID NO: 127 | 2009 |
| IFD271 | HC | DNA | U.S. Pat. No. 8,562,996; SEQ ID NO: 113 | 2010 |
| IFD272 | HC | DNA | U.S. Pat. No. 8,562,996; SEQ ID NO: 128 | 2011 |
| IFD273 | HC | DNA | U.S. Pat. No. 8,562,996; SEQ ID NO: 98 | 2012 |
| IFD274 | HC | DNA | U.S. Pat. No. 8,562,996; SEQ ID NO: 100 | 2013 |
| IFD275 | HC | DNA | U.S. Pat. No. 8,562,996; SEQ ID NO: 130 | 2014 |
| IFD276 | HC | PRT | U.S. Pat. No. 8,562,996; SEQ ID NO: 92 | 2015 |
| IFD277 | HC | PRT | U.S. Pat. No. 8,562,996; SEQ ID NO: 85 | 2016 |
| IFD278 | HC | PRT | US20190031747; SEQ ID NO: 363 | 2017 |
| IFD279 | HC | PRT | US20190031747; SEQ ID NO: 357 | 2018 |
| IFD280 | HC | PRT | US20190031747; SEQ ID NO: 359 | 2019 |
| IFD281 | HC | PRT | US20190031747; SEQ ID NO: 361 | 2020 |
| IFD282 | LC | DNA | U.S. Pat. No. 8,562,996; SEQ ID NO: 133 | 2021 |
| IFD283 | LC | DNA | U.S. Pat. No. 8,562,996; SEQ ID NO: 120 | 2022 |
| IFD284 | LC | DNA | U.S. Pat. No. 8,562,996; SEQ ID NO: 105 | 2023 |
| IFD285 | LC | DNA | U.S. Pat. No. 8,562,996; SEQ ID NO: 118 | 2024 |
| IFD286 | LC | DNA | U.S. Pat. No. 8,562,996; SEQ ID NO: 106 | 2025 |
| IFD287 | LC | DNA | U.S. Pat. No. 8,562,996; SEQ ID NO: 121 | 2026 |
| IFD288 | LC | DNA | U.S. Pat. No. 8,562,996; SEQ ID NO: 103 | 2027 |
| IFD289 | LC | DNA | U.S. Pat. No. 8,562,996; SEQ ID NO: 136 | 2028 |
| IFD290 | LC | DNA | U.S. Pat. No. 8,562,996; SEQ ID NO: 117 | 2029 |
| IFD291 | LC | DNA | U.S. Pat. No. 8,562,996; SEQ ID NO: 123 | 2030 |
| IFD292 | LC | DNA | U.S. Pat. No. 8,562,996; SEQ ID NO: 10 | 2031 |
| IFD293 | LC | DNA | U.S. Pat. No. 8,562,996; SEQ ID NO: 102 | 2032 |
| IFD294 | LC | DNA | U.S. Pat. No. 8,562,996; SEQ ID NO: 135 | 2033 |
| IFD295 | LC | DNA | U.S. Pat. No. 8,562,996; SEQ ID NO: 119 | 2034 |
| IFD296 | LC | DNA | U.S. Pat. No. 8,562,996; SEQ ID NO: 134 | 2035 |
| IFD297 | LC | DNA | U.S. Pat. No. 8,562,996; SEQ ID NO: 122 | 2036 |
| IFD298 | LC | DNA | U.S. Pat. No. 8,562,996; SEQ ID NO: 132 | 2037 |
| IFD299 | LC | DNA | U.S. Pat. No. 8,562,996; SEQ ID NO: 138 | 2038 |
| IFD300 | LC | DNA | U.S. Pat. No. 8,562,996; SEQ ID NO: 104 | 2039 |
| IFD301 | LC | DNA | U.S. Pat. No. 8,562,996; SEQ ID NO: 107 | 2040 |
| IFD302 | LC | DNA | U.S. Pat. No. 8,562,996; SEQ ID NO: 137 | 2041 |
| IFD303 | LC | PRT | U.S. Pat. No. 8,562,996; SEQ ID NO: 86 | 2042 |
| IFD304 | LC | PRT | U.S. Pat. No. 8,562,996; SEQ ID NO: 93 | 2043 |
| IFD305 | LC | PRT | US20190031747; SEQ ID NO: 362 | 2044 |
| IFD306 | LC | PRT | US20190031747; SEQ ID NO: 360 | 2045 |
| IFD307 | LC | PRT | US20190031747; SEQ ID NO: 358 | 2046 |
| IFD308 | LC | PRT | US20190031747; SEQ ID NO: 364 | 2047 |
| IFD309 | VH | DNA | US20190015509; SEQ ID NO: 1 | 2048 |
| IFD310 | VH | DNA | U.S. Pat. No. 8,562,996; SEQ ID NO: 96 | 2049 |
| IFD311 | VH | DNA | U.S. Pat. No. 8,562,996; SEQ ID NO: 94 | 2050 |
| IFD312 | VH | DNA | U.S. Pat. No. 8,562,996; SEQ ID NO: 101 | 2051 |
| IFD313 | VH | DNA | US20190031747; SEQ ID NO: 305 | 2052 |
| IFD314 | VH | DNA | US20190031747; SEQ ID NO: 177 | 2053 |
| IFD315 | VH | DNA | US20190031747; SEQ ID NO: 209 | 2054 |
| IFD316 | VH | DNA | US20190031747; SEQ ID NO: 289 | 2055 |
| IFD317 | VH | DNA | US20190031747; SEQ ID NO: 49 | 2056 |
| IFD318 | VH | DNA | US20190031747; SEQ ID NO: 1 | 2057 |
| IFD319 | VH | DNA | US20190031747; SEQ ID NO: 81 | 2058 |
| IFD320 | VH | DNA | US20190031747; SEQ ID NO: 33 | 2059 |
| IFD321 | VH | DNA | US20190031747; SEQ ID NO: 17 | 2060 |
| IFD322 | VH | DNA | US20190031747; SEQ ID NO: 241 | 2061 |
| IFD323 | VH | DNA | US20190031747; SEQ ID NO: 225 | 2062 |
| IFD324 | VH | DNA | US20190031747; SEQ ID NO: 193 | 2063 |
| IFD325 | VH | DNA | US20190031747; SEQ ID NO: 321 | 2064 |
| IFD326 | VH | DNA | US20190031747; SEQ ID NO: 129 | 2065 |
| IFD327 | VH | DNA | US20190031747; SEQ ID NO: 257 | 2066 |
| IFD328 | VH | DNA | US20190031747; SEQ ID NO: 273 | 2067 |
| IFD329 | VH | DNA | US20190031747; SEQ ID NO: 113 | 2068 |
| IFD330 | VH | DNA | US20190031747; SEQ ID NO: 161 | 2069 |
| IFD331 | VH | DNA | US20190031747; SEQ ID NO: 65 | 2070 |
| IFD332 | VH | DNA | US20190031747; SEQ ID NO: 337 | 2071 |
| IFD333 | VH | DNA | US20190031747; SEQ ID NO: 145 | 2072 |
| IFD334 | VH | DNA | US20190031747; SEQ ID NO: 97 | 2073 |
| IFD335 | VH | PRT | US20190015509; SEQ ID NO: 2 | 2074 |
| IFD336 | VH | PRT | U.S. Pat. No. 8,562,996; SEQ ID NO: 78 | 2075 |
| IFD337 | VH | PRT | U.S. Pat. No. 8,562,996; SEQ ID NO: 55 | 2076 |
| IFD338 | VH | PRT | US20190031747; SEQ ID NO: 274 | 2077 |
| IFD339 | VH | PRT | US20190031747; SEQ ID NO: 130 | 2078 |
| IFD340 | VH | PRT | US20190031747; SEQ ID NO: 258 | 2079 |
| IFD341 | VH | PRT | US20190031747; SEQ ID NO: 162 | 2080 |
| IFD342 | VH | PRT | US20190031747; SEQ ID NO: 66 | 2081 |
| IFD343 | VH | PRT | US20190031747; SEQ ID NO: 338 | 2082 |
| IFD344 | VH | PRT | US20190031747; SEQ ID NO: 98 | 2083 |
| IFD345 | VH | PRT | US20190031747; SEQ ID NO: 146 | 2084 |
| IFD346 | VH | PRT | US20190031747; SEQ ID NO: 114 | 2085 |
| IFD347 | VH | PRT | US20190031747; SEQ ID NO: 290 | 2086 |
| IFD348 | VH | PRT | US20190031747; SEQ ID NO: 322 | 2087 |
| IFD349 | VH | PRT | US20190031747; SEQ ID NO: 210 | 2088 |
| IFD350 | VH | PRT | US20190031747; SEQ ID NO: 178 | 2089 |
| IFD351 | VH | PRT | US20190031747; SEQ ID NO: 306 | 2090 |
| IFD352 | VH | PRT | US20190031747; SEQ ID NO: 194 | 2091 |
| IFD353 | VH | PRT | US20190031747; SEQ ID NO: 226 | 2092 |
| IFD354 | VH | PRT | US20190031747; SEQ ID NO: 18 | 2093 |
| IFD355 | VH | PRT | US20190031747; SEQ ID NO: 242 | 2094 |
| IFD356 | VH | PRT | US20190031747; SEQ ID NO: 34 | 2095 |
| IFD357 | VH | PRT | US20190031747; SEQ ID NO: 50 | 2096 |
| IFD358 | VH | PRT | US20190031747; SEQ ID NO: 2 | 2097 |
| IFD359 | VH | PRT | US20190031747; SEQ ID NO: 82 | 2098 |
| IFD360 | VL | DNA | US20190015509; SEQ ID NO: 6 | 2099 |
| IFD361 | VL | DNA | U.S. Pat. No. 8,562,996; SEQ ID NO: 108 | 2100 |
| IFD362 | VL | DNA | US20190031747; SEQ ID NO: 121 | 2101 |
| IFD363 | VL | DNA | US20190031747; SEQ ID NO: 137 | 2102 |
| IFD364 | VL | DNA | US20190031747; SEQ ID NO: 201 | 2103 |
| IFD365 | VL | DNA | US20190031747; SEQ ID NO: 89 | 2104 |
| IFD366 | VL | DNA | US20190031747; SEQ ID NO: 185 | 2105 |
| IFD367 | VL | DNA | US20190031747; SEQ ID NO: 345 | 2106 |
| IFD368 | VL | DNA | US20190031747; SEQ ID NO: 9 | 2107 |
| IFD369 | VL | DNA | US20190031747; SEQ ID NO: 105 | 2108 |
| IFD370 | VL | DNA | US20190031747; SEQ ID NO: 297 | 2109 |
| IFD371 | VL | DNA | US20190031747; SEQ ID NO: 41 | 2110 |
| IFD372 | VL | DNA | US20190031747; SEQ ID NO: 25 | 2111 |
| IFD373 | VL | DNA | US20190031747; SEQ ID NO: 329 | 2112 |
| IFD374 | VL | DNA | US20190031747; SEQ ID NO: 73 | 2113 |
| IFD375 | VL | DNA | US20190031747; SEQ ID NO: 233 | 2114 |
| IFD376 | VL | DNA | US20190031747; SEQ ID NO: 217 | 2115 |
| IFD377 | VL | DNA | US20190031747; SEQ ID NO: 313 | 2116 |
| IFD378 | VL | DNA | US20190031747; SEQ ID NO: 153 | 2117 |
| IFD379 | VL | DNA | US20190031747; SEQ ID NO: 169 | 2118 |
| IFD380 | VL | DNA | US20190031747; SEQ ID NO: 249 | 2119 |
| IFD381 | VL | DNA | US20190031747; SEQ ID NO: 57 | 2120 |
| IFD382 | VL | PRT | US20190015509; SEQ ID NO: 7 | 2121 |
| IFD383 | VL | PRT | U.S. Pat. No. 8,562,996; SEQ ID NO: 79 | 2122 |
| IFD384 | LC | PRT | U.S. Pat. No. 7,122,185; SEQ ID NO: 6 | 2123 |
| IFD385 | LC | PRT | WO2018104893; SEQ ID NO: 9 | 2124 |
| IFD386 | HC | PRT | U.S. Pat. No. 7,122,185; SEQ ID NO: 9 | 2125 |
| IFD387 | HC | PRT | U.S. Pat. No. 7,122,185; SEQ ID NO: 12 | 2126 |
| IFD388 | HC | PRT | WO2018104893; SEQ ID NO: 12 | 2127 |
| IFD389 | HC | PRT | WO2018104893; SEQ ID NO: 13 | 2128 |
| IFD390 | HC | PRT | WO2018104893; SEQ ID NO: 14 | 2129 |
| IFD391 | VL | PRT | WO2018104893; SEQ ID NO: 7 | 2130 |
| IFD392 | VH | PRT | WO2018104893; SEQ ID NO: 8 | 2131 |
| IFD393 | scFv | PRT | WO2017053556; SEQ ID NO: 13 | 2132 |
| IFD394 | scFv | PRT | WO2017053556; SEQ ID NO: 17 | 2133 |
| IFD395 | scFv | PRT | WO2017053556; SEQ ID NO: 21 | 2134 |
| IFD396 | scFv | PRT | WO2017053556; SEQ ID NO: 25 | 2135 |
| IFD397 | scFv | PRT | WO2017053556; SEQ ID NO: 29 | 2136 |
| IFD398 | scFv | PRT | WO2017053556; SEQ ID NO: 57 | 2137 |
| IFD399 | scFv | PRT | WO2017053556; SEQ ID NO: 58 | 2138 |
| IFD400 | scFv | PRT | WO2017053556; SEQ ID NO: 59 | 2139 |
| IFD401 | scFv | PRT | WO2017053556; SEQ ID NO: 69 | 2140 |
| IFD402 | scFv | PRT | WO2017053556; SEQ ID NO: 61 | 2141 |

In some embodiments, the payload region of the AAV particle comprises a nucleic acid sequence encoding a polypeptide which is an antibody, an antibody-based composition, or a fragment thereof. As anon-limiting example, the antibody may be one or more of the polypeptides listed in Table 3, or variants or fragments thereof. As another non-limiting example, the antibody may be one or more of the heavy chain sequences listed in Table 3. As a non-limiting example, the antibody may be one or more of the light chain sequences listed in Table 3, or variants or fragments thereof.

In some embodiments, the payload region of the AAV particle comprises a nucleic acid sequence encoding a polypeptide comprising a heavy chain and alight chain sequence listed in Table 3, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and a light chain sequence listed in Table 3, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 3, one or more linkers from Table 2 and a heavy chain sequence from Table 3.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 3, one or more linkers from Table 2, and alight chain sequence from Table 3.

In some embodiments, the payload region comprises a nucleic acid sequence encoding a single heavy chain. As a non-limiting example, the heavy chain is an amino acid sequence or fragment thereof described in Table 3.

Shown in Table 3 are a listing of antibodies and their polynucleotides and/or polypeptides sequences. These sequences may be encoded by or included in the AAV particles of the present disclosure. Variants or fragments of the antibody sequences described in Table 3 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 3. CDRs of the antibodies encoded by the viral genomes of the present disclosure may be 50%, 60%, 70%, 80%, 90%, 95% identical to CDRs listed in or incorporated in the sequences of Table 3. Methods of determining CDRs are well known in the art and are described herein. Payload regions may encode antibody variants with one or more heavy chain variable domain (V_{H}) or light chain variable domain (V₄) derived from the antibody sequences in Table 3. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al (J. of. Virology, September 2002, Vol. 76(17), p 8769-8775; the contents of which are herein incorporated by reference in their entirety).

Payload regions of the viral genomes may encode any infectious disease-associated antibody, not limited to those described in Table 3, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the infectious disease-associated antibodies as described in International Publication Number WO2016059622, WO2017046658, WO2017046676, WO2017048593, WO2017048614, WO2017048902, WO2017049035, WO2017049231, WO2017049266, WO2017052679, WO2017053170, WO2017053703, WO2017053807, WO2017053889, WO2017055273, WO2017055404, WO2017055617, WO2017058115, WO2017058866, WO2017059095, WO2017059289, WO2017059551, WO2017059878, WO2017060247, WO2017060504, WO2017060857, WO2017061599, WO2017062820, WO2017062888, WO2017062952, WO2017063593, WO2017064221, WO2017068186, WO2017070364, WO2017070395, WO2017070423, WO2017070476, WO2017070613, WO2017070616, WO2017070622, WO2017070623, WO2017070624, WO2017070626, WO2017070654, WO2017073981, WO2017074074, WO2017075052, WO2017075119, WO2017075124, WO2017075188, WO2017075432, WO2017075533, WO2017075540, WO2017075615, WO2017076308, WO2017077047, WO2017078839, WO2017079112, WO2017079115, WO2017079116, WO2017079369, WO2017079443, WO2017079479, WO2017079681, WO2017079705, WO2017081066, WO2017082214, WO2017083515, WO2017083627, WO2017083750, WO2017083762, WO2017083764, WO2017084495, WO2017085212, WO2017086627, WO2017087587, WO2017087588, WO2017087589, WO2017087599, WO2017087678, WO2017087921, WO2017088269, WO2017089895, WO2017091487, WO2017092645, WO2017093448, WO2017093845, WO2017093985, WO2017095823, WO2017095875, WO2017096179, WO2017096182, WO2017096189, WO2017096221, WO2017096276, WO2017096281, WO2017096327, WO2017096329, WO2017099712, WO2017100289, WO2017102010, WO2017103088, WO2017106061, WO2017106129, WO2017106236, WO2017106326, WO2017106346, WO2017106656, WO2017109721, WO2017112536, WO2017112877, WO2017112944, WO2017114694, WO2017118321, WO2017118745, WO2017118761, WO2017120222, WO2017120280, WO2017120344, WO2017120525, WO2017120599, WO2017120996, WO2017120997, WO2017120998, WO2017123160, WO2017123556, WO2017123557, WO2017123644, WO2017123646, WO2017123685, WO2017123978, WO2017125487, WO2017125578, WO2017125871, WO2017127468, WO2017127764, WO2017128534, WO2017129064, WO2017130223, WO2017132562, WO2017133174, WO2017133175, WO2017133633, WO2017133639, WO2017133640, WO2017134140, WO2017134440, WO2017135791, WO2017136607, WO2017137542, WO2017137583, WO2017137954, WO2017139276, WO2017139587, WO2017139975, WO2017140256, WO2017143062, WO2017143270, WO2017144621, WO2017144668, WO2017144896, WO2017147248, WO2017147368, WO2017147383, WO2017147538, WO2017147719, WO2017148889, WO2017149143, WO2017149515, WO2017149538, WO2017151176, WO2017151818, WO2017151940, WO2017152076, WO2017152088, WO2017153402, WO2017153433, WO2017153438, WO2017156355, WO2017156423, WO2017156479, WO2017156500, WO2017158337, WO2017158339, WO2017159996, WO2017161206, WO2017162678, WO2017164678, WO2017165245, WO2017165398, WO2017165460, WO2017165464, WO2017165683, WO2017165734, WO2017165736, WO2017174568, WO2017174586, WO2017175176, WO2017176007, WO2017177013, WO2017177137, WO2017177175, WO2017177199, WO2017177217, WO2017178653, WO2017180536, WO2017180738, WO2017180936, WO2017180976, WO2017180993, WO2017181011, WO2017181015, WO2017181031, WO2017181039, WO2017181098, WO2017181109, WO2017181119, WO2017181420, WO2017182672, WO2017183711, WO2017186121, WO2017186784, WO2017186928, WO2017187307, WO2017188570, WO2017189432, WO2017189959, WO2017189963, WO2017189964, WO2017190100, WO2017191062, WO2017192483, WO2017192567, WO2017192589, WO2017192933, WO2017192946, WO2017193101, WO2017193107, WO2017194265, WO2017194555, WO2017194783, WO2017196819, WO2017196847, WO2017197331, WO2017197347, WO2017197376, WO2017197667, WO2017198212, WO2017199094, WO2017199250, WO2017201131, WO2017201210, WO2017201488, WO2017201493, WO2017201731, WO2017201766, WO2017202387, WO2017205014, WO2017205631, WO2017205694, WO2017205721, WO2017205726, WO2017205820, WO217205875, WO2017206621, WO2017207477, WO2017207480, WO2017207775, WO2017207791, WO2017210058, WO2017211278, WO2017211900, WO2017214298, WO2017215590, WO2017218355, WO2017218435, WO2017218515, WO2017218698, WO2017218977, WO2017219025, WO2017219029, WO2017220800, WO2017220988, WO2017220989, WO2017220990, WO2017222556, WO2017223283, WO2017223286, WO2018002339, WO2018002952, WO2018005556, WO2018005558, WO2018005559, WO2018006005, WO2018006092, WO2018006824, WO2018006882, WO2018009894, WO2018010789, WO2018011283, WO2018011421, WO2018011573, WO2018011584, WO2018011799, WO2018012952, WO2018013585, WO2018013818, WO2018013918, WO2018017497, WO2018017708, WO2018017797, WO2018017864, WO2018017964, WO2018017996, WO2018018039, WO2018019897, WO2018020476, WO2018022479, WO2018022608, WO2018022786, WO2018023093, WO2018023100, WO2018023136, WO2018025178, WO201802018, WO2018026249, WO2018026600, WO2018026715, WO2018026742, WO2018026953, WO2018026969, WO2018026992, WO2018027039, WO2018027042, WO2018027155, WO2018027185, WO2018027252, WO2018028635, WO2018029284, WO2018030777, WO2018031490, WO2018034225, WO2018034226, WO2018034227, WO2018035001, WO2018035046, WO2018035084, WO2018035311, WO2018035407, WO2018035710, WO2018036561 WO2018038096, WO2018038945, WO2018039097, WO2018039514, WO2018039626, WO2018042385, WO2018044619, WO2018044970, WO2018045018, WO2018045325, WO2018048318, WO2018048939, WO2018049124, WO2018049130, WO2018049188, WO2018049248, WO2018050783, WO2018050852, WO2018051348, WO2018052375, WO2018052789, WO2018053029, WO2018053180, WO2018053270, WO2018053434, WO2018054240, WO2018054241, WO2018056821, WO2018056897, WO2018057585, WO2018057735, WO2018057776, WO2018057823, WO2018057904, WO2018057916, WO2018057967, WO2018058002 WO2018058022, WO2018058177, WO2018059117, WO2018064190, WO2018064478, WO2018064602, WO2018064611, WO2018065552, WO2018067580, WO2018067582, WO2018067618, WO2018067991, WO2018067993, WO2018068354, WO2018069279, WO2018071345, WO2018071777, WO2018071796, WO2018071822, WO2018071873, WO2018073387, WO2018075378, WO2018075564, WO2018075591, WO2018075621, WO2018075794, WO2018075813, WO2018075820, WO2018075954, WO2018075961, WO2018075974, WO2018075980, WO2018075989, WO2018077208, WO2018077242, WO2018077893, WO2018077926, WO2018081282, WO2018081329, WO2018081590, WO2018081642, WO2018081649, WO2018081754, WO2018081755, WO2018081832, WO2018083087, WO2018083692, WO2018085358, WO2018085400, WO2018085468, WO2018085469, WO2018085731, WO2018085815, WO2018085842, WO2018086139, WO2018086599, WO2018089293, WO2018089335, WO2018089829, WO2018090057, WO2018091740, WO2018094300, WO2018094414, WO2018098354, WO2018098362, WO2018098365, WO2018098480, WO2018099968, WO2018102589, WO2018102597, WO2018102612, WO2018102746, WO2018102785, WO2018102795, WO2018103501, WO2018103502, WO2018103503, WO2018104407, WO2018104528, WO2018104556, WO2018106712, WO2018106732, WO2018106862, WO2018106864, WO2018108106, WO2018109663, WO2018111852, WO2018112426, WO2018112549, WO2018113258, WO2018115262, WO2018115485, WO2018115885, WO2018115887, WO2018118754, WO2018118780, WO2018119001, WO2018119114, WO2018119171, WO2018119474, WO2018119475, WO2018121679, WO2018124121, WO2018126232, WO2018126287, WO2018127473, WO2018127709, WO2018127710, WO2018127711, WO2018127713, WO2018127787, WO2018129400, WO2018129404, WO2018129474, WO2018129524, WO2018130657, WO2018132423, WO2018132739, WO2018133837, WO2018133842, WO2018133877, WO2018134389, WO2018134691, WO2018134787, WO2018134817, WO2018136163, WO2018136626, WO2018136823, WO2018137293, WO2018137294, WO2018137295, WO2018137576, WO2018138297, WO2018138496, WO2018138681, WO2018140242, WO2018140660, WO2018140725, WO2018140733, WO2018140845, WO2018140970, WO2018140973, WO2018141910, WO2018145125, WO2018146317, WO2018146549, WO2018148180, WO2018148223, WO2018148224, WO2018148383, WO2018149358, WO2018150029, WO2018150345, WO2018151816, WO2018152197, WO2018152452, WO2018152687, WO2018153340, WO2018154392, WO2018156250, WO2018156367, WO2018156654, WO2018156735, WO2018156791, WO2018158632, WO2018158658, WO2018159845, WO2018160731, WO2018161017, WO2018161092, WO2018161872, WO2018162944, WO2018165089, WO2018165228, WO2018165475, WO2018167322, WO2018169785, WO2018169922, WO2018169948, WO2018169953, WO2018169993, WO2018170134, WO2018170145, WO2018170188, WO2018170256, WO2018170338, WO2018170408, WO2018172957, WO2018175460, WO2018175476, WO2018175531, WO2018175752, WO2018175788, WO2018175917, WO2018175988, WO2018176505, WO2018176844, WO2018178047, WO2018178122, WO2018178123, WO2018181064, WO2018181866, WO2018183219, WO2018183293, WO2018183459, WO2018185043, WO2018185110, WO2018185526, WO2018185709, WO2018187190, WO2018187191, WO2018187332, WO2018187613, WO2018187706, WO2018187791, WO2018187799, WO2018188331, WO2018189225, WO2018189382, WO2018190719, WO2018191313, WO2018191502, WO2018191545, WO2018191723, WO2018192365, WO2018193063, WO2018193457, WO2018201096, WO2018201099, WO2018204363, WO2018204427, WO2018204520, WO2018204677, WO2018208231, WO2018208856, WO2018208868, WO2018209194, WO2018210898, WO2018213097, WO2018213204, WO2018213316, WO2018213592, WO2018215535, WO2018215571, WO2018215831, WO2018215935, WO2018215936, WO2018215937, WO2018215938, WO2018217681, WO2018217688, WO2018217940, WO2018218083, WO2018218185, WO2018218215, WO2018218219, WO2018218222, WO2018218240, WO2018218875, WO2018218876, WO2018218877, WO2018218878, WO2018218879, WO2018220100, WO2018220216, WO2018220584, WO2018221521, WO2018221892, WO2018222019, WO2018222139, WO2018222718, WO2018222722, WO2018223600, WO2018223601, WO2018224609, WO2018224611, WO2018224630, WO2018226992, WO2018229193, WO2018229194, WO2018229195, WO2018229197, WO2018229492, WO2018229530, WO2018229612, WO2018232088, WO2018232355, WO2018232372, WO2018232467, WO2018233333, WO2018233813, WO2018234576, WO2018234793, WO2018237010, WO2018237148, WO2018237192, WO2018237287, WO2018237326, WO2018237357, WO2019000105, WO2019000620, WO2019001417, WO2019003074, WO2019003164, WO2019004136, WO2019004831, WO2019006007, WO2019006043, WO2019009726, WO2019009727, WO2019009728, WO2019010486, WO2019011306, WO2019011855, WO2019012138, WO2019012141, WO2019012336, WO2019014405, WO2019014623, WO2019015673, WO2019016247, WO2019016784, WO2019018310, WO2019018629, WO2019018640, WO2019018730, WO2019023347, WO2019023396, WO2019023410, WO2019023482, WO2019023504, WO2019025865, WO2019027903, WO2019027935, WO2019028051, WO2019028182, WO2019028456, WO2019028530, WO2019028980, WO2019030377, WO2019031938, WO2019031939, WO2019032699, WO2019032898, WO2019032916, WO2019032927, WO2019032929, WO2019033057, WO2019033087, WO2019034177, WO2019036688, WO2019036725, WO2019036842, WO2019036855, WO2019036856, WO2019037711, WO2019038368, WO2019040617, WO2019040649, WO2019042153, WO2019042555, WO2019043166, WO2019044926, WO2019046225, WO2019046856, WO2019047932, WO2019050326, WO2019051127, WO2019051128, WO2019051132, WO2019051335, WO2019051470, WO2019051586, WO2019052562, WO2019053420, WO2019055842, WO2019057099, WO2019057102, WO2019057122, WO2019057772, WO2019057992, WO2019060619, WO2019062832, WO2019066535, WO2019066536, WO2019067805, WO2019067815, WO2019070435, WO2019070541, WO2019070655, WO2019070740, WO2019073058, WO2019073080, WO2019075300, WO2019075385, WO2019075433, WO2019075487, WO2019076277, WO2019076486, WO2019078591, WO2019078600, WO2019078916, WO2019079240, WO2019079249, WO2019079337, WO2019079569, WO2019079671, WO2019079772, WO2019080858, WO2019081022, WO2019081595, WO2019081692, WO2019082020, WO2019082208, WO2019083506, WO2019084018, WO2019084057, WO2019084064, WO2019084067, WO2019084552, WO2019085102, WO2019085238, WO2019086500, WO2019086512, WO2019087087, WO2019088658, WO2019089610, WO2019089755, WO2019089855, WO2019089921, WO2019089969, WO2019089982, WO2019090003, WO2019090004, WO2019090069, WO2019090074, WO2019090076, WO2019090078, WO2019090081, WO2019090082, WO2019090085, WO2019090088, WO2019090090, WO2019090110, WO2019090355, WO2019092181, WO2019092451, WO2019092452, WO2019092505, WO2019092507, WO2019092677, WO2019094095, WO2019094482, WO2019094700, WO2019094983, WO2019096136, WO2019097305, WO2019099433, WO2019099454, WO2016124768, WO2019057755, WO2018222741, WO2018209265, WO2018160722, WO2018147018, WO2018136775, WO2018136774, WO2018125813, WO2018098553, WO2018085801, WO2018034298, WO2018023976, WO2018002902, WO2017217744, WO2017205377, WO2017139153, WO2017125892, WO2017116212, WO2017074878, WO2017070603, WO2017070594, WO2017059813, and WO2017053482, the contents of each of which are herein incorporated by reference in their entirety.

In some embodiments, payloads may encode infectious disease-associated antibodies (or fragments thereof) taught in U.S. Pat. No. 8,562,996, the contents of which are herein incorporated by reference in their entirety. Such embodiments may include antibody AM14 or fragments thereof. In certain embodiments, the payload region encodes antibody AM14 or fragments thereof selected from SEQ ID NO: 78-79, 101, 108 as described in U.S. Pat. No. 8,562,996.

In some embodiments, payloads may encode infectious disease-associated antibodies (or fragments thereof) taught in U.S. Pat. No. 8,562,996, the contents of which are herein incorporated by reference in their entirety. Such embodiments may include antibody AM16 or fragments thereof. In certain embodiments, the payload region encodes antibody AM16 or fragments thereof selected from SEQ ID NO: 85-86, 116, 123 as described in U.S. Pat. No. 8,562,996.

In some embodiments, payloads may encode infectious disease-associated antibodies (or fragments thereof) taught in U.S. Pat. No. 8,562,996, the contents of which are herein incorporated by reference in their entirety. Such embodiments may include antibody AM23 or fragments thereof. In certain embodiments, the payload region encodes antibody AM23 or fragments thereof selected from SEQ ID NO: 92-93, 131, 138 as described in U.S. Pat. No. 8,562,996.

In some embodiments, payloads may encode infectious disease-associated antibodies (or fragments thereof) taught in US Patent Publication Number U520190031747, the contents of which are herein incorporated by reference in their entirety. Such embodiments may include antibodies AM22 or fragments thereof. In one embodiment, the payload region encodes antibody AM22 or fragments thereof selected from SEQ ID NO: 357-358 as described in US20190031747

In some embodiments, payloads may encode infectious disease-associated antibodies (or fragments thereof) taught in U.S. Pat. No. 8,562,996, the contents of which are herein incorporated by reference in their entirety. Such embodiments may include antibodies MEDI8897 or fragments thereof. In certain embodiments, the payload region encodes antibody MEDI8897 or fragments thereof selected from SEQ ID NO: 59-72 as described in U.S. Pat. No. 8,562,996.

In some embodiments, payloads may encode infectious disease-associated antibodies (or fragments thereof) taught in US Patent Publication Number US20190031747, the contents of which are herein incorporated by reference in their entirety. Such embodiments may include antibody REGN222 or fragments thereof. In certain embodiments, the payload region encodes antibody REGN222 or fragments thereof selected from SEQ ID NO: 1.315 and 363-364 as described in US20190031747.

In some embodiments, payloads may encode infectious disease-associated antibodies (or fragments thereof) taught in US Patent Publication Number US20190015509, the contents of which are herein incorporated by reference in their entirety. Such embodiments may include antibody MEDI8852 or fragments thereof. In certain embodiments, the payload region encodes antibody MEDI8852 or fragments thereof selected from SEQ ID NO: 1-10 as described in US20190015509.

In some embodiments, payloads may encode infectious disease-associated antibodies (or fragments thereof) taught in US Patent Publication Number US20190031747, the contents of which are herein incorporated by reference in their entirety. Such embodiments may include antibody Palivizumab or SYNAGIS, or fragments thereof. In certain embodiments, the payload region encodes antibody Palivizumab or SYNAGIS or fragments thereof selected from SEQ ID NO: 361.362 as described in US20190031747.

In some embodiments, payloads may encode infectious disease-associated antibodies (or fragments thereof) taught in U.S. Pat. No. 7,132,100, the contents of which are herein incorporated by reference in their entirety. Such embodiments may include SYNAGIS, or fragments thereof. In certain embodiments, the payload region encodes antibody SYNAGIS or fragments thereof selected from SEQ ID NO:1-6 as described in U.S. Pat. No. 7,132,100.

In some embodiments, payloads may encode infectious disease-associated antibodies (or fragments thereof) taught in US Patent Publication Number U520190031747, the contents of which are herein incorporated by reference in their entirety. Such embodiments may include antibody NUMAX or Motavizumab, or fragments thereof. In certain embodiments, the payload region encodes antibody NUMAX or Motavizumab or fragments thereof selected from SEQ ID NO: 359-360 as described in US20190031747.

In some embodiments, payloads may encode infectious disease-associated antibodies (or fragments thereof) taught in International Publication Number WO2016124768, the contents of which are herein incorporated by reference in their entirety. Such embodiments may include antibody MD3606, or fragments thereof.

In some embodiments, the payload region of the viral particle comprises one or more nucleic acid sequences encoding one or more of the infectious disease related payload antibody polypeptides listed in Tables 32-53 of U.S. provisional patent application 62/844,433, the contents of which are herein incorporated by reference in their entirety.

In some embodiments, the payload region of the viral particle comprises one or more nucleic acid sequences encoding one or more of the payload antibody polypeptides listed in Table 32 of U.S. provisional patent application 62/844,433 against Influenza virus (INFL1-INFL1085; SEQ ID NO: 23496-24580), the contents of which are herein incorporated by reference in their entirety.

In some embodiments, the payload region of the viral particle comprises one or more nucleic acid sequences, fragments or variants thereof or encodes one or more polypeptides, fragments or variants thereof described in U.S. Pat. Nos. 8,003,106 and 8,540,995, International Patent Publication No. WO2015028478, WO2012045001, US Publication No. US20150239960 and US20130251715, the contents of each of which are herein incorporated by reference in their entirety, against influenza.

In some embodiments, the payload region of the viral particle comprises one or more nucleic acid sequences encoding one or more of the payload antibody polypeptides listed in Table 33 of U.S. provisional patent application 62/844,433 against Respiratory Syncytial Virus (RSV1-RSV1088; SEQ ID NO: 24581-25668), the contents of which are herein incorporated by reference in their entirety.

In some embodiments, the payload region of the viral particle comprises one or more nucleic acid sequences, fragments or variants thereof or encodes one or more polypeptides, fragments or variants thereof described in US Publication No. US20140363427, and International Publication No. WO2004083373, the contents of each of which are herein incorporated by reference in their entirety, against RSV F or RSV G protein.

In some embodiments, the payload region of the viral particle comprises one or more nucleic acid sequences encoding one or more of the payload antibody polypeptides listed in Table 34 of U.S. provisional patent application 62/844,433 against Hepatitis B, Hepatitis C and/or Hepatitis D (HEPBD1-HEPBD317; SEQ ID NO: 25669-25985), the contents of which are herein incorporated by reference in their entirety.

In some embodiments, the payload region of the viral particle comprises one or more nucleic acid sequences, fragments or variants thereof or encodes one or more polypeptides, fragments or variants thereof described in U.S. Pat. No. 7,241,445, and U8858947, the contents of each of which are herein incorporated by reference in their entirety, against HCV.

In some embodiments, the payload region of the viral particle comprises one or more nucleic acid sequences, fragments or variants thereof or encodes one or more polypeptides, fragments or variants thereof described in US Publication No. US20150072885 and US20110046354, U.S. Pat. No. 5,204,095, European Publication No. EP0232921, EP0038642, and EP0186371, and International Publication No. WO1994011495, the contents of each of which are herein incorporated by reference in their entirety, against HBV.

In some embodiments, the payload region of the viral particle comprises one or more nucleic acid sequences, fragments or variants thereof or encodes one or more polypeptides, fragments or variants thereof described in U.S. Pat. No. 6,020,195, the contents of which are herein incorporated by reference in their entirety, against HGV (hepatitis G virus).

In some embodiments, the payload region of the viral particle comprises one or more nucleic acid sequences encoding one or more of the payload antibody polypeptides listed in Table 35 of U.S. provisional patent application 62/844,433 against Herpes Virus (HERP1-HERP109; SEQ ID NO: 25986-26094), the contents of which are herein incorporated by reference in their entirety.

In some embodiments, the payload region of the viral particle comprises one or more nucleic acid sequences, fragment or variants thereof or encodes one or more polypeptides, fragments or variants thereof described in International Publication No. WO2010109874, and WO1997026329, the contents of each of which are herein incorporated by reference in their entirety, against HSV.

In some embodiments, the payload region of the viral particle comprises one or more nucleic acid sequences, fragment or variants thereof or encodes one or more polypeptides, fragments or variants thereof described in International Publication No. WO1995031546, the contents of which are herein incorporated by reference in their entirety, against VZV.

In some embodiments, the payload region of the viral particle comprises one or more nucleic acid sequences encoding one or more of the payload antibody polypeptides listed in Table 36 of U.S. provisional patent application 62/844,433 against Coronavirus (CORV1-CORV65; SEQ ID NO: 26095-26159), the contents of which are herein incorporated by reference in their entirety.

In some embodiments, the payload region of the viral particle comprises one or more nucleic acid sequences, fragments or variants thereof or encodes one or more polypeptides, fragments or variants thereof described in U.S. Pat. No. 7,629,443, US Publication No. US20080254440, Chinese Publication No. CN103613666, CN1570638, CN101522208, CN1673231, CN1590409, CN1557838, and CN1488645, the contents of each of which are herein incorporated by reference in their entirety, against SARS.

In some embodiments, the payload region of the viral particle comprises one or more nucleic acid sequences encoding one or more of the payload antibody polypeptides listed in Table 37 of U.S. provisional patent application 62/844,433 against John Cunningham Virus (JCV1-JCV68; SEQ ID NO: 26160-26223), the contents of which are herein incorporated by reference in their entirety.

In some embodiments, the payload region of the viral particle comprises one or more nucleic acid sequences encoding one or more of the payload antibody polypeptides listed in Table 38 of U.S. provisional patent application 62/844,433 against Poxvirus (POXV1-POXV11; SEQ ID NO: 26224-26233), the contents of which are herein incorporated by reference in their entirety.

In some embodiments, the payload region of the viral particle comprises one or more nucleic acid sequences encoding one or more of the payload antibody polypeptides listed in Table 39 of U.S. provisional patent application 62/844,433 against Enterovirus 71 (ENTV1-ENTV16; SEQ ID NO: 26234-26249), the contents of which are herein incorporated by reference in their entirety.

In some embodiments, the payload region of the viral particle comprises one or more nucleic acid sequences, fragments or variants thereof or encodes one or more polypeptides, fragments or variants thereof described in Chinese Publication No. CN104357400, the contents of which are herein incorporated by reference in their entirety, against EV71.

In some embodiments, the payload region of the viral particle comprises one or more nucleic acid sequences, fragments or variants encoding MAB979, fragments or variants thereof for treating a disease and/or disorder or preventing a disease and/or disorder. As a non-limiting example, the disease and/or disorder is EV71.

In some embodiments, the payload region of the viral particle comprises one or more nucleic acid sequences encoding one or more of the payload antibody polypeptides listed in Table 40 of U.S. provisional patent application 62/844,433 against Rubella Virus (RUBV1-RUBV4; SEQ ID NO: 26250-26253), the contents of which are herein incorporated by reference in their entirety.

In some embodiments, the payload region of the viral particle comprises one or more nucleic acid sequences encoding one or more of the payload antibody polypeptides listed in Table 41 of U.S. provisional patent application 62/844,433 against Human Papilloma Virus (HPV1-HPV2; SEQ ID NO: 6896-6897), the contents of which are herein incorporated by reference in their entirety.

In some embodiments, the payload region of the viral particle comprises one or more nucleic acid sequences, fragments or variants thereof or encodes one or more polypeptides, fragments or variants thereof described in US Publication No. US20130337438, the contents of which are herein incorporated by reference in their entirety, against HPV.

In some embodiments, the payload region of the viral particle comprises one or more nucleic acid sequences encoding one or more of the broadly neutralizing payload antibody polypeptides listed in Table 42 of U.S. provisional patent application 62/844,433 against viruses (VIR1-VIR14; SEQ ID NO: 26256-26269), the contents of which are herein incorporated by reference in their entirety.

In some embodiments, the payload region of the viral particle comprises one or more nucleic acid sequences encoding one or more of the payload antibody polypeptides listed in Table 43 of U.S. provisional patent application 62/844,433 against Pseudomonas Aeruginosa (PSEU1-PSEU285; SEQ ID NO: 26270-26554), the contents of which are herein incorporated by reference in their entirety.

In some embodiments, the payload region of the viral particle comprises one or more nucleic acid sequences encoding one or more of the payload antibody polypeptides listed in Table 44 of U.S. provisional patent application 62/844,433 against Streptococcus bacteria (STRP1-STRP40; SEQ ID NO: 26555-26594), the contents of which are herein incorporated by reference in their entirety.

In some embodiments, the payload region of the viral particle comprises one or more nucleic acid sequences, fragment or variants thereof or encodes one or more polypeptides, fragments or variants thereof described in US Pub No. US20040198960 and US20130195876, the contents of each of which are herein incorporated by reference in their entirety, against Streptococcus Pneunioniae infection.

In some embodiments, the payload region of the viral particle comprises one or more nucleic acid sequences, fragments or variants encoding Afelimomab, fragments or variants thereof for treating a disease and/or disorder or preventing a disease and/or disorder. As a non-limiting example, the disease and/or disorder is sepsis.

In some embodiments, the payload region of the viral particle comprises one or more nucleic acid sequences, fragments or variants encoding Nebacumab, fragments or variants thereof for treating a disease and/or disorder or preventing a disease and/or disorder. As a non-limiting example, the disease and/or disorder is sepsis.

In some embodiments, the payload region of the viral particle comprises one or more nucleic acid sequences encoding one or more of the payload antibody polypeptides listed in Table 45 of U.S. provisional patent application 62/844,433 against Staphylococcal bacteria and related bacteria (STPH1-STPH249; SEQ ID NO: 26595-26843), the contents of which are herein incorporated by reference in their entirety.

In some embodiments, the payload region of the viral particle comprises one or more nucleic acid sequences, fragments or variants thereof or encodes one or more polypeptides, fragments or variants thereof described in International Publication No. WO2000071585, WO2013162751, WO2015089502, WO2015088346 (e.g., SEQ ID NO: 17), US Pub No. US20030224000, US20080014202, US20140037650, US20140170134, U.S. Pat. No. 8,460,666, the contents of each of which are herein incorporated by reference in their entirety, against Staphylococcus infection.

In some embodiments, the payload region of the viral particle comprises one or more nucleic acid sequences encoding one or more of the payload antibody polypeptides listed in Table 46 of U.S. provisional patent application 62/844,433 against Clostridium Tetani (CTET1-CTET57; SEQ ID NO: 26844-26900), the contents of which are herein incorporated by reference in their entirety.

In some embodiments, the payload region of the viral particle comprises one or more nucleic acid sequences encoding one or more of the payload antibody polypeptides listed in Table 47 of U.S. provisional patent application 62/844,433 against Bordetella Pertussis and/or Bordetella Parapertussis (BORT1-BORT25; SEQ ID NO: 26901-26925), the contents of which are herein incorporated by reference in their entirety.

In some embodiments, the payload region of the viral particle comprises one or more nucleic acid sequences encoding one or more of the payload antibody polypeptides listed in Table 48 of U.S. provisional patent application 62/844,433 against Mycobacteria (MYCO1-MYCO16; SEQ ID NO: 26926-26941), the contents of which are herein incorporated by reference in their entirety.

In some embodiments, the payload region of the viral particle comprises one or more nucleic acid sequences encoding one or more of the payload antibody polypeptides listed in Table 49 of U.S. provisional patent application 62/844,433 against Francisella Tularensis (FRAN1-FRAN16; SEQ ID NO: 26942-26957), the contents of which are herein incorporated by reference in their entirety.

In some embodiments, the payload region of the viral particle comprises one or more nucleic acid sequences encoding one or more of the payload antibody polypeptides listed in Table 50 of U.S. provisional patent application 62/844,433 against Bacteria (BACI1-BACI24; SEQ ID NO: 26958-26981), the contents of which are herein incorporated by reference in their entirety.

In some embodiments, the payload region of the viral particle comprises one or more nucleic acid sequences, fragments or variants encoding Doxorubicin, fragments or variants thereof for treating a disease and/or disorder or preventing a disease and/or disorder. As a non-limiting example, the disease and/or disorder is bacterial infection.

In some embodiments, the payload region of the viral particle comprises one or more nucleic acid sequences encoding one or more of the payload antibody polypeptides listed in Table 51 of U.S. provisional patent application 62/844,433 against Toxoplasma gondii (TOXO1-TOXO2; SEQ ID NO: 26982-26983), the contents of which are herein incorporated by reference in their entirety.

In some embodiments, the payload region of the viral particle comprises one or more nucleic acid sequences encoding one or more of the payload antibody polypeptides listed in Table 52 of U.S. provisional patent application 62/844,433 against Candida Yeast (CAND1; SEQ ID NO: 26984), the contents of which are herein incorporated by reference in their entirety.

In some embodiments, the payload region of the viral particle comprises one or more nucleic acid sequences encoding one or more of the payload antibody polypeptides listed in Table 53 of U.S. provisional patent application 62/844,433 (HIV1-HIV1601; SEQ ID NO: 26985-28585), the contents of which are herein incorporated by reference in their entirety.

In some embodiments, the payload region of the viral particle comprises one or more nucleic acid sequences, fragments or variants thereof or encodes one or more polypeptides, fragments or variants thereof described in European Patent Publication No. EP327000, EP478689, EP554401, EP581353 and EP711439, US Publication No. US20110104163, US20110212106, US20130215726 and US20130251726, U.S. Pat. Nos. 5,266,479, 5,804,440, 6,657,050, 8,637,036, and 9,090,675, and International Publication No. WO2012154312, WO2013163427, WO2014043386, WO2015048462, WO2015048610, WO2015048770 the contents of each of which are herein incorporated by reference in their entirety, against HIV.

### Payload Antibodies: Non-Infectious Disease

### Antibodies for the Treatment of Cancer and Immunoinflammatory Diseases

In some embodiments, the payload region of the AAV particle comprises one or more nucleic acid sequences encoding cancer and immunoinflammatory diseases-associated antibodies, variants or fragments thereof.

In some embodiments, the payload region of the AAV particle comprises one or more nucleic acid sequences encoding one or more of the payload antibody polypeptides listed in Table 4, or variants or fragments thereof.

In some embodiments, the payload region of the AAV particle comprises one or more nucleic acid sequences listed in Table 4, or variants or fragments thereof.

In some embodiments, the payload region of the AAV particle comprises a nucleic acid sequence encoding a payload antibody with at least 50% identity to one or more payload antibody polypeptides listed in Table 4. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 4, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 4, or variants or fragments thereof.

In some embodiments, the variable region sequence(s) of the encoded antibody polypeptide may have 50%, 51%, 52%, 53%, 54%, 55%, 56%, 57%, 58%, 59%,60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%,84%,85%, 86%, 87%, 88%, 89%, 90%, 91%, 92%, 93%, 94%, 95%, 96%, 97%, 98%, 99%, or 100% identity to one or more of the payload antibody polypeptides listed in Table 4, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 4, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%%,%, 87%,88%, 89%, 90%,91%, 92%, 93%,94%,95%, 96%,97%,98%, 99%, or 100% identity to one or more of the payload light chain antibody polypeptides listed in Table 4, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%,63%,64%, 65%, 66%,67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 4, or variants or fragments thereof.

In some embodiments, the payload antibody has 90% identity to one or more of the antibody polypeptides listed in Table 4, or variants or fragments thereof.

In some embodiments, the payload antibody has 91% identity to one or more of the antibody polypeptides listed in Table 4, or variants or fragments thereof.

In some embodiments, the payload antibody has 92% identity to one or more of the antibody polypeptides listed in Table 4, or variants or fragments thereof.

In some embodiments, the payload antibody has 93% identity to one or more of the antibody polypeptides listed in Table 4, or variants or fragments thereof.

In some embodiments, the payload antibody has 94% identity to one or more of the antibody polypeptides listed in Table 4, or variants or fragments thereof.

In some embodiments, the payload antibody has 95% identity to one or more of the antibody polypeptides listed in Table 4, or variants or fragments thereof.

In some embodiments, the payload antibody has 96% identity to one or more of the antibody polypeptides listed in Table 4, or variants or fragments thereof.

In some embodiments, the payload antibody has 97% identity to one or more of the antibody polypeptides listed in Table 4, or variants or fragments thereof.

In some embodiments, the payload antibody has 98% identity to one or more of the antibody polypeptides listed in Table 4, or variants or fragments thereof.

In some embodiments, the payload antibody has 99% identity to one or more of the antibody polypeptides listed in Table 4, or variants or fragments thereof.

In some embodiments, the payload antibody has 100% identity to one or more of the antibody polypeptides listed in Table 4, or variants or fragments thereof.

In some embodiments, the payload antibody may be variants of any of the antibody polypeptides listed in Table 4, that exclude one or more amino acids designated as “X” or “x” in the described polypeptide sequence, wherein X may represent any amino acid. In some embodiments, the payload nucleic acid sequence may be variants of any of the nucleic acid sequences listed in Table 4, that exclude one or more nucleic acids designated as “n” or “N” in the described nucleic acid sequence, wherein n may represent any nucleic acid.

TABLE 4
Cancer and Immunoinflammatory Antibodies

| | | Type | | SEQ |
|---|---|---|---|---|
| Ab ID | Description | (PRT/DNA) | Reference | ID NO |
| CII1 | CDR | PRT | WO2014028776; SEQ ID NO: 58 | 2168 |
| CII2 | CDR | PRT | WO2014028776; SEQ ID NO: 57 | 2169 |
| CII3 | CDR | PRT | WO2014028776; SEQ ID NO: 55 | 2170 |
| CII4 | CDR | PRT | WO2014028776; SEQ ID NO: 59 | 2171 |
| CII5 | CDR | PRT | WO2014028776; SEQ ID NO: 56 | 2172 |
| CII6 | CDR | PRT | WO2014028776; SEQ ID NO: 60 | 2173 |
| CII7 | CDR | PRT | US20180134806; SEQ ID NO: 13 | 2174 |
| CII8 | CDR | PRT | US20180134806; SEQ ID NO: 62 | 2175 |
| CII9 | CDR | PRT | US20180134806; SEQ ID NO: 63 | 2176 |
| CII10 | CDR | PRT | US20180134806; SEQ ID NO: 59 | 2177 |
| CII11 | CDR | PRT | US20180134806; SEQ ID NO: 61 | 2178 |
| CII12 | CDR | PRT | US20180134806; SEQ ID NO: 60 | 2179 |
| CII13 | CDR | PRT | US20180134806; SEQ ID NO: 9 | 2180 |
| CII14 | CDR | PRT | US20180134806; SEQ ID NO: 39 | 2181 |
| CII15 | CDR | PRT | US20180134806; SEQ ID NO: 43 | 2182 |
| CII16 | CDR | PRT | US20180134806; SEQ ID NO: 40 | 2183 |
| CII17 | CDR | PRT | US20180134806; SEQ ID NO: 42 | 2184 |
| CII18 | CDR | PRT | US20180134806; SEQ ID NO: 41 | 2185 |
| CII19 | CDR | PRT | US20180134806; SEQ ID NO: 10 | 2186 |
| CII20 | CDR | PRT | US20180134806; SEQ ID NO: 53 | 2187 |
| CII21 | CDR | PRT | US20180134806; SEQ ID NO: 56 | 2188 |
| CII22 | CDR | PRT | US20180134806; SEQ ID NO: 58 | 2189 |
| CII23 | CDR | PRT | US20180134806; SEQ ID NO: 57 | 2190 |
| CII24 | CDR | PRT | US20180134806; SEQ ID NO: 54 | 2191 |
| CII25 | CDR | PRT | US20180134806; SEQ ID NO: 55 | 2192 |
| CII26 | CDR | PRT | US20180134806; SEQ ID NO: 52 | 2193 |
| CII27 | CDR | PRT | US20180134806; SEQ ID NO: 14 | 2194 |
| CII28 | CDR | PRT | US20180134806; SEQ ID NO: 31 | 2195 |
| CII29 | CDR | PRT | US20180134806; SEQ ID NO: 32 | 2196 |
| CII30 | CDR | PRT | US20180134806; SEQ ID NO: 35 | 2197 |
| CII31 | CDR | PRT | US20180134806; SEQ ID NO: 34 | 2198 |
| CII32 | CDR | PRT | US20180134806; SEQ ID NO: 38 | 2199 |
| CII33 | CDR | PRT | US20180134806; SEQ ID NO: 37 | 2200 |
| CII34 | CDR | PRT | US20180134806; SEQ ID NO: 36 | 2201 |
| CII35 | CDR | PRT | US20180134806; SEQ ID NO: 33 | 2202 |
| CII36 | CDR | PRT | US20180134806; SEQ ID NO: 29 | 2203 |
| CII37 | CDR | PRT | US20180134806; SEQ ID NO: 30 | 2204 |
| CII38 | CDR | PRT | US20180134806; SEQ ID NO: 7 | 2205 |
| CII39 | CDR | PRT | US20180134806; SEQ ID NO: 8 | 2206 |
| CII40 | CDR | PRT | US20180134806; SEQ ID NO: 6 | 2207 |
| CII41 | CDR | PRT | US20180134806; SEQ ID NO: 47 | 2208 |
| CII42 | CDR | PRT | US20180134806; SEQ ID NO: 46 | 2209 |
| CII43 | CDR | PRT | US20180134806; SEQ ID NO: 44 | 2210 |
| CII44 | CDR | PRT | US20180134806; SEQ ID NO: 51 | 2211 |
| CII45 | CDR | PRT | US20180134806; SEQ ID NO: 45 | 2212 |
| CII46 | CDR | PRT | US20180134806; SEQ ID NO: 50 | 2213 |
| CII47 | CDR | PRT | US20180134806; SEQ ID NO: 11 | 2214 |
| CII48 | CDR | PRT | US20180134806; SEQ ID NO: 49 | 2215 |
| CII49 | CDR | PRT | US20180134806; SEQ ID NO: 48 | 2216 |
| CII50 | CDR | PRT | US20180134806; SEQ ID NO: 12 | 2217 |
| CII51 | CDR | PRT | US20190085076; SEQ ID NO: 37 | 2218 |
| CII52 | CDR | PRT | W02016201389; SEQ ID NO: 37 | 2219 |
| CII53 | CDR | PRT | US20190085076; SEQ ID NO: 28 | 2220 |
| CII54 | CDR | PRT | W02016201389; SEQ ID NO: 28 | 2221 |
| CII55 | CDR | PRT | W02016201389; SEQ ID NO: 158 | 2222 |
| CII56 | CDR | PRT | US20190085076; SEQ ID NO: 137 | 2223 |
| CII57 | CDR | PRT | W02016201389; SEQ ID NO: 137 | 2224 |
| CII58 | CDR | PRT | W02016201389; SEQ ID NO: 165 | 2225 |
| CII59 | CDR | PRT | US20190085076; SEQ ID NO: 213 | 2226 |
| CII60 | CDR | PRT | W02016201389; SEQ ID NO: 213 | 2227 |
| CII61 | CDR | PRT | W02016201389; SEQ ID NO: 168 | 2228 |
| CII62 | CDR | PRT | W02016201389; SEQ ID NO: 216 | 2229 |
| CII63 | CDR | PRT | W02016201389; SEQ ID NO: 162 | 2230 |
| CII64 | CDR | PRT | US20190085076; SEQ ID NO: 173 | 2231 |
| CII65 | CDR | PRT | W02016201389; SEQ ID NO: 173 | 2232 |
| CII66 | CDR | PRT | W02016201389; SEQ ID NO: 217 | 2233 |
| CII67 | CDR | PRT | W02016201389; SEQ ID NO: 218 | 2234 |
| CII68 | CDR | PRT | W02016201389; SEQ ID NO: 229 | 2235 |
| CII69 | CDR | PRT | W02016201389; SEQ ID NO: 164 | 2236 |
| CII70 | CDR | PRT | W02016201389; SEQ ID NO: 201 | 2237 |
| CII71 | CDR | PRT | W02016201389; SEQ ID NO: 161 | 2238 |
| CII72 | CDR | PRT | US20190085076; SEQ ID NO: 211 | 2239 |
| CII73 | CDR | PRT | W02016201389; SEQ ID NO: 211 | 2240 |
| CII74 | CDR | PRT | US20190085076; SEQ ID NO: 190 | 2241 |
| CII75 | CDR | PRT | W02016201389; SEQ ID NO: 190 | 2242 |
| CII76 | CDR | PRT | W02016201389; SEQ ID NO: 176 | 2243 |
| CII77 | CDR | PRT | W02016201389; SEQ ID NO: 167 | 2244 |
| CII78 | CDR | PRT | US20190085076; SEQ ID NO: 191 | 2245 |
| CII79 | CDR | PRT | W02016201389; SEQ ID NO: 191 | 2246 |
| CII80 | CDR | PRT | US20190085076; SEQ ID NO: 179 | 2247 |
| CII81 | CDR | PRT | W02016201389; SEQ ID NO: 179 | 2248 |
| CII82 | CDR | PRT | US20190085076; SEQ ID NO: 170 | 2249 |
| CII83 | CDR | PRT | W02016201389; SEQ ID NO: 170 | 2250 |
| CII84 | CDR | PRT | W02016201389; SEQ ID NO: 220 | 2251 |
| CII85 | CDR | PRT | W02016201389; SEQ ID NO: 178 | 2252 |
| CII86 | CDR | PRT | US20190085076; SEQ ID NO: 189 | 2253 |
| CII87 | CDR | PRT | W02016201389; SEQ ID NO: 189 | 2254 |
| CII88 | CDR | PRT | W02016201389; SEQ ID NO: 221 | 2255 |
| CII89 | CDR | PRT | W02016201389; SEQ ID NO: 206 | 2256 |
| CII90 | CDR | PRT | W02016201389; SEQ ID NO: 210 | 2257 |
| CII91 | CDR | PRT | W02016201389; SEQ ID NO: 172 | 2258 |
| CII92 | CDR | PRT | US20190085076; SEQ ID NO: 222 | 2259 |
| CII93 | CDR | PRT | W02016201389; SEQ ID NO: 222 | 2260 |
| CII94 | CDR | PRT | W02016201389; SEQ ID NO: 199 | 2261 |
| CII95 | CDR | PRT | US20190085076; SEQ ID NO: 175 | 2262 |
| CII96 | CDR | PRT | W02016201389; SEQ ID NO: 175 | 2263 |
| CII97 | CDR | PRT | US20190085076; SEQ ID NO: 163 | 2264 |
| CII98 | CDR | PRT | W02016201389; SEQ ID NO: 163 | 2265 |
| CII99 | CDR | PRT | US20190085076; SEQ ID NO: 226 | 2266 |
| CII100 | CDR | PRT | W02016201389; SEQ ID NO: 226 | 2267 |
| CII101 | CDR | PRT | W02016201389; SEQ ID NO: 166 | 2268 |
| CII102 | CDR | PRT | US20190085076; SEQ ID NO: 203 | 2269 |
| CII103 | CDR | PRT | W02016201389; SEQ ID NO: 203 | 2270 |
| CII104 | CDR | PRT | US20190085076; SEQ ID NO: 225 | 2271 |
| CII105 | CDR | PRT | W02016201389; SEQ ID NO: 225 | 2272 |
| CII106 | CDR | PRT | US20199085076; SEQ ID NO: 193 | 2273 |
| CII107 | CDR | PRT | W02016201389; SEQ ID NO: 193 | 2274 |
| CII108 | CDR | PRT | US20190085076; SEQ ID NO: 185 | 2275 |
| CII109 | CDR | PRT | W02016201389; SEQ ID NO: 185 | 2276 |
| CII110 | CDR | PRT | US20190085076; SEQ ID NO: 205 | 2277 |
| CII111 | CDR | PRT | W02016201389; SEQ ID NO: 205 | 2278 |
| CII112 | CDR | PRT | US20199085076; SEQ ID NO: 182 | 2279 |
| CII113 | CDR | PRT | W02016201389; SEQ ID NO: 182 | 2280 |
| CII114 | CDR | PRT | US20190085076; SEQ ID NO: 196 | 2281 |
| CII115 | CDR | PRT | W02016201389; SEQ ID NO: 196 | 2282 |
| CII116 | CDR | PRT | US20190085076; SEQ ID NO: 195 | 2283 |
| CII117 | CDR | PRT | W02016201389; SEQ ID NO: 195 | 2284 |
| CII118 | CDR | PRT | US20190085076; SEQ ID NO: 224 | 2285 |
| CII119 | CDR | PRT | W02016201389; SEQ ID NO: 224 | 2286 |
| CII120 | CDR | PRT | US29190085076; SEQ ID NO: 188 | 2287 |
| CII121 | CDR | PRT | W02016201389; SEQ ID NO: 188 | 2288 |
| CII122 | CDR | PRT | US20190085076; SEQ ID NO: 184 | 2289 |
| CII123 | CDR | PRT | W02016201389; SEQ ID NO: 184 | 2290 |
| CII124 | CDR | PRT | US20190085076; SEQ ID NO: 174 | 2291 |
| CII125 | CDR | PRT | W02016201389; SEQ ID NO: 174 | 2292 |
| CII126 | CDR | PRT | US20190085076; SEQ ID NO: 194 | 2293 |
| CII127 | CDR | PRT | W02016201389; SEQ ID NO: 194 | 2294 |
| CII128 | CDR | PRT | US20190085076; SEQ ID NO: 198 | 2295 |
| CII129 | CDR | PRT | W02016201389; SEQ ID NO: 198 | 2296 |
| CII130 | CDR | PRT | US20190085076; SEQ ID NO: 192 | 2297 |
| CII131 | CDR | PRT | W02016201389; SEQ ID NO: 192 | 2298 |
| CII132 | CDR | PRT | US20190085076; SEQ ID NO: 197 | 2299 |
| CII133 | CDR | PRT | W02016201389; SEQ ID NO: 197 | 2300 |
| CII134 | CDR | PRT | US20190085076; SEQ ID NO: 169 | 2301 |
| CII135 | CDR | PRT | W02016201389; SEQ ID NO: 169 | 2302 |
| CII136 | CDR | PRT | US20190085076; SEQ ID NO: 181 | 2303 |
| CII137 | CDR | PRT | W02016201389; SEQ ID NO: 181 | 2304 |
| CII138 | CDR | PRT | W02016201389; SEQ ID NO: 180 | 2305 |
| CII139 | CDR | PRT | W02016201389; SEQ ID NO: 208 | 2306 |
| CII140 | CDR | PRT | US20190085076; SEQ ID NO: 209 | 2307 |
| CII141 | CDR | PRT | W02016201389; SEQ ID NO: 209 | 2308 |
| CII142 | CDR | PRT | W02016201389; SEQ ID NO: 202 | 2309 |
| CII143 | CDR | PRT | W02016201389; SEQ ID NO: 228 | 2310 |
| CII144 | CDR | PRT | US20190085076; SEQ ID NO: 204 | 2311 |
| CII145 | CDR | PRT | W02016201389; SEQ ID NO: 204 | 2312 |
| CII146 | CDR | PRT | US20190085076; SEQ ID NO: 187 | 2313 |
| CII147 | CDR | PRT | W02016201389; SEQ ID NO: 187 | 2314 |
| CII148 | CDR | PRT | US20190085076; SEQ ID NO: 171 | 2315 |
| CII149 | CDR | PRT | W02016201389; SEQ ID NO: 171 | 2316 |
| CII150 | CDR | PRT | W02016201389; SEQ ID NO: 200 | 2317 |
| CII151 | CDR | PRT | W02016201389; SEQ ID NO: 212 | 2318 |
| CII152 | CDR | PRT | US20190085076; SEQ ID NO: 186 | 2319 |
| CII153 | CDR | PRT | W02016201389; SEQ ID NO: 186 | 2320 |
| CII154 | CDR | PRT | W02016201389; SEQ ID NO: 207 | 2321 |
| CII155 | CDR | PRT | US20190085076; SEQ ID NO: 214 | 2322 |
| CII156 | CDR | PRT | W02016201389; SEQ ID NO: 214 | 2323 |
| CII157 | CDR | PRT | W02016201389; SEQ ID NO: 227 | 2324 |
| CII158 | CDR | PRT | W02016201389; SEQ ID NO: 434 | 2325 |
| CII159 | CDR | PRT | W02016201389; SEQ ID NO: 215 | 2326 |
| CII160 | CDR | PRT | US20190085076; SEQ ID NO: 223 | 2327 |
| CII161 | CDR | PRT | W02016201389; SEQ ID NO: 223 | 2328 |
| CII162 | CDR | PRT | US20190085076; SEQ ID NO: 183 | 2329 |
| CII163 | CDR | PRT | W02016201389; SEQ ID NO: 183 | 2330 |
| CII164 | CDR | PRT | W02016201388; SEQ ID NO: 27 | 2331 |
| CII165 | CDR | PRT | W02016201389; SEQ ID NO: 230 | 2332 |
| CII166 | CDR | PRT | US20190085076; SEQ ID NO: 177 | 2333 |
| CII167 | CDR | PRT | W02016201389; SEQ ID NO: 177 | 2334 |
| CII168 | CDR | PRT | W02016201389; SEQ ID NO: 219 | 2335 |
| CII169 | CDR | PRT | US20190085076; SEQ ID NO: 35 | 2336 |
| CII170 | CDR | PRT | W02016201389; SEQ ID NO: 35 | 2337 |
| CII171 | CDR | PRT | US20190085076; SEQ ID NO: 31 | 2338 |
| CII172 | CDR | PRT | W02016201389; SEQ ID NO: 31 | 2339 |
| CII173 | CDR | PRT | US20190085076; SEQ ID NO: 33 | 2340 |
| CII174 | CDR | PRT | W02016201389; SEQ ID NO: 33 | 2341 |
| CII175 | CDR | PRT | US20190085076; SEQ ID NO: 24 | 2342 |
| CII176 | CDR | PRT | W02016201389; SEQ ID NO: 24 | 2343 |
| CII177 | CDR | PRT | W02016201389; SEQ ID NO: 29 | 2344 |
| CII178 | CDR | PRT | WO2016201388; SEQ ID NO: 188 | 2345 |
| CII179 | CDR | PRT | US20190085076; SEQ ID NO: 32 | 2346 |
| CII180 | CDR | PRT | W02016201389; SEQ ID NO: 32 | 2347 |
| CII181 | CDR | PRT | WO2019028283; SEQ ID NO: 190 | 2348 |
| CII182 | CDR | PRT | WO2019028283; SEQ ID NO: 148 | 2349 |
| CII183 | CDR | PRT | WO2016201388; SEQ ID NO: 191 | 2350 |
| CII184 | CDR | PRT | WO2019028283; SEQ ID NO: 116 | 2351 |
| CII185 | CDR | PRT | WO2019028283; SEQ ID NO: 117 | 2352 |
| CII186 | CDR | PRT | WO2019028283; SEQ ID NO: 115 | 2353 |
| CII187 | CDR | PRT | WO2019028283; SEQ ID NO: 194 | 2354 |
| CII188 | CDR | PRT | WO2019028283; SEQ ID NO: 118 | 2355 |
| CII189 | CDR | PRT | WO2019028283; SEQ ID NO: 196 | 2356 |
| CII190 | CDR | PRT | WO2019028283; SEQ ID NO: 119 | 2357 |
| CII191 | CDR | PRT | WO2019028283; SEQ ID NO: 195 | 2358 |
| CII192 | CDR | PRT | WO2019028283; SEQ ID NO: 120 | 2359 |
| CII193 | CDR | PRT | WO2019028283; SEQ ID NO: 153 | 2360 |
| CII194 | CDR | PRT | US20190085076; SEQ ID NO: 121 | 2361 |
| CII195 | CDR | PRT | W02016201389; SEQ ID NO: 121 | 2362 |
| CII196 | CDR | PRT | WO2016201388; SEQ ID NO: 21 | 2363 |
| CII197 | CDR | PRT | W02016201389; SEQ ID NO: 119 | 2364 |
| CII198 | CDR | PRT | W02016201389; SEQ ID NO: 116 | 2365 |
| CII199 | CDR | PRT | US20190085076; SEQ ID NO: 124 | 2366 |
| CII200 | CDR | PRT | W02016201389; SEQ ID NO: 124 | 2367 |
| CII201 | CDR | PRT | US20190085076; SEQ ID NO: 130 | 2368 |
| CII202 | CDR | PRT | W02016201389; SEQ ID NO: 130 | 2369 |
| CII203 | CDR | PRT | US20190085076; SEQ ID NO: 122 | 2370 |
| CII204 | CDR | PRT | W02016201389; SEQ ID NO: 122 | 2371 |
| CII205 | CDR | PRT | W02016201389; SEQ ID NO: 134 | 2372 |
| CII206 | CDR | PRT | W02016201389; SEQ ID NO: 133 | 2373 |
| CII207 | CDR | PRT | US20190085076; SEQ ID NO: 34 | 2374 |
| CII208 | CDR | PRT | W02016201389; SEQ ID NO: 34 | 2375 |
| CII209 | CDR | PRT | US20190085076; SEQ ID NO: 27 | 2376 |
| CII210 | CDR | PRT | W02016201389; SEQ ID NO: 27 | 2377 |
| CII211 | CDR | PRT | US20190085076; SEQ ID NO: 26 | 2378 |
| CII212 | CDR | PRT | W02016201389; SEQ ID NO: 26 | 2379 |
| CII213 | CDR | PRT | WO2019028283; SEQ ID NO: 106 | 2380 |
| CII214 | CDR | PRT | WO2019028283; SEQ ID NO: 107 | 2381 |
| CII215 | CDR | PRT | US20190085076; SEQ ID NO: 139 | 2382 |
| CII216 | CDR | PRT | W02016201389; SEQ ID NO: 139 | 2383 |
| CII217 | CDR | PRT | US20190085076; SEQ ID NO: 125 | 2384 |
| CII218 | CDR | PRT | W02016201389; SEQ ID NO: 125 | 2385 |
| CII219 | CDR | PRT | US20190085076; SEQ ID NO: 129 | 2386 |
| CII220 | CDR | PRT | W02016201389; SEQ ID NO: 129 | 2387 |
| CII221 | CDR | PRT | US20190085076; SEQ ID NO: 126 | 2388 |
| CII222 | CDR | PRT | W02016201389; SEQ ID NO: 126 | 2389 |
| CII223 | CDR | PRT | US20190085076; SEQ ID NO: 120 | 2390 |
| CII224 | CDR | PRT | W02016201389; SEQ ID NO: 120 | 2391 |
| CII225 | CDR | PRT | US20190085076; SEQ ID NO: 117 | 2392 |
| CII226 | CDR | PRT | W02016201389; SEQ ID NO: 117 | 2393 |
| CII227 | CDR | PRT | W02016201389; SEQ ID NO: 132 | 2394 |
| CII228 | CDR | PRT | W02016201389; SEQ ID NO: 131 | 2395 |
| CII229 | CDR | PRT | WO2016201388; SEQ ID NO: 230 | 2396 |
| CII230 | CDR | PRT | WO2016201388; SEQ ID NO: 189 | 2397 |
| CII231 | CDR | PRT | WO2019028283; SEQ ID NO: 110 | 2398 |
| CII232 | CDR | PRT | WO2019028283; SEQ ID NO: 152 | 2399 |
| CII233 | CDR | PRT | WO2019028283; SEQ ID NO: 186 | 2400 |
| CII234 | CDR | PRT | WO2019028283; SEQ ID NO: 111 | 2401 |
| CII235 | CDR | PRT | WO2019028283; SEQ ID NO: 112 | 2402 |
| CII236 | CDR | PRT | WO2019028283; SEQ ID NO: 113 | 2403 |
| CII237 | CDR | PRT | WO2019028283; SEQ ID NO: 105 | 2404 |
| CII238 | CDR | PRT | WO2019028283; SEQ ID NO: 109 | 2405 |
| CII239 | CDR | PRT | WO2019028283; SEQ ID NO: 108 | 2406 |
| CII240 | CDR | PRT | WO2019028283; SEQ ID NO: 114 | 2407 |
| CII241 | CDR | PRT | WO2016201388; SEQ ID NO: 24 | 2408 |
| CII242 | CDR | PRT | WO2016201388; SEQ ID NO: 23 | 2409 |
| CII243 | CDR | PRT | WO2016201388; SEQ ID NO: 186 | 2410 |
| CII244 | CDR | PRT | WO2016201388; SEQ ID NO: 77 | 2411 |
| CII245 | CDR | PRT | WO2016201388; SEQ ID NO: 22 | 2412 |
| CII246 | CDR | PRT | W02016201389; SEQ ID NO: 159 | 2413 |
| CII247 | CDR | PRT | WO2016201388; SEQ ID NO: 231 | 2414 |
| CII248 | CDR | PRT | WO2016201388; SEQ ID NO: 9 | 2415 |
| CII249 | CDR | PRT | WO2019028283; SEQ ID NO: 131 | 2416 |
| CII250 | CDR | PRT | W02016201389; SEQ ID NO: 10 | 2417 |
| CII251 | CDR | PRT | US20190085076; SEQ ID NO: 38 | 2418 |
| CII252 | CDR | PRT | W02016201389; SEQ ID NO: 38 | 2419 |
| CII253 | CDR | PRT | WO2016201388; SEQ ID NO: 14 | 2420 |
| CII254 | CDR | PRT | WO2016201388; SEQ ID NO: 71 | 2421 |
| CII255 | CDR | PRT | US20190085076; SEQ ID NO: 36 | 2422 |
| CII256 | CDR | PRT | W02016201389; SEQ ID NO: 36 | 2423 |
| CII257 | CDR | PRT | WO2019028283; SEQ ID NO: 192 | 2424 |
| CII258 | CDR | PRT | US20190085076; SEQ ID NO: 88 | 2425 |
| CII259 | CDR | PRT | W02016201389; SEQ ID NO: 88 | 2426 |
| CII260 | CDR | PRT | WO2016201388; SEQ ID NO: 15 | 2427 |
| CII261 | CDR | PRT | WO2016201388; SEQ ID NO: 185 | 2428 |
| CII262 | CDR | PRT | WO2016201388; SEQ ID NO: 70 | 2429 |
| CII263 | CDR | PRT | WO2016201388; SEQ ID NO: 229 | 2430 |
| CII264 | CDR | PRT | W02016201389; SEQ ID NO: 433 | 2431 |
| CII265 | CDR | PRT | US20190085076; SEQ ID NO: 80 | 2432 |
| CII266 | CDR | PRT | W02016201389; SEQ ID NO: 80 | 2433 |
| CII267 | CDR | PRT | US20190085076; SEQ ID NO: 67 | 2434 |
| CII268 | CDR | PRT | W02016201389; SEQ ID NO: 67 | 2435 |
| CII269 | CDR | PRT | US20190085076; SEQ ID NO: 151 | 2436 |
| CII270 | CDR | PRT | W02016201389; SEQ ID NO: 151 | 2437 |
| CII271 | CDR | PRT | US20190085076; SEQ ID NO: 142 | 2438 |
| CII272 | CDR | PRT | W02016201389; SEQ ID NO: 142 | 2439 |
| CII273 | CDR | PRT | US20190085076; SEQ ID NO: 145 | 2440 |
| CII274 | CDR | PRT | W02016201389; SEQ ID NO: 145 | 2441 |
| CII275 | CDR | PRT | US20190085076; SEQ ID NO: 146 | 2442 |
| CII276 | CDR | PRT | W02016201389; SEQ ID NO: 146 | 2443 |
| CII277 | CDR | PRT | WO2016201388; SEQ ID NO: 73 | 2444 |
| CII278 | CDR | PRT | W02016201389; SEQ ID NO: 22 | 2445 |
| CII279 | CDR | PRT | US20190085076; SEQ ID NO: 16 | 2446 |
| CII280 | CDR | PRT | W02016201389; SEQ ID NO: 16 | 2447 |
| CII281 | CDR | PRT | US20190085076; SEQ ID NO: 19 | 2448 |
| CII282 | CDR | PRT | W02016201389; SEQ ID NO: 19 | 2449 |
| CII283 | CDR | PRT | W02016201389; SEQ ID NO: 83 | 2450 |
| CII284 | CDR | PRT | WO2019028283; SEQ ID NO: 151 | 2451 |
| CII285 | CDR | PRT | WO2016201388; SEQ ID NO: 17 | 2452 |
| CII286 | CDR | PRT | WO2016201388; SEQ ID NO: 74 | 2453 |
| CII287 | CDR | PRT | WO2019028283; SEQ ID NO: 150 | 2454 |
| CII288 | CDR | PRT | WO2019028283; SEQ ID NO: 147 | 2455 |
| CII289 | CDR | PRT | WO2016201388; SEQ ID NO: 72 | 2456 |
| CII290 | CDR | PRT | WO2019028283; SEQ ID NO: 146 | 2457 |
| CII291 | CDR | PRT | WO2019028283; SEQ ID NO: 149 | 2458 |
| CII292 | CDR | PRT | W02016201389; SEQ ID NO: 84 | 2459 |
| CII293 | CDR | PRT | W02016201389; SEQ ID NO: 52 | 2460 |
| CII294 | CDR | PRT | W02016201389; SEQ ID NO: 56 | 2461 |
| CII295 | CDR | PRT | W02016201389; SEQ ID NO: 45 | 2462 |
| CII296 | CDR | PRT | W02016201389; SEQ ID NO: 44 | 2463 |
| CII297 | CDR | PRT | US20190085076; SEQ ID NO: 99 | 2464 |
| CII298 | CDR | PRT | W02016201389; SEQ ID NO: 99 | 2465 |
| CII299 | CDR | PRT | W02016201389; SEQ ID NO: 60 | 2466 |
| CII300 | CDR | PRT | W02016201389; SEQ ID NO: 106 | 2467 |
| CII301 | CDR | PRT | US20190085076; SEQ ID NO: 54 | 2468 |
| CII302 | CDR | PRT | W02016201389; SEQ ID NO: 54 | 2469 |
| CII303 | CDR | PRT | W02016201389; SEQ ID NO: 82 | 2470 |
| CII304 | CDR | PRT | US20190085076; SEQ ID NO: 77 | 2471 |
| CII305 | CDR | PRT | W02016201389; SEQ ID NO: 77 | 2472 |
| CII306 | CDR | PRT | US20190085076; SEQ ID NO: 51 | 2473 |
| CII307 | CDR | PRT | W02016201389; SEQ ID NO: 51 | 2474 |
| CII308 | CDR | PRT | US20190085076; SEQ ID NO: 81 | 2475 |
| CII309 | CDR | PRT | W02016201389; SEQ ID NO: 81 | 2476 |
| CII310 | CDR | PRT | W02016201389; SEQ ID NO: 42 | 2477 |
| CII311 | CDR | PRT | W02016201389; SEQ ID NO: 105 | 2478 |
| CII312 | CDR | PRT | US20190085076; SEQ ID NO: 68 | 2479 |
| CII313 | CDR | PRT | W02016201389; SEQ ID NO: 68 | 2480 |
| CII314 | CDR | PRT | W02016201389; SEQ ID NO: 40 | 2481 |
| CII315 | CDR | PRT | W02016201389; SEQ ID NO: 102 | 2482 |
| CII316 | CDR | PRT | W02016201389; SEQ ID NO: 101 | 2483 |
| CII317 | CDR | PRT | W02016201389; SEQ ID NO: 103 | 2484 |
| CII318 | CDR | PRT | W02016201389; SEQ ID NO: 93 | 2485 |
| CII319 | CDR | PRT | US20190085076; SEQ ID NO: 76 | 2486 |
| CII320 | CDR | PRT | W02016201389; SEQ ID NO: 76 | 2487 |
| CII321 | CDR | PRT | US20190085076; SEQ ID NO: 78 | 2488 |
| CII322 | CDR | PRT | W02016201389; SEQ ID NO: 78 | 2489 |
| CII323 | CDR | PRT | W02016201389; SEQ ID NO: 91 | 2490 |
| CII324 | CDR | PRT | US20190085076; SEQ ID NO: 79 | 2491 |
| CII325 | CDR | PRT | W02016201389; SEQ ID NO: 79 | 2492 |
| CII326 | CDR | PRT | US20190085076; SEQ ID NO: 72 | 2493 |
| CII327 | CDR | PRT | W02016201389; SEQ ID NO: 72 | 2494 |
| CII328 | CDR | PRT | US20190085076; SEQ ID NO: 111 | 2495 |
| CII329 | CDR | PRT | W02016201389; SEQ ID NO: 111 | 2496 |
| CII330 | CDR | PRT | US20190085076; SEQ ID NO: 115 | 2497 |
| CII331 | CDR | PRT | W02016201389; SEQ ID NO: 115 | 2498 |
| CII332 | CDR | PRT | US20190085076; SEQ ID NO: 65 | 2499 |
| CII333 | CDR | PRT | W02016201389; SEQ ID NO: 65 | 2500 |
| CII334 | CDR | PRT | US20190085076; SEQ ID NO: 109 | 2501 |
| CII335 | CDR | PRT | W02016201389; SEQ ID NO: 109 | 2502 |
| CII336 | CDR | PRT | US20190085076; SEQ ID NO: 64 | 2503 |
| CII337 | CDR | PRT | W02016201389; SEQ ID NO: 64 | 2504 |
| CII338 | CDR | PRT | W02016201389; SEQ ID NO: 113 | 2505 |
| CII339 | CDR | PRT | US20190085076; SEQ ID NO: 57 | 2506 |
| CII340 | CDR | PRT | W02016201389; SEQ ID NO: 57 | 2507 |
| CII341 | CDR | PRT | US20190085076; SEQ ID NO: 100 | 2508 |
| CII342 | CDR | PRT | W02016201389; SEQ ID NO: 100 | 2509 |
| CII343 | CDR | PRT | US20190085076; SEQ ID NO: 108 | 2510 |
| CII344 | CDR | PRT | W02016201389; SEQ ID NO: 108 | 2511 |
| CII345 | CDR | PRT | W02016201389; SEQ ID NO: 104 | 2512 |
| CII346 | CDR | PRT | W02016201389; SEQ ID NO: 114 | 2513 |
| CII347 | CDR | PRT | US20190085076; SEQ ID NO: 62 | 2514 |
| CII348 | CDR | PRT | W02016201389; SEQ ID NO: 62 | 2515 |
| CII349 | CDR | PRT | US20190085076; SEQ ID NO: 70 | 2516 |
| CII350 | CDR | PRT | W02016201389; SEQ ID NO: 70 | 2517 |
| CII351 | CDR | PRT | US20190085076; SEQ ID NO: 71 | 2518 |
| CII352 | CDR | PRT | W02016201389; SEQ ID NO: 71 | 2519 |
| CII353 | CDR | PRT | US20190085076; SEQ ID NO: 94 | 2520 |
| CII354 | CDR | PRT | W02016201389; SEQ ID NO: 94 | 2521 |
| CII355 | CDR | PRT | US20190085076; SEQ ID NO: 110 | 2522 |
| CII356 | CDR | PRT | W02016201389; SEQ ID NO: 110 | 2523 |
| CII357 | CDR | PRT | US20190085076; SEQ ID NO: 47 | 2524 |
| CII358 | CDR | PRT | W02016201389; SEQ ID NO: 47 | 2525 |
| CII359 | CDR | PRT | US20190085076; SEQ ID NO: 63 | 2526 |
| CII360 | CDR | PRT | W02016201389; SEQ ID NO: 63 | 2527 |
| CII361 | CDR | PRT | US20190085076; SEQ ID NO: 50 | 2528 |
| CII362 | CDR | PRT | W02016201389; SEQ ID NO: 50 | 2529 |
| CII363 | CDR | PRT | W02016201389; SEQ ID NO: 85 | 2530 |
| CII364 | CDR | PRT | US20190085076; SEQ ID NO: 89 | 2531 |
| CII365 | CDR | PRT | W02016201389; SEQ ID NO: 89 | 2532 |
| CII366 | CDR | PRT | US20190085076; SEQ ID NO: 61 | 2533 |
| CII367 | CDR | PRT | W02016201389; SEQ ID NO: 61 | 2534 |
| CII368 | CDR | PRT | W02016201389; SEQ ID NO: 112 | 2535 |
| CII369 | CDR | PRT | US20190085076; SEQ ID NO: 97 | 2536 |
| CII370 | CDR | PRT | W02016201389; SEQ ID NO: 97 | 2537 |
| CII371 | CDR | PRT | W02016201389; SEQ ID NO: 46 | 2538 |
| CII372 | CDR | PRT | W02016201389; SEQ ID NO: 58 | 2539 |
| CII373 | CDR | PRT | US20190085076; SEQ ID NO: 86 | 2540 |
| CII374 | CDR | PRT | W02016201389; SEQ ID NO: 86 | 2541 |
| CII375 | CDR | PRT | W02016201389; SEQ ID NO: 107 | 2542 |
| CII376 | CDR | PRT | W02016201389; SEQ ID NO: 43 | 2543 |
| CII377 | CDR | PRT | US20190085076; SEQ ID NO: 69 | 2544 |
| CII378 | CDR | PRT | W02016201389; SEQ ID NO: 69 | 2545 |
| CII379 | CDR | PRT | US20190085076; SEQ ID NO: 59 | 2546 |
| CII380 | CDR | PRT | W02016201389; SEQ ID NO: 59 | 2547 |
| CII381 | CDR | PRT | US20190085076; SEQ ID NO: 87 | 2548 |
| CII382 | CDR | PRT | W02016201389; SEQ ID NO: 87 | 2549 |
| CII383 | CDR | PRT | W02016201389; SEQ ID NO: 98 | 2550 |
| CII384 | CDR | PRT | W02016201389; SEQ ID NO: 96 | 2551 |
| CII385 | CDR | PRT | US20190085076; SEQ ID NO: 41 | 2552 |
| CII386 | CDR | PRT | W02016201389; SEQ ID NO: 41 | 2553 |
| CII387 | CDR | PRT | US20190085076; SEQ ID NO: 73 | 2554 |
| CII388 | CDR | PRT | W02016201389; SEQ ID NO: 73 | 2555 |
| CII389 | CDR | PRT | US20190085076; SEQ ID NO: 66 | 2556 |
| CII390 | CDR | PRT | W02016201389; SEQ ID NO: 66 | 2557 |
| CII391 | CDR | PRT | US20190085076; SEQ ID NO: 75 | 2558 |
| CII392 | CDR | PRT | W02016201389; SEQ ID NO: 75 | 2559 |
| CII393 | CDR | PRT | US20190085076; SEQ ID NO: 49 | 2560 |
| CII394 | CDR | PRT | W02016201389; SEQ ID NO: 49 | 2561 |
| CII395 | CDR | PRT | W02016201389; SEQ ID NO: 92 | 2562 |
| CII396 | CDR | PRT | US20190085076; SEQ ID NO: 74 | 2563 |
| CII397 | CDR | PRT | W02016201389; SEQ ID NO: 74 | 2564 |
| CII398 | CDR | PRT | US20190085076; SEQ ID NO: 55 | 2565 |
| CII399 | CDR | PRT | W02016201389; SEQ ID NO: 55 | 2566 |
| CII400 | CDR | PRT | US20190085076; SEQ ID NO: 95 | 2567 |
| CII401 | CDR | PRT | W02016201389; SEQ ID NO: 95 | 2568 |
| CII402 | CDR | PRT | US20190085076; SEQ ID NO: 48 | 2569 |
| CII403 | CDR | PRT | W02016201389; SEQ ID NO: 48 | 2570 |
| CII404 | CDR | PRT | US20190085076; SEQ ID NO: 53 | 2571 |
| CII405 | CDR | PRT | W02016201389; SEQ ID NO: 53 | 2572 |
| CII406 | CDR | PRT | W02016201389; SEQ ID NO: 39 | 2573 |
| CII407 | CDR | PRT | US20190085076; SEQ ID NO: 90 | 2574 |
| CII408 | CDR | PRT | W02016201389; SEQ ID NO: 90 | 2575 |
| CII409 | CDR | PRT | WO2016201388; SEQ ID NO: 16 | 2576 |
| CII410 | CDR | PRT | WO2016201388; SEQ ID NO: 190 | 2577 |
| CII411 | CDR | PRT | WO2016201388; SEQ ID NO: 12 | 2578 |
| CII412 | CDR | PRT | WO2016201388; SEQ ID NO: 68 | 2579 |
| CII413 | CDR | PRT | WO2016201388; SEQ ID NO: 11 | 2580 |
| CII414 | CDR | PRT | WO2016201388; SEQ ID NO: 67 | 2581 |
| CII415 | CDR | PRT | WO2019028283; SEQ ID NO: 130 | 2582 |
| CII416 | CDR | PRT | W02016201389; SEQ ID NO: 13 | 2583 |
| CII417 | CDR | PRT | US20190085076; SEQ ID NO: 15 | 2584 |
| CII418 | CDR | PRT | W02016201389; SEQ ID NO: 15 | 2585 |
| CII419 | CDR | PRT | US20190085076; SEQ ID NO: 23 | 2586 |
| CII420 | CDR | PRT | W02016201389; SEQ ID NO: 23 | 2587 |
| CII421 | CDR | PRT | W02016201389; SEQ ID NO: 14 | 2588 |
| CII422 | CDR | PRT | US20190085076; SEQ ID NO: 17 | 2589 |
| CII423 | CDR | PRT | W02016201389; SEQ ID NO: 17 | 2590 |
| CII424 | CDR | PRT | W02016201389; SEQ ID NO: 21 | 2591 |
| CII425 | CDR | PRT | WO2019028283; SEQ ID NO: 128 | 2592 |
| CII426 | CDR | PRT | WO2019028283; SEQ ID NO: 132 | 2593 |
| CII427 | CDR | PRT | WO2019028283; SEQ ID NO: 129 | 2594 |
| CII428 | CDR | PRT | US20190085076; SEQ ID NO: 11 | 2595 |
| CII429 | CDR | PRT | W02016201389; SEQ ID NO: 11 | 2596 |
| CII430 | CDR | PRT | US20190085076; SEQ ID NO: 12 | 2597 |
| CII431 | CDR | PRT | W02016201389; SEQ ID NO: 12 | 2598 |
| CII432 | CDR | PRT | US20190085076; SEQ ID NO: 20 | 2599 |
| CII433 | CDR | PRT | W02016201389; SEQ ID NO: 20 | 2600 |
| CII434 | CDR | PRT | US20190085076; SEQ ID NO: 9 | 2601 |
| CII435 | CDR | PRT | W02016201389; SEQ ID NO: 9 | 2602 |
| CII436 | CDR | PRT | WO2016201388; SEQ ID NO: 10 | 2603 |
| CII437 | CDR | PRT | WO2019028283; SEQ ID NO: 127 | 2604 |
| CII438 | CDR | PRT | WO2019028283; SEQ ID NO: 133 | 2605 |
| CII439 | CDR | PRT | WO2016201388; SEQ ID NO: 184 | 2606 |
| CII440 | CDR | PRT | US20190085076; SEQ ID NO: 18 | 2607 |
| CII441 | CDR | PRT | W02016201389; SEQ ID NO: 18 | 2608 |
| CII442 | CDR | PRT | WO2019028283; SEQ ID NO: 134 | 2609 |
| CII443 | CDR | PRT | WO2016201388; SEQ ID NO: 26 | 2610 |
| CII444 | CDR | PRT | US20190085076; SEQ ID NO: 30 | 2611 |
| CII445 | CDR | PRT | W02016201389; SEQ ID NO: 30 | 2612 |
| CII446 | CDR | PRT | WO2019028283; SEQ ID NO: 126 | 2613 |
| CII447 | CDR | PRT | WO2019028283; SEQ ID NO: 122 | 2614 |
| CII448 | CDR | PRT | WO2019028283; SEQ ID NO: 123 | 2615 |
| CII449 | CDR | PRT | W02016201389; SEQ ID NO: 157 | 2616 |
| CII450 | CDR | PRT | W02016201389; SEQ ID NO: 160 | 2617 |
| CII451 | CDR | PRT | US20190085076; SEQ ID NO: 152 | 2618 |
| CII452 | CDR | PRT | W02016201389; SEQ ID NO: 152 | 2619 |
| CII453 | CDR | PRT | US20190085076; SEQ ID NO: 148 | 2620 |
| CII454 | CDR | PRT | W02016201389; SEQ ID NO: 148 | 2621 |
| CII455 | CDR | PRT | US20190085076; SEQ ID NO: 141 | 2622 |
| CII456 | CDR | PRT | W02016201389; SEQ ID NO: 141 | 2623 |
| CII457 | CDR | PRT | WO2019028283; SEQ ID NO: 124 | 2624 |
| CII458 | CDR | PRT | WO2019028283; SEQ ID NO: 125 | 2625 |
| CII459 | CDR | PRT | WO2019028283; SEQ ID NO: 121 | 2626 |
| CII460 | CDR | PRT | WO2019028283; SEQ ID NO: 154 | 2627 |
| CII461 | CDR | PRT | WO2019028283; SEQ ID NO: 187 | 2628 |
| CII462 | CDR | PRT | W02016201389; SEQ ID NO: 138 | 2629 |
| CII463 | CDR | PRT | W02016201389; SEQ ID NO: 149 | 2630 |
| CII464 | CDR | PRT | WO2016201388; SEQ ID NO: 228 | 2631 |
| CII465 | CDR | PRT | WO2016201388; SEQ ID NO: 29 | 2632 |
| CII466 | CDR | PRT | WO2016201388; SEQ ID NO: 187 | 2633 |
| CII467 | CDR | PRT | WO2016201388; SEQ ID NO: 28 | 2634 |
| CII468 | CDR | PRT | WO2016201388; SEQ ID NO: 25 | 2635 |
| CII469 | CDR | PRT | WO2016201388; SEQ ID NO: 76 | 2636 |
| CII470 | CDR | PRT | WO2016201388; SEQ ID NO: 233 | 2637 |
| CII471 | CDR | PRT | WO2016201388; SEQ ID NO: 232 | 2638 |
| CII472 | CDR | PRT | US20190085076; SEQ ID NO: 150 | 2639 |
| CII473 | CDR | PRT | W02016201389; SEQ ID NO: 150 | 2640 |
| CII474 | CDR | PRT | W02016201389; SEQ ID NO: 155 | 2641 |
| CII475 | CDR | PRT | W02016201389; SEQ ID NO: 154 | 2642 |
| CII476 | CDR | PRT | US20190085076; SEQ ID NO: 144 | 2643 |
| CII477 | CDR | PRT | W02016201389; SEQ ID NO: 144 | 2644 |
| CII478 | CDR | PRT | WO2019028283; SEQ ID NO: 193 | 2645 |
| CII479 | CDR | PRT | W02016201389; SEQ ID NO: 25 | 2646 |
| CII480 | CDR | PRT | W02016201389; SEQ ID NO: 153 | 2647 |
| CII481 | CDR | PRT | W02016201389; SEQ ID NO: 143 | 2648 |
| CII482 | CDR | PRT | W02016201389; SEQ ID NO: 140 | 2649 |
| CII483 | CDR | PRT | W02016201389; SEQ ID NO: 147 | 2650 |
| CII484 | CDR | PRT | WO2019028283; SEQ ID NO: 157 | 2651 |
| CII485 | CDR | PRT | WO2019028283; SEQ ID NO: 155 | 2652 |
| CII486 | CDR | PRT | WO2019028283; SEQ ID NO: 188 | 2653 |
| CII487 | CDR | PRT | WO2019028283; SEQ ID NO: 140 | 2654 |
| CII488 | CDR | PRT | WO2019028283; SEQ ID NO: 137 | 2655 |
| CII489 | CDR | PRT | WO2019028283; SEQ ID NO: 144 | 2656 |
| CII490 | CDR | PRT | WO2019028283; SEQ ID NO: 135 | 2657 |
| CII491 | CDR | PRT | WO2019028283; SEQ ID NO: 138 | 2658 |
| CII492 | CDR | PRT | WO2019028283; SEQ ID NO: 145 | 2659 |
| CII493 | CDR | PRT | WO2019028283; SEQ ID NO: 142 | 2660 |
| CII494 | CDR | PRT | WO2019028283; SEQ ID NO: 141 | 2661 |
| CII495 | CDR | PRT | WO2019028283; SEQ ID NO: 139 | 2662 |
| CII496 | CDR | PRT | WO2019028283; SEQ ID NO: 191 | 2663 |
| CII497 | CDR | PRT | WO2019028283; SEQ ID NO: 143 | 2664 |
| CII498 | CDR | PRT | US20190085076; SEQ ID NO: 156 | 2665 |
| CII499 | CDR | PRT | W02016201389; SEQ ID NO: 156 | 2666 |
| CII500 | CDR | PRT | WO2016201388; SEQ ID NO: 69 | 2667 |
| CII501 | CDR | PRT | WO2016201388; SEQ ID NO: 13 | 2668 |
| CII502 | CDR | PRT | WO2016201388; SEQ ID NO: 20 | 2669 |
| CII503 | CDR | PRT | US20190085076; SEQ ID NO: 127 | 2670 |
| CII504 | CDR | PRT | W02016201389; SEQ ID NO: 127 | 2671 |
| CII505 | CDR | PRT | WO2016201388; SEQ ID NO: 19 | 2672 |
| CII506 | CDR | PRT | W02016201389; SEQ ID NO: 136 | 2673 |
| CII507 | CDR | PRT | W02016201389; SEQ ID NO: 118 | 2674 |
| CII508 | CDR | PRT | W02016201389; SEQ ID NO: 123 | 2675 |
| CII509 | CDR | PRT | WO2016201388; SEQ ID NO: 18 | 2676 |
| CII510 | CDR | PRT | WO2016201388; SEQ ID NO: 75 | 2677 |
| CII511 | CDR | PRT | W02016201389; SEQ ID NO: 135 | 2678 |
| CII512 | CDR | PRT | US20190085076; SEQ ID NO: 128 | 2679 |
| CII513 | CDR | PRT | W02016201389; SEQ ID NO: 128 | 2680 |
| CII514 | CDR | PRT | WO2019028283; SEQ ID NO: 136 | 2681 |
| CII515 | CDR | PRT | WO2019028283; SEQ ID NO: 156 | 2682 |
| CII516 | CDR | PRT | WO2019028283; SEQ ID NO: 189 | 2683 |
| CII517 | CDR | PRT | WO2018089794; SEQ ID NO: 15 | 2684 |
| CII518 | CDR | PRT | WO2018089790; SEQ ID NO: 11 | 2685 |
| CII519 | CDR | PRT | WO2018089795; SEQ ID NO: 16 | 2686 |
| CII520 | CDR | PRT | WO2018089793; SEQ ID NO: 14 | 2687 |
| CII521 | CDR | PRT | WO2018089792; SEQ ID NO: 13 | 2688 |
| CII522 | CDR | PRT | WO2018089791; SEQ ID NO: 12 | 2689 |
| CII523 | CDR | PRT | US20180333503; SEQ ID NO: 9 | 2690 |
| CII524 | CDR | PRT | US20180333503; SEQ ID NO: 6 | 2691 |
| CII525 | CDR | PRT | US20180333503; SEQ ID NO: 8 | 2692 |
| CII526 | CDR | PRT | US20180333503; SEQ ID NO: 7 | 2693 |
| CII527 | CDR | PRT | US20180333503; SEQ ID NO: 11 | 2694 |
| CII528 | CDR | PRT | US20180333503; SEQ ID NO: 10 | 2695 |
| CII529 | CDR | PRT | US20180333503; SEQ ID NO: 12 | 2696 |
| CII530 | CDR | PRT | US20190117769; SEQ ID NO: 18 | 2697 |
| CII531 | CDR | PRT | US20190117769; SEQ ID NO: 17 | 2698 |
| CII532 | CDR | PRT | US20190117769; SEQ ID NO: 20 | 2699 |
| CII533 | CDR | PRT | US20190117769; SEQ ID NO: 19 | 2700 |
| CII534 | CDR | PRT | US20190117769; SEQ ID NO: 22 | 2701 |
| CII535 | CDR | PRT | US20190117769; SEQ ID NO: 21 | 2702 |
| CII536 | CDR | PRT | US20180333503; SEQ ID NO: 21 | 2703 |
| CII537 | CDR | PRT | US20180333503; SEQ ID NO: 24 | 2704 |
| CII538 | CDR | PRT | US20180333503; SEQ ID NO: 20 | 2705 |
| CII539 | CDR | PRT | US20180333503; SEQ ID NO: 25 | 2706 |
| CII540 | CDR | PRT | US20180333503; SEQ ID NO: 23 | 2707 |
| CII541 | CDR | PRT | US20180333503; SEQ ID NO: 22 | 2708 |
| CII542 | CDR | PRT | WO2018089786; SEQ ID NO: 7 | 2709 |
| CII543 | CDR | PRT | WO2018089784; SEQ ID NO: 5 | 2710 |
| CII544 | CDR | PRT | WO2018089783; SEQ ID NO: 4 | 2711 |
| CII545 | CDR | PRT | WO2018089787; SEQ ID NO: 8 | 2712 |
| CII546 | CDR | PRT | WO2018089785; SEQ ID NO: 6 | 2713 |
| CII547 | CDR | PRT | WO2018089782; SEQ ID NO: 3 | 2714 |
| CII548 | CDR | PRT | WO2018213316; SEQ ID NO: 20 | 2715 |
| CII549 | CDR | PRT | WO2018213316; SEQ ID NO: 28 | 2716 |
| CII550 | CDR | PRT | WO2018213316; SEQ ID NO: 25 | 2717 |
| CII551 | CDR | PRT | WO2018213316; SEQ ID NO: 48 | 2718 |
| CII552 | CDR | PRT | WO2018213316; SEQ ID NO: 52 | 2719 |
| CII553 | CDR | PRT | WO2018213316; SEQ ID NO: 45 | 2720 |
| CII554 | CDR | PRT | WO2018213316; SEQ ID NO: 34 | 2721 |
| CII555 | CDR | PRT | WO2018213316; SEQ ID NO: 31 | 2722 |
| CII556 | CDR | PRT | WO2018213316; SEQ ID NO: 27 | 2723 |
| CII557 | CDR | PRT | WO2018213316; SEQ ID NO: 29 | 2724 |
| CII558 | CDR | PRT | WO2018213316; SEQ ID NO: 30 | 2725 |
| CII559 | CDR | PRT | WO2018213316; SEQ ID NO: 33 | 2726 |
| CII560 | CDR | PRT | WO2018213316; SEQ ID NO: 32 | 2727 |
| CII561 | CDR | PRT | WO2018213316; SEQ ID NO: 50 | 2728 |
| CII562 | CDR | PRT | WO2018213316; SEQ ID NO: 6 | 2729 |
| CII563 | CDR | PRT | WO2018213316; SEQ ID NO: 4 | 2730 |
| CII564 | CDR | PRT | WO2018213316; SEQ ID NO: 11 | 2731 |
| CII565 | CDR | PRT | WO2018213316; SEQ ID NO: 5 | 2732 |
| CII566 | CDR | PRT | WO2018213316; SEQ ID NO: 47 | 2733 |
| CII567 | CDR | PRT | WO2018213316; SEQ ID NO: 19 | 2734 |
| CII568 | CDR | PRT | WO2018213316; SEQ ID NO: 16 | 2735 |
| CII569 | CDR | PRT | WO2018213316; SEQ ID NO: 17 | 2736 |
| CII570 | CDR | PRT | WO2018213316; SEQ ID NO: 49 | 2737 |
| CII571 | CDR | PRT | WO2018213316; SEQ ID NO: 51 | 2738 |
| CII572 | CDR | PRT | WO2018213316; SEQ ID NO: 54 | 2739 |
| CII573 | CDR | PRT | WO2018213316; SEQ ID NO: 22 | 2740 |
| CII574 | CDR | PRT | WO2018213316; SEQ ID NO: 26 | 2741 |
| CII575 | CDR | PRT | WO2018213316; SEQ ID NO: 21 | 2742 |
| CII576 | CDR | PRT | WO2018213316; SEQ ID NO: 23 | 2743 |
| CII577 | CDR | PRT | WO2018213316; SEQ ID NO: 24 | 2744 |
| CII578 | CDR | PRT | WO2018213316; SEQ ID NO: 7 | 2745 |
| CII579 | CDR | PRT | WO2018213316; SEQ ID NO: 8 | 2746 |
| CII580 | CDR | PRT | WO2018213316; SEQ ID NO: 9 | 2747 |
| CII581 | CDR | PRT | WO2018213316; SEQ ID NO: 10 | 2748 |
| CII582 | CDR | PRT | WO2018213316; SEQ ID NO: 3 | 2749 |
| CII583 | CDR | PRT | WO2018213316; SEQ ID NO: 15 | 2750 |
| CII584 | CDR | PRT | WO2018213316; SEQ ID NO: 12 | 2751 |
| CII585 | CDR | PRT | WO2018213316; SEQ ID NO: 18 | 2752 |
| CII586 | CDR | PRT | WO2018213316; SEQ ID NO: 14 | 2753 |
| CII587 | CDR | PRT | WO2018213316; SEQ ID NO: 13 | 2754 |
| CII588 | CDR | PRT | WO2018213316; SEQ ID NO: 41 | 2755 |
| CII589 | CDR | PRT | WO2018213316; SEQ ID NO: 42 | 2756 |
| CII590 | CDR | PRT | WO2018213316; SEQ ID NO: 40 | 2757 |
| CII591 | CDR | PRT | WO2018213316; SEQ ID NO: 38 | 2758 |
| CII592 | CDR | PRT | WO2018213316; SEQ ID NO: 44 | 2759 |
| CII593 | CDR | PRT | WO2018213316; SEQ ID NO: 37 | 2760 |
| CII594 | CDR | PRT | WO2018213316; SEQ ID NO: 36 | 2761 |
| CII595 | CDR | PRT | WO2018213316; SEQ ID NO: 43 | 2762 |
| CII596 | CDR | PRT | WO2018213316; SEQ ID NO: 35 | 2763 |
| CII597 | CDR | PRT | WO2018213316; SEQ ID NO: 39 | 2764 |
| CII598 | CDR | PRT | WO2018213316; SEQ ID NO: 53 | 2765 |
| CII599 | CDR | PRT | WO2018213316; SEQ ID NO: 46 | 2766 |
| CII600 | CDR | PRT | WO2017040301; SEQ ID NO: 29 | 2767 |
| CII601 | CDR | PRT | WO2017040301; SEQ ID NO: 81 | 2768 |
| CII602 | CDR | PRT | WO2017040301; SEQ ID NO: 83 | 2769 |
| CII603 | CDR | PRT | WO2017040301; SEQ ID NO: 78 | 2770 |
| CII604 | CDR | PRT | WO2017040301; SEQ ID NO: 76 | 2771 |
| CII605 | CDR | PRT | WO2017040301; SEQ ID NO: 74 | 2772 |
| CII606 | CDR | PRT | WO2017040301; SEQ ID NO: 72 | 2773 |
| CII607 | CDR | PRT | WO2017040301; SEQ ID NO: 75 | 2774 |
| CII608 | CDR | PRT | WO2017040301; SEQ ID NO: 77 | 2775 |
| CII609 | CDR | PRT | WO2017040301; SEQ ID NO: 82 | 2776 |
| CII610 | CDR | PRT | WO2017040301; SEQ ID NO: 73 | 2777 |
| CII611 | CDR | PRT | WO2017040301; SEQ ID NO: 80 | 2778 |
| CII612 | CDR | PRT | WO2017040301; SEQ ID NO: 22 | 2779 |
| CII613 | CDR | PRT | WO2017040301; SEQ ID NO: 27 | 2780 |
| CII614 | CDR | PRT | WO2017040301; SEQ ID NO: 483 | 2781 |
| CII615 | CDR | PRT | WO2017040301; SEQ ID NO: 28 | 2782 |
| CII616 | CDR | PRT | WO2017040301; SEQ ID NO: 32 | 2783 |
| CII617 | CDR | PRT | WO2017040301; SEQ ID NO: 53 | 2784 |
| CII618 | CDR | PRT | WO2017040301; SEQ ID NO: 47 | 2785 |
| CII619 | CDR | PRT | WO2017040301; SEQ ID NO: 55 | 2786 |
| CII620 | CDR | PRT | WO2017040301; SEQ ID NO: 54 | 2787 |
| CII621 | CDR | PRT | WO2017040301; SEQ ID NO: 48 | 2788 |
| CII622 | CDR | PRT | WO2017040301; SEQ ID NO: 58 | 2789 |
| CII623 | CDR | PRT | WO2017040301; SEQ ID NO: 23 | 2790 |
| CII624 | CDR | PRT | WO2017040301; SEQ ID NO: 479 | 2791 |
| CII625 | CDR | PRT | WO2017040301; SEQ ID NO: 39 | 2792 |
| CII626 | CDR | PRT | WO2017040301; SEQ ID NO: 36 | 2793 |
| CII627 | CDR | PRT | WO2017040301; SEQ ID NO: 59 | 2794 |
| CII628 | CDR | PRT | WO2017040301; SEQ ID NO: 62 | 2795 |
| CII629 | CDR | PRT | WO2017040301; SEQ ID NO: 64 | 2796 |
| CII630 | CDR | PRT | WO2017040301; SEQ ID NO: 69 | 2797 |
| CII631 | CDR | PRT | WO2017040301; SEQ ID NO: 63 | 2798 |
| CII632 | CDR | PRT | WO2017040301; SEQ ID NO: 65 | 2799 |
| CII633 | CDR | PRT | WO2017040301; SEQ ID NO: 70 | 2800 |
| CII634 | CDR | PRT | WO2017040301; SEQ ID NO: 60 | 2801 |
| CII635 | CDR | PRT | WO2017040301; SEQ ID NO: 10 | 2802 |
| CII636 | CDR | PRT | WO2017040301; SEQ ID NO: 13 | 2803 |
| CII637 | CDR | PRT | WO2017040301; SEQ ID NO: 24 | 2804 |
| CII638 | CDR | PRT | WO2017040301; SEQ ID NO: 20 | 2805 |
| CII639 | CDR | PRT | WO2017040301; SEQ ID NO: 30 | 2806 |
| CII640 | CDR | PRT | WO2017040301; SEQ ID NO: 25 | 2807 |
| CII641 | CDR | PRT | WO2017040301; SEQ ID NO: 16 | 2808 |
| CII642 | CDR | PRT | WO2017040301; SEQ ID NO: 68 | 2809 |
| CII643 | CDR | PRT | WO2017040301; SEQ ID NO: 31 | 2810 |
| CII644 | CDR | PRT | WO2017040301; SEQ ID NO: 45 | 2811 |
| CII645 | CDR | PRT | WO2017040301; SEQ ID NO: 34 | 2812 |
| CII646 | CDR | PRT | WO2017040301; SEQ ID NO: 41 | 2813 |
| CII647 | CDR | PRT | WO2017040301; SEQ ID NO: 44 | 2814 |
| CII648 | CDR | PRT | WO2017040301; SEQ ID NO: 478 | 2815 |
| CII649 | CDR | PRT | WO2017040301; SEQ ID NO: 42 | 2816 |
| CII650 | CDR | PRT | WO2017040301; SEQ ID NO: 35 | 2817 |
| CII651 | CDR | PRT | WO2017040301; SEQ ID NO: 40 | 2818 |
| CII652 | CDR | PRT | WO2017040301; SEQ ID NO: 476 | 2819 |
| CII653 | CDR | PRT | WO2017040301; SEQ ID NO: 18 | 2820 |
| CII654 | CDR | PRT | WO2017040301; SEQ ID NO: 19 | 2821 |
| CII655 | CDR | PRT | WO2017040301; SEQ ID NO: 8 | 2822 |
| CII656 | CDR | PRT | WO2017040301; SEQ ID NO: 15 | 2823 |
| CII657 | CDR | PRT | WO2017040301; SEQ ID NO: 482 | 2824 |
| CII658 | CDR | PRT | WO2017040301; SEQ ID NO: 12 | 2825 |
| CII659 | CDR | PRT | WO2017040301; SEQ ID NO: 11 | 2826 |
| CII660 | CDR | PRT | WO2017040301; SEQ ID NO: 17 | 2827 |
| CII661 | CDR | PRT | WO2017040301; SEQ ID NO: 7 | 2828 |
| CII662 | CDR | PRT | WO2017040301; SEQ ID NO: 9 | 2829 |
| CII663 | CDR | PRT | WO2017040301; SEQ ID NO: 14 | 2830 |
| CII664 | CDR | PRT | WO2017040301; SEQ ID NO: 26 | 2831 |
| CII665 | CDR | PRT | WO2017040301; SEQ ID NO: 46 | 2832 |
| CII666 | CDR | PRT | WO2017040301; SEQ ID NO: 49 | 2833 |
| CII667 | CDR | PRT | WO2017040301; SEQ ID NO: 51 | 2834 |
| CII668 | CDR | PRT | WO2017040301; SEQ ID NO: 56 | 2835 |
| CII669 | CDR | PRT | WO2017040301; SEQ ID NO: 33 | 2836 |
| CII670 | CDR | PRT | WO2017040301; SEQ ID NO: 37 | 2837 |
| CII671 | CDR | PRT | WO2017040301; SEQ ID NO: 38 | 2838 |
| CII672 | CDR | PRT | WO2017040301; SEQ ID NO: 43 | 2839 |
| CII673 | CDR | PRT | WO2017040301; SEQ ID NO: 79 | 2840 |
| CII674 | CDR | PRT | WO2017040301; SEQ ID NO: 71 | 2841 |
| CII675 | CDR | PRT | WO2017040301; SEQ ID NO: 67 | 2842 |
| CII676 | CDR | PRT | WO2017040301; SEQ ID NO: 61 | 2843 |
| CII677 | CDR | PRT | WO2017040301; SEQ ID NO: 481 | 2844 |
| CII678 | CDR | PRT | WO2017040301; SEQ ID NO: 66 | 2845 |
| CII679 | CDR | PRT | WO2017040301; SEQ ID NO: 84 | 2846 |
| CII680 | CDR | PRT | WO2017040301; SEQ ID NO: 480 | 2847 |
| CII681 | CDR | PRT | WO2017040301; SEQ ID NO: 57 | 2848 |
| CII682 | CDR | PRT | WO2017040301; SEQ ID NO: 21 | 2849 |
| CII683 | CDR | PRT | WO2017040301; SEQ ID NO: 50 | 2850 |
| CII684 | CDR | PRT | WO2017040301; SEQ ID NO: 52 | 2851 |
| CII685 | CDR | PRT | WO2017040301; SEQ ID NO: 477 | 2852 |
| CII686 | CDR | PRT | WO2017075432; SEQ ID NO: 29 | 2853 |
| CII687 | CDR | PRT | WO2017075432; SEQ ID NO: 17 | 2854 |
| CII688 | CDR | PRT | WO2017075432; SEQ ID NO: 27 | 2855 |
| CII689 | CDR | PRT | WO2017075432; SEQ ID NO: 13 | 2856 |
| CII690 | CDR | PRT | WO2017075432; SEQ ID NO: 19 | 2857 |
| CII691 | CDR | PRT | WO2017075432; SEQ ID NO: 179 | 2858 |
| CII692 | CDR | PRT | WO2017075432; SEQ ID NO: 182 | 2859 |
| CII693 | CDR | PRT | WO2017075432; SEQ ID NO: 178 | 2860 |
| CII694 | CDR | PRT | WO2017075432; SEQ ID NO: 9 | 2861 |
| CII695 | CDR | PRT | WO2017075432; SEQ ID NO: 25 | 2862 |
| CII696 | CDR | PRT | WO2017075432; SEQ ID NO: 24 | 2863 |
| CII697 | CDR | PRT | WO2017075432; SEQ ID NO: 23 | 2864 |
| CII698 | CDR | PRT | WO2017075432; SEQ ID NO: 7 | 2865 |
| CII699 | CDR | PRT | WO2017075432; SEQ ID NO: 8 | 2866 |
| CII700 | CDR | PRT | WO2017075432; SEQ ID NO: 174 | 2867 |
| CII701 | CDR | PRT | WO2017075432; SEQ ID NO: 18 | 2868 |
| CII702 | CDR | PRT | WO2017075432; SEQ ID NO: 16 | 2869 |
| CII703 | CDR | PRT | WO2017075432; SEQ ID NO: 14 | 2870 |
| CII704 | CDR | PRT | WO2017075432; SEQ ID NO: 11 | 2871 |
| CII705 | CDR | PRT | WO2017075432; SEQ ID NO: 12 | 2872 |
| CII706 | CDR | PRT | WO2017075432; SEQ ID NO: 177 | 2873 |
| CII707 | CDR | PRT | WO2017075432; SEQ ID NO: 173 | 2874 |
| CII708 | CDR | PRT | WO2017075432; SEQ ID NO: 175 | 2875 |
| CII709 | CDR | PRT | WO2017075432; SEQ ID NO: 6 | 2876 |
| CII710 | CDR | PRT | WO2017075432; SEQ ID NO: 176 | 2877 |
| CII711 | CDR | PRT | WO2017075432; SEQ ID NO: 172 | 2878 |
| CII712 | CDR | PRT | WO2017075432; SEQ ID NO: 10 | 2879 |
| CII713 | CDR | PRT | WO2017075432; SEQ ID NO: 181 | 2880 |
| CII714 | CDR | PRT | WO2017075432; SEQ ID NO: 28 | 2881 |
| CII715 | CDR | PRT | WO2017075432; SEQ ID NO: 22 | 2882 |
| CII716 | CDR | PRT | WO2017075432; SEQ ID NO: 26 | 2883 |
| CII717 | CDR | PRT | WO2017075432; SEQ ID NO: 15 | 2884 |
| CII718 | CDR | PRT | WO2017075432; SEQ ID NO: 20 | 2885 |
| CII719 | CDR | PRT | WO2017075432; SEQ ID NO: 180 | 2886 |
| CII720 | CDR | PRT | WO2017075432; SEQ ID NO: 21 | 2887 |
| CII721 | CDR | PRT | WO2017075432; SEQ ID NO: 183 | 2888 |
| CII722 | CDR | PRT | WO2018107058; SEQ ID NO: 17 | 2889 |
| CII723 | CDR | PRT | WO2018107058; SEQ ID NO: 9 | 2890 |
| CII724 | CDR | PRT | WO2018107058; SEQ ID NO: 15 | 2891 |
| CII725 | CDR | PRT | WO2018107058; SEQ ID NO: 7 | 2892 |
| CII726 | CDR | PRT | WO2018107058; SEQ ID NO: 16 | 2893 |
| CII727 | CDR | PRT | WO2018107058; SEQ ID NO: 11 | 2894 |
| CII728 | CDR | PRT | WO2018107058; SEQ ID NO: 8 | 2895 |
| CII729 | CDR | PRT | WO2018107058; SEQ ID NO: 14 | 2896 |
| CII730 | CDR | PRT | WO2018107058; SEQ ID NO: 6 | 2897 |
| CII731 | CDR | PRT | WO2018107058; SEQ ID NO: 12 | 2898 |
| CII732 | CDR | PRT | WO2018107058; SEQ ID NO: 10 | 2899 |
| CII733 | CDR | PRT | WO2018107058; SEQ ID NO: 13 | 2900 |
| CII734 | CDR | PRT | US20190085084; SEQ ID NO: 32 | 2901 |
| CII735 | CDR | PRT | US20190085084; SEQ ID NO: 32 | 2902 |
| CII736 | CDR | PRT | WO2016164637; SEQ ID NO: 32 | 2903 |
| CII737 | CDR | PRT | US20190085084; SEQ ID NO: 27 | 2904 |
| CII738 | CDR | PRT | US20190085084; SEQ ID NO: 27 | 2905 |
| CII739 | CDR | PRT | WO2016164637; SEQ ID NO: 27 | 2906 |
| CII740 | CDR | PRT | US20190085084; SEQ ID NO: 164 | 2907 |
| CII741 | CDR | PRT | US20190085084; SEQ ID NO: 164 | 2908 |
| CII742 | CDR | PRT | WO2016164637; SEQ ID NO: 164 | 2909 |
| CII743 | CDR | PRT | US20190085084; SEQ ID NO: 163 | 2910 |
| CII744 | CDR | PRT | US20190085084; SEQ ID NO: 163 | 2911 |
| CII745 | CDR | PRT | WO2016164637; SEQ ID NO: 163 | 2912 |
| CII746 | CDR | PRT | US20190085084; SEQ ID NO: 588 | 2913 |
| CII747 | CDR | PRT | WO2016164637; SEQ ID NO: 588 | 2914 |
| CII748 | CDR | PRT | US20190085084; SEQ ID NO: 225 | 2915 |
| CII749 | CDR | PRT | US20190085084; SEQ ID NO: 225 | 2916 |
| CII750 | CDR | PRT | WO2016164637; SEQ ID NO: 225 | 2917 |
| CII751 | CDR | PRT | US20190085084; SEQ ID NO: 200 | 2918 |
| CII752 | CDR | PRT | US20190085084; SEQ ID NO: 200 | 2919 |
| CII753 | CDR | PRT | WO2016164637; SEQ ID NO: 200 | 2920 |
| CII754 | CDR | PRT | US20190085084; SEQ ID NO: 216 | 2921 |
| CII755 | CDR | PRT | US20190085084; SEQ ID NO: 216 | 2922 |
| CII756 | CDR | PRT | WO2016164637; SEQ ID NO: 216 | 2923 |
| CII757 | CDR | PRT | US20190085084; SEQ ID NO: 213 | 2924 |
| CII758 | CDR | PRT | US20190085084; SEQ ID NO: 213 | 2925 |
| CII759 | CDR | PRT | WO2016164637; SEQ ID NO: 213 | 2926 |
| CII760 | CDR | PRT | US20190085084; SEQ ID NO: 199 | 2927 |
| CII761 | CDR | PRT | US20190085084; SEQ ID NO: 199 | 2928 |
| CII762 | CDR | PRT | WO2016164637; SEQ ID NO: 199 | 2929 |
| CII763 | CDR | PRT | US20190085084; SEQ ID NO: 206 | 2930 |
| CII764 | CDR | PRT | US20190085084; SEQ ID NO: 206 | 2931 |
| CII765 | CDR | PRT | WO2016164637; SEQ ID NO: 206 | 2932 |
| CII766 | CDR | PRT | US20190085084; SEQ ID NO: 208 | 2933 |
| CII767 | CDR | PRT | US20190085084; SEQ ID NO: 208 | 2934 |
| CII768 | CDR | PRT | WO2016164637; SEQ ID NO: 208 | 2935 |
| CII769 | CDR | PRT | US20190085084; SEQ ID NO: 218 | 2936 |
| CII770 | CDR | PRT | US20190085084; SEQ ID NO: 218 | 2937 |
| CII771 | CDR | PRT | WO2016164637; SEQ ID NO: 218 | 2938 |
| CII772 | CDR | PRT | US20190085084; SEQ ID NO: 217 | 2939 |
| CII773 | CDR | PRT | US20190085084; SEQ ID NO: 217 | 2940 |
| CII774 | CDR | PRT | WO2016164637; SEQ ID NO: 217 | 2941 |
| CII775 | CDR | PRT | US20190085084; SEQ ID NO: 236 | 2942 |
| CII776 | CDR | PRT | US20190085084; SEQ ID NO: 236 | 2943 |
| CII777 | CDR | PRT | WO2016164637; SEQ ID NO: 236 | 2944 |
| CII778 | CDR | PRT | US20190085084; SEQ ID NO: 224 | 2945 |
| CII779 | CDR | PRT | US20190085084; SEQ ID NO: 224 | 2946 |
| CII780 | CDR | PRT | WO2016164637; SEQ ID NO: 224 | 2947 |
| CII781 | CDR | PRT | US20190085084; SEQ ID NO: 193 | 2948 |
| CII782 | CDR | PRT | US20190085084; SEQ ID NO: 193 | 2949 |
| CII783 | CDR | PRT | WO2016164637; SEQ ID NO: 193 | 2950 |
| CII784 | CDR | PRT | US20190085084; SEQ ID NO: 188 | 2951 |
| CII785 | CDR | PRT | US20190085084; SEQ ID NO: 188 | 2952 |
| CII786 | CDR | PRT | WO2016164637; SEQ ID NO: 188 | 2953 |
| CII787 | CDR | PRT | US20190085084; SEQ ID NO: 556 | 2954 |
| CII788 | CDR | PRT | US20190085084; SEQ ID NO: 556 | 2955 |
| CII789 | CDR | PRT | WO2016164637; SEQ ID NO: 556 | 2956 |
| CII790 | CDR | PRT | US20190085084; SEQ ID NO: 559 | 2957 |
| CII791 | CDR | PRT | US20190085084; SEQ ID NO: 559 | 2958 |
| CII792 | CDR | PRT | WO2016164637; SEQ ID NO: 559 | 2959 |
| CII793 | CDR | PRT | US20190085084; SEQ ID NO: 186 | 2960 |
| CII794 | CDR | PRT | US20190085084; SEQ ID NO: 186 | 2961 |
| CII795 | CDR | PRT | WO2016164637; SEQ ID NO: 186 | 2962 |
| CII796 | CDR | PRT | US20190085084; SEQ ID NO: 184 | 2963 |
| CII797 | CDR | PRT | US20190085084; SEQ ID NO: 184 | 2964 |
| CII798 | CDR | PRT | WO2016164637; SEQ ID NO: 184 | 2965 |
| CII799 | CDR | PRT | US20190085084; SEQ ID NO: 557 | 2966 |
| CII800 | CDR | PRT | US20190085084; SEQ ID NO: 557 | 2967 |
| CII801 | CDR | PRT | WO2016164637; SEQ ID NO: 557 | 2968 |
| CII802 | CDR | PRT | US20190085084; SEQ ID NO: 563 | 2969 |
| CII803 | CDR | PRT | US20190085084; SEQ ID NO: 563 | 2970 |
| CII804 | CDR | PRT | WO2016164637; SEQ ID NO: 563 | 2971 |
| CII805 | CDR | PRT | US20190085084; SEQ ID NO: 555 | 2972 |
| CII806 | CDR | PRT | US20190085084; SEQ ID NO: 555 | 2973 |
| CII807 | CDR | PRT | WO2016164637; SEQ ID NO: 555 | 2974 |
| CII808 | CDR | PRT | US20190085084; SEQ ID NO: 540 | 2975 |
| CII809 | CDR | PRT | US20190085084; SEQ ID NO: 540 | 2976 |
| CII810 | CDR | PRT | WO2016164637; SEQ ID NO: 540 | 2977 |
| CII811 | CDR | PRT | US20190085084; SEQ ID NO: 541 | 2978 |
| CII812 | CDR | PRT | US20190085084; SEQ ID NO: 541 | 2979 |
| CII813 | CDR | PRT | WO2016164637; SEQ ID NO: 541 | 2980 |
| CII814 | CDR | PRT | US20190085084; SEQ ID NO: 542 | 2981 |
| CII815 | CDR | PRT | US20190085084; SEQ ID NO: 542 | 2982 |
| CII816 | CDR | PRT | WO2016164637; SEQ ID NO: 542 | 2983 |
| CII817 | CDR | PRT | US20190085084; SEQ ID NO: 190 | 2984 |
| CII818 | CDR | PRT | US20190085084; SEQ ID NO: 190 | 2985 |
| CII819 | CDR | PRT | WO2016164637; SEQ ID NO: 190 | 2986 |
| CII820 | CDR | PRT | US20190085084; SEQ ID NO: 558 | 2987 |
| CII821 | CDR | PRT | US20190085084; SEQ ID NO: 558 | 2988 |
| CII822 | CDR | PRT | WO2016164637; SEQ ID NO: 558 | 2989 |
| CII823 | CDR | PRT | US20190085084; SEQ ID NO: 560 | 2990 |
| CII824 | CDR | PRT | US20190085084; SEQ ID NO: 560 | 2991 |
| CII825 | CDR | PRT | WO2016164637; SEQ ID NO: 560 | 2992 |
| CII826 | CDR | PRT | US20190085084; SEQ ID NO: 543 | 2993 |
| CII827 | CDR | PRT | US20190085084; SEQ ID NO: 543 | 2994 |
| CII828 | CDR | PRT | WO2016164637; SEQ ID NO: 543 | 2995 |
| CII829 | CDR | PRT | US20190085084; SEQ ID NO: 554 | 2996 |
| CII830 | CDR | PRT | US20190085084; SEQ ID NO: 554 | 2997 |
| CII831 | CDR | PRT | WO2016164637; SEQ ID NO: 554 | 2998 |
| CII832 | CDR | PRT | US20190085084; SEQ ID NO: 544 | 2999 |
| CII833 | CDR | PRT | US20190085084; SEQ ID NO: 544 | 3000 |
| CII834 | CDR | PRT | WO2016164637; SEQ ID NO: 544 | 3001 |
| CII835 | CDR | PRT | US20190085084; SEQ ID NO: 553 | 3002 |
| CII836 | CDR | PRT | US20190085084; SEQ ID NO: 553 | 3003 |
| CII837 | CDR | PRT | WO2016164637; SEQ ID NO: 553 | 3004 |
| CII838 | CDR | PRT | US20190085084; SEQ ID NO: 545 | 3005 |
| CII839 | CDR | PRT | US20190085084; SEQ ID NO: 545 | 3006 |
| CII840 | CDR | PRT | WO2016164637; SEQ ID NO: 545 | 3007 |
| CII841 | CDR | PRT | US20190085084; SEQ ID NO: 546 | 3008 |
| CII842 | CDR | PRT | US20190085084; SEQ ID NO: 546 | 3009 |
| CII843 | CDR | PRT | WO2016164637; SEQ ID NO: 546 | 3010 |
| CII844 | CDR | PRT | US20190085084; SEQ ID NO: 562 | 3011 |
| CII845 | CDR | PRT | US20190085084; SEQ ID NO: 562 | 3012 |
| CII846 | CDR | PRT | WO2016164637; SEQ ID NO: 562 | 3013 |
| CII847 | CDR | PRT | US20190085084; SEQ ID NO: 552 | 3014 |
| CII848 | CDR | PRT | US20190085084; SEQ ID NO: 552 | 3015 |
| CII849 | CDR | PRT | WO2016164637; SEQ ID NO: 552 | 3016 |
| CII850 | CDR | PRT | US20190085084; SEQ ID NO: 547 | 3017 |
| CII851 | CDR | PRT | US20190085084; SEQ ID NO: 547 | 3018 |
| CII852 | CDR | PRT | WO2016164637; SEQ ID NO: 547 | 3019 |
| CII853 | CDR | PRT | US20190085084; SEQ ID NO: 548 | 3020 |
| CII854 | CDR | PRT | US20190085084; SEQ ID NO: 548 | 3021 |
| CII855 | CDR | PRT | WO2016164637; SEQ ID NO: 548 | 3022 |
| CII856 | CDR | PRT | US20190085084; SEQ ID NO: 564 | 3023 |
| CII857 | CDR | PRT | US20190085084; SEQ ID NO: 564 | 3024 |
| CII858 | CDR | PRT | WO2016164637; SEQ ID NO: 564 | 3025 |
| CII859 | CDR | PRT | US20190085084; SEQ ID NO: 549 | 3026 |
| CII860 | CDR | PRT | US20190085084; SEQ ID NO: 549 | 3027 |
| CII861 | CDR | PRT | WO2016164637; SEQ ID NO: 549 | 3028 |
| CII862 | CDR | PRT | US20190085084; SEQ ID NO: 561 | 3029 |
| CII863 | CDR | PRT | US20190085084; SEQ ID NO: 561 | 3030 |
| CII864 | CDR | PRT | WO2016164637; SEQ ID NO: 561 | 3031 |
| CII865 | CDR | PRT | US20190085084; SEQ ID NO: 241 | 3032 |
| CII866 | CDR | PRT | US20190085084; SEQ ID NO: 241 | 3033 |
| CII867 | CDR | PRT | WO2016164637; SEQ ID NO: 241 | 3034 |
| CII868 | CDR | PRT | US20190085084; SEQ ID NO: 223 | 3035 |
| CII869 | CDR | PRT | US20190085084; SEQ ID NO: 223 | 3036 |
| CII870 | CDR | PRT | WO2016164637; SEQ ID NO: 223 | 3037 |
| CII871 | CDR | PRT | US20190085084; SEQ ID NO: 246 | 3038 |
| CII872 | CDR | PRT | US20190085084; SEQ ID NO: 246 | 3039 |
| CII873 | CDR | PRT | WO2016164637; SEQ ID NO: 246 | 3040 |
| CII874 | CDR | PRT | US20190085084; SEQ ID NO: 244 | 3041 |
| CII875 | CDR | PRT | US20190085084; SEQ ID NO: 244 | 3042 |
| CII876 | CDR | PRT | WO2016164637; SEQ ID NO: 244 | 3043 |
| CII877 | CDR | PRT | US20190085084; SEQ ID NO: 201 | 3044 |
| CII878 | CDR | PRT | US20190085084; SEQ ID NO: 201 | 3045 |
| CII879 | CDR | PRT | WO2016164637; SEQ ID NO: 201 | 3046 |
| CII880 | CDR | PRT | US20190085084; SEQ ID NO: 238 | 3047 |
| CII881 | CDR | PRT | US20190085084; SEQ ID NO: 238 | 3048 |
| CII882 | CDR | PRT | WO2016164637; SEQ ID NO: 238 | 3049 |
| CII883 | CDR | PRT | US20190085084; SEQ ID NO: 237 | 3050 |
| CII884 | CDR | PRT | US20190085084; SEQ ID NO: 237 | 3051 |
| CII885 | CDR | PRT | WO2016164637; SEQ ID NO: 237 | 3052 |
| CII886 | CDR | PRT | US20190085084; SEQ ID NO: 195 | 3053 |
| CII887 | CDR | PRT | US20190085084; SEQ ID NO: 195 | 3054 |
| CII888 | CDR | PRT | WO2016164637; SEQ ID NO: 195 | 3055 |
| CII889 | CDR | PRT | US20190085084; SEQ ID NO: 196 | 3056 |
| CII890 | CDR | PRT | US20190085084; SEQ ID NO: 196 | 3057 |
| CII891 | CDR | PRT | WO2016164637; SEQ ID NO: 196 | 3058 |
| CII892 | CDR | PRT | US20190085084; SEQ ID NO: 252 | 3059 |
| CII893 | CDR | PRT | US20190085084; SEQ ID NO: 252 | 3060 |
| CII894 | CDR | PRT | WO2016164637; SEQ ID NO: 252 | 3061 |
| CII895 | CDR | PRT | US20190085084; SEQ ID NO: 222 | 3062 |
| CII896 | CDR | PRT | US20190085084; SEQ ID NO: 222 | 3063 |
| CII897 | CDR | PRT | WO2016164637; SEQ ID NO: 222 | 3064 |
| CII898 | CDR | PRT | US20190085084; SEQ ID NO: 194 | 3065 |
| CII899 | CDR | PRT | US20190085084; SEQ ID NO: 194 | 3066 |
| CII900 | CDR | PRT | WO2016164637; SEQ ID NO: 194 | 3067 |
| CII901 | CDR | PRT | US20190085084; SEQ ID NO: 214 | 3068 |
| CII902 | CDR | PRT | US20190085084; SEQ ID NO: 214 | 3069 |
| CII903 | CDR | PRT | WO2016164637; SEQ ID NO: 214 | 3070 |
| CII904 | CDR | PRT | US20190085084; SEQ ID NO: 204 | 3071 |
| CII905 | CDR | PRT | US20190085084; SEQ ID NO: 204 | 3072 |
| CII906 | CDR | PRT | WO2016164637; SEQ ID NO: 204 | 3073 |
| CII907 | CDR | PRT | US20190085084; SEQ ID NO: 203 | 3074 |
| CII908 | CDR | PRT | US20190085084; SEQ ID NO: 203 | 3075 |
| CII909 | CDR | PRT | WO2016164637; SEQ ID NO: 203 | 3076 |
| CII910 | CDR | PRT | US20190085084; SEQ ID NO: 181 | 3077 |
| CII911 | CDR | PRT | US20190085084; SEQ ID NO: 181 | 3078 |
| CII912 | CDR | PRT | WO2016164637; SEQ ID NO: 181 | 3079 |
| CII913 | CDR | PRT | US20190085084; SEQ ID NO: 255 | 3080 |
| CII914 | CDR | PRT | US20190085084; SEQ ID NO: 255 | 3081 |
| CII915 | CDR | PRT | WO2016164637; SEQ ID NO: 255 | 3082 |
| CII916 | CDR | PRT | US20190085084; SEQ ID NO: 180 | 3083 |
| CII917 | CDR | PRT | US20190085084; SEQ ID NO: 180 | 3084 |
| CII918 | CDR | PRT | WO2016164637; SEQ ID NO: 180 | 3085 |
| CII919 | CDR | PRT | US20190085084; SEQ ID NO: 231 | 3086 |
| CII920 | CDR | PRT | US20190085084; SEQ ID NO: 231 | 3087 |
| CII921 | CDR | PRT | WO2016164637; SEQ ID NO: 231 | 3088 |
| CII922 | CDR | PRT | US20190085084; SEQ ID NO: 232 | 3089 |
| CII923 | CDR | PRT | US20190085084; SEQ ID NO: 232 | 3090 |
| CII924 | CDR | PRT | WO2016164637; SEQ ID NO: 232 | 3091 |
| CII925 | CDR | PRT | US20190085084; SEQ ID NO: 256 | 3092 |
| CII926 | CDR | PRT | US20190085084; SEQ ID NO: 256 | 3093 |
| CII927 | CDR | PRT | WO2016164637; SEQ ID NO: 256 | 3094 |
| CII928 | CDR | PRT | US20190085084; SEQ ID NO: 229 | 3095 |
| CII929 | CDR | PRT | US20190085084; SEQ ID NO: 229 | 3096 |
| CII930 | CDR | PRT | WO2016164637; SEQ ID NO: 229 | 3097 |
| CII931 | CDR | PRT | US20190085084; SEQ ID NO: 234 | 3098 |
| CII932 | CDR | PRT | US20190085084; SEQ ID NO: 234 | 3099 |
| CII933 | CDR | PRT | WO2016164637; SEQ ID NO: 234 | 3100 |
| CII934 | CDR | PRT | US20190085084; SEQ ID NO: 254 | 3101 |
| CII935 | CDR | PRT | US20190085084; SEQ ID NO: 254 | 3102 |
| CII936 | CDR | PRT | WO2016164637; SEQ ID NO: 254 | 3103 |
| CII937 | CDR | PRT | US20190085084; SEQ ID NO: 247 | 3104 |
| CII938 | CDR | PRT | US20190085084; SEQ ID NO: 247 | 3105 |
| CII939 | CDR | PRT | WO2016164637; SEQ ID NO: 247 | 3106 |
| CII940 | CDR | PRT | US20190085084; SEQ ID NO: 245 | 3107 |
| CII941 | CDR | PRT | US20190085084; SEQ ID NO: 245 | 3108 |
| CII942 | CDR | PRT | WO2016164637; SEQ ID NO: 245 | 3109 |
| CII943 | CDR | PRT | US20190085084; SEQ ID NO: 253 | 3110 |
| CII944 | CDR | PRT | US20190085084; SEQ ID NO: 253 | 3111 |
| CII945 | CDR | PRT | WO2016164637; SEQ ID NO: 253 | 3112 |
| CII946 | CDR | PRT | US20190085084; SEQ ID NO: 183 | 3113 |
| CII947 | CDR | PRT | US20190085084; SEQ ID NO: 183 | 3114 |
| CII948 | CDR | PRT | WO2016164637; SEQ ID NO: 183 | 3115 |
| CII949 | CDR | PRT | US20190085084; SEQ ID NO: 239 | 3116 |
| CII950 | CDR | PRT | US20190085084; SEQ ID NO: 239 | 3117 |
| CII951 | CDR | PRT | WO2016164637; SEQ ID NO: 239 | 3118 |
| CII952 | CDR | PRT | US20190085084; SEQ ID NO: 243 | 3119 |
| CII953 | CDR | PRT | US20190085084; SEQ ID NO: 243 | 3120 |
| CII954 | CDR | PRT | WO2016164637; SEQ ID NO: 243 | 3121 |
| CII955 | CDR | PRT | US20190085084; SEQ ID NO: 185 | 3122 |
| CII956 | CDR | PRT | US20190085084; SEQ ID NO: 185 | 3123 |
| CII957 | CDR | PRT | WO2016164637; SEQ ID NO: 185 | 3124 |
| CII958 | CDR | PRT | US20190085084; SEQ ID NO: 197 | 3125 |
| CII959 | CDR | PRT | US20190085084; SEQ ID NO: 197 | 3126 |
| CII960 | CDR | PRT | WO2016164637; SEQ ID NO: 197 | 3127 |
| CII961 | CDR | PRT | US20190085084; SEQ ID NO: 233 | 3128 |
| CII962 | CDR | PRT | US20190085084; SEQ ID NO: 233 | 3129 |
| CII963 | CDR | PRT | WO2016164637; SEQ ID NO: 233 | 3130 |
| CII964 | CDR | PRT | US20190085084; SEQ ID NO: 182 | 3131 |
| CII965 | CDR | PRT | US20190085084; SEQ ID NO: 182 | 3132 |
| CII966 | CDR | PRT | WO2016164637; SEQ ID NO: 182 | 3133 |
| CII967 | CDR | PRT | US20190085084; SEQ ID NO: 249 | 3134 |
| CII968 | CDR | PRT | US20190085084; SEQ ID NO: 249 | 3135 |
| CII969 | CDR | PRT | WO2016164637; SEQ ID NO: 249 | 3136 |
| CII970 | CDR | PRT | US20190085084; SEQ ID NO: 179 | 3137 |
| CII971 | CDR | PRT | US20190085084; SEQ ID NO: 179 | 3138 |
| CII972 | CDR | PRT | WO2016164637; SEQ ID NO: 179 | 3139 |
| CII973 | CDR | PRT | US20190085084; SEQ ID NO: 191 | 3140 |
| CII974 | CDR | PRT | US20190085084; SEQ ID NO: 191 | 3141 |
| CII975 | CDR | PRT | WO2016164637; SEQ ID NO: 191 | 3142 |
| CII976 | CDR | PRT | US20190085084; SEQ ID NO: 219 | 3143 |
| CII977 | CDR | PRT | US20190085084; SEQ ID NO: 219 | 3144 |
| CII978 | CDR | PRT | WO2016164637; SEQ ID NO: 219 | 3145 |
| CII979 | CDR | PRT | US20190085084; SEQ ID NO: 33 | 3146 |
| CII980 | CDR | PRT | US20190085084; SEQ ID NO: 33 | 3147 |
| CII981 | CDR | PRT | WO2016164637; SEQ ID NO: 33 | 3148 |
| CII982 | CDR | PRT | US20190085084; SEQ ID NO: 34 | 3149 |
| CII983 | CDR | PRT | US20190085084; SEQ ID NO: 34 | 3150 |
| CII984 | CDR | PRT | WO2016164637; SEQ ID NO: 34 | 3151 |
| CII985 | CDR | PRT | US20190085084; SEQ ID NO: 47 | 3152 |
| CII986 | CDR | PRT | US20190085084; SEQ ID NO: 47 | 3153 |
| CII987 | CDR | PRT | WO2016164637; SEQ ID NO: 47 | 3154 |
| CII988 | CDR | PRT | US20190085084; SEQ ID NO: 39 | 3155 |
| CII989 | CDR | PRT | US20190085084; SEQ ID NO: 39 | 3156 |
| CII990 | CDR | PRT | WO2016164637; SEQ ID NO: 39 | 3157 |
| CII991 | CDR | PRT | US20190085084; SEQ ID NO: 106 | 3158 |
| CII992 | CDR | PRT | US20190085084; SEQ ID NO: 106 | 3159 |
| CII993 | CDR | PRT | WO2016164637; SEQ ID NO: 106 | 3160 |
| CII994 | CDR | PRT | US20190085084; SEQ ID NO: 125 | 3161 |
| CII995 | CDR | PRT | US20190085084; SEQ ID NO: 125 | 3162 |
| CII996 | CDR | PRT | WO2016164637; SEQ ID NO: 125 | 3163 |
| CII997 | CDR | PRT | US20190085084; SEQ ID NO: 97 | 3164 |
| CII998 | CDR | PRT | US20190085084; SEQ ID NO: 97 | 3165 |
| CII999 | CDR | PRT | WO2016164637; SEQ ID NO: 97 | 3166 |
| CII1000 | CDR | PRT | US20190085084; SEQ ID NO: 134 | 3167 |
| CII1001 | CDR | PRT | US20190085084; SEQ ID NO: 134 | 3168 |
| CII1002 | CDR | PRT | WO2016164637; SEQ ID NO: 134 | 3169 |
| CII1003 | CDR | PRT | US20190085084; SEQ ID NO: 133 | 3170 |
| CII1004 | CDR | PRT | US20190085084; SEQ ID NO: 133 | 3171 |
| CII1005 | CDR | PRT | WO2016164637; SEQ ID NO: 133 | 3172 |
| CII1006 | CDR | PRT | US20190085084; SEQ ID NO: 138 | 3173 |
| CII1007 | CDR | PRT | WO2016164637; SEQ ID NO: 138 | 3174 |
| CII1008 | CDR | PRT | US20190085084; SEQ ID NO: 137 | 3175 |
| CII1009 | CDR | PRT | WO2016164637; SEQ ID NO: 137 | 3176 |
| CII1010 | CDR | PRT | US20190085084; SEQ ID NO: 136 | 3177 |
| CII1011 | CDR | PRT | US20190085084; SEQ ID NO: 136 | 3178 |
| CII1012 | CDR | PRT | WO2016164637; SEQ ID NO: 136 | 3179 |
| CII1013 | CDR | PRT | US20190085084; SEQ ID NO: 140 | 3180 |
| CII1014 | CDR | PRT | US20190085084; SEQ ID NO: 140 | 3181 |
| CII1015 | CDR | PRT | WO2016164637; SEQ ID NO: 140 | 3182 |
| CII1016 | CDR | PRT | US20190085084; SEQ ID NO: 135 | 3183 |
| CII1017 | CDR | PRT | US20190085084; SEQ ID NO: 135 | 3184 |
| CII1018 | CDR | PRT | WO2016164637; SEQ ID NO: 135 | 3185 |
| CII1019 | CDR | PRT | US20190085084; SEQ ID NO: 139 | 3186 |
| CII1020 | CDR | PRT | WO2016164637; SEQ ID NO: 139 | 3187 |
| CII1021 | CDR | PRT | US20190085084; SEQ ID NO: 31 | 3188 |
| CII1022 | CDR | PRT | WO2016164637; SEQ ID NO: 31 | 3189 |
| CII1023 | CDR | PRT | US20190085084; SEQ ID NO: 26 | 3190 |
| CII1024 | CDR | PRT | US20190085084; SEQ ID NO: 26 | 3191 |
| CII1025 | CDR | PRT | WO2016164637; SEQ ID NO: 26 | 3192 |
| CII1026 | CDR | PRT | US20190085084; SEQ ID NO: 29 | 3193 |
| CII1027 | CDR | PRT | US20190085084; SEQ ID NO: 29 | 3194 |
| CII1028 | CDR | PRT | WO2016164637; SEQ ID NO: 29 | 3195 |
| CII1029 | CDR | PRT | US20190085084; SEQ ID NO: 574 | 3196 |
| CII1030 | CDR | PRT | US20190085084; SEQ ID NO: 574 | 3197 |
| CII1031 | CDR | PRT | WO2016164637; SEQ ID NO: 574 | 3198 |
| CII1032 | CDR | PRT | US20190085084; SEQ ID NO: 577 | 3199 |
| CII1033 | CDR | PRT | WO2016164637; SEQ ID NO: 577 | 3200 |
| CII1034 | CDR | PRT | US20190085084; SEQ ID NO: 572 | 3201 |
| CII1035 | CDR | PRT | WO2016164637; SEQ ID NO: 572 | 3202 |
| CII1036 | CDR | PRT | US20190085084; SEQ ID NO: 573 | 3203 |
| CII1037 | CDR | PRT | US20190085084; SEQ ID NO: 573 | 3204 |
| CII1038 | CDR | PRT | WO2016164637; SEQ ID NO: 573 | 3205 |
| CII1039 | CDR | PRT | US20190085084; SEQ ID NO: 156 | 3206 |
| CII1040 | CDR | PRT | US20190085084; SEQ ID NO: 156 | 3207 |
| CII1041 | CDR | PRT | WO2016164637; SEQ ID NO: 156 | 3208 |
| CII1042 | CDR | PRT | US20190085084; SEQ ID NO: 158 | 3209 |
| CII1043 | CDR | PRT | US20190085084; SEQ ID NO: 158 | 3210 |
| CII1044 | CDR | PRT | WO2016164637; SEQ ID NO: 158 | 3211 |
| CII1045 | CDR | PRT | US20190085084; SEQ ID NO: 579 | 3212 |
| CII1046 | CDR | PRT | US20190085084; SEQ ID NO: 579 | 3213 |
| CII1047 | CDR | PRT | WO2016164637; SEQ ID NO: 579 | 3214 |
| CII1048 | CDR | PRT | US20190085084; SEQ ID NO: 578 | 3215 |
| CII1049 | CDR | PRT | WO2016164637; SEQ ID NO: 578 | 3216 |
| CII1050 | CDR | PRT | US20190085084; SEQ ID NO: 575 | 3217 |
| CII1051 | CDR | PRT | WO2016164637; SEQ ID NO: 575 | 3218 |
| CII1052 | CDR | PRT | US20190085084; SEQ ID NO: 576 | 3219 |
| CII1053 | CDR | PRT | US20190085084; SEQ ID NO: 576 | 3220 |
| CII1054 | CDR | PRT | WO2016164637; SEQ ID NO: 576 | 3221 |
| CII1055 | CDR | PRT | US20190085084; SEQ ID NO: 502 | 3222 |
| CII1056 | CDR | PRT | US20190085084; SEQ ID NO: 502 | 3223 |
| CII1057 | CDR | PRT | WO2016164637; SEQ ID NO: 502 | 3224 |
| CII1058 | CDR | PRT | US20190085084; SEQ ID NO: 160 | 3225 |
| CII1059 | CDR | PRT | US20190085084; SEQ ID NO: 160 | 3226 |
| CII1060 | CDR | PRT | WO2016164637; SEQ ID NO: 160 | 3227 |
| CII1061 | CDR | PRT | US20190085084; SEQ ID NO: 571 | 3228 |
| CII1062 | CDR | PRT | WO2016164637; SEQ ID NO: 571 | 3229 |
| CII1063 | CDR | PRT | US20190085084; SEQ ID NO: 566 | 3230 |
| CII1064 | CDR | PRT | US20190085084; SEQ ID NO: 566 | 3231 |
| CII1065 | CDR | PRT | WO2016164637; SEQ ID NO: 566 | 3232 |
| CII1066 | CDR | PRT | US20190085084; SEQ ID NO: 597 | 3233 |
| CII1067 | CDR | PRT | US20190085084; SEQ ID NO: 597 | 3234 |
| CII1068 | CDR | PRT | WO2016164637; SEQ ID NO: 597 | 3235 |
| CII1069 | CDR | PRT | US20190085084; SEQ ID NO: 593 | 3236 |
| CII1070 | CDR | PRT | US20190085084; SEQ ID NO: 593 | 3237 |
| CII1071 | CDR | PRT | WO2016164637; SEQ ID NO: 593 | 3238 |
| CII1072 | CDR | PRT | US20190085084; SEQ ID NO: 144 | 3239 |
| CII1073 | CDR | PRT | US20190085084; SEQ ID NO: 144 | 3240 |
| CII1074 | CDR | PRT | WO2016164637; SEQ ID NO: 144 | 3241 |
| CII1075 | CDR | PRT | US20190085084; SEQ ID NO: 145 | 3242 |
| CII1076 | CDR | PRT | US20190085084; SEQ ID NO: 145 | 3243 |
| CII1077 | CDR | PRT | WO2016164637; SEQ ID NO: 145 | 3244 |
| CII1078 | CDR | PRT | US20190085084; SEQ ID NO: 596 | 3245 |
| CII1079 | CDR | PRT | US20190085084; SEQ ID NO: 596 | 3246 |
| CII1080 | CDR | PRT | WO2016164637; SEQ ID NO: 596 | 3247 |
| CII1081 | CDR | PRT | US20190085084; SEQ ID NO: 147 | 3248 |
| CII1082 | CDR | PRT | US20190085084; SEQ ID NO: 147 | 3249 |
| CII1083 | CDR | PRT | WO2016164637; SEQ ID NO: 147 | 3250 |
| CII1084 | CDR | PRT | US20190085084; SEQ ID NO: 595 | 3251 |
| CII1085 | CDR | PRT | US20190085084; SEQ ID NO: 595 | 3252 |
| CII1086 | CDR | PRT | WO2016164637; SEQ ID NO: 595 | 3253 |
| CII1087 | CDR | PRT | US20190085084; SEQ ID NO: 148 | 3254 |
| CII1088 | CDR | PRT | US20190085084; SEQ ID NO: 148 | 3255 |
| CII1089 | CDR | PRT | WO2016164637; SEQ ID NO: 148 | 3256 |
| CII1090 | CDR | PRT | US20190085084; SEQ ID NO: 591 | 3257 |
| CII1091 | CDR | PRT | US20190085084; SEQ ID NO: 591 | 3258 |
| CII1092 | CDR | PRT | WO2016164637; SEQ ID NO: 591 | 3259 |
| CII1093 | CDR | PRT | US20190085084; SEQ ID NO: 592 | 3260 |
| CII1094 | CDR | PRT | US20190085084; SEQ ID NO: 592 | 3261 |
| CII1095 | CDR | PRT | WO2016164637; SEQ ID NO: 592 | 3262 |
| CII1096 | CDR | PRT | US20190085084; SEQ ID NO: 568 | 3263 |
| CII1097 | CDR | PRT | US20190085084; SEQ ID NO: 568 | 3264 |
| CII1098 | CDR | PRT | WO2016164637; SEQ ID NO: 568 | 3265 |
| CII1099 | CDR | PRT | US20190085084; SEQ ID NO: 570 | 3266 |
| CII1100 | CDR | PRT | US20190085084; SEQ ID NO: 570 | 3267 |
| CII1101 | CDR | PRT | WO2016164637; SEQ ID NO: 570 | 3268 |
| CII1102 | CDR | PRT | US20190085084; SEQ ID NO: 567 | 3269 |
| CII1103 | CDR | PRT | US20190085084; SEQ ID NO: 567 | 3270 |
| CII1104 | CDR | PRT | WO2016164637; SEQ ID NO: 567 | 3271 |
| CII1105 | CDR | PRT | US20190085084; SEQ ID NO: 130 | 3272 |
| CII1106 | CDR | PRT | US20190085084; SEQ ID NO: 130 | 3273 |
| CII1107 | CDR | PRT | WO2016164637; SEQ ID NO: 130 | 3274 |
| CII1108 | CDR | PRT | US20190085084; SEQ ID NO: 496 | 3275 |
| CII1109 | CDR | PRT | US20190085084; SEQ ID NO: 496 | 3276 |
| CII1110 | CDR | PRT | WO2016164637; SEQ ID NO: 496 | 3277 |
| CII1111 | CDR | PRT | US20190085084; SEQ ID NO: 535 | 3278 |
| CII1112 | CDR | PRT | US20190085084; SEQ ID NO: 535 | 3279 |
| CII1113 | CDR | PRT | WO2016164637; SEQ ID NO: 535 | 3280 |
| CII1114 | CDR | PRT | US20190085084; SEQ ID NO: 537 | 3281 |
| CII1115 | CDR | PRT | US20190085084; SEQ ID NO: 537 | 3282 |
| CII1116 | CDR | PRT | WO2016164637; SEQ ID NO: 537 | 3283 |
| CII1117 | CDR | PRT | US20190085084; SEQ ID NO: 533 | 3284 |
| CII1118 | CDR | PRT | US20190085084; SEQ ID NO: 533 | 3285 |
| CII1119 | CDR | PRT | WO2016164637; SEQ ID NO: 533 | 3286 |
| CII1120 | CDR | PRT | US20190085084; SEQ ID NO: 534 | 3287 |
| CII1121 | CDR | PRT | US20190085084; SEQ ID NO: 534 | 3288 |
| CII1122 | CDR | PRT | WO2016164637; SEQ ID NO: 534 | 3289 |
| CII1123 | CDR | PRT | US20190085084; SEQ ID NO: 536 | 3290 |
| CII1124 | CDR | PRT | US20190085084; SEQ ID NO: 536 | 3291 |
| CII1125 | CDR | PRT | WO2016164637; SEQ ID NO: 536 | 3292 |
| CII1126 | CDR | PRT | US20190085084; SEQ ID NO: 531 | 3293 |
| CII1127 | CDR | PRT | US20190085084; SEQ ID NO: 531 | 3294 |
| CII1128 | CDR | PRT | WO2016164637; SEQ ID NO: 531 | 3295 |
| CII1129 | CDR | PRT | US20190085084; SEQ ID NO: 155 | 3296 |
| CII1130 | CDR | PRT | US20190085084; SEQ ID NO: 155 | 3297 |
| CII1131 | CDR | PRT | WO2016164637; SEQ ID NO: 155 | 3298 |
| CII1132 | CDR | PRT | US20190085084; SEQ ID NO: 530 | 3299 |
| CII1133 | CDR | PRT | US20190085084; SEQ ID NO: 530 | 3300 |
| CII1134 | CDR | PRT | WO2016164637; SEQ ID NO: 530 | 3301 |
| CII1135 | CDR | PRT | US20190085084; SEQ ID NO: 527 | 3302 |
| CII1136 | CDR | PRT | US20190085084; SEQ ID NO: 527 | 3303 |
| CII1137 | CDR | PRT | WO2016164637; SEQ ID NO: 527 | 3304 |
| CII1138 | CDR | PRT | US20190085084; SEQ ID NO: 529 | 3305 |
| CII1139 | CDR | PRT | US20190085084; SEQ ID NO: 529 | 3306 |
| CII1140 | CDR | PRT | WO2016164637; SEQ ID NO: 529 | 3307 |
| CII1141 | CDR | PRT | US20190085084; SEQ ID NO: 525 | 3308 |
| CII1142 | CDR | PRT | US20190085084; SEQ ID NO: 525 | 3309 |
| CII1143 | CDR | PRT | WO2016164637; SEQ ID NO: 525 | 3310 |
| CII1144 | CDR | PRT | US20190085084; SEQ ID NO: 526 | 3311 |
| CII1145 | CDR | PRT | US20190085084; SEQ ID NO: 526 | 3312 |
| CII1146 | CDR | PRT | WO2016164637; SEQ ID NO: 526 | 3313 |
| CII1147 | CDR | PRT | US20190085084; SEQ ID NO: 539 | 3314 |
| CII1148 | CDR | PRT | US20190085084; SEQ ID NO: 539 | 3315 |
| CII1149 | CDR | PRT | WO2016164637; SEQ ID NO: 539 | 3316 |
| CII1150 | CDR | PRT | US20190085084; SEQ ID NO: 178 | 3317 |
| CII1151 | CDR | PRT | US20190085084; SEQ ID NO: 178 | 3318 |
| CII1152 | CDR | PRT | WO2016164637; SEQ ID NO: 178 | 3319 |
| CII1153 | CDR | PRT | US20190085084; SEQ ID NO: 538 | 3320 |
| CII1154 | CDR | PRT | US20190085084; SEQ ID NO: 538 | 3321 |
| CII1155 | CDR | PRT | WO2016164637; SEQ ID NO: 538 | 3322 |
| CII1156 | CDR | PRT | US20190085084; SEQ ID NO: 28 | 3323 |
| CII1157 | CDR | PRT | US20190085084; SEQ ID NO: 28 | 3324 |
| CII1158 | CDR | PRT | WO2016164637; SEQ ID NO: 28 | 3325 |
| CII1159 | CDR | PRT | US20190085084; SEQ ID NO: 25 | 3326 |
| CII1160 | CDR | PRT | US20190085084; SEQ ID NO: 25 | 3327 |
| CII1161 | CDR | PRT | WO2016164637; SEQ ID NO: 25 | 3328 |
| CII1162 | CDR | PRT | US20190085084; SEQ ID NO: 40 | 3329 |
| CII1163 | CDR | PRT | US20190085084; SEQ ID NO: 40 | 3330 |
| CII1164 | CDR | PRT | WO2016164637; SEQ ID NO: 40 | 3331 |
| CII1165 | CDR | PRT | US20190085084; SEQ ID NO: 38 | 3332 |
| CII1166 | CDR | PRT | US20190085084; SEQ ID NO: 38 | 3333 |
| CII1167 | CDR | PRT | WO2016164637; SEQ ID NO: 38 | 3334 |
| CII1168 | CDR | PRT | US20190085084; SEQ ID NO: 30 | 3335 |
| CII1169 | CDR | PRT | US20190085084; SEQ ID NO: 30 | 3336 |
| CII1170 | CDR | PRT | WO2016164637; SEQ ID NO: 30 | 3337 |
| CII1171 | CDR | PRT | US20190085084; SEQ ID NO: 586 | 3338 |
| CII1172 | CDR | PRT | US20190085084; SEQ ID NO: 586 | 3339 |
| CII1173 | CDR | PRT | WO2016164637; SEQ ID NO: 586 | 3340 |
| CII1174 | CDR | PRT | US20190085084; SEQ ID NO: 581 | 3341 |
| CII1175 | CDR | PRT | US20190085084; SEQ ID NO: 581 | 3342 |
| CII1176 | CDR | PRT | WO2016164637; SEQ ID NO: 581 | 3343 |
| CII1177 | CDR | PRT | US20190085084; SEQ ID NO: 602 | 3344 |
| CII1178 | CDR | PRT | US20190085084; SEQ ID NO: 602 | 3345 |
| CII1179 | CDR | PRT | WO2016164637; SEQ ID NO: 602 | 3346 |
| CII1180 | CDR | PRT | US20190085084; SEQ ID NO: 124 | 3347 |
| CII1181 | CDR | PRT | US20190085084; SEQ ID NO: 124 | 3348 |
| CII1182 | CDR | PRT | WO2016164637; SEQ ID NO: 124 | 3349 |
| CII1183 | CDR | PRT | US20190085084; SEQ ID NO: 79 | 3350 |
| CII1184 | CDR | PRT | US20190085084; SEQ ID NO: 79 | 3351 |
| CII1185 | CDR | PRT | WO2016164637; SEQ ID NO: 79 | 3352 |
| CII1186 | CDR | PRT | US20190085084; SEQ ID NO: 121 | 3353 |
| CII1187 | CDR | PRT | US20190085084; SEQ ID NO: 121 | 3354 |
| CII1188 | CDR | PRT | WO2016164637; SEQ ID NO: 121 | 3355 |
| CII1189 | CDR | PRT | US20190085084; SEQ ID NO: 90 | 3356 |
| CII1190 | CDR | PRT | US20190085084; SEQ ID NO: 90 | 3357 |
| CII1191 | CDR | PRT | WO2016164637; SEQ ID NO: 90 | 3358 |
| CII1192 | CDR | PRT | US20190085084; SEQ ID NO: 70 | 3359 |
| CII1193 | CDR | PRT | US20190085084; SEQ ID NO: 70 | 3360 |
| CII1194 | CDR | PRT | WO2016164637; SEQ ID NO: 70 | 3361 |
| CII1195 | CDR | PRT | US20190085084; SEQ ID NO: 105 | 3362 |
| CII1196 | CDR | PRT | US20190085084; SEQ ID NO: 105 | 3363 |
| CII1197 | CDR | PRT | WO2016164637; SEQ ID NO: 105 | 3364 |
| CII1198 | CDR | PRT | US20190085084; SEQ ID NO: 72 | 3365 |
| CII1199 | CDR | PRT | US20190085084; SEQ ID NO: 72 | 3366 |
| CII1200 | CDR | PRT | WO2016164637; SEQ ID NO: 72 | 3367 |
| CII1201 | CDR | PRT | US20190085084; SEQ ID NO: 100 | 3368 |
| CII1202 | CDR | PRT | US20190085084; SEQ ID NO: 100 | 3369 |
| CII1203 | CDR | PRT | WO2016164637; SEQ ID NO: 100 | 3370 |
| CII1204 | CDR | PRT | US20190085084; SEQ ID NO: 69 | 3371 |
| CII1205 | CDR | PRT | US20190085084; SEQ ID NO: 69 | 3372 |
| CII1206 | CDR | PRT | WO2016164637; SEQ ID NO: 69 | 3373 |
| CII1207 | CDR | PRT | US20190085084; SEQ ID NO: 107 | 3374 |
| CII1208 | CDR | PRT | US20190085084; SEQ ID NO: 107 | 3375 |
| CII1209 | CDR | PRT | WO2016164637; SEQ ID NO: 107 | 3376 |
| CII1210 | CDR | PRT | US20190085084; SEQ ID NO: 68 | 3377 |
| CII1211 | CDR | PRT | US20190085084; SEQ ID NO: 68 | 3378 |
| CII1212 | CDR | PRT | WO2016164637; SEQ ID NO: 68 | 3379 |
| CII1213 | CDR | PRT | US20190085084; SEQ ID NO: 45 | 3380 |
| CII1214 | CDR | PRT | US20190085084; SEQ ID NO: 45 | 3381 |
| CII1215 | CDR | PRT | WO2016164637; SEQ ID NO: 45 | 3382 |
| CII1216 | CDR | PRT | US20190085084; SEQ ID NO: 174 | 3383 |
| CII1217 | CDR | PRT | WO2016164637; SEQ ID NO: 174 | 3384 |
| CII1218 | CDR | PRT | US20190085084; SEQ ID NO: 101 | 3385 |
| CII1219 | CDR | PRT | US20190085084; SEQ ID NO: 101 | 3386 |
| CII1220 | CDR | PRT | WO2016164637; SEQ ID NO: 101 | 3387 |
| CII1221 | CDR | PRT | US20190085084; SEQ ID NO: 598 | 3388 |
| CII1222 | CDR | PRT | US20190085084; SEQ ID NO: 598 | 3389 |
| CII1223 | CDR | PRT | WO2016164637; SEQ ID NO: 598 | 3390 |
| CII1224 | CDR | PRT | US20190085084; SEQ ID NO: 109 | 3391 |
| CII1225 | CDR | PRT | US20190085084; SEQ ID NO: 109 | 3392 |
| CII1226 | CDR | PRT | WO2016164637; SEQ ID NO: 109 | 3393 |
| CII1227 | CDR | PRT | US20190085084; SEQ ID NO: 111 | 3394 |
| CII1228 | CDR | PRT | US20190085084; SEQ ID NO: 111 | 3395 |
| CII1229 | CDR | PRT | WO2016164637; SEQ ID NO: 111 | 3396 |
| CII1230 | CDR | PRT | US20190085084; SEQ ID NO: 62 | 3397 |
| CII1231 | CDR | PRT | US20190085084; SEQ ID NO: 62 | 3398 |
| CII1232 | CDR | PRT | WO2016164637; SEQ ID NO: 62 | 3399 |
| CII1233 | CDR | PRT | US20190085084; SEQ ID NO: 89 | 3400 |
| CII1234 | CDR | PRT | US20190085084; SEQ ID NO: 89 | 3401 |
| CII1235 | CDR | PRT | WO2016164637; SEQ ID NO: 89 | 3402 |
| CII1236 | CDR | PRT | US20190085084; SEQ ID NO: 48 | 3403 |
| CII1237 | CDR | PRT | US20190085084; SEQ ID NO: 48 | 3404 |
| CII1238 | CDR | PRT | WO2016164637; SEQ ID NO: 48 | 3405 |
| CII1239 | CDR | PRT | US20190085084; SEQ ID NO: 82 | 3406 |
| CII1240 | CDR | PRT | US20190085084; SEQ ID NO: 82 | 3407 |
| CII1241 | CDR | PRT | WO2016164637; SEQ ID NO: 82 | 3408 |
| CII1242 | CDR | PRT | US20190085084; SEQ ID NO: 95 | 3409 |
| CII1243 | CDR | PRT | US20190085084; SEQ ID NO: 95 | 3410 |
| CII1244 | CDR | PRT | WO2016164637; SEQ ID NO: 95 | 3411 |
| CII1245 | CDR | PRT | US20190085084; SEQ ID NO: 112 | 3412 |
| CII1246 | CDR | PRT | US20190085084; SEQ ID NO: 112 | 3413 |
| CII1247 | CDR | PRT | WO2016164637; SEQ ID NO: 112 | 3414 |
| CII1248 | CDR | PRT | US20190085084; SEQ ID NO: 55 | 3415 |
| CII1249 | CDR | PRT | US20190085084; SEQ ID NO: 55 | 3416 |
| CII1250 | CDR | PRT | WO2016164637; SEQ ID NO: 55 | 3417 |
| CII1251 | CDR | PRT | US20190085084; SEQ ID NO: 113 | 3418 |
| CII1252 | CDR | PRT | US20190085084; SEQ ID NO: 113 | 3419 |
| CII1253 | CDR | PRT | WO2016164637; SEQ ID NO: 113 | 3420 |
| CII1254 | CDR | PRT | US20190085084; SEQ ID NO: 98 | 3421 |
| CII1255 | CDR | PRT | US20190085084; SEQ ID NO: 98 | 3422 |
| CII1256 | CDR | PRT | WO2016164637; SEQ ID NO: 98 | 3423 |
| CII1257 | CDR | PRT | US20190085084; SEQ ID NO: 114 | 3424 |
| CII1258 | CDR | PRT | US20190085084; SEQ ID NO: 114 | 3425 |
| CII1259 | CDR | PRT | WO2016164637; SEQ ID NO: 114 | 3426 |
| CII1260 | CDR | PRT | US20190085084; SEQ ID NO: 44 | 3427 |
| CII1261 | CDR | PRT | US20190085084; SEQ ID NO: 44 | 3428 |
| CII1262 | CDR | PRT | WO2016164637; SEQ ID NO: 44 | 3429 |
| CII1263 | CDR | PRT | US20190085084; SEQ ID NO: 58 | 3430 |
| CII1264 | CDR | PRT | US20190085084; SEQ ID NO: 58 | 3431 |
| CII1265 | CDR | PRT | WO2016164637; SEQ ID NO: 58 | 3432 |
| CII1266 | CDR | PRT | US20190085084; SEQ ID NO: 66 | 3433 |
| CII1267 | CDR | PRT | US20190085084; SEQ ID NO: 66 | 3434 |
| CII1268 | CDR | PRT | WO2016164637; SEQ ID NO: 66 | 3435 |
| CII1269 | CDR | PRT | US20190085084; SEQ ID NO: 56 | 3436 |
| CII1270 | CDR | PRT | US20190085084; SEQ ID NO: 56 | 3437 |
| CII1271 | CDR | PRT | WO2016164637; SEQ ID NO: 56 | 3438 |
| CII1272 | CDR | PRT | US20190085084; SEQ ID NO: 99 | 3439 |
| CII1273 | CDR | PRT | US20190085084; SEQ ID NO: 99 | 3440 |
| CII1274 | CDR | PRT | WO2016164637; SEQ ID NO: 99 | 3441 |
| CII1275 | CDR | PRT | US20190085084; SEQ ID NO: 54 | 3442 |
| CII1276 | CDR | PRT | US20190085084; SEQ ID NO: 54 | 3443 |
| CII1277 | CDR | PRT | WO2016164637; SEQ ID NO: 54 | 3444 |
| CII1278 | CDR | PRT | US20190085084; SEQ ID NO: 61 | 3445 |
| CII1279 | CDR | PRT | US20190085084; SEQ ID NO: 61 | 3446 |
| CII1280 | CDR | PRT | WO2016164637; SEQ ID NO: 61 | 3447 |
| CII1281 | CDR | PRT | US20190085084; SEQ ID NO: 84 | 3448 |
| CII1282 | CDR | PRT | US20190085084; SEQ ID NO: 84 | 3449 |
| CII1283 | CDR | PRT | WO2016164637; SEQ ID NO: 84 | 3450 |
| CII1284 | CDR | PRT | US20190085084; SEQ ID NO: 104 | 3451 |
| CII1285 | CDR | PRT | US20190085084; SEQ ID NO: 104 | 3452 |
| CII1286 | CDR | PRT | WO2016164637; SEQ ID NO: 104 | 3453 |
| CII1287 | CDR | PRT | US20190085084; SEQ ID NO: 103 | 3454 |
| CII1288 | CDR | PRT | US20190085084; SEQ ID NO: 103 | 3455 |
| CII1289 | CDR | PRT | WO2016164637; SEQ ID NO: 103 | 3456 |
| CII1290 | CDR | PRT | US20190085084; SEQ ID NO: 83 | 3457 |
| CII1291 | CDR | PRT | US20190085084; SEQ ID NO: 83 | 3458 |
| CII1292 | CDR | PRT | WO2016164637; SEQ ID NO: 83 | 3459 |
| CII1293 | CDR | PRT | US20190085084; SEQ ID NO: 80 | 3460 |
| CII1294 | CDR | PRT | US20190085084; SEQ ID NO: 80 | 3461 |
| CII1295 | CDR | PRT | WO2016164637; SEQ ID NO: 80 | 3462 |
| CII1296 | CDR | PRT | US20190085084; SEQ ID NO: 91 | 3463 |
| CII1297 | CDR | PRT | US20190085084; SEQ ID NO: 91 | 3464 |
| CII1298 | CDR | PRT | WO2016164637; SEQ ID NO: 91 | 3465 |
| CII1299 | CDR | PRT | US20190085084; SEQ ID NO: 42 | 3466 |
| CII1300 | CDR | PRT | US20190085084; SEQ ID NO: 42 | 3467 |
| CII1301 | CDR | PRT | WO2016164637; SEQ ID NO: 42 | 3468 |
| CII1302 | CDR | PRT | US20190085084; SEQ ID NO: 85 | 3469 |
| CII1303 | CDR | PRT | US20190085084; SEQ ID NO: 85 | 3470 |
| CII1304 | CDR | PRT | WO2016164637; SEQ ID NO: 85 | 3471 |
| CII1305 | CDR | PRT | US20190085084; SEQ ID NO: 122 | 3472 |
| CII1306 | CDR | PRT | US20190085084; SEQ ID NO: 122 | 3473 |
| CII1307 | CDR | PRT | WO2016164637; SEQ ID NO: 122 | 3474 |
| CII1308 | CDR | PRT | US20190085084; SEQ ID NO: 41 | 3475 |
| CII1309 | CDR | PRT | US20190085084; SEQ ID NO: 41 | 3476 |
| CII1310 | CDR | PRT | WO2016164637; SEQ ID NO: 41 | 3477 |
| CII1311 | CDR | PRT | US20190085084; SEQ ID NO: 116 | 3478 |
| CII1312 | CDR | PRT | US20190085084; SEQ ID NO: 116 | 3479 |
| CII1313 | CDR | PRT | WO2016164637; SEQ ID NO: 116 | 3480 |
| CII1314 | CDR | PRT | US20190085084; SEQ ID NO: 59 | 3481 |
| CII1315 | CDR | PRT | US20190085084; SEQ ID NO: 59 | 3482 |
| CII1316 | CDR | PRT | WO2016164637; SEQ ID NO: 59 | 3483 |
| CII1317 | CDR | PRT | US20190085084; SEQ ID NO: 46 | 3484 |
| CII1318 | CDR | PRT | US20190085084; SEQ ID NO: 46 | 3485 |
| CII1319 | CDR | PRT | WO2016164637; SEQ ID NO: 46 | 3486 |
| CII1320 | CDR | PRT | US20190085084; SEQ ID NO: 64 | 3487 |
| CII1321 | CDR | PRT | US20190085084; SEQ ID NO: 64 | 3488 |
| CII1322 | CDR | PRT | WO2016164637; SEQ ID NO: 64 | 3489 |
| CII1323 | CDR | PRT | US20190085084; SEQ ID NO: 50 | 3490 |
| CII1324 | CDR | PRT | US20190085084; SEQ ID NO: 50 | 3491 |
| CII1325 | CDR | PRT | WO2016164637; SEQ ID NO: 50 | 3492 |
| CII1326 | CDR | PRT | US20190085084; SEQ ID NO: 118 | 3493 |
| CII1327 | CDR | PRT | US20190085084; SEQ ID NO: 118 | 3494 |
| CII1328 | CDR | PRT | WO2016164637; SEQ ID NO: 118 | 3495 |
| CII1329 | CDR | PRT | US20190085084; SEQ ID NO: 60 | 3496 |
| CII1330 | CDR | PRT | US20190085084; SEQ ID NO: 60 | 3497 |
| CII1331 | CDR | PRT | WO2016164637; SEQ ID NO: 60 | 3498 |
| CII1332 | CDR | PRT | US20190085084; SEQ ID NO: 65 | 3499 |
| CII1333 | CDR | PRT | US20190085084; SEQ ID NO: 65 | 3500 |
| CII1334 | CDR | PRT | WO2016164637; SEQ ID NO: 65 | 3501 |
| CII1335 | CDR | PRT | US20190085084; SEQ ID NO: 74 | 3502 |
| CII1336 | CDR | PRT | US20190085084; SEQ ID NO: 74 | 3503 |
| CII1337 | CDR | PRT | WO2016164637; SEQ ID NO: 74 | 3504 |
| CII1338 | CDR | PRT | US20190085084; SEQ ID NO: 43 | 3505 |
| CII1339 | CDR | PRT | US20190085084; SEQ ID NO: 43 | 3506 |
| CII1340 | CDR | PRT | WO2016164637; SEQ ID NO: 43 | 3507 |
| CII1341 | CDR | PRT | US20190085084; SEQ ID NO: 123 | 3508 |
| CII1342 | CDR | PRT | US20190085084; SEQ ID NO: 123 | 3509 |
| CII1343 | CDR | PRT | WO2016164637; SEQ ID NO: 123 | 3510 |
| CII1344 | CDR | PRT | US20190085084; SEQ ID NO: 115 | 3511 |
| CII1345 | CDR | PRT | US20190085084; SEQ ID NO: 115 | 3512 |
| CII1346 | CDR | PRT | WO2016164637; SEQ ID NO: 115 | 3513 |
| CII1347 | CDR | PRT | US20190085084; SEQ ID NO: 49 | 3514 |
| CII1348 | CDR | PRT | US20190085084; SEQ ID NO: 49 | 3515 |
| CII1349 | CDR | PRT | WO2016164637; SEQ ID NO: 49 | 3516 |
| CII1350 | CDR | PRT | US20190085084; SEQ ID NO: 52 | 3517 |
| CII1351 | CDR | PRT | US20190085084; SEQ ID NO: 52 | 3518 |
| CII1352 | CDR | PRT | WO2016164637; SEQ ID NO: 52 | 3519 |
| CII1353 | CDR | PRT | US20190085084; SEQ ID NO: 88 | 3520 |
| CII1354 | CDR | PRT | US20190085084; SEQ ID NO: 88 | 3521 |
| CII1355 | CDR | PRT | WO2016164637; SEQ ID NO: 88 | 3522 |
| CII1356 | CDR | PRT | US20190085084; SEQ ID NO: 22 | 3523 |
| CII1357 | CDR | PRT | US20190085084; SEQ ID NO: 22 | 3524 |
| CII1358 | CDR | PRT | WO2016164637; SEQ ID NO: 22 | 3525 |
| CII1359 | CDR | PRT | US20190085084; SEQ ID NO: 16 | 3526 |
| CII1360 | CDR | PRT | US20190085084; SEQ ID NO: 16 | 3527 |
| CII1361 | CDR | PRT | WO2016164637; SEQ ID NO: 16 | 3528 |
| CII1362 | CDR | PRT | US20190085084; SEQ ID NO: 21 | 3529 |
| CII1363 | CDR | PRT | WO2016164637; SEQ ID NO: 21 | 3530 |
| CII1364 | CDR | PRT | US20190085084; SEQ ID NO: 12 | 3531 |
| CII1365 | CDR | PRT | US20190085084; SEQ ID NO: 12 | 3532 |
| CII1366 | CDR | PRT | WO2016164637; SEQ ID NO: 12 | 3533 |
| CII1367 | CDR | PRT | US20190085084; SEQ ID NO: 15 | 3534 |
| CII1368 | CDR | PRT | US20190085084; SEQ ID NO: 15 | 3535 |
| CII1369 | CDR | PRT | WO2016164637; SEQ ID NO: 15 | 3536 |
| CII1370 | CDR | PRT | US20190085084; SEQ ID NO: 24 | 3537 |
| CII1371 | CDR | PRT | US20190085084; SEQ ID NO: 24 | 3538 |
| CII1372 | CDR | PRT | WO2016164637; SEQ ID NO: 24 | 3539 |
| CII1373 | CDR | PRT | US20190085084; SEQ ID NO: 8 | 3540 |
| CII1374 | CDR | PRT | US20190085084; SEQ ID NO: 8 | 3541 |
| CII1375 | CDR | PRT | WO2016164637; SEQ ID NO: 8 | 3542 |
| CII1376 | CDR | PRT | US20190085084; SEQ ID NO: 7 | 3543 |
| CII1377 | CDR | PRT | US20190085084; SEQ ID NO: 7 | 3544 |
| CII1378 | CDR | PRT | WO2016164637; SEQ ID NO: 7 | 3545 |
| CII1379 | CDR | PRT | US20190085084; SEQ ID NO: 512 | 3546 |
| CII1380 | CDR | PRT | US20190085084; SEQ ID NO: 512 | 3547 |
| CII1381 | CDR | PRT | WO2016164637; SEQ ID NO: 512 | 3548 |
| CII1382 | CDR | PRT | US20190085084; SEQ ID NO: 6 | 3549 |
| CII1383 | CDR | PRT | US20190085084; SEQ ID NO: 6 | 3550 |
| CII1384 | CDR | PRT | WO2016164637; SEQ ID NO: 6 | 3551 |
| CII1385 | CDR | PRT | US20190085084; SEQ ID NO: 9 | 3552 |
| CII1386 | CDR | PRT | US20190085084; SEQ ID NO: 9 | 3553 |
| CII1387 | CDR | PRT | WO2016164637; SEQ ID NO: 9 | 3554 |
| CII1388 | CDR | PRT | US20190085084; SEQ ID NO: 11 | 3555 |
| CII1389 | CDR | PRT | US20190085084; SEQ ID NO: 11 | 3556 |
| CII1390 | CDR | PRT | WO2016164637; SEQ ID NO: 11 | 3557 |
| CII1391 | CDR | PRT | US20190085084; SEQ ID NO: 20 | 3558 |
| CII1392 | CDR | PRT | US20190085084; SEQ ID NO: 20 | 3559 |
| CII1393 | CDR | PRT | WO2016164637; SEQ ID NO: 20 | 3560 |
| CII1394 | CDR | PRT | US20190085084; SEQ ID NO: 14 | 3561 |
| CII1395 | CDR | PRT | US20190085084; SEQ ID NO: 14 | 3562 |
| CII1396 | CDR | PRT | WO2016164637; SEQ ID NO: 14 | 3563 |
| CII1397 | CDR | PRT | US20190085084; SEQ ID NO: 13 | 3564 |
| CII1398 | CDR | PRT | US20190085084; SEQ ID NO: 13 | 3565 |
| CII1399 | CDR | PRT | WO2016164637; SEQ ID NO: 13 | 3566 |
| CII1400 | CDR | PRT | US20190085084; SEQ ID NO: 508 | 3567 |
| CII1401 | CDR | PRT | US20190085084; SEQ ID NO: 508 | 3568 |
| CII1402 | CDR | PRT | WO2016164637; SEQ ID NO: 508 | 3569 |
| CII1403 | CDR | PRT | US20190085084; SEQ ID NO: 585 | 3570 |
| CII1404 | CDR | PRT | US20190085084; SEQ ID NO: 585 | 3571 |
| CII1405 | CDR | PRT | WO2016164637; SEQ ID NO: 585 | 3572 |
| CII1406 | CDR | PRT | US20190085084; SEQ ID NO: 10 | 3573 |
| CII1407 | CDR | PRT | US20190085084; SEQ ID NO: 10 | 3574 |
| CII1408 | CDR | PRT | WO2016164637; SEQ ID NO: 10 | 3575 |
| CII1409 | CDR | PRT | US20190085084; SEQ ID NO: 584 | 3576 |
| CII1410 | CDR | PRT | US20190085084; SEQ ID NO: 584 | 3577 |
| CII1411 | CDR | PRT | WO2016164637; SEQ ID NO: 584 | 3578 |
| CII1412 | CDR | PRT | US20190085084; SEQ ID NO: 580 | 3579 |
| CII1413 | CDR | PRT | US20190085084; SEQ ID NO: 580 | 3580 |
| CII1414 | CDR | PRT | WO2016164637; SEQ ID NO: 580 | 3581 |
| CII1415 | CDR | PRT | US20190085084; SEQ ID NO: 35 | 3582 |
| CII1416 | CDR | PRT | US20190085084; SEQ ID NO: 35 | 3583 |
| CII1417 | CDR | PRT | WO2016164637; SEQ ID NO: 35 | 3584 |
| CII1418 | CDR | PRT | US20190085084; SEQ ID NO: 37 | 3585 |
| CII1419 | CDR | PRT | US20190085084; SEQ ID NO: 37 | 3586 |
| CII1420 | CDR | PRT | WO2016164637; SEQ ID NO: 37 | 3587 |
| CII1421 | CDR | PRT | US20190085084; SEQ ID NO: 157 | 3588 |
| CII1422 | CDR | PRT | US20190085084; SEQ ID NO: 157 | 3589 |
| CII1423 | CDR | PRT | WO2016164637; SEQ ID NO: 157 | 3590 |
| CII1424 | CDR | PRT | US20190085084; SEQ ID NO: 151 | 3591 |
| CII1425 | CDR | PRT | US20190085084; SEQ ID NO: 151 | 3592 |
| CII1426 | CDR | PRT | WO2016164637; SEQ ID NO: 151 | 3593 |
| CII1427 | CDR | PRT | US20190085084; SEQ ID NO: 152 | 3594 |
| CII1428 | CDR | PRT | US20190085084; SEQ ID NO: 152 | 3595 |
| CII1429 | CDR | PRT | WO2016164637; SEQ ID NO: 152 | 3596 |
| CII1430 | CDR | PRT | US20190085084; SEQ ID NO: 165 | 3597 |
| CII1431 | CDR | PRT | US20190085084; SEQ ID NO: 165 | 3598 |
| CII1432 | CDR | PRT | WO2016164637; SEQ ID NO: 165 | 3599 |
| CII1433 | CDR | PRT | US20190085084; SEQ ID NO: 162 | 3600 |
| CII1434 | CDR | PRT | US20190085084; SEQ ID NO: 162 | 3601 |
| CII1435 | CDR | PRT | WO2016164637; SEQ ID NO: 162 | 3602 |
| CII1436 | CDR | PRT | US20190085084; SEQ ID NO: 161 | 3603 |
| CII1437 | CDR | PRT | US20190085084; SEQ ID NO: 161 | 3604 |
| CII1438 | CDR | PRT | WO2016164637; SEQ ID NO: 161 | 3605 |
| CII1439 | CDR | PRT | US20190085084; SEQ ID NO: 177 | 3606 |
| CII1440 | CDR | PRT | US20190085084; SEQ ID NO: 177 | 3607 |
| CII1441 | CDR | PRT | WO2016164637; SEQ ID NO: 177 | 3608 |
| CII1442 | CDR | PRT | US20190085084; SEQ ID NO: 171 | 3609 |
| CII1443 | CDR | PRT | US20190085084; SEQ ID NO: 171 | 3610 |
| CII1444 | CDR | PRT | WO2016164637; SEQ ID NO: 171 | 3611 |
| CII1445 | CDR | PRT | US20190085084; SEQ ID NO: 169 | 3612 |
| CII1446 | CDR | PRT | US20190085084; SEQ ID NO: 169 | 3613 |
| CII1447 | CDR | PRT | WO2016164637; SEQ ID NO: 169 | 3614 |
| CII1448 | CDR | PRT | US20190085084; SEQ ID NO: 600 | 3615 |
| CII1449 | CDR | PRT | US20190085084; SEQ ID NO: 600 | 3616 |
| CII1450 | CDR | PRT | WO2016164637; SEQ ID NO: 600 | 3617 |
| CII1451 | CDR | PRT | US20190085084; SEQ ID NO: 603 | 3618 |
| CII1452 | CDR | PRT | US20190085084; SEQ ID NO: 603 | 3619 |
| CII1453 | CDR | PRT | WO2016164637; SEQ ID NO: 603 | 3620 |
| CII1454 | CDR | PRT | US20190085084; SEQ ID NO: 599 | 3621 |
| CII1455 | CDR | PRT | US20190085084; SEQ ID NO: 599 | 3622 |
| CII1456 | CDR | PRT | WO2016164637; SEQ ID NO: 599 | 3623 |
| CII1457 | CDR | PRT | US20190085084; SEQ ID NO: 601 | 3624 |
| CII1458 | CDR | PRT | US20190085084; SEQ ID NO: 601 | 3625 |
| CII1459 | CDR | PRT | WO2016164637; SEQ ID NO: 601 | 3626 |
| CII1460 | CDR | PRT | US20190085084; SEQ ID NO: 173 | 3627 |
| CII1461 | CDR | PRT | US20190085084; SEQ ID NO: 173 | 3628 |
| CII1462 | CDR | PRT | WO2016164637; SEQ ID NO: 173 | 3629 |
| CII1463 | CDR | PRT | US20190085084; SEQ ID NO: 170 | 3630 |
| CII1464 | CDR | PRT | US20190085084; SEQ ID NO: 170 | 3631 |
| CII1465 | CDR | PRT | WO2016164637; SEQ ID NO: 170 | 3632 |
| CII1466 | CDR | PRT | US20190085084; SEQ ID NO: 551 | 3633 |
| CII1467 | CDR | PRT | US20190085084; SEQ ID NO: 551 | 3634 |
| CII1468 | CDR | PRT | WO2016164637; SEQ ID NO: 551 | 3635 |
| CII1469 | CDR | PRT | US20190085084; SEQ ID NO: 528 | 3636 |
| CII1470 | CDR | PRT | WO2016164637; SEQ ID NO: 528 | 3637 |
| CII1471 | CDR | PRT | US20190085084; SEQ ID NO: 532 | 3638 |
| CII1472 | CDR | PRT | US20190085084; SEQ ID NO: 532 | 3639 |
| CII1473 | CDR | PRT | WO2016164637; SEQ ID NO: 532 | 3640 |
| CII1474 | CDR | PRT | US20190085084; SEQ ID NO: 166 | 3641 |
| CII1475 | CDR | PRT | WO2016164637; SEQ ID NO: 166 | 3642 |
| CII1476 | CDR | PRT | US20190085084; SEQ ID NO: 550 | 3643 |
| CII1477 | CDR | PRT | WO2016164637; SEQ ID NO: 550 | 3644 |
| CII1478 | CDR | PRT | US20190085084; SEQ ID NO: 36 | 3645 |
| CII1479 | CDR | PRT | US20190085084; SEQ ID NO: 36 | 3646 |
| CII1480 | CDR | PRT | WO2016164637; SEQ ID NO: 36 | 3647 |
| CII1481 | CDR | PRT | US20190085084; SEQ ID NO: 506 | 3648 |
| CII1482 | CDR | PRT | US20190085084; SEQ ID NO: 506 | 3649 |
| CII1483 | CDR | PRT | WO2016164637; SEQ ID NO: 506 | 3650 |
| CII1484 | CDR | PRT | US20190085084; SEQ ID NO: 494 | 3651 |
| CII1485 | CDR | PRT | US20190085084; SEQ ID NO: 494 | 3652 |
| CII1486 | CDR | PRT | WO2016164637; SEQ ID NO: 494 | 3653 |
| CII1487 | CDR | PRT | US20190085084; SEQ ID NO: 493 | 3654 |
| CII1488 | CDR | PRT | US20190085084; SEQ ID NO: 493 | 3655 |
| CII1489 | CDR | PRT | WO2016164637; SEQ ID NO: 493 | 3656 |
| CII1490 | CDR | PRT | US20190085084; SEQ ID NO: 150 | 3657 |
| CII1491 | CDR | PRT | US20190085084; SEQ ID NO: 150 | 3658 |
| CII1492 | CDR | PRT | WO2016164637; SEQ ID NO: 150 | 3659 |
| CII1493 | CDR | PRT | US20190085084; SEQ ID NO: 499 | 3660 |
| CII1494 | CDR | PRT | US20190085084; SEQ ID NO: 499 | 3661 |
| CII1495 | CDR | PRT | WO2016164637; SEQ ID NO: 499 | 3662 |
| CII1496 | CDR | PRT | US20190085084; SEQ ID NO: 500 | 3663 |
| CII1497 | CDR | PRT | US20190085084; SEQ ID NO: 500 | 3664 |
| CII1498 | CDR | PRT | WO2016164637; SEQ ID NO: 500 | 3665 |
| CII1499 | CDR | PRT | US20190085084; SEQ ID NO: 501 | 3666 |
| CII1500 | CDR | PRT | US20190085084; SEQ ID NO: 501 | 3667 |
| CII1501 | CDR | PRT | WO2016164637; SEQ ID NO: 501 | 3668 |
| CII1502 | CDR | PRT | US20190085084; SEQ ID NO: 507 | 3669 |
| CII1503 | CDR | PRT | US20190085084; SEQ ID NO: 507 | 3670 |
| CII1504 | CDR | PRT | WO2016164637; SEQ ID NO: 507 | 3671 |
| CII1505 | CDR | PRT | US20190085084; SEQ ID NO: 503 | 3672 |
| CII1506 | CDR | PRT | US20190085084; SEQ ID NO: 503 | 3673 |
| CII1507 | CDR | PRT | WO2016164637; SEQ ID NO: 503 | 3674 |
| CII1508 | CDR | PRT | US20190085084; SEQ ID NO: 497 | 3675 |
| CII1509 | CDR | PRT | US20190085084; SEQ ID NO: 497 | 3676 |
| CII1510 | CDR | PRT | WO2016164637; SEQ ID NO: 497 | 3677 |
| CII1511 | CDR | PRT | US20190085084; SEQ ID NO: 153 | 3678 |
| CII1512 | CDR | PRT | US20190085084; SEQ ID NO: 153 | 3679 |
| CII1513 | CDR | PRT | WO2016164637; SEQ ID NO: 153 | 3680 |
| CII1514 | CDR | PRT | US20190085084; SEQ ID NO: 505 | 3681 |
| CII1515 | CDR | PRT | US20190085084; SEQ ID NO: 505 | 3682 |
| CII1516 | CDR | PRT | WO2016164637; SEQ ID NO: 505 | 3683 |
| CII1517 | CDR | PRT | US20190085084; SEQ ID NO: 504 | 3684 |
| CII1518 | CDR | PRT | US20190085084; SEQ ID NO: 504 | 3685 |
| CII1519 | CDR | PRT | WO2016164637; SEQ ID NO: 504 | 3686 |
| CII1520 | CDR | PRT | US20190085084; SEQ ID NO: 498 | 3687 |
| CII1521 | CDR | PRT | US20190085084; SEQ ID NO: 498 | 3688 |
| CII1522 | CDR | PRT | WO2016164637; SEQ ID NO: 498 | 3689 |
| CII1523 | CDR | PRT | US20190085084; SEQ ID NO: 495 | 3690 |
| CII1524 | CDR | PRT | US20190085084; SEQ ID NO: 495 | 3691 |
| CII1525 | CDR | PRT | WO2016164637; SEQ ID NO: 495 | 3692 |
| CII1526 | CDR | PRT | US20190085084; SEQ ID NO: 510 | 3693 |
| CII1527 | CDR | PRT | US20190085084; SEQ ID NO: 510 | 3694 |
| CII1528 | CDR | PRT | WO2016164637; SEQ ID NO: 510 | 3695 |
| CII1529 | CDR | PRT | US20190085084; SEQ ID NO: 583 | 3696 |
| CII1530 | CDR | PRT | WO2016164637; SEQ ID NO: 583 | 3697 |
| CII1531 | CDR | PRT | US20190085084; SEQ ID NO: 590 | 3698 |
| CII1532 | CDR | PRT | US20190085084; SEQ ID NO: 590 | 3699 |
| CII1533 | CDR | PRT | WO2016164637; SEQ ID NO: 590 | 3700 |
| CII1534 | CDR | PRT | US20190085084; SEQ ID NO: 511 | 3701 |
| CII1535 | CDR | PRT | US20190085084; SEQ ID NO: 511 | 3702 |
| CII1536 | CDR | PRT | WO2016164637; SEQ ID NO: 511 | 3703 |
| CII1537 | CDR | PRT | US20190085084; SEQ ID NO: 589 | 3704 |
| CII1538 | CDR | PRT | US20190085084; SEQ ID NO: 589 | 3705 |
| CII1539 | CDR | PRT | WO2016164637; SEQ ID NO: 589 | 3706 |
| CII1540 | CDR | PRT | US20190085084; SEQ ID NO: 565 | 3707 |
| CII1541 | CDR | PRT | US20190085084; SEQ ID NO: 565 | 3708 |
| CII1542 | CDR | PRT | WO2016164637; SEQ ID NO: 565 | 3709 |
| CII1543 | CDR | PRT | US20190085084; SEQ ID NO: 481 | 3710 |
| CII1544 | CDR | PRT | US20190085084; SEQ ID NO: 481 | 3711 |
| CII1545 | CDR | PRT | WO2016164637; SEQ ID NO: 481 | 3712 |
| CII1546 | CDR | PRT | US20190085084; SEQ ID NO: 172 | 3713 |
| CII1547 | CDR | PRT | US20190085084; SEQ ID NO: 172 | 3714 |
| CII1548 | CDR | PRT | WO2016164637; SEQ ID NO: 172 | 3715 |
| CII1549 | CDR | PRT | US20190085084; SEQ ID NO: 175 | 3716 |
| CII1550 | CDR | PRT | US20190085084; SEQ ID NO: 175 | 3717 |
| CII1551 | CDR | PRT | WO2016164637; SEQ ID NO: 175 | 3718 |
| CII1552 | CDR | PRT | US20190085084; SEQ ID NO: 149 | 3719 |
| CII1553 | CDR | PRT | US20190085084; SEQ ID NO: 149 | 3720 |
| CII1554 | CDR | PRT | WO2016164637; SEQ ID NO: 149 | 3721 |
| CII1555 | CDR | PRT | US20190085084; SEQ ID NO: 594 | 3722 |
| CII1556 | CDR | PRT | US20190085084; SEQ ID NO: 594 | 3723 |
| CII1557 | CDR | PRT | WO2016164637; SEQ ID NO: 594 | 3724 |
| CII1558 | CDR | PRT | US20190085084; SEQ ID NO: 142 | 3725 |
| CII1559 | CDR | PRT | US20190085084; SEQ ID NO: 142 | 3726 |
| CII1560 | CDR | PRT | WO2016164637; SEQ ID NO: 142 | 3727 |
| CII1561 | CDR | PRT | US20190085084; SEQ ID NO: 587 | 3728 |
| CII1562 | CDR | PRT | WO2016164637; SEQ ID NO: 587 | 3729 |
| CII1563 | CDR | PRT | US20190085084; SEQ ID NO: 582 | 3730 |
| CII1564 | CDR | PRT | WO2016164637; SEQ ID NO: 582 | 3731 |
| CII1565 | CDR | PRT | US20190085084; SEQ ID NO: 517 | 3732 |
| CII1566 | CDR | PRT | US20190085084; SEQ ID NO: 517 | 3733 |
| CII1567 | CDR | PRT | WO2016164637; SEQ ID NO: 517 | 3734 |
| CII1568 | CDR | PRT | US20190085084; SEQ ID NO: 489 | 3735 |
| CII1569 | CDR | PRT | US20190085084; SEQ ID NO: 489 | 3736 |
| CII1570 | CDR | PRT | WO2016164637; SEQ ID NO: 489 | 3737 |
| CII1571 | CDR | PRT | US20190085084; SEQ ID NO: 487 | 3738 |
| CII1572 | CDR | PRT | WO2016164637; SEQ ID NO: 487 | 3739 |
| CII1573 | CDR | PRT | US20190085084; SEQ ID NO: 482 | 3740 |
| CII1574 | CDR | PRT | US20190085084; SEQ ID NO: 482 | 3741 |
| CII1575 | CDR | PRT | WO2016164637; SEQ ID NO: 482 | 3742 |
| CII1576 | CDR | PRT | US20190085084; SEQ ID NO: 491 | 3743 |
| CII1577 | CDR | PRT | US20190085084; SEQ ID NO: 491 | 3744 |
| CII1578 | CDR | PRT | WO2016164637; SEQ ID NO: 491 | 3745 |
| CII1579 | CDR | PRT | US20190085084; SEQ ID NO: 486 | 3746 |
| CII1580 | CDR | PRT | US20190085084; SEQ ID NO: 486 | 3747 |
| CII1581 | CDR | PRT | WO2016164637; SEQ ID NO: 486 | 3748 |
| CII1582 | CDR | PRT | US20190085084; SEQ ID NO: 485 | 3749 |
| CII1583 | CDR | PRT | US20190085084; SEQ ID NO: 485 | 3750 |
| CII1584 | CDR | PRT | WO2016164637; SEQ ID NO: 485 | 3751 |
| CII1585 | CDR | PRT | US20190085084; SEQ ID NO: 492 | 3752 |
| CII1586 | CDR | PRT | US20190085084; SEQ ID NO: 492 | 3753 |
| CII1587 | CDR | PRT | WO2016164637; SEQ ID NO: 492 | 3754 |
| CII1588 | CDR | PRT | US20190085084; SEQ ID NO: 488 | 3755 |
| CII1589 | CDR | PRT | US20190085084; SEQ ID NO: 488 | 3756 |
| CII1590 | CDR | PRT | WO2016164637; SEQ ID NO: 488 | 3757 |
| CII1591 | CDR | PRT | US20190085084; SEQ ID NO: 490 | 3758 |
| CII1592 | CDR | PRT | US20190085084; SEQ ID NO: 490 | 3759 |
| CII1593 | CDR | PRT | WO2016164637; SEQ ID NO: 490 | 3760 |
| CII1594 | CDR | PRT | US20190085084; SEQ ID NO: 569 | 3761 |
| CII1595 | CDR | PRT | US20190085084; SEQ ID NO: 569 | 3762 |
| CII1596 | CDR | PRT | WO2016164637; SEQ ID NO: 569 | 3763 |
| CII1597 | CDR | PRT | US20190085084; SEQ ID NO: 126 | 3764 |
| CII1598 | CDR | PRT | US20190085084; SEQ ID NO: 126 | 3765 |
| CII1599 | CDR | PRT | WO2016164637; SEQ ID NO: 126 | 3766 |
| CII1600 | CDR | PRT | US20190085084; SEQ ID NO: 520 | 3767 |
| CII1601 | CDR | PRT | US20190085084; SEQ ID NO: 520 | 3768 |
| CII1602 | CDR | PRT | WO2016164637; SEQ ID NO: 520 | 3769 |
| CII1603 | CDR | PRT | US20190085084; SEQ ID NO: 522 | 3770 |
| CII1604 | CDR | PRT | US20190085084; SEQ ID NO: 522 | 3771 |
| CII1605 | CDR | PRT | WO2016164637; SEQ ID NO: 522 | 3772 |
| CII1606 | CDR | PRT | US20190085084; SEQ ID NO: 524 | 3773 |
| CII1607 | CDR | PRT | US20190085084; SEQ ID NO: 524 | 3774 |
| CII1608 | CDR | PRT | WO2016164637; SEQ ID NO: 524 | 3775 |
| CII1609 | CDR | PRT | US20190085084; SEQ ID NO: 519 | 3776 |
| CII1610 | CDR | PRT | US20190085084; SEQ ID NO: 519 | 3777 |
| CII1611 | CDR | PRT | WO2016164637; SEQ ID NO: 519 | 3778 |
| CII1612 | CDR | PRT | US20190085084; SEQ ID NO: 521 | 3779 |
| CII1613 | CDR | PRT | US20190085084; SEQ ID NO: 521 | 3780 |
| CII1614 | CDR | PRT | WO2016164637; SEQ ID NO: 521 | 3781 |
| CII1615 | CDR | PRT | US20190085084; SEQ ID NO: 523 | 3782 |
| CII1616 | CDR | PRT | US20190085084; SEQ ID NO: 523 | 3783 |
| CII1617 | CDR | PRT | WO2016164637; SEQ ID NO: 523 | 3784 |
| CII1618 | CDR | PRT | US20190085084; SEQ ID NO: 516 | 3785 |
| CII1619 | CDR | PRT | US20190085084; SEQ ID NO: 516 | 3786 |
| CII1620 | CDR | PRT | WO2016164637; SEQ ID NO: 516 | 3787 |
| CII1621 | CDR | PRT | US20190085084; SEQ ID NO: 513 | 3788 |
| CII1622 | CDR | PRT | US20190085084; SEQ ID NO: 513 | 3789 |
| CII1623 | CDR | PRT | WO2016164637; SEQ ID NO: 513 | 3790 |
| CII1624 | CDR | PRT | US20190085084; SEQ ID NO: 127 | 3791 |
| CII1625 | CDR | PRT | US20190085084; SEQ ID NO: 127 | 3792 |
| CII1626 | CDR | PRT | WO2016164637; SEQ ID NO: 127 | 3793 |
| CII1627 | CDR | PRT | US20190085084; SEQ ID NO: 515 | 3794 |
| CII1628 | CDR | PRT | US20190085084; SEQ ID NO: 515 | 3795 |
| CII1629 | CDR | PRT | WO2016164637; SEQ ID NO: 515 | 3796 |
| CII1630 | CDR | PRT | US20190085084; SEQ ID NO: 484 | 3797 |
| CII1631 | CDR | PRT | WO2016164637; SEQ ID NO: 484 | 3798 |
| CII1632 | CDR | PRT | US20190085084; SEQ ID NO: 514 | 3799 |
| CII1633 | CDR | PRT | WO2016164637; SEQ ID NO: 514 | 3800 |
| CII1634 | CDR | PRT | US20190085084; SEQ ID NO: 129 | 3801 |
| CII1635 | CDR | PRT | US20190085084; SEQ ID NO: 129 | 3802 |
| CII1636 | CDR | PRT | WO2016164637; SEQ ID NO: 129 | 3803 |
| CII1637 | CDR | PRT | US20190085084; SEQ ID NO: 483 | 3804 |
| CII1638 | CDR | PRT | US20190085084; SEQ ID NO: 483 | 3805 |
| CII1639 | CDR | PRT | WO2016164637; SEQ ID NO: 483 | 3806 |
| CII1640 | CDR | PRT | US20190085084; SEQ ID NO: 480 | 3807 |
| CII1641 | CDR | PRT | US20190085084; SEQ ID NO: 480 | 3808 |
| CII1642 | CDR | PRT | WO2016164637; SEQ ID NO: 480 | 3809 |
| CII1643 | CDR | PRT | US20190085084; SEQ ID NO: 518 | 3810 |
| CII1644 | CDR | PRT | US20190085084; SEQ ID NO: 518 | 3811 |
| CII1645 | CDR | PRT | WO2016164637; SEQ ID NO: 518 | 3812 |
| CII1646 | CDR | PRT | US20190085084; SEQ ID NO: 509 | 3813 |
| CII1647 | CDR | PRT | WO2016164637; SEQ ID NO: 509 | 3814 |
| CII1648 | CDR | PRT | WO2014028776; SEQ ID NO: 77 | 3815 |
| CII1649 | CDR | PRT | WO2014028776; SEQ ID NO: 80 | 3816 |
| CII1650 | CDR | PRT | WO2014028776; SEQ ID NO: 79 | 3817 |
| CII1651 | CDR | PRT | WO2014028776; SEQ ID NO: 78 | 3818 |
| CII1652 | CDR | PRT | WO2014028776; SEQ ID NO: 76 | 3819 |
| CII1653 | CDR | PRT | WO2014028776; SEQ ID NO: 81 | 3820 |
| CII1654 | CDR | PRT | WO2010129469; SEQ ID NO: 5 | 3821 |
| CII1655 | CDR | PRT | WO2010129469; SEQ ID NO: 6 | 3822 |
| CII1656 | CDR | PRT | WO2010129469; SEQ ID NO: 33 | 3823 |
| CII1657 | CDR | PRT | WO2010129469; SEQ ID NO: 34 | 3824 |
| CII1658 | CDR | PRT | WO2010129469; SEQ ID NO: 31 | 3825 |
| CII1659 | CDR | PRT | WO2010129469; SEQ ID NO: 30 | 3826 |
| CII1660 | CDR | PRT | WO2010129469; SEQ ID NO: 28 | 3827 |
| CII1661 | CDR | PRT | WO2010129469; SEQ ID NO: 27 | 3828 |
| CII1662 | CDR | PRT | WO2010129469; SEQ ID NO: 29 | 3829 |
| CII1663 | CDR | PRT | WO2010129469; SEQ ID NO: 32 | 3830 |
| CII1664 | CDR | PRT | WO2018140121; WO2018147919; SEQ | 3831 |
| | | | ID NO: 5 | |
| CII1665 | CDR | PRT | WO2018140121; WO2018147917; SEQ | 3832 |
| | | | ID NO: 3 | |
| CII1666 | CDR | PRT | WO2010129469; SEQ ID NO: 8 | 3833 |
| CII1667 | CDR | PRT | WO2010129469; SEQ ID NO: 24 | 3834 |
| CII1668 | CDR | PRT | WO2010129469; SEQ ID NO: 12 | 3835 |
| CII1669 | CDR | PRT | WO2010129469; SEQ ID NO: 20 | 3836 |
| CII1670 | CDR | PRT | WO2010129469; SEQ ID NO: 16 | 3837 |
| CII1671 | CDR | PRT | WO2010129469; SEQ ID NO: 25 | 3838 |
| CII1672 | CDR | PRT | WO2010129469; SEQ ID NO: 21 | 3839 |
| CII1673 | CDR | PRT | WO2010129469; SEQ ID NO: 11 | 3840 |
| CII1674 | CDR | PRT | WO2010129469; SEQ ID NO: 18 | 3841 |
| CII1675 | CDR | PRT | WO2010129469; SEQ ID NO: 15 | 3842 |
| CII1676 | CDR | PRT | WO2010129469; SEQ ID NO: 17 | 3843 |
| CII1677 | CDR | PRT | WO2010129469; SEQ ID NO: 23 | 3844 |
| CII1678 | CDR | PRT | WO2010129469; SEQ ID NO: 13 | 3845 |
| CII1679 | CDR | PRT | WO2010129469; SEQ ID NO: 14 | 3846 |
| CII1680 | CDR | PRT | WO2010129469; SEQ ID NO: 19 | 3847 |
| CII1681 | CDR | PRT | WO2018140121; WO2018147920; SEQ | 3848 |
| | | | ID NO: 6 | |
| CII1682 | CDR | PRT | WO2010129469; SEQ ID NO: 22 | 3849 |
| CII1683 | CDR | PRT | WO2010129469; SEQ ID NO: 26 | 3850 |
| CII1684 | CDR | PRT | WO2010129469; SEQ ID NO: 3 | 3851 |
| CII1685 | CDR | PRT | WO2010129469; SEQ ID NO: 7 | 3852 |
| CII1686 | CDR | PRT | WO2018140121; WO2018147918; SEQ | 3853 |
| | | | ID NO: 4 | |
| CII1687 | CDR | PRT | WO2018140121; WO2018147915; SEQ | 3854 |
| | | | ID NO: 1 | |
| CII1688 | CDR | PRT | WO2010129469; SEQ ID NO: 35 | 3855 |
| CII1689 | CDR | PRT | WO2010129469; SEQ ID NO: 4 | 3856 |
| CII1690 | CDR | PRT | WO2018140121; WO2018147916; SEQ | 3857 |
| | | | ID NO: 2 | |
| CII1691 | CDR | PRT | WO2017062672; SEQ ID NO: 741 | 3858 |
| CII1692 | CDR | PRT | WO2016023019; SEQ ID NO: 145 | 3859 |
| CII1693 | CDR | PRT | WO2017062672; SEQ ID NO: 31 | 3860 |
| CII1694 | CDR | PRT | WO2016023019; SEQ ID NO: 26 | 3861 |
| CII1695 | CDR | PRT | WO2016023019; SEQ ID NO: 118 | 3862 |
| CII1696 | CDR | PRT | WO2016023019; SEQ ID NO: 117 | 3863 |
| CII1697 | CDR | PRT | WO2016023019; SEQ ID NO: 70 | 3864 |
| CII1698 | CDR | PRT | WO2016023019; SEQ ID NO: 69 | 3865 |
| CII1699 | CDR | PRT | WO2016023019; SEQ ID NO: 50 | 3866 |
| CII1700 | CDR | PRT | WO2016023019; SEQ ID NO: 83 | 3867 |
| CII1701 | CDR | PRT | WO2016023019; SEQ ID NO: 71 | 3868 |
| CII1702 | CDR | PRT | WO2016023019; SEQ ID NO: 67 | 3869 |
| CII1703 | CDR | PRT | WO2016023019; SEQ ID NO: 103 | 3870 |
| CII1704 | CDR | PRT | WO2016023019; SEQ ID NO: 68 | 3871 |
| CII1705 | CDR | PRT | WO2016023019; SEQ ID NO: 86 | 3872 |
| CII1706 | CDR | PRT | WO2016023019; SEQ ID NO: 51 | 3873 |
| CII1707 | CDR | PRT | WO2016023019; SEQ ID NO: 85 | 3874 |
| CII1708 | CDR | PRT | WO2016023019; SEQ ID NO: 77 | 3875 |
| CII1709 | CDR | PRT | WO2016023019; SEQ ID NO: 102 | 3876 |
| CII1710 | CDR | PRT | WO2016023019; SEQ ID NO: 98 | 3877 |
| CII1711 | CDR | PRT | WO2016023019; SEQ ID NO: 406 | 3878 |
| CII1712 | CDR | PRT | WO2016023019; SEQ ID NO: 60 | 3879 |
| CII1713 | CDR | PRT | WO2016023019; SEQ ID NO: 107 | 3880 |
| CII1714 | CDR | PRT | WO2016023019; SEQ ID NO: 104 | 3881 |
| CII1715 | CDR | PRT | WO2016023019; SEQ ID NO: 84 | 3882 |
| CII1716 | CDR | PRT | WO2016023019; SEQ ID NO: 54 | 3883 |
| CII1717 | CDR | PRT | WO2016023019; SEQ ID NO: 96 | 3884 |
| CII1718 | CDR | PRT | WO2016023019; SEQ ID NO: 92 | 3885 |
| CII1719 | CDR | PRT | WO2016023019; SEQ ID NO: 65 | 3886 |
| CII1720 | CDR | PRT | WO2016023019; SEQ ID NO: 105 | 3887 |
| CII1721 | CDR | PRT | WO2016023019; SEQ ID NO: 108 | 3888 |
| CII1722 | CDR | PRT | WO2016023019; SEQ ID NO: 78 | 3889 |
| CII1723 | CDR | PRT | WO2016023019; SEQ ID NO: 114 | 3890 |
| CII1724 | CDR | PRT | WO2016023019; SEQ ID NO: 400 | 3891 |
| CII1725 | CDR | PRT | WO2016023019; SEQ ID NO: 59 | 3892 |
| CII1726 | CDR | PRT | WO2017062672; SEQ ID NO: 91 | 3893 |
| CII1727 | CDR | PRT | WO2017062672; SEQ ID NO: 763 | 3894 |
| CII1728 | CDR | PRT | WO2016023019; SEQ ID NO: 52 | 3895 |
| CII1729 | CDR | PRT | WO2016023019; SEQ ID NO: 82 | 3896 |
| CII1730 | CDR | PRT | WO2016023019; SEQ ID NO: 109 | 3897 |
| CII1731 | CDR | PRT | WO2016023019; SEQ ID NO: 113 | 3898 |
| CII1732 | CDR | PRT | WO2016023019; SEQ ID NO: 112 | 3899 |
| CII1733 | CDR | PRT | WO2017062672; SEQ ID NO: 86 | 3900 |
| CII1734 | CDR | PRT | WO2016023019; SEQ ID NO: 97 | 3901 |
| CII1735 | CDR | PRT | WO2016023019; SEQ ID NO: 95 | 3902 |
| CII1736 | CDR | PRT | WO2016023019; SEQ ID NO: 63 | 3903 |
| CII1737 | CDR | PRT | WO2016023019; SEQ ID NO: 87 | 3904 |
| CII1736 | CDR | PRT | WO2016023019; SEQ ID NO: 91 | 3905 |
| CII1739 | CDR | PRT | WO2016023019; SEQ ID NO: 110 | 3906 |
| CII1748 | CDR | PRT | WO2016023019; SEQ ID NO: 62 | 3907 |
| CII1741 | CDR | PRT | WO2016023019; SEQ ID NO: 93 | 3908 |
| CII1742 | CDR | PRT | WO2016023019; SEQ ID NO: 57 | 3909 |
| CII1743 | CDR | PRT | WO2016023019; SEQ ID NO: 73 | 3910 |
| CII1744 | CDR | PRT | WO2016023019; SEQ ID NO: 99 | 3911 |
| CII1745 | CDR | PRT | WO2016023019; SEQ ID NO: 55 | 3912 |
| CII1746 | CDR | PRT | WO2016023019; SEQ ID NO: 80 | 3913 |
| CII1747 | CDR | PRT | WO2016023019; SEQ ID NO: 53 | 3914 |
| CII1748 | CDR | PRT | WO2016023019; SEQ ID NO: 90 | 3915 |
| CII1749 | CDR | PRT | WO2016023019; SEQ ID NO: 64 | 3916 |
| CII1750 | CDR | PRT | WO2016023019; SEQ ID NO: 119 | 3917 |
| CII1751 | CDR | PRT | WO2017062672; SEQ ID NO: 709 | 3918 |
| CII1752 | CDR | PRT | WO2016023019; SEQ ID NO: 72 | 3919 |
| CII1753 | CDR | PRT | WO2016023019; SEQ ID NO: 61 | 3920 |
| CII1754 | CDR | PRT | WO2016023019; SEQ ID NO: 94 | 3921 |
| CII1755 | CDR | PRT | WO2016023019; SEQ ID NO: 74 | 3922 |
| CII1756 | CDR | PRT | WO2016023019; SEQ ID NO: 101 | 3923 |
| CII1757 | CDR | PRT | WO2016023019; SEQ ID NO: 100 | 3924 |
| CII1758 | CDR | PRT | WO2016023019; SEQ ID NO: 58 | 3925 |
| CII1759 | CDR | PRT | WO2019028292; SEQ ID NO: 138 | 3926 |
| CII1760 | CDR | PRT | WO2019028292; SEQ ID NO: 182 | 3927 |
| CII1761 | CDR | PRT | WO2019028292; SEQ ID NO: 183 | 3928 |
| CII1762 | CDR | PRT | WO2017062672; SEQ ID NO: 97 | 3929 |
| CII1763 | CDR | PRT | WO2019028292; SEQ ID NO: 126 | 3930 |
| CII1764 | CDR | PRT | WO2019028292; SEQ ID NO: 181 | 3931 |
| CII1765 | CDR | PRT | WO2019028292; SEQ ID NO: 123 | 3932 |
| CII1766 | CDR | PRT | WO2016023019; SEQ ID NO: 88 | 3933 |
| CII1767 | CDR | PRT | WO2016023019; SEQ ID NO: 89 | 3934 |
| CII1768 | CDR | PRT | WO2017062672; SEQ ID NO: 101 | 3935 |
| CII1769 | CDR | PRT | WO2016023019; SEQ ID NO: 75 | 3936 |
| CII1770 | CDR | PRT | WO2016023019; SEQ ID NO: 66 | 3937 |
| CII1771 | CDR | PRT | WO2016023019; SEQ ID NO: 106 | 3938 |
| CII1772 | CDR | PRT | WO2016023019; SEQ ID NO: 111 | 3939 |
| CII1773 | CDR | PRT | WO2016023019; SEQ ID NO: 79 | 3940 |
| CII1774 | CDR | PRT | WO2016023019; SEQ ID NO: 56 | 3941 |
| CII1775 | CDR | PRT | WO2016023019; SEQ ID NO: 81 | 3942 |
| CII1776 | CDR | PRT | WO2016023019; SEQ ID NO: 116 | 3943 |
| CII1777 | CDR | PRT | WO2016023019; SEQ ID NO: 76 | 3944 |
| CII1778 | CDR | PRT | WO2016023019; SEQ ID NO: 115 | 3945 |
| CII1779 | CDR | PRT | WO2017062672; SEQ ID NO: 767 | 3946 |
| CII1780 | CDR | PRT | WO2017062672; SEQ ID NO: 710 | 3947 |
| CII1781 | CDR | PRT | WO2017062672; SEQ ID NO: 770 | 3948 |
| CII1782 | CDR | PRT | WO2017062672; SEQ ID NO: 764 | 3949 |
| CII1783 | CDR | PRT | WO2016023019; SEQ ID NO: 142 | 3950 |
| CII1784 | CDR | PRT | WO2016023019; SEQ ID NO: 139 | 3951 |
| CII1785 | CDR | PRT | WO2016023019; SEQ ID NO: 146 | 3952 |
| CII1786 | CDR | PRT | WO2016023019; SEQ ID NO: 144 | 3953 |
| CII1787 | CDR | PRT | WO2016023019; SEQ ID NO: 149 | 3954 |
| CII1788 | CDR | PRT | WO2016023019; SEQ ID NO: 148 | 3955 |
| CII1789 | CDR | PRT | WO2017062672; SEQ ID NO: 84 | 3956 |
| CII1790 | CDR | PRT | WO2016023019; SEQ ID NO: 49 | 3957 |
| CII1791 | CDR | PRT | WO2017062672; SEQ ID NO: 73 | 3958 |
| CII1792 | CDR | PRT | WO2017062672; SEQ ID NO: 743 | 3959 |
| CII1793 | CDR | PRT | WO2017062672; SEQ ID NO: 62 | 3960 |
| CII1794 | CDR | PRT | WO2017062672; SEQ ID NO: 63 | 3961 |
| CII1795 | CDR | PRT | WO2017062672; SEQ ID NO: 699 | 3962 |
| CII1796 | CDR | PRT | WO2017062672; SEQ ID NO: 40 | 3963 |
| CII1797 | CDR | PRT | WO2017062672; SEQ ID NO: 65 | 3964 |
| CII1798 | CDR | PRT | WO2017062672; SEQ ID NO: 52 | 3965 |
| CII1799 | CDR | PRT | WO2017062672; SEQ ID NO: 57 | 3966 |
| CII1800 | CDR | PRT | WO2017062672; SEQ ID NO: 56 | 3967 |
| CII1801 | CDR | PRT | WO2017062672; SEQ ID NO: 51 | 3968 |
| CII1802 | CDR | PRT | WO2017062672; SEQ ID NO: 831 | 3969 |
| CII1803 | CDR | PRT | WO2016023019; SEQ ID NO: 4 | 3970 |
| CII1804 | CDR | PRT | WO2016023019; SEQ ID NO: 3 | 3971 |
| CII1805 | CDR | PRT | WO2016023019; SEQ ID NO: 16 | 3972 |
| CII1806 | CDR | PRT | WO2016023019; SEQ ID NO: 23 | 3973 |
| CII1807 | CDR | PRT | WO2016023019; SEQ ID NO: 18 | 3974 |
| CII1808 | CDR | PRT | WO2016023019; SEQ ID NO: 17 | 3975 |
| CII1809 | CDR | PRT | WO2017062672; SEQ ID NO: 753 | 3976 |
| CII1810 | CDR | PRT | WO2017062672; SEQ ID NO: 48 | 3977 |
| CII1811 | CDR | PRT | WO2017062672; SEQ ID NO: 32 | 3978 |
| CII1812 | CDR | PRT | WO2017062672; SEQ ID NO: 833 | 3979 |
| CII1813 | CDR | PRT | WO2017062672; SEQ ID NO: 832 | 3980 |
| CII1814 | CDR | PRT | WO2016023019; SEQ ID NO: 150 | 3981 |
| CII1815 | CDR | PRT | WO2016023019; SEQ ID NO: 408 | 3982 |
| CII1816 | CDR | PRT | WO2017062672; SEQ ID NO: 697 | 3983 |
| CII1817 | CDR | PRT | WO2016023019; SEQ ID NO: 143 | 3984 |
| CII1818 | CDR | PRT | WO2016023019; SEQ ID NO: 141 | 3985 |
| CII1819 | CDR | PRT | WO2016023019; SEQ ID NO: 138 | 3986 |
| CII1820 | CDR | PRT | WO2016023019; SEQ ID NO: 402 | 3987 |
| CII1821 | CDR | PRT | WO2016023019; SEQ ID NO: 27 | 3988 |
| CII1822 | CDR | PRT | WO2016023019; SEQ ID NO: 28 | 3989 |
| CII1823 | CDR | PRT | WO2017062672; SEQ ID NO: 702 | 3990 |
| CII1824 | CDR | PRT | WO2016023019; SEQ ID NO: 24 | 3991 |
| CII1825 | CDR | PRT | WO2016023019; SEQ ID NO: 13 | 3992 |
| CII1826 | CDR | PRT | WO2016023019; SEQ ID NO: 14 | 3993 |
| CII1827 | CDR | PRT | WO2016023019; SEQ ID NO: 8 | 3994 |
| CII1828 | CDR | PRT | WO2016023019; SEQ ID NO: 15 | 3995 |
| CII1829 | CDR | PRT | WO2016023019; SEQ ID NO: 5 | 3996 |
| CII1830 | CDR | PRT | WO2017062672; SEQ ID NO: 42 | 3997 |
| CII1831 | CDR | PRT | WO2017062672; SEQ ID NO: 700 | 3998 |
| CII1832 | CDR | PRT | WO2017062672; SEQ ID NO: 582 | 3999 |
| CII1833 | CDR | PRT | WO2017062672; SEQ ID NO: 70 | 4000 |
| CII1834 | CDR | PRT | WO2017062672; SEQ ID NO: 80 | 4001 |
| CII1835 | CDR | PRT | WO2017062672; SEQ ID NO: 755 | 4002 |
| CII1836 | CDR | PRT | WO2017062672; SEQ ID NO: 81 | 4003 |
| CII1837 | CDR | PRT | WO2017062672; SEQ ID NO: 72 | 4004 |
| CII1838 | CDR | PRT | WO2017062672; SEQ ID NO: 586 | 4005 |
| CII1839 | CDR | PRT | WO2017062672; SEQ ID NO: 67 | 4006 |
| CII1840 | CDR | PRT | WO2017062672; SEQ ID NO: 77 | 4007 |
| CII1841 | CDR | PRT | WO2017062672; SEQ ID NO: 757 | 4008 |
| CII1842 | CDR | PRT | WO2017062672; SEQ ID NO: 79 | 4009 |
| CII1843 | CDR | PRT | WO2017062672; SEQ ID NO: 71 | 4010 |
| CII1844 | CDR | PRT | WO2017062672; SEQ ID NO: 758 | 4011 |
| CII1845 | CDR | PRT | WO2017062672; SEQ ID NO: 78 | 4012 |
| CII1846 | CDR | PRT | WO2017062672; SEQ ID NO: 76 | 4013 |
| CII1847 | CDR | PRT | WO2017062672; SEQ ID NO: 756 | 4014 |
| CII1848 | CDR | PRT | WO2017062672; SEQ ID NO: 587 | 4015 |
| CII1849 | CDR | PRT | WO2017062672; SEQ ID NO: 68 | 4016 |
| CII1850 | CDR | PRT | WO2017062672; SEQ ID NO: 841 | 4017 |
| CII1851 | CDR | PRT | WO2017062672; SEQ ID NO: 842 | 4018 |
| CII1852 | CDR | PRT | WO2017062672; SEQ ID NO: 837 | 4019 |
| CII1853 | CDR | PRT | WO2017062672; SEQ ID NO: 836 | 4020 |
| CII1854 | CDR | PRT | WO2017062672; SEQ ID NO: 762 | 4021 |
| CII1855 | CDR | PRT | WO2017062672; SEQ ID NO: 760 | 4022 |
| CII1856 | CDR | PRT | WO2017062672; SEQ ID NO: 759 | 4023 |
| CII1857 | CDR | PRT | WO2017062672; SEQ ID NO: 83 | 4024 |
| CII1858 | CDR | PRT | WO2016023019; SEQ ID NO: 30 | 4025 |
| CII1859 | CDR | PRT | WO2016023019; SEQ ID NO: 29 | 4026 |
| CII1860 | CDR | PRT | WO2016023019; SEQ ID NO: 399 | 4027 |
| CII1861 | CDR | PRT | WO2016023019; SEQ ID NO: 31 | 4028 |
| CII1862 | CDR | PRT | WO2016023019; SEQ ID NO: 34 | 4029 |
| CII1863 | CDR | PRT | WO2016023019; SEQ ID NO: 405 | 4030 |
| CII1864 | CDR | PRT | WO2016023019; SEQ ID NO: 33 | 4031 |
| CII1865 | CDR | PRT | WO2017062672; SEQ ID NO: 30 | 4032 |
| CII1866 | CDR | PRT | WO2016023019; SEQ ID NO: 152 | 4033 |
| CII1867 | CDR | PRT | WO2017062672; SEQ ID NO: 21 | 4034 |
| CII1868 | CDR | PRT | WO2017062672; SEQ ID NO: 745 | 4035 |
| CII1869 | CDR | PRT | WO2017062672; SEQ ID NO: 12 | 4036 |
| CII1870 | CDR | PRT | WO2017062672; SEQ ID NO: 22 | 4037 |
| CII1871 | CDR | PRT | WO2017062672; SEQ ID NO: 23 | 4038 |
| CII1872 | CDR | PRT | WO2017062672; SEQ ID NO: 11 | 4039 |
| CII1873 | CDR | PRT | WO2017062672; SEQ ID NO: 16 | 4040 |
| CII1874 | CDR | PRT | WO2017062672; SEQ ID NO: 10 | 4041 |
| CII1875 | CDR | PRT | WO2017062672; SEQ ID NO: 581 | 4042 |
| CII1876 | CDR | PRT | WO2016023019; SEQ ID NO: 127 | 4043 |
| CII1877 | CDR | PRT | WO2017062672; SEQ ID NO: 828 | 4044 |
| CII1878 | CDR | PRT | WO2017062672; SEQ ID NO: 740 | 4045 |
| CII1879 | CDR | PRT | WO2017062672; SEQ ID NO: 33 | 4046 |
| CII1880 | CDR | PRT | WO2017062672; SEQ ID NO: 28 | 4047 |
| CII1881 | CDR | PRT | WO2019028292; SEQ ID NO: 131 | 4048 |
| CII1882 | CDR | PRT | WO2019028292; SEQ ID NO: 193 | 4049 |
| CII1883 | CDR | PRT | WO2019028292; SEQ ID NO: 134 | 4050 |
| CII1884 | CDR | PRT | WO2017062672; SEQ ID NO: 739 | 4051 |
| CII1885 | CDR | PRT | WO2019028292; SEQ ID NO: 140 | 4052 |
| CII1886 | CDR | PRT | WO2019028292; SEQ ID NO: 128 | 4053 |
| CII1887 | CDR | PRT | WO2016023019; SEQ ID NO: 208 | 4054 |
| CII1888 | CDR | PRT | WO2016023019; SEQ ID NO: 151 | 4055 |
| CII1889 | CDR | PRT | WO2017062672; SEQ ID NO: 707 | 4056 |
| CII1890 | CDR | PRT | WO2016023019; SEQ ID NO: 40 | 4057 |
| CII1891 | CDR | PRT | WO2019028292; SEQ ID NO: 197 | 4058 |
| CII1892 | CDR | PRT | WO2019028292; SEQ ID NO: 195 | 4059 |
| CII1893 | CDR | PRT | WO2017062672; SEQ ID NO: 27 | 4060 |
| CII1894 | CDR | PRT | WO2017062672; SEQ ID NO: 55 | 4061 |
| CII1895 | CDR | PRT | WO2017062672; SEQ ID NO: 26 | 4062 |
| CII1896 | CDR | PRT | WO2016023019; SEQ ID NO: 228 | 4063 |
| CII1897 | CDR | PRT | WO2017062672; SEQ ID NO: 36 | 4064 |
| CII1898 | CDR | PRT | WO2017062672; SEQ ID NO: 742 | 4065 |
| CII1899 | CDR | PRT | WO2016023019; SEQ ID NO: 36 | 4066 |
| CII1900 | CDR | PRT | WO2017062672; SEQ ID NO: 24 | 4067 |
| CII1901 | CDR | PRT | WO2016023019; SEQ ID NO: 128 | 4068 |
| CII1902 | CDR | PRT | WO2016023019; SEQ ID NO: 122 | 4069 |
| CII1903 | CDR | PRT | WO2016023019; SEQ ID NO: 133 | 4070 |
| CII1904 | CDR | PRT | WO2017062672; SEQ ID NO: 41 | 4071 |
| CII1905 | CDR | PRT | WO2017062672; SEQ ID NO: 34 | 4072 |
| CII1906 | CDR | PRT | WO2017062672; SEQ ID NO: 46 | 4073 |
| CII1907 | CDR | PRT | WO2016023019; SEQ ID NO: 227 | 4074 |
| CII1908 | CDR | PRT | WO2016023019; SEQ ID NO: 211 | 4075 |
| CII1909 | CDR | PRT | WO2016023019; SEQ ID NO: 175 | 4076 |
| CII1910 | CDR | PRT | WO2016023019; SEQ ID NO: 220 | 4077 |
| CII1911 | CDR | PRT | WO2016023019; SEQ ID NO: 231 | 4078 |
| CII1912 | CDR | PRT | WO2016023019; SEQ ID NO: 174 | 4079 |
| CII1913 | CDR | PRT | WO2016023019; SEQ ID NO: 213 | 4080 |
| CII1914 | CDR | PRT | WO2016023019; SEQ ID NO: 226 | 4081 |
| CII1915 | CDR | PRT | WO2016023019; SEQ ID NO: 214 | 4082 |
| CII1916 | CDR | PRT | WO2016023019; SEQ ID NO: 222 | 4083 |
| CII1917 | CDR | PRT | WO2016023019; SEQ ID NO: 176 | 4084 |
| CII1918 | CDR | PRT | WO2016023019; SEQ ID NO: 173 | 4085 |
| CII1919 | CDR | PRT | WO2016023019; SEQ ID NO: 409 | 4086 |
| CII1920 | CDR | PRT | WO2016023019; SEQ ID NO: 156 | 4087 |
| CII1921 | CDR | PRT | WO2016023019; SEQ ID NO: 184 | 4088 |
| CII1922 | CDR | PRT | WO2016023019; SEQ ID NO: 221 | 4089 |
| CII1923 | CDR | PRT | WO2016023019; SEQ ID NO: 215 | 4090 |
| CII1924 | CDR | PRT | WO2016023019; SEQ ID NO: 163 | 4091 |
| CII1925 | CDR | PRT | WO2016023019; SEQ ID NO: 177 | 4092 |
| CII1926 | CDR | PRT | WO2016023019; SEQ ID NO: 234 | 4093 |
| CII1927 | CDR | PRT | WO2016023019; SEQ ID NO: 164 | 4094 |
| CII1928 | CDR | PRT | WO2016023019; SEQ ID NO: 202 | 4095 |
| CII1929 | CDR | PRT | WO2016023019; SEQ ID NO: 167 | 4096 |
| CII1930 | CDR | PRT | WO2016023019; SEQ ID NO: 169 | 4097 |
| CII1931 | CDR | PRT | WO2016023019; SEQ ID NO: 170 | 4098 |
| CII1932 | CDR | PRT | WO2016023019; SEQ ID NO: 168 | 4099 |
| CII1933 | CDR | PRT | WO2016023019; SEQ ID NO: 172 | 4100 |
| CII1934 | CDR | PRT | WO2016023019; SEQ ID NO: 161 | 4101 |
| CII1935 | CDR | PRT | WO2016023019; SEQ ID NO: 191 | 4102 |
| CII1936 | CDR | PRT | WO2016023019; SEQ ID NO: 186 | 4103 |
| CII1937 | CDR | PRT | WO2016023019; SEQ ID NO: 162 | 4104 |
| CII1938 | CDR | PRT | WO2016023019; SEQ ID NO: 209 | 4105 |
| CII1939 | CDR | PRT | WO2017062672; SEQ ID NO: 701 | 4106 |
| CII1940 | CDR | PRT | WO2017062672; SEQ ID NO: 698 | 4107 |
| CII1941 | CDR | PRT | WO2016023019; SEQ ID NO: 160 | 4108 |
| CII1942 | CDR | PRT | WO2016023019; SEQ ID NO: 189 | 4109 |
| CII1943 | CDR | PRT | WO2016023019; SEQ ID NO: 196 | 4110 |
| CII1944 | CDR | PRT | WO2016023019; SEQ ID NO: 187 | 4111 |
| CII1945 | CDR | PRT | WO2016023019; SEQ ID NO: 235 | 4112 |
| CII1946 | CDR | PRT | WO2016023019; SEQ ID NO: 153 | 4113 |
| CII1947 | CDR | PRT | WO2016023019; SEQ ID NO: 205 | 4114 |
| CII1948 | CDR | PRT | WO2016023019; SEQ ID NO: 195 | 4115 |
| CII1949 | CDR | PRT | WO2017062672; SEQ ID NO: 744 | 4116 |
| CII1950 | CDR | PRT | WO2016023019; SEQ ID NO: 155 | 4117 |
| CII1951 | CDR | PRT | WO2016023019; SEQ ID NO: 180 | 4118 |
| CII1952 | CDR | PRT | WO2016023019; SEQ ID NO: 182 | 4119 |
| CII1953 | CDR | PRT | WO2016023019; SEQ ID NO: 199 | 4120 |
| CII1954 | CDR | PRT | WO2016023019; SEQ ID NO: 219 | 4121 |
| CII1955 | CDR | PRT | WO2016023019; SEQ ID NO: 188 | 4122 |
| CII1956 | CDR | PRT | WO2016023019; SEQ ID NO: 218 | 4123 |
| CII1957 | CDR | PRT | WO2016023019; SEQ ID NO: 210 | 4124 |
| CII1958 | CDR | PRT | WO2016023019; SEQ ID NO: 198 | 4125 |
| CII1959 | CDR | PRT | WO2016023019; SEQ ID NO: 159 | 4126 |
| CII1960 | CDR | PRT | WO2016023019; SEQ ID NO: 232 | 4127 |
| CII1961 | CDR | PRT | WO2016023019; SEQ ID NO: 216 | 4128 |
| CII1962 | CDR | PRT | WO2016023019; SEQ ID NO: 217 | 4129 |
| CII1963 | CDR | PRT | WO2016023019; SEQ ID NO: 197 | 4130 |
| CII1964 | CDR | PRT | WO2016023019; SEQ ID NO: 223 | 4131 |
| CII1965 | CDR | PRT | WO2016023019; SEQ ID NO: 206 | 4132 |
| CII1966 | CDR | PRT | WO2016023019; SEQ ID NO: 200 | 4133 |
| CII1967 | CDR | PRT | WO2016023019; SEQ ID NO: 158 | 4134 |
| CII1968 | CDR | PRT | WO2016023019; SEQ ID NO: 192 | 4135 |
| CII1969 | CDR | PRT | WO2016023019; SEQ ID NO: 179 | 4136 |
| CII1970 | CDR | PRT | WO2016023019; SEQ ID NO: 204 | 4137 |
| CII1971 | CDR | PRT | WO2016023019; SEQ ID NO: 185 | 4138 |
| CII1972 | CDR | PRT | WO2016023019; SEQ ID NO: 230 | 4139 |
| CII1973 | CDR | PRT | WO2016023019; SEQ ID NO: 212 | 4140 |
| CII1974 | CDR | PRT | WO2016023019; SEQ ID NO: 233 | 4141 |
| CII1975 | CDR | PRT | WO2017062672; SEQ ID NO: 746 | 4142 |
| CII1976 | CDR | PRT | WO2016023019; SEQ ID NO: 166 | 4143 |
| CII1977 | CDR | PRT | WO2016023019; SEQ ID NO: 403 | 4144 |
| CII1978 | CDR | PRT | WO2016023019; SEQ ID NO: 207 | 4145 |
| CII1979 | CDR | PRT | WO2016023019; SEQ ID NO: 224 | 4146 |
| CII1980 | CDR | PRT | WO2016023019; SEQ ID NO: 165 | 4147 |
| CII1981 | CDR | PRT | WO2016023019; SEQ ID NO: 181 | 4148 |
| CII1982 | CDR | PRT | WO2016023019; SEQ ID NO: 190 | 4149 |
| CII1983 | CDR | PRT | WO2016023019; SEQ ID NO: 194 | 4150 |
| CII1984 | CDR | PRT | WO2017062672; SEQ ID NO: 38 | 4151 |
| CII1985 | CDR | PRT | WO2016023019; SEQ ID NO: 201 | 4152 |
| CII1986 | CDR | PRT | WO2016023019; SEQ ID NO: 193 | 4153 |
| CII1987 | CDR | PRT | WO2016023019; SEQ ID NO: 154 | 4154 |
| CII1988 | CDR | PRT | WO2016023019; SEQ ID NO: 157 | 4155 |
| CII1989 | CDR | PRT | WO2016023019; SEQ ID NO: 225 | 4156 |
| CII1990 | CDR | PRT | WO2016023019; SEQ ID NO: 236 | 4157 |
| CII1991 | CDR | PRT | WO2016023019; SEQ ID NO: 178 | 4158 |
| CII1992 | CDR | PRT | WO2016023019; SEQ ID NO: 183 | 4159 |
| CII1993 | CDR | PRT | WO2016023019; SEQ ID NO: 203 | 4160 |
| CII1994 | CDR | PRT | WO2016023019; SEQ ID NO: 229 | 4161 |
| CII1995 | CDR | PRT | WO2016023019; SEQ ID NO: 171 | 4162 |
| CII1996 | CDR | PRT | WO2017062672; SEQ ID NO: 47 | 4163 |
| CII1997 | CDR | PRT | WO2017062672; SEQ ID NO: 35 | 4164 |
| CII1998 | CDR | PRT | WO2017062672; SEQ ID NO: 9 | 4165 |
| CII1999 | CDR | PRT | WO2017062672; SEQ ID NO: 17 | 4166 |
| CII2000 | CDR | PRT | WO2016023019; SEQ ID NO: 136 | 4167 |
| CII2001 | CDR | PRT | WO2016023019; SEQ ID NO: 132 | 4168 |
| CII2002 | CDR | PRT | WO2016023019; SEQ ID NO: 135 | 4169 |
| CII2003 | CDR | PRT | WO2017062672; SEQ ID NO: 738 | 4170 |
| CII2004 | CDR | PRT | WO2016023019; SEQ ID NO: 130 | 4171 |
| CII2005 | CDR | PRT | WO2016023019; SEQ ID NO: 129 | 4172 |
| CII2006 | CDR | PRT | WO2016023019; SEQ ID NO: 131 | 4173 |
| CII2007 | CDR | PRT | WO2016023019; SEQ ID NO: 125 | 4174 |
| CII2008 | CDR | PRT | WO2016023019; SEQ ID NO: 121 | 4175 |
| CII2009 | CDR | PRT | WO2016023019; SEQ ID NO: 137 | 4176 |
| CII2010 | CDR | PRT | WO2016023019; SEQ ID NO: 126 | 4177 |
| CII2011 | CDR | PRT | WO2016023019; SEQ ID NO: 120 | 4178 |
| CII2012 | CDR | PRT | WO2016023019; SEQ ID NO: 401 | 4179 |
| CII2013 | CDR | PRT | WO2016023019; SEQ ID NO: 123 | 4180 |
| CII2014 | CDR | PRT | WO2016023019; SEQ ID NO: 407 | 4181 |
| CII2015 | CDR | PRT | WO2016023019; SEQ ID NO: 124 | 4182 |
| CII2016 | CDR | PRT | WO2017062672; SEQ ID NO: 20 | 4183 |
| CII2017 | CDR | PRT | WO2019028292; SEQ ID NO: 167 | 4184 |
| CII2018 | CDR | PRT | WO2019028292; SEQ ID NO: 168 | 4185 |
| CII2019 | CDR | PRT | WO2019028292; SEQ ID NO: 125 | 4186 |
| CII2020 | CDR | PRT | WO2019028292; SEQ ID NO: 165 | 4187 |
| CII2021 | CDR | PRT | WO2019028292; SEQ ID NO: 169 | 4188 |
| CII2022 | CDR | PRT | WO2019028292; SEQ ID NO: 170 | 4189 |
| CII2023 | CDR | PRT | WO2019028292; SEQ ID NO: 166 | 4190 |
| CII2024 | CDR | PRT | WO2019028292; SEQ ID NO: 171 | 4191 |
| CII2025 | CDR | PRT | WO2019028292; SEQ ID NO: 172 | 4192 |
| CII2026 | CDR | PRT | WO2017062672; SEQ ID NO: 888 | 4193 |
| CII2027 | CDR | PRT | WO2019028292; SEQ ID NO: 173 | 4194 |
| CII2028 | CDR | PRT | WO2019028292; SEQ ID NO: 143 | 4195 |
| CII2029 | CDR | PRT | WO2019028292; SEQ ID NO: 179 | 4196 |
| CII2030 | CDR | PRT | WO2019028292; SEQ ID NO: 163 | 4197 |
| CII2031 | CDR | PRT | WO2019028292; SEQ ID NO: 177 | 4198 |
| CII2032 | CDR | PRT | WO2019028292; SEQ ID NO: 137 | 4199 |
| CII2033 | CDR | PRT | WO2019028292; SEQ ID NO: 141 | 4200 |
| CII2034 | CDR | PRT | WO2019028292; SEQ ID NO: 180 | 4201 |
| CII2035 | CDR | PRT | WO2019028292; SEQ ID NO: 135 | 4202 |
| CII2036 | CDR | PRT | WO2019028292; SEQ ID NO: 162 | 4203 |
| CII2037 | CDR | PRT | WO2019028292; SEQ ID NO: 174 | 4204 |
| CII2038 | CDR | PRT | WO2019028292; SEQ ID NO: 175 | 4205 |
| CII2039 | CDR | PRT | WO2019028292; SEQ ID NO: 133 | 4206 |
| CII2040 | CDR | PRT | WO2019028292; SEQ ID NO: 178 | 4207 |
| CII2041 | CDR | PRT | WO2019028292; SEQ ID NO: 164 | 4208 |
| CII2042 | CDR | PRT | WO2019028292; SEQ ID NO: 176 | 4209 |
| CII2043 | CDR | PRT | WO2019028292; SEQ ID NO: 122 | 4210 |
| CII2044 | CDR | PRT | WO2017062672; SEQ ID NO: 88 | 4211 |
| CII2045 | CDR | PRT | WO2019028292; SEQ ID NO: 185 | 4212 |
| CII2046 | CDR | PRT | WO2016023019; SEQ ID NO: 134 | 4213 |
| CII2047 | CDR | PRT | WO2017062672; SEQ ID NO: 14 | 4214 |
| CII2048 | CDR | PRT | WO2017062672; SEQ ID NO: 19 | 4215 |
| CII2049 | CDR | PRT | WO2019028292; SEQ ID NO: 130 | 4216 |
| CII2050 | CDR | PRT | WO2019028292; SEQ ID NO: 142 | 4217 |
| CII2051 | CDR | PRT | WO2019028292; SEQ ID NO: 144 | 4218 |
| CII2052 | CDR | PRT | WO2019028292; SEQ ID NO: 184 | 4219 |
| CII2053 | CDR | PRT | WO2017062672; SEQ ID NO: 15 | 4220 |
| CII2054 | CDR | PRT | WO2017062672; SEQ ID NO: 827 | 4221 |
| CII2055 | CDR | PRT | WO2019028292; SEQ ID NO: 187 | 4222 |
| CII2056 | CDR | PRT | WO2019028292; SEQ ID NO: 190 | 4223 |
| CII2057 | CDR | PRT | WO2019028292; SEQ ID NO: 189 | 4224 |
| CII2058 | CDR | PRT | WO2019028292; SEQ ID NO: 188 | 4225 |
| CII2059 | CDR | PRT | WO2017062672; SEQ ID NO: 18 | 4226 |
| CII2060 | CDR | PRT | WO2019028292; SEQ ID NO: 139 | 4227 |
| CII2061 | CDR | PRT | WO2019028292; SEQ ID NO: 186 | 4228 |
| CII2062 | CDR | PRT | WO2019028292; SEQ ID NO: 192 | 4229 |
| CII2063 | CDR | PRT | WO2019028292; SEQ ID NO: 191 | 4230 |
| CII2064 | CDR | PRT | WO2017062672; SEQ ID NO: 826 | 4231 |
| CII2065 | CDR | PRT | WO2019028292; SEQ ID NO: 127 | 4232 |
| CII2066 | CDR | PRT | WO2017062672; SEQ ID NO: 704 | 4233 |
| CII2067 | CDR | PRT | WO2017062672; SEQ ID NO: 13 | 4234 |
| CII2068 | CDR | PRT | WO2016023019; SEQ ID NO: 140 | 4235 |
| CII2069 | CDR | PRT | WO2017062672; SEQ ID NO: 695 | 4236 |
| CII2070 | CDR | PRT | WO2019028292; SEQ ID NO: 196 | 4237 |
| CII2071 | CDR | PRT | WO2017062672; SEQ ID NO: 737 | 4238 |
| CII2072 | CDR | PRT | WO2017062672; SEQ ID NO: 694 | 4239 |
| CII2073 | CDR | PRT | WO2017062672; SEQ ID NO: 736 | 4240 |
| CII2074 | CDR | PRT | WO2017062672; SEQ ID NO: 45 | 4241 |
| CII2075 | CDR | PRT | WO2017062672; SEQ ID NO: 749 | 4242 |
| CII2076 | CDR | PRT | WO2017062672; SEQ ID NO: 748 | 4243 |
| CII2077 | CDR | PRT | WO2017062672; SEQ ID NO: 747 | 4244 |
| CII2078 | CDR | PRT | WO2017062672; SEQ ID NO: 750 | 4245 |
| CII2079 | CDR | PRT | WO2017062672; SEQ ID NO: 751 | 4246 |
| CII2080 | CDR | PRT | WO2016023019; SEQ ID NO: 43 | 4247 |
| CII2081 | CDR | PRT | WO2016023019; SEQ ID NO: 46 | 4248 |
| CII2082 | CDR | PRT | WO2016023019; SEQ ID NO: 32 | 4249 |
| CII2083 | CDR | PRT | WO2016023019; SEQ ID NO: 38 | 4250 |
| CII2084 | CDR | PRT | WO2016023019; SEQ ID NO: 37 | 4251 |
| CII2085 | CDR | PRT | WO2017062672; SEQ ID NO: 693 | 4252 |
| CII2086 | CDR | PRT | WO2017062672; SEQ ID NO: 690 | 4253 |
| CII2087 | CDR | PRT | WO2017062672; SEQ ID NO: 692 | 4254 |
| CII2088 | CDR | PRT | WO2017062672; SEQ ID NO: 691 | 4255 |
| CII2089 | CDR | PRT | WO2017062672; SEQ ID NO: 735 | 4256 |
| CII2090 | CDR | PRT | WO2017062672; SEQ ID NO: 734 | 4257 |
| CII2091 | CDR | PRT | WO2017062672; SEQ ID NO: 39 | 4258 |
| CII2092 | CDR | PRT | WO2017062672; SEQ ID NO: 583 | 4259 |
| CII2093 | CDR | PRT | WO2017062672; SEQ ID NO: 44 | 4260 |
| CII2094 | CDR | PRT | WO2017062672; SEQ ID NO: 43 | 4261 |
| CII2095 | CDR | PRT | WO2019028292; SEQ ID NO: 129 | 4262 |
| CII2096 | CDR | PRT | WO2019028292; SEQ ID NO: 194 | 4263 |
| CII2097 | CDR | PRT | WO2017062672; SEQ ID NO: 96 | 4264 |
| CII2098 | CDR | PRT | WO2017062672; SEQ ID NO: 703 | 4265 |
| CII2099 | CDR | PRT | WO2017062672; SEQ ID NO: 705 | 4266 |
| CII2100 | CDR | PRT | WO2017062672; SEQ ID NO: 769 | 4267 |
| CII2101 | CDR | PRT | WO2017062672; SEQ ID NO: 768 | 4268 |
| CII2102 | CDR | PRT | WO2017062672; SEQ ID NO: 85 | 4269 |
| CII2103 | CDR | PRT | WO2017062672; SEQ ID NO: 588 | 4270 |
| CII2104 | CDR | PRT | WO2017062672; SEQ ID NO: 93 | 4271 |
| CII2105 | CDR | PRT | WO2017062672; SEQ ID NO: 87 | 4272 |
| CII2106 | CDR | PRT | WO2017062672; SEQ ID NO: 706 | 4273 |
| CII2107 | CDR | PRT | WO2017062672; SEQ ID NO: 69 | 4274 |
| CII2108 | CDR | PRT | WO2017062672; SEQ ID NO: 585 | 4275 |
| CII2109 | CDR | PRT | WO2017062672; SEQ ID NO: 74 | 4276 |
| CII2110 | CDR | PRT | WO2017062672; SEQ ID NO: 838 | 4277 |
| CII2111 | CDR | PRT | WO2017062672; SEQ ID NO: 75 | 4278 |
| CII2112 | CDR | PRT | WO2017062672; SEQ ID NO: 761 | 4279 |
| CII2113 | CDR | PRT | WO2017062672; SEQ ID NO: 82 | 4280 |
| CII2114 | CDR | PRT | WO2017062672; SEQ ID NO: 66 | 4281 |
| CII2115 | CDR | PRT | WO2017062672; SEQ ID NO: 708 | 4282 |
| CII2116 | CDR | PRT | WO2017062672; SEQ ID NO: 99 | 4283 |
| CII2117 | CDR | PRT | WO2016023019; SEQ ID NO: 25 | 4284 |
| CII2118 | CDR | PRT | WO2016023019; SEQ ID NO: 39 | 4285 |
| CII2119 | CDR | PRT | WO2016023019; SEQ ID NO: 41 | 4286 |
| CII2120 | CDR | PRT | WO2017062672; SEQ ID NO: 766 | 4287 |
| CII2121 | CDR | PRT | WO2017062672; SEQ ID NO: 589 | 4288 |
| CII2122 | CDR | PRT | WO2017062672; SEQ ID NO: 90 | 4289 |
| CII2123 | CDR | PRT | WO2017062672; SEQ ID NO: 98 | 4290 |
| CII2124 | CDR | PRT | WO2017062672; SEQ ID NO: 89 | 4291 |
| CII2125 | CDR | PRT | WO2017062672; SEQ ID NO: 102 | 4292 |
| CII2126 | CDR | PRT | WO2017062672; SEQ ID NO: 92 | 4293 |
| CII2127 | CDR | PRT | WO2017062672; SEQ ID NO: 100 | 4294 |
| CII2128 | CDR | PRT | WO2017062672; SEQ ID NO: 95 | 4295 |
| CII2129 | CDR | PRT | WO2017062672; SEQ ID NO: 765 | 4296 |
| CII2130 | CDR | PRT | WO2017062672; SEQ ID NO: 94 | 4297 |
| CII2131 | CDR | PRT | WO2017062672; SEQ ID NO: 25 | 4298 |
| CII2132 | CDR | PRT | WO2016023019; SEQ ID NO: 147 | 4299 |
| CII2133 | CDR | PRT | WO2017062672; SEQ ID NO: 29 | 4300 |
| CII2134 | CDR | PRT | WO2017062672; SEQ ID NO: 696 | 4301 |
| CII2135 | CDR | PRT | WO2016023019; SEQ ID NO: 42 | 4302 |
| CII2136 | CDR | PRT | WO2016023019; SEQ ID NO: 44 | 4303 |
| CII2137 | CDR | PRT | WO2016023019; SEQ ID NO: 35 | 4304 |
| CII2138 | CDR | PRT | WO2016023019; SEQ ID NO: 45 | 4305 |
| CII2139 | CDR | PRT | WO2017062672; SEQ ID NO: 37 | 4306 |
| CII2140 | CDR | PRT | WO2017062672; SEQ ID NO: 840 | 4307 |
| CII2141 | CDR | PRT | WO2017062672; SEQ ID NO: 839 | 4308 |
| CII2142 | CDR | PRT | WO2019028292; SEQ ID NO: 157 | 4309 |
| CII2143 | CDR | PRT | WO2017062672; SEQ ID NO: 59 | 4310 |
| CII2144 | CDR | PRT | WO2019028292; SEQ ID NO: 158 | 4311 |
| CII2145 | CDR | PRT | WO2019028292; SEQ ID NO: 136 | 4312 |
| CII2146 | CDR | PRT | WO2019028292; SEQ ID NO: 160 | 4313 |
| CII2147 | CDR | PRT | WO2019028292; SEQ ID NO: 159 | 4314 |
| CII2148 | CDR | PRT | WO2019028292; SEQ ID NO: 132 | 4315 |
| CII2149 | CDR | PRT | WO2017062672; SEQ ID NO: 60 | 4316 |
| CII2150 | CDR | PRT | WO2019028292; SEQ ID NO: 124 | 4317 |
| CII2151 | CDR | PRT | WO2019028292; SEQ ID NO: 161 | 4318 |
| CII2152 | CDR | PRT | WO2019028292; SEQ ID NO: 121 | 4319 |
| CII2153 | CDR | PRT | WO2017062672; SEQ ID NO: 61 | 4320 |
| CII2154 | CDR | PRT | WO2016023019; SEQ ID NO: 47 | 4321 |
| CII2155 | CDR | PRT | WO2016023019; SEQ ID NO: 48 | 4322 |
| CII2156 | CDR | PRT | WO2017062672; SEQ ID NO: 54 | 4323 |
| CII2157 | CDR | PRT | WO2017062672; SEQ ID NO: 752 | 4324 |
| CII2158 | CDR | PRT | WO2017062672; SEQ ID NO: 584 | 4325 |
| CII2159 | CDR | PRT | WO2016023019; SEQ ID NO: 10 | 4326 |
| CII2160 | CDR | PRT | WO2016023019; SEQ ID NO: 9 | 4327 |
| CII2161 | CDR | PRT | WO2016023019; SEQ ID NO: 11 | 4328 |
| CII2162 | CDR | PRT | WO2016023019; SEQ ID NO: 404 | 4329 |
| CII2163 | CDR | PRT | WO2017062672; SEQ ID NO: 64 | 4330 |
| CII2164 | CDR | PRT | WO2017062672; SEQ ID NO: 50 | 4331 |
| CII2165 | CDR | PRT | WO2017062672; SEQ ID NO: 754 | 4332 |
| CII2166 | CDR | PRT | WO2017062672; SEQ ID NO: 49 | 4333 |
| CII2167 | CDR | PRT | WO2016023019; SEQ ID NO: 19 | 4334 |
| CII2168 | CDR | PRT | WO2016023019; SEQ ID NO: 20 | 4335 |
| CII2169 | CDR | PRT | WO2016023019; SEQ ID NO: 21 | 4336 |
| CII2170 | CDR | PRT | WO2016023019; SEQ ID NO: 22 | 4337 |
| CII2171 | CDR | PRT | WO2016023019; SEQ ID NO: 12 | 4338 |
| CII2172 | CDR | PRT | WO2016023019; SEQ ID NO: 398 | 4339 |
| CII2173 | CDR | PRT | WO2016023019; SEQ ID NO: 6 | 4340 |
| CII2174 | CDR | PRT | WO2016023019; SEQ ID NO: 7 | 4341 |
| CII2175 | CDR | PRT | WO2017062672; SEQ ID NO: 58 | 4342 |
| CII2176 | CDR | PRT | WO2017062672; SEQ ID NO: 830 | 4343 |
| CII2177 | CDR | PRT | WO2017062672; SEQ ID NO: 53 | 4344 |
| CII2178 | CDR | PRT | WO2017062672; SEQ ID NO: 835 | 4345 |
| CII2179 | CDR | PRT | WO2017062672; SEQ ID NO: 834 | 4346 |
| CII2180 | CDR | PRT | WO2017062672; SEQ ID NO: 829 | 4347 |
| CII2181 | CDR | PRT | WO2014028776; SEQ ID NO: 69 | 4348 |
| CII2182 | CDR | PRT | WO2014028776; SEQ ID NO: 71 | 4349 |
| CII2183 | CDR | PRT | WO2014028776; SEQ ID NO: 70 | 4350 |
| CII2184 | CDR | PRT | WO2014028776; SEQ ID NO: 68 | 4351 |
| CII2185 | CDR | PRT | WO2014028776; SEQ ID NO: 72 | 4352 |
| CII2186 | CDR | PRT | WO2014028776; SEQ ID NO: 73 | 4353 |
| CII2187 | CDR | PRT | WO2018140510; SEQ ID NO: 8 | 4354 |
| CII2188 | CDR | PRT | WO2918149510; SEQ ID NO: 10 | 4355 |
| CII2189 | CDR | PRT | US20180051086; SEQ ID NO: 9 | 4356 |
| CII2190 | CDR | PRT | US20180051086; SEQ ID NO: 10 | 4357 |
| CII2191 | CDR | PRT | US20180051086; SEQ ID NO: 12 | 4358 |
| CII2192 | CDR | PRT | WO2018140510; SEQ ID NO: 3 | 4359 |
| CII2193 | CDR | PRT | US20180051086; SEQ ID NO: 13 | 4360 |
| CII2194 | CDR | PRT | WO2018140510; SEQ ID NO: 5 | 4361 |
| CII2195 | CDR | PRT | WO2018140510; SEQ ID NO: 9 | 4362 |
| CII2196 | CDR | PRT | US20180051086; SEQ ID NO: 11 | 4363 |
| CII2197 | CDR | PRT | US20180051086; SEQ ID NO: 8 | 4364 |
| CII2198 | CDR | PRT | WO2018140510; SEQ ID NO: 4 | 4365 |
| CII2199 | FAb | PRT | WO2018162430; SEQ ID NO: 4 | 4366 |
| CII2200 | FAb | PRT | WO2018162430; SEQ ID NO: 2 | 4367 |
| CII2201 | FAb | PRT | WO2018162430; SEQ ID NO: 1 | 4368 |
| CII2202 | FAb | PRT | WO2018162430; SEQ ID NO: 3 | 4369 |
| CII2203 | Fc | PRT | WO2019028292; SEQ ID NO: 154 | 4370 |
| CII2204 | Fc | PRT | WO2019028292; SEQ ID NO: 155 | 4371 |
| CII2205 | Fc | PRT | WO2019028292; SEQ ID NO: 156 | 4372 |
| CII2206 | Fc | PRT | WO2019028292; SEQ ID NO: 148 | 4373 |
| CII2207 | Fc | PRT | WO2019028292; SEQ ID NO: 149 | 4374 |
| CII2208 | Fc | PRT | WO2019028292; SEQ ID NO: 150 | 4375 |
| CII2209 | Fc | PRT | WO2019028292; SEQ ID NO: 153 | 4376 |
| CII2210 | Fc | PRT | WO2019028292; SEQ ID NO: 146 | 4377 |
| CII2211 | Fc | PRT | WO2019028292; SEQ ID NO: 147 | 4378 |
| CII2212 | Fc | PRT | WO2019028292; SEQ ID NO: 151 | 4379 |
| CII2213 | Fc | PRT | WO2019028292; SEQ ID NO: 152 | 4380 |
| CII2214 | Fc | PRT | US20180221480; SEQ ID NO: 14 | 4381 |
| CII2215 | FR | PRT | WO2016201388; SEQ ID NO: 81 | 4382 |
| CII2216 | FR | PRT | WO2016201388; SEQ ID NO: 80 | 4383 |
| CII2217 | FR | PRT | W02016201389; SEQ ID NO: 239 | 4384 |
| CII2218 | FR | PRT | W02016201389; SEQ ID NO: 242 | 4385 |
| CII2219 | FR | PRT | W02016201389; SEQ ID NO: 237 | 4386 |
| CII2220 | FR | PRT | W02016201389; SEQ ID NO: 235 | 4387 |
| CII2221 | FR | PRT | W02016201389; SEQ ID NO: 236 | 4388 |
| CII2222 | FR | PRT | WO2016201388; SEQ ID NO: 79 | 4389 |
| CII2223 | FR | PRT | WO2016201388; SEQ ID NO: 234 | 4390 |
| CII2224 | FR | PRT | W02016201389; SEQ ID NO: 241 | 4391 |
| CII2225 | FR | PRT | W02016201389; SEQ ID NO: 232 | 4392 |
| CII2226 | FR | PRT | W02016201389; SEQ ID NO: 238 | 4393 |
| CII2227 | FR | PRT | W02016201389; SEQ ID NO: 231 | 4394 |
| CII2228 | FR | PRT | W02016201389; SEQ ID NO: 240 | 4395 |
| CII2229 | FR | PRT | W02016201389; SEQ ID NO: 234 | 4396 |
| CII2230 | FR | PRT | W02016201389; SEQ ID NO: 243 | 4397 |
| CII2231 | FR | PRT | W02016201389; SEQ ID NO: 233 | 4398 |
| CII2232 | FR | PRT | WO2016201388; SEQ ID NO: 107 | 4399 |
| CII2233 | FR | PRT | WO2016201388; SEQ ID NO: 106 | 4400 |
| CII2234 | FR | PRT | WO2016201388; SEQ ID NO: 105 | 4401 |
| CII2235 | FR | PRT | WO2016201388; SEQ ID NO: 240 | 4402 |
| CII2236 | FR | PRT | WO2016201388; SEQ ID NO: 96 | 4403 |
| CII2237 | FR | PRT | W02016201389; SEQ ID NO: 259 | 4404 |
| CII2238 | FR | PRT | WO2016201388; SEQ ID NO: 239 | 4405 |
| CII2239 | FR | PRT | WO2016201388; SEQ ID NO: 95 | 4406 |
| CII2240 | FR | PRT | W02016201389; SEQ ID NO: 267 | 4407 |
| CII2241 | FR | PRT | W02016201389; SEQ ID NO: 263 | 4408 |
| CII2242 | FR | PRT | WO2016201388; SEQ ID NO: 94 | 4409 |
| CII2243 | FR | PRT | WO2016201388; SEQ ID NO: 93 | 4410 |
| CII2244 | FR | PRT | WO2016201388; SEQ ID NO: 92 | 4411 |
| CII2245 | FR | PRT | W02016201389; SEQ ID NO: 258 | 4412 |
| CII2246 | FR | PRT | WO2016201388; SEQ ID NO: 238 | 4413 |
| CII2247 | FR | PRT | WO2019028283; SEQ ID NO: 164 | 4414 |
| CII2248 | FR | PRT | W02016201389; SEQ ID NO: 248 | 4415 |
| CII2249 | FR | PRT | W02016201389; SEQ ID NO: 250 | 4416 |
| CII2250 | FR | PRT | W02016201389; SEQ ID NO: 251 | 4417 |
| CII2251 | FR | PRT | WO2016201388; SEQ ID NO: 237 | 4418 |
| CII2252 | FR | PRT | WO2016201388; SEQ ID NO: 91 | 4419 |
| CII2253 | FR | PRT | WO2016201388; SEQ ID NO: 87 | 4420 |
| CII2254 | FR | PRT | WO2016201388; SEQ ID NO: 88 | 4421 |
| CII2255 | FR | PRT | W02016201389; SEQ ID NO: 255 | 4422 |
| CII2256 | FR | PRT | W02016201389; SEQ ID NO: 257 | 4423 |
| CII2257 | FR | PRT | W02016201389; SEQ ID NO: 249 | 4424 |
| CII2258 | FR | PRT | WO2016201388; SEQ ID NO: 90 | 4425 |
| CII2259 | FR | PRT | W02016201389; SEQ ID NO: 254 | 4426 |
| CII2260 | FR | PRT | W02016201389; SEQ ID NO: 252 | 4427 |
| CII2261 | FR | PRT | W02016201389; SEQ ID NO: 253 | 4428 |
| CII2262 | FR | PRT | W02016201389; SEQ ID NO: 256 | 4429 |
| CII2263 | FR | PRT | WO2016201388; SEQ ID NO: 89 | 4430 |
| CII2264 | FR | PRT | WO2016201388; SEQ ID NO: 99 | 4431 |
| CII2265 | FR | PRT | WO2016201388; SEQ ID NO: 104 | 4432 |
| CII2266 | FR | PRT | W02016201389; SEQ ID NO: 262 | 4433 |
| CII2267 | FR | PRT | WO2016201388; SEQ ID NO: 235 | 4434 |
| CII2268 | FR | PRT | W02016201389; SEQ ID NO: 266 | 4435 |
| CII2269 | FR | PRT | W02016201389; SEQ ID NO: 265 | 4436 |
| CII2270 | FR | PRT | WO2016201388; SEQ ID NO: 98 | 4437 |
| CII2271 | FR | PRT | WO2016201388; SEQ ID NO: 97 | 4438 |
| CII2272 | FR | PRT | W02016201389; SEQ ID NO: 264 | 4439 |
| CII2273 | FR | PRT | W02016201389; SEQ ID NO: 261 | 4440 |
| CII2274 | FR | PRT | W02016201389; SEQ ID NO: 260 | 4441 |
| CII2275 | FR | PRT | W02016201389; SEQ ID NO: 277 | 4442 |
| CII2276 | FR | PRT | W02016201389; SEQ ID NO: 273 | 4443 |
| CII2277 | FR | PRT | W02016201389; SEQ ID NO: 276 | 4444 |
| CII2278 | FR | PRT | W02016201389; SEQ ID NO: 274 | 4445 |
| CII2279 | FR | PRT | W02016201389; SEQ ID NO: 275 | 4446 |
| CII2280 | FR | PRT | W02016201389; SEQ ID NO: 278 | 4447 |
| CII2281 | FR | PRT | WO2016201388; SEQ ID NO: 82 | 4448 |
| CII2282 | FR | PRT | W02016201389; SEQ ID NO: 284 | 4449 |
| CII2283 | FR | PRT | WO2016201388; SEQ ID NO: 110 | 4450 |
| CII2284 | FR | PRT | W02016201389; SEQ ID NO: 282 | 4451 |
| CII2285 | FR | PRT | WO2016201388; SEQ ID NO: 109 | 4452 |
| CII2286 | FR | PRT | W02016201389; SEQ ID NO: 279 | 4453 |
| CII2287 | FR | PRT | W02016201389; SEQ ID NO: 283 | 4454 |
| CII2288 | FR | PRT | WO2016201388; SEQ ID NO: 111 | 4455 |
| CII2289 | FR | PRT | WO2016201388; SEQ ID NO: 108 | 4456 |
| CII2290 | FR | PRT | W02016201389; SEQ ID NO: 281 | 4457 |
| CII2291 | FR | PRT | W02016201389; SEQ ID NO: 280 | 4458 |
| CII2292 | FR | PRT | W02016201389; SEQ ID NO: 272 | 4459 |
| CII2293 | FR | PRT | W02016201389; SEQ ID NO: 270 | 4460 |
| CII2294 | FR | PRT | WO2016201388; SEQ ID NO: 86 | 4461 |
| CII2295 | FR | PRT | WO2016201388; SEQ ID NO: 84 | 4462 |
| CII2296 | FR | PRT | WO2016201388; SEQ ID NO: 100 | 4463 |
| CII2297 | FR | PRT | WO2016201388; SEQ ID NO: 102 | 4464 |
| CII2298 | FR | PRT | W02016201389; SEQ ID NO: 271 | 4465 |
| CII2299 | FR | PRT | W02016201389; SEQ ID NO: 268 | 4466 |
| CII2300 | FR | PRT | W02016201389; SEQ ID NO: 269 | 4467 |
| CII2301 | FR | PRT | WO2016201388; SEQ ID NO: 103 | 4468 |
| CII2302 | FR | PRT | WO2016201388; SEQ ID NO: 101 | 4469 |
| CII2303 | FR | PRT | W02016201389; SEQ ID NO: 247 | 4470 |
| CII2304 | FR | PRT | W02016201389; SEQ ID NO: 246 | 4471 |
| CII2305 | FR | PRT | W02016201389; SEQ ID NO: 244 | 4472 |
| CII2306 | FR | PRT | WO2016201388; SEQ ID NO: 85 | 4473 |
| CII2307 | FR | PRT | WO2016201388; SEQ ID NO: 83 | 4474 |
| CII2308 | FR | PRT | W02016201389; SEQ ID NO: 245 | 4475 |
| CII2309 | FR | PRT | WO2016201388; SEQ ID NO: 236 | 4476 |
| CII2310 | FR | PRT | WO2016201388; SEQ ID NO: 78 | 4477 |
| CII2311 | FR | PRT | WO2018213316; SEQ ID NO: 59 | 4478 |
| CII2312 | FR | PRT | WO2018213316; SEQ ID NO: 60 | 4479 |
| CII2313 | FR | PRT | WO2018213316; SEQ ID NO: 56 | 4480 |
| CII2314 | FR | PRT | WO2018213316; SEQ ID NO: 57 | 4481 |
| CII2315 | FR | PRT | WO2018213316; SEQ ID NO: 61 | 4482 |
| CII2316 | FR | PRT | WO2018213316; SEQ ID NO: 58 | 4483 |
| CII2317 | FR | PRT | WO2018213316; SEQ ID NO: 78 | 4484 |
| CII2318 | FR | PRT | WO2018213316; SEQ ID NO: 83 | 4485 |
| CII2319 | FR | PRT | WO2018213316; SEQ ID NO: 77 | 4486 |
| CII2320 | FR | PRT | WO2018213316; SEQ ID NO: 76 | 4487 |
| CII2321 | FR | PRT | WO2018213316; SEQ ID NO: 74 | 4488 |
| CII2322 | FR | PRT | WO2018213316; SEQ ID NO: 75 | 4489 |
| CII2323 | FR | PRT | WO2018213316; SEQ ID NO: 73 | 4490 |
| CII2324 | FR | PRT | WO2018213316; SEQ ID NO: 68 | 4491 |
| CII2325 | FR | PRT | WO2018213316; SEQ ID NO: 72 | 4492 |
| CII2326 | FR | PRT | WO2018213316; SEQ ID NO: 71 | 4493 |
| CII2327 | FR | PRT | WO2018213316; SEQ ID NO: 70 | 4494 |
| CII2328 | FR | PRT | WO2018213316; SEQ ID NO: 69 | 4495 |
| CII2329 | FR | PRT | WO2018213316; SEQ ID NO: 95 | 4496 |
| CII2330 | FR | PRT | WO2018213316; SEQ ID NO: 94 | 4497 |
| CII2331 | FR | PRT | WO2018213316; SEQ ID NO: 99 | 4498 |
| CII2332 | FR | PRT | WO2018213316; SEQ ID NO: 93 | 4499 |
| CII2333 | FR | PRT | WO2018213316; SEQ ID NO: 96 | 4500 |
| CII2334 | FR | PRT | WO2018213316; SEQ ID NO: 88 | 4501 |
| CII2335 | FR | PRT | WO2018213316; SEQ ID NO: 92 | 4502 |
| CII2336 | FR | PRT | WO2018213316; SEQ ID NO: 89 | 4503 |
| CII2337 | FR | PRT | WO2018213316; SEQ ID NO: 91 | 4504 |
| CII2338 | FR | PRT | WO2018213316; SEQ ID NO: 98 | 4505 |
| CII2339 | FR | PRT | WO2018213316; SEQ ID NO: 90 | 4506 |
| CII2340 | FR | PRT | WO2018213316; SEQ ID NO: 55 | 4507 |
| CII2341 | FR | PRT | WO2018213316; SEQ ID NO: 79 | 4508 |
| CII2342 | FR | PRT | WO2018213316; SEQ ID NO: 80 | 4509 |
| CII2343 | FR | PRT | WO2018213316; SEQ ID NO: 82 | 4510 |
| CII2344 | FR | PRT | WO2018213316; SEQ ID NO: 81 | 4511 |
| CII2345 | FR | PRT | WO2018213316; SEQ ID NO: 97 | 4512 |
| CII2346 | FR | PRT | WO2018213316; SEQ ID NO: 100 | 4513 |
| CII2347 | FR | PRT | WO2018213316; SEQ ID NO: 101 | 4514 |
| CII2348 | FR | PRT | WO2018213316; SEQ ID NO: 102 | 4515 |
| CII2349 | FR | PRT | WO2018213316; SEQ ID NO: 103 | 4516 |
| CII2350 | FR | PRT | WO2018213316; SEQ ID NO: 87 | 4517 |
| CII2351 | FR | PRT | WO2018213316; SEQ ID NO: 86 | 4518 |
| CII2352 | FR | PRT | WO2018213316; SEQ ID NO: 85 | 4519 |
| CII2353 | FR | PRT | WO2018213316; SEQ ID NO: 84 | 4520 |
| CII2354 | FR | PRT | WO2018213316; SEQ ID NO: 62 | 4521 |
| CII2355 | FR | PRT | WO2018213316; SEQ ID NO: 65 | 4522 |
| CII2356 | FR | PRT | WO2018213316; SEQ ID NO: 63 | 4523 |
| CII2357 | FR | PRT | WO2018213316; SEQ ID NO: 64 | 4524 |
| CII2358 | FR | PRT | WO2018213316; SEQ ID NO: 66 | 4525 |
| CII2359 | FR | PRT | WO2018213316; SEQ ID NO: 67 | 4526 |
| CII2360 | FR | PRT | WO2017040301; SEQ ID NO: 165 | 4527 |
| CII2361 | FR | PRT | WO2017040301; SEQ ID NO: 97 | 4528 |
| CII2362 | FR | PRT | WO2017040301; SEQ ID NO: 86 | 4529 |
| CII2363 | FR | PRT | WO2017040301; SEQ ID NO: 88 | 4530 |
| CII2364 | FR | PRT | WO2017040301; SEQ ID NO: 94 | 4531 |
| CII2365 | FR | PRT | WO2017040301; SEQ ID NO: 93 | 4532 |
| CII2366 | FR | PRT | WO2017040301; SEQ ID NO: 175 | 4533 |
| CII2367 | FR | PRT | WO2017040301; SEQ ID NO: 170 | 4534 |
| CII2368 | FR | PRT | WO2017040301; SEQ ID NO: 171 | 4535 |
| CII2369 | FR | PRT | WO2017040301; SEQ ID NO: 95 | 4536 |
| CII2370 | FR | PRT | WO2017040301; SEQ ID NO: 90 | 4537 |
| CII2371 | FR | PRT | WO2017040301; SEQ ID NO: 85 | 4538 |
| CII2372 | FR | PRT | WO2017040301; SEQ ID NO: 89 | 4539 |
| CII2373 | FR | PRT | WO2017040301; SEQ ID NO: 163 | 4540 |
| CII2374 | FR | PRT | WO2017040301; SEQ ID NO: 166 | 4541 |
| CII2375 | FR | PRT | WO2017040301; SEQ ID NO: 168 | 4542 |
| CII2376 | FR | PRT | WO2017040301; SEQ ID NO: 173 | 4543 |
| CII2377 | FR | PRT | WO2017040301; SEQ ID NO: 169 | 4544 |
| CII2378 | FR | PRT | WO2017040301; SEQ ID NO: 167 | 4545 |
| CII2379 | FR | PRT | WO2017040301; SEQ ID NO: 144 | 4546 |
| CII2380 | FR | PRT | WO2017040301; SEQ ID NO: 149 | 4547 |
| CII2381 | FR | PRT | WO2017040301; SEQ ID NO: 139 | 4548 |
| CII2382 | FR | PRT | WO2017040301; SEQ ID NO: 131 | 4549 |
| CII2383 | FR | PRT | WO2017040301; SEQ ID NO: 124 | 4550 |
| CII2384 | FR | PRT | WO2017040301; SEQ ID NO: 128 | 4551 |
| CII2385 | FR | PRT | WO2017040301; SEQ ID NO: 129 | 4552 |
| CII2386 | FR | PRT | WO2017040301; SEQ ID NO: 130 | 4553 |
| CII2387 | FR | PRT | WO2017040301; SEQ ID NO: 132 | 4554 |
| CII2388 | FR | PRT | WO2017040301; SEQ ID NO: 133 | 4555 |
| CII2389 | FR | PRT | WO2017040301; SEQ ID NO: 134 | 4556 |
| CII2390 | FR | PRT | WO2017040301; SEQ ID NO: 135 | 4557 |
| CII2391 | FR | PRT | WO2017040301; SEQ ID NO: 136 | 4558 |
| CII2392 | FR | PRT | WO2017040301; SEQ ID NO: 126 | 4559 |
| CII2393 | FR | PRT | WO2017040301; SEQ ID NO: 125 | 4560 |
| CII2394 | FR | PRT | WO2017040301; SEQ ID NO: 127 | 4561 |
| CII2395 | FR | PRT | WO2017040301; SEQ ID NO: 114 | 4562 |
| CII2396 | FR | PRT | WO2017040301; SEQ ID NO: 104 | 4563 |
| CII2397 | FR | PRT | WO2017040301; SEQ ID NO: 123 | 4564 |
| CII2398 | FR | PRT | WO2017040301; SEQ ID NO: 119 | 4565 |
| CII2399 | FR | PRT | WO2017040301; SEQ ID NO: 112 | 4566 |
| CII2400 | FR | PRT | WO2017040301; SEQ ID NO: 174 | 4567 |
| CII2401 | FR | PRT | WO2017040301; SEQ ID NO: 111 | 4568 |
| CII2402 | FR | PRT | WO2017040301; SEQ ID NO: 115 | 4569 |
| CII2403 | FR | PRT | WO2017040301; SEQ ID NO: 121 | 4570 |
| CII2404 | FR | PRT | WO2017040301; SEQ ID NO: 116 | 4571 |
| CII2405 | FR | PRT | WO2017040301; SEQ ID NO: 113 | 4572 |
| CII2406 | FR | PRT | WO2017040301; SEQ ID NO: 118 | 4573 |
| CII2407 | FR | PRT | WO2017040301; SEQ ID NO: 120 | 4574 |
| CII2408 | FR | PRT | WO2017040301; SEQ ID NO: 117 | 4575 |
| CII2409 | FR | PRT | WO2017040301; SEQ ID NO: 122 | 4576 |
| CII2410 | FR | PRT | WO2017040301; SEQ ID NO: 92 | 4577 |
| CII2411 | FR | PRT | WO2017040301; SEQ ID NO: 145 | 4578 |
| CII2412 | FR | PRT | WO2017040301; SEQ ID NO: 91 | 4579 |
| CII2413 | FR | PRT | WO2017040301; SEQ ID NO: 164 | 4580 |
| CII2414 | FR | PRT | WO2017040301; SEQ ID NO: 146 | 4581 |
| CII2415 | FR | PRT | WO2017040301; SEQ ID NO: 141 | 4582 |
| CII2416 | FR | PRT | WO2017040301; SEQ ID NO: 143 | 4583 |
| CII2417 | FR | PRT | WO2017040301; SEQ ID NO: 138 | 4584 |
| CII2418 | FR | PRT | WO2017040301; SEQ ID NO: 148 | 4585 |
| CII2419 | FR | PRT | WO2017040301; SEQ ID NO: 137 | 4586 |
| CII2420 | FR | PRT | WO2017040301; SEQ ID NO: 140 | 4587 |
| CII2421 | FR | PRT | WO2017040301; SEQ ID NO: 142 | 4588 |
| CII2422 | FR | PRT | WO2017040301; SEQ ID NO: 147 | 4589 |
| CII2423 | FR | PRT | WO2017040301; SEQ ID NO: 162 | 4590 |
| CII2424 | FR | PRT | WO2017040301; SEQ ID NO: 172 | 4591 |
| CII2425 | FR | PRT | WO2017040301; SEQ ID NO: 87 | 4592 |
| CII2426 | FR | PRT | WO2017040301; SEQ ID NO: 178 | 4593 |
| CII2427 | FR | PRT | WO2017040301; SEQ ID NO: 183 | 4594 |
| CII2428 | FR | PRT | WO2017040301; SEQ ID NO: 184 | 4595 |
| CII2429 | FR | PRT | WO2017040301; SEQ ID NO: 188 | 4596 |
| CII2430 | FR | PRT | WO2017040301; SEQ ID NO: 180 | 4597 |
| CII2431 | FR | PRT | WO2017040301; SEQ ID NO: 176 | 4598 |
| CII2432 | FR | PRT | WO2017040301; SEQ ID NO: 177 | 4599 |
| CII2433 | FR | PRT | WO2017040301; SEQ ID NO: 179 | 4600 |
| CII2434 | FR | PRT | WO2017040301; SEQ ID NO: 181 | 4601 |
| CII2435 | FR | PRT | WO2017040301; SEQ ID NO: 185 | 4602 |
| CII2436 | FR | PRT | WO2017040301; SEQ ID NO: 186 | 4603 |
| CII2437 | FR | PRT | WO2017040301; SEQ ID NO: 182 | 4604 |
| CII2438 | FR | PRT | WO2017040301; SEQ ID NO: 187 | 4605 |
| CII2439 | FR | PRT | WO2017040301; SEQ ID NO: 151 | 4606 |
| CII2440 | FR | PRT | WO2017040301; SEQ ID NO: 156 | 4607 |
| CII2441 | FR | PRT | WO2017040301; SEQ ID NO: 154 | 4608 |
| CII2442 | FR | PRT | WO2017040301; SEQ ID NO: 159 | 4609 |
| CII2443 | FR | PRT | WO2017040301; SEQ ID NO: 150 | 4610 |
| CII2444 | FR | PRT | WO2017040301; SEQ ID NO: 153 | 4611 |
| CII2445 | FR | PRT | WO2017040301; SEQ ID NO: 155 | 4612 |
| CII2446 | FR | PRT | WO2017040301; SEQ ID NO: 160 | 4613 |
| CII2447 | FR | PRT | WO2017040301; SEQ ID NO: 152 | 4614 |
| CII2448 | FR | PRT | WO2017040301; SEQ ID NO: 157 | 4615 |
| CII2449 | FR | PRT | WO2017040301; SEQ ID NO: 161 | 4616 |
| CII2450 | FR | PRT | WO2017040301; SEQ ID NO: 158 | 4617 |
| CII2451 | FR | PRT | WO2017040301; SEQ ID NO: 109 | 4618 |
| CII2452 | FR | PRT | WO2017040301; SEQ ID NO: 106 | 4619 |
| CII2453 | FR | PRT | WO2017040301; SEQ ID NO: 102 | 4620 |
| CII2454 | FR | PRT | WO2017040301; SEQ ID NO: 103 | 4621 |
| CII2455 | FR | PRT | WO2017040301; SEQ ID NO: 108 | 4622 |
| CII2456 | FR | PRT | WO2017040301; SEQ ID NO: 98 | 4623 |
| CII2457 | FR | PRT | WO2017040301; SEQ ID NO: 99 | 4624 |
| CII2458 | FR | PRT | WO2017040301; SEQ ID NO: 107 | 4625 |
| CII2459 | FR | PRT | WO2017040301; SEQ ID NO: 101 | 4626 |
| CII2460 | FR | PRT | WO2017040301; SEQ ID NO: 100 | 4627 |
| CII2461 | FR | PRT | WO2017040301; SEQ ID NO: 105 | 4628 |
| CII2462 | FR | PRT | WO2017040301; SEQ ID NO: 110 | 4629 |
| CII2463 | FR | PRT | WO2017040301; SEQ ID NO: 96 | 4630 |
| CII2464 | FR | PRT | WO2017075432; SEQ ID NO: 171 | 4631 |
| CII2465 | FR | PRT | WO2017075432; SEQ ID NO: 33 | 4632 |
| CII2466 | FR | PRT | WO2017075432; SEQ ID NO: 30 | 4633 |
| CII2467 | FR | PRT | WO2017075432; SEQ ID NO: 185 | 4634 |
| CII2468 | FR | PRT | WO2017075432; SEQ ID NO: 54 | 4635 |
| CII2469 | FR | PRT | WO2017075432; SEQ ID NO: 184 | 4636 |
| CII2470 | FR | PRT | WO2017075432; SEQ ID NO: 31 | 4637 |
| CII2471 | FR | PRT | WO2017075432; SEQ ID NO: 56 | 4638 |
| CII2472 | FR | PRT | WO2017075432; SEQ ID NO: 47 | 4639 |
| CII2473 | FR | PRT | WO2017075432; SEQ ID NO: 190 | 4640 |
| CII2474 | FR | PRT | WO2017075432; SEQ ID NO: 191 | 4641 |
| CII2475 | FR | PRT | WO2017075432; SEQ ID NO: 45 | 4642 |
| CII2476 | FR | PRT | WO2017075432; SEQ ID NO: 44 | 4643 |
| CII2477 | FR | PRT | WO2017075432; SEQ ID NO: 46 | 4644 |
| CII2478 | FR | PRT | WO2017075432; SEQ ID NO: 42 | 4645 |
| CII2479 | FR | PRT | WO2017075432; SEQ ID NO: 189 | 4646 |
| CII2480 | FR | PRT | WO2017075432; SEQ ID NO: 55 | 4647 |
| CII2481 | FR | PRT | WO2017075432; SEQ ID NO: 38 | 4648 |
| CII2482 | FR | PRT | WO2017075432; SEQ ID NO: 188 | 4649 |
| CII2483 | FR | PRT | WO2017075432; SEQ ID NO: 43 | 4650 |
| CII2484 | FR | PRT | WO2017075432; SEQ ID NO: 41 | 4651 |
| CII2485 | FR | PRT | WO2017075432; SEQ ID NO: 40 | 4652 |
| CII2486 | FR | PRT | WO2017075432; SEQ ID NO: 39 | 4653 |
| CII2487 | FR | PRT | WO2017075432; SEQ ID NO: 32 | 4654 |
| CII2488 | FR | PRT | WO2017075432; SEQ ID NO: 34 | 4655 |
| CII2489 | FR | PRT | WO2017075432; SEQ ID NO: 57 | 4656 |
| CII2490 | FR | PRT | WO2017075432; SEQ ID NO: 49 | 4657 |
| CII2491 | FR | PRT | WO2017075432; SEQ ID NO: 48 | 4658 |
| CII2492 | FR | PRT | WO2017075432; SEQ ID NO: 50 | 4659 |
| CII2493 | FR | PRT | WO2017075432; SEQ ID NO: 59 | 4660 |
| CII2494 | FR | PRT | WO2017075432; SEQ ID NO: 196 | 4661 |
| CII2495 | FR | PRT | WO2017075432; SEQ ID NO: 58 | 4662 |
| CII2496 | FR | PRT | WO2017075432; SEQ ID NO: 60 | 4663 |
| CII2497 | FR | PRT | WO2017075432; SEQ ID NO: 37 | 4664 |
| CII2498 | FR | PRT | WO2017075432; SEQ ID NO: 36 | 4665 |
| CII2499 | FR | PRT | WO2017075432; SEQ ID NO: 35 | 4666 |
| CII2500 | FR | PRT | WO2017075432; SEQ ID NO: 52 | 4667 |
| CII2501 | FR | PRT | WO2017075432; SEQ ID NO: 53 | 4668 |
| CII2502 | FR | PRT | WO2017075432; SEQ ID NO: 192 | 4669 |
| CII2503 | FR | PRT | WO2017075432; SEQ ID NO: 193 | 4670 |
| CII2504 | FR | PRT | WO2017075432; SEQ ID NO: 51 | 4671 |
| CII2505 | FR | PRT | WO2017075432; SEQ ID NO: 186 | 4672 |
| CII2506 | FR | PRT | WO2017075432; SEQ ID NO: 187 | 4673 |
| CII2507 | FR | PRT | WO2017075432; SEQ ID NO: 194 | 4674 |
| CII2508 | FR | PRT | WO2017075432; SEQ ID NO: 195 | 4675 |
| CII2509 | FR | PRT | WO2018107058; SEQ ID NO: 22 | 4676 |
| CII2510 | FR | PRT | WO2018107058; SEQ ID NO: 26 | 4677 |
| CII2511 | FR | PRT | WO2018107058; SEQ ID NO: 18 | 4678 |
| CII2512 | FR | PRT | WO2018107058; SEQ ID NO: 25 | 4679 |
| CII2513 | FR | PRT | WO2018107058; SEQ ID NO: 33 | 4680 |
| CII2514 | FR | PRT | WO2018107058; SEQ ID NO: 24 | 4681 |
| CII2515 | FR | PRT | WO2018107058; SEQ ID NO: 32 | 4682 |
| CII2516 | FR | PRT | WO2018107058; SEQ ID NO: 30 | 4683 |
| CII2517 | FR | PRT | WO2018107058; SEQ ID NO: 21 | 4684 |
| CII2518 | FR | PRT | WO2018107058; SEQ ID NO: 29 | 4685 |
| CII2519 | FR | PRT | WO2018107058; SEQ ID NO: 27 | 4686 |
| CII2520 | FR | PRT | WO2018107058; SEQ ID NO: 19 | 4687 |
| CII2521 | FR | PRT | WO2018107058; SEQ ID NO: 23 | 4688 |
| CII2522 | FR | PRT | WO2018107058; SEQ ID NO: 31 | 4689 |
| CII2523 | FR | PRT | WO2018107058; SEQ ID NO: 28 | 4690 |
| CII2524 | FR | PRT | WO2018107058; SEQ ID NO: 20 | 4691 |
| CII2525 | FR | PRT | WO2016164637; SEQ ID NO: 268 | 4692 |
| CII2526 | FR | PRT | US20190085084; SEQ ID NO: 258 | 4693 |
| CII2527 | FR | PRT | WO2016164637; SEQ ID NO: 258 | 4694 |
| CII2528 | FR | PRT | US20190085084; SEQ ID NO: 263 | 4695 |
| CII2529 | FR | PRT | WO2016164637; SEQ ID NO: 263 | 4696 |
| CII2530 | FR | PRT | US20190085084; SEQ ID NO: 259 | 4697 |
| CII2531 | FR | PRT | WO2016164637; SEQ ID NO: 259 | 4698 |
| CII2532 | FR | PRT | US20190085084; SEQ ID NO: 267 | 4699 |
| CII2533 | FR | PRT | WO2016164637; SEQ ID NO: 267 | 4700 |
| CII2534 | FR | PRT | US20190085084; SEQ ID NO: 261 | 4701 |
| CII2535 | FR | PRT | WO2016164637; SEQ ID NO: 261 | 4702 |
| CII2536 | FR | PRT | US20190085084; SEQ ID NO: 264 | 4703 |
| CII2537 | FR | PRT | WO2016164637; SEQ ID NO: 264 | 4704 |
| CII2538 | FR | PRT | US20190085084; SEQ ID NO: 262 | 4705 |
| CII2539 | FR | PRT | WO2016164637; SEQ ID NO: 262 | 4706 |
| CII2540 | FR | PRT | US20190085084; SEQ ID NO: 257 | 4707 |
| CII2541 | FR | PRT | WO2016164637; SEQ ID NO: 257 | 4708 |
| CII2542 | FR | PRT | US20190085084; SEQ ID NO: 266 | 4709 |
| CII2543 | FR | PRT | WO2016164637; SEQ ID NO: 266 | 4710 |
| CII2544 | FR | PRT | US20190085084; SEQ ID NO: 260 | 4711 |
| CII2545 | FR | PRT | WO2016164637; SEQ ID NO: 260 | 4712 |
| CII2546 | FR | PRT | US20190085084; SEQ ID NO: 265 | 4713 |
| CII2547 | FR | PRT | WO2016164637; SEQ ID NO: 265 | 4714 |
| CII2548 | FR | PRT | US20190085084; SEQ ID NO: 289 | 4715 |
| CII2549 | FR | PRT | WO2016164637; SEQ ID NO: 289 | 4716 |
| CII2550 | FR | PRT | US20190085084; SEQ ID NO: 288 | 4717 |
| CII2551 | FR | PRT | WO2016164637; SEQ ID NO: 288 | 4718 |
| CII2552 | FR | PRT | US20190085084; SEQ ID NO: 287 | 4719 |
| CII2553 | FR | PRT | WO2016164637; SEQ ID NO: 287 | 4720 |
| CII2554 | FR | PRT | US20190085084; SEQ ID NO: 295 | 4721 |
| CII2555 | FR | PRT | WO2016164637; SEQ ID NO: 295 | 4722 |
| CII2556 | FR | PRT | US20190085084; SEQ ID NO: 283 | 4723 |
| CII2557 | FR | PRT | WO2016164637; SEQ ID NO: 283 | 4724 |
| CII2558 | FR | PRT | US20190085084; SEQ ID NO: 31 | 4725 |
| CII2559 | FR | PRT | US20190085084; SEQ ID NO: 281 | 4726 |
| CII2560 | FR | PRT | WO2016164637; SEQ ID NO: 281 | 4727 |
| CII2561 | FR | PRT | US20190085084; SEQ ID NO: 279 | 4728 |
| CII2562 | FR | PRT | WO2016164637; SEQ ID NO: 279 | 4729 |
| CII2563 | FR | PRT | US20190085084; SEQ ID NO: 276 | 4730 |
| CII2564 | FR | PRT | WO2016164637; SEQ ID NO: 276 | 4731 |
| CII2565 | FR | PRT | US20190085084; SEQ ID NO: 280 | 4732 |
| CII2566 | FR | PRT | WO2016164637; SEQ ID NO: 280 | 4733 |
| CII2567 | FR | PRT | US20190085084; SEQ ID NO: 282 | 4734 |
| CII2568 | FR | PRT | WO2016164637; SEQ ID NO: 282 | 4735 |
| CII2569 | FR | PRT | US20190085084; SEQ ID NO: 277 | 4736 |
| CII2570 | FR | PRT | WO2016164637; SEQ ID NO: 277 | 4737 |
| CII2571 | FR | PRT | US20190085084; SEQ ID NO: 278 | 4738 |
| CII2572 | FR | PRT | WO2016164637; SEQ ID NO: 278 | 4739 |
| CII2573 | FR | PRT | US20190085084; SEQ ID NO: 294 | 4740 |
| CII2574 | FR | PRT | WO2016164637; SEQ ID NO: 294 | 4741 |
| CII2575 | FR | PRT | US20190085084; SEQ ID NO: 291 | 4742 |
| CII2576 | FR | PRT | WO2016164637; SEQ ID NO: 291 | 4743 |
| CII2577 | FR | PRT | US20190085084; SEQ ID NO: 292 | 4744 |
| CII2578 | FR | PRT | WO2016164637; SEQ ID NO: 292 | 4745 |
| CII2579 | FR | PRT | US20190085084; SEQ ID NO: 290 | 4746 |
| CII2580 | FR | PRT | WO2016164637; SEQ ID NO: 290 | 4747 |
| CII2581 | FR | PRT | US20190085084; SEQ ID NO: 284 | 4748 |
| CII2582 | FR | PRT | WO2016164637; SEQ ID NO: 284 | 4749 |
| CII2583 | FR | PRT | US20190085084; SEQ ID NO: 285 | 4750 |
| CII2584 | FR | PRT | WO2016164637; SEQ ID NO: 285 | 4751 |
| CII2585 | FR | PRT | US20190085084; SEQ ID NO: 310 | 4752 |
| CII2586 | FR | PRT | WO2016164637; SEQ ID NO: 310 | 4753 |
| CII2587 | FR | PRT | US20190085084; SEQ ID NO: 307 | 4754 |
| CII2588 | FR | PRT | WO2016164637; SEQ ID NO: 307 | 4755 |
| CII2589 | FR | PRT | US20190085084; SEQ ID NO: 306 | 4756 |
| CII2590 | FR | PRT | WO2016164637; SEQ ID NO: 306 | 4757 |
| CII2591 | FR | PRT | US20190085084; SEQ ID NO: 308 | 4758 |
| CII2592 | FR | PRT | WO2016164637; SEQ ID NO: 308 | 4759 |
| CII2593 | FR | PRT | US20190085084; SEQ ID NO: 309 | 4760 |
| CII2594 | FR | PRT | WO2016164637; SEQ ID NO: 309 | 4761 |
| CII2595 | FR | PRT | US20199085084; SEQ ID NO: 305 | 4762 |
| CII2596 | FR | PRT | WO2016164637; SEQ ID NO: 305 | 4763 |
| CII2597 | FR | PRT | US20190085084; SEQ ID NO: 302 | 4764 |
| CII2598 | FR | PRT | WO2016164637; SEQ ID NO: 302 | 4765 |
| CII2599 | FR | PRT | US20190085084; SEQ ID NO: 304 | 4766 |
| CII2600 | FR | PRT | WO2016164637; SEQ ID NO: 304 | 4767 |
| CII2601 | FR | PRT | US20190085084; SEQ ID NO: 303 | 4768 |
| CII2602 | FR | PRT | WO2016164637; SEQ ID NO: 303 | 4769 |
| CII2603 | FR | PRT | US29190085084; SEQ ID NO: 315 | 4770 |
| CII2604 | FR | PRT | WO2016164637; SEQ ID NO: 315 | 4771 |
| CII2605 | FR | PRT | US20190085084; SEQ ID NO: 312 | 4772 |
| CII2606 | FR | PRT | WO2016164637; SEQ ID NO: 312 | 4773 |
| CII2607 | FR | PRT | US20190085084; SEQ ID NO: 313 | 4774 |
| CII2608 | FR | PRT | WO2016164637; SEQ ID NO: 313 | 4775 |
| CII2609 | FR | PRT | US29190085084; SEQ ID NO: 311 | 4776 |
| CII2610 | FR | PRT | WO2016164637; SEQ ID NO: 311 | 4777 |
| CII2611 | FR | PRT | US20190085084; SEQ ID NO: 314 | 4778 |
| CII2612 | FR | PRT | WO2016164637; SEQ ID NO: 314 | 4779 |
| CII2613 | FR | PRT | US20190085084; SEQ ID NO: 300 | 4780 |
| CII2614 | FR | PRT | WO2016164637; SEQ ID NO: 300 | 4781 |
| CII2615 | FR | PRT | US20190085084; SEQ ID NO: 299 | 4782 |
| CII2616 | FR | PRT | WO2016164637; SEQ ID NO: 299 | 4783 |
| CII2617 | FR | PRT | US20190085084; SEQ ID NO: 297 | 4784 |
| CII2618 | FR | PRT | WO2016164637; SEQ ID NO: 297 | 4785 |
| CII2619 | FR | PRT | US20190085084; SEQ ID NO: 298 | 4786 |
| CII2620 | FR | PRT | WO2016164637; SEQ ID NO: 298 | 4787 |
| CII2621 | FR | PRT | US20190085084; SEQ ID NO: 296 | 4788 |
| CII2622 | FR | PRT | WO2016164637; SEQ ID NO: 296 | 4789 |
| CII2623 | FR | PRT | US20190085084; SEQ ID NO: 301 | 4790 |
| CII2624 | FR | PRT | WO2016164637; SEQ ID NO: 301 | 4791 |
| CII2625 | FR | PRT | WO2016164637; SEQ ID NO: 272 | 4792 |
| CII2626 | FR | PRT | US20190085084; SEQ ID NO: 271 | 4793 |
| CII2627 | FR | PRT | WO2016164637; SEQ ID NO: 271 | 4794 |
| CII2628 | FR | PRT | US20190085084; SEQ ID NO: 275 | 4795 |
| CII2629 | FR | PRT | WO2016164637; SEQ ID NO: 275 | 4796 |
| CII2630 | FR | PRT | US20190085084; SEQ ID NO: 274 | 4797 |
| CII2631 | FR | PRT | WO2016164637; SEQ ID NO: 274 | 4798 |
| CII2632 | FR | PRT | US20190085084; SEQ ID NO: 270 | 4799 |
| CII2633 | FR | PRT | WO2016164637; SEQ ID NO: 270 | 4800 |
| CII2634 | FR | PRT | US20190085084; SEQ ID NO: 269 | 4801 |
| CII2635 | FR | PRT | WO2016164637; SEQ ID NO: 269 | 4802 |
| CII2636 | FR | PRT | US20190085084; SEQ ID NO: 273 | 4803 |
| CII2637 | FR | PRT | WO2016164637; SEQ ID NO: 273 | 4804 |
| CII2638 | FR | PRT | WO2017062672; SEQ ID NO: 723 | 4805 |
| CII2639 | FR | PRT | WO2017062672; SEQ ID NO: 775 | 4806 |
| CII2640 | FR | PRT | WO2017062672; SEQ ID NO: 114 | 4807 |
| CII2641 | FR | PRT | WO2017062672; SEQ ID NO: 774 | 4808 |
| CII2642 | FR | PRT | WO2017062672; SEQ ID NO: 592 | 4809 |
| CII2643 | FR | PRT | WO2017062672; SEQ ID NO: 713 | 4810 |
| CII2644 | FR | PRT | WO2017062672; SEQ ID NO: 711 | 4811 |
| CII2645 | FR | PRT | WO2017062672; SEQ ID NO: 714 | 4812 |
| CII2646 | FR | PRT | WO2017062672; SEQ ID NO: 118 | 4813 |
| CII2647 | FR | PRT | WO2017062672; SEQ ID NO: 103 | 4814 |
| CII2648 | FR | PRT | WO2017062672; SEQ ID NO: 722 | 4815 |
| CII2649 | FR | PRT | WO2017062672; SEQ ID NO: 208 | 4816 |
| CII2650 | FR | PRT | WO2017062672; SEQ ID NO: 194 | 4817 |
| CII2651 | FR | PRT | WO2017062672; SEQ ID NO: 195 | 4818 |
| CII2652 | FR | PRT | WO2017062672; SEQ ID NO: 204 | 4819 |
| CII2653 | FR | PRT | WO2017062672; SEQ ID NO: 206 | 4820 |
| CII2654 | FR | PRT | WO2017062672; SEQ ID NO: 199 | 4821 |
| CII2655 | FR | PRT | WO2017062672; SEQ ID NO: 197 | 4822 |
| CII2656 | FR | PRT | WO2017062672; SEQ ID NO: 192 | 4823 |
| CII2657 | FR | PRT | WO2017062672; SEQ ID NO: 188 | 4824 |
| CII2658 | FR | PRT | WO2017062672; SEQ ID NO: 116 | 4825 |
| CII2659 | FR | PRT | WO2017062672; SEQ ID NO: 712 | 4826 |
| CII2660 | FR | PRT | WO2017062672; SEQ ID NO: 110 | 4827 |
| CII2661 | FR | PRT | WO2017062672; SEQ ID NO: 773 | 4828 |
| CII2662 | FR | PRT | WO2017062672; SEQ ID NO: 111 | 4829 |
| CII2663 | FR | PRT | WO2017062672; SEQ ID NO: 115 | 4830 |
| CII2664 | FR | PRT | WO2017062672; SEQ ID NO: 107 | 4831 |
| CII2665 | FR | PRT | WO2017062672; SEQ ID NO: 105 | 4832 |
| CII2666 | FR | PRT | WO2017062672; SEQ ID NO: 203 | 4833 |
| CII2667 | FR | PRT | WO2017062672; SEQ ID NO: 193 | 4834 |
| CII2668 | FR | PRT | WO2017062672; SEQ ID NO: 196 | 4835 |
| CII2669 | FR | PRT | WO2017062672; SEQ ID NO: 200 | 4836 |
| CII2670 | FR | PRT | WO2017062672; SEQ ID NO: 205 | 4837 |
| CII2671 | FR | PRT | WO2017062672; SEQ ID NO: 599 | 4838 |
| CII2672 | FR | PRT | WO2017062672; SEQ ID NO: 804 | 4839 |
| CII2673 | FR | PRT | WO2017062672; SEQ ID NO: 198 | 4840 |
| CII2674 | FR | PRT | WO2017062672; SEQ ID NO: 191 | 4841 |
| CII2675 | FR | PRT | WO2017062672; SEQ ID NO: 159 | 4842 |
| CII2676 | FR | PRT | WO2017062672; SEQ ID NO: 165 | 4843 |
| CII2677 | FR | PRT | WO2017062672; SEQ ID NO: 156 | 4844 |
| CII2678 | FR | PRT | WO2017062672; SEQ ID NO: 168 | 4845 |
| CII2679 | FR | PRT | WO2017062672; SEQ ID NO: 157 | 4846 |
| CII2680 | FR | PRT | WO2017062672; SEQ ID NO: 719 | 4847 |
| CII2681 | FR | PRT | WO2017062672; SEQ ID NO: 170 | 4848 |
| CII2682 | FR | PRT | WO2017062672; SEQ ID NO: 160 | 4849 |
| CII2683 | FR | PRT | WO2017062672; SEQ ID NO: 172 | 4850 |
| CII2684 | FR | PRT | WO2017062672; SEQ ID NO: 164 | 4851 |
| CII2685 | FR | PRT | WO2017062672; SEQ ID NO: 785 | 4852 |
| CII2686 | FR | PRT | WO2017062672; SEQ ID NO: 149 | 4853 |
| CII2687 | FR | PRT | WO2017062672; SEQ ID NO: 151 | 4854 |
| CII2688 | FR | PRT | WO2017062672; SEQ ID NO: 148 | 4855 |
| CII2689 | FR | PRT | WO2017062672; SEQ ID NO: 152 | 4856 |
| CII2690 | FR | PRT | WO2017062672; SEQ ID NO: 155 | 4857 |
| CII2691 | FR | PRT | WO2017062672; SEQ ID NO: 154 | 4858 |
| CII2692 | FR | PRT | WO2017062672; SEQ ID NO: 150 | 4859 |
| CII2693 | FR | PRT | WO2017062672; SEQ ID NO: 153 | 4860 |
| CII2694 | FR | PRT | WO2017062672; SEQ ID NO: 202 | 4861 |
| CII2695 | FR | PRT | WO2017062672; SEQ ID NO: 784 | 4862 |
| CII2696 | FR | PRT | WO2017062672; SEQ ID NO: 201 | 4863 |
| CII2697 | FR | PRT | WO2017062672; SEQ ID NO: 782 | 4864 |
| CII2698 | FR | PRT | WO2017062672; SEQ ID NO: 134 | 4865 |
| CII2699 | FR | PRT | WO2017062672; SEQ ID NO: 136 | 4866 |
| CII2700 | FR | PRT | WO2017062672; SEQ ID NO: 132 | 4867 |
| CII2701 | FR | PRT | WO2017062672; SEQ ID NO: 138 | 4868 |
| CII2702 | FR | PRT | WO2017062672; SEQ ID NO: 848 | 4869 |
| CII2703 | FR | PRT | WO2017062672; SEQ ID NO: 143 | 4870 |
| CII2704 | FR | PRT | WO2017062672; SEQ ID NO: 135 | 4871 |
| CII2705 | FR | PRT | WO2017062672; SEQ ID NO: 137 | 4872 |
| CII2706 | FR | PRT | WO2017062672; SEQ ID NO: 140 | 4873 |
| CII2707 | FR | PRT | WO2017062672; SEQ ID NO: 144 | 4874 |
| CII2708 | FR | PRT | WO2017062672; SEQ ID NO: 781 | 4875 |
| CII2709 | FR | PRT | WO2017062672; SEQ ID NO: 715 | 4876 |
| CII2710 | FR | PRT | WO2017062672; SEQ ID NO: 594 | 4877 |
| CII2711 | FR | PRT | WO2017062672; SEQ ID NO: 717 | 4878 |
| CII2712 | FR | PRT | WO2017062672; SEQ ID NO: 718 | 4879 |
| CII2713 | FR | PRT | WO2017062672; SEQ ID NO: 133 | 4880 |
| CII2714 | FR | PRT | WO2017062672; SEQ ID NO: 595 | 4881 |
| CII2715 | FR | PRT | WO2017062672; SEQ ID NO: 139 | 4882 |
| CII2716 | FR | PRT | WO2017062672; SEQ ID NO: 131 | 4883 |
| CII2717 | FR | PRT | WO2017062672; SEQ ID NO: 147 | 4884 |
| CII2718 | FR | PRT | WO2017062672; SEQ ID NO: 716 | 4885 |
| CII2719 | FR | PRT | WO2017062672; SEQ ID NO: 145 | 4886 |
| CII2720 | FR | PRT | WO2017062672; SEQ ID NO: 182 | 4887 |
| CII2721 | FR | PRT | WO2017062672; SEQ ID NO: 142 | 4888 |
| CII2722 | FR | PRT | WO2017062672; SEQ ID NO: 141 | 4889 |
| CII2723 | FR | PRT | WO2017062672; SEQ ID NO: 130 | 4890 |
| CII2724 | FR | PRT | WO2017062672; SEQ ID NO: 783 | 4891 |
| CII2725 | FR | PRT | WO2017062672; SEQ ID NO: 596 | 4892 |
| CII2726 | FR | PRT | WO2017062672; SEQ ID NO: 146 | 4893 |
| CII2727 | FR | PRT | WO2017062672; SEQ ID NO: 799 | 4894 |
| CII2728 | FR | PRT | WO2017062672; SEQ ID NO: 776 | 4895 |
| CII2729 | FR | PRT | WO2017062672; SEQ ID NO: 112 | 4896 |
| CII2730 | FR | PRT | WO2017062672; SEQ ID NO: 166 | 4897 |
| CII2731 | FR | PRT | WO2017062672; SEQ ID NO: 772 | 4898 |
| CII2732 | FR | PRT | WO2017062672; SEQ ID NO: 117 | 4899 |
| CII2733 | FR | PRT | WO2017062672; SEQ ID NO: 109 | 4900 |
| CII2734 | FR | PRT | WO2017062672; SEQ ID NO: 800 | 4901 |
| CII2735 | FR | PRT | WO2017062672; SEQ ID NO: 207 | 4902 |
| CII2736 | FR | PRT | WO2017062672; SEQ ID NO: 802 | 4903 |
| CII2737 | FR | PRT | WO2017062672; SEQ ID NO: 190 | 4904 |
| CII2738 | FR | PRT | WO2017062672; SEQ ID NO: 601 | 4905 |
| CII2739 | FR | PRT | WO2017062672; SEQ ID NO: 189 | 4906 |
| CII2740 | FR | PRT | WO2017062672; SEQ ID NO: 798 | 4907 |
| CII2741 | FR | PRT | WO2017062672; SEQ ID NO: 801 | 4908 |
| CII2742 | FR | PRT | WO2017062672; SEQ ID NO: 600 | 4909 |
| CII2743 | FR | PRT | WO2017062672; SEQ ID NO: 805 | 4910 |
| CII2744 | FR | PRT | WO2017062672; SEQ ID NO: 803 | 4911 |
| CII2745 | FR | PRT | WO2017062672; SEQ ID NO: 113 | 4912 |
| CII2746 | FR | PRT | WO2017062672; SEQ ID NO: 590 | 4913 |
| CII2747 | FR | PRT | WO2017062672; SEQ ID NO: 591 | 4914 |
| CII2748 | FR | PRT | WO2017062672; SEQ ID NO: 791 | 4915 |
| CII2749 | FR | PRT | WO2017062672; SEQ ID NO: 720 | 4916 |
| CII2750 | FR | PRT | WO2017062672; SEQ ID NO: 786 | 4917 |
| CII2751 | FR | PRT | WO2017062672; SEQ ID NO: 161 | 4918 |
| CII2752 | FR | PRT | WO2017062672; SEQ ID NO: 163 | 4919 |
| CII2753 | FR | PRT | WO2017062672; SEQ ID NO: 788 | 4920 |
| CII2754 | FR | PRT | WO2017062672; SEQ ID NO: 158 | 4921 |
| CII2755 | FR | PRT | WO2017062672; SEQ ID NO: 171 | 4922 |
| CII2756 | FR | PRT | WO2017062672; SEQ ID NO: 167 | 4923 |
| CII2757 | FR | PRT | WO2017062672; SEQ ID NO: 598 | 4924 |
| CII2758 | FR | PRT | WO2017062672; SEQ ID NO: 169 | 4925 |
| CII2759 | FR | PRT | WO2017062672; SEQ ID NO: 597 | 4926 |
| CII2760 | FR | PRT | WO2017062672; SEQ ID NO: 792 | 4927 |
| CII2761 | FR | PRT | WO2017062672; SEQ ID NO: 790 | 4928 |
| CII2762 | FR | PRT | WO2017062672; SEQ ID NO: 787 | 4929 |
| CII2763 | FR | PRT | WO2017062672; SEQ ID NO: 173 | 4930 |
| CII2764 | FR | PRT | WO2017062672; SEQ ID NO: 806 | 4931 |
| CII2765 | FR | PRT | WO2017062672; SEQ ID NO: 187 | 4932 |
| CII2766 | FR | PRT | WO2017062672; SEQ ID NO: 104 | 4933 |
| CII2767 | FR | PRT | WO2017062672; SEQ ID NO: 119 | 4934 |
| CII2768 | FR | PRT | WO2017062672; SEQ ID NO: 108 | 4935 |
| CII2769 | FR | PRT | WO2017062672; SEQ ID NO: 106 | 4936 |
| CII2770 | FR | PRT | WO2017062672; SEQ ID NO: 777 | 4937 |
| CII2771 | FR | PRT | WO2017062672; SEQ ID NO: 807 | 4938 |
| CII2772 | FR | PRT | WO2017062672; SEQ ID NO: 216 | 4939 |
| CII2773 | FR | PRT | WO2017062672; SEQ ID NO: 218 | 4940 |
| CII2774 | FR | PRT | WO2017062672; SEQ ID NO: 215 | 4941 |
| CII2775 | FR | PRT | WO2017062672; SEQ ID NO: 209 | 4942 |
| CII2776 | FR | PRT | WO2017062672; SEQ ID NO: 214 | 4943 |
| CII2777 | FR | PRT | WO2017062672; SEQ ID NO: 210 | 4944 |
| CII2778 | FR | PRT | WO2017062672; SEQ ID NO: 211 | 4945 |
| CII2779 | FR | PRT | WO2017062672; SEQ ID NO: 217 | 4946 |
| CII2780 | FR | PRT | WO2017062672; SEQ ID NO: 213 | 4947 |
| CII2781 | FR | PRT | WO2017062672; SEQ ID NO: 212 | 4948 |
| CII2782 | FR | PRT | WO2017062672; SEQ ID NO: 721 | 4949 |
| CII2783 | FR | PRT | WO2017062672; SEQ ID NO: 122 | 4950 |
| CII2784 | FR | PRT | WO2017062672; SEQ ID NO: 186 | 4951 |
| CII2785 | FR | PRT | WO2017062672; SEQ ID NO: 174 | 4952 |
| CII2786 | FR | PRT | WO2017062672; SEQ ID NO: 808 | 4953 |
| CII2787 | FR | PRT | WO2017062672; SEQ ID NO: 797 | 4954 |
| CII2788 | FR | PRT | WO2017062672; SEQ ID NO: 794 | 4955 |
| CII2789 | FR | PRT | WO2017062672; SEQ ID NO: 795 | 4956 |
| CII2790 | FR | PRT | WO2017062672; SEQ ID NO: 184 | 4957 |
| CII2791 | FR | PRT | WO2017062672; SEQ ID NO: 796 | 4958 |
| CII2792 | FR | PRT | WO2017062672; SEQ ID NO: 181 | 4959 |
| CII2793 | FR | PRT | WO2017062672; SEQ ID NO: 180 | 4960 |
| CII2794 | FR | PRT | WO2017062672; SEQ ID NO: 183 | 4961 |
| CII2795 | FR | PRT | WO2017062672; SEQ ID NO: 176 | 4962 |
| CII2796 | FR | PRT | WO2017062672; SEQ ID NO: 793 | 4963 |
| CII2797 | FR | PRT | WO2017062672; SEQ ID NO: 177 | 4964 |
| CII2798 | FR | PRT | WO2017062672; SEQ ID NO: 179 | 4965 |
| CII2799 | FR | PRT | WO2017062672; SEQ ID NO: 178 | 4966 |
| CII2800 | FR | PRT | WO2017062672; SEQ ID NO: 175 | 4967 |
| CII2801 | FR | PRT | WO2017062672; SEQ ID NO: 185 | 4968 |
| CII2802 | FR | PRT | WO2017062672; SEQ ID NO: 128 | 4969 |
| CII2803 | FR | PRT | WO2017062672; SEQ ID NO: 124 | 4970 |
| CII2804 | FR | PRT | WO2017062672; SEQ ID NO: 125 | 4971 |
| CII2805 | FR | PRT | WO2017062672; SEQ ID NO: 120 | 4972 |
| CII2806 | FR | PRT | WO2017062672; SEQ ID NO: 778 | 4973 |
| CII2807 | FR | PRT | WO2017062672; SEQ ID NO: 129 | 4974 |
| CII2808 | FR | PRT | WO2017062672; SEQ ID NO: 593 | 4975 |
| CII2809 | FR | PRT | WO2017062672; SEQ ID NO: 121 | 4976 |
| CII2810 | FR | PRT | WO2017062672; SEQ ID NO: 123 | 4977 |
| CII2811 | FR | PRT | WO2017062672; SEQ ID NO: 126 | 4978 |
| CII2812 | FR | PRT | WO2017062672; SEQ ID NO: 779 | 4979 |
| CII2813 | FR | PRT | WO2017062672; SEQ ID NO: 127 | 4980 |
| CII2814 | FR | PRT | WO2017062672; SEQ ID NO: 780 | 4981 |
| CII2815 | FR | PRT | WO2017062672; SEQ ID NO: 162 | 4982 |
| CII2816 | FR | PRT | WO2017062672; SEQ ID NO: 771 | 4983 |
| CII2817 | FR | PRT | WO2017062672; SEQ ID NO: 789 | 4984 |
| CII2818 | Full antibody | PRT | US20190092843; SEQ ID NO: 1 | 4985 |
| CII2819 | Full antibody | PRT | WO2017075432; SEQ ID NO: 234 | 4986 |
| CII2820 | Full antibody | PRT | WO2017075432; SEQ ID NO: 233 | 4987 |
| CII2821 | Full antibody | PRT | WO2017075432; SEQ ID NO: 232 | 4988 |
| CII2822 | Full antibody | PRT | WO2017075432; SEQ ID NO: 228 | 4989 |
| CII2823 | Full antibody | PRT | WO2017075432; SEQ ID NO: 231 | 4990 |
| CII2824 | Full antibody | PRT | WO2017075432; SEQ ID NO: 230 | 4991 |
| CII2825 | Full antibody | PRT | WO2017075432; SEQ ID NO: 213 | 4992 |
| CII2826 | Full antibody | PRT | WO2017075432; SEQ ID NO: 216 | 4993 |
| CII2827 | Full antibody | PRT | WO2017075432; SEQ ID NO: 217 | 4994 |
| CII2828 | Full antibody | PRT | WO2017075432; SEQ ID NO: 219 | 4995 |
| CII2829 | Full antibody | PRT | WO2017075432; SEQ ID NO: 218 | 4996 |
| CII2830 | Full antibody | PRT | WO2017075432; SEQ ID NO: 215 | 4997 |
| CII2831 | Full antibody | PRT | WO2017075432; SEQ ID NO: 222 | 4998 |
| CII2832 | Full antibody | PRT | WO2017075432; SEQ ID NO: 237 | 4999 |
| CII2833 | Full antibody | PRT | WO2017075432; SEQ ID NO: 240 | 5000 |
| CII2834 | Full antibody | PRT | WO2017075432; SEQ ID NO: 239 | 5001 |
| CII2835 | Full antibody | PRT | WO2017075432; SEQ ID NO: 235 | 5002 |
| CII2836 | Full antibody | PRT | WO2017075432; SEQ ID NO: 238 | 5003 |
| CII2837 | Full antibody | PRT | WO2017075432; SEQ ID NO: 227 | 5004 |
| CII2838 | Full antibody | PRT | WO2017075432; SEQ ID NO: 226 | 5005 |
| CII2839 | Full antibody | PRT | WO2017075432; SEQ ID NO: 224 | 5006 |
| CII2840 | Full antibody | PRT | WO2017075432; SEQ ID NO: 225 | 5007 |
| CII2841 | Full antibody | PRT | WO2017075432; SEQ ID NO: 220 | 5008 |
| CII2842 | Full antibody | PRT | WO2017075432; SEQ ID NO: 223 | 5009 |
| CII2843 | Full antibody | PRT | WO2018107058; SEQ ID NO: 34 | 5010 |
| CII2844 | Full antibody | PRT | WO2017062672; SEQ ID NO: 870 | 5011 |
| CII2845 | Full antibody | PRT | WO2017062672; SEQ ID NO: 873 | 5012 |
| CII2846 | Full antibody | PRT | WO2017062672; SEQ ID NO: 874 | 5013 |
| CII2847 | Full antibody | PRT | WO2017062672; SEQ ID NO: 872 | 5014 |
| CII2848 | Full antibody | PRT | WO2017062672; SEQ ID NO: 875 | 5015 |
| CII2849 | Full antibody | PRT | WO2017062672; SEQ ID NO: 878 | 5016 |
| CII2850 | Full antibody | PRT | WO2017062672; SEQ ID NO: 882 | 5017 |
| CII2851 | Full antibody | PRT | WO2017062672; SEQ ID NO: 881 | 5018 |
| CII2852 | Full antibody | PRT | WO2017062672; SEQ ID NO: 880 | 5019 |
| CII2853 | Full antibody | PRT | WO2017062672; SEQ ID NO: 876 | 5020 |
| CII2854 | Full antibody | PRT | WO2017062672; SEQ ID NO: 879 | 5021 |
| CII2855 | Full antibody | PRT | WO2016201388; SEQ ID NO: 248 | 5022 |
| CII2856 | HC | DNA | US20170253653; SEQ ID NO: 30 | 5023 |
| CII2857 | HC | DNA | US20180051086; SEQ ID NO: 1 | 5024 |
| CII2858 | HC | PRT | US20130243775; SEQ ID NO: 5 | 5025 |
| CII2859 | HC | PRT | WO2019028283; SEQ ID NO: 166 | 5026 |
| CII2860 | HC | PRT | WO2019028283; SEQ ID NO: 23 | 5027 |
| CII2861 | HC | PRT | WO2019028283; SEQ ID NO: 22 | 5028 |
| CII2862 | HC | PRT | WO2019028283; SEQ ID NO: 26 | 5029 |
| CII2863 | HC | PRT | WO2019028283; SEQ ID NO: 25 | 5030 |
| CII2864 | HC | PRT | WO2019028283; SEQ ID NO: 167 | 5031 |
| CII2865 | HC | PRT | WO2019028283; SEQ ID NO: 24 | 5032 |
| CII2866 | HC | PRT | WO2019028283; SEQ ID NO: 4 | 5033 |
| CII2867 | HC | PRT | WO2019028283; SEQ ID NO: 179 | 5034 |
| CII2868 | HC | PRT | WO2019028283; SEQ ID NO: 200 | 5035 |
| CII2869 | HC | PRT | WO2019028283; SEQ ID NO: 182 | 5036 |
| CII2870 | HC | PRT | WO2019028283; SEQ ID NO: 203 | 5037 |
| CII2871 | HC | PRT | WO2019028283; SEQ ID NO: 176 | 5038 |
| CII2872 | HC | PRT | WO2019028283; SEQ ID NO: 197 | 5039 |
| CII2873 | HC | PRT | WO2019028283; SEQ ID NO: 181 | 5040 |
| CII2874 | HC | PRT | WO2019028283; SEQ ID NO: 202 | 5041 |
| CII2875 | HC | PRT | WO2019028283; SEQ ID NO: 184 | 5042 |
| CII2876 | HC | PRT | WO2019028283; SEQ ID NO: 205 | 5043 |
| CII2877 | HC | PRT | WO2019028283; SEQ ID NO: 178 | 5044 |
| CII2878 | HC | PRT | WO2019028283; SEQ ID NO: 199 | 5045 |
| CII2879 | HC | PRT | WO2019028283; SEQ ID NO: 180 | 5046 |
| CII2880 | HC | PRT | WO2019028283; SEQ ID NO: 201 | 5047 |
| CII2881 | HC | PRT | WO2019028283; SEQ ID NO: 183 | 5048 |
| CII2882 | HC | PRT | WO2019028283; SEQ ID NO: 204 | 5049 |
| CII2883 | HC | PRT | WO2019028283; SEQ ID NO: 177 | 5050 |
| CII2884 | HC | PRT | WO2019028283; SEQ ID NO: 198 | 5051 |
| CII2885 | HC | PRT | WO2019028283; SEQ ID NO: 2 | 5052 |
| CII2886 | HC | PRT | WO2019028283; SEQ ID NO: 3 | 5053 |
| CII2887 | HC | PRT | WO2019028283; SEQ ID NO: 158 | 5054 |
| CII2888 | HC | PRT | WO2019028283; SEQ ID NO: 9 | 5055 |
| CII2889 | HC | PRT | WO2019028283; SEQ ID NO: 21 | 5056 |
| CII2890 | HC | PRT | WO2019028283; SEQ ID NO: 20 | 5057 |
| CII2891 | HC | PRT | WO2019028283; SEQ ID NO: 160 | 5058 |
| CII2892 | HC | PRT | WO2019028283; SEQ ID NO: 5 | 5059 |
| CII2893 | HC | PRT | WO2019028283; SEQ ID NO: 159 | 5060 |
| CII2894 | HC | PRT | WO2019028283; SEQ ID NO: 161 | 5061 |
| CII2895 | HC | PRT | WO2019028283; SEQ ID NO: 13 | 5062 |
| CII2896 | HC | PRT | WO2019028283; SEQ ID NO: 7 | 5063 |
| CII2897 | HC | PRT | WO2019028283; SEQ ID NO: 10 | 5064 |
| CII2898 | HC | PRT | WO2019028283; SEQ ID NO: 14 | 5065 |
| CII2899 | HC | PRT | WO2019028283; SEQ ID NO: 8 | 5066 |
| CII2900 | HC | PRT | WO2019028283; SEQ ID NO: 19 | 5067 |
| CII2901 | HC | PRT | WO2019028283; SEQ ID NO: 18 | 5068 |
| CII2902 | HC | PRT | WO2019028283; SEQ ID NO: 12 | 5069 |
| CII2903 | HC | PRT | WO2019028283; SEQ ID NO: 17 | 5070 |
| CII2904 | HC | PRT | WO2019028283; SEQ ID NO: 16 | 5071 |
| CII2905 | HC | PRT | WO2019028283; SEQ ID NO: 6 | 5072 |
| CII2906 | HC | PRT | WO2019028283; SEQ ID NO: 15 | 5073 |
| CII2907 | HC | PRT | WO2019028283; SEQ ID NO: 11 | 5074 |
| CII2908 | HC | PRT | WO2019079549; SEQ ID NO: 1 | 5075 |
| CII2909 | HC | PRT | US20180333503; SEQ ID NO: 13 | 5076 |
| CII2910 | HC | PRT | US20180333503; SEQ ID NO: 3 | 5077 |
| CII2911 | HC | PRT | US20190117769; SEQ ID NO: 14 | 5078 |
| CII2912 | HC | PRT | US20190117769; SEQ ID NO: 12 | 5079 |
| CII2913 | HC | PRT | US20190117769; SEQ ID NO: 16 | 5080 |
| CII2914 | HC | PRT | US20180333503; SEQ ID NO: 28 | 5081 |
| CII2915 | HC | PRT | US20170253653; SEQ ID NO: 26 | 5082 |
| CII2916 | HC | PRT | US20170253653; SEQ ID NO: 34 | 5083 |
| CII2917 | HC | PRT | US20170253653; SEQ ID NO: 24 | 5084 |
| CII2918 | HC | PRT | US20170253653; SEQ ID NO: 32 | 5085 |
| CII2919 | HC | PRT | WO2019079549; SEQ ID NO: 3 | 5086 |
| CII2920 | HC | PRT | US20190016807; SEQ ID NO: 23 | 5087 |
| CII2921 | HC | PRT | US20199016807; SEQ ID NO: 24 | 5088 |
| CII2922 | HC | PRT | US20190016807; SEQ ID NO: 22 | 5089 |
| CII2923 | HC | PRT | WO2018213316; SEQ ID NO: 130 | 5090 |
| CII2924 | HC | PRT | WO2018140121: WO2018147927; SEQ | 5091 |
| | | | ID NO: 36 | |
| CII2925 | HC | PRT | WO2016023019; SEQ ID NO: 397 | 5092 |
| CII2926 | HC | PRT | WO2019028292; SEQ ID NO: 20 | 5093 |
| CII2927 | HC | PRT | WO2019028292; SEQ ID NO: 17 | 5094 |
| CII2928 | HC | PRT | WO2019028292; SEQ ID NO: 10 | 5095 |
| CII2929 | HC | PRT | WO2019028292; SEQ ID NO: 19 | 5096 |
| CII2930 | HC | PRT | WO2019028292; SEQ ID NO: 18 | 5097 |
| CII2931 | HC | PRT | WO2019028292; SEQ ID NO: 11 | 5098 |
| CII2932 | HC | PRT | WO2019028292; SEQ ID NO: 198 | 5099 |
| CII2933 | HC | PRT | WO2019028292; SEQ ID NO: 199 | 5100 |
| CII2934 | HC | PRT | WO2019028292; SEQ ID NO: 200 | 5101 |
| CII2935 | HC | PRT | WO2019028292; SEQ ID NO: 201 | 5102 |
| CII2936 | HC | PRT | WO2019028292; SEQ ID NO: 9 | 5103 |
| CII2937 | HC | PRT | WO2019028292; SEQ ID NO: 202 | 5104 |
| CII2938 | HC | PRT | WO2019028292; SEQ ID NO: 203 | 5105 |
| CII2939 | HC | PRT | WO2019028292; SEQ ID NO: 204 | 5106 |
| CII2940 | HC | PRT | WO2019028292; SEQ ID NO: 205 | 5107 |
| CII2941 | HC | PRT | WO2019028292; SEQ ID NO: 212 | 5108 |
| CII2942 | HC | PRT | WO2019028292; SEQ ID NO: 213 | 5109 |
| CII2943 | HC | PRT | WO2019028292; SEQ ID NO: 208 | 5110 |
| CII2944 | HC | PRT | WO2019028292; SEQ ID NO: 209 | 5111 |
| CII2945 | HC | PRT | WO2019028292; SEQ ID NO: 210 | 5112 |
| CII2946 | HC | PRT | WO2019028292; SEQ ID NO: 211 | 5113 |
| CII2947 | HC | PRT | WO2019028292; SEQ ID NO: 206 | 5114 |
| CII2948 | HC | PRT | WO2019028292; SEQ ID NO: 207 | 5115 |
| CII2949 | HC | PRT | WO2019028292; SEQ ID NO: 14 | 5116 |
| CII2950 | HC | PRT | WO2019028292; SEQ ID NO: 15 | 5117 |
| CII2951 | HC | PRT | WO2019028292; SEQ ID NO: 16 | 5118 |
| CII2952 | HC | PRT | WO2019028292; SEQ ID NO: 12 | 5119 |
| CII2953 | HC | PRT | WO2019028292; SEQ ID NO: 13 | 5120 |
| CII2954 | HC | PRT | US20190137523; SEQ ID NO: 5 | 5121 |
| CII2955 | HC | PRT | US20190137523; SEQ ID NO: 3 | 5122 |
| CII2956 | HC | PRT | US20180221480; SEQ ID NO: 1 | 5123 |
| CII2957 | HC | PRT | WO2018140510; SEQ ID NO: 6 | 5124 |
| CII2958 | LC | DNA | US20170253653; SEQ ID NO: 31 | 5125 |
| CII2959 | LC | DNA | US20180051086; SEQ ID NO: 3 | 5126 |
| CII2960 | LC | PRT | WO2019028283; SEQ ID NO: 185 | 5127 |
| CII2961 | LC | PRT | WO2019028283; SEQ ID NO: 33 | 5128 |
| CII2962 | LC | PRT | WO2019028283; SEQ ID NO: 32 | 5129 |
| CII2963 | LC | PRT | WO2019028283; SEQ ID NO: 31 | 5130 |
| CII2964 | LC | PRT | WO2019028283; SEQ ID NO: 29 | 5131 |
| CII2965 | LC | PRT | WO2019028283; SEQ ID NO: 30 | 5132 |
| CII2966 | LC | PRT | WO2019028283; SEQ ID NO: 163 | 5133 |
| CII2967 | LC | PRT | WO2019028283; SEQ ID NO: 27 | 5134 |
| CII2968 | LC | PRT | WO2019028283; SEQ ID NO: 28 | 5135 |
| CII2969 | LC | PRT | WO2019028283; SEQ ID NO: 162 | 5136 |
| CII2970 | LC | PRT | WO2019028283; SEQ ID NO: 168 | 5137 |
| CII2971 | LC | PRT | WO2019028283; SEQ ID NO: 169 | 5138 |
| CII2972 | LC | PRT | WO2019079549; SEQ ID NO: 2 | 5139 |
| CII2973 | LC | PRT | US20180333503; SEQ ID NO: 15 | 5140 |
| CII2974 | LC | PRT | US20190117769; SEQ ID NO: 11 | 5141 |
| CII2975 | LC | PRT | US20190117769; SEQ ID NO: 13 | 5142 |
| CII2976 | LC | PRT | US20190117769; SEQ ID NO: 15 | 5143 |
| CII2977 | LC | PRT | US20180333503; SEQ ID NO: 29 | 5144 |
| CII2978 | LC | PRT | US20190016807; SEQ ID NO: 34 | 5145 |
| CII2979 | LC | PRT | US20190016807; SEQ ID NO: 30 | 5146 |
| CII2980 | LC | PRT | US20190016807; SEQ ID NO: 35 | 5147 |
| CII2981 | LC | PRT | US20190016807; SEQ ID NO: 36 | 5148 |
| CII2982 | LC | PRT | US20190016807; SEQ ID NO: 37 | 5149 |
| CII2983 | LC | PRT | US20190016807; SEQ ID NO: 40 | 5150 |
| CII2984 | LC | PRT | US29190016807; SEQ ID NO: 32 | 5151 |
| CII2985 | LC | PRT | US20190016807; SEQ ID NO: 38 | 5152 |
| CII2986 | LC | PRT | US20190016807; SEQ ID NO: 31 | 5153 |
| CII2987 | LC | PRT | US20190016807; SEQ ID NO: 21 | 5154 |
| CII2988 | LC | PRT | US20190016807; SEQ ID NO: 39 | 5155 |
| CII2989 | LC | PRT | US20190016807; SEQ ID NO: 27 | 5156 |
| CII2990 | LC | PRT | US20190016807; SEQ ID NO: 26 | 5157 |
| CII2991 | LC | PRT | US20190016807; SEQ ID NO: 28 | 5158 |
| CII2992 | LC | PRT | US20190016807; SEQ ID NO: 33 | 5159 |
| CII2993 | LC | PRT | US20190016807; SEQ ID NO: 25 | 5160 |
| CII2994 | LC | PRT | US20190016807; SEQ ID NO: 29 | 5161 |
| CII2995 | LC | PRT | US20170253653; SEQ ID NO: 27 | 5162 |
| CII2996 | LC | PRT | US20170253653; SEQ ID NO: 35 | 5163 |
| CII2997 | LC | PRT | US20170253653; SEQ ID NO: 25 | 5164 |
| CII2998 | LC | PRT | US20170253653; SEQ ID NO: 33 | 5165 |
| CII2999 | LC | PRT | WO2019079549; SEQ ID NO: 4 | 5166 |
| CII3000 | LC | PRT | WO2018140121; WO2018147928; SEQ | 5167 |
| | | | ID NO: 37 | |
| CII3001 | LC | PRT | WO2019028292; SEQ ID NO: 214 | 5168 |
| CII3002 | LC | PRT | WO2019028292; SEQ ID NO: 215 | 5169 |
| CII3003 | LC | PRT | WO2019028292; SEQ ID NO: 26 | 5170 |
| CII3004 | LC | PRT | WO2019028292; SEQ ID NO: 25 | 5171 |
| CII3005 | LC | PRT | WO2019028292; SEQ ID NO: 23 | 5172 |
| CII3006 | LC | PRT | WO2019028292; SEQ ID NO: 24 | 5173 |
| CII3007 | LC | PRT | WO2019028292; SEQ ID NO: 216 | 5174 |
| CII3008 | LC | PRT | WO2019028292; SEQ ID NO: 217 | 5175 |
| CII3009 | LC | PRT | WO2019028292; SEQ ID NO: 218 | 5176 |
| CII3010 | LC | PRT | WO2019028292; SEQ ID NO: 21 | 5177 |
| CII3011 | LC | PRT | WO2019028292; SEQ ID NO: 22 | 5178 |
| CII3012 | LC | PRT | US20190137523; SEQ ID NO: 2 | 5179 |
| CII3013 | LC | PRT | US20190137523; SEQ ID NO: 4 | 5180 |
| CII3014 | LC | PRT | US20180221480; SEQ ID NO: 8 | 5181 |
| CII3015 | LC | PRT | US20180221480; SEQ ID NO: 9 | 5182 |
| CII3016 | LC | PRT | US20180221480; SEQ ID NO: 11 | 5183 |
| CII3017 | LC | PRT | US20180221480; SEQ ID NO: 10 | 5184 |
| CII3018 | LC | PRT | US20180221480; SEQ ID NO: 6 | 5185 |
| CII3019 | LC | PRT | WO2018140510; SEQ ID NO: 1 | 5186 |
| CII3020 | VH | DNA | WO2018140121; WO2018147925; SEQ | 5187 |
| | | | ID NO: 34 | |
| CII3021 | VH | DNA | US20140212413; SEQ ID NO: 4 | 5188 |
| CII3022 | VH | DNA | WO2010129469; SEQ ID NO: 37 | 5189 |
| CII3023 | VH | PRT | US20190092843; SEQ ID NO: 2 | 5190 |
| CII3024 | VH | PRT | WO2014028776; SEQ ID NO: 62 | 5191 |
| CII3025 | VH | PRT | US20180134806; SEQ ID NO: 73 | 5192 |
| CII3026 | VH | PRT | US20180134806; SEQ ID NO: 76 | 5193 |
| CII3027 | VH | PRT | US20180134806; SEQ ID NO: 77 | 5194 |
| CII3028 | VH | PRT | US20180134806; SEQ ID NO: 74 | 5195 |
| CII3029 | VH | PRT | US20180134806; SEQ ID NO: 4 | 5196 |
| CII3030 | VH | PRT | US20130243775; SEQ ID NO: 3 | 5197 |
| CII3031 | VH | PRT | WO2019020606; SEQ ID NO: 14 | 5198 |
| CII3032 | VH | PRT | WO2019020606; SEQ ID NO: 16 | 5199 |
| CII3033 | VH | PRT | WO2019020606; SEQ ID NO: 12 | 5200 |
| CII3034 | VH | PRT | WO2019020606; SEQ ID NO: 19 | 5201 |
| CII3035 | VH | PRT | WO2019020606; SEQ ID NO: 11 | 5202 |
| CII3036 | VH | PRT | WO2019020606; SEQ ID NO: 18 | 5203 |
| CII3037 | VH | PRT | WO2019020606; SEQ ID NO: 17 | 5204 |
| CII3038 | VH | PRT | WO2019020606; SEQ ID NO: 15 | 5205 |
| CII3039 | VH | PRT | WO2019020606; SEQ ID NO: 13 | 5206 |
| CII3040 | VH | PRT | WO2019020606; SEQ ID NO: 10 | 5207 |
| CII3041 | VH | PRT | WO2019020606; SEQ ID NO: 9 | 5208 |
| CII3042 | VH | PRT | WO2019020606; SEQ ID NO: 5 | 5209 |
| CII3043 | VH | PRT | WO2019020606; SEQ ID NO: 7 | 5210 |
| CII3044 | VH | PRT | WO2019020606; SEQ ID NO: 8 | 5211 |
| CII3045 | VH | PRT | WO2019020606; SEQ ID NO: 4 | 5212 |
| CII3046 | VH | PRT | WO2019020606; SEQ ID NO: 3 | 5213 |
| CII3047 | VH | PRT | WO2019020606; SEQ ID NO: 6 | 5214 |
| CII3048 | VH | PRT | WO2016201388; SEQ ID NO: 245 | 5215 |
| CII3049 | VH | PRT | US20190085076; SEQ ID NO: 372 | 5216 |
| CII3050 | VH | PRT | W02016201389; SEQ ID NO: 372 | 5217 |
| CII3051 | VH | PRT | WO2016201388; SEQ ID NO: 161 | 5218 |
| CII3052 | VH | PRT | US20190085076; SEQ ID NO: 370 | 5219 |
| CII3053 | VH | PRT | W02016201389; SEQ ID NO: 370 | 5220 |
| CII3054 | VH | PRT | US20190085076; SEQ ID NO: 417 | 5221 |
| CII3055 | VH | PRT | W02016201389; SEQ ID NO: 417 | 5222 |
| CII3056 | VH | PRT | US20190085076; SEQ ID NO: 419 | 5223 |
| CII3057 | VH | PRT | W02016201389; SEQ ID NO: 419 | 5224 |
| CII3058 | VH | PRT | US20190085076; SEQ ID NO: 431 | 5225 |
| CII3059 | VH | PRT | W02016201389; SEQ ID NO: 431 | 5226 |
| CII3060 | VH | PRT | US20190085076; SEQ ID NO: 363 | 5227 |
| CII3061 | VH | PRT | W02016201389; SEQ ID NO: 363 | 5228 |
| CII3062 | VH | PRT | US20190085076; SEQ ID NO: 380 | 5229 |
| CII3063 | VH | PRT | W02016201389; SEQ ID NO: 380 | 5230 |
| CII3064 | VH | PRT | US20190085076; SEQ ID NO: 430 | 5231 |
| CII3065 | VH | PRT | W02016201389; SEQ ID NO: 430 | 5232 |
| CII3066 | VH | PRT | US20190085076; SEQ ID NO: 432 | 5233 |
| CII3067 | VH | PRT | W02016201389; SEQ ID NO: 432 | 5234 |
| CII3068 | VH | PRT | US20190085076; SEQ ID NO: 367 | 5235 |
| CII3069 | VH | PRT | W02016201389; SEQ ID NO: 367 | 5236 |
| CII3070 | VH | PRT | US20190085076; SEQ ID NO: 418 | 5237 |
| CII3071 | VH | PRT | W02016201389; SEQ ID NO: 418 | 5238 |
| CII3072 | VH | PRT | US20190085076; SEQ ID NO: 364 | 5239 |
| CII3073 | VH | PRT | W02016201389; SEQ ID NO: 364 | 5240 |
| CII3074 | VH | PRT | US20190085076; SEQ ID NO: 366 | 5241 |
| CII3075 | VH | PRT | W02016201389; SEQ ID NO: 366 | 5242 |
| CII3076 | VH | PRT | US20190085076; SEQ ID NO: 403 | 5243 |
| CII3077 | VH | PRT | W02016201389; SEQ ID NO: 403 | 5244 |
| CII3078 | VH | PRT | US20190085076; SEQ ID NO: 420 | 5245 |
| CII3079 | VH | PRT | W02016201389; SEQ ID NO: 420 | 5246 |
| CII3080 | VH | PRT | WO2016201388; SEQ ID NO: 63 | 5247 |
| CII3081 | VH | PRT | WO2016201388; SEQ ID NO: 57 | 5248 |
| CII3082 | VH | PRT | WO2016201388; SEQ ID NO: 52 | 5249 |
| CII3083 | VH | PRT | WO2016201388; SEQ ID NO: 206 | 5250 |
| CII3084 | VH | PRT | WO2016201388; SEQ ID NO: 173 | 5251 |
| CII3085 | VH | PRT | WO2016201388; SEQ ID NO: 154 | 5252 |
| CII3086 | VH | PRT | WO2016201388; SEQ ID NO: 244 | 5253 |
| CII3087 | VH | PRT | WO2019028283; SEQ ID NO: 103 | 5254 |
| CII3088 | VH | PRT | WO2016201388; SEQ ID NO: 246 | 5255 |
| CII3089 | VH | PRT | WO2016201388; SEQ ID NO: 66 | 5256 |
| CII3090 | VH | PRT | US20190085076; SEQ ID NO: 429 | 5257 |
| CII3091 | VH | PRT | W02016201389; SEQ ID NO: 429 | 5258 |
| CII3092 | VH | PRT | WO2016201388; SEQ ID NO: 158 | 5259 |
| CII3093 | VH | PRT | WO2016201388; SEQ ID NO: 181 | 5260 |
| CII3094 | VH | PRT | WO2016201388; SEQ ID NO: 212 | 5261 |
| CII3095 | VH | PRT | WO2016201388; SEQ ID NO: 176 | 5262 |
| CII3096 | VH | PRT | WO2016201388; SEQ ID NO: 160 | 5263 |
| CII3097 | VH | PRT | US20190085076; SEQ ID NO: 406 | 5264 |
| CII3098 | VH | PRT | W02016201389; SEQ ID NO: 406 | 5265 |
| CII3099 | VH | PRT | US20190085076; SEQ ID NO: 393 | 5266 |
| CII3100 | VH | PRT | W02016201389; SEQ ID NO: 393 | 5267 |
| CII3101 | VH | PRT | US20190085076; SEQ ID NO: 405 | 5268 |
| CII3102 | VH | PRT | W02016201389; SEQ ID NO: 405 | 5269 |
| CII3103 | VH | PRT | US20190085076; SEQ ID NO: 397 | 5270 |
| CII3104 | VH | PRT | W02016201389; SEQ ID NO: 397 | 5271 |
| CII3105 | VH | PRT | US20190085076; SEQ ID NO: 413 | 5272 |
| CII3106 | VH | PRT | W02016201389; SEQ ID NO: 413 | 5273 |
| CII3107 | VH | PRT | US20190085076; SEQ ID NO: 428 | 5274 |
| CII3108 | VH | PRT | W02016201389; SEQ ID NO: 428 | 5275 |
| CII3109 | VH | PRT | US20190085076; SEQ ID NO: 384 | 5276 |
| CII3110 | VH | PRT | W02016201389; SEQ ID NO: 384 | 5277 |
| CII3111 | VH | PRT | US20190085076; SEQ ID NO: 390 | 5278 |
| CII3112 | VH | PRT | W02016201389; SEQ ID NO: 399 | 5279 |
| CII3113 | VH | PRT | US20190085076; SEQ ID NO: 389 | 5280 |
| CII3114 | VH | PRT | W02016201389; SEQ ID NO: 389 | 5281 |
| CII3115 | VH | PRT | US20190085076; SEQ ID NO: 373 | 5282 |
| CII3116 | VH | PRT | W02016201389; SEQ ID NO: 373 | 5283 |
| CII3117 | VH | PRT | US20190085076; SEQ ID NO: 391 | 5284 |
| CII3118 | VH | PRT | W02016201389; SEQ ID NO: 391 | 5285 |
| CII3119 | VH | PRT | US20190085076; SEQ ID NO: 427 | 5286 |
| CII3120 | VH | PRT | W02016201389; SEQ ID NO: 427 | 5287 |
| CII3121 | VH | PRT | US20190085076; SEQ ID NO: 386 | 5288 |
| CII3122 | VH | PRT | W02016201389; SEQ ID NO: 386 | 5289 |
| CII3123 | VH | PRT | US20190085076; SEQ ID NO: 376 | 5290 |
| CII3124 | VH | PRT | W02016201389; SEQ ID NO: 376 | 5291 |
| CII3125 | VH | PRT | WO2016201388; SEQ ID NO: 155 | 5292 |
| CII3126 | VH | PRT | WO2016201388; SEQ ID NO: 178 | 5293 |
| CII3127 | VH | PRT | WO2016201388; SEQ ID NO: 207 | 5294 |
| CII3128 | VH | PRT | WO2016201388; SEQ ID NO: 209 | 5295 |
| CII3129 | VH | PRT | WO2016201388; SEQ ID NO: 171 | 5296 |
| CII3130 | VH | PRT | WO2016201388; SEQ ID NO: 165 | 5297 |
| CII3131 | VH | PRT | WO2016201388; SEQ ID NO: 64 | 5298 |
| CII3132 | VH | PRT | WO2016201388; SEQ ID NO: 58 | 5299 |
| CII3133 | VH | PRT | WO2016201388; SEQ ID NO: 53 | 5300 |
| CII3134 | VH | PRT | WO2016201388; SEQ ID NO: 205 | 5301 |
| CII3135 | VH | PRT | WO2016201388; SEQ ID NO: 170 | 5302 |
| CII3136 | VH | PRT | WO2016201388; SEQ ID NO: 167 | 5303 |
| CII3137 | VH | PRT | US20190085076; SEQ ID NO: 385 | 5304 |
| CII3138 | VH | PRT | W02016201389; SEQ ID NO: 385 | 5305 |
| CII3139 | VH | PRT | US20190085076; SEQ ID NO: 392 | 5306 |
| CII3140 | VH | PRT | W02016201389; SEQ ID NO: 392 | 5307 |
| CII3141 | VH | PRT | US20190085076; SEQ ID NO: 424 | 5308 |
| CII3142 | VH | PRT | W02016201389; SEQ ID NO: 424 | 5309 |
| CII3143 | VH | PRT | US20190085076; SEQ ID NO: 400 | 5310 |
| CII3144 | VH | PRT | W02016201389; SEQ ID NO: 400 | 5311 |
| CII3145 | VH | PRT | US29190085076; SEQ ID NO: 379 | 5312 |
| CII3146 | VH | PRT | W02016201389; SEQ ID NO: 379 | 5313 |
| CII3147 | VH | PRT | US20190085076; SEQ ID NO: 398 | 5314 |
| CII3148 | VH | PRT | W02016201389; SEQ ID NO: 398 | 5315 |
| CII3149 | VH | PRT | US20190085076; SEQ ID NO: 394 | 5316 |
| CII3150 | VH | PRT | W02016201389; SEQ ID NO: 394 | 5317 |
| CII3151 | VH | PRT | US20190085076; SEQ ID NO: 395 | 5318 |
| CII3152 | VH | PRT | W02016201389; SEQ ID NO: 395 | 5319 |
| CII3153 | VH | PRT | US20199085076; SEQ ID NO: 426 | 5320 |
| CII3154 | VH | PRT | W02016201389; SEQ ID NO: 426 | 5321 |
| CII3155 | VH | PRT | US20190085076; SEQ ID NO: 396 | 5322 |
| CII3156 | VH | PRT | W02016201389; SEQ ID NO: 396 | 5323 |
| CII3157 | VH | PRT | US20190085076; SEQ ID NO: 399 | 5324 |
| CII3158 | VH | PRT | W02016201389; SEQ ID NO: 399 | 5325 |
| CII3159 | VH | PRT | US20199085076; SEQ ID NO: 371 | 5326 |
| CII3160 | VH | PRT | W02016201389; SEQ ID NO: 371 | 5327 |
| CII3161 | VH | PRT | US20190085076; SEQ ID NO: 410 | 5328 |
| CII3162 | VH | PRT | W02016201389; SEQ ID NO: 410 | 5329 |
| CII3163 | VH | PRT | US20190085076; SEQ ID NO: 411 | 5330 |
| CII3164 | VH | PRT | W02016201389; SEQ ID NO: 411 | 5331 |
| CII3165 | VH | PRT | US20190085076; SEQ ID NO: 404 | 5332 |
| CII3166 | VH | PRT | W02016201389; SEQ ID NO: 404 | 5333 |
| CII3167 | VH | PRT | US29190085076; SEQ ID NO: 425 | 5334 |
| CII3168 | VH | PRT | W02016201389; SEQ ID NO: 425 | 5335 |
| CII3169 | VH | PRT | WO2016201388; SEQ ID NO: 183 | 5336 |
| CII3170 | VH | PRT | WO2016291388; SEQ ID NO: 182 | 5337 |
| CII3171 | VH | PRT | US20190085076; SEQ ID NO: 381 | 5338 |
| CII3172 | VH | PRT | W02016201389; SEQ ID NO: 381 | 5339 |
| CII3173 | VH | PRT | US20190085076; SEQ ID NO: 382 | 5340 |
| CII3174 | VH | PRT | W02016201389; SEQ ID NO: 382 | 5341 |
| CII3175 | VH | PRT | WO2016201388; SEQ ID NO: 166 | 5342 |
| CII3176 | VH | PRT | WO2016201388; SEQ ID NO: 157 | 5343 |
| CII3177 | VH | PRT | WO2016201388; SEQ ID NO: 180 | 5344 |
| CII3178 | VH | PRT | WO2016201388; SEQ ID NO: 211 | 5345 |
| CII3179 | VH | PRT | WO2016201388; SEQ ID NO: 175 | 5346 |
| CII3180 | VH | PRT | US20190085076; SEQ ID NO: 412 | 5347 |
| CII3181 | VH | PRT | W02016201389; SEQ ID NO: 412 | 5348 |
| CII3182 | VH | PRT | US20190085076; SEQ ID NO: 377 | 5349 |
| CII3183 | VH | PRT | W02016201389; SEQ ID NO: 377 | 5350 |
| CII3184 | VH | PRT | US20190085076; SEQ ID NO: 388 | 5351 |
| CII3185 | VH | PRT | W02016201389; SEQ ID NO: 388 | 5352 |
| CII3186 | VH | PRT | WO2016201388; SEQ ID NO: 159 | 5353 |
| CII3187 | VH | PRT | WO2016201388; SEQ ID NO: 213 | 5354 |
| CII3188 | VH | PRT | WO2016201388; SEQ ID NO: 177 | 5355 |
| CII3189 | VH | PRT | WO2016201388; SEQ ID NO: 203 | 5356 |
| CII3190 | VH | PRT | WO2016201388; SEQ ID NO: 168 | 5357 |
| CII3191 | VH | PRT | WO2016201388; SEQ ID NO: 162 | 5358 |
| CII3192 | VH | PRT | US20190085076; SEQ ID NO: 402 | 5359 |
| CII3193 | VH | PRT | W02016201389; SEQ ID NO: 402 | 5360 |
| CII3194 | VH | PRT | US20190085076; SEQ ID NO: 415 | 5361 |
| CII3195 | VH | PRT | W02016201389; SEQ ID NO: 415 | 5362 |
| CII3196 | VH | PRT | US20190085076; SEQ ID NO: 375 | 5363 |
| CII3197 | VH | PRT | W02016201389; SEQ ID NO: 375 | 5364 |
| CII3198 | VH | PRT | US20190085076; SEQ ID NO: 416 | 5365 |
| CII3199 | VH | PRT | W02016201389; SEQ ID NO: 416 | 5366 |
| CII3200 | VH | PRT | US20190085076; SEQ ID NO: 414 | 5367 |
| CII3201 | VH | PRT | W02016201389; SEQ ID NO: 414 | 5368 |
| CII3202 | VH | PRT | WO2016201388; SEQ ID NO: 156 | 5369 |
| CII3203 | VH | PRT | WO2016201388; SEQ ID NO: 179 | 5370 |
| CII3204 | VH | PRT | WO2016201388; SEQ ID NO: 208 | 5371 |
| CII3205 | VH | PRT | WO2016201388; SEQ ID NO: 174 | 5372 |
| CII3206 | VH | PRT | WO2019028283; SEQ ID NO: 73 | 5373 |
| CII3207 | VH | PRT | WO2019028283; SEQ ID NO: 54 | 5374 |
| CII3208 | VH | PRT | WO2019028283; SEQ ID NO: 64 | 5375 |
| CII3209 | VH | PRT | WO2019028283; SEQ ID NO: 63 | 5376 |
| CII3210 | VH | PRT | WO2019028283; SEQ ID NO: 52 | 5377 |
| CII3211 | VH | PRT | WO2019028283; SEQ ID NO: 53 | 5378 |
| CII3212 | VH | PRT | WO2019028283; SEQ ID NO: 62 | 5379 |
| CII3213 | VH | PRT | WO2019028283; SEQ ID NO: 61 | 5380 |
| CII3214 | VH | PRT | WO2019028283; SEQ ID NO: 74 | 5381 |
| CII3215 | VH | PRT | WO2019028283; SEQ ID NO: 76 | 5382 |
| CII3216 | VH | PRT | WO2019028283; SEQ ID NO: 66 | 5383 |
| CII3217 | VH | PRT | WO2019028283; SEQ ID NO: 65 | 5384 |
| CII3218 | VH | PRT | WO2019028283; SEQ ID NO: 58 | 5385 |
| CII3219 | VH | PRT | WO2019028283; SEQ ID NO: 69 | 5386 |
| CII3220 | VH | PRT | WO2019028283; SEQ ID NO: 59 | 5387 |
| CII3221 | VH | PRT | WO2019028283; SEQ ID NO: 68 | 5388 |
| CII3222 | VH | PRT | WO2019028283; SEQ ID NO: 67 | 5389 |
| CII3223 | VH | PRT | WO2019028283; SEQ ID NO: 72 | 5390 |
| CII3224 | VH | PRT | WO2019028283; SEQ ID NO: 69 | 5391 |
| CII3225 | VH | PRT | WO2019028283; SEQ ID NO: 71 | 5392 |
| CII3226 | VH | PRT | WO2019028283; SEQ ID NO: 70 | 5393 |
| CII3227 | VH | PRT | WO2019028283; SEQ ID NO: 173 | 5394 |
| CII3228 | VH | PRT | WO2019028283; SEQ ID NO: 174 | 5395 |
| CII3229 | VH | PRT | WO2019028283; SEQ ID NO: 55 | 5396 |
| CII3230 | VH | PRT | WO2019028283; SEQ ID NO: 56 | 5397 |
| CII3231 | VH | PRT | WO2019028283; SEQ ID NO: 57 | 5398 |
| CII3232 | VH | PRT | WO2019028283; SEQ ID NO: 34 | 5399 |
| CII3233 | VH | PRT | WO2019028283; SEQ ID NO: 35 | 5400 |
| CII3234 | VH | PRT | WO2016201388; SEQ ID NO: 164 | 5401 |
| CII3235 | VH | PRT | WO2019028283; SEQ ID NO: 44 | 5402 |
| CII3236 | VH | PRT | WO2016201388; SEQ ID NO: 61 | 5403 |
| CII3237 | VH | PRT | WO2019028283; SEQ ID NO: 45 | 5404 |
| CII3238 | VH | PRT | WO2019028283; SEQ ID NO: 46 | 5405 |
| CII3239 | VH | PRT | WO2019028283; SEQ ID NO: 47 | 5406 |
| CII3240 | VH | PRT | WO2019028283; SEQ ID NO: 48 | 5407 |
| CII3241 | VH | PRT | WO2019028283; SEQ ID NO: 42 | 5408 |
| CII3242 | VH | PRT | WO2016201388; SEQ ID NO: 55 | 5409 |
| CII3243 | VH | PRT | WO2019028283; SEQ ID NO: 170 | 5410 |
| CII3244 | VH | PRT | WO2019028283; SEQ ID NO: 75 | 5411 |
| CII3245 | VH | PRT | WO2019028283; SEQ ID NO: 171 | 5412 |
| CII3246 | VH | PRT | WO2019028283; SEQ ID NO: 175 | 5413 |
| CII3247 | VH | PRT | WO2019028283; SEQ ID NO: 172 | 5414 |
| CII3248 | VH | PRT | WO2019028283; SEQ ID NO: 41 | 5415 |
| CII3249 | VH | PRT | WO2019028283; SEQ ID NO: 36 | 5416 |
| CII3250 | VH | PRT | WO2019028283; SEQ ID NO: 37 | 5417 |
| CII3251 | VH | PRT | WO2019028283; SEQ ID NO: 50 | 5418 |
| CII3252 | VH | PRT | WO2019028283; SEQ ID NO: 38 | 5419 |
| CII3253 | VH | PRT | WO2019028283; SEQ ID NO: 39 | 5420 |
| CII3254 | VH | PRT | WO2019028283; SEQ ID NO: 40 | 5421 |
| CII3255 | VH | PRT | US20190085076; SEQ ID NO: 423 | 5422 |
| CII3256 | VH | PRT | W02016201389; SEQ ID NO: 423 | 5423 |
| CII3257 | VH | PRT | US20190085076; SEQ ID NO: 401 | 5424 |
| CII3258 | VH | PRT | W02016201389; SEQ ID NO: 401 | 5425 |
| CII3259 | VH | PRT | US20190085076; SEQ ID NO: 369 | 5426 |
| CII3260 | VH | PRT | W02016201389; SEQ ID NO: 369 | 5427 |
| CII3261 | VH | PRT | US20190085076; SEQ ID NO: 422 | 5428 |
| CII3262 | VH | PRT | W02016201389; SEQ ID NO: 422 | 5429 |
| CII3263 | VH | PRT | US20190085076; SEQ ID NO: 368 | 5430 |
| CII3264 | VH | PRT | W02016201389; SEQ ID NO: 368 | 5431 |
| CII3265 | VH | PRT | US20190085076; SEQ ID NO: 378 | 5432 |
| CII3266 | VH | PRT | W02016201389; SEQ ID NO: 378 | 5433 |
| CII3267 | VH | PRT | US20190085076; SEQ ID NO: 374 | 5434 |
| CII3268 | VH | PRT | W02016201389; SEQ ID NO: 374 | 5435 |
| CII3269 | VH | PRT | WO2016201388; SEQ ID NO: 50 | 5436 |
| CII3270 | VH | PRT | WO2016201388; SEQ ID NO: 204 | 5437 |
| CII3271 | VH | PRT | WO2016201388; SEQ ID NO: 169 | 5438 |
| CII3272 | VH | PRT | US20190085076; SEQ ID NO: 421 | 5439 |
| CII3273 | VH | PRT | W02016201389; SEQ ID NO: 421 | 5440 |
| CII3274 | VH | PRT | US20190085076; SEQ ID NO: 387 | 5441 |
| CII3275 | VH | PRT | W02016201389; SEQ ID NO: 387 | 5442 |
| CII3276 | VH | PRT | US20190085076; SEQ ID NO: 407 | 5443 |
| CII3277 | VH | PRT | W02016201389; SEQ ID NO: 407 | 5444 |
| CII3278 | VH | PRT | US20190085076; SEQ ID NO: 383 | 5445 |
| CII3279 | VH | PRT | W02016201389; SEQ ID NO: 383 | 5446 |
| CII3280 | VH | PRT | WO2016201388; SEQ ID NO: 163 | 5447 |
| CII3281 | VH | PRT | US20190085076; SEQ ID NO: 365 | 5448 |
| CII3282 | VH | PRT | W02016201389; SEQ ID NO: 365 | 5449 |
| CII3283 | VH | PRT | US20190085076; SEQ ID NO: 409 | 5450 |
| CII3284 | VH | PRT | W02016201389; SEQ ID NO: 409 | 5451 |
| CII3285 | VH | PRT | US20190085076; SEQ ID NO: 408 | 5452 |
| CII3286 | VH | PRT | W02016201389; SEQ ID NO: 408 | 5453 |
| CII3287 | VH | PRT | WO2019028283; SEQ ID NO: 43 | 5454 |
| CII3288 | VH | PRT | WO2016201388; SEQ ID NO: 62 | 5455 |
| CII3289 | VH | PRT | WO2019028283; SEQ ID NO: 49 | 5456 |
| CII3290 | VH | PRT | WO2016201388; SEQ ID NO: 56 | 5457 |
| CII3291 | VH | PRT | WO2016201388; SEQ ID NO: 250 | 5458 |
| CII3292 | VH | PRT | WO2016201388; SEQ ID NO: 51 | 5459 |
| CII3293 | VH | PRT | WO2016201388; SEQ ID NO: 210 | 5460 |
| CII3294 | VH | PRT | WO2016201388; SEQ ID NO: 172 | 5461 |
| CII3295 | VH | PRT | WO2019028283; SEQ ID NO: 51 | 5462 |
| CII3296 | VH | PRT | WO2018089789; SEQ ID NO: 10 | 5463 |
| CII3297 | VH | PRT | US20180201692; SEQ ID NO: 2 | 5464 |
| CII3298 | VH | PRT | US20180201692; SEQ ID NO: 22 | 5465 |
| CII3299 | VH | PRT | US20180201692; SEQ ID NO: 23 | 5466 |
| CII3300 | VH | PRT | US20180201692; SEQ ID NO: 24 | 5467 |
| CII3301 | VH | PRT | US20190117769; SEQ ID NO: 6 | 5468 |
| CII3302 | VH | PRT | US20190117769; SEQ ID NO: 10 | 5469 |
| CII3303 | VH | PRT | US20190117769; SEQ ID NO: 8 | 5470 |
| CII3304 | VH | PRT | US20190135920; SEQ ID NO: 24 | 5471 |
| CII3305 | VH | PRT | US20190135920; SEQ ID NO: 19 | 5472 |
| CII3306 | VH | PRT | US20190135920; SEQ ID NO: 35 | 5473 |
| CII3307 | VH | PRT | US20190135920; SEQ ID NO: 7 | 5474 |
| CII3308 | VH | PRT | US20190135920; SEQ ID NO: 5 | 5475 |
| CII3309 | VH | PRT | US20190135920; SEQ ID NO: 1 | 5476 |
| CII3310 | VH | PRT | US20190135920; SEQ ID NO: 2 | 5477 |
| CII3311 | VH | PRT | US20190135920; SEQ ID NO: 4 | 5478 |
| CII3312 | VH | PRT | US20190135920; SEQ ID NO: 6 | 5479 |
| CII3313 | VH | PRT | US20190135920; SEQ ID NO: 23 | 5480 |
| CII3314 | VH | PRT | US20190135920; SEQ ID NO: 15 | 5481 |
| CII3315 | VH | PRT | US20190135920; SEQ ID NO: 17 | 5482 |
| CII3316 | VH | PRT | US20190135920; SEQ ID NO: 22 | 5483 |
| CII3317 | VH | PRT | US20190135920; SEQ ID NO: 20 | 5484 |
| CII3318 | VH | PRT | US20190135920; SEQ ID NO: 16 | 5485 |
| CII3319 | VH | PRT | US20190135920; SEQ ID NO: 21 | 5486 |
| CII3320 | VH | PRT | US20190135920; SEQ ID NO: 18 | 5487 |
| CII3321 | VH | PRT | US20180333503; SEQ ID NO: 26 | 5488 |
| CII3322 | VH | PRT | US20190135920; SEQ ID NO: 3 | 5489 |
| CII3323 | VH | PRT | US20190016807; SEQ ID NO: 16 | 5490 |
| CII3324 | VH | PRT | US29190016807; SEQ ID NO: 2 | 5491 |
| CII3325 | VH | PRT | US20170253653; SEQ ID NO: 4 | 5492 |
| CII3326 | VH | PRT | US20190016807; SEQ ID NO: 4 | 5493 |
| CII3327 | VH | PRT | US29190016807; SEQ ID NO: 20 | 5494 |
| CII3328 | VH | PRT | WO2018089781; SEQ ID NO: 2 | 5495 |
| CII3329 | VH | PRT | US20199016807; SEQ ID NO: 15 | 5496 |
| CII3330 | VH | PRT | US29190016807; SEQ ID NO: 1 | 5497 |
| CII3331 | VH | PRT | US20170253653; SEQ ID NO: 17 | 5498 |
| CII3332 | VH | PRT | US20170253653; SEQ ID NO: 3 | 5499 |
| CII3333 | VH | PRT | US20170253653; SEQ ID NO: 21 | 5500 |
| CII3334 | VH | PRT | US20190016807; SEQ ID NO: 10 | 5501 |
| CII3335 | VH | PRT | US20199016807; SEQ ID NO: 8 | 5502 |
| CII3336 | VH | PRT | US29190016807; SEQ ID NO: 6 | 5503 |
| CII3337 | VH | PRT | US20170253653; SEQ ID NO: 6 | 5504 |
| CII3338 | VH | PRT | US20199016807; SEQ ID NO: 3 | 5505 |
| CII3339 | VH | PRT | US20190016807; SEQ ID NO: 9 | 5506 |
| CII3340 | VH | PRT | US20170253653; SEQ ID NO: 22 | 5507 |
| CII3341 | VH | PRT | US20170253653; SEQ ID NO: 16 | 5508 |
| CII3342 | VH | PRT | US20170253653; SEQ ID NO: 2 | 5509 |
| CII3343 | VH | PRT | US20170253653; SEQ ID NO: 15 | 5510 |
| CII3344 | VH | PRT | US20199016807; SEQ ID NO: 7 | 5511 |
| CII3345 | VH | PRT | US20190016807; SEQ ID NO: 5 | 5512 |
| CII3346 | VH | PRT | US20170253653; SEQ ID NO: 5 | 5513 |
| CII3347 | VH | PRT | US20170253653; SEQ ID NO: 1 | 5514 |
| CII3348 | VH | PRT | WO2018213316; SEQ ID NO: 119 | 5515 |
| CII3349 | VH | PRT | WO2018213316; SEQ ID NO: 120 | 5516 |
| CII3350 | VH | PRT | WO2018213316; SEQ ID NO: 118 | 5517 |
| CII3351 | VH | PRT | WO2018213316; SEQ ID NO: 117 | 5518 |
| CII3352 | VH | PRT | WO2018213316; SEQ ID NO: 121 | 5519 |
| CII3353 | VH | PRT | WO2018213316; SEQ ID NO: 128 | 5520 |
| CII3354 | VH | PRT | WO2018213316; SEQ ID NO: 122 | 5521 |
| CII3355 | VH | PRT | WO2018213316; SEQ ID NO: 129 | 5522 |
| CII3356 | VH | PRT | WO2018213316; SEQ ID NO: 125 | 5523 |
| CII3357 | VH | PRT | WO2018213316; SEQ ID NO: 124 | 5524 |
| CII3358 | VH | PRT | WO2018213316; SEQ ID NO: 123 | 5525 |
| CII3359 | VH | PRT | WO2018213316; SEQ ID NO: 127 | 5526 |
| CII3360 | VH | PRT | WO2018213316; SEQ ID NO: 126 | 5527 |
| CII3361 | VH | PRT | WO2017040301; SEQ ID NO: 469 | 5528 |
| CII3362 | VH | PRT | WO2017040301; SEQ ID NO: 474 | 5529 |
| CII3363 | VH | PRT | WO2017040301; SEQ ID NO: 464 | 5530 |
| CII3364 | VH | PRT | WO2017040301; SEQ ID NO: 389 | 5531 |
| CII3365 | VH | PRT | WO2017040301; SEQ ID NO: 331 | 5532 |
| CII3366 | VH | PRT | WO2017040301; SEQ ID NO: 413 | 5533 |
| CII3367 | VH | PRT | WO2017040301; SEQ ID NO: 400 | 5534 |
| CII3368 | VH | PRT | WO2017040301; SEQ ID NO: 339 | 5535 |
| CII3369 | VH | PRT | WO2017040301; SEQ ID NO: 439 | 5536 |
| CII3370 | VH | PRT | WO2017040301; SEQ ID NO: 320 | 5537 |
| CII3371 | VH | PRT | WO2017040301; SEQ ID NO: 350 | 5538 |
| CII3372 | VH | PRT | WO2017040301; SEQ ID NO: 370 | 5539 |
| CII3373 | VH | PRT | WO2017040301; SEQ ID NO: 420 | 5540 |
| CII3374 | VH | PRT | WO2017040301; SEQ ID NO: 432 | 5541 |
| CII3375 | VH | PRT | WO2017040301; SEQ ID NO: 361 | 5542 |
| CII3376 | VH | PRT | WO2017040301; SEQ ID NO: 385 | 5543 |
| CII3377 | VH | PRT | WO2017040301; SEQ ID NO: 506 | 5544 |
| CII3378 | VH | PRT | WO2017040301; SEQ ID NO: 492 | 5545 |
| CII3379 | VH | PRT | WO2017040301; SEQ ID NO: 510 | 5546 |
| CII3380 | VH | PRT | WO2017040301; SEQ ID NO: 397 | 5547 |
| CII3381 | VH | PRT | WO2017040301; SEQ ID NO: 490 | 5548 |
| CII3382 | VH | PRT | WO2017040301; SEQ ID NO: 508 | 5549 |
| CII3383 | VH | PRT | WO2017040301; SEQ ID NO: 337 | 5550 |
| CII3384 | VH | PRT | WO2017040301; SEQ ID NO: 407 | 5551 |
| CII3385 | VH | PRT | WO2017040301; SEQ ID NO: 347 | 5552 |
| CII3386 | VH | PRT | WO2017040301; SEQ ID NO: 504 | 5553 |
| CII3387 | VH | PRT | WO2017040301; SEQ ID NO: 507 | 5554 |
| CII3388 | VH | PRT | WO2017040301; SEQ ID NO: 447 | 5555 |
| CII3389 | VH | PRT | WO2017040301; SEQ ID NO: 437 | 5556 |
| CII3390 | VH | PRT | WO2017040301; SEQ ID NO: 367 | 5557 |
| CII3391 | VH | PRT | WO2017040301; SEQ ID NO: 387 | 5558 |
| CII3392 | VH | PRT | WO2017040301; SEQ ID NO: 491 | 5559 |
| CII3393 | VH | PRT | WO2017040301; SEQ ID NO: 509 | 5560 |
| CII3394 | VH | PRT | WO2017040301; SEQ ID NO: 390 | 5561 |
| CII3395 | VH | PRT | WO2017040301; SEQ ID NO: 392 | 5562 |
| CII3396 | VH | PRT | WO2017040301; SEQ ID NO: 334 | 5563 |
| CII3397 | VH | PRT | WO2017040301; SEQ ID NO: 335 | 5564 |
| CII3398 | VH | PRT | WO2017040301; SEQ ID NO: 411 | 5565 |
| CII3399 | VH | PRT | WO2017040301; SEQ ID NO: 412 | 5566 |
| CII3400 | VH | PRT | WO2017040301; SEQ ID NO: 399 | 5567 |
| CII3401 | VH | PRT | WO2017040301; SEQ ID NO: 402 | 5568 |
| CII3402 | VH | PRT | WO2017040301; SEQ ID NO: 340 | 5569 |
| CII3403 | VH | PRT | WO2017040301; SEQ ID NO: 342 | 5570 |
| CII3404 | VH | PRT | WO2017040301; SEQ ID NO: 440 | 5571 |
| CII3405 | VH | PRT | WO2017040301; SEQ ID NO: 441 | 5572 |
| CII3406 | VH | PRT | WO2017040301; SEQ ID NO: 323 | 5573 |
| CII3407 | VH | PRT | WO2017040301; SEQ ID NO: 324 | 5574 |
| CII3408 | VH | PRT | WO2017040301; SEQ ID NO: 353 | 5575 |
| CII3409 | VH | PRT | WO2017040301; SEQ ID NO: 354 | 5576 |
| CII3410 | VH | PRT | WO2017040301; SEQ ID NO: 373 | 5577 |
| CII3411 | VH | PRT | WO2017040301; SEQ ID NO: 374 | 5578 |
| CII3412 | VH | PRT | WO2017040301; SEQ ID NO: 423 | 5579 |
| CII3413 | VH | PRT | WO2017040301; SEQ ID NO: 424 | 5580 |
| CII3414 | VH | PRT | WO2017040301; SEQ ID NO: 433 | 5581 |
| CII3415 | VH | PRT | WO2017040301; SEQ ID NO: 434 | 5582 |
| CII3416 | VH | PRT | WO2017040301; SEQ ID NO: 363 | 5583 |
| CII3417 | VH | PRT | WO2017040301; SEQ ID NO: 364 | 5584 |
| CII3418 | VH | PRT | WO2017040301; SEQ ID NO: 380 | 5585 |
| CII3419 | VH | PRT | WO2017040301; SEQ ID NO: 382 | 5586 |
| CII3420 | VH | PRT | WO2017040301; SEQ ID NO: 395 | 5587 |
| CII3421 | VH | PRT | WO2017040301; SEQ ID NO: 330 | 5588 |
| CII3422 | VH | PRT | WO2017040301; SEQ ID NO: 414 | 5589 |
| CII3423 | VH | PRT | WO2017040301; SEQ ID NO: 405 | 5590 |
| CII3424 | VH | PRT | WO2017040301; SEQ ID NO: 345 | 5591 |
| CII3425 | VH | PRT | WO2017040301; SEQ ID NO: 445 | 5592 |
| CII3426 | VH | PRT | WO2017040301; SEQ ID NO: 322 | 5593 |
| CII3427 | VH | PRT | WO2017040301; SEQ ID NO: 352 | 5594 |
| CII3428 | VH | PRT | WO2017040301; SEQ ID NO: 372 | 5595 |
| CII3429 | VH | PRT | WO2017040301; SEQ ID NO: 422 | 5596 |
| CII3430 | VH | PRT | WO2017040301; SEQ ID NO: 429 | 5597 |
| CII3431 | VH | PRT | WO2017040301; SEQ ID NO: 359 | 5598 |
| CII3432 | VH | PRT | WO2017040301; SEQ ID NO: 379 | 5599 |
| CII3433 | VH | PRT | WO2017040301; SEQ ID NO: 470 | 5600 |
| CII3434 | VH | PRT | WO2017040301; SEQ ID NO: 398 | 5601 |
| CII3435 | VH | PRT | WO2017040301; SEQ ID NO: 418 | 5602 |
| CII3436 | VH | PRT | WO2017040301; SEQ ID NO: 348 | 5603 |
| CII3437 | VH | PRT | WO2017040301; SEQ ID NO: 448 | 5604 |
| CII3438 | VH | PRT | WO2017040301; SEQ ID NO: 327 | 5605 |
| CII3439 | VH | PRT | WO2017040301; SEQ ID NO: 357 | 5606 |
| CII3440 | VH | PRT | WO2017040301; SEQ ID NO: 377 | 5607 |
| CII3441 | VH | PRT | WO2017040301; SEQ ID NO: 427 | 5608 |
| CII3442 | VH | PRT | WO2017040301; SEQ ID NO: 438 | 5609 |
| CII3443 | VH | PRT | WO2017040301; SEQ ID NO: 520 | 5610 |
| CII3444 | VH | PRT | WO2017040301; SEQ ID NO: 471 | 5611 |
| CII3445 | VH | PRT | WO2017040301; SEQ ID NO: 396 | 5612 |
| CII3446 | VH | PRT | WO2017040301; SEQ ID NO: 336 | 5613 |
| CII3447 | VH | PRT | WO2017040301; SEQ ID NO: 409 | 5614 |
| CII3448 | VH | PRT | WO2017040301; SEQ ID NO: 406 | 5615 |
| CII3449 | VH | PRT | WO2017040301; SEQ ID NO: 346 | 5616 |
| CII3450 | VH | PRT | WO2017040301; SEQ ID NO: 446 | 5617 |
| CII3451 | VH | PRT | WO2017040301; SEQ ID NO: 326 | 5618 |
| CII3452 | VH | PRT | WO2017040301; SEQ ID NO: 356 | 5619 |
| CII3453 | VH | PRT | WO2017040301; SEQ ID NO: 376 | 5620 |
| CII3454 | VH | PRT | WO2017040301; SEQ ID NO: 426 | 5621 |
| CII3455 | VH | PRT | WO2017040301; SEQ ID NO: 436 | 5622 |
| CII3456 | VH | PRT | WO2017040301; SEQ ID NO: 366 | 5623 |
| CII3457 | VH | PRT | WO2017040301; SEQ ID NO: 386 | 5624 |
| CII3458 | VH | PRT | WO2017040301; SEQ ID NO: 338 | 5625 |
| CII3459 | VH | PRT | WO2017040301; SEQ ID NO: 417 | 5626 |
| CII3460 | VH | PRT | WO2017040301; SEQ ID NO: 408 | 5627 |
| CII3461 | VH | PRT | WO2017040301; SEQ ID NO: 328 | 5628 |
| CII3462 | VH | PRT | WO2017040301; SEQ ID NO: 358 | 5629 |
| CII3463 | VH | PRT | WO2017040301; SEQ ID NO: 378 | 5630 |
| CII3464 | VH | PRT | WO2017040301; SEQ ID NO: 428 | 5631 |
| CII3465 | VH | PRT | WO2017040301; SEQ ID NO: 368 | 5632 |
| CII3466 | VH | PRT | WO2017040301; SEQ ID NO: 388 | 5633 |
| CII3467 | VH | PRT | WO2017040301; SEQ ID NO: 466 | 5634 |
| CII3468 | VH | PRT | WO2017040301; SEQ ID NO: 468 | 5635 |
| CII3469 | VH | PRT | WO2017040301; SEQ ID NO: 463 | 5636 |
| CII3470 | VH | PRT | WO2017040301; SEQ ID NO: 473 | 5637 |
| CII3471 | VH | PRT | WO2017040301; SEQ ID NO: 462 | 5638 |
| CII3472 | VH | PRT | WO2017040301; SEQ ID NO: 465 | 5639 |
| CII3473 | VH | PRT | WO2017040301; SEQ ID NO: 467 | 5640 |
| CII3474 | VH | PRT | WO2017040301; SEQ ID NO: 472 | 5641 |
| CII3475 | VH | PRT | WO2017040301; SEQ ID NO: 391 | 5642 |
| CII3476 | VH | PRT | WO2017040301; SEQ ID NO: 333 | 5643 |
| CII3477 | VH | PRT | WO2017040301; SEQ ID NO: 410 | 5644 |
| CII3478 | VH | PRT | WO2017040301; SEQ ID NO: 401 | 5645 |
| CII3479 | VH | PRT | WO2017040301; SEQ ID NO: 341 | 5646 |
| CII3480 | VH | PRT | WO2017040301; SEQ ID NO: 442 | 5647 |
| CII3481 | VH | PRT | WO2017040301; SEQ ID NO: 325 | 5648 |
| CII3482 | VH | PRT | WO2017040301; SEQ ID NO: 355 | 5649 |
| CII3483 | VH | PRT | WO2017040301; SEQ ID NO: 375 | 5650 |
| CII3484 | VH | PRT | WO2017040301; SEQ ID NO: 425 | 5651 |
| CII3485 | VH | PRT | WO2017040301; SEQ ID NO: 435 | 5652 |
| CII3486 | VH | PRT | WO2017040301; SEQ ID NO: 365 | 5653 |
| CII3487 | VH | PRT | WO2017040301; SEQ ID NO: 384 | 5654 |
| CII3488 | VH | PRT | WO2017040301; SEQ ID NO: 496 | 5655 |
| CII3489 | VH | PRT | WO2017040301; SEQ ID NO: 525 | 5656 |
| CII3490 | VH | PRT | WO2017040301; SEQ ID NO: 495 | 5657 |
| CII3491 | VH | PRT | WO2017040301; SEQ ID NO: 524 | 5658 |
| CII3492 | VH | PRT | WO2017040301; SEQ ID NO: 393 | 5659 |
| CII3493 | VH | PRT | WO2017040301; SEQ ID NO: 329 | 5660 |
| CII3494 | VH | PRT | WO2017040301; SEQ ID NO: 416 | 5661 |
| CII3495 | VH | PRT | WO2017040301; SEQ ID NO: 404 | 5662 |
| CII3496 | VH | PRT | WO2017040301; SEQ ID NO: 343 | 5663 |
| CII3497 | VH | PRT | WO2017040301; SEQ ID NO: 443 | 5664 |
| CII3498 | VH | PRT | WO2017040301; SEQ ID NO: 319 | 5665 |
| CII3499 | VH | PRT | WO2017040301; SEQ ID NO: 349 | 5666 |
| CII3500 | VH | PRT | WO2017040301; SEQ ID NO: 369 | 5667 |
| CII3501 | VH | PRT | WO2017040301; SEQ ID NO: 419 | 5668 |
| CII3502 | VH | PRT | WO2017040301; SEQ ID NO: 494 | 5669 |
| CII3503 | VH | PRT | WO2017040301; SEQ ID NO: 523 | 5670 |
| CII3504 | VH | PRT | WO2017040301; SEQ ID NO: 430 | 5671 |
| CII3505 | VH | PRT | WO2017040301; SEQ ID NO: 493 | 5672 |
| CII3506 | VH | PRT | WO2017040301; SEQ ID NO: 522 | 5673 |
| CII3507 | VH | PRT | WO2017040301; SEQ ID NO: 360 | 5674 |
| CII3508 | VH | PRT | WO2017040301; SEQ ID NO: 381 | 5675 |
| CII3509 | VH | PRT | WO2017040301; SEQ ID NO: 518 | 5676 |
| CII3510 | VH | PRT | WO2017040301; SEQ ID NO: 521 | 5677 |
| CII3511 | VH | PRT | WO2017040301; SEQ ID NO: 394 | 5678 |
| CII3512 | VH | PRT | WO2017040301; SEQ ID NO: 332 | 5679 |
| CII3513 | VH | PRT | WO2017040301; SEQ ID NO: 415 | 5680 |
| CII3514 | VH | PRT | WO2017040301; SEQ ID NO: 403 | 5681 |
| CII3515 | VH | PRT | WO2017040301; SEQ ID NO: 344 | 5682 |
| CII3516 | VH | PRT | WO2017040301; SEQ ID NO: 444 | 5683 |
| CII3517 | VH | PRT | WO2017040301; SEQ ID NO: 321 | 5684 |
| CII3518 | VH | PRT | WO2017040301; SEQ ID NO: 351 | 5685 |
| CII3519 | VH | PRT | WO2017040301; SEQ ID NO: 371 | 5686 |
| CII3520 | VH | PRT | WO2017040301; SEQ ID NO: 421 | 5687 |
| CII3521 | VH | PRT | WO2017040301; SEQ ID NO: 431 | 5688 |
| CII3522 | VH | PRT | WO2017040301; SEQ ID NO: 362 | 5689 |
| CII3523 | VH | PRT | WO2017040301; SEQ ID NO: 383 | 5690 |
| CII3524 | VH | PRT | WO2017040301; SEQ ID NO: 505 | 5691 |
| CII3525 | VH | PRT | WO2017040301; SEQ ID NO: 519 | 5692 |
| CII3526 | VH | PRT | WO2017075432; SEQ ID NO: 116 | 5693 |
| CII3527 | VH | PRT | WO2017075432; SEQ ID NO: 117 | 5694 |
| CII3528 | VH | PRT | WO2017075432; SEQ ID NO: 131 | 5695 |
| CII3529 | VH | PRT | WO2017075432; SEQ ID NO: 153 | 5696 |
| CII3530 | VH | PRT | WO2017075432; SEQ ID NO: 144 | 5697 |
| CII3531 | VH | PRT | WO2017075432; SEQ ID NO: 166 | 5698 |
| CII3532 | VH | PRT | WO2017075432; SEQ ID NO: 205 | 5699 |
| CII3533 | VH | PRT | WO2017075432; SEQ ID NO: 125 | 5700 |
| CII3534 | VH | PRT | WO2017075432; SEQ ID NO: 136 | 5701 |
| CII3535 | VH | PRT | WO2017075432; SEQ ID NO: 158 | 5702 |
| CII3536 | VH | PRT | WO2017075432; SEQ ID NO: 147 | 5703 |
| CII3537 | VH | PRT | WO2017075432; SEQ ID NO: 169 | 5704 |
| CII3538 | VH | PRT | WO2017075432; SEQ ID NO: 210 | 5705 |
| CII3539 | VH | PRT | WO2017075432; SEQ ID NO: 118 | 5706 |
| CII3540 | VH | PRT | WO2017075432; SEQ ID NO: 120 | 5707 |
| CII3541 | VH | PRT | WO2017075432; SEQ ID NO: 212 | 5708 |
| CII3542 | VH | PRT | WO2017075432; SEQ ID NO: 211 | 5709 |
| CII3543 | VH | PRT | WO2017075432; SEQ ID NO: 132 | 5710 |
| CII3544 | VH | PRT | WO2017075432; SEQ ID NO: 133 | 5711 |
| CII3545 | VH | PRT | WO2017075432; SEQ ID NO: 152 | 5712 |
| CII3546 | VH | PRT | WO2017075432; SEQ ID NO: 155 | 5713 |
| CII3547 | VH | PRT | WO2017075432; SEQ ID NO: 142 | 5714 |
| CII3548 | VH | PRT | WO2017075432; SEQ ID NO: 145 | 5715 |
| CII3549 | VH | PRT | WO2017075432; SEQ ID NO: 164 | 5716 |
| CII3550 | VH | PRT | WO2017075432; SEQ ID NO: 167 | 5717 |
| CII3551 | VH | PRT | WO2017075432; SEQ ID NO: 123 | 5718 |
| CII3552 | VH | PRT | WO2017075432; SEQ ID NO: 128 | 5719 |
| CII3553 | VH | PRT | WO2017075432; SEQ ID NO: 151 | 5720 |
| CII3554 | VH | PRT | WO2017075432; SEQ ID NO: 140 | 5721 |
| CII3555 | VH | PRT | WO2017075432; SEQ ID NO: 162 | 5722 |
| CII3556 | VH | PRT | WO2017075432; SEQ ID NO: 126 | 5723 |
| CII3557 | VH | PRT | WO2017075432; SEQ ID NO: 148 | 5724 |
| CII3558 | VH | PRT | WO2017075432; SEQ ID NO: 170 | 5725 |
| CII3559 | VH | PRT | WO2017075432; SEQ ID NO: 124 | 5726 |
| CII3560 | VH | PRT | WO2017075432; SEQ ID NO: 135 | 5727 |
| CII3561 | VH | PRT | WO2017075432; SEQ ID NO: 157 | 5728 |
| CII3562 | VH | PRT | WO2017075432; SEQ ID NO: 146 | 5729 |
| CII3563 | VH | PRT | WO2017075432; SEQ ID NO: 168 | 5730 |
| CII3564 | VH | PRT | WO2017075432; SEQ ID NO: 137 | 5731 |
| CII3565 | VH | PRT | WO2017075432; SEQ ID NO: 159 | 5732 |
| CII3566 | VH | PRT | WO2017075432; SEQ ID NO: 138 | 5733 |
| CII3567 | VH | PRT | WO2017075432; SEQ ID NO: 160 | 5734 |
| CII3568 | VH | PRT | WO2017075432; SEQ ID NO: 127 | 5735 |
| CII3569 | VH | PRT | WO2017075432; SEQ ID NO: 119 | 5736 |
| CII3570 | VH | PRT | WO2017075432; SEQ ID NO: 134 | 5737 |
| CII3571 | VH | PRT | WO2017075432; SEQ ID NO: 154 | 5738 |
| CII3572 | VH | PRT | WO2017075432; SEQ ID NO: 143 | 5739 |
| CII3573 | VH | PRT | WO2017075432; SEQ ID NO: 165 | 5740 |
| CII3574 | VH | PRT | WO2017075432; SEQ ID NO: 209 | 5741 |
| CII3575 | VH | PRT | WO2017075432; SEQ ID NO: 208 | 5742 |
| CII3576 | VH | PRT | WO2017075432; SEQ ID NO: 121 | 5743 |
| CII3577 | VH | PRT | WO2017075432; SEQ ID NO: 129 | 5744 |
| CII3578 | VH | PRT | WO2017075432; SEQ ID NO: 206 | 5745 |
| CII3579 | VH | PRT | WO2017075432; SEQ ID NO: 207 | 5746 |
| CII3580 | VH | PRT | WO2017075432; SEQ ID NO: 150 | 5747 |
| CII3581 | VH | PRT | WO2017075432; SEQ ID NO: 139 | 5748 |
| CII3582 | VH | PRT | WO2017075432; SEQ ID NO: 161 | 5749 |
| CII3583 | VH | PRT | WO2017075432; SEQ ID NO: 122 | 5750 |
| CII3584 | VH | PRT | WO2017075432; SEQ ID NO: 130 | 5751 |
| CII3585 | VH | PRT | WO2017075432; SEQ ID NO: 156 | 5752 |
| CII3586 | VH | PRT | WO2017075432; SEQ ID NO: 141 | 5753 |
| CII3587 | VH | PRT | WO2017075432; SEQ ID NO: 163 | 5754 |
| CII3588 | VH | PRT | WO2017075432; SEQ ID NO: 149 | 5755 |
| CII3589 | VH | PRT | WO2018107058; SEQ ID NO: 41 | 5756 |
| CII3590 | VH | PRT | WO2018107058; SEQ ID NO: 4 | 5757 |
| CII3591 | VH | PRT | WO2018107058; SEQ ID NO: 43 | 5758 |
| CII3592 | VH | PRT | WO2018107058; SEQ ID NO: 2 | 5759 |
| CII3593 | VH | PRT | WO2018107058; SEQ ID NO: 39 | 5760 |
| CII3594 | VH | PRT | WO2018107058; SEQ ID NO: 37 | 5761 |
| CII3595 | VH | PRT | WO2018107058; SEQ ID NO: 35 | 5762 |
| CII3596 | VH | PRT | US20190085084; SEQ ID NO: 317 | 5763 |
| CII3597 | VH | PRT | US20190085084; SEQ ID NO: 289 | 5764 |
| CII3598 | VH | PRT | US20190085084; SEQ ID NO: 372 | 5765 |
| CII3599 | VH | PRT | US20190085084; SEQ ID NO: 402 | 5766 |
| CII3600 | VH | PRT | US20190085084; SEQ ID NO: 288 | 5767 |
| CII3601 | VH | PRT | US20190085084; SEQ ID NO: 369 | 5768 |
| CII3602 | VH | PRT | US20190085084; SEQ ID NO: 287 | 5769 |
| CII3603 | VH | PRT | US20190085084; SEQ ID NO: 295 | 5770 |
| CII3604 | VH | PRT | US20190085084; SEQ ID NO: 294 | 5771 |
| CII3605 | VH | PRT | US20190085084; SEQ ID NO: 473 | 5772 |
| CII3606 | VH | PRT | US20190085084; SEQ ID NO: 475 | 5773 |
| CII3607 | VH | PRT | US20190085084; SEQ ID NO: 291 | 5774 |
| CII3608 | VH | PRT | US20190085084; SEQ ID NO: 437 | 5775 |
| CII3609 | VH | PRT | US20190085084; SEQ ID NO: 440 | 5776 |
| CII3610 | VH | PRT | US20190085084; SEQ ID NO: 426 | 5777 |
| CII3611 | VH | PRT | US20190085084; SEQ ID NO: 431 | 5778 |
| CII3612 | VH | PRT | US20190085084; SEQ ID NO: 431 | 5779 |
| CII3613 | VH | PRT | US20190085084; SEQ ID NO: 292 | 5780 |
| CII3614 | VH | PRT | US20190085084; SEQ ID NO: 454 | 5781 |
| CII3615 | VH | PRT | US20190085084; SEQ ID NO: 290 | 5782 |
| CII3616 | VH | PRT | US20190085084; SEQ ID NO: 414 | 5783 |
| CII3617 | VH | PRT | US20190085084; SEQ ID NO: 410 | 5784 |
| CII3618 | VH | PRT | US20190085084; SEQ ID NO: 284 | 5785 |
| CII3619 | VH | PRT | US20190085084; SEQ ID NO: 606 | 5786 |
| CII3620 | VH | PRT | US20190085084; SEQ ID NO: 327 | 5787 |
| CII3621 | VH | PRT | US20190085084; SEQ ID NO: 325 | 5788 |
| CII3622 | VH | PRT | US20190085084; SEQ ID NO: 320 | 5789 |
| CII3623 | VH | PRT | US20190085084; SEQ ID NO: 648 | 5790 |
| CII3624 | VH | PRT | US20190085084; SEQ ID NO: 648 | 5791 |
| CII3625 | VH | PRT | US20190085084; SEQ ID NO: 331 | 5792 |
| CII3626 | VH | PRT | US20190085084; SEQ ID NO: 342 | 5793 |
| CII3627 | VH | PRT | US20190085084; SEQ ID NO: 342 | 5794 |
| CII3628 | VH | PRT | US20190085084; SEQ ID NO: 285 | 5795 |
| CII3629 | VH | PRT | US20190085084; SEQ ID NO: 353 | 5796 |
| CII3630 | VH | PRT | US20190085084; SEQ ID NO: 351 | 5797 |
| CII3631 | VH | PRT | US20190085084; SEQ ID NO: 357 | 5798 |
| CII3632 | VH | PRT | US20190085084; SEQ ID NO: 349 | 5799 |
| CII3633 | VH | PRT | US20190085084; SEQ ID NO: 355 | 5800 |
| CII3634 | VH | PRT | US20190085084; SEQ ID NO: 310 | 5801 |
| CII3635 | VH | PRT | US20190085084; SEQ ID NO: 307 | 5802 |
| CII3636 | VH | PRT | US20190085084; SEQ ID NO: 306 | 5803 |
| CII3637 | VH | PRT | US20190085084; SEQ ID NO: 308 | 5804 |
| CII3638 | VH | PRT | US20190085084; SEQ ID NO: 309 | 5805 |
| CII3639 | VH | PRT | US20190085084; SEQ ID NO: 305 | 5806 |
| CII3640 | VH | PRT | US20190085084; SEQ ID NO: 302 | 5807 |
| CII3641 | VH | PRT | US20190085084; SEQ ID NO: 304 | 5808 |
| CII3642 | VH | PRT | US20190085084; SEQ ID NO: 303 | 5809 |
| CII3643 | VH | PRT | US20190085084; SEQ ID NO: 315 | 5810 |
| CII3644 | VH | PRT | US20190085084; SEQ ID NO: 312 | 5811 |
| CII3645 | VH | PRT | US20190085084; SEQ ID NO: 313 | 5812 |
| CII3646 | VH | PRT | US20190085084; SEQ ID NO: 311 | 5813 |
| CII3647 | VH | PRT | US20190085084; SEQ ID NO: 314 | 5814 |
| CII3648 | VH | PRT | US20190085084; SEQ ID NO: 300 | 5815 |
| CII3649 | VH | PRT | US20190085084; SEQ ID NO: 299 | 5816 |
| CII3650 | VH | PRT | US20190085084; SEQ ID NO: 297 | 5817 |
| CII3651 | VH | PRT | US20190085084; SEQ ID NO: 298 | 5818 |
| CII3652 | VH | PRT | US20190085084; SEQ ID NO: 296 | 5819 |
| CII3653 | VH | PRT | US20190085084; SEQ ID NO: 301 | 5820 |
| CII3654 | VH | PRT | WO2014028776; SEQ ID NO: 83 | 5821 |
| CII3655 | VH | PRT | US20140212413; SEQ ID NO: 5 | 5822 |
| CII3656 | VH | PRT | WO2010129469; SEQ ID NO: 2 | 5823 |
| CII3657 | VH | PRT | WO2018140121; WO2018147923; SEQ | 5824 |
| | | | ID NO: 32 | |
| CII3658 | VH | PRT | WO2010129469; SEQ ID NO: 10 | 5825 |
| CII3659 | VH | PRT | WO2018140121: WO2018147921; SEQ | 5826 |
| | | | ID NO: 7 | |
| CII3660 | VH | PRT | WO2019028292; SEQ ID NO: 120 | 5827 |
| CII3661 | VH | PRT | WO2017062672; SEQ ID NO: 429 | 5828 |
| CII3662 | VH | PRT | WO2017062672; SEQ ID NO: 481 | 5829 |
| CII3663 | VH | PRT | WO2017062672; SEQ ID NO: 399 | 5830 |
| CII3664 | VH | PRT | WO2017062672; SEQ ID NO: 637 | 5831 |
| CII3665 | VH | PRT | WO2017062672; SEQ ID NO: 554 | 5832 |
| CII3666 | VH | PRT | WO2017062672; SEQ ID NO: 573 | 5833 |
| CII3667 | VH | PRT | WO2017062672; SEQ ID NO: 442 | 5834 |
| CII3668 | VH | PRT | WO2017062672; SEQ ID NO: 494 | 5835 |
| CII3669 | VH | PRT | WO2017062672; SEQ ID NO: 649 | 5836 |
| CII3670 | VH | PRT | WO2017062672; SEQ ID NO: 484 | 5837 |
| CII3671 | VH | PRT | WO2017062672; SEQ ID NO: 432 | 5838 |
| CII3672 | VH | PRT | WO2016023019; SEQ ID NO: 319 | 5839 |
| CII3673 | VH | PRT | WO2016023019; SEQ ID NO: 244 | 5840 |
| CII3674 | VH | PRT | WO2016023019; SEQ ID NO: 317 | 5841 |
| CII3675 | VH | PRT | WO2016023019; SEQ ID NO: 312 | 5842 |
| CII3676 | VH | PRT | WO2016023019; SEQ ID NO: 288 | 5843 |
| CII3677 | VH | PRT | WO2016023019; SEQ ID NO: 280 | 5844 |
| CII3678 | VH | PRT | WO2016023019; SEQ ID NO: 282 | 5845 |
| CII3679 | VH | PRT | WO2016023019; SEQ ID NO: 290 | 5846 |
| CII3680 | VH | PRT | WO2016023019; SEQ ID NO: 294 | 5847 |
| CII3681 | VH | PRT | WO2016023019; SEQ ID NO: 304 | 5848 |
| CII3682 | VH | PRT | WO2016023019; SEQ ID NO: 308 | 5849 |
| CII3683 | VH | PRT | WO2016023019; SEQ ID NO: 298 | 5850 |
| CII3684 | VH | PRT | WO2016023019; SEQ ID NO: 286 | 5851 |
| CII3685 | VH | PRT | WO2016023019; SEQ ID NO: 284 | 5852 |
| CII3686 | VH | PRT | WO2016023019; SEQ ID NO: 242 | 5853 |
| CII3687 | VH | PRT | WO2016023019; SEQ ID NO: 315 | 5854 |
| CII3688 | VH | PRT | WO2017062672; SEQ ID NO: 401 | 5855 |
| CII3689 | VH | PRT | WO2017062672; SEQ ID NO: 474 | 5856 |
| CII3690 | VH | PRT | WO2017062672; SEQ ID NO: 514 | 5857 |
| CII3691 | VH | PRT | WO2019028292; SEQ ID NO: 85 | 5858 |
| CII3692 | VH | PRT | WO2017062672; SEQ ID NO: 525 | 5859 |
| CII3693 | VH | PRT | WO2017062672; SEQ ID NO: 535 | 5860 |
| CII3694 | VH | PRT | WO2017062672; SEQ ID NO: 547 | 5861 |
| CII3695 | VH | PRT | WO2017062672; SEQ ID NO: 467 | 5862 |
| CII3696 | VH | PRT | WO2017062672; SEQ ID NO: 671 | 5863 |
| CII3697 | VH | PRT | WO2017062672; SEQ ID NO: 567 | 5864 |
| CII3698 | VH | PRT | WO2017062672; SEQ ID NO: 423 | 5865 |
| CII3699 | VH | PRT | WO2017062672; SEQ ID NO: 661 | 5866 |
| CII3700 | VH | PRT | WO2017062672; SEQ ID NO: 413 | 5867 |
| CII3701 | VH | PRT | WO2017062672; SEQ ID NO: 505 | 5868 |
| CII3702 | VH | PRT | WO2017062672; SEQ ID NO: 451 | 5869 |
| CII3703 | VH | PRT | WO2017062672; SEQ ID NO: 511 | 5870 |
| CII3704 | VH | PRT | WO2017062672; SEQ ID NO: 409 | 5871 |
| CII3705 | VH | PRT | WO2017062672; SEQ ID NO: 731 | 5872 |
| CII3706 | VH | PRT | WO2017062672; SEQ ID NO: 733 | 5873 |
| CII3707 | VH | PRT | WO2017062672; SEQ ID NO: 636 | 5874 |
| CII3708 | VH | PRT | WO2017062672; SEQ ID NO: 552 | 5875 |
| CII3709 | VH | PRT | WO2017062672; SEQ ID NO: 572 | 5876 |
| CII3710 | VH | PRT | WO2017062672; SEQ ID NO: 440 | 5877 |
| CII3711 | VH | PRT | WO2017062672; SEQ ID NO: 492 | 5878 |
| CII3712 | VH | PRT | WO2017062672; SEQ ID NO: 647 | 5879 |
| CII3713 | VH | PRT | WO2017062672; SEQ ID NO: 482 | 5880 |
| CII3714 | VH | PRT | WO2017062672; SEQ ID NO: 430 | 5881 |
| CII3715 | VH | PRT | WO2016023019; SEQ ID NO: 374 | 5882 |
| CII3716 | VH | PRT | WO2017062672; SEQ ID NO: 472 | 5883 |
| CII3717 | VH | PRT | WO2017062672; SEQ ID NO: 519 | 5884 |
| CII3718 | VH | PRT | WO2017062672; SEQ ID NO: 529 | 5885 |
| CII3719 | VH | PRT | WO2017062672; SEQ ID NO: 539 | 5886 |
| CII3720 | VH | PRT | WO2017062672; SEQ ID NO: 549 | 5887 |
| CII3721 | VH | PRT | WO2017062672; SEQ ID NO: 469 | 5888 |
| CII3722 | VH | PRT | WO2017062672; SEQ ID NO: 677 | 5889 |
| CII3723 | VH | PRT | WO2017062672; SEQ ID NO: 569 | 5890 |
| CII3724 | VH | PRT | WO2017062672; SEQ ID NO: 427 | 5891 |
| CII3725 | VH | PRT | WO2017062672; SEQ ID NO: 666 | 5892 |
| CII3726 | VH | PRT | WO2017062672; SEQ ID NO: 417 | 5893 |
| CII3727 | VH | PRT | WO2017062672; SEQ ID NO: 509 | 5894 |
| CII3728 | VH | PRT | WO2017062672; SEQ ID NO: 457 | 5895 |
| CII3729 | VH | PRT | WO2017062672; SEQ ID NO: 639 | 5896 |
| CII3730 | VH | PRT | WO2017062672; SEQ ID NO: 640 | 5897 |
| CII3731 | VH | PRT | WO2017062672; SEQ ID NO: 553 | 5898 |
| CII3732 | VH | PRT | WO2017062672; SEQ ID NO: 574 | 5899 |
| CII3733 | VH | PRT | WO2017062672; SEQ ID NO: 441 | 5900 |
| CII3734 | VH | PRT | WO2017062672; SEQ ID NO: 493 | 5901 |
| CII3735 | VH | PRT | WO2017062672; SEQ ID NO: 648 | 5902 |
| CII3736 | VH | PRT | WO2017062672; SEQ ID NO: 651 | 5903 |
| CII3737 | VH | PRT | WO2017062672; SEQ ID NO: 483 | 5904 |
| CII3738 | VH | PRT | WO2017062672; SEQ ID NO: 431 | 5905 |
| CII3739 | VH | PRT | WO2016023019; SEQ ID NO: 380 | 5906 |
| CII3740 | VH | PRT | WO2017062672; SEQ ID NO: 402 | 5907 |
| CII3741 | VH | PRT | WO2017062672; SEQ ID NO: 476 | 5908 |
| CII3742 | VH | PRT | WO2017062672; SEQ ID NO: 516 | 5909 |
| CII3743 | VH | PRT | WO2017062672; SEQ ID NO: 526 | 5910 |
| CII3744 | VH | PRT | WO2017062672; SEQ ID NO: 537 | 5911 |
| CII3745 | VH | PRT | WO2017062672; SEQ ID NO: 545 | 5912 |
| CII3746 | VH | PRT | WO2017062672; SEQ ID NO: 465 | 5913 |
| CII3747 | VH | PRT | WO2017062672; SEQ ID NO: 674 | 5914 |
| CII3748 | VH | PRT | WO2017062672; SEQ ID NO: 675 | 5915 |
| CII3749 | VH | PRT | WO2017062672; SEQ ID NO: 565 | 5916 |
| CII3750 | VH | PRT | WO2017062672; SEQ ID NO: 425 | 5917 |
| CII3751 | VH | PRT | WO2017062672; SEQ ID NO: 663 | 5918 |
| CII3752 | VH | PRT | WO2017062672; SEQ ID NO: 664 | 5919 |
| CII3753 | VH | PRT | WO2017062672; SEQ ID NO: 415 | 5920 |
| CII3754 | VH | PRT | WO2017062672; SEQ ID NO: 506 | 5921 |
| CII3755 | VH | PRT | WO2017062672; SEQ ID NO: 454 | 5922 |
| CII3756 | VH | PRT | WO2017062672; SEQ ID NO: 635 | 5923 |
| CII3757 | VH | PRT | WO2017062672; SEQ ID NO: 460 | 5924 |
| CII3758 | VH | PRT | WO2017062672; SEQ ID NO: 439 | 5925 |
| CII3759 | VH | PRT | WO2017062672; SEQ ID NO: 551 | 5926 |
| CII3760 | VH | PRT | WO2017062672; SEQ ID NO: 643 | 5927 |
| CII3761 | VH | PRT | WO2017062672; SEQ ID NO: 558 | 5928 |
| CII3762 | VH | PRT | WO2017062672; SEQ ID NO: 578 | 5929 |
| CII3763 | VH | PRT | WO2017062672; SEQ ID NO: 445 | 5930 |
| CII3764 | VH | PRT | WO2017062672; SEQ ID NO: 498 | 5931 |
| CII3765 | VH | PRT | WO2017062672; SEQ ID NO: 654 | 5932 |
| CII3766 | VH | PRT | WO2017062672; SEQ ID NO: 488 | 5933 |
| CII3767 | VH | PRT | WO2017062672; SEQ ID NO: 436 | 5934 |
| CII3768 | VH | PRT | WO2017062672; SEQ ID NO: 406 | 5935 |
| CII3769 | VH | PRT | WO2017062672; SEQ ID NO: 478 | 5936 |
| CII3770 | VH | PRT | WO2017062672; SEQ ID NO: 512 | 5937 |
| CII3771 | VH | PRT | WO2017062672; SEQ ID NO: 522 | 5938 |
| CII3772 | VH | PRT | WO2017062672; SEQ ID NO: 532 | 5939 |
| CII3773 | VH | PRT | WO2017062672; SEQ ID NO: 542 | 5940 |
| CII3774 | VH | PRT | WO2017062672; SEQ ID NO: 462 | 5941 |
| CII3775 | VH | PRT | WO2017062672; SEQ ID NO: 670 | 5942 |
| CII3776 | VH | PRT | WO2016023019; SEQ ID NO: 260 | 5943 |
| CII3777 | VH | PRT | WO2016023019; SEQ ID NO: 349 | 5944 |
| CII3778 | VH | PRT | WO2016023019; SEQ ID NO: 264 | 5945 |
| CII3779 | VH | PRT | WO2016023019; SEQ ID NO: 341 | 5946 |
| CII3780 | VH | PRT | WO2016023019; SEQ ID NO: 355 | 5947 |
| CII3781 | VH | PRT | WO2016023019; SEQ ID NO: 353 | 5948 |
| CII3782 | VH | PRT | WO2016023019; SEQ ID NO: 369 | 5949 |
| CII3783 | VH | PRT | WO2016023019; SEQ ID NO: 258 | 5950 |
| CII3784 | VH | PRT | WO2016023019; SEQ ID NO: 262 | 5951 |
| CII3785 | VH | PRT | WO2016023019; SEQ ID NO: 410 | 5952 |
| CII3786 | VH | PRT | WO2017062672; SEQ ID NO: 563 | 5953 |
| CII3787 | VH | PRT | WO2017062672; SEQ ID NO: 421 | 5954 |
| CII3788 | VH | PRT | WO2017062672; SEQ ID NO: 660 | 5955 |
| CII3789 | VH | PRT | WO2017062672; SEQ ID NO: 411 | 5956 |
| CII3790 | VH | PRT | WO2017062672; SEQ ID NO: 503 | 5957 |
| CII3791 | VH | PRT | WO2017062672; SEQ ID NO: 453 | 5958 |
| CII3792 | VH | PRT | WO2019028292; SEQ ID NO: 65 | 5959 |
| CII3793 | VH | PRT | WO2019028292; SEQ ID NO: 69 | 5960 |
| CII3794 | VH | PRT | WO2019028292; SEQ ID NO: 67 | 5961 |
| CII3795 | VH | PRT | WO2019028292; SEQ ID NO: 55 | 5962 |
| CII3796 | VH | PRT | WO2017062672; SEQ ID NO: 471 | 5983 |
| CII3797 | VH | PRT | WO2017062672; SEQ ID NO: 817 | 5964 |
| CII3798 | VH | PRT | WO2017062672; SEQ ID NO: 646 | 5965 |
| CII3799 | VH | PRT | WO2017062672; SEQ ID NO: 491 | 5968 |
| CII3800 | VH | PRT | WO2017062672; SEQ ID NO: 560 | 5967 |
| CII3801 | VH | PRT | WO2017062672; SEQ ID NO: 580 | 5968 |
| CII3802 | VH | PRT | WO2017062672; SEQ ID NO: 490 | 5969 |
| CII3803 | VH | PRT | WO2017062672; SEQ ID NO: 438 | 5970 |
| CII3804 | VH | PRT | WO2017062672; SEQ ID NO: 407 | 5971 |
| CII3805 | VH | PRT | WO2016023019; SEQ ID NO: 271 | 5972 |
| CII3806 | VH | PRT | WO2016023019; SEQ ID NO: 256 | 5973 |
| CII3807 | VH | PRT | WO2017062672; SEQ ID NO: 480 | 5974 |
| CII3808 | VH | PRT | WO2017062672; SEQ ID NO: 550 | 5975 |
| CII3809 | VH | PRT | WO2017062672; SEQ ID NO: 470 | 5976 |
| CII3810 | VH | PRT | WO2017062672; SEQ ID NO: 678 | 5977 |
| CII3811 | VH | PRT | WO2017062672; SEQ ID NO: 428 | 5978 |
| CII3812 | VH | PRT | WO2017062672; SEQ ID NO: 667 | 5979 |
| CII3813 | VH | PRT | WO2017062672; SEQ ID NO: 418 | 5980 |
| CII3814 | VH | PRT | WO2017062672; SEQ ID NO: 824 | 5981 |
| CII3815 | VH | PRT | WO2017062672; SEQ ID NO: 644 | 5982 |
| CII3816 | VH | PRT | WO2017062672; SEQ ID NO: 559 | 5983 |
| CII3817 | VH | PRT | WO2017062672; SEQ ID NO: 579 | 5984 |
| CII3818 | VH | PRT | WO2017062672; SEQ ID NO: 447 | 5985 |
| CII3819 | VH | PRT | WO2017062672; SEQ ID NO: 499 | 5986 |
| CII3820 | VH | PRT | WO2017062672; SEQ ID NO: 655 | 5987 |
| CII3821 | VH | PRT | WO2017062672; SEQ ID NO: 489 | 5988 |
| CII3822 | VH | PRT | WO2017062672; SEQ ID NO: 437 | 5989 |
| CII3823 | VH | PRT | WO2017062672; SEQ ID NO: 400 | 5990 |
| CII3824 | VH | PRT | WO2016023019; SEQ ID NO: 274 | 5991 |
| CII3825 | VH | PRT | WO2016023019; SEQ ID NO: 395 | 5992 |
| CII3826 | VH | PRT | WO2016023019; SEQ ID NO: 277 | 5993 |
| CII3827 | VH | PRT | WO2016023019; SEQ ID NO: 367 | 5994 |
| CII3828 | VH | PRT | WO2017062672; SEQ ID NO: 479 | 5995 |
| CII3829 | VH | PRT | WO2017062672; SEQ ID NO: 518 | 5996 |
| CII3830 | VH | PRT | WO2017062672; SEQ ID NO: 528 | 5997 |
| CII3831 | VH | PRT | WO2017062672; SEQ ID NO: 538 | 5998 |
| CII3832 | VH | PRT | WO2017062672; SEQ ID NO: 548 | 5999 |
| CII3833 | VH | PRT | WO2017062672; SEQ ID NO: 468 | 6000 |
| CII3834 | VH | PRT | WO2017062672; SEQ ID NO: 676 | 6001 |
| CII3835 | VH | PRT | WO2017062672; SEQ ID NO: 568 | 6002 |
| CII3836 | VH | PRT | WO2017062672; SEQ ID NO: 426 | 6003 |
| CII3837 | VH | PRT | WO2017062672; SEQ ID NO: 665 | 6004 |
| CII3838 | VH | PRT | WO2017062672; SEQ ID NO: 416 | 6005 |
| CII3839 | VH | PRT | WO2017062672; SEQ ID NO: 508 | 6006 |
| CII3840 | VH | PRT | WO2017062672; SEQ ID NO: 456 | 6007 |
| CII3841 | VH | PRT | WO2017062672; SEQ ID NO: 645 | 6008 |
| CII3842 | VH | PRT | WO2017062672; SEQ ID NO: 448 | 6009 |
| CII3843 | VH | PRT | WO2017062672; SEQ ID NO: 500 | 6010 |
| CII3844 | VH | PRT | WO2017062672; SEQ ID NO: 656 | 6011 |
| CII3845 | VH | PRT | WO2017062672; SEQ ID NO: 408 | 6012 |
| CII3846 | VH | PRT | WO2016023019; SEQ ID NO: 268 | 6013 |
| CII3847 | VH | PRT | WO2016023019; SEQ ID NO: 386 | 6014 |
| CII3848 | VH | PRT | WO2017062672; SEQ ID NO: 520 | 6015 |
| CII3849 | VH | PRT | WO2017062672; SEQ ID NO: 530 | 6016 |
| CII3850 | VH | PRT | WO2017062672; SEQ ID NO: 540 | 6017 |
| CII3851 | VH | PRT | WO2017062672; SEQ ID NO: 570 | 6018 |
| CII3852 | VH | PRT | WO2017062672; SEQ ID NO: 510 | 6019 |
| CII3853 | VH | PRT | WO2017062672; SEQ ID NO: 458 | 6020 |
| CII3854 | VH | PRT | WO2017062672; SEQ ID NO: 732 | 6021 |
| CII3855 | VH | PRT | WO2017062672; SEQ ID NO: 818 | 6022 |
| CII3856 | VH | PRT | WO2017062672; SEQ ID NO: 531 | 6023 |
| CII3857 | VH | PRT | WO2017062672; SEQ ID NO: 449 | 6024 |
| CII3858 | VH | PRT | WO2017062672; SEQ ID NO: 461 | 6025 |
| CII3859 | VH | PRT | WO2017062672; SEQ ID NO: 820 | 6026 |
| CII3860 | VH | PRT | WO2017062672; SEQ ID NO: 419 | 6027 |
| CII3861 | VH | PRT | WO2017062672; SEQ ID NO: 541 | 6028 |
| CII3862 | VH | PRT | WO2017062672; SEQ ID NO: 501 | 6029 |
| CII3863 | VH | PRT | WO2017062672; SEQ ID NO: 668 | 6030 |
| CII3864 | VH | PRT | WO2017062672; SEQ ID NO: 521 | 6031 |
| CII3865 | VH | PRT | WO2019028292; SEQ ID NO: 119 | 6032 |
| CII3866 | VH | PRT | WO2017062672; SEQ ID NO: 657 | 6033 |
| CII3867 | VH | PRT | WO2017062672; SEQ ID NO: 825 | 6034 |
| CII3868 | VH | PRT | WO2017062672; SEQ ID NO: 823 | 6035 |
| CII3869 | VH | PRT | WO2017062672; SEQ ID NO: 819 | 6036 |
| CII3870 | VH | PRT | WO2016023019; SEQ ID NO: 388 | 6037 |
| CII3871 | VH | PRT | WO2017062672; SEQ ID NO: 561 | 6038 |
| CII3872 | VH | PRT | WO2017062672; SEQ ID NO: 638 | 6039 |
| CII3873 | VH | PRT | WO2017062672; SEQ ID NO: 555 | 6040 |
| CII3874 | VH | PRT | WO2017062672; SEQ ID NO: 575 | 6041 |
| CII3875 | VH | PRT | WO2017062672; SEQ ID NO: 443 | 6042 |
| CII3876 | VH | PRT | WO2017062672; SEQ ID NO: 495 | 6043 |
| CII3877 | VH | PRT | WO2017062672; SEQ ID NO: 650 | 6044 |
| CII3878 | VH | PRT | WO2017062672; SEQ ID NO: 485 | 6045 |
| CII3879 | VH | PRT | WO2017062672; SEQ ID NO: 433 | 6046 |
| CII3880 | VH | PRT | WO2016023019; SEQ ID NO: 360 | 6047 |
| CII3881 | VH | PRT | WO2016023019; SEQ ID NO: 296 | 6048 |
| CII3882 | VH | PRT | WO2016023019; SEQ ID NO: 393 | 6049 |
| CII3883 | VH | PRT | WO2016023019; SEQ ID NO: 300 | 6050 |
| CII3884 | VH | PRT | WO2016023019; SEQ ID NO: 302 | 6051 |
| CII3885 | VH | PRT | WO2016023019; SEQ ID NO: 376 | 6052 |
| CII3886 | VH | PRT | WO2016023019; SEQ ID NO: 292 | 6053 |
| CII3887 | VH | PRT | WO2016023019; SEQ ID NO: 391 | 6054 |
| CII3888 | VH | PRT | WO2016023019; SEQ ID NO: 378 | 6055 |
| CII3889 | VH | PRT | WO2016023019; SEQ ID NO: 310 | 6056 |
| CII3890 | VH | PRT | WO2016023019; SEQ ID NO: 306 | 6057 |
| CII3891 | VH | PRT | WO2017062672; SEQ ID NO: 403 | 6058 |
| CII3892 | VH | PRT | WO2017062672; SEQ ID NO: 473 | 6059 |
| CII3893 | VH | PRT | WO2017062672; SEQ ID NO: 517 | 6060 |
| CII3894 | VH | PRT | WO2017062672; SEQ ID NO: 527 | 6061 |
| CII3895 | VH | PRT | WO2017062672; SEQ ID NO: 536 | 6062 |
| CII3896 | VH | PRT | WO2017062672; SEQ ID NO: 546 | 6063 |
| CII3897 | VH | PRT | WO2017062672; SEQ ID NO: 466 | 6064 |
| CII3898 | VH | PRT | WO2017062672; SEQ ID NO: 673 | 6065 |
| CII3899 | VH | PRT | WO2017062672; SEQ ID NO: 566 | 6066 |
| CII3900 | VH | PRT | WO2017062672; SEQ ID NO: 422 | 6067 |
| CII3901 | VH | PRT | WO2017062672; SEQ ID NO: 662 | 6068 |
| CII3902 | VH | PRT | WO2017062672; SEQ ID NO: 414 | 6069 |
| CII3903 | VH | PRT | WO2017062672; SEQ ID NO: 507 | 6070 |
| CII3904 | VH | PRT | WO2017062672; SEQ ID NO: 455 | 6071 |
| CII3905 | VH | PRT | WO2017062672; SEQ ID NO: 847 | 6072 |
| CII3906 | VH | PRT | WO2019028292; SEQ ID NO: 75 | 6073 |
| CII3907 | VH | PRT | WO2019028292; SEQ ID NO: 77 | 6074 |
| CII3908 | VH | PRT | WO2019028292; SEQ ID NO: 88 | 6075 |
| CII3909 | VH | PRT | WO2019028292; SEQ ID NO: 87 | 6076 |
| CII3910 | VH | PRT | WO2017062672; SEQ ID NO: 642 | 6077 |
| CII3911 | VH | PRT | WO2017062672; SEQ ID NO: 557 | 6078 |
| CII3912 | VH | PRT | WO2017062672; SEQ ID NO: 577 | 6079 |
| CII3913 | VH | PRT | WO2017062672; SEQ ID NO: 446 | 6080 |
| CII3914 | VH | PRT | WO2017062672; SEQ ID NO: 496 | 6081 |
| CII3915 | VH | PRT | WO2017062672; SEQ ID NO: 653 | 6082 |
| CII3916 | VH | PRT | WO2017062672; SEQ ID NO: 486 | 6083 |
| CII3917 | VH | PRT | WO2017062672; SEQ ID NO: 435 | 6084 |
| CII3918 | VH | PRT | WO2017062672; SEQ ID NO: 405 | 6085 |
| CII3919 | VH | PRT | WO2017062672; SEQ ID NO: 475 | 6086 |
| CII3920 | VH | PRT | WO2017062672; SEQ ID NO: 513 | 6087 |
| CII3921 | VH | PRT | WO2019028292; SEQ ID NO: 71 | 6088 |
| CII3922 | VH | PRT | WO2019028292; SEQ ID NO: 59 | 6089 |
| CII3923 | VH | PRT | WO2019028292; SEQ ID NO: 79 | 6090 |
| CII3924 | VH | PRT | WO2019028292; SEQ ID NO: 80 | 6091 |
| CII3925 | VH | PRT | WO2017062672; SEQ ID NO: 846 | 6092 |
| CII3926 | VH | PRT | WO2019028292; SEQ ID NO: 74 | 6093 |
| CII3927 | VH | PRT | WO2019028292; SEQ ID NO: 78 | 6094 |
| CII3928 | VH | PRT | WO2017062672; SEQ ID NO: 523 | 6095 |
| CII3929 | VH | PRT | WO2019028292; SEQ ID NO: 81 | 6096 |
| CII3930 | VH | PRT | WO2017062672; SEQ ID NO: 845 | 6097 |
| CII3931 | VH | PRT | WO2019028292; SEQ ID NO: 73 | 6098 |
| CII3932 | VH | PRT | WO2019028292; SEQ ID NO: 89 | 6099 |
| CII3933 | VH | PRT | WO2019028292; SEQ ID NO: 90 | 6100 |
| CII3934 | VH | PRT | WO2019028292; SEQ ID NO: 56 | 6101 |
| CII3935 | VH | PRT | WO2019028292; SEQ ID NO: 58 | 6102 |
| CII3936 | VH | PRT | WO2019028292; SEQ ID NO: 86 | 6103 |
| CII3937 | VH | PRT | WO2019028292; SEQ ID NO: 57 | 6104 |
| CII3938 | VH | PRT | WO2017062672; SEQ ID NO: 533 | 6105 |
| CII3939 | VH | PRT | WO2017062672; SEQ ID NO: 544 | 6106 |
| CII3940 | VH | PRT | WO2017062672; SEQ ID NO: 464 | 6107 |
| CII3941 | VH | PRT | WO2017062672; SEQ ID NO: 669 | 6108 |
| CII3942 | VH | PRT | WO2017062672; SEQ ID NO: 562 | 6109 |
| CII3943 | VH | PRT | WO2017062672; SEQ ID NO: 420 | 6110 |
| CII3944 | VH | PRT | WO2017062672; SEQ ID NO: 658 | 6111 |
| CII3945 | VH | PRT | WO2017062672; SEQ ID NO: 410 | 6112 |
| CII3946 | VH | PRT | WO2016023019; SEQ ID NO: 357 | 6113 |
| CII3947 | VH | PRT | WO2016023019; SEQ ID NO: 362 | 6114 |
| CII3948 | VH | PRT | WO2016023019; SEQ ID NO: 343 | 6115 |
| CII3949 | VH | PRT | WO2016023019; SEQ ID NO: 335 | 6116 |
| CII3950 | VH | PRT | WO2016023019; SEQ ID NO: 337 | 6117 |
| CII3951 | VH | PRT | WO2016023019; SEQ ID NO: 372 | 6118 |
| CII3952 | VH | PRT | WO2016023019; SEQ ID NO: 345 | 6119 |
| CII3953 | VH | PRT | WO2016023019; SEQ ID NO: 339 | 6120 |
| CII3954 | VH | PRT | WO2016023019; SEQ ID NO: 347 | 6121 |
| CII3955 | VH | PRT | WO2016023019; SEQ ID NO: 266 | 6122 |
| CII3956 | VH | PRT | WO2016023019; SEQ ID NO: 351 | 6123 |
| CII3957 | VH | PRT | WO2016023019; SEQ ID NO: 250 | 6124 |
| CII3958 | VH | PRT | WO2016023019; SEQ ID NO: 412 | 6125 |
| CII3959 | VH | PRT | WO2016023019; SEQ ID NO: 252 | 6126 |
| CII3960 | VH | PRT | WO2016023019; SEQ ID NO: 365 | 6127 |
| CII3961 | VH | PRT | WO2016023019; SEQ ID NO: 322 | 6128 |
| CII3962 | VH | PRT | WO2016023019; SEQ ID NO: 331 | 6129 |
| CII3963 | VH | PRT | WO2016023019; SEQ ID NO: 324 | 6130 |
| CII3964 | VH | PRT | WO2016023019; SEQ ID NO: 326 | 6131 |
| CII3965 | VH | PRT | WO2016023019; SEQ ID NO: 254 | 6132 |
| CII3966 | VH | PRT | WO2017062672; SEQ ID NO: 502 | 6133 |
| CII3967 | VH | PRT | WO2017062672; SEQ ID NO: 450 | 6134 |
| CII3968 | VH | PRT | WO2017062672; SEQ ID NO: 641 | 6135 |
| CII3969 | VH | PRT | WO2017062672; SEQ ID NO: 556 | 6136 |
| CII3970 | VH | PRT | WO2017062672; SEQ ID NO: 576 | 6137 |
| CII3971 | VH | PRT | WO2017062672; SEQ ID NO: 444 | 6138 |
| CII3972 | VH | PRT | WO2017062672; SEQ ID NO: 497 | 6139 |
| CII3973 | VH | PRT | WO2017062672; SEQ ID NO: 652 | 6140 |
| CII3974 | VH | PRT | WO2017062672; SEQ ID NO: 487 | 6141 |
| CII3975 | VH | PRT | WO2017062672; SEQ ID NO: 434 | 6142 |
| CII3976 | VH | PRT | WO2017062672; SEQ ID NO: 404 | 6143 |
| CII3977 | VH | PRT | WO2016023019; SEQ ID NO: 384 | 6144 |
| CII3978 | VH | PRT | WO2016023019; SEQ ID NO: 246 | 6145 |
| CII3979 | VH | PRT | WO2016023019; SEQ ID NO: 382 | 6146 |
| CII3980 | VH | PRT | WO2016023019; SEQ ID NO: 248 | 6147 |
| CII3981 | VH | PRT | WO2017062672; SEQ ID NO: 477 | 6148 |
| CII3982 | VH | PRT | WO2019028292; SEQ ID NO: 31 | 6149 |
| CII3983 | VH | PRT | WO2019028292; SEQ ID NO: 32 | 6150 |
| CII3984 | VH | PRT | WO2017062672; SEQ ID NO: 515 | 6151 |
| CII3985 | VH | PRT | WO2019028292; SEQ ID NO: 33 | 6152 |
| CII3986 | VH | PRT | WO2019028292; SEQ ID NO: 34 | 6153 |
| CII3987 | VH | PRT | WO2019028292; SEQ ID NO: 35 | 6154 |
| CII3988 | VH | PRT | WO2019028292; SEQ ID NO: 64 | 6155 |
| CII3989 | VH | PRT | WO2019028292; SEQ ID NO: 63 | 6156 |
| CII3990 | VH | PRT | WO2019028292; SEQ ID NO: 54 | 6157 |
| CII3991 | VH | PRT | WO2019028292; SEQ ID NO: 36 | 6158 |
| CII3992 | VH | PRT | WO2019028292; SEQ ID NO: 37 | 6159 |
| CII3993 | VH | PRT | WO2019028292; SEQ ID NO: 38 | 6160 |
| CII3994 | VH | PRT | WO2019028292; SEQ ID NO: 39 | 6161 |
| CII3995 | VH | PRT | WO2019028292; SEQ ID NO: 40 | 6162 |
| CII3996 | VH | PRT | WO2019028292; SEQ ID NO: 41 | 6163 |
| CII3997 | VH | PRT | WO2019028292; SEQ ID NO: 42 | 6164 |
| CII3998 | VH | PRT | WO2019028292; SEQ ID NO: 43 | 6165 |
| CII3999 | VH | PRT | WO2019028292; SEQ ID NO: 44 | 6166 |
| CII4000 | VH | PRT | WO2019028292; SEQ ID NO: 45 | 6167 |
| CII4001 | VH | PRT | WO2019028292; SEQ ID NO: 68 | 6168 |
| CII4002 | VH | PRT | WO2019028292; SEQ ID NO: 66 | 6169 |
| CII4003 | VH | PRT | WO2019028292; SEQ ID NO: 61 | 6170 |
| CII4004 | VH | PRT | WO2019028292; SEQ ID NO: 91 | 6171 |
| CII4005 | VH | PRT | WO2019028292; SEQ ID NO: 46 | 6172 |
| CII4006 | VH | PRT | WO2019028292; SEQ ID NO: 60 | 6173 |
| CII4007 | VH | PRT | WO2019028292; SEQ ID NO: 47 | 6174 |
| CII4008 | VH | PRT | WO2019028292; SEQ ID NO: 48 | 6175 |
| CII4009 | VH | PRT | WO2019028292; SEQ ID NO: 62 | 6176 |
| CII4010 | VH | PRT | WO2019028292; SEQ ID NO: 49 | 6177 |
| CII4011 | VH | PRT | WO2019028292; SEQ ID NO: 50 | 6178 |
| CII4012 | VH | PRT | WO2019028292; SEQ ID NO: 51 | 6179 |
| CII4013 | VH | PRT | WO2019028292; SEQ ID NO: 52 | 6180 |
| CII4014 | VH | PRT | WO2019028292; SEQ ID NO: 70 | 6181 |
| CII4015 | VH | PRT | WO2019028292; SEQ ID NO: 53 | 6182 |
| CII4016 | VH | PRT | WO2019028292; SEQ ID NO: 84 | 6183 |
| CII4017 | VH | PRT | WO2019028292; SEQ ID NO: 83 | 6184 |
| CII4018 | VH | PRT | WO2019028292; SEQ ID NO: 82 | 6185 |
| CII4019 | VH | PRT | WO2017062672; SEQ ID NO: 524 | 6186 |
| CII4020 | VH | PRT | WO2019028292; SEQ ID NO: 72 | 6187 |
| CII4021 | VH | PRT | WO2019028292; SEQ ID NO: 27 | 6188 |
| CII4022 | VH | PRT | WO2019028292; SEQ ID NO: 29 | 6189 |
| CII4023 | VH | PRT | WO2019028292; SEQ ID NO: 28 | 6190 |
| CII4024 | VH | PRT | WO2019028292; SEQ ID NO: 30 | 6191 |
| CII4025 | VH | PRT | WO2017062672; SEQ ID NO: 534 | 6192 |
| CII4026 | VH | PRT | WO2017062672; SEQ ID NO: 543 | 6193 |
| CII4027 | VH | PRT | WO2017062672; SEQ ID NO: 463 | 6194 |
| CII4028 | VH | PRT | WO2017062672; SEQ ID NO: 672 | 6195 |
| CII4029 | VH | PRT | WO2017062672; SEQ ID NO: 564 | 6196 |
| CII4030 | VH | PRT | WO2017062672; SEQ ID NO: 424 | 6197 |
| CII4031 | VH | PRT | WO2017062672; SEQ ID NO: 659 | 6198 |
| CII4032 | VH | PRT | WO2017062672; SEQ ID NO: 412 | 6199 |
| CII4033 | VH | PRT | WO2017062672; SEQ ID NO: 504 | 6200 |
| CII4034 | VH | PRT | WO2017062672; SEQ ID NO: 452 | 6201 |
| CII4035 | VH | PRT | WO2019028292; SEQ ID NO: 76 | 6202 |
| CII4036 | VH | PRT | WO2017062672; SEQ ID NO: 571 | 6203 |
| CII4037 | VH | PRT | WO2017062672; SEQ ID NO: 459 | 6204 |
| CII4038 | VH | PRT | WO2017062672; SEQ ID NO: 822 | 6205 |
| CII4039 | VH | PRT | WO2014028776; SEQ ID NO: 75 | 6206 |
| CII4040 | VH | PRT | US20180221480; SEQ ID NO: 5 | 6207 |
| CII4041 | VH | PRT | US20180221480; SEQ ID NO: 4 | 6208 |
| CII4042 | VH | PRT | US20180221480; SEQ ID NO: 3 | 6209 |
| CII4043 | VH | PRT | US20180221480; SEQ ID NO: 2 | 6210 |
| CII4044 | VH | PRT | US20180051086; SEQ ID NO: 2 | 6211 |
| CII4045 | VH | PRT | WO2018140510; SEQ ID NO: 7 | 6212 |
| CII4046 | VL | DNA | WO2018140121; WO2018147926; SEQ | 6213 |
| | | | ID NO: 35 | |
| CII4047 | VL | DNA | WO2010129469; SEQ ID NO: 36 | 6214 |
| CII4048 | VL | DNA | US20140212413; SEQ ID NO: 2 | 6215 |
| CII4049 | VL | PRT | US20190092843; SEQ ID NO: 3 | 6216 |
| CII4050 | VL | PRT | WO2014028776; SEQ ID NO: 61 | 6217 |
| CII4051 | VL | PRT | US20180134806; SEQ ID NO: 5 | 6218 |
| CII4052 | VL | PRT | US20130243775; SEQ ID NO: 4 | 6219 |
| CII4053 | VL | PRT | WO2019020606; SEQ ID NO: 20 | 6220 |
| CII4054 | VL | PRT | WO2019020606; SEQ ID NO: 1 | 6221 |
| CII4055 | VL | PRT | WO2019020606; SEQ ID NO: 2 | 6222 |
| CII4056 | VL | PRT | WO2016201388; SEQ ID NO: 134 | 6223 |
| CII4057 | VL | PRT | WO2016201388; SEQ ID NO: 145 | 6224 |
| CII4058 | VL | PRT | WO2016201388; SEQ ID NO: 140 | 6225 |
| CII4059 | VL | PRT | WO2016201388; SEQ ID NO: 130 | 6226 |
| CII4060 | VL | PRT | WO2016201388; SEQ ID NO: 120 | 6227 |
| CII4061 | VL | PRT | WO2016201388; SEQ ID NO: 37 | 6228 |
| CII4062 | VL | PRT | WO2016201388; SEQ ID NO: 199 | 6229 |
| CII4063 | VL | PRT | US20190085076; SEQ ID NO: 341 | 6230 |
| CII4064 | VL | PRT | W02016201389; SEQ ID NO: 341 | 6231 |
| CII4065 | VL | PRT | US20190085076; SEQ ID NO: 335 | 6232 |
| CII4066 | VL | PRT | W02016201389; SEQ ID NO: 335 | 6233 |
| CII4067 | VL | PRT | US20190085076; SEQ ID NO: 353 | 6234 |
| CII4068 | VL | PRT | W02016201389; SEQ ID NO: 353 | 6235 |
| CII4069 | VL | PRT | WO2016201388; SEQ ID NO: 249 | 6236 |
| CII4070 | VL | PRT | WO2019028283; SEQ ID NO: 83 | 6237 |
| CII4071 | VL | PRT | WO2016201388; SEQ ID NO: 32 | 6238 |
| CII4072 | VL | PRT | WO2016201388; SEQ ID NO: 33 | 6239 |
| CII4073 | VL | PRT | US20190085076; SEQ ID NO: 299 | 6240 |
| CII4074 | VL | PRT | W02016201389; SEQ ID NO: 299 | 6241 |
| CII4075 | VL | PRT | US20190085076; SEQ ID NO: 314 | 6242 |
| CII4076 | VL | PRT | W02016201389; SEQ ID NO: 314 | 6243 |
| CII4077 | VL | PRT | US20190085076; SEQ ID NO: 305 | 6244 |
| CII4078 | VL | PRT | W02016201389; SEQ ID NO: 305 | 6245 |
| CII4079 | VL | PRT | US20190085076; SEQ ID NO: 345 | 6246 |
| CII4080 | VL | PRT | W02016201389; SEQ ID NO: 345 | 6247 |
| CII4081 | VL | PRT | US20190085076; SEQ ID NO: 320 | 6248 |
| CII4082 | VL | PRT | W02016201389; SEQ ID NO: 320 | 6249 |
| CII4083 | VL | PRT | US20190085076; SEQ ID NO: 342 | 6250 |
| CII4084 | VL | PRT | W02016201389; SEQ ID NO: 342 | 6251 |
| CII4085 | VL | PRT | US20190085076; SEQ ID NO: 333 | 6252 |
| CII4086 | VL | PRT | W02016201389; SEQ ID NO: 333 | 6253 |
| CII4087 | VL | PRT | US20190085076; SEQ ID NO: 321 | 6254 |
| CII4088 | VL | PRT | W02016201389; SEQ ID NO: 321 | 6255 |
| CII4089 | VL | PRT | WO2016201388; SEQ ID NO: 251 | 6256 |
| CII4090 | VL | PRT | WO2016201388; SEQ ID NO: 141 | 6257 |
| CII4091 | VL | PRT | WO2016201388; SEQ ID NO: 139 | 6258 |
| CII4092 | VL | PRT | WO2016201388; SEQ ID NO: 45 | 6259 |
| CII4093 | VL | PRT | WO2016201388; SEQ ID NO: 127 | 6260 |
| CII4094 | VL | PRT | WO2016201388; SEQ ID NO: 129 | 6261 |
| CII4095 | VL | PRT | WO2019028283; SEQ ID NO: 78 | 6262 |
| CII4096 | VL | PRT | WO2019028283; SEQ ID NO: 79 | 6263 |
| CII4097 | VL | PRT | WO2019028283; SEQ ID NO: 80 | 6264 |
| CII4098 | VL | PRT | US20190085076; SEQ ID NO: 308 | 6265 |
| CII4099 | VL | PRT | W02016201389; SEQ ID NO: 308 | 6266 |
| CII4100 | VL | PRT | US20190085076; SEQ ID NO: 300 | 6267 |
| CII4101 | VL | PRT | W02016201389; SEQ ID NO: 300 | 6268 |
| CII4102 | VL | PRT | US20190085076; SEQ ID NO: 323 | 6269 |
| CII4103 | VL | PRT | W02016201389; SEQ ID NO: 323 | 6270 |
| CII4104 | VL | PRT | US20190085076; SEQ ID NO: 327 | 6271 |
| CII4105 | VL | PRT | W02016201389; SEQ ID NO: 327 | 6272 |
| CII4106 | VL | PRT | US20190085076; SEQ ID NO: 358 | 6273 |
| CII4107 | VL | PRT | W02016201389; SEQ ID NO: 358 | 6274 |
| CII4108 | VL | PRT | US20190085076; SEQ ID NO: 362 | 6275 |
| CII4109 | VL | PRT | W02016201389; SEQ ID NO: 362 | 6276 |
| CII4110 | VL | PRT | US20190085076; SEQ ID NO: 310 | 6277 |
| CII4111 | VL | PRT | W02016201389; SEQ ID NO: 310 | 6278 |
| CII4112 | VL | PRT | US20190085076; SEQ ID NO: 347 | 6279 |
| CII4113 | VL | PRT | W02016201389; SEQ ID NO: 347 | 6280 |
| CII4114 | VL | PRT | US20190085076; SEQ ID NO: 316 | 6281 |
| CII4115 | VL | PRT | W02016201389; SEQ ID NO: 316 | 6282 |
| CII4116 | VL | PRT | US20190085076; SEQ ID NO: 340 | 6283 |
| CII4117 | VL | PRT | W02016201389; SEQ ID NO: 349 | 6284 |
| CII4118 | VL | PRT | US20190085076; SEQ ID NO: 307 | 6285 |
| CII4119 | VL | PRT | W02016201389; SEQ ID NO: 307 | 6286 |
| CII4120 | VL | PRT | US20190085076; SEQ ID NO: 344 | 6287 |
| CII4121 | VL | PRT | W02016201389; SEQ ID NO: 344 | 6288 |
| CII4122 | VL | PRT | US20190085076; SEQ ID NO: 332 | 6289 |
| CII4123 | VL | PRT | W02016201389; SEQ ID NO: 332 | 6290 |
| CII4124 | VL | PRT | US20190085076; SEQ ID NO: 315 | 6291 |
| CII4125 | VL | PRT | W02016201389; SEQ ID NO: 315 | 6292 |
| CII4126 | VL | PRT | US20190085076; SEQ ID NO: 306 | 6293 |
| CII4127 | VL | PRT | W02016201389; SEQ ID NO: 306 | 6294 |
| CII4128 | VL | PRT | US20190085076; SEQ ID NO: 311 | 6295 |
| CII4129 | VL | PRT | W02016201389; SEQ ID NO: 311 | 6296 |
| CII4130 | VL | PRT | US20190085076; SEQ ID NO: 317 | 6297 |
| CII4131 | VL | PRT | W02016201389; SEQ ID NO: 317 | 6298 |
| CII4132 | VL | PRT | US20190085076; SEQ ID NO: 312 | 6299 |
| CII4133 | VL | PRT | W02016201389; SEQ ID NO: 312 | 6300 |
| CII4134 | VL | PRT | WO2019028283; SEQ ID NO: 81 | 6301 |
| CII4135 | VL | PRT | WO2019028283; SEQ ID NO: 82 | 6302 |
| CII4136 | VL | PRT | WO2019028283; SEQ ID NO: 85 | 6303 |
| CII4137 | VL | PRT | WO2019028283; SEQ ID NO: 77 | 6304 |
| CII4138 | VL | PRT | WO2019028283; SEQ ID NO: 102 | 6305 |
| CII4139 | VL | PRT | WO2016201388; SEQ ID NO: 116 | 6306 |
| CII4140 | VL | PRT | WO2016201388; SEQ ID NO: 119 | 6307 |
| CII4141 | VL | PRT | WO2016201388; SEQ ID NO: 38 | 6308 |
| CII4142 | VL | PRT | WO2016201388; SEQ ID NO: 39 | 6309 |
| CII4143 | VL | PRT | WO2016201388; SEQ ID NO: 196 | 6310 |
| CII4144 | VL | PRT | WO2016201388; SEQ ID NO: 200 | 6311 |
| CII4145 | VL | PRT | US20190085076; SEQ ID NO: 290 | 6312 |
| CII4146 | VL | PRT | W02016201389; SEQ ID NO: 290 | 6313 |
| CII4147 | VL | PRT | US20190085076; SEQ ID NO: 318 | 6314 |
| CII4148 | VL | PRT | W02016201389; SEQ ID NO: 318 | 6315 |
| CII4149 | VL | PRT | US20190085076; SEQ ID NO: 331 | 6316 |
| CII4150 | VL | PRT | W02016201389; SEQ ID NO: 331 | 6317 |
| CII4151 | VL | PRT | US20190085076; SEQ ID NO: 330 | 6318 |
| CII4152 | VL | PRT | W02016201389; SEQ ID NO: 330 | 6319 |
| CII4153 | VL | PRT | US20190085076; SEQ ID NO: 298 | 6320 |
| CII4154 | VL | PRT | W02016201389; SEQ ID NO: 298 | 6321 |
| CII4155 | VL | PRT | US20190085076; SEQ ID NO: 302 | 6322 |
| CII4156 | VL | PRT | W02016201389; SEQ ID NO: 302 | 6323 |
| CII4157 | VL | PRT | US20190085076; SEQ ID NO: 346 | 6324 |
| CII4158 | VL | PRT | W02016201389; SEQ ID NO: 346 | 6325 |
| CII4159 | VL | PRT | US20190085076; SEQ ID NO: 328 | 6326 |
| CII4160 | VL | PRT | W02016201389; SEQ ID NO: 328 | 6327 |
| CII4161 | VL | PRT | US20190085076; SEQ ID NO: 339 | 6328 |
| CII4162 | VL | PRT | W02016201389; SEQ ID NO: 339 | 6329 |
| CII4163 | VL | PRT | US20190085076; SEQ ID NO: 303 | 6330 |
| CII4164 | VL | PRT | W02016201389; SEQ ID NO: 303 | 6331 |
| CII4165 | VL | PRT | WO2016201388; SEQ ID NO: 31 | 6332 |
| CII4166 | VL | PRT | WO2016201388; SEQ ID NO: 138 | 6333 |
| CII4167 | VL | PRT | WO2016201388; SEQ ID NO: 44 | 6334 |
| CII4168 | VL | PRT | WO2016201388; SEQ ID NO: 128 | 6335 |
| CII4169 | VL | PRT | US20190085076; SEQ ID NO: 291 | 6336 |
| CII4170 | VL | PRT | W02016201389; SEQ ID NO: 291 | 6337 |
| CII4171 | VL | PRT | US20190085076; SEQ ID NO: 354 | 6338 |
| CII4172 | VL | PRT | W02016201389; SEQ ID NO: 354 | 6339 |
| CII4173 | VL | PRT | WO2016201388; SEQ ID NO: 117 | 6340 |
| CII4174 | VL | PRT | WO2016201388; SEQ ID NO: 36 | 6341 |
| CII4175 | VL | PRT | WO2016201388; SEQ ID NO: 197 | 6342 |
| CII4176 | VL | PRT | WO2016201388; SEQ ID NO: 123 | 6343 |
| CII4177 | VL | PRT | WO2019028283; SEQ ID NO: 104 | 6344 |
| CII4178 | VL | PRT | WO2016201388; SEQ ID NO: 241 | 6345 |
| CII4179 | VL | PRT | WO2016201388; SEQ ID NO: 243 | 6346 |
| CII4180 | VL | PRT | WO2019028283; SEQ ID NO: 93 | 6347 |
| CII4181 | VL | PRT | US20190085076; SEQ ID NO: 350 | 6348 |
| CII4182 | VL | PRT | W02016201389; SEQ ID NO: 350 | 6349 |
| CII4183 | VL | PRT | WO2019028283; SEQ ID NO: 88 | 6350 |
| CII4184 | VL | PRT | WO2019028283; SEQ ID NO: 100 | 6351 |
| CII4185 | VL | PRT | WO2019028283; SEQ ID NO: 87 | 6352 |
| CII4186 | VL | PRT | WO2019028283; SEQ ID NO: 90 | 6353 |
| CII4187 | VL | PRT | WO2019028283; SEQ ID NO: 91 | 6354 |
| CII4188 | VL | PRT | WO2019028283; SEQ ID NO: 94 | 6355 |
| CII4189 | VL | PRT | WO2019028283; SEQ ID NO: 89 | 6356 |
| CII4190 | VL | PRT | WO2019028283; SEQ ID NO: 99 | 6357 |
| CII4191 | VL | PRT | WO2019028283; SEQ ID NO: 101 | 6358 |
| CII4192 | VL | PRT | WO2019028283; SEQ ID NO: 95 | 6359 |
| CII4193 | VL | PRT | WO2019028283; SEQ ID NO: 92 | 6360 |
| CII4194 | VL | PRT | WO2019028283; SEQ ID NO: 86 | 6361 |
| CII4195 | VL | PRT | WO2019028283; SEQ ID NO: 97 | 6362 |
| CII4196 | VL | PRT | WO2019028283; SEQ ID NO: 96 | 6363 |
| CII4197 | VL | PRT | WO2019028283; SEQ ID NO: 98 | 6364 |
| CII4198 | VL | PRT | WO2016201388; SEQ ID NO: 149 | 6365 |
| CII4199 | VL | PRT | US20190085076; SEQ ID NO: 349 | 6366 |
| CII4200 | VL | PRT | W02016201389; SEQ ID NO: 349 | 6367 |
| CII4201 | VL | PRT | US20190085076; SEQ ID NO: 348 | 6368 |
| CII4202 | VL | PRT | W02016201389; SEQ ID NO: 348 | 6369 |
| CII4203 | VL | PRT | US20190085076; SEQ ID NO: 351 | 6370 |
| CII4204 | VL | PRT | W02016201389; SEQ ID NO: 351 | 6371 |
| CII4205 | VL | PRT | US20190085076; SEQ ID NO: 288 | 6372 |
| CII4206 | VL | PRT | W02016201389; SEQ ID NO: 288 | 6373 |
| CII4207 | VL | PRT | US20190085076; SEQ ID NO: 352 | 6374 |
| CII4208 | VL | PRT | W02016201389; SEQ ID NO: 352 | 6375 |
| CII4209 | VL | PRT | US20190085076; SEQ ID NO: 286 | 6376 |
| CII4210 | VL | PRT | W02016201389; SEQ ID NO: 286 | 6377 |
| CII4211 | VL | PRT | US20190085076; SEQ ID NO: 360 | 6378 |
| CII4212 | VL | PRT | W02016201389; SEQ ID NO: 360 | 6379 |
| CII4213 | VL | PRT | US20190085076; SEQ ID NO: 361 | 6380 |
| CII4214 | VL | PRT | W02016201389; SEQ ID NO: 361 | 6381 |
| CII4215 | VL | PRT | US20190085076; SEQ ID NO: 359 | 6382 |
| CII4216 | VL | PRT | W02016201389; SEQ ID NO: 359 | 6383 |
| CII4217 | VL | PRT | WO2016201388; SEQ ID NO: 137 | 6384 |
| CII4218 | VL | PRT | WO2016201388; SEQ ID NO: 48 | 6385 |
| CII4219 | VL | PRT | WO2016201388; SEQ ID NO: 126 | 6386 |
| CII4220 | VL | PRT | WO2016201388; SEQ ID NO: 115 | 6387 |
| CII4221 | VL | PRT | WO2016201388; SEQ ID NO: 42 | 6388 |
| CII4222 | VL | PRT | WO2016201388; SEQ ID NO: 195 | 6389 |
| CII4223 | VL | PRT | WO2016201388; SEQ ID NO: 148 | 6390 |
| CII4224 | VL | PRT | WO2016201388; SEQ ID NO: 135 | 6391 |
| CII4225 | VL | PRT | WO2016201388; SEQ ID NO: 46 | 6392 |
| CII4226 | VL | PRT | WO2016201388; SEQ ID NO: 124 | 6393 |
| CII4227 | VL | PRT | WO2016201388; SEQ ID NO: 114 | 6394 |
| CII4228 | VL | PRT | WO2016201388; SEQ ID NO: 40 | 6395 |
| CII4229 | VL | PRT | WO2016201388; SEQ ID NO: 193 | 6396 |
| CII4230 | VL | PRT | US20190085076; SEQ ID NO: 334 | 6397 |
| CII4231 | VL | PRT | W02016201389; SEQ ID NO: 334 | 6398 |
| CII4232 | VL | PRT | US20190085076; SEQ ID NO: 326 | 6399 |
| CII4233 | VL | PRT | W02016201389; SEQ ID NO: 326 | 6400 |
| CII4234 | VL | PRT | US20190085076; SEQ ID NO: 313 | 6401 |
| CII4235 | VL | PRT | W02016201389; SEQ ID NO: 313 | 6402 |
| CII4236 | VL | PRT | WO2016201388; SEQ ID NO: 192 | 6403 |
| CII4237 | VL | PRT | WO2016201388; SEQ ID NO: 150 | 6404 |
| CII4238 | VL | PRT | WO2016201388; SEQ ID NO: 136 | 6405 |
| CII4239 | VL | PRT | WO2016201388; SEQ ID NO: 47 | 6406 |
| CII4240 | VL | PRT | WO2016201388; SEQ ID NO: 125 | 6407 |
| CII4241 | VL | PRT | WO2016201388; SEQ ID NO: 113 | 6408 |
| CII4242 | VL | PRT | WO2016201388; SEQ ID NO: 41 | 6409 |
| CII4243 | VL | PRT | WO2016201388; SEQ ID NO: 194 | 6410 |
| CII4244 | VL | PRT | WO2016201388; SEQ ID NO: 34 | 6411 |
| CII4245 | VL | PRT | WO2016201388; SEQ ID NO: 142 | 6412 |
| CII4246 | VL | PRT | WO2016201388; SEQ ID NO: 131 | 6413 |
| CII4247 | VL | PRT | US20190085076; SEQ ID NO: 309 | 6414 |
| CII4248 | VL | PRT | W02016201389; SEQ ID NO: 309 | 6415 |
| CII4249 | VL | PRT | US20190085076; SEQ ID NO: 297 | 6416 |
| CII4250 | VL | PRT | W02016201389; SEQ ID NO: 297 | 6417 |
| CII4251 | VL | PRT | US20190085076; SEQ ID NO: 325 | 6418 |
| CII4252 | VL | PRT | W02016201389; SEQ ID NO: 325 | 6419 |
| CII4253 | VL | PRT | US20190085076; SEQ ID NO: 356 | 6420 |
| CII4254 | VL | PRT | W02016201389; SEQ ID NO: 356 | 6421 |
| CII4255 | VL | PRT | US20190085076; SEQ ID NO: 357 | 6422 |
| CII4256 | VL | PRT | W02016201389; SEQ ID NO: 357 | 6423 |
| CII4257 | VL | PRT | US20190085076; SEQ ID NO: 285 | 6424 |
| CII4258 | VL | PRT | W02016201389; SEQ ID NO: 285 | 6425 |
| CII4259 | VL | PRT | US20190085076; SEQ ID NO: 301 | 6426 |
| CII4260 | VL | PRT | W02016201389; SEQ ID NO: 301 | 6427 |
| CII4261 | VL | PRT | WO2016201388; SEQ ID NO: 118 | 6428 |
| CII4262 | VL | PRT | WO2016201388; SEQ ID NO: 151 | 6429 |
| CII4263 | VL | PRT | WO2016201388; SEQ ID NO: 198 | 6430 |
| CII4264 | VL | PRT | US20190085076; SEQ ID NO: 338 | 6431 |
| CII4265 | VL | PRT | W02016201389; SEQ ID NO: 338 | 6432 |
| CII4266 | VL | PRT | WO2016201388; SEQ ID NO: 147 | 6433 |
| CII4267 | VL | PRT | WO2016201388; SEQ ID NO: 143 | 6434 |
| CII4268 | VL | PRT | WO2016201388; SEQ ID NO: 133 | 6435 |
| CII4269 | VL | PRT | US20190085076; SEQ ID NO: 324 | 6436 |
| CII4270 | VL | PRT | W02016201389; SEQ ID NO: 324 | 6437 |
| CII4271 | VL | PRT | US20190085076; SEQ ID NO: 293 | 6438 |
| CII4272 | VL | PRT | W02016201389; SEQ ID NO: 293 | 6439 |
| CII4273 | VL | PRT | US20190085076; SEQ ID NO: 296 | 6440 |
| CII4274 | VL | PRT | W02016201389; SEQ ID NO: 296 | 6441 |
| CII4275 | VL | PRT | US20190085076; SEQ ID NO: 292 | 6442 |
| CII4276 | VL | PRT | W02016201389; SEQ ID NO: 292 | 6443 |
| CII4277 | VL | PRT | US20190085076; SEQ ID NO: 304 | 6444 |
| CII4278 | VL | PRT | W02016201389; SEQ ID NO: 304 | 6445 |
| CII4279 | VL | PRT | US20190085076; SEQ ID NO: 289 | 6446 |
| CII4280 | VL | PRT | W02016201389; SEQ ID NO: 289 | 6447 |
| CII4281 | VL | PRT | US20190085076; SEQ ID NO: 329 | 6448 |
| CII4282 | VL | PRT | W02016201389; SEQ ID NO: 329 | 6449 |
| CII4283 | VL | PRT | US20190085076; SEQ ID NO: 322 | 6450 |
| CII4284 | VL | PRT | W02016201389; SEQ ID NO: 322 | 6451 |
| CII4285 | VL | PRT | US20190085076; SEQ ID NO: 343 | 6452 |
| CII4286 | VL | PRT | W02016201389; SEQ ID NO: 343 | 6453 |
| CII4287 | VL | PRT | US20190085076; SEQ ID NO: 336 | 6454 |
| CII4288 | VL | PRT | W02016201389; SEQ ID NO: 336 | 6455 |
| CII4289 | VL | PRT | WO2016201388; SEQ ID NO: 122 | 6456 |
| CII4290 | VL | PRT | WO2016201388; SEQ ID NO: 153 | 6457 |
| CII4291 | VL | PRT | WO2016201388; SEQ ID NO: 202 | 6458 |
| CII4292 | VL | PRT | US20190085076; SEQ ID NO: 355 | 6459 |
| CII4293 | VL | PRT | W02016201389; SEQ ID NO: 355 | 6460 |
| CII4294 | VL | PRT | WO2016201388; SEQ ID NO: 146 | 6461 |
| CII4295 | VL | PRT | WO2016201388; SEQ ID NO: 144 | 6462 |
| CII4296 | VL | PRT | WO2016201388; SEQ ID NO: 132 | 6463 |
| CII4297 | VL | PRT | US20190085076; SEQ ID NO: 337 | 6464 |
| CII4298 | VL | PRT | W02016201389; SEQ ID NO: 337 | 6465 |
| CII4299 | VL | PRT | US20190085076; SEQ ID NO: 287 | 6466 |
| CII4300 | VL | PRT | W02016201389; SEQ ID NO: 287 | 6467 |
| CII4301 | VL | PRT | US20190085076; SEQ ID NO: 319 | 6468 |
| CII4302 | VL | PRT | W02016201389; SEQ ID NO: 319 | 6469 |
| CII4303 | VL | PRT | US20190085076; SEQ ID NO: 294 | 6470 |
| CII4304 | VL | PRT | W02016201389; SEQ ID NO: 294 | 6471 |
| CII4305 | VL | PRT | US20190085076; SEQ ID NO: 295 | 6472 |
| CII4306 | VL | PRT | W02016201389; SEQ ID NO: 295 | 6473 |
| CII4307 | VL | PRT | WO2016201388; SEQ ID NO: 121 | 6474 |
| CII4308 | VL | PRT | WO2016201388; SEQ ID NO: 152 | 6475 |
| CII4309 | VL | PRT | WO2016201388; SEQ ID NO: 201 | 6476 |
| CII4310 | VL | PRT | WO2019028283; SEQ ID NO: 84 | 6477 |
| CII4311 | VL | PRT | WO2016201388; SEQ ID NO: 242 | 6478 |
| CII4312 | VL | PRT | WO2016201388; SEQ ID NO: 112 | 6479 |
| CII4313 | VL | PRT | WO2018089788; SEQ ID NO: 9 | 6480 |
| CII4314 | VL | PRT | US20180333503; SEQ ID NO: 5 | 6481 |
| CII4315 | VL | PRT | US20180201692; SEQ ID NO: 1 | 6482 |
| CII4316 | VL | PRT | US20180201692; SEQ ID NO: 21 | 6483 |
| CII4317 | VL | PRT | US20180201692; SEQ ID NO: 19 | 6484 |
| CII4318 | VL | PRT | US20180201692; SEQ ID NO: 20 | 6485 |
| CII4319 | VL | PRT | US20190117769; SEQ ID NO: 7 | 6486 |
| CII4320 | VL | PRT | US20190117769; SEQ ID NO: 9 | 6487 |
| CII4321 | VL | PRT | US20190117769; SEQ ID NO: 5 | 6488 |
| CII4322 | VL | PRT | US20190135920; SEQ ID NO: 34 | 6489 |
| CII4323 | VL | PRT | US20190135920; SEQ ID NO: 12 | 6490 |
| CII4324 | VL | PRT | US20190135920; SEQ ID NO: 27 | 6491 |
| CII4325 | VL | PRT | US20190135920; SEQ ID NO: 29 | 6492 |
| CII4326 | VL | PRT | US20190135920; SEQ ID NO: 14 | 6493 |
| CII4327 | VL | PRT | US20180333503; SEQ ID NO: 27 | 6494 |
| CII4328 | VL | PRT | US20190135920; SEQ ID NO: 8 | 6495 |
| CII4329 | VL | PRT | US20190135920; SEQ ID NO: 13 | 6496 |
| CII4330 | VL | PRT | US20190135920; SEQ ID NO: 26 | 6497 |
| CII4331 | VL | PRT | US20190135920; SEQ ID NO: 30 | 6498 |
| CII4332 | VL | PRT | US20190135920; SEQ ID NO: 31 | 6499 |
| CII4333 | VL | PRT | US20190135920; SEQ ID NO: 33 | 6500 |
| CII4334 | VL | PRT | US20190135920; SEQ ID NO: 10 | 6501 |
| CII4335 | VL | PRT | US20190135920; SEQ ID NO: 11 | 6502 |
| CII4336 | VL | PRT | US20190135920; SEQ ID NO: 9 | 6503 |
| CII4337 | VL | PRT | US20190135920; SEQ ID NO: 25 | 6504 |
| CII4338 | VL | PRT | US20190135920; SEQ ID NO: 32 | 6505 |
| CII4339 | VL | PRT | US20190135920; SEQ ID NO: 28 | 6506 |
| CII4340 | VL | PRT | US20190135920; SEQ ID NO: 36 | 6507 |
| CII4341 | VL | PRT | US20199016807; SEQ ID NO: 11 | 6508 |
| CII4342 | VL | PRT | US20170253653; SEQ ID NO: 19 | 6509 |
| CII4343 | VL | PRT | WO2018089780; SEQ ID NO: 1 | 6510 |
| CII4344 | VL | PRT | US20190016807; SEQ ID NO: 14 | 6511 |
| CII4345 | VL | PRT | US20170253653; SEQ ID NO: 14 | 6512 |
| CII4346 | VL | PRT | US20190016807; SEQ ID NO: 13 | 6513 |
| CII4347 | VL | PRT | US20170253653; SEQ ID NO: 13 | 6514 |
| CII4348 | VL | PRT | US20190016807; SEQ ID NO: 19 | 6515 |
| CII4349 | VL | PRT | US20170253653; SEQ ID NO: 11 | 6516 |
| CII4350 | VL | PRT | US20190016807; SEQ ID NO: 17 | 6517 |
| CII4351 | VL | PRT | US20190016807; SEQ ID NO: 18 | 6518 |
| CII4352 | VL | PRT | US20170253653; SEQ ID NO: 10 | 6519 |
| CII4353 | VL | PRT | US20170253653; SEQ ID NO: 20 | 6520 |
| CII4354 | VL | PRT | US20170253653; SEQ ID NO: 23 | 6521 |
| CII4355 | VL | PRT | US20170253653; SEQ ID NO: 18 | 6522 |
| CII4356 | VL | PRT | US20170253653; SEQ ID NO: 8 | 6523 |
| CII4357 | VL | PRT | US20170253653; SEQ ID NO: 7 | 6524 |
| CII4358 | VL | PRT | US20170253653; SEQ ID NO: 12 | 6525 |
| CII4359 | VL | PRT | US20190016807; SEQ ID NO: 12 | 6526 |
| CII4360 | VL | PRT | US20170253653; SEQ ID NO: 9 | 6527 |
| CII4361 | VL | PRT | WO2018213316; SEQ ID NO: 113 | 6528 |
| CII4362 | VL | PRT | WO2018213316; SEQ ID NO: 114 | 6529 |
| CII4363 | VL | PRT | WO2018213316; SEQ ID NO: 105 | 6530 |
| CII4364 | VL | PRT | WO2018213316; SEQ ID NO: 107 | 6531 |
| CII4365 | VL | PRT | WO2018213316; SEQ ID NO: 108 | 6532 |
| CII4366 | VL | PRT | WO2018213316; SEQ ID NO: 115 | 6533 |
| CII4367 | VL | PRT | WO2018213316; SEQ ID NO: 106 | 6534 |
| CII4368 | VL | PRT | WO2018213316; SEQ ID NO: 116 | 6535 |
| CII4369 | VL | PRT | WO2018213316; SEQ ID NO: 109 | 6536 |
| CII4370 | VL | PRT | WO2018213316; SEQ ID NO: 110 | 6537 |
| CII4371 | VL | PRT | WO2018213316; SEQ ID NO: 111 | 6538 |
| CII4372 | VL | PRT | WO2018213316; SEQ ID NO: 112 | 6539 |
| CII4373 | VL | PRT | WO2018213316; SEQ ID NO: 104 | 6540 |
| CII4374 | VL | PRT | WO2017040301; SEQ ID NO: 222 | 6541 |
| CII4375 | VL | PRT | WO2017040301; SEQ ID NO: 251 | 6542 |
| CII4376 | VL | PRT | WO2017040301; SEQ ID NO: 281 | 6543 |
| CII4377 | VL | PRT | WO2017040301; SEQ ID NO: 381 | 6544 |
| CII4378 | VL | PRT | WO2017040301; SEQ ID NO: 310 | 6545 |
| CII4379 | VL | PRT | WO2017040301; SEQ ID NO: 201 | 6546 |
| CII4380 | VL | PRT | WO2017040301; SEQ ID NO: 270 | 6547 |
| CII4381 | VL | PRT | WO2017040301; SEQ ID NO: 247 | 6548 |
| CII4382 | VL | PRT | WO2017040301; SEQ ID NO: 236 | 6549 |
| CII4383 | VL | PRT | WO2017040301; SEQ ID NO: 196 | 6550 |
| CII4384 | VL | PRT | WO2017040301; SEQ ID NO: 296 | 6551 |
| CII4385 | VL | PRT | WO2017040301; SEQ ID NO: 213 | 6552 |
| CII4386 | VL | PRT | WO2017040301; SEQ ID NO: 263 | 6553 |
| CII4387 | VL | PRT | WO2017040301; SEQ ID NO: 461 | 6554 |
| CII4388 | VL | PRT | WO2017040301; SEQ ID NO: 450 | 6555 |
| CII4389 | VL | PRT | WO2017040301; SEQ ID NO: 452 | 6556 |
| CII4390 | VL | PRT | WO2017040301; SEQ ID NO: 458 | 6557 |
| CII4391 | VL | PRT | WO2017040301; SEQ ID NO: 220 | 6558 |
| CII4392 | VL | PRT | WO2017040301; SEQ ID NO: 221 | 6559 |
| CII4393 | VL | PRT | WO2017040301; SEQ ID NO: 254 | 6560 |
| CII4394 | VL | PRT | WO2017040301; SEQ ID NO: 253 | 6561 |
| CII4395 | VL | PRT | WO2017040301; SEQ ID NO: 284 | 6562 |
| CII4396 | VL | PRT | WO2017040301; SEQ ID NO: 280 | 6563 |
| CII4397 | VL | PRT | WO2017040301; SEQ ID NO: 487 | 6564 |
| CII4398 | VL | PRT | WO2017040301; SEQ ID NO: 515 | 6565 |
| CII4399 | VL | PRT | WO2017040301; SEQ ID NO: 488 | 6566 |
| CII4400 | VL | PRT | WO2017040301; SEQ ID NO: 516 | 6567 |
| CII4401 | VL | PRT | WO2017040301; SEQ ID NO: 300 | 6568 |
| CII4402 | VL | PRT | WO2017040301; SEQ ID NO: 299 | 6569 |
| CII4403 | VL | PRT | WO2017040301; SEQ ID NO: 314 | 6570 |
| CII4404 | VL | PRT | WO2017040301; SEQ ID NO: 309 | 6571 |
| CII4405 | VL | PRT | WO2017040301; SEQ ID NO: 203 | 6572 |
| CII4406 | VL | PRT | WO2017040301; SEQ ID NO: 199 | 6573 |
| CII4407 | VL | PRT | WO2017040301; SEQ ID NO: 273 | 6574 |
| CII4408 | VL | PRT | WO2017040301; SEQ ID NO: 269 | 6575 |
| CII4409 | VL | PRT | WO2017040301; SEQ ID NO: 511 | 6576 |
| CII4410 | VL | PRT | WO2017040301; SEQ ID NO: 514 | 6577 |
| CII4411 | VL | PRT | WO2017040301; SEQ ID NO: 243 | 6578 |
| CII4412 | VL | PRT | WO2017040301; SEQ ID NO: 248 | 6579 |
| CII4413 | VL | PRT | WO2017040301; SEQ ID NO: 233 | 6580 |
| CII4414 | VL | PRT | WO2017040301; SEQ ID NO: 237 | 6581 |
| CII4415 | VL | PRT | WO2017040301; SEQ ID NO: 193 | 6582 |
| CII4416 | VL | PRT | WO2017040301; SEQ ID NO: 293 | 6583 |
| CII4417 | VL | PRT | WO2017040301; SEQ ID NO: 197 | 6584 |
| CII4418 | VL | PRT | WO2017040301; SEQ ID NO: 297 | 6585 |
| CII4419 | VL | PRT | WO2017040301; SEQ ID NO: 212 | 6586 |
| CII4420 | VL | PRT | WO2017040301; SEQ ID NO: 210 | 6587 |
| CII4421 | VL | PRT | WO2017040301; SEQ ID NO: 260 | 6588 |
| CII4422 | VL | PRT | WO2017040301; SEQ ID NO: 261 | 6589 |
| CII4423 | VL | PRT | WO2017040301; SEQ ID NO: 219 | 6590 |
| CII4424 | VL | PRT | WO2017040301; SEQ ID NO: 252 | 6591 |
| CII4425 | VL | PRT | WO2017040301; SEQ ID NO: 279 | 6592 |
| CII4426 | VL | PRT | WO2017040301; SEQ ID NO: 302 | 6593 |
| CII4427 | VL | PRT | WO2017040301; SEQ ID NO: 312 | 6594 |
| CII4428 | VL | PRT | WO2017040301; SEQ ID NO: 200 | 6595 |
| CII4429 | VL | PRT | WO2017040301; SEQ ID NO: 271 | 6596 |
| CII4430 | VL | PRT | WO2017040301; SEQ ID NO: 246 | 6597 |
| CII4431 | VL | PRT | WO2017040301; SEQ ID NO: 238 | 6598 |
| CII4432 | VL | PRT | WO2017040301; SEQ ID NO: 195 | 6599 |
| CII4433 | VL | PRT | WO2017040301; SEQ ID NO: 295 | 6600 |
| CII4434 | VL | PRT | WO2017040301; SEQ ID NO: 211 | 6601 |
| CII4435 | VL | PRT | WO2017040301; SEQ ID NO: 262 | 6602 |
| CII4436 | VL | PRT | WO2017040301; SEQ ID NO: 457 | 6603 |
| CII4437 | VL | PRT | WO2017040301; SEQ ID NO: 226 | 6604 |
| CII4438 | VL | PRT | WO2017040301; SEQ ID NO: 256 | 6605 |
| CII4439 | VL | PRT | WO2017040301; SEQ ID NO: 287 | 6606 |
| CII4440 | VL | PRT | WO2017040301; SEQ ID NO: 308 | 6607 |
| CII4441 | VL | PRT | WO2017040301; SEQ ID NO: 316 | 6608 |
| CII4442 | VL | PRT | WO2017040301; SEQ ID NO: 208 | 6609 |
| CII4443 | VL | PRT | WO2017040301; SEQ ID NO: 278 | 6610 |
| CII4444 | VL | PRT | WO2017040301; SEQ ID NO: 241 | 6611 |
| CII4445 | VL | PRT | WO2017040301; SEQ ID NO: 231 | 6612 |
| CII4446 | VL | PRT | WO2017040301; SEQ ID NO: 191 | 6613 |
| CII4447 | VL | PRT | WO2017040301; SEQ ID NO: 291 | 6614 |
| CII4448 | VL | PRT | WO2017040301; SEQ ID NO: 218 | 6615 |
| CII4449 | VL | PRT | WO2017040301; SEQ ID NO: 268 | 6616 |
| CII4450 | VL | PRT | WO2017040301; SEQ ID NO: 228 | 6617 |
| CII4451 | VL | PRT | WO2017040301; SEQ ID NO: 257 | 6618 |
| CII4452 | VL | PRT | WO2017040301; SEQ ID NO: 286 | 6619 |
| CII4453 | VL | PRT | WO2017040301; SEQ ID NO: 306 | 6620 |
| CII4454 | VL | PRT | WO2017040301; SEQ ID NO: 318 | 6621 |
| CII4455 | VL | PRT | WO2017040301; SEQ ID NO: 206 | 6622 |
| CII4456 | VL | PRT | WO2017040301; SEQ ID NO: 277 | 6623 |
| CII4457 | VL | PRT | WO2017040301; SEQ ID NO: 240 | 6624 |
| CII4458 | VL | PRT | WO2017040301; SEQ ID NO: 230 | 6625 |
| CII4459 | VL | PRT | WO2017040301; SEQ ID NO: 190 | 6626 |
| CII4460 | VL | PRT | WO2017040301; SEQ ID NO: 290 | 6627 |
| CII4461 | VL | PRT | WO2017040301; SEQ ID NO: 216 | 6628 |
| CII4462 | VL | PRT | WO2017040301; SEQ ID NO: 266 | 6629 |
| CII4463 | VL | PRT | WO2017040301; SEQ ID NO: 459 | 6630 |
| CII4464 | VL | PRT | WO2017040301; SEQ ID NO: 489 | 6631 |
| CII4465 | VL | PRT | WO2017040301; SEQ ID NO: 517 | 6632 |
| CII4466 | VL | PRT | WO2017040301; SEQ ID NO: 513 | 6633 |
| CII4467 | VL | PRT | WO2017040301; SEQ ID NO: 227 | 6634 |
| CII4468 | VL | PRT | WO2017040301; SEQ ID NO: 258 | 6635 |
| CII4469 | VL | PRT | WO2017040301; SEQ ID NO: 288 | 6636 |
| CII4470 | VL | PRT | WO2017040301; SEQ ID NO: 307 | 6637 |
| CII4471 | VL | PRT | WO2017040301; SEQ ID NO: 317 | 6638 |
| CII4472 | VL | PRT | WO2017040301; SEQ ID NO: 207 | 6639 |
| CII4473 | VL | PRT | WO2017040301; SEQ ID NO: 276 | 6640 |
| CII4474 | VL | PRT | WO2017040301; SEQ ID NO: 239 | 6641 |
| CII4475 | VL | PRT | WO2017040301; SEQ ID NO: 229 | 6642 |
| CII4476 | VL | PRT | WO2017040301; SEQ ID NO: 189 | 6643 |
| CII4477 | VL | PRT | WO2017040301; SEQ ID NO: 289 | 6644 |
| CII4478 | VL | PRT | WO2017040301; SEQ ID NO: 217 | 6645 |
| CII4479 | VL | PRT | WO2017040301; SEQ ID NO: 267 | 6646 |
| CII4480 | VL | PRT | WO2017040301; SEQ ID NO: 454 | 6647 |
| CII4481 | VL | PRT | WO2017040301; SEQ ID NO: 453 | 6648 |
| CII4482 | VL | PRT | WO2017040301; SEQ ID NO: 449 | 6649 |
| CII4483 | VL | PRT | WO2017040301; SEQ ID NO: 224 | 6650 |
| CII4484 | VL | PRT | WO2017040301; SEQ ID NO: 250 | 6651 |
| CII4485 | VL | PRT | WO2017040301; SEQ ID NO: 283 | 6652 |
| CII4486 | VL | PRT | WO2017040301; SEQ ID NO: 303 | 6653 |
| CII4487 | VL | PRT | WO2017040301; SEQ ID NO: 311 | 6654 |
| CII4488 | VL | PRT | WO2017040301; SEQ ID NO: 202 | 6655 |
| CII4489 | VL | PRT | WO2017040301; SEQ ID NO: 272 | 6656 |
| CII4490 | VL | PRT | WO2017040301; SEQ ID NO: 497 | 6657 |
| CII4491 | VL | PRT | WO2017040301; SEQ ID NO: 500 | 6658 |
| CII4492 | VL | PRT | WO2017040301; SEQ ID NO: 242 | 6659 |
| CII4493 | VL | PRT | WO2017040301; SEQ ID NO: 232 | 6660 |
| CII4494 | VL | PRT | WO2017040301; SEQ ID NO: 192 | 6661 |
| CII4495 | VL | PRT | WO2017040301; SEQ ID NO: 292 | 6662 |
| CII4496 | VL | PRT | WO2017040301; SEQ ID NO: 209 | 6663 |
| CII4497 | VL | PRT | WO2017040301; SEQ ID NO: 484 | 6664 |
| CII4498 | VL | PRT | WO2017040301; SEQ ID NO: 501 | 6665 |
| CII4499 | VL | PRT | WO2017040301; SEQ ID NO: 259 | 6666 |
| CII4500 | VL | PRT | WO2017040301; SEQ ID NO: 486 | 6667 |
| CII4501 | VL | PRT | WO2017040301; SEQ ID NO: 503 | 6668 |
| CII4502 | VL | PRT | WO2017040301; SEQ ID NO: 485 | 6669 |
| CII4503 | VL | PRT | WO2017040301; SEQ ID NO: 502 | 6670 |
| CII4504 | VL | PRT | WO2017040301; SEQ ID NO: 225 | 6671 |
| CII4505 | VL | PRT | WO2017040301; SEQ ID NO: 255 | 6672 |
| CII4506 | VL | PRT | WO2017040301; SEQ ID NO: 285 | 6673 |
| CII4507 | VL | PRT | WO2017040301; SEQ ID NO: 305 | 6674 |
| CII4508 | VL | PRT | WO2017040301; SEQ ID NO: 315 | 6675 |
| CII4509 | VL | PRT | WO2017040301; SEQ ID NO: 205 | 6676 |
| CII4510 | VL | PRT | WO2017040301; SEQ ID NO: 275 | 6677 |
| CII4511 | VL | PRT | WO2017040301; SEQ ID NO: 245 | 6678 |
| CII4512 | VL | PRT | WO2017040301; SEQ ID NO: 235 | 6679 |
| CII4513 | VL | PRT | WO2017040301; SEQ ID NO: 198 | 6680 |
| CII4514 | VL | PRT | WO2017040301; SEQ ID NO: 298 | 6681 |
| CII4515 | VL | PRT | WO2017040301; SEQ ID NO: 215 | 6682 |
| CII4516 | VL | PRT | WO2017040301; SEQ ID NO: 265 | 6683 |
| CII4517 | VL | PRT | WO2017040301; SEQ ID NO: 223 | 6684 |
| CII4518 | VL | PRT | WO2017040301; SEQ ID NO: 249 | 6685 |
| CII4519 | VL | PRT | WO2017040301; SEQ ID NO: 282 | 6686 |
| CII4520 | VL | PRT | WO2017040301; SEQ ID NO: 304 | 6687 |
| CII4521 | VL | PRT | WO2017040301; SEQ ID NO: 313 | 6688 |
| CII4522 | VL | PRT | WO2017040301; SEQ ID NO: 204 | 6689 |
| CII4523 | VL | PRT | WO2017040301; SEQ ID NO: 274 | 6690 |
| CII4524 | VL | PRT | WO2017040301; SEQ ID NO: 244 | 6691 |
| CII4525 | VL | PRT | WO2017040301; SEQ ID NO: 234 | 6692 |
| CII4526 | VL | PRT | WO2017040301; SEQ ID NO: 194 | 6693 |
| CII4527 | VL | PRT | WO2017040301; SEQ ID NO: 294 | 6694 |
| CII4528 | VL | PRT | WO2017040301; SEQ ID NO: 214 | 6695 |
| CII4529 | VL | PRT | WO2017040301; SEQ ID NO: 264 | 6696 |
| CII4530 | VL | PRT | WO2017040301; SEQ ID NO: 456 | 6697 |
| CII4531 | VL | PRT | WO2017040301; SEQ ID NO: 455 | 6698 |
| CII4532 | VL | PRT | WO2017040301; SEQ ID NO: 499 | 6699 |
| CII4533 | VL | PRT | WO2017040301; SEQ ID NO: 451 | 6700 |
| CII4534 | VL | PRT | WO2017040301; SEQ ID NO: 460 | 6701 |
| CII4535 | VL | PRT | WO2017040301; SEQ ID NO: 498 | 6702 |
| CII4536 | VL | PRT | WO2017040301; SEQ ID NO: 512 | 6703 |
| CII4537 | VL | PRT | WO2017075432; SEQ ID NO: 94 | 6704 |
| CII4538 | VL | PRT | WO2017075432; SEQ ID NO: 96 | 6705 |
| CII4539 | VL | PRT | WO2017075432; SEQ ID NO: 78 | 6706 |
| CII4540 | VL | PRT | WO2017075432; SEQ ID NO: 110 | 6707 |
| CII4541 | VL | PRT | WO2017075432; SEQ ID NO: 89 | 6708 |
| CII4542 | VL | PRT | WO2017075432; SEQ ID NO: 62 | 6709 |
| CII4543 | VL | PRT | WO2017075432; SEQ ID NO: 204 | 6710 |
| CII4544 | VL | PRT | WO2017075432; SEQ ID NO: 203 | 6711 |
| CII4545 | VL | PRT | WO2017075432; SEQ ID NO: 61 | 6712 |
| CII4546 | VL | PRT | WO2017075432; SEQ ID NO: 95 | 6713 |
| CII4547 | VL | PRT | WO2017075432; SEQ ID NO: 97 | 6714 |
| CII4548 | VL | PRT | WO2017075432; SEQ ID NO: 79 | 6715 |
| CII4549 | VL | PRT | WO2017075432; SEQ ID NO: 77 | 6716 |
| CII4550 | VL | PRT | WO2017075432; SEQ ID NO: 111 | 6717 |
| CII4551 | VL | PRT | WO2017075432; SEQ ID NO: 90 | 6718 |
| CII4552 | VL | PRT | WO2017075432; SEQ ID NO: 112 | 6719 |
| CII4553 | VL | PRT | WO2017075432; SEQ ID NO: 88 | 6720 |
| CII4554 | VL | PRT | WO2017075432; SEQ ID NO: 202 | 6721 |
| CII4555 | VL | PRT | WO2017075432; SEQ ID NO: 65 | 6722 |
| CII4556 | VL | PRT | WO2017075432; SEQ ID NO: 63 | 6723 |
| CII4557 | VL | PRT | WO2017075432; SEQ ID NO: 99 | 6724 |
| CII4558 | VL | PRT | WO2017075432; SEQ ID NO: 76 | 6725 |
| CII4559 | VL | PRT | WO2017075432; SEQ ID NO: 109 | 6726 |
| CII4560 | VL | PRT | WO2017075432; SEQ ID NO: 87 | 6727 |
| CII4561 | VL | PRT | WO2017075432; SEQ ID NO: 64 | 6728 |
| CII4562 | VL | PRT | WO2017075432; SEQ ID NO: 201 | 6729 |
| CII4563 | VL | PRT | WO2017075432; SEQ ID NO: 103 | 6730 |
| CII4564 | VL | PRT | WO2017075432; SEQ ID NO: 75 | 6731 |
| CII4565 | VL | PRT | WO2017075432; SEQ ID NO: 108 | 6732 |
| CII4566 | VL | PRT | WO2017075432; SEQ ID NO: 86 | 6733 |
| CII4567 | VL | PRT | WO2017075432; SEQ ID NO: 70 | 6734 |
| CII4568 | VL | PRT | WO2017075432; SEQ ID NO: 102 | 6735 |
| CII4569 | VL | PRT | WO2017075432; SEQ ID NO: 74 | 6736 |
| CII4570 | VL | PRT | WO2017075432; SEQ ID NO: 107 | 6737 |
| CII4571 | VL | PRT | WO2017075432; SEQ ID NO: 85 | 6738 |
| CII4572 | VL | PRT | WO2017075432; SEQ ID NO: 71 | 6739 |
| CII4573 | VL | PRT | WO2017075432; SEQ ID NO: 104 | 6740 |
| CII4574 | VL | PRT | WO2017075432; SEQ ID NO: 73 | 6741 |
| CII4575 | VL | PRT | WO2017075432; SEQ ID NO: 106 | 6742 |
| CII4576 | VL | PRT | WO2017075432; SEQ ID NO: 84 | 6743 |
| CII4577 | VL | PRT | WO2017075432; SEQ ID NO: 69 | 6744 |
| CII4578 | VL | PRT | WO2017075432; SEQ ID NO: 198 | 6745 |
| CII4579 | VL | PRT | WO2017075432; SEQ ID NO: 200 | 6746 |
| CII4580 | VL | PRT | WO2017075432; SEQ ID NO: 199 | 6747 |
| CII4581 | VL | PRT | WO2017075432; SEQ ID NO: 197 | 6748 |
| CII4582 | VL | PRT | WO2017075432; SEQ ID NO: 72 | 6749 |
| CII4583 | VL | PRT | WO2017075432; SEQ ID NO: 98 | 6750 |
| CII4584 | VL | PRT | WO2017075432; SEQ ID NO: 80 | 6751 |
| CII4585 | VL | PRT | WO2017075432; SEQ ID NO: 113 | 6752 |
| CII4586 | VL | PRT | WO2017075432; SEQ ID NO: 91 | 6753 |
| CII4587 | VL | PRT | WO2017075432; SEQ ID NO: 67 | 6754 |
| CII4588 | VL | PRT | WO2017075432; SEQ ID NO: 101 | 6755 |
| CII4589 | VL | PRT | WO2017075432; SEQ ID NO: 82 | 6756 |
| CII4590 | VL | PRT | WO2017075432; SEQ ID NO: 115 | 6757 |
| CII4591 | VL | PRT | WO2017075432; SEQ ID NO: 93 | 6758 |
| CII4592 | VL | PRT | WO2017075432; SEQ ID NO: 68 | 6759 |
| CII4593 | VL | PRT | WO2017075432; SEQ ID NO: 100 | 6760 |
| CII4594 | VL | PRT | WO2017075432; SEQ ID NO: 81 | 6761 |
| CII4595 | VL | PRT | WO2017075432; SEQ ID NO: 114 | 6762 |
| CII4596 | VL | PRT | WO2017075432; SEQ ID NO: 92 | 6763 |
| CII4597 | VL | PRT | WO2017075432; SEQ ID NO: 66 | 6764 |
| CII4598 | VL | PRT | WO2017075432; SEQ ID NO: 83 | 6765 |
| CII4599 | VL | PRT | WO2017075432; SEQ ID NO: 105 | 6766 |
| CII4600 | VL | PRT | WO2018107058; SEQ ID NO: 44 | 6767 |
| CII4601 | VL | PRT | WO2018107058; SEQ ID NO: 3 | 6768 |
| CII4602 | VL | PRT | WO2018107058; SEQ ID NO: 36 | 6769 |
| CII4603 | VL | PRT | WO2018107058; SEQ ID NO: 42 | 6770 |
| CII4604 | VL | PRT | WO2018107058; SEQ ID NO: 38 | 6771 |
| CII4605 | VL | PRT | WO2018107058; SEQ ID NO: 40 | 6772 |
| CII4606 | VL | PRT | WO2018107058; SEQ ID NO: 5 | 6773 |
| CII4607 | VL | PRT | US20190085084; SEQ ID NO: 268 | 6774 |
| CII4608 | VL | PRT | US20190085084; SEQ ID NO: 258 | 6775 |
| CII4609 | VL | PRT | US20190085084; SEQ ID NO: 354 | 6776 |
| CII4610 | VL | PRT | US20190085084; SEQ ID NO: 319 | 6777 |
| CII4611 | VL | PRT | US20190085084; SEQ ID NO: 319 | 6778 |
| CII4612 | VL | PRT | US20190085084; SEQ ID NO: 341 | 6779 |
| CII4613 | VL | PRT | US20190085084; SEQ ID NO: 350 | 6780 |
| CII4614 | VL | PRT | US20190085084; SEQ ID NO: 263 | 6781 |
| CII4615 | VL | PRT | US20190085084; SEQ ID NO: 259 | 6782 |
| CII4616 | VL | PRT | US20190085084; SEQ ID NO: 356 | 6783 |
| CII4617 | VL | PRT | US20190085084; SEQ ID NO: 356 | 6784 |
| CII4618 | VL | PRT | US20190085084; SEQ ID NO: 267 | 6785 |
| CII4619 | VL | PRT | US20190085084; SEQ ID NO: 261 | 6786 |
| CII4620 | VL | PRT | US20190085084; SEQ ID NO: 371 | 6787 |
| CII4621 | VL | PRT | US20190085084; SEQ ID NO: 430 | 6788 |
| CII4622 | VL | PRT | US20190085084; SEQ ID NO: 430 | 6789 |
| CII4623 | VL | PRT | US20190085084; SEQ ID NO: 370 | 6790 |
| CII4624 | VL | PRT | US20190085084; SEQ ID NO: 324 | 6791 |
| CII4625 | VL | PRT | US20190085084; SEQ ID NO: 264 | 6792 |
| CII4626 | VL | PRT | US20190085084; SEQ ID NO: 413 | 6793 |
| CII4627 | VL | PRT | US20190085084; SEQ ID NO: 348 | 6794 |
| CII4628 | VL | PRT | US20190085084; SEQ ID NO: 262 | 6795 |
| CII4629 | VL | PRT | US20190085084; SEQ ID NO: 257 | 6796 |
| CII4630 | VL | PRT | US20190085084; SEQ ID NO: 425 | 6797 |
| CII4631 | VL | PRT | US20190085084; SEQ ID NO: 472 | 6798 |
| CII4632 | VL | PRT | US20190085084; SEQ ID NO: 472 | 6799 |
| CII4633 | VL | PRT | US20190085084; SEQ ID NO: 266 | 6800 |
| CII4634 | VL | PRT | US20190085084; SEQ ID NO: 260 | 6801 |
| CII4635 | VL | PRT | US20190085084; SEQ ID NO: 343 | 6802 |
| CII4636 | VL | PRT | US20190085084; SEQ ID NO: 343 | 6803 |
| CII4637 | VL | PRT | US20190085084; SEQ ID NO: 265 | 6804 |
| CII4638 | VL | PRT | US20190085084; SEQ ID NO: 352 | 6805 |
| CII4639 | VL | PRT | US20190085084; SEQ ID NO: 283 | 6806 |
| CII4640 | VL | PRT | US20190085084; SEQ ID NO: 281 | 6807 |
| CII4641 | VL | PRT | US20190085084; SEQ ID NO: 279 | 6808 |
| CII4642 | VL | PRT | US20190085084; SEQ ID NO: 276 | 6809 |
| CII4643 | VL | PRT | US20190085084; SEQ ID NO: 280 | 6810 |
| CII4644 | VL | PRT | US20190085084; SEQ ID NO: 282 | 6811 |
| CII4645 | VL | PRT | US20190085084; SEQ ID NO: 277 | 6812 |
| CII4646 | VL | PRT | US20190085084; SEQ ID NO: 278 | 6813 |
| CII4647 | VL | PRT | US20190085084; SEQ ID NO: 272 | 6814 |
| CII4648 | VL | PRT | US20190085084; SEQ ID NO: 271 | 6815 |
| CII4649 | VL | PRT | US20190085084; SEQ ID NO: 275 | 6816 |
| CII4650 | VL | PRT | US20190085084; SEQ ID NO: 274 | 6817 |
| CII4651 | VL | PRT | US20190085084; SEQ ID NO: 270 | 6818 |
| CII4652 | VL | PRT | US20190085084; SEQ ID NO: 269 | 6819 |
| CII4653 | VL | PRT | US20190085084; SEQ ID NO: 273 | 6820 |
| CII4654 | VL | PRT | WO2014028776; SEQ ID NO: 82 | 6821 |
| CII4655 | VL | PRT | US20140212413; SEQ ID NO: 3 | 6822 |
| CII4656 | VL | PRT | WO2010129469; SEQ ID NO: 9 | 6823 |
| CII4657 | VL | PRT | WO2010129469; SEQ ID NO: 1 | 6824 |
| CII4658 | VL | PRT | WO2018140121; WO2018147922; SEQ | 6825 |
| | | | ID NO: 8 | |
| CII4659 | VL | PRT | WO2018140121; WO2018147924; SEQ | 6826 |
| | | | ID NO: 33 | |
| CII4660 | VL | PRT | WO2017062672; SEQ ID NO: 357 | 6827 |
| CII4661 | VL | PRT | WO2017062672; SEQ ID NO: 256 | 6828 |
| CII4662 | VL | PRT | WO2017062672; SEQ ID NO: 385 | 6829 |
| CII4663 | VL | PRT | WO2017062672; SEQ ID NO: 391 | 6830 |
| CII4664 | VL | PRT | WO2017062672; SEQ ID NO: 246 | 6831 |
| CII4665 | VL | PRT | WO2017062672; SEQ ID NO: 304 | 6832 |
| CII4666 | VL | PRT | WO2017062672; SEQ ID NO: 234 | 6833 |
| CII4667 | VL | PRT | WO2017062672; SEQ ID NO: 617 | 6834 |
| CII4668 | VL | PRT | WO2017062672; SEQ ID NO: 608 | 6835 |
| CII4669 | VL | PRT | WO2017062672; SEQ ID NO: 684 | 6836 |
| CII4670 | VL | PRT | WO2017062672; SEQ ID NO: 221 | 6837 |
| CII4671 | VL | PRT | WO2017062672; SEQ ID NO: 312 | 6838 |
| CII4672 | VL | PRT | WO2017062672; SEQ ID NO: 350 | 6839 |
| CII4673 | VL | PRT | WO2017062672; SEQ ID NO: 283 | 6840 |
| CII4674 | VL | PRT | WO2017062672; SEQ ID NO: 632 | 6841 |
| CII4675 | VL | PRT | WO2017062672; SEQ ID NO: 342 | 6842 |
| CII4676 | VL | PRT | WO2017062672; SEQ ID NO: 373 | 6843 |
| CII4677 | VL | PRT | WO2017062672; SEQ ID NO: 292 | 6844 |
| CII4678 | VL | PRT | WO2017062672; SEQ ID NO: 325 | 6845 |
| CII4679 | VL | PRT | WO2017062672; SEQ ID NO: 268 | 6846 |
| CII4680 | VL | PRT | WO2016023019; SEQ ID NO: 297 | 6847 |
| CII4681 | VL | PRT | WO2016023019; SEQ ID NO: 392 | 6848 |
| CII4682 | VL | PRT | WO2017062672; SEQ ID NO: 814 | 6849 |
| CII4683 | VL | PRT | WO2017062672; SEQ ID NO: 322 | 6850 |
| CII4684 | VL | PRT | WO2017062672; SEQ ID NO: 356 | 6851 |
| CII4685 | VL | PRT | WO2017062672; SEQ ID NO: 358 | 6852 |
| CII4686 | VL | PRT | WO2017062672; SEQ ID NO: 259 | 6853 |
| CII4687 | VL | PRT | WO2017062672; SEQ ID NO: 258 | 6854 |
| CII4688 | VL | PRT | WO2017062672; SEQ ID NO: 380 | 6855 |
| CII4689 | VL | PRT | WO2017062672; SEQ ID NO: 386 | 6856 |
| CII4690 | VL | PRT | WO2017062672; SEQ ID NO: 394 | 6857 |
| CII4691 | VL | PRT | WO2017062672; SEQ ID NO: 396 | 6858 |
| CII4692 | VL | PRT | WO2017062672; SEQ ID NO: 248 | 6859 |
| CII4693 | VL | PRT | WO2017062672; SEQ ID NO: 247 | 6860 |
| CII4694 | VL | PRT | WO2017062672; SEQ ID NO: 308 | 6861 |
| CII4695 | VL | PRT | WO2017062672; SEQ ID NO: 306 | 6862 |
| CII4696 | VL | PRT | WO2017062672; SEQ ID NO: 237 | 6863 |
| CII4697 | VL | PRT | WO2017062672; SEQ ID NO: 238 | 6864 |
| CII4698 | VL | PRT | WO2017062672; SEQ ID NO: 620 | 6865 |
| CII4699 | VL | PRT | WO2017062672; SEQ ID NO: 621 | 6866 |
| CII4700 | VL | PRT | WO2017062672; SEQ ID NO: 611 | 6867 |
| CII4701 | VL | PRT | WO2017062672; SEQ ID NO: 686 | 6868 |
| CII4702 | VL | PRT | WO2017062672; SEQ ID NO: 610 | 6869 |
| CII4703 | VL | PRT | WO2017062672; SEQ ID NO: 688 | 6870 |
| CII4704 | VL | PRT | WO2016023019; SEQ ID NO: 247 | 6871 |
| CII4705 | VL | PRT | WO2016023019; SEQ ID NO: 270 | 6872 |
| CII4706 | VL | PRT | WO2016023019; SEQ ID NO: 273 | 6873 |
| CII4707 | VL | PRT | WO2016023019; SEQ ID NO: 275 | 6874 |
| CII4708 | VL | PRT | WO2016023019; SEQ ID NO: 272 | 6875 |
| CII4709 | VL | PRT | WO2016023019; SEQ ID NO: 344 | 6876 |
| CII4710 | VL | PRT | WO2016023019; SEQ ID NO: 269 | 6877 |
| CII4711 | VL | PRT | WO2016023019; SEQ ID NO: 276 | 6878 |
| CII4712 | VL | PRT | WO2016023019; SEQ ID NO: 368 | 6879 |
| CII4713 | VL | PRT | WO2017062672; SEQ ID NO: 225 | 6880 |
| CII4714 | VL | PRT | WO2017062672; SEQ ID NO: 224 | 6881 |
| CII4715 | VL | PRT | WO2017062672; SEQ ID NO: 317 | 6882 |
| CII4716 | VL | PRT | WO2017062672; SEQ ID NO: 316 | 6883 |
| CII4717 | VL | PRT | WO2016023019; SEQ ID NO: 334 | 6884 |
| CII4718 | VL | PRT | WO2016023019; SEQ ID NO: 320 | 6885 |
| CII4719 | VL | PRT | WO2016023019; SEQ ID NO: 283 | 6886 |
| CII4720 | VL | PRT | WO2016023019; SEQ ID NO: 265 | 6887 |
| CII4721 | VL | PRT | WO2016023019; SEQ ID NO: 332 | 6888 |
| CII4722 | VL | PRT | WO2016023019; SEQ ID NO: 314 | 6889 |
| CII4723 | VL | PRT | WO2016023019; SEQ ID NO: 305 | 6890 |
| CII4724 | VL | PRT | WO2016023019; SEQ ID NO: 303 | 6891 |
| CII4725 | VL | PRT | WO2016023019; SEQ ID NO: 390 | 6892 |
| CII4726 | VL | PRT | WO2016023019; SEQ ID NO: 313 | 6893 |
| CII4727 | VL | PRT | WO2016023019; SEQ ID NO: 257 | 6894 |
| CII4728 | VL | PRT | WO2016023019; SEQ ID NO: 295 | 6895 |
| CII4729 | VL | PRT | WO2017062672; SEQ ID NO: 353 | 6896 |
| CII4730 | VL | PRT | WO2017062672; SEQ ID NO: 352 | 6897 |
| CII4731 | VL | PRT | WO2017062672; SEQ ID NO: 280 | 6898 |
| CII4732 | VL | PRT | WO2017062672; SEQ ID NO: 629 | 6899 |
| CII4733 | VL | PRT | WO2017062672; SEQ ID NO: 279 | 6900 |
| CII4734 | VL | PRT | WO2017062672; SEQ ID NO: 631 | 6901 |
| CII4735 | VL | PRT | WO2017062672; SEQ ID NO: 338 | 6902 |
| CII4736 | VL | PRT | WO2017062672; SEQ ID NO: 341 | 6903 |
| CII4737 | VL | PRT | WO2017062672; SEQ ID NO: 371 | 6904 |
| CII4738 | VL | PRT | WO2017062672; SEQ ID NO: 375 | 6905 |
| CII4739 | VL | PRT | WO2017062672; SEQ ID NO: 293 | 6906 |
| CII4740 | VL | PRT | WO2017062672; SEQ ID NO: 294 | 6907 |
| CII4741 | VL | PRT | WO2017062672; SEQ ID NO: 328 | 6908 |
| CII4742 | VL | PRT | WO2017062672; SEQ ID NO: 327 | 6909 |
| CII4743 | VL | PRT | WO2017062672; SEQ ID NO: 269 | 6910 |
| CII4744 | VL | PRT | WO2017062672; SEQ ID NO: 265 | 6911 |
| CII4745 | VL | PRT | WO2016023019; SEQ ID NO: 352 | 6912 |
| CII4746 | VL | PRT | WO2016023019; SEQ ID NO: 387 | 6913 |
| CII4747 | VL | PRT | WO2016023019; SEQ ID NO: 389 | 6914 |
| CII4748 | VL | PRT | WO2017062672; SEQ ID NO: 359 | 6915 |
| CII4749 | VL | PRT | WO2017062672; SEQ ID NO: 257 | 6916 |
| CII4750 | VL | PRT | WO2017062672; SEQ ID NO: 383 | 6917 |
| CII4751 | VL | PRT | WO2017062672; SEQ ID NO: 392 | 6918 |
| CII4752 | VL | PRT | WO2017062672; SEQ ID NO: 245 | 6919 |
| CII4753 | VL | PRT | WO2017062672; SEQ ID NO: 303 | 6920 |
| CII4754 | VL | PRT | WO2017062672; SEQ ID NO: 235 | 6921 |
| CII4755 | VL | PRT | WO2017062672; SEQ ID NO: 619 | 6922 |
| CII4756 | VL | PRT | WO2017062672; SEQ ID NO: 606 | 6923 |
| CII4757 | VL | PRT | WO2017062672; SEQ ID NO: 683 | 6924 |
| CII4758 | VL | PRT | WO2017062672; SEQ ID NO: 223 | 6925 |
| CII4759 | VL | PRT | WO2017062672; SEQ ID NO: 314 | 6926 |
| CII4760 | VL | PRT | WO2016023019; SEQ ID NO: 342 | 6927 |
| CII4761 | VL | PRT | WO2016023019; SEQ ID NO: 394 | 6928 |
| CII4762 | VL | PRT | WO2017062672; SEQ ID NO: 351 | 6929 |
| CII4763 | VL | PRT | WO2017062672; SEQ ID NO: 282 | 6930 |
| CII4764 | VL | PRT | WO2017062672; SEQ ID NO: 630 | 6931 |
| CII4765 | VL | PRT | WO2017062672; SEQ ID NO: 340 | 6932 |
| CII4766 | VL | PRT | WO2017062672; SEQ ID NO: 374 | 6933 |
| CII4767 | VL | PRT | WO2017062672; SEQ ID NO: 296 | 6934 |
| CII4768 | VL | PRT | WO2017062672; SEQ ID NO: 326 | 6935 |
| CII4769 | VL | PRT | WO2017062672; SEQ ID NO: 266 | 6936 |
| CII4770 | VL | PRT | WO2017062672; SEQ ID NO: 813 | 6937 |
| CII4771 | VL | PRT | WO2017062672; SEQ ID NO: 613 | 6938 |
| CII4772 | VL | PRT | WO2017062672; SEQ ID NO: 727 | 6939 |
| CII4773 | VL | PRT | WO2017062672; SEQ ID NO: 729 | 6940 |
| CII4774 | VL | PRT | WO2017062672; SEQ ID NO: 724 | 6941 |
| CII4775 | VL | PRT | WO2017062672; SEQ ID NO: 730 | 6942 |
| CII4776 | VL | PRT | WO2017062672; SEQ ID NO: 726 | 6943 |
| CII4777 | VL | PRT | WO2017062672; SEQ ID NO: 364 | 6944 |
| CII4778 | VL | PRT | WO2017062672; SEQ ID NO: 255 | 6945 |
| CII4779 | VL | PRT | WO2017062672; SEQ ID NO: 377 | 6946 |
| CII4780 | VL | PRT | WO2017062672; SEQ ID NO: 388 | 6947 |
| CII4781 | VL | PRT | WO2017062672; SEQ ID NO: 244 | 6948 |
| CII4782 | VL | PRT | WO2017062672; SEQ ID NO: 300 | 6949 |
| CII4783 | VL | PRT | WO2017062672; SEQ ID NO: 231 | 6950 |
| CII4784 | VL | PRT | WO2017062672; SEQ ID NO: 614 | 6951 |
| CII4785 | VL | PRT | WO2017062672; SEQ ID NO: 603 | 6952 |
| CII4786 | VL | PRT | WO2017062672; SEQ ID NO: 680 | 6953 |
| CII4787 | VL | PRT | WO2016023019; SEQ ID NO: 281 | 6954 |
| CII4788 | VL | PRT | WO2016023019; SEQ ID NO: 377 | 6955 |
| CII4789 | VL | PRT | WO2016023019; SEQ ID NO: 375 | 6956 |
| CII4790 | VL | PRT | WO2017062672; SEQ ID NO: 229 | 6957 |
| CII4791 | VL | PRT | WO2017062672; SEQ ID NO: 320 | 6958 |
| CII4792 | VL | PRT | WO2017062672; SEQ ID NO: 347 | 6959 |
| CII4793 | VL | PRT | WO2017062672; SEQ ID NO: 277 | 6960 |
| CII4794 | VL | PRT | WO2017062672; SEQ ID NO: 627 | 6961 |
| CII4795 | VL | PRT | WO2017062672; SEQ ID NO: 336 | 6962 |
| CII4796 | VL | PRT | WO2017062672; SEQ ID NO: 368 | 6963 |
| CII4797 | VL | PRT | WO2017062672; SEQ ID NO: 290 | 6964 |
| CII4798 | VL | PRT | WO2017062672; SEQ ID NO: 332 | 6965 |
| CII4799 | VL | PRT | WO2017062672; SEQ ID NO: 273 | 6966 |
| CII4800 | VL | PRT | WO2017062672; SEQ ID NO: 728 | 6967 |
| CII4801 | VL | PRT | WO2017062672; SEQ ID NO: 362 | 6968 |
| CII4802 | VL | PRT | WO2017062672; SEQ ID NO: 254 | 6969 |
| CII4803 | VL | PRT | WO2017062672; SEQ ID NO: 379 | 6970 |
| CII4804 | VL | PRT | WO2017062672; SEQ ID NO: 389 | 6971 |
| CII4805 | VL | PRT | WO2017062672; SEQ ID NO: 243 | 6972 |
| CII4806 | VL | PRT | WO2017062672; SEQ ID NO: 301 | 6973 |
| CII4807 | VL | PRT | WO2017062672; SEQ ID NO: 233 | 6974 |
| CII4808 | VL | PRT | WO2017062672; SEQ ID NO: 615 | 6975 |
| CII4809 | VL | PRT | WO2017062672; SEQ ID NO: 605 | 6976 |
| CII4810 | VL | PRT | WO2017062672; SEQ ID NO: 681 | 6977 |
| CII4811 | VL | PRT | WO2017062672; SEQ ID NO: 227 | 6978 |
| CII4812 | VL | PRT | WO2017062672; SEQ ID NO: 319 | 6979 |
| CII4813 | VL | PRT | WO2017062672; SEQ ID NO: 346 | 6980 |
| CII4814 | VL | PRT | WO2016023019; SEQ ID NO: 381 | 6981 |
| CII4815 | VL | PRT | WO2017062672; SEQ ID NO: 276 | 6982 |
| CII4816 | VL | PRT | WO2017062672; SEQ ID NO: 626 | 6983 |
| CII4817 | VL | PRT | WO2019028292; SEQ ID NO: 116 | 6984 |
| CII4818 | VL | PRT | WO2017062672; SEQ ID NO: 335 | 6985 |
| CII4819 | VL | PRT | WO2017062672; SEQ ID NO: 367 | 6986 |
| CII4820 | VL | PRT | WO2017062672; SEQ ID NO: 289 | 6987 |
| CII4821 | VL | PRT | WO2017062672; SEQ ID NO: 330 | 6988 |
| CII4822 | VL | PRT | WO2017062672; SEQ ID NO: 271 | 6989 |
| CII4823 | VL | PRT | WO2017062672; SEQ ID NO: 843 | 6990 |
| CII4824 | VL | PRT | WO2019028292; SEQ ID NO: 114 | 6991 |
| CII4825 | VL | PRT | WO2017062672; SEQ ID NO: 376 | 6992 |
| CII4826 | VL | PRT | WO2017062672; SEQ ID NO: 219 | 6993 |
| CII4827 | VL | PRT | WO2017062672; SEQ ID NO: 344 | 6994 |
| CII4828 | VL | PRT | WO2017062672; SEQ ID NO: 725 | 6995 |
| CII4829 | VL | PRT | WO2019028292; SEQ ID NO: 117 | 6996 |
| CII4830 | VL | PRT | WO2019028292; SEQ ID NO: 104 | 6997 |
| CII4831 | VL | PRT | WO2019028292; SEQ ID NO: 111 | 6998 |
| CII4832 | VL | PRT | WO2019028292; SEQ ID NO: 112 | 6999 |
| CII4833 | VL | PRT | WO2017062672; SEQ ID NO: 363 | 7000 |
| CII4834 | VL | PRT | WO2017062672; SEQ ID NO: 253 | 7001 |
| CII4835 | VL | PRT | WO2017062672; SEQ ID NO: 378 | 7002 |
| CII4836 | VL | PRT | WO2017062672; SEQ ID NO: 390 | 7003 |
| CII4837 | VL | PRT | WO2017062672; SEQ ID NO: 242 | 7004 |
| CII4838 | VL | PRT | WO2017062672; SEQ ID NO: 302 | 7005 |
| CII4839 | VL | PRT | WO2017062672; SEQ ID NO: 232 | 7006 |
| CII4840 | VL | PRT | WO2017062672; SEQ ID NO: 616 | 7007 |
| CII4841 | VL | PRT | WO2017062672; SEQ ID NO: 604 | 7008 |
| CII4842 | VL | PRT | WO2017062672; SEQ ID NO: 682 | 7009 |
| CII4843 | VL | PRT | WO2017062672; SEQ ID NO: 228 | 7010 |
| CII4844 | VL | PRT | WO2017062672; SEQ ID NO: 321 | 7011 |
| CII4845 | VL | PRT | WO2017062672; SEQ ID NO: 345 | 7012 |
| CII4846 | VL | PRT | WO2017062672; SEQ ID NO: 275 | 7013 |
| CII4847 | VL | PRT | WO2017062672; SEQ ID NO: 625 | 7014 |
| CII4848 | VL | PRT | WO2017062672; SEQ ID NO: 334 | 7015 |
| CII4849 | VL | PRT | WO2017062672; SEQ ID NO: 366 | 7016 |
| CII4850 | VL | PRT | WO2017062672; SEQ ID NO: 288 | 7017 |
| CII4851 | VL | PRT | WO2017062672; SEQ ID NO: 331 | 7018 |
| CII4852 | VL | PRT | WO2017062672; SEQ ID NO: 272 | 7019 |
| CII4853 | VL | PRT | WO2017062672; SEQ ID NO: 812 | 7020 |
| CII4854 | VL | PRT | WO2017062672; SEQ ID NO: 811 | 7021 |
| CII4855 | VL | PRT | WO2017062672; SEQ ID NO: 816 | 7022 |
| CII4856 | VL | PRT | WO2017062672; SEQ ID NO: 624 | 7023 |
| CII4857 | VL | PRT | WO2017062672; SEQ ID NO: 850 | 7024 |
| CII4858 | VL | PRT | WO2017062672; SEQ ID NO: 365 | 7025 |
| CII4859 | VL | PRT | WO2017062672; SEQ ID NO: 287 | 7026 |
| CII4860 | VL | PRT | WO2019028292; SEQ ID NO: 115 | 7027 |
| CII4861 | VL | PRT | WO2017062672; SEQ ID NO: 844 | 7028 |
| CII4862 | VL | PRT | WO2019028292; SEQ ID NO: 92 | 7029 |
| CII4863 | VL | PRT | WO2019028292; SEQ ID NO: 93 | 7030 |
| CII4864 | VL | PRT | WO2019028292; SEQ ID NO: 94 | 7031 |
| CII4865 | VL | PRT | WO2019028292; SEQ ID NO: 98 | 7032 |
| CII4886 | VL | PRT | WO2019028292; SEQ ID NO: 108 | 7033 |
| CII4867 | VL | PRT | WO2019028292; SEQ ID NO: 109 | 7034 |
| CII4868 | VL | PRT | WO2017062672; SEQ ID NO: 849 | 7035 |
| CII4889 | VL | PRT | WO2017062672; SEQ ID NO: 298 | 7036 |
| CII4870 | VL | PRT | WO2017062672; SEQ ID NO: 333 | 7037 |
| CII4871 | VL | PRT | WO2017062672; SEQ ID NO: 274 | 7038 |
| CII4872 | VL | PRT | WO2017062672; SEQ ID NO: 310 | 7039 |
| CII4873 | VL | PRT | WO2017062672; SEQ ID NO: 252 | 7040 |
| CII4874 | VL | PRT | WO2017062672; SEQ ID NO: 241 | 7041 |
| CII4875 | VL | PRT | WO2017062672; SEQ ID NO: 821 | 7042 |
| CII4876 | VL | PRT | WO2017062672; SEQ ID NO: 260 | 7043 |
| CII4877 | VL | PRT | WO2017062672; SEQ ID NO: 381 | 7044 |
| CII4878 | VL | PRT | WO2017062672; SEQ ID NO: 395 | 7045 |
| CII4879 | VL | PRT | WO2017062672; SEQ ID NO: 250 | 7046 |
| CII4880 | VL | PRT | WO2017062672; SEQ ID NO: 307 | 7047 |
| CII4881 | VL | PRT | WO2017062672; SEQ ID NO: 239 | 7048 |
| CII4882 | VL | PRT | WO2017062672; SEQ ID NO: 622 | 7049 |
| CII4883 | VL | PRT | WO2017062672; SEQ ID NO: 609 | 7050 |
| CII4884 | VL | PRT | WO2017062672; SEQ ID NO: 687 | 7051 |
| CII4885 | VL | PRT | WO2017062672; SEQ ID NO: 222 | 7052 |
| CII4886 | VL | PRT | WO2017062672; SEQ ID NO: 313 | 7053 |
| CII4887 | VL | PRT | WO2016023019; SEQ ID NO: 396 | 7054 |
| CII4888 | VL | PRT | WO2016023019; SEQ ID NO: 346 | 7055 |
| CII4889 | VL | PRT | WO2016023019; SEQ ID NO: 255 | 7056 |
| CII4890 | VL | PRT | WO2016023019; SEQ ID NO: 329 | 7057 |
| CII4891 | VL | PRT | WO2016023019; SEQ ID NO: 253 | 7058 |
| CII4892 | VL | PRT | WO2016023019; SEQ ID NO: 291 | 7059 |
| CII4893 | VL | PRT | WO2016023019; SEQ ID NO: 350 | 7060 |
| CII4894 | VL | PRT | WO2016023019; SEQ ID NO: 340 | 7061 |
| CII4895 | VL | PRT | WO2016023019; SEQ ID NO: 379 | 7062 |
| CII4896 | VL | PRT | WO2016023019; SEQ ID NO: 301 | 7063 |
| CII4897 | VL | PRT | WO2016023019; SEQ ID NO: 356 | 7064 |
| CII4898 | VL | PRT | WO2016023019; SEQ ID NO: 287 | 7065 |
| CII4899 | VL | PRT | WO2016023019; SEQ ID NO: 259 | 7066 |
| CII4900 | VL | PRT | WO2016023019; SEQ ID NO: 311 | 7067 |
| CII4901 | VL | PRT | WO2016023019; SEQ ID NO: 307 | 7068 |
| CII4902 | VL | PRT | WO2016023019; SEQ ID NO: 336 | 7069 |
| CII4903 | VL | PRT | WO2016023019; SEQ ID NO: 328 | 7070 |
| CII4904 | VL | PRT | WO2016023019; SEQ ID NO: 363 | 7071 |
| CII4905 | VL | PRT | WO2016023019; SEQ ID NO: 309 | 7072 |
| CII4906 | VL | PRT | WO2016023019; SEQ ID NO: 361 | 7073 |
| CII4907 | VL | PRT | WO2016023019; SEQ ID NO: 359 | 7074 |
| CII4908 | VL | PRT | WO2016023019; SEQ ID NO: 371 | 7075 |
| CII4909 | VL | PRT | WO2016023019; SEQ ID NO: 364 | 7076 |
| CII4910 | VL | PRT | WO2016023019; SEQ ID NO: 385 | 7077 |
| CII4911 | VL | PRT | WO2016023019; SEQ ID NO: 373 | 7078 |
| CII4912 | VL | PRT | WO2016023019; SEQ ID NO: 267 | 7079 |
| CII4913 | VL | PRT | WO2016023019; SEQ ID NO: 289 | 7080 |
| CII4914 | VL | PRT | WO2016023019; SEQ ID NO: 299 | 7081 |
| CII4915 | VL | PRT | WO2016023019; SEQ ID NO: 383 | 7082 |
| CII4916 | VL | PRT | WO2016023019; SEQ ID NO: 316 | 7083 |
| CII4917 | VL | PRT | WO2016023019; SEQ ID NO: 348 | 7084 |
| CII4918 | VL | PRT | WO2016023019; SEQ ID NO: 325 | 7085 |
| CII4919 | VL | PRT | WO2017062672; SEQ ID NO: 348 | 7086 |
| CII4920 | VL | PRT | WO2017062672; SEQ ID NO: 278 | 7087 |
| CII4921 | VL | PRT | WO2017062672; SEQ ID NO: 628 | 7088 |
| CII4922 | VL | PRT | WO2017062672; SEQ ID NO: 337 | 7089 |
| CII4923 | VL | PRT | WO2017062672; SEQ ID NO: 369 | 7090 |
| CII4924 | VL | PRT | WO2017062672; SEQ ID NO: 291 | 7091 |
| CII4925 | VL | PRT | WO2017062672; SEQ ID NO: 324 | 7092 |
| CII4926 | VL | PRT | WO2017062672; SEQ ID NO: 264 | 7093 |
| CII4927 | VL | PRT | WO2016023019; SEQ ID NO: 245 | 7094 |
| CII4928 | VL | PRT | WO2017062672; SEQ ID NO: 361 | 7095 |
| CII4929 | VL | PRT | WO2017062672; SEQ ID NO: 262 | 7096 |
| CII4930 | VL | PRT | WO2017062672; SEQ ID NO: 384 | 7097 |
| CII4931 | VL | PRT | WO2017062672; SEQ ID NO: 397 | 7098 |
| CII4932 | VL | PRT | WO2017062672; SEQ ID NO: 251 | 7099 |
| CII4933 | VL | PRT | WO2017062672; SEQ ID NO: 309 | 7100 |
| CII4934 | VL | PRT | WO2017062672; SEQ ID NO: 240 | 7101 |
| CII4935 | VL | PRT | WO2017062672; SEQ ID NO: 623 | 7102 |
| CII4936 | VL | PRT | WO2017062672; SEQ ID NO: 612 | 7103 |
| CII4937 | VL | PRT | WO2017062672; SEQ ID NO: 689 | 7104 |
| CII4938 | VL | PRT | WO2017062672; SEQ ID NO: 226 | 7105 |
| CII4939 | VL | PRT | WO2017062672; SEQ ID NO: 318 | 7106 |
| CII4940 | VL | PRT | WO2016023019; SEQ ID NO: 263 | 7107 |
| CII4941 | VL | PRT | WO2016023019; SEQ ID NO: 278 | 7108 |
| CII4942 | VL | PRT | WO2016023019; SEQ ID NO: 285 | 7109 |
| CII4943 | VL | PRT | WO2016023019; SEQ ID NO: 261 | 7110 |
| CII4944 | VL | PRT | WO2016023019; SEQ ID NO: 293 | 7111 |
| CII4945 | VL | PRT | WO2016023019; SEQ ID NO: 330 | 7112 |
| CII4946 | VL | PRT | WO2016023019; SEQ ID NO: 318 | 7113 |
| CII4947 | VL | PRT | WO2016023019; SEQ ID NO: 333 | 7114 |
| CII4948 | VL | PRT | WO2016023019; SEQ ID NO: 327 | 7115 |
| CII4949 | VL | PRT | WO2016023019; SEQ ID NO: 358 | 7116 |
| CII4950 | VL | PRT | WO2017062672; SEQ ID NO: 354 | 7117 |
| CII4951 | VL | PRT | WO2017062672; SEQ ID NO: 284 | 7118 |
| CII4952 | VL | PRT | WO2017062672; SEQ ID NO: 634 | 7119 |
| CII4953 | VL | PRT | WO2017062672; SEQ ID NO: 343 | 7120 |
| CII4954 | VL | PRT | WO2017062672; SEQ ID NO: 372 | 7121 |
| CII4955 | VL | PRT | WO2017062672; SEQ ID NO: 297 | 7122 |
| CII4956 | VL | PRT | WO2017062672; SEQ ID NO: 329 | 7123 |
| CII4957 | VL | PRT | WO2017062672; SEQ ID NO: 270 | 7124 |
| CII4958 | VL | PRT | WO2016023019; SEQ ID NO: 338 | 7125 |
| CII4959 | VL | PRT | WO2016023019; SEQ ID NO: 321 | 7126 |
| CII4960 | VL | PRT | WO2017062672; SEQ ID NO: 360 | 7127 |
| CII4961 | VL | PRT | WO2017062672; SEQ ID NO: 261 | 7128 |
| CII4962 | VL | PRT | WO2017062672; SEQ ID NO: 382 | 7129 |
| CII4963 | VL | PRT | WO2017062672; SEQ ID NO: 393 | 7130 |
| CII4964 | VL | PRT | WO2017062672; SEQ ID NO: 249 | 7131 |
| CII4965 | VL | PRT | WO2017062672; SEQ ID NO: 305 | 7132 |
| CII4966 | VL | PRT | WO2017062672; SEQ ID NO: 236 | 7133 |
| CII4967 | VL | PRT | WO2017062672; SEQ ID NO: 618 | 7134 |
| CII4968 | VL | PRT | WO2017062672; SEQ ID NO: 607 | 7135 |
| CII4969 | VL | PRT | WO2017062672; SEQ ID NO: 685 | 7136 |
| CII4970 | VL | PRT | WO2017062672; SEQ ID NO: 220 | 7137 |
| CII4971 | VL | PRT | WO2017062672; SEQ ID NO: 315 | 7138 |
| CII4972 | VL | PRT | WO2016023019; SEQ ID NO: 354 | 7139 |
| CII4973 | VL | PRT | WO2016023019; SEQ ID NO: 370 | 7140 |
| CII4974 | VL | PRT | WO2016023019; SEQ ID NO: 323 | 7141 |
| CII4975 | VL | PRT | WO2016023019; SEQ ID NO: 243 | 7142 |
| CII4976 | VL | PRT | WO2016023019; SEQ ID NO: 413 | 7143 |
| CII4977 | VL | PRT | WO2016023019; SEQ ID NO: 249 | 7144 |
| CII4978 | VL | PRT | WO2017062672; SEQ ID NO: 349 | 7145 |
| CII4979 | VL | PRT | WO2017062672; SEQ ID NO: 281 | 7146 |
| CII4980 | VL | PRT | WO2017062672; SEQ ID NO: 633 | 7147 |
| CII4981 | VL | PRT | WO2017062672; SEQ ID NO: 339 | 7148 |
| CII4982 | VL | PRT | WO2017062672; SEQ ID NO: 370 | 7149 |
| CII4983 | VL | PRT | WO2017062672; SEQ ID NO: 295 | 7150 |
| CII4984 | VL | PRT | WO2017062672; SEQ ID NO: 323 | 7151 |
| CII4985 | VL | PRT | WO2017062672; SEQ ID NO: 267 | 7152 |
| CII4986 | VL | PRT | WO2016023019; SEQ ID NO: 366 | 7153 |
| CII4987 | VL | PRT | WO2016023019; SEQ ID NO: 411 | 7154 |
| CII4988 | VL | PRT | WO2016023019; SEQ ID NO: 279 | 7155 |
| CII4989 | VL | PRT | WO2016023019; SEQ ID NO: 251 | 7156 |
| CII4990 | VL | PRT | WO2017062672; SEQ ID NO: 355 | 7157 |
| CII4991 | VL | PRT | WO2019028292; SEQ ID NO: 103 | 7158 |
| CII4992 | VL | PRT | WO2019028292; SEQ ID NO: 95 | 7159 |
| CII4993 | VL | PRT | WO2019028292; SEQ ID NO: 97 | 7160 |
| CII4994 | VL | PRT | WO2019028292; SEQ ID NO: 110 | 7161 |
| CII4995 | VL | PRT | WO2019028292; SEQ ID NO: 113 | 7162 |
| CII4996 | VL | PRT | WO2019028292; SEQ ID NO: 99 | 7163 |
| CII4997 | VL | PRT | WO2019028292; SEQ ID NO: 101 | 7164 |
| CII4998 | VL | PRT | WO2019028292; SEQ ID NO: 102 | 7165 |
| CII4999 | VL | PRT | WO2019028292; SEQ ID NO: 106 | 7166 |
| CII5000 | VL | PRT | WO2019028292; SEQ ID NO: 105 | 7167 |
| CII5001 | VL | PRT | WO2019028292; SEQ ID NO: 100 | 7168 |
| CII5002 | VL | PRT | WO2019028292; SEQ ID NO: 96 | 7169 |
| CII5003 | VL | PRT | WO2019028292; SEQ ID NO: 118 | 7170 |
| CII5004 | VL | PRT | WO2019028292; SEQ ID NO: 107 | 7171 |
| CII5005 | VL | PRT | WO2017062672; SEQ ID NO: 815 | 7172 |
| CII5006 | VL | PRT | WO2017062672; SEQ ID NO: 299 | 7173 |
| CII5007 | VL | PRT | WO2017062672; SEQ ID NO: 810 | 7174 |
| CII5008 | VL | PRT | WO2017062672; SEQ ID NO: 286 | 7175 |
| CII5009 | VL | PRT | WO2017062672; SEQ ID NO: 311 | 7176 |
| CII5010 | VL | PRT | WO2017062672; SEQ ID NO: 679 | 7177 |
| CII5011 | VL | PRT | WO2017062672; SEQ ID NO: 602 | 7178 |
| CII5012 | VL | PRT | WO2017062672; SEQ ID NO: 398 | 7179 |
| CII5013 | VL | PRT | WO2017062672; SEQ ID NO: 230 | 7180 |
| CII5014 | VL | PRT | WO2017062672; SEQ ID NO: 387 | 7181 |
| CII5015 | VL | PRT | WO2017062672; SEQ ID NO: 285 | 7182 |
| CII5016 | VL | PRT | WO2017062672; SEQ ID NO: 263 | 7183 |
| CII5017 | VL | PRT | WO2017062672; SEQ ID NO: 809 | 7184 |
| CII5018 | VL | PRT | WO2014028776; SEQ ID NO: 74 | 7185 |
| CII5019 | VL | PRT | US20180221480; SEQ ID NO: 12 | 7186 |
| CII5020 | VL | PRT | US20180221480; SEQ ID NO: 7 | 7187 |
| CII5021 | VL | PRT | US20180221480; SEQ ID NO: 13 | 7188 |
| CII5022 | VL | PRT | WO2018140510; SEQ ID NO: 2 | 7189 |
| CII5023 | VL | PRT | US20180051086; SEQ ID NO: 5 | 7190 |
| CII5024 | VL | PRT | US20180051086; SEQ ID NO: 4 | 7191 |
| CII5025 | VR | DNA | US20180201692; SEQ ID NO: 10 | 7192 |
| CII5026 | VR | DNA | US20180201692; SEQ ID NO: 13 | 7193 |
| CII5027 | VR | DNA | US20180201692; SEQ ID NO: 15 | 7194 |
| CII5028 | VR | DNA | US20180201692; SEQ ID NO: 7 | 7195 |
| CII5029 | VR | DNA | US20180201692; SEQ ID NO: 5 | 7196 |
| CII5030 | VR | DNA | US20180201692; SEQ ID NO: 8 | 7197 |
| CII5031 | VR | DNA | US20180201692; SEQ ID NO: 12 | 7198 |
| CII5032 | VR | DNA | US20180201692; SEQ ID NO: 6 | 7199 |
| CII5033 | VR | DNA | US20180201692; SEQ ID NO: 4 | 7200 |
| CII5034 | VR | DNA | US20180201692; SEQ ID NO: 3 | 7201 |
| CII5035 | VR | DNA | US20180201692; SEQ ID NO: 14 | 7202 |
| CII5036 | VR | DNA | US20180201692; SEQ ID NO: 11 | 7203 |
| CII5037 | VR | DNA | US20180201692; SEQ ID NO: 16 | 7204 |
| CII5038 | VR | DNA | US20180201692; SEQ ID NO: 9 | 7205 |
| CII5039 | VR | PRT | US20190085084; SEQ ID NO: 441 | 7206 |
| CII5040 | VR | PRT | US20190085084; SEQ ID NO: 441 | 7207 |
| CII5041 | VR | PRT | WO2016164637; SEQ ID NO: 441 | 7208 |
| CII5042 | VR | PRT | US20190085084; SEQ ID NO: 438 | 7209 |
| CII5043 | VR | PRT | US20190085084; SEQ ID NO: 438 | 7210 |
| CII5044 | VR | PRT | WO2016164637; SEQ ID NO: 438 | 7211 |
| CII5045 | VR | PRT | US20190085084; SEQ ID NO: 460 | 7212 |
| CII5046 | VR | PRT | US20190085084; SEQ ID NO: 460 | 7213 |
| CII5047 | VR | PRT | WO2016164637; SEQ ID NO: 460 | 7214 |
| CII5048 | VR | PRT | US20190085084; SEQ ID NO: 354 | 7215 |
| CII5049 | VR | PRT | WO2016164637; SEQ ID NO: 354 | 7216 |
| CII5050 | VR | PRT | WO2016164637; SEQ ID NO: 319 | 7217 |
| CII5051 | VR | PRT | US20190085084; SEQ ID NO: 341 | 7218 |
| CII5052 | VR | PRT | WO2016164637; SEQ ID NO: 341 | 7219 |
| CII5053 | VR | PRT | US20190085084; SEQ ID NO: 350 | 7220 |
| CII5054 | VR | PRT | WO2016164637; SEQ ID NO: 350 | 7221 |
| CII5055 | VR | PRT | US20190085084; SEQ ID NO: 364 | 7222 |
| CII5056 | VR | PRT | US20190085084; SEQ ID NO: 364 | 7223 |
| CII5057 | VR | PRT | WO2016164637; SEQ ID NO: 364 | 7224 |
| CII5058 | VR | PRT | US20190085084; SEQ ID NO: 421 | 7225 |
| CII5059 | VR | PRT | US20190085084; SEQ ID NO: 421 | 7226 |
| CII5060 | VR | PRT | WO2016164637; SEQ ID NO: 421 | 7227 |
| CII5061 | VR | PRT | US20190085084; SEQ ID NO: 453 | 7228 |
| CII5062 | VR | PRT | US20190085084; SEQ ID NO: 453 | 7229 |
| CII5063 | VR | PRT | WO2016164637; SEQ ID NO: 453 | 7230 |
| CII5064 | VR | PRT | US20190085084; SEQ ID NO: 344 | 7231 |
| CII5065 | VR | PRT | US20190085084; SEQ ID NO: 344 | 7232 |
| CII5066 | VR | PRT | WO2016164637; SEQ ID NO: 344 | 7233 |
| CII5067 | VR | PRT | WO2016164637; SEQ ID NO: 356 | 7234 |
| CII5068 | VR | PRT | US20190085084; SEQ ID NO: 321 | 7235 |
| CII5069 | VR | PRT | US20190085084; SEQ ID NO: 321 | 7236 |
| CII5070 | VR | PRT | WO2016164637; SEQ ID NO: 321 | 7237 |
| CII5071 | VR | PRT | US20190085084; SEQ ID NO: 464 | 7238 |
| CII5072 | VR | PRT | US20190085084; SEQ ID NO: 464 | 7239 |
| CII5073 | VR | PRT | WO2016164637; SEQ ID NO: 464 | 7240 |
| CII5074 | VR | PRT | US20190085084; SEQ ID NO: 389 | 7241 |
| CII5075 | VR | PRT | US20190085084; SEQ ID NO: 389 | 7242 |
| CII5076 | VR | PRT | WO2016164637; SEQ ID NO: 389 | 7243 |
| CII5077 | VR | PRT | US20190085084; SEQ ID NO: 375 | 7244 |
| CII5078 | VR | PRT | US20190085084; SEQ ID NO: 375 | 7245 |
| CII5079 | VR | PRT | WO2016164637; SEQ ID NO: 375 | 7246 |
| CII5080 | VR | PRT | US20190085084; SEQ ID NO: 476 | 7247 |
| CII5081 | VR | PRT | US20190085084; SEQ ID NO: 476 | 7248 |
| CII5082 | VR | PRT | WO2016164637; SEQ ID NO: 476 | 7249 |
| CII5083 | VR | PRT | US20190085084; SEQ ID NO: 443 | 7250 |
| CII5084 | VR | PRT | US20190085084; SEQ ID NO: 443 | 7251 |
| CII5085 | VR | PRT | WO2016164637; SEQ ID NO: 443 | 7252 |
| CII5086 | VR | PRT | US20190085084; SEQ ID NO: 411 | 7253 |
| CII5087 | VR | PRT | US20190085084; SEQ ID NO: 411 | 7254 |
| CII5088 | VR | PRT | WO2016164637; SEQ ID NO: 411 | 7255 |
| CII5089 | VR | PRT | US20190085084; SEQ ID NO: 371 | 7256 |
| CII5090 | VR | PRT | WO2016164637; SEQ ID NO: 371 | 7257 |
| CII5091 | VR | PRT | US20190085084; SEQ ID NO: 439 | 7258 |
| CII5092 | VR | PRT | US20190085084; SEQ ID NO: 439 | 7259 |
| CII5093 | VR | PRT | WO2016164637; SEQ ID NO: 439 | 7260 |
| CII5094 | VR | PRT | WO2016164637; SEQ ID NO: 430 | 7261 |
| CII5095 | VR | PRT | US20190085084; SEQ ID NO: 370 | 7262 |
| CII5096 | VR | PRT | WO2016164637; SEQ ID NO: 370 | 7263 |
| CII5097 | VR | PRT | US20190085084; SEQ ID NO: 368 | 7264 |
| CII5098 | VR | PRT | US20190085084; SEQ ID NO: 368 | 7265 |
| CII5099 | VR | PRT | WO2016164637; SEQ ID NO: 368 | 7266 |
| CII5100 | VR | PRT | US20190085084; SEQ ID NO: 324 | 7267 |
| CII5101 | VR | PRT | WO2016164637; SEQ ID NO: 324 | 7268 |
| CII5102 | VR | PRT | US20190085084; SEQ ID NO: 470 | 7269 |
| CII5103 | VR | PRT | US20190085084; SEQ ID NO: 470 | 7270 |
| CII5104 | VR | PRT | WO2016164637; SEQ ID NO: 470 | 7271 |
| CII5105 | VR | PRT | US20190085084; SEQ ID NO: 681 | 7272 |
| CII5106 | VR | PRT | WO2016164637; SEQ ID NO: 681 | 7273 |
| CII5107 | VR | PRT | US20190085084; SEQ ID NO: 683 | 7274 |
| CII5108 | VR | PRT | WO2016164637; SEQ ID NO: 683 | 7275 |
| CII5109 | VR | PRT | US20190085084; SEQ ID NO: 682 | 7276 |
| CII5110 | VR | PRT | WO2016164637; SEQ ID NO: 682 | 7277 |
| CII5111 | VR | PRT | US20190085084; SEQ ID NO: 684 | 7278 |
| CII5112 | VR | PRT | WO2016164637; SEQ ID NO: 684 | 7279 |
| CII5113 | VR | PRT | US20190085084; SEQ ID NO: 413 | 7280 |
| CII5114 | VR | PRT | WO2016164637; SEQ ID NO: 413 | 7281 |
| CII5115 | VR | PRT | US20190085084; SEQ ID NO: 455 | 7282 |
| CII5116 | VR | PRT | US20190085084; SEQ ID NO: 455 | 7283 |
| CII5117 | VR | PRT | WO2016164637; SEQ ID NO: 455 | 7284 |
| CII5118 | VR | PRT | US20190085084; SEQ ID NO: 348 | 7285 |
| CII5119 | VR | PRT | WO2016164637; SEQ ID NO: 348 | 7286 |
| CII5120 | VR | PRT | US20190085084; SEQ ID NO: 427 | 7287 |
| CII5121 | VR | PRT | US20190085084; SEQ ID NO: 427 | 7288 |
| CII5122 | VR | PRT | WO2016164637; SEQ ID NO: 427 | 7289 |
| CII5123 | VR | PRT | US20190085084; SEQ ID NO: 474 | 7290 |
| CII5124 | VR | PRT | US20190085084; SEQ ID NO: 474 | 7291 |
| CII5125 | VR | PRT | WO2016164637; SEQ ID NO: 474 | 7292 |
| CII5126 | VR | PRT | US20190085084; SEQ ID NO: 337 | 7293 |
| CII5127 | VR | PRT | US20190085084; SEQ ID NO: 337 | 7294 |
| CII5128 | VR | PRT | WO2016164637; SEQ ID NO: 337 | 7295 |
| CII5129 | VR | PRT | US20190085084; SEQ ID NO: 328 | 7296 |
| CII5130 | VR | PRT | US20190085084; SEQ ID NO: 328 | 7297 |
| CII5131 | VR | PRT | WO2016164637; SEQ ID NO: 328 | 7298 |
| CII5132 | VR | PRT | US20190085084; SEQ ID NO: 409 | 7299 |
| CII5133 | VR | PRT | US20190085084; SEQ ID NO: 409 | 7300 |
| CII5134 | VR | PRT | WO2016164637; SEQ ID NO: 409 | 7301 |
| CII5135 | VR | PRT | US20190085084; SEQ ID NO: 399 | 7302 |
| CII5136 | VR | PRT | US20190085084; SEQ ID NO: 399 | 7303 |
| CII5137 | VR | PRT | WO2016164637; SEQ ID NO: 399 | 7304 |
| CII5138 | VR | PRT | US20190085084; SEQ ID NO: 317 | 7305 |
| CII5139 | VR | PRT | WO2016164637; SEQ ID NO: 317 | 7306 |
| CII5140 | VR | PRT | US20190085084; SEQ ID NO: 478 | 7307 |
| CII5141 | VR | PRT | US20190085084; SEQ ID NO: 478 | 7308 |
| CII5142 | VR | PRT | WO2016164637; SEQ ID NO: 478 | 7309 |
| CII5143 | VR | PRT | US20190085084; SEQ ID NO: 330 | 7310 |
| CII5144 | VR | PRT | US20190085084; SEQ ID NO: 330 | 7311 |
| CII5145 | VR | PRT | WO2016164637; SEQ ID NO: 330 | 7312 |
| CII5146 | VR | PRT | US20190085084; SEQ ID NO: 436 | 7313 |
| CII5147 | VR | PRT | US20190085084; SEQ ID NO: 436 | 7314 |
| CII5148 | VR | PRT | WO2016164637; SEQ ID NO: 436 | 7315 |
| CII5149 | VR | PRT | US20190085084; SEQ ID NO: 379 | 7316 |
| CII5150 | VR | PRT | US20190085084; SEQ ID NO: 379 | 7317 |
| CII5151 | VR | PRT | WO2016164637; SEQ ID NO: 379 | 7318 |
| CII5152 | VR | PRT | US20190085084; SEQ ID NO: 425 | 7319 |
| CII5153 | VR | PRT | WO2016164637; SEQ ID NO: 425 | 7320 |
| CII5154 | VR | PRT | US20190085084; SEQ ID NO: 451 | 7321 |
| CII5155 | VR | PRT | US20190085084; SEQ ID NO: 451 | 7322 |
| CII5156 | VR | PRT | WO2016164637; SEQ ID NO: 451 | 7323 |
| CII5157 | VR | PRT | US20190085084; SEQ ID NO: 395 | 7324 |
| CII5158 | VR | PRT | US20190085084; SEQ ID NO: 395 | 7325 |
| CII5159 | VR | PRT | WO2016164637; SEQ ID NO: 395 | 7326 |
| CII5160 | VR | PRT | US20190085084; SEQ ID NO: 429 | 7327 |
| CII5161 | VR | PRT | US20190085084; SEQ ID NO: 429 | 7328 |
| CII5162 | VR | PRT | WO2016164637; SEQ ID NO: 429 | 7329 |
| CII5163 | VR | PRT | US20190085084; SEQ ID NO: 397 | 7330 |
| CII5164 | VR | PRT | US20190085084; SEQ ID NO: 397 | 7331 |
| CII5165 | VR | PRT | WO2016164637; SEQ ID NO: 397 | 7332 |
| CII5166 | VR | PRT | US20190085084; SEQ ID NO: 401 | 7333 |
| CII5167 | VR | PRT | US20190085084; SEQ ID NO: 401 | 7334 |
| CII5168 | VR | PRT | WO2016164637; SEQ ID NO: 401 | 7335 |
| CII5169 | VR | PRT | WO2016164637; SEQ ID NO: 472 | 7336 |
| CII5170 | VR | PRT | US20190085084; SEQ ID NO: 360 | 7337 |
| CII5171 | VR | PRT | US20190085084; SEQ ID NO: 360 | 7338 |
| CII5172 | VR | PRT | WO2016164637; SEQ ID NO: 360 | 7339 |
| CII5173 | VR | PRT | US20190085084; SEQ ID NO: 362 | 7340 |
| CII5174 | VR | PRT | US20190085084; SEQ ID NO: 362 | 7341 |
| CII5175 | VR | PRT | WO2016164637; SEQ ID NO: 362 | 7342 |
| CII5176 | VR | PRT | US20190085084; SEQ ID NO: 391 | 7343 |
| CII5177 | VR | PRT | US20190085084; SEQ ID NO: 391 | 7344 |
| CII5178 | VR | PRT | WO2016164637; SEQ ID NO: 391 | 7345 |
| CII5179 | VR | PRT | US20190085084; SEQ ID NO: 457 | 7346 |
| CII5180 | VR | PRT | US20190085084; SEQ ID NO: 457 | 7347 |
| CII5181 | VR | PRT | WO2016164637; SEQ ID NO: 457 | 7348 |
| CII5182 | VR | PRT | US20190085084; SEQ ID NO: 667 | 7349 |
| CII5183 | VR | PRT | WO2016164637; SEQ ID NO: 667 | 7350 |
| CII5184 | VR | PRT | US20190085084; SEQ ID NO: 432 | 7351 |
| CII5185 | VR | PRT | US20190085084; SEQ ID NO: 432 | 7352 |
| CII5186 | VR | PRT | WO2016164637; SEQ ID NO: 432 | 7353 |
| CII5187 | VR | PRT | WO2016164637; SEQ ID NO: 343 | 7354 |
| CII5188 | VR | PRT | US20190085084; SEQ ID NO: 323 | 7355 |
| CII5189 | VR | PRT | US20190085084; SEQ ID NO: 323 | 7356 |
| CII5190 | VR | PRT | WO2016164637; SEQ ID NO: 323 | 7357 |
| CII5191 | VR | PRT | US20190085084; SEQ ID NO: 326 | 7358 |
| CII5192 | VR | PRT | US20190085084; SEQ ID NO: 326 | 7359 |
| CII5193 | VR | PRT | WO2016164637; SEQ ID NO: 326 | 7360 |
| CII5194 | VR | PRT | US20190085084; SEQ ID NO: 334 | 7361 |
| CII5195 | VR | PRT | US20190085084; SEQ ID NO: 334 | 7362 |
| CII5196 | VR | PRT | WO2016164637; SEQ ID NO: 334 | 7363 |
| CII5197 | VR | PRT | US20190085084; SEQ ID NO: 332 | 7364 |
| CII5198 | VR | PRT | US20190085084; SEQ ID NO: 332 | 7365 |
| CII5199 | VR | PRT | WO2016164637; SEQ ID NO: 332 | 7366 |
| CII5200 | VR | PRT | US20190085084; SEQ ID NO: 458 | 7367 |
| CII5201 | VR | PRT | US20190085084; SEQ ID NO: 458 | 7368 |
| CII5202 | VR | PRT | WO2016164637; SEQ ID NO: 458 | 7369 |
| CII5203 | VR | PRT | US20190085084; SEQ ID NO: 407 | 7370 |
| CII5204 | VR | PRT | US20190085084; SEQ ID NO: 407 | 7371 |
| CII5205 | VR | PRT | WO2016164637; SEQ ID NO: 407 | 7372 |
| CII5206 | VR | PRT | US20190085084; SEQ ID NO: 447 | 7373 |
| CII5207 | VR | PRT | US20190085084; SEQ ID NO: 447 | 7374 |
| CII5208 | VR | PRT | WO2016164637; SEQ ID NO: 447 | 7375 |
| CII5209 | VR | PRT | US20190085084; SEQ ID NO: 352 | 7376 |
| CII5210 | VR | PRT | WO2016164637; SEQ ID NO: 352 | 7377 |
| CII5211 | VR | PRT | US20190085084; SEQ ID NO: 396 | 7378 |
| CII5212 | VR | PRT | US20190085084; SEQ ID NO: 396 | 7379 |
| CII5213 | VR | PRT | WO2016164637; SEQ ID NO: 396 | 7380 |
| CII5214 | VR | PRT | US20190085084; SEQ ID NO: 390 | 7381 |
| CII5215 | VR | PRT | US20190085084; SEQ ID NO: 390 | 7382 |
| CII5216 | VR | PRT | WO2016164637; SEQ ID NO: 390 | 7383 |
| CII5217 | VR | PRT | US20190085084; SEQ ID NO: 376 | 7384 |
| CII5218 | VR | PRT | US20190085084; SEQ ID NO: 376 | 7385 |
| CII5219 | VR | PRT | WO2016164637; SEQ ID NO: 376 | 7386 |
| CII5220 | VR | PRT | US20190085084; SEQ ID NO: 398 | 7387 |
| CII5221 | VR | PRT | US20190085084; SEQ ID NO: 398 | 7388 |
| CII5222 | VR | PRT | WO2016164637; SEQ ID NO: 398 | 7389 |
| CII5223 | VR | PRT | US20190085084; SEQ ID NO: 392 | 7390 |
| CII5224 | VR | PRT | US20190085084; SEQ ID NO: 392 | 7391 |
| CII5225 | VR | PRT | WO2016164637; SEQ ID NO: 392 | 7392 |
| CII5226 | VR | PRT | US20190085084; SEQ ID NO: 372 | 7393 |
| CII5227 | VR | PRT | WO2016164637; SEQ ID NO: 372 | 7394 |
| CII5228 | VR | PRT | US20190085084; SEQ ID NO: 402 | 7395 |
| CII5229 | VR | PRT | WO2016164637; SEQ ID NO: 402 | 7396 |
| CII5230 | VR | PRT | US20190085084; SEQ ID NO: 380 | 7397 |
| CII5231 | VR | PRT | US20190085084; SEQ ID NO: 380 | 7398 |
| CII5232 | VR | PRT | WO2016164637; SEQ ID NO: 380 | 7399 |
| CII5233 | VR | PRT | US20190085084; SEQ ID NO: 400 | 7400 |
| CII5234 | VR | PRT | US20190085084; SEQ ID NO: 400 | 7401 |
| CII5235 | VR | PRT | WO2016164637; SEQ ID NO: 400 | 7402 |
| CII5236 | VR | PRT | US20190085084; SEQ ID NO: 369 | 7403 |
| CII5237 | VR | PRT | WO2016164637; SEQ ID NO: 369 | 7404 |
| CII5238 | VR | PRT | US20190085084; SEQ ID NO: 365 | 7405 |
| CII5239 | VR | PRT | US20190085084; SEQ ID NO: 365 | 7406 |
| CII5240 | VR | PRT | WO2016164637; SEQ ID NO: 365 | 7407 |
| CII5241 | VR | PRT | US20190085084; SEQ ID NO: 363 | 7408 |
| CII5242 | VR | PRT | US20190085084; SEQ ID NO: 363 | 7409 |
| CII5243 | VR | PRT | WO2016164637; SEQ ID NO: 363 | 7410 |
| CII5244 | VR | PRT | US20190085084; SEQ ID NO: 361 | 7411 |
| CII5245 | VR | PRT | US20190085084; SEQ ID NO: 361 | 7412 |
| CII5246 | VR | PRT | WO2016164637; SEQ ID NO: 361 | 7413 |
| CII5247 | VR | PRT | US20190085084; SEQ ID NO: 479 | 7414 |
| CII5248 | VR | PRT | US20190085084; SEQ ID NO: 479 | 7415 |
| CII5249 | VR | PRT | WO2016164637; SEQ ID NO: 479 | 7416 |
| CII5250 | VR | PRT | US20190085084; SEQ ID NO: 630 | 7417 |
| CII5251 | VR | PRT | WO2016164637; SEQ ID NO: 630 | 7418 |
| CII5252 | VR | PRT | US20190085084; SEQ ID NO: 689 | 7419 |
| CII5253 | VR | PRT | WO2016164637; SEQ ID NO: 689 | 7420 |
| CII5254 | VR | PRT | US20190085084; SEQ ID NO: 692 | 7421 |
| CII5255 | VR | PRT | WO2016164637; SEQ ID NO: 692 | 7422 |
| CII5256 | VR | PRT | US20190085084; SEQ ID NO: 686 | 7423 |
| CII5257 | VR | PRT | WO2016164637; SEQ ID NO: 686 | 7424 |
| CII5258 | VR | PRT | US20190085084; SEQ ID NO: 687 | 7425 |
| CII5259 | VR | PRT | WO2016164637; SEQ ID NO: 687 | 7426 |
| CII5260 | VR | PRT | US20190085084; SEQ ID NO: 473 | 7427 |
| CII5261 | VR | PRT | WO2016164637; SEQ ID NO: 473 | 7428 |
| CII5262 | VR | PRT | US20190085084; SEQ ID NO: 465 | 7429 |
| CII5263 | VR | PRT | US20190085084; SEQ ID NO: 465 | 7430 |
| CII5264 | VR | PRT | WO2016164637; SEQ ID NO: 465 | 7431 |
| CII5265 | VR | PRT | US20190085084; SEQ ID NO: 691 | 7432 |
| CII5266 | VR | PRT | WO2016164637; SEQ ID NO: 691 | 7433 |
| CII5267 | VR | PRT | US20190085084; SEQ ID NO: 477 | 7434 |
| CII5268 | VR | PRT | US20190085084; SEQ ID NO: 477 | 7435 |
| CII5269 | VR | PRT | WO2016164637; SEQ ID NO: 477 | 7436 |
| CII5270 | VR | PRT | US20190085084; SEQ ID NO: 471 | 7437 |
| CII5271 | VR | PRT | US20190085084; SEQ ID NO: 471 | 7438 |
| CII5272 | VR | PRT | WO2016164637; SEQ ID NO: 471 | 7439 |
| CII5273 | VR | PRT | US20190085084; SEQ ID NO: 475 | 7440 |
| CII5274 | VR | PRT | WO2016164637; SEQ ID NO: 475 | 7441 |
| CII5275 | VR | PRT | US20190085084; SEQ ID NO: 685 | 7442 |
| CII5276 | VR | PRT | WO2016164637; SEQ ID NO: 685 | 7443 |
| CII5277 | VR | PRT | US20190085084; SEQ ID NO: 688 | 7444 |
| CII5278 | VR | PRT | WO2016164637; SEQ ID NO: 688 | 7445 |
| CII5279 | VR | PRT | US20190085084; SEQ ID NO: 690 | 7446 |
| CII5280 | VR | PRT | WO2016164637; SEQ ID NO: 690 | 7447 |
| CII5281 | VR | PRT | US20190085084; SEQ ID NO: 428 | 7448 |
| CII5282 | VR | PRT | US20190085084; SEQ ID NO: 428 | 7449 |
| CII5283 | VR | PRT | WO2016164637; SEQ ID NO: 428 | 7450 |
| CII5284 | VR | PRT | US20190085084; SEQ ID NO: 437 | 7451 |
| CII5285 | VR | PRT | WO2016164637; SEQ ID NO: 437 | 7452 |
| CII5286 | VR | PRT | US20190085084; SEQ ID NO: 440 | 7453 |
| CII5287 | VR | PRT | WO2016164637; SEQ ID NO: 440 | 7454 |
| CII5288 | VR | PRT | US20190085084; SEQ ID NO: 426 | 7455 |
| CII5289 | VR | PRT | WO2016164637; SEQ ID NO: 426 | 7456 |
| CII5290 | VR | PRT | US20190085084; SEQ ID NO: 422 | 7457 |
| CII5291 | VR | PRT | US20190085084; SEQ ID NO: 422 | 7458 |
| CII5292 | VR | PRT | WO2016164637; SEQ ID NO: 422 | 7459 |
| CII5293 | VR | PRT | US20190085084; SEQ ID NO: 433 | 7460 |
| CII5294 | VR | PRT | US20190085084; SEQ ID NO: 433 | 7461 |
| CII5295 | VR | PRT | WO2016164637; SEQ ID NO: 433 | 7462 |
| CII5296 | VR | PRT | WO2016164637; SEQ ID NO: 431 | 7463 |
| CII5297 | VR | PRT | US20190085084; SEQ ID NO: 680 | 7464 |
| CII5298 | VR | PRT | WO2016164637; SEQ ID NO: 680 | 7465 |
| CII5299 | VR | PRT | US20190085084; SEQ ID NO: 679 | 7466 |
| CII5300 | VR | PRT | WO2016164637; SEQ ID NO: 679 | 7467 |
| CII5301 | VR | PRT | US20190085084; SEQ ID NO: 459 | 7468 |
| CII5302 | VR | PRT | US20190085084; SEQ ID NO: 459 | 7469 |
| CII5303 | VR | PRT | WO2016164637; SEQ ID NO: 459 | 7470 |
| CII5304 | VR | PRT | US20190085084; SEQ ID NO: 461 | 7471 |
| CII5305 | VR | PRT | US20190085084; SEQ ID NO: 461 | 7472 |
| CII5306 | VR | PRT | WO2016164637; SEQ ID NO: 461 | 7473 |
| CII5307 | VR | PRT | US20190085084; SEQ ID NO: 444 | 7474 |
| CII5308 | VR | PRT | US20190085084; SEQ ID NO: 444 | 7475 |
| CII5309 | VR | PRT | WO2016164637; SEQ ID NO: 444 | 7476 |
| CII5310 | VR | PRT | US20190085084; SEQ ID NO: 442 | 7477 |
| CII5311 | VR | PRT | US20190085084; SEQ ID NO: 442 | 7478 |
| CII5312 | VR | PRT | WO2016164637; SEQ ID NO: 442 | 7479 |
| CII5313 | VR | PRT | US20190085084; SEQ ID NO: 452 | 7480 |
| CII5314 | VR | PRT | US20190085084; SEQ ID NO: 452 | 7481 |
| CII5315 | VR | PRT | WO2016164637; SEQ ID NO: 452 | 7482 |
| CII5316 | VR | PRT | US20190085084; SEQ ID NO: 448 | 7483 |
| CII5317 | VR | PRT | US20190085084; SEQ ID NO: 448 | 7484 |
| CII5318 | VR | PRT | WO2016164637; SEQ ID NO: 448 | 7485 |
| CII5319 | VR | PRT | US20190085084; SEQ ID NO: 454 | 7486 |
| CII5320 | VR | PRT | WO2016164637; SEQ ID NO: 454 | 7487 |
| CII5321 | VR | PRT | US20190085084; SEQ ID NO: 456 | 7488 |
| CII5322 | VR | PRT | US20190085084; SEQ ID NO: 456 | 7489 |
| CII5323 | VR | PRT | WO2016164637; SEQ ID NO: 456 | 7490 |
| CII5324 | VR | PRT | US20190085084; SEQ ID NO: 414 | 7491 |
| CII5325 | VR | PRT | WO2016164637; SEQ ID NO: 414 | 7492 |
| CII5326 | VR | PRT | US20190085084; SEQ ID NO: 412 | 7493 |
| CII5327 | VR | PRT | US20190085084; SEQ ID NO: 412 | 7494 |
| CII5328 | VR | PRT | WO2016164637; SEQ ID NO: 412 | 7495 |
| CII5329 | VR | PRT | US20190085084; SEQ ID NO: 410 | 7496 |
| CII5330 | VR | PRT | WO2016164637; SEQ ID NO: 410 | 7497 |
| CII5331 | VR | PRT | US20190085084; SEQ ID NO: 408 | 7498 |
| CII5332 | VR | PRT | US20190085084; SEQ ID NO: 408 | 7499 |
| CII5333 | VR | PRT | WO2016164637; SEQ ID NO: 408 | 7500 |
| CII5334 | VR | PRT | US20190085084; SEQ ID NO: 651 | 7501 |
| CII5335 | VR | PRT | WO2016164637; SEQ ID NO: 651 | 7502 |
| CII5336 | VR | PRT | US20190085084; SEQ ID NO: 673 | 7503 |
| CII5337 | VR | PRT | WO2016164637; SEQ ID NO: 673 | 7504 |
| CII5338 | VR | PRT | US20190085084; SEQ ID NO: 625 | 7505 |
| CII5339 | VR | PRT | WO2016164637; SEQ ID NO: 625 | 7506 |
| CII5340 | VR | PRT | US20190085084; SEQ ID NO: 613 | 7507 |
| CII5341 | VR | PRT | WO2016164637; SEQ ID NO: 613 | 7508 |
| CII5342 | VR | PRT | US20190085084; SEQ ID NO: 611 | 7509 |
| CII5343 | VR | PRT | WO2016164637; SEQ ID NO: 611 | 7510 |
| CII5344 | VR | PRT | US20190085084; SEQ ID NO: 604 | 7511 |
| CII5345 | VR | PRT | WO2016164637; SEQ ID NO: 604 | 7512 |
| CII5346 | VR | PRT | US20190085084; SEQ ID NO: 609 | 7513 |
| CII5347 | VR | PRT | WO2016164637; SEQ ID NO: 609 | 7514 |
| CII5348 | VR | PRT | WO2016164637; SEQ ID NO: 606 | 7515 |
| CII5349 | VR | PRT | US20190085084; SEQ ID NO: 617 | 7516 |
| CII5350 | VR | PRT | WO2016164637; SEQ ID NO: 617 | 7517 |
| CII5351 | VR | PRT | US20190085084; SEQ ID NO: 610 | 7518 |
| CII5352 | VR | PRT | WO2016164637; SEQ ID NO: 610 | 7519 |
| CII5353 | VR | PRT | US20190085084; SEQ ID NO: 614 | 7520 |
| CII5354 | VR | PRT | WO2016164637; SEQ ID NO: 614 | 7521 |
| CII5355 | VR | PRT | US20190085084; SEQ ID NO: 608 | 7522 |
| CII5356 | VR | PRT | WO2016164637; SEQ ID NO: 608 | 7523 |
| CII5357 | VR | PRT | US20190085084; SEQ ID NO: 618 | 7524 |
| CII5358 | VR | PRT | WO2016164637; SEQ ID NO: 618 | 7525 |
| CII5359 | VR | PRT | US20190085084; SEQ ID NO: 612 | 7526 |
| CII5360 | VR | PRT | WO2016164637; SEQ ID NO: 612 | 7527 |
| CII5361 | VR | PRT | US20190085084; SEQ ID NO: 615 | 7528 |
| CII5362 | VR | PRT | WO2016164637; SEQ ID NO: 615 | 7529 |
| CII5363 | VR | PRT | US20190085084; SEQ ID NO: 675 | 7530 |
| CII5364 | VR | PRT | WO2016164637; SEQ ID NO: 675 | 7531 |
| CII5365 | VR | PRT | US20190085084; SEQ ID NO: 327 | 7532 |
| CII5366 | VR | PRT | WO2016164637; SEQ ID NO: 327 | 7533 |
| CII5367 | VR | PRT | US20190085084; SEQ ID NO: 325 | 7534 |
| CII5368 | VR | PRT | WO2016164637; SEQ ID NO: 325 | 7535 |
| CII5369 | VR | PRT | US20190085084; SEQ ID NO: 322 | 7536 |
| CII5370 | VR | PRT | US20190085084; SEQ ID NO: 322 | 7537 |
| CII5371 | VR | PRT | WO2016164637; SEQ ID NO: 322 | 7538 |
| CII5372 | VR | PRT | US20190085084; SEQ ID NO: 320 | 7539 |
| CII5373 | VR | PRT | WO2016164637; SEQ ID NO: 320 | 7540 |
| CII5374 | VR | PRT | US20190085084; SEQ ID NO: 318 | 7541 |
| CII5375 | VR | PRT | US20190085084; SEQ ID NO: 318 | 7542 |
| CII5376 | VR | PRT | WO2016164637; SEQ ID NO: 318 | 7543 |
| CII5377 | VR | PRT | US20190085084; SEQ ID NO: 616 | 7544 |
| CII5378 | VR | PRT | WO2016164637; SEQ ID NO: 616 | 7545 |
| CII5379 | VR | PRT | US20190085084; SEQ ID NO: 644 | 7546 |
| CII5380 | VR | PRT | WO2016164637; SEQ ID NO: 644 | 7547 |
| CII5381 | VR | PRT | US20190085084; SEQ ID NO: 655 | 7548 |
| CII5382 | VR | PRT | WO2016164637; SEQ ID NO: 655 | 7549 |
| CII5383 | VR | PRT | US20190085084; SEQ ID NO: 646 | 7550 |
| CII5384 | VR | PRT | WO2016164637; SEQ ID NO: 646 | 7551 |
| CII5385 | VR | PRT | US20190085084; SEQ ID NO: 662 | 7552 |
| CII5386 | VR | PRT | WO2016164637; SEQ ID NO: 662 | 7553 |
| CII5387 | VR | PRT | US20190085084; SEQ ID NO: 652 | 7554 |
| CII5388 | VR | PRT | WO2016164637; SEQ ID NO: 652 | 7555 |
| CII5389 | VR | PRT | US20190085084; SEQ ID NO: 656 | 7556 |
| CII5390 | VR | PRT | WO2016164637; SEQ ID NO: 656 | 7557 |
| CII5391 | VR | PRT | US20190085084; SEQ ID NO: 641 | 7558 |
| CII5392 | VR | PRT | WO2016164637; SEQ ID NO: 641 | 7559 |
| CII5393 | VR | PRT | US20190085084; SEQ ID NO: 657 | 7560 |
| CII5394 | VR | PRT | WO2016164637; SEQ ID NO: 657 | 7561 |
| CII5395 | VR | PRT | US20190085084; SEQ ID NO: 649 | 7562 |
| CII5396 | VR | PRT | WO2016164637; SEQ ID NO: 649 | 7563 |
| CII5397 | VR | PRT | WO2016164637; SEQ ID NO: 648 | 7564 |
| CII5398 | VR | PRT | US20190085084; SEQ ID NO: 650 | 7565 |
| CII5399 | VR | PRT | WO2016164637; SEQ ID NO: 650 | 7566 |
| CII5400 | VR | PRT | US20190085084; SEQ ID NO: 659 | 7567 |
| CII5401 | VR | PRT | WO2016164637; SEQ ID NO: 659 | 7568 |
| CII5402 | VR | PRT | US20190085084; SEQ ID NO: 647 | 7569 |
| CII5403 | VR | PRT | WO2016164637; SEQ ID NO: 647 | 7570 |
| CII5404 | VR | PRT | US20190085084; SEQ ID NO: 661 | 7571 |
| CII5405 | VR | PRT | WO2016164637; SEQ ID NO: 661 | 7572 |
| CII5406 | VR | PRT | US20190085084; SEQ ID NO: 643 | 7573 |
| CII5407 | VR | PRT | WO2016164637; SEQ ID NO: 643 | 7574 |
| CII5408 | VR | PRT | US20190085084; SEQ ID NO: 645 | 7575 |
| CII5409 | VR | PRT | WO2016164637; SEQ ID NO: 645 | 7576 |
| CII5410 | VR | PRT | US20190085084; SEQ ID NO: 654 | 7577 |
| CII5411 | VR | PRT | WO2016164637; SEQ ID NO: 654 | 7578 |
| CII5412 | VR | PRT | US20190085084; SEQ ID NO: 658 | 7579 |
| CII5413 | VR | PRT | WO2016164637; SEQ ID NO: 658 | 7580 |
| CII5414 | VR | PRT | US20190085084; SEQ ID NO: 665 | 7581 |
| CII5415 | VR | PRT | WO2016164637; SEQ ID NO: 665 | 7582 |
| CII5416 | VR | PRT | US20190085084; SEQ ID NO: 669 | 7583 |
| CII5417 | VR | PRT | WO2016164637; SEQ ID NO: 669 | 7584 |
| CII5418 | VR | PRT | US20190085084; SEQ ID NO: 639 | 7585 |
| CII5419 | VR | PRT | WO2016164637; SEQ ID NO: 639 | 7586 |
| CII5420 | VR | PRT | US20190085084; SEQ ID NO: 664 | 7587 |
| CII5421 | VR | PRT | WO2016164637; SEQ ID NO: 664 | 7588 |
| CII5422 | VR | PRT | US20190085084; SEQ ID NO: 668 | 7589 |
| CII5423 | VR | PRT | WO2016164637; SEQ ID NO: 668 | 7590 |
| CII5424 | VR | PRT | US20190085084; SEQ ID NO: 666 | 7591 |
| CII5425 | VR | PRT | WO2016164637; SEQ ID NO: 666 | 7592 |
| CII5426 | VR | PRT | US20190085084; SEQ ID NO: 640 | 7593 |
| CII5427 | VR | PRT | WO2016164637; SEQ ID NO: 640 | 7594 |
| CII5428 | VR | PRT | US20190085084; SEQ ID NO: 629 | 7595 |
| CII5429 | VR | PRT | WO2016164637; SEQ ID NO: 629 | 7596 |
| CII5430 | VR | PRT | US20190085084; SEQ ID NO: 626 | 7597 |
| CII5431 | VR | PRT | WO2016164637; SEQ ID NO: 626 | 7598 |
| CII5432 | VR | PRT | US20190085084; SEQ ID NO: 627 | 7599 |
| CII5433 | VR | PRT | WO2016164637; SEQ ID NO: 627 | 7600 |
| CII5434 | VR | PRT | US20190085084; SEQ ID NO: 624 | 7601 |
| CII5435 | VR | PRT | WO2016164637; SEQ ID NO: 624 | 7602 |
| CII5436 | VR | PRT | US20190085084; SEQ ID NO: 631 | 7603 |
| CII5437 | VR | PRT | WO2016164637; SEQ ID NO: 631 | 7604 |
| CII5438 | VR | PRT | US20190085084; SEQ ID NO: 632 | 7605 |
| CII5439 | VR | PRT | WO2016164637; SEQ ID NO: 632 | 7606 |
| CII5440 | VR | PRT | US20190085084; SEQ ID NO: 633 | 7607 |
| CII5441 | VR | PRT | WO2016164637; SEQ ID NO: 633 | 7608 |
| CII5442 | VR | PRT | US20190085084; SEQ ID NO: 634 | 7609 |
| CII5443 | VR | PRT | WO2016164637; SEQ ID NO: 634 | 7610 |
| CII5444 | VR | PRT | US20190085084; SEQ ID NO: 635 | 7611 |
| CII5445 | VR | PRT | WO2016164637; SEQ ID NO: 635 | 7612 |
| CII5446 | VR | PRT | US20190085084; SEQ ID NO: 636 | 7613 |
| CII5447 | VR | PRT | WO2016164637; SEQ ID NO: 636 | 7614 |
| CII5448 | VR | PRT | US20190085084; SEQ ID NO: 637 | 7615 |
| CII5449 | VR | PRT | WO2016164637; SEQ ID NO: 637 | 7616 |
| CII5450 | VR | PRT | US20190085084; SEQ ID NO: 663 | 7617 |
| CII5451 | VR | PRT | WO2016164637; SEQ ID NO: 663 | 7618 |
| CII5452 | VR | PRT | US20190085084; SEQ ID NO: 619 | 7619 |
| CII5453 | VR | PRT | WO2016164637; SEQ ID NO: 619 | 7620 |
| CII5454 | VR | PRT | US20190085084; SEQ ID NO: 621 | 7621 |
| CII5455 | VR | PRT | WO2016164637; SEQ ID NO: 621 | 7622 |
| CII5456 | VR | PRT | US20190085084; SEQ ID NO: 329 | 7623 |
| CII5457 | VR | PRT | US20190085084; SEQ ID NO: 329 | 7624 |
| CII5458 | VR | PRT | WO2016164637; SEQ ID NO: 329 | 7625 |
| CII5459 | VR | PRT | US20190085084; SEQ ID NO: 331 | 7626 |
| CII5460 | VR | PRT | WO2016164637; SEQ ID NO: 331 | 7627 |
| CII5461 | VR | PRT | US20190085084; SEQ ID NO: 338 | 7628 |
| CII5462 | VR | PRT | US20190085084; SEQ ID NO: 338 | 7629 |
| CII5463 | VR | PRT | WO2016164637; SEQ ID NO: 338 | 7630 |
| CII5464 | VR | PRT | US20190085084; SEQ ID NO: 333 | 7631 |
| CII5465 | VR | PRT | US20190085084; SEQ ID NO: 333 | 7632 |
| CII5466 | VR | PRT | WO2016164637; SEQ ID NO: 333 | 7633 |
| CII5467 | VR | PRT | US20190085084; SEQ ID NO: 638 | 7634 |
| CII5468 | VR | PRT | WO2016164637; SEQ ID NO: 638 | 7635 |
| CII5469 | VR | PRT | US20190085084; SEQ ID NO: 622 | 7636 |
| CII5470 | VR | PRT | WO2016164637; SEQ ID NO: 622 | 7637 |
| CII5471 | VR | PRT | US20190085084; SEQ ID NO: 623 | 7638 |
| CII5472 | VR | PRT | WO2016164637; SEQ ID NO: 623 | 7639 |
| CII5473 | VR | PRT | US20190085084; SEQ ID NO: 607 | 7640 |
| CII5474 | VR | PRT | WO2016164637; SEQ ID NO: 607 | 7641 |
| CII5475 | VR | PRT | US20190085084; SEQ ID NO: 620 | 7642 |
| CII5476 | VR | PRT | WO2016164637; SEQ ID NO: 620 | 7643 |
| CII5477 | VR | PRT | WO2016164637; SEQ ID NO: 342 | 7644 |
| CII5478 | VR | PRT | US20190085084; SEQ ID NO: 605 | 7645 |
| CII5479 | VR | PRT | WO2016164637; SEQ ID NO: 605 | 7646 |
| CII5480 | VR | PRT | US20190085084; SEQ ID NO: 628 | 7647 |
| CII5481 | VR | PRT | WO2016164637; SEQ ID NO: 628 | 7648 |
| CII5482 | VR | PRT | US20190085084; SEQ ID NO: 642 | 7649 |
| CII5483 | VR | PRT | WO2016164637; SEQ ID NO: 642 | 7650 |
| CII5484 | VR | PRT | US20190085084; SEQ ID NO: 677 | 7651 |
| CII5485 | VR | PRT | WO2016164637; SEQ ID NO: 677 | 7652 |
| CII5486 | VR | PRT | US20190085084; SEQ ID NO: 678 | 7653 |
| CII5487 | VR | PRT | WO2016164637; SEQ ID NO: 678 | 7654 |
| CII5488 | VR | PRT | US20190085084; SEQ ID NO: 672 | 7655 |
| CII5489 | VR | PRT | WO2016164637; SEQ ID NO: 672 | 7656 |
| CII5490 | VR | PRT | US20190085084; SEQ ID NO: 676 | 7657 |
| CII5491 | VR | PRT | WO2016164637; SEQ ID NO: 676 | 7658 |
| CII5492 | VR | PRT | US20190085084; SEQ ID NO: 671 | 7659 |
| CII5493 | VR | PRT | WO2016164637; SEQ ID NO: 671 | 7660 |
| CII5494 | VR | PRT | US20190085084; SEQ ID NO: 353 | 7661 |
| CII5495 | VR | PRT | WO2016164637; SEQ ID NO: 353 | 7662 |
| CII5496 | VR | PRT | US20190085084; SEQ ID NO: 345 | 7663 |
| CII5497 | VR | PRT | US20190085084; SEQ ID NO: 345 | 7664 |
| CII5498 | VR | PRT | WO2016164637; SEQ ID NO: 345 | 7665 |
| CII5499 | VR | PRT | US20190085084; SEQ ID NO: 351 | 7666 |
| CII5500 | VR | PRT | WO2016164637; SEQ ID NO: 351 | 7667 |
| CII5501 | VR | PRT | US20190085084; SEQ ID NO: 357 | 7668 |
| CII5502 | VR | PRT | WO2016164637; SEQ ID NO: 357 | 7669 |
| CII5503 | VR | PRT | US20190085084; SEQ ID NO: 674 | 7670 |
| CII5504 | VR | PRT | WO2016164637; SEQ ID NO: 674 | 7671 |
| CII5505 | VR | PRT | US20190085084; SEQ ID NO: 670 | 7672 |
| CII5506 | VR | PRT | WO2016164637; SEQ ID NO: 670 | 7673 |
| CII5507 | VR | PRT | US20190085084; SEQ ID NO: 349 | 7674 |
| CII5508 | VR | PRT | WO2016164637; SEQ ID NO: 349 | 7675 |
| CII5509 | VR | PRT | US20190085084; SEQ ID NO: 355 | 7676 |
| CII5510 | VR | PRT | WO2016164637; SEQ ID NO: 355 | 7677 |
| CII5511 | VR | PRT | US20190085084; SEQ ID NO: 653 | 7678 |
| CII5512 | VR | PRT | WO2016164637; SEQ ID NO: 653 | 7679 |
| CII5513 | VR | PRT | US20190085084; SEQ ID NO: 660 | 7680 |
| CII5514 | VR | PRT | WO2016164637; SEQ ID NO: 660 | 7681 |

In some embodiments, the payload region of the AAV particle comprises a nucleic acid sequence with at least 50% identity to one or more nucleic acid sequences listed in Table 4, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 4, or variants or fragments thereof.

In some embodiments, the payload nucleic acid sequence has 90% identity to one or more of the nucleic acid sequences listed in Table 4, or variants or fragments thereof.

In some embodiments, the payload nucleic acid sequence has 91% identity to one or more of the nucleic acid sequences listed in Table 4, or variants or fragments thereof.

In some embodiments, the payload nucleic acid sequence has 92% identity to one or more of the nucleic acid sequences listed in Table 4, or variants or fragments thereof.

In some embodiments, the payload nucleic acid sequence has 93% identity to one or more of the nucleic acid sequences listed in Table 4, or variants or fragments thereof.

In some embodiments, the payload nucleic acid sequence has 94% identity to one or more of the nucleic acid sequences listed in Table 4, or variants or fragments thereof.

In some embodiments, the payload nucleic acid sequence has 95% identity to one or more of the nucleic acid sequences listed in Table 4, or variants or fragments thereof.

In some embodiments, the payload nucleic acid sequence has 96% identity to one or more of the nucleic acid sequences listed in Table 4, or variants or fragments thereof.

In some embodiments, the payload nucleic acid sequence has 97% identity to one or more of the nucleic acid sequences listed in Table 4, or variants or fragments thereof.

In some embodiments, the payload nucleic acid sequence has 98% identity to one or more of the nucleic acid sequences listed in Table 4, or variants or fragments thereof.

In some embodiments, the payload nucleic acid sequence has 99% identity to one or more of the nucleic acid sequences listed in Table 4, or variants or fragments thereof.

In some embodiments, the payload nucleic acid sequence has 100% identity to one or more of the nucleic acid sequences listed in Table 4, or variants or fragments thereof.

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 4, or variants or fragments thereof. As another non-limiting example, the antibody may be one or more of the heavy chain sequences listed in Table 4. As a non-limiting example, the antibody may be one or more of the light chain sequences listed in Table 4, or variants or fragments thereof.

In some embodiments, the payload region of the AAV particle comprises a nucleic acid sequence encoding a polypeptide comprising a heavy chain and alight chain sequence listed in Table 4, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Table 4, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 4, one or more linkers from Table 2 and a heavy chain sequence from Table 4.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 4, one or more linkers from Table 2, and alight chain sequence from Table 4.

In some embodiments, the payload region comprises a nucleic acid sequence encoding a single heavy chain. As a non-limiting example, the heavy chain is an amino acid sequence or fragment thereof described in Table 4.

Shown in Table 4 are a listing of antibodies and their polynucleotides and/or polypeptides sequences. These sequences may be encoded by or included in the AAV particles of the present disclosure. Variants or fragments of the antibody sequences described in Table 4 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 4. CDRs of the antibodies encoded by the viral genomes of the present disclosure may be 50%, 60%, 70%, 80%, 90%, 95% identical to CDRs listed in or incorporated in the sequences of Table 4. Methods of determining CDRs are well known in the art and are described herein. Payload regions may encode antibody variants with one or more heavy chain variable domain (V_{H}) or light chain variable domain (V_{L}) derived from the antibody sequences in Table 4. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any cancer and immunoinflammatory disease-associated antibodies, not limited to those described in Table 4, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the cancer and immunoinflammatory disease-associated antibodies as described in International Publication Number WO2016059622, WO2017046053, WO2017046658, WO2017046676, WO2017046774, WO2017046776, WO2017048593, WO2017048614, WO2017048629, WO2017049024, WO2017049035, WO2017049038, WO2017049139, WO2017049149, WO2017049251, WO2017049266, WO2017049296, WO2017049452, WO2017050729, WO2017050889, WO2017051002, WO2017051888, WO2017052241, WO2017052679, WO2017053170, WO2017053250, WO2017053423, WO2017053469, WO2017053556, WO2017053703, WO2017053705, WO2017053748, WO2017053807, WO2017053856, WO2017053889, WO2017053905, WO2017053932, WO2017054089, WO2017054141, WO2017054646, WO2017055273, WO2017055291, WO2017055314, WO2017055318, WO2017055328, WO2017055385, WO2017055388, WO2017055389, WO2017055391, WO2017055392, WO2017055393, WO2017055395, WO2017055398, WO2017055404, WO2017055443, WO2017055521, WO2017055547, WO2017055612, WO2017055613, WO2017055614, WO2017055617, WO2017058115, WO2017058771, WO2017058780, WO2017058866, WO2017059095, WO2017059196, WO2017059243, WO2017059252, WO2017059289, WO2017059380, WO2017059551, WO2017059557, WO2017059878, WO2017059900, WO2017060144, WO2017060201, WO2017060247, WO2017060300, WO2017060322, WO2017060504, WO2017060857, WO2017061602, WO2017062016, WO2017062271, WO2017062456, WO2017062496, WO2017062615, WO2017062619, WO2017062649, WO2017062672, WO2017062682, WO2017062748, WO2017062792, WO2017062820, WO2017062830, WO2017062888, WO2017062952, WO2017062966, WO2017063593, WO2017064043, WO2017064221, WO2017064564, WO2017064716, WO2017065203, WO2017065484, WO2017065493, WO2017065837, WO2017066122, WO2017066136, WO2017066714, WO2017066760, WO2017067042, WO2017067043, WO2017067480, WO2017068186, WO2017068360, WO2017068472, WO2017069628, WO2017070170, WO2017070364, WO2017070395, WO2017070423, WO2017070456, WO2017070460, WO2017070475, WO2017070476, WO2017070527, WO2017070554, WO2017070561, WO2017070567, WO2017070613, WO2017070616, WO2017070622, WO2017070623, WO2017070624, WO2017070626, WO2017070649, WO2017070654, WO2017070943, WO2017071625, WO2017072150, WO2017072196, WO2017072207, WO2017072208, WO2017072361, WO2017072366, WO2017072662, WO2017072757, WO2017073619, WO2017073981, WO2017074013, WO2017074074, WO2017074428, WO2017074878, WO2017075052, WO2017075119, WO2017075124, WO2017075325, WO2017075432, WO2017075484, WO2017075533, WO2017075615, WO2017076308, WO2017076878, WO2017076916, WO2017077047, WO2017077085, WO2017077382, WO2017077391, WO2017078839, WO2017079112, WO2017079115, WO2017079116, WO2017079117, WO2017079121, WO2017079150, WO2017079165, WO2017079369, WO2017079443, WO2017079448, WO2017079479, WO2017079520, WO2017079591, WO2017079705, WO2017079831, WO2017079832, WO2017079833, WO2017079834, WO2017079835, WO2017080850, WO2017081066, WO2017081211, WO2017081265, WO2017081320, WO2017082214, WO2017083296, WO2017083306, WO2017083314, WO2017083441, WO2017083451, WO2017083488, WO2017083511, WO2017083515, WO2017083582, WO2017083627, WO2017083750, WO2017084078, WO2017084495, WO2017085166, WO2017085172, WO2017085212, WO2017085471, WO2017085693, WO2017086627, WO2017087280, WO2017087547, WO2017087587, WO2017087588, WO2017087589, WO2017087599, WO2017087678, WO2017087800, WO2017087843, WO2017087921 WO2017088028, WO2017088734, WO2017088782, WO2017088821, WO2017088974, WO2017089334, WO2017089447, WO2017089618, WO2017089895, WO2017091429, WO2017091487, WO2017091577, WO2017091580, WO2017091615, WO2017091683, WO217091719, WO2017091850, WO2017092645, WO2017093402, WO2017093404, WO2017093406, WO2017093408, WO2017093410, WO2017093448, WO2017093478, WO2017093844, WO2017093845, WO2017093942, WO2017093947, WO2017095088, WO2017095267, WO2017095486, WO2017095487, WO2017095805, WO2017095808, WO2017095823, WO2017095875, WO2017095918, WO2017095939, WO2017096017, WO2017096026, WO2017096051, WO2017096120, WO2017096163, WO2017096179, WO2017096182, WO2017096189, WO2017096221, WO2017096276, WO2017096281, WO2017096327, WO2017096329, WO2017096361, WO2017096397, WO2017096486, WO2017097723, WO2017097990, WO2017099362, WO2017099712, WO2017100289, WO2017100305, WO2017100372, WO2017100467, WO2017100540, WO2017100597, WO2017100670, WO2017101863, WO2017102010, WO2017102789, WO2017102833, WO2017103088, WO2017103895, WO2017104779, WO2017106061, WO2017106129, WO2017106196, WO2017106236, WO2017106346, WO2017106352, WO2017106372, WO2017106383, WO2017106453, WO2017106462, WO2017106501, WO2017106508, WO2017106566, WO2017106609, WO2017106643, WO2017106656, WO2017106684, WO201710610, WO2017106859, WO2017107885, WO2017107973, WO2017109290, WO2017109496, WO2017109721, WO2017112536, WO2017112741, WO2017112762, WO2017112775, WO2017112803, WO2017112811, WO2017112829, WO2017112877, WO2017112944, WO217112954, WO2017112955, WO2017112956, WO2017114204, WO2017114401, WO2017114694, WO2017114972, WO2017114973, WO2017114975, WO2017114976, WO2017116064, WO2017116196, WO2017117179, WO2017117202, WO2017117384, WO2017117430, WO2017117464, WO2017118321, WO2017118613, WO2017118675, WO2017118745, WO2017118761, WO2017118841, WO2017119434, WO2017119435, WO2017119811, WO2017120222, WO2017120280, WO2017120344, WO2017120525, WO2017120534, WO2017120557, WO2017120599, WO2017120612, WO2017120613, WO2017120996, WO2017120997, WO2017120998, WO2017121330, WO2017121758, WO2017121771, WO2017121877, WO2017121880, WO2017121905, WO2017122017, WO2017122018, WO2017122019, WO2017122130, WO2017122666, WO2017123556, WO2017123557, WO2017123636, WO2017123644, WO2017123646, WO2017123650, WO2017123673, WO2017123804, WO2017123978, WO2017124001, WO2017124002, WO2017124050, WO2017124110, WO2017125532, WO2017125578, WO2017125601, WO2017125830, WO2017125831, WO2017125871, WO2017125897, WO2017126587, WO217126944, WO2017127468, WO2017127499, WO2017127664, WO2017127702, WO2017127764, WO2017127833, WO2017127933, WO2017128534, WO2017128880, WO2017130076, WO2017130223, WO2017132062, WO2017132457, WO2017132459, WO2017132486, WO2017132555, WO2017132562, WO2017132772, WO2017132827, WO2017133174, WO2017133175, WO2017133540, WO2017133633, WO2017133639, WO2017133640, WO2017133745, WO2017134140, WO2017134158, WO2017134197, WO2017134234, WO2017134292, WO2017134301, WO2017134302, WO2017134305, WO2017134306, WO2017134440, WO2017134592, WO2017135791, WO2017136313, WO2017136350, WO2017136355, WO2017136549, WO2017136562, WO2017136607, WO2017136659, WO2017136693, WO2017136820, WO2017137503, WO2017137542, WO2017137556, WO2017137579, WO2017137613, WO2017137954, WO2017139212, WO2017139587, WO2017139623, WO2017139975, WO2017140256, WO2017142294, WO2017142800, WO2017142928, WO2017143062, WO2017143069, WO2017143073, WO2017143076, WO2017143270, WO2017143451, WO2017143838, WO2017143840, WO2017144621, WO2017144668, WO2017144681, WO2017144896, WO2017144900, WO2017144903, WO2017147247, WO2017147248, WO2017147278, WO2017147293, WO2017147368, WO2017147383, WO2017147538, WO2017147653, WO217147719, WO2017147742, WO2017148424, WO2017148879, WO2017148880, WO2017148889, WO2017149106, WO2017149122, WO2017149143, WO2017149394, WO2017149513, WO2017149515, WO2017149538, WO2017150762, WO2017151000, WO2017151176, WO2017151502, WO2017151818, WO2017151940, WO2017151979, WO2017152076, WO2017152085, WO2017152088, WO2017152102, WO2017153345, WO2017153402, WO2017153433, WO2017153438, WO2017153541, WO2017153567, WO2017154868, WO2017155355, WO2017155996, WO2017156032, WO2017156058, WO2017156298, WO2017156355, WO2017156479, WO2017156488, WO2017156500, WO2017157305, WO2017157895, WO2017157948, WO2017158079, WO2017158084, WO2017158092, WO2017158097, WO2017158101, WO2017158103, WO2017158116, WO2017158337, WO2017158339, WO2017158436, WO2017159287, WO2017159699, WO2017159996, WO2017160587, WO2017160699, WO2017160700, WO2017160725, WO2017160754, WO2017160775, WO2017161169, WO2017161206, WO2017161414, WO2017161976, WO2017162555, WO2017162587, WO2017162663, WO2017162678, WO217162791, WO2017162890, WO2017163049, WO2017163064, WO2017165398, WO2017165460, WO2017165464, WO2017165614, WO2017165683, WO2017165734, WO2017165736, WO2017165742, WO2017165778, WO2017166804, WO2017167967, WO2017167997, WO2017168726, WO2017170597, WO2017170847, WO2017171373, WO2017171535, WO217172260, WO2017172509, WO2017172517, WO2017172518, WO2017172733, WO2017172771, WO2017172990, WO2017173256, WO2017173328, WO2017173349, WO2017173359, WO2017173384, WO2017173410, WO2017173494, WO2017174017, WO2017174568, WO2017174586, WO2017174822, WO2017174823, WO2017174824, WO2017175006, WO2017175018, WO2017175058, WO2017175176, WO2017176007, WO2017176760, WO2017176864, WO2017177013, WO2017177137, WO2017177175, WO2017177179, WO2017177199, WO2017177217, WO2017177333, WO2017177955, WO2017178493, WO2017178562, WO2017178569, WO2017178653, WO2017179862, WO2017180462, WO2017180530, WO2017180555, WO2017180713, WO2017180738, WO2017180813, WO2017180842, WO2017180864, WO2017180904, WO2017180913, WO2017180936, WO2017180993, WO2017181001, WO2017181031, WO2017181034, WO2017181039, WO2017181049, WO2017181098, WO2017181109, WO2017181111, WO2017181119, WO2017181139, WO2017181243, WO2017181420, WO2017182603, WO2017182672, WO2017182705, WO2017183665, WO2017183711, WO2017184201, WO217184562, WO2017184619, WO2017184942, WO2017184973, WO2017185037, WO2017185177, WO2017185492, WO2017186121, WO2017186182, WO2017186853, WO2017186908, WO2017186928, WO2017186950, WO2017187307, WO2017188472, WO2017188570, WO2017189279, WO2017189432, WO2017189588, WO2017189724, WO2017189805, WO2017189827, WO2017189959, WO2017190025, WO2017190100, WO2017191062, WO2017191101, WO2017191108, WO2017191325, WO2017191439, WO2017191476, WO2017191559, WO2017191579, WO2017191627, WO2017191748, WO2017192387, WO2017192470, WO2017192536, WO2017192567, WO2017192589, WO2017192668, WO2017192743, WO2017192867, WO2017192933, WO2017193032, WO2017193059, WO2017193094, WO2017193096, WO2017193097, WO2017193101, WO2017193107, WO2017193956, WO2017194265, WO2017194390, WO2017194438, WO2017194441, WO2017194442, WO2017194555, WO2017194568, WO2017194641, WO2017194646, WO2017194782, WO2017194783, WO2017195749, WO2017196263, WO2017196426, WO2017196663, WO2017196764, WO2017196819, WO2017196847, WO2017196867, WO2017196902, WO2017197098, WO2017197199, WO2017197231, WO2017197234, WO2017197259, WO2017197263, WO2017197265, WO2017197331, WO2017197347, WO2017197376, WO2017197667, WO2017198212, WO217198741, WO2017199250, WO2017200173, WO2017200493, WO2017201131, WO2017201204, WO2017201210, WO2017201325, WO2017201340, WO2017201440, WO2017201442, WO2017201476, WO2017201488, WO2017201493, WO2017201731, WO2017201766, WO2017202387, WO2017202744, WO2017202879, WO2017203426, WO2017203468, WO2017204277, WO2017204606, WO2017205014, WO2017205101, WO2017205216, WO2017205465, WO2017205536, WO2017205538, WO2017205651, WO2017205721, WO2017205726, WO2017205738, WO2017205742, WO2017205745, WO2017205747, WO2017205875, WO2017206621, WO2017206773, WO2017206840, WO2017207477, WO2017207480, WO2017207694, WO2017207739, WO2017207775, WO2017208211, WO2017209351, WO2017210058, WO2017210443, WO2017210485, WO2017210586, WO2017210588, WO2017210621, WO2017210844, WO2017211278, WO2017211313, WO2017211319, WO2017211321, WO2017211900, WO2017211928, WO2017212250, WO2017212442, WO2017212462, WO2017213267, WO2017214089, WO2017214151, WO2017214167, WO2017214170, WO2017214182, WO2017214186, WO217214207, WO2017214282, WO2017214298, WO2017214301, WO2017214335, WO2017214338, WO2017214376, WO2017214456, WO2017214458, WO2017214462, WO2017214547, WO2017214548, WO2017214625, WO2017214706, WO2017215524, WO2017215585, WO2017215590, WO2017216028, WO2017216227, WO2017216324, WO2017216561, WO217216562, WO2017216724, WO2017217128, WO2017217524, WO2017217934, WO2017217985, WO2017218355, WO2017218435, WO2017218515, WO2017218533, WO2017218698, WO2017218824, WO2017218825, WO2017218977, WO2017219025, WO2017219029, WO2017219093, WO2017219687, WO2017219690, WO2017219974, WO2017219995, WO2017220369, WO2017220569, WO2017220645, WO2017220695, WO2017220739, WO2017220988, WO2017220989, WO2017220990, WO2017221128, WO2017221174, WO2017222556, WO2017222593, WO2017223111, WO2017223284, WO2017223286, WO2017223405, WO2018000047, WO2018000324, WO2018001858, WO2018001909, WO2018002031, WO2018002181, WO2018002339, WO2018002952, WO2018004283, WO2018005054, WO2018005226, WO2018005276, WO2018005519, WO2018005556, WO2018005558, WO2018005559, WO2018005682, WO2018005697. WO2018005706, WO2018005904, WO2018006005, WO2018006034, WO2018006092, WO2018006785, WO2018006824, WO2018006882, WO2018007258, WO2018007442, WO2018007592, WO2018007885, WO2018008740, WO2018009499, WO2018009507, WO2018009624, WO2018009811, WO2018009894, WO2018009903, WO2018009921, WO2018010140, WO2018010846, WO2018011421, WO2018011573, WO2018011584, WO2018012952, WO2018013483, WO2018013563, WO2018013585, WO2018013714, WO2018013818, WO2018013917, WO2018013918, WO2018013939, WO2018014001, WO2018014122, WO2018014126, WO2018014260, WO2018014855, WO2018014864, WO2018015340, WO2018015448, WO2018015573, WO2018015880, WO2018016881, WO2018017497, WO2018017604, WO2018017673, WO2018017708, WO2018017714, WO2018017761, WO2018017763, WO2018017786, WO2018017864, WO2018017888, WO2018017889, WO2018017964, WO2018017996, WO2018018031, WO2018018039, WO2018019772, WO2018019897, WO2018019961, WO2018020000, WO2018020273, WO2018020476, WO2018021301, WO2018021972, WO2018022479, WO2018022762, WO2018022786, WO2018023025, WO2018023093, WO2018023100, WO2018023121, WO2018023136, WO2018024237, WO2018024770, WO2018025178, WO2018025982, WO2018026018, WO2018026248, WO2018026249, WO2018026533 WO2018026600, WO2018026722, WO2018026742, WO2018026748, WO2018026819, WO2018026953, WO2018026969, WO2018027025, WO2018027039, WO2018027042, WO2018027084, WO2018027124, WO2018027155, WO2018027203, WO2018027204, WO2018027252, WO2018027329, WO2018029124, WO2018029182, WO2018029284, WO2018029474, WO2018030806, WO2018031258, WO2018031408, WO2018031424, WO2018031454, WO2018031490, WO2018031865, WO2018031887, WO2018032638, WO2018033135, WO2018033749, WO2018033798, WO2018034225, WO2018034226, WO2018034227, WO2018034259, WO2018034301, WO2018034434, WO2018035061, WO2018035084, WO2018035107, WO2018035119, WO2018035210, WO2018035407, WO2018035710, WO2018036243, WO2018036438, WO2018036472, WO2018036473, WO2018036561, WO2018036852, WO2018038583, WO2018038684, WO2018038945, WO2018039020, WO2018039097, WO2018039107, WO2018039180, WO2018039274, WO2018039626, WO2018041067, WO2018041823, WO2018042385, WO2018043311, WO2018044105, WO2018044619, WO2018044948, WO2018045054, WO2018045090, WO2018045110, WO2018045130, WO2018045325, WO2018045379, WO2018046997, WO2018047154, WO2018047894, WO2018048234, WO2018048318, WO2018048975, WO2018049053, WO2018049083, WO2018049118, WO2018049120, WO2018049124, WO2018049130, WO2018049188, WO2018049248, WO2018049474, WO2018050027, WO2018050028, WO2018050039, WO2018050066, WO2018050111, WO2018050733, WO2018050783, WO2018050833, WO2018050852, WO2018050902, WO2018052153, WO2018052503, WO2018052556, WO2018052789, WO2018052818, WO2018053029, WO2018053032, WO2018053106, WO2018053119, WO2018053180, WO2018053270, WO2018053328, WO2018053365, WO2018053401, WO2018053405, WO2018053434, WO2018053468, WO2018053506, WO2018053709, WO2018054240, WO2018054241, WO2018054353, WO2018054484, WO2018054940, WO2018054973, WO2018055503, WO2018056821, WO2018056897, WO2018057303, WO2018057585, WO2018057669, WO2018057735, WO2018057776, WO2018057802, WO2018057823, WO2018057904, WO2018057916, WO2018057955, WO2018057967, WO2018058001, WO2018058002. WO2018058022, WO2018058177, WO2018059117, WO2018059437, WO2018059465, WO2018059502, WO2018060239, WO2018060301, WO2018060351, WO2018060453, WO2018060462, WO2018060480, WO2018062361, WO2018062402, WO2018064190, WO2018064205, WO2018064255, WO2018064299, WO2018064436, WO2018064478, WO2018064594 WO2018064602, WO2018064603, WO2018064611, WO2018065501, WO2018065552, WO2018066585, WO2018066626, WO2018067198, WO2018067331, WO2018067331, WO2018067580, WO2018067582, WO2018067602, WO2018067618, WO2018067991, WO2018067992, WO2018067993, WO2018068028, WO2018068182, WO2018068201, WO2018068282, WO2018068283, WO2018068336, WO2018068354, WO2018068652, WO2018068691, WO2018068695, WO2018068758, WO2018069279, WO2018069467, WO2018069480, WO2018069500, WO2018069871, WO2018069927, WO2018070390, WO2018070479, WO2018070936, WO2018071500, WO2018071583, WO2018071624, WO2018071676, WO2018071701, WO2018071777, WO2018071792, WO2018071796, WO2018071871, WO2018071873, WO2018071910, WO2018071913, WO2018071919, WO2018071959, WO2018072025, WO2018072743, WO2018073363, WO2018073593, WO2018073648, WO2018073680, WO2018073721, WO2018074978, WO2018075304, WO2018075375, WO2018075564, WO2018075586, WO2018075591, WO2018075600, WO2018075693, WO2018075740, WO2018075794, WO2018075807, WO2018075813, WO2018075820, WO2018075960, WO2018075980, WO2018075989, WO2018076024, WO2018076828, WO2018077775, WO2018077893, WO2018077926, WO2018078442, WO2018078643, WO2018079314, WO2018079393, WO2018079740. WO2018080015, WO2018080812, WO2018080914, WO2018081074, WO2018081287, WO2018081329, WO2018081370, WO2018081375, WO2018081437, WO2018081448, WO2018081512, WO2018081648, WO2018081649, WO2018081754, WO2018081755, WO2018081832, WO2018081897, WO2018081898, WO2018082590, WO2018083071, WO2018083087, WO2018083248, WO2018083282, WO2018083538, WO2018083633, WO2018084236, WO2018084836, WO2018085252, WO2018085358, WO2018085359, WO2018085431, WO2018085441, WO2018085444, WO2018085468, WO2018085469, WO2018085533, WO2018085555, WO2018085599, WO2018085731, WO2018085815, WO2018086139, WO2018086585, WO2018086605, WO2018087143, WO2018087172, WO2018087276, WO2018087644, WO2018088850, WO2018088877, WO2018088878, WO2018089293, WO2018089305, WO2018089335, WO2018089393, WO2018089508, WO2018089532, WO2018089628, WO2018089807, WO2018089829, WO2018089890, WO2018090057, WO2018090950, WO2018091444, WO2018091606, WO2018091661, WO2018091720, WO2018091739, WO2018091740, WO2018092885, WO2018092907, WO2018093668, WO2018093821, WO2018093841, WO2018093866, WO2018094021, WO2018094143, WO2018094144, WO2018094282, WO2018094300, WO2018094414, WO2018094460, WO2018095428, WO2018095932, WO2018096467, WO2018097951, WO2018098035, WO2018098168, WO2018098258, WO2018098277, WO2018098306, WO2018098312, WO2018098348, WO2018098354, WO2018098356, WO2018098362, WO2018098363, WO2018098365, WO2018098370, WO2018099539, WO2018099614, WO2018099968, WO2018099972, WO2018099978, WO2018100534, WO2018100535, WO2018100558, WO2018100628, WO2018101448, WO2018101470, WO2018102294, WO2018102304, WO2018102536, WO2018102589, WO2018102594, WO2018102597, WO2018102612, WO2018102668, WO2018102682, WO2018102746, WO2018102752, WO2018102785, WO2018102786, WO2018102787, WO2018102795, WO2018103093, WO2018103501, WO2018103503, WO2018103884, WO2018104407, WO2018104444, WO2018104478, WO2018104483, WO2018104554, WO2018104556, WO2018104562, WO2018106529, WO2018106588, WO2018106644, WO2018106645, WO2018106732, WO2018106776, WO2018106781, WO2018106862, WO2018106864, WO2018107058, WO2018107069, WO2018107109, WO2018107116, WO2018107125, WO2018107134, WO2018108106, WO2018109170, WO2018109174, WO2018109213, WO2018109222, WO2018109770, WO2018110515, WO2018110555, WO2018111852, WO2018111890, WO2018112266, WO2018112334, WO2018112346, WO2018112407, WO2018112426, WO2018112549, WO2018113136, WO2018113258, WO2018113595, WO2018114578, WO2018114728, WO2018114748, WO2018114754, WO2018114798, WO2018114804, WO2018115017, WO2018115051, WO2018115231, WO2018115262, WO2018115319, WO2018115377, WO2018115466, WO2018115485, WO2018115885, WO2018115887, WO2018115960, WO2018116178, WO2018117044, WO2018117237, WO2018118713, WO2018118780, WO2018119001, WO2018119016, WO2018119114, WO2018119118, WO2018119166, WO2018119171, WO2018119196, WO2018119215, WO2018119246, WO2018119279, WO2018119314, WO2018119351, WO2018119380, WO2018119425, WO2018119474, WO2018119475, WO2018120842, WO2018120843, WO2018121473, WO2018121474, WO2018121475, WO2018121476, WO2018121578, WO2018121580, WO2018121679, WO2018122053, WO2018122204, WO2018123949, WO2018124851, WO2018126232, WO2018126233, WO2018126259, WO2018126317, WO2018126595, WO2018127175, WO2018127473, WO2018127519, WO2018127586, WO2018127608, WO2018127610, WO2018127709, WO2018127710, WO2018127711, WO2018127713, WO2018127787, WO2018127791, WO2018128485, WO2018128486, WO2018128691, WO2018128779, WO2018128939, WO2018128973, WO2018129007, WO2018129029, WO2018129090, WO2018129261, WO2018129329, WO2018129331, WO2018129400, WO2018129404, WO2018129425, WO2018129451, WO2018129474, WO2018129522, WO2018129524, WO2018129533, WO2018129553, WO2018129559, WO2018129714, WO2018130513, WO2018130657, WO2018131586, WO2018132597, WO2018132739, WO2018132768, WO2018132976, WO2018133634, WO2018133649, WO2018133837, WO2018133842, WO2018133873, WO2018133877, WO2018134234, WO2018134235, WO2018134279, WO2018134681, WO2018134691, WO2018134782, WO2018134784, WO2018134787, WO2018134817, WO2018135653, WO2018136152, WO2018136163, WO2018136412, WO2018136455, WO2018136553, WO2018136562, WO2018136570, WO2018136698, WO2018136823, WO2018136825, WO2018136891, WO2018136910, WO2018137293, WO2018137294, WO2018137295, WO2018137576, WO2018137598, WO2018137705, WO2018138032, WO2018138113, WO2018138297, WO2018138496, WO2018138521, WO2018138681, WO2018139404, WO2018139623, WO2018140026, WO2018140510, WO2018140525, WO2018140586, WO2018140660, WO2018140725, WO2018140733, WO2018140831, WO2018140845, WO2018140970, WO2018140973, WO2018141910, WO2018141959, WO2018141964, WO2018142322, WO2018142323, WO2018143454, WO2018143938, WO2018144097, WO2018144410, WO2018144425, WO2018144535, WO2018144637, WO2018144999, WO2018145075, WO2018145120, WO2018145206, WO2018145533, WO2018146074, WO2018146189, WO2018146199, WO2018146230, WO2018146253, WO201814317, WO2018146321, WO2018146549, WO2018146612, WO2018147245, WO2018148180, WO2018148223, WO201814224, WO2018148383, WO2018148445, WO2018148447, WO2018148454, WO2018148476, WO2018148486, WO2018148494, WO2018148566, WO2018148585, WO2018148595, WO2018148610, WO2018148660, WO2018149358, WO2018149938, WO2018149956, WO2018150029, WO2018150187, WO2018150224, WO2018150326, WO2018151816, WO2018151817, WO2018151820, WO2018151821, WO2018151836, WO2018151841, WO2018151868, WO2018152181, WO2018152516, WO2018152518, WO2018152530, WO2018152547, WO2018152634, WO2018152687, WO2018153320, WO2018153340, WO2018153366, WO2018153372, WO2018153865, WO2018154386, WO2018154391, WO2018154520, WO2018154580, WO2018156180, WO2018156250, WO2018156367, WO2018156494, WO2018156509, WO2018156634, WO2018156735, WO2018156740, WO2018156791, WO2018157147, WO2018157169, WO2018158335, WO2018158398, WO2018158632, WO2018158658, WO2018158727, WO2018158741, WO2018159582, WO2018159845, WO2018160536, WO2018160538, WO2018160539, WO2018160704, WO2018160731, WO2018160841, WO2018160909, WO2018160917, WO2018161017, WO2018161092, WO2018161340, WO2018161798, WO2018161872, WO2018162376, WO2018162430, WO2018162446, WO2018162724, WO2018162727, WO2018162749, WO2018162944, WO2018163185, WO2018164385, WO2018164441, WO2018164637, WO2018165062, WO2018165186, WO2018165228, WO2018165362, WO2018165475, WO2018165619, WO2018165895, WO2018166468, WO2018166589, WO2018167104, WO2018167151, WO2018167267, WO2018167322, WO2018168768, WO2018169785, WO2018169948, WO2018169953, WO2018169993, WO2018169999, WO2018170134, WO2018170145, WO2018170168, WO2018170188, WO2018170256, WO2018170338, WO2018170351, WO2018170359, WO2018170458, WO2018172078, WO2018172465, WO2018174408, WO2018174544, WO2018174629, WO2018174984, WO2018175179, WO2018175403, WO2018175476, WO2018175740, WO2018175752, WO2018175788, WO2018175790, WO2018175833, WO2018175917, WO2018175988, WO2018176089, WO2018176132, WO2018176505, WO2018176992, WO2018177220, WO2018177324, WO2018177393, WO2018177966, WO2018177967, WO2018178040, WO2018178046, WO2018178047, WO2018178055, WO2018178076, WO2018178077, WO2018178101, WO2018178122, WO2018178123, WO2018178307, WO2018178352, WO2018178354, WO2018178363, WO2018178364, WO2018178378, WO2018178950, WO2018179302, WO2018181064, WO2018181207, WO2018181656, WO2018182266, WO2018182284, WO2018182422, WO2018182529, WO2018183041, WO2018183182, WO2018183293, WO2018183294, WO2018183366, WO2018183485, WO2018183494, WO2018183608, WO2018184558, WO2018184593, WO2018184964, WO2018184965, WO2018184966, WO2018185043, WO2018185045, WO2018185050, WO2018185110, WO2018185232, WO2018185247, WO2018185284, WO2018185526, WO2018185618, WO2018185709, WO2018187158, WO2018187190, WO2018187191, WO2018187332, WO2018187453, WO2018187512, WO2018187518, WO2018187613, WO2018187682, WO2018187702, WO2018187706, WO2018187791, WO2018187799, WO2018188047, WO2018188612, WO2018189220, WO2018189379, WO2018189381, WO2018189382, WO2018189520, WO2018189611, WO2018190719, WO2018191074, WO2018191188, WO2018191313, WO2018191414, WO2018191422, WO2018191438, WO2018191502, WO2018191545, WO2018191548, WO2018191660, WO2018191707, WO2018191718, WO2018191723, WO2018192089, WO2018193063, WO2018193427, WO2018193457, WO2018194380, WO2018194381, WO2018194944, WO2018195034, WO2018195226, WO2018195283, WO2018195302, WO2018195388, WO2018195418, WO2018197492, WO2018197675, WO2018198091, WO2018199176, WO2018199318, WO2018199593, WO2018200422, WO2018200430, WO2018200496, WO2018200562, WO2018200585, WO2018200586, WO2018200812, WO2018200818, WO2018200884, WO2018200918, WO2018201014, WO2018201047, WO2018201051, WO2018201056, WO2018201087, WO2018201096, WO2018201099, WO2018202649, WO2018202794, WO2018203289, WO2018203517, WO2018203567, WO2018204156, WO2018204278, WO2018204303, WO2018204343, WO2018204363, WO2018204368, WO2018204374, WO2018204405, WO2018204427, WO2018204489, WO2018204520, WO2018204546, WO2018204677, WO2018204679, WO2018204757, WO2018204868, WO2018204871, WO2018204872, WO2018204876. WO2018204895, WO2018205343, WO2018205344, WO2018205917, WO2018205926, WO2018205985, WO2018206496, WO2018206565, WO2018206734, WO2018206790, WO2018208091, WO2018208121, WO2018208122, WO2018208231, WO2018208625, WO2018208670, WO2018208709, WO2018208856, WO2018208868, WO2018209175, WO2018209194. WO2018209223, WO2018209344, WO2018209346, WO2018209701, WO2018210230, WO2018210793, WO2018210795, WO2018210898, WO2018212136, WO2018212656, WO2018213064, WO2018213113, WO2018213204, WO2018213260, WO2018213297, WO2018213304, WO2018213316, WO2018213331, WO2018213335, WO2018213337, WO2018213430, WO2018213592, WO2018213612, WO2018213665, WO2018213679, WO2018213680, WO2018213848, WO2018215535, WO2018215571, WO2018215614, WO2018215768, WO2018215831, WO2018215835, WO2018215935, WO2018215936, WO2018215937, WO2018215938, WO2018215995, WO2018217058, WO2018217116, WO2018217227, WO2018217459, WO2018217638, WO2018217688, WO2018217799, WO2018217918, WO2018217940, WO2018217945, WO2018217947, WO2018217976, WO2018217988, WO2018217988, WO2018217989, WO2018218049, WO2018218056, WO2018218068, WO2018218083, WO2018218185, WO2018218215, WO2018218219, WO2018218222, WO2018218240, WO2018218352, WO2018218875, WO2018218876, WO2018218877, WO2018218878, WO2018218879, WO2018219299, WO2018219327, WO2018219901, WO2018219956, WO2018220080, WO2018220099, WO2018220100, WO2018220169, WO2018220216, WO2018220225, WO2018220234, WO2018220235, WO2018220236, WO2018220467, WO2018220546, WO2018220584. WO2018221521, WO2018221969, WO2018222019, WO2018222139, WO2018222433, WO2018222675, WO2018222685, WO2018222689, WO2018222711, WO2018222718, WO2018222722, WO2018222854, WO2018222901, WO2018222935, WO2018222949, WO2018222962, WO2018223002, WO2018223004, WO2018223051, WO2018223098, WO2018223101, WO2018223182, WO2018223243, WO2018223600, WO2018223601, WO2018223923, WO2018223958, WO2018224443, WO2018224550, WO2018224609, WO2018224682, WO2018224683, WO2018224685, WO2018224983, WO2018225041, WO2018225732, WO2018226081, WO2018226578, WO2018226580, WO2018226776, WO2018226795, WO2018226833, WO2018226958, WO2018226985, WO2018226992, WO2018227018, WO2018228406, WO2018228442, WO2018229193, WO2018229194, WO2018229195, WO2018229197, WO2018229222, WO2018229249, WO2018229530, WO2018229612, WO2018229706, WO2018229715, WO2018231254, WO2018231339, WO2018231827, WO2018232088, WO2018232144, WO2018232164, WO2018232188, WO2018232349, WO2018232355, WO2018232366, WO2018232372, WO2018232467, WO2018233333, WO2018233511, WO2018234319, WO2018234383, WO2018234575, WO2018234576, WO2018234793, WO2018235855, WO2018236728, WO2018236904, WO2018237006, WO2018237010, WO2018237037, WO2018237064, WO2018237148, WO2018237157, WO2018237173, WO2018237192, WO2018237287, WO2018237326, WO2018237335, WO2018237341, WO2018237357, WO2018237364, WO2019000105, WO2019001417, WO2019001559, WO2019002444, WO2019003074, WO2019003114, WO2019003162, WO2019003164, WO2019003165, WO2019004550, WO2019004831, WO2019004943, WO2019005208, WO2019005503, WO2019005634, WO2019005635, WO2019005636, WO2019005638, WO2019005756, WO2019005817, WO2019005847, WO2019005897, WO2019006007, WO2019006043, WO2019006072, WO2019006159, WO2019006162, WO2019006280, WO2019006472, WO2019006529, WO2019008377, WO2019008378, WO2019008379, WO2019009231, WO2019009346, WO2019009388, WO2019009684, WO2019009726, WO2019009727, WO2019009728, WO2019009879, WO2019010219, WO2019010224, WO2019010314, WO2019010486, WO2019010566, WO2019011167, WO2019011306, WO2019011852, WO2019011855, WO2019011918, WO2019012014, WO2019012015, WO2019012138, WO2019012141, WO2019012260, WO2019013392, WO2019013394, WO2019014091, WO2019014328, WO2019014360, WO2019014405, WO2019014456, WO2019014572, WO2019014586, WO2019014623, WO2019014768, WO2019015282, WO2019015677, WO2019015696, WO2019015936, WO2019016213, WO2019016237, WO2019016371, WO2019016381, WO2019016392, WO2019016402, WO2019016411, WO2019016784, WO2019017401, WO2019018310, WO2019018382, WO219018402, WO2019018426, WO2019018428, WO2019018538, WO2019018629, WO2019018640, WO2019018647, WO2019018730, WO2019018757, WO2019018770, WO2019018828, WO2019020745, WO2019020774, WO2019022187, WO2019022274, WO2019022281, WO2019023056, WO2019023097, WO2019023148, WO2019023247, WO2019023347, WO2019023396, WO2019023410, WO2019023460, WO2019023482, WO2019023504, WO2019023564, WO2019023661, WO2019023811, WO2019023812, WO2019024911, WO2019024933, WO2019025299, WO2019025484, WO2019025545, WO2019027754, WO2019027780, WO2019027903, WO2019027935, WO2019028027, WO2019028051, WO2019028125, WO2019028182, WO2019028190, WO2019028283, WO2019028292, WO2019028316, WO2019028367. WO2019028417, WO2019028427, WO2019028456, WO2019028530, WO2019028555, WO2019028980, WO2019029351, WO2019030202, WO2019030223, WO2019030240, WO2019030377, WO2019030757, WO2019031938, WO2019031939, WO2019031965, WO2019032661, WO2019032662, WO2019032663, WO2019032699, WO2019032898, WO2019032916. WO2019032927, WO2019032929, WO2019032945, WO2019033043, WO2019033050, WO2019033057, WO2019033087, WO2019033114, WO2019034177, WO2019034427, WO2019034752, WO2019034753, WO2019034764, WO2019034779, WO2019034895, WO2019035005, WO2019035630, WO2019035649, WO2019035938, WO2019035939, WO2019036605, WO2019036688, WO2019036724, WO2019036725, WO2019036855, WO2019037099, WO2019037711, WO2019038368, WO2019039483, WO2019040348, WO2019040390, WO2019040608, WO2019040612, WO2019040685, WO2019040727, WO2019040780, WO2019040791, WO2019040808, WO2019041024, WO2019042153, WO2019042226, WO2019042282, WO2019042285, WO2019043059, WO2019043065, WO2019045086, WO2019045452, WO2019045856, WO2019046225, WO2019046321, WO2019046338, WO2019046600, WO2019046652, WO2019046817, WO2019046856, WO2019046858, WO2019046859, WO2019047921, WO2019047932, WO2019048040, WO2019048489, WO2019050273, WO2019050275, WO2019050326, WO2019050362, WO2019050780, WO2019050935, WO2019051091, WO2019051102, WO2019051122, WO2019051127, WO2019051128, WO2019051132, WO2019051164, WO2019051308, WO2019051335, WO2019051397, WO2019051501, WO2019051586, WO2019052562, WO2019053420, WO2019053611, WO2019053612, WO2019053613, WO2019054460, WO219054819, WO2019055399, WO2019055537, WO2019055631, WO2019055677, WO2019055706, WO2019055776, WO2019055842, WO2019056023, WO2019056099, WO2019056106, WO2019056281, WO2019057099, WO2019057100, WO2019057102, WO2019057124, WO2019057180, WO2019057560, WO2019057567, WO2019057772, WO2019057780, WO2019057787, WO2019057792, WO2019057816, WO2019057933, WO2019057992, WO2019058345, WO2019059411, WO2019059771, WO2019060418, WO2019060542, WO2019060695, WO2019060707, WO2019060718, WO2019060750, WO2019061962, WO2019062518, WO2019062642, WO2019062755, WO2019062817, WO2019062831, WO2019062832, WO2019062871, WO2019062877, WO2019063018, WO2019063802, WO2019064073, WO2019065964, WO2019066435, WO2019066535, WO2019066536, WO2019066617, WO2019066620, WO2019067015, WO2019067249, WO2019067332, WO2019067491, WO2019067499, WO2019067805, WO2019067815, WO2019067951, WO2019067978, WO2019068633, WO2019068733, WO2019068842, WO2019068904, WO2019068907, WO2019069125, WO2019069229, WO2019070013, WO2019070161, WO2019070435, WO2019070541, WO2019070577, WO2019070680, WO2019070714, WO2019070726, WO2019070740, WO2019070834, WO2019071164, WO2019071205, WO2019072220, WO2019072274, WO2019072566, WO2019072869, WO2019072870, WO2019072977, WO2019073069, WO2019073080, WO2019074124, WO2019074333, WO2019074498, WO2019074973, WO2019075097, WO2019075136, WO2019075168, WO2019075188, WO2019075216, WO2019075220, WO2019075270, WO2019075300, WO2019075359, WO2019075378, WO2019075385, WO2019075392, WO2019075405, WO2019075413, WO2019075417, WO2019075472, WO2019075487, WO2019076277, WO2019076486, WO2019077092, WO2019077132, WO2019077951, WO2019078600, WO2019078697, WO2019078698, WO2019078699, WO2019079249, WO2019079337, WO2019079569, WO2019079671 WO2019079762, WO2019079772, WO2019079809, WO2019079914, WO2019080858, WO2019080872, WO2019080883, WO2019080889, WO2019080909, WO2019080941, WO2019081595, WO2019081692, WO2019081902, WO2019081983, WO2019082020, WO2019082208, WO2019083506, WO2019084024, WO2019084053, WO2019084055, WO2019084057, WO2019084060, WO2019084064, WO2019084067, WO2019084249, WO2019084288, WO2019084307, WO2019084319, WO2019084332, WO2019084438, WO2019084460, WO2019084552, WO2019084553, WO2019084692, WO2019085102, WO2019085238, WO2019085804. WO2019085902, WO2019086362, WO2019086497, WO2019086499, WO2019086500, WO2019086512, WO2019086573, WO2019086574, WO2019086580, WO2019086878, WO2019087087, WO2019087094, WO2019087115, WO2019087133, WO2019087994, WO2019088658, WO2019089395, WO2019089472, WO2019089473, WO2019089544, WO2019089592, WO2019089594, WO2019089610, WO2019089753, WO2019089755, WO2019089848, WO2019089855, WO2019089858, WO2019089870, WO2019089921, WO2019089969, WO2019089973, WO2019089982, WO2019090002, WO2019090003, WO2019090004, WO2019090069, WO2019090074, WO2019090076, WO2019090078, WO2019090081, WO2019090082, WO2019090085, WO2019090088, WO2019090090, WO2019090110, WO2019090134, WO2019090263, WO2019090329, WO2019090355, WO2019091384, WO2019092148, WO2019092181, WO2019092451, WO2019092452, WO2019092505, WO2019092507, WO2019092677, WO2019093342, WO2019093807, WO2019094265, WO2019094482, WO2019094578, WO2019094595, WO2019094626, WO2019094637, WO2019094700, WO2019094983, WO2019096115, WO2019096121, WO2019096136, WO2019096788, WO2019096874, WO2019096900, WO2019097244, WO2019097305, WO2019099374, WO2019099433, WO2019099440, WO2019099479, WO2019099483, WO2019099597, WO2019099744, WO2019099882, WO2019099993, WO2019100052, WO2019098212, WO2019098133, WO2019087155, WO2019085982, WO2019084427, WO2019076361, WO2019065979, WO2019056991, WO2019051541, WO2019040453, WO2019040230, WO2019036433, WO2019035241, WO2019034580, WO2019030260, WO2019027828, WO2019025983, WO2019025391, WO2019024979, WO2019013308, WO2019006330, WO2019004213, WO2018235964, WO2018235247, WO2018235024, WO2018227686, WO2018224441, WO2018217630, WO2018208553, WO2018200582, WO2018187057, WO2018183139, WO2018182421, WO2018182420, WO2018177369, WO2018175453, WO2018170096, WO2018158349, WO2018147960, WO2018147915, WO2018147432, WO2018145649, WO2018145648, WO2018140121, WO2018132635, WO2018131893, WO2018131718, WO2018129397, WO2018126369, WO2018111662, WO2018108759, WO2018107388, WO2018102473, WO2018099402, WO2018097724, WO2018089300, WO2018087349, WO2018086239, WO2018083535, WO2018083240, WO2018083238, WO2018083237, WO2018083235, WO2018079857, WO2018078186, WO2018078123, WO2018068766, WO2018067754, WO2018065389, WO2018064515, WO2018062495, WO2018057849, WO2018057051, WO2018053649, WO2018051348 WO2018044172, WO2018035330, WO2018032574, WO2018030405, WO2018021979, WO2018017863, WO2018014067, WO2018011555, WO2018011420, WO2018007456, WO2017222056, WO2017218750, WO2017218691, WO2017198811, WO2017191327, WO217189983, WO2017183028, WO2017177032, WO2017141604, WO2017141208, WO2017140831, WO2017136524, WO2017134134, WO2017133682, WO2017133222, WO2017132259, WO2017121867, WO2017121307, WO2017120614, WO2017113181, WO2017112621, WO2017105091, WO2017104778, WO2017102920, WO2017102830, WO2017097889, WO2017091905, WO2017082512, WO2017076805, WO2017076804, WO2017075229, WO2017075045, WO2017072716, WO2017072183, WO2017071513, WO2017055966, WO2017055542, WO2017046994, WO2017046212, WO2017046211, WO2017046208, WO2017046207, WO2017046205, WO2017046202, WO2017046201, and WO2017046198, the contents of each of which are herein incorporated by reference in their entirety.

In some embodiments, payloads may encode cancer and immunoinflammatory disease-associated antibodies (or fragments thereof) as described in Arenas-Ramirez et al. (Sci Transl Med, November 2016, Vol. 8(367), p 367ra166; the contents of which are herein incorporated by reference in their entirety). Such embodiments may include antibody NARA1 or fragments thereof.

In some embodiments, payloads may encode cancer and immunoinflammatory disease-associated antibodies (or fragments thereof) taught in International Publication Number WO2014028776 and US Patent Publication Number US20180201692, the contents of each of which are herein incorporated by reference in their entirety. Such embodiments may include antibody Trastuzumab or fragments thereof. In certain embodiments, the payload region encodes antibody Trastuzumab or fragments thereof selected from SEQ ID NO: 55-62, as described in WO2014028776. In certain embodiments, the payload region encodes antibody Trastuzumab or fragments thereof selected from SEQ ID NO: 1-24, as described in US20180201692.

In some embodiments, payloads may encode cancer and immunoinflammatory disease-associated antibodies (or fragments thereof) taught in International Publication Number WO2018089788, the contents of which are herein incorporated by reference in their entirety. Such embodiments may include antibody Tremelimumab or fragments thereof. In certain embodiments, the payload region encodes antibody Tremelimumab or fragments thereof selected from SEQ ID NO: 9-16, as described in WO2018089788.

In some embodiments, payloads may encode cancer and immunoinflammatory disease-associated antibodies (or fragments thereof) taught in International Publication Number WO2016201388, the contents of which are herein incorporated by reference in their entirety. Such embodiments may include antibody anti-CD33 or fragments thereof. In certain embodiments, the payload region encodes antibody anti-CD33 or fragments thereof selected from SEQ ID NO: 248-251, as described in WO2016201388.

In some embodiments, payloads may encode cancer and immunoinflammatory disease-associated antibodies (or fragments thereof) taught in US Patent Publication Number US20180333503, the contents of which are herein incorporated by reference in their entirety. Such embodiments may include antibody Pembrolizumab or fragments thereof. In certain embodiment, the payload region encodes antibody Pembrolizumab or fragments thereof selected from SEQ ID NO: 20.29 as described in US20180333503.

In some embodiments, payloads may encode cancer and immunoinflammatory disease-associated antibodies (or fragments thereof) taught in International Publication Number WO2018089780, the contents of which are herein incorporated by reference in their entirety. Such embodiments may include antibody Durvalumab (Imfinzi, MEDI-4736, MEDI-4736) or fragments thereof. In certain embodiments, the payload region encodes antibody Durvalumab (Imfinzi, MEDI-4736, and MEDI-4736) or fragments thereof selected from SEQ ID NO: 1-8 as described in WO2018089780.

In some embodiments, payloads may encode cancer and immunoinflammatory disease-associated antibodies (or fragments thereof) taught in International Publication Number WO2010129469 and WO201012949, the contents of each of which are herein incorporated by reference in their entirety. Such embodiments may include antibody Adalimumab or fragments thereof. In certain embodiments, the payload region encodes the antibody Adalimumab or fragments thereof selected from SEQ ID NO: 76-83 as described in WO2010129469. In certain embodiments, the payload region encodes the antibody Adalimumab or fragments thereof selected from SEQ ID NO: 1-37 as described in WO2010129469.

In some embodiments, payloads may encode cancer and immunoinflammatory disease-associated antibodies (or fragments thereof) taught in International Publication Number WO2014028776 and in US Patent Publication Number US20190137523, the contents of each of which are herein incorporated by reference in their entirety. Such embodiments may include antibody Bevacizumab or fragments thereof. In certain embodiments, the payload region encodes antibody Bevacizumab or fragments thereof selected from SEQ ID NO: 68-75, as described in WO2014028778. In certain embodiments, the payload region encodes antibody Bevacizumab or fragments thereof selected from SEQ ID NO: 2-5, as described in US20190137523.

In some embodiments, payloads may encode cancer and immunoinflammatory disease-associated antibodies (or fragments thereof) taught in International Publication Number US20180221480, the contents of which are herein incorporated by reference in their entirety. Such embodiments may include antibody natalizumab or fragments thereof. In certain embodiments, the payload region encodes antibody natalizumab or fragments thereof selected from SEQ ID NO: 1-14, as described in US20180221480.

In some embodiments, payloads may encode cancer and immunoinflammatory disease-associated antibodies (or fragments thereof) taught in US Patent Publication Number US2018005106, the contents of which are herein incorporated by reference in their entirety. Such embodiments may include antibody Vedolizumab (Entyvio) or fragments thereof. In certain embodiments, the payload region encodes antibody Vedolizumab (Entyvio) or fragments thereof selected from SEQ ID NO: 1-13 as described in US20180051086.

In some embodiments, payloads may encode cancer and immunoinflammatory disease-associated antibodies (or fragments thereof) taught in US Patent Publication Number US2019009243, the contents of which are herein incorporated by reference in their entirety. Such embodiments may include antibody Eculizumab or fragments thereof. In certain embodiments, the payload region encodes antibody Eculizumab or fragments thereof selected from SEQ ID NO: 1-3, as described in US20190092843.

In some embodiments, payloads may encode cancer and immunoinflammatory disease-associated antibodies (or fragments thereof) taught in International Publication Number WO2019079549 and US Patent Publication Number US20170253653, the contents of each of which are herein incorporated by reference in their entirety. Such embodiments may include antibody Avelumab or fragments thereof. In certain embodiments, the payload region encodes antibody Avelumab or fragments thereof selected from SEQ ID NO: 3-4, as described in WO2019079549. In certain embodiments, the payload region encodes antibody Avelumab or fragments thereof selected from SEQ ID NO: 1.35, as described in US20170253653.

In some embodiments, payloads may encode cancer and immunoinflammatory disease-associated antibodies (or fragments thereof) taught in International Publication Number WO2019079549, the contents of which are herein incorporated by reference in their entirety. Such embodiments may include antibody anti-CD47 antibody Hu5f9-G4 Antibody or fragments thereof. In certain embodiments, the payload region encodes antibody anti-CD47 antibody Hu5f9-G4 Antibody or fragments thereof selected from SEQ ID NO: 1-2, as described in WO2019079549.

In some embodiments, payloads may encode cancer and immunoinflammatory disease-associated antibodies (or fragments thereof) taught in US Patent Publication Number US20190135920, the contents of which are herein incorporated by reference in their entirety. Such embodiments may include antibody Nivolumab or fragments thereof. In certain embodiments, the payload region encodes antibody Nivolumab or fragments thereof selected from SEQ ID NO: 1-36, as described in US20190135920.

In some embodiments, payloads may encode cancer and immunoinflammatory disease-associated antibodies (or fragments thereof) taught in International Publication Number WO2018140121 and WO2018147927, the contents of each of which are herein incorporated by reference in their entirety. Such embodiments may include antibody Golimumab or fragments thereof. In certain embodiments, the payload region encodes antibody Golimumab or fragments thereof selected from SEQ ID NO: 36-37, as described in WO2018140121 and WO2018147927.

In some embodiments, payloads may encode cancer and immunoinflammatory disease-associated antibodies (or fragments thereof) taught in US Patent Publication Number US20140212413, the contents of which are herein incorporated by reference in their entirety. Such embodiments may include antibody Infliximab (Remicade) or fragments thereof. In certain embodiments, the payload region encodes antibody Infliximab (Remicade) or fragments thereof selected from SEQ ID NO: 2-5, as described in US20140212413.

In some embodiments, payloads may encode cancer and immunoinflammatory disease-associated antibodies (or fragments thereof) taught in International Publication Number WO2019020606, the contents of which are herein incorporated by reference in their entirety. Such embodiments may include antibody Rituximab or fragments thereof. In certain embodiments, the payload region encodes antibody Rituximab or fragments thereof selected from SEQ ID NO: 1-20, as described in WO2019020606.

In some embodiments, payloads may encode cancer and immunoinflammatory disease-associated antibodies (or fragments thereof) taught in US Patent Publication Number US20190117769, the contents of which are herein incorporated by reference in their entirety. Such embodiments may include antibody Pertuzumab or fragments thereof. In certain embodiments, the payload region encodes antibody Pertuzumab or fragments thereof selected from SEQ ID NO: 11-12, 15-16, as described in US20190117769.

In some embodiments, payloads may encode cancer and immunoinflammatory disease-associated antibodies (or fragments thereof) taught in US Patent Publication Number US20190117769, the contents of which are herein incorporated by reference in their entirety. Such embodiments may include antibody Trastuzmab or fragments thereof. In certain embodiments, the payload region encodes antibody Trastuzmab or fragments thereof selected from SEQ ID NO: 13-14, as described in US20190117769.

In some embodiments, payloads may encode cancer and immunoinflammatory disease-associated antibodies (or fragments thereof) taught in US Patent Publication Number US20190016807, the contents of which are herein incorporated by reference in their entirety. Such embodiments may include antibody Atezolizumab or fragments thereof. In certain embodiments, the payload region encodes antibody Atezolizumab or fragments thereof selected from SEQ ID NO: 1-40 as described in US20190016807.

In some embodiments, payloads may encode cancer and immunoinflammatory disease-associated antibodies (or fragments thereof) taught in International Publication Number WO2013055922, the contents of which are herein incorporated by reference in their entirety. Such embodiments may include antibody Pepinemab or fragments thereof. In certain embodiments, the payload region encodes antibody Pepinemab or fragments thereof selected from SEQ ID NO: 9, 10, 17, 18 as described in WO2013055922.

In some embodiments, payloads may encode cancer and immunoinflammatory disease-associated antibodies (or fragments thereof) taught in International Publication Number WO2014093396, the contents of which are herein incorporated by reference in their entirety. Such embodiments may include anti blood dendritic cell antigen 2 (BDCA2) antibody or fragments thereof. Such embodiments may include antibody BIIB059 or fragments thereof.

In some embodiments, the payload region of the viral particle comprises one or more nucleic acid sequences encoding one or more of the cancer, inflammation and immune system payload antibody polypeptides listed in Table 9 of U.S. provisional patent application 62/844,433 (CII1-CII13310; SEQ ID NO: 6357-19665), the contents of which are herein incorporated by reference in their entirety.

In some embodiments, the payload region of the viral particle comprises one or more nucleic acid sequences encoding Priliximab, a fragment or variant thereof. As a non-limiting example, the payload region of the viral particle comprises one or more nucleic acid sequences encoding Priliximab may be used to treat, prevent and/or reduce the effects of multiple sclerosis. As another non-limiting example, the payload region of the viral particle comprises one or more nucleic acid sequences encoding Priliximab, a fragment or variant thereof may be used to treat, prevent and/or reduce the effects of Crohn's Disease.

In some embodiments, the payload region of the viral particle comprises one or more nucleic acid sequences encoding Rovelizumab, a fragment or variant thereof. As a non-limiting example, the payload region of the viral particle comprises one or more nucleic acid sequences encoding Rovelizumab, a fragment or variant thereof may be used to treat, prevent and/or reduce the effects of multiple sclerosis.

In some embodiments, the payload region of the viral particle comprises one or more nucleic acid sequences encoding Nerelimomab, a fragment or variant thereof. As a non-limiting example, the payload region of the viral particle comprises one or more nucleic acid sequences encoding Nerelimomab, a fragment or variant thereof may be used as an immunosuppressant.

In some embodiments, the payload region of the viral particle comprises one or more nucleic acid sequences encoding BAYX1351, a fragment or variant thereof. As a non-limiting example, the payload region of the viral particle comprises one or more nucleic acid sequences encoding BAYX1351, a fragment or variant thereof may be used as an immunosuppressant.

In some embodiments, the payload region of the viral particle comprises one or more nucleic acid sequences encoding Clenoliximab (also known as CE9γ4PE, IDEC-151 and PRIMATIZED®), a fragment or variant thereof. As a non-limiting example, the payload region of the viral particle comprises one or more nucleic acid sequences encoding Clenoliximab (also known as CE9γ4PE, IDEC-151 and PRIMATIZED®), a fragment or variant thereof may be used to treat, prevent or reduce the effects of rheumatoid arthritis and/or asthma. As a non-limiting example, the payload region of the viral particle comprises one or more nucleic acid sequences encoding the heavy chain of Clenoliximab (also known as CE9γ4PE, IDEC-151 and PRIMATIZED®), a fragment or variant thereof may be used to treat, prevent or reduce the effects of rheumatoid arthritis and/or asthma. As a non-limiting example, the payload region of the viral particle comprises one or more nucleic acid sequences encoding the light chain of Clenoliximab (also known as CE9γ4PE, IDEC-151 and PRIMATIZED®), a fragment or variant thereof may be used to treat, prevent or reduce the effects of rheumatoid arthritis and/or asthma. As a non-limiting example, the payload region of the viral particle comprises one or more nucleic acid sequences encoding the heavy chain of Clenoliximab (also known as CE9γ4PE, IDEC-151 and PRIMATIZED®) as described in U.S. Pat. No. 6,136,310 as SEQ ID NO: 11 (the contents of which are herein incorporated by reference in its entirety), a fragment or variant thereof may be used to treat, prevent or reduce the effects of rheumatoid arthritis and/or asthma. As a non-limiting example, the payload region of the viral particle comprises one or more nucleic acid sequences encoding the light chain of Clenoliximab (also known as CE9γ4PE, IDEC-151 and PRIMATIZED®) as described in U.S. Pat. No. 6,136,310 as SEQ ID NO: 5 (the contents of which are herein incorporated by reference in its entirety), a fragment or variant thereof may be used to treat, prevent or reduce the effects of rheumatoid arthritis and/or asthma.

In some embodiments, the payload region of the viral particle comprises one or more nucleic acid sequences encoding Maslimomab, a fragment or variant thereof. As a non-limiting example, the payload region of the viral particle comprises one or more nucleic acid sequences encoding Maslimomab, a fragment or variant thereof may be used as an immunosuppressant.

In some embodiments, the payload region of the viral particle comprises one or more nucleic acid sequences encoding Atorolimumab (also known as P3x22914G4), a fragment or variant thereof. As a non-limiting example, the payload region of the viral particle comprises one or more nucleic acid sequences encoding Atorolimumab (also known as P3x22914G4), a fragment or variant thereof may be used as an immunosuppressant.

In some embodiments, the payload region of the viral particle comprises one or more nucleic acid sequences encoding Vapaliximab (also known as 2D10), a fragment or variant thereof. As a non-limiting example, the payload region of the viral particle comprises one or more nucleic acid sequences encoding Vapaliximab (also known as 2D10), a fragment or variant thereof may be used as an immunosuppressant.

In some embodiments, the payload region of the viral particle comprises one or more nucleic acid sequences encoding Ziralimumab (also known as ABX-RB2, cem2.6), a fragment or variant thereof. As a non-limiting example, the payload region of the viral particle comprises one or more nucleic acid sequences encoding Ziralimumab (also known as ABX-RB2, cem2.6), a fragment or variant thereof may be used to treat, prevent and/or reduce the effects of cancer, inflammation and/or immune system disorders.

In some embodiments, the payload region of the viral particle comprises one or more nucleic acid sequences encoding Zolimomab aritox (also known as H65-ricin A chain immunotoxin and H65-RTA), a fragment or variant thereof. As a non-limiting example, the payload region of the viral particle comprises one or more nucleic acid sequences encoding Zolimomab aritox (also known as H65-ricin A chain immunotoxin and H65-RTA), a fragment or variant thereof may be used to treat, prevent or reduce the effects of systemic lupus erythematosus, graft-versus-host disease and/or cutaneous T cell lymphoma.

In some embodiments, the payload region of the viral particle comprises one or more nucleic acid sequences encoding Zanolimumab (also known as HuMax-CD4), a fragment or variant thereof. As a non-limiting example, the payload region of the viral particle comprises one or more nucleic acid sequences encoding Zanolmumab (also known as HuMax-CD4), a fragment or variant thereof may be used to treat, prevent or reduce the effects of rheumatoid arthritis, psoriasis and/or T-cell lymphoma.

In some embodiments, the payload region of the viral particle comprises one or more nucleic acid sequences encoding Bertilimumab (also known as CAT-213), a fragment or variant thereof. As a non-limiting example, the payload region of the viral particle comprises one or more nucleic acid sequences encoding Bertilimumab (also known as CAT-213), a fragment or variant thereof may be used to treat, prevent or reduce the effects of allergies.

In some embodiments, the payload region of the viral particle comprises one or more nucleic acid sequences encoding Pascolizumab (also known as SB-240683), a fragment or variant thereof. As a non-limiting example, the payload region of the viral particle comprises one or more nucleic acid sequences encoding Pascolizumab (also known as SB-240683), a fragment or variant thereof may be used to treat, prevent or reduce the effects of allergies.

In some embodiments, the payload region of the viral particle comprises one or more nucleic acid sequences encoding Odulimomab (also known as afolimomab, anti-LFA1 and ANTILFA), a fragment or variant thereof. As a non-limiting example, the payload region of the viral particle comprises one or more nucleic acid sequences encoding Odulimomab (also known as afolimomab, anti-LFA1 and ANTILFA), a fragment or variant thereof may be used to treat, prevent or reduce the effects of allograft rejection.

In some embodiments, the payload region of the viral particle comprises one or more nucleic acid sequences encoding Enlimomab pegol, a fragment or variant thereof. As a non-limiting example, the payload region of the viral particle comprises one or more nucleic acid sequences encoding Enlimomab pegol, a fragment or variant thereof may be used to treat, prevent or reduce the effects of renal transplant rejection.

In some embodiments, the payload region of the viral particle comprises a nucleic acid sequence encoding an antibody or a fragment thereof as described in United States Publication Nos. US20130122003, US20150056211, US20160069 US20150056211, US20160069894 or U.S. Pat. No. 7,524,496. In a non-limiting example, the antibody targets IL-6. In another non-limiting example, the antibody targets EGF.

### Antibodies for the Treatment of Blood and Blood Vessel Disease

In some embodiments, the payload region of the AAV particle comprises one or more nucleic acid sequences encoding blood and blood vessel disease-associated antibodies, variants or fragments thereof.

In some embodiments, the payload region of the AAV particle comprises one or more nucleic acid sequences encoding one or more of the payload antibody polypeptides listed in Table 5, or variants or fragments thereof.

In some embodiments, the payload region of the AAV particle comprises one or more nucleic acid sequences listed in Table 5, or variants or fragments thereof.

In some embodiments, the payload region of the AAV particle comprises a nucleic acid sequence encoding a payload antibody with at least 50% identity to one or more payload antibody polypeptides listed in Table 5. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%. 81%, 82%, 83%, 84%, 85%, 86%, 87%, 88%, 89%, 90%, 91%, 92%, 93%, 94%, 95%,96%,97%, 98%, 99%, or 100% identity to one or more of the payload antibody polypeptides listed in Table 5, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%. 90%, 91%, 92%, 93%, 94%, 95%, 96%, 97%, 98%, 99%, or 100% identity to one or more of the payload antibody polypeptides listed in Table 5, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 5, or variants or fragments thereof.

In some embodiments, the heavy chain of the encoded antibody polypeptide may have 50%,51%,52%, 53%,54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%,78%, 79%, 80%, 81%, 82%, 83%, 84%, 85%, 86%, 87%,88%, 89%, 90%, 91%, 92%, 93%, 94%, 95%, 96%, 97%, 98%, 99%, or 100% identity to one or more of the payload heavy chain antibody polypeptides listed in Table 5, or variants or fragments thereof.

In some embodiments, the light chain of the encoded antibody polypeptide may have 50%, 51%, 52%,53%, 54%, 55%, 56%, 57%, 58%, 59%, 60%, 61%, 62%, 63%, 64%, 65%, 66%,67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 5, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%,81%,82%, 83%,84%,85%, 86%, 87%, 88%, 89%, 90%,91%, 92%, 93%,94%,95%, 96%,97%,98%, 99%, or 100% identity to the CDRs of one or more of the payload antibody polypeptides listed in Table 5, or variants or fragments thereof.

In some embodiments, the payload antibody has 90% identity to one or more of the antibody polypeptides listed in Table 5, or variants or fragments thereof.

In some embodiments, the payload antibody has 91% identity to one or more of the antibody polypeptides listed in Table 5, or variants or fragments thereof.

In some embodiments, the payload antibody has 92% identity to one or more of the antibody polypeptides listed in Table 5, or variants or fragments thereof.

In some embodiments, the payload antibody has 93% identity to one or more of the antibody polypeptides listed in Table 5, or variants or fragments thereof.

In some embodiments, the payload antibody has 94% identity to one or more of the antibody polypeptides listed in Table 5, or variants or fragments thereof.

In some embodiments, the payload antibody has 95% identity to one or more of the antibody polypeptides listed in Table 5, or variants or fragments thereof.

In some embodiments, the payload antibody has 96% identity to one or more of the antibody polypeptides listed in Table 5, or variants or fragments thereof.

In some embodiments, the payload antibody has 97% identity to one or more of the antibody polypeptides listed in Table 5, or variants or fragments thereof.

In some embodiments, the payload antibody has 98% identity to one or more of the antibody polypeptides listed in Table 5, or variants or fragments thereof.

In some embodiments, the payload antibody has 99% identity to one or more of the antibody polypeptides listed in Table 5, or variants or fragments thereof.

In some embodiments, the payload antibody has 100% identity to one or more of the antibody polypeptides listed in Table 5, or variants or fragments thereof.

In some embodiments, the payload region of the AAV particle comprises a nucleic acid sequence with at least 50% identity to one or more nucleic acid sequences listed in Table 5, or variants or fragments thereof. The payload nucleic acid sequence may have 50%, 51%, 52%, 53%, 54%, 55%, 56%, 57%, 58%, 59%, 60%, 61%, 62%, 63%, 64%, 65%,66%,67%,68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 5, or variants or fragments thereof.

In some embodiments, the payload nucleic acid sequence has 90% identity to one or more of the nucleic acid sequences listed in Table 5, or variants or fragments thereof.

In some embodiments, the payload nucleic acid sequence has 91% identity to one or more of the nucleic acid sequences listed in Table 5, or variants or fragments thereof.

In some embodiments, the payload nucleic acid sequence has 92% identity to one or more of the nucleic acid sequences listed in Table 5, or variants or fragments thereof.

In some embodiments, the payload nucleic acid sequence has 93% identity to one or more of the nucleic acid sequences listed in Table 5, or variants or fragments thereof.

In some embodiments, the payload nucleic acid sequence has 94% identity to one or more of the nucleic acid sequences listed in Table 5, or variants or fragments thereof.

In some embodiments, the payload nucleic acid sequence has 95% identity to one or more of the nucleic acid sequences listed in Table 5, or variants or fragments thereof.

In some embodiments, the payload nucleic acid sequence has 96% identity to one or more of the nucleic acid sequences listed in Table 5, or variants or fragments thereof.

In some embodiments, the payload nucleic acid sequence has 97% identity to one or more of the nucleic acid sequences listed in Table 5, or variants or fragments thereof.

In some embodiments, the payload nucleic acid sequence has 98% identity to one or more of the nucleic acid sequences listed in Table 5, or variants or fragments thereof.

In some embodiments, the payload nucleic acid sequence has 99% identity to one or more of the nucleic acid sequences listed in Table 5, or variants or fragments thereof.

In some embodiments, the payload nucleic acid sequence has 100% identity to one or more of the nucleic acid sequences listed in Table 5, or variants or fragments thereof.

In some embodiments, the payload antibody may be variants of any of the antibody polypeptides listed in Table 5, that exclude one or more amino acids designated as “X” or “x” in the described polypeptide sequence, wherein X may represent any amino acid. In some embodiments, the payload nucleic acid sequence may be variants of any of the nucleic acid sequences listed in Table 5, that exclude one or more nucleic acids designated as “n” or “N” in the described nucleic acid sequence, wherein n may represent any nucleic acid.

TABLE 5
Blood and blood vessel disease antibodies

| | | Type | | SEQ |
|---|---|---|---|---|
| Ab ID | Component | (PRT/DNA) | Reference | ID NO. |
| BLD1 | CDR | PRT | WO2018054813; SEQ ID NO: 2 | 7347 |
| BLD2 | CDR | PRT | WO2018054813; SEQ ID NO: 9 | 7348 |
| BLD3 | CDR | PRT | WO2018054813; SEQ ID NO: 4 | 7349 |
| BLD4 | CDR | PRT | WO2018054813; SEQ ID NO: 12 | 7350 |
| BLD5 | CDR | PRT | WO2018054813; SEQ ID NO: 15 | 7351 |
| BLD6 | CDR | PRT | WO2018054813; SEQ ID NO: 5 | 7352 |
| BLD7 | CDR | PRT | WO2018054813; SEQ ID NO: 16 | 7353 |
| BLD8 | CDR | PRT | WO2018054813; SEQ ID NO: 13 | 7354 |
| BLD9 | CDR | PRT | WO2018054813; SEQ ID NO: 1 | 7355 |
| BLD10 | CDR | PRT | WO2018054813; SEQ ID NO: 8 | 7356 |
| BLD11 | CDR | PRT | WO2018054813; SEQ ID NO: 6 | 7357 |
| BLD12 | CDR | PRT | WO2018054813; SEQ ID NO: 14 | 7358 |
| BLD13 | CDR | PRT | WO2018054813; SEQ ID NO: 3 | 7359 |
| BLD14 | CDR | PRT | WO2018054813; SEQ ID NO: 10 | 7360 |
| BLD15 | CDR | PRT | WO2018054813; SEQ ID NO: 11 | 7361 |
| BLD16 | CDR | PRT | U.S. Pat. No. 8,080,243; SEQ ID NO: 46 | 7362 |
| BLD17 | CDR | PRT | U.S. Pat. No. 8,080,243; SEQ ID NO: 39 | 7363 |
| BLD18 | CDR | PRT | U.S. Pat. No. 8,080,243; SEQ ID NO: 49 | 7364 |
| BLD19 | CDR | PRT | U.S. Pat. No. 8,080,243; SEQ ID NO: 37 | 7365 |
| BLD20 | CDR | PRT | U.S. Pat. No. 8,080,243; SEQ ID NO: 44 | 7366 |
| BLD21 | CDR | PRT | U.S. Pat. No. 8,080,243; SEQ ID NO: 9 | 7367 |
| BLD22 | CDR | PRT | U.S. Pat. No. 8,080,243; SEQ ID NO: 52 | 7368 |
| BLD23 | CDR | PRT | U.S. Pat. No. 8,080,243; SEQ ID NO: 45 | 7369 |
| BLD24 | CDR | PRT | U.S. Pat. No. 8,080,243; SEQ ID NO: 10 | 7370 |
| BLD25 | CDR | PRT | U.S. Pat. No. 8,080,243; SEQ ID NO: 48 | 7371 |
| BLD26 | CDR | PRT | U.S. Pat. No. 8,080,243; SEQ ID NO: 58 | 7372 |
| BLD27 | CDR | PRT | U.S. Pat. No. 8,080,243; SEQ ID NO: 47 | 7373 |
| BLD28 | CDR | PRT | U.S. Pat. No. 8,080,243; SEQ ID NO: 36 | 7374 |
| BLD29 | CDR | PRT | U.S. Pat. No. 8,080,243; SEQ ID NO: 56 | 7375 |
| BLD30 | CDR | PRT | U.S. Pat. No. 8,080,243; SEQ ID NO: 41 | 7376 |
| BLD31 | CDR | PRT | U.S. Pat. No. 8,080,243; SEQ ID NO: 57 | 7377 |
| BLD32 | CDR | PRT | U.S. Pat. No. 8,080,243; SEQ ID NO: 59 | 7378 |
| BLD33 | CDR | PRT | U.S. Pat. No. 8,080,243; SEQ ID NO: 34 | 7379 |
| BLD34 | CDR | PRT | U.S. Pat. No. 8,080,243; SEQ ID NO: 30 | 7380 |
| BLD35 | CDR | PRT | U.S. Pat. No. 8,080,243; SEQ ID NO: 51 | 7381 |
| BLD36 | CDR | PRT | U.S. Pat. No. 8,080,243; SEQ ID NO: 38 | 7382 |
| BLD37 | CDR | PRT | U.S. Pat. No. 8,080,243; SEQ ID NO: 43 | 7383 |
| BLD38 | CDR | PRT | U.S. Pat. No. 8,080,243; SEQ ID NO: 50 | 7384 |
| BLD39 | CDR | PRT | U.S. Pat. No. 8,080,243; SEQ ID NO: 29 | 7385 |
| BLD40 | CDR | PRT | U.S. Pat. No. 8,080,243; SEQ ID NO: 35 | 7386 |
| BLD41 | CDR | PRT | U.S. Pat. No. 8,080,243; SEQ ID NO: 13 | 7387 |
| BLD42 | CDR | PRT | U.S. Pat. No. 8,080,243; SEQ ID NO: 31 | 7388 |
| BLD43 | CDR | PRT | U.S. Pat. No. 8,080,243; SEQ ID NO: 55 | 7389 |
| BLD44 | CDR | PRT | U.S. Pat. No. 8,080,243; SEQ ID NO: 11 | 7390 |
| BLD45 | CDR | PRT | U.S. Pat. No. 8,080,243; SEQ ID NO: 32 | 7391 |
| BLD46 | CDR | PRT | U.S. Pat. No. 8,080,243; SEQ ID NO: 61 | 7392 |
| BLD47 | CDR | PRT | U.S. Pat. No. 8,080,243; SEQ ID NO: 42 | 7393 |
| BLD48 | CDR | PRT | U.S. Pat. No. 8,080,243; SEQ ID NO: 8 | 7394 |
| BLD49 | CDR | PRT | U.S. Pat. No. 8,080,243; SEQ ID NO: 40 | 7395 |
| BLD50 | CDR | PRT | U.S. Pat. No. 8,080,243; SEQ ID NO: 33 | 7396 |
| BLD51 | Fab | PRT | WO2017180936; SEQ ID NO: 2 | 7397 |
| BLD52 | Fab | PRT | WO2017180936; SEQ ID NO: 33 | 7398 |
| BLD53 | HC | DNA | WO2018054813; SEQ ID NO: 32 | 7399 |
| BLD54 | HC | DNA | WO2018054813; SEQ ID NO: 34 | 7400 |
| BLD55 | HC | DNA | U.S. Pat. No. 8,080,243; SEQ ID NO: 26 | 7401 |
| BLD56 | HC | PRT | WO2018093766; SEQ ID NO: 26 | 7402 |
| BLD57 | HC | PRT | WO2018093766; SEQ ID NO: 30 | 7403 |
| BLD58 | HC | PRT | WO2018093766; SEQ ID NO: 28 | 7404 |
| BLD59 | HC | PRT | WO2018054813; SEQ ID NO: 30 | 7405 |
| BLD60 | HC | PRT | WO2018054813; SEQ ID NO: 28 | 7406 |
| BLD61 | HC | PRT | WO2018054813; SEQ ID NO: 36 | 7407 |
| BLD62 | HC | PRT | WO2018054813; SEQ ID NO: 38 | 7408 |
| BLD63 | HC | PRT | WO2018134184; SEQ ID NO: 1 | 7409 |
| BLD64 | HC | PRT | U.S. Pat. No. 8,080,243; SEQ ID NO: 15 | 7410 |
| BLD65 | HC | PRT | WO2017180936; SEQ ID NO: 1 | 7411 |
| BLD66 | LC | DNA | WO2018054813; SEQ ID NO: 33 | 7412 |
| BLD67 | LC | DNA | WO2018054813; SEQ ID NO: 31 | 7413 |
| BLD68 | LC | DNA | U.S. Pat. No. 8,080,243; SEQ ID NO: 25 | 7414 |
| BLD69 | LC | PRT | WO2018093766; SEQ ID NO: 29 | 7415 |
| BLD70 | LC | PRT | WO2018093766; SEQ ID NO: 18 | 7416 |
| BLD71 | LC | PRT | WO2018093766; SEQ ID NO: 27 | 7417 |
| BLD72 | LC | PRT | WO2018093766; SEQ ID NO: 16 | 7418 |
| BLD73 | LC | PRT | WO2018093766; SEQ ID NO: 17 | 7419 |
| BLD74 | LC | PRT | WO2018054813; SEQ ID NO: 27 | 7420 |
| BLD75 | LC | PRT | WO2018054813; SEQ ID NO: 35 | 7421 |
| BLD76 | LC | PRT | WO2018054813; SEQ ID NO: 19 | 7422 |
| BLD77 | LC | PRT | WO2018054813; SEQ ID NO: 29 | 7423 |
| BLD78 | LC | PRT | WO2018054813; SEQ ID NO: 37 | 7424 |
| BLD79 | LC | PRT | WO2018134184; SEQ ID NO: 2 | 7425 |
| BLD80 | LC | PRT | U.S. Pat. No. 8,080,243; SEQ ID NO: 14 | 7426 |
| BLD81 | scFv | PRT | WO2018093766; SEQ ID NO: 113 | 7427 |
| BLD82 | scFv | PRT | WO2018093766; SEQ ID NO: 114 | 7428 |
| BLD83 | scFv | PRT | WO2018093766; SEQ ID NO: 115 | 7429 |
| BLD84 | scFv | PRT | WO2018093766; SEQ ID NO: 116 | 7430 |
| BLD85 | scFv | PRT | WO2018093766; SEQ ID NO: 117 | 7431 |
| BLD86 | scFv | PRT | WO2018093766; SEQ ID NO: 118 | 7432 |
| BLD87 | scFv | PRT | WO2018093766; SEQ ID NO: 120 | 7433 |
| BLD88 | scFv | PRT | WO2018093766; SEQ ID NO: 121 | 7434 |
| BLD89 | scFv | PRT | WO2018093766; SEQ ID NO: 122 | 7435 |
| BLD90 | scFv | PRT | WO2018093766; SEQ ID NO: 123 | 7436 |
| BLD91 | scFv | PRT | WO2018093766; SEQ ID NO: 128 | 7437 |
| BLD92 | scFv | PRT | WO2018093766; SEQ ID NO: 129 | 7438 |
| BLD93 | scFv | PRT | WO2018093766; SEQ ID NO: 119 | 7439 |
| BLD94 | scFv | PRT | WO2018093766; SEQ ID NO: 125 | 7440 |
| BLD95 | scFv | PRT | WO2018093766; SEQ ID NO: 126 | 7441 |
| BLD96 | scFv | PRT | WO2018093766; SEQ ID NO: 127 | 7442 |
| BLD97 | scFv | PRT | WO2018093766; SEQ ID NO: 130 | 7443 |
| BLD98 | scFv | PRT | WO2018093766; SEQ ID NO: 131 | 7444 |
| BLD99 | scFv | PRT | WO2018093766; SEQ ID NO: 132 | 7445 |
| BLD100 | scFv | PRT | WO2018093766; SEQ ID NO: 133 | 7446 |
| BLD101 | scFv-Fc | PRT | WO2018093766; SEQ ID NO: 14 | 7447 |
| BLD102 | scFv-Fc | PRT | WO2018093766; SEQ ID NO: 10 | 7448 |
| BLD103 | scFv-Fc | PRT | WO2018093766; SEQ ID NO: 13 | 7449 |
| BLD104 | scFv-Fc | PRT | WO2018093766; SEQ ID NO: 9 | 7450 |
| BLD105 | scFv-Fc | PRT | WO2018093766; SEQ ID NO: 12 | 7451 |
| BLD106 | scFv-Fc | PRT | WO2018093766; SEQ ID NO: 11 | 7452 |
| BLD107 | scFv-Fc | PRT | WO2018093766; SEQ ID NO: 7 | 7453 |
| BLD108 | scFv-Fc | PRT | WO2018093766; SEQ ID NO: 8 | 7454 |
| BLD109 | scFv-Fc | PRT | WO2018093766; SEQ ID NO: 6 | 7455 |
| BLD110 | VH | PRT | WO2018093766; SEQ ID NO: 39 | 7456 |
| BLD111 | VH | PRT | WO2018093766; SEQ ID NO: 59 | 7457 |
| BLD112 | VH | PRT | WO2018093766; SEQ ID NO: 33 | 7458 |
| BLD113 | VH | PRT | WO2018093766; SEQ ID NO: 47 | 7459 |
| BLD114 | VH | PRT | WO2018093766; SEQ ID NO: 37 | 7460 |
| BLD115 | VH | PRT | WO2018093766; SEQ ID NO: 45 | 7461 |
| BLD116 | VH | PRT | WO2018093766; SEQ ID NO: 25 | 7462 |
| BLD117 | VH | PRT | WO2018093766; SEQ ID NO: 41 | 7463 |
| BLD118 | VH | PRT | WO2018093766; SEQ ID NO: 23 | 7464 |
| BLD119 | VH | PRT | WO2018093766; SEQ ID NO: 22 | 7465 |
| BLD120 | VH | PRT | WO2018093766; SEQ ID NO: 24 | 7466 |
| BLD121 | VH | PRT | WO2018093766; SEQ ID NO: 19 | 7467 |
| BLD122 | VH | PRT | WO2018093766; SEQ ID NO: 20 | 7468 |
| BLD123 | VH | PRT | WO2018093766; SEQ ID NO: 21 | 7469 |
| BLD124 | VH | PRT | WO2018093766; SEQ ID NO: 35 | 7470 |
| BLD125 | VH | PRT | WO2018093766; SEQ ID NO: 43 | 7471 |
| BLD126 | VH | PRT | WO2018093766; SEQ ID NO: 67 | 7472 |
| BLD127 | VH | PRT | WO2018093766; SEQ ID NO: 51 | 7473 |
| BLD128 | VH | PRT | WO2018054813; SEQ ID NO: 20 | 7474 |
| BLD129 | VH | PRT | WO2018054813; SEQ ID NO: 18 | 7475 |
| BLD130 | VH | PRT | WO2018054813; SEQ ID NO: 22 | 7476 |
| BLD131 | VH | PRT | WO2018054813; SEQ ID NO: 24 | 7477 |
| BLD132 | VH | PRT | WO2018054813; SEQ ID NO: 26 | 7478 |
| BLD133 | VH | PRT | U.S. Pat. No. 8,080,243; SEQ ID NO: 23 | 7479 |
| BLD134 | VH | PRT | U.S. Pat. No. 8,080,243; SEQ ID NO: 22 | 7480 |
| BLD135 | VH | PRT | U.S. Pat. No. 8,080,243; SEQ ID NO: 20 | 7481 |
| BLD136 | VH | PRT | U.S. Pat. No. 8,080,243; SEQ ID NO: 21 | 7482 |
| BLD137 | VH | PRT | U.S. Pat. No. 8,080,243; SEQ ID NO: 54 | 7483 |
| BLD138 | VL | PRT | WO2018093766; SEQ ID NO: 52 | 7484 |
| BLD139 | VL | PRT | WO2018093766; SEQ ID NO: 42 | 7485 |
| BLD140 | VL | PRT | WO2018093766; SEQ ID NO: 44 | 7486 |
| BLD141 | VL | PRT | WO2018093766; SEQ ID NO: 36 | 7487 |
| BLD142 | VL | PRT | WO2018093766; SEQ ID NO: 32 | 7488 |
| BLD143 | VL | PRT | WO2018093766; SEQ ID NO: 62 | 7489 |
| BLD144 | VL | PRT | WO2018093766; SEQ ID NO: 48 | 7490 |
| BLD145 | VL | PRT | WO2018093766; SEQ ID NO: 58 | 7491 |
| BLD146 | VL | PRT | WO2018093766; SEQ ID NO: 34 | 7492 |
| BLD147 | VL | PRT | WO2018054813; SEQ ID NO: 25 | 7493 |
| BLD148 | VL | PRT | WO2018054813; SEQ ID NO: 17 | 7494 |
| BLD149 | VL | PRT | WO2018054813; SEQ ID NO: 21 | 7495 |
| BLD150 | VL | PRT | WO2018054813; SEQ ID NO: 23 | 7496 |
| BLD151 | VL | PRT | US20180134806; SEQ ID NO: 75 | 7497 |
| BLD152 | VL | PRT | U.S. Pat. No. 8,080,243; SEQ ID NO: 53 | 7498 |
| BLD153 | VL | PRT | U.S. Pat. No. 8,080,243; SEQ ID NO: 18 | 7499 |
| BLD154 | VL | PRT | U.S. Pat. No. 8,080,243; SEQ ID NO: 19 | 7500 |
| BLD155 | VL | PRT | U.S. Pat. No. 8,080,243; SEQ ID NO: 17 | 7501 |
| BLD156 | VL | PRT | U.S. Pat. No. 8,080,243; SEQ ID NO: 16 | 7502 |
| BLD157 | VL | PRT | U.S. Pat. No. 9,068,001; SEQ ID NO: 36 | 7503 |
| BLD158 | VL | PRT | U.S. Pat. No. 9,068,001; SEQ ID NO: 37 | 7504 |
| BLD159 | VL | PRT | U.S. Pat. No. 9,068,001; SEQ ID NO: 38 | 7505 |
| BLD160 | VL | PRT | U.S. Pat. No. 9,068,001; SEQ ID NO: 39 | 7506 |
| BLD161 | VL | PRT | U.S. Pat. No. 9,068,001; SEQ ID NO: 45 | 7507 |
| BLD162 | VL | PRT | U.S. Pat. No. 9,068,001; SEQ ID NO: 49 | 7508 |
| BLD163 | VL | PRT | U.S. Pat. No. 9,068,001; SEQ ID NO: 51 | 7509 |
| BLD164 | VH | PRT | U.S. Pat. No. 9,068,001; SEQ ID NO: 40 | 7510 |
| BLD165 | VH | PRT | U.S. Pat. No. 9,068,001; SEQ ID NO: 41 | 7511 |
| BLD166 | VH | PRT | U.S. Pat. No. 9,068,001; SEQ ID NO: 42 | 7512 |
| BLD167 | VH | PRT | U.S. Pat. No. 9,068,001; SEQ ID NO: 43 | 7513 |
| BLD168 | VH | PRT | U.S. Pat. No. 9,068,001; SEQ ID NO: 55 | 7514 |
| BLD169 | VH | PRT | U.S. Pat. No. 9,068,001; SEQ ID NO: 59 | 7515 |
| BLD170 | VH | PRT | U.S. Pat. No. 9,068,001; SEQ ID NO: 63 | 7516 |
| BLD171 | VH | PRT | U.S. Pat. No. 9,068,001; SEQ ID NO: 76 | 7517 |

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 5, or variants or fragments thereof. As another non-limiting example, the antibody may be one or more of the heavy chain sequences listed in Table 5. As a non-limiting example, the antibody may be one or more of the light chain sequences listed in Table 5, or variants or fragments thereof.

In some embodiments, the payload region of the AAV particle comprises a nucleic acid sequence encoding a polypeptide comprising a heavy chain and alight chain sequence listed in Table 5, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Table 5, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5 to 3′ direction, an antibody light chain sequence from Table 5, one or more linkers from Table 2 and a heavy chain sequence from Table 5.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5 to 3′ direction, an antibody heavy chain sequence from Table 5, one or more linkers from Table 2, and alight chain sequence from Table 5.

In some embodiments, the payload region comprises a nucleic acid sequence encoding a single heavy chain. As a non-limiting example, the heavy chain is an amino acid sequence or fragment thereof described in Table 5.

Shown in Table 5 are a listing of antibodies and their polynucleotides and/or polypeptides sequences. These sequences may be encoded by or included in the AAV particles of the present disclosure. Variants or fragments of the antibody sequences described in Table 5 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 5. CDRs of the antibodies encoded by the viral genomes of the present disclosure may be 50%, 60%, 70%, 80%, 90%, 95% identical to CDRs listed in or incorporated in the sequences of Table 5. Methods of determining CDRs are well known in the art and are described herein. Payload regions may encode antibody variants with one or more heavy chain variable domain (VW) or light chain variable domain (V) derived from the antibody sequences in Table 5. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any blood and blood vessel disease-associated antibodies, not limited to those described in Table 5, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the blood and blood vessel disease-associated antibodies as described in International Publication Number WO2017048614, WO2017048902, WO2017049024, WO2017049035, WO2017049251, WO2017050820, WO2017050974, WO2017051002, WO2017051888, WO2017053703, WO2017053807, WO2017053889, WO2017053932, WO2017055273, WO2017055547, WO2017055612, WO2017055613, WO2017055614, WO2017055617, WO2017055908, WO2017058859, WO2017058944, WO2017059380, WO2017059878, WO2017060247, WO2017060504, WO2017062016, WO2017062456, WO2017062496, WO2017062672, WO2017062748, WO2017062888, WO2017062966, WO2017065203, WO2017065493, WO2017066760, WO2017070170, WO2017070364, WO2017070395, WO2017070423, WO2017070476, WO2017070527, WO2017070561, WO2017070613, WO2017070616, WO2017070622, WO2017070623, WO2017070624, WO2017070626, WO2017072361, WO2017074013, WO2017075052, WO2017075119, WO2017075325, WO2017075432, WO2017076916, WO2017077391, WO2017079591, WO2017079768, WO2017079831, WO2017079832, WO2017079833, WO2017079834, WO2017079835, WO2017081066, WO2017083314, WO2017083371, WO2017083488, WO2017083515, WO2017083750, WO2017085035, WO2017086627, WO2017088028, WO2017088821, WO2017088974, WO2017089334, WO2017089895, WO2017091487, WO2017093410, WO2017093985, WO2017095088, WO2017095486, WO2017095487, WO2017095875, WO2017095918, WO2017099712, WO2017100289, WO2017100305, WO2017100540, WO2017100597, WO2017106326, WO2017106453, WO2017106504, WO2017106656, WO2017106684, WO2017106859, WO2017109721, WO2017112536, WO2017112762, WO2017112775, WO2017112877, WO2017112944, WO2017114230, WO2017114401, WO2017114972, WO2017114973, WO2017114975, WO2017114976, WO2017117384, WO2017118307, WO2017118761, WO2017119434, WO2017119435, WO2017120222, WO2017120261, WO2017120344, WO2017120525, WO2017120534, WO2017123557, WO2017123636, WO2017123644, WO2017123646, WO2017125586, WO2017127468, WO2017127833, WO2017128534, WO2017132578, WO2017133175, WO2017133633, WO2017133639, WO2017133640, WO2017133673, WO2017134234, WO2017134301, WO2017134302, WO2017134305, WO2017134306, WO2017135791, WO2017136313, WO2017136820, WO2017137542, WO2017137583, WO2017137954, WO2017138471, WO2017139212, WO2017139276, WO2017139623, WO2017139975, WO2017140256, WO2017141980, WO2017142832, WO2017143406, WO2017144681, WO2017144896, WO2017147247, WO2017147383, WO2017147742, WO2017149122, WO2017149143, WO2017149513, WO2017151783, WO2017152085, WO2017152102, WO2017153567, WO2017155355, WO2017156298, WO2017156488, WO2017157948, WO2017158116, WO2017160104, WO2017160699, WO2017160700, WO2017162791, WO2017163049, WO2017165245, WO2017165464, WO2017165614, WO2017165683, WO2017165734, WO217165766, WO2017165864, WO2017170597, WO2017170847, WO2017172990, WO2017173256, WO2017173328, WO2017173349, WO2017174017, WO2017174586, WO2017176651, WO2017176760, WO2017176864, WO2017177013, WO2017177175, WO2017177333, WO2017178493, WO2017180530, WO2017180555, WO2017180936, WO2017180993, WO2017181021, WO2017181031, WO2017181039, WO2017181109, WO2017181119, WO2017181139, WO2017182603, WO2017184201, WO2017184973, WO2017185037, WO2017186121, WO2017186908, WO2017188570, WO2017189279, WO2017189556, WO2017189813, WO2017189827, WO2017189959, WO2017189963, WO2017190001, WO2017190100, WO2017191062, WO2017192470, WO2017192668, WO2017193059, WO2017193107, WO2017193115, WO2017194265, WO2017194782, WO2017194783, WO2017196426, WO2017197259, WO2017197263, WO2017197347, WO2017197376, WO2017198148, WO2017198741, WO2017201325, WO2017201340, WO2017201731, WO2017202387, WO2017202879, WO2017203450, WO2017205536, WO2017205538, WO2017205651, WO2017205694, WO2017205721, WO2017206773, WO2017207574, WO2017207739, WO2017208210, WO2017210058, WO2017211313, WO2017211731, WO2017212462, WO2017214186, WO2017214207, WO2017214298, WO2017214338, WO2017214706, WO2017215585, WO2017215590, WO2017216724, WO2017218355, WO2017218515, WO2017218533, WO2017218824, WO2017218825, WO2017219025, WO217219029, WO2017219093, WO2017219687, WO2017219690, WO2017221128, WO2017222330, WO2017222337, WO2017223283, WO2017223284, WO2017223286, WO2018004283, WO2018005276, WO2018005551, WO2018005556, WO2018005559, WO2018005697, WO2018005904, WO2018006092, WO2018007258, WO2018007314, WO2018007570, WO2018007922, WO2018007923, WO2018007924, WO2018007999, WO2018009624, WO2018009732, WO2018009811, WO2018009894, WO2018009921, WO2018010140, WO2018010789, WO2018011073, WO2018011283, WO2018011344, WO2018011353, WO2018011421, WO2018011584, WO2018013563, WO2018013917, WO2018013918, WO2018014126, WO2018016884, WO2018017714, WO2018017797, WO2018017996, WO2018022762, WO2018022786, WO2018023100, WO2018023121, WO2018023136, WO2018026018, WO2018026722, WO2018026969, WO2018027155, WO2018029124, WO2018030806, WO2018031408, WO2018031424, WO2018032638, WO2018035001, WO2018035107, WO2018035119, WO2018035210, WO2018036852, WO2018037000, WO2018037001, WO2018038243, WO2018039068, WO2018044105, WO2018044619, WO2018044640, WO2018044903, WO2018045018, WO2018045054, WO2018045325, WO2018047154, WO2018047894, WO2018048234, WO2018049053, WO2018049474, WO2018050027, WO2018050028, WO2018050783, WO2018052153, WO2018052827, WO2018053029, WO2018053597, WO2018054240, WO2018054241, WO2018057823, WO2018058002, WO2018064205, WO2018064602, WO2018064611, WO2018065552, WO2018069500, WO2018069927, WO2018070390, WO2018070479, WO2018071500, WO2018071624, WO2018071796, WO2018071871, WO2018071873, WO2018071913, WO2018073363, WO2018073680, WO2018075378, WO2018075408, WO2018075600, WO2018075621, WO2018075792, WO2018075794, WO2018075807, WO2018075813, WO2018075820, WO2018075960. WO2018075980, WO2018076024, WO2018077208, WO2018077775, WO2018077893, WO2018081282, WO2018081370, WO2018081448, WO2018081649, WO2018081754, WO2018081755, WO2018081832, WO2018081897, WO2018081898, WO2018083071, WO2018083248, WO2018083633, WO2018083692, WO2018084236, WO2018084836, WO2018085400. WO2018085599, WO2018085731, WO2018086585, WO2018086605, WO2018087644, WO2018088850, WO2018089305, WO2018089532, WO2018089890, WO2018090057, WO2018091661, WO2018093766, WO2018094021, WO2018094112, WO2018094144, WO2018094282, WO2018098277, WO2018099614, WO2018100036, WO2018101470, WO2018102589, WO2018102597, WO2018102612, WO2018102682, WO2018102785, WO2018102786, WO2018102787, WO2018102795, WO2018104554, WO2018104562, WO2018105560, WO2018106645, WO2018106732, WO2018106776, WO2018106781, WO2018106862, WO2018107058, WO2018107069, WO2018109228, WO2018109588, WO2018109663, WO2018112426, WO2018112549, WO2018113781, WO2018115225, WO2018115319, WO2018115885, WO2018115887, WO2018116178, WO2018116255, WO2018116267, WO2018118713, WO2018118754, WO2018119001, WO2018119171, WO2018119279, WO2018122053, WO2018124107, WO2018124121, WO2018124277, WO2018126259, WO2018126317, WO2018127610, WO2018127787, WO2018127791, WO2018128779, WO2018128939, WO2018128973, WO2018129329, WO2018129395, WO2018129400, WO2018129533, WO2018130513, WO2018130657, WO2018132423, WO2018133837, WO2018134691, WO2018134787, WO2018134817, WO2018135653, WO2018136163, WO2018136553, WO2018136562, WO2018136570, WO2018136825, WO2018136910, WO2018137705, WO2018138297, WO2018138681, WO2018138709, WO2018139404, WO2018140242, WO2018140510, WO2018140660, WO2018140725, WO2018140729, WO2018140733, WO2018140831, WO2018141863, WO2018141964, WO2018142322, WO2018144410, WO2018144425, WO2018144999, WO2018145075, WO2018145125, WO2018146074, WO2018146253, WO2018146549, WO2018146594, WO2018147245, WO2018148224, WO2018148476, WO2018151816, WO2018152181, WO2018152359, WO2018152687, WO2018153340, WO2018153372, WO2018154392, WO2018156494, WO2018157169, WO2018158632, WO2018159845, WO2018160731, WO2018160841, WO2018160897, WO2018161017, WO2018162749, WO2018164441, WO2018165062, WO2018165089, WO2018165228, WO2018165475, WO2018166468, WO2018166495, WO2018167267, WO2018168768, WO2018168769, WO2018168779, WO2018169948, WO2018169993, WO2018170145, WO2018170188, WO2018170256, WO2018170338, WO2018170351, WO2018174274, WO2018174408, WO2018175319, WO2018175476, WO2018175752, WO2018175790, WO2018175833, WO2018176132, WO2018176844, WO2018177967, WO2018178047, WO2018178122, WO2018178123, WO2018178307, WO2018178352, WO2018178354, WO2018181870, WO2018181935, WO2018182284, WO2018183216, WO2018183219, WO2018183459, WO2018185110, WO2018185232, WO2018185709, WO2018187332, WO2018187350, WO2018187613, WO2018187682, WO2018187791, WO2018187799, WO2018188331, WO2018191479, WO2018191502, WO2018191548, WO2018191660, WO2018191718, WO2018191723, WO2018193427, WO2018193457, WO2018195008, WO2018195034, WO2018195283, WO2018195418, WO2018195506, WO2018195912, WO2018197492, WO2018200422, WO2018200818, WO2018201014, WO2018201087, WO2018203289, WO2018204363, WO2018204427, WO2018204520, WO2018204677, WO2018204679, WO2018204757, WO2018204868, WO2018204871, WO2018204895, WO2018205917, WO2018208091, WO2018208121, WO2018208625, WO2018208670, WO2018208709, WO2018208856, WO2018209344, WO2018213297, WO2018213316, WO2018213331, WO2018213430, WO2018213592, WO2018213679, WO2018215571, WO2018215614, WO2018215768, WO2018217116, WO2018217227, WO2018218083, WO2018218185, WO2018218215, WO2018218219, WO2018218222, WO2018218240, WO2018218352, WO2018220584, WO2018221521, WO2018222019, WO2018222433, WO2018222901, WO2018222935, WO2018223051, WO2018223098, WO2018223101, WO2018223182, WO2018224550, WO2018225041, WO2018226339, WO2018226580, WO2018226776, WO2018226833, WO2018228406, WO2018229193, WO2018229194, WO2018229195, WO2018229197, WO2018229303, WO2018229612, WO2018231254, WO2018232088, WO2018232188, WO2018232278, WO2018232366, WO2018232467, WO2018233333, WO2018234383, WO2018234575, WO2018234576, WO2018237064, WO2018237157, WO2018237173, WO2018237192, WO2018237287, WO2018237338, WO2019000620, WO2019001560, WO2019003074, WO2019003162, WO2019003164, WO2019004831, WO2019005634, WO2019005635, WO2019005636, WO2019005638, WO2019005756, WO2019005847, WO2019006280, WO2019008123, WO2019009231, WO2019009407, WO2019009419, WO2019009879, WO2019010219, WO2019010224, WO2019010486, WO2019011167, WO2019012014, WO2019013392, WO2019013394, WO2019014360, WO2019014623, WO2019014768, WO2019018757, WO2019018828, WO2019023056, WO2019023148, WO2019023347, WO2019023396, WO2019023410, WO2019023482, WO2019023564, WO2019025484, WO2019025908, WO2019027903, WO2019028283, WO2019028292. WO2019028427, WO2019028530, WO2019030757, WO2019031938, WO2019031939, WO2019032661, WO2019032662, WO2019032663, WO2019032927, WO2019032929, WO2019032945, WO2019032955, WO2019033046, WO2019033057, WO2019035005, WO2019035034, WO2019036460, WO2019040390, WO2019040471, WO2019040649, WO2019042119, WO2019042285, WO2019042889, WO2019043065, WO2019045452, WO2019045856, WO2019046225, WO2019046321, WO2019046600, WO2019046817, WO2019047921, WO2019047932, WO2019049967, WO2019050326, WO2019050935, WO2019051132, WO2019051335, WO2019051397, WO2019051470, WO2019052562, WO2019054460, WO2019054819, WO2019055399, WO2019055537, WO2019055631, WO2019055841, WO2019056099, WO2019057102, WO2019057992, WO2019060653, WO2019062831, WO2019062871, WO2019065795, WO2019066617, WO2019066620, WO2019067499, WO2019067540, WO2019067805, WO2019069229, WO2019070577, WO2019070680, WO2019070714, WO2019070740, WO2019071205, WO2019074333, WO2019074912, WO2019075270, WO2019075385, WO2019075413, WO2019075519, WO2019078697, WO2019079569, WO2019079772, WO2019079809, WO2019081595, WO2019081692, WO2019081902, WO2019084024, WO2019084053, WO2019084057, WO2019084060, WO2019084064, WO2019084067, WO2019084249, WO2019084307, WO2019084332, WO2019084438, WO2019084460, WO2019084692, WO2019085804, WO2019086512. WO2019086580, WO2019087087, WO2019087115, WO2019087151, WO2019088658, WO2019089395, WO2019089473, WO2019089592, WO2019089610, WO2019089755, WO2019089848, WO2019089855, WO2019089858, WO2019090002, WO2019090003, WO2019090004, WO2019090069, WO2019090074, WO2019090076, WO2019090078, WO2019090081, WO2019090082, WO2019090085, WO2019090088, WO2019090090, WO2019090134, WO2019090263, WO2019091384, WO2019092505, WO2019092677, WO2019094533, WO2019094576, WO2019094578, WO2019094595, WO2019094608, WO2019094700, WO2019094938, WO2019094983, WO2019097244, WO2019099479, WO2019099483, WO2019099639, WO2019099744, WO2019099993, WO2017180936, WO2019088143, WO2019046912, WO2019035055, WO2019012019, WO2018234543, WO2018224439, WO2018211529, WO2018156741, WO2018135501, WO2018113788, WO2018111010, WO2018054813, WO2018022844, WO2018021450, WO2017219034, and WO2017076967, the contents of each of which are herein incorporated by reference in their entirety.

In some embodiments, payloads may encode blood and blood vessel disease-associated antibodies (or fragments thereof) as described in Arenas-Ramirez et al, (Sci Transl Med, November 2016, Vol. 8(367), p 367ra166; the contents of which are herein incorporated by reference in their entirety). Such embodiments may include antibody NARA1 or fragments thereof.

In some embodiments, payloads may encode blood and blood vessel disease-associated antibodies (or fragments thereof) as described in Liu et al. (Molecular Therapy; February 2018, Vol. 26(2), p. 542-549, the contents of which are herein incorporated by reference in their entirety). Such embodiments may include antibody AAV8-antiVEGFfab (RGX-314) or fragments thereof. In some embodiments, antibody AAV8-antVEGFfab (RGX-314) may be used for the treatment of patients suffering from neovascular age-related macular degeneration (NVAMD). In some embodiments, payloads may encode VEGF associated antibodies (or fragments thereof) described in International Publication WO2017180936; the contents of which are herein incorporated by reference in their entirety. The payload region may encode a heavy chain antibody, such as, but not limited to, SEQ ID NO: 2 of International Publication Number WO2017180936. In some embodiments, the payload region may encode alight chain antibody, such as, but not limited to, SEQ ID NO: 1 of International Publication Number WO2017180936. In some embodiments, the payload may be SEQ ID NO: 24, 35-44 of International Publication Number WO2017180936. Such embodiments may include antibody Ranibizumab or fragments.

In some embodiments, payloads may encode blood and blood vessel disease-associated antibodies (or fragments thereof) taught in U.S. Pat. No. 8,080,243, the contents of which are herein incorporated by reference in their entirety. Such embodiments may include antibody Bococizumab or fragments thereof. In certain embodiments, the payload region encodes antibody Bococizumab or fragments thereof selected from SEQ ID NO: 53-54, as described in U.S. Pat. No. 8,080,243.

In some embodiments, payloads may encode blood and blood vessel disease-associated antibodies (or fragments thereof) taught in International Publication Number WO201805413, the contents of which are herein incorporated by reference in their entirety. Such embodiments may include antibodies 1A6, 1A6, TPP-3238, TPP-3290, TPP-3577, TPP-3583, or fragments thereof. In certain embodiments, the payload region encodes antibodies 1A6, 1A6, TPP-3238, TPP-3290, TPP-3577, TPP-3583, or fragments thereof selected from SEQ ID NO: 21-38, as described in WO2018054813.

In some embodiments, payloads may encode blood and blood vessel disease-associated antibodies (or fragments thereof) taught in International Publication Number WO2018093766, the contents of which are herein incorporated by reference in their entirety. Such embodiments may include antibodies 6C12, TPP-4935, TPP-5906, TPP-5907, TPP-7776, TPP-7777, TPP-7778, TPP-7779, TPP-7781, TPP-7782, TPP-7783, TPP-7787, TPP-7788, TPP-7789, TPP-7790, TPP-7791, TPP-7792, TPP-7793, TPP-7794, TPP-7795, TPP-7796, TPP-7797, or fragments thereof. In certain embodiments, the payload region antibodies 6C12, TPP-4935, TPP-5906, TPP-5907, TPP-7776, TPP-7777, TPP-7778, TPP-7779, TPP-7781, TPP-7782, TPP-7783, TPP-7787, TPP-7788, TPP-7789, TPP-7790, TPP-7791, TPP-7792, TPP-7793, TPP-7794, TPP-7795, TPP-7796, TPP-7797, or fragments thereof selected from SEQ ID NO: 16-67, as described in WO2018093766.

In some embodiments, payloads may encode blood and blood vessel disease-associated antibodies (or fragments thereof) taught in International Publication Number WO2018134184, the contents of which are herein incorporated by reference in their entirety. Such embodiments may include antibody BAY1213790, or fragments thereof. In certain embodiments, the payload region antibody BAY1213790, or fragments thereof selected from SEQ ID NO: 1-2, as described in WO2018134184.

In some embodiments, payloads may encode blood and blood vessel disease-associated antibodies (or fragments thereof) taught in US Patent Publication Number US20180134806, the contents of which are herein incorporated by reference in their entirety. Such embodiments may include antibody CSL312 or 3F7 Factor XIIa antagonist monoclonal antibody or fragments thereof. In certain embodiments, the payload region encodes antibody CSL312 or 3F7 Factor XIIa antagonist monoclonal antibody or fragments thereof selected from SEQ ID NO: 6-77, as described in US20180134806.

### Antibodies for the Treatment of Respiratory Diseases

In some embodiments, the payload region of the AAV particle comprises one or more nucleic acid sequences encoding respiratory disease-associated antibodies, variants or fragments thereof.

In some embodiments, the payload region of the AAV particle comprises one or more nucleic acid sequences encoding one or more of the payload antibody polypeptides listed in Table 6, or variants or fragments thereof.

In some embodiments, the payload region of the AAV particle comprises one or more nucleic acid sequences listed in Table 6 or variants or fragments thereof.

In some embodiments, the payload region of the AAV particle comprises a nucleic acid sequence encoding a payload antibody with at least 50% identity to one or more payload antibody polypeptides listed in Table 6. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 6, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 6, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 6, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%,63%,64%, 65%, 66%,67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 6, or variants or fragments thereof.

In some embodiments, the light chain of the encoded antibody polypeptide may have 50%, 51%, 52%,53%, 54%, 55%, 56%, 57%, 58%, 59%, 60%, 61%, 62%,63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 6, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 6, or variants or fragments thereof.

In some embodiments, the payload antibody has 90% identity to one or more of the antibody polypeptides listed in Table 6, or variants or fragments thereof.

In some embodiments, the payload antibody has 91% identity to one or more of the antibody polypeptides listed in Table 6, or variants or fragments thereof.

In some embodiments, the payload antibody has 92% identity to one or more of the antibody polypeptides listed in Table 6, or variants or fragments thereof.

In some embodiments, the payload antibody has 93% identity to one or more of the antibody polypeptides listed in Table 6, or variants or fragments thereof.

In some embodiments, the payload antibody has 94% identity to one or more of the antibody polypeptides listed in Table 6, or variants or fragments thereof.

In some embodiments, the payload antibody has 95% identity to one or more of the antibody polypeptides listed in Table 6, or variants or fragments thereof.

In some embodiments, the payload antibody has 96% identity to one or more of the antibody polypeptides listed in Table 6, or variants or fragments thereof.

In some embodiments, the payload antibody has 97% identity to one or more of the antibody polypeptides listed in Table 6, or variants or fragments thereof.

In some embodiments, the payload antibody has 98% identity to one or more of the antibody polypeptides listed in Table 6, or variants or fragments thereof.

In some embodiments, the payload antibody has 99% identity to one or more of the antibody polypeptides listed in Table 6, or variants or fragments thereof.

In some embodiments, the payload antibody has 100% identity to one or more of the antibody polypeptides listed in Table 6, or variants or fragments thereof.

In some embodiments, the payload region of the AAV particle comprises a nucleic acid sequence with at least 50% identity to one or more nucleic acid sequences listed in Table 6, or variants or fragments thereof. The payload nucleic acid sequence may have 50%, 51%, 52%, 53%, 54%, 55%, 56%, 57%, 58%, 59%, 60%, 61%, 62%, 63%, 64%, 65%,6%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Tables 6, or variants or fragments thereof.

In some embodiments, the payload nucleic acid sequence has 90% identity to one or more of the nucleic acid sequences listed in Table 6, or variants or fragments thereof.

In some embodiments, the payload nucleic acid sequence has 91% identity to one or more of the nucleic acid sequences listed in Table 6, or variants or fragments thereof.

In some embodiments, the payload nucleic acid sequence has 92% identity to one or more of the nucleic acid sequences listed in Table 6, or variants or fragments thereof.

In some embodiments, the payload nucleic acid sequence has 93% identity to one or more of the nucleic acid sequences listed in Table 6, or variants or fragments thereof.

In some embodiments, the payload nucleic acid sequence has 94% identity to one or more of the nucleic acid sequences listed in Table 6, or variants or fragments thereof.

In some embodiments, the payload nucleic acid sequence has 95% identity to one or more of the nucleic acid sequences listed in Table 6, or variants or fragments thereof.

In some embodiments, the payload nucleic acid sequence has 96% identity to one or more of the nucleic acid sequences listed in Table 6, or variants or fragments thereof.

In some embodiments, the payload nucleic acid sequence has 97% identity to one or more of the nucleic acid sequences listed in Table 6, or variants or fragments thereof.

In some embodiments, the payload nucleic acid sequence has 98% identity to one or more of the nucleic acid sequences listed in Table 6, or variants or fragments thereof.

In some embodiments, the payload nucleic acid sequence has 99% identity to one or more of the nucleic acid sequences listed in Table 6, or variants or fragments thereof.

In some embodiments, the payload nucleic acid sequence has 100% identity to one or more of the nucleic acid sequences listed in Table 6, or variants or fragments thereof.

In some embodiments, the payload antibody may be variants of any of the antibody polypeptides listed in Table 6, that exclude one or more amino acids designated as “X” or “x” in the described polypeptide sequence, wherein X may represent any amino acid. In some embodiments, the payload nucleic acid sequence may be variants of any of the nucleic acid sequences listed in Table 6, that exclude one or more nucleic acids designated as “n” or “N” in the described nucleic acid sequence, wherein n may represent any nucleic acid.

TABLE 6
Respiratory disease antibodies

| | | Type | | SEQ |
|---|---|---|---|---|
| Ab ID | Component | (PRT/DNA) | Reference | ID NO |
| RSP1 | HC | PRT | US20120027754; SEQ ID NO: 1 | 7518 |
| RSP2 | HC | PRT | US20120027754; SEQ ID NO: 3 | 7519 |
| RSP3 | HC | PRT | US20120027754; SEQ ID NO: 78 | 7520 |
| RSP4 | HC | PRT | US20120027754; SEQ ID NO: 79 | 7521 |
| RSP5 | HC | PRT | US20120027754; SEQ ID NO: 80 | 7522 |
| RSP6 | HC | PRT | US20120027754; SEQ ID NO: 81 | 7523 |
| RSP7 | HC | PRT | US20120027754; SEQ ID NO: 144 | 7524 |
| RSP8 | HC | PRT | US20120027754; SEQ ID NO: 145 | 7525 |
| RSP9 | HC | PRT | WO2017049024; SEQ ID NO: 60 | 7526 |
| RSP10 | LC | PRT | US20120027754; SEQ ID NO: 2 | 7527 |
| RSP11 | LC | PRT | US20120027754; SEQ ID NO: 4 | 7528 |
| RSP12 | LC | PRT | US20120027754; SEQ ID NO: 75 | 7529 |
| RSP13 | LC | PRT | US20120027754; SEQ ID NO: 76 | 7530 |
| RSP14 | LC | PRT | US20120027754; SEQ ID NO: 77 | 7531 |
| RSP15 | LC | PRT | US20120027754; SEQ ID NO: 86 | 7532 |
| RSP16 | LC | PRT | US20120027754; SEQ ID NO: 92 | 7533 |
| RSP17 | LC | PRT | US20120027754; SEQ ID NO: 93 | 7534 |
| RSP18 | LC | PRT | US20120027754; SEQ ID NO: 98 | 7535 |
| RSP19 | LC | PRT | US20120027754; SEQ ID NO: 135 | 7536 |
| RSP20 | LC | PRT | US20120027754; SEQ ID NO: 136 | 7537 |
| RSP21 | LC | PRT | US20120027754; SEQ ID NO: 137 | 7538 |
| RSP22 | LC | PRT | US20120027754; SEQ ID NO: 138 | 7539 |
| RSP23 | LC | PRT | US20120027754; SEQ ID NO: 139 | 7540 |
| RSP24 | LC | PRT | US20120027754; SEQ ID NO: 140 | 7541 |
| RSP25 | LC | PRT | US20120027754; SEQ ID NO: 141 | 7542 |
| RSP26 | LC | PRT | US20120027754; SEQ ID NO: 142 | 7543 |
| RSP27 | LC | PRT | WO2017049024; SEQ ID NO: 61 | 7544 |
| RSP28 | VH | PRT | US20120027754; SEQ ID NO: 82 | 7545 |
| RSP29 | VH | PRT | US20120027754; SEQ ID NO: 83 | 7546 |
| RSP30 | VH | PRT | US20120027754; SEQ ID NO: 84 | 7547 |
| RSP31 | VH | PRT | US20120027754; SEQ ID NO: 85 | 7548 |
| RSP32 | VH | PRT | US20120027754; SEQ ID NO: 94 | 7549 |
| RSP33 | VH | PRT | US20120027754; SEQ ID NO: 95 | 7550 |
| RSP34 | VH | PRT | US20120027754; SEQ ID NO: 96 | 7551 |
| RSP35 | VH | PRT | US20120027754; SEQ ID NO: 97 | 7552 |
| RSP36 | VH | PRT | WO2017049024; SEQ ID NO: 1 | 7553 |
| RSP37 | VH | PRT | WO2017049024; SEQ ID NO: 3 | 7554 |
| RSP38 | VL | PRT | US20120027754; SEQ ID NO: 87 | 7555 |
| RSP39 | VL | PRT | US20120027754; SEQ ID NO: 88 | 7556 |
| RSP40 | VL | PRT | US20120027754; SEQ ID NO: 89 | 7557 |
| RSP41 | VL | PRT | US20120027754; SEQ ID NO: 90 | 7558 |
| RSP42 | VL | PRT | US20120027754; SEQ ID NO: 91 | 7559 |
| RSP43 | VL | PRT | US20120027754; SEQ ID NO: 99 | 7560 |
| RSP44 | VL | PRT | US20120027754; SEQ ID NO: 100 | 7561 |
| RSP45 | VL | PRT | US20120027754; SEQ ID NO: 134 | 7562 |
| RSP46 | VL | PRT | WO2017049024; SEQ ID NO: 2 | 7563 |
| RSP47 | VL | PRT | WO2017049024; SEQ ID NO: 4 | 7564 |
| RSP48 | VL | PRT | WO2017049024; SEQ ID NO: 5 | 7565 |
| RSP49 | VL | PRT | WO2017049024; SEQ ID NO: 6 | 7566 |
| RSP50 | VL | PRT | WO2017049024; SEQ ID NO: 7 | 7567 |
| RSP51 | VL | PRT | WO2017049024; SEQ ID NO: 8 | 7568 |
| RSP52 | VL | PRT | WO2017049024; SEQ ID NO: 9 | 7569 |
| RSP53 | VL | PRT | WO2017049024; SEQ ID NO: 10 | 7570 |
| RSP54 | VL | PRT | WO2017049024; SEQ ID NO: 11 | 7571 |
| RSP55 | VL | PRT | WO2017049024; SEQ ID NO: 12 | 7572 |
| RSP56 | VL | PRT | WO2017049024; SEQ ID NO: 13 | 7573 |
| RSP57 | VL | PRT | WO2017049024; SEQ ID NO: 14 | 7574 |

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 6, or variants or fragments thereof. As another non-limiting example, the antibody may be one or more of the heavy chain sequences listed in Table 6. As a non-limiting example, the antibody may be one or more of the light chain sequences listed in Table 6, or variants or fragments thereof.

In some embodiments, the payload region of the AAV particle comprises a nucleic acid sequence encoding a polypeptide comprising a heavy chain and alight chain sequence listed in Table 6, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Tables 6, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 6, one or more linkers from Table 2 and a heavy chain sequence from Table 6.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 6, one or more linkers from Table 2, and alight chain sequence from Table 6.

In some embodiments, the payload region comprises a nucleic acid sequence encoding a single heavy chain. As a non-limiting example, the heavy chain is an amino acid sequence or fragment thereof described in Table 6.

Shown in Table 6 are a listing of antibodies and their polynucleotides and/or polypeptides sequences. These sequences may be encoded by or included in the AAV particles of the present disclosure. Variants or fragments of the antibody sequences described in Tables 6 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Tables 6. CDRs of the antibodies encoded by the viral genomes of the present disclosure may be 50%, 60%,70%, 80%, 90%, 95% identical to CDRs listed in or incorporated in the sequences of Tables 6. Methods of determining CDRs are well known in the art and are described herein. Payload regions may encode antibody variants with one or more heavy chain variable domain (V_{H}) or light chain variable domain (V_{L}) derived from the antibody sequences in Tables 6. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

In some embodiments, payloads may encode respiratory disease-associated antibodies (or fragments thereof) taught in US Publication Number US20120027754, the contents of which are herein incorporated by reference in their entirety. Such embodiments may include antibody BG00011 (STX-100) or fragments thereof. In certain embodiments, the payload region encodes antibody BG00011 (STX-100) or fragments thereof such as but not limited to SEQ ID NO: 7518.7524; 7527.7543; 7545-7552; and/or 7555-7562 described herein.

Payload regions of the viral genomes may encode any respiratory disease-associated antibodies, not limited to those described in Table 6, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the respiratory disease-associated antibodies as described in International Publication Number WO2016059622, WO2017046658, WO2017048593, WO2017048614, WO2017049024, WO2017049035, WO2017049139, WO2017049149, WO2017049251, WO2017049296, WO2017051888, WO2017053170, WO2017053250, WO2017053423, WO2017053469, WO2017053748, WO2017053807, WO2017053856, WO2017054646, WO2017055291, WO2017055547, WO2017055612, WO2017055613, WO2017055614, WO2017058780, WO2017058866, WO2017059243, WO2017059252, WO2017059380, WO2017059557, WO2017060322, WO2017062016, WO2017062456, WO2017062615, WO2017062619, WO2017062792, WO2017062820, WO2017062888, WO2017062952, WO2017062966, WO2017065493, WO2017068186, WO2017069628, WO2017070423, WO2017070456, WO2017070460, WO2017070475, WO2017070476, WO2017070622, WO2017070626, WO2017070649, WO2017070654, WO2017071625, WO2017072361, WO2017072366, WO2017072662, WO2017072757, WO2017074013, WO2017074074, WO2017075124, WO2017075188, WO2017075432, WO2017075615, WO2017076308, WO2017077391, WO2017079112, WO2017079115, WO2017079116, WO2017079117, WO2017079121, WO2017079369, WO2017079520, WO2017083296, WO2017083515, WO2017083582, WO2017084078, WO2017084495, WO2017087547, WO2017087587, WO2017087588, WO2017087589, WO2017087678, WO2017087800, WO2017087921, WO2017088734, WO2017088974, WO2017089447, WO2017091429, WO2017091487, WO2017091577, WO2017091719, WO2017092645, WO2017093448, WO2017093478, WO2017093844, WO2017095486, WO2017095487, WO2017095744, WO2017095823, WO2017095875, WO2017095918 WO2017096017, WO2017096026, WO2017096051, WO2017096163, WO2017096179, WO2017096182, WO2017096189, WO2017096276, WO2017096281, WO2017096397, WO2017099362, WO2017099712, WO2017100289, WO2017100540, WO2017100597, WO217106061, WO2017106129, WO2017106352, WO2017106453, WO2017106566, WO2017106609, WO2017106656, WO2017106684, WO2017106810, WO2017109496, WO2017112536, WO2017112741, WO2017112803, WO2017112811, WO2017112829, WO2017112877, WO2017112944, WO2017112954, WO2017112955, WO2017112956, WO2017117202, WO2017117384, WO2017117430, WO2017118321, WO2017118675, WO2017118841, WO2017119434, WO2017119435, WO2017119811, WO2017120280, WO2017120344, WO2017120534, WO2017120557, WO2017120599, WO2017120612, WO2017120996, WO2017120997, WO2017120998, WO2017121771, WO2017121905, WO2017122666, WO2017123556, WO2017123557, WO2017123646, WO2017123978, WO2017124001, WO2017124002, WO2017125532, WO2017125830, WO2017125831, WO2017127468, WO2017127499, WO2017127664, WO2017127933, WO2017130076, WO2017132457, WO2017132459, WO2017132555, WO2017132562, WO2017132827, WO2017133540, WO2017133673, WO2017134197, WO2017134234, WO2017134301, WO2017134302, WO2017134305, WO2017134306, WO2017136350, WO2017136562, WO217136607, WO2017137542, WO2017137954, WO2017139623, WO2017139975, WO2017140256, WO2017142294, WO2017142928, WO2017143270, WO2017144681, WO2017147368, WO2017147383, WO2017148424, WO2017149394, WO2017149513, WO2017149515, WO2017151176, WO2017151979, WO2017152076, WO2017152085, WO2017152088, WO217152102, WO2017153402, WO2017153953, WO2017153955, WO2017155355, WO2017156032, WO2017156479, WO2017156488, WO2017156500, WO2017157305, WO2017158116, WO2017158436, WO2017159287, WO2017159699, WO2017160587, WO2017160754, WO2017161206, WO2017161414, WO2017161976, WO2017162555, WO2017162791, WO2017165398, WO2017165464, WO2017165742, WO2017165766, WO2017165778, WO2017167967, WO2017171373, WO2017172260, WO2017172518, WO2017172990, WO2017173328, WO2017174568, WO2017175018, WO2017175054, WO2017175058, WO2017176864, WO2017177013, WO2017177169, WO2017177179, WO2017177955, WO2017178493, WO2017180536, WO2017180813, WO2017180864, WO2017180904, WO2017180993, WO2017181031, WO2017181034, WO2017181039, WO2017181049, WO2017181109, WO2017181119, WO2017181139, WO2017181420, WO2017182672, WO2017184942, WO2017185037, WO2017185177, WO2017186121, WO2017186182, WO2017186908, WO2017186928, WO2017187307, WO2017189279, WO2017189588, WO2017189959, WO2017189963, WO2017189964, WO2017190001, WO2017190025, WO2017190100, WO2017191062, WO2017191108, WO2017191325, WO2017193032, WO2017193059, WO2017193096, WO2017193107, WO2017194568, WO2017194782, WO2017194783, WO2017196263, WO2017196764, WO2017196847, WO2017196867, WO2017197199, WO2017197259, WO2017197331, WO2017197376, WO2017198148, WO2017198212, WO2017200493, WO2017201131, WO2017201204, WO2017201340, WO2017201440, WO2017201442, WO2017201731, WO2017202387, WO2017203450, WO2017204349, WO2017205216, WO2017205465, WO2017205536, WO2017205538, WO2017205694, WO2017205726, WO2017205738, WO2017205767, WO2017205875, WO2017206840, WO2017207477, WO2017207480, WO2017210058, WO2017210844, WO2017211873, WO2017212250, WO2017212442, WO2017214151, WO2017214182, WO2017214186, WO2017214282, WO2017214301, WO2017214335, WO2017214456, WO2017214458, WO2017214462, WO2017214706, WO2017215585, WO2017215590, WO2017218355, WO2017218435, WO2017218515, WO2017219025, WO2017219029, WO2017219687, WO2017219690, WO2017219995, WO2017220569, WO2017220988, WO2017220989, WO2017220990, WO2017223284, WO2018002339, WO2018005276, WO2018005519, WO2018005551, WO2018005556, WO2018005559, WO2018005697, WO2018005904, WO2018006785, WO2018006882, WO2018007592, WO2018007885, WO2018009507, WO2018009732, WO2018009811, WO2018010846, WO2018011421, WO2018013818, WO2018014122, WO2018014260, WO2018014855, WO2018014864, WO201015448, WO2018017673, WO2018017864, WO2018022762, WO2018023121, WO2018023136, WO2018025178, WO2018025982, WO2018026018, WO2018026248, WO2018026722, WO2018026748, WO2018026953, WO2018027025, WO2018027039, WO2018027084, WO2018027124, WO2018027203, WO2018027204, WO2018027252, WO2018029124, WO2018029284, WO2018029474, WO2018030806, WO2018031258, WO2018031408, WO2018031490, WO2018033135, WO2018033749, WO2018034434, WO2018035061, WO2018035107, WO2018035119, WO2018035210, WO2018035407, WO2018036438, WO2018036472, WO2018036473, WO2018036561, WO2018038583, WO2018038945, WO2018039020, WO2018039107 WO2018039274, WO2018039626, WO2018043311, WO2018044105, WO2018044640, WO2018045054, WO2018045090, WO2018045325, WO2018047894, WO2018048234, WO2018048975, WO2018049083, WO2018049118, WO2018049120, WO2018049474, WO2018050027, WO2018050028, WO2018050066, WO2018050111, WO2018050833, WO2018050852, WO2018052789, WO2018052818, WO2018053029, WO2018053032, WO2018053106, WO2018053434, WO2018053709, WO2018054353, WO2018054484, WO2018054973, WO2018057303, WO2018057669, WO2018057776, WO2018057823, WO2018057955, WO2018058001, WO2018059437, WO2018059465, WO2018059502, WO2018062402, WO2018064190, WO2018064255, WO2018064478, WO2018064611, WO2018066585, WO2018066626, WO2018067198, WO2018067331, WO2018067331, WO2018067993, WO2018068028, WO2018068282, WO2018068283, WO2018068336, WO2018068691, WO2018069467, WO2018069500, WO2018069871, WO2018070936, WO2018072743, WO2018073648, WO2018073680, WO2018075304, WO2018075375, WO2018075378, WO2018075591, WO2018075740, WO2018075813, WO2018075954 WO2018075960, WO2018075961, WO2018075974, WO2018075989, WO2018077893, WO2018079740, WO2018080812, WO2018081512, WO2018081648, WO2018082590, WO2018083248, WO2018083282, WO2018084836, WO2018085252, WO2018085358, WO2018085468, WO2018085469, WO2018085533, WO2018085555, WO2018085731, WO2018086139. WO2018086585, WO2018086605, WO2018087276, WO2018088877, WO2018088878, WO2018089293, WO2018089305, WO2018089532, WO2018089628, WO2018089829, WO2018090950, WO2018091661, WO2018091720, WO2018091739, WO2018091740, WO2018093866, WO2018094414, WO2018095428, WO2018097951, WO2018098035, WO2018098362, WO2018098365, WO2018099539, WO2018099968, WO2018099978, WO2018101448, WO2018102536, WO2018102589, WO2018102594, WO2018102597, WO2018102682, WO2018102746, WO2018102785, WO2018102787, WO2018102795, WO2018103884, WO2018104478, WO2018104483, WO2018106529, WO2018106588, WO2018106644, WO2018106645, WO2018106862, WO2018106864, WO2018107058, WO2018107069, WO2018107109, WO2018107134, WO2018110515, WO2018112334, WO2018112346, WO2018113258, WO2018113595, WO2018114728, WO2018115017, WO2018115051, WO2018115305, WO2018115319, WO2018115960, WO2018118754, WO2018118780, WO2018119118, WO2018119166, WO2018119171, WO2018119196, WO2018119314, WO2018119351, WO2018121473, WO2018121474, WO2018121475, WO2018121578, WO2018121580, WO2018121679, WO2018126233, WO2018126259, WO2018126595, WO2018127175, WO2018127586, WO2018127709, WO2018127710, WO2018127711, WO2018127713, WO2018127787, WO2018127791, WO2018128779, WO2018129007, WO2018129029, WO2018129261, WO2018129329, WO2018129331, WO2018129395, WO2018129400, WO2018129404, WO2018129451, WO2018129524, WO2018129533, WO2018129553, WO2018129559, WO2018133837, WO2018134681, WO2018135653, WO2018136163, WO2018136412, WO2018136825, WO2018137293, WO2018137294, WO2018137295, WO2018137598, WO2018138113, WO2018138297, WO2018138496, WO2018138521, WO2018139404, WO2018140242, WO2018140525, WO2018140725, WO2018140970, WO2018140973, WO2018141909, WO2018141959, WO2018141964, WO2018142322, WO2018144535, WO2018144999, WO2018145075, WO2018145120, WO2018145206, WO2018146074, WO2018146199, WO2018146253, WO2018146549, WO2018146612, WO2018147245, WO2018148180, WO2018148224, WO2018148383, WO2018148445, WO2018148447, WO2018148476, WO2018148585, WO2018148595, WO2018150029, WO2018150187, WO2018150326, WO2018151816, WO2018151820, WO2018151836, WO2018152518, WO2018152530, WO2018152687, WO2018153340, WO2018153372, WO2018154520, WO2018156494, WO2018156634, WO2018156740, WO2018157147, WO2018157169, WO2018158398, WO2018158658, WO2018159582, WO2018160538, WO2018160539, WO2018160704, WO2018160731, WO2018160841, WO2018160917, WO2018161092, WO2018161872, WO2018162430, WO2018162446, WO2018162724, WO2018162749, WO2018164441, WO2018165062, WO2018165228, WO2018165362, WO2018165619, WO2018165895, WO2018167322, WO2018168768, WO201816769, WO2018168779, WO2018169948, WO2018170338, WO2018174408, WO2018174544, WO2018174629, WO2018174984, WO2018175179, WO2018175403, WO2018175476, WO2018175740, WO2018175790, WO2018175833, WO2018175988, WO2018176132, WO2018177220, WO2018177324, WO2018178040, WO2018178046, WO2018178047, WO2018178122, WO2018178123, WO2018178354, WO2018178364, WO2018182266, WO2018182284, WO2018182422, WO2018182529, WO2018183041, WO2018183182, WO2018183366, WO2018183608, WO2018185050, WO2018185110, WO2018185232, WO2018185526, WO2018185709, WO2018187158, WO2018187191, WO2018187613, WO2018188047, WO2018189220, WO2018189381, WO2018191074, WO2018191414, WO2018191479, WO2018191545, WO2018191660, WO2018191718, WO2018194381, WO2018195226, WO2018195283, WO2018195418, WO2018197492, WO2018197675, WO2018198091, WO2018199176, WO2018199318, WO2018199593, WO2018200586, WO2018200812, WO2018200918, WO2018201014, WO2018201047, WO2018201051, WO2018201056, WO2018201096, WO2018202649, WO2018202794, WO2018204278, WO2018204303, WO2018204368, WO2018204677, WO2018204679, WO2018204872, WO2018205926, WO2018208121, WO2018208670, WO2018208856, WO2018209194, WO2018209701, WO2018210230, WO2018212136, WO2018213297, WO2018213316, WO2018213335, WO2018213592, WO2018213665, WO2018213680, WO2018215535, WO2018215571, WO2018215835, WO2018215935, WO2018215936, WO2018215937, WO2018215938, WO2018215964, WO2018217058, WO2018217227, WO2018217688, WO2018217799, WO2018217945, WO2018217947, WO2018218068, WO2018218083, WO2018218215, WO2018218240, WO2018219327, WO2018219901, WO2018220080, WO2018220100, WO2018220169, WO2018220546, WO2018221521, WO2018221969, WO2018222019, WO2018222675, WO2018222685, WO2018222689, WO2018222711, WO2018222718, WO2018222722, WO2018222770, WO2018222949, WO2018222962, WO2018223182, WO2018223923, WO2018223958, WO2018224550, WO2018224609, WO2018226339, WO2018226578, WO2018226833, WO2018227018, WO2018229193, WO2018229194, WO2018229195, WO2018229197, WO2018229612, WO2018229715, WO2018231827, WO2018232088, WO2018232144, WO2018232164, WO2018232188, WO2018232349, WO2018232372. WO2018232467, WO2018233333, WO2018234576, WO2018234793, WO2018235855, WO2018236728, WO2018236904, WO2018237010, WO2018237157, WO2018237173, WO2018237192, WO2018237287, WO2018237326, WO2018237335, WO2018237364, WO2019001559, WO2019003074, WO2019003159, WO2019003164, WO2019005208, WO2019005503, WO2019005634, WO2019005635, WO2019005636, WO2019005638, WO2019005756, WO2019005847, WO2019006007, WO2019006162, WO2019008377, WO2019008378, WO2019008379, WO2019009388, WO2019009879, WO2019010314, WO2019010566, WO2019011852, WO2019014091, WO2019014586, WO2019014623, WO2019015696, WO2019016371, WO2019016402, WO2019018640, WO2019018647, WO2019018730, WO2019018757, WO2019022187, WO2019023148, WO2019023460, WO2019023482, WO2019023661, WO2019024911, WO2019024933, WO2019025545, WO2019027721, WO2019027754, WO2019028051, WO2019028283, WO2019028555, WO2019029351, WO2019030706, WO2019031939, WO2019031965, WO2019032661, WO2019032662, WO2019032663, WO2019032699, WO2019032945, WO2019033057. WO2019034427, WO2019034779, WO2019034895, WO2019035630, WO2019035939, WO2019036724, WO2019039891, WO2019040348, WO2019040471, WO2019040608, WO2019040685, WO2019040727, WO2019040780, WO2019040808, WO2019042153, WO2019042226, WO2019042282, WO2019042285, WO2019043059, WO2019046321, WO2019046338, WO2019046652, WO2019047932, WO2019048040, WO2019048489, WO2019050326, WO2019050362, WO2019050935, WO2019051308, WO2019052562, WO2019054819, WO2019055537, WO2019055689, WO2019055842, WO2019056281, WO2019057100, WO2019057102, WO2019057124, WO2019057792, WO2019059411, WO2019059771, WO2019060418, WO2019060542, WO2019060653, WO2019062518, WO2019062642, WO2019062755, WO2019062871, WO2019062877, WO2019063802, WO2019065964, WO2019066535, WO2019067332, WO2019067491, WO2019067499, WO2019067805, WO2019067978, WO2019068904, WO2019068907, WO2019070013, WO2019070161, WO2019072220, WO2019073069, WO2019074124, WO2019075136, WO2019075270, WO2019075405, WO2019075413, WO2019075417, WO2019075433 WO2019075472, WO2019076277, WO2019077132, WO2019078600, WO2019078697, WO2019078698, WO2019079249, WO2019079569, WO2019079671, WO2019079762, WO2019079772, WO2019079809, WO2019079914, WO2019080858, WO2019080872, WO2019080883, WO2019080889, WO2019080909, WO2019080941, WO2019081595, WO2019081902 WO2019081983, WO2019083506, WO2019084057, WO2019084060, WO2019084064, WO2019084067, WO2019084249, WO2019084460, WO2019084553, WO2019084692, WO2019086573, WO2019086574, WO2019086878, WO2019087087, WO201908658, WO2019089473, WO2019089755, WO2019089921, WO2019089969, WO2019090002, WO2019090003, WO2019090069, WO2019090074, WO2019090076, WO2019090078, WO2019090081, WO2019090082, WO2019090085, WO2019090088, WO2019090090, WO2019090110, WO2019090134, WO2019090263, WO2019091384, WO2019092181, WO2019092451, WO2019092452, WO2019093807, WO2019094482, WO2019094578, WO2019094700, WO2019096900, WO2019097244, WO2019099440, WO2019099597, and WO2018081435, the contents of each of which are herein incorporated by reference in their entirety.

### Antibodies for the Treatment of Muscle Diseases

In some embodiments, the payload region of the AAV particle comprises one or more nucleic acid sequences encoding muscle disease-associated antibodies, variants or fragments thereof.

In some embodiments, the payload region of the AAV particle comprises one or more nucleic acid sequences encoding one or more of the payload antibody polypeptides listed in Tables 3-16, or variants or fragments thereof.

In some embodiments, the payload region of the AAV particle comprises one or more nucleic acid sequences listed in Tables 3-16, or variants or fragments thereof.

In some embodiments, the payload region of the AAV particle comprises a nucleic acid sequence encoding a payload antibody with at least 50% identity to one or more payload antibody polypeptides listed in Tables 3-16. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Tables 3-16, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Tables 3-16, or variants or fragments thereof.

In some embodiments, the variable region sequence(s) of the encoded antibody polypeptide may have 50%, 51%, 52%, 53%, 54%, 55%, 56%, 57%, 58%, 59%,60%,61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Tables 3-16, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%,87%, 88%, 89%, 90%, 91%, 92%, 93%, 94%, 95%, 96%, 97%, 98%, 99%, or 100% identity to one or more of the payload heavy chain antibody polypeptides listed in Tables 3-16, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Tables 3-16, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Tables 3-16, or variants or fragments thereof.

In some embodiments, the payload antibody has 90% identity to one or more of the antibody polypeptides listed in Tables 3-16, or variants or fragments thereof.

In some embodiments, the payload antibody has 91% identity to one or more of the antibody polypeptides listed in Tables 3-16, or variants or fragments thereof.

In some embodiments, the payload antibody has 92% identity to one or more of the antibody polypeptides listed in Tables 3-16, or variants or fragments thereof.

In some embodiments, the payload antibody has 93% identity to one or more of the antibody polypeptides listed in Tables 3-16, or variants or fragments thereof.

In some embodiments, the payload antibody has 94% identity to one or more of the antibody polypeptides listed in Tables 3-16, or variants or fragments thereof.

In some embodiments, the payload antibody has 95% identity to one or more of the antibody polypeptides listed in Tables 3-16, or variants or fragments thereof.

In some embodiments, the payload antibody has 96% identity to one or more of the antibody polypeptides listed in Tables 3-16, or variants or fragments thereof.

In some embodiments, the payload antibody has 97% identity to one or more of the antibody polypeptides listed in Tables 3-16, or variants or fragments thereof.

In some embodiments, the payload antibody has 98% identity to one or more of the antibody polypeptides listed in Tables 3-16, or variants or fragments thereof.

In some embodiments, the payload antibody has 99% identity to one or more of the antibody polypeptides listed in Tables 3-16, or variants or fragments thereof.

In some embodiments, the payload antibody has 100% identity to one or more of the antibody polypeptides listed in Tables 3-16, or variants or fragments thereof.

In some embodiments, the payload region of the AAV particle comprises a nucleic acid sequence with at least 50% identity to one or more nucleic acid sequences listed in Tables 3.16, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%,67%,68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Tables 3-16, or variants or fragments thereof.

In some embodiments, the payload nucleic acid sequence has 90% identity to one or more of the nucleic acid sequences listed in Tables 3-16, or variants or fragments thereof.

In some embodiments, the payload nucleic acid sequence has 91% identity to one or more of the nucleic acid sequences listed in Tables 3-16, or variants or fragments thereof.

In some embodiments, the payload nucleic acid sequence has 92% identity to one or more of the nucleic acid sequences listed in Tables 3-16, or variants or fragments thereof.

In some embodiments, the payload nucleic acid sequence has 93% identity to one or more of the nucleic acid sequences listed in Tables 3-16, or variants or fragments thereof.

In some embodiments, the payload nucleic acid sequence has 94% identity to one or more of the nucleic acid sequences listed in Tables 3-16, or variants or fragments thereof.

In some embodiments, the payload nucleic acid sequence has 95% identity to one or more of the nucleic acid sequences listed in Tables 3-16, or variants or fragments thereof.

In some embodiments, the payload nucleic acid sequence has 96% identity to one or more of the nucleic acid sequences listed in Tables 3-16, or variants or fragments thereof.

In some embodiments, the payload nucleic acid sequence has 97% identity to one or more of the nucleic acid sequences listed in Tables 3-16, or variants or fragments thereof.

In some embodiments, the payload nucleic acid sequence has 98% identity to one or more of the nucleic acid sequences listed in Tables 3-16, or variants or fragments thereof.

In some embodiments, the payload nucleic acid sequence has 99% identity to one or more of the nucleic acid sequences listed in Tables 3-16, or variants or fragments thereof.

In some embodiments, the payload nucleic acid sequence has 100% identity to one or more of the nucleic acid sequences listed in Tables 3-16, or variants or fragments thereof.

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Tables 3-16, or variants or fragments thereof. As another non-limiting example, the antibody may be one or more of the heavy chain sequences listed in Tables 3-16. As a non-limiting example, the antibody may be one or more of the light chain sequences listed in Tables 3-16, or variants or fragments thereof.

In some embodiments, the payload region of the AAV particle comprises a nucleic acid sequence encoding a polypeptide comprising a heavy chain and a light chain sequence listed in Tables 3-16, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Tables 3-16, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5 to 3′ direction, an antibody light chain sequence, a linker and a heavy chain sequence. In another embodiment, the linker is not used.

In some embodiments, the payload region comprises a nucleic acid sequence encoding, in the 5 to 3′ direction, an antibody light chain sequence from Tables 3-16, one or more linkers from Table 2 and a heavy chain sequence from Tables 3-16.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Tables 3-16, one or more linkers from Table 2, and alight chain sequence from Tables 3-16.

In some embodiments, the payload region comprises a nucleic acid sequence encoding a single heavy chain. As a non-limiting example, the heavy chain is an amino acid sequence or fragment thereof described in Tables 3-16.

Shown in Tables 3-16 are a listing of antibodies and their polynucleotides and/or polypeptides sequences. These sequences may be encoded by or included in the AAV particles of the present disclosure. Variants or fragments of the antibody sequences described in Tables 3-16 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Tables 3-16. CDRs of the antibodies encoded by the viral genomes of the present disclosure may be 50%, 60%, 70%, 80%, 90%, 95% identical to CDRs listed in or incorporated in the sequences of Tables 3-16. Methods of determining CDRs are well known in the art and are described herein. Payload regions may encode antibody variants with one or more heavy chain variable domain (V_{H}) or light chain variable domain (V_{L}) derived from the antibody sequences in Tables 3-16. In some cases, such variants may include bispecific antibodies. Bispecific antibodies encoded by payload regions may comprise variable domain pairs from two different antibodies.

In some embodiments, the AAV particles may comprise a heavy and alight chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any muscle disease-associated antibodies, not limited to those described in Tables 3-16, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the muscle disease-associated antibodies as described in International Publication Number WO2017054086, WO2017062016, WO2017070476, WO2017075119, WO2017075325, WO2017085035, WO2017095487, WO2017100193, WO2017100467, WO2017106236, WO2017106326, WO2017106578, WO2017106684, WO2017106932, WO2017110981, WO2017120344, WO2017132555, WO2017156488, WO2017156500, WO2017180536, WO2017180976, WO2017181011, WO2017181015, WO2017181031, WO2017181039, WO2017189805, WO2017189959, WO2017189963, WO2017189964, WO2017199250, WO2017208211, WO2017217128, WO2017217525, WO2017218824, WO2018009903, WO2018022608, WO2018027329, WO2018030777, WO2018035119, WO2018039506, WO2018049237, WO2018053029, WO2018053434, WO2018067701, WO2018075960 WO2018081282, WO2018085842, WO2018089532, WO2018098480, WO2018119246, WO2018127791, WO2018128779, WO2018129395, WO2018132423, WO2018146199, WO2018148585, WO2018160896, WO2018166495, WO2018167322, WO2018169948, WO2018170408, WO2018175790, WO2018183219, WO2018183376, WO2018185110, WO2018191707, WO2018195418, WO2018213204, WO2018218049, WO2018221521, WO2018226578, WO2018232088, WO2018232366, WO2018237192, WO2019003159, WO2019005503, WO2019009419, WO2019012336, WO2019015673, WO2019023661, WO2019024911, WO2019025908, WO2019028456, WO2019031938, WO2019032898, WO2019042153, WO2019042889, WO2019046338, WO2019046600, WO2019054819, WO2019057992, WO2019060619, WO2019067293, WO2019067815, WO2019073507, WO2019078916, WO2019079809, WO2019089592, WO2019090069, WO2019090074, WO2019090076, WO2019090078, WO2019090081, WO2019090082, WO2019090085, WO2019090088, WO2019090090, WO2019090263, WO2019092505, WO2019092507, WO2019094578, WO2019094700, WO2019020734, WO2018003983, and WO2017170090, the contents of each of which are herein incorporated by reference in their entirety.

In some embodiments, the payload region of the viral particle comprises one or more nucleic acid sequences encoding one or more of the muscle disease payload antibody polypeptides listed in Table 6 of U.S. provisional patent application 62/844,433 (MUS1-MUS485; SEQ ID NO: 5647-6131), the contents of which are herein incorporated by reference in their entirety. A non-exhaustive listing of muscle diseases includes Multiple System Atrophy (MSA), Amyotrophic Lateral Sclerosis (ALS) and Duchenne Muscular Dystrophy (DMD).

### Antibodies for the Treatment of Endocrine and Metabolic Diseases

In some embodiments, the payload region of the AAV particle comprises one or more nucleic acid sequences encoding endocrine and metabolic disease-associated antibodies, variants or fragments thereof.

In some embodiments, the payload region of the AAV particle comprises one or more nucleic acid sequences encoding one or more of the payload antibody polypeptides listed in Tables 3-16, or variants or fragments thereof.

In some embodiments, the payload region of the AAV particle comprises one or more nucleic acid sequences listed in Tables 3-16, or variants or fragments thereof.

In some embodiments, the payload region of the AAV particle comprises a nucleic acid sequence encoding a payload antibody with at least 50% identity to one or more payload antibody polypeptides listed in Tables 3-16. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%,98, 99%, or 100% identity to one or more of the payload antibody polypeptides listed in Tables 3-16, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Tables 3-16, or variants or fragments thereof.

In some embodiments, the variable region sequence(s) of the encoded antibody polypeptide may have 50%, 51%, 52%, 53%, 54%, 55%, 56%, 57%, 58%, 59%,60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%,84%, 85%, 86%, 87%, 88%, 89%, 90%, 91%, 92%, 93%, 94%, 95%, 96%, 97%, 98%, 99%, or 100% identity to one or more of the payload antibody polypeptides listed in Tables 3-16, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Tables 3-16, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%, 78%, 79%, 80%, 81%, 82%, 83%, 84%, 85%%,6, 87%,88%,89%, 90%,91%,92%, 93%, 94%,95%, 96%,97%, 98%,99%, or 100% identity to one or more of the payload light chain antibody polypeptides listed in Tables 3-16, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%,63%,64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Tables 3-16, or variants or fragments thereof.

In some embodiments, the payload antibody has 90% identity to one or more of the antibody polypeptides listed in Tables 3-16, or variants or fragments thereof.

In some embodiments, the payload antibody has 91% identity to one or more of the antibody polypeptides listed in Tables 3-16, or variants or fragments thereof.

In some embodiments, the payload antibody has 92% identity to one or more of the antibody polypeptides listed in Tables 3-16, or variants or fragments thereof.

In some embodiments, the payload antibody has 93% identity to one or more of the antibody polypeptides listed in Tables 3-16, or variants or fragments thereof.

In some embodiments, the payload antibody has 94% identity to one or more of the antibody polypeptides listed in Tables 3-16, or variants or fragments thereof.

In some embodiments, the payload antibody has 95% identity to one or more of the antibody polypeptides listed in Tables 3-16, or variants or fragments thereof.

In some embodiments, the payload antibody has 96% identity to one or more of the antibody polypeptides listed in Tables 3-16, or variants or fragments thereof.

In some embodiments, the payload antibody has 97% identity to one or more of the antibody polypeptides listed in Tables 3-16, or variants or fragments thereof.

In some embodiments, the payload antibody has 98% identity to one or more of the antibody polypeptides listed in Tables 3-16, or variants or fragments thereof.

In some embodiments, the payload antibody has 99% identity to one or more of the antibody polypeptides listed in Tables 3-16, or variants or fragments thereof.

In some embodiments, the payload antibody has 100% identity to one or more of the antibody polypeptides listed in Tables 3-16, or variants or fragments thereof.

In some embodiments, the payload region of the AAV particle comprises a nucleic acid sequence with at least 50% identity to one or more nucleic acid sequences listed in Tables 3-16, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Tables 3-16, or variants or fragments thereof.

In some embodiments, the payload nucleic acid sequence has 90% identity to one or more of the nucleic acid sequences listed in Tables 3-16, or variants or fragments thereof.

In some embodiments, the payload nucleic acid sequence has 91% identity to one or more of the nucleic acid sequences listed in Tables 3-16, or variants or fragments thereof.

In some embodiments, the payload nucleic acid sequence has 92% identity to one or more of the nucleic acid sequences listed in Tables 3-16, or variants or fragments thereof.

In some embodiments, the payload nucleic acid sequence has 93% identity to one or more of the nucleic acid sequences listed in Tables 3-16, or variants or fragments thereof.

In some embodiments, the payload nucleic acid sequence has 94% identity to one or more of the nucleic acid sequences listed in Tables 3-16, or variants or fragments thereof.

In some embodiments, the payload nucleic acid sequence has 95% identity to one or more of the nucleic acid sequences listed in Tables 3-16, or variants or fragments thereof.

In some embodiments, the payload nucleic acid sequence has 96% identity to one or more of the nucleic acid sequences listed in Tables 3-16, or variants or fragments thereof.

In some embodiments, the payload nucleic acid sequence has 97% identity to one or more of the nucleic acid sequences listed in Tables 3-16, or variants or fragments thereof.

In some embodiments, the payload nucleic acid sequence has 98% identity to one or more of the nucleic acid sequences listed in Tables 3-16, or variants or fragments thereof.

In some embodiments, the payload nucleic acid sequence has 99% identity to one or more of the nucleic acid sequences listed in Tables 3-16, or variants or fragments thereof.

In some embodiments, the payload nucleic acid sequence has 100% identity to one or more of the nucleic acid sequences listed in Tables 3-16, or variants or fragments thereof.

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Tables 3-16, or variants or fragments thereof. As another non-limiting example, the antibody may be one or more of the heavy chain sequences listed in Tables 3-16. As a non-limiting example, the antibody may be one or more of the light chain sequences listed in Tables 3-16, or variants or fragments thereof.

In some embodiments, the payload region of the AAV particle comprises a nucleic acid sequence encoding a polypeptide comprising a heavy chain and alight chain sequence listed in Tables 3-16, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Tables 3-16, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Tables 3-16, one or more linkers from Table 2 and a heavy chain sequence from Tables 3-16.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Tables 3.16, one or more linkers from Table 2, and alight chain sequence from Tables 3-16.

In some embodiments, the payload region comprises a nucleic acid sequence encoding a single heavy chain. As a non-limiting example, the heavy chain is an amino acid sequence or fragment thereof described in Tables 3-16.

Shown in Tables 3-16 are a listing of antibodies and their polynucleotides and/or polypeptides sequences. These sequences may be encoded by or included in the AAV particles of the present disclosure. Variants or fragments of the antibody sequences described in Tables 3-16 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Tables 3-16. CDRs of the antibodies encoded by the viral genomes of the present disclosure may be 50%, 60%, 70%, 80%, 90%, 95% identical to CDRs listed in or incorporated in the sequences of Tables 3-16. Methods of determining CDRs are well known in the art and are described herein. Payload regions may encode antibody variants with one or more heavy chain variable domain (V_{H}) or light chain variable domain (V_{L}) derived from the antibody sequences in Tables 3-16. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any endocrine and metabolic disease-associated antibodies, not limited to those described in Tables 3-16, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the endocrine and metabolic system disease-associated antibodies as described in International Publication Number WO2017046676, WO2017049139, WO2017049149, WO2017049452, WO2017053556, WO2017053748, WO2017054086, WO2017055613, WO2017055908, WO2017058944, WO2017059196, WO2017059243, WO2017059289, WO2017062016, WO2017062456, WO2017062619, WO2017062693, WO2017062748, WO2017062888, WO2017064716, WO2017065493, WO2017070475, WO2017070527, WO2017070649, WO2017073619, WO2017075052, WO2017075119, WO2017075252, WO2017075432, WO2017075540, WO2017077085, WO2017079112, WO2017079115, WO2017079116, WO2017079117, WO2017079121, WO2017079150, WO2017079591, WO2017083296, WO2017083441, WO2017083515, WO2017087547, WO2017087589, WO2017091683, WO2017093947, WO2017095744, WO2017096163, WO2017096276, WO2017102789, WO2017103895, WO2017106129, WO2017106352, WO2017106656, WO2017107914, WO2017109496, WO2017112741, WO2017112762, WO2017112775, WO2017112803, WO2017112944, WO2017112954, WO2017112955, WO2017112956, WO2017117384, WO2017117430, WO2017120344, WO2017121877, WO2017123557, WO2017127764, WO2017132457, WO2017132459, WO2017136313, WO2017137503, WO2017139623, WO2017142832, WO2017143069, WO2017144681, WO2017147293, WO2017147368, WO2017147383, WO2017147742, WO2017152076, WO2017153567, WO2017156058, WO2017158064, WO2017158116, WO2017160622, WO2017173349, WO2017173384, WO2017175018, WO2017176760, WO2017177179, WO2017178569, WO2017180461, WO2017180536, WO2017180864, WO2017180913, WO2017181111, WO2017185177, WO2017185492, WO2017185949, WO2017189279, WO2017189805, WO2017189813, WO2017190001, WO2017198741, WO2017201204, WO2017201325, WO2017203450, WO2017205651, WO2017208211, WO2017210058, WO2017210443, WO2017212442, WO2017214458, WO2017214462, WO2017214547, WO2017214548, WO2017218515, WO2018004283, WO2018005054, WO2018005519, WO2018005682, WO2018013918, WO2018014122, WO2018019897, WO2018020476, WO2018026722, WO2018027204, WO2018031865, WO2018035061, WO2018035084, WO2018035119, WO2018039020, WO2018039107, WO2018039274, WO2018044105, WO2018044640, WO2018044903, WO2018044948, WO2018047894, WO2018049118, WO2018049120, WO2018049124, WO2018053029, WO2018053270, WO2018053468, WO2018057776, WO2018057823, WO2018064436, WO2018071792, WO2018071873, WO2018075304, WO2018075375, WO2018075740, WO2018075820, WO2018081370, WO2018081375, WO2018081437, WO2018083248, WO2018085359, WO2018085533, WO2018086139, WO2018089335, WO2018089532, WO2018089890, WO2018091444, WO2018093841, WO2018094112, WO2018094414, WO2018098168, WO2018098348, WO2018098362, WO2018099539, WO2018099978, WO2018102589, WO2018102597. WO2018102785, WO2018104554, WO2018111890, WO2018113781, WO2018115885, WO2018119118, WO2018119314, WO2018119351, WO2018127787, WO2018140586, WO2018140729, WO2018146189, WO2018146253, WO2018146549, WO2018146612, WO2018148585, WO2018151836, WO2018156494, WO2018160917, WO2018165619, WO2018174408, WO2018175476, WO2018175790, WO2018175833, WO2018177220, WO2018178040, WO2018181866, WO2018183041, WO2018183173, WO2018184558, WO2018185043, WO2018187158, WO2018189220, WO2018191074, WO2018191502, WO2018195302, WO2018204303, WO2018208625, WO2018212656, WO2018213260, WO2018215995, WO2018220040, WO2018222675, WO2018222711, WO2018223051, WO2018225041, WO2018226776, WO2018229193, WO2018236904, WO2018237064, WO2018237157, WO2018237326, WO2018237335, WO2019005503, WO2019005756, WO2019006162, WO2019010314, WO2019014091, WO2019018538, WO2019023148, WO2019023410, WO2019027935, WO2019036855, WO2019037711, WO2019040471, WO2019046856, WO2019046858, WO2019046859, WO2019050326, WO2019053612, WO2019055537, WO2019059411, WO2019060707, WO2019066535, WO2019066536, WO2019067978, WO2019068904, WO2019074973, WO2019089472, WO2019089832, WO2019071206, WO2019057805, WO2018237097, WO2018237095, WO2018225781, WO2018187642, WO2018158247, WO2018144773, WO2018136440, WO2018102654, WO2018093331, WO2018071718, WO2018022505, WO2018022407, WO2017177181, WO2017136195, WO2017112824, WO2017104783, WO2017084026, WO2017074065, WO2017074063, WO2017074061, WO2017066204, WO2017062334, WO2017049011, the contents of each of which are herein incorporated by reference in their entirety.

### Antibodies for the Treatment of Nervous System Diseases

In some embodiments, the payload region of the AAV particle comprises one or more nucleic acid sequences encoding nervous system disease-associated antibodies, variants or fragments thereof.

In some embodiments, the payload region of the AAV particle comprises one or more nucleic acid sequences encoding one or more of the payload antibody polypeptides listed in Table 7, Table 8, Table 9, Table 10, or variants or fragments thereof. As used herein, “antibody polynucleotide” refers to a nucleic acid sequence encoding an antibody polypeptide.

In some embodiments, the payload region of the AAV particle comprises one or more nucleic acid sequences listed in Table 7, Table 8, Table 9, Table 10, or variants or fragments thereof.

In some embodiments, the payload region may encode antibodies associated with misfolded SOD1 monomers and/or dimers as described by Maier et al. 2018 (Sci Transl Med. 2018 Dec. 5; 10(470); the contents of which are herein incorporated by reference in their entirety).

In some embodiments, payloads may encode APP associated antibodies taught in U.S. Pat. No. 8,961,972, the contents of which are herein incorporated by reference in their entirety. Such embodiments may include antibody LY3002813 or variants or fragments thereof. In some embodiments, the payload region encodes antibody heavy chain variable regions such as but not limited to SEQ ID NO: 11, 36 and/or 47 of U.S. Pat. No. 8,961,972. In some embodiments, the payload region encodes antibody heavy chain variable regions such as but not limited to SEQ ID NO: 12, 13, 42, 37, and/or 48 of U.S. Pat. No. 8,961,972. In some embodiments, the payload region encodes antibody heavy chain regions such as but not limited to SEQ ID NO: 14, 38 and/or 49 of US Patent Number U88961972. In some embodiments, the payload region encodes antibody heavy chain variable regions such as but not limited to SEQ ID NO: 15, 16, 44, 39, and/or 50 of U.S. Pat. No. 8,961,972.

In some embodiments, the payload region of the AAV particle comprises a nucleic acid sequence encoding a payload antibody with at least 50% identity to one or more payload antibody polypeptides listed in Table 7, Table 8, Table 9, Table 10. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%,91%,92%, 93%, 94%, 95%, 96%, 97%,98%,99%, or 100% identity to one or more of the payload antibody polypeptides listed in Table 7, Table 8, Table 9, Table 10, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%,63%,64%, 65%, 66%,67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 7, Table 8, Table 9, Table 10, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99%, or 100% identity to one or more of the payload antibody polypeptides listed in Table 7, Table 8, Table 9, Table 10, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 7, Table 8, Table 9, Table 10, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 7, Table 8, Table 9, Table 10, or variants or fragments thereof.

In some embodiments, the CDR region of the encoded antibody polypeptide may have 50%, 51%,52%, 53%,54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%,81%,82%, 83%,84%,85%, 86%, 87%, 88%, 89%, 90%,91%, 92%, 93%,94%,95%, 96%,97%,98%, 99%, or 100% identity to the CDRs of one or more of the payload antibody polypeptides listed in Table 7, Table 8, Table 9, Table 10, or variants or fragments thereof.

In some embodiments, the payload antibody has 90% identity to one or more of the antibody polypeptides listed in Table 7, Table 8, Table 9, Table 10, or variants or fragments thereof.

In some embodiments, the payload antibody has 91% identity to one or more of the antibody polypeptides listed in Table 7, Table 8, Table 9, Table 10, or variants or fragments thereof.

In some embodiments, the payload antibody has 92% identity to one or more of the antibody polypeptides listed in Table 7, Table 8, Table 9, Table 10, or variants or fragments thereof.

In some embodiments, the payload antibody has 93% identity to one or more of the antibody polypeptides listed in Table 7, Table 8, Table 9, Table 10, or variants or fragments thereof.

In some embodiments, the payload antibody has 94% identity to one or more of the antibody polypeptides listed in Table 7, Table 8, Table 9, Table 10, or variants or fragments thereof.

In some embodiments, the payload antibody has 95% identity to one or more of the antibody polypeptides listed in Table 7, Table 8, Table 9, Table 10, or variants or fragments thereof.

In some embodiments, the payload antibody has 96% identity to one or more of the antibody polypeptides listed in Table 7, Table 8, Table 9, Table 10, or variants or fragments thereof.

In some embodiments, the payload antibody has 97% identity to one or more of the antibody polypeptides listed in Table 7, Table 8, Table 9, Table 10, or variants or fragments thereof.

In some embodiments, the payload antibody has 98% identity to one or more of the antibody polypeptides listed in Table 7, Table 8, Table 9, Table 10, or variants or fragments thereof.

In some embodiments, the payload antibody has 99% identity to one or more of the antibody polypeptides listed in Table 7, Table 8, Table 9, Table 10, or variants or fragments thereof.

In some embodiments, the payload antibody has 100% identity to one or more of the antibody polypeptides listed in Table 7, Table 8, Table 9, Table 10, or variants or fragments thereof.

In some embodiments, the payload region of the AAV particle comprises a nucleic acid sequence with at least 50% identity to one or more nucleic acid sequences listed in Table 7, Table 8, Table 9, Table 10, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%,89%, 90%, 91%, 92%, 93%, 94%, 95%, 96%, 97%, 98%, 99%, or 100% identity to one or more nucleic acid sequences listed in Table 7, Table 8, Table 9, Table 10, or variants or fragments thereof.

In some embodiments, the payload nucleic acid sequence has 90% identity to one or more of the nucleic acid sequences listed in Table 7, Table 8, Table 9, Table 10, or variants or fragments thereof.

In some embodiments, the payload nucleic acid sequence has 91% identity to one or more of the nucleic acid sequences listed in Table 7, Table 8, Table 9, Table 10, or variants or fragments thereof.

In some embodiments, the payload nucleic acid sequence has 92% identity to one or more of the nucleic acid sequences listed in Table 7, Table 8, Table 9, Table 10, or variants or fragments thereof.

In some embodiments, the payload nucleic acid sequence has 93% identity to one or more of the nucleic acid sequences listed in Table 7, Table 8, Table 9, Table 10, or variants or fragments thereof.

In some embodiments, the payload nucleic acid sequence has 94% identity to one or more of the nucleic acid sequences listed in Table 7, Table 8, Table 9, Table 10, or variants or fragments thereof.

In some embodiments, the payload nucleic acid sequence has 95% identity to one or more of the nucleic acid sequences listed in Table 7, Table 8, Table 9, Table 10, or variants or fragments thereof.

In some embodiments, the payload nucleic acid sequence has 96% identity to one or more of the nucleic acid sequences listed in Table 7, Table 8, Table 9, Table 10, or variants or fragments thereof.

In some embodiments, the payload nucleic acid sequence has 97% identity to one or more of the nucleic acid sequences listed in Table 7, Table 8, Table 9, Table 10, or variants or fragments thereof.

In some embodiments, the payload nucleic acid sequence has 98% identity to one or more of the nucleic acid sequences listed in Table 7, Table 8, Table 9, Table 10, or variants or fragments thereof.

In some embodiments, the payload nucleic acid sequence has 99% identity to one or more of the nucleic acid sequences listed in Table 7, Table 8, Table 9, Table 10, or variants or fragments thereof.

In some embodiments, the payload nucleic acid sequence has 100% identity to one or more of the nucleic acid sequences listed in Table 7, Table 8, Table 9, Table 10, or variants or fragments thereof.

In some embodiments, the payload antibody may be variants of any of the antibody polypeptides listed in Table 7, that exclude one or more amino acids designated as “X” or “x” in the described polypeptide sequence, wherein X may represent any amino acid. In some embodiments, the payload nucleic acid sequence may be variants of any of the nucleic acid sequences listed in Table 7, that exclude one or more nucleic acids designated as “n” or “N” in the described nucleic acid sequence, wherein n may represent any nucleic acid.

TABLE 7
Nervous system disease antibodies

| | | Type | | SEQ |
|---|---|---|---|---|
| Ab ID | Component | (PRT/DNA) | Reference | ID NO |
| NEU1 | Fab | PRT | U.S. 10/093,947; SEQ ID NO: 3 | 7575 |
| NEU2 | HC | DNA | U.S. 10/093,947; SEQ ID NO: 1 | 7576 |
| NEU3 | HC | PRT | WO2013130393; SEQ ID NO: 2 | 7577 |
| NEU4 | HC | PRT | U.S. Pat. No. 9,283,271; SEQ ID NO: 70 | 7578 |
| NEU5 | HC | PRT | U.S. Pat. No. 9,283,271; SEQ ID NO: 66 | 7579 |
| NEU6 | HC | PRT | U.S. Pat. No. 9,283,271; SEQ ID NO: 78 | 7580 |
| NEU7 | HC | PRT | U.S. Pat. No. 9,283,271; SEQ ID NO: 60 | 7581 |
| NEU8 | HC | PRT | U.S. Pat. No. 9,283,271; SEQ ID NO: 68 | 7582 |
| NEU9 | HC | PRT | U.S. Pat. No. 9,283,271; SEQ ID NO: 80 | 7583 |
| NEU10 | HC | PRT | U.S. Pat. No. 9,283,271; SEQ ID NO: 82 | 7584 |
| NEU11 | HC | PRT | U.S. Pat. No. 9,283,271; SEQ ID NO: 72 | 7585 |
| NEU12 | HC | PRT | U.S. Pat. No. 9,283,271; SEQ ID NO: 76 | 7586 |
| NEU13 | HC | PRT | U.S. Pat. No. 9,283,271; SEQ ID NO: 74 | 7587 |
| NEU14 | HC | PRT | U.S. Pat. No. 9,283,271; SEQ ID NO: 64 | 7588 |
| NEU15 | HC | PRT | U.S. Pat. No. 9,283,271; SEQ ID NO: 62 | 7589 |
| NEU16 | LC | PRT | U.S. Pat. No. 9,283,271; SEQ ID NO: 65 | 7590 |
| NEU17 | LC | PRT | U.S. Pat. No. 9,283,271; SEQ ID NO: 71 | 7591 |
| NEU18 | LC | PRT | U.S. Pat. No. 9,283,271; SEQ ID NO: 61 | 7592 |
| NEU19 | LC | PRT | U.S. Pat. No. 9,283,271; SEQ ID NO: 69 | 7593 |
| NEU20 | LC | PRT | U.S. Pat. No. 9,283,271; SEQ ID NO: 83 | 7594 |
| NEU21 | LC | PRT | U.S. Pat. No. 9,283,271; SEQ ID NO: 79 | 7595 |
| NEU22 | LC | PRT | U.S. Pat. No. 9,283,271; SEQ ID NO: 67 | 7596 |
| NEU23 | LC | PRT | U.S. Pat. No. 9,283,271; SEQ ID NO: 63 | 7597 |
| NEU24 | LC | PRT | U.S. Pat. No. 9,283,271; SEQ ID NO: 75 | 7598 |
| NEU25 | LC | PRT | U.S. Pat. No. 9,283,271; SEQ ID NO: 77 | 7599 |
| NEU26 | LC | PRT | U.S. Pat. No. 9,283,271; SEQ ID NO: 73 | 7600 |
| NEU27 | LC | PRT | U.S. Pat. No. 9,283,271; SEQ ID NO: 81 | 7601 |
| NEU28 | VH | DNA | US20160168267; SEQ ID NO: 27 | 7602 |
| NEU29 | VH | DNA | US20160168267; SEQ ID NO: 47 | 7603 |
| NEU30 | VH | DNA | U.S. Pat. No. 9,283,271; SEQ ID NO: 47 | 7604 |
| NEU31 | VH | DNA | US20160168267; SEQ ID NO: 31 | 7605 |
| NEU32 | VH | DNA | US20160168267; SEQ ID NO: 39 | 7606 |
| NEU33 | VH | DNA | US20160168267; SEQ ID NO: 7 | 7607 |
| NEU34 | VH | DNA | US20160168267; SEQ ID NO: 11 | 7608 |
| NEU35 | VH | DNA | US20160168267; SEQ ID NO: 15 | 7609 |
| NEU36 | VH | DNA | US20160168267; SEQ ID NO: 43 | 7610 |
| NEU37 | VH | DNA | US20160168267; SEQ ID NO: 35 | 7611 |
| NEU38 | VH | DNA | US20160168267; SEQ ID NO: 19 | 7612 |
| NEU39 | VH | DNA | U.S. Pat. No. 9,283,271; SEQ ID NO: 19 | 7613 |
| NEU40 | VH | DNA | US20160168267; SEQ ID NO: 3 | 7614 |
| NEU41 | VH | DNA | US20160168267; SEQ ID NO: 23 | 7615 |
| NEU42 | VH | DNA | U.S. Pat. No. 9,283,271; SEQ ID NO: 27 | 7616 |
| NEU43 | VH | PRT | U.S. Pat. No. 9,283,271; SEQ ID NO: 12 | 7617 |
| NEU44 | VH | PRT | U.S. Pat. No. 9,283,271; SEQ ID NO: 8 | 7618 |
| NEU45 | VH | PRT | U.S. Pat. No. 9,283,271; SEQ ID NO: 16 | 7619 |
| NEU46 | VH | PRT | U.S. Pat. No. 9,283,271; SEQ ID NO: 36 | 7620 |
| NEU47 | VH | PRT | U.S. Pat. No. 9,283,271; SEQ ID NO: 40 | 7621 |
| NEU48 | VL | DNA | US20160168267; SEQ ID NO: 49 | 7622 |
| NEU49 | VL | DNA | US20160168267; SEQ ID NO: 41 | 7623 |
| NEU50 | VL | DNA | US20160168267; SEQ ID NO: 21 | 7624 |
| NEU51 | VL | DNA | US20160168267; SEQ ID NO: 5 | 7625 |
| NEU52 | VL | DNA | US20160168267; SEQ ID NO: 13 | 7626 |
| NEU53 | VL | DNA | U.S. Pat. No. 9,283,271; SEQ ID NO: 25 | 7627 |
| NEU54 | VL | DNA | US20160168267; SEQ ID NO: 45 | 7628 |
| NEU55 | VL | DNA | US20160168267; SEQ ID NO: 33 | 7629 |
| NEU56 | VL | DNA | US20160168267; SEQ ID NO: 17 | 7630 |
| NEU57 | VL | DNA | US20160168267; SEQ ID NO: 37 | 7631 |
| NEU58 | VL | DNA | U.S. Pat. No. 9,283,271; SEQ ID NO: 9 | 7632 |
| NEU59 | VL | PRT | U.S. Pat. No. 9,283,271; SEQ ID NO: 6 | 7633 |
| NEU60 | VL | PRT | U.S. Pat. No. 9,283,271; SEQ ID NO: 14 | 7634 |
| NEU61 | VL | PRT | U.S. Pat. No. 9,283,271; SEQ ID NO: 42 | 7635 |
| NEU62 | VL | PRT | U.S. Pat. No. 9,283,271; SEQ ID NO: 34 | 7636 |
| NEU63 | VL | PRT | U.S. Pat. No. 9,283,271; SEQ ID NO: 18 | 7637 |
| NEU64 | VL | PRT | U.S. Pat. No. 9,283,271; SEQ ID NO: 10 | 7638 |
| NEU65 | VL | PRT | U.S. Pat. No. 9,283,271; SEQ ID NO: 38 | 7639 |
| NEU66 | FAB | PRT | WO2017055540; SEQ ID NO: 4 | 7640 |
| NEU67 | Full Antibody | PRT | WO2015155694; SEQ ID NO: 61 | 7641 |
| NEU68 | Full Antibody | PRT | WO2015155694; SEQ ID NO: 63 | 7642 |
| NEU69 | HC | PRT | U.S. Pat. No. 8,562,991; SEQ ID NO: 53 | 7643 |
| NEU70 | HC | PRT | U.S. Pat. No. 8,562,991; SEQ ID NO: 55 | 7644 |
| NEU71 | HC | PRT | U.S. Pat. No. 8,562,991; SEQ ID NO: 57 | 7645 |
| NEU72 | HC | PRT | U.S. Pat. No. 8,562,991; SEQ ID NO: 61 | 7646 |
| NEU73 | HC | PRT | U.S. Pat. No. 8,562,991; SEQ ID NO: 63 | 7647 |
| NEU74 | HC | PRT | U.S. Pat. No. 8,562,991; SEQ ID NO: 65 | 7648 |
| NEU75 | HC | PRT | U.S. Pat. No. 8,562,991; SEQ ID NO: 66 | 7649 |
| NEU76 | HC | PRT | U.S. Pat. No. 8,562,991; SEQ ID NO: 68 | 7650 |
| NEU77 | HC | PRT | U.S. Pat. No. 8,562,991; SEQ ID NO: 70 | 7651 |
| NEU78 | HC | PRT | U.S. Pat. No. 8,562,991; SEQ ID NO: 73 | 7652 |
| NEU79 | HC | PRT | U.S. Pat. No. 8,562,991; SEQ ID NO: 74 | 7653 |
| NEU80 | HC | PRT | U.S. Pat. No. 8,562,991; SEQ ID NO: 75 | 7654 |
| NEU81 | HC | PRT | WO2018195457; SEQ ID NO: 1 | 7655 |
| NEU82 | HC | PRT | WO2018195457; SEQ ID NO: 3 | 7656 |
| NEU83 | HC | PRT | WO2018083628; SEQ ID NO: 1 | 7657 |
| NEU84 | HC | PRT | WO2018083628; SEQ ID NO: 31 | 7658 |
| NEU85 | HC | PRT | WO2017062682; SEQ ID NO: 15 | 7659 |
| NEU86 | HC | PRT | WO2017062682; SEQ ID NO: 27 | 7660 |
| NEU87 | LC | PRT | U.S. Pat. No. 8,562,991; SEQ ID NO: 54 | 7661 |
| NEU88 | LC | PRT | U.S. Pat. No. 8,562,991; SEQ ID NO: 56 | 7662 |
| NEU89 | LC | PRT | U.S. Pat. No. 8,562,991; SEQ ID NO: 58 | 7663 |
| NEU90 | LC | PRT | U.S. Pat. No. 8,562,991; SEQ ID NO: 62 | 7664 |
| NEU91 | LC | PRT | U.S. Pat. No. 8,562,991; SEQ ID NO: 64 | 7665 |
| NEU92 | LC | PRT | U.S. Pat. No. 8,562,991; SEQ ID NO: 67 | 7666 |
| NEU93 | LC | PRT | U.S. Pat. No. 8,562,991; SEQ ID NO: 71 | 7667 |
| NEU94 | LC | PRT | U.S. Pat. No. 8,562,991; SEQ ID NO: 72 | 7668 |
| NEU95 | LC | PRT | U.S. Pat. No. 8,562,991; SEQ ID NO: 76 | 7669 |
| NEU96 | LC | PRT | WO2017055540; SEQ ID NO: 1 | 7670 |
| NEU97 | LC | PRT | WO2017055540; SEQ ID NO: 3 | 7671 |
| NEU98 | LC | PRT | WO2017055540; SEQ ID NO: 14 | 7672 |
| NEU99 | LC | PRT | WO2018195457; SEQ ID NO: 2 | 7673 |
| NEU100 | LC | PRT | WO2018195457; SEQ ID NO: 4 | 7674 |
| NEU101 | LC | PRT | WO2018083628; SEQ ID NO: 12 | 7675 |
| NEU102 | VH | PRT | WO2017210278; SEQ ID NO: 8 | 7676 |
| NEU103 | VH | PRT | WO2017210278; SEQ ID NO: 16 | 7677 |
| NEU104 | VH | PRT | U.S. Pat. No. 8,562,991; SEQ ID NO: 19 | 7678 |
| NEU105 | VH | PRT | U.S. Pat. No. 8,562,991; SEQ ID NO: 20 | 7679 |
| NEU106 | VH | PRT | U.S. Pat. No. 8,562,991; SEQ ID NO: 21 | 7680 |
| NEU107 | VH | PRT | U.S. Pat. No. 8,562,991; SEQ ID NO: 25 | 7681 |
| NEU108 | VH | PRT | U.S. Pat. No. 8,562,991; SEQ ID NO: 26 | 7682 |
| NEU109 | VH | PRT | U.S. Pat. No. 8,562,991; SEQ ID NO: 27 | 7683 |
| NEU110 | VH | PRT | WO2015155694; SEQ ID NO: 64 | 7684 |
| NEU111 | VH | PRT | WO2017055540; SEQ ID NO: 20 | 7685 |
| NEU112 | VH | PRT | WO2018083628; SEQ ID NO: 2 | 7686 |
| NEU113 | VL | PRT | WO2017210278; SEQ ID NO: 9 | 7687 |
| NEU114 | VL | PRT | WO2017210278; SEQ ID NO: 10 | 7688 |
| NEU115 | VL | PRT | WO2017210278; SEQ ID NO: 15 | 7689 |
| NEU116 | VL | PRT | U.S. Pat. No. 8,562,991; SEQ ID NO: 22 | 7690 |
| NEU117 | VL | PRT | U.S. Pat. No. 8,562,991; SEQ ID NO: 23 | 7691 |
| NEU118 | VL | PRT | U.S. Pat. No. 8,562,991; SEQ ID NO: 24 | 7692 |
| NEU119 | VL | PRT | U.S. Pat. No. 8,562,991; SEQ ID NO: 28 | 7693 |
| NEU120 | VL | PRT | U.S. Pat. No. 8,562,991; SEQ ID NO: 29 | 7694 |
| NEU121 | VL | PRT | U.S. Pat. No. 8,562,991; SEQ ID NO: 30 | 7695 |
| NEU122 | VL | PRT | WO2018083628; SEQ ID NO: 13 | 7696 |

In some embodiments, the payload region of the AAV particle may include a nucleic acid sequence encoding a polypeptide which is may be an antibody, an antibody-based composition, or a fragment thereof, which when expressed, results in the silencing, suppression, and/or reduction of any one of mutant, variant and/or wild type APP (amyloid beta precursor protein) gene protein products. In some embodiments, the payload region of the AAV particle may include one or more nucleic acid sequences encoding APP associated antibodies, variants or fragments thereof.

In some embodiments, payloads may encode APP associated antibodies taught in U.S. Pat. No. 8,961,972, the contents of which are herein incorporated by reference in their entirety. Such embodiments may include antibody LY3002813 or variants or fragments thereof. In some embodiments, the payload region encodes antibody heavy chain variable regions such as but not limited to SEQ ID NO: 11, 36 and/or 47 of US Patent Number US8961972. In some embodiments, the payload region encodes antibody heavy chain variable regions such as but not limited to SEQ ID NO: 12, 13, 42, 37, and/or 48 of U.S. Pat. No. 8,961,972. In some embodiments, the payload region encodes antibody heavy chain regions such as but not limited to SEQ ID NO: 14, 38 and/or 49 of U.S. Pat. No. 8,961,972. In some embodiments, the payload region encodes antibody heavy chain variable regions such as but not limited to SEQ ID NO: 15, 16, 44, 39, and/or 50 of U.S. Pat. No. 8,961,972.

In some embodiments, payloads may encode beta-secretase 1 (BACE1) associated antibodies taught in U.S. Pat. No. 8,956,614 or variants or fragments thereof; the contents of which are herein incorporated by reference in their entirety.

In some embodiments, payloads may encode APP associated antibodies (or fragments thereof) such as but not limited to PMN310, described in Gibbs et al. 2019 (Sci Rep. 2019; 9: 9870; the contents of which are herein incorporated by reference in their entirety).

In some embodiments, payloads may encode APP associated antibodies (or fragments thereof) such as but not limited to AF1, described in Julian et al. (Journal of Biological Chemistry, 2019 294, 8438-8451; the contents of which are herein incorporated by reference in their entirety).

In some embodiments, ALS may be associated with cytoplasmic aggregation of TAR DNA binding protein 43 (TDP43). Payload regions described herein may encode TAR DNA binding protein 43 (TDP43) associated antibodies (or fragments). As a non-limiting example, the antibodies described herein may target the RNA recognition motif 1 (RRM1) of TDP43. In some embodiments, the payload regions of the present disclosure may encode the VH7Vk9 antibody described by Pozzi et al. (J Clin Invest. 2019; 129(4):1581-1595; the contents of which are herein incorporated by reference in their entirety). In some embodiments, the payload regions of the present disclosure may encode the VH7Vk9 antibody derived from the sequences described in US Patent Number U.S. Ser. No. 10/202,443; the contents of which are herein incorporated by reference in their entirety). As a non-limiting example, the heavy chain variable region of the VH7Vk9 antibody may be derived from SEQ ID NOs: 1, 2, 4, and/or 5 of U.S. Ser. No. 10/202,443; and light chain variable region derived from SEQ ID Nos 3, and/or 6 of U.S. Ser. No. 10/202,443.

In some embodiments, payloads may encode APP-associated antibodies (or fragments thereof) taught in U.S. Pat. No. 9,944,696, the contents of each of which are herein incorporated by reference in their entirety. Such embodiments may include antibody Donanemab, or fragments thereof.

In some embodiments, payloads may encode APP-associated antibodies (or fragments thereof) taught in US Publication Number US20180333487, the contents of each of which are herein incorporated by reference in their entirety. Such embodiments may include antibody ducanumab, or fragments thereof.

In some embodiments, payloads may encode APP-associated antibodies (or fragments thereof) taught in International Publication Number WO2019040612, the contents of each of which are herein incorporated by reference in their entirety. Such embodiments may include antibody Adacanumab, or fragments thereof.

As a non-limiting example, the APP associated antibody may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, 99%, or 100% identity to one or more of the antibody polypeptides or polynucleotides listed in Table 8, or variants or fragments thereof. As a non-limiting example, the APP associated antibody may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%,95%, 96%,97%,98%, 99%, or 100% identity to one or more of the antibody polypeptides or polynucleotides listed in Table 8, or variants or fragments thereof.

In some embodiments, the payload antibody may be variants of any of the antibody polypeptides listed in Table 8, that exclude one or more amino acids designated as “X” or “x” in the described polypeptide sequence, wherein X may represent any amino acid. In some embodiments, the payload nucleic acid sequence may be variants of any of the nucleic acid sequences listed in Table 8, that exclude one or more nucleic acids designated as “n” or “N” in the described nucleic acid sequence, wherein n may represent any nucleic acid.

TABLE 8
Amyloid antibodies

| | | Type | | SEQ |
|---|---|---|---|---|
| Ab ID | Component | (PRT/DNA) | Reference | ID NO |
| AMYL1 | CDR | DNA | U.S. Pat. No. 8,025,878; SEQ ID NO: 255 | 7697 |
| AMYL2 | CDR | DNA | U.S. Pat. No. 8,025,878; SEQ ID NO: 253 | 7698 |
| AMYL3 | CDR | DNA | U.S. Pat. No. 8,025,878; SEQ ID NO: 167 | 7699 |
| AMYL4 | CDR | DNA | U.S. Pat. No. 8,025,878; SEQ ID NO: 171 | 7700 |
| AMYL5 | CDR | DNA | U.S. Pat. No. 8,025,878; SEQ ID NO: 169 | 7701 |
| AMYL6 | CDR | DNA | U.S. Pat. No. 8,025,878; SEQ ID NO: 257 | 7702 |
| AMYL7 | CDR | PRT | WO2016087944; WO2017211827; SEQ ID NO: 5 | 7703 |
| AMYL8 | CDR | PRT | U.S. 10/047,121; SEQ ID NO: 33 | 7704 |
| AMYL9 | CDR | PRT | WO2017211827; SEQ ID NO: 9 | 7705 |
| AMYL10 | CDR | PRT | WO2015038888; SEQ ID NO: 23 | 7706 |
| AMYL11 | CDR | PRT | U.S. 10/047,121; SEQ ID NO: 20 | 7707 |
| AMYL12 | CDR | PRT | U.S. 10/047,121; SEQ ID NO: 19 | 7708 |
| AMYL13 | CDR | PRT | WO2016087944: WO2017211827; SEQ ID NO: 8 | 7709 |
| AMYL14 | CDR | PRT | U.S. 10/047,121; SEQ ID NO: 38 | 7710 |
| AMYL15 | CDR | PRT | U.S. 10/047,121; SEQ ID NO: 18 | 7711 |
| AMYL16 | CDR | PRT | WO2015038888; SEQ ID NO: 26 | 7712 |
| AMYL17 | CDR | PRT | WO2015038888; SEQ ID NO: 24 | 7713 |
| AMYL18 | CDR | PRT | WO2015038888; SEQ ID NO: 28 | 7714 |
| AMYL19 | CDR | PRT | WO2016087944; WO2017211827; SEQ ID NO: 3 | 7715 |
| AMYL20 | CDR | PRT | U.S. 10/047,121; SEQ ID NO: 35 | 7716 |
| AMYL21 | CDR | PRT | WO2016087944; WO2017211827; SEQ ID NO: 4 | 7717 |
| AMYL22 | CDR | PRT | U.S. 10/047,121; SEQ ID NO: 34 | 7718 |
| AMYL23 | CDR | PRT | U.S. Pat. No. 8,025,878; SEQ ID NO: 7 | 7719 |
| AMYL24 | CDR | PRT | U.S. Pat. No. 8,025,878; SEQ ID NO: 26 | 7720 |
| AMYL25 | CDR | PRT | U.S. Pat. No. 8,025,878; SEQ ID NO: 9 | 7721 |
| AMYL26 | CDR | PRT | U.S. Pat. No. 8,025,878; SEQ ID NO: 32 | 7722 |
| AMYL27 | CDR | PRT | U.S. Pat. No. 8,025,878; SEQ ID NO: 31 | 7723 |
| AMYL28 | CDR | PRT | U.S. Pat. No. 8,025,878; SEQ ID NO: 3 | 7724 |
| AMYL29 | CDR | PRT | U.S. Pat. No. 8,025,878; SEQ ID NO: 6 | 7725 |
| AMYL30 | CDR | PRT | U.S. Pat. No. 8,025,878; SEQ ID NO: 10 | 7726 |
| AMYL31 | CDR | PRT | U.S. Pat. No. 8,025,878; SEQ ID NO: 34 | 7727 |
| AMYL32 | CDR | PRT | U.S. Pat. No. 8,025,878; SEQ ID NO: 11 | 7728 |
| AMYL33 | CDR | PRT | U.S. Pat. No. 8,025,878; SEQ ID NO: 36 | 7729 |
| AMYL34 | CDR | PRT | U.S. Pat. No. 8,025,878; SEQ ID NO: 1 | 7730 |
| AMYL35 | CDR | PRT | U.S. Pat. No. 8,025,878; SEQ ID NO: 29 | 7731 |
| AMYL36 | CDR | PRT | U.S. Pat. No. 8,025,878; SEQ ID NO: 28 | 7732 |
| AMYL37 | CDR | PRT | U.S. Pat. No. 8,025,878; SEQ ID NO: 27 | 7733 |
| AMYL38 | CDR | PRT | U.S. Pat. No. 8,025,878; SEQ ID NO: 8 | 7734 |
| AMYL39 | CDR | PRT | U.S. Pat. No. 8,025,878; SEQ ID NO: 38 | 7735 |
| AMYL40 | CDR | PRT | U.S. Pat. No. 8,025,878; SEQ ID NO: 2 | 7736 |
| AMYL41 | CDR | PRT | U.S. Pat. No. 8,025,878; SEQ ID NO: 12 | 7737 |
| AMYL42 | CDR | PRT | U.S. Pat. No. 8,961,972; SEQ ID NO: 4 | 7738 |
| AMYL43 | CDR | PRT | U.S. Pat. No. 8,961,972; SEQ ID NO: 35 | 7739 |
| AMYL44 | CDR | PRT | U.S. Pat. No. 8,961,972; SEQ ID NO: 52 | 7740 |
| AMYL45 | CDR | PRT | U.S. Pat. No. 8,961,972; SEQ ID NO: 36 | 7741 |
| AMYL46 | CDR | PRT | U.S. Pat. No. 8,961,972; SEQ ID NO: 41 | 7742 |
| AMYL47 | CDR | PRT | U.S. Pat. No. 8,961,972; SEQ ID NO: 9 | 7743 |
| AMYL48 | CDR | PRT | U.S. Pat. No. 8,961,972; SEQ ID NO: 10 | 7744 |
| AMYL49 | CDR | PRT | U.S. Pat. No. 8,961,972; SEQ ID NO: 46 | 7745 |
| AMYL50 | CDR | PRT | U.S. Pat. No. 8,961,972; SEQ ID NO: 54 | 7746 |
| AMYL51 | CDR | PRT | U.S. Pat. No. 8,961,972; SEQ ID NO: 6 | 7747 |
| AMYL52 | CDR | PRT | U.S. Pat. No. 8,961,972; SEQ ID NO: 40 | 7748 |
| AMYL53 | CDR | PRT | U.S. Pat. No. 8,961,972; SEQ ID NO: 7 | 7749 |
| AMYL54 | CDR | PRT | U.S. Pat. No. 8,961,972; SEQ ID NO: 53 | 7750 |
| AMYL55 | CDR | PRT | U.S. Pat. No. 8,961,972; SEQ ID NO: 3 | 7751 |
| AMYL56 | CDR | PRT | U.S. Pat. No. 8,961,972; SEQ ID NO: 45 | 7752 |
| AMYL57 | CDR | PRT | U.S. Pat. No. 8,961,972; SEQ ID NO: 51 | 7753 |
| AMYL58 | CDR | PRT | U.S. Pat. No. 8,961,972; SEQ ID NO: 5 | 7754 |
| AMYL59 | CDR | PRT | U.S. Pat. No. 8,961,972; SEQ ID NO: 8 | 7755 |
| AMYL60 | CDR | PRT | U.S. Pat. No. 9,222,947; SEQ ID NO: 22 | 7756 |
| AMYL61 | CDR | PRT | U.S. Pat. No. 9,222,947; SEQ ID NO: 23 | 7757 |
| AMYL62 | CDR | PRT | U.S. Pat. No. 9,222,947; SEQ ID NO: 27 | 7758 |
| AMYL63 | CDR | PRT | U.S. Pat. No. 9,222,947; SEQ ID NO: 18 | 7759 |
| AMYL64 | CDR | PRT | U.S. Pat. No. 9,222,947; SEQ ID NO: 19 | 7760 |
| AMYL65 | CDR | PRT | U.S. Pat. No. 9,222,947; SEQ ID NO: 16 | 7761 |
| AMYL66 | CDR | PRT | U.S. Pat. No. 9,222,947; SEQ ID NO: 24 | 7762 |
| AMYL67 | CDR | PRT | U.S. Pat. No. 9,222,947; SEQ ID NO: 25 | 7763 |
| AMYL68 | CDR | PRT | U.S. Pat. No. 9,222,947; SEQ ID NO: 17 | 7764 |
| AMYL69 | FAb | PRT | U.S. 10/047,121; SEQ ID NO: 62 | 7765 |
| AMYL70 | FAb | PRT | U.S. 10/047,121; SEQ ID NO: 50 | 7766 |
| AMYL71 | FAb | PRT | U.S. 10/047,121; SEQ ID NO: 54 | 7767 |
| AMYL72 | FAb | PRT | U.S. 10/047,121; SEQ ID NO: 64 | 7768 |
| AMYL73 | FAb | PRT | U.S. 10/047,121; SEQ ID NO: 56 | 7769 |
| AMYL74 | FAb | PRT | U.S. 10/047,121; SEQ ID NO: 52 | 7770 |
| AMYL75 | FAb | PRT | U.S. 10/047,121; SEQ ID NO: 59 | 7771 |
| AMYL76 | FR | DNA | U.S. Pat. No. 8,025,878; SEQ ID NO: 170 | 7772 |
| AMYL77 | FR | DNA | U.S. Pat. No. 8,025,878; SEQ ID NO: 166 | 7773 |
| AMYL78 | FR | DNA | U.S. Pat. No. 8,025,878; SEQ ID NO: 252 | 7774 |
| AMYL79 | FR | DNA | U.S. Pat. No. 8,025,878; SEQ ID NO: 256 | 7775 |
| AMYL80 | FR | DNA | U.S. Pat. No. 8,025,878; SEQ ID NO: 172 | 7776 |
| AMYL81 | FR | DNA | U.S. Pat. No. 8,025,878; SEQ ID NO: 168 | 7777 |
| AMYL82 | FR | DNA | U.S. Pat. No. 8,025,878; SEQ ID NO: 254 | 7778 |
| AMYL83 | FR | DNA | U.S. Pat. No. 8,025,878; SEQ ID NO: 258 | 7779 |
| AMYL84 | FR | DNA | U.S. Pat. No. 8,025,878; SEQ ID NO: 88 | 7780 |
| AMYL85 | FR | PRT | U.S. Pat. No. 8,025,878; SEQ ID NO: 245 | 7781 |
| AMYL86 | FR | PRT | U.S. Pat. No. 8,025,878; SEQ ID NO: 159 | 7782 |
| AMYL87 | FR | PRT | U.S. Pat. No. 8,025,878; SEQ ID NO: 248 | 7783 |
| AMYL88 | FR | PRT | U.S. Pat. No. 8,025,878; SEQ ID NO: 247 | 7784 |
| AMYL89 | FR | PRT | U.S. Pat. No. 8,025,878; SEQ ID NO: 161 | 7785 |
| AMYL90 | FR | PRT | U.S. Pat. No. 8,025,878; SEQ ID NO: 117 | 7786 |
| AMYL91 | FR | PRT | U.S. Pat. No. 8,025,878; SEQ ID NO: 162 | 7787 |
| AMYL92 | Full antibody | DNA | U.S. Pat. No. 8,025,878; SEQ ID NO: 261 | 7788 |
| AMYL93 | Full antibody | DNA | U.S. Pat. No. 8,025,878; SEQ ID NO: 259 | 7789 |
| AMYL94 | Full antibody | DNA | U.S. Pat. No. 8,025,878; SEQ ID NO: 173 | 7790 |
| AMYL95 | Full antibody | DNA | U.S. Pat. No. 8,025,878; SEQ ID NO: 175 | 7791 |
| AMYL96 | Full antibody | PRT | U.S. Pat. No. 8,025,878; SEQ ID NO: 163 | 7792 |
| AMYL97 | Full antibody | PRT | U.S. Pat. No. 8,025,878; SEQ ID NO: 270 | 7793 |
| AMYL98 | Full antibody | PRT | U.S. Pat. No. 8,025,878; SEQ ID NO: 266 | 7794 |
| AMYL99 | Full antibody | PRT | U.S. Pat. No. 8,025,878; SEQ ID NO: 271 | 7795 |
| AMYL100 | Full antibody | PRT | U.S. Pat. No. 8,025,878; SEQ ID NO: 244 | 7796 |
| AMYL101 | Full antibody | PRT | U.S. Pat. No. 8,025,878; SEQ ID NO: 249 | 7797 |
| AMYL102 | Full antibody | PRT | U.S. Pat. No. 8,025,878; SEQ ID NO: 158 | 7798 |
| AMYL103 | Full antibody | PRT | U.S. Pat. No. 8,025,878; SEQ ID NO: 268 | 7799 |
| AMYL104 | Full antibody | PRT | U.S. Pat. No. 8,025,878; SEQ ID NO: 267 | 7800 |
| AMYL105 | Full antibody | PRT | U.S. Pat. No. 8,025,878; SEQ ID NO: 174 | 7801 |
| AMYL106 | Full antibody | PRT | U.S. Pat. No. 8,025,878; SEQ ID NO: 260 | 7802 |
| AMYL107 | HC | DNA | U.S. Pat. No. 8,961,972; SEQ ID NO: 43 | 7803 |
| AMYL108 | HC | DNA | U.S. Pat. No. 8,961,972; SEQ ID NO: 56 | 7804 |
| AMYL109 | HC | PRT | U.S. 10/047,121; SEQ ID NO: 41 | 7805 |
| AMYL110 | HC | PRT | U.S. 10/047,121; SEQ ID NO: 42 | 7806 |
| AMYL111 | HC | PRT | WO2018081642; SEQ ID NO: 92 | 7807 |
| AMYL112 | HC | PRT | U.S. Pat. No. 8,961,972; SEQ ID NO: 24 | 7808 |
| AMYL113 | HC | PRT | U.S. Pat. No. 8,961,972; SEQ ID NO: 23 | 7809 |
| AMYL114 | HC | PRT | U.S. Pat. No. 8,961,972; SEQ ID NO: 50 | 7810 |
| AMYL115 | HC | PRT | U.S. Pat. No. 8,961,972; SEQ ID NO: 34 | 7811 |
| AMYL116 | HC | PRT | U.S. Pat. No. 8,961,972; SEQ ID NO: 39 | 7812 |
| AMYL117 | LC | DNA | U.S. Pat. No. 8,961,972; SEQ ID NO: 55 | 7813 |
| AMYL118 | LC | PRT | U.S. 10/047,121; SEQ ID NO: 44 | 7814 |
| AMYL119 | LC | PRT | U.S. 10/047,121; SEQ ID NO: 43 | 7815 |
| AMYL120 | LC | PRT | U.S. Pat. No. 8,961,972; SEQ ID NO: 49 | 7816 |
| AMYL121 | LC | PRT | U.S. Pat. No. 8,961,972; SEQ ID NO: 33 | 7817 |
| AMYL122 | LC | PRT | U.S. Pat. No. 8,961,972; SEQ ID NO: 38 | 7818 |
| AMYL123 | LC | PRT | U.S. Pat. No. 8,961,972; SEQ ID NO: 22 | 7819 |
| AMYL124 | VH | DNA | U.S. Pat. No. 8,025,878; SEQ ID NO: 113 | 7820 |
| AMYL125 | VH | DNA | U.S. Pat. No. 8,025,878; SEQ ID NO: 151 | 7821 |
| AMYL126 | VH | DNA | U.S. Pat. No. 8,961,972; SEQ ID NO: 19 | 7822 |
| AMYL127 | VH | DNA | U.S. Pat. No. 8,961,972; SEQ ID NO: 18 | 7823 |
| AMYL128 | VH | PRT | U.S. 10/047,121; SEQ ID NO: 25 | 7824 |
| AMYL129 | VH | PRT | U.S. 10/047,121; SEQ ID NO: 30 | 7825 |
| AMYL130 | VH | PRT | U.S. 10/047,121; SEQ ID NO: 29 | 7826 |
| AMYL131 | VH | PRT | U.S. 10/047,121; SEQ ID NO: 27 | 7827 |
| AMYL132 | VH | PRT | WO2017127764; SEQ ID NO: 10 | 7828 |
| AMYL133 | VH | PRT | WO2015038888; SEQ ID NO: 29 | 7829 |
| AMYL134 | VH | PRT | U.S. 10/047,121; SEQ ID NO: 8 | 7830 |
| AMYL135 | VH | PRT | U.S. 10/047,121; SEQ ID NO: 7 | 7831 |
| AMYL136 | VH | PRT | U.S. 10/047,121; SEQ ID NO: 9 | 7832 |
| AMYL137 | VH | PRT | U.S. 10/047,121; SEQ ID NO: 12 | 7833 |
| AMYL138 | VH | PRT | U.S. 10/047,121; SEQ ID NO: 11 | 7834 |
| AMYL139 | VH | PRT | U.S. 10/047,121; SEQ ID NO: 13 | 7835 |
| AMYL140 | VH | PRT | U.S. 10/047,121; SEQ ID NO: 1 | 7836 |
| AMYL141 | VH | PRT | WO2016087944: WO2017211827; SEQ ID NO: 1 | 7837 |
| AMYL142 | VH | PRT | WO2017211827; SEQ ID NO: 10 | 7838 |
| AMYL143 | VH | PRT | U.S. 10/047,121; SEQ ID NO: 6 | 7839 |
| AMYL144 | VH | PRT | U.S. 10/047,121; SEQ ID NO: 10 | 7840 |
| AMYL145 | VH | PRT | U.S. 10/047,121; SEQ ID NO: 3 | 7841 |
| AMYL146 | VH | PRT | U.S. 10/047,121; SEQ ID NO: 4 | 7842 |
| AMYL147 | VH | PRT | U.S. Pat. No. 8,025,878; SEQ ID NO: 132 | 7843 |
| AMYL148 | VH | PRT | U.S. Pat. No. 8,025,878; SEQ ID NO: 148 | 7844 |
| AMYL149 | VH | PRT | U.S. Pat. No. 8,025,878; SEQ ID NO: 115 | 7845 |
| AMYL150 | VH | PRT | U.S. Pat. No. 8,025,878; SEQ ID NO: 48 | 7846 |
| AMYL151 | VH | PRT | U.S. Pat. No. 8,025,878; SEQ ID NO: 51 | 7847 |
| AMYL152 | VH | PRT | U.S. Pat. No. 8,025,878; SEQ ID NO: 50 | 7848 |
| AMYL153 | VH | PRT | U.S. Pat. No. 8,025,878; SEQ ID NO: 54 | 7849 |
| AMYL154 | VH | PRT | U.S. Pat. No. 8,025,878; SEQ ID NO: 53 | 7850 |
| AMYL155 | VH | PRT | U.S. Pat. No. 8,025,878; SEQ ID NO: 120 | 7851 |
| AMYL156 | VH | PRT | U.S. Pat. No. 8,025,878; SEQ ID NO: 121 | 7852 |
| AMYL157 | VH | PRT | U.S. Pat. No. 8,025,878; SEQ ID NO: 126 | 7853 |
| AMYL158 | VH | PRT | U.S. Pat. No. 8,025,878; SEQ ID NO: 150 | 7854 |
| AMYL159 | VH | PRT | U.S. Pat. No. 8,025,878; SEQ ID NO: 130 | 7855 |
| AMYL160 | VH | PRT | U.S. Pat. No. 8,025,878; SEQ ID NO: 129 | 7856 |
| AMYL161 | VH | PRT | U.S. Pat. No. 8,025,878; SEQ ID NO: 127 | 7857 |
| AMYL162 | VH | PRT | U.S. Pat. No. 8,025,878; SEQ ID NO: 52 | 7858 |
| AMYL163 | VH | PRT | U.S. Pat. No. 8,025,878; SEQ ID NO: 55 | 7859 |
| AMYL164 | VH | PRT | U.S. Pat. No. 8,025,878; SEQ ID NO: 56 | 7860 |
| AMYL165 | VH | PRT | U.S. Pat. No. 8,025,878; SEQ ID NO: 149 | 7861 |
| AMYL166 | VH | PRT | U.S. Pat. No. 8,025,878; SEQ ID NO: 147 | 7862 |
| AMYL167 | VH | PRT | U.S. Pat. No. 8,025,878; SEQ ID NO: 123 | 7863 |
| AMYL168 | VH | PRT | U.S. Pat. No. 8,025,878; SEQ ID NO: 114 | 7864 |
| AMYL169 | VH | PRT | U.S. Pat. No. 8,025,878; SEQ ID NO: 138 | 7865 |
| AMYL170 | VH | PRT | U.S. Pat. No. 8,025,878; SEQ ID NO: 152 | 7866 |
| AMYL171 | VH | PRT | U.S. Pat. No. 8,025,878; SEQ ID NO: 122 | 7867 |
| AMYL172 | VH | PRT | U.S. Pat. No. 8,025,878; SEQ ID NO: 135 | 7868 |
| AMYL173 | VH | PRT | U.S. Pat. No. 8,025,878; SEQ ID NO: 125 | 7869 |
| AMYL174 | VH | PRT | U.S. Pat. No. 8,025,878; SEQ ID NO: 128 | 7870 |
| AMYL175 | VH | PRT | U.S. Pat. No. 8,025,878; SEQ ID NO: 124 | 7871 |
| AMYL176 | VH | PRT | U.S. Pat. No. 8,025,878; SEQ ID NO: 133 | 7872 |
| AMYL177 | VH | PRT | U.S. Pat. No. 8,961,972; SEQ ID NO: 21 | 7873 |
| AMYL178 | VH | PRT | U.S. Pat. No. 8,961,972; SEQ ID NO: 48 | 7874 |
| AMYL179 | VH | PRT | U.S. Pat. No. 8,961,972; SEQ ID NO: 12 | 7875 |
| AMYL180 | VH | PRT | U.S. Pat. No. 8,961,972; SEQ ID NO: 15 | 7876 |
| AMYL181 | VH | PRT | U.S. Pat. No. 8,961,972; SEQ ID NO: 32 | 7877 |
| AMYL182 | VH | PRT | U.S. Pat. No. 8,961,972; SEQ ID NO: 13 | 7878 |
| AMYL183 | VH | PRT | U.S. Pat. No. 8,961,972; SEQ ID NO: 37 | 7879 |
| AMYL184 | VH | PRT | U.S. Pat. No. 9,222,947; SEQ ID NO: 12 | 7880 |
| AMYL185 | VH | PRT | U.S. Pat. No. 9,222,947; SEQ ID NO: 14 | 7881 |
| AMYL186 | VL | DNA | U.S. Pat. No. 8,025,878; SEQ ID NO: 104 | 7882 |
| AMYL187 | VL | DNA | U.S. Pat. No. 8,025,878; SEQ ID NO: 237 | 7883 |
| AMYL188 | VL | DNA | U.S. Pat. No. 8,961,972; SEQ ID NO: 17 | 7884 |
| AMYL189 | VL | PRT | U.S. 10/047,121; SEQ ID NO: 2 | 7885 |
| AMYL190 | VL | PRT | U.S. 10/047,121; SEQ ID NO: 15 | 7886 |
| AMYL191 | VL | PRT | U.S. 10/047,121; SEQ ID NO: 14 | 7887 |
| AMYL192 | VL | PRT | U.S. 10/047,121; SEQ ID NO: 16 | 7888 |
| AMYL193 | VL | PRT | U.S. 10/047,121; SEQ ID NO: 5 | 7889 |
| AMYL194 | VL | PRT | WO2016087944; WO2017211827; SEQ ID NO: 2 | 7890 |
| AMYL195 | VL | PRT | WO2017211827; SEQ ID NO: 11 | 7891 |
| AMYL196 | VL | PRT | U.S. 10/047,121; SEQ ID NO: 26 | 7892 |
| AMYL197 | VL | PRT | U.S. 10/047,121; SEQ ID NO: 31 | 7893 |
| AMYL198 | VL | PRT | U.S. 10/047,121; SEQ ID NO: 32 | 7894 |
| AMYL199 | VL | PRT | U.S. 10/047,121; SEQ ID NO: 28 | 7895 |
| AMYL200 | VL | PRT | WO2017127764; SEQ ID NO: 11 | 7896 |
| AMYL201 | VL | PRT | WO2015038888; SEQ ID NO: 30 | 7897 |
| AMYL202 | VL | PRT | U.S. Pat. No. 8,025,878; SEQ ID NO: 42 | 7898 |
| AMYL203 | VL | PRT | U.S. Pat. No. 8,025,878; SEQ ID NO: 39 | 7899 |
| AMYL204 | VL | PRT | U.S. Pat. No. 8,025,878; SEQ ID NO: 209 | 7900 |
| AMYL205 | VL | PRT | U.S. Pat. No. 8,025,878; SEQ ID NO: 230 | 7901 |
| AMYL206 | VL | PRT | U.S. Pat. No. 8,025,878; SEQ ID NO: 206 | 7902 |
| AMYL207 | VL | PRT | U.S. Pat. No. 8,025,878; SEQ ID NO: 202 | 7903 |
| AMYL208 | VL | PRT | U.S. Pat. No. 8,025,878; SEQ ID NO: 201 | 7904 |
| AMYL209 | VL | PRT | U.S. Pat. No. 8,025,878; SEQ ID NO: 205 | 7905 |
| AMYL210 | VL | PRT | U.S. Pat. No. 8,025,878; SEQ ID NO: 236 | 7906 |
| AMYL211 | VL | PRT | U.S. Pat. No. 8,025,878; SEQ ID NO: 227 | 7907 |
| AMYL212 | VL | PRT | U.S. Pat. No. 8,025,878; SEQ ID NO: 197 | 7908 |
| AMYL213 | VL | PRT | U.S. Pat. No. 8,025,878; SEQ ID NO: 203 | 7909 |
| AMYL214 | VL | PRT | U.S. Pat. No. 8,025,878; SEQ ID NO: 44 | 7910 |
| AMYL215 | VL | PRT | U.S. Pat. No. 8,025,878; SEQ ID NO: 195 | 7911 |
| AMYL216 | VL | PRT | U.S. Pat. No. 8,025,878; SEQ ID NO: 207 | 7912 |
| AMYL217 | VL | PRT | U.S. Pat. No. 8,025,878; SEQ ID NO: 223 | 7913 |
| AMYL218 | VL | PRT | U.S. Pat. No. 8,025,878; SEQ ID NO: 43 | 7914 |
| AMYL219 | VL | PRT | U.S. Pat. No. 8,025,878; SEQ ID NO: 46 | 7915 |
| AMYL220 | VL | PRT | U.S. Pat. No. 8,025,878; SEQ ID NO: 45 | 7916 |
| AMYL221 | VL | PRT | U.S. Pat. No. 8,025,878; SEQ ID NO: 116 | 7917 |
| AMYL222 | VL | PRT | U.S. Pat. No. 8,025,878; SEQ ID NO: 193 | 7918 |
| AMYL223 | VL | PRT | U.S. Pat. No. 8,025,878; SEQ ID NO: 235 | 7919 |
| AMYL224 | VL | PRT | U.S. Pat. No. 8,025,878; SEQ ID NO: 188 | 7920 |
| AMYL225 | VL | PRT | U.S. Pat. No. 8,025,878; SEQ ID NO: 225 | 7921 |
| AMYL226 | VL | PRT | U.S. Pat. No. 8,025,878; SEQ ID NO: 189 | 7922 |
| AMYL227 | VL | PRT | U.S. Pat. No. 8,025,878; SEQ ID NO: 190 | 7923 |
| AMYL228 | VL | PRT | U.S. Pat. No. 8,025,878; SEQ ID NO: 185 | 7924 |
| AMYL229 | VL | PRT | U.S. Pat. No. 8,025,878; SEQ ID NO: 186 | 7925 |
| AMYL230 | VL | PRT | U.S. Pat. No. 8,025,878; SEQ ID NO: 191 | 7926 |
| AMYL231 | VL | PRT | U.S. Pat. No. 8,025,878; SEQ ID NO: 47 | 7927 |
| AMYL232 | VL | PRT | U.S. Pat. No. 8,025,878; SEQ ID NO: 41 | 7928 |
| AMYL233 | VL | PRT | U.S. Pat. No. 8,025,878; SEQ ID NO: 192 | 7929 |
| AMYL234 | VL | PRT | U.S. Pat. No. 8,025,878; SEQ ID NO: 181 | 7930 |
| AMYL235 | VL | PRT | U.S. Pat. No. 8,025,878; SEQ ID NO: 105 | 7931 |
| AMYL236 | VL | PRT | U.S. Pat. No. 8,025,878; SEQ ID NO: 238 | 7932 |
| AMYL237 | VL | PRT | U.S. Pat. No. 8,025,878; SEQ ID NO: 179 | 7933 |
| AMYL238 | VL | PRT | U.S. Pat. No. 8,025,878; SEQ ID NO: 182 | 7934 |
| AMYL239 | VL | PRT | U.S. Pat. No. 8,961,972; SEQ ID NO: 47 | 7935 |
| AMYL240 | VL | PRT | U.S. Pat. No. 8,961,972; SEQ ID NO: 11 | 7936 |
| AMYL241 | VL | PRT | U.S. Pat. No. 8,961,972; SEQ ID NO: 14 | 7937 |
| AMYL242 | VL | PRT | U.S. Pat. No. 8,961,972; SEQ ID NO: 31 | 7938 |
| AMYL243 | VL | PRT | U.S. Pat. No. 8,961,972; SEQ ID NO: 20 | 7939 |
| AMYL244 | VL | PRT | U.S. Pat. No. 8,961,972; SEQ ID NO: 16 | 7940 |
| AMYL245 | VL | PRT | U.S. Pat. No. 9,222,947; SEQ ID NO: 15 | 7941 |
| AMYL246 | VL | PRT | U.S. Pat. No. 9,222,947; SEQ ID NO: 13 | 7942 |
| AMYL247 | VR | PRT | U.S. Pat. No. 8,025,878; SEQ ID NO: 20 | 7943 |
| AMYL248 | VR | PRT | U.S. Pat. No. 8,025,878; SEQ ID NO: 24 | 7944 |
| AMYL249 | VR | PRT | U.S. Pat. No. 8,025,878; SEQ ID NO: 23 | 7945 |
| AMYL250 | VR | PRT | U.S. Pat. No. 8,025,878; SEQ ID NO: 21 | 7946 |
| AMYL251 | VR | PRT | U.S. Pat. No. 8,025,878; SEQ ID NO: 19 | 7947 |
| AMYL252 | VR | PRT | U.S. Pat. No. 8,025,878; SEQ ID NO: 22 | 7948 |
| AMYL253 | VR | PRT | U.S. Pat. No. 8,025,878; SEQ ID NO: 17 | 7949 |
| AMYL254 | VR | PRT | U.S. Pat. No. 8,025,878; SEQ ID NO: 14 | 7950 |
| AMYL255 | VR | PRT | U.S. Pat. No. 8,025,878; SEQ ID NO: 13 | 7951 |
| AMYL256 | VR | PRT | U.S. Pat. No. 8,025,878; SEQ ID NO: 18 | 7952 |
| AMYL257 | VR | PRT | U.S. Pat. No. 8,025,878; SEQ ID NO: 15 | 7953 |
| AMYL258 | VR | PRT | U.S. Pat. No. 8,025,878; SEQ ID NO: 16 | 7954 |
| AMYL259 | HC | PRT | WO2017180555; SEQ ID NO: 11 | 7955 |
| AMYL260 | HC | PRT | WO2018031361; SEQ ID NO: 49 | 7956 |
| AMYL261 | HC | PRT | US20180305444; SEQ ID NO: 12 | 7957 |
| AMYL262 | HC | PRT | US20180305444; SEQ ID NO: 20 | 7958 |
| AMYL263 | HC | PRT | WO2017055540; SEQ ID NO: 2 | 7959 |
| AMYL264 | HC | PRT | WO2017055540; SEQ ID NO: 9 | 7960 |
| AMYL265 | HC | PRT | WO2017055540; SEQ ID NO: 10 | 7961 |
| AMYL266 | HC | PRT | WO2017055540; SEQ ID NO: 12 | 7962 |
| AMYL267 | HC | PRT | WO2017055540; SEQ ID NO: 13 | 7963 |
| AMYL268 | HC | PRT | WO2017055540; SEQ ID NO: 15 | 7964 |
| AMYL269 | HC | PRT | WO2017055540; SEQ ID NO: 17 | 7965 |
| AMYL270 | HC | PRT | WO2018005282; SEQ ID NO: 60 | 7966 |
| AMYL271 | HC | PRT | WO2018005282; SEQ ID NO: 64 | 7967 |
| AMYL272 | HC | PRT | U.S. Pat. No. 7,892,544; SEQ ID NO: 17 | 7968 |
| AMYL273 | LC | PRT | WO2017160555; SEQ ID NO: 12 | 7969 |
| AMYL274 | LC | PRT | WO2017160555; SEQ ID NO: 13 | 7970 |
| AMYL275 | LC | PRT | WO2018031361; SEQ ID NO: 50 | 7971 |
| AMYL276 | LC | PRT | US20180305444; SEQ ID NO: 13 | 7972 |
| AMYL277 | LC | PRT | US20180305444; SEQ ID NO: 14 | 7973 |
| AMYL278 | LC | PRT | WO2018005282; SEQ ID NO: 63 | 7974 |
| AMYL279 | VH | PRT | WO2017160555; SEQ ID NO: 8 | 7975 |
| AMYL280 | VH | PRT | WO2018031361; SEQ ID NO: 31 | 7976 |
| AMYL281 | VH | PRT | WO2018031361; SEQ ID NO: 46 | 7977 |
| AMYL282 | VH | PRT | WO2018031361; SEQ ID NO: 48 | 7978 |
| AMYL283 | VH | PRT | WO2018031361; SEQ ID NO: 54 | 7979 |
| AMYL284 | VH | PRT | WO2018031361; SEQ ID NO: 55 | 7980 |
| AMYL285 | VH | PRT | U.S. 10/047,121; SEQ ID NO: 27 | 7981 |
| AMYL286 | VH | PRT | US20180305444; SEQ ID NO: 9 | 7982 |
| AMYL287 | VH | PRT | US20180305444; SEQ ID NO: 19 | 7983 |
| AMYL288 | VH | PRT | U.S. Pat. No. 9,573,994; SEQ ID NO: 13 | 7984 |
| AMYL289 | VH | PRT | U.S. Pat. No. 9,573,994; SEQ ID NO: 14 | 7985 |
| AMYL290 | VH | PRT | U.S. Pat. No. 9,573,994; SEQ ID NO: 15 | 7986 |
| AMYL291 | VH | PRT | U.S. Pat. No. 9,573,994; SEQ ID NO: 16 | 7987 |
| AMYL292 | VH | PRT | U.S. Pat. No. 9,573,994; SEQ ID NO: 17 | 7988 |
| AMYL293 | VH | PRT | U.S. Pat. No. 9,573,994; SEQ ID NO: 18 | 7989 |
| AMYL294 | VH | PRT | WO2018005282; SEQ ID NO: 40 | 7990 |
| AMYL295 | VH | PRT | WO2018005282; SEQ ID NO: 42 | 7991 |
| AMYL296 | VH | PRT | WO2018005282; SEQ ID NO: 44 | 7992 |
| AMYL297 | VH | PRT | WO2018005282; SEQ ID NO: 46 | 7993 |
| AMYL298 | VH | PRT | WO2018005282; SEQ ID NO: 48 | 7994 |
| AMYL299 | VH | PRT | WO2018005282; SEQ ID NO: 50 | 7995 |
| AMYL300 | VH | PRT | WO2018005282; SEQ ID NO: 52 | 7996 |
| AMYL301 | VH | PRT | WO2018005282; SEQ ID NO: 54 | 7997 |
| AMYL302 | VH | PRT | U.S. Pat. No. 7,892,544; SEQ ID NO: 16 | 7998 |
| AMYL303 | VH | PRT | U.S. Pat. No. 7,892,544; SEQ ID NO: 61 | 7999 |
| AMYL304 | VL | PRT | WO2017180555; SEQ ID NO: 9 | 8000 |
| AMYL305 | VL | PRT | WO2017160555; SEQ ID NO: 10 | 8001 |
| AMYL306 | VL | PRT | WO2018031361; SEQ ID NO: 45 | 8002 |
| AMYL307 | VL | PRT | WO2018031361; SEQ ID NO: 47 | 8003 |
| AMYL308 | VL | PRT | WO2018031361; SEQ ID NO: 53 | 8004 |
| AMYL309 | VL | PRT | US20180305444; SEQ ID NO: 10 | 8005 |
| AMYL310 | VL | PRT | US20180305444; SEQ ID NO: 11 | 8006 |
| AMYL311 | VL | PRT | U.S. Pat. No. 9,573,994; SEQ ID NO: 8 | 8007 |
| AMYL312 | VL | PRT | U.S. Pat. No. 9,573,994; SEQ ID NO: 9 | 8008 |
| AMYL313 | VL | PRT | U.S. Pat. No. 9,573,994; SEQ ID NO: 10 | 8009 |
| AMYL314 | VL | PRT | U.S. Pat. No. 9,573,994; SEQ ID NO: 11 | 8010 |
| AMYL315 | VL | PRT | U.S. Pat. No. 9,573,994; SEQ ID NO: 12 | 8011 |
| AMYL316 | VL | PRT | U.S. Pat. No. 9,573,994; SEQ ID NO: 19 | 8012 |
| AMYL317 | VL | PRT | U.S. Pat. No. 9,573,994; SEQ ID NO: 20 | 8013 |
| AMYL318 | VL | PRT | U.S. Pat. No. 9,573,994; SEQ ID NO: 21 | 8014 |
| AMYL319 | VL | PRT | U.S. Pat. No. 9,573,994; SEQ ID NO: 22 | 8015 |
| AMYL320 | VL | PRT | WO2017055540; SEQ ID NO: 21 | 8016 |
| AMYL321 | VL | PRT | WO2018005282; SEQ ID NO: 39 | 8017 |
| AMYL322 | VL | PRT | WO2018005282; SEQ ID NO: 41 | 8018 |
| AMYL323 | VL | PRT | WO2018005282; SEQ ID NO: 43 | 8019 |
| AMYL324 | VL | PRT | WO2018005282; SEQ ID NO: 45 | 8020 |
| AMYL325 | VL | PRT | WO2018005282; SEQ ID NO: 47 | 8021 |
| AMYL326 | VL | PRT | WO2018005282; SEQ ID NO: 49 | 8022 |
| AMYL327 | VL | PRT | WO2018005282; SEQ ID NO: 51 | 8023 |
| AMYL328 | VL | PRT | WO2018005282; SEQ ID NO: 53 | 8024 |
| AMYL329 | VL | PRT | U.S. Pat. No. 7,892,544; SEQ ID NO: 59 | 8025 |
| AMYL330 | VL | PRT | U.S. Pat. No. 7,892,544; SEQ ID NO: 75 | 8026 |

In some embodiments, the payload region of the AAV particle may include a nucleic acid sequence encoding a polypeptide which is an antibody, an antibody-based composition, or a fragment thereof, which when expressed, results in the silencing, suppression, and/or reduction of any one of mutant, variant and/or wild type Homo sapiens synuclein alpha (SCA), Homo sapiens synuclein beta (SNCB), or Homo sapiens synuclein gamma (SNCG) gene protein products. In some embodiments, the payload region of the AAV particle may include one or more nucleic acid sequences encoding synuclein associated disease antibodies, variants or fragments thereof. As anon-limiting example, the synuclein associated disease antibody may have 50%, 51%, 52%, 53%, 54%, 55%, 56%, 57%, 58%, 59%, 60%, 61%, 62%, 63%, 64%, 65%, 66%, 67%, 68%, 69%, 79%, 71%, 72%, 73%, 74%, 75%: 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the antibody polypeptides or polynucleotides listed in Table 9, or variants or fragments thereof.

In some embodiments, the payload antibody may be variants of any of the antibody polypeptides listed in Table 9, that exclude one or more amino acids designated as “X” or “x” in the described polypeptide sequence, wherein X may represent any amino acid. In some embodiments, the payload nucleic acid sequence may be variants of any of the nucleic acid sequences listed in Table 9, that exclude one or more nucleic acids designated as “n” or “N” in the described nucleic acid sequence, wherein n may represent any nucleic acid.

TABLE 9
Synuclein antibodies

| | | Type | | SEQ |
|---|---|---|---|---|
| Ab ID | Component | (PRT/DNA) | Reference | ID NO |
| SYN1 | CDR | PRT | U.S. Pat. No. 9,222,947; SEQ ID NO: 21 | 8027 |
| SYN2 | CDR | PRT | US20190062415; SEQ ID NO: 11 | 8028 |
| SYN3 | CDR | PRT | U.S. Pat. No. 9,580,493; SEQ ID NO: 18 | 8029 |
| SYN4 | CDR | PRT | US20190062415; SEQ ID NO: 12 | 8030 |
| SYN5 | CDR | PRT | U.S. Pat. No. 9,580,493; SEQ ID NO: 6 | 8031 |
| SYN6 | CDR | PRT | U.S. Pat. No. 9,580,493; SEQ ID NO: 12 | 8032 |
| SYN7 | CDR | PRT | U.S. Pat. No. 9,580,493; SEQ ID NO: 23 | 8033 |
| SYN8 | CDR | PRT | U.S. Pat. No. 9,580,493; SEQ ID NO: 16 | 8034 |
| SYN9 | CDR | PRT | US20190062415; SEQ ID NO: 10 | 8035 |
| SYN10 | CDR | PRT | U.S. Pat. No. 9,580,493; SEQ ID NO: 25 | 8036 |
| SYN11 | CDR | PRT | U.S. Pat. No. 9,580,493; SEQ ID NO: 13 | 8037 |
| SYN12 | CDR | PRT | U.S. Pat. No. 9,580,493; SEQ ID NO: 7 | 8038 |
| SYN13 | CDR | PRT | U.S. Pat. No. 9,580,493; SEQ ID NO: 11 | 8039 |
| SYN14 | CDR | PRT | U.S. Pat. No. 9,580,493; SEQ ID NO: 17 | 8040 |
| SYN15 | Full antibody | PRT | U.S. Pat. No. 9,580,493; SEQ ID NO: 10 | 8041 |
| SYN16 | VH | DNA | US20150232542; SEQ ID NO: 2 | 8042 |
| SYN17 | VH | DNA | US20150232542; SEQ ID NO: 8 | 8043 |
| SYN18 | VH | DNA | US20150232542; SEQ ID NO: 14 | 8044 |
| SYN19 | VH | DNA | US20190062415; SEQ ID NO: 5 | 8045 |
| SYN20 | VH | DNA | U.S. Pat. No. 9,580,493; SEQ ID NO: 21 | 8046 |
| SYN21 | VH | DNA | U.S. Pat. No. 9,580,493; SEQ ID NO: 19 | 8047 |
| SYN22 | VH | PRT | US20150232542; SEQ ID NO: 14 | 8048 |
| SYN23 | VH | PRT | US20150232542; SEQ ID NO: 2 | 8049 |
| SYN24 | VH | PRT | US20150232542; SEQ ID NO: 8 | 8050 |
| SYN25 | VH | PRT | U.S. Pat. No. 9,580,493; SEQ ID NO: 15 | 8051 |
| SYN26 | VH | PRT | US20190062415; SEQ ID NO: 6 | 8052 |
| SYN27 | VH | PRT | US20190062415; SEQ ID NO: 9 | 8053 |
| SYN28 | VH | PRT | US20190062415; SEQ ID NO: 16 | 8054 |
| SYN29 | VH | PRT | US20190062415; SEQ ID NO: 14 | 8055 |
| SYN30 | VH | PRT | US20190062415; SEQ ID NO: 17 | 8056 |
| SYN31 | VH | PRT | US20190062415; SEQ ID NO: 15 | 8057 |
| SYN32 | VH | PRT | US20190062415; SEQ ID NO: 18 | 8058 |
| SYN33 | VH | PRT | U.S. Pat. No. 9,580,493; SEQ ID NO: 20 | 8059 |
| SYN34 | VH | PRT | US20190062415; SEQ ID NO: 13 | 8060 |
| SYN35 | VL | DNA | US20150232542; SEQ ID NO: 11 | 8061 |
| SYN36 | VL | DNA | US20150232542; SEQ ID NO: 5 | 8062 |
| SYN37 | VL | DNA | US20150232542; SEQ ID NO: 20 | 8063 |
| SYN38 | VL | DNA | US20150232542; SEQ ID NO: 17 | 8064 |
| SYN39 | VL | DNA | US20190062415; SEQ ID NO: 7 | 8065 |
| SYN40 | VL | DNA | U.S. Pat. No. 9,580,493; SEQ ID NO: 27 | 8066 |
| SYN41 | VL | DNA | U.S. Pat. No. 9,580,493; SEQ ID NO: 28 | 8067 |
| SYN42 | VL | PRT | US20150232542; SEQ ID NO: 17 | 8068 |
| SYN43 | VL | PRT | US20150232542; SEQ ID NO: 20 | 8069 |
| SYN44 | VL | PRT | US20150232542; SEQ ID NO: 5 | 8070 |
| SYN45 | VL | PRT | US20150232542; SEQ ID NO: 11 | 8071 |
| SYN46 | VL | PRT | U.S. Pat. No. 9,580,493; SEQ ID NO: 26 | 8072 |
| SYN47 | VL | PRT | U.S. Pat. No. 9,580,493; SEQ ID NO: 22 | 8073 |
| SYN48 | VL | PRT | U.S. Pat. No. 9,580,493; SEQ ID NO: 9 | 8074 |
| SYN49 | VL | PRT | US20190062415; SEQ ID NO: 8 | 8075 |
| SYN50 | VL | PRT | U.S. Pat. No. 9,580,493; SEQ ID NO: 14 | 8076 |

In some embodiments, the payload region of the AAV particle may include a nucleic acid sequence encoding a polypeptide which is an antibody, an antibody-based composition, or a fragment thereof, which when expressed, results in the silencing, suppression, and/or reduction of any one of apolipoprotein E (APOE) allele (e.g., ApoE2, ApoE3 and/or ApoE4) protein products. In some embodiments, the payload region of the AAV particle may include one or more nucleic acid sequences encoding APOE associated disease antibodies, variants or fragments thereof.

In some embodiments, payloads may encode APOE antibodies taught in International Publication Number WO2013168174 or variants or fragments thereof; the contents of which are herein incorporated by reference in their entirety.

As a non-limiting example, the APOE associated disease antibody may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the antibody polypeptides or polynucleotides listed in Table 10, or variants or fragments thereof.

In some embodiments, the payload antibody may be variants of any of the antibody polypeptides listed in Table 10, that exclude one or more amino acids designated as “X” or “x” in the described polypeptide sequence, wherein X may represent any amino acid. In some embodiments, the payload nucleic acid sequence may be variants of any of the nucleic acid sequences listed in Table 10, that exclude one or more nucleic acids designated as “n” or “N” in the described nucleic acid sequence, wherein n may represent any nucleic acid.

TABLE 10
APOE antibodies

| | | Type | | SEQ |
|---|---|---|---|---|
| Ab ID | Component | (PRT/DNA) | Reference | ID NO |
| APOE1 | VH | PRT | WO2018081642; SEQ ID NO: 104 | 8077 |
| APOE2 | VH | PRT | WO2018081642; SEQ ID NO: 77 | 8078 |
| APOE3 | VH | PRT | WO2018081642; SEQ ID NO: 50 | 8079 |
| APOE4 | VH | PRT | WO2018081642; SEQ ID NO: 84 | 8080 |
| APOE5 | VH | PRT | WO2018081642; SEQ ID NO: 85 | 8081 |
| APOE6 | VH | PRT | WO2018081642; SEQ ID NO: 58 | 8082 |
| APOE7 | VH | PRT | WO2018081642; SEQ ID NO: 54 | 8083 |
| APOE8 | VH | PRT | WO2018081642; SEQ ID NO: 62 | 8084 |
| APOE9 | VH | PRT | WO2018081642; SEQ ID NO: 126 | 8085 |
| APOE10 | VH | PRT | WO2018081642; SEQ ID NO: 33 | 8086 |
| APOE11 | VH | PRT | WO2018081642; SEQ ID NO: 66 | 8087 |
| APOE12 | VH | PRT | WO2018081642; SEQ ID NO: 4 | 8088 |
| APOE13 | VH | PRT | WO2018081642; SEQ ID NO: 18 | 8089 |
| APOE14 | VH | PRT | WO2018081642; SEQ ID NO: 116 | 8090 |
| APOE15 | VH | PRT | WO2018081642; SEQ ID NO: 122 | 8091 |
| APOE16 | VH | PRT | WO2018081642; SEQ ID NO: 98 | 8092 |
| APOE17 | VH | PRT | WO2018081642; SEQ ID NO: 118 | 8093 |
| APOE18 | VH | PRT | WO2018081642; SEQ ID NO: 124 | 8094 |
| APOE19 | VH | PRT | WO2018081642; SEQ ID NO: 109 | 8095 |
| APOE20 | VH | PRT | WO2018081642; SEQ ID NO: 32 | 8096 |
| APOE21 | VH | PRT | WO2018081642; SEQ ID NO: 102 | 8097 |
| APOE22 | VH | PRT | WO2018081642; SEQ ID NO: 65 | 8098 |
| APOE23 | VH | PRT | WO2018081642; SEQ ID NO: 90 | 8099 |
| APOE24 | VH | PRT | WO2018081642; SEQ ID NO: 52 | 8100 |
| APOE25 | VH | PRT | WO2018081642; SEQ ID NO: 56 | 8101 |
| APOE26 | VH | PRT | WO2018081642; SEQ ID NO: 69 | 8102 |
| APOE27 | VH | PRT | WO2018081642; SEQ ID NO: 26 | 8103 |
| APOE28 | VH | PRT | WO2018081642; SEQ ID NO: 75 | 8104 |
| APOE29 | VH | PRT | WO2018081642; SEQ ID NO: 80 | 8105 |
| APOE30 | VH | PRT | WO2018081642; SEQ ID NO: 81 | 8106 |
| APOE31 | VH | PRT | WO2018081642; SEQ ID NO: 28 | 8107 |
| APOE32 | VH | PRT | WO2018081642; SEQ ID NO: 34 | 8108 |
| APOE33 | VH | PRT | WO2018081642; SEQ ID NO: 76 | 8109 |
| APOE34 | VH | PRT | WO2018081642; SEQ ID NO: 16 | 8110 |
| APOE35 | VH | PRT | WO2018081642; SEQ ID NO: 6 | 8111 |
| APOE36 | VH | PRT | WO2018081642; SEQ ID NO: 22 | 8112 |
| APOE37 | VH | PRT | WO2018081642; SEQ ID NO: 8 | 8113 |
| APOE38 | VH | PRT | WO2018081642; SEQ ID NO: 2 | 8114 |
| APOE39 | VH | PRT | WO2018081642; SEQ ID NO: 14 | 8115 |
| APOE40 | VH | PRT | WO2018081642; SEQ ID NO: 12 | 8116 |
| APOE41 | VH | PRT | WO2018081642; SEQ ID NO: 20 | 8117 |
| APOE42 | VH | PRT | WO2018081642; SEQ ID NO: 125 | 8118 |
| APOE43 | VH | PRT | WO2018081642; SEQ ID NO: 119 | 8119 |
| APOE44 | VH | PRT | WO2018081642; SEQ ID NO: 111 | 8120 |
| APOE45 | VH | PRT | WO2018081642; SEQ ID NO: 112 | 8121 |
| APOE46 | VH | PRT | WO2018081642; SEQ ID NO: 10 | 8122 |
| APOE47 | VH | PRT | WO2018081642; SEQ ID NO: 103 | 8123 |
| APOE48 | VH | PRT | WO2018081642; SEQ ID NO: 27 | 8124 |
| APOE49 | VH | PRT | WO2018081642; SEQ ID NO: 67 | 8125 |
| APOE50 | VH | PRT | WO2018081642; SEQ ID NO: 120 | 8126 |
| APOE51 | VH | PRT | WO2018081642; SEQ ID NO: 70 | 8127 |
| APOE52 | VH | PRT | WO2018081642; SEQ ID NO: 57 | 8128 |
| APOE53 | VH | PRT | WO2018081642; SEQ ID NO: 53 | 8129 |
| APOE54 | VH | PRT | WO2018081642; SEQ ID NO: 82 | 8130 |
| APOE55 | VH | PRT | WO2018081642; SEQ ID NO: 83 | 8131 |
| APOE56 | VH | PRT | WO2018081642; SEQ ID NO: 61 | 8132 |
| APOE57 | VH | PRT | WO2018081642; SEQ ID NO: 94 | 8133 |
| APOE58 | VH | PRT | WO2018081642; SEQ ID NO: 113 | 8134 |
| APOE59 | VL | PRT | WO2018081642; SEQ ID NO: 115 | 8135 |
| APOE60 | VL | PRT | WO2018081642; SEQ ID NO: 121 | 8136 |
| APOE61 | VL | PRT | WO2018081642; SEQ ID NO: 3 | 8137 |
| APOE62 | VL | PRT | WO2018081642; SEQ ID NO: 17 | 8138 |
| APOE63 | VL | PRT | WO2018081642; SEQ ID NO: 19 | 8139 |
| APOE64 | VL | PRT | WO2018081642; SEQ ID NO: 15 | 8140 |
| APOE65 | VL | PRT | WO2018081642; SEQ ID NO: 11 | 8141 |
| APOE66 | VL | PRT | WO2018081642; SEQ ID NO: 13 | 8142 |
| APOE67 | VL | PRT | WO2018081642; SEQ ID NO: 9 | 8143 |
| APOE68 | VL | PRT | WO2018081642; SEQ ID NO: 1 | 8144 |
| APOE69 | VL | PRT | WO2018081642; SEQ ID NO: 97 | 8145 |
| APOE70 | VL | PRT | WO2018081642; SEQ ID NO: 21 | 8146 |
| APOE71 | VL | PRT | WO2018081642; SEQ ID NO: 96 | 8147 |
| APOE72 | VL | PRT | WO2018081642; SEQ ID NO: 73 | 8148 |
| APOE73 | VL | PRT | WO2018081642; SEQ ID NO: 105 | 8149 |
| APOE74 | VL | PRT | WO2018081642; SEQ ID NO: 106 | 8150 |
| APOE75 | VL | PRT | WO2018081642; SEQ ID NO: 31 | 8151 |
| APOE76 | VL | PRT | WO2018081642; SEQ ID NO: 88 | 8152 |
| APOE77 | VL | PRT | WO2018081642; SEQ ID NO: 64 | 8153 |
| APOE78 | VL | PRT | WO2018081642; SEQ ID NO: 29 | 8154 |
| APOE79 | VL | PRT | WO2018081642; SEQ ID NO: 63 | 8155 |
| APOE80 | VL | PRT | WO2018081642; SEQ ID NO: 86 | 8156 |
| APOE81 | VL | PRT | WO2018081642; SEQ ID NO: 5 | 8157 |
| APOE82 | VL | PRT | WO2018081642; SEQ ID NO: 7 | 8158 |
| APOE83 | VL | PRT | WO2018081642; SEQ ID NO: 123 | 8159 |
| APOE84 | VL | PRT | WO2018081642; SEQ ID NO: 117 | 8160 |
| APOE85 | VL | PRT | WO2018081642; SEQ ID NO: 107 | 8161 |
| APOE86 | VL | PRT | WO2018081642; SEQ ID NO: 108 | 8162 |
| APOE87 | VL | PRT | WO2018081642; SEQ ID NO: 74 | 8163 |
| APOE88 | VL | PRT | WO2018081642; SEQ ID NO: 51 | 8164 |
| APOE89 | VL | PRT | WO2018081642; SEQ ID NO: 55 | 8165 |
| APOE90 | VL | PRT | WO2018081642; SEQ ID NO: 68 | 8166 |
| APOE91 | VL | PRT | WO2018081642; SEQ ID NO: 99 | 8167 |
| APOE92 | VL | PRT | WO2018081642; SEQ ID NO: 23 | 8168 |
| APOE93 | VL | PRT | WO2018081642; SEQ ID NO: 78 | 8169 |
| APOE94 | VL | PRT | WO2018081642; SEQ ID NO: 79 | 8170 |
| APOE95 | VL | PRT | WO2018081642; SEQ ID NO: 101 | 8171 |
| APOE96 | VL | PRT | WO2018081642; SEQ ID NO: 72 | 8172 |
| APOE97 | VL | PRT | WO2018081642; SEQ ID NO: 30 | 8173 |
| APOE98 | VL | PRT | WO2018081642; SEQ ID NO: 49 | 8174 |

In some embodiments, the payload region of the AAV particle comprises a nucleic acid sequence encoding a polypeptide which is an antibody, an antibody-based composition, or a fragment thereof. As a nonlimiting example, the antibody may be one or more of the polypeptides listed in Table 7, Table 8, Table 9, Table 10, or variants or fragments thereof. As another non-limiting example, the antibody may be one or more of the heavy chain sequences listed in Table 7, Table 8, Table 9, Table 10. As a non-limiting example, the antibody may be one or more of the light chain sequences listed in Table 7, Table 8, Table 9, Table 10, or variants or fragments thereof.

In some embodiments, the payload region of the AAV particle comprises a nucleic acid sequence encoding a polypeptide comprising a heavy chain and alight chain sequence listed in Table 7, Table 8, Table 9, Table 10, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Table 7, Table 8, Table 9, Table 10, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 7, Table 8, Table 9, Table 10, one or more linkers from Table 2 and a heavy chain sequence from Table 7, Table 8, Table 9, Table 10.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 7, Table 8, Table 9, Table 10, one or more linkers from Table 2, and alight chain sequence from Table 7, Table 8, Table 9, Table 10.

In some embodiments, the payload region comprises a nucleic acid sequence encoding a single heavy chain. As a non-limiting example, the heavy chain is an amino acid sequence or fragment thereof described in Table 7, Table 8, Table 9, Table 10.

Shown in Table 7, Table 8, Table 9, Table 10 are a listing of antibodies and their polynucleotides and/or polypeptides sequences. These sequences may be encoded by or included in the AAV particles of the present disclosure. Variants or fragments of the antibody sequences described in Table 7, Table 8, Table 9, Table 10 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 7, Table 8, Table 9, Table 10. CDRs of the antibodies encoded by the viral genomes of the present disclosure may be 50%, 60%,70%, 80%, 90%, 95% identical to CDRs listed in or incorporated in the sequences of Table 7, Table 8, Table 9, Table 10. Methods of determining CDRs are well known in the art and are described herein. Payload regions may encode antibody variants with one or more heavy chain variable domain (V_{H}) or light chain variable domain (V_{L}) derived from the antibody sequences in Table 7, Table 8, Table 9, Table 10. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any nervous system disease-associated antibodies, not limited to those described in Table 7, Table 8, Table 9, Table 10, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the nervous system disease-associated antibodies as described in International Publication Number WO2016059622, WO2017046658, WO2017046676, WO2017046774, WO2017046776, WO2017049035, WO2017049139, WO2017049251, WO2017053807, WO2017059380, WO2017062016, WO2017062615, WO2017062619, WO2017062693, WO2017062820, WO2017062952, WO2017062966, WO2017065837, WO2017068186, WO2017070395, WO2017070476, WO2017072196, WO2017072662, WO2017073981, WO2017074013, WO2017074074, WO2017075119, WO2017075432, WO2017075615, WO2017076308, WO2017077382, WO2017083296, WO2017083488, WO2017085035, WO2017086627, WO2017087587, WO2017087588, WO2017087589, WO2017089895, WO2017091487, WO2017091719, WO2017093408, WO2017093410, WO2017093448, WO2017095088, WO2017095486, WO2017095875, WO2017099712, WO2017100193, WO2017102833, WO2017106061, WO2017106129, WO2017106236, WO2017106352, WO2017106656, WO2017109721, WO2017112536, WO2017112877, WO2017112954, WO2017112955, WO2017112956, WO2017118761, WO2017119434, WO2017120222, WO2017120534, WO2017123556, WO2017123978, WO2017124002, WO2017125487, WO2017125830, WO2017125831, WO2017125871, WO2017127664, WO2017127702, WO2017132459, WO2017132555, WO2017132562, WO2017134234, WO2017134301, WO2017134302, WO2017134305, WO2017134306, WO2017135791, WO2017136313, WO2017136350, WO2017136355, WO2017136693, WO2017136820, WO2017137542, WO2017142928, WO2017147293, WO2017147383, WO2017147719, WO2017149513, WO2017152076, WO2017152102, WO2017153402, WO2017153567, WO2017156488, WO2017159287, WO2017160555, WO2017160622, WO2017165683, WO2017165766, WO2017171373, WO2017172733, WO2017172990, WO2017174586, WO2017175018, WO2017176007, WO2017176760, WO2017176835, WO2017176864, WO2017177955, WO2017178288, WO2017178653, WO2017180555, WO2017180813, WO2017180904, WO2017180993, WO2017181031, WO2017181039, WO2017181098, WO2017181119, WO2017182603, WO2017185037, WO2017186928, WO2017189805, WO2017189959, WO2017189963, WO2017189964, WO2017191062, WO2017191559, WO2017191560, WO2017191561, WO2017192538, WO2017192567, WO2017192668, WO2017193059, WO2017193107, WO2017193115, WO2017194646, WO2017194782, WO2017194783, WO2017197265, WO2017197331, WO2017197376, WO2017199250, WO2017201440, WO2017201442, WO2017201731, WO2017202387, WO2017204277, WO2017205101, WO2017205536, WO2017205538, WO2017205875, WO2017208210, WO2017210138, WO2017210278, WO2017212250, WO2017214338, WO2017214706, WO2017218435, WO2017218515, WO2017219025, WO2017219029, WO2017219687, WO2017219690, WO2017220800, WO2017220988, WO2017220990, WO2017223284, WO2018001858, WO2018005682, WO2018005706, WO2018005967, WO2018006005, WO2018006092, WO2018007592, WO2018009624, WO2018009903, WO2018010846, WO2018011073, WO2018011353, WO2018013714, WO2018013918, WO2018015573, WO2018016884, WO2018018031, WO2018022479, WO2018023136, WO2018026533, WO2018026722, WO2018026992, WO2018027329, WO2018031361, WO2018033749, WO2018034977, WO2018035061, WO2018035084, WO2018035210, WO2018039506, WO2018044619, WO2018044948, WO2018045325, WO2018048234, WO2018049237, WO2018050027, WO2018050699, WO2018053405, WO2018053434, WO2018059502, WO2018060351, WO2018062402, WO2018064190, WO2018068282, WO2018068283, WO2018069927, WO2018071796, WO2018071871, WO2018071913, WO2018073648, WO2018075339, WO2018075792, WO2018075813, WO2018075960, WO2018077893, WO2018081282, WO2018081370, WO2018081375, WO2018081642, WO2018081649, WO2018083538, WO2018084836, WO2018085731, WO2018086139, WO2018086585, WO2018086605, WO2018089532, WO2018091444, WO2018091739, WO2018091740, WO2018098365, WO2018098480, WO2018102589, WO2018102594, WO2018102746, WO2018102785, WO2018102787, WO2018102795, WO2018103884, WO2018106776, WO2018106781, WO2018107058, WO2018107134, WO2018109058, WO2018115225, WO2018115231, WO2018115885, WO2018119001, WO2018119171, WO2018119351, WO2018124107, WO2018124121, WO2018126232, WO2018127519, WO2018127586, WO2018127709, WO2018127710, WO2018127711, WO2018127791, WO2018128779, WO2018129331, WO2018129404, WO2018129524, WO2018129533, WO2018129553, WO2018129559, WO2018132423, WO2018134691, WO2018134787, WO2018136163, WO2018139404, WO2018140026, WO2018140725, WO2018140970, WO2018140973, WO2018141730, WO2018141964, WO2018144637, WO2018144999, WO2018146074, WO2018146199, WO2018148223, WO2018148224, WO2018148445, WO2018148447, WO2018149358, WO2018149938, WO2018151836, WO2018152359, WO2018152530, WO2018152687, WO2018153340, WO2018153372, WO2018154392, WO2018154580, WO2018156509, WO2018156740, WO2018157769, WO2018158658, WO2018160539, WO2018160731, WO2018160896, WO2018161092, WO2018164441, WO2018165062, WO2018165362, WO2018166495, WO2018167322, WO2018169948, WO2018170145, WO2018170351, WO2018170359, WO2018174544, WO2018175179, WO2018175790, WO2018175833, WO2018175988, WO2018178040, WO2018178077, WO2018178950, WO2018182266, WO2018182284, WO2018183175, WO2018183216, WO2018183219, WO2018183366, WO2018185247, WO2018185526, WO2018185709, WO2018187350, WO2018187682, WO2018188047, WO2018189225, WO2018189381, WO2018191545, WO2018191707, WO2018191718, WO2018195008, WO2018195418, WO2018197492, WO2018198091, WO2018199176, WO2018200586, WO2018200812, WO2018201096, WO2018202649, WO2018204352, WO2018204546, WO2018204677, WO2018204679, WO2018204757, WO2018204895, WO2018206790, WO2018208670, WO2018208709, WO2018209346, WO2018210898, WO2018212136, WO2018213204, WO2018213316, WO2018213592, WO2018213665, WO2018213679, WO2018213680, WO2018215831, WO2018217918, WO2018218083, WO2018218185, WO2018218219, WO2018218240, WO2018220216, WO2018220225, WO2018220234, WO2018221521, WO2018221892, WO2018222139, WO2018222685, WO2018222949, WO2018223051, WO2018223098, WO2018223101, WO2018223140, WO2018223923, WO2018224609, WO2018224630, WO2018226776, WO2018226833, WO2018229193, WO2018229194, WO2018229195, WO2018229197, WO2018231254, WO2018232188, WO2018232366, WO2018232372, WO2018233813 WO2018234793, WO2018237192, WO2018237287, WO2018237326, WO2018237338, WO2019003165, WO2019005756, WO2019005847, WO2019007509, WO2019008377, WO2019008378, WO2019008379, WO2019009419, WO2019010224, WO2019010566, WO2019011719, WO2019012014, WO2019012015, WO2019014360, WO2019014623, WO2019015673, WO2019015936, WO2019016213, WO2019016247, WO2019016402, WO2019018640, WO2019020774, WO2019023564, WO2019024911, WO2019024933, WO2019028051, WO2019028367, WO2019028456, WO2019032661, WO2019032662, WO2019032663, WO2019032699, WO2019033046, WO2019033114, WO2019035005, WO2019035034, WO2019036419, WO2019036460, WO2019040617, WO2019042282, WO2019046338, WO2019047932, WO2019050326, WO2019050362, WO2019052562, WO2019054819, WO2019055841, WO2019055842, WO2019057102, WO2019057772, WO2019059771, WO2019062871, WO2019067491, WO2019067499, WO2019067805, WO2019067815, WO2019068904, WO2019070680, WO2019073080, WO2019075136, WO2019075168, WO2019075472, WO2019079240, WO2019079249, WO2019079569, WO2019080858, WO2019080941, WO2019081022, WO2019081595, WO2019084057, WO2019084064, WO2019084067, WO2019084249, WO2019084332, WO2019084460, WO2019085804, WO2019086878, WO2019087087, WO2019089848, WO2019089870, WO2019089973, WO2019090003, WO2019090069, WO2019090074, WO2019090076, WO2019090078, WO2019090081, WO2019090082, WO2019090085, WO2019090088, WO2019090090, WO2019090110, WO2019092451, WO2019092452, WO2019092505, WO2019094482, WO2019094576, WO2019094578, WO2019094595, WO2019094608, WO2019094983, WO2016005466, WO2017211827, WO2018081460, WO2019040612, WO2019077500, WO2019064053, WO2019036432, WO2018227063, WO2018207638, WO2018204153, WO2018195457, WO2018194951, WO2018154390, WO2018128454, WO2018123979, WO2018119299, WO2018119288, WO2018104893, WO2018083628, WO2018017370, WO2018005282, WO2017164349, WO2017123517, WO2017111166, WO2017091745, WO2017072090, and WO2017055540, the contents of each of which are herein incorporated by reference in their entirety.

In some embodiments, the AAV particles may have a payload region comprising any of the nervous system disease-associated antibodies as described in US Patent Number U.S. Ser. No. 10/047,121, U.S. Ser. No. 10/112,990, U.S. Pat. Nos. 9,090,709, 9,573,994, 9,585,956, U89605055, U.S. Pat. Nos. 9,676,840, 9,828,435, and 9,944,696, the contents of each of which are herein incorporated by reference in their entirety.

In some embodiments, the AAV particles may have a payload region comprising any of the nervous system disease-associated antibodies as described in US Patent Publication Number US20160000910, US20160009793, US20160168267, US20160244514, US20160272699, US20170137502, US20170198030, US20170306017, US20170369559, US20180305444, US20180327485, US20180333487, US20190031746, US20190038613, US20190046536, US20190112361, US20190112362, US20190112364, and US20190112365, the contents of each of which are herein incorporated by reference in their entirety.

In some embodiments, payloads may encode nervous system disease-associated antibodies (or fragments thereof) taught in U.S. Pat. No. 8,025,878, the contents of which are herein incorporated by reference in their entirety. Such embodiments may include antibody BAN2401 or 158, or fragments thereof. In certain embodiments, the payload region encodes antibody BAN2401 or 158, or fragments thereof selected from SEQ ID NO: 25-38, 104-120,136,148, 154-158,167,169,171, 173-179, 183, 237-244, 253, 255, 257, 259-271, as described in U.S. Pat. No. 8,025,878.

In some embodiments, payloads may encode nervous system disease-associated antibodies (or fragments thereof) taught in U.S. Pat. No. 8,025,878, the contents of which are herein incorporated by reference in their entirety. Such embodiments may include antibody AF062243, or fragments thereof. In certain embodiments, the payload region encodes antibody AF062243, or fragments thereof selected from SEQ ID NO: 159-163,166,168,170, 172, 104-120, 136, 148, 154-158, as described in U.S. Pat. No. 8,025,878.

In some embodiments, payloads may encode nervous system disease-associated antibodies (or fragments thereof) taught in U.S. Pat. No. 8,025,878, the contents of which are herein incorporated by reference in their entirety. Such embodiments may include antibody AB064054, or fragments thereof. In certain embodiments, the payload region encodes antibody AB064054, or fragments thereof selected from SEQ ID NO: 245-248, 252, 254, 256, 258, as described in U.S. Pat. No. 8,025,878.

In some embodiments, payloads may encode nervous system disease-associated antibodies (or fragments thereof) taught in US Publication Number US20150232542, the contents of which are herein incorporated by reference in their entirety. Such embodiments may include antibody 118054, or fragments thereof. In certain embodiments, the payload region encodes antibody 811054, or fragments thereof selected from SEQ ID NO: 2, 5, 8, 11, 14, 17, 20, as described in US20150232542.

In some embodiments, payloads may encode nervous system disease-associated antibodies (or fragments thereof) taught in US Patent Number U.S. Ser. No. 10/093,947, the contents of which are herein incorporated by reference in their entirety. Such embodiments may include antibody NIC9D9 anti-nicotine monoclonal antibody, or fragments thereof. In certain embodiments, the payload region encodes antibody NIC9D9 anti-nicotine monoclonal antibody, or fragments thereof selected from SEQ ID NO: 1-3, as described in U.S. Ser. No. 10/093,947.

In some embodiments, payloads may encode nervous system disease-associated antibodies (or fragments thereof) taught in US Patent Publication Number US20190062415, the contents of which are herein incorporated by reference in their entirety. Such embodiments may include antibody RG7935, or fragments thereof. In certain embodiments, the payload region encodes RG7935, or fragments thereof selected from SEQ ID NO: 5-18, as described in US20190062415.

In some embodiments, payloads may encode nervous system disease-associated antibodies (or fragments thereof) taught in U.S. Pat. No. 8,961,972, the contents of which are herein incorporated by reference in their entirety. Such embodiments may include antibody LY3002813, or fragments thereof. In certain embodiments, the payload region encodes LY3002813, or fragments thereof selected from SEQ ID NO: 3-56, as described in U.S. Pat. No. 8,961,972.

In some embodiments, payloads may encode nervous system disease-associated antibodies (or fragments thereof) taught in US Patent Number U.S. Ser. No. 10/047,121, the contents of which are herein incorporated by reference in their entirety. Such embodiments may include antibody m4C9, 4C9hum, or m10B3, or fragments thereof. In certain embodiments, the payload region encodes m4C9, 4C9hum, or m10B3, or fragments thereof selected from SEQ ID NO: 1-38, as described in U.S. Ser. No. 10/047,121.

In some embodiments, payloads may encode nervous system disease-associated antibodies (or fragments thereof) taught in International Publication Number WO2015038888, the contents of which are herein incorporated by reference in their entirety. Such embodiments may include antibody MABT5102A, or fragments thereof. In certain embodiments, the payload region encodes MABT5102A, or fragments thereof selected from SEQ ID NO: 2, 3, 5, 6, 7, 8, 9, 10, 11, 23, 24, 26, 27, 28, 29, 30, as described in WO2015038888.

In some embodiments, payloads may encode nervous system disease-associated antibodies (or fragments thereof) taught in International Publication Number WO2016087944 and WO201721127, the contents of each of which are herein incorporated by reference in their entirety. Such embodiments may include antibody BIIB037, or fragments thereof. In certain embodiments, the payload region encodes BIIB037, or fragments thereof selected from SEQ ID NO: 1-11, as described in WO2016087944 and WO2017211827.

In some embodiments, payloads may encode nervous system disease-associated antibodies (or fragments thereof) taught in International Publication Number WO2013055922, the contents of which are herein incorporated by reference in their entirety. Such embodiments may include antibody Pepinemab or fragments thereof. In certain embodiments, the payload region encodes antibody Pepinemab or fragments thereof selected from SEQ ID NO: 9, 10, 17, 18 as described in WO2013055922.

Parkinson's Disease and Dementia with Lewy Bodies Antibodies

In some embodiments, the payload region of the viral particle comprises one or more nucleic acid sequences encoding one or more of the Parkinson's Disease and dementia with Lewy Bodies payload antibody polypeptides listed in Table 3 of U.S. provisional patent application 62/844,433 (PDLB1-PDLB437; SEQ ID NO: 3787-4223), the contents of which are herein incorporated by reference in their entirety.

In some embodiments, the payload region of the viral particle comprises one or more nucleic acid sequences encoding one or more of the polypeptides that comprise a portion of filamentous bacteriophage gene 3 protein (g3p) sufficient to bind to and/or disaggregate amyloid described in International Publication No. WO2014193935, the contents of which are herein incorporated by reference in their entirety. As a non-limiting example, the payload region of the viral particle comprises one or more nucleic acid sequences encoding one or more of the polypeptides described in WO2014193935 may be used to treat, prevent and/or reduce the effects of Parkinson's Disease and/or dementia. As another non-limiting example, the payload region of the viral particle comprises one or more nucleic acid sequences encoding one or more of the polypeptides described in WO2014193935 may be used to treat, prevent and/or reduce the effects of Alzheimer's Disease. As another non-limiting example, the payload region of the viral particle comprises one or more nucleic acid sequences encoding one or more of the polypeptides described in WO2014193935 may be used to treat, prevent and/or reduce the effects of Huntington's Disease. As another non-limiting example, the payload region of the viral particle comprises one or more nucleic acid sequences encoding one or more of the polypeptides described in WO2014193935 may be used to treat, prevent and/or reduce the effects of muscle disease such as, but not limited to, Multiple System Atrophy (MSA), Amyotrophic Lateral Sclerosis (ALS) and Duchenne Muscular Dystrophy (DMD).

### Alzheimer's Disease Antibodies

In some embodiments, the payload region of the viral particle comprises one or more nucleic acid sequences encoding one or more of the Alzheimer's Disease payload antibody polypeptides listed in Table 4 of U.S. provisional patent application 62/844,433 (AD1-AD1178; SEQ ID NO: 4224-5401), the contents of which are herein incorporated by reference in their entirety.

### Huntington's Disease Antibodies

In some embodiments, the payload region of the viral particle comprises one or more nucleic acid sequences encoding one or more of the Huntington's Disease payload antibody polypeptides listed in Table 5 of U.S. provisional patent application 62/844,433 (HD1-HD245; SEQ ID NO: 5402-5646), the contents of which are herein incorporated by reference in their entirety.

### Neuropathy Antibodies

In some embodiments, the payload region of the viral particle comprises one or more nucleic acid sequences encoding one or more of the neuropathy payload antibody polypeptides listed in Table 7 of U.S. provisional patent application 62/844,433 (NEURO1-NEURO65; SEQ ID NO:6132-6196), the contents of which are herein incorporated by reference in their entirety.

### Psychiatric Disorder Antibodies

In some embodiments, the payload region of the viral particle comprises one or more nucleic acid sequences encoding one or more of the psychiatric disorder payload antibody polypeptides listed in Table 8 of U.S. provisional patent application 62/844,433 (PSYCH1-PSYCH160; SEQ ID NO: 6197-6356), the contents of which are herein incorporated by reference in their entirety.

### Tau

In some embodiments, the payload region of the viral particle comprises one or more nucleic acid sequences encoding one or more of the payload antibody polypeptides listed in Table 13 of U.S. provisional patent application 62/844,433 (TAU1-TAU1322; SEQ ID NO: 20979-22300), the contents of which are herein incorporated by reference in their entirety.

Payload regions of the viral genomes of the disclosure may encode any anti-tau antibodies, or tau-associated antibodies, not limited to those described in Table 13, including antibodies that are known in the art and/or antibodies that are commercially available as described in of U.S. provisional patent application 62/844,433. This may include fragments of such antibodies or antibodies that have been developed to comprise one or more of such fragments (e.g., variable domains or complementarity determining regions (CDRs)).

In one embodiment, the payload region of the AAV particle comprises one or more nucleic acid sequences encoding one or more of the payload antibody polypeptides listed in Table 11.

In one embodiment, the payload region of the AAV particle comprises one or more nucleic acid sequences listed in Table 11.

In some embodiments, the payload region of the AAV particle comprises a nucleic acid sequence encoding a payload antibody with at least 50% identity to one or more payload antibody polypeptides listed in Table 11, The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 11.

In one embodiment, the full sequence of the encoded antibody polypeptide may have 50%, 51%, 52%, 53%, 54%, 55%, 56%, 57%, 58%, 59%, 60%, 1%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 11.

In one embodiment, the variable region sequence(s) of the encoded antibody polypeptide may have 50%, 51%, 52%, 53%, 54%, 55%, 56%, 57%, 58%, 59%, 60%, 61%,62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 11.

In one embodiment, the heavy chain of the encoded antibody polypeptide may have 50%,51%,52%,53%, 54%,55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 11.

In one embodiment, the light chain of the encoded antibody polypeptide may have 50%,51%,52%,53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%, 78%, 79%, 80%, 81%, 82%, 83%, 84%, 85%,86, 86%, 87%, 88%, 89%, 90%, 91%,92%, 93%, 94%,95%,96%, 97%,98%,99%, or 100% identity to one or more of the payload light chain antibody polypeptides listed in Table 11.

In one embodiment, the CDR region of the encoded antibody polypeptide may have 50%, 51%,52%,53%, 54%,55%, 56%, 57%, 58%, 59%, 60%, 61%, 62%, 63%,64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 11.

In one embodiment, the payload antibody has 90% identity to one or more of the antibody polypeptides listed in Table 11.

In one embodiment, the payload antibody has 91% identity to one or more of the antibody polypeptides listed in Table 11.

In one embodiment, the payload antibody has 92% identity to one or more of the antibody polypeptides listed in Table 11.

In one embodiment, the payload antibody has 93% identity to one or more of the antibody polypeptides listed in Table 11.

In one embodiment, the payload antibody has 94% identity to one or more of the antibody polypeptides listed in Table 11.

In one embodiment, the payload antibody has 95% identity to one or more of the antibody polypeptides listed in Table 11.

In one embodiment, the payload antibody has 96% identity to one or more of the antibody polypeptides listed in Table 11.

In one embodiment, the payload antibody has 97% identity to one or more of the antibody polypeptides listed in Table 11.

In one embodiment, the payload antibody has 98% identity to one or more of the antibody polypeptides listed in Table 11.

In one embodiment, the payload antibody has 99% identity to one or more of the antibody polypeptides listed in Table 11.

In one embodiment, the payload antibody has 100% identity to one or more of the antibody polypeptides listed in Table 11.

In some embodiments, the payload region of the AAV particle comprises a nucleic acid sequence with at least 50% identity to one or more nucleic acid sequences listed in Table 11. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%,93%,94%, 95%,96%,97%, 98%, 99%, or 100% identity to one or more nucleic acid sequences listed in Table 11.

In one embodiment, the payload nucleic acid sequence has 90% identity to one or more of the nucleic acid sequences listed in Table 11.

In one embodiment, the payload nucleic acid sequence has 91% identity to one or more of the nucleic acid sequences listed in Table 11.

In one embodiment, the payload nucleic acid sequence has 92% identity to one or more of the nucleic acid sequences listed in Table 11.

In one embodiment, the payload nucleic acid sequence has 93% identity to one or more of the nucleic acid sequences listed in Table 11.

In one embodiment, the payload nucleic acid sequence has 94% identity to one or more of the nucleic acid sequences listed in Table 11.

In one embodiment, the payload nucleic acid sequence has 95% identity to one or more of the nucleic acid sequences listed in Table 11.

In one embodiment, the payload nucleic acid sequence has 96% identity to one or more of the nucleic acid sequences listed in Table 11.

In one embodiment, the payload nucleic acid sequence has 97% identity to one or more of the nucleic acid sequences listed in Table 11.

In one embodiment, the payload nucleic acid sequence has 98% identity to one or more of the nucleic acid sequences listed in Table 11.

In one embodiment, the payload nucleic acid sequence has 99% identity to one or more of the nucleic acid sequences listed in Table 11.

In one embodiment, the payload nucleic acid sequence has 100% identity to one or more of the nucleic acid sequences listed in Table 11.

TABLE 11
Tats Associated Disease Antibodies

| | | Type | | SEQ |
|---|---|---|---|---|
| Antibody No. | Component | (PRT/DNA) | Reference | ID NO |
| Tau1323 | VH | PRT | WO2017191560; SEQ ID NO: 7 | 13165 |
| Tau1324 | VL | PRT | WO2017191560; SEQ ID NO: 11 | 13166 |
| Tau1325 | VH | PRT | WO2017191560; SEQ ID NO: 15 | 13167 |
| Tau1326 | VH | PRT | WO2017191560; SEQ ID NO: 16 | 13168 |
| Tau1327 | VH | PRT | WO2017191560; SEQ ID NO: 17 | 13169 |
| Tau1328 | VH | PRT | WO2017191560; SEQ ID NO: 18 | 13170 |
| Tau1329 | VH | PRT | WO2017191560; SEQ ID NO: 19 | 13171 |
| Tau1330 | VL | PRT | WO2017191560; SEQ ID NO: 20 | 13172 |
| Tau1331 | VL | PRT | WO2017191560; SEQ ID NO: 21 | 13173 |
| Tau1332 | VL | PRT | WO2017191560; SEQ ID NO: 22 | 13174 |
| Tau1333 | VL | PRT | WO2017191560; SEQ ID NO: 23 | 13175 |
| Tau1334 | VH | PRT | WO2017191560; SEQ ID NO: 46 | 13176 |
| Tau1335 | VH | PRT | WO2017191560; SEQ ID NO: 47 | 13177 |
| Tau1336 | VH | PRT | WO2017191560; SEQ ID NO: 48 | 13178 |
| Tau1337 | VH | PRT | WO2017191560; SEQ ID NO: 49 | 13179 |
| Tau1338 | VH | PRT | WO2017191560; SEQ ID NO: 50 | 13180 |
| Tau1339 | VH | PRT | WO2017191560; SEQ ID NO: 51 | 13181 |
| Tau1340 | VH | PRT | WO2017191560; SEQ ID NO: 52 | 13182 |
| Tau1341 | VH | PRT | WO2017191560; SEQ ID NO: 53 | 13183 |
| Tau1342 | VH | PRT | WO2017191560; SEQ ID NO: 54 | 13184 |
| Tau1343 | VH | PRT | WO2017191560; SEQ ID NO: 55 | 13185 |
| Tau1344 | VH | PRT | WO2017191560; SEQ ID NO: 56 | 13186 |
| Tau1345 | VH | PRT | WO2017191560; SEQ ID NO: 57 | 13187 |
| Tau1346 | VH | PRT | WO2017191560; SEQ ID NO: 66 | 13188 |
| Tau1347 | HC | PRT | WO2017191560; SEQ ID NO: 72 | 13189 |
| Tau1348 | LC | PRT | WO2017191560; SEQ ID NO: 73 | 13190 |
| Tau1349 | VH | PRT | WO2017191561; SEQ ID NO: 7 | 13191 |
| Tau1350 | VL | PRT | WO2017191561; SEQ ID NO: 11 | 13192 |
| Tau1351 | VH | PRT | WO2017191561; SEQ ID NO: 15 | 13193 |
| Tau1352 | VH | PRT | WO2017191561; SEQ ID NO: 16 | 13194 |
| Tau1353 | VH | PRT | WO2017191561; SEQ ID NO: 17 | 13195 |
| Tau1354 | VH | PRT | WO2017191561; SEQ ID NO: 18 | 13196 |
| Tau1355 | VH | PRT | WO2017191561; SEQ ID NO: 19 | 13197 |
| Tau1356 | VH | PRT | WO2017191561; SEQ ID NO: 26 | 13198 |
| Tau1357 | VH | PRT | WO2017191561; SEQ ID NO: 21 | 13199 |
| Tau1358 | VH | PRT | WO2017191561; SEQ ID NO: 22 | 13200 |
| Tau1359 | VH | PRT | WO2017191561; SEQ ID NO: 23 | 13201 |
| Tau1360 | VH | PRT | WO2017191561; SEQ ID NO: 24 | 13202 |
| Tau1361 | VH | PRT | WO2017191561; SEQ ID NO: 25 | 13203 |
| Tau1362 | VH | PRT | WO2017191561; SEQ ID NO: 26 | 13204 |
| Tau1363 | VH | PRT | WO2017191561; SEQ ID NO: 27 | 13205 |
| Tau1364 | VL | PRT | WO2017191561; SEQ ID NO: 28 | 13206 |
| Tau1365 | VL | PRT | WO2017191561; SEQ ID NO: 29 | 13207 |
| Tau1366 | VL | PRT | WO2017191561; SEQ ID NO: 30 | 13208 |
| Tau1367 | HC | PRT | WO2017191561; SEQ ID NO: 54 | 13209 |
| Tau1368 | LC | PRT | WO2017191561; SEQ ID NO: 55 | 13210 |
| Tau1369 | LC | PRT | WO2018031361; SEQ ID NO: 67 | 13211 |
| Tau1370 | HC | PRT | WO2018031361; SEQ ID NO: 68 | 13212 |
| Tau1371 | VL | PRT | WO2018031361; SEQ ID NO: 75 | 13213 |
| Tau1372 | VH | PRT | WO2018031361; SEQ ID NO: 76 | 13214 |
| Tau1373 | VH | PRT | US20170137502; SEQ ID NO: 68 | 13215 |
| Tau1374 | VL | PRT | US20170137502; SEQ ID NO: 69 | 13216 |
| Tau1375 | VH | PRT | US20170137502; SEQ ID NO: 76 | 13217 |
| Tau1376 | VL | PRT | US20170137502; SEQ ID NO: 77 | 13218 |
| Tau1377 | VH | PRT | US20170137502; SEQ ID NO: 88 | 13219 |
| Tau1378 | VL | PRT | US20170137502; SEQ ID NO: 92 | 13220 |
| Tau1379 | VH | PRT | US20170137502; SEQ ID NO: 96 | 13221 |
| Tau1380 | VL | PRT | US20170137502; SEQ ID NO: 97 | 13222 |
| Tau1381 | VH | PRT | US20170137502; SEQ ID NO: 104 | 13223 |
| Tau1382 | VL | PRT | US20170137502; SEQ ID NO: 105 | 13224 |
| Tau1383 | VL | PRT | US20170137502; SEQ ID NO: 116 | 13225 |
| Tau1384 | VL | PRT | US20170137502; SEQ ID NO: 118 | 13226 |
| Tau1385 | VH | PRT | US20190112362; SEQ ID NO: 10 | 13227 |
| Tau1386 | VL | PRT | US20190112362; SEQ ID NO: 11 | 13228 |
| Tau1387 | VL | PRT | US20190112362; SEQ ID NO: 21 | 13229 |
| Tau1388 | VH | PRT | US20190112362; SEQ ID NO: 30 | 13230 |
| Tau1389 | VL | PRT | US20190112362; SEQ ID NO: 31 | 13231 |
| Tau1390 | VH | PRT | US20190112362; SEQ ID NO: 40 | 13232 |
| Tau1391 | VL | PRT | US20190112362; SEQ ID NO: 41 | 13233 |
| Tau1392 | VH | PRT | US20190112362; SEQ ID NO: 50 | 13234 |
| Tau1393 | VL | PRT | US20190112362; SEQ ID NO: 51 | 13235 |
| Tau1394 | VH | PRT | US20190112362; SEQ ID NO: 60 | 13236 |
| Tau1395 | VL | PRT | US20190112362; SEQ ID NO: 61 | 13237 |
| Tau1396 | VH | PRT | US20190112362; SEQ ID NO: 70 | 13238 |
| Tau1397 | VL | PRT | US20190112362; SEQ ID NO: 71 | 13239 |
| Tau1398 | VH | PRT | US20190112362; SEQ ID NO: 80 | 13240 |
| Tau1399 | VL | PRT | US20190112362; SEQ ID NO: 81 | 13241 |
| Tau1400 | VH | PRT | US20190112362; SEQ ID NO: 90 | 13242 |
| Tau1401 | VL | PRT | US20190112362; SEQ ID NO: 91 | 13243 |
| Tau1402 | VH | PRT | US20190112362; SEQ ID NO: 100 | 13244 |
| Tau1403 | VL | PRT | US20190112362; SEQ ID NO: 101 | 13245 |
| Tau1404 | VH | PRT | US20190112362; SEQ ID NO: 120 | 13246 |
| Tau1405 | VL | PRT | US20190112362; SEQ ID NO: 121 | 13247 |
| Tau1406 | VH | PRT | US20190112362; SEQ ID NO: 130 | 13248 |
| Tau1407 | VL | PRT | US20190112362; SEQ ID NO: 131 | 13249 |
| Tau1408 | VH | PRT | US20190112362; SEQ ID NO: 140 | 13250 |
| Tau1409 | VL | PRT | US20190112362; SEQ ID NO: 141 | 13251 |
| Tau1410 | VH | PRT | US20190112362; SEQ ID NO: 150 | 13252 |
| Tau1411 | VL | PRT | US20190112362; SEQ ID NO: 151 | 13253 |
| Tau1412 | VH | PRT | US20190112362; SEQ ID NO: 160 | 13254 |
| Tau1413 | VL | PRT | US20190112362; SEQ ID NO: 161 | 13255 |
| Tau1414 | VH | PRT | US20190112362; SEQ ID NO: 170 | 13256 |
| Tau1415 | VH | PRT | US20190112362; SEQ ID NO: 180 | 13257 |
| Tau1416 | VL | PRT | US20190112362; SEQ ID NO: 181 | 13258 |
| Tau1417 | VH | PRT | US20190112362; SEQ ID NO: 190 | 13259 |
| Tau1418 | VL | PRT | US20190112362; SEQ ID NO: 191 | 13260 |
| Tau1419 | VH | PRT | US20190112362; SEQ ID NO: 200 | 13261 |
| Tau1420 | VL | PRT | US20190112362; SEQ ID NO: 201 | 13262 |
| Tau1421 | VH | PRT | US20190112362; SEQ ID NO: 210 | 13263 |
| Tau1422 | VL | PRT | US20190112362; SEQ ID NO: 211 | 13264 |
| Tau1423 | VH | PRT | US20190112362; SEQ ID NO: 220 | 13265 |
| Tau1424 | VL | PRT | US20190112362; SEQ ID NO: 221 | 13266 |
| Tau1425 | VH | PRT | US20190112362; SEQ ID NO: 230 | 13267 |
| Tau1426 | VL | PRT | US20190112362; SEQ ID NO: 231 | 13268 |
| Tau1427 | VH | PRT | US20190112362; SEQ ID NO: 240 | 13269 |
| Tau1428 | VL | PRT | US20190112362; SEQ ID NO: 241 | 13270 |
| Tau1429 | VH | PRT | US20190112362; SEQ ID NO: 250 | 13271 |
| Tau1430 | VL | PRT | US20190112362; SEQ ID NO: 251 | 13272 |
| Tau1431 | VH | PRT | US20190112362; SEQ ID NO: 260 | 13273 |
| Tau1432 | VL | PRT | US20190112362; SEQ ID NO: 261 | 13274 |
| Tau1433 | VH | PRT | US20190112362; SEQ ID NO: 270 | 13275 |
| Tau1434 | VL | PRT | US20190112362; SEQ ID NO: 271 | 13276 |
| Tau1435 | VH | PRT | US20190112362; SEQ ID NO: 280 | 13277 |
| Tau1436 | VL | PRT | US20190112362; SEQ ID NO: 281 | 13278 |
| Tau1437 | HC | PRT | US20190112362; SEQ ID NO: 288 | 13279 |
| Tau1438 | LC | PRT | US20190112362; SEQ ID NO: 289 | 13280 |
| Tau1439 | VL | PRT | US20190112362; SEQ ID NO: 291 | 13281 |
| Tau1440 | VH | PRT | US20190112362; SEQ ID NO: 300 | 13282 |
| Tau1441 | VL | PRT | US20190112362; SEQ ID NO: 301 | 13283 |
| Tau1442 | VH | PRT | US20190112362; SEQ ID NO: 310 | 13284 |
| Tau1443 | VL | PRT | US20190112362; SEQ ID NO: 311 | 13285 |
| Tau1444 | VH | PRT | US20190112362; SEQ ID NO: 320 | 13286 |
| Tau1445 | VL | PRT | US20190112362; SEQ ID NO: 321 | 13287 |
| Tau1446 | VL | PRT | US20190112362; SEQ ID NO: 331 | 13288 |
| Tau1447 | VL | PRT | US20190112362; SEQ ID NO: 341 | 13289 |
| Tau1448 | HC | PRT | US20190112362; SEQ ID NO: 348 | 13290 |
| Tau1449 | LC | PRT | US20190112362; SEQ ID NO: 349 | 13291 |
| Tau1450 | LC | PRT | US20190112362; SEQ ID NO: 444 | 13292 |
| Tau1451 | HC | PRT | US20190112362; SEQ ID NO: 446 | 13293 |
| Tau1452 | HC | PRT | US20190112362; SEQ ID NO: 447 | 13294 |
| Tau1453 | HC | PRT | US20190112362; SEQ ID NO: 450 | 13295 |
| Tau1454 | HC | PRT | US20190112362; SEQ ID NO: 451 | 13296 |
| Tau1455 | HC | PRT | US20190112362; SEQ ID NO: 453 | 13297 |
| Tau1456 | HC | PRT | US20190112362; SEQ ID NO: 454 | 13298 |
| Tau1457 | HC | PRT | US20190112362; SEQ ID NO: 455 | 13299 |
| Tau1458 | HC | PRT | US20190112362; SEQ ID NO: 456 | 13300 |
| Tau1459 | HC | PRT | US20190112362; SEQ ID NO: 457 | 13301 |
| Tau1460 | HC | PRT | US20190112362; SEQ ID NO: 458 | 13302 |
| Tau1461 | HC | PRT | US20190112362; SEQ ID NO: 459 | 13303 |
| Tau1462 | LC | PRT | US20190112362; SEQ ID NO: 460 | 13304 |
| Tau1463 | LC | PRT | US20190112362; SEQ ID NO: 461 | 13305 |
| Tau1464 | LC | PRT | US20190112362; SEQ ID NO: 462 | 13306 |
| Tau1465 | LC | PRT | US20190112362; SEQ ID NO: 463 | 13307 |
| Tau1466 | LC | PRT | US20190112362; SEQ ID NO: 465 | 13308 |
| Tau1467 | LC | PRT | US20190112362; SEQ ID NO: 466 | 13309 |
| Tau1468 | LC | PRT | US20190112362; SEQ ID NO: 467 | 13310 |
| Tau1469 | VL | PRT | US20190112362; SEQ ID NO: 561 | 13311 |
| Tau1470 | LC | PRT | US20190112362; SEQ ID NO: 569 | 13312 |
| Tau1471 | VL | PRT | US20190112362; SEQ ID NO: 571 | 13313 |
| Tau1472 | LC | PRT | US20190112362; SEQ ID NO: 579 | 13314 |
| Tau1473 | VL | PRT | US20190112362; SEQ ID NO: 581 | 13315 |
| Tau1474 | LC | PRT | US20190112362; SEQ ID NO: 589 | 13316 |
| Tau1475 | HC | PRT | US20190112362; SEQ ID NO: 590 | 13317 |
| Tau1476 | LC | PRT | US20190112362; SEQ ID NO: 598 | 13318 |
| Tau1477 | LC | PRT | US20190112362; SEQ ID NO: 599 | 13319 |
| Tau1478 | LC | PRT | US20190112362; SEQ ID NO: 600 | 13320 |
| Tau1479 | LC | PRT | US20190112362; SEQ ID NO: 601 | 13321 |
| Tau1480 | HC | PRT | US20190112362; SEQ ID NO: 602 | 13322 |
| Tau1481 | VH | PRT | US20190112364; SEQ ID NO: 192 | 13323 |
| Tau1482 | VH | PRT | US20190112364; SEQ ID NO: 193 | 13324 |
| Tau1483 | VH | PRT | US20190112364; SEQ ID NO: 196 | 13325 |
| Tau1484 | HC | PRT | US20190112364; SEQ ID NO: 201 | 13326 |
| Tau1485 | HC | PRT | US20190112364; SEQ ID NO: 202 | 13327 |
| Tau1486 | VH | PRT | US20190112364; SEQ ID NO: 205 | 13328 |
| Tau1487 | HC | PRT | US20190112364; SEQ ID NO: 210 | 13329 |
| Tau1488 | HC | PRT | US20190112364; SEQ ID NO: 211 | 13330 |
| Tau1489 | VH | PRT | US20190112364; SEQ ID NO: 214 | 13331 |
| Tau1490 | HC | PRT | US20190112364; SEQ ID NO: 219 | 13332 |
| Tau1491 | HC | PRT | US20190112364; SEQ ID NO: 220 | 13333 |
| Tau1492 | VH | PRT | US20190112364; SEQ ID NO: 223 | 13334 |
| Tau1493 | HC | PRT | US20190112364; SEQ ID NO: 228 | 13335 |
| Tau1494 | HC | PRT | US20190112364; SEQ ID NO: 229 | 13336 |
| Tau1495 | VH | PRT | US20190112364; SEQ ID NO: 232 | 13337 |
| Tau1496 | HC | PRT | US20190112364; SEQ ID NO: 237 | 13338 |
| Tau1497 | HC | PRT | US20190112364; SEQ ID NO: 238 | 13339 |
| Tau1498 | VH | PRT | US20190112364; SEQ ID NO: 241 | 13340 |
| Tau1499 | HC | PRT | US20190112364; SEQ ID NO: 246 | 13341 |
| Tau1500 | HC | PRT | US20190112364; SEQ ID NO: 247 | 13342 |
| Tau1501 | VH | PRT | US20190112364; SEQ ID NO: 250 | 13343 |
| Tau1502 | HC | PRT | US20190112364; SEQ ID NO: 255 | 13344 |
| Tau1503 | HC | PRT | US20190112364; SEQ ID NO: 256 | 13345 |
| Tau1504 | VH | PRT | US20190112364; SEQ ID NO: 259 | 13346 |
| Tau1505 | HC | PRT | US20190112364; SEQ ID NO: 264 | 13347 |
| Tau1506 | HC | PRT | US20190112364; SEQ ID NO: 265 | 13348 |
| Tau1507 | VH | PRT | US20190112364; SEQ ID NO: 268 | 13349 |
| Tau1508 | HC | PRT | US20190112364; SEQ ID NO: 273 | 13350 |
| Tau1509 | HC | PRT | US20190112364; SEQ ID NO: 274 | 13351 |
| Tau1510 | VH | PRT | US20190112364; SEQ ID NO: 277 | 13352 |
| Tau1511 | HC | PRT | US20190112364; SEQ ID NO: 282 | 13353 |
| Tau1512 | HC | PRT | US20190112364; SEQ ID NO: 283 | 13354 |
| Tau1513 | VH | PRT | US20190112364; SEQ ID NO: 286 | 13355 |
| Tau1514 | HC | PRT | US20190112364; SEQ ID NO: 291 | 13356 |
| Tau1515 | HC | PRT | US20190112364; SEQ ID NO: 292 | 13357 |
| Tau1516 | VH | PRT | US20190112364; SEQ ID NO: 295 | 13358 |
| Tau1517 | HC | PRT | US20190112364; SEQ ID NO: 300 | 13359 |
| Tau1518 | HC | PRT | US20190112364; SEQ ID NO: 301 | 13360 |
| Tau1519 | VH | PRT | US20190112364; SEQ ID NO: 304 | 13361 |
| Tau1520 | HC | PRT | US20190112364; SEQ ID NO: 309 | 13362 |
| Tau1521 | HC | PRT | US20190112364; SEQ ID NO: 310 | 13363 |
| Tau1522 | VH | PRT | US20190112364; SEQ ID NO: 313 | 13364 |
| Tau1523 | HC | PRT | US20190112364; SEQ ID NO: 317 | 13365 |
| Tau1524 | HC | PRT | US20190112364; SEQ ID NO: 318 | 13366 |
| Tau1525 | VH | PRT | US20190112364; SEQ ID NO: 326 | 13367 |
| Tau1526 | HC | PRT | US20190112364; SEQ ID NO: 327 | 13368 |
| Tau1527 | HC | PRT | US20190112364; SEQ ID NO: 330 | 13369 |
| Tau1528 | VH | PRT | US20190112364; SEQ ID NO: 335 | 13370 |
| Tau1529 | HC | PRT | US20190112364; SEQ ID NO: 336 | 13371 |
| Tau1530 | HC | PRT | US20190112364; SEQ ID NO: 339 | 13372 |
| Tau1531 | HC | PRT | US20190112364; SEQ ID NO: 344 | 13373 |
| Tau1532 | HC | PRT | US20190112364; SEQ ID NO: 345 | 13374 |
| Tau1533 | VH | PRT | US20190112364; SEQ ID NO: 348 | 13375 |
| Tau1534 | HC | PRT | US20190112364; SEQ ID NO: 353 | 13376 |
| Tau1535 | HC | PRT | US20190112364; SEQ ID NO: 354 | 13377 |
| Tau1536 | VH | PRT | US20190112364; SEQ ID NO: 357 | 13378 |
| Tau1537 | HC | PRT | US20190112364; SEQ ID NO: 362 | 13379 |
| Tau1538 | HC | PRT | US20190112364; SEQ ID NO: 363 | 13380 |
| Tau1539 | VH | PRT | US20190112364; SEQ ID NO: 366 | 13381 |
| Tau1540 | HC | PRT | US20190112364; SEQ ID NO: 371 | 13382 |
| Tau1541 | HC | PRT | US20190112364; SEQ ID NO: 372 | 13383 |
| Tau1542 | VH | PRT | US20190112364; SEQ ID NO: 375 | 13384 |
| Tau1543 | HC | PRT | US20190112364; SEQ ID NO: 380 | 13385 |
| Tau1544 | HC | PRT | US20190112364; SEQ ID NO: 381 | 13386 |
| Tau1545 | VH | PRT | US20190112364; SEQ ID NO: 384 | 13387 |
| Tau1546 | HC | PRT | US20190112364; SEQ ID NO: 389 | 13388 |
| Tau1547 | HC | PRT | US20190112364; SEQ ID NO: 390 | 13389 |
| Tau1548 | VH | PRT | US20190112364; SEQ ID NO: 393 | 13390 |
| Tau1549 | HC | PRT | US20190112364; SEQ ID NO: 398 | 13391 |
| Tau1550 | HC | PRT | US20190112364; SEQ ID NO: 399 | 13392 |
| Tau1551 | VH | PRT | US20190112364; SEQ ID NO: 402 | 13393 |
| Tau1552 | LC | PRT | US20190112364; SEQ ID NO: 407 | 13394 |
| Tau1553 | LC | PRT | US20190112364; SEQ ID NO: 408 | 13395 |
| Tau1554 | VL | PRT | US20190112364; SEQ ID NO: 411 | 13396 |
| Tau1555 | LC | PRT | US20190112364; SEQ ID NO: 416 | 13397 |
| Tau1556 | LC | PRT | US20190112364; SEQ ID NO: 417 | 13398 |
| Tau1557 | VL | PRT | US20190112364; SEQ ID NO: 420 | 13399 |
| Tau1558 | LC | PRT | US20190112364; SEQ ID NO: 425 | 13400 |
| Tau1559 | LC | PRT | US20190112364; SEQ ID NO: 426 | 13401 |
| Tau1560 | VL | PRT | US20190112364; SEQ ID NO: 429 | 13402 |
| Tau1561 | LC | PRT | US20190112364; SEQ ID NO: 434 | 13403 |
| Tau1562 | LC | PRT | US20190112364; SEQ ID NO: 435 | 13404 |
| Tau1563 | VL | PRT | US20190112364; SEQ ID NO: 438 | 13405 |
| Tau1564 | LC | PRT | US20190112364; SEQ ID NO: 443 | 13406 |
| Tau1565 | LC | PRT | US20190112364; SEQ ID NO: 444 | 13407 |
| Tau1566 | VL | PRT | US20190112364; SEQ ID NO: 447 | 13408 |
| Tau1567 | LC | PRT | US20190112364; SEQ ID NO: 452 | 13409 |
| Tau1568 | LC | PRT | US20190112364; SEQ ID NO: 453 | 13410 |
| Tau1569 | VL | PRT | US20190112364; SEQ ID NO: 456 | 13411 |
| Tau1570 | LC | PRT | US20190112364; SEQ ID NO: 461 | 13412 |
| Tau1571 | LC | PRT | US20190112364; SEQ ID NO: 462 | 13413 |
| Tau1572 | VL | PRT | US20190112364; SEQ ID NO: 465 | 13414 |
| Tau1573 | LC | PRT | US20190112364; SEQ ID NO: 470 | 13415 |
| Tau1574 | LC | PRT | US20190112364; SEQ ID NO: 471 | 13416 |
| Tau1575 | VL | PRT | US20190112364; SEQ ID NO: 474 | 13417 |
| Tau1576 | LC | PRT | US20190112364; SEQ ID NO: 479 | 13418 |
| Tau1577 | LC | PRT | US20190112364; SEQ ID NO: 480 | 13419 |
| Tau1578 | VL | PRT | US20190112364; SEQ ID NO: 483 | 13420 |
| Tau1579 | LC | PRT | US20190112364; SEQ ID NO: 487 | 13421 |
| Tau1580 | LC | PRT | US20190112364; SEQ ID NO: 488 | 13422 |
| Tau1581 | VL | PRT | US20190112364; SEQ ID NO: 491 | 13423 |
| Tau1582 | LC | PRT | US20190112364; SEQ ID NO: 496 | 13424 |
| Tau1583 | LC | PRT | US20190112364; SEQ ID NO: 497 | 13425 |
| Tau1584 | VL | PRT | US20190112364; SEQ ID NO: 500 | 13426 |
| Tau1585 | LC | PRT | US20190112364; SEQ ID NO: 505 | 13427 |
| Tau1586 | LC | PRT | US20190112364; SEQ ID NO: 506 | 13428 |
| Tau1587 | VL | PRT | US20190112364; SEQ ID NO: 509 | 13429 |
| Tau1588 | LC | PRT | US20190112364; SEQ ID NO: 514 | 13430 |
| Tau1589 | LC | PRT | US20190112364; SEQ ID NO: 515 | 13431 |
| Tau1590 | VL | PRT | US20190112364; SEQ ID NO: 518 | 13432 |
| Tau1591 | LC | PRT | US20190112364; SEQ ID NO: 523 | 13433 |
| Tau1592 | LC | PRT | US20190112364; SEQ ID NO: 524 | 13434 |
| Tau1593 | VL | PRT | US20190112364; SEQ ID NO: 527 | 13435 |
| Tau1594 | LC | PRT | US20190112364; SEQ ID NO: 532 | 13436 |
| Tau1595 | LC | PRT | US20190112364; SEQ ID NO: 533 | 13437 |
| Tau1596 | VL | PRT | US20190112364; SEQ ID NO: 536 | 13438 |
| Tau1597 | LC | PRT | US20190112364; SEQ ID NO: 541 | 13439 |
| Tau1598 | LC | PRT | US20190112364; SEQ ID NO: 542 | 13440 |
| Tau1599 | VL | PRT | US20190112364; SEQ ID NO: 545 | 13441 |
| Tau1600 | LC | PRT | US20190112364; SEQ ID NO: 550 | 13442 |
| Tau1601 | LC | PRT | US20190112364; SEQ ID NO: 551 | 13443 |
| Tau1602 | VL | PRT | US20190112364; SEQ ID NO: 554 | 13444 |
| Tau1603 | LC | PRT | US20190112364; SEQ ID NO: 559 | 13445 |
| Tau1604 | LC | PRT | US20190112364; SEQ ID NO: 560 | 13446 |
| Tau1605 | VL | PRT | US20190112364; SEQ ID NO: 563 | 13447 |
| Tau1606 | LC | PRT | US20190112364; SEQ ID NO: 568 | 13448 |
| Tau1607 | LC | PRT | US20190112364; SEQ ID NO: 569 | 13449 |
| Tau1608 | VL | PRT | US20190112364; SEQ ID NO: 572 | 13450 |
| Tau1609 | LC | PRT | US20190112364; SEQ ID NO: 577 | 13451 |
| Tau1610 | LC | PRT | US20190112364; SEQ ID NO: 578 | 13452 |
| Tau1611 | VL | PRT | US20190112364; SEQ ID NO: 581 | 13453 |
| Tau1612 | LC | PRT | US20190112364; SEQ ID NO: 586 | 13454 |
| Tau1613 | LC | PRT | US20190112364; SEQ ID NO: 587 | 13455 |
| Tau1614 | VL | PRT | US20190112364; SEQ ID NO: 590 | 13456 |
| Tau1615 | VH | PRT | WO2018154390; SEQ ID NO: 18 | 13457 |
| Tau1616 | VH | PRT | WO2018154390; SEQ ID NO: 20 | 13458 |
| Tau1617 | VH | PRT | WO2018154390; SEQ ID NO: 22 | 13459 |
| Tau1618 | VH | PRT | WO2018154390; SEQ ID NO: 24 | 13460 |
| Tau1619 | VH | PRT | WO2018154390; SEQ ID NO: 26 | 13461 |
| Tau1620 | VH | PRT | WO2018154390; SEQ ID NO: 28 | 13462 |
| Tau1621 | VH | PRT | WO2018154390; SEQ ID NO: 30 | 13463 |
| Tau1622 | VL | PRT | WO2018154390; SEQ ID NO: 32 | 13464 |
| Tau163 | VL | PRT | WO2018154390; SEQ ID NO: 34 | 13465 |
| Tau1624 | VL | PRT | WO2018154390; SEQ ID NO: 36 | 13466 |
| Tau1625 | VL | PRT | WO2018154390; SEQ ID NO: 38 | 13467 |
| Tau1626 | VL | PRT | WO2018154390; SEQ ID NO: 40 | 13468 |
| Tau1627 | VL | PRT | WO2018154390; SEQ ID NO: 42 | 13469 |
| Tau1628 | LC | PRT | WO2018154390; SEQ ID NO: 44 | 13470 |
| Tau1629 | HC | PRT | WO2018154390; SEQ ID NO: 46 | 13471 |
| Tau1630 | LC | PRT | WO2018154390; SEQ ID NO: 48 | 13472 |
| Tau1631 | HC | PRT | WO2018154390; SEQ ID NO: 50 | 13473 |
| Tau1632 | VL | PRT | WO2018154390; SEQ ID NO: 84 | 13474 |
| Tau1633 | LC | PRT | WO2018154390; SEQ ID NO: 85 | 13475 |
| Tau1634 | VH | PRT | WO2018154390; SEQ ID NO: 89 | 13476 |
| Tau1635 | VL | PRT | WO2018154390; SEQ ID NO: 90 | 13477 |
| Tau1636 | VL | PRT | WO2018154390; SEQ ID NO: 105 | 13478 |
| Tau1637 | VL | PRT | WO2018154390; SEQ ID NO: 106 | 13479 |
| Tau1638 | VL | PRT | WO2018154390; SEQ ID NO: 107 | 13480 |
| Tau1639 | VL | PRT | WO2018154390; SEQ ID NO: 108 | 13481 |
| Tau1640 | VL | PRT | WO2018154390; SEQ ID NO: 109 | 13482 |
| Tau1641 | VL | PRT | WO2018154390; SEQ ID NO: 110 | 13483 |
| Tau1642 | VL | PRT | WO2018154390; SEQ ID NO: 111 | 13484 |
| Tau1643 | VL | PRT | WO2018154390; SEQ ID NO: 112 | 13485 |
| Tau1644 | VL | PRT | WO2018154390; SEQ ID NO: 113 | 13486 |
| Tau1645 | VL | PRT | WO2018154390; SEQ ID NO: 114 | 13487 |
| Tau1646 | VH | PRT | WO2018154390; SEQ ID NO: 127 | 13488 |
| Tau1647 | VH | PRT | WO2018154390; SEQ ID NO: 128 | 13489 |
| Tau1648 | VH | PRT | WO2018154390; SEQ ID NO: 129 | 13490 |
| Tau1649 | LC | PRT | WO2018154390; SEQ ID NO: 130 | 13491 |
| Tau1650 | LC | PRT | WO2018154390; SEQ ID NO: 131 | 13492 |
| Tau1651 | LC | PRT | WO2018154390; SEQ ID NO: 132 | 13493 |
| Tau1652 | HC | PRT | WO2018154390; SEQ ID NO: 133 | 13494 |
| Tau1653 | HC | PRT | WO2018154390; SEQ ID NO: 134 | 13495 |
| Tau1654 | VL | PRT | WO2018154390; SEQ ID NO: 162 | 13496 |
| Tau1655 | VL | PRT | WO2018154390; SEQ ID NO: 163 | 13497 |
| Tau1656 | VL | PRT | WO2018154390; SEQ ID NO: 164 | 13498 |
| Tau1657 | VL | PRT | WO2018154390; SEQ ID NO: 165 | 13499 |
| Tau1658 | VL | PRT | WO2018154390; SEQ ID NO: 166 | 13500 |
| Tau1659 | VL | PRT | WO2018154390; SEQ ID NO: 167 | 13501 |
| Tau1660 | VL | PRT | WO2018154390; SEQ ID NO: 168 | 13502 |
| Tau1661 | VL | PRT | WO2018154390; SEQ ID NO: 169 | 13503 |
| Tau1662 | VL | PRT | WO2018154390; SEQ ID NO: 170 | 13504 |
| Tau1663 | VL | PRT | WO2018154390; SEQ ID NO: 171 | 13505 |
| Tau1664 | VL | PRT | WO2018154390; SEQ ID NO: 172 | 13506 |
| Tau1665 | VL | PRT | WO2018154390; SEQ ID NO: 173 | 13507 |
| Tau1666 | VL | PRT | WO2018154390; SEQ ID NO: 174 | 13508 |
| Tau1667 | VL | PRT | WO2018154390; SEQ ID NO: 175 | 13509 |
| Tau1668 | VL | PRT | WO2018154390; SEQ ID NO: 176 | 13510 |
| Tau1669 | VL | PRT | WO2018154390; SEQ ID NO: 177 | 13511 |
| Tau1670 | VL | PRT | WO2018154390; SEQ ID NO: 178 | 13512 |
| Tau1671 | VL | PRT | WO2018154390; SEQ ID NO: 179 | 13513 |
| Tau1672 | VL | PRT | WO2018154390; SEQ ID NO: 180 | 13514 |
| Tau1673 | VL | PRT | WO2018154390; SEQ ID NO: 181 | 13515 |
| Tau1674 | VL | PRT | WO2018154390; SEQ ID NO: 182 | 13516 |
| Tau1675 | VL | PRT | WO2018154390; SEQ ID NO: 183 | 13517 |
| Tau1676 | VL | PRT | WO2018154390; SEQ ID NO: 185 | 13518 |

In one embodiment, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 11. As another non-limiting example, the antibody may be one or more of the heavy chain sequences listed in Table 11. As a non-limiting example, the antibody may be one or more of the light chain sequences listed in Table 11.

In one embodiment, the payload region of the AAV particle comprises a nucleic acid sequence encoding a polypeptide comprising a heavy chain and a light chain sequence listed in Table 11. The payload region may also comprise a linker between the heavy and light chain sequences. The linker may be a sequence known in the art or described in Table 2.

In one embodiment, the payload region of the AAV particle comprises a nucleic acid sequence encoding a polypeptide comprising a heavy chain and alight chain sequence listed in Table 11, where the heavy chain sequence is from a different antibody than the light chain sequence. The payload region may also comprise a linker between the heavy and light chain sequences. The linker may be a sequence known in the art or described in Table 2.

In one embodiment, the payload region comprises, in the 5 to 3 direction, an antibody light chain sequence, a linker and a heavy chain sequence.

In one embodiment, the payload region comprises a nucleic acid sequence encoding, in the 5 to 3′ direction, an antibody light chain sequence from Table 11, one or more linkers from Table 2 and a heavy chain sequence from Table 11. Non-limiting examples are included in Table 4.

In one embodiment, the payload region comprises, in the 5 to 3 direction, an antibody heavy chain sequence, a linker region (may comprise one or more linkers) and alight chain sequence.

In one embodiment, the payload region comprises a nucleic acid sequence encoding, in the 5 to 3′ direction, an antibody heavy chain sequence from Table 11, one or more linkers from Table 2, and a light chain sequence from Table 11. Non-limiting examples are included in Table 11.

In one embodiment, the payload region comprises a nucleic acid sequence encoding a single heavy chain. As a non-limiting example, the heavy chain is an amino acid sequence or fragment thereof described in Table 11.

Shown in Table 11 are a listing of antibodies and their polynucleotides and/or polypeptides sequences. These sequences may be encoded by or included in the AAV particles of the present invention. Variants or fragments of the antibody sequences described in Table 11 may be utilized in the AAV particles of the present invention.

In some embodiments, the AAV particles may comprise codon-optimized versions of the nucleic acids encoding the polypeptides listed in Table 11. In some cases, the payload region of the AAV particles of the invention may encode one or more isoforms or variants of these heavy and light chain antibody domains. Such variants may be humanized or optimized antibody domains comprising one or more complementarity determining regions (CDRs) from the heavy and light chains listed in Table 11. CDRs of the antibodies encoded by the viral genomes of the present invention may be 50%,60%,70%,80%, 90%, 95% identical to CDRs listed in or incorporated in the sequences of Table 11. Methods of determining CDRs are well known in the art and are described herein. Payload regions may encode antibody variants with one or more heavy chain variable domain (V_{H}) or light chain variable domain (V_{L}) derived from the antibody sequences in Table 11. In some cases, such variants may include bispecific antibodies. Bispecific antibodies encoded by payload regions of the invention may comprise variable domain pairs from two different antibodies.

In one embodiment, the AAV particles may comprise a heavy and alight chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in its entirety. As another non-limiting example, the AAV particles may be a dual-promoter V for antibody expression as described by Lewis et al. (J. of. Virology, September 2002, Vol. 76(17), p 8769-8775; the contents of which are herein incorporated by reference in its entirety).

Payload regions of the viral genomes of the invention may encode any anti-tau antibodies, or tau-associated antibodies, not limited to those described in Table 11, including antibodies that are known in the art and/or antibodies that are commercially available. This may include fragments of such antibodies or antibodies that have been developed to comprise one or more of such fragments [e.g., variable domains or complementarity determining regions (CDRs)]. Anti-tau antibodies that may be encoded by payloads of the invention include, but are not limited to, AT8 (pSer²⁰²/pThr²⁰²; ThermoFisher, Waltham, Mass.; described in International Publication No. WO1995017429, the contents of which are herein incorporated in their entirety), AT100 (pSer²¹²/pSer²¹⁴; ThermoFisher, Waltham, Mass.; described in U.S. Pat. No. 6,121,003, the contents of which are herein incorporated in their entirety), AT180 (pThr²³¹; ThermoFisher, Waltham, Mass.; described in International Publication No. WO1995017429, the contents of which are herein incorporated by reference in their entirety), MC-1 (Tau^{2-18/312-342} conformational antibody; as described in International Publication WO199620218, the contents of which are herein incorporated by reference in their entirety), MC-6 (pSer²³⁵; described in U.S. Pat. No. 5,811,310, the contents of which are herein incorporated in their entirety), TG-3 (pThr²³¹; described in Jicha, G A et al., 1997 J Neurochem 69(5):2087-95, the contents of which are herein incorporated by reference in their entirety), CP13 (pSer²⁰²), CP27 (human Tau¹³⁰⁻¹⁵⁰), Tau12 (human Tau⁹⁻¹⁸; Abcam, Cambridge, Mass.), TG5 (Tau²²⁰⁻²⁴²; described in U.S. Pat. No. 5,811,310), DA9 (Tau¹⁰²⁻¹⁴⁰; described in U.S. Pat. No. 5,811,310), PHF-1 (pSer³⁹⁵/pSer⁴⁰⁴; described in International Publication WO199620218), Alz50 (Tau⁷⁻⁹ and Tau³¹²⁻³⁴² conformational epitope; described in U.S. Pat. No. 5,811,310 and Carmel, G et al 1996 J Biol Chem 271(51):32780-32795 and Jicha, G A et al, 1997 J Neurosci Res 48(2):128-132, the contents of each of which are herein incorporated by reference in their entirety), Tau-1 (de-phosphorylated Ser¹⁹⁵/Ser¹⁹⁸/Ser¹⁹⁹/Ser²⁰²; ThermoFisher, Waltham, Mass.), Tau46 (Tau⁴⁰⁴⁻⁴⁴¹; Abcam, Cambridge, Mass.), pS199 (ThermoFisher, Waltham, Mass.), pT205, pS396 (ThermoFisher, Waltham, Mass.; described in U.S. Pat. No. 8,647,631, the contents of which are herein incorporated by reference in their entirety), pS404 (ThermoFisher, Waltham, Mass.; described in U.S. Pat. No. 8,647,631, the contents of which are herein incorporated by reference in their entirety), pS422 (ThermoFisher, Waltham, Mass.), A0024 (hTau²⁴³⁻⁴⁴¹; Dako, Glostrup, Denmark), HT7 (hTau¹⁵⁹⁻¹⁶³; ThermoFisher, Waltham, Mass.), Tau2 (hTau⁵²⁻⁶⁸; Abcam, Cambridge, Mass.), AD2 (pSer³⁹⁶/pSer⁴⁰⁴; Bio-Rad Laboratories, Hercules, Calif.), AT120 (hTau²¹⁶⁻²²⁴; described in U.S. Pat. No. 5,843,779, the contents of which are herein incorporated by reference in their entirety), AT270 (pThr¹⁸¹; ThermoFisher, Waltham, Mass.), 12E8 (pSer²⁶² and/or Ser³⁵⁶), K9JA (hTau²⁴³⁻⁴⁴¹; Dako, Caprinteria, Calif.), TauC3 (hTau Asp441; Santa Cruz Biotechnology, Dallas, Tex.; described in United States Patent Publication US20120244174 and Gamblin, T C et al 2003 PNAS 100(17):10032-7, the contents of each of which are herein incorporated by reference in their entirety), 4E6G7 (pSer³⁹⁶/pSer⁴⁰⁴; described in United States Patent Publication No. US2010316564 and Congdon, E. E. et al., 2016. Molecular Neurodegeneration August 30; 11(1):62, the contents of which are herein incorporated by reference in their entirety), 682 and variants thereof, described in International Patent Publication WO2016007414, the contents of which are herein incorporated by reference in their entirety, RZ3 (pThr²³¹), PG5 (pSer⁴⁰⁹), BT2 (pS^{199/202}), DA31 (Tau¹⁵⁰⁻¹⁹⁰), CP9 (pThr²³¹) Ta1505 (phospho site between Tau⁴¹⁰⁻⁴²¹, particularly pSer⁴¹³ as described in United States Patent Publication US20150183854 and Umeda, T. et al., 2015. Ann Clin Trans Neurol 2(3): 241-255, the contents of each of which are herein incorporated by reference in their entirety), PHF-6 (pThr²³¹, as described in Hoffman R et al., 1997. Biochemistry 36; 8114-8124, the contents of which are herein incorporated by reference in their entirety), PHF-13 (pSer³⁹⁶, as described in Hoffman R et al., 1997. Biochemistry 36; 8114-8124), 1685 (Tau²⁵⁻⁴⁶, as described in United States Publication US20160031976, the contents of which are herein incorporated by reference in their entirety), DC8E8 (as described in United States Patent Publication US20150050215, the contents of which are herein incorporated by reference in their entirety), PT1 or PT3 (as described in U.S. Pat. No. 9,371,376, the contents of which are herein incorporated by reference in their entirety), 4G11 (Tau⁵⁷⁻⁶⁴ as described in International Publication WO2016137950, the contents of which are herein incorporated by reference in their entirety), 1A6 (Tau⁷⁻¹⁷ and Tau²¹⁵⁻²²⁰, as described in International Publication WO2016137950), Tau15 or Tau81 (as described in International Publication WO2016055941, the contents of which are herein incorporated by reference in their entirety), TOC-1 (dimerized or aggregated tau, as described in International Publication WO2012149365, the contents of which are herein incorporated by reference in their entirety), pS404lgG2a/k (Neotope Biosciences, South San Francisco, Calif.; as described in Ittner et al., 2015. Neurochemistry 132:135-145, the contents of which are herein incorporated by reference in their entirety), TOMA (tau oligomer monoclonal antibody; as described in Nos. U.S. Pat. Nos. 8,778,343 and 9,125,846, International Publications WO2012051498 and WO2011026031, or United States Publication Nos. US20150004169 and US20150322143, and Castillo-Carranza, D L et al., 2014 J Neurosci 34(12)4260-72, the contents of each of which are herein incorporated by reference in their entirety), TTC-99 (oligomeric tau), BMS-986168 (as described in United States Patent Publication US2014294831, International Publication WO2015081085 and U.S. Pat. No. 8,980,271, the contents of which are herein incorporated by reference in their entirety), 3H3 (pan-amyloid epitope; described in Levites, Y et al 2015 J Neurosci 35(16)6265-76, the contents of which are herein incorporated by reference in their entirety), cis-pT231 (described in International Publications WO2012149334 and WO2011056561, the contents of which are herein incorporated by reference in their entirety), CP-3 (pSer²¹⁴; described in Jicha et al 1999 J Neurosci 19(17):7486-94, the contents of which are herein incorporated by reference in their entirety), TNT1 (Tau²⁻¹⁸; as described in United States Patent Publication 20160031978, the contents of which are herein incorporated by reference in their entirety), Tau-nY29 (nTyr²⁹; described in Reynolds M R, et al., 2006 J Neurosci 26(42):10636-45, the contents of which are herein incorporated by reference in their entirety), Tau-nY197 (nTyr¹⁹⁷; described in Reyes, J F et al., 2012 Acta Neuropathol 123(1):119-32, the contents of which are herein incorporated by reference in their entirety), Tau-nY394 (nTyr³⁹⁴; described in Reyes, J F et al 2012), 4E4 (Tau³³⁷⁻³⁴³ Tau³³⁷⁻³⁹⁷; described in International Publication WO2012049570 and United States Patent Publication US20150252102, the contents of each of which are herein incorporated by reference in their entirety), ADx210 (described in United States Patent Publication US20140161875, the contents of which are herein incorporated by reference in their entirety), ADx215 (described in United States Patent Publication US20140161875), ADx202 (as described in International Publication WO2015004163, the contents of which are herein incorporated by reference in their entirety), AP422 (pSer⁴²²; described in Hasegawa, M et al 1996 FEBS Lett 384:25-30, the contents of which are herein incorporated by reference in their entirety), Tau5 (Tau²¹⁰⁻²⁴¹), RTA2 (Tau²⁷³⁻²⁸³), RTAC (Tau⁴²⁶⁻⁴⁴¹), RTA1 (Tau²⁵⁷⁻²⁷⁴), T46 (Tau³⁹⁵⁻⁴³²), T49, MIGT4, O.BG.15, 525, 3-39, 4F1, MapTau (Tau⁹⁵⁻¹⁰⁸; SMI Covance), T1, HYB33801 (Tau⁵⁻¹²), Tau13 (Tau²⁻¹⁸), B11E8, 5J20 (14-3-3 tau), DC25 (Tau³⁴⁷⁻³⁵³), DC39N1 (Tau⁴⁵⁻⁷³), DC-11 (Tau³²¹⁻³⁹¹; described in U.S. Pat. No. 7,746,180, the contents of which are herein incorporated by reference in their entirety), DC39 (Tau⁴⁰¹⁻⁴¹¹), DC4R, n847 (nitrated tau), SPM452, TI4, 1E1/A6 (Tau²⁷⁵⁻²⁹¹), 5E2, 8E6/C11 (Tau²⁰⁹⁻²²⁴), 2E12 (pT231), NFT200, 248E5 (Tau³⁻²¹⁴), IG2 (Thr¹⁷⁵, Thr¹⁸¹, Thr²³¹; as described in International Publication WO2016041553, the contents of which are herein incorporated by reference in their entirety), YP3 (as described in WO2007019273, the contents of which are herein incorporated by reference in their entirety), YP4 (as described in WO2007019273) and 14-3-3 Tau (pSer 14-3-3 binding motif; Abcam, Cambridge, Mass.). Further, anti-tau antibodies may be any of those listed in the antibody section of Alzforum.org or at the Antibody Resource Page.com, the contents of each of which are herein incorporated by reference in their entirety. Further, anti-tau antibodies may be any commercially available anti-tau antibody. Additional antibodies may include any of those taught in Petry, F. R. et al., 2014. PLoS One 9(5): e94251, the contents of which are herein incorporated by reference in their entirety. In one example, such antibodies may include any of those described in Jicha, G. A. et al., 1997. Journal of Neuroscience Research 48:128-132, the contents of which are herein incorporated by reference in their entirety. One such antibody, MC-1, recognizes distinct conformations of tau that are associated with neurological disease.

In some embodiments, payloads may encode anti-tau antibodies (or fragments thereof) taught in United States Publication No. US2014294831, the contents of which are herein incorporated by reference in their entirety. Such antibodies may include IPN001 and/or IPN002 antibodies or fragments of such antibodies. In some cases, variable domains of IPN002 as presented in FIGS. 2A and 2B of US2014294831 may be used (e.g., incorporated into another antibody). In some cases, CDR regions of IPN002 as underlined in FIGS. 2A and 28 may be used (e.g., incorporated into another antibody or used to prepare humanized versions of IPN002). In some cases, anti-tau antibodies may include any of the IPN001 or IPN002 antibody variants taught in US2014294831 (e.g., in FIGS. 9-16 of that publication). In one embodiment, this antibody is also referred to as BMS. 986168.

In some cases, payloads may encode anti-tau antibodies (or fragments thereof) taught in Otvos, L et al., 1994. J Neurosci. Res 39(6):669-73, the contents of which are herein incorporated by reference in their entirety. Such antibodies may include monoclonal antibody PHF-1 or fragments thereof. The PHF-1 antibody binds to tau paired helical filaments, a pathological conformation of tau, found in certain neurological disorders, including Alzheimer's disease. Further, antibody affinity is increased when either serine 396 or serine 404 of tau is phosphorylated and even further increased when both are phosphorylated.

In some embodiments, payloads may encode anti-tau antibodies (or fragments thereof) taught in U.S. Pat. No. 5,811,310, the contents of which are herein incorporated by reference in their entirety. Such embodiments may include monoclonal antibodies PHF-1 or MC-1 or fragments thereof. MC-1 is a conformational antibody binding to the epitopes presented in Jicha, G A., et al., 1997 J Neurosci Res 48(128.132).

In some embodiments, payloads may encode anti-tau antibodies (or fragments thereof) taught in International Publication Number WO2015035190, the contents of which are herein incorporated by reference in their entirety. Such embodiments may include, but are not limited to, antibodies PHF-1 or MC-1 or fragments thereof. Viral genomes of the V particles of the present invention may comprise or encode any of SEQ ID NO: 1-6 of WO2015035190.

Anti-tau antibodies (or fragments thereof) encoded by viral genomes of the invention may include antibodies that bind to one or more of the epitopes presented in Otvos, L. et al., 1994. J Neurosci. Res 39(6):669-73 (e.g., any of those presented in Table 1 of that publication).

In some embodiments, payloads may encode anti-tau antibodies (or fragments thereof) taught in U.S. Pat. No. 7,746,180, the contents of which are herein incorporated by reference in their entirety. Such embodiments may include antibody DC-11 or fragments thereof.

In some embodiments, the antibodies encoded by the viral genomes of the present invention may target any of the antigenic regions or epitopes described in United States Patent Publication No US2008050383 or US20100316564, the contents of which are herein incorporated by reference in their entirety. In one embodiment, the antibody targets pS396/pS404. Such embodiments may include antibody 4E6 and/or variants or fragments thereof. The affinity of antibody 4E6 for soluble PHF and its ability to reduce soluble phospho tau has been described in Congdon, E E. et al., 2016. Molecular Neurodegeneration August 30; 11(1):62, the contents of which are herein incorporated by reference in their entirety.

In some embodiments, the antibodies encoded by the viral genomes of the present invention may target any of the antigenic regions or epitopes described in International Patent Publication WO1998022120, the contents of which are herein incorporated by reference in their entirety. In one embodiment, the antibody may be PHF-6 (pT231), or fragments or variants thereof. In another embodiment, the antibody may be PHF-13 (pS396), or a fragment of variant thereof. These antibodies are further described in Hoffman et al., 1997. Biochemistry 36: 8114.8124, the contents of which are herein incorporated by reference in their entirety.

In some embodiments, the antibodies encoded by the viral genomes of the present invention may target any of the antigenic regions or epitopes described in International Publication WO2016126993, the contents of which are herein incorporated by reference in their entirety. The antibodies may be derived from any of the tau epitopes described in Table A of WO2016126993. In one embodiment, the antibody of the present invention may comprise any of the sequences listed in Table B or Table 1 of WO2016126993.

In some embodiments, the antibodies encoded by the viral genomes of the present invention may target any of the antigenic regions or epitopes described in United States Patent Publication US20120244174, the contents of which are herein incorporated by reference in their entirety. In one embodiment, the antibody may bind to caspase-cleaved tau. In one embodiment, the epitope for antibodies targeting caspase cleaved tau is aspartic acid 421. In another embodiment, the epitope for antibodies targeting caspase cleaved tau may be the C-terminus after glutamic residue Glu391. In yet another embodiment, the epitope for antibodies targeting caspase cleaved tau may be at the N-terminus at aspartic acid residue 13. In another embodiment, the antibody may be TauC3.

In some embodiments, the antibodies encoded by the viral genomes of the present invention may target any of the antigenic regions or epitopes described in United States Patent Publication US20160031978, the contents of which are herein incorporated by reference in their entirety. In one embodiment, the antibody may bind to tau N-terminal residues associated with the PP1/GSK3 signaling cascade. In one embodiment, the antibody may be TNT1.

In some embodiments, the antibodies encoded by the viral genomes of the present invention may be any of those described in d'Abramo, C et al., 2015. PLOS One 10(8):e0135774, the contents of which are herein incorporated by reference in their entirety. In one embodiment, the antibody may be CP13 (pS202), or a fragment or variant thereof. In another embodiment, the antibody may be RZ3 (pT231), or a fragment or variant thereof. In another embodiment, the antibody may be PG5 (pS409), or a fragment or variant thereof.

Anti-tau antibodies or fragments thereof encoded by the viral genomes of the present invention may target tau in any antigenic form. As non-limiting examples, antigenic tau may be an unphosphorylated or unmodified tau protein, a phosphorylated or otherwise post-translationally modified tau protein (O-GlnAcylated, or nitrosylated), an oligomeric species of tau protein, a soluble species of tau protein, an insoluble species of tau protein, a conformationally abnormal species of tau protein, a neuropathological form of tau protein and/or a neurofibrillary tangle or a precursor thereof.

Anti-tau antibodies or fragments thereof encoded by the viral genomes of the invention, may target any antigenic region or epitope along the full length of any of the six human tau protein isoforms. As non-limiting examples, the targeted antigenic peptides of the tau protein may be any of the following phosphorylated sites pT50, pS396, pS396-pS404, pS404, pS396-pS404-pS422, pS409, pS413, pS422, pS198, pS199, pS199-pS202, pS202, pT205, pT212, pS214, pT212-pS214, pT181, pT231, cis-pT231, pS235, pS238, pT245, pS262, pY310, pY394, pS324, pS356, pTau¹⁷⁷⁻¹⁸⁷, pY18, pS610, pS622, nitrosylated tau (nY18, nY29), methylated tau (di-meK281, dimeK311), O-GlnAcylated tau at S400, any of the following acetylated sites acK174, acK274, acK280, acK281 and/or any combination thereof. Acetylated tau proteins and associated antigenic peptides are described in Min et al, 2010, Neuron., 67, 953-966, Min et al., 2015, Nature Medicine., 10, 1154.1162, Cohen et al., 2011, Nature Communications., 2, 252, Gorsky et al., 2016, Scientific Report., 6, 22685, Tracy et al., 2016, Neuron., 90, 245-260, the contents of each of which are herein incorporated by reference in their entirety. Phosphorylated tau proteins and associated antigenic peptides are described in Asuni et al., 2007, J Neurosci., 27, 9115-9129, Boutajangout et al., 2010, J Neurosci., 30, 16559-16566, Boutajangout et al., 2011, J Neurochem., 118, 658-667, Chai et al., 2011, J Biol Chem., 286, 34457-34467, Gu et al., 2011, J Biol Chem., 288, 33081-33095, Sankaranarayanan et al., 2015, PloS One, 10, e0125614, Ittner et al., 2015, J Neurochem., 132, 135-145, D'Abramo et al., 2016, Neurobiol Aging, 37, 58-65, Collin et al., 2014, Brain., 137, 2834-2846, Kondo et al., 2015, Nature, 523, 431-436, the contents of each of which are herein incorporated by reference in their entirety.

In one embodiment, the antibody encoded by the viral genomes of the present invention may be a pS409 targeting antibody as described in Lee et al., 2016, Cell Reports, 16, 1690-1700, or International Patent Publication WO2013151762, the contents of each of which are herein incorporated by reference in their entirety. In some embodiments, this antibody may be RG6100 or R071057 or variants or fragments thereof.

In one embodiment, the antibody encoded by the viral genomes of the present invention may be a pS413 targeting antibody as described in Umeda et al., 2015, Ann Clin Trans Neurol., 2(3), 241.255 or International Patent Publication WO201310238, the contents of each of which are herein incorporated by reference in their entirety. In one embodiment, the antibody is Ta1505 or variants or fragments thereof.

In one embodiment, the antibody encoded by the viral genomes of the present invention may target a tau epitope with amino acid residues 210-275, more specifically pS238 and/or pT245, as described in International Publication WO2011053565, the contents of which are herein incorporated by reference in their entirety.

In one embodiment, the CDRs of an antibody encoded by the viral genomes of the present invention may be any of those listed in or incorporated in the antibody sequences of Table 11. In one embodiment, the CDRs may be any of those described in International Publication WO2015122922, the contents of which are herein incorporated by reference in their entirety.

In one embodiment, a CDR may be any of those chosen from the group of SEQ ID NO: 41, 49, or 57 of WO2015122922. Further a CDR of an antibody encoded by the viral genomes of the present invention may have 50%, 60%, 70%, 80%, 90%, or 95% identity to SEQ ID NO: 41, 49, or 57 of WO2015122922.

In one embodiment, the antibodies encoded by the viral genomes of the present invention may be any of those described in International Publication WO2016097315, the contents of which are herein incorporated by reference in their entirety. In one embodiment, the antibody may have an amino acid sequence as shown by SEQ ID NO: 2, 11, 20, 29, 38, 47, 56, 65, 74, 83, 92, 101, 110, 119, 128, 137, 146, 155, 164, 173, 182, 191, 209, 218, 226, or 227 of WO2016097315.

In one embodiment, the antibodies encoded by the viral genomes of the present invention may be a multispecific blood brain barrier receptor antibody that also targets tau, as described in International Publication WO2016094566, the contents of which are herein incorporated by reference in their entirety. In one embodiment, the antibody may have a sequence as shown by SEQ ID NO: 1, 2, 17, 18, 33, 34, 49, 50, 65, 66, 81, 82, 9-16, 25-32, 41-48, 57-64, 73-80, 89-96 of WO2016094566.

In some embodiments, the antibodies (or fragments thereof) encoded by the viral genomes of the present invention may be any of those taught in Nos. U.S. Pat. Nos. 8,778,343 and 9,125,846, International Publications WO2012051498 and WO2011026031, or United States Publication Nos. US20150004169 and US20150322143, the contents of each of which are herein incorporated by reference in their entirety. Such antibodies may include those that bind to oligomeric species of tau. Further, such an antibody may be referred to as TOMA (tau oligomer monoclonal antibody), as described in Castillo-Carranza et at (Castillo-Carranza, D L et al., 2014 J Neurosci 34(12)4260-72) the contents of which are herein incorporated by reference in their entirety. In one embodiment, the antibody that binds oligomeric tau may be TTC-99.

In some embodiments, the antibodies (or fragments thereof) encoded by the viral genomes of the present invention may be any of those taught in International Publications WO2014059442, the contents of which are herein incorporated by reference in their entirety. Such antibodies may include those that bind to oligomeric species of tau.

In some embodiments, the antibodies (or fragments thereof) encoded by the viral genomes of the present invention may be any of those taught in the International Publications WO2014008404 and WO2016126993, United States Patent Publication US20150183855, Yanamandra, K et al., 2013 Neuron 80(2):402-14 and Yanamandra, K et al 2015 Ann Clin Transl Neurol 2(3):278-88, the contents of each of which are herein incorporated by reference in their entirety. Such antibodies may block tau seeding. Non-limiting examples of antibodies described in these publications include HJ8.1.1, HJ81.2, HJ8.2, HJ8.3, HJ8.4, HJ8.5, HJ8.7, HJ8.8, HJ9.1, HJ9.2, HJ9.3, HJ9.4, HJ95, and variants thereof. Non-limiting examples of targeted epitopes of tau may include amino acids 22-34, 385-391, 405-411, 3-6, 118-122, 386-401, 7-13, and/or 272-281 of human tau.

In some embodiments, the antibodies (or fragments thereof) encoded by the viral genomes of the present invention may be any of those taught in the International Publications WO2002062851, the contents of which are herein incorporated by reference in their entirety.

In some embodiments, the antibodies (or fragments thereof) encoded by the viral genomes of the present invention may be as described in Bright, J et al., 2015 Neurobiol of Aging 36:693.709; Pedersen, J T and Sigurdsson E M, 2015 Trends Mol Med 21(6):394-402; Levites, Y et al 2015 J Neurosci 35(16)6265-76; Jicha et al 1999 J Neurosci 19(17):7486-94; Reyes J F et al., 2012 Acta Neuropathol 123(1):119-32; Reynolds M R, et al, 2006 J Neurosci 26(42):10636-45; Gamblin, T C et al 2003 PNAS 100(17):10032.7; Castillo-Carranza, D L et al., 2014 J Neurosci 34(12)4260-72; Walls, K C et al., 2014 Neurosci Lett 575:96-100; Yanamandra, K et al., 2013 Neuron 80(2):402-14; Yanamandra, K et al 2015 Ann Clin Transl Neurol 2(3):278.88; Allen B, et al., 2002 J Neurosci 22(21):9340.51; Gotz, J et al., 2010 Biochem Biophys Acta 1802(10):860-71; Hasegawa, M et al 1996 FEBS Lett 384:25-30; Carmel, G et al 1996 J Biol Chem 271(51):32780-32795; Jicha, G A et al, 1997 J Neurosci Res 48(2):128-132; Jicha, G A et al., 1997 J Neurochem 69(5):2087-95; the contents of each of which are herein incorporated by reference in their entirety.

Anti-tau antibodies or fragments thereof encoded by the viral genomes of the present invention may be any commercially available anti-tau antibody known in the art or developed by a person with skill in the art. Non-limiting examples of commercially available anti-tau antibodies include EPR2396(2) (pThr⁵⁰; Abcam, Cambridge, Mass.), 5H911 (pThr¹⁸¹; ThermoFisher, Waltham, Mass.), M7004D06 (pThr¹⁸¹; BioLegend, San Diego, Calif.), 1E7 (pThr¹⁸¹; EMD Millipore, Billerica, Mass.), EPR2400 (pSer¹⁹⁸; Abcam, Cambridge, Mass.), EPR2401Y (pSer¹⁹⁹; Abcam, Cambridge, Mass.), 2H23L4 (pSer¹⁹⁹; ThermoFisher, Waltham, Mass.), EPR2402 (pSer²⁰²; Abcam, Cambridge, Mass.), 10F8 (pSer²⁰²; Abcam, Cambridge, Mass.), EPR2403(2) (pThr²⁰⁵; Abcam, Cambridge, Mass.), EPR1884(2) (pSer²¹⁴; Abcam, Cambridge, Mass.), EPR2488 (pThr²³¹; Abcam, Cambridge, Mass.), 1H6L6 (pThr²³¹; ThermoFisher, Waltham, Mass.), 3G3 (pThr²³¹, pSer²³⁵; Abcam, Cambridge, Mass.), EPR2452 (pSer²³⁵; Abcam, Cambridge, Mass.), 12G10 (pSer²³⁸; Abcam, Cambridge, Mass.), EPR2454 (pSer²⁶²; Abcam, Cambridge, Mass.), EPR2457(2) (pSer³²⁴; Abcam, Cambridge, Mass.), EPR2603 (pSer³⁵⁶; Abcam, Cambridge, Mass.), EPR2731 (pSer³⁹⁶; Abcam, Cambridge, Mass.), EPR2605 (pSer⁴⁰⁴; Abcam, Cambridge, Mass.), EPR2866 (pSer⁴²²; Abcam, Cambridge, Mass.), 1A4 (pTau¹⁷⁷⁻¹⁸⁷; Origene, Rockville, Md.), 7G9 (pTau¹⁷⁷⁻¹⁸⁷; Origene, Rockville, Md.), 9B4 (pTau¹⁷⁷⁻¹⁸⁷; Origene, Rockville, Md.), 2A4 (pTau¹⁷⁷⁻¹⁸⁷; Origene, Rockville, Md.), 9G3 (pTyr¹⁸; NovusBio, Littleton, Colo.), EPR2455(2) (pSer⁶¹⁰; Abcam, Cambridge, Mass.), EP2456Y (pSer⁶²²; Abcam, Cambridge, Mass.; EMD Millipore, Billerica, Mass.), SMI 51 (PHF Tau⁹⁵⁻¹⁰⁸; BioLegend, San Diego, Calif.), TOMA-1 (Oligomeric Tau; EMD Millipore, Billerica, Mass.), Tau-nY18 (nTyr¹³; Origene, Rockville, Md.; BioLegend, San Diego, Calif.; EMD Millipore, Billerica, Mass.), Tau-nY29 (nTyr²⁹; BioLegend, San Diego, Calif.; EMD Millipore, Billerica, Mass.; Abcam, Cambridge, Mass.), 1C9.G6 (di-methyl-Lys²⁸¹; BioLegend, San Diego, Calif.), 7G5.F4 (di-methyl-Lys³¹¹; BioLegend, San Diego, Calif.), TNT-1 (Tau²⁻¹⁸; EMD Millipore, Billerica, Mass.), TNT-2 (Tau²⁻¹⁸; EMD Millipore, Billerica, Mass.), 7B8 (Tau⁵⁻¹²; Abcam, Cambridge, Mass.), Tau-13 (Tau²⁰⁻³⁵; BioLegend, San Diego, Calif.), 1-100 (Tau¹⁻¹⁰⁰; BioLegend, San Diego, Calif.), 2G9.F10 (Tau¹⁵⁷⁻¹⁶⁸; BioLegend, San Diego, Calif.; Origene, Rockville, Md.), 39E10 (Tau¹⁸⁹⁻¹⁹⁵; BioLegend, San Diego, Calif.; Origene, Rockville, Md.), 77E9 (Tau¹⁸⁵⁻¹⁹⁵; BioLegend, San Diego, Calif.; Origene, Rockville, Md.), ATS (pSer²⁰², pSer²⁰⁵; ThermoFisher, Waltham, Mass.), AT100 (pSer²¹², pSer²¹⁴; ThermoFisher, Waltham, Mass.), PHF-6 (pThr²³¹; NovusBio, Littleton, Colo.; EMD Millipore, Billerica, Mass.; BioLegend, San Diego, Calif.; ThermoFisher, Waltham, Mass.), AT180 (pThr²³¹; ThermoFisher, Waltham, Mass.), AT270 (pThr¹⁸¹; ThermoFisher, Waltham, Mass.), PHF-13 (pSer³⁹⁶; ThermoFisher, Waltham, Mass.; BioLegend, San Diego, Calif.), TauC3 (Asp⁴²¹; BioLegend, San Diego, Calif.; EMD Millipore, Billerica, Mass.; ThermoFisher, Waltham, Mass.), Tau12 (Tau⁶⁻¹⁸; BioLegend, San Diego, Calif.; EMD Millipore, Billerica, Mass.), Tau5 (Tau²¹⁰⁻²⁴¹; BioLegend, San Diego, Calif.; EMD Millipore, Billerica, Mass.; Abcam, Cambridge Mass.; ThermoFisher, Waltham, Mass.), HT7 (Tau¹⁵⁹⁻¹⁶³; ThermoFisher, Waltham, Mass.), 77G7 (Tau³¹⁶⁻³⁵⁵; BioLegend, San Diego, Calif.), Tau46 (Tau⁴⁰⁴⁻⁴⁴¹; BioLegend, San Diego, Calif.; NovusBio, Littleton, Colo.; Abcam, Cambridge, Mass.), UMAB239 (Tau⁶²³⁻⁷⁵⁸; Origene, Rockville, Md.), OTI6G3 (Tau⁶²³⁻⁷⁵⁸; Origene, Rockville, Md.), OTI13E11 (Tau⁶²³⁻⁷⁵⁸; Origene, Rockville, Md.), OTI13B5 (Tau⁶²³⁻⁷⁵⁸; Origene, Rockville, Md.), E178 (Tau⁷⁰⁰⁻⁸⁰⁰; Abcam, Cambridge, Mass.), SP70 (N-terminal Tau; Origene, Rockville, Md.; NovusBio, Littleton, Colo.; ThermoFisher, Waltham, Mass.; Abcam, Cambridge, Mass.), C45 (N-terminal Tau; Origene, Rockville, Md.), Tau7 (C-terminal Tau; EMD Millipore, Billerica, Mass.), S.125.0 (C-terminal Tau; ThermoFisher, Waltham, Mass.), 8E6/C11 (Three-repeat Tau²⁰⁹⁻²²⁴; EMD Millipore, Billerica, Mass.), 1E1/A6 (Four-repeat Tau²⁷⁵⁻²⁹¹; EMD Millipore, Billerica, Mass.), 7D12.1 (Four-repeat Tau²⁷⁵⁻²⁹¹; EMD Millipore, Billerica, Mass.), 5C7 (Four-repeat Tau²⁶⁷⁻²⁷⁸; BioLegend, San Diego, Calif.; Origene, Rockville, Md.), 5F9 (Four-repeat Tau²⁷⁵⁻²⁹¹; BioLegend, San Diego, Calif.; Origene, Rockville, Md.), 3H6.H7 (ON Tau³⁹⁻⁵⁰; BioLegend, San Diego, Calif.; Origene, Rockville, Md.), 4H5.B9 (1N Tau⁶⁸⁻⁷⁹; BioLegend, San Diego, Calif.; Origene, Rockville, Md.), 71C11 (2N Tau; BioLegend, San Diego, Calif.), PC1C6 (unphosphorylated tau; EMD Millipore, Billerica, Mass.), Tau2 (BioLegend, San Diego, Calif.; Origene, Rockville, Md.; EMD Millipore, Billerica, Mass.), 2E9 (Origene, Rockville, Md.; NovusBio, Littleton, Colo.), 4F1 (Origene, Rockville, Md.; NovusBio, Littleton, Colo.), 5B10 (NovusBio, Littleton, Colo.); 5E2 (EMD Millipore, Billerica, Mass.), Tau-93 (Origene, Rockville, Md.), T14 (ThermoFisher, Waltham, Mass.), T46 (ThermoFisher, Waltham, Mass.), BT2 (ThermoFisher, Waltham, Mass.) and/or variants or derivates thereof.

In one embodiment, the antibodies encoded by the viral genomes of the present invention may be multispecific antibodies for transferrin receptor and a brain antigen, wherein the brain antigen may be tau, as described in International Publication WO2016081643, the contents of which are herein incorporated by reference in their entirety. In one embodiment, the antibody may have a sequence as given by SEQ ID NO: 160 or 161 of WO2016081643.

In one embodiment, the antibodies encoded by the viral genomes of the present invention are any of those described in U.S. Pat. Nos. 8,871,447, 8,420,613, International Publication No. WO2014193935, WO2010011999, or in United States Publication Nos. US20110250217, US20110020237, US20100316590, or US20120225864, the contents of each of which are herein incorporated by reference in their entirety. In one embodiment, the antibody recognizes an amyloidogenic or aggregating protein.

### Antibodies for the Treatment of Migraine or Pain

In some embodiments, the payload region of the AAV particle comprises one or more nucleic acid sequences encoding migraine-associated antibodies, variants or fragments thereof.

In some embodiments, the payload region of the AAV particle comprises one or more nucleic acid sequences encoding one or more of the payload antibody polypeptides listed in Table 12, or variants or fragments thereof. As used herein, “antibody polynucleotide” refers to a nucleic acid sequence encoding an antibody polypeptide.

In some embodiments, the payload region of the AAV particle comprises one or more nucleic acid sequences listed in Table 12, or variants or fragments thereof.

In some embodiments, the payload region of the AAV particle comprises a nucleic acid sequence encoding a payload antibody with at least 50% identity to one or more payload antibody polypeptides listed in Table 12. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%,95%, 96%,97%,98%, 99%, or 100% identity to one or more of the payload antibody polypeptides listed in Table 12, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 12, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 12, or variants or fragments thereof.

In some embodiments, the heavy chain of the encoded antibody polypeptide may have 50%,51%,52%, 53%,54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 12, or variants or fragments thereof.

In some embodiments, the light chain of the encoded antibody polypeptide may have 50%, 51%, 52%,53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86% 87% 88%, 89%, 90%, 91%, 92%, 93%, 94%, 95%, 96%, 97%, 98%, 99%, or 100% identity to one or more of the payload light chain antibody polypeptides listed in Table 12, or variants or fragments thereof.

In some embodiments, the CDR region of the encoded antibody polypeptide may have 50%,51%,52%, 53%,54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 12, or variants or fragments thereof.

In some embodiments, the payload antibody has 90% identity to one or more of the antibody polypeptides listed in Table 12, or variants or fragments thereof.

In some embodiments, the payload antibody has 91% identity to one or more of the antibody polypeptides listed in Table 12, or variants or fragments thereof.

In some embodiments, the payload antibody has 92% identity to one or more of the antibody polypeptides listed in Table 12, or variants or fragments thereof.

In some embodiments, the payload antibody has 93% identity to one or more of the antibody polypeptides listed in Table 12, or variants or fragments thereof.

In some embodiments, the payload antibody has 94% identity to one or more of the antibody polypeptides listed in Table 12, or variants or fragments thereof.

In some embodiments, the payload antibody has 95% identity to one or more of the antibody polypeptides listed in Table 12, or variants or fragments thereof.

In some embodiments, the payload antibody has 96% identity to one or more of the antibody polypeptides listed in Table 12, or variants or fragments thereof.

In some embodiments, the payload antibody has 97% identity to one or more of the antibody polypeptides listed in Table 12, or variants or fragments thereof.

In some embodiments, the payload antibody has 98% identity to one or more of the antibody polypeptides listed in Table 12, or variants or fragments thereof.

In some embodiments, the payload antibody has 99% identity to one or more of the antibody polypeptides listed in Table 12, or variants or fragments thereof.

In some embodiments, the payload antibody has 100% identity to one or more of the antibody polypeptides listed in Table 12, or variants or fragments thereof.

In some embodiments, the payload region of the AAV particle comprises a nucleic acid sequence with at least 50% identity to one or more nucleic acid sequences listed in Table 12, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 12, or variants or fragments thereof.

In some embodiments, the payload nucleic acid sequence has 90% identity to one or more of the nucleic acid sequences listed in Table 12, or variants or fragments thereof.

In some embodiments, the payload nucleic acid sequence has 91% identity to one or more of the nucleic acid sequences listed in Table 12, or variants or fragments thereof.

In some embodiments, the payload nucleic acid sequence has 92% identity to one or more of the nucleic acid sequences listed in Table 12, or variants or fragments thereof.

In some embodiments, the payload nucleic acid sequence has 93% identity to one or more of the nucleic acid sequences listed in Table 12, or variants or fragments thereof.

In some embodiments, the payload nucleic acid sequence has 94% identity to one or more of the nucleic acid sequences listed in Table 12, or variants or fragments thereof.

In some embodiments, the payload nucleic acid sequence has 95% identity to one or more of the nucleic acid sequences listed in Table 12, or variants or fragments thereof.

In some embodiments, the payload nucleic acid sequence has 96% identity to one or more of the nucleic acid sequences listed in Table 12, or variants or fragments thereof.

In some embodiments, the payload nucleic acid sequence has 97% identity to one or more of the nucleic acid sequences listed in Table 12, or variants or fragments thereof.

In some embodiments, the payload nucleic acid sequence has 98% identity to one or more of the nucleic acid sequences listed in Table 12, or variants or fragments thereof.

In some embodiments, the payload nucleic acid sequence has 99% identity to one or more of the nucleic acid sequences listed in Table 12, or variants or fragments thereof.

In some embodiments, the payload nucleic acid sequence has 100% identity to one or more of the nucleic acid sequences listed in Table 12, or variants or fragments thereof.

In some embodiments, the payload antibody may be variants of any of the antibody polypeptides listed in Table 12, that exclude one or more amino acids designated as “X”, or “x” in the described polypeptide sequence, wherein X may represent any amino acid. In some embodiments, the payload nucleic acid sequence may be variants of any of the nucleic acid sequences listed in Table 12, that exclude one or more nucleic acids designated as “n” or “N” in the described nucleic acid sequence, wherein n may represent any nucleic acid.

TABLE 12
Migraine antibodies

| | | Type | | SEQ |
|---|---|---|---|---|
| Ab ID | Component | (PRT/DNA) | Reference | ID NO. |
| MGR1 | CDR | DNA | U.S. Pat. No. 9,745,373; SEQ ID NO: 208 | 8175 |
| MGR2 | CDR | DNA | U.S. Pat. No. 9,745,373; SEQ ID NO: 258 | 8176 |
| MGR3 | CDR | DNA | U.S. Pat. No. 9,745,373; SEQ ID NO: 248 | 8177 |
| MGR4 | CDR | DNA | U.S. Pat. No. 9,745,373; SEQ ID NO: 268 | 8178 |
| MGR5 | CDR | DNA | U.S. Pat. No. 9,745,373; SEQ ID NO: 148 | 8179 |
| MGR6 | CDR | DNA | U.S. Pat. No. 9,745,373; SEQ ID NO: 228 | 8180 |
| MGR7 | CDR | DNA | U.S. Pat. No. 9,745,373; SEQ ID NO: 251 | 8181 |
| MGR8 | CDR | DNA | U.S. Pat. No. 9,745,373; SEQ ID NO: 252 | 8182 |
| MGR9 | CDR | DNA | U.S. Pat. No. 9,745,373; SEQ ID NO: 235 | 8183 |
| MGR10 | CDR | DNA | U.S. Pat. No. 9,745,373; SEQ ID NO: 225 | 8184 |
| MGR11 | CDR | DNA | U.S. Pat. No. 9,745,373; SEQ ID NO: 215 | 8185 |
| MGR12 | CDR | DNA | U.S. Pat. No. 9,745,373; SEQ ID NO: 205 | 8186 |
| MGR13 | CDR | DNA | U.S. Pat. No. 9,745,373; SEQ ID NO: 175 | 8187 |
| MGR14 | CDR | DNA | U.S. Pat. No. 9,745,373; SEQ ID NO: 145 | 8188 |
| MGR15 | CDR | DNA | U.S. Pat. No. 9,745,373; SEQ ID NO: 265 | 8189 |
| MGR16 | CDR | DNA | U.S. Pat. No. 9,745,373; SEQ ID NO: 143 | 8190 |
| MGR17 | CDR | DNA | U.S. Pat. No. 9,745,373; SEQ ID NO: 255 | 8191 |
| MGR18 | CDR | DNA | U.S. Pat. No. 9,745,373; SEQ ID NO: 245 | 8192 |
| MGR19 | CDR | DNA | U.S. Pat. No. 9,745,373; SEQ ID NO: 207 | 8193 |
| MGR20 | CDR | DNA | U.S. Pat. No. 9,745,373; SEQ ID NO: 247 | 8194 |
| MGR21 | CDR | DNA | U.S. Pat. No. 9,745,373; SEQ ID NO: 147 | 8195 |
| MGR22 | CDR | DNA | U.S. Pat. No. 9,745,373; SEQ ID NO: 227 | 8196 |
| MGR23 | CDR | DNA | U.S. Pat. No. 9,745,373; SEQ ID NO: 177 | 8197 |
| MGR24 | CDR | DNA | U.S. Pat. No. 9,745,373; SEQ ID NO: 257 | 8198 |
| MGR25 | CDR | DNA | U.S. Pat. No. 9,745,373; SEQ ID NO: 217 | 8199 |
| MGR26 | CDR | DNA | U.S. Pat. No. 9,745,373; SEQ ID NO: 237 | 8200 |
| MGR27 | CDR | DNA | U.S. Pat. No. 9,745,373; SEQ ID NO: 213 | 8201 |
| MGR28 | CDR | DNA | U.S. Pat. No. 9,745,373; SEQ ID NO: 214 | 8202 |
| MGR29 | CDR | DNA | U.S. Pat. No. 9,745,373; SEQ ID NO: 233 | 8203 |
| MGR30 | CDR | DNA | U.S. Pat. No. 9,745,373; SEQ ID NO: 270 | 8204 |
| MGR31 | CDR | DNA | U.S. Pat. No. 9,745,373; SEQ ID NO: 266 | 8205 |
| MGR32 | CDR | DNA | U.S. Pat. No. 9,745,373; SEQ ID NO: 186 | 8206 |
| MGR33 | CDR | DNA | U.S. Pat. No. 9,745,373; SEQ ID NO: 176 | 8207 |
| MGR34 | CDR | DNA | U.S. Pat. No. 9,745,373; SEQ ID NO: 267 | 8208 |
| MGR35 | CDR | DNA | U.S. Pat. No. 9,745,373; SEQ ID NO: 178 | 8209 |
| MGR36 | CDR | DNA | U.S. Pat. No. 9,745,373; SEQ ID NO: 150 | 8210 |
| MGR37 | CDR | DNA | U.S. Pat. No. 9,745,373; SEQ ID NO: 229 | 8211 |
| MGR38 | CDR | DNA | U.S. Pat. No. 9,745,373; SEQ ID NO: 159 | 8212 |
| MGR39 | CDR | DNA | U.S. Pat. No. 9,745,373; SEQ ID NO: 149 | 8213 |
| MGR40 | CDR | DNA | U.S. Pat. No. 9,745,373; SEQ ID NO: 179 | 8214 |
| MGR41 | CDR | DNA | U.S. Pat. No. 9,745,373; SEQ ID NO: 249 | 8215 |
| MGR42 | CDR | DNA | U.S. Pat. No. 9,745,373; SEQ ID NO: 219 | 8216 |
| MGR43 | CDR | DNA | U.S. Pat. No. 9,745,373; SEQ ID NO: 209 | 8217 |
| MGR44 | CDR | DNA | U.S. Pat. No. 9,745,373; SEQ ID NO: 146 | 8218 |
| MGR45 | CDR | DNA | U.S. Pat. No. 9,745,373; SEQ ID NO: 226 | 8219 |
| MGR46 | CDR | DNA | U.S. Pat. No. 9,745,373; SEQ ID NO: 269 | 8220 |
| MGR47 | CDR | PRT | WO2007076336; SEQ ID NO: 6 | 8221 |
| MGR48 | CDR | PRT | WO2007076336; SEQ ID NO: 8 | 8222 |
| MGR49 | CDR | PRT | WO2007076336; SEQ ID NO: 11 | 8223 |
| MGR50 | CDR | PRT | WO2007076336; SEQ ID NO: 21 | 8224 |
| MGR51 | CDR | PRT | WO2007076336; SEQ ID NO: 16 | 8225 |
| MGR52 | CDR | PRT | US20150322142; SEQ ID NO: 4 | 8226 |
| MGR53 | CDR | PRT | US20150322142; SEQ ID NO: 55 | 8227 |
| MGR54 | CDR | PRT | US20150322142; SEQ ID NO: 7 | 8228 |
| MGR55 | CDR | PRT | US20150322142; SEQ ID NO: 3 | 8229 |
| MGR56 | CDR | PRT | WO2007076336; SEQ ID NO: 24 | 8230 |
| MGR57 | CDR | PRT | WO2007076336; SEQ ID NO: 22 | 8231 |
| MGR58 | CDR | PRT | WO2007076336; SEQ ID NO: 17 | 8232 |
| MGR59 | CDR | PRT | WO2007076336; SEQ ID NO: 14 | 8233 |
| MGR60 | CDR | PRT | US20150322142; SEQ ID NO: 6 | 8234 |
| MGR61 | CDR | PRT | US20150322142; SEQ ID NO: 53 | 8235 |
| MGR62 | CDR | PRT | WO2007076336; SEQ ID NO: 5 | 8236 |
| MGR63 | CDR | PRT | WO2007076336; SEQ ID NO: 20 | 8237 |
| MGR64 | CDR | PRT | WO2007076336; SEQ ID NO: 19 | 8238 |
| MGR65 | CDR | PRT | WO2007076336; SEQ ID NO: 15 | 8239 |
| MGR66 | CDR | PRT | WO2007076336; SEQ ID NO: 13 | 8240 |
| MGR67 | CDR | PRT | WO2007076336; SEQ ID NO: 10 | 8241 |
| MGR68 | CDR | PRT | WO2007076336; SEQ ID NO: 12 | 8242 |
| MGR69 | CDR | PRT | US20150322142; SEQ ID NO: 8 | 8243 |
| MGR70 | CDR | PRT | US20150322142; SEQ ID NO: 54 | 8244 |
| MGR71 | CDR | PRT | US20150322142; SEQ ID NO: 5 | 8245 |
| MGR72 | CDR | PRT | WO2007076336; SEQ ID NO: 23 | 8246 |
| MGR73 | CDR | PRT | WO2007076336; SEQ ID NO: 7 | 8247 |
| MGR74 | CDR | PRT | US20120294802; SEQ ID NO: 56 | 8248 |
| MGR75 | CDR | PRT | US20120294802; SEQ ID NO: 58 | 8249 |
| MGR76 | CDR | PRT | US20120294802; SEQ ID NO: 59 | 8250 |
| MGR77 | CDR | PRT | US20120294802; SEQ ID NO: 57 | 8251 |
| MGR78 | CDR | PRT | US20120294802; SEQ ID NO: 55 | 8252 |
| MGR79 | CDR | PRT | U.S. Pat. No. 9,102,731; SEQ ID NO: 64 | 8253 |
| MGR80 | CDR | PRT | U.S. Pat. No. 9,102,731; SEQ ID NO: 49 | 8254 |
| MGR81 | CDR | PRT | U.S. Pat. No. 9,102,731; SEQ ID NO: 47 | 8255 |
| MGR82 | CDR | PRT | U.S. Pat. No. 9,102,731; SEQ ID NO: 102 | 8256 |
| MGR83 | CDR | PRT | U.S. Pat. No. 9,102,731; SEQ ID NO: 80 | 8257 |
| MGR84 | CDR | PRT | U.S. Pat. No. 9,102,731; SEQ ID NO: 43 | 8258 |
| MGR85 | CDR | PRT | U.S. Pat. No. 9,102,731; SEQ ID NO: 84 | 8259 |
| MGR86 | CDR | PRT | U.S. Pat. No. 9,102,731; SEQ ID NO: 81 | 8260 |
| MGR87 | CDR | PRT | U.S. Pat. No. 9,102,731; SEQ ID NO: 96 | 8261 |
| MGR88 | CDR | PRT | U.S. Pat. No. 9,102,731; SEQ ID NO: 75 | 8262 |
| MGR89 | CDR | PRT | U.S. Pat. No. 9,102,731; SEQ ID NO: 78 | 8263 |
| MGR90 | CDR | PRT | U.S. Pat. No. 9,102,731; SEQ ID NO: 88 | 8264 |
| MGR91 | CDR | PRT | U.S. Pat. No. 9,102,731; SEQ ID NO: 92 | 8265 |
| MGR92 | CDR | PRT | U.S. Pat. No. 9,102,731; SEQ ID NO: 99 | 8266 |
| MGR93 | CDR | PRT | U.S. Pat. No. 9,102,731; SEQ ID NO: 87 | 8267 |
| MGR94 | CDR | PRT | U.S. Pat. No. 9,102,731; SEQ ID NO: 55 | 8268 |
| MGR95 | CDR | PRT | U.S. Pat. No. 9,102,731; SEQ ID NO: 89 | 8269 |
| MGR96 | CDR | PRT | U.S. Pat. No. 9,102,731; SEQ ID NO: 67 | 8270 |
| MGR97 | CDR | PRT | U.S. Pat. No. 9,102,731; SEQ ID NO: 70 | 8271 |
| MGR98 | CDR | PRT | U.S. Pat. No. 9,102,731; SEQ ID NO: 94 | 8272 |
| MGR99 | CDR | PRT | U.S. Pat. No. 9,102,731; SEQ ID NO: 52 | 8273 |
| MGR100 | CDR | PRT | U.S. Pat. No. 9,102,731; SEQ ID NO: 90 | 8274 |
| MGR101 | CDR | PRT | U.S. Pat. No. 9,102,731; SEQ ID NO: 44 | 8275 |
| MGR102 | CDR | PRT | U.S. Pat. No. 9,102,731; SEQ ID NO: 82 | 8276 |
| MGR103 | CDR | PRT | U.S. Pat. No. 9,102,731; SEQ ID NO: 54 | 8277 |
| MGR104 | CDR | PRT | U.S. Pat. No. 9,102,731; SEQ ID NO: 60 | 8278 |
| MGR105 | CDR | PRT | U.S. Pat. No. 9,102,731; SEQ ID NO: 65 | 8279 |
| MGR106 | CDR | PRT | U.S. Pat. No. 9,102,731; SEQ ID NO: 110 | 8280 |
| MGR107 | CDR | PRT | U.S. Pat. No. 9,102,731; SEQ ID NO: 58 | 8281 |
| MGR108 | CDR | PRT | U.S. Pat. No. 9,102,731; SEQ ID NO: 50 | 8282 |
| MGR109 | CDR | PRT | U.S. Pat. No. 9,102,731; SEQ ID NO: 68 | 8283 |
| MGR110 | CDR | PRT | U.S. Pat. No. 9,102,731; SEQ ID NO: 105 | 8284 |
| MGR111 | CDR | PRT | U.S. Pat. No. 9,102,731; SEQ ID NO: 59 | 8285 |
| MGR112 | CDR | PRT | U.S. Pat. No. 9,102,731; SEQ ID NO: 56 | 8286 |
| MGR113 | CDR | PRT | U.S. Pat. No. 9,102,731; SEQ ID NO: 113 | 8287 |
| MGR114 | CDR | PRT | U.S. Pat. No. 9,102,731; SEQ ID NO: 76 | 8288 |
| MGR115 | CDR | PRT | U.S. Pat. No. 9,102,731; SEQ ID NO: 53 | 8289 |
| MGR116 | CDR | PRT | U.S. Pat. No. 9,102,731; SEQ ID NO: 51 | 8290 |
| MGR117 | CDR | PRT | U.S. Pat. No. 9,102,731; SEQ ID NO: 71 | 8291 |
| MGR118 | CDR | PRT | U.S. Pat. No. 9,102,731; SEQ ID NO: 72 | 8292 |
| MGR119 | CDR | PRT | U.S. Pat. No. 9,102,731; SEQ ID NO: 106 | 8293 |
| MGR120 | CDR | PRT | U.S. Pat. No. 9,102,731; SEQ ID NO: 66 | 8294 |
| MGR121 | CDR | PRT | U.S. Pat. No. 9,102,731; SEQ ID NO: 48 | 8295 |
| MGR122 | CDR | PRT | U.S. Pat. No. 9,102,731; SEQ ID NO: 103 | 8296 |
| MGR123 | CDR | PRT | U.S. Pat. No. 9,102,731; SEQ ID NO: 69 | 8297 |
| MGR124 | CDR | PRT | U.S. Pat. No. 9,102,731; SEQ ID NO: 107 | 8298 |
| MGR125 | CDR | PRT | U.S. Pat. No. 9,102,731; SEQ ID NO: 95 | 8299 |
| MGR126 | CDR | PRT | U.S. Pat. No. 9,102,731; SEQ ID NO: 91 | 8300 |
| MGR127 | CDR | PRT | U.S. Pat. No. 9,102,731; SEQ ID NO: 77 | 8301 |
| MGR128 | CDR | PRT | U.S. Pat. No. 9,102,731; SEQ ID NO: 124 | 8302 |
| MGR129 | CDR | PRT | U.S. Pat. No. 9,102,731; SEQ ID NO: 61 | 8303 |
| MGR130 | CDR | PRT | U.S. Pat. No. 9,102,731; SEQ ID NO: 46 | 8304 |
| MGR131 | CDR | PRT | U.S. Pat. No. 9,102,731; SEQ ID NO: 57 | 8305 |
| MGR132 | CDR | PRT | U.S. Pat. No. 9,102,731; SEQ ID NO: 114 | 8306 |
| MGR133 | CDR | PRT | U.S. Pat. No. 9,102,731; SEQ ID NO: 73 | 8307 |
| MGR134 | CDR | PRT | U.S. Pat. No. 9,102,731; SEQ ID NO: 42 | 8308 |
| MGR135 | CDR | PRT | U.S. Pat. No. 9,102,731; SEQ ID NO: 62 | 8309 |
| MGR136 | CDR | PRT | U.S. Pat. No. 9,102,731; SEQ ID NO: 45 | 8310 |
| MGR137 | CDR | PRT | U.S. Pat. No. 9,102,731; SEQ ID NO: 115 | 8311 |
| MGR138 | CDR | PRT | U.S. Pat. No. 9,102,731; SEQ ID NO: 98 | 8312 |
| MGR139 | CDR | PRT | U.S. Pat. No. 9,102,731; SEQ ID NO: 128 | 8313 |
| MGR140 | CDR | PRT | U.S. Pat. No. 9,102,731; SEQ ID NO: 79 | 8314 |
| MGR141 | CDR | PRT | U.S. Pat. No. 9,102,731; SEQ ID NO: 100 | 8315 |
| MGR142 | CDR | PRT | U.S. Pat. No. 9,102,731; SEQ ID NO: 63 | 8316 |
| MGR143 | CDR | PRT | U.S. Pat. No. 9,102,731; SEQ ID NO: 97 | 8317 |
| MGR144 | CDR | PRT | U.S. Pat. No. 9,102,731; SEQ ID NO: 74 | 8318 |
| MGR145 | CDR | PRT | U.S. Pat. No. 9,102,731; SEQ ID NO: 129 | 8319 |
| MGR146 | CDR | PRT | U.S. Pat. No. 9,102,731; SEQ ID NO: 86 | 8320 |
| MGR147 | CDR | PRT | U.S. Pat. No. 9,102,731; SEQ ID NO: 101 | 8321 |
| MGR148 | CDR | PRT | U.S. Pat. No. 9,102,731; SEQ ID NO: 83 | 8322 |
| MGR149 | CDR | PRT | U.S. Pat. No. 9,102,731; SEQ ID NO: 93 | 8323 |
| MGR150 | CDR | PRT | U.S. Pat. No. 9,102,731; SEQ ID NO: 122 | 8324 |
| MGR151 | CDR | PRT | U.S. Pat. No. 9,102,731; SEQ ID NO: 104 | 8325 |
| MGR152 | CDR | PRT | U.S. Pat. No. 9,102,731; SEQ ID NO: 108 | 8326 |
| MGR153 | CDR | PRT | U.S. Pat. No. 9,102,731; SEQ ID NO: 118 | 8327 |
| MGR154 | CDR | PRT | U.S. Pat. No. 9,102,731; SEQ ID NO: 126 | 8328 |
| MGR155 | CDR | PRT | U.S. Pat. No. 9,102,731; SEQ ID NO: 132 | 8329 |
| MGR156 | CDR | PRT | U.S. Pat. No. 9,102,731; SEQ ID NO: 135 | 8330 |
| MGR157 | CDR | PRT | U.S. Pat. No. 9,102,731; SEQ ID NO: 109 | 8331 |
| MGR158 | CDR | PRT | U.S. Pat. No. 9,102,731; SEQ ID NO: 130 | 8332 |
| MGR159 | CDR | PRT | U.S. Pat. No. 9,102,731; SEQ ID NO: 111 | 8333 |
| MGR160 | CDR | PRT | U.S. Pat. No. 9,102,731; SEQ ID NO: 116 | 8334 |
| MGR161 | CDR | PRT | U.S. Pat. No. 9,102,731; SEQ ID NO: 112 | 8335 |
| MGR162 | CDR | PRT | U.S. Pat. No. 9,102,731; SEQ ID NO: 117 | 8336 |
| MGR163 | CDR | PRT | U.S. Pat. No. 9,102,731; SEQ ID NO: 120 | 8337 |
| MGR164 | CDR | PRT | U.S. Pat. No. 9,102,731; SEQ ID NO: 131 | 8338 |
| MGR165 | CDR | PRT | U.S. Pat. No. 9,102,731; SEQ ID NO: 123 | 8339 |
| MGR166 | CDR | PRT | U.S. Pat. No. 9,102,731; SEQ ID NO: 134 | 8340 |
| MGR167 | CDR | PRT | U.S. Pat. No. 9,102,731; SEQ ID NO: 127 | 8341 |
| MGR168 | CDR | PRT | U.S. Pat. No. 9,102,731; SEQ ID NO: 133 | 8342 |
| MGR169 | CDR | PRT | U.S. Pat. No. 9,102,731; SEQ ID NO: 136 | 8343 |
| MGR170 | CDR | PRT | U.S. Pat. No. 9,102,731; SEQ ID NO: 125 | 8344 |
| MGR171 | CDR | PRT | U.S. Pat. No. 9,102,731; SEQ ID NO: 121 | 8345 |
| MGR172 | CDR | PRT | U.S. Pat. No. 9,896,502; SEQ ID NO: 4 | 8346 |
| MGR173 | CDR | PRT | U.S. Pat. No. 9,896,502; SEQ ID NO: 7 | 8347 |
| MGR174 | CDR | PRT | U.S. Pat. No. 9,896,502; SEQ ID NO: 3 | 8348 |
| MGR175 | CDR | PRT | U.S. Pat. No. 9,896,502; SEQ ID NO: 6 | 8349 |
| MGR176 | CDR | PRT | U.S. Pat. No. 9,896,502; SEQ ID NO: 8 | 8350 |
| MGR177 | CDR | PRT | U.S. Pat. No. 9,896,502; SEQ ID NO: 5 | 8351 |
| MGR178 | CDR | PRT | U.S. Pat. No. 9,745,373; SEQ ID NO: 129 | 8352 |
| MGR179 | CDR | PRT | U.S. Pat. No. 9,745,373; SEQ ID NO: 126 | 8353 |
| MGR180 | CDR | PRT | U.S. Pat. No. 9,745,373; SEQ ID NO: 130 | 8354 |
| MGR181 | CDR | PRT | U.S. Pat. No. 9,745,373; SEQ ID NO: 127 | 8355 |
| MGR182 | CDR | PRT | U.S. Pat. No. 9,745,373; SEQ ID NO: 107 | 8356 |
| MGR183 | CDR | PRT | U.S. Pat. No. 9,745,373; SEQ ID NO: 137 | 8357 |
| MGR184 | CDR | PRT | U.S. Pat. No. 9,745,373; SEQ ID NO: 17 | 8358 |
| MGR185 | CDR | PRT | U.S. Pat. No. 9,745,373; SEQ ID NO: 67 | 8359 |
| MGR186 | CDR | PRT | U.S. Pat. No. 9,745,373; SEQ ID NO: 68 | 8360 |
| MGR187 | CDR | PRT | U.S. Pat. No. 9,745,373; SEQ ID NO: 108 | 8361 |
| MGR188 | CDR | PRT | U.S. Pat. No. 9,745,373; SEQ ID NO: 125 | 8362 |
| MGR189 | CDR | PRT | U.S. Pat. No. 9,745,373; SEQ ID NO: 135 | 8363 |
| MGR190 | CDR | PRT | U.S. Pat. No. 9,745,373; SEQ ID NO: 15 | 8364 |
| MGR191 | CDR | PRT | U.S. Pat. No. 9,745,373; SEQ ID NO: 65 | 8365 |
| MGR192 | CDR | PRT | U.S. Pat. No. 9,745,373; SEQ ID NO: 105 | 8366 |
| MGR193 | CDR | PRT | U.S. Pat. No. 9,745,373; SEQ ID NO: 128 | 8367 |
| MGR194 | CDR | PRT | U.S. Pat. No. 9,745,373; SEQ ID NO: 106 | 8368 |
| MGR195 | CDR | PRT | U.S. Pat. No. 9,745,373; SEQ ID NO: 138 | 8369 |
| MGR196 | CDR | PRT | U.S. Pat. No. 9,745,373; SEQ ID NO: 19 | 8370 |
| MGR197 | CDR | PRT | U.S. Pat. No. 9,745,373; SEQ ID NO: 39 | 8371 |
| MGR198 | CDR | PRT | U.S. Pat. No. 9,745,373; SEQ ID NO: 139 | 8372 |
| MGR199 | CDR | PRT | U.S. Pat. No. 9,745,373; SEQ ID NO: 109 | 8373 |
| MGR200 | CDR | PRT | U.S. Pat. No. 9,745,373; SEQ ID NO: 69 | 8374 |
| MGR201 | HC | DNA | US20150322142; SEQ ID NO: 13 | 8375 |
| MGR202 | HC | DNA | U.S. Pat. No. 9,102,731; SEQ ID NO: 252 | 8376 |
| MGR203 | HC | DNA | U.S. Pat. No. 9,102,731; SEQ ID NO: 242 | 8377 |
| MGR204 | HC | DNA | U.S. Pat. No. 9,102,731; SEQ ID NO: 256 | 8378 |
| MGR205 | HC | DNA | U.S. Pat. No. 9,102,731; SEQ ID NO: 246 | 8379 |
| MGR206 | HC | DNA | U.S. Pat. No. 9,102,731; SEQ ID NO: 258 | 8380 |
| MGR207 | HC | DNA | U.S. Pat. No. 9,102,731; SEQ ID NO: 245 | 8381 |
| MGR208 | HC | DNA | U.S. Pat. No. 9,102,731; SEQ ID NO: 253 | 8382 |
| MGR209 | HC | DNA | U.S. Pat. No. 9,102,731; SEQ ID NO: 254 | 8383 |
| MGR210 | HC | DNA | U.S. Pat. No. 9,102,731; SEQ ID NO: 244 | 8384 |
| MGR211 | HC | DNA | U.S. Pat. No. 9,102,731; SEQ ID NO: 257 | 8385 |
| MGR212 | HC | DNA | U.S. Pat. No. 9,102,731; SEQ ID NO: 248 | 8386 |
| MGR213 | HC | DNA | U.S. Pat. No. 9,102,731; SEQ ID NO: 251 | 8387 |
| MGR214 | HC | DNA | U.S. Pat. No. 9,102,731; SEQ ID NO: 243 | 8388 |
| MGR215 | HC | DNA | U.S. Pat. No. 9,102,731; SEQ ID NO: 259 | 8389 |
| MGR216 | HC | DNA | U.S. Pat. No. 9,745,373; SEQ ID NO: 204 | 8390 |
| MGR217 | HC | DNA | U.S. Pat. No. 9,745,373; SEQ ID NO: 244 | 8391 |
| MGR218 | HC | DNA | U.S. Pat. No. 9,745,373; SEQ ID NO: 224 | 8392 |
| MGR219 | HC | DNA | U.S. Pat. No. 9,745,373; SEQ ID NO: 144 | 8393 |
| MGR220 | HC | DNA | U.S. Pat. No. 9,745,373; SEQ ID NO: 173 | 8394 |
| MGR221 | HC | DNA | U.S. Pat. No. 9,745,373; SEQ ID NO: 174 | 8395 |
| MGR222 | HC | DNA | U.S. Pat. No. 9,745,373; SEQ ID NO: 264 | 8396 |
| MGR223 | HC | DNA | WO2007054809; SEQ ID NO: 9 | 8397 |
| MGR224 | HC | DNA | U.S. Pat. No. 9,745,373; SEQ ID NO: 184 | 8398 |
| MGR225 | HC | DNA | U.S. Pat. No. 9,745,373; SEQ ID NO: 194 | 8399 |
| MGR226 | HC | DNA | U.S. Pat. No. 9,745,373; SEQ ID NO: 254 | 8400 |
| MGR227 | HC | DNA | U.S. Pat. No. 9,745,373; SEQ ID NO: 234 | 8401 |
| MGR228 | HC | DNA | U.S. Pat. No. 9,745,373; SEQ ID NO: 274 | 8402 |
| MGR229 | HC | DNA | U.S. Pat. No. 9,745,373; SEQ ID NO: 154 | 8403 |
| MGR230 | HC | DNA | U.S. Pat. No. 9,745,373; SEQ ID NO: 164 | 8404 |
| MGR231 | HC | PRT | U.S. Pat. No. 9,073,991; SEQ ID NO: 32 | 8405 |
| MGR232 | HC | PRT | U.S. Pat. No. 9,073,991; SEQ ID NO: 35 | 8406 |
| MGR233 | HC | PRT | U.S. Pat. No. 9,073,991; SEQ ID NO: 33 | 8407 |
| MGR234 | HC | PRT | U.S. Pat. No. 9,073,991; SEQ ID NO: 34 | 8408 |
| MGR235 | HC | PRT | U.S. Pat. No. 9,073,991; SEQ ID NO: 36 | 8409 |
| MGR236 | HC | PRT | WO2007076336; SEQ ID NO: 40 | 8410 |
| MGR237 | HC | PRT | US20150322142; SEQ ID NO: 11 | 8411 |
| MGR238 | HC | PRT | US20150322142; SEQ ID NO: 1 | 8412 |
| MGR239 | HC | PRT | US20120294802; SEQ ID NO: 54 | 8413 |
| MGR240 | HC | PRT | US20120294802; SEQ ID NO: 44 | 8414 |
| MGR241 | HC | PRT | US20120294802; SEQ ID NO: 34 | 8415 |
| MGR242 | HC | PRT | US20120294802; SEQ ID NO: 14 | 8416 |
| MGR243 | HC | PRT | US20120294802; SEQ ID NO: 3 | 8417 |
| MGR244 | HC | PRT | US20120294802; SEQ ID NO: 4 | 8418 |
| MGR245 | HC | PRT | U.S. Pat. No. 9,102,731; SEQ ID NO: 31 | 8419 |
| MGR246 | HC | PRT | U.S. Pat. No. 9,102,731; SEQ ID NO: 37 | 8420 |
| MGR247 | HC | PRT | U.S. Pat. No. 9,102,731; SEQ ID NO: 38 | 8421 |
| MGR248 | HC | PRT | U.S. Pat. No. 9,102,731; SEQ ID NO: 35 | 8422 |
| MGR249 | HC | PRT | U.S. Pat. No. 9,102,731; SEQ ID NO: 30 | 8423 |
| MGR250 | HC | PRT | U.S. Pat. No. 9,102,731; SEQ ID NO: 40 | 8424 |
| MGR251 | HC | PRT | U.S. Pat. No. 9,102,731; SEQ ID NO: 34 | 8425 |
| MGR252 | HC | PRT | U.S. Pat. No. 9,102,731; SEQ ID NO: 39 | 8426 |
| MGR253 | HC | PRT | U.S. Pat. No. 9,102,731; SEQ ID NO: 29 | 8427 |
| MGR254 | HC | PRT | U.S. Pat. No. 9,102,731; SEQ ID NO: 33 | 8428 |
| MGR255 | HC | PRT | U.S. Pat. No. 9,102,731; SEQ ID NO: 41 | 8429 |
| MGR256 | HC | PRT | U.S. Pat. No. 9,102,731; SEQ ID NO: 36 | 8430 |
| MGR257 | HC | PRT | U.S. Pat. No. 9,102,731; SEQ ID NO: 32 | 8431 |
| MGR258 | HC | PRT | U.S. Pat. No. 9,896,502; SEQ ID NO: 11 | 8432 |
| MGR259 | HC | PRT | U.S. Pat. No. 9,745,373; SEQ ID NO: 284 | 8433 |
| MGR260 | HC | PRT | U.S. Pat. No. 9,745,373; SEQ ID NO: 43 | 8434 |
| MGR261 | HC | PRT | U.S. Pat. No. 9,745,373; SEQ ID NO: 74 | 8435 |
| MGR262 | HC | PRT | U.S. Pat. No. 9,745,373; SEQ ID NO: 114 | 8436 |
| MGR263 | HC | PRT | U.S. Pat. No. 9,745,373; SEQ ID NO: 134 | 8437 |
| MGR264 | HC | PRT | U.S. Pat. No. 9,745,373; SEQ ID NO: 94 | 8438 |
| MGR265 | HC | PRT | U.S. Pat. No. 9,745,373; SEQ ID NO: 24 | 8439 |
| MGR266 | HC | PRT | U.S. Pat. No. 9,745,373; SEQ ID NO: 14 | 8440 |
| MGR267 | HC | PRT | U.S. Pat. No. 9,745,373; SEQ ID NO: 23 | 8441 |
| MGR268 | HC | PRT | U.S. Pat. No. 9,745,373; SEQ ID NO: 64 | 8442 |
| MGR269 | HC | PRT | U.S. Pat. No. 9,745,373; SEQ ID NO: 104 | 8443 |
| MGR270 | HC | PRT | U.S. Pat. No. 9,745,373; SEQ ID NO: 33 | 8444 |
| MGR271 | HC | PRT | U.S. Pat. No. 9,745,373; SEQ ID NO: 84 | 8445 |
| MGR272 | HC | PRT | U.S. Pat. No. 9,745,373; SEQ ID NO: 3 | 8446 |
| MGR273 | HC | PRT | U.S. Pat. No. 9,745,373; SEQ ID NO: 124 | 8447 |
| MGR274 | HC | PRT | WO2007076336; SEQ ID NO: 41 | 8448 |
| MGR275 | HC | PRT | U.S. Pat. No. 9,862,771; SEQ ID NO: 35 | 8449 |
| MGR276 | HC | PRT | U.S. Pat. No. 9,862,771; SEQ ID NO: 39 | 8450 |
| MGR277 | HC | PRT | U.S. Pat. No. 9,862,771; SEQ ID NO: 29 | 8451 |
| MGR278 | HC | PRT | U.S. Pat. No. 9,862,771; SEQ ID NO: 36 | 8452 |
| MGR279 | HC | PRT | U.S. Pat. No. 9,862,771; SEQ ID NO: 32 | 8453 |
| MGR280 | LC | DNA | US20150322142; SEQ ID NO: 14 | 8454 |
| MGR281 | LC | DNA | U.S. Pat. No. 9,102,731; SEQ ID NO: 241 | 8455 |
| MGR282 | LC | DNA | U.S. Pat. No. 9,102,731; SEQ ID NO: 240 | 8456 |
| MGR283 | LC | DNA | U.S. Pat. No. 9,102,731; SEQ ID NO: 235 | 8457 |
| MGR284 | LC | DNA | U.S. Pat. No. 9,102,731; SEQ ID NO: 225 | 8458 |
| MGR285 | LC | DNA | U.S. Pat. No. 9,102,731; SEQ ID NO: 234 | 8459 |
| MGR286 | LC | DNA | U.S. Pat. No. 9,102,731; SEQ ID NO: 233 | 8460 |
| MGR287 | LC | DNA | U.S. Pat. No. 9,102,731; SEQ ID NO: 224 | 8461 |
| MGR288 | LC | DNA | U.S. Pat. No. 9,102,731; SEQ ID NO: 229 | 8462 |
| MGR289 | LC | DNA | U.S. Pat. No. 9,102,731; SEQ ID NO: 238 | 8463 |
| MGR290 | LC | DNA | U.S. Pat. No. 9,102,731; SEQ ID NO: 232 | 8464 |
| MGR291 | LC | DNA | U.S. Pat. No. 9,102,731; SEQ ID NO: 239 | 8465 |
| MGR292 | LC | DNA | U.S. Pat. No. 9,102,731; SEQ ID NO: 226 | 8466 |
| MGR293 | LC | DNA | U.S. Pat. No. 9,102,731; SEQ ID NO: 237 | 8467 |
| MGR294 | LC | DNA | U.S. Pat. No. 9,102,731; SEQ ID NO: 231 | 8468 |
| MGR295 | LC | DNA | U.S. Pat. No. 9,102,731; SEQ ID NO: 230 | 8469 |
| MGR296 | LC | DNA | U.S. Pat. No. 9,102,731; SEQ ID NO: 227 | 8470 |
| MGR297 | LC | DNA | U.S. Pat. No. 9,102,731; SEQ ID NO: 260 | 8471 |
| MGR298 | LC | DNA | U.S. Pat. No. 9,102,731; SEQ ID NO: 261 | 8472 |
| MGR299 | LC | DNA | U.S. Pat. No. 9,745,373; SEQ ID NO: 222 | 8473 |
| MGR300 | LC | DNA | U.S. Pat. No. 9,745,373; SEQ ID NO: 171 | 8474 |
| MGR301 | LC | DNA | U.S. Pat. No. 9,745,373; SEQ ID NO: 172 | 8475 |
| MGR302 | LC | DNA | U.S. Pat. No. 9,745,373; SEQ ID NO: 201 | 8476 |
| MGR303 | LC | DNA | U.S. Pat. No. 9,745,373; SEQ ID NO: 202 | 8477 |
| MGR304 | LC | DNA | U.S. Pat. No. 9,745,373; SEQ ID NO: 141 | 8478 |
| MGR305 | LC | DNA | U.S. Pat. No. 9,745,373; SEQ ID NO: 142 | 8479 |
| MGR306 | LC | DNA | U.S. Pat. No. 9,745,373; SEQ ID NO: 232 | 8480 |
| MGR307 | LC | DNA | U.S. Pat. No. 9,745,373; SEQ ID NO: 211 | 8481 |
| MGR308 | LC | DNA | U.S. Pat. No. 9,745,373; SEQ ID NO: 212 | 8482 |
| MGR309 | LC | DNA | U.S. Pat. No. 9,745,373; SEQ ID NO: 181 | 8483 |
| MGR310 | LC | DNA | U.S. Pat. No. 9,745,373; SEQ ID NO: 182 | 8484 |
| MGR311 | LC | DNA | U.S. Pat. No. 9,745,373; SEQ ID NO: 151 | 8485 |
| MGR312 | LC | DNA | U.S. Pat. No. 9,745,373; SEQ ID NO: 152 | 8486 |
| MGR313 | LC | DNA | U.S. Pat. No. 9,745,373; SEQ ID NO: 242 | 8487 |
| MGR314 | LC | DNA | WO2007076336; SEQ ID NO: 42 | 8488 |
| MGR315 | LC | DNA | WO2007054809; SEQ ID NO: 10 | 8489 |
| MGR316 | LC | DNA | U.S. Pat. No. 9,745,373; SEQ ID NO: 262 | 8490 |
| MGR317 | LC | PRT | U.S. Pat. No. 9,073,991; SEQ ID NO: 29 | 8491 |
| MGR318 | LC | PRT | U.S. Pat. No. 9,073,991; SEQ ID NO: 31 | 8492 |
| MGR319 | LC | PRT | U.S. Pat. No. 9,073,991; SEQ ID NO: 27 | 8493 |
| MGR320 | LC | PRT | U.S. Pat. No. 9,073,991; SEQ ID NO: 30 | 8494 |
| MGR321 | LC | PRT | US20150322142; SEQ ID NO: 12 | 8495 |
| MGR322 | LC | PRT | US20150322142; SEQ ID NO: 2 | 8496 |
| MGR323 | LC | PRT | US20120294802; SEQ ID NO: 42 | 8497 |
| MGR324 | LC | PRT | US20120294802; SEQ ID NO: 32 | 8498 |
| MGR325 | LC | PRT | US20120294802; SEQ ID NO: 12 | 8499 |
| MGR326 | LC | PRT | US20120294802; SEQ ID NO: 1 | 8500 |
| MGR327 | LC | PRT | US20120294802; SEQ ID NO: 2 | 8501 |
| MGR328 | LC | PRT | U.S. Pat. No. 9,102,731; SEQ ID NO: 16 | 8502 |
| MGR329 | LC | PRT | U.S. Pat. No. 9,102,731; SEQ ID NO: 19 | 8503 |
| MGR330 | LC | PRT | U.S. Pat. No. 9,102,731; SEQ ID NO: 26 | 8504 |
| MGR331 | LC | PRT | U.S. Pat. No. 9,102,731; SEQ ID NO: 14 | 8505 |
| MGR332 | LC | PRT | U.S. Pat. No. 9,102,731; SEQ ID NO: 24 | 8506 |
| MGR333 | LC | PRT | U.S. Pat. No. 9,102,731; SEQ ID NO: 18 | 8507 |
| MGR334 | LC | PRT | U.S. Pat. No. 9,102,731; SEQ ID NO: 22 | 8508 |
| MGR335 | LC | PRT | U.S. Pat. No. 9,102,731; SEQ ID NO: 23 | 8509 |
| MGR336 | LC | PRT | U.S. Pat. No. 9,102,731; SEQ ID NO: 13 | 8510 |
| MGR337 | LC | PRT | U.S. Pat. No. 9,102,731; SEQ ID NO: 20 | 8511 |
| MGR338 | LC | PRT | U.S. Pat. No. 9,102,731; SEQ ID NO: 25 | 8512 |
| MGR339 | LC | PRT | U.S. Pat. No. 9,102,731; SEQ ID NO: 17 | 8513 |
| MGR340 | LC | PRT | U.S. Pat. No. 9,102,731; SEQ ID NO: 12 | 8514 |
| MGR341 | LC | PRT | U.S. Pat. No. 9,102,731; SEQ ID NO: 21 | 8515 |
| MGR342 | LC | PRT | U.S. Pat. No. 9,102,731; SEQ ID NO: 15 | 8516 |
| MGR343 | LC | PRT | U.S. Pat. No. 9,102,731; SEQ ID NO: 27 | 8517 |
| MGR344 | LC | PRT | U.S. Pat. No. 9,102,731; SEQ ID NO: 28 | 8518 |
| MGR345 | LC | PRT | U.S. Pat. No. 9,896,502; SEQ ID NO: 12 | 8519 |
| MGR346 | LC | PRT | U.S. Pat. No. 9,745,373; SEQ ID NO: 122 | 8520 |
| MGR347 | LC | PRT | WO2007054809; SEQ ID NO: 12 | 8521 |
| MGR348 | LC | PRT | U.S. Pat. No. 9,745,373; SEQ ID NO: 132 | 8522 |
| MGR349 | LC | PRT | U.S. Pat. No. 9,745,373; SEQ ID NO: 91 | 8523 |
| MGR350 | LC | PRT | U.S. Pat. No. 9,745,373; SEQ ID NO: 12 | 8524 |
| MGR351 | LC | PRT | U.S. Pat. No. 9,745,373; SEQ ID NO: 21 | 8525 |
| MGR352 | LC | PRT | U.S. Pat. No. 9,745,373; SEQ ID NO: 42 | 8526 |
| MGR353 | LC | PRT | U.S. Pat. No. 9,745,373; SEQ ID NO: 51 | 8527 |
| MGR354 | LC | PRT | U.S. Pat. No. 9,745,373; SEQ ID NO: 72 | 8528 |
| MGR355 | LC | PRT | U.S. Pat. No. 9,745,373; SEQ ID NO: 71 | 8529 |
| MGR356 | LC | PRT | U.S. Pat. No. 9,745,373; SEQ ID NO: 112 | 8530 |
| MGR357 | LC | PRT | U.S. Pat. No. 9,745,373; SEQ ID NO: 2 | 8531 |
| MGR358 | LC | PRT | U.S. Pat. No. 9,745,373; SEQ ID NO: 1 | 8532 |
| MGR359 | LC | PRT | U.S. Pat. No. 9,745,373; SEQ ID NO: 62 | 8533 |
| MGR360 | LC | PRT | U.S. Pat. No. 9,745,373; SEQ ID NO: 61 | 8534 |
| MGR361 | LC | PRT | U.S. Pat. No. 9,745,373; SEQ ID NO: 102 | 8535 |
| MGR362 | LC | PRT | U.S. Pat. No. 9,745,373; SEQ ID NO: 82 | 8536 |
| MGR363 | LC | PRT | U.S. Pat. No. 9,745,373; SEQ ID NO: 32 | 8537 |
| MGR364 | LC | PRT | U.S. Pat. No. 9,745,373; SEQ ID NO: 31 | 8538 |
| MGR365 | LC | PRT | U.S. Pat. No. 9,745,373; SEQ ID NO: 283 | 8539 |
| MGR366 | LC | PRT | U.S. Pat. No. 9,862,771; SEQ ID NO: 22 | 8540 |
| MGR367 | LC | PRT | U.S. Pat. No. 9,862,771; SEQ ID NO: 25 | 8541 |
| MGR368 | LC | PRT | U.S. Pat. No. 9,862,771; SEQ ID NO: 17 | 8542 |
| MGR369 | LC | PRT | U.S. Pat. No. 9,862,771; SEQ ID NO: 21 | 8543 |
| MGR370 | LC | PRT | U.S. Pat. No. 9,862,771; SEQ ID NO: 15 | 8544 |
| MGR371 | VH | DNA | U.S. Pat. No. 9,102,731; SEQ ID NO: 228 | 8545 |
| MGR372 | VH | DNA | U.S. Pat. No. 9,102,731; SEQ ID NO: 212 | 8546 |
| MGR373 | VH | DNA | U.S. Pat. No. 9,102,731; SEQ ID NO: 197 | 8547 |
| MGR374 | VH | DNA | U.S. Pat. No. 9,102,731; SEQ ID NO: 198 | 8548 |
| MGR375 | VH | DNA | U.S. Pat. No. 9,102,731; SEQ ID NO: 207 | 8549 |
| MGR376 | VH | DNA | U.S. Pat. No. 9,102,731; SEQ ID NO: 208 | 8550 |
| MGR377 | VH | DNA | U.S. Pat. No. 9,102,731; SEQ ID NO: 206 | 8551 |
| MGR378 | VH | DNA | U.S. Pat. No. 9,102,731; SEQ ID NO: 210 | 8552 |
| MGR379 | VH | DNA | U.S. Pat. No. 9,102,731; SEQ ID NO: 211 | 8553 |
| MGR380 | VH | DNA | U.S. Pat. No. 9,102,731; SEQ ID NO: 196 | 8554 |
| MGR381 | VH | DNA | U.S. Pat. No. 9,102,731; SEQ ID NO: 199 | 8555 |
| MGR382 | VH | DNA | U.S. Pat. No. 9,102,731; SEQ ID NO: 200 | 8556 |
| MGR383 | VH | DNA | U.S. Pat. No. 9,102,731; SEQ ID NO: 202 | 8557 |
| MGR384 | VH | DNA | U.S. Pat. No. 9,102,731; SEQ ID NO: 205 | 8558 |
| MGR385 | VH | DNA | U.S. Pat. No. 9,102,731; SEQ ID NO: 204 | 8559 |
| MGR386 | VH | DNA | U.S. Pat. No. 9,102,731; SEQ ID NO: 209 | 8560 |
| MGR387 | VH | DNA | U.S. Pat. No. 9,102,731; SEQ ID NO: 201 | 8561 |
| MGR388 | VH | DNA | U.S. Pat. No. 9,102,731; SEQ ID NO: 203 | 8562 |
| MGR389 | VH | DNA | U.S. Pat. No. 9,745,373; SEQ ID NO: 203 | 8563 |
| MGR390 | VH | DNA | U.S. Pat. No. 9,745,373; SEQ ID NO: 243 | 8564 |
| MGR391 | VH | DNA | U.S. Pat. No. 9,745,373; SEQ ID NO: 223 | 8565 |
| MGR392 | VH | DNA | U.S. Pat. No. 9,745,373; SEQ ID NO: 263 | 8566 |
| MGR393 | VH | DNA | U.S. Pat. No. 9,745,373; SEQ ID NO: 183 | 8567 |
| MGR394 | VH | DNA | U.S. Pat. No. 9,745,373; SEQ ID NO: 253 | 8568 |
| MGR395 | VH | DNA | U.S. Pat. No. 9,745,373; SEQ ID NO: 153 | 8569 |
| MGR396 | VH | PRT | WO2007076336; SEQ ID NO: 39 | 8570 |
| MGR397 | VH | PRT | US20150322142; SEQ ID NO: 79 | 8571 |
| MGR398 | VH | PRT | US20150322142; SEQ ID NO: 59 | 8572 |
| MGR399 | VH | PRT | US20150322142; SEQ ID NO: 65 | 8573 |
| MGR400 | VH | PRT | US20150322142; SEQ ID NO: 61 | 8574 |
| MGR401 | VH | PRT | US20150322142; SEQ ID NO: 63 | 8575 |
| MGR402 | VH | PRT | US20150322142; SEQ ID NO: 67 | 8576 |
| MGR403 | VH | PRT | WO2007076336; SEQ ID NO: 35 | 8577 |
| MGR404 | VH | PRT | WO2007076336; SEQ ID NO: 37 | 8578 |
| MGR405 | VH | PRT | WO2007076336; SEQ ID NO: 36 | 8579 |
| MGR406 | VH | PRT | WO2007076336; SEQ ID NO: 38 | 8580 |
| MGR407 | VH | PRT | WO2007076336; SEQ ID NO: 34 | 8581 |
| MGR408 | VH | PRT | US20120294802; SEQ ID NO: 43 | 8582 |
| MGR409 | VH | PRT | US20120294802; SEQ ID NO: 33 | 8583 |
| MGR410 | VH | PRT | US20120294802; SEQ ID NO: 13 | 8584 |
| MGR411 | VH | PRT | U.S. Pat. No. 9,102,731; SEQ ID NO: 160 | 8585 |
| MGR412 | VH | PRT | U.S. Pat. No. 9,102,731; SEQ ID NO: 166 | 8586 |
| MGR413 | VH | PRT | U.S. Pat. No. 9,102,731; SEQ ID NO: 167 | 8587 |
| MGR414 | VH | PRT | U.S. Pat. No. 9,102,731; SEQ ID NO: 164 | 8588 |
| MGR415 | VH | PRT | U.S. Pat. No. 9,102,731; SEQ ID NO: 159 | 8589 |
| MGR416 | VH | PRT | U.S. Pat. No. 9,102,731; SEQ ID NO: 169 | 8590 |
| MGR417 | VH | PRT | U.S. Pat. No. 9,102,731; SEQ ID NO: 163 | 8591 |
| MGR418 | VH | PRT | U.S. Pat. No. 9,102,731; SEQ ID NO: 174 | 8592 |
| MGR419 | VH | PRT | U.S. Pat. No. 9,102,731; SEQ ID NO: 172 | 8593 |
| MGR420 | VH | PRT | U.S. Pat. No. 9,102,731; SEQ ID NO: 168 | 8594 |
| MGR421 | VH | PRT | U.S. Pat. No. 9,102,731; SEQ ID NO: 158 | 8595 |
| MGR422 | VH | PRT | U.S. Pat. No. 9,102,731; SEQ ID NO: 162 | 8596 |
| MGR423 | VH | PRT | U.S. Pat. No. 9,102,731; SEQ ID NO: 170 | 8597 |
| MGR424 | VH | PRT | U.S. Pat. No. 9,102,731; SEQ ID NO: 171 | 8598 |
| MGR425 | VH | PRT | U.S. Pat. No. 9,102,731; SEQ ID NO: 165 | 8599 |
| MGR426 | VH | PRT | U.S. Pat. No. 9,102,731; SEQ ID NO: 161 | 8600 |
| MGR427 | VH | PRT | U.S. Pat. No. 9,102,731; SEQ ID NO: 173 | 8601 |
| MGR428 | VH | PRT | U.S. Pat. No. 9,896,502; SEQ ID NO: 1 | 8602 |
| MGR429 | VH | PRT | U.S. Pat. No. 9,896,502; SEQ ID NO: 71 | 8603 |
| MGR430 | VH | PRT | U.S. Pat. No. 9,896,502; SEQ ID NO: 59 | 8604 |
| MGR431 | VH | PRT | U.S. Pat. No. 9,896,502; SEQ ID NO: 65 | 8605 |
| MGR432 | VH | PRT | U.S. Pat. No. 9,896,502; SEQ ID NO: 61 | 8606 |
| MGR433 | VH | PRT | U.S. Pat. No. 9,896,502; SEQ ID NO: 63 | 8607 |
| MGR434 | VH | PRT | U.S. Pat. No. 9,896,502; SEQ ID NO: 67 | 8608 |
| MGR435 | VH | PRT | U.S. Pat. No. 9,745,373; SEQ ID NO: 73 | 8609 |
| MGR436 | VH | PRT | U.S. Pat. No. 9,745,373; SEQ ID NO: 113 | 8610 |
| MGR437 | VH | PRT | U.S. Pat. No. 9,745,373; SEQ ID NO: 133 | 8611 |
| MGR438 | VH | PRT | U.S. Pat. No. 9,745,373; SEQ ID NO: 63 | 8612 |
| MGR439 | VH | PRT | U.S. Pat. No. 9,745,373; SEQ ID NO: 103 | 8613 |
| MGR440 | VH | PRT | U.S. Pat. No. 9,745,373; SEQ ID NO: 83 | 8614 |
| MGR441 | VH | PRT | U.S. Pat. No. 9,745,373; SEQ ID NO: 123 | 8615 |
| MGR442 | VH | PRT | U.S. Pat. No. 9,862,771; SEQ ID NO: 164 | 8616 |
| MGR443 | VH | PRT | U.S. Pat. No. 9,862,771; SEQ ID NO: 168 | 8617 |
| MGR444 | VH | PRT | U.S. Pat. No. 9,862,771; SEQ ID NO: 165 | 8618 |
| MGR445 | VH | PRT | U.S. Pat. No. 9,862,771; SEQ ID NO: 161 | 8619 |
| MGR446 | VI | DNA | US20150322142; SEQ ID NO: 10 | 8620 |
| MGR447 | VL | DNA | U.S. Pat. No. 9,102,731; SEQ ID NO: 177 | 8621 |
| MGR448 | VL | DNA | U.S. Pat. No. 9,102,731; SEQ ID NO: 192 | 8622 |
| MGR449 | VL | DNA | U.S. Pat. No. 9,102,731; SEQ ID NO: 193 | 8623 |
| MGR450 | VL | DNA | U.S. Pat. No. 9,102,731; SEQ ID NO: 191 | 8624 |
| MGR451 | VL | DNA | U.S. Pat. No. 9,102,731; SEQ ID NO: 194 | 8625 |
| MGR452 | VL | DNA | U.S. Pat. No. 9,102,731; SEQ ID NO: 190 | 8626 |
| MGR453 | VL | DNA | U.S. Pat. No. 9,102,731; SEQ ID NO: 186 | 8627 |
| MGR454 | VL | DNA | U.S. Pat. No. 9,102,731; SEQ ID NO: 187 | 8628 |
| MGR455 | VL | DNA | U.S. Pat. No. 9,102,731; SEQ ID NO: 189 | 8629 |
| MGR456 | VL | DNA | U.S. Pat. No. 9,102,731; SEQ ID NO: 188 | 8630 |
| MGR457 | VL | DNA | U.S. Pat. No. 9,102,731; SEQ ID NO: 184 | 8631 |
| MGR458 | VL | DNA | U.S. Pat. No. 9,102,731; SEQ ID NO: 183 | 8632 |
| MGR459 | VL | DNA | U.S. Pat. No. 9,102,731; SEQ ID NO: 182 | 8633 |
| MGR460 | VL | DNA | U.S. Pat. No. 9,102,731; SEQ ID NO: 176 | 8634 |
| MGR461 | VL | DNA | U.S. Pat. No. 9,102,731; SEQ ID NO: 175 | 8635 |
| MGR462 | VL | DNA | U.S. Pat. No. 9,102,731; SEQ ID NO: 185 | 8636 |
| MGR463 | VL | DNA | U.S. Pat. No. 9,102,731; SEQ ID NO: 181 | 8637 |
| MGR464 | VL | DNA | U.S. Pat. No. 9,102,731; SEQ ID NO: 179 | 8638 |
| MGR465 | VL | DNA | U.S. Pat. No. 9,102,731; SEQ ID NO: 180 | 8639 |
| MGR466 | VL | DNA | U.S. Pat. No. 9,102,731; SEQ ID NO: 178 | 8640 |
| MGR467 | VL | DNA | U.S. Pat. No. 9,102,731; SEQ ID NO: 195 | 8641 |
| MGR468 | VL | DNA | U.S. Pat. No. 9,745,373; SEQ ID NO: 221 | 8642 |
| MGR469 | VL | DNA | U.S. Pat. No. 9,745,373; SEQ ID NO: 231 | 8643 |
| MGR470 | VL | DNA | U.S. Pat. No. 9,745,373; SEQ ID NO: 241 | 8644 |
| MGR471 | VL | DNA | U.S. Pat. No. 9,745,373; SEQ ID NO: 261 | 8645 |
| MGR472 | VL | PRT | US20150322142; SEQ ID NO: 62 | 8646 |
| MGR473 | VL | PRT | US20150322142; SEQ ID NO: 66 | 8647 |
| MGR474 | VL | PRT | US20150322142; SEQ ID NO: 58 | 8648 |
| MGR475 | VL | PRT | US20150322142; SEQ ID NO: 64 | 8649 |
| MGR476 | VL | PRT | WO2007076336; SEQ ID NO: 29 | 8650 |
| MGR477 | VL | PRT | WO2007076336; SEQ ID NO: 28 | 8651 |
| MGR478 | VL | PRT | WO2007076336; SEQ ID NO: 33 | 8652 |
| MGR479 | VL | PRT | US20150322142; SEQ ID NO: 78 | 8653 |
| MGR480 | VL | PRT | US20120294802; SEQ ID NO: 41 | 8654 |
| MGR481 | VL | PRT | US20120294802; SEQ ID NO: 31 | 8655 |
| MGR482 | VL | PRT | US20120294802; SEQ ID NO: 11 | 8656 |
| MGR483 | VL | PRT | U.S. Pat. No. 9,102,731; SEQ ID NO: 141 | 8657 |
| MGR484 | VL | PRT | U.S. Pat. No. 9,102,731; SEQ ID NO: 144 | 8658 |
| MGR485 | VL | PRT | U.S. Pat. No. 9,102,731; SEQ ID NO: 151 | 8659 |
| MGR486 | VL | PRT | U.S. Pat. No. 9,102,731; SEQ ID NO: 139 | 8660 |
| MGR487 | VL | PRT | U.S. Pat. No. 9,102,731; SEQ ID NO: 155 | 8661 |
| MGR488 | VL | PRT | U.S. Pat. No. 9,102,731; SEQ ID NO: 149 | 8662 |
| MGR489 | VL | PRT | U.S. Pat. No. 9,102,731; SEQ ID NO: 154 | 8663 |
| MGR490 | VL | PRT | U.S. Pat. No. 9,102,731; SEQ ID NO: 143 | 8664 |
| MGR491 | VL | PRT | U.S. Pat. No. 9,102,731; SEQ ID NO: 152 | 8665 |
| MGR492 | VL | PRT | U.S. Pat. No. 9,102,731; SEQ ID NO: 153 | 8666 |
| MGR493 | VL | PRT | U.S. Pat. No. 9,102,731; SEQ ID NO: 156 | 8667 |
| MGR494 | VL | PRT | U.S. Pat. No. 9,102,731; SEQ ID NO: 147 | 8668 |
| MGR495 | VL | PRT | U.S. Pat. No. 9,102,731; SEQ ID NO: 148 | 8669 |
| MGR496 | VL | PRT | U.S. Pat. No. 9,102,731; SEQ ID NO: 138 | 8670 |
| MGR497 | VL | PRT | U.S. Pat. No. 9,102,731; SEQ ID NO: 157 | 8671 |
| MGR498 | VL | PRT | U.S. Pat. No. 9,102,731; SEQ ID NO: 145 | 8672 |
| MGR499 | VL | PRT | U.S. Pat. No. 9,102,731; SEQ ID NO: 150 | 8673 |
| MGR500 | VL | PRT | U.S. Pat. No. 9,102,731; SEQ ID NO: 137 | 8674 |
| MGR501 | VL | PRT | U.S. Pat. No. 9,102,731; SEQ ID NO: 146 | 8675 |
| MGR502 | VL | PRT | U.S. Pat. No. 9,102,731; SEQ ID NO: 140 | 8676 |
| MGR503 | VL | PRT | U.S. Pat. No. 9,896,502; SEQ ID NO: 2 | 8677 |
| MGR504 | VL | PRT | U.S. Pat. No. 9,745,373; SEQ ID NO: 121 | 8678 |
| MGR505 | VL | PRT | U.S. Pat. No. 9,745,373; SEQ ID NO: 111 | 8679 |
| MGR506 | VL | PRT | U.S. Pat. No. 9,745,373; SEQ ID NO: 101 | 8680 |
| MGR507 | VL | PRT | U.S. Pat. No. 9,745,373; SEQ ID NO: 81 | 8681 |
| MGR508 | VL | PRT | U.S. Pat. No. 9,862,771; SEQ ID NO: 147 | 8682 |
| MGR509 | VL | PRT | U.S. Pat. No. 9,862,771; SEQ ID NO: 150 | 8683 |
| MGR510 | VL | PRT | U.S. Pat. No. 9,862,771; SEQ ID NO: 142 | 8684 |
| MGR511 | VL | PRT | U.S. Pat. No. 9,862,771; SEQ ID NO: 146 | 8685 |
| MGR512 | VL | PRT | U.S. Pat. No. 9,862,771; SEQ ID NO: 140 | 8686 |
| MGR513 | VR | PRT | U.S. Pat. No. 9,896,502; SEQ ID NO: 62 | 8687 |
| MGR514 | VR | PRT | U.S. Pat. No. 9,896,502; SEQ ID NO: 66 | 8688 |
| MGR515 | VR | PRT | U.S. Pat. No. 9,896,502; SEQ ID NO: 60 | 8689 |
| MGR516 | VR | PRT | U.S. Pat. No. 9,896,502; SEQ ID NO: 64 | 8690 |
| MGR517 | HC | PRT | U.S. Pat. No. 9,102,731; SEQ ID NO: 32 | 8691 |
| MGR518 | HC | PRT | U.S. Pat. No. 9,102,731; SEQ ID NO: 34 | 8692 |
| MGR519 | HC | PRT | U.S. Pat. No. 9,102,731; SEQ ID NO: 37 | 8693 |
| MGR520 | HC | PRT | US20150322142; SEQ ID NO: 65 | 8694 |
| MGR521 | LC | PRT | U.S. Pat. No. 9,102,731; SEQ ID NO: 18 | 8695 |
| MGR522 | LC | PRT | U.S. Pat. No. 9,102,731; SEQ ID NO: 23 | 8696 |
| MGR523 | VH | PRT | U.S. Pat. No. 9,102,731; SEQ ID NO: 159 | 8697 |
| MGR524 | VH | PRT | U.S. Pat. No. 9,102,731; SEQ ID NO: 160 | 8698 |
| MGR525 | VH | PRT | U.S. Pat. No. 9,102,731; SEQ ID NO: 162 | 8699 |
| MGR526 | VH | PRT | U.S. Pat. No. 9,102,731; SEQ ID NO: 163 | 8700 |
| MGR527 | VH | PRT | U.S. Pat. No. 9,102,731; SEQ ID NO: 166 | 8701 |
| MGR528 | VH | PRT | U.S. Pat. No. 9,102,731; SEQ ID NO: 167 | 8702 |
| MGR529 | VH | PRT | U.S. Pat. No. 9,102,731; SEQ ID NO: 169 | 8703 |
| MGR530 | VH | PRT | U.S. Pat. No. 9,102,731; SEQ ID NO: 170 | 8704 |
| MGR531 | VL | PRT | U.S. Pat. No. 9,102,731; SEQ ID NO: 137 | 8705 |
| MGR532 | VL | PRT | U.S. Pat. No. 9,102,731; SEQ ID NO: 138 | 8706 |
| MGR533 | VL | PRT | U.S. Pat. No. 9,102,731; SEQ ID NO: 139 | 8707 |
| MGR534 | VL | PRT | U.S. Pat. No. 9,102,731; SEQ ID NO: 141 | 8708 |
| MGR535 | VL | PRT | U.S. Pat. No. 9,102,731; SEQ ID NO: 143 | 8709 |
| MGR536 | VL | PRT | U.S. Pat. No. 9,102,731; SEQ ID NO: 144 | 8710 |
| MGR537 | VL | PRT | U.S. Pat. No. 9,102,731; SEQ ID NO: 145 | 8711 |
| MGR538 | VL | PRT | U.S. Pat. No. 9,102,731; SEQ ID NO: 148 | 8712 |
| MGR539 | VL | PRT | U.S. Pat. No. 9,102,731; SEQ ID NO: 149 | 8713 |
| MGR540 | VL | PRT | U.S. Pat. No. 9,102,731; SEQ ID NO: 151 | 8714 |
| MGR541 | VL | PRT | U.S. Pat. No. 9,102,731; SEQ ID NO: 152 | 8715 |
| MGR542 | VL | PRT | U.S. Pat. No. 9,102,731; SEQ ID NO: 153 | 8716 |

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 12, or variants or fragments thereof. As another non-limiting example, the antibody may be one or more of the heavy chain sequences listed in Table 12. As a non-limiting example, the antibody may be one or more of the light chain sequences listed in Table 12, or variants or fragments thereof.

In some embodiments, the payload region of the AAV particle comprises a nucleic acid sequence encoding a polypeptide comprising a heavy chain and a light chain sequence listed in Table 12, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Table 12, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5 to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 12, one or more linkers from Table 2 and a heavy chain sequence from Table 12.

In some embodiments, the payload region comprises, in the 5 to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5 to 3′ direction, an antibody heavy chain sequence from Table 12, one or more linkers from Table 2, and alight chain sequence from Table 12.

In some embodiments, the payload region comprises a nucleic acid sequence encoding a single heavy chain. As a non-limiting example, the heavy chain is an amino acid sequence or fragment thereof described in Table 12.

Shown in Table 12 are a listing of antibodies and their polynucleotides and/or polypeptides sequences. These sequences may be encoded by or included in the AAV particles of the present disclosure. Variants or fragments of the antibody sequences described in Table 12 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 12. CDRs of the antibodies encoded by the viral genomes of the present disclosure may be 50%, 60%, 70%, 80%, 90%, 95% identical to CDRs listed in or incorporated in the sequences of Table 12. Methods of determining CDRs are well known in the art and are described herein. Payload regions may encode antibody variants with one or more heavy chain variable domain (V_{H}) or light chain variable domain (V_{L}) derived from the antibody sequences in Table 12. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any migraine-associated antibodies, not limited to those described in Table 12, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the migraine-associated antibodies as described in International Publication Number WO2017075119, WO2017085035, WO2017106578, WO2017181031, WO2017181039, WO2017189959, WO2018039506, WO2018119246, WO2018160896, WO2018166495, WO2018167322, WO2019057992, WO2019067293, WO2015015003, WO2016125017, and WO2018206790, the contents of each of which are herein incorporated by reference in their entirety.

In some embodiments, the AAV particles may have a payload region comprising any of the migraine-associated antibodies as described in US Patent Number U.S. Ser. No. 10/000,572, the contents of which are herein incorporated by reference in their entirety.

In some embodiments, the AAV particles may have a payload region comprising any of the migraine-associated antibodies as described in US Patent Publication Number US20180327487, US20160251438, and US20160272717, the contents of each of which are herein incorporated by reference in their entirety.

In some embodiments, payloads may encode migraine-associated antibodies (or fragments thereof) taught in U.S. Pat. No. 9,896,502, the contents of which are herein incorporated by reference in their entirety. Such embodiments may include antibody TEV-48125 or Fremanezumab, or fragments thereof. In certain embodiments, the payload region encodes antibody TEV-48125 or Fremanezumab, or fragments thereof selected from SEQ ID NO: 1-79, as described in U59896502.

In some embodiments, payloads may encode migraine-associated antibodies (or fragments thereof) taught in U.S. Pat. No. 9,745,373, the contents of each of which are herein incorporated by reference in their entirety. Such embodiments may include antibody ALD403 or Eptinezumab, or fragments thereof. In certain embodiments, the payload region encodes antibody ALD403 or Eptinezumab, or fragments thereof selected from SEQ ID NO: 1-284 as described in U.S. Pat. No. 9,745,373.

In some embodiments, payloads may encode migraine-associated antibodies (or fragments thereof) taught in U.S. Pat. No. 9,102,731, the contents of each of which are herein incorporated by reference in their entirety. Such embodiments may include antibody Erenumab or Aimovig or AMG-334, or fragments thereof. In certain embodiments, the payload region encodes antibody Erenumab or Aimovig or AMG-334, or fragments thereof selected from SEQ ID NO: 12-261 as described in U.S. Pat. No. 9,102,731.

In some embodiments, payloads may encode migraine-associated antibodies (or fragments thereof) taught in International Publication Number WO2007076336, the contents of which are herein incorporated by reference in their entirety. Such embodiments may include antibody Galcanezumab, or fragments thereof. In certain embodiments, the payload region encodes antibody Galcanezumab, or fragments thereof selected from SEQ ID NO: 8-42 as described in WO2007076336.

In some embodiments, payloads may encode migraine-associated antibodies (or fragments thereof) taught in International Publication Number WO2015015003, WO2016125017, and WO20120670, the contents of each of which are herein incorporated by reference in their entirety. Such embodiments may include antibody ARGX-15, Fremanezumab or fragments thereof.

In some embodiments, payloads may encode migraine-associated antibodies (or fragments thereof) taught in US Patent Publication Number US20180327487, U.S. Ser. No. 10/000,572, US20160251438, and US20160272717, the contents of each of which are herein incorporated by reference in their entirety. Such embodiments may include antibody ARGX-115, or fragments thereof.

In some embodiments, the payload region of the AAV particle comprises one or more nucleic acid sequences encoding pain-associated antibodies, variants or fragments thereof.

In some embodiments, the payload region of the AAV particle comprises one or more nucleic acid sequences encoding one or more of the payload antibody polypeptides listed in Table 13, or variants or fragments thereof. As used herein, “antibody polynucleotide” refers to a nucleic acid sequence encoding an antibody polypeptide.

In some embodiments, the payload region of the AAV particle comprises one or more nucleic acid sequences listed in Table 13, or variants or fragments thereof.

In some embodiments, the payload region of the AAV particle comprises a nucleic acid sequence encoding a payload antibody with at least 50% identity to one or more payload antibody polypeptides listed in Table 13. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 13, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 13, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%,83%, 84%,85%, 86%,87%, 88%, 89%, 90%, 91%, 92%, 93%, 94%, 95%, 96%, 97%, 98%, 99%, or 100% identity to one or more of the payload antibody polypeptides listed in Table 13, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%,99%, or 100% identity to one or more of the payload heavy chain antibody polypeptides listed in Table 13, or variants or fragments thereof.

In some embodiments, the light chain of the encoded antibody polypeptide may have 50%, 51%, 52%,53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%, 78%, 79%, 80%, 81%, 82%, 83%, 84%, 85%%,6, 87%,88%, 89%, 90%,91%, 92%, 93%,94%,95%, 96%,97%,98%, 99%, or 100% identity to one or more of the payload light chain antibody polypeptides listed in Table 13, or variants or fragments thereof.

In some embodiments, the CDR region of the encoded antibody polypeptide may have 50%, 51%, 52%, 53%, 54%, 55%, 56%, 57%, 58%, 59%, 60%, 61%,62%, 63%,64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 13, or variants or fragments thereof.

In some embodiments, the payload antibody has 90% identity to one or more of the antibody polypeptides listed in Table 13, or variants or fragments thereof.

In some embodiments, the payload antibody has 91% identity to one or more of the antibody polypeptides listed in Table 13, or variants or fragments thereof.

In some embodiments, the payload antibody has 92% identity to one or more of the antibody polypeptides listed in Table 13, or variants or fragments thereof.

In some embodiments, the payload antibody has 93% identity to one or more of the antibody polypeptides listed in Table 13, or variants or fragments thereof.

In some embodiments, the payload antibody has 94% identity to one or more of the antibody polypeptides listed in Table 13, or variants or fragments thereof.

In some embodiments, the payload antibody has 95% identity to one or more of the antibody polypeptides listed in Table 13, or variants or fragments thereof.

In some embodiments, the payload antibody has 96% identity to one or more of the antibody polypeptides listed in Table 13, or variants or fragments thereof.

In some embodiments, the payload antibody has 97% identity to one or more of the antibody polypeptides listed in Table 13, or variants or fragments thereof.

In some embodiments, the payload antibody has 98% identity to one or more of the antibody polypeptides listed in Table 13, or variants or fragments thereof.

In some embodiments, the payload antibody has 99% identity to one or more of the antibody polypeptides listed in Table 13, or variants or fragments thereof.

In some embodiments, the payload antibody has 100% identity to one or more of the antibody polypeptides listed in Table 13, or variants or fragments thereof.

In some embodiments, the payload region of the AAV particle comprises a nucleic acid sequence with at least 50% identity to one or more nucleic acid sequences listed in Table 13, or variants or fragments thereof. The payload nucleic acid sequence may have 50%, 51%, 52%, 53%, 54%, 55%, 56% 57% 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 13, or variants or fragments thereof.

In some embodiments, the payload nucleic acid sequence has 90% identity to one or more of the nucleic acid sequences listed in Table 13, or variants or fragments thereof.

In some embodiments, the payload nucleic acid sequence has 91% identity to one or more of the nucleic acid sequences listed in Table 13, or variants or fragments thereof.

In some embodiments, the payload nucleic acid sequence has 92% identity to one or more of the nucleic acid sequences listed in Table 13, or variants or fragments thereof.

In some embodiments, the payload nucleic acid sequence has 93% identity to one or more of the nucleic acid sequences listed in Table 13, or variants or fragments thereof.

In some embodiments, the payload nucleic acid sequence has 94% identity to one or more of the nucleic acid sequences listed in Table 13, or variants or fragments thereof.

In some embodiments, the payload nucleic acid sequence has 95% identity to one or more of the nucleic acid sequences listed in Table 13, or variants or fragments thereof.

In some embodiments, the payload nucleic acid sequence has 96% identity to one or more of the nucleic acid sequences listed in Table 13, or variants or fragments thereof.

In some embodiments, the payload nucleic acid sequence has 97% identity to one or more of the nucleic acid sequences listed in Table 13, or variants or fragments thereof.

In some embodiments, the payload nucleic acid sequence has 98% identity to one or more of the nucleic acid sequences listed in Table 13, or variants or fragments thereof.

In some embodiments, the payload nucleic acid sequence has 99% identity to one or more of the nucleic acid sequences listed in Table 13, or variants or fragments thereof.

In some embodiments, the payload nucleic acid sequence has 100% identity to one or more of the nucleic acid sequences listed in Table 13, or variants or fragments thereof.

In some embodiments, the payload antibody may be variants of any of the antibody polypeptides listed in Table 13, that exclude one or more amino acids designated as “X” or “x” in the described polypeptide sequence, wherein X may represent any amino acid. In some embodiments, the payload nucleic acid sequence may be variants of any of the nucleic acid sequences listed in Table 13, that exclude one or more nucleic acids designated as “n” or “N” in the described nucleic acid sequence, wherein n may represent any nucleic acid.

TABLE 13
Pain antibodies

| | | Type | | SEQ |
|---|---|---|---|---|
| Ab ID | Component | (PRT/DNA) | Reference | ID NO |
| PN1 | CDR | DNA | US20180142038A1; SEQ ID NO: 14 | 8717 |
| PN2 | CDR | DNA | US20180142038A1; SEQ ID NO: 198 | 8718 |
| PN3 | CDR | DNA | US20180142038A1; SEQ ID NO: 174 | 8719 |
| PN4 | CDR | DNA | US20180142038A1; SEQ ID NO: 134 | 8729 |
| PN5 | CDR | DNA | U.S. 10/202,450; SEQ ID NO: 17 | 8721 |
| PN6 | CDR | DNA | U.S. 10/202,450; SEQ ID NO: 15 | 8722 |
| PN7 | CDR | DNA | US20180142038A1; SEQ ID NO: 158 | 8723 |
| PN8 | CDR | DNA | US20180142038A1; SEQ ID NO: 118 | 8724 |
| PN9 | CDR | DNA | US20180142038A1; SEQ ID NO: 38 | 8725 |
| PN10 | CDR | DNA | US20160244743A1; SEQ ID NO: 13 | 8726 |
| PN11 | CDR | DNA | US20160244743A1; SEQ ID NO: 3 | 8727 |
| PN12 | CDR | DNA | US20160244743A1; SEQ ID NO: 11 | 8728 |
| PN13 | CDR | DNA | US20160244743A1; SEQ ID NO: 14 | 8729 |
| PN14 | CDR | DNA | US20160244743A1; SEQ ID NO: 8 | 8730 |
| PN15 | CDR | DNA | US20160244743A1; SEQ ID NO: 2 | 8731 |
| PN16 | CDR | DNA | US20160244743A1; SEQ ID NO: 6 | 8732 |
| PN17 | CDR | DNA | US20160244743A1; SEQ ID NO: 10 | 8733 |
| PN18 | CDR | DNA | US20160244743A1; SEQ ID NO: 5 | 8734 |
| PN19 | CDR | DNA | US20160244743A1; SEQ ID NO: 9 | 8735 |
| PN20 | CDR | DNA | US20160244743A1; SEQ ID NO: 12 | 8736 |
| PN21 | CDR | DNA | US20160244743A1; SEQ ID NO: 7 | 8737 |
| PN22 | CDR | DNA | US20160244743A1; SEQ ID NO: 4 | 8738 |
| PN23 | CDR | DNA | U.S. Pat. No. 8,986,694; SEQ ID NO: 112 | 8739 |
| PN24 | CDR | DNA | US20180142038A1; SEQ ID NO: 194 | 8740 |
| PN25 | CDR | DNA | US20180142038A1; SEQ ID NO: 34 | 8741 |
| PN26 | CDR | DNA | US20180142038A1; SEQ ID NO: 78 | 8742 |
| PN27 | CDR | DNA | US20180142038A1; SEQ ID NO: 154 | 8743 |
| PN28 | CDR | DNA | US20180142038A1; SEQ ID NO: 74 | 8744 |
| PN29 | CDR | DNA | US20180142038A1; SEQ ID NO: 114 | 8745 |
| PN30 | CDR | DNA | US20160244743A1; SEQ ID NO: 1 | 8746 |
| PN31 | CDR | DNA | US20180142038A1; SEQ ID NO: 76 | 8747 |
| PN32 | CDR | DNA | US20180142038A1; SEQ ID NO: 116 | 8748 |
| PN33 | CDR | DNA | US20180142038A1; SEQ ID NO: 196 | 8749 |
| PN34 | CDR | DNA | US20180142038A1; SEQ ID NO: 98 | 8750 |
| PN35 | CDR | DNA | US20180142038A1; SEQ ID NO: 18 | 8751 |
| PN36 | CDR | DNA | U.S. 10/202,450; SEQ ID NO: 18 | 8752 |
| PN37 | CDR | DNA | U.S. Pat. No. 8,986,694; SEQ ID NO: 39 | 8753 |
| PN38 | CDR | DNA | US20160244743A1; SEQ ID NO: 16 | 8754 |
| PN39 | CDR | DNA | US20160244743A1; SEQ ID NO: 17 | 8755 |
| PN40 | CDR | DNA | US20180142038A1; SEQ ID NO: 54 | 8756 |
| PN41 | CDR | DNA | U.S. 10/202,450; SEQ ID NO: 13 | 8757 |
| PN42 | CDR | DNA | US20180142038A1; SEQ ID NO: 178 | 8758 |
| PN43 | CDR | DNA | US20160244743A1; SEQ ID NO: 15 | 8759 |
| PN44 | CDR | DNA | US20180142038A1; SEQ ID NO: 36 | 8760 |
| PN45 | CDR | DNA | US20180142038A1; SEQ ID NO: 156 | 8761 |
| PN46 | CDR | DNA | US20180142038A1; SEQ ID NO: 138 | 8762 |
| PN47 | CDR | DNA | US20180142038A1; SEQ ID NO: 96 | 8763 |
| PN48 | CDR | DNA | US20180142038A1; SEQ ID NO: 16 | 8764 |
| PN49 | CDR | DNA | US20180142038A1; SEQ ID NO: 176 | 8765 |
| PN50 | CDR | DNA | U.S. 10/202,450; SEQ ID NO: 14 | 8766 |
| PN51 | CDR | DNA | US20180142038A1; SEQ ID NO: 136 | 8767 |
| PN52 | CDR | DNA | US20180142038A1; SEQ ID NO: 56 | 8768 |
| PN53 | CDR | DNA | U.S. 10/202,450; SEQ ID NO: 19 | 8769 |
| PN54 | CDR | DNA | US20160244743A1; SEQ ID NO: 18 | 8770 |
| PN55 | CDR | PRT | U.S. Pat. No. 8,926,977; SEQ ID NO: 27 | 8771 |
| PN56 | CDR | PRT | U.S. Pat. No. 8,926,977; SEQ ID NO: 45 | 8772 |
| PN57 | CDR | PRT | U.S. Pat. No. 8,926,977; SEQ ID NO: 9 | 8773 |
| PN58 | CDR | PRT | U.S. Pat. No. 8,926,977; SEQ ID NO: 48 | 8774 |
| PN59 | CDR | PRT | U.S. Pat. No. 8,926,977; SEQ ID NO: 18 | 8775 |
| PN60 | CDR | PRT | U.S. Pat. No. 8,926,977; SEQ ID NO: 54 | 8776 |
| PN61 | CDR | PRT | U.S. Pat. No. 8,926,977; SEQ ID NO: 36 | 8777 |
| PN62 | CDR | PRT | U.S. Pat. No. 8,926,977; SEQ ID NO: 26 | 8778 |
| PN63 | CDR | PRT | U.S. Pat. No. 8,926,977; SEQ ID NO: 16 | 8779 |
| PN64 | CDR | PRT | U.S. Pat. No. 8,926,977; SEQ ID NO: 10 | 8780 |
| PN65 | CDR | PRT | U.S. Pat. No. 8,926,977; SEQ ID NO: 59 | 8781 |
| PN66 | CDR | PRT | U.S. Pat. No. 8,926,977; SEQ ID NO: 6 | 8782 |
| PN67 | CDR | PRT | U.S. Pat. No. 8,926,977; SEQ ID NO: 24 | 8783 |
| PN68 | CDR | PRT | U.S. Pat. No. 8,926,977; SEQ ID NO: 12 | 8784 |
| PN69 | CDR | PRT | U.S. Pat. No. 8,926,977; SEQ ID NO: 42 | 8785 |
| PN70 | CDR | PRT | U.S. Pat. No. 8,926,977; SEQ ID NO: 60 | 8786 |
| PN71 | CDR | PRT | U.S. Pat. No. 8,926,977; SEQ ID NO: 5 | 8787 |
| PN72 | CDR | PRT | U.S. Pat. No. 8,926,977; SEQ ID NO: 23 | 8788 |
| PN73 | CDR | PRT | U.S. Pat. No. 8,926,977; SEQ ID NO: 11 | 8789 |
| PN74 | CDR | PRT | U.S. Pat. No. 8,926,977; SEQ ID NO: 29 | 8790 |
| PN75 | CDR | PRT | U.S. Pat. No. 8,926,977; SEQ ID NO: 52 | 8791 |
| PN76 | CDR | PRT | U.S. Pat. No. 8,926,977; SEQ ID NO: 22 | 8792 |
| PN77 | CDR | PRT | U.S. Pat. No. 8,926,977; SEQ ID NO: 39 | 8793 |
| PN78 | CDR | PRT | U.S. Pat. No. 8,926,977; SEQ ID NO: 3 | 8794 |
| PN79 | CDR | PRT | U.S. Pat. No. 8,926,977; SEQ ID NO: 30 | 8795 |
| PN80 | CDR | PRT | U.S. Pat. No. 8,926,977; SEQ ID NO: 35 | 8796 |
| PN81 | CDR | PRT | U.S. Pat. No. 8,926,977; SEQ ID NO: 46 | 8797 |
| PN82 | CDR | PRT | U.S. Pat. No. 8,926,977; SEQ ID NO: 58 | 8798 |
| PN83 | CDR | PRT | U.S. Pat. No. 8,926,977; SEQ ID NO: 31 | 8799 |
| PN84 | CDR | PRT | U.S. Pat. No. 8,926,977; SEQ ID NO: 49 | 8800 |
| PN85 | CDR | PRT | U.S. Pat. No. 8,926,977; SEQ ID NO: 37 | 8801 |
| PN86 | CDR | PRT | U.S. Pat. No. 8,926,977; SEQ ID NO: 57 | 8802 |
| PN87 | CDR | PRT | U.S. Pat. No. 8,926,977; SEQ ID NO: 51 | 8803 |
| PN88 | CDR | PRT | U.S. Pat. No. 8,926,977; SEQ ID NO: 33 | 8804 |
| PN89 | CDR | PRT | U.S. Pat. No. 8,926,977; SEQ ID NO: 43 | 8805 |
| PN90 | CDR | PRT | U.S. Pat. No. 8,926,977; SEQ ID NO: 55 | 8806 |
| PN91 | CDR | PRT | U.S. Pat. No. 8,926,977; SEQ ID NO: 25 | 8807 |
| PN92 | CDR | PRT | U.S. Pat. No. 8,926,977; SEQ ID NO: 7 | 8808 |
| PN93 | CDR | PRT | U.S. Pat. No. 8,926,977; SEQ ID NO: 19 | 8809 |
| PN94 | CDR | PRT | U.S. Pat. No. 8,926,977; SEQ ID NO: 13 | 8810 |
| PN95 | CDR | PRT | U.S. Pat. No. 8,926,977; SEQ ID NO: 1 | 8811 |
| PN96 | CDR | PRT | U.S. Pat. No. 8,926,977; SEQ ID NO: 8 | 8812 |
| PN97 | CDR | PRT | U.S. Pat. No. 8,926,977; SEQ ID NO: 14 | 8813 |
| PN98 | CDR | PRT | U.S. Pat. No. 8,926,977; SEQ ID NO: 50 | 8814 |
| PN99 | CDR | PRT | U.S. Pat. No. 8,926,977; SEQ ID NO: 47 | 8815 |
| PN100 | CDR | PRT | U.S. Pat. No. 8,926,977; SEQ ID NO: 53 | 8816 |
| PN101 | CDR | PRT | U.S. Pat. No. 8,926,977; SEQ ID NO: 34 | 8817 |
| PN102 | CDR | PRT | U.S. Pat. No. 8,926,977; SEQ ID NO: 17 | 8818 |
| PN103 | CDR | PRT | U.S. Pat. No. 8,926,977; SEQ ID NO: 40 | 8819 |
| PN104 | CDR | PRT | U.S. Pat. No. 8,926,977; SEQ ID NO: 28 | 8820 |
| PN105 | CDR | PRT | U.S. Pat. No. 8,926,977; SEQ ID NO: 15 | 8821 |
| PN106 | CDR | PRT | U.S. Pat. No. 8,926,977; SEQ ID NO: 21 | 8822 |
| PN107 | CDR | PRT | U.S. Pat. No. 8,926,977; SEQ ID NO: 41 | 8823 |
| PN108 | CDR | PRT | U.S. Pat. No. 8,926,977; SEQ ID NO: 2 | 8824 |
| PN109 | CDR | PRT | U.S. 10/112,996; SEQ ID NO: 8 | 8825 |
| PN110 | CDR | PRT | US20170306013A1; SEQ ID NO: 486 | 8826 |
| PN111 | CDR | PRT | US20160207996A1; SEQ ID NO: 181 | 8827 |
| PN112 | CDR | PRT | US20160207996A1; SEQ ID NO: 65 | 8828 |
| PN113 | CDR | PRT | US20160207996A1; SEQ ID NO: 146 | 8829 |
| PN114 | CDR | PRT | US20160207996A1; SEQ ID NO: 508 | 8830 |
| PN115 | CDR | PRT | US20160207996A1; SEQ ID NO: 537 | 8831 |
| PN116 | CDR | PRT | US20160207996A1; SEQ ID NO: 52 | 8832 |
| PN117 | CDR | PRT | U.S. 10/112,996; SEQ ID NO: 33 | 8833 |
| PN118 | CDR | PRT | US20160207996A1; SEQ ID NO: 119 | 8834 |
| PN119 | CDR | PRT | US20160207996A1; SEQ ID NO: 482 | 8835 |
| PN120 | CDR | PRT | US20160207996A1; SEQ ID NO: 484 | 8836 |
| PN121 | CDR | PRT | US20160207996A1; SEQ ID NO: 485 | 8837 |
| PN122 | CDR | PRT | US20160207996A1; SEQ ID NO: 16 | 8838 |
| PN123 | CDR | PRT | US20160207996A1; SEQ ID NO: 17 | 8839 |
| PN124 | CDR | PRT | US20170306013A1; SEQ ID NO: 403 | 8840 |
| PN125 | CDR | PRT | US20160207996A1; SEQ ID NO: 206 | 8841 |
| PN126 | CDR | PRT | US20160207996A1; SEQ ID NO: 481 | 8842 |
| PN127 | CDR | PRT | US20160207996A1; SEQ ID NO: 32 | 8843 |
| PN128 | CDR | PRT | US20160207996A1; SEQ ID NO: 569 | 8844 |
| PN129 | CDR | PRT | US20160207996A1; SEQ ID NO: 215 | 8845 |
| PN130 | CDR | PRT | US20160207996A1; SEQ ID NO: 565 | 8846 |
| PN131 | CDR | PRT | US20160207996A1; SEQ ID NO: 214 | 8847 |
| PN132 | CDR | PRT | US20160207996A1; SEQ ID NO: 300 | 8848 |
| PN133 | CDR | PRT | US20160207996A1; SEQ ID NO: 567 | 8849 |
| PN134 | CDR | PRT | US20160207996A1; SEQ ID NO: 207 | 8850 |
| PN135 | CDR | PRT | US20160207996A1; SEQ ID NO: 210 | 8851 |
| PN136 | CDR | PRT | US20160207996A1; SEQ ID NO: 212 | 8852 |
| PN137 | CDR | PRT | US20160207996A1; SEQ ID NO: 211 | 8853 |
| PN138 | CDR | PRT | US20160207996A1; SEQ ID NO: 208 | 8854 |
| PN139 | CDR | PRT | US20160207996A1; SEQ ID NO: 570 | 8855 |
| PN140 | CDR | PRT | US20160207996A1; SEQ ID NO: 568 | 8856 |
| PN141 | CDR | PRT | US20170306013A1; SEQ ID NO: 387 | 8857 |
| PN142 | CDR | PRT | US20170306013A1; SEQ ID NO: 435 | 8858 |
| PN143 | CDR | PRT | US20170306013A1; SEQ ID NO: 355 | 8859 |
| PN144 | CDR | PRT | US20170306013A1; SEQ ID NO: 339 | 8860 |
| PN145 | CDR | PRT | US20160207996A1; SEQ ID NO: 575 | 8861 |
| PN146 | CDR | PRT | US20160207996A1; SEQ ID NO: 574 | 8862 |
| PN147 | CDR | PRT | US20160207996A1; SEQ ID NO: 573 | 8863 |
| PN148 | CDR | PRT | U.S. 10/112,996; SEQ ID NO: 77 | 8864 |
| PN149 | CDR | PRT | US20180142038A1; SEQ ID NO: 46 | 8865 |
| PN150 | CDR | PRT | US20180142038A1; SEQ ID NO: 126 | 8866 |
| PN151 | CDR | PRT | US20180142038A1; SEQ ID NO: 166 | 8867 |
| PN152 | CDR | PRT | US20180142038A1; SEQ ID NO: 6 | 8868 |
| PN153 | CDR | PRT | US20180142038A1; SEQ ID NO: 86 | 8869 |
| PN154 | CDR | PRT | US20140227267A1; SEQ ID NO: 76 | 8870 |
| PN155 | CDR | PRT | US20140227267A1; SEQ ID NO: 68 | 8871 |
| PN156 | CDR | PRT | US20140227267A1; SEQ ID NO: 77 | 8872 |
| PN157 | CDR | PRT | US20140227267A1; SEQ ID NO: 78 | 8873 |
| PN158 | CDR | PRT | US20140227267A1; SEQ ID NO: 80 | 8874 |
| PN159 | CDR | PRT | US20140227267A1; SEQ ID NO: 65 | 8875 |
| PN160 | CDR | PRT | US20140227267A1; SEQ ID NO: 81 | 8876 |
| PN161 | CDR | PRT | US20140227267A1; SEQ ID NO: 99 | 8877 |
| PN162 | CDR | PRT | US20140227267A1; SEQ ID NO: 70 | 8878 |
| PN163 | CDR | PRT | US20140227267A1; SEQ ID NO: 62 | 8879 |
| PN164 | CDR | PRT | US20140227267A1; SEQ ID NO: 100 | 8880 |
| PN165 | CDR | PRT | US20140227267A1; SEQ ID NO: 66 | 8881 |
| PN166 | CDR | PRT | US20140227267A1; SEQ ID NO: 64 | 8882 |
| PN167 | CDR | PRT | US20140227267A1; SEQ ID NO: 82 | 8883 |
| PN168 | CDR | PRT | US20140227267A1; SEQ ID NO: 72 | 8884 |
| PN169 | CDR | PRT | US20140227267A1; SEQ ID NO: 101 | 8885 |
| PN170 | CDR | PRT | US20140227267A1; SEQ ID NO: 83 | 8886 |
| PN171 | CDR | PRT | US20140227267A1; SEQ ID NO: 84 | 8887 |
| PN172 | CDR | PRT | US20140227267A1; SEQ ID NO: 56 | 8888 |
| PN173 | CDR | PRT | US20140227267A1; SEQ ID NO: 55 | 8889 |
| PN174 | CDR | PRT | US20140227267A1; SEQ ID NO: 53 | 8890 |
| PN175 | CDR | PRT | US20140227267A1; SEQ ID NO: 89 | 8891 |
| PN176 | CDR | PRT | US20140227267A1; SEQ ID NO: 73 | 8892 |
| PN177 | CDR | PRT | US20140227267A1; SEQ ID NO: 48 | 8893 |
| PN178 | CDR | PRT | US20140227267A1; SEQ ID NO: 71 | 8894 |
| PN179 | CDR | PRT | US20140227267A1; SEQ ID NO: 59 | 8895 |
| PN180 | CDR | PRT | US20140227267A1; SEQ ID NO: 60 | 8896 |
| PN181 | CDR | PRT | US20140227267A1; SEQ ID NO: 97 | 8897 |
| PN182 | CDR | PRT | US20140227267A1; SEQ ID NO: 102 | 8898 |
| PN183 | CDR | PRT | US20140227267A1; SEQ ID NO: 96 | 8899 |
| PN184 | CDR | PRT | US20140227267A1; SEQ ID NO: 92 | 8900 |
| PN185 | CDR | PRT | US20140227267A1; SEQ ID NO: 58 | 8901 |
| PN186 | CDR | PRT | US20140227267A1; SEQ ID NO: 95 | 8902 |
| PN187 | CDR | PRT | US20140227267A1; SEQ ID NO: 63 | 8903 |
| PN188 | CDR | PRT | US20140227267A1; SEQ ID NO: 87 | 8904 |
| PN189 | CDR | PRT | US20140227267A1; SEQ ID NO: 52 | 8905 |
| PN190 | CDR | PRT | US20140227267A1; SEQ ID NO: 49 | 8906 |
| PN191 | CDR | PRT | US20140227267A1; SEQ ID NO: 79 | 8907 |
| PN192 | CDR | PRT | US20140227267A1; SEQ ID NO: 98 | 8908 |
| PN193 | CDR | PRT | US20140227267A1; SEQ ID NO: 61 | 8909 |
| PN194 | CDR | PRT | US20140227267A1; SEQ ID NO: 94 | 8910 |
| PN195 | CDR | PRT | US20140227267A1; SEQ ID NO: 54 | 8911 |
| PN196 | CDR | PRT | US20140227267A1; SEQ ID NO: 50 | 8912 |
| PN197 | CDR | PRT | US20140227267A1; SEQ ID NO: 69 | 8913 |
| PN198 | CDR | PRT | US20140227267A1; SEQ ID NO: 93 | 8914 |
| PN199 | CDR | PRT | US20140227267A1; SEQ ID NO: 51 | 8915 |
| PN200 | CDR | PRT | US20140227267A1; SEQ ID NO: 85 | 8916 |
| PN201 | CDR | PRT | US20140227267A1; SEQ ID NO: 90 | 8917 |
| PN202 | CDR | PRT | US20140227267A1; SEQ ID NO: 86 | 8918 |
| PN203 | CDR | PRT | US20140227267A1; SEQ ID NO: 91 | 8919 |
| PN204 | CDR | PRT | US20140227267A1; SEQ ID NO: 57 | 8920 |
| PN205 | CDR | PRT | US20140227267A1; SEQ ID NO: 74 | 8921 |
| PN206 | CDR | PRT | US20140227267A1; SEQ ID NO: 75 | 8922 |
| PN207 | CDR | PRT | US20140227267A1; SEQ ID NO: 88 | 8923 |
| PN208 | CDR | PRT | US20140227267A1; SEQ ID NO: 67 | 8924 |
| PN209 | CDR | PRT | US20160207996A1; SEQ ID NO: 502 | 8925 |
| PN210 | CDR | PRT | US20170306013A1; SEQ ID NO: 386 | 8926 |
| PN211 | CDR | PRT | US20160207996A1; SEQ ID NO: 500 | 8927 |
| PN212 | CDR | PRT | US20160207996A1; SEQ ID NO: 128 | 8928 |
| PN213 | CDR | PRT | US20160207996A1; SEQ ID NO: 289 | 8929 |
| PN214 | CDR | PRT | US20160207996A1; SEQ ID NO: 143 | 8930 |
| PN215 | CDR | PRT | US20160207996A1; SEQ ID NO: 34 | 8931 |
| PN216 | CDR | PRT | US20170306013A1; SEQ ID NO: 334 | 8932 |
| PN217 | CDR | PRT | US20170306013A1; SEQ ID NO: 446 | 8933 |
| PN218 | CDR | PRT | US20160207996A1; SEQ ID NO: 463 | 8934 |
| PN219 | CDR | PRT | US20160207996A1; SEQ ID NO: 102 | 8935 |
| PN220 | CDR | PRT | US20160207996A1; SEQ ID NO: 186 | 8936 |
| PN221 | CDR | PRT | U.S. 10/202,450; SEQ ID NO: 10 | 8937 |
| PN222 | CDR | PRT | US20160207996A1; SEQ ID NO: 293 | 8938 |
| PN223 | CDR | PRT | US20170306013A1; SEQ ID NO: 480 | 8939 |
| PN224 | CDR | PRT | US20180142038A1; SEQ ID NO: 48 | 8940 |
| PN225 | CDR | PRT | US20160207996A1; SEQ ID NO: 188 | 8941 |
| PN226 | CDR | PRT | US20160207996A1; SEQ ID NO: 553 | 8942 |
| PN227 | CDR | PRT | U.S. 10/112,996; SEQ ID NO: 62 | 8943 |
| PN228 | CDR | PRT | US20180251740A1; SEQ ID NO: 37 | 8944 |
| PN229 | CDR | PRT | US20160207996A1; SEQ ID NO: 480 | 8945 |
| PN230 | CDR | PRT | U.S. 10/112,996; SEQ ID NO: 35 | 8946 |
| PN231 | CDR | PRT | U.S. 10/112,996; SEQ ID NO: 24 | 8947 |
| PN232 | CDR | PRT | US20160207996A1; SEQ ID NO: 505 | 8948 |
| PN233 | CDR | PRT | US20160207996A1; SEQ ID NO: 109 | 8949 |
| PN234 | CDR | PRT | U.S. 10/112,996; SEQ ID NO: 68 | 8950 |
| PN235 | CDR | PRT | U.S. 10/112,996; SEQ ID NO: 14 | 8951 |
| PN236 | CDR | PRT | US20170306013A1; SEQ ID NO: 434 | 8952 |
| PN237 | CDR | PRT | US20180142038A1; SEQ ID NO: 44 | 8953 |
| PN238 | CDR | PRT | US20160207996A1; SEQ ID NO: 504 | 8954 |
| PN239 | CDR | PRT | U.S. 10/112,996; SEQ ID NO: 20 | 8955 |
| PN240 | CDR | PRT | US20160207996A1; SEQ ID NO: 82 | 8956 |
| PN241 | CDR | PRT | U.S. 10/112,996; SEQ ID NO: 72 | 8957 |
| PN242 | CDR | PRT | US20160207996A1; SEQ ID NO: 548 | 8958 |
| PN243 | CDR | PRT | US20160207996A1; SEQ ID NO: 551 | 8959 |
| PN244 | CDR | PRT | US20160207996A1; SEQ ID NO: 145 | 8960 |
| PN245 | CDR | PRT | U.S. 10/112,996; SEQ ID NO: 60 | 8961 |
| PN246 | CDR | PRT | U.S. 10/202,450; SEQ ID NO: 5 | 8962 |
| PN247 | CDR | PRT | US20170306013A1; SEQ ID NO: 431 | 8963 |
| PN248 | CDR | PRT | US20170306013A1; SEQ ID NO: 354 | 8964 |
| PN249 | CDR | PRT | US20170306013A1; SEQ ID NO: 338 | 8965 |
| PN250 | CDR | PRT | US20160207996A1; SEQ ID NO: 53 | 8966 |
| PN251 | CDR | PRT | US20160207996A1; SEQ ID NO: 538 | 8967 |
| PN252 | CDR | PRT | U.S. 10/112,996; SEQ ID NO: 15 | 8968 |
| PN253 | CDR | PRT | US20170306013A1; SEQ ID NO: 402 | 8969 |
| PN254 | CDR | PRT | US20160207996A1; SEQ ID NO: 51 | 8970 |
| PN255 | CDR | PRT | U.S. 10/112,996; SEQ ID NO: 18 | 8971 |
| PN256 | CDR | PRT | US20170306013A1; SEQ ID NO: 479 | 8972 |
| PN257 | CDR | PRT | US20160207996A1; SEQ ID NO: 161 | 8973 |
| PN258 | CDR | PRT | US20160207996A1; SEQ ID NO: 31 | 8974 |
| PN259 | CDR | PRT | US20180142038A1; SEQ ID NO: 168 | 8975 |
| PN260 | CDR | PRT | US20160207996A1; SEQ ID NO: 144 | 8976 |
| PN261 | CDR | PRT | US20160207996A1; SEQ ID NO: 142 | 8977 |
| PN262 | CDR | PRT | US20180251740A1; SEQ ID NO: 39 | 8978 |
| PN263 | CDR | PRT | US20180251740A1; SEQ ID NO: 40 | 8979 |
| PN264 | CDR | PRT | US20160207996A1; SEQ ID NO: 33 | 8980 |
| PN265 | CDR | PRT | US20160207996A1; SEQ ID NO: 163 | 8981 |
| PN266 | CDR | PRT | US20160207996A1; SEQ ID NO: 552 | 8982 |
| PN267 | CDR | PRT | US20160207996A1; SEQ ID NO: 29 | 8983 |
| PN268 | CDR | PRT | US20160207996A1; SEQ ID NO: 28 | 8984 |
| PN269 | CDR | PRT | US20160207996A1; SEQ ID NO: 149 | 8985 |
| PN270 | CDR | PRT | US20160207996A1; SEQ ID NO: 483 | 8986 |
| PN271 | CDR | PRT | US20160207996A1; SEQ ID NO: 147 | 8987 |
| PN272 | CDR | PRT | US20160207996A1; SEQ ID NO: 555 | 8988 |
| PN273 | CDR | PRT | US20170306013A1; SEQ ID NO: 383 | 8989 |
| PN274 | CDR | PRT | U.S. 10/112,996; SEQ ID NO: 6 | 8990 |
| PN275 | CDR | PRT | US20170306013A1; SEQ ID NO: 335 | 8991 |
| PN276 | CDR | PRT | US20170306013A1; SEQ ID NO: 415 | 8992 |
| PN277 | CDR | PRT | US20160207996A1; SEQ ID NO: 107 | 8993 |
| PN278 | CDR | PRT | US20170306013A1; SEQ ID NO: 482 | 8994 |
| PN279 | CDR | PRT | US20160207996A1; SEQ ID NO: 62 | 8995 |
| PN280 | CDR | PRT | US20160207996A1; SEQ ID NO: 509 | 8996 |
| PN281 | CDR | PRT | U.S. 10/112,996; SEQ ID NO: 23 | 8997 |
| PN282 | CDR | PRT | U.S. 10/112,996; SEQ ID NO: 17 | 8998 |
| PN283 | CDR | PRT | US20160207996A1; SEQ ID NO: 120 | 8999 |
| PN284 | CDR | PRT | US20170306013A1; SEQ ID NO: 337 | 9000 |
| PN285 | CDR | PRT | U.S. 10/112,996; SEQ ID NO: 59 | 9001 |
| PN286 | CDR | PRT | U.S. 10/112,996; SEQ ID NO: 71 | 9002 |
| PN287 | CDR | PRT | U.S. 10/112,996; SEQ ID NO: 65 | 9003 |
| PN288 | CDR | PRT | US20170306013A1; SEQ ID NO: 401 | 9004 |
| PN289 | CDR | PRT | US20170306013A1; SEQ ID NO: 417 | 9005 |
| PN290 | CDR | PRT | US20170306013A1; SEQ ID NO: 433 | 9006 |
| PN291 | CDR | PRT | U.S. 10/202,450; SEQ ID NO: 9 | 9007 |
| PN292 | CDR | PRT | US20170306013A1; SEQ ID NO: 385 | 9008 |
| PN293 | CDR | PRT | US20170306013A1; SEQ ID NO: 481 | 9009 |
| PN294 | CDR | PRT | US20160207996A1; SEQ ID NO: 168 | 9010 |
| PN295 | CDR | PRT | US20160207996A1; SEQ ID NO: 172 | 9011 |
| PN296 | CDR | PRT | US20160207996A1; SEQ ID NO: 167 | 9012 |
| PN297 | CDR | PRT | US20160207996A1; SEQ ID NO: 164 | 9013 |
| PN298 | CDR | PRT | US20160207996A1; SEQ ID NO: 165 | 9014 |
| PN299 | CDR | PRT | US20160207996A1; SEQ ID NO: 531 | 9015 |
| PN300 | CDR | PRT | US20160207996A1; SEQ ID NO: 534 | 9016 |
| PN301 | CDR | PRT | US20160207996A1; SEQ ID NO: 533 | 9017 |
| PN302 | CDR | PRT | U.S. 10/112,996; SEQ ID NO: 32 | 9018 |
| PN303 | CDR | PRT | U.S. 10/112,996; SEQ ID NO: 2 | 9019 |
| PN304 | CDR | PRT | US20160207996A1; SEQ ID NO: 67 | 9020 |
| PN305 | CDR | PRT | US20160207996A1; SEQ ID NO: 54 | 9021 |
| PN306 | CDR | PRT | US20160207996A1; SEQ ID NO: 47 | 9022 |
| PN307 | CDR | PRT | US20160207996A1; SEQ ID NO: 48 | 9023 |
| PN308 | CDR | PRT | US20160207996A1; SEQ ID NO: 46 | 9024 |
| PN309 | CDR | PRT | US20160207996A1; SEQ ID NO: 530 | 9025 |
| PN310 | CDR | PRT | US20160207996A1; SEQ ID NO: 169 | 9026 |
| PN311 | CDR | PRT | US20160207996A1; SEQ ID NO: 170 | 9027 |
| PN312 | CDR | PRT | US20170306013A1; SEQ ID NO: 406 | 9028 |
| PN313 | CDR | PRT | US20170306013A1; SEQ ID NO: 502 | 9029 |
| PN314 | CDR | PRT | US20160207996A1; SEQ ID NO: 457 | 9030 |
| PN315 | CDR | PRT | US20180142038A1; SEQ ID NO: 106 | 9031 |
| PN316 | CDR | PRT | US20180142038A1; SEQ ID NO: 66 | 9032 |
| PN317 | CDR | PRT | US20180142038A1; SEQ ID NO: 186 | 9033 |
| PN318 | CDR | PRT | U.S. 10/112,996; SEQ ID NO: 21 | 9034 |
| PN319 | CDR | PRT | U.S. 10/112,996; SEQ ID NO: 27 | 9035 |
| PN320 | CDR | PRT | US20170306013A1; SEQ ID NO: 534 | 9036 |
| PN321 | CDR | PRT | US20170306013A1; SEQ ID NO: 374 | 9037 |
| PN322 | CDR | PRT | US20160207996A1; SEQ ID NO: 477 | 9038 |
| PN323 | CDR | PRT | US20160207996A1; SEQ ID NO: 462 | 9039 |
| PN324 | CDR | PRT | US20170306013A1; SEQ ID NO: 504 | 9040 |
| PN325 | CDR | PRT | U.S. 10/202,450; SEQ ID NO: 11 | 9041 |
| PN326 | CDR | PRT | US20170306013A1; SEQ ID NO: 376 | 9042 |
| PN327 | CDR | PRT | US20170306013A1; SEQ ID NO: 344 | 9043 |
| PN328 | CDR | PRT | US20170306013A1; SEQ ID NO: 408 | 9044 |
| PN329 | CDR | PRT | US20170306013A1; SEQ ID NO: 552 | 9045 |
| PN330 | CDR | PRT | US20160207996A1; SEQ ID NO: 511 | 9046 |
| PN331 | CDR | PRT | US20160207996A1; SEQ ID NO: 455 | 9047 |
| PN332 | CDR | PRT | U.S. 10/112,996; SEQ ID NO: 29 | 9048 |
| PN333 | CDR | PRT | US20160207996A1; SEQ ID NO: 100 | 9049 |
| PN334 | CDR | PRT | U.S. Pat. No. 8,986,694; SEQ ID NO: 115 | 9050 |
| PN335 | CDR | PRT | US20160207996A1; SEQ ID NO: 12 | 9051 |
| PN336 | CDR | PRT | US20180142038A1; SEQ ID NO: 4 | 9052 |
| PN337 | CDR | PRT | US20160207996A1; SEQ ID NO: 465 | 9053 |
| PN338 | CDR | PRT | US20160207996A1; SEQ ID NO: 458 | 9054 |
| PN339 | CDR | PRT | US20160207996A1; SEQ ID NO: 11 | 9055 |
| PN340 | CDR | PRT | US20160207996A1; SEQ ID NO: 13 | 9056 |
| PN341 | CDR | PRT | US20160207996A1; SEQ ID NO: 506 | 9057 |
| PN342 | CDR | PRT | US20180142038A1; SEQ ID NO: 184 | 9058 |
| PN343 | CDR | PRT | U.S. 10/112,996; SEQ ID NO: 1 | 9059 |
| PN344 | CDR | PRT | U.S. 10/112,996; SEQ ID NO: 7 | 9060 |
| PN345 | CDR | PRT | U.S. 10/112,996; SEQ ID NO: 31 | 9061 |
| PN346 | CDR | PRT | US20180142038A1; SEQ ID NO: 24 | 9062 |
| PN347 | CDR | PRT | US20160207996A1; SEQ ID NO: 162 | 9063 |
| PN348 | CDR | PRT | U.S. 10/112,996; SEQ ID NO: 55 | 9064 |
| PN349 | CDR | PRT | U.S. 10/112,996; SEQ ID NO: 19 | 9065 |
| PN350 | CDR | PRT | U.S. 10/112,996; SEQ ID NO: 73 | 9066 |
| PN351 | CDR | PRT | US20160207996A1; SEQ ID NO: 213 | 9067 |
| PN352 | CDR | PRT | U.S. 10/202,450; SEQ ID NO: 7 | 9068 |
| PN353 | CDR | PRT | US20170306013A1; SEQ ID NO: 389 | 9069 |
| PN354 | CDR | PRT | US20170306013A1; SEQ ID NO: 421 | 9070 |
| PN355 | CDR | PRT | US20180142038A1; SEQ ID NO: 28 | 9071 |
| PN356 | CDR | PRT | US20180142038A1; SEQ ID NO: 108 | 9072 |
| PN357 | CDR | PRT | US20180142038A1; SEQ ID NO: 68 | 9073 |
| PN358 | CDR | PRT | U.S. 10/112,996; SEQ ID NO: 63 | 9074 |
| PN359 | CDR | PRT | U.S. 10/112,996; SEQ ID NO: 69 | 9075 |
| PN360 | CDR | PRT | U.S. 10/112,996; SEQ ID NO: 57 | 9076 |
| PN361 | CDR | PRT | US20160207996A1; SEQ ID NO: 166 | 9077 |
| PN362 | CDR | PRT | US20160207996A1; SEQ ID NO: 535 | 9078 |
| PN363 | CDR | PRT | US20170306013A1; SEQ ID NO: 485 | 9079 |
| PN364 | CDR | PRT | US20180142038A1; SEQ ID NO: 148 | 9080 |
| PN365 | CDR | PRT | US20160207996A1; SEQ ID NO: 49 | 9081 |
| PN366 | CDR | PRT | US20160207996A1; SEQ ID NO: 50 | 9082 |
| PN367 | CDR | PRT | US20160207996A1; SEQ ID NO: 564 | 9083 |
| PN368 | CDR | PRT | US20160207996A1; SEQ ID NO: 77 | 9084 |
| PN369 | CDR | PRT | US20160207996A1; SEQ ID NO: 78 | 9085 |
| PN370 | CDR | PRT | U.S. 10/112,996; SEQ ID NO: 3 | 9086 |
| PN371 | CDR | PRT | US20160207996A1; SEQ ID NO: 84 | 9087 |
| PN372 | CDR | PRT | U.S. 10/112,996; SEQ ID NO: 9 | 9088 |
| PN373 | CDR | PRT | U.S. Pat. No. 8,986,694; SEQ ID NO: 113 | 9089 |
| PN374 | CDR | PRT | US20160207996A1; SEQ ID NO: 563 | 9090 |
| PN375 | CDR | PRT | US20170306013A1; SEQ ID NO: 488 | 9091 |
| PN376 | CDR | PRT | US20170306013A1; SEQ ID NO: 392 | 9092 |
| PN377 | CDR | PRT | US20160207996A1; SEQ ID NO: 205 | 9093 |
| PN378 | CDR | PRT | US20160207996A1; SEQ ID NO: 80 | 9094 |
| PN379 | CDR | PRT | US20160207996A1; SEQ ID NO: 571 | 9095 |
| PN380 | CDR | PRT | US20160207996A1; SEQ ID NO: 79 | 9096 |
| PN381 | CDR | PRT | US20170306013A1; SEQ ID NO: 501 | 9097 |
| PN382 | CDR | PRT | US20170306013A1; SEQ ID NO: 469 | 9098 |
| PN383 | CDR | PRT | US20170306013A1; SEQ ID NO: 341 | 9099 |
| PN384 | CDR | PRT | US20170306013A1; SEQ ID NO: 373 | 9100 |
| PN385 | CDR | PRT | US20170306013A1; SEQ ID NO: 405 | 9101 |
| PN386 | CDR | PRT | US20170306013A1; SEQ ID NO: 437 | 9102 |
| PN387 | CDR | PRT | US20160207996A1; SEQ ID NO: 566 | 9103 |
| PN388 | CDR | PRT | US20160207996A1; SEQ ID NO: 209 | 9104 |
| PN389 | CDR | PRT | U.S. 10/112,996; SEQ ID NO: 13 | 9105 |
| PN390 | CDR | PRT | US20180142038A1; SEQ ID NO: 64 | 9106 |
| PN391 | CDR | PRT | US20180142038A1; SEQ ID NO: 104 | 9107 |
| PN392 | CDR | PRT | US20180142038A1; SEQ ID NO: 144 | 9108 |
| PN393 | CDR | PRT | U.S. 10/112,996; SEQ ID NO: 25 | 9109 |
| PN394 | CDR | PRT | US20160207996A1; SEQ ID NO: 576 | 9110 |
| PN395 | CDR | PRT | U.S. 10/112,996; SEQ ID NO: 75 | 9111 |
| PN396 | CDR | PRT | US20160207996A1; SEQ ID NO: 192 | 9112 |
| PN397 | CDR | PRT | US20160207996A1; SEQ ID NO: 189 | 9113 |
| PN398 | CDR | PRT | US20160207996A1; SEQ ID NO: 187 | 9114 |
| PN399 | CDR | PRT | US20160207996A1; SEQ ID NO: 550 | 9115 |
| PN400 | CDR | PRT | US20160207996A1; SEQ ID NO: 549 | 9116 |
| PN401 | CDR | PRT | US20160207996A1; SEQ ID NO: 191 | 9117 |
| PN402 | CDR | PRT | US20160207996A1; SEQ ID NO: 185 | 9118 |
| PN403 | CDR | PRT | US20160207996A1; SEQ ID NO: 81 | 9119 |
| PN404 | CDR | PRT | US20160207996A1; SEQ ID NO: 204 | 9120 |
| PN405 | CDR | PRT | US20160207996A1; SEQ ID NO: 83 | 9121 |
| PN406 | CDR | PRT | US20170306013A1; SEQ ID NO: 388 | 9122 |
| PN407 | CDR | PRT | US20170306013A1; SEQ ID NO: 420 | 9123 |
| PN408 | CDR | PRT | US20170306013A1; SEQ ID NO: 484 | 9124 |
| PN409 | CDR | PRT | US20160207996A1; SEQ ID NO: 64 | 9125 |
| PN410 | CDR | PRT | US20180251740A1; SEQ ID NO: 35 | 9126 |
| PN411 | CDR | PRT | US20170306013A1; SEQ ID NO: 399 | 9127 |
| PN412 | CDR | PRT | US20160207996A1; SEQ ID NO: 532 | 9128 |
| PN413 | CDR | PRT | US20160207996A1; SEQ ID NO: 501 | 9129 |
| PN414 | CDR | PRT | US20160207996A1; SEQ ID NO: 124 | 9130 |
| PN415 | CDR | PRT | US20160207996A1; SEQ ID NO: 126 | 9131 |
| PN416 | CDR | PRT | US20160207996A1; SEQ ID NO: 127 | 9132 |
| PN417 | CDR | PRT | US20160207996A1; SEQ ID NO: 473 | 9133 |
| PN418 | CDR | PRT | US20160207996A1; SEQ ID NO: 122 | 9134 |
| PN419 | CDR | PRT | US20160207996A1; SEQ ID NO: 121 | 9135 |
| PN420 | CDR | PRT | US20160207996A1; SEQ ID NO: 123 | 9136 |
| PN421 | CDR | PRT | US20160207996A1; SEQ ID NO: 475 | 9137 |
| PN422 | CDR | PRT | US20160207996A1; SEQ ID NO: 476 | 9138 |
| PN423 | CDR | PRT | US20170306013A1; SEQ ID NO: 416 | 9139 |
| PN424 | CDR | PRT | US20170306013A1; SEQ ID NO: 438 | 9140 |
| PN425 | CDR | PRT | US20170306013A1; SEQ ID NO: 384 | 9141 |
| PN426 | CDR | PRT | US20160207996A1; SEQ ID NO: 474 | 9142 |
| PN427 | CDR | PRT | U.S. 10/112,996; SEQ ID NO: 22 | 9143 |
| PN428 | CDR | PRT | U.S. 10/112,996; SEQ ID NO: 4 | 9144 |
| PN429 | CDR | PRT | US20160207996A1; SEQ ID NO: 30 | 9145 |
| PN430 | CDR | PRT | US20170306013A1; SEQ ID NO: 500 | 9146 |
| PN431 | CDR | PRT | US20170306013A1; SEQ ID NO: 468 | 9147 |
| PN432 | CDR | PRT | US20170306013A1; SEQ ID NO: 340 | 9148 |
| PN433 | CDR | PRT | US20170306013A1; SEQ ID NO: 372 | 9149 |
| PN434 | CDR | PRT | US20170306013A1; SEQ ID NO: 404 | 9150 |
| PN435 | CDR | PRT | US20170306013A1; SEQ ID NO: 436 | 9151 |
| PN436 | CDR | PRT | US20170306013A1; SEQ ID NO: 548 | 9152 |
| PN437 | CDR | PRT | US20160207996A1; SEQ ID NO: 461 | 9153 |
| PN438 | CDR | PRT | US20160207996A1; SEQ ID NO: 184 | 9154 |
| PN439 | CDR | PRT | US20160207996A1; SEQ ID NO: 453 | 9155 |
| PN440 | CDR | PRT | US20160207996A1; SEQ ID NO: 182 | 9156 |
| PN441 | CDR | PRT | US20160207996A1; SEQ ID NO: 108 | 9157 |
| PN442 | CDR | PRT | US20160207996A1; SEQ ID NO: 103 | 9158 |
| PN443 | CDR | PRT | US20180142038A1; SEQ ID NO: 128 | 9159 |
| PN444 | CDR | PRT | US20160207996A1; SEQ ID NO: 510 | 9160 |
| PN445 | CDR | PRT | US20160207996A1; SEQ ID NO: 452 | 9161 |
| PN446 | CDR | PRT | U.S. 10/112,996; SEQ ID NO: 36 | 9162 |
| PN447 | CDR | PRT | US20180142038A1; SEQ ID NO: 124 | 9163 |
| PN448 | CDR | PRT | US20160207996A1; SEQ ID NO: 478 | 9164 |
| PN449 | CDR | PRT | US20170306013A1; SEQ ID NO: 432 | 9165 |
| PN450 | CDR | PRT | US20160207996A1; SEQ ID NO: 507 | 9166 |
| PN451 | CDR | PRT | US20160207996A1; SEQ ID NO: 105 | 9167 |
| PN452 | CDR | PRT | US20170306013A1; SEQ ID NO: 478 | 9168 |
| PN453 | CDR | PRT | US20160207996A1; SEQ ID NO: 572 | 9169 |
| PN454 | CDR | PRT | US20160207996A1; SEQ ID NO: 104 | 9170 |
| PN455 | CDR | PRT | US20160207996A1; SEQ ID NO: 456 | 9171 |
| PN456 | CDR | PRT | U.S. 10/112,996; SEQ ID NO: 76 | 9172 |
| PN457 | CDR | PRT | US20160207996A1; SEQ ID NO: 66 | 9173 |
| PN458 | CDR | PRT | U.S. 10/112,996; SEQ ID NO: 34 | 9174 |
| PN459 | CDR | PRT | US20160207996A1; SEQ ID NO: 464 | 9175 |
| PN460 | CDR | PRT | US20160207996A1; SEQ ID NO: 8 | 9176 |
| PN461 | CDR | PRT | US20180142038A1; SEQ ID NO: 188 | 9177 |
| PN462 | CDR | PRT | US20170306013A1; SEQ ID NO: 382 | 9178 |
| PN463 | CDR | PRT | US20160207996A1; SEQ ID NO: 536 | 9179 |
| PN464 | CDR | PRT | US20160207996A1; SEQ ID NO: 148 | 9180 |
| PN465 | CDR | PRT | US20170306013A1; SEQ ID NO: 398 | 9181 |
| PN466 | CDR | PRT | US20160207996A1; SEQ ID NO: 10 | 9182 |
| PN467 | CDR | PRT | US20160207996A1; SEQ ID NO: 101 | 9183 |
| PN468 | CDR | PRT | US20160207996A1; SEQ ID NO: 183 | 9184 |
| PN469 | CDR | PRT | US20160207996A1; SEQ ID NO: 472 | 9185 |
| PN470 | CDR | PRT | US20160207996A1; SEQ ID NO: 460 | 9186 |
| PN471 | CDR | PRT | US20170306013A1; SEQ ID NO: 414 | 9187 |
| PN472 | CDR | PRT | US20170306013A1; SEQ ID NO: 430 | 9188 |
| PN473 | CDR | PRT | US20160207996A1; SEQ ID NO: 512 | 9189 |
| PN474 | CDR | PRT | US20160207996A1; SEQ ID NO: 18 | 9190 |
| PN475 | CDR | PRT | US20160207996A1; SEQ ID NO: 19 | 9191 |
| PN476 | CDR | PRT | US20160207996A1; SEQ ID NO: 21 | 9192 |
| PN477 | CDR | PRT | US20160207996A1; SEQ ID NO: 20 | 9193 |
| PN478 | CDR | PRT | US20160207996A1; SEQ ID NO: 291 | 9194 |
| PN479 | CDR | PRT | US20170306013A1; SEQ ID NO: 400 | 9195 |
| PN480 | CDR | PRT | US20160207996A1; SEQ ID NO: 141 | 9196 |
| PN481 | CDR | PRT | U.S. 10/112,996; SEQ ID NO: 78 | 9197 |
| PN482 | CDR | PRT | US20170306013A1; SEQ ID NO: 483 | 9198 |
| PN483 | CDR | PRT | US20170306013A1; SEQ ID NO: 352 | 9199 |
| PN484 | CDR | PRT | US20170306013A1; SEQ ID NO: 592 | 9200 |
| PN485 | CDR | PRT | US20170306013A1; SEQ ID NO: 336 | 9201 |
| PN486 | CDR | PRT | U.S. 10/202,450; SEQ ID NO: 6 | 9202 |
| PN487 | CDR | PRT | US20160207996A1; SEQ ID NO: 503 | 9203 |
| PN488 | CDR | PRT | US20160207996A1; SEQ ID NO: 63 | 9204 |
| PN489 | CDR | PRT | US20160207996A1; SEQ ID NO: 547 | 9205 |
| PN490 | CDR | PRT | U.S. 10/112,996; SEQ ID NO: 5 | 9206 |
| PN491 | CDR | PRT | U.S. 10/112,996; SEQ ID NO: 11 | 9207 |
| PN492 | CDR | PRT | US20180142038A1; SEQ ID NO: 26 | 9208 |
| PN493 | CDR | PRT | U.S. 10/112,996; SEQ ID NO: 30 | 9209 |
| PN494 | CDR | PRT | U.S. 10/112,996; SEQ ID NO: 16 | 9210 |
| PN495 | CR | PRT | US20180251740A1; SEQ ID NO: 29 | 9211 |
| PN496 | CR | PRT | US20180251740A1; SEQ ID NO: 30 | 9212 |
| PN497 | CR | PRT | US20180251740A1; SEQ ID NO: 31 | 9213 |
| PN498 | Fc | PRT | US20140227267A1; SEQ ID NO: 26 | 9214 |
| PN499 | Fc | PRT | US20140227267A1; SEQ ID NO: 25 | 9215 |
| PN500 | Fc | PRT | US20140227267A1; SEQ ID NO: 33 | 9216 |
| PN501 | Fc | PRT | US20140227267A1; SEQ ID NO: 29 | 9217 |
| PN502 | Fc | PRT | US20140227267A1; SEQ ID NO: 31 | 9218 |
| PN503 | Fc | PRT | US20140227267A1; SEQ ID NO: 27 | 9219 |
| PN504 | Fc | PRT | US20140227267A1; SEQ ID NO: 21 | 9220 |
| PN505 | Fc | PRT | US20140227267A1; SEQ ID NO: 18 | 9221 |
| PN506 | Fc | PRT | US20140227267A1; SEQ ID NO: 20 | 9222 |
| PN507 | Fc | PRT | US20140227267A1; SEQ ID NO: 19 | 9223 |
| PN508 | Fc | PRT | US20140227267A1; SEQ ID NO: 32 | 9224 |
| PN509 | Fc | PRT | US20140227267A1; SEQ ID NO: 28 | 9225 |
| PN510 | Fc | PRT | US20140227267A1; SEQ ID NO: 30 | 9226 |
| PN511 | Fc | PRT | US20140227267A1; SEQ ID NO: 37 | 9227 |
| PN512 | Fc | PRT | US20140227267A1; SEQ ID NO: 36 | 9228 |
| PN513 | Fc | PRT | US20140227267A1; SEQ ID NO: 35 | 9229 |
| PN514 | Fc | PRT | US20140227267A1; SEQ ID NO: 34 | 9230 |
| PN515 | Fc | PRT | US20140227267A1; SEQ ID NO: 46 | 9231 |
| PN516 | Fc | PRT | US20140227267A1; SEQ ID NO: 47 | 9232 |
| PN517 | Fc | PRT | US20140227267A1; SEQ ID NO: 38 | 9233 |
| PN518 | Fc | PRT | US20140227267A1; SEQ ID NO: 39 | 9234 |
| PN519 | Fc | PRT | US20140227267A1; SEQ ID NO: 40 | 9235 |
| PN520 | Fc | PRT | US20140227267A1; SEQ ID NO: 41 | 9236 |
| PN521 | FR | DNA | U.S. Pat. No. 8,992,927; SEQ ID NO: 114 | 9237 |
| PN522 | FR | DNA | US20180142038A1; SEQ ID NO: 193 | 9238 |
| PN523 | FR | DNA | US20180142038A1; SEQ ID NO: 73 | 9239 |
| PN524 | FR | DNA | US20180142038A1; SEQ ID NO: 173 | 9240 |
| PN525 | FR | DNA | US20180142038A1; SEQ ID NO: 13 | 9241 |
| PN526 | FR | DNA | US20180142038A1; SEQ ID NO: 113 | 9242 |
| PN527 | FR | DNA | US20180142038A1; SEQ ID NO: 133 | 9243 |
| PN528 | FR | DNA | US20180142038A1; SEQ ID NO: 53 | 9244 |
| PN529 | FR | DNA | US20180142038A1; SEQ ID NO: 93 | 9245 |
| PN530 | FR | DNA | US20180142038A1; SEQ ID NO: 17 | 9246 |
| PN531 | FR | DNA | US20180142038A1; SEQ ID NO: 137 | 9247 |
| PN532 | FR | DNA | US20180142038A1; SEQ ID NO: 177 | 9248 |
| PN533 | FR | DNA | US20180142038A1; SEQ ID NO: 97 | 9249 |
| PN534 | FR | DNA | US20180142038A1; SEQ ID NO: 57 | 9250 |
| PN535 | FR | DNA | US20180142038A1; SEQ ID NO: 153 | 9251 |
| PN536 | FR | DNA | US20180142038A1; SEQ ID NO: 33 | 9252 |
| PN537 | FR | DNA | U.S. Pat. No. 8,992,927; SEQ ID NO: 110 | 9253 |
| PN538 | FR | DNA | US20180142038A1; SEQ ID NO: 77 | 9254 |
| PN539 | FR | DNA | US20180142038A1; SEQ ID NO: 37 | 9255 |
| PN540 | FR | DNA | US20180142038A1; SEQ ID NO: 197 | 9256 |
| PN541 | FR | DNA | US20180142038A1; SEQ ID NO: 117 | 9257 |
| PN542 | FR | DNA | US20180142038A1; SEQ ID NO: 157 | 9258 |
| PN543 | FR | DNA | US20180142038A1; SEQ ID NO: 139 | 9259 |
| PN544 | FR | DNA | US20180142038A1; SEQ ID NO: 19 | 9260 |
| PN545 | FR | DNA | US20180142038A1; SEQ ID NO: 179 | 9261 |
| PN546 | FR | DNA | US20180142038A1; SEQ ID NO: 59 | 9262 |
| PN547 | FR | DNA | US20180142038A1; SEQ ID NO: 175 | 9263 |
| PN548 | FR | DNA | US20180142038A1; SEQ ID NO: 55 | 9264 |
| PN549 | FR | DNA | US20180142038A1; SEQ ID NO: 135 | 9265 |
| PN550 | FR | DNA | US20180142038A1; SEQ ID NO: 15 | 9266 |
| PN551 | FR | DNA | US20180142038A1; SEQ ID NO: 95 | 9267 |
| PN552 | FR | DNA | US20180142038A1; SEQ ID NO: 155 | 9268 |
| PN553 | FR | DNA | US20180142038A1; SEQ ID NO: 115 | 9269 |
| PN554 | FR | DNA | US20180142038A1; SEQ ID NO: 195 | 9270 |
| PN555 | FR | DNA | US20180142038A1; SEQ ID NO: 119 | 9271 |
| PN556 | FR | PRT | US20160207996A1; SEQ ID NO: 158 | 9272 |
| PN557 | FR | PRT | US20160207996A1; SEQ ID NO: 159 | 9273 |
| PN558 | FR | PRT | US20160207996A1; SEQ ID NO: 156 | 9274 |
| PN559 | FR | PRT | US20160207996A1; SEQ ID NO: 160 | 9275 |
| PN560 | FR | PRT | US20160207996A1; SEQ ID NO: 154 | 9276 |
| PN561 | FR | PRT | US20160207996A1; SEQ ID NO: 45 | 9277 |
| PN562 | FR | PRT | US20160207996A1; SEQ ID NO: 38 | 9278 |
| PN563 | FR | PRT | US20160207996A1; SEQ ID NO: 39 | 9279 |
| PN564 | FR | PRT | US20160207996A1; SEQ ID NO: 518 | 9280 |
| PN565 | FR | PRT | US20160207996A1; SEQ ID NO: 37 | 9281 |
| PN566 | FR | PRT | US20160207996A1; SEQ ID NO: 523 | 9282 |
| PN567 | FR | PRT | US20180142038A1; SEQ ID NO: 23 | 9283 |
| PN568 | FR | PRT | US20180142038A1; SEQ ID NO: 143 | 9284 |
| PN569 | FR | PRT | US20160207996A1; SEQ ID NO: 520 | 9285 |
| PN570 | FR | PRT | US20160207996A1; SEQ ID NO: 519 | 9286 |
| PN571 | FR | PRT | US20160207996A1; SEQ ID NO: 522 | 9287 |
| PN572 | FR | PRT | U.S. Pat. No. 9,908,942; SEQ ID NO: 3 | 9288 |
| PN573 | FR | PRT | U.S. Pat. No. 9,908,941; SEQ ID NO: 2 | 9289 |
| PN574 | FR | PRT | U.S. Pat. No. 9,908,947; SEQ ID NO: 9 | 9290 |
| PN575 | FR | PRT | U.S. Pat. No. 9,908,944; SEQ ID NO: 5 | 9291 |
| PN576 | FR | PRT | U.S. Pat. No. 9,908,946; SEQ ID NO: 7 | 9292 |
| PN577 | FR | PRT | US20160207996A1; SEQ ID NO: 451 | 9293 |
| PN578 | FR | PRT | US20160207996A1; SEQ ID NO: 449 | 9294 |
| PN579 | FR | PRT | US20160207996A1; SEQ ID NO: 447 | 9295 |
| PN580 | FR | PRT | US20160207996A1; SEQ ID NO: 3 | 9296 |
| PN581 | FR | PRT | US20160207996A1; SEQ ID NO: 90 | 9297 |
| PN582 | FR | PRT | US20160207996A1; SEQ ID NO: 6 | 9298 |
| PN583 | FR | PRT | US20160207996A1; SEQ ID NO: 442 | 9299 |
| PN584 | FR | PRT | U.S. Pat. No. 9,908,940; SEQ ID NO: 1 | 9300 |
| PN585 | FR | PRT | U.S. Pat. No. 9,908,943; SEQ ID NO: 4 | 9301 |
| PN586 | FR | PRT | U.S. Pat. No. 9,908,945; SEQ ID NO: 6 | 9302 |
| PN587 | FR | PRT | US20160207996A1; SEQ ID NO: 7 | 9303 |
| PN588 | FR | PRT | US20160207996A1; SEQ ID NO: 91 | 9304 |
| PN589 | FR | PRT | US20160207996A1; SEQ ID NO: 98 | 9305 |
| PN590 | FR | PRT | US20160207996A1; SEQ ID NO: 443 | 9306 |
| PN591 | FR | PRT | US20160207996A1; SEQ ID NO: 92 | 9307 |
| PN592 | FR | PRT | US20160207996A1; SEQ ID NO: 94 | 9308 |
| PN593 | FR | PRT | US20160207996A1; SEQ ID NO: 97 | 9309 |
| PN594 | FR | PRT | US20160207996A1; SEQ ID NO: 93 | 9310 |
| PN595 | FR | PRT | US20160207996A1; SEQ ID NO: 444 | 9311 |
| PN596 | FR | PRT | US20160207996A1; SEQ ID NO: 446 | 9312 |
| PN597 | FR | PRT | US20160207996A1; SEQ ID NO: 445 | 9313 |
| PN598 | FR | PRT | US20160207996A1; SEQ ID NO: 2 | 9314 |
| PN599 | FR | PRT | US20160207996A1; SEQ ID NO: 220 | 9315 |
| PN600 | FR | PRT | US20160207996A1; SEQ ID NO: 288 | 9316 |
| PN601 | FR | PRT | US20160207996A1; SEQ ID NO: 89 | 9317 |
| PN602 | FR | PRT | US20180142038A1; SEQ ID NO: 109 | 9318 |
| PN603 | FR | PRT | US20160207996A1; SEQ ID NO: 582 | 9319 |
| PN604 | FR | PRT | US20160207996A1; SEQ ID NO: 216 | 9320 |
| PN605 | FR | PRT | US20160207996A1; SEQ ID NO: 217 | 9321 |
| PN606 | FR | PRT | US20160207996A1; SEQ ID NO: 578 | 9322 |
| PN607 | FR | PRT | US20160207996A1; SEQ ID NO: 577 | 9323 |
| PN608 | FR | PRT | US20160207996A1; SEQ ID NO: 85 | 9324 |
| PN609 | FR | PRT | US20160207996A1; SEQ ID NO: 580 | 9325 |
| PN610 | FR | PRT | US20160207996A1; SEQ ID NO: 579 | 9326 |
| PN611 | FR | PRT | US20160207996A1; SEQ ID NO: 74 | 9327 |
| PN612 | FR | PRT | US20160207996A1; SEQ ID NO: 69 | 9328 |
| PN613 | FR | PRT | US20160207996A1; SEQ ID NO: 71 | 9329 |
| PN614 | FR | PRT | US20160207996A1; SEQ ID NO: 560 | 9330 |
| PN615 | FR | PRT | US20160207996A1; SEQ ID NO: 73 | 9331 |
| PN616 | FR | PRT | US20160207996A1; SEQ ID NO: 75 | 9332 |
| PN617 | FR | PRT | US20160207996A1; SEQ ID NO: 557 | 9333 |
| PN618 | FR | PRT | US20160207996A1; SEQ ID NO: 198 | 9334 |
| PN619 | FR | PRT | US20160207996A1; SEQ ID NO: 72 | 9335 |
| PN620 | FR | PRT | US20160207996A1; SEQ ID NO: 562 | 9336 |
| PN621 | FR | PRT | US20160207996A1; SEQ ID NO: 70 | 9337 |
| PN622 | FR | PRT | US20160207996A1; SEQ ID NO: 76 | 9338 |
| PN623 | FR | PRT | US20160207996A1; SEQ ID NO: 556 | 9339 |
| PN624 | FR | PRT | US20160207996A1; SEQ ID NO: 561 | 9340 |
| PN625 | FR | PRT | US20160207996A1; SEQ ID NO: 203 | 9341 |
| PN626 | FR | PRT | US20160207996A1; SEQ ID NO: 202 | 9342 |
| PN627 | FR | PRT | US20160207996A1; SEQ ID NO: 199 | 9343 |
| PN628 | FR | PRT | US20160207996A1; SEQ ID NO: 201 | 9344 |
| PN629 | FR | PRT | US20160207996A1; SEQ ID NO: 558 | 9345 |
| PN630 | FR | PRT | US20160207996A1; SEQ ID NO: 196 | 9346 |
| PN631 | FR | PRT | US20160207996A1; SEQ ID NO: 200 | 9347 |
| PN632 | FR | PRT | US20160207996A1; SEQ ID NO: 197 | 9348 |
| PN633 | FR | PRT | US20160207996A1; SEQ ID NO: 559 | 9349 |
| PN634 | FR | PRT | US20180142038A1; SEQ ID NO: 27 | 9350 |
| PN635 | FR | PRT | US20180142038A1; SEQ ID NO: 67 | 9351 |
| PN636 | FR | PRT | US20180142038A1; SEQ ID NO: 187 | 9352 |
| PN637 | FR | PRT | US20160207996A1; SEQ ID NO: 194 | 9353 |
| PN638 | FR | PRT | US20160207996A1; SEQ ID NO: 195 | 9354 |
| PN639 | FR | PRT | US20180142038A1; SEQ ID NO: 107 | 9355 |
| PN640 | FR | PRT | US20180142038A1; SEQ ID NO: 147 | 9356 |
| PN641 | FR | PRT | US20160207996A1; SEQ ID NO: 493 | 9357 |
| PN642 | FR | PRT | U.S. Pat. No. 8,992,927; SEQ ID NO: 111 | 9358 |
| PN643 | FR | PRT | US20160207996A1; SEQ ID NO: 528 | 9359 |
| PN644 | FR | PRT | US20160207996A1; SEQ ID NO: 41 | 9360 |
| PN645 | FR | PRT | US20180142038A1; SEQ ID NO: 183 | 9361 |
| PN646 | FR | PRT | US20160207996A1; SEQ ID NO: 525 | 9362 |
| PN647 | FR | PRT | US20160207996A1; SEQ ID NO: 529 | 9363 |
| PN648 | FR | PRT | US20160207996A1; SEQ ID NO: 40 | 9364 |
| PN649 | FR | PRT | US20160207996A1; SEQ ID NO: 43 | 9365 |
| PN650 | FR | PRT | US20180142038A1; SEQ ID NO: 3 | 9366 |
| PN651 | FR | PRT | US20160207996A1; SEQ ID NO: 4 | 9367 |
| PN652 | FR | PRT | US20160207996A1; SEQ ID NO: 99 | 9368 |
| PN653 | FR | PRT | US20160207996A1; SEQ ID NO: 157 | 9369 |
| PN654 | FR | PRT | US20180142038A1; SEQ ID NO: 163 | 9370 |
| PN655 | FR | PRT | US20180142038A1; SEQ ID NO: 123 | 9371 |
| PN656 | FR | PRT | US20180142038A1; SEQ ID NO: 43 | 9372 |
| PN657 | FR | PRT | US20180142038A1; SEQ ID NO: 83 | 9373 |
| PN658 | FR | PRT | US20160207996A1; SEQ ID NO: 155 | 9374 |
| PN659 | FR | PRT | US20180142038A1; SEQ ID NO: 63 | 9375 |
| PN660 | FR | PRT | US20180142038A1; SEQ ID NO: 103 | 9376 |
| PN661 | FR | PRT | US20160207996A1; SEQ ID NO: 448 | 9377 |
| PN662 | FR | PRT | US20160207996A1; SEQ ID NO: 5 | 9378 |
| PN663 | FR | PRT | US20160207996A1; SEQ ID NO: 450 | 9379 |
| PN664 | FR | PRT | US20160207996A1; SEQ ID NO: 1 | 9380 |
| PN665 | FR | PRT | US20160207996A1; SEQ ID NO: 219 | 9381 |
| PN666 | FR | PRT | US20160207996A1; SEQ ID NO: 218 | 9382 |
| PN667 | FR | PRT | US20180142038A1; SEQ ID NO: 47 | 9383 |
| PN668 | FR | PRT | US20180142038A1; SEQ ID NO: 87 | 9384 |
| PN669 | FR | PRT | US20180142038A1; SEQ ID NO: 127 | 9385 |
| PN670 | FR | PRT | US20180142038A1; SEQ ID NO: 167 | 9386 |
| PN671 | FR | PRT | US20160207996A1; SEQ ID NO: 494 | 9387 |
| PN672 | FR | PRT | US20160207996A1; SEQ ID NO: 499 | 9388 |
| PN673 | FR | PRT | US20160207996A1; SEQ ID NO: 22 | 9389 |
| PN674 | FR | PRT | US20160207996A1; SEQ ID NO: 498 | 9390 |
| PN675 | FR | PRT | US20160207996A1; SEQ ID NO: 23 | 9391 |
| PN676 | FR | PRT | US20160207996A1; SEQ ID NO: 27 | 9392 |
| PN677 | FR | PRT | US20160207996A1; SEQ ID NO: 486 | 9393 |
| PN678 | FR | PRT | US20160207996A1; SEQ ID NO: 497 | 9394 |
| PN679 | FR | PRT | US20160207996A1; SEQ ID NO: 139 | 9395 |
| PN680 | FR | PRT | US20160207996A1; SEQ ID NO: 193 | 9396 |
| PN681 | FR | PRT | US20180142038A1; SEQ ID NO: 7 | 9397 |
| PN682 | FR | PRT | US20160207996A1; SEQ ID NO: 24 | 9398 |
| PN683 | FR | PRT | US20160207996A1; SEQ ID NO: 496 | 9399 |
| PN684 | FR | PRT | US20160207996A1; SEQ ID NO: 26 | 9400 |
| PN685 | FR | PRT | US20160207996A1; SEQ ID NO: 25 | 9401 |
| PN686 | FR | PRT | US20160207996A1; SEQ ID NO: 131 | 9402 |
| PN687 | FR | PRT | US20160207996A1; SEQ ID NO: 130 | 9403 |
| PN688 | FR | PRT | US20160207996A1; SEQ ID NO: 292 | 9404 |
| PN689 | FR | PRT | US20160207996A1; SEQ ID NO: 492 | 9405 |
| PN690 | FR | PRT | US20160207996A1; SEQ ID NO: 135 | 9406 |
| PN691 | FR | PRT | US20160207996A1; SEQ ID NO: 488 | 9407 |
| PN692 | FR | PRT | US20160207996A1; SEQ ID NO: 487 | 9408 |
| PN693 | FR | PRT | US20160207996A1; SEQ ID NO: 133 | 9409 |
| PN694 | FR | PRT | US20160207996A1; SEQ ID NO: 490 | 9410 |
| PN695 | FR | PRT | US20160207996A1; SEQ ID NO: 138 | 9411 |
| PN696 | FR | PRT | US20160207996A1; SEQ ID NO: 132 | 9412 |
| PN697 | FR | PRT | US20160207996A1; SEQ ID NO: 491 | 9413 |
| PN698 | FR | PRT | US20160207996A1; SEQ ID NO: 489 | 9414 |
| PN699 | FR | PRT | US20160207996A1; SEQ ID NO: 137 | 9415 |
| PN700 | FR | PRT | US20160207996A1; SEQ ID NO: 134 | 9416 |
| PN701 | FR | PRT | US20160207996A1; SEQ ID NO: 136 | 9417 |
| PN702 | FR | PRT | US20160207996A1; SEQ ID NO: 527 | 9418 |
| PN703 | FR | PRT | US20160207996A1; SEQ ID NO: 521 | 9419 |
| PN704 | FR | PRT | US20160207996A1; SEQ ID NO: 526 | 9420 |
| PN705 | FR | PRT | US20160207996A1; SEQ ID NO: 44 | 9421 |
| PN706 | FR | PRT | US20160207996A1; SEQ ID NO: 42 | 9422 |
| PN707 | FR | PRT | US20160207996A1; SEQ ID NO: 153 | 9423 |
| PN708 | FR | PRT | US20160207996A1; SEQ ID NO: 524 | 9424 |
| PN709 | FR | PRT | US20180142038A1; SEQ ID NO: 185 | 9425 |
| PN710 | FR | PRT | US20160207996A1; SEQ ID NO: 544 | 9426 |
| PN711 | FR | PRT | US20160207996A1; SEQ ID NO: 515 | 9427 |
| PN712 | FR | PRT | US20160207996A1; SEQ ID NO: 150 | 9428 |
| PN713 | FR | PRT | US20180142038A1; SEQ ID NO: 49 | 9429 |
| PN714 | FR | PRT | US20160207996A1; SEQ ID NO: 151 | 9430 |
| PN715 | FR | PRT | US20160207996A1; SEQ ID NO: 152 | 9431 |
| PN716 | FR | PRT | US20160207996A1; SEQ ID NO: 514 | 9432 |
| PN717 | FR | PRT | US20160207996A1; SEQ ID NO: 36 | 9433 |
| PN718 | FR | PRT | US20160207996A1; SEQ ID NO: 110 | 9434 |
| PN719 | FR | PRT | US20160207996A1; SEQ ID NO: 542 | 9435 |
| PN720 | FR | PRT | US20160207996A1; SEQ ID NO: 541 | 9436 |
| PN721 | FR | PRT | US20160207996A1; SEQ ID NO: 177 | 9437 |
| PN722 | FR | PRT | US20160207996A1; SEQ ID NO: 179 | 9438 |
| PN723 | FR | PRT | US20160207996A1; SEQ ID NO: 176 | 9439 |
| PN724 | FR | PRT | US20160207996A1; SEQ ID NO: 468 | 9440 |
| PN725 | FR | PRT | US20160207996A1; SEQ ID NO: 180 | 9441 |
| PN726 | FR | PRT | US20160207996A1; SEQ ID NO: 113 | 9442 |
| PN727 | FR | PRT | US20160207996A1; SEQ ID NO: 112 | 9443 |
| PN728 | FR | PRT | US20180142038A1; SEQ ID NO: 125 | 9444 |
| PN729 | FR | PRT | US20160207996A1; SEQ ID NO: 471 | 9445 |
| PN730 | FR | PRT | US20160207996A1; SEQ ID NO: 469 | 9446 |
| PN731 | FR | PRT | US20180142038A1; SEQ ID NO: 5 | 9447 |
| PN732 | FR | PRT | US20180142038A1; SEQ ID NO: 165 | 9448 |
| PN733 | FR | PRT | US20160207996A1; SEQ ID NO: 470 | 9449 |
| PN734 | FR | PRT | US20160207996A1; SEQ ID NO: 466 | 9450 |
| PN735 | FR | PRT | US20160207996A1; SEQ ID NO: 115 | 9451 |
| PN736 | FR | PRT | US20160207996A1; SEQ ID NO: 467 | 9452 |
| PN737 | FR | PRT | US20160207996A1; SEQ ID NO: 58 | 9453 |
| PN738 | FR | PRT | US20160207996A1; SEQ ID NO: 174 | 9454 |
| PN739 | FR | PRT | US20160207996A1; SEQ ID NO: 543 | 9455 |
| PN740 | FR | PRT | US20160207996A1; SEQ ID NO: 178 | 9456 |
| PN741 | FR | PRT | US20160207996A1; SEQ ID NO: 540 | 9457 |
| PN742 | FR | PRT | US20180142038A1; SEQ ID NO: 145 | 9458 |
| PN743 | FR | PRT | US20160207996A1; SEQ ID NO: 60 | 9459 |
| PN744 | FR | PRT | US20160207996A1; SEQ ID NO: 173 | 9460 |
| PN745 | FR | PRT | US20160207996A1; SEQ ID NO: 546 | 9461 |
| PN746 | FR | PRT | US20160207996A1; SEQ ID NO: 59 | 9462 |
| PN747 | FR | PRT | US20160207996A1; SEQ ID NO: 545 | 9463 |
| PN748 | FR | PRT | US20160207996A1; SEQ ID NO: 56 | 9464 |
| PN749 | FR | PRT | US20160207996A1; SEQ ID NO: 57 | 9465 |
| PN750 | FR | PRT | US20160207996A1; SEQ ID NO: 61 | 9466 |
| PN751 | FR | PRT | US20160207996A1; SEQ ID NO: 539 | 9467 |
| PN752 | FR | PRT | US20160207996A1; SEQ ID NO: 55 | 9468 |
| PN753 | FR | PRT | US20160207996A1; SEQ ID NO: 175 | 9469 |
| PN754 | Full antibody | DNA | US20140227267A1; SEQ ID NO: 3 | 9470 |
| PN755 | Full antibody | DNA | US20140227267A1; SEQ ID NO: 4 | 9471 |
| PN756 | Full antibody | DNA | US20140227267A1; SEQ ID NO: 5 | 9472 |
| PN757 | Full antibody | DNA | US20140227267A1; SEQ ID NO: 6 | 9473 |
| PN758 | Full antibody | DNA | US20140227267A1; SEQ ID NO: 7 | 9474 |
| PN759 | Full antibody | PRT | US20140227267A1; SEQ ID NO: 11 | 9475 |
| PN760 | Full antibody | PRT | US20140227267A1; SEQ ID NO: 10 | 9476 |
| PN761 | Full antibody | PRT | US20140227267A1; SEQ ID NO: 13 | 9477 |
| PN762 | Full antibody | PRT | US20140227267A1; SEQ ID NO: 12 | 9478 |
| PN763 | Full antibody | PRT | US20140227267A1; SEQ ID NO: 14 | 9479 |
| PN764 | HC | DNA | U.S. Pat. No. 8,926,977; SEQ ID NO: 98 | 9480 |
| PN765 | HC | DNA | U.S. Pat. No. 8,926,977; SEQ ID NO: 82 | 9481 |
| PN766 | HC | DNA | U.S. Pat. No. 8,926,977; SEQ ID NO: 74 | 9482 |
| PN767 | HC | DNA | U.S. Pat. No. 8,926,977; SEQ ID NO: 66 | 9483 |
| PN768 | HC | DNA | U.S. Pat. No. 8,926,977; SEQ ID NO: 70 | 9484 |
| PN769 | HC | DNA | U.S. Pat. No. 8,926,977; SEQ ID NO: 90 | 9485 |
| PN770 | HC | DNA | U.S. Pat. No. 8,926,977; SEQ ID NO: 94 | 9486 |
| PN771 | HC | DNA | U.S. Pat. No. 8,926,977; SEQ ID NO: 62 | 9487 |
| PN772 | HC | DNA | U.S. Pat. No. 8,926,977; SEQ ID NO: 78 | 9488 |
| PN773 | HC | DNA | U.S. Pat. No. 8,926,977; SEQ ID NO: 86 | 9489 |
| PN774 | HC | DNA | U.S. Pat. No. 8,986,694; SEQ ID NO: 90 | 9490 |
| PN775 | HC | DNA | US20180142038A1; SEQ ID NO: 171 | 9491 |
| PN776 | HC | DNA | U.S. 10/112,996; SEQ ID NO: 170 | 9492 |
| PN777 | HC | DNA | US20180142038A1; SEQ ID NO: 11 | 9493 |
| PN778 | HC | DNA | US20170306013A1; SEQ ID NO: 458 | 9494 |
| PN779 | HC | DNA | US20140227267A1; SEQ ID NO: 2 | 9495 |
| PN780 | HC | DNA | US20170306013A1; SEQ ID NO: 490 | 9496 |
| PN781 | HC | DNA | US20170306013A1; SEQ ID NO: 346 | 9497 |
| PN782 | HC | DNA | US20170306013A1; SEQ ID NO: 362 | 9498 |
| PN783 | HC | DNA | US20170306013A1; SEQ ID NO: 586 | 9499 |
| PN784 | HC | DNA | US20170306013A1; SEQ ID NO: 538 | 9500 |
| PN785 | HC | DNA | US20170306013A1; SEQ ID NO: 554 | 9501 |
| PN786 | HC | DNA | US20170306013A1; SEQ ID NO: 442 | 9502 |
| PN787 | HC | DNA | US20170306013A1; SEQ ID NO: 506 | 9503 |
| PN788 | HC | DNA | US20170306013A1; SEQ ID NO: 474 | 9504 |
| PN789 | HC | DNA | U.S. Pat. No. 8,986,694; SEQ ID NO: 47 | 9505 |
| PN790 | HC | DNA | US20170306013A1; SEQ ID NO: 378 | 9506 |
| PN791 | HC | DNA | U.S. Pat. No. 8,986,694; SEQ ID NO: 45 | 9507 |
| PN792 | HC | DNA | US20170306013A1; SEQ ID NO: 410 | 9508 |
| PN793 | HC | DNA | U.S. 10/112,996; SEQ ID NO: 142 | 9509 |
| PN794 | HC | DNA | U.S. 10/112,996; SEQ ID NO: 162 | 9510 |
| PN795 | HC | DNA | U.S. 10/112,996; SEQ ID NO: 158 | 9511 |
| PN796 | HC | DNA | U.S. 10/112,996; SEQ ID NO: 138 | 9512 |
| PN797 | HC | DNA | U.S. 10/112,996; SEQ ID NO: 166 | 9513 |
| PN798 | HC | DNA | U.S. 10/112,996; SEQ ID NO: 122 | 9514 |
| PN799 | HC | DNA | U.S. 10/112,996; SEQ ID NO: 130 | 9515 |
| PN800 | HC | DNA | U.S. 10/112,996; SEQ ID NO: 126 | 9516 |
| PN801 | HC | DNA | U.S. 10/112,996; SEQ ID NO: 134 | 9517 |
| PN802 | HC | DNA | US20180142038A1; SEQ ID NO: 131 | 9518 |
| PN803 | HC | DNA | US20180142038A1; SEQ ID NO: 51 | 9519 |
| PN804 | HC | DNA | US20180142038A1; SEQ ID NO: 91 | 9520 |
| PN805 | HC | DNA | U.S. Pat. No. 8,986,694; SEQ ID NO: 84 | 9521 |
| PN806 | HC | DNA | US20170306013A1; SEQ ID NO: 394 | 9522 |
| PN807 | HC | DNA | US20170306013A1; SEQ ID NO: 426 | 9523 |
| PN808 | HC | DNA | U.S. Pat. No. 8,986,694; SEQ ID NO: 86 | 9524 |
| PN809 | HC | DNA | US20180142038A1; SEQ ID NO: 202 | 9525 |
| PN810 | HC | DNA | U.S. Pat. No. 8,986,694; SEQ ID NO: 41 | 9526 |
| PN811 | HC | DNA | U.S. Pat. No. 8,986,694; SEQ ID NO: 43 | 9527 |
| PN812 | HC | DNA | US20180142038A1; SEQ ID NO: 100 | 9528 |
| PN813 | HC | DNA | U.S. Pat. No. 8,986,694; SEQ ID NO: 88 | 9529 |
| PN814 | HC | PRT | U.S. Pat. No. 8,926,977; SEQ ID NO: 100 | 9530 |
| PN815 | HC | PRT | U.S. Pat. No. 8,926,977; SEQ ID NO: 76 | 9531 |
| PN816 | HC | PRT | U.S. Pat. No. 8,926,977; SEQ ID NO: 84 | 9532 |
| PN817 | HC | PRT | U.S. Pat. No. 8,926,977; SEQ ID NO: 68 | 9533 |
| PN818 | HC | PRT | U.S. Pat. No. 8,926,977; SEQ ID NO: 72 | 9534 |
| PN819 | HC | PRT | U.S. Pat. No. 8,926,977; SEQ ID NO: 92 | 9535 |
| PN820 | HC | PRT | U.S. Pat. No. 8,926,977; SEQ ID NO: 96 | 9536 |
| PN821 | HC | PRT | U.S. Pat. No. 8,926,977; SEQ ID NO: 64 | 9537 |
| PN822 | HC | PRT | U.S. Pat. No. 8,926,977; SEQ ID NO: 88 | 9538 |
| PN823 | HC | PRT | U.S. Pat. No. 8,926,977; SEQ ID NO: 80 | 9539 |
| PN824 | HC | PRT | US20170306013A1; SEQ ID NO: 330 | 9540 |
| PN825 | HC | PRT | U.S. Pat. No. 8,986,694; SEQ ID NO: 65 | 9541 |
| PN826 | HC | PRT | US20170306013A1; SEQ ID NO: 602 | 9542 |
| PN827 | HC | PRT | U.S. Pat. No. 8,992,927; SEQ ID NO: 73 | 9543 |
| PN828 | HC | PRT | US20180142038A1; SEQ ID NO: 201 | 9544 |
| PN829 | HC | PRT | U.S. Pat. No. 8,986,694; SEQ ID NO: 61 | 9545 |
| PN830 | HC | PRT | U.S. Pat. No. 8,986,694; SEQ ID NO: 89 | 9546 |
| PN831 | HC | PRT | US20170306013A1; SEQ ID NO: 395 | 9547 |
| PN832 | HC | PRT | U.S. Pat. No. 8,992,927; SEQ ID NO: 71 | 9548 |
| PN833 | HC | PRT | US20170306013A1; SEQ ID NO: 427 | 9549 |
| PN834 | HC | PRT | U.S. Pat. No. 8,986,694; SEQ ID NO: 106 | 9550 |
| PN835 | HC | PRT | U.S. Pat. No. 8,986,694; SEQ ID NO: 85 | 9551 |
| PN836 | HC | PRT | U.S. Pat. No. 8,986,694; SEQ ID NO: 104 | 9552 |
| PN837 | HC | PRT | US20180142038A1; SEQ ID NO: 10 | 9553 |
| PN838 | HC | PRT | U.S. 10/112,996; SEQ ID NO: 172 | 9554 |
| PN839 | HC | PRT | US20180142038A1; SEQ ID NO: 1 | 9555 |
| PN840 | HC | PRT | US20180142038A1; SEQ ID NO: 161 | 9556 |
| PN841 | HC | PRT | US20180142038A1; SEQ ID NO: 121 | 9557 |
| PN842 | HC | PRT | US20180142038A1; SEQ ID NO: 41 | 9558 |
| PN843 | HC | PRT | US20180142038A1; SEQ ID NO: 81 | 9559 |
| PN844 | HC | PRT | U.S. 10/112,996; SEQ ID NO: 164 | 9560 |
| PN845 | HC | PRT | U.S. 10/112,996; SEQ ID NO: 160 | 9561 |
| PN846 | HC | PRT | U.S. 10/112,996; SEQ ID NO: 140 | 9562 |
| PN847 | HC | PRT | U.S. 10/112,996; SEQ ID NO: 136 | 9563 |
| PN848 | HC | PRT | U.S. 10/112,996; SEQ ID NO: 128 | 9564 |
| PN849 | HC | PRT | U.S. 10/112,996; SEQ ID NO: 168 | 9565 |
| PN850 | HC | PRT | U.S. 10/112,996; SEQ ID NO: 132 | 9566 |
| PN851 | HC | PRT | U.S. 10/112,996; SEQ ID NO: 124 | 9567 |
| PN852 | HC | PRT | U.S. 10/112,996; SEQ ID NO: 144 | 9568 |
| PN853 | HC | PRT | US20170306013A1; SEQ ID NO: 507 | 9569 |
| PN854 | HC | PRT | US20170306013A1; SEQ ID NO: 475 | 9570 |
| PN855 | HC | PRT | U.S. 10/072,076; SEQ ID NO: 864 | 9571 |
| PN856 | HC | PRT | US20140227267A1; SEQ ID NO: 9 | 9572 |
| PN857 | HC | PRT | US20170306013A1; SEQ ID NO: 379 | 9573 |
| PN858 | HC | PRT | US20170306013A1; SEQ ID NO: 459 | 9574 |
| PN859 | HC | PRT | US20170306013A1; SEQ ID NO: 347 | 9575 |
| PN860 | HC | PRT | US20170306013A1; SEQ ID NO: 587 | 9576 |
| PN861 | HC | PRT | US20170306013A1; SEQ ID NO: 331 | 9577 |
| PN862 | HC | PRT | US20170306013A1; SEQ ID NO: 363 | 9578 |
| PN863 | HC | PRT | US20170306013A1; SEQ ID NO: 555 | 9579 |
| PN864 | HC | PRT | US20170306013A1; SEQ ID NO: 443 | 9580 |
| PN865 | HC | PRT | US20170306013A1; SEQ ID NO: 411 | 9581 |
| PN866 | HC | PRT | US20170306013A1; SEQ ID NO: 491 | 9582 |
| PN867 | HC | PRT | U.S. Pat. No. 8,992,927; SEQ ID NO: 60 | 9583 |
| PN868 | HC | PRT | U.S. Pat. No. 8,986,694; SEQ ID NO: 91 | 9584 |
| PN869 | LC | DNA | U.S. Pat. No. 8,926,977; SEQ ID NO: 81 | 9585 |
| PN870 | LC | DNA | U.S. Pat. No. 8,926,977; SEQ ID NO: 89 | 9586 |
| PN871 | LC | DNA | U.S. Pat. No. 8,926,977; SEQ ID NO: 61 | 9587 |
| PN872 | LC | DNA | U.S. Pat. No. 8,926,977; SEQ ID NO: 69 | 9588 |
| PN873 | LC | DNA | U.S. Pat. No. 8,926,977; SEQ ID NO: 73 | 9589 |
| PN874 | LC | DNA | U.S. Pat. No. 8,926,977; SEQ ID NO: 85 | 9590 |
| PN875 | LC | DNA | U.S. Pat. No. 8,926,977; SEQ ID NO: 77 | 9591 |
| PN876 | LC | DNA | U.S. Pat. No. 8,926,977; SEQ ID NO: 93 | 9592 |
| PN877 | LC | DNA | U.S. Pat. No. 8,926,977; SEQ ID NO: 97 | 9593 |
| PN878 | LC | DNA | U.S. Pat. No. 8,926,977; SEQ ID NO: 65 | 9594 |
| PN879 | LC | DNA | US20180142038A1; SEQ ID NO: 191 | 9595 |
| PN880 | LC | DNA | U.S. Pat. No. 8,986,694; SEQ ID NO: 55 | 9596 |
| PN881 | LC | DNA | U.S. Pat. No. 8,986,694; SEQ ID NO: 53 | 9597 |
| PN882 | LC | DNA | US20180142038A1; SEQ ID NO: 71 | 9598 |
| PN883 | LC | DNA | US20140227267A1; SEQ ID NO: 1 | 9599 |
| PN884 | LC | DNA | US20180142038A1; SEQ ID NO: 111 | 9600 |
| PN885 | LC | DNA | U.S. Pat. No. 8,986,694; SEQ ID NO: 94 | 9601 |
| PN886 | LC | DNA | U.S. Pat. No. 8,986,694; SEQ ID NO: 49 | 9602 |
| PN887 | LC | DNA | US20180142038A1; SEQ ID NO: 120 | 9603 |
| PN888 | LC | DNA | US20180142038A1; SEQ ID NO: 204 | 9604 |
| PN889 | LC | DNA | U.S. Pat. No. 8,986,694; SEQ ID NO: 98 | 9605 |
| PN890 | LC | DNA | US20170306013A1; SEQ ID NO: 476 | 9606 |
| PN891 | LC | DNA | US20170306013A1; SEQ ID NO: 412 | 9607 |
| PN892 | LC | DNA | US20170306013A1; SEQ ID NO: 380 | 9608 |
| PN893 | LC | DNA | U.S. Pat. No. 8,986,694; SEQ ID NO: 96 | 9609 |
| PN894 | LC | DNA | U.S. 10/112,996; SEQ ID NO: 133 | 9610 |
| PN895 | LC | DNA | US20180142038A1; SEQ ID NO: 151 | 9611 |
| PN896 | LC | DNA | US20180142038A1; SEQ ID NO: 31 | 9612 |
| PN897 | LC | DNA | US20170306013A1; SEQ ID NO: 492 | 9613 |
| PN898 | LC | DNA | US20170306013A1; SEQ ID NO: 428 | 9614 |
| PN899 | LC | DNA | US20170306013A1; SEQ ID NO: 364 | 9615 |
| PN900 | LC | DNA | US20170306013A1; SEQ ID NO: 348 | 9616 |
| PN901 | LC | DNA | US20170306013A1; SEQ ID NO: 556 | 9617 |
| PN902 | LC | DNA | US20170306013A1; SEQ ID NO: 588 | 9618 |
| PN903 | LC | DNA | US20170306013A1; SEQ ID NO: 460 | 9619 |
| PN904 | LC | DNA | US20170306013A1; SEQ ID NO: 524 | 9620 |
| PN905 | LC | DNA | US20170306013A1; SEQ ID NO: 444 | 9621 |
| PN906 | LC | DNA | US20170306013A1; SEQ ID NO: 540 | 9622 |
| PN907 | LC | DNA | US20170306013A1; SEQ ID NO: 396 | 9623 |
| PN908 | LC | DNA | U.S. 10/112,996; SEQ ID NO: 137 | 9624 |
| PN909 | LC | DNA | U.S. 10/112,996; SEQ ID NO: 141 | 9625 |
| PN910 | LC | DNA | U.S. 10/112,996; SEQ ID NO: 161 | 9626 |
| PN911 | LC | DNA | U.S. 10/112,996; SEQ ID NO: 157 | 9627 |
| PN912 | LC | DNA | U.S. 10/112,996; SEQ ID NO: 165 | 9628 |
| PN913 | LC | DNA | U.S. 10/112,996; SEQ ID NO: 129 | 9629 |
| PN914 | LC | DNA | U.S. 10/112,996; SEQ ID NO: 125 | 9630 |
| PN915 | LC | DNA | U.S. 10/112,996; SEQ ID NO: 121 | 9631 |
| PN916 | LC | DNA | U.S. 10/112,996; SEQ ID NO: 169 | 9632 |
| PN917 | LC | DNA | U.S. Pat. No. 8,986,694; SEQ ID NO: 102 | 9633 |
| PN918 | LC | DNA | U.S. Pat. No. 8,986,694; SEQ ID NO: 51 | 9634 |
| PN919 | LC | DNA | U.S. Pat. No. 8,986,694; SEQ ID NO: 100 | 9635 |
| PN920 | LC | DNA | U.S. Pat. No. 8,986,694; SEQ ID NO: 59 | 9636 |
| PN921 | LC | PRT | U.S. Pat. No. 8,926,977; SEQ ID NO: 79 | 9637 |
| PN922 | LC | PRT | U.S. Pat. No. 8,926,977; SEQ ID NO: 71 | 9638 |
| PN923 | LC | PRT | U.S. Pat. No. 8,926,977; SEQ ID NO: 63 | 9639 |
| PN924 | LC | PRT | U.S. Pat. No. 8,926,977; SEQ ID NO: 75 | 9640 |
| PN925 | LC | PRT | U.S. Pat. No. 8,926,977; SEQ ID NO: 87 | 9641 |
| PN926 | LC | PRT | U.S. Pat. No. 8,926,977; SEQ ID NO: 99 | 9642 |
| PN927 | LC | PRT | U.S. Pat. No. 8,926,977; SEQ ID NO: 91 | 9643 |
| PN928 | LC | PRT | U.S. Pat. No. 8,926,977; SEQ ID NO: 67 | 9644 |
| PN929 | LC | PRT | U.S. Pat. No. 8,926,977; SEQ ID NO: 95 | 9645 |
| PN930 | LC | PRT | U.S. Pat. No. 8,926,977; SEQ ID NO: 83 | 9646 |
| PN931 | LC | PRT | U.S. 10/112,996; SEQ ID NO: 139 | 9647 |
| PN932 | LC | PRT | U.S. 10/112,996; SEQ ID NO: 143 | 9648 |
| PN933 | LC | PRT | U.S. 10/112,996; SEQ ID NO: 171 | 9649 |
| PN934 | LC | PRT | U.S. 10/112,996; SEQ ID NO: 131 | 9650 |
| PN935 | LC | PRT | U.S. 10/112,996; SEQ ID NO: 159 | 9651 |
| PN936 | LC | PRT | U.S. 10/112,996; SEQ ID NO: 167 | 9652 |
| PN937 | LC | PRT | U.S. 10/112,996; SEQ ID NO: 163 | 9653 |
| PN938 | LC | PRT | U.S. 10/112,996; SEQ ID NO: 123 | 9654 |
| PN939 | LC | PRT | U.S. 10/112,996; SEQ ID NO: 127 | 9655 |
| PN940 | LC | PRT | U.S. 10/072,076; SEQ ID NO: 866 | 9656 |
| PN941 | LC | PRT | US20170306013A1; SEQ ID NO: 477 | 9657 |
| PN942 | LC | PRT | US20170306013A1; SEQ ID NO: 413 | 9658 |
| PN943 | LC | PRT | US20170306013A1; SEQ ID NO: 381 | 9659 |
| PN944 | LC | PRT | U.S. Pat. No. 8,986,694; SEQ ID NO: 97 | 9660 |
| PN945 | LC | PRT | U.S. Pat. No. 8,986,694; SEQ ID NO: 105 | 9661 |
| PN946 | LC | PRT | US20170306013A1; SEQ ID NO: 525 | 9662 |
| PN947 | LC | PRT | US20170306013A1; SEQ ID NO: 493 | 9663 |
| PN948 | LC | PRT | US20170306013A1; SEQ ID NO: 461 | 9664 |
| PN949 | LC | PRT | US20170306013A1; SEQ ID NO: 589 | 9665 |
| PN950 | LC | PRT | US20170306013A1; SEQ ID NO: 333 | 9666 |
| PN951 | LC | PRT | US20170306013A1; SEQ ID NO: 365 | 9667 |
| PN952 | LC | PRT | US20170306013A1; SEQ ID NO: 445 | 9668 |
| PN953 | LC | PRT | US20170306013A1; SEQ ID NO: 429 | 9669 |
| PN954 | LC | PRT | U.S. 10/112,996; SEQ ID NO: 135 | 9670 |
| PN955 | LC | PRT | US20120263727A1; SEQ ID NO: 15 | 9671 |
| PN956 | LC | PRT | US20180142038A1; SEQ ID NO: 21 | 9672 |
| PN957 | LC | PRT | US20180142038A1; SEQ ID NO: 141 | 9673 |
| PN958 | LC | PRT | US20170306013A1; SEQ ID NO: 397 | 9674 |
| PN959 | LC | PRT | US20170306013A1; SEQ ID NO: 541 | 9675 |
| PN960 | LC | PRT | U.S. Pat. No. 8,986,694; SEQ ID NO: 107 | 9676 |
| PN961 | LC | PRT | U.S. Pat. No. 8,986,694; SEQ ID NO: 52 | 9677 |
| PN962 | LC | PRT | US20180142038A1; SEQ ID NO: 181 | 9678 |
| PN963 | LC | PRT | U.S. Pat. No. 8,992,927; SEQ ID NO: 103 | 9679 |
| PN964 | LC | PRT | US20180251740A1; SEQ ID NO: 20 | 9680 |
| PN965 | LC | PRT | US20180251740A1; SEQ ID NO: 21 | 9681 |
| PN966 | LC | PRT | US20180251740A1; SEQ ID NO: 24 | 9682 |
| PN967 | LC | PRT | US20180251740A1; SEQ ID NO: 26 | 9683 |
| PN968 | LC | PRT | US20180251740A1; SEQ ID NO: 27 | 9684 |
| PN969 | LC | PRT | US20180251740A1; SEQ ID NO: 28 | 9685 |
| PN970 | LC | PRT | US20180251740A1; SEQ ID NO: 22 | 9686 |
| PN971 | LC | PRT | US20180251740A1; SEQ ID NO: 25 | 9687 |
| PN972 | LC | PRT | U.S. Pat. No. 8,986,694; SEQ ID NO: 95 | 9688 |
| PN973 | LC | PRT | US20140227267A1; SEQ ID NO: 8 | 9689 |
| PN974 | LC | PRT | US20180142038A1; SEQ ID NO: 61 | 9690 |
| PN975 | LC | PRT | US20180142038A1; SEQ ID NO: 101 | 9691 |
| PN976 | LC | PRT | US20180142038A1; SEQ ID NO: 110 | 9692 |
| PN977 | LC | PRT | US20180142038A1; SEQ ID NO: 203 | 9693 |
| PN978 | ScFv | PRT | US20160207996A1; SEQ ID NO: 411 | 9694 |
| PN979 | ScFv | PRT | US20160207996A1; SEQ ID NO: 427 | 9695 |
| PN980 | ScFv | PRT | US20160207996A1; SEQ ID NO: 352 | 9696 |
| PN981 | ScFv | PRT | US20160207996A1; SEQ ID NO: 335 | 9697 |
| PN982 | ScFv | PRT | US20160207996A1; SEQ ID NO: 426 | 9698 |
| PN983 | ScFv | PRT | US20160207996A1; SEQ ID NO: 438 | 9699 |
| PN984 | ScFv | PRT | US20160207996A1; SEQ ID NO: 344 | 9700 |
| PN985 | ScFv | PRT | US20160207996A1; SEQ ID NO: 383 | 9701 |
| PN986 | ScFv | PRT | US20160207996A1; SEQ ID NO: 421 | 9702 |
| PN987 | ScFv | PRT | US20160207996A1; SEQ ID NO: 336 | 9703 |
| PN988 | ScFv | PRT | US20160207996A1; SEQ ID NO: 393 | 9704 |
| PN989 | ScFv | PRT | US20160207996A1; SEQ ID NO: 349 | 9705 |
| PN990 | ScFv | PRT | US20160207996A1; SEQ ID NO: 388 | 9706 |
| PN991 | ScFv | PRT | US20160207996A1; SEQ ID NO: 429 | 9707 |
| PN992 | ScFv | PRT | US20160207996A1; SEQ ID NO: 338 | 9708 |
| PN993 | ScFv | PRT | US20160207996A1; SEQ ID NO: 337 | 9709 |
| PN994 | ScFv | PRT | US20160207996A1; SEQ ID NO: 342 | 9719 |
| PN995 | ScFv | PRT | US20160207996A1; SEQ ID NO: 343 | 9711 |
| PN996 | ScFv | PRT | US20160207996A1; SEQ ID NO: 428 | 9712 |
| PN997 | ScFv | PRT | US20160207996A1; SEQ ID NO: 340 | 9713 |
| PN998 | ScFv | PRT | US20160207996A1; SEQ ID NO: 345 | 9714 |
| PN999 | ScFv | PRT | US20160207996A1; SEQ ID NO: 341 | 9715 |
| PN1000 | ScFv | PRT | US20160207996A1; SEQ ID NO: 415 | 9716 |
| PN1001 | ScFv | PRT | US20160207996A1; SEQ ID NO: 414 | 9717 |
| PN1002 | ScFv | PRT | US20160207996A1; SEQ ID NO: 412 | 9718 |
| PN1003 | ScFv | PRT | US20160207996A1; SEQ ID NO: 416 | 9719 |
| PN1004 | ScFv | PRT | US20160207996A1; SEQ ID NO: 420 | 9720 |
| PN1005 | ScFv | PRT | US20160207996A1; SEQ ID NO: 422 | 9721 |
| PN1006 | ScFv | PRT | US20160207996A1; SEQ ID NO: 425 | 9722 |
| PN1007 | ScFv | PRT | US20160207996A1; SEQ ID NO: 378 | 9723 |
| PN1008 | ScFv | PRT | US20160207996A1; SEQ ID NO: 407 | 9724 |
| PN1009 | ScFv | PRT | US20160207996A1; SEQ ID NO: 373 | 9725 |
| PN1010 | ScFv | PRT | US20160207996A1; SEQ ID NO: 372 | 9726 |
| PN1011 | ScFv | PRT | US20160207996A1; SEQ ID NO: 424 | 9727 |
| PN1012 | ScFv | PRT | US20160207996A1; SEQ ID NO: 423 | 9728 |
| PN1013 | ScFv | PRT | US20160207996A1; SEQ ID NO: 370 | 9729 |
| PN1014 | ScFv | PRT | US20160207996A1; SEQ ID NO: 371 | 9730 |
| PN1015 | ScFv | PRT | US20160207996A1; SEQ ID NO: 374 | 9731 |
| PN1016 | ScFv | PRT | US20160207996A1; SEQ ID NO: 375 | 9732 |
| PN1017 | ScFv | PRT | US20160207996A1; SEQ ID NO: 410 | 9733 |
| PN1018 | ScFv | PRT | US20160207996A1; SEQ ID NO: 350 | 9734 |
| PN1019 | ScFv | PRT | US20160207996A1; SEQ ID NO: 329 | 9735 |
| PN1020 | ScFv | PRT | US20160207996A1; SEQ ID NO: 439 | 9736 |
| PN1021 | ScFv | PRT | US20160207996A1; SEQ ID NO: 434 | 9737 |
| PN1022 | ScFv | PRT | US20160207996A1; SEQ ID NO: 382 | 9738 |
| PN1023 | ScFv | PRT | US20160207996A1; SEQ ID NO: 413 | 9739 |
| PN1024 | ScFv | PRT | US20160207996A1; SEQ ID NO: 354 | 9740 |
| PN1025 | ScFv | PRT | US20160207996A1; SEQ ID NO: 333 | 9741 |
| PN1026 | ScFv | PRT | US20160207996A1; SEQ ID NO: 440 | 9742 |
| PN1027 | ScFv | PRT | US20160207996A1; SEQ ID NO: 351 | 9743 |
| PN1028 | ScFv | PRT | US20160207996A1; SEQ ID NO: 385 | 9744 |
| PN1029 | ScFv | PRT | US20160207996A1; SEQ ID NO: 384 | 9745 |
| PN1030 | ScFv | PRT | US20160207996A1; SEQ ID NO: 391 | 9746 |
| PN1031 | ScFv | PRT | US20160207996A1; SEQ ID NO: 332 | 9747 |
| PN1032 | ScFv | PRT | US20160207996A1; SEQ ID NO: 376 | 9748 |
| PN1033 | ScFv | PRT | US20160207996A1; SEQ ID NO: 401 | 9749 |
| PN1034 | ScFv | PRT | US20160207996A1; SEQ ID NO: 377 | 9750 |
| PN1035 | ScFv | PRT | US20160207996A1; SEQ ID NO: 369 | 9751 |
| PN1036 | ScFv | PRT | US20160207996A1; SEQ ID NO: 409 | 9752 |
| PN1037 | ScFv | PRT | US20160207996A1; SEQ ID NO: 389 | 9753 |
| PN1038 | ScFv | PRT | US20160207996A1; SEQ ID NO: 347 | 9754 |
| PN1039 | ScFv | PRT | US20160207996A1; SEQ ID NO: 392 | 9755 |
| PN1040 | ScFv | PRT | US20160207996A1; SEQ ID NO: 346 | 9756 |
| PN1041 | ScFv | PRT | US20160207996A1; SEQ ID NO: 441 | 9757 |
| PN1042 | ScFv | PRT | US20160207996A1; SEQ ID NO: 435 | 9758 |
| PN1043 | ScFv | PRT | US20160207996A1; SEQ ID NO: 432 | 9759 |
| PN1044 | ScFv | PRT | US20160207996A1; SEQ ID NO: 436 | 9760 |
| PN1045 | ScFv | PRT | US20160207996A1; SEQ ID NO: 387 | 9761 |
| PN1046 | ScFv | PRT | US20160207996A1; SEQ ID NO: 386 | 9762 |
| PN1047 | ScFv | PRT | US20160207996A1; SEQ ID NO: 353 | 9763 |
| PN1048 | ScFv | PRT | US20160207996A1; SEQ ID NO: 348 | 9764 |
| PN1049 | ScFv | PRT | US20160207996A1; SEQ ID NO: 334 | 9765 |
| PN1050 | ScFv | PRT | US20160207996A1; SEQ ID NO: 331 | 9766 |
| PN1051 | ScFv | PRT | US20160207996A1; SEQ ID NO: 390 | 9767 |
| PN1052 | ScFv | PRT | US20160207996A1; SEQ ID NO: 339 | 9768 |
| PN1053 | ScFv | PRT | US20160207996A1; SEQ ID NO: 330 | 9769 |
| PN1054 | ScFv | PRT | US20160207996A1; SEQ ID NO: 418 | 9770 |
| PN1055 | ScFv | PRT | US20160207996A1; SEQ ID NO: 417 | 9771 |
| PN1056 | ScFv | PRT | US20160207996A1; SEQ ID NO: 419 | 9772 |
| PN1057 | ScFv | PRT | US20160207996A1; SEQ ID NO: 379 | 9773 |
| PN1058 | ScFv | PRT | US20160207996A1; SEQ ID NO: 395 | 9774 |
| PN1059 | ScFv | PRT | US20160207996A1; SEQ ID NO: 402 | 9775 |
| PN1060 | ScFv | PRT | US20160207996A1; SEQ ID NO: 406 | 9776 |
| PN1061 | ScFv | PRT | US20160207996A1; SEQ ID NO: 399 | 9777 |
| PN1062 | ScFv | PRT | US20160207996A1; SEQ ID NO: 403 | 9778 |
| PN1063 | ScFv | PRT | US20160207996A1; SEQ ID NO: 404 | 9779 |
| PN1064 | ScFv | PRT | US20160207996A1; SEQ ID NO: 405 | 9780 |
| PN1065 | ScFv | PRT | US20160207996A1; SEQ ID NO: 398 | 9781 |
| PN1066 | ScFv | PRT | US20160207996A1; SEQ ID NO: 396 | 9782 |
| PN1067 | ScFv | PRT | US20160207996A1; SEQ ID NO: 400 | 9783 |
| PN1068 | ScFv | PRT | US20160207996A1; SEQ ID NO: 363 | 9784 |
| PN1069 | ScFv | PRT | US20160207996A1; SEQ ID NO: 361 | 9785 |
| PN1070 | ScFv | PRT | US20160207996A1; SEQ ID NO: 368 | 9786 |
| PN1071 | ScFv | PRT | US20160207996A1; SEQ ID NO: 381 | 9787 |
| PN1072 | ScFv | PRT | US20160207996A1; SEQ ID NO: 408 | 9788 |
| PN1073 | ScFv | PRT | US20160207996A1; SEQ ID NO: 365 | 9789 |
| PN1074 | ScFv | PRT | US20160207996A1; SEQ ID NO: 394 | 9790 |
| PN1075 | ScFv | PRT | US20160207996A1; SEQ ID NO: 366 | 9791 |
| PN1076 | ScFv | PRT | US20160207996A1; SEQ ID NO: 364 | 9792 |
| PN1077 | ScFv | PRT | US20160207996A1; SEQ ID NO: 397 | 9793 |
| PN1078 | ScFv | PRT | US20160207996A1; SEQ ID NO: 367 | 9794 |
| PN1079 | ScFv | PRT | US20160207996A1; SEQ ID NO: 358 | 9795 |
| PN1080 | ScFv | PRT | US20160207996A1; SEQ ID NO: 362 | 9796 |
| PN1081 | ScFv | PRT | US20160207996A1; SEQ ID NO: 359 | 9797 |
| PN1082 | ScFv | PRT | US20160207996A1; SEQ ID NO: 355 | 9798 |
| PN1083 | ScFv | PRT | US20160207996A1; SEQ ID NO: 357 | 9799 |
| PN1084 | ScFv | PRT | US20160207996A1; SEQ ID NO: 356 | 9800 |
| PN1085 | ScFv | PRT | US20160207996A1; SEQ ID NO: 360 | 9801 |
| PN1086 | ScFv | PRT | US20160207996A1; SEQ ID NO: 380 | 9802 |
| PN1087 | VH | DNA | U.S. 10/072,076; SEQ ID NO: 121 | 9803 |
| PN1088 | VH | DNA | U.S. 10/072,076; SEQ ID NO: 441 | 9804 |
| PN1089 | VH | DNA | U.S. 10/072,076; SEQ ID NO: 431 | 9805 |
| PN1090 | VH | DNA | U.S. Pat. No. 8,986,694; SEQ ID NO: 79 | 9806 |
| PN1091 | VH | DNA | U.S. 10/202,450; SEQ ID NO: 12 | 9807 |
| PN1092 | VH | DNA | U.S. Pat. No. 8,986,694; SEQ ID NO: 76 | 9808 |
| PN1093 | VH | DNA | U.S. 10/072,076; SEQ ID NO: 137 | 9809 |
| PN1094 | VH | DNA | U.S. 10/072,076; SEQ ID NO: 237 | 9810 |
| PN1095 | VH | DNA | U.S. 10/072,076; SEQ ID NO: 509 | 9811 |
| PN1096 | VH | DNA | U.S. 10/072,076; SEQ ID NO: 325 | 9812 |
| PN1097 | VH | DNA | U.S. 10/072,076; SEQ ID NO: 133 | 9813 |
| PN1098 | VH | DNA | U.S. 10/072,076; SEQ ID NO: 457 | 9814 |
| PN1099 | VH | DNA | US20180142038A1; SEQ ID NO: 172 | 9815 |
| PN1100 | VH | DNA | U.S. 10/072,076; SEQ ID NO: 704 | 9816 |
| PN1101 | VH | DNA | US20180142038A1; SEQ ID NO: 12 | 9817 |
| PN1102 | VH | DNA | U.S. 10/072,076; SEQ ID NO: 69 | 9818 |
| PN1103 | VH | DNA | U.S. 10/072,076; SEQ ID NO: 863 | 9819 |
| PN1104 | VH | DNA | U.S. 10/072,076; SEQ ID NO: 65 | 9820 |
| PN1105 | VH | DNA | U.S. 10/072,076; SEQ ID NO: 867 | 9821 |
| PN1106 | VH | DNA | U.S. 10/072,076; SEQ ID NO: 742 | 9822 |
| PN1107 | VH | DNA | U.S. 10/072,076; SEQ ID NO: 5 | 9823 |
| PN1108 | VH | DNA | U.S. 10/072,076; SEQ ID NO: 700 | 9824 |
| PN1109 | VH | DNA | U.S. 10/072,076; SEQ ID NO: 313 | 9825 |
| PN1110 | VH | DNA | U.S. 10/072,076; SEQ ID NO: 696 | 9826 |
| PN1111 | VH | DNA | U.S. 10/072,076; SEQ ID NO: 535 | 9827 |
| PN1112 | VH | DNA | U.S. 10/072,076; SEQ ID NO: 381 | 9828 |
| PN1113 | VH | DNA | U.S. 10/072,076; SEQ ID NO: 297 | 9829 |
| PN1114 | VH | DNA | U.S. 10/072,076; SEQ ID NO: 389 | 9830 |
| PN1115 | VH | DNA | U.S. 10/072,076; SEQ ID NO: 501 | 9831 |
| PN1116 | VH | DNA | U.S. 10/072,076; SEQ ID NO: 469 | 9832 |
| PN1117 | VH | DNA | U.S. 10/072,076; SEQ ID NO: 349 | 9833 |
| PN1118 | VH | DNA | U.S. 10/072,076; SEQ ID NO: 453 | 9834 |
| PN1119 | VH | DNA | U.S. 10/072,076; SEQ ID NO: 117 | 9835 |
| PN1120 | VH | DNA | U.S. 10/072,076; SEQ ID NO: 473 | 9836 |
| PN1121 | VH | DNA | U.S. 10/072,076; SEQ ID NO: 513 | 9837 |
| PN1122 | VH | DNA | U.S. 10/072,076; SEQ ID NO: 241 | 9838 |
| PN1123 | VH | DNA | U.S. 10/072,076; SEQ ID NO: 491 | 9839 |
| PN1124 | VH | DNA | U.S. 10/072,076; SEQ ID NO: 543 | 9840 |
| PN1125 | VH | DNA | U.S. 10/072,076; SEQ ID NO: 487 | 9841 |
| PN1126 | VH | DNA | U.S. 10/072,076; SEQ ID NO: 481 | 9842 |
| PN1127 | VH | DNA | U.S. 10/072,076; SEQ ID NO: 285 | 9843 |
| PN1128 | VH | DNA | U.S. 10/072,076; SEQ ID NO: 53 | 9844 |
| PN1129 | VH | DNA | U.S. 10/072,076; SEQ ID NO: 189 | 9845 |
| PN1130 | VH | DNA | U.S. 10/072,076; SEQ ID NO: 421 | 9846 |
| PN1131 | VH | DNA | U.S. 10/072,076; SEQ ID NO: 253 | 9847 |
| PN1132 | VH | DNA | U.S. 10/072,076; SEQ ID NO: 269 | 9848 |
| PN1133 | VH | DNA | U.S. 10/072,076; SEQ ID NO: 525 | 9849 |
| PN1134 | VH | DNA | U.S. 10/072,076; SEQ ID NO: 185 | 9850 |
| PN1135 | VH | DNA | U.S. 10/072,076; SEQ ID NO: 459 | 9851 |
| PN1136 | VH | DNA | U.S. 10/072,076; SEQ ID NO: 425 | 9852 |
| PN1137 | VH | DNA | U.S. 10/072,076; SEQ ID NO: 439 | 9853 |
| PN1138 | VH | DNA | U.S. 10/072,076; SEQ ID NO: 289 | 9854 |
| PN1139 | VH | DNA | U.S. 10/072,076; SEQ ID NO: 45 | 9855 |
| PN1140 | VH | DNA | U.S. 10/072,076; SEQ ID NO: 455 | 9856 |
| PN1141 | VH | DNA | U.S. 10/072,076; SEQ ID NO: 265 | 9857 |
| PN1142 | VH | DNA | U.S. 10/072,076; SEQ ID NO: 33 | 9858 |
| PN1143 | VH | DNA | U.S. 10/072,076; SEQ ID NO: 529 | 9859 |
| PN1144 | VH | DNA | U.S. 10/072,076; SEQ ID NO: 523 | 9860 |
| PN1145 | VH | DNA | U.S. 10/072,076; SEQ ID NO: 417 | 9861 |
| PN1146 | VH | DNA | U.S. 10/072,076; SEQ ID NO: 353 | 9862 |
| PN1147 | VH | DNA | U.S. 10/072,076; SEQ ID NO: 341 | 9863 |
| PN1148 | VH | DNA | U.S. 10/072,076; SEQ ID NO: 369 | 9864 |
| PN1149 | VH | DNA | U.S. 10/072,076; SEQ ID NO: 433 | 9865 |
| PN1150 | VH | DNA | U.S. 10/072,076; SEQ ID NO: 429 | 9866 |
| PN1151 | VH | DNA | U.S. 10/072,076; SEQ ID NO: 309 | 9867 |
| PN1152 | VH | DNA | U.S. 10/072,076; SEQ ID NO: 321 | 9868 |
| PN1153 | VH | DNA | U.S. 10/072,076; SEQ ID NO: 317 | 9869 |
| PN1154 | VH | DNA | U.S. 10/072,076; SEQ ID NO: 451 | 9870 |
| PN1155 | VH | DNA | U.S. 10/072,076; SEQ ID NO: 471 | 9871 |
| PN1156 | VH | DNA | U.S. 10/072,076; SEQ ID NO: 475 | 9872 |
| PN1157 | VH | DNA | U.S. 10/072,076; SEQ ID NO: 427 | 9873 |
| PN1158 | VH | DNA | U.S. 10/072,076; SEQ ID NO: 333 | 9874 |
| PN1159 | VH | DNA | U.S. 10/072,076; SEQ ID NO: 393 | 9875 |
| PN1160 | VH | DNA | U.S. 10/072,076; SEQ ID NO: 357 | 9876 |
| PN1161 | VH | DNA | U.S. 10/072,076; SEQ ID NO: 385 | 9877 |
| PN1162 | VH | DNA | U.S. 10/072,076; SEQ ID NO: 467 | 9878 |
| PN1163 | VH | DNA | U.S. 10/072,076; SEQ ID NO: 293 | 9879 |
| PN1164 | VH | DNA | U.S. 10/072,076; SEQ ID NO: 539 | 9880 |
| PN1165 | VH | DNA | U.S. 10/072,076; SEQ ID NO: 21 | 9881 |
| PN1166 | VH | DNA | U.S. 10/072,076; SEQ ID NO: 261 | 9882 |
| PN1167 | VH | DNA | U.S. 10/072,076; SEQ ID NO: 29 | 9883 |
| PN1168 | VH | DNA | U.S. 10/072,076; SEQ ID NO: 465 | 9884 |
| PN1169 | VH | DNA | U.S. 10/072,076; SEQ ID NO: 477 | 9885 |
| PN1170 | VH | DNA | U.S. 10/072,076; SEQ ID NO: 377 | 9886 |
| PN1171 | VH | DNA | U.S. 10/072,076; SEQ ID NO: 209 | 9887 |
| PN1172 | VH | DNA | U.S. 10/072,076; SEQ ID NO: 503 | 9888 |
| PN1173 | VH | DNA | U.S. 10/072,076; SEQ ID NO: 85 | 9889 |
| PN1174 | VH | DNA | U.S. 10/072,076; SEQ ID NO: 229 | 9890 |
| PN1175 | VH | DNA | U.S. 10/072,076; SEQ ID NO: 493 | 9891 |
| PN1176 | VH | DNA | U.S. 10/072,076; SEQ ID NO: 13 | 9892 |
| PN1177 | VH | DNA | U.S. 10/072,076; SEQ ID NO: 461 | 9893 |
| PN1178 | VH | DNA | U.S. 10/072,076; SEQ ID NO: 517 | 9894 |
| PN1179 | VH | DNA | U.S. 10/072,076; SEQ ID NO: 205 | 9895 |
| PN1180 | VH | DNA | U.S. 10/072,076; SEQ ID NO: 193 | 9896 |
| PN1181 | VH | DNA | U.S. 10/072,076; SEQ ID NO: 758 | 9897 |
| PN1182 | VH | DNA | U.S. 10/072,076; SEQ ID NO: 301 | 9898 |
| PN1183 | VH | DNA | U.S. 10/072,076; SEQ ID NO: 57 | 9899 |
| PN1184 | VH | DNA | U.S. 10/072,076; SEQ ID NO: 519 | 9900 |
| PN1185 | VH | DNA | U.S. 10/072,076; SEQ ID NO: 485 | 9901 |
| PN1186 | VH | DNA | U.S. 10/072,076; SEQ ID NO: 499 | 9902 |
| PN1187 | VH | DNA | U.S. 10/072,076; SEQ ID NO: 249 | 9903 |
| PN1188 | VH | DNA | U.S. 10/072,076; SEQ ID NO: 49 | 9904 |
| PN1189 | VH | DNA | U.S. 10/072,076; SEQ ID NO: 489 | 9905 |
| PN1190 | VH | DNA | U.S. 10/072,076; SEQ ID NO: 479 | 9906 |
| PN1191 | VH | DNA | U.S. 10/072,076; SEQ ID NO: 273 | 9907 |
| PN1192 | VH | DNA | U.S. 10/072,076; SEQ ID NO: 145 | 9908 |
| PN1193 | VH | DNA | U.S. 10/072,076; SEQ ID NO: 721 | 9909 |
| PN1194 | VH | DNA | U.S. 10/072,076; SEQ ID NO: 871 | 9910 |
| PN1195 | VH | DNA | U.S. 10/072,076; SEQ ID NO: 177 | 9911 |
| PN1196 | VH | DNA | U.S. 10/072,076; SEQ ID NO: 213 | 9912 |
| PN1197 | VH | DNA | U.S. 10/072,076; SEQ ID NO: 515 | 9913 |
| PN1198 | VH | DNA | U.S. 10/072,076; SEQ ID NO: 97 | 9914 |
| PN1199 | VH | DNA | U.S. 10/072,076; SEQ ID NO: 329 | 9915 |
| PN1200 | VH | DNA | U.S. 10/072,076; SEQ ID NO: 533 | 9916 |
| PN1201 | VH | DNA | U.S. 10/072,076; SEQ ID NO: 541 | 9917 |
| PN1202 | VH | DNA | U.S. 10/072,076; SEQ ID NO: 73 | 9918 |
| PN1203 | VH | DNA | U.S. 10/072,076; SEQ ID NO: 435 | 9919 |
| PN1204 | VH | DNA | U.S. 10/072,076; SEQ ID NO: 141 | 9920 |
| PN1205 | VH | DNA | U.S. 10/072,076; SEQ ID NO: 113 | 9921 |
| PN1206 | VH | DNA | U.S. 10/072,076; SEQ ID NO: 305 | 9922 |
| PN1207 | VH | DNA | U.S. 10/072,076; SEQ ID NO: 157 | 9923 |
| PN1208 | VH | DNA | U.S. 10/072,076; SEQ ID NO: 165 | 9924 |
| PN1209 | VH | DNA | U.S. 10/072,076; SEQ ID NO: 125 | 9925 |
| PN1210 | VH | DNA | U.S. 10/072,076; SEQ ID NO: 161 | 9926 |
| PN1211 | VH | DNA | U.S. 10/072,076; SEQ ID NO: 93 | 9927 |
| PN1212 | VH | DNA | U.S. 10/072,076; SEQ ID NO: 497 | 9928 |
| PN1213 | VH | DNA | U.S. 10/072,076; SEQ ID NO: 365 | 9929 |
| PN1214 | VH | DNA | U.S. 10/072,076; SEQ ID NO: 726 | 9930 |
| PN1215 | VH | DNA | U.S. 10/072,076; SEQ ID NO: 17 | 9931 |
| PN1216 | VH | DNA | U.S. 10/072,076; SEQ ID NO: 1 | 9932 |
| PN1217 | VH | DNA | US20180142038A1; SEQ ID NO: 132 | 9933 |
| PN1218 | VH | DNA | US20180142038A1; SEQ ID NO: 52 | 9934 |
| PN1219 | VH | DNA | US20180142038A1; SEQ ID NO: 92 | 9935 |
| PN1220 | VH | DNA | U.S. 10/072,076; SEQ ID NO: 337 | 9936 |
| PN1221 | VH | DNA | U.S. 10/072,076; SEQ ID NO: 413 | 9937 |
| PN1222 | VH | DNA | U.S. 10/072,076; SEQ ID NO: 109 | 9938 |
| PN1223 | VH | DNA | U.S. 10/072,076; SEQ ID NO: 25 | 9939 |
| PN1224 | VH | DNA | US20160207996A1; SEQ ID NO: 309 | 9940 |
| PN1225 | VH | DNA | US20160207996A1; SEQ ID NO: 307 | 9941 |
| PN1226 | VH | DNA | U.S. 10/072,076; SEQ ID NO: 105 | 9942 |
| PN1227 | VH | DNA | U.S. 10/072,076; SEQ ID NO: 81 | 9943 |
| PN1228 | VH | DNA | U.S. 10/072,076; SEQ ID NO: 41 | 9944 |
| PN1229 | VH | DNA | U.S. 10/072,076; SEQ ID NO: 225 | 9945 |
| PN1230 | VH | DNA | U.S. 10/072,076; SEQ ID NO: 233 | 9946 |
| PN1231 | VH | DNA | U.S. 10/072,076; SEQ ID NO: 277 | 9947 |
| PN1232 | VH | DNA | U.S. 10/072,076; SEQ ID NO: 37 | 9948 |
| PN1233 | VH | DNA | U.S. 10/072,076; SEQ ID NO: 245 | 9949 |
| PN1234 | VH | DNA | U.S. 10/072,076; SEQ ID NO: 521 | 9950 |
| PN1235 | VH | DNA | U.S. 10/072,076; SEQ ID NO: 153 | 9951 |
| PN1236 | VH | DNA | U.S. 10/072,076; SEQ ID NO: 197 | 9952 |
| PN1237 | VH | DNA | U.S. 10/072,076; SEQ ID NO: 221 | 9953 |
| PN1238 | VH | DNA | U.S. 10/072,076; SEQ ID NO: 397 | 9954 |
| PN1239 | VH | DNA | U.S. 10/072,076; SEQ ID NO: 545 | 9955 |
| PN1240 | VH | DNA | U.S. 10/072,076; SEQ ID NO: 77 | 9956 |
| PN1241 | VH | DNA | U.S. 10/072,076; SEQ ID NO: 61 | 9957 |
| PN1242 | VH | DNA | U.S. 10/072,076; SEQ ID NO: 443 | 9958 |
| PN1243 | VH | DNA | U.S. 10/072,076; SEQ ID NO: 257 | 9959 |
| PN1244 | VH | DNA | U.S. 10/072,076; SEQ ID NO: 531 | 9960 |
| PN1245 | VH | DNA | U.S. 10/072,076; SEQ ID NO: 445 | 9961 |
| PN1246 | VH | DNA | U.S. 10/072,076; SEQ ID NO: 345 | 9962 |
| PN1247 | VH | DNA | U.S. 10/072,076; SEQ ID NO: 401 | 9963 |
| PN1248 | VH | DNA | U.S. 10/072,076; SEQ ID NO: 409 | 9964 |
| PN1249 | VH | DNA | U.S. 10/072,076; SEQ ID NO: 495 | 9965 |
| PN1250 | VH | DNA | U.S. 10/072,076; SEQ ID NO: 405 | 9966 |
| PN1251 | VH | DNA | U.S. 10/072,076; SEQ ID NO: 463 | 9967 |
| PN1252 | VH | DNA | U.S. 10/072,076; SEQ ID NO: 101 | 9968 |
| PN1253 | VH | DNA | U.S. 10/072,076; SEQ ID NO: 790 | 9969 |
| PN1254 | VH | DNA | U.S. 10/072,076; SEQ ID NO: 281 | 9970 |
| PN1255 | VH | DNA | U.S. 10/072,076; SEQ ID NO: 217 | 9971 |
| PN1256 | VH | DNA | U.S. 10/072,076; SEQ ID NO: 527 | 9972 |
| PN1257 | VH | DNA | U.S. 10/072,076; SEQ ID NO: 201 | 9973 |
| PN1258 | VH | DNA | U.S. 10/072,076; SEQ ID NO: 507 | 9974 |
| PN1259 | VH | DNA | U.S. 10/072,076; SEQ ID NO: 859 | 9975 |
| PN1260 | VH | DNA | U.S. 10/072,076; SEQ ID NO: 692 | 9976 |
| PN1261 | VH | DNA | U.S. 10/072,076; SEQ ID NO: 537 | 9977 |
| PN1262 | VH | DNA | U.S. 10/072,076; SEQ ID NO: 684 | 9978 |
| PN1263 | VH | DNA | U.S. 10/072,076; SEQ ID NO: 449 | 9979 |
| PN1264 | VH | DNA | U.S. 10/072,076; SEQ ID NO: 149 | 9980 |
| PN1265 | VH | DNA | U.S. 10/072,076; SEQ ID NO: 129 | 9981 |
| PN1266 | VH | DNA | U.S. 10/072,076; SEQ ID NO: 89 | 9982 |
| PN1267 | VH | DNA | U.S. 10/072,076; SEQ ID NO: 361 | 9983 |
| PN1268 | VH | DNA | U.S. 10/072,076; SEQ ID NO: 505 | 9984 |
| PN1269 | VH | DNA | U.S. 10/072,076; SEQ ID NO: 511 | 9985 |
| PN1270 | VH | DNA | US20160207996A1; SEQ ID NO: 305 | 9986 |
| PN1271 | VH | DNA | U.S. Pat. No. 8,986,694; SEQ ID NO: 74 | 9987 |
| PN1272 | VH | DNA | U.S. 10/072,076; SEQ ID NO: 792 | 9988 |
| PN1273 | VH | PRT | US20160207996A1; SEQ ID NO: 603 | 9989 |
| PN1274 | VH | PRT | US20160207996A1; SEQ ID NO: 604 | 9990 |
| PN1275 | VH | PRT | U.S. Pat. No. 8,986,694; SEQ ID NO: 72 | 9991 |
| PN1276 | VH | PRT | US20160207996A1; SEQ ID NO: 597 | 9992 |
| PN1277 | VH | PRT | US20160207996A1; SEQ ID NO: 602 | 9993 |
| PN1278 | VH | PRT | US20160207996A1; SEQ ID NO: 599 | 9994 |
| PN1279 | VH | PRT | US20160207996A1; SEQ ID NO: 598 | 9995 |
| PN1280 | VH | PRT | US20160207996A1; SEQ ID NO: 601 | 9996 |
| PN1281 | VH | PRT | US20160207996A1; SEQ ID NO: 596 | 9997 |
| PN1282 | VH | PRT | US20160207996A1; SEQ ID NO: 594 | 9998 |
| PN1283 | VH | PRT | U.S. 10/072,076; SEQ ID NO: 512 | 9999 |
| PN1284 | VH | PRT | US20160207996A1; SEQ ID NO: 592 | 10000 |
| PN1285 | VH | PRT | US20160207996A1; SEQ ID NO: 221 | 10001 |
| PN1286 | VH | PRT | US20160207996A1; SEQ ID NO: 237 | 10002 |
| PN1287 | VH | PRT | US20160207996A1; SEQ ID NO: 225 | 10003 |
| PN1288 | VH | PRT | US20160207996A1; SEQ ID NO: 583 | 10004 |
| PN1289 | VH | PRT | U.S. Pat. No. 8,986,694; SEQ ID NO: 69 | 10005 |
| PN1290 | VH | PRT | U.S. 10/072,076; SEQ ID NO: 362 | 10006 |
| PN1291 | VH | PRT | U.S. 10/072,076; SEQ ID NO: 791 | 10007 |
| PN1292 | VH | PRT | U.S. 10/072,076; SEQ ID NO: 90 | 10008 |
| PN1293 | VH | PRT | U.S. 10/072,076; SEQ ID NO: 130 | 10009 |
| PN1294 | VH | PRT | U.S. 10/072,076; SEQ ID NO: 450 | 10010 |
| PN1295 | VH | PRT | US20160207996A1; SEQ ID NO: 246 | 10011 |
| PN1296 | VH | PRT | U.S. 10/072,076; SEQ ID NO: 26 | 10012 |
| PN1297 | VH | PRT | U.S. 10/072,076; SEQ ID NO: 693 | 10013 |
| PN1298 | VH | PRT | U.S. 10/072,076; SEQ ID NO: 538 | 10014 |
| PN1299 | VH | PRT | U.S. 10/072,076; SEQ ID NO: 685 | 10015 |
| PN1300 | VH | PRT | US20160207996A1; SEQ ID NO: 591 | 10016 |
| PN1301 | VH | PRT | U.S. 10/072,076; SEQ ID NO: 860 | 10017 |
| PN1302 | VH | PRT | US20160207996A1; SEQ ID NO: 226 | 10018 |
| PN1303 | VH | PRT | U.S. 10/072,076; SEQ ID NO: 282 | 10019 |
| PN1304 | VH | PRT | U.S. 10/072,076; SEQ ID NO: 506 | 10020 |
| PN1305 | VH | PRT | U.S. 10/072,076; SEQ ID NO: 508 | 10021 |
| PN1306 | VH | PRT | U.S. 10/072,076; SEQ ID NO: 202 | 10022 |
| PN1307 | VH | PRT | U.S. 10/072,076; SEQ ID NO: 528 | 10023 |
| PN1308 | VH | PRT | U.S. 10/072,076; SEQ ID NO: 218 | 10024 |
| PN1309 | VH | PRT | U.S. 10/072,076; SEQ ID NO: 110 | 10025 |
| PN1310 | VH | PRT | U.S. 10/072,076; SEQ ID NO: 102 | 10026 |
| PN1311 | VH | PRT | US20160207996A1; SEQ ID NO: 238 | 10027 |
| PN1312 | VH | PRT | US20160207996A1; SEQ ID NO: 245 | 10028 |
| PN1313 | VH | PRT | US20160207996A1; SEQ ID NO: 588 | 10029 |
| PN1314 | VH | PRT | US20160207996A1; SEQ ID NO: 584 | 10030 |
| PN1315 | VH | PRT | US20160207996A1; SEQ ID NO: 585 | 10031 |
| PN1316 | VH | PRT | US20160207996A1; SEQ ID NO: 587 | 10032 |
| PN1317 | VH | PRT | US20160207996A1; SEQ ID NO: 239 | 10033 |
| PN1318 | VH | PRT | US20160207996A1; SEQ ID NO: 241 | 10034 |
| PN1319 | VH | PRT | US20160207996A1; SEQ ID NO: 244 | 10035 |
| PN1320 | VH | PRT | US20160207996A1; SEQ ID NO: 240 | 10036 |
| PN1321 | VH | PRT | US20160207996A1; SEQ ID NO: 242 | 10037 |
| PN1322 | VH | PRT | US20160207996A1; SEQ ID NO: 586 | 10038 |
| PN1323 | VH | PRT | US20160207996A1; SEQ ID NO: 590 | 10039 |
| PN1324 | VH | PRT | US20160207996A1; SEQ ID NO: 589 | 10040 |
| PN1325 | VH | PRT | U.S. 10/072,076; SEQ ID NO: 546 | 10041 |
| PN1326 | VH | PRT | U.S. 10/072,076; SEQ ID NO: 410 | 10042 |
| PN1327 | VH | PRT | U.S. 10/072,076; SEQ ID NO: 496 | 10043 |
| PN1328 | VH | PRT | U.S. 10/072,076; SEQ ID NO: 398 | 10044 |
| PN1329 | VH | PRT | U.S. 10/072,076; SEQ ID NO: 464 | 10045 |
| PN1330 | VH | PRT | U.S. 10/072,076; SEQ ID NO: 414 | 10046 |
| PN1331 | VH | PRT | U.S. 10/072,076; SEQ ID NO: 402 | 10047 |
| PN1332 | VH | PRT | U.S. 10/072,076; SEQ ID NO: 222 | 10048 |
| PN1333 | VH | PRT | U.S. 10/072,076; SEQ ID NO: 346 | 10049 |
| PN1334 | VH | PRT | U.S. 10/072,076; SEQ ID NO: 446 | 10050 |
| PN1335 | VH | PRT | U.S. 10/072,076; SEQ ID NO: 444 | 10051 |
| PN1336 | VH | PRT | U.S. 10/072,076; SEQ ID NO: 258 | 10052 |
| PN1337 | VH | PRT | U.S. 10/072,076; SEQ ID NO: 338 | 10053 |
| PN1338 | VH | PRT | U.S. 10/072,076; SEQ ID NO: 532 | 10054 |
| PN1339 | VH | PRT | U.S. 10/072,076; SEQ ID NO: 406 | 10055 |
| PN1340 | VH | PRT | U.S. 10/072,076; SEQ ID NO: 78 | 10056 |
| PN1341 | VH | PRT | U.S. 10/072,076; SEQ ID NO: 62 | 10057 |
| PN1342 | VH | PRT | U.S. 10/072,076; SEQ ID NO: 522 | 10058 |
| PN1343 | VH | PRT | U.S. 10/072,076; SEQ ID NO: 226 | 10059 |
| PN1344 | VH | PRT | U.S. 10/072,076; SEQ ID NO: 234 | 10060 |
| PN1345 | VH | PRT | U.S. 10/072,076; SEQ ID NO: 246 | 10061 |
| PN1346 | VH | PRT | U.S. 10/072,076; SEQ ID NO: 38 | 10062 |
| PN1347 | VH | PRT | U.S. 10/072,076; SEQ ID NO: 278 | 10063 |
| PN1348 | VH | PRT | U.S. 10/072,076; SEQ ID NO: 154 | 10064 |
| PN1349 | VH | PRT | U.S. 10/072,076; SEQ ID NO: 198 | 10065 |
| PN1350 | VH | PRT | U.S. 10/072,076; SEQ ID NO: 42 | 10066 |
| PN1351 | VH | PRT | U.S. 10/072,076; SEQ ID NO: 82 | 10067 |
| PN1352 | VH | PRT | U.S. 10/072,076; SEQ ID NO: 106 | 10068 |
| PN1353 | VH | PRT | US20160207996A1; SEQ ID NO: 302 | 10069 |
| PN1354 | VH | PRT | US20160207996A1; SEQ ID NO: 236 | 10070 |
| PN1355 | VH | PRT | US20160207996A1; SEQ ID NO: 609 | 10071 |
| PN1356 | VH | PRT | U.S. 10/072,076; SEQ ID NO: 432 | 10072 |
| PN1357 | VH | PRT | U.S. 10/072,076; SEQ ID NO: 442 | 10073 |
| PN1358 | VH | PRT | U.S. 10/072,076; SEQ ID NO: 122 | 10074 |
| PN1359 | VH | PRT | U.S. 10/202,450; SEQ ID NO: 4 | 10075 |
| PN1360 | VH | PRT | U.S. Pat. No. 8,986,694; SEQ ID NO: 67 | 10076 |
| PN1361 | VH | PRT | U.S. Pat. No. 8,986,694; SEQ ID NO: 78 | 10077 |
| PN1362 | VH | PRT | US20160207996A1; SEQ ID NO: 606 | 10078 |
| PN1363 | VH | PRT | US20160207996A1; SEQ ID NO: 610 | 10079 |
| PN1364 | VH | PRT | US20180142038A1; SEQ ID NO: 2 | 10080 |
| PN1365 | VH | PRT | U.S. 10/072,076; SEQ ID NO: 705 | 10081 |
| PN1366 | VH | PRT | US20160207996A1; SEQ ID NO: 222 | 10082 |
| PN1367 | VH | PRT | US20160207996A1; SEQ ID NO: 247 | 10083 |
| PN1368 | VH | PRT | US20180142038A1; SEQ ID NO: 162 | 10084 |
| PN1369 | VH | PRT | US20180142038A1; SEQ ID NO: 122 | 10085 |
| PN1370 | VH | PRT | US20180142038A1; SEQ ID NO: 42 | 10086 |
| PN1371 | VH | PRT | US20180142038A1; SEQ ID NO: 82 | 10087 |
| PN1372 | VH | PRT | U.S. 10/072,076; SEQ ID NO: 382 | 10088 |
| PN1373 | VH | PRT | U.S. 10/072,076; SEQ ID NO: 536 | 10089 |
| PN1374 | VH | PRT | US20160207996A1; SEQ ID NO: 593 | 10090 |
| PN1375 | VH | PRT | US20160207996A1; SEQ ID NO: 224 | 10091 |
| PN1376 | VH | PRT | US20160207996A1; SEQ ID NO: 223 | 10092 |
| PN1377 | VH | PRT | U.S. 10/072,076; SEQ ID NO: 66 | 10093 |
| PN1378 | VH | PRT | U.S. 10/072,076; SEQ ID NO: 701 | 10094 |
| PN1379 | VH | PRT | U.S. 10/072,076; SEQ ID NO: 314 | 10095 |
| PN1380 | VH | PRT | U.S. 10/072,076; SEQ ID NO: 70 | 10096 |
| PN1381 | VH | PRT | U.S. 10/072,076; SEQ ID NO: 466 | 10097 |
| PN1382 | VH | PRT | U.S. 10/072,076; SEQ ID NO: 30 | 10098 |
| PN1383 | VH | PRT | U.S. 10/072,076; SEQ ID NO: 478 | 10099 |
| PN1384 | VH | PRT | U.S. 10/072,076; SEQ ID NO: 378 | 10100 |
| PN1385 | VH | PRT | U.S. 10/072,076; SEQ ID NO: 134 | 10101 |
| PN1386 | VH | PRT | U.S. 10/072,076; SEQ ID NO: 366 | 10102 |
| PN1387 | VH | PRT | U.S. 10/072,076; SEQ ID NO: 498 | 10103 |
| PN1388 | VH | PRT | U.S. 10/072,076; SEQ ID NO: 94 | 10104 |
| PN1389 | VH | PRT | U.S. 10/072,076; SEQ ID NO: 166 | 10105 |
| PN1390 | VH | PRT | U.S. 10/072,076; SEQ ID NO: 158 | 10106 |
| PN1391 | VH | PRT | U.S. 10/072,076; SEQ ID NO: 162 | 10107 |
| PN1392 | VH | PRT | U.S. 10/072,076; SEQ ID NO: 126 | 10108 |
| PN1393 | VH | PRT | US20160207996A1; SEQ ID NO: 595 | 10109 |
| PN1394 | VH | PRT | U.S. 10/072,076; SEQ ID NO: 330 | 10110 |
| PN1395 | VH | PRT | U.S. 10/072,076; SEQ ID NO: 458 | 10111 |
| PN1396 | VH | PRT | U.S. 10/072,076; SEQ ID NO: 142 | 10112 |
| PN1397 | VH | PRT | U.S. 10/072,076; SEQ ID NO: 436 | 10113 |
| PN1398 | VH | PRT | U.S. 10/072,076; SEQ ID NO: 114 | 10114 |
| PN1399 | VH | PRT | U.S. 10/072,076; SEQ ID NO: 98 | 10115 |
| PN1400 | VH | PRT | U.S. 10/072,076; SEQ ID NO: 306 | 10116 |
| PN1401 | VH | PRT | U.S. 10/072,076; SEQ ID NO: 534 | 10117 |
| PN1402 | VH | PRT | U.S. 10/072,076; SEQ ID NO: 542 | 10118 |
| PN1403 | VH | PRT | U.S. 10/072,076; SEQ ID NO: 74 | 10119 |
| PN1404 | VH | PRT | U.S. 10/072,076; SEQ ID NO: 743 | 10120 |
| PN1405 | VH | PRT | U.S. 10/072,076; SEQ ID NO: 516 | 10121 |
| PN1406 | VH | PRT | U.S. 10/072,076; SEQ ID NO: 504 | 10122 |
| PN1407 | VH | PRT | U.S. 10/072,076; SEQ ID NO: 146 | 10123 |
| PN1408 | VH | PRT | U.S. 10/072,076; SEQ ID NO: 274 | 10124 |
| PN1409 | VH | PRT | U.S. 10/072,076; SEQ ID NO: 494 | 10125 |
| PN1410 | VH | PRT | U.S. 10/072,076; SEQ ID NO: 230 | 10126 |
| PN1411 | VH | PRT | U.S. 10/072,076; SEQ ID NO: 490 | 10127 |
| PN1412 | VH | PRT | U.S. 10/072,076; SEQ ID NO: 722 | 10128 |
| PN1413 | VH | PRT | U.S. 10/072,076; SEQ ID NO: 14 | 10129 |
| PN1414 | VH | PRT | U.S. 10/072,076; SEQ ID NO: 500 | 10130 |
| PN1415 | VH | PRT | U.S. 10/072,076; SEQ ID NO: 486 | 10131 |
| PN1416 | VH | PRT | U.S. 10/072,076; SEQ ID NO: 250 | 10132 |
| PN1417 | VH | PRT | U.S. 10/072,076; SEQ ID NO: 262 | 10133 |
| PN1418 | VH | PRT | U.S. 10/072,076; SEQ ID NO: 194 | 10134 |
| PN1419 | VH | PRT | U.S. 10/072,076; SEQ ID NO: 6 | 10135 |
| PN1420 | VH | PRT | U.S. 10/072,076; SEQ ID NO: 759 | 10136 |
| PN1421 | VH | PRT | U.S. 10/072,076; SEQ ID NO: 480 | 10137 |
| PN1422 | VH | PRT | U.S. 10/072,076; SEQ ID NO: 462 | 10138 |
| PN1423 | VH | PRT | U.S. 10/072,076; SEQ ID NO: 50 | 10139 |
| PN1424 | VH | PRT | U.S. 10/072,076; SEQ ID NO: 520 | 10140 |
| PN1425 | VH | PRT | U.S. 10/072,076; SEQ ID NO: 302 | 10141 |
| PN1426 | VH | PRT | U.S. 10/072,076; SEQ ID NO: 518 | 10142 |
| PN1427 | VH | PRT | U.S. 10/072,076; SEQ ID NO: 178 | 10143 |
| PN1428 | VH | PRT | U.S. 10/072,076; SEQ ID NO: 206 | 10144 |
| PN1429 | VH | PRT | U.S. 10/072,076; SEQ ID NO: 86 | 10145 |
| PN1430 | VH | PRT | U.S. 10/072,076; SEQ ID NO: 210 | 10146 |
| PN1431 | VH | PRT | U.S. 10/072,076; SEQ ID NO: 214 | 10147 |
| PN1432 | VH | PRT | U.S. 10/072,076; SEQ ID NO: 2 | 10148 |
| PN1433 | VH | PRT | U.S. 10/072,076; SEQ ID NO: 18 | 10149 |
| PN1434 | VH | PRT | U.S. 10/072,076; SEQ ID NO: 10 | 10150 |
| PN1435 | VH | PRT | U.S. 10/072,076; SEQ ID NO: 540 | 10151 |
| PN1436 | VH | PRT | U.S. 10/072,076; SEQ ID NO: 474 | 10152 |
| PN1437 | VH | PRT | U.S. 10/072,076; SEQ ID NO: 350 | 10153 |
| PN1438 | VH | PRT | U.S. 10/072,076; SEQ ID NO: 118 | 10154 |
| PN1439 | VH | PRT | U.S. 10/072,076; SEQ ID NO: 454 | 10155 |
| PN1440 | VH | PRT | U.S. 10/072,076; SEQ ID NO: 868 | 10156 |
| PN1441 | VH | PRT | U.S. 10/072,076; SEQ ID NO: 138 | 10157 |
| PN1442 | VH | PRT | U.S. 10/072,076; SEQ ID NO: 370 | 10158 |
| PN1443 | VH | PRT | U.S. 10/072,076; SEQ ID NO: 358 | 10159 |
| PN1444 | VH | PRT | U.S. 10/072,076; SEQ ID NO: 394 | 10160 |
| PN1445 | VH | PRT | U.S. 10/072,076; SEQ ID NO: 386 | 10161 |
| PN1446 | VH | PRT | U.S. 10/072,076; SEQ ID NO: 342 | 10162 |
| PN1447 | VH | PRT | U.S. 10/072,076; SEQ ID NO: 468 | 10163 |
| PN1448 | VH | PRT | U.S. 10/072,076; SEQ ID NO: 334 | 10164 |
| PN1449 | VH | PRT | U.S. 10/072,076; SEQ ID NO: 428 | 10165 |
| PN1450 | VH | PRT | U.S. 10/072,076; SEQ ID NO: 310 | 10166 |
| PN1451 | VH | PRT | U.S. 10/072,076; SEQ ID NO: 322 | 10167 |
| PN1452 | VH | PRT | U.S. 10/072,076; SEQ ID NO: 318 | 10168 |
| PN1453 | VH | PRT | U.S. 10/072,076; SEQ ID NO: 452 | 10169 |
| PN1454 | VH | PRT | U.S. 10/072,076; SEQ ID NO: 476 | 10170 |
| PN1455 | VH | PRT | U.S. 10/072,076; SEQ ID NO: 472 | 10171 |
| PN1456 | VH | PRT | U.S. 10/072,076; SEQ ID NO: 430 | 10172 |
| PN1457 | VH | PRT | U.S. 10/072,076; SEQ ID NO: 434 | 10173 |
| PN1458 | VH | PRT | U.S. 10/072,076; SEQ ID NO: 326 | 10174 |
| PN1459 | VH | PRT | U.S. 10/072,076; SEQ ID NO: 514 | 10175 |
| PN1460 | VH | PRT | U.S. 10/072,076; SEQ ID NO: 266 | 10176 |
| PN1461 | VH | PRT | U.S. 10/072,076; SEQ ID NO: 510 | 10177 |
| PN1462 | VH | PRT | U.S. 10/072,076; SEQ ID NO: 488 | 10178 |
| PN1463 | VH | PRT | U.S. 10/072,076; SEQ ID NO: 492 | 10179 |
| PN1464 | VH | PRT | U.S. 10/072,076; SEQ ID NO: 426 | 10180 |
| PN1465 | VH | PRT | U.S. 10/072,076; SEQ ID NO: 440 | 10181 |
| PN1466 | VH | PRT | U.S. 10/072,076; SEQ ID NO: 482 | 10182 |
| PN1467 | VH | PRT | U.S. 10/072,076; SEQ ID NO: 460 | 10183 |
| PN1468 | VH | PRT | U.S. 10/072,076; SEQ ID NO: 186 | 10184 |
| PN1469 | VH | PRT | U.S. 10/072,076; SEQ ID NO: 544 | 10185 |
| PN1470 | VH | PRT | U.S. 10/072,076; SEQ ID NO: 54 | 10186 |
| PN1471 | VH | PRT | U.S. 10/072,076; SEQ ID NO: 290 | 10187 |
| PN1472 | VH | PRT | U.S. 10/072,076; SEQ ID NO: 298 | 10188 |
| PN1473 | VH | PRT | U.S. 10/072,076; SEQ ID NO: 422 | 10189 |
| PN1474 | VH | PRT | U.S. 10/072,076; SEQ ID NO: 190 | 10190 |
| PN1475 | VH | PRT | U.S. 10/072,076; SEQ ID NO: 456 | 10191 |
| PN1476 | VH | PRT | U.S. 10/072,076; SEQ ID NO: 46 | 10192 |
| PN1477 | VH | PRT | U.S. 10/072,076; SEQ ID NO: 530 | 10193 |
| PN1478 | VH | PRT | U.S. 10/072,076; SEQ ID NO: 526 | 10194 |
| PN1479 | VH | PRT | U.S. 10/072,076; SEQ ID NO: 254 | 10195 |
| PN1480 | VH | PRT | U.S. 10/072,076; SEQ ID NO: 238 | 10196 |
| PN1481 | VH | PRT | U.S. 10/072,076; SEQ ID NO: 286 | 10197 |
| PN1482 | VH | PRT | U.S. 10/072,076; SEQ ID NO: 242 | 10198 |
| PN1483 | VH | PRT | U.S. 10/072,076; SEQ ID NO: 418 | 10199 |
| PN1484 | VH | PRT | U.S. 10/072,076; SEQ ID NO: 354 | 10200 |
| PN1485 | VH | PRT | U.S. 10/072,076; SEQ ID NO: 502 | 10201 |
| PN1486 | VH | PRT | U.S. 10/072,076; SEQ ID NO: 524 | 10202 |
| PN1487 | VH | PRT | U.S. 10/072,076; SEQ ID NO: 470 | 10203 |
| PN1488 | VH | PRT | U.S. 10/072,076; SEQ ID NO: 294 | 10204 |
| PN1489 | VH | PRT | U.S. 10/072,076; SEQ ID NO: 727 | 10205 |
| PN1490 | VH | PRT | US20160207996A1; SEQ ID NO: 608 | 10206 |
| PN1491 | VH | PRT | US20160207996A1; SEQ ID NO: 600 | 10207 |
| PN1492 | VH | PRT | US20160207996A1; SEQ ID NO: 607 | 10208 |
| PN1493 | VH | PRT | US20160207996A1; SEQ ID NO: 605 | 10209 |
| PN1494 | VI | DNA | U.S. 10/072,076; SEQ ID NO: 551 | 10210 |
| PN1495 | VL | DNA | US20180142038A1; SEQ ID NO: 192 | 10211 |
| PN1496 | VL | DNA | U.S. 10/202,450; SEQ ID NO: 16 | 10212 |
| PN1497 | VL | DNA | US20180142038A1; SEQ ID NO: 72 | 10213 |
| PN1498 | VL | DNA | US20180142038A1; SEQ ID NO: 112 | 10214 |
| PN1499 | VL | DNA | U.S. 10/072,076; SEQ ID NO: 798 | 10215 |
| PN1500 | VL | DNA | U.S. 10/072,076; SEQ ID NO: 143 | 10216 |
| PN1501 | VL | DNA | U.S. 10/072,076; SEQ ID NO: 199 | 10217 |
| PN1502 | VL | DNA | U.S. 10/072,076; SEQ ID NO: 203 | 10218 |
| PN1503 | VL | DNA | U.S. 10/072,076; SEQ ID NO: 279 | 10219 |
| PN1504 | VL | DNA | U.S. 10/072,076; SEQ ID NO: 649 | 10220 |
| PN1505 | VL | DNA | U.S. 10/072,076; SEQ ID NO: 609 | 10221 |
| PN1506 | VL | DNA | U.S. 10/072,076; SEQ ID NO: 627 | 10222 |
| PN1507 | VL | DNA | U.S. 10/072,076; SEQ ID NO: 343 | 10223 |
| PN1508 | VL | DNA | U.S. 10/072,076; SEQ ID NO: 371 | 10224 |
| PN1509 | VL | DNA | U.S. 10/072,076; SEQ ID NO: 399 | 10225 |
| PN1510 | VL | DNA | U.S. 10/072,076; SEQ ID NO: 255 | 10226 |
| PN1511 | VL | DNA | U.S. 10/072,076; SEQ ID NO: 597 | 10227 |
| PN1512 | VL | DNA | U.S. 10/072,076; SEQ ID NO: 625 | 10228 |
| PN1513 | VL | DNA | U.S. 10/072,076; SEQ ID NO: 605 | 10229 |
| PN1514 | VL | DNA | U.S. 10/072,076; SEQ ID NO: 613 | 10230 |
| PN1515 | VL | DNA | U.S. 10/072,076; SEQ ID NO: 611 | 10231 |
| PN1516 | VL | DNA | U.S. 10/072,076; SEQ ID NO: 247 | 10232 |
| PN1517 | VL | DNA | U.S. 10/072,076; SEQ ID NO: 39 | 10233 |
| PN1518 | VL | DNA | U.S. 10/072,076; SEQ ID NO: 599 | 10234 |
| PN1519 | VL | DNA | U.S. 10/072,076; SEQ ID NO: 621 | 10235 |
| PN1520 | VL | DNA | U.S. 10/072,076; SEQ ID NO: 307 | 10236 |
| PN1521 | VL | DNA | U.S. 10/072,076; SEQ ID NO: 593 | 10237 |
| PN1522 | VL | DNA | U.S. 10/072,076; SEQ ID NO: 31 | 10238 |
| PN1523 | VL | DNA | U.S. 10/072,076; SEQ ID NO: 295 | 10239 |
| PN1524 | VL | DNA | U.S. 10/072,076; SEQ ID NO: 55 | 10240 |
| PN1525 | VL | DNA | U.S. 10/072,076; SEQ ID NO: 311 | 10241 |
| PN1526 | VL | DNA | U.S. 10/072,076; SEQ ID NO: 643 | 10242 |
| PN1527 | VL | DNA | U.S. 10/072,076; SEQ ID NO: 631 | 10243 |
| PN1528 | VL | DNA | U.S. 10/072,076; SEQ ID NO: 710 | 10244 |
| PN1529 | VL | DNA | U.S. 10/072,076; SEQ ID NO: 619 | 10245 |
| PN1530 | VL | DNA | U.S. 10/072,076; SEQ ID NO: 379 | 10246 |
| PN1531 | VL | DNA | U.S. 10/072,076; SEQ ID NO: 47 | 10247 |
| PN1532 | VL | DNA | U.S. 10/072,076; SEQ ID NO: 615 | 10248 |
| PN1533 | VL | DNA | U.S. 10/072,076; SEQ ID NO: 43 | 10249 |
| PN1534 | VL | DNA | U.S. 10/072,076; SEQ ID NO: 271 | 10250 |
| PN1535 | VL | DNA | U.S. 10/072,076; SEQ ID NO: 663 | 10251 |
| PN1536 | VL | DNA | U.S. 10/072,076; SEQ ID NO: 665 | 10252 |
| PN1537 | VL | DNA | U.S. 10/072,076; SEQ ID NO: 601 | 10253 |
| PN1538 | VL | DNA | U.S. 10/072,076; SEQ ID NO: 603 | 10254 |
| PN1539 | VL | DNA | U.S. 10/072,076; SEQ ID NO: 259 | 10255 |
| PN1540 | VL | DNA | U.S. 10/072,076; SEQ ID NO: 319 | 10256 |
| PN1541 | VL | DNA | U.S. 10/072,076; SEQ ID NO: 367 | 10257 |
| PN1542 | VL | DNA | U.S. 10/072,076; SEQ ID NO: 335 | 10258 |
| PN1543 | VL | DNA | U.S. 10/072,076; SEQ ID NO: 415 | 10259 |
| PN1544 | VL | DNA | U.S. 10/072,076; SEQ ID NO: 419 | 10260 |
| PN1545 | VL | DNA | U.S. 10/072,076; SEQ ID NO: 355 | 10261 |
| PN1546 | VL | DNA | U.S. 10/072,076; SEQ ID NO: 659 | 10262 |
| PN1547 | VL | DNA | U.S. 10/072,076; SEQ ID NO: 403 | 10263 |
| PN1548 | VL | DNA | U.S. 10/072,076; SEQ ID NO: 91 | 10264 |
| PN1549 | VL | DNA | U.S. 10/072,076; SEQ ID NO: 95 | 10265 |
| PN1550 | VL | DNA | U.S. 10/072,076; SEQ ID NO: 359 | 10266 |
| PN1551 | VL | DNA | U.S. 10/072,076; SEQ ID NO: 347 | 10267 |
| PN1552 | VL | DNA | U.S. 10/072,076; SEQ ID NO: 363 | 10268 |
| PN1553 | VL | DNA | U.S. 10/072,076; SEQ ID NO: 383 | 10269 |
| PN1554 | VL | DNA | U.S. 10/072,076; SEQ ID NO: 411 | 10270 |
| PN1555 | VL | DNA | U.S. 10/072,076; SEQ ID NO: 267 | 10271 |
| PN1556 | VL | DNA | U.S. 10/072,076; SEQ ID NO: 275 | 10272 |
| PN1557 | VL | DNA | U.S. 10/072,076; SEQ ID NO: 35 | 10273 |
| PN1558 | VL | DNA | U.S. 10/072,076; SEQ ID NO: 395 | 10274 |
| PN1559 | VL | DNA | U.S. 10/072,076; SEQ ID NO: 183 | 10275 |
| PN1560 | VL | DNA | U.S. 10/072,076; SEQ ID NO: 637 | 10276 |
| PN1561 | VL | DNA | U.S. 10/072,076; SEQ ID NO: 573 | 10277 |
| PN1562 | VL | DNA | U.S. 10/072,076; SEQ ID NO: 159 | 10278 |
| PN1563 | VL | DNA | U.S. 10/072,076; SEQ ID NO: 563 | 10279 |
| PN1564 | VL | DNA | U.S. 10/072,076; SEQ ID NO: 549 | 10280 |
| PN1565 | VL | DNA | U.S. 10/072,076; SEQ ID NO: 115 | 10281 |
| PN1566 | VL | DNA | U.S. 10/072,076; SEQ ID NO: 571 | 10282 |
| PN1567 | VL | DNA | U.S. 10/072,076; SEQ ID NO: 591 | 10283 |
| PN1568 | VL | DNA | U.S. 10/072,076; SEQ ID NO: 315 | 10284 |
| PN1569 | VL | DNA | U.S. 10/072,076; SEQ ID NO: 653 | 10285 |
| PN1570 | VL | DNA | U.S. 10/072,076; SEQ ID NO: 67 | 10286 |
| PN1571 | VL | DNA | U.S. 10/072,076; SEQ ID NO: 71 | 10287 |
| PN1572 | VL | DNA | U.S. 10/072,076; SEQ ID NO: 645 | 10288 |
| PN1573 | VL | DNA | U.S. 10/072,076; SEQ ID NO: 865 | 10289 |
| PN1574 | VL | DNA | U.S. 10/072,076; SEQ ID NO: 83 | 10290 |
| PN1575 | VL | DNA | U.S. 10/072,076; SEQ ID NO: 407 | 10291 |
| PN1576 | VL | DNA | U.S. 10/072,076; SEQ ID NO: 565 | 10292 |
| PN1577 | VL | DNA | U.S. 10/072,076; SEQ ID NO: 167 | 10293 |
| PN1578 | VL | DNA | U.S. 10/072,076; SEQ ID NO: 567 | 10294 |
| PN1579 | VL | DNA | U.S. 10/072,076; SEQ ID NO: 873 | 10295 |
| PN1580 | VL | DNA | U.S. 10/072,076; SEQ ID NO: 59 | 10296 |
| PN1581 | VL | DNA | U.S. 10/072,076; SEQ ID NO: 581 | 10297 |
| PN1582 | VL | DNA | U.S. 10/072,076; SEQ ID NO: 555 | 10298 |
| PN1583 | VL | DNA | U.S. 10/072,076; SEQ ID NO: 607 | 10299 |
| PN1584 | VL | DNA | U.S. 10/072,076; SEQ ID NO: 577 | 10300 |
| PN1585 | VL | DNA | U.S. 10/072,076; SEQ ID NO: 287 | 10301 |
| PN1586 | VL | DNA | U.S. 10/072,076; SEQ ID NO: 323 | 10302 |
| PN1587 | VL | DNA | U.S. 10/072,076; SEQ ID NO: 583 | 10303 |
| PN1588 | VL | DNA | U.S. 10/072,076; SEQ ID NO: 557 | 10304 |
| PN1589 | VL | DNA | U.S. 10/072,076; SEQ ID NO: 163 | 10305 |
| PN1590 | VL | DNA | U.S. 10/072,076; SEQ ID NO: 569 | 10306 |
| PN1591 | VL | DNA | U.S. 10/072,076; SEQ ID NO: 211 | 10307 |
| PN1592 | VL | DNA | U.S. 10/072,076; SEQ ID NO: 351 | 10308 |
| PN1593 | VL | DNA | U.S. 10/072,076; SEQ ID NO: 235 | 10309 |
| PN1594 | VL | DNA | U.S. 10/072,076; SEQ ID NO: 327 | 10310 |
| PN1595 | VL | DNA | U.S. 10/072,076; SEQ ID NO: 63 | 10311 |
| PN1596 | VL | DNA | U.S. 10/072,076; SEQ ID NO: 75 | 10312 |
| PN1597 | VL | DNA | U.S. 10/072,076; SEQ ID NO: 661 | 10313 |
| PN1598 | VL | DNA | U.S. 10/072,076; SEQ ID NO: 547 | 10314 |
| PN1599 | VL | DNA | U.S. 10/072,076; SEQ ID NO: 151 | 10315 |
| PN1600 | VL | DNA | U.S. 10/072,076; SEQ ID NO: 633 | 10316 |
| PN1601 | VL | DNA | U.S. 10/072,076; SEQ ID NO: 617 | 10317 |
| PN1602 | VL | DNA | U.S. 10/072,076; SEQ ID NO: 623 | 10318 |
| PN1603 | VL | DNA | U.S. 10/072,076; SEQ ID NO: 103 | 10319 |
| PN1604 | VL | DNA | U.S. 10/072,076; SEQ ID NO: 107 | 10320 |
| PN1605 | VL | DNA | U.S. 10/072,076; SEQ ID NO: 706 | 10321 |
| PN1606 | VL | DNA | U.S. 10/072,076; SEQ ID NO: 283 | 10322 |
| PN1607 | VL | DNA | U.S. 10/072,076; SEQ ID NO: 263 | 10323 |
| PN1608 | VL | DNA | U.S. 10/072,076; SEQ ID NO: 299 | 10324 |
| PN1609 | VL | DNA | U.S. 10/072,076; SEQ ID NO: 191 | 10325 |
| PN1610 | VL | DNA | U.S. 10/072,076; SEQ ID NO: 131 | 10326 |
| PN1611 | VL | DNA | US20160207996A1; SEQ ID NO: 306 | 10327 |
| PN1612 | VL | DNA | U.S. 10/072,076; SEQ ID NO: 553 | 10328 |
| PN1613 | VL | DNA | U.S. 10/072,076; SEQ ID NO: 223 | 10329 |
| PN1614 | VL | DNA | U.S. 10/072,076; SEQ ID NO: 375 | 10330 |
| PN1615 | VL | DNA | U.S. 10/072,076; SEQ ID NO: 639 | 10331 |
| PN1616 | VL | DNA | U.S. 10/072,076; SEQ ID NO: 647 | 10332 |
| PN1617 | VL | DNA | U.S. 10/072,076; SEQ ID NO: 239 | 10333 |
| PN1618 | VL | DNA | U.S. 10/072,076; SEQ ID NO: 339 | 10334 |
| PN1619 | VL | DNA | U.S. 10/072,076; SEQ ID NO: 219 | 10335 |
| PN1620 | VL | DNA | U.S. 10/072,076; SEQ ID NO: 561 | 10336 |
| PN1621 | VL | DNA | U.S. 10/072,076; SEQ ID NO: 123 | 10337 |
| PN1622 | VL | DNA | U.S. 10/072,076; SEQ ID NO: 171 | 10338 |
| PN1623 | VL | DNA | U.S. 10/072,076; SEQ ID NO: 139 | 10339 |
| PN1624 | VL | DNA | U.S. 10/072,076; SEQ ID NO: 559 | 10340 |
| PN1625 | VL | DNA | U.S. 10/072,076; SEQ ID NO: 3 | 10341 |
| PN1626 | VL | DNA | U.S. 10/072,076; SEQ ID NO: 187 | 10342 |
| PN1627 | VL | DNA | U.S. 10/072,076; SEQ ID NO: 127 | 10343 |
| PN1628 | VL | DNA | U.S. 10/072,076; SEQ ID NO: 589 | 10344 |
| PN1629 | VL | DNA | U.S. 10/072,076; SEQ ID NO: 119 | 10345 |
| PN1630 | VL | DNA | U.S. 10/072,076; SEQ ID NO: 179 | 10346 |
| PN1631 | VL | DNA | U.S. 10/072,076; SEQ ID NO: 579 | 10347 |
| PN1632 | VL | DNA | U.S. 10/072,076; SEQ ID NO: 147 | 10348 |
| PN1633 | VL | DNA | U.S. 10/072,076; SEQ ID NO: 195 | 10349 |
| PN1634 | VL | DNA | U.S. 10/072,076; SEQ ID NO: 575 | 10350 |
| PN1635 | VL | DNA | U.S. 10/072,076; SEQ ID NO: 331 | 10351 |
| PN1636 | VL | DNA | U.S. 10/072,076; SEQ ID NO: 79 | 10352 |
| PN1637 | VL | DNA | U.S. 10/072,076; SEQ ID NO: 175 | 10353 |
| PN1638 | VL | DNA | U.S. 10/072,076; SEQ ID NO: 861 | 10354 |
| PN1639 | VL | DNA | U.S. 10/072,076; SEQ ID NO: 869 | 10355 |
| PN1640 | VL | DNA | U.S. 10/072,076; SEQ ID NO: 694 | 10356 |
| PN1641 | VL | DNA | U.S. 10/072,076; SEQ ID NO: 655 | 10357 |
| PN1642 | VL | DNA | U.S. 10/072,076; SEQ ID NO: 51 | 10358 |
| PN1643 | VL | DNA | U.S. 10/072,076; SEQ ID NO: 667 | 10359 |
| PN1644 | VL | DNA | US20180142038A1; SEQ ID NO: 152 | 10360 |
| PN1645 | VL | DNA | US20180142038A1; SEQ ID NO: 32 | 10361 |
| PN1646 | VL | DNA | U.S. 10/072,076; SEQ ID NO: 215 | 10362 |
| PN1647 | VL | DNA | U.S. 10/072,076; SEQ ID NO: 7 | 10363 |
| PN1648 | VL | DNA | U.S. 10/072,076; SEQ ID NO: 587 | 10364 |
| PN1649 | VL | DNA | U.S. 10/072,076; SEQ ID NO: 11 | 10365 |
| PN1650 | VL | DNA | U.S. 10/072,076; SEQ ID NO: 15 | 10366 |
| PN1651 | VL | DNA | U.S. 10/072,076; SEQ ID NO: 585 | 10367 |
| PN1652 | VL | DNA | U.S. 10/072,076; SEQ ID NO: 734 | 10368 |
| PN1653 | VL | DNA | U.S. 10/072,076; SEQ ID NO: 766 | 10369 |
| PN1654 | VL | DNA | U.S. 10/072,076; SEQ ID NO: 750 | 10370 |
| PN1655 | VL | DNA | U.S. 10/072,076; SEQ ID NO: 227 | 10371 |
| PN1656 | VL | DNA | U.S. 10/072,076; SEQ ID NO: 27 | 10372 |
| PN1657 | VL | DNA | U.S. 10/072,076; SEQ ID NO: 231 | 10373 |
| PN1658 | VL | DNA | U.S. 10/072,076; SEQ ID NO: 641 | 10374 |
| PN1659 | VL | DNA | U.S. 10/072,076; SEQ ID NO: 243 | 10375 |
| PN1660 | VL | DNA | U.S. 10/072,076; SEQ ID NO: 291 | 10376 |
| PN1661 | VL | DNA | U.S. 10/072,076; SEQ ID NO: 423 | 10377 |
| PN1662 | VL | DNA | U.S. 10/072,076; SEQ ID NO: 19 | 10378 |
| PN1663 | VL | DNA | U.S. 10/072,076; SEQ ID NO: 207 | 10379 |
| PN1664 | VL | DNA | U.S. 10/072,076; SEQ ID NO: 135 | 10380 |
| PN1665 | VL | DNA | U.S. 10/072,076; SEQ ID NO: 87 | 10381 |
| PN1666 | VL | DNA | U.S. 10/072,076; SEQ ID NO: 635 | 10382 |
| PN1667 | VL | DNA | U.S. 10/072,076; SEQ ID NO: 657 | 10383 |
| PN1668 | VL | DNA | U.S. 10/072,076; SEQ ID NO: 155 | 10384 |
| PN1669 | VL | DNA | U.S. 10/072,076; SEQ ID NO: 99 | 10385 |
| PN1670 | VL | DNA | U.S. 10/072,076; SEQ ID NO: 111 | 10386 |
| PN1671 | VL | DNA | U.S. 10/072,076; SEQ ID NO: 595 | 10387 |
| PN1672 | VL | DNA | U.S. 10/072,076; SEQ ID NO: 303 | 10388 |
| PN1673 | VL | DNA | U.S. 10/072,076; SEQ ID NO: 251 | 10389 |
| PN1674 | VL | DNA | U.S. 10/072,076; SEQ ID NO: 629 | 10390 |
| PN1675 | VL | DNA | US20160207996A1; SEQ ID NO: 308 | 10391 |
| PN1676 | VL | DNA | US20160207996A1; SEQ ID NO: 304 | 10392 |
| PN1677 | VL | PRT | U.S. 10/072,076; SEQ ID NO: 232 | 10393 |
| PN1678 | VL | PRT | U.S. 10/072,076; SEQ ID NO: 100 | 10394 |
| PN1679 | VL | PRT | U.S. 10/072,076; SEQ ID NO: 112 | 10395 |
| PN1680 | VL | PRT | U.S. 10/072,076; SEQ ID NO: 244 | 10396 |
| PN1681 | VL | PRT | U.S. 10/072,076; SEQ ID NO: 292 | 10397 |
| PN1682 | VL | PRT | U.S. 10/072,076; SEQ ID NO: 424 | 10398 |
| PN1683 | VL | PRT | U.S. 10/072,076; SEQ ID NO: 642 | 10399 |
| PN1684 | VL | PRT | U.S. 10/072,076; SEQ ID NO: 658 | 10400 |
| PN1685 | VL | PRT | U.S. 10/072,076; SEQ ID NO: 88 | 10401 |
| PN1686 | VL | PRT | U.S. 10/072,076; SEQ ID NO: 636 | 10402 |
| PN1687 | VL | PRT | U.S. 10/072,076; SEQ ID NO: 136 | 10403 |
| PN1688 | VL | PRT | U.S. 10/072,076; SEQ ID NO: 156 | 10404 |
| PN1689 | VL | PRT | U.S. 10/072,076; SEQ ID NO: 208 | 10405 |
| PN1690 | VL | PRT | U.S. 10/072,076; SEQ ID NO: 20 | 10406 |
| PN1691 | VL | PRT | U.S. 10/072,076; SEQ ID NO: 596 | 10407 |
| PN1692 | VL | PRT | U.S. 10/072,076; SEQ ID NO: 252 | 10408 |
| PN1693 | VL | PRT | U.S. 10/072,076; SEQ ID NO: 304 | 10409 |
| PN1694 | VL | PRT | U.S. 10/072,076; SEQ ID NO: 630 | 10410 |
| PN1695 | VL | PRT | US20160207996A1; SEQ ID NO: 255 | 10411 |
| PN1696 | VL | PRT | US20160207996A1; SEQ ID NO: 256 | 10412 |
| PN1697 | VL | PRT | US20160207996A1; SEQ ID NO: 303 | 10413 |
| PN1698 | VL | PRT | US20160207996A1; SEQ ID NO: 252 | 10414 |
| PN1699 | VL | PRT | US20160207996A1; SEQ ID NO: 253 | 10415 |
| PN1700 | VL | PRT | US20160207996A1; SEQ ID NO: 257 | 10416 |
| PN1701 | VL | PRT | US20160207996A1; SEQ ID NO: 258 | 10417 |
| PN1702 | VL | PRT | US20160207996A1; SEQ ID NO: 259 | 10418 |
| PN1703 | VL | PRT | U.S. 10/072,076; SEQ ID NO: 204 | 10419 |
| PN1704 | VL | PRT | U.S. 10/072,076; SEQ ID NO: 200 | 10420 |
| PN1705 | VL | PRT | U.S. 10/072,076; SEQ ID NO: 196 | 10421 |
| PN1706 | VL | PRT | U.S. 10/072,076; SEQ ID NO: 144 | 10422 |
| PN1707 | VL | PRT | U.S. 10/072,076; SEQ ID NO: 576 | 10423 |
| PN1708 | VL | PRT | U.S. 10/072,076; SEQ ID NO: 148 | 10424 |
| PN1709 | VL | PRT | U.S. 10/072,076; SEQ ID NO: 340 | 10425 |
| PN1710 | VL | PRT | U.S. 10/072,076; SEQ ID NO: 648 | 10426 |
| PN1711 | VL | PRT | U.S. 10/072,076; SEQ ID NO: 240 | 10427 |
| PN1712 | VL | PRT | U.S. 10/072,076; SEQ ID NO: 640 | 10428 |
| PN1713 | VL | PRT | U.S. 10/072,076; SEQ ID NO: 376 | 10429 |
| PN1714 | VL | PRT | U.S. 10/072,076; SEQ ID NO: 188 | 10430 |
| PN1715 | VL | PRT | U.S. 10/072,076; SEQ ID NO: 128 | 10431 |
| PN1716 | VL | PRT | U.S. 10/072,076; SEQ ID NO: 590 | 10432 |
| PN1717 | VL | PRT | U.S. 10/072,076; SEQ ID NO: 4 | 10433 |
| PN1718 | VL | PRT | U.S. 10/072,076; SEQ ID NO: 560 | 10434 |
| PN1719 | VL | PRT | U.S. 10/072,076; SEQ ID NO: 140 | 10435 |
| PN1720 | VL | PRT | U.S. 10/072,076; SEQ ID NO: 124 | 10436 |
| PN1721 | VL | PRT | U.S. 10/072,076; SEQ ID NO: 562 | 10437 |
| PN1722 | VL | PRT | U.S. 10/072,076; SEQ ID NO: 220 | 10438 |
| PN1723 | VL | PRT | U.S. 10/072,076; SEQ ID NO: 172 | 10439 |
| PN1724 | VL | PRT | U.S. 10/072,076; SEQ ID NO: 580 | 10440 |
| PN1725 | VL | PRT | U.S. 10/072,076; SEQ ID NO: 120 | 10441 |
| PN1726 | VL | PRT | U.S. 10/072,076; SEQ ID NO: 180 | 10442 |
| PN1727 | VL | PRT | U.S. 10/072,076; SEQ ID NO: 260 | 10443 |
| PN1728 | VL | PRT | U.S. 10/072,076; SEQ ID NO: 604 | 10444 |
| PN1729 | VL | PRT | U.S. 10/072,076; SEQ ID NO: 626 | 10445 |
| PN1730 | VL | PRT | U.S. 10/072,076; SEQ ID NO: 598 | 10446 |
| PN1731 | VL | PRT | U.S. 10/072,076; SEQ ID NO: 606 | 10447 |
| PN1732 | VL | PRT | U.S. 10/072,076; SEQ ID NO: 272 | 10448 |
| PN1733 | VL | PRT | U.S. 10/072,076; SEQ ID NO: 44 | 10449 |
| PN1734 | VL | PRT | U.S. 10/072,076; SEQ ID NO: 48 | 10450 |
| PN1735 | VL | PRT | U.S. 10/072,076; SEQ ID NO: 616 | 10451 |
| PN1736 | VL | PRT | U.S. 10/072,076; SEQ ID NO: 614 | 10452 |
| PN1737 | VL | PRT | U.S. 10/072,076; SEQ ID NO: 612 | 10453 |
| PN1738 | VL | PRT | U.S. 10/072,076; SEQ ID NO: 632 | 10454 |
| PN1739 | VL | PRT | U.S. 10/072,076; SEQ ID NO: 594 | 10455 |
| PN1740 | VL | PRT | U.S. 10/072,076; SEQ ID NO: 380 | 10456 |
| PN1741 | VL | PRT | U.S. 10/072,076; SEQ ID NO: 308 | 10457 |
| PN1742 | VL | PRT | U.S. 10/072,076; SEQ ID NO: 711 | 10458 |
| PN1743 | VL | PRT | U.S. 10/072,076; SEQ ID NO: 32 | 10459 |
| PN1744 | VL | PRT | U.S. 10/072,076; SEQ ID NO: 622 | 10460 |
| PN1745 | VL | PRT | U.S. 10/072,076; SEQ ID NO: 600 | 10461 |
| PN1746 | VL | PRT | U.S. 10/072,076; SEQ ID NO: 248 | 10462 |
| PN1747 | VL | PRT | U.S. 10/072,076; SEQ ID NO: 296 | 10463 |
| PN1748 | VL | PRT | U.S. 10/072,076; SEQ ID NO: 312 | 10464 |
| PN1749 | VL | PRT | U.S. 10/072,076; SEQ ID NO: 644 | 10465 |
| PN1750 | VL | PRT | U.S. 10/072,076; SEQ ID NO: 620 | 10466 |
| PN1751 | VL | PRT | U.S. 10/072,076; SEQ ID NO: 264 | 10467 |
| PN1752 | VL | PRT | U.S. 10/072,076; SEQ ID NO: 300 | 10468 |
| PN1753 | VL | PRT | U.S. 10/072,076; SEQ ID NO: 602 | 10469 |
| PN1754 | VL | PRT | U.S. 10/072,076; SEQ ID NO: 664 | 10470 |
| PN1755 | VL | PRT | U.S. 10/072,076; SEQ ID NO: 256 | 10471 |
| PN1756 | VL | PRT | U.S. 10/072,076; SEQ ID NO: 320 | 10472 |
| PN1757 | VL | PRT | U.S. 10/072,076; SEQ ID NO: 628 | 10473 |
| PN1758 | VL | PRT | U.S. 10/072,076; SEQ ID NO: 344 | 10474 |
| PN1759 | VL | PRT | U.S. 10/072,076; SEQ ID NO: 610 | 10475 |
| PN1760 | VL | PRT | U.S. 10/072,076; SEQ ID NO: 650 | 10476 |
| PN1761 | VL | PRT | U.S. 10/072,076; SEQ ID NO: 280 | 10477 |
| PN1762 | VL | PRT | U.S. 10/072,076; SEQ ID NO: 662 | 10478 |
| PN1763 | VL | PRT | U.S. 10/072,076; SEQ ID NO: 328 | 10479 |
| PN1764 | VL | PRT | U.S. 10/072,076; SEQ ID NO: 76 | 10480 |
| PN1765 | VL | PRT | U.S. 10/072,076; SEQ ID NO: 236 | 10481 |
| PN1766 | VL | PRT | U.S. 10/072,076; SEQ ID NO: 352 | 10482 |
| PN1767 | VL | PRT | U.S. 10/072,076; SEQ ID NO: 212 | 10483 |
| PN1768 | VL | PRT | U.S. 10/072,076; SEQ ID NO: 184 | 10484 |
| PN1769 | VL | PRT | US20160207996A1; SEQ ID NO: 250 | 10485 |
| PN1770 | VL | PRT | U.S. 10/072,076; SEQ ID NO: 224 | 10486 |
| PN1771 | VL | PRT | U.S. 10/072,076; SEQ ID NO: 72 | 10487 |
| PN1772 | VL | PRT | U.S. 10/072,076; SEQ ID NO: 68 | 10488 |
| PN1773 | VL | PRT | U.S. 10/072,076; SEQ ID NO: 654 | 10489 |
| PN1774 | VL | PRT | U.S. 10/072,076; SEQ ID NO: 316 | 10490 |
| PN1775 | VL | PRT | U.S. 10/072,076; SEQ ID NO: 646 | 10491 |
| PN1776 | VL | PRT | U.S. 10/072,076; SEQ ID NO: 84 | 10492 |
| PN1777 | VL | PRT | U.S. 10/072,076; SEQ ID NO: 368 | 10493 |
| PN1778 | VL | PRT | U.S. 10/072,076; SEQ ID NO: 164 | 10494 |
| PN1779 | VL | PRT | U.S. 10/072,076; SEQ ID NO: 356 | 10495 |
| PN1780 | VL | PRT | U.S. 10/072,076; SEQ ID NO: 420 | 10496 |
| PN1781 | VL | PRT | U.S. 10/072,076; SEQ ID NO: 416 | 10497 |
| PN1782 | VL | PRT | U.S. 10/072,076; SEQ ID NO: 336 | 10498 |
| PN1783 | VL | PRT | U.S. 10/072,076; SEQ ID NO: 408 | 10499 |
| PN1784 | VL | PRT | U.S. 10/072,076; SEQ ID NO: 566 | 10500 |
| PN1785 | VL | PRT | U.S. 10/072,076; SEQ ID NO: 556 | 10501 |
| PN1786 | VL | PRT | U.S. 10/072,076; SEQ ID NO: 582 | 10502 |
| PN1787 | VL | PRT | U.S. 10/072,076; SEQ ID NO: 168 | 10503 |
| PN1788 | VL | PRT | U.S. 10/072,076; SEQ ID NO: 568 | 10504 |
| PN1789 | VL | PRT | U.S. 10/072,076; SEQ ID NO: 874 | 10505 |
| PN1790 | VL | PRT | U.S. 10/072,076; SEQ ID NO: 60 | 10506 |
| PN1791 | VL | PRT | U.S. 10/072,076; SEQ ID NO: 584 | 10507 |
| PN1792 | VL | PRT | U.S. 10/072,076; SEQ ID NO: 360 | 10508 |
| PN1793 | VL | PRT | U.S. 10/072,076; SEQ ID NO: 412 | 10509 |
| PN1794 | VL | PRT | U.S. 10/072,076; SEQ ID NO: 384 | 10510 |
| PN1795 | VL | PRT | U.S. 10/072,076; SEQ ID NO: 348 | 10511 |
| PN1796 | VL | PRT | U.S. 10/072,076; SEQ ID NO: 96 | 10512 |
| PN1797 | VL | PRT | U.S. 10/072,076; SEQ ID NO: 92 | 10513 |
| PN1798 | VL | PRT | U.S. 10/072,076; SEQ ID NO: 404 | 10514 |
| PN1799 | VL | PRT | U.S. 10/072,076; SEQ ID NO: 268 | 10515 |
| PN1800 | VL | PRT | U.S. 10/072,076; SEQ ID NO: 660 | 10516 |
| PN1801 | VL | PRT | U.S. 10/072,076; SEQ ID NO: 578 | 10517 |
| PN1802 | VL | PRT | U.S. 10/072,076; SEQ ID NO: 288 | 10518 |
| PN1803 | VL | PRT | U.S. 10/072,076; SEQ ID NO: 324 | 10519 |
| PN1804 | VL | PRT | U.S. 10/072,076; SEQ ID NO: 558 | 10520 |
| PN1805 | VL | PRT | U.S. 10/072,076; SEQ ID NO: 608 | 10521 |
| PN1806 | VL | PRT | U.S. 10/072,076; SEQ ID NO: 570 | 10522 |
| PN1807 | VL | PRT | U.S. 10/072,076; SEQ ID NO: 574 | 10523 |
| PN1808 | VL | PRT | U.S. 10/072,076; SEQ ID NO: 564 | 10524 |
| PN1809 | VL | PRT | U.S. 10/072,076; SEQ ID NO: 160 | 10525 |
| PN1810 | VL | PRT | U.S. 10/072,076; SEQ ID NO: 548 | 10526 |
| PN1811 | VL | PRT | U.S. 10/072,076; SEQ ID NO: 116 | 10527 |
| PN1812 | VL | PRT | U.S. 10/072,076; SEQ ID NO: 572 | 10528 |
| PN1813 | VL | PRT | U.S. 10/072,076; SEQ ID NO: 550 | 10529 |
| PN1814 | VL | PRT | U.S. 10/072,076; SEQ ID NO: 554 | 10530 |
| PN1815 | VL | PRT | U.S. 10/072,076; SEQ ID NO: 592 | 10531 |
| PN1816 | VL | PRT | U.S. 10/072,076; SEQ ID NO: 638 | 10532 |
| PN1817 | VL | PRT | U.S. 10/072,076; SEQ ID NO: 284 | 10533 |
| PN1818 | VL | PRT | U.S. 10/072,076; SEQ ID NO: 618 | 10534 |
| PN1819 | VL | PRT | U.S. 10/072,076; SEQ ID NO: 624 | 10535 |
| PN1820 | VL | PRT | U.S. 10/072,076; SEQ ID NO: 396 | 10536 |
| PN1821 | VL | PRT | U.S. 10/072,076; SEQ ID NO: 634 | 10537 |
| PN1822 | VL | PRT | U.S. 10/072,076; SEQ ID NO: 108 | 10538 |
| PN1823 | VL | PRT | U.S. 10/072,076; SEQ ID NO: 707 | 10539 |
| PN1824 | VL | PRT | U.S. 10/072,076; SEQ ID NO: 104 | 10540 |
| PN1825 | VL | PRT | U.S. 10/072,076; SEQ ID NO: 152 | 10541 |
| PN1826 | VL | PRT | US20160207996A1; SEQ ID NO: 235 | 10542 |
| PN1827 | VL | PRT | U.S. 10/072,076; SEQ ID NO: 192 | 10543 |
| PN1828 | VL | PRT | U.S. 10/072,076; SEQ ID NO: 132 | 10544 |
| PN1829 | VL | PRT | U.S. 10/072,076; SEQ ID NO: 332 | 10545 |
| PN1830 | VL | PRT | U.S. 10/072,076; SEQ ID NO: 80 | 10546 |
| PN1831 | VL | PRT | U.S. 10/072,076; SEQ ID NO: 862 | 10547 |
| PN1832 | VL | PRT | U.S. 10/072,076; SEQ ID NO: 52 | 10548 |
| PN1833 | VL | PRT | U.S. 10/072,076; SEQ ID NO: 656 | 10549 |
| PN1834 | VL | PRT | U.S. 10/072,076; SEQ ID NO: 870 | 10550 |
| PN1835 | VL | PRT | U.S. 10/072,076; SEQ ID NO: 695 | 10551 |
| PN1836 | VL | PRT | U.S. 10/072,076; SEQ ID NO: 28 | 10552 |
| PN1837 | VL | PRT | U.S. Pat. No. 8,986,694; SEQ ID NO: 70 | 10553 |
| PN1838 | VL | PRT | US20160207996A1; SEQ ID NO: 228 | 10554 |
| PN1839 | VL | PRT | US20160207996A1; SEQ ID NO: 229 | 10555 |
| PN1840 | VL | PRT | US20160207996A1; SEQ ID NO: 227 | 10556 |
| PN1841 | VL | PRT | U.S. 10/072,076; SEQ ID NO: 668 | 10557 |
| PN1842 | VL | PRT | U.S. 10/072,076; SEQ ID NO: 228 | 10558 |
| PN1843 | VL | PRT | U.S. 10/072,076; SEQ ID NO: 751 | 10559 |
| PN1844 | VL | PRT | U.S. 10/072,076; SEQ ID NO: 588 | 10560 |
| PN1845 | VL | PRT | U.S. 10/072,076; SEQ ID NO: 767 | 10561 |
| PN1846 | VL | PRT | U.S. 10/072,076; SEQ ID NO: 176 | 10562 |
| PN1847 | VL | PRT | U.S. 10/072,076; SEQ ID NO: 16 | 10563 |
| PN1848 | VL | PRT | U.S. 10/072,076; SEQ ID NO: 12 | 10564 |
| PN1849 | VL | PRT | U.S. 10/072,076; SEQ ID NO: 586 | 10565 |
| PN1850 | VL | PRT | U.S. 10/072,076; SEQ ID NO: 735 | 10566 |
| PN1851 | VL | PRT | U.S. 10/072,076; SEQ ID NO: 8 | 10567 |
| PN1852 | VL | PRT | US20180142038A1; SEQ ID NO: 22 | 10568 |
| PN1853 | VL | PRT | US20180142038A1; SEQ ID NO: 142 | 10569 |
| PN1854 | VL | PRT | U.S. 10/072,076; SEQ ID NO: 216 | 10570 |
| PN1855 | VL | PRT | U.S. 10/072,076; SEQ ID NO: 799 | 10571 |
| PN1856 | VL | PRT | US20160207996A1; SEQ ID NO: 231 | 10572 |
| PN1857 | VL | PRT | US20180142038A1; SEQ ID NO: 182 | 10573 |
| PN1858 | VL | PRT | U.S. 10/202,450; SEQ ID NO: 8 | 10574 |
| PN1859 | VL | PRT | US20160207996A1; SEQ ID NO: 230 | 10575 |
| PN1860 | VL | PRT | U.S. 10/072,076; SEQ ID NO: 552 | 10576 |
| PN1861 | VL | PRT | US20160207996A1; SEQ ID NO: 233 | 10577 |
| PN1862 | VL | PRT | US20160207996A1; SEQ ID NO: 254 | 10578 |
| PN1863 | VL | PRT | US20160207996A1; SEQ ID NO: 251 | 10579 |
| PN1864 | VL | PRT | US20180142038A1; SEQ ID NO: 62 | 10580 |
| PN1865 | VL | PRT | US20180142038A1; SEQ ID NO: 102 | 10581 |
| PN1866 | VL | PRT | US20160207996A1; SEQ ID NO: 234 | 10582 |
| PN1867 | VL | PRT | US20160207996A1; SEQ ID NO: 232 | 10583 |
| PN1868 | VL | PRT | US20160207996A1; SEQ ID NO: 249 | 10584 |
| PN1869 | VH | PRT | WO2017139290; SEQ ID NO: 1 | 10585 |
| PN1870 | VL | PRT | WO2017139290; SEQ ID NO: 2 | 10586 |

In some embodiments, the payload region of the AAV particle comprises a nucleic acid sequence encoding a polypeptide which is an antibody, an antibody-based composition, or a fragment thereof. As anon-limiting example, the antibody may be one or more of the polypeptides listed in Table 13, or variants or fragments thereof. As another non-limiting example, the antibody may be one or more of the heavy chain sequences listed in Table 13. As anon-limiting example, the antibody may be one or more of the light chain sequences listed in Table 13, or variants or fragments thereof.

In some embodiments, the payload region of the AAV particle comprises a nucleic acid sequence encoding a polypeptide comprising a heavy chain and alight chain sequence listed in Table 13, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Table 13, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 13, one or more linkers from Table 2 and a heavy chain sequence from Table 13.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 13, one or more linkers from Table 2, and alight chain sequence from Table 13.

In some embodiments, the payload region comprises a nucleic acid sequence encoding a single heavy chain. As a non-limiting example, the heavy chain is an amino acid sequence or fragment thereof described in Table 13.

Shown in Table 13 are a listing of antibodies and their polynucleotides and/or polypeptides sequences. These sequences may be encoded by or included in the AAV particles of the present disclosure. Variants or fragments of the antibody sequences described in Table 13 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 13. CDRs of the antibodies encoded by the viral genomes of the present disclosure may be 50%, 60%,70%, 80%, 90%, 95% identical to CDRs listed in or incorporated in the sequences of Table 13. Methods of determining CDRs are well known in the art and are described herein. Payload regions may encode antibody variants with one or more heavy chain variable domain (V_{H}) or light chain variable domain (V_{L}) derived from the antibody sequences in Table 13. In some cases, such variants may include bispecific antibodies. Bispecific antibodies encoded by payload regions may comprise variable domain pairs from two different antibodies.

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any pain-associated antibodies, not limited to those described in Table 13, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the pain-associated antibodies as described in International Publication Number WO2017058771, WO2017062016, WO2017062456, WO2017068472, WO2017074013, WO2017075052, WO2017079369, WO2017100193, WO2017102833, WO2017118761, WO2017120344, WO2017123636, WO2017139290, WO2017155990, WO2017156479, WO2017180993, WO2017181031, WO2017181039, WO2017181098, WO2017181139, WO2017189805, WO2017189959, WO2017210278, WO2018009903, WO2018022762, WO2018025982, WO2018039506, WO2018050111, WO2018053029, WO2018081282, WO2018083538, WO2018083645, WO2018102294, WO2018102597, WO2018119246, WO2018124107, WO2018132423, WO2018153262, WO2018157710, WO2018160896, WO2018167322, WO2018182266, WO2018187682, WO2018191414, WO2018204156, WO2018204757, WO2018204871, WO2018206565, WO2018213204, WO2018213679, WO2018215614, WO2018217638, WO2018223051, WO2018237064, WO2019012014, WO2019025847, WO2019028367, WO2019028456, WO2019042282, WO2019057992, WO2019059771, WO2019067293, WO2019067815, WO2019075136, WO2019084438, WO2019087133, WO2019089973, WO2019090069, WO2019090074, WO2019090076, WO2019090078, WO2019090081, WO2019090082, WO2019090085, WO2019090088, WO2019090090, the contents of each of which are herein incorporated by reference in their entirety.

In some embodiments, payloads may encode pain-associated antibodies (or fragments thereof) taught in US Publication Number US20170306013, the contents of each of which are herein incorporated by reference in their entirety. Such embodiments may include antibodies 22D04, 22G08, 22G09, 25A01, 25C01, 25F08, 27A03, 28608, 28CII, 30G01, 32A07, 321304, 32D04, 32E01, 35A06, 35A10, and 35EII, or fragments thereof. In certain embodiments, the payload region encodes antibodies 22D04, 22G08, 22G09, 25A01, 25C01, 25F08, 27A03, 28608, 28CII, 30G01, 32A07, 321304, 32D04, 32E01, 35A06, 35A10, and 35EII, or fragments thereof selected from SEQ ID NO: 330-602 as described in US20170306013A1.

In some embodiments, the payload region of the viral particle comprises one or more nucleic acid sequences encoding one or more of the migraine and pain payload antibody polypeptides listed in Table 10 of U.S. provisional patent application 62/844,433 (MP1-MP564; SEQ ID NO: 19666-20229), the contents of which are herein incorporated by reference in their entirety.

### Antibodies for the Treatment of Ocular Diseases

In some embodiments, the payload region of the AAV particle comprises one or more nucleic acid sequences encoding ocular disease-associated antibodies, variants or fragments thereof.

In some embodiments, the payload region of the AAV particle comprises one or more nucleic acid sequences encoding one or more of the payload antibody polypeptides listed in Tables 14, or variants or fragments thereof.

In some embodiments, the payload region of the AAV particle comprises one or more nucleic acid sequences listed in Table 14, or variants or fragments thereof.

In some embodiments, the payload region of the AAV particle comprises a nucleic acid sequence encoding a payload antibody with at least 50% identity to one or more payload antibody polypeptides listed in Table 14. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 14, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 14, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 14, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 14, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 14, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%,86%, 87%, 88%, 89%, 90%, 91%, 92%, 93%, 94%, 95%, 96%, 97%, 98%, 99%, or 100% identity to the CDRs of one or more of the payload antibody polypeptides listed in Table 14, or variants or fragments thereof.

In some embodiments, the payload antibody has 90% identity to one or more of the antibody polypeptides listed in Table 14, or variants or fragments thereof.

In some embodiments, the payload antibody has 91% identity to one or more of the antibody polypeptides listed in Table 14, or variants or fragments thereof.

In some embodiments, the payload antibody has 92% identity to one or more of the antibody polypeptides listed in Table 14, or variants or fragments thereof.

In some embodiments, the payload antibody has 93% identity to one or more of the antibody polypeptides listed in Table 14, or variants or fragments thereof.

In some embodiments, the payload antibody has 94% identity to one or more of the antibody polypeptides listed in Table 14, or variants or fragments thereof.

In some embodiments, the payload antibody has 95% identity to one or more of the antibody polypeptides listed in Table 14, or variants or fragments thereof.

In some embodiments, the payload antibody has 96% identity to one or more of the antibody polypeptides listed in Table 14, or variants or fragments thereof.

In some embodiments, the payload antibody has 97% identity to one or more of the antibody polypeptides listed in Table 14, or variants or fragments thereof.

In some embodiments, the payload antibody has 98% identity to one or more of the antibody polypeptides listed in Table 14, or variants or fragments thereof.

In some embodiments, the payload antibody has 99% identity to one or more of the antibody polypeptides listed in Table 14, or variants or fragments thereof.

In some embodiments, the payload antibody has 100% identity to one or more of the antibody polypeptides listed in Table 14, or variants or fragments thereof.

In some embodiments, the payload antibody may be variants of any of the antibody polypeptides listed in Table 14, that exclude one or more amino acids designated as “X” or “x” in the described polypeptide sequence, wherein X may represent any amino acid. In some embodiments, the payload nucleic acid sequence may be variants of any of the nucleic acid sequences listed in Table 14, that exclude one or more nucleic acids designated as “n” or “N” in the described nucleic acid sequence, wherein n may represent any nucleic acid.

TABLE 14
Ocular disease antibodies

| | | Type | | |
|---|---|---|---|---|
| Ab ID | Component | (PRT/DNA) | Reference | SEQ ID NO |
| OCL1 | Antibody | PRT | WO2017062649; SEQ ID NO: 2 | 10587 |
| OCL2 | Antibody | PRT | WO2017062649; SEQ ID NO: 5 | 10588 |
| OCL3 | HC | PRT | WO2011117329; SEQ ID NO: 7 | 10589 |
| OCL4 | HC | PRT | WO2011117329; SEQ ID NO: 8 | 10590 |
| OCL5 | HC | PRT | WO2011117329; SEQ ID NO: 12 | 10591 |
| OCL6 | HC | PRT | WO2011117329; SEQ ID NO: 24 | 10592 |
| OCL7 | HC | PRT | WO2011117329; SEQ ID NO: 30 | 10593 |
| OCL8 | HC | PRT | WO2014009465; SEQ ID NO: 21 | 10594 |
| OCL9 | HC | PRT | WO2014009465; SEQ ID NO: 22 | 10595 |
| OCL10 | HC | PRT | WO2014009465; SEQ ID NO: 25 | 10596 |
| OCL11 | HC | PRT | WO2014009465; SEQ ID NO: 26 | 10597 |
| OCL12 | HC | PRT | WO2014009465; SEQ ID NO: 29 | 10598 |
| OCL13 | HC | PRT | WO2014009465; SEQ ID NO: 30 | 10599 |
| OCL14 | HC | PRT | WO2014009465; SEQ ID NO: 33 | 10600 |
| OCL15 | HC | PRT | WO2014009465; SEQ ID NO: 34 | 10601 |
| OCL16 | HC | PRT | WO2014009465; SEQ ID NO: 37 | 10602 |
| OCL17 | HC | PRT | WO2014009465; SEQ ID NO: 39 | 10603 |
| OCL18 | HC | PRT | WO2014009465; SEQ ID NO: 43 | 10604 |
| OCL19 | HC | PRT | WO2014009465; SEQ ID NO: 44 | 10605 |
| OCL20 | HC | PRT | WO2017053807; SEQ ID NO: 48 | 10606 |
| OCL21 | HC | PRT | WO2017053807; SEQ ID NO: 49 | 10607 |
| OCL22 | HC | PRT | WO2017062649; SEQ ID NO: 47 | 10608 |
| OCL23 | LC | PRT | WO2011117329; SEQ ID NO: 5 | 10609 |
| OCL24 | LC | PRT | WO2011117329; SEQ ID NO: 6 | 10610 |
| OCL25 | LC | PRT | WO2011117329; SEQ ID NO: 9 | 10611 |
| OCL26 | LC | PRT | WO2011117329; SEQ ID NO: 10 | 10612 |
| OCL27 | LC | PRT | WO2011117329; SEQ ID NO: 14 | 10613 |
| OCL28 | LC | PRT | WO2011117329; SEQ ID NO: 17 | 10614 |
| OCL29 | LC | PRT | WO2011117329; SEQ ID NO: 18 | 10615 |
| OCL30 | LC | PRT | WO2011117329; SEQ ID NO: 22 | 10616 |
| OCL31 | LC | PRT | WO2011117329; SEQ ID NO: 26 | 10617 |
| OCL32 | LC | PRT | WO2011117329; SEQ ID NO: 31 | 10618 |
| OCL33 | LC | PRT | WO2011117329; SEQ ID NO: 34 | 10619 |
| OCL34 | LC | PRT | WO2011117329; SEQ ID NO: 37 | 10620 |
| OCL35 | LC | PRT | WO2011117329; SEQ ID NO: 40 | 10621 |
| OCL36 | LC | PRT | WO2011117329; SEQ ID NO: 43 | 10622 |
| OCL37 | LC | PRT | WO2011117329; SEQ ID NO: 46 | 10623 |
| OCL38 | LC | PRT | WO2014009465; SEQ ID NO: 24 | 10624 |
| OCL39 | LC | PRT | WO2014009465; SEQ ID NO: 32 | 10625 |
| OCL49 | LC | PRT | WO2017053807; SEQ ID NO: 50 | 10626 |
| OCL41 | LC | PRT | WO2017062649; SEQ ID NO: 48 | 10627 |
| OCL42 | LC | PRT | WO2017062649; SEQ ID NO: 49 | 10628 |
| OCL43 | VH | PRT | WO2011117329; SEQ ID NO: 3 | 10629 |
| OCL44 | VH | PRT | WO2014009465; SEQ ID NO: 7 | 10630 |
| OCL45 | VH | PRT | WO2014009465; SEQ ID NO: 15 | 10631 |
| OCL46 | VH | PRT | WO2017053807; SEQ ID NO: 11 | 10632 |
| OCL47 | VH | PRT | WO2017053807; SEQ ID NO: 33 | 10633 |
| OCL48 | VH | PRT | WO2017053807; SEQ ID NO: 37 | 10634 |
| OCL49 | VH | PRT | WO2017053807; SEQ ID NO: 40 | 10635 |
| OCL50 | VH | PRT | WO2017053807; SEQ ID NO: 42 | 10636 |
| OCL51 | VH | PRT | WO2017079833; SEQ ID NO: 29 | 10637 |
| OCL52 | VH | PRT | WO2018014126; SEQ ID NO: 9 | 10638 |
| OCL53 | VH | PRT | WO2018014126; SEQ ID NO: 14 | 10639 |
| OCL54 | VH | PRT | WO2018014126; SEQ ID NO: 16 | 10640 |
| OCL55 | VH | PRT | WO2018014126; SEQ ID NO: 18 | 10641 |
| OCL56 | VH | PRT | WO2018014126; SEQ ID NO: 20 | 10642 |
| OCL57 | VH | PRT | WO2018014126; SEQ ID NO: 22 | 10643 |
| OCL58 | VH | PRT | WO2018014126; SEQ ID NO: 24 | 10644 |
| OCL59 | VH | PRT | WO2018014126; SEQ ID NO: 26 | 10645 |
| OCL60 | VH | PRT | WO2018014126; SEQ ID NO: 42 | 10646 |
| OCL61 | VH | PRT | WO2018014126; SEQ ID NO: 44 | 10647 |
| OCL62 | VH | PRT | WO201S014126; SEQ ID NO: 46 | 10648 |
| OCL63 | VH | PRT | WO2018014126; SEQ ID NO: 48 | 10649 |
| OCL64 | VH | PRT | WO2018014126; SEQ ID NO: 50 | 10650 |
| OCL65 | VH | PRT | WO2018014126; SEQ ID NO: 52 | 10651 |
| OCL66 | VH | PRT | WO2018014126; SEQ ID NO: 54 | 10652 |
| OCL67 | VH | PRT | WO2018014126; SEQ ID NO: 85 | 10653 |
| OCL68 | VL | PRT | WO2011117329; SEQ ID NO: 2 | 10654 |
| OCL69 | VL | PRT | WO2011117329; SEQ ID NO: 4 | 10655 |
| OCL70 | VL | PRT | WO2014009465; SEQ ID NO: 8 | 10656 |
| OCL71 | VL | PRT | WO2014009465; SEQ ID NO: 16 | 10657 |
| OCL72 | VL | PRT | WO2017053807; SEQ ID NO: 12 | 10658 |
| OCL73 | VL | PRT | WO2017053807; SEQ ID NO: 34 | 10659 |
| OCL74 | VL | PRT | WO2017053807; SEQ ID NO: 35 | 10660 |
| OCL75 | VL | PRT | WO2017053807; SEQ ID NO: 36 | 10661 |
| OCL76 | VL | PRT | WO2017053807; SEQ ID NO: 38 | 10662 |
| OCL77 | VL | PRT | WO2017053807; SEQ ID NO: 41 | 10663 |
| OCL78 | VL | PRT | WO2017053807; SEQ ID NO: 46 | 10664 |
| OCL79 | VL | PRT | WO2017053807; SEQ ID NO: 59 | 10665 |
| OCL80 | VL | PRT | WO2017053807; SEQ ID NO: 60 | 10666 |
| OCL81 | VL | PRT | WO2017079833; SEQ ID NO: 31 | 10667 |
| OCL82 | VL | PRT | WO2018014126; SEQ ID NO: 11 | 10668 |
| OCL83 | VL | PRT | WO2018014126; SEQ ID NO: 28 | 10669 |
| OCL84 | VL | PRT | WO2018014126; SEQ ID NO: 30 | 10670 |
| OCL85 | VL | PRT | WO2018014126; SEQ ID NO: 32 | 10671 |
| OCL86 | VL | PRT | WO2018014126; SEQ ID NO: 34 | 10672 |
| OCL87 | VL | PRT | WO2018014126; SEQ ID NO: 36 | 10673 |
| OCL88 | VL | PRT | WO2018014126; SEQ ID NO: 38 | 10674 |
| OCL89 | VL | PRT | WO2018014126; SEQ ID NO: 40 | 10675 |
| OCL99 | VL | PRT | WO2018014126; SEQ ID NO: 58 | 10676 |
| OCL91 | VL | PRT | WO2018014126; SEQ ID NO: 60 | 10677 |
| OCL92 | VL | PRT | WO2018014126; SEQ ID NO: 64 | 10678 |
| OCL93 | VL | PRT | WO2018014126; SEQ ID NO: 66 | 10679 |
| OCL94 | VL | PRT | WO2018014126; SEQ ID NO: 68 | 10680 |
| OCL95 | VL | PRT | WO2018014126; SEQ ID NO: 87 | 10681 |
| OCL96 | VL | PRT | WO2018014126; SEQ ID NO: 89 | 10682 |

In some embodiments, the payload region of the AAV particle comprises a nucleic acid sequence with at least 50% identity to one or more nucleic acid sequences listed in Table 14, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%: 71%, 72%, 73%, 74%, 75%, 76%, 77%, 78%, 79%, 80%, 81%, 82%, 83%, 84%, 85%, 86%, 87%, 88%, 89%, 90%, 91% 92% 93%. 94%, 95%, 96%, 97%, 98%, 99%, or 100% identity to one or more nucleic acid sequences listed in Table 14, or variants or fragments thereof.

In some embodiments, the payload nucleic acid sequence has 90% identity to one or more of the nucleic acid sequences listed in Table 14, or variants or fragments thereof.

In some embodiments, the payload nucleic acid sequence has 91% identity to one or more of the nucleic acid sequences listed in Table 14, or variants or fragments thereof.

In some embodiments, the payload nucleic acid sequence has 92% identity to one or more of the nucleic acid sequences listed in Table 14, or variants or fragments thereof.

In some embodiments, the payload nucleic acid sequence has 93% identity to one or more of the nucleic acid sequences listed in Table 14, or variants or fragments thereof.

In some embodiments, the payload nucleic acid sequence has 94% identity to one or more of the nucleic acid sequences listed in Table 14, or variants or fragments thereof.

In some embodiments, the payload nucleic acid sequence has 95% identity to one or more of the nucleic acid sequences listed in Table 14, or variants or fragments thereof.

In some embodiments, the payload nucleic acid sequence has 96% identity to one or more of the nucleic acid sequences listed in Table 14, or variants or fragments thereof.

In some embodiments, the payload nucleic acid sequence has 97% identity to one or more of the nucleic acid sequences listed in Table 14, or variants or fragments thereof.

In some embodiments, the payload nucleic acid sequence has 98% identity to one or more of the nucleic acid sequences listed in Table 14, or variants or fragments thereof.

In some embodiments, the payload nucleic acid sequence has 99% identity to one or more of the nucleic acid sequences listed in Table 14, or variants or fragments thereof.

In some embodiments, the payload nucleic acid sequence has 100% identity to one or more of the nucleic acid sequences listed in Table 14, or variants or fragments thereof.

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 14, or variants or fragments thereof. As another non-limiting example, the antibody may be one or more of the heavy chain sequences listed in Table 14. As a non-limiting example, the antibody may be one or more of the light chain sequences listed in Table 14, or variants or fragments thereof.

In some embodiments, the payload region of the AAV particle comprises a nucleic acid sequence encoding a polypeptide comprising a heavy chain and alight chain sequence listed in Table 14, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and alight chain sequence listed in Table 14, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 14, one or more linkers from Table 2 and a heavy chain sequence from Table 14.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 14, one or more linkers from Table 2, and alight chain sequence from Table 14.

In some embodiments, the payload region comprises a nucleic acid sequence encoding a single heavy chain. As a non-limiting example, the heavy chain is an amino acid sequence or fragment thereof described in Table 14.

Shown in Table 14 are a listing of antibodies and their polynucleotides and/or polypeptides sequences. These sequences may be encoded by or included in the AAV particles of the present disclosure. Variants or fragments of the antibody sequences described in Table 14 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 14. CDRs of the antibodies encoded by the viral genomes of the present disclosure may be 50%, 60%,70%, 80%, 90%, 95% identical to CDRs listed in or incorporated in the sequences of Table 14. Methods of determining CDRs are well known in the art and are described herein. Payload regions may encode antibody variants with one or more heavy chain variable domain (V_{H}) or light chain variable domain (V_{L}) derived from the antibody sequences in Table 14. In some cases, such variants may include bispecific antibodies. Bispecific antibodies encoded by payload regions may comprise variable domain pairs from two different antibodies.

In some embodiments, the AAV particles may comprise a heavy and alight chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any ocular disease-associated antibodies, not limited to those described in Table 14, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the ocular disease-associated antibodies as described in International Publication Number WO2017048614, WO2017053807, WO2017054086, WO2017062649, WO2017062820, WO2017062952, WO2017067423, WO2017072325, WO2017075173, WO2017075212, WO2017075252, WO2017075259, WO2017079443, WO2017083488, WO2017083750, WO2017091719, WO2017100540, WO2017106236, WO2017117430, WO2017117464, WO2017120344, WO2017123646, WO2017124002, WO2017127833, WO2017129064, WO2017134301, WO2017134302, WO2017134305, WO2017134306, WO2017136350, WO2017136355, WO2017136549, WO2017137542, WO2017147293, WO2017149538, WO2017152102, WO2017153567, WO2017156488, WO2017176864, WO2017177169, WO2017180530, WO2017180936, WO2017180993, WO2017181021, WO2017181031, WO2017181039, WO2017186928, WO2017188570, WO2017189959, WO2017189963, WO2017189964, WO2017192538, WO2017194568, WO2017194782, WO2017194783, WO2017197331, WO2017197376, WO2017211731, WO2017214706, WO2017215524, WO2017218977, WO2017221128, WO2018005904, WO2018007314, WO2018017714, WO2018027329, WO2018039626, WO2018045325, WO2018053434, WO2018064190, WO2018070390, WO2018073680, WO2018077893, WO2018089305, WO2018102594, WO2018124582, WO2018127519, WO2018127586, WO2018127791, WO2018129404, WO2018129524, WO2018141964, WO2018144999, WO2018146074, WO2018158658, WO2018158727, WO2018160538, WO2018160896, WO2018161092, WO2018164385, WO2018165362, WO2018169948, WO2018175476, WO2018175752, WO2018175788, WO2018175988, WO2018178307, WO2018182266, WO2018191548, WO2018195283, WO2018195912, WO2018200620, WO2018204868, WO2018208625, WO2018208709, WO2018210898, WO2018212714, WO2018213260, WO2018213592, WO2018217976, WO2018217988, WO2018218083, WO2018218215, WO2018218240, WO2018220216, WO2018224630, WO2018226578, WO2019003074, WO2019015673, WO2019028051, WO2019028427, WO2019032661, WO2019032662, WO2019032663, WO2019042226, WO2019054819, WO2019055842, WO2019060653, WO2019062871, WO2019067540, WO2019067682, WO2019075136, WO2019075270, WO2019079249, WO2019079809, WO2019085804, WO2019086580, WO2019087087, WO2019089973, WO2019090069, WO2019090074, WO2019090076, WO2019090078, WO2019090081, WO2019090082, WO2019090085, WO2019090088, WO2019090090, WO2019090110, WO2019093807, WO2019094578, and WO2019094700, the contents of each of which are herein incorporated by reference in their entirety.

In some embodiments, payloads of the present disclosure may encode one or more of the ocular associated antibodies (or fragments thereof) taught in International Patent Publication Numbers WO2017053807, WO2010040508, WO2014009465, and/or WO2011117329 (the contents of each of which are herein incorporated by reference in their entirety). In some embodiments, the ocular associated antibodies may be Faricimab and/or Vanucizumab described in International Patent Publication Numbers WO2017053807, WO2010040508, WO2014009465, and/or WO2011117329.

In some embodiments, the payload region of the viral particle comprises one or more nucleic acid sequences encoding one or more of the ocular disease payload antibody polypeptides listed in Table 11 of US provisional patent application 623844,433 (OC1-OC676; SEQ ID NO: 20230-20905), the contents of which are herein incorporated by reference in their entirety.

### Antibodies for the Treatment of Excretory and Kidney Diseases

In some embodiments, the payload region of the AAV particle comprises one or more nucleic acid sequences encoding ocular disease-associated antibodies, variants or fragments thereof.

In some embodiments, the payload region of the AAV particle comprises one or more nucleic acid sequences encoding one or more of the payload antibody polypeptides listed in Tables 15, or variants or fragments thereof.

In some embodiments, the payload region of the AAV particle comprises one or more nucleic acid sequences listed in Table 15, or variants or fragments thereof.

In some embodiments, the payload region of the AAV particle comprises a nucleic acid sequence encoding a payload antibody with at least 50% identity to one or more payload antibody polypeptides listed in Table 15. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 15, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 15, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 15, or variants or fragments thereof.

In some embodiments, the heavy chain of the encoded antibody polypeptide may have 50%, 51%, 52%,53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 15, or variants or fragments thereof.

In some embodiments, the light chain of the encoded antibody polypeptide may have 50%, 51%, 52%, 53%,54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 15, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%,87%,88%, 89%, 90%, 91%, 92%, 93%, 94%, 95%, 96%, 97%, 98%, 99%, or 100% identity to the CDRs of one or more of the payload antibody polypeptides listed in Table 15, or variants or fragments thereof.

In some embodiments, the payload antibody has 90% identity to one or more of the antibody polypeptides listed in Table 15, or variants or fragments thereof.

In some embodiments, the payload antibody has 91% identity to one or more of the antibody polypeptides listed in Table 15, or variants or fragments thereof.

In some embodiments, the payload antibody has 92% identity to one or more of the antibody polypeptides listed in Table 15, or variants or fragments thereof.

In some embodiments, the payload antibody has 93% identity to one or more of the antibody polypeptides listed in Table 15, or variants or fragments thereof.

In some embodiments, the payload antibody has 94% identity to one or more of the antibody polypeptides listed in Table 15, or variants or fragments thereof.

In some embodiments, the payload antibody has 95% identity to one or more of the antibody polypeptides listed in Table 15, or variants or fragments thereof.

In some embodiments, the payload antibody has 96% identity to one or more of the antibody polypeptides listed in Table 15, or variants or fragments thereof.

In some embodiments, the payload antibody has 97% identity to one or more of the antibody polypeptides listed in Table 15, or variants or fragments thereof.

In some embodiments, the payload antibody has 98% identity to one or more of the antibody polypeptides listed in Table 15, or variants or fragments thereof.

In some embodiments, the payload antibody has 99% identity to one or more of the antibody polypeptides listed in Table 15, or variants or fragments thereof.

In some embodiments, the payload antibody has 100% identity to one or more of the antibody polypeptides listed in Table 15, or variants or fragments thereof.

In some embodiments, the payload antibody may be variants of any of the antibody polypeptides listed in Table 15, that exclude one or more amino acids designated as “X” or “x” in the described polypeptide sequence, wherein X may represent any amino acid. In some embodiments, the payload nucleic acid sequence may be variants of any of the nucleic acid sequences listed in Table 15, that exclude one or more nucleic acids designated as “n” or “N” in the described nucleic acid sequence, wherein n may represent any nucleic acid.

TABLE 15
Excretory and Kidney disease antibodies

| | | Type | | |
|---|---|---|---|---|
| Ab ID | Component | (PRT/DNA) | Reference | SEQ ID NO |
| EKD1 | HC | PRT | WO2018227063; SEQ ID NO: 34 | 10683 |
| EKD2 | HC | PRT | WO2018227063; SEQ ID NO: 36 | 10684 |
| EKD3 | HC | PRT | WO2018227063; SEQ ID NO: 37 | 10685 |
| EKD4 | HC | PRT | WO2018227063; SEQ ID NO: 45 | 10686 |
| EKD5 | HC | PRT | WO2018227063; SEQ ID NO: 51 | 10687 |
| EKD6 | HC | PRT | WO2018227063; SEQ ID NO: 57 | 10688 |
| EKD7 | HC | PRT | WO2018227063; SEQ ID NO: 72 | 10689 |
| EKD8 | HC | PRT | WO2018227063; SEQ ID NO: 75 | 10690 |
| EKD9 | HC | PRT | WO2018227063; SEQ ID NO: 78 | 10691 |
| EKD10 | HC | PRT | WO2018227063; SEQ ID NO: 81 | 10692 |
| EKD11 | HC | PRT | WO2018227063; SEQ ID NO: 84 | 10693 |
| EKD12 | HC | PRT | WO2018227063; SEQ ID NO: 87 | 10694 |
| EKD13 | HC | PRT | WO2018227063; SEQ ID NO: 90 | 10695 |
| EKD14 | HC | PRT | WO2018227063; SEQ ID NO: 118 | 10696 |
| EKD15 | HC | PRT | WO2018227063; SEQ ID NO: 121 | 10697 |
| EKD16 | HC | PRT | WO2018227063; SEQ ID NO: 134 | 10698 |
| EKD17 | HC | PRT | WO2018227063; SEQ ID NO: 135 | 10699 |
| EKD18 | HC | PRT | WO2018227063; SEQ ID NO: 136 | 10700 |
| EKD19 | HC | PRT | WO2018227063; SEQ ID NO: 137 | 10701 |
| EKD20 | HC | PRT | WO2018227063; SEQ ID NO: 138 | 10702 |
| EKD21 | HC | PRT | WO2018227063; SEQ ID NO: 139 | 10703 |
| EKD22 | HC | PRT | WO2018227063; SEQ ID NO: 140 | 10704 |
| EKD23 | LC | PRT | WO2018227063; SEQ ID NO: 38 | 10705 |
| EKD24 | LC | PRT | WO2018227063; SEQ ID NO: 42 | 10706 |
| EKD25 | LC | PRT | WO2018227063; SEQ ID NO: 48 | 10707 |
| EKD26 | LC | PRT | WO2018227063; SEQ ID NO: 54 | 10708 |
| EKD27 | LC | PRT | WO2018227063; SEQ ID NO: 60 | 10709 |
| EKD28 | LC | PRT | WO2018227063; SEQ ID NO: 63 | 10710 |
| EKD29 | LC | PRT | WO2018227063; SEQ ID NO: 66 | 10711 |
| EKD30 | LC | PRT | WO2018227063; SEQ ID NO: 69 | 10712 |
| EKD31 | LC | PRT | WO2018227063; SEQ ID NO: 93 | 10713 |
| EKD32 | LC | PRT | WO2018227063; SEQ ID NO: 96 | 10714 |
| EKD33 | LC | PRT | WO2018227063; SEQ ID NO: 98 | 10715 |
| EKD34 | LC | PRT | WO2018227063; SEQ ID NO: 100 | 10716 |
| EKD35 | LC | PRT | WO2018227063; SEQ ID NO: 102 | 10717 |
| EKD36 | LC | PRT | WO2018227063; SEQ ID NO: 104 | 10718 |
| EKD37 | LC | PRT | WO2018227063; SEQ ID NO: 106 | 10719 |
| EKD38 | LC | PRT | WO2018227063; SEQ ID NO: 108 | 10720 |
| EKD39 | LC | PRT | WO2018227063; SEQ ID NO: 110 | 10721 |
| EKD40 | LC | PRT | WO2018227063; SEQ ID NO: 112 | 10722 |
| EKD41 | LC | PRT | WO2018227063; SEQ ID NO: 114 | 10723 |
| EKD42 | LC | PRT | WO2018227063; SEQ ID NO: 141 | 10724 |
| EKD43 | VH | PRT | WO2018227063; SEQ ID NO: 32 | 10725 |
| EKD44 | VH | PRT | WO2018227063; SEQ ID NO: 43 | 10726 |
| EKD45 | VH | PRT | WO2018227063; SEQ ID NO: 49 | 10727 |
| EKD46 | VH | PRT | WO2018227063; SEQ ID NO: 55 | 10728 |
| EKD47 | VH | PRT | WO2018227063; SEQ ID NO: 70 | 10729 |
| EKD48 | VH | PRT | WO2018227063; SEQ ID NO: 73 | 10730 |
| EKD49 | VH | PRT | WO2018227063; SEQ ID NO: 76 | 10731 |
| EKD50 | VH | PRT | WO2018227063; SEQ ID NO: 79 | 10732 |
| EKD51 | VH | PRT | WO2018227063; SEQ ID NO: 82 | 10733 |
| EKD52 | VH | PRT | WO2018227063; SEQ ID NO: 85 | 10734 |
| EKD53 | VH | PRT | WO2018227063; SEQ ID NO: 88 | 10735 |
| EKD54 | VH | PRT | WO2018227063; SEQ ID NO: 115 | 10736 |
| EKD55 | VH | PRT | WO2018227063; SEQ ID NO: 119 | 10737 |
| EKD56 | VH | PRT | WO2018227063; SEQ ID NO: 126 | 10738 |
| EKD57 | VH | PRT | WO2018227063; SEQ ID NO: 127 | 10739 |
| EKD58 | VH | PRT | WO2018227063; SEQ ID NO: 128 | 10740 |
| EKD59 | VH | PRT | WO2018227063; SEQ ID NO: 129 | 10741 |
| EKD60 | VH | PRT | WO20182270S3; SEQ ID NO: 130 | 10742 |
| EKD61 | VH | PRT | WO2018227063; SEQ ID NO: 131 | 10743 |
| EKD62 | VH | PRT | WO2018227063; SEQ ID NO: 132 | 10744 |
| EKD63 | VL | PRT | WO2018227063; SEQ ID NO: 39 | 10745 |
| EKD64 | VL | PRT | WO2018227063; SEQ ID NO: 46 | 10746 |
| EKD65 | VL | PRT | WO2018227063; SEQ ID NO: 52 | 10747 |
| EKD66 | VL | PRT | WO2018227063; SEQ ID NO: 58 | 10748 |
| EKD67 | VL | PRT | WO2018227063; SEQ ID NO: 61 | 10749 |
| EKD68 | VL | PRT | WO2018227063; SEQ ID NO: 64 | 10750 |
| EKD69 | VL | PRT | WO2018227063; SEQ ID NO: 67 | 10751 |
| EKD70 | VL | PRT | WO2018227063; SEQ ID NO: 91 | 10752 |
| EKD71 | VL | PRT | WO2018227063; SEQ ID NO: 94 | 10753 |
| EKD72 | VL | PRT | WO2018227063; SEQ ID NO: 97 | 10754 |
| EKD73 | VL | PRT | WO2018227063; SEQ ID NO: 99 | 10755 |
| EKD74 | VL | PRT | WO2018227063; SEQ ID NO: 101 | 10756 |
| EKD75 | VL | PRT | WO2018227063; SEQ ID NO: 103 | 10757 |
| EKD76 | VL | PRT | WO2018227063; SEQ ID NO: 105 | 10758 |
| EKD77 | VL | PRT | WO2018227063; SEQ ID NO: 107 | 10759 |
| EKD78 | VL | PRT | WO2018227063; SEQ ID NO: 109 | 10760 |
| EKD79 | VL | PRT | WO2018227063; SEQ ID NO: 111 | 10761 |
| EKD80 | VL | PRT | WO2018227063; SEQ ID NO: 113 | 10762 |
| EKD81 | VL | PRT | WO2018227063; SEQ ID NO: 133 | 10763 |

Antibodies for the Treatment of Multiple Specific Diseases and/or Targets

In some embodiments, the payload region of the AAV particle comprises one or more nucleic add sequences encoding multiple specific disease- and/or target-associated antibodies, variants or fragments thereof.

In some embodiments, the payload region of the AAV particle comprises one or more nucleic add sequences encoding one or more of the payload antibody polypeptides listed in Table 16, or variants or fragments thereof. As used herein, “antibody polynucleotide” refers to a nucleic acid sequence encoding an antibody polypeptide.

In some embodiments, the payload region of the AAV particle comprises one or more nucleic add sequences listed in Table 16, or variants or fragments thereof.

In some embodiments, the payload region of the AAV particle comprises a nucleic acid sequence encoding a payload antibody with at least 50% identity to one or more payload antibody polypeptides listed in Table 16. The encoded antibody polypeptide may have 50%: 51%, 52%, 53%, 54%, 55%, 56%, 57%, 58%, 59%, 60%, 61%, 62%, 63%, 64%, 65%, 66%,697%, 68%, 69%, 70%, 71%, 72%, 73%, 74%, 75%, 76%, 77%, 78%, 79%,80%,81%, 82%,83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 16, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%,64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 16, or variants or fragments thereof.

In some embodiments, the variable region sequence(s) of the encoded antibody polypeptide may have 50%,51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 16, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%,77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 16, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 16, or variants or fragments thereof.

In some embodiments, the CDR region of the encoded antibody polypeptide may have 50%, 51%, 52%,53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%,90%,91%, 92%,93%,94%, 95%, 96%,97%,98%, 99%, or 100% identity to the CDRs of one or more of the payload antibody polypeptides listed in Table 16, or variants or fragments thereof.

In some embodiments, the payload antibody has 90% identity to one or more of the antibody polypeptides listed in Table 16, or variants or fragments thereof.

In some embodiments, the payload antibody has 91% identity to one or more of the antibody polypeptides listed in Table 16, or variants or fragments thereof.

In some embodiments, the payload antibody has 92% identity to one or more of the antibody polypeptides listed in Table 16, or variants or fragments thereof.

In some embodiments, the payload antibody has 93% identity to one or more of the antibody polypeptides listed in Table 16, or variants or fragments thereof.

In some embodiments, the payload antibody has 94% identity to one or more of the antibody polypeptides listed in Table 16, or variants or fragments thereof.

In some embodiments, the payload antibody has 95% identity to one or more of the antibody polypeptides listed in Table 16, or variants or fragments thereof.

In some embodiments, the payload antibody has 96% identity to one or more of the antibody polypeptides listed in Table 16, or variants or fragments thereof.

In some embodiments, the payload antibody has 97% identity to one or more of the antibody polypeptides listed in Table 16, or variants or fragments thereof.

In some embodiments, the payload antibody has 98% identity to one or more of the antibody polypeptides listed in Table 16, or variants or fragments thereof.

In some embodiments, the payload antibody has 99% identity to one or more of the antibody polypeptides listed in Table 16, or variants or fragments thereof.

In some embodiments, the payload antibody has 100% identity to one or more of the antibody polypeptides listed in Table 16, or variants or fragments thereof.

In some embodiments, the payload region of the AAV particle comprises a nucleic acid sequence with at least 50% identity to one or more nucleic acid sequences listed in Table 16, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%,67%,68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 16, or variants or fragments thereof.

In some embodiments, the payload nucleic acid sequence has 90% identity to one or more of the nucleic acid sequences listed in Table 16, or variants or fragments thereof.

In some embodiments, the payload nucleic acid sequence has 91% identity to one or more of the nucleic acid sequences listed in Table 16, or variants or fragments thereof.

In some embodiments, the payload nucleic acid sequence has 92% identity to one or more of the nucleic acid sequences listed in Table 16, or variants or fragments thereof.

In some embodiments, the payload nucleic acid sequence has 93% identity to one or more of the nucleic acid sequences listed in Table 16, or variants or fragments thereof.

In some embodiments, the payload nucleic acid sequence has 94% identity to one or more of the nucleic acid sequences listed in Table 16, or variants or fragments thereof.

In some embodiments, the payload nucleic acid sequence has 95% identity to one or more of the nucleic acid sequences listed in Table 16, or variants or fragments thereof.

In some embodiments, the payload nucleic acid sequence has 96% identity to one or more of the nucleic acid sequences listed in Table 16, or variants or fragments thereof.

In some embodiments, the payload nucleic acid sequence has 97% identity to one or more of the nucleic acid sequences listed in Table 16, or variants or fragments thereof.

In some embodiments, the payload nucleic acid sequence has 98% identity to one or more of the nucleic acid sequences listed in Table 16, or variants or fragments thereof.

In some embodiments, the payload nucleic acid sequence has 99% identity to one or more of the nucleic acid sequences listed in Table 16, or variants or fragments thereof.

In some embodiments, the payload nucleic acid sequence has 100% identity to one or more of the nucleic acid sequences listed in Table 16, or variants or fragments thereof.

In some embodiments, the payload antibody may be variants of any of the antibody polypeptides listed in Table 16, that exclude one or more amino acids designated as “X” or “x” in the described polypeptide sequence, wherein X may represent any amino acid. In some embodiments, the payload nucleic acid sequence may be variants of any of the nucleic acid sequences listed in Table 16, that exclude one or more nucleic acids designated as “n” or “N” in the described nucleic acid sequence, wherein n may represent any nucleic acid.

TABLE 16
Multiple specific disease and/or target antibodies

| | | Type | | |
|---|---|---|---|---|
| Ab ID | Component | (PRT/DNA) | Reference | SEQ ID NO. |
| MSP1 | HC | PRT | U.S. Pat. No. 9,080,177; SEQ ID NO: 19 | 10764 |
| MSP2 | HC | PRT | U.S. Pat. No. 9,080,177; SEQ ID NO: 15 | 10765 |
| MSP3 | HC | PRT | U.S. Pat. No. 9,080,177; SEQ ID NO: 13 | 10766 |
| MSP4 | HC | PRT | U.S. Pat. No. 9,080,177; SEQ ID NO: 17 | 10767 |
| MSP5 | HC | PRT | U.S. Pat. No. 9,080,177; SEQ ID NO: 23 | 10768 |
| MSP6 | HC | PRT | U.S. Pat. No. 9,080,177; SEQ ID NO: 21 | 10769 |
| MSP7 | HC | PRT | U.S. Pat. No. 9,080,177; SEQ ID NO: 12 | 10770 |
| MSP8 | LC | PRT | U.S. Pat. No. 9,080,177; SEQ ID NO: 18 | 10771 |
| MSP9 | LC | PRT | U.S. Pat. No. 9,080,177; SEQ ID NO: 20 | 10772 |
| MSP10 | LC | PRT | U.S. Pat. No. 9,080,177; SEQ ID NO: 11 | 10773 |
| MSP11 | LC | PRT | U.S. Pat. No. 9,080,177; SEQ ID NO: 24 | 10774 |
| MSP12 | LC | PRT | U.S. Pat. No. 9,080,177; SEQ ID NO: 22 | 10775 |
| MSP13 | LC | PRT | U.S. Pat. No. 9,080,177; SEQ ID NO: 16 | 10776 |
| MSP14 | LC | PRT | U.S. Pat. No. 9,080,177; SEQ ID NO: 14 | 10777 |
| MSP15 | HC | PRT | WO2017058771; SEQ ID NO: 68 | 10778 |
| MSP16 | LC | PRT | WO2017058771; SEQ ID NO: 8 | 10779 |
| MSP17 | LC | PRT | WO2017058771; SEQ ID NO: 184 | 10780 |
| MSP18 | LC | PRT | WO2017058771; SEQ ID NO: 338 | 10781 |
| MSP19 | Mab | PRT | WO2017058771; SEQ ID NO: 358 | 10782 |
| MSP20 | Mab | PRT | WO2017058771; SEQ ID NO: 359 | 10783 |
| MSP21 | Mab | PRT | WO2017058771; SEQ ID NO: 360 | 10784 |
| MSP22 | Mab | PRT | WO2017058771; SEQ ID NO: 361 | 10785 |
| MSP23 | Mab | PRT | WO2017058771; SEQ ID NO: 362 | 10786 |
| MSP24 | Mab | PRT | WO2017058771; SEQ ID NO: 363 | 10787 |
| MSP25 | Mab | PRT | WO2017058771; SEQ ID NO: 365 | 10788 |
| MSP26 | Mab | PRT | WO2017058771; SEQ ID NO: 366 | 10789 |
| MSP27 | Mab | PRT | WO2017058771; SEQ ID NO: 367 | 10790 |
| MSP28 | Mab | PRT | WO2017058771; SEQ ID NO: 368 | 10791 |
| MSP29 | Mab | PRT | WO2017058771; SEQ ID NO: 369 | 10792 |
| MSP30 | Mab | PRT | WO2017058771; SEQ ID NO: 370 | 10793 |
| MSP31 | Mab | PRT | WO2017058771; SEQ ID NO: 371 | 10794 |
| MSP32 | Mab | PRT | WO2017058771; SEQ ID NO: 372 | 10795 |
| MSP33 | Mab | PRT | WO2017058771; SEQ ID NO: 373 | 10796 |
| MSP34 | Mab | PRT | WO2017058771; SEQ ID NO: 374 | 10797 |
| MSP35 | Mab | PRT | WO2017058771; SEQ ID NO: 375 | 10798 |
| MSP36 | Mab | PRT | WO2017058771; SEQ ID NO: 376 | 10799 |
| MSP37 | Mab | PRT | WO2017058771; SEQ ID NO: 377 | 10800 |
| MSP38 | Mab | PRT | WO2017058771; SEQ ID NO: 378 | 10801 |
| MSP39 | Mab | PRT | WO2017058771; SEQ ID NO: 379 | 10802 |
| MSP40 | Mab | PRT | WO2017058771; SEQ ID NO: 380 | 10803 |
| MSP41 | Mab | PRT | WO2017058771; SEQ ID NO: 381 | 10804 |
| MSP42 | Mab | PRT | WO2017058771; SEQ ID NO: 382 | 10805 |
| MSP43 | Mab | PRT | WO2017058771; SEQ ID NO: 383 | 10806 |
| MSP44 | Mab | PRT | WO2017058771; SEQ ID NO: 384 | 10807 |
| MSP45 | Mab | PRT | WO2017058771; SEQ ID NO: 385 | 10808 |
| MSP46 | Mab | PRT | WO2017058771; SEQ ID NO: 386 | 10809 |
| MSP47 | Mab | PRT | WO2017058771; SEQ ID NO: 387 | 10810 |
| MSP48 | Mab | PRT | WO2017058771; SEQ ID NO: 388 | 10811 |
| MSP49 | Mab | PRT | WO2017058771; SEQ ID NO: 389 | 10812 |
| MSP50 | Mab | PRT | WO2017058771; SEQ ID NO: 390 | 10813 |
| MSP51 | Mab | PRT | WO2017058771; SEQ ID NO: 392 | 10814 |
| MSP52 | Mab | PRT | WO2017058771; SEQ ID NO: 393 | 10815 |
| MSP53 | Mab | PRT | WO2017058771; SEQ ID NO: 394 | 10816 |
| MSP54 | Mab | PRT | WO2017058771; SEQ ID NO: 395 | 10817 |
| MSP55 | Mab | PRT | WO2017058771; SEQ ID NO: 396 | 10818 |
| MSP56 | Mab | PRT | WO2017058771; SEQ ID NO: 398 | 10819 |
| MSP57 | Mab | PRT | WO2017058771; SEQ ID NO: 399 | 10820 |
| MSP58 | Mab | PRT | WO2017058771; SEQ ID NO: 406 | 10821 |
| MSP59 | Mab | PRT | WO2017058771; SEQ ID NO: 402 | 10822 |
| MSP60 | Mab | PRT | WO2017058771; SEQ ID NO: 403 | 10823 |
| MSP61 | Mab | PRT | WO2017058771; SEQ ID NO: 404 | 10824 |
| MSP62 | Mab | PRT | WO2017058771; SEQ ID NO: 405 | 10825 |
| MSP63 | Mab | PRT | WO2017058771; SEQ ID NO: 406 | 10826 |
| MSP64 | Mab | PRT | WO2017058771; SEQ ID NO: 407 | 10827 |
| MSP65 | Mab | PRT | WO2017058771; SEQ ID NO: 408 | 10828 |
| MSP66 | Mab | PRT | WO2017058771; SEQ ID NO: 409 | 10829 |
| MSP67 | Mab | PRT | WO2017058771; SEQ ID NO: 410 | 10830 |
| MSP68 | Mab | PRT | WO2017058771; SEQ ID NO: 411 | 10831 |
| MSP69 | Mab | PRT | WO2017058771; SEQ ID NO: 412 | 10832 |
| MSP70 | Mab | PRT | WO2017058771; SEQ ID NO: 413 | 10833 |
| MSP71 | Mab | PRT | WO2017058771; SEQ ID NO: 415 | 10834 |
| MSP72 | Mab | PRT | WO2017058771; SEQ ID NO: 416 | 10835 |
| MSP73 | Mab | PRT | WO2017058771; SEQ ID NO: 417 | 10836 |
| MSP74 | Mab | PRT | WO2017058771; SEQ ID NO: 418 | 10837 |
| MSP75 | Mab | PRT | WO2017058771; SEQ ID NO: 419 | 10838 |
| MSP76 | Mab | PRT | WO2017058771; SEQ ID NO: 420 | 10839 |
| MSP77 | Mab | PRT | WO2017058771; SEQ ID NO: 421 | 10840 |
| MSP78 | Mab | PRT | WO2017058771; SEQ ID NO: 422 | 10841 |
| MSP79 | Mab | PRT | WO2017058771; SEQ ID NO: 424 | 10842 |
| MSP80 | Mab | PRT | WO2017058771; SEQ ID NO: 425 | 10843 |
| MSP81 | Mab | PRT | WO2017058771; SEQ ID NO: 426 | 10844 |
| MSP82 | Mab | PRT | WO2017058771; SEQ ID NO: 427 | 10845 |
| MSP83 | Mab | PRT | WO2017058771; SEQ ID NO: 428 | 10846 |
| MSP84 | Mab | PRT | WO2017058771; SEQ ID NO: 429 | 10847 |
| MSP85 | Mab | PRT | WO2017058771; SEQ ID NO: 430 | 10848 |
| MSP86 | Mab | PRT | WO2017058771; SEQ ID NO: 431 | 10849 |
| MSP87 | Mab | PRT | WO2017058771; SEQ ID NO: 432 | 10850 |
| MSP88 | VH | PRT | WO2017058771; SEQ ID NO: 2 | 10851 |
| MSP89 | VH | PRT | WO2017058771; SEQ ID NO: 10 | 10852 |
| MSP90 | VH | PRT | WO2017058771; SEQ ID NO: 26 | 10853 |
| MSP91 | VH | PRT | WO2017058771; SEQ ID NO: 34 | 10854 |
| MSP92 | VH | PRT | WO2017058771; SEQ ID NO: 42 | 10855 |
| MSP93 | VH | PRT | WO2017058771; SEQ ID NO: 50 | 10856 |
| MSP94 | VH | PRT | WO2017058771; SEQ ID NO: 62 | 10857 |
| MSP95 | VH | PRT | WO2017058771; SEQ ID NO: 74 | 10858 |
| MSP96 | VH | PRT | WO2017058771; SEQ ID NO: 90 | 10859 |
| MSP97 | VH | PRT | WO2017058771; SEQ ID NO: 106 | 10860 |
| MSP98 | VH | PRT | WO2017058771; SEQ ID NO: 138 | 10861 |
| MSP99 | VH | PRT | WO2017058771; SEQ ID NO: 146 | 10862 |
| MSP100 | VH | PRT | WO2017058771; SEQ ID NO: 154 | 10863 |
| MSP101 | VH | PRT | WO2017058771; SEQ ID NO: 162 | 10864 |
| MSP102 | VH | PRT | WO2017058771; SEQ ID NO: 170 | 10865 |
| MSP103 | VH | PRT | WO2017058771; SEQ ID NO: 178 | 10866 |
| MSP104 | VH | PRT | WO2017058771; SEQ ID NO: 186 | 10867 |
| MSP105 | VH | PRT | WO2017058771; SEQ ID NO: 194 | 10868 |
| MSP106 | VH | PRT | WO2017058771; SEQ ID NO: 202 | 10869 |
| MSP107 | VH | PRT | WO2017058771; SEQ ID NO: 210 | 10870 |
| MSP108 | VH | PRT | WO2017058771; SEQ ID NO: 218 | 10871 |
| MSP109 | VH | PRT | WO2017058771; SEQ ID NO: 250 | 10872 |
| MSP110 | VH | PRT | WO2017058771; SEQ ID NO: 258 | 10873 |
| MSP111 | VH | PRT | WO2017058771; SEQ ID NO: 266 | 10874 |
| MSP112 | VH | PRT | WO2017058771; SEQ ID NO: 339 | 10875 |
| MSP113 | VH | PRT | WO2017058771; SEQ ID NO: 340 | 10876 |
| MSP114 | VH | PRT | WO2017058771; SEQ ID NO: 341 | 10877 |
| MSP115 | VL | PRT | WO2017058771; SEQ ID NO: 14 | 10878 |
| MSP116 | VL | PRT | WO2017058771; SEQ ID NO: 30 | 10879 |
| MSP117 | VL | PRT | WO2017058771; SEQ ID NO: 38 | 10880 |
| MSP118 | VL | PRT | WO2017058771; SEQ ID NO: 46 | 10881 |
| MSP119 | VL | PRT | WO2017058771; SEQ ID NO: 54 | 10882 |
| MSP120 | VL | PRT | WO2017058771; SEQ ID NO: 66 | 10883 |
| MSP121 | VL | PRT | WO2017058771; SEQ ID NO: 78 | 10884 |
| MSP122 | VL | PRT | WO2017058771; SEQ ID NO: 86 | 10885 |
| MSP123 | VL | PRT | WO2017058771; SEQ ID NO: 98 | 10886 |
| MSP124 | VL | PRT | WO2017058771; SEQ ID NO: 102 | 10887 |
| MSP125 | VL | PRT | WO2017058771; SEQ ID NO: 110 | 10888 |
| MSP126 | VL | PRT | WO2017058771; SEQ ID NO: 118 | 10889 |
| MSP127 | VL | PRT | WO2017058771; SEQ ID NO: 126 | 10890 |
| MSP128 | VL | PRT | WO2017058771; SEQ ID NO: 142 | 10891 |
| MSP129 | VL | PRT | WO2017058771; SEQ ID NO: 150 | 10892 |
| MSP130 | VL | PRT | WO2017058771; SEQ ID NO: 158 | 10893 |
| MSP131 | VL | PRT | WO2017058771; SEQ ID NO: 168 | 10894 |
| MSP132 | VL | PRT | WO2017058771; SEQ ID NO: 174 | 10895 |
| MSP133 | VL | PRT | WO2017058771; SEQ ID NO: 182 | 10896 |
| MSP134 | VL | PRT | WO2017058771; SEQ ID NO: 190 | 10897 |
| MSP135 | VL | PRT | WO2017058771; SEQ ID NO: 198 | 10898 |
| MSP136 | VL | PRT | WO2017058771; SEQ ID NO: 296 | 10899 |
| MSP137 | VL | PRT | WO2017058771; SEQ ID NO: 222 | 10900 |
| MSP138 | VL | PRT | WO2017058771; SEQ ID NO: 230 | 10901 |
| MSP139 | VL | PRT | WO2017058771; SEQ ID NO: 238 | 10902 |
| MSP140 | VL | PRT | WO2017058771; SEQ ID NO: 246 | 10903 |
| MSP141 | VL | PRT | WO2017058771; SEQ ID NO: 254 | 10904 |
| MSP142 | VL | PRT | WO2017058771; SEQ ID NO: 262 | 10905 |
| MSP143 | VL | PRT | WO2017058771; SEQ ID NO: 270 | 10906 |
| MSP144 | VL | PRT | WO2017058771; SEQ ID NO: 278 | 10907 |
| MSP145 | VL | PRT | WO2017058771; SEQ ID NO: 286 | 10908 |
| MSP146 | VL | PRT | WO2017058771; SEQ ID NO: 294 | 10909 |
| MSP147 | VL | PRT | WO2017058771; SEQ ID NO: 302 | 10910 |
| MSP148 | VL | PRT | WO2017058771; SEQ ID NO: 310 | 10911 |
| MSP149 | VL | PRT | WO2017058771; SEQ ID NO: 318 | 10912 |
| MSP150 | VL | PRT | WO2017058771; SEQ ID NO: 326 | 10913 |
| MSP151 | VL | PRT | WO2017058771; SEQ ID NO: 334 | 10914 |
| MSP152 | VL | PRT | WO2017058771; SEQ ID NO: 337 | 10915 |
| MSP153 | VL | PRT | WO2017058771; SEQ ID NO: 242 | 10916 |

In some embodiments, the payload region of the AAV particle comprises a nucleic acid sequence encoding a polypeptide which is an antibody, an antibody-based composition, or a fragment thereof. As anon-limiting example, the antibody may be one or more of the polypeptides listed in Table 16, or variants or fragments thereof. As another non-limiting example, the antibody may be one or more of the heavy chain sequences listed in Table 16. As anon-limiting example, the antibody may be one or more of the light chain sequences listed in Table 16, or variants or fragments thereof.

In some embodiments, the payload region of the AAV particle comprises a nucleic acid sequence encoding a polypeptide comprising a heavy chain and alight chain sequence listed in Table 16, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and a light chain sequence listed in Table 16, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 16, one or more linkers from Table 2 and a heavy chain sequence from Table 16.

In some embodiments, the payload region comprises, in the 5′ to 3′ direction, an antibody heavy chain sequence, a linker region (may comprise one or more linkers) and alight chain sequence. In another embodiment, the linker is not used.

In some embodiments, the payload region comprises a nucleic acid sequence encoding, in the 5′ to 3′ direction, an antibody heavy chain sequence from Table 16, one or more linkers from Table 2, and alight chain sequence from Table 16.

In some embodiments, the payload region comprises a nucleic acid sequence encoding a single heavy chain. As a non-limiting example, the heavy chain is an amino acid sequence or fragment thereof described in Table 16.

Shown in Table 16 are a listing of antibodies and their polynucleotides and/or polypeptides sequences. These sequences may be encoded by or included in the AAV particles of the present disclosure. Variants or fragments of the antibody sequences described in Table 16 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 16. CDRs of the antibodies encoded by the viral genomes of the present disclosure may be 50%, 60%, 70%, 80%, 90%, 95% identical to CDRs listed in or incorporated in the sequences of Table 16. Methods of determining CDRs are well known in the art and are described herein. Payload regions may encode antibody variants with one or more heavy chain variable domain (V_{L}) or light chain variable domain (V₄) derived from the antibody sequences in Table 16. In some cases, such variants may include bispecific antibodies. Bispecific antibodies encoded by payload regions may comprise variable domain pairs from two different antibodies.

In some embodiments, the AAV particles may comprise a heavy and alight chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any multiple specific diseases and/or target-associated antibodies, not limited to those described in Table 16, including antibodies that are known in the art and/or antibodies that are commercially available. This may include fragments of such antibodies or antibodies that have been developed to comprise one or more of such fragments [e.g., variable domains or complementarity determining regions (CDRs)].

In some embodiments, the AAV particles may have a payload region comprising any of the multiple specific disease and/or target-associated antibodies as described in International Publication Number WO2019096797, WO2019086395, WO2019060713, WO2018224951, WO2018218076, WO2018215427, WO2018209055, WO2018199337, WO201815719, WO2018151375, WO2018144784, WO2018141894, WO2018129713, WO2018112253, WO2018106842, WO2018090052, WO2018075692, WO2018038469, WO2018037092, WO2017218707, WO2017210149, WO2017190079, WO2017184831, WO2017134667, WO2017121843, WO2017101828, WO2017097706, WO2017055539, WO2017055537, and WO2017049004, the contents of each of which are herein incorporated by reference in their entirety.

In some embodiments, viral genomes of the AAV particles of the present disclosure comprising nucleic acids which have been engineered to enable expression of antibodies as described herein for the treatment of a specific disease, disorder or condition may also be used for the treatment of any other disease, disorder or condition. Further, viral genomes may comprise any combination of nucleic acid sequences from any of Tables 3-16 or encode a combination of amino acid sequences described in any of Tables 3-16.

In certain embodiments, viral genomes of the AAV particles of the present disclosure may comprise nucleic acids which have been engineered to enable expression of clinically relevant antibodies including, but not limited to, antibodies recently approved or currently under review for use in a clinical trial, or antibodies in use in a Phase of a clinical trial, e.g. Phase 2/3 or Phase 3 of a clinical trial. In such embodiments, viral genomes of the AAV particles of the present disclosure may comprise nucleic acids which have been engineered to enable expression of clinically relevant antibodies including, but not limited to, Dupilumab (Dupixent), Durvalumab (Imfinzi), Emicizumab (Hemlibra), Guselkumab (Tremfya), Inotuzumab ozogamicin (Besponsa), Ocrelizumab (Ocrevus), Sarilumab (Kevzara), Avelumab (Bavencio), Brodalumab (Siliq, Lumicef, Kyntheum), Benralizumab (Fasenra), Mogamulizumab (Poteligeo), Burosumab, Erenumab (Aimovig), Fremanezumab, Galcanezumab (Emgality), Ibalizumab, Tildrakizumab, Caplacizumab (ALX-0081), Romosozumab (Evenity), Tislelizumab (BGB-A317), (vic-)trastuzumab duocarmazine, Aducanumab (BIIB037), Andecaliximab, Anifrolumab, BGB-A317, Brolucizumab, Carotuximab (TRC-105), Crenezumab, Cemiplimab (REGN2810), Enfortumab vedotin, Epratuzumab (LymphoCide), Eptinezumab (ALD403), Etrolizumab, Fasinumab, futuximab, Ganitumab, Gantenerumab, I-131-BC8 (lomab-B), Sintilimab (IBI308), Isatuximab, JS001, L191L2/L19TNF, Lanadelumab, Margetuximab, Mirvetuximab soravtansine, Moxetumomab pasudotox, Nimotuzumab, Olokizumab, OMS721, Onartuzumab (MetMAb), Oportuzumab monatox, Spartalizumab (PDR001), Polatuzumab vedotin, Rabimabs, Racotumomab, Ravulizumab (ALXN1210), Risankizumab, Rovalpituzumab tesirine, Sacituzumab govitecan, Satralizumab, SCT400, SGM-101, SHP-647, Tanezumab, Teprotumumab, Tezepelumab, Tralokinumab, Tremelimumab, Ublituximab, Utomilumab, RC18, Bermekimab (MABp1, Xilonix), DRL_TZ, Actoxumab (MK-3415), Modotuximab (as a component of Sym004), Trastuzumab deruxtecan (DS-8201), Phase 2/3, Teplizumab, BCD-085, BCD-100, Camrelizumab (SHR-1210), Emapalumab, Inebilizumab (MEDI-551), PRO-140 (PA14, Roledumab, XMAB-5574 (MOR208), Depatuxizumab mafodotin (ABT-414), and Crizanlizumab.

### Payload Antibodies: Additional Antibodies

In some embodiments, the payload region of the viral particle comprises one or more nucleic acid sequences encoding antibodies, variants or fragments thereof.

In some embodiments, the payload region of the viral particle comprises one or more nucleic acid sequences encoding one or more of the payload antibody polypeptides listed in any of International Publications, WO2017191559, WO2017191561 or WO2017191560 all to Prothena Biosciences, Limited, the contents of each of which are incorporated by reference herein in their entirety.

In some embodiments, the payload region of the viral particle comprises one or more nucleic acid sequences encoding one or more of the payload antibody polypeptides listed in Tables 3-53 of U.S. provisional patent application 62/844,433, the contents of which are herein incorporated by reference in their entirety, or variants or fragments thereof. As used herein, “antibody polynucleotide” refers to a nucleic acid sequence encoding an antibody polypeptide.

In some embodiments, the payload region of the viral particle may be any one or more of those described in U.S. provisional patent application 62/844,433, the contents of which are herein incorporated by reference in their entirety.

In some embodiments, a payload region of the present disclosure may comprise an antibody sequence of any antibody known in the art, described herein or in U.S. provisional patent application 62/844,433, the contents of which are herein incorporated by reference in their entirety, or a fragment, variant or derivative thereof. In some embodiments, the payload may be a bispecific antibody.

In some embodiments, payloads of the present disclosure may comprise an Fc swap component, wherein said Fc swap may mediate direct cell killing.

### Systemic Disease Antibodies

In some embodiments, the payload region of the viral particle comprises one or more nucleic acid sequences encoding one or more of the systemic disease payload antibody polypeptides listed in Table 12 of U.S. provisional patent application 62/844,433 (SYS1-SYS73; SEQ ID NO: 20906-2097), the contents of which are herein incorporated by reference in their entirety.

### Foodborne Illness and Gastroenteritis Related Antibodies

In some embodiments, the payload region of the viral particle comprises one or more nucleic acid sequences encoding one or more of the gastrointestinal and food illness related payload antibody polypeptides listed in Tables 14-20 of U.S. provisional patent application 62/844,433, the contents of which are herein incorporated by reference in their entirety.

In some embodiments, the payload region of the viral particle comprises one or more nucleic acid sequences encoding one or more of the payload antibody polypeptides listed in Table 14 of U.S. provisional patent application 62/844,433 against Clostridium Difficile toxins (CD1-CD141; SEQ ID NO: 22301-22441), the contents of which are herein incorporated by reference in their entirety.

In some embodiments, the payload region of the viral particle comprises one or more nucleic acid sequences encoding one or more of the payload antibody polypeptides listed in Table 15 of U.S. provisional patent application 62/844,433, against Campylobacter jejuni (CAMP1-CAMP10; SEQ ID NO: 22442-22451), the contents of which are herein incorporated by reference in their entirety.

In some embodiments, the payload region of the viral particle comprises one or more nucleic acid sequences encoding one or more of the payload antibody polypeptides listed in Table 16 of U.S. provisional patent application 62/844,433 against bacterial infections of the intestine (BACG1-BACG98; SEQ ID NO: 22452-22549), the contents of which are herein incorporated by reference in their entirety.

In some embodiments, the payload region of the viral particle comprises one or more nucleic acid sequences encoding one or more of the payload antibody polypeptides listed in Table 17 of U.S. provisional patent application 62/844,433 against Hepatitis A and/or Hepatitis E (HEPAE1-HEPAE41; SEQ ID NO: 22550-22590), the contents of which are herein incorporated by reference in their entirety.

In some embodiments, the payload region of the viral particle comprises one or more nucleic acid sequences, fragment or variants thereof or encodes one or more polypeptides, fragments or variants thereof described in Chinese Pub. No. CN103923881, CN103923882, CN1605628, CN1318565, CN1163512, the contents of each of which are herein incorporated by reference in their entirety, against HAV.

In some embodiments, the payload region of the viral particle comprises one or more nucleic acid sequences encoding one or more of the payload antibody polypeptides listed in Table 18 of U.S. provisional patent application 62/844,433 against Norwalk virus (NORV1-NORV48; SEQ ID NO: 22591-22638), the contents of which are herein incorporated by reference in their entirety.

In some embodiments, the payload region of the viral particle comprises one or more nucleic acid sequences encoding one or more of the payload antibody polypeptides listed in Table 19 of U.S. provisional patent application 62/844,433 against Rotavirus (ROTV1-ROTV25; SEQ ID NO: 22639-22663), the contents of which are herein incorporated by reference in their entirety.

In some embodiments, the payload region of the viral particle comprises one or more nucleic acid sequences encoding one or more of the payload antibody polypeptides listed in Table 20 of U.S. provisional patent application 62/844,433 against Entamoeba Histolytica (ENTH1-ENTH16; SEQ ID NO: 22664-22679), the contents of which are herein incorporated by reference in their entirety.

In some embodiments, the payload region of the viral particle comprises one or more nucleic acid sequences encoding one or more of the payload antibody polypeptides, fragments or variants thereof described in International Pub. No. WO2001012646, the contents of which are herein incorporated by reference in their entirety, against Listeria monocytogenes, salmonella and/or leishmania.

### Neglected Tropical Diseases

In some embodiments, the payload region of the viral particle comprises one or more nucleic acid sequences encoding one or more of the neglected tropical disease related payload antibody polypeptides listed in Tables 21-24 of U.S. provisional patent application 62/844,433, the contents of which are herein incorporated by reference in their entirety.

In some embodiments, the payload region of the viral particle comprises one or more nucleic acid sequences encoding one or more of the payload antibody polypeptides listed in Table 21 of US provisional patent application 62/844,433 against Dengue Fever Virus (DENG1-DENG123; SEQ ID NO: 22680-22802), the contents of which are herein incorporated by reference in their entirety.

In some embodiments, the payload region of the viral particle comprises one or more nucleic acid sequences encoding one or more of the payload antibody polypeptides, fragments or variants thereof described in International Pub. No. WO2013089647 and WO2013035345, U.S. Pat. No. 8,637,035 and US887187, US Publication No. US20050123900, and Chinese Patent Publication No. CN102757480, the contents of which are herein incorporated by reference in their entirety, against Listeria monocytogenes, salmonella and/or leishmania.

In some embodiments, the payload region of the viral particle comprises one or more nucleic acid sequences encoding one or more of the payload antibody polypeptides listed in Table 22 of U.S. provisional patent application 62/844,433 against Rabies Virus (RABV1-RABV91; SEQ ID NO: 22803-22893), the contents of which are herein incorporated by reference in their entirety.

In some embodiments, the payload region of the viral particle comprises one or more nucleic acid sequences encoding one or more of the payload antibody polypeptides listed in Table 23 of U.S. provisional patent application 62/844,433 against Chagas Virus (CHAG1-CHAG2; SEQ ID NO: 22894-22895), the contents of which are herein incorporated by reference in their entirety.

In some embodiments, the payload region of the viral particle comprises one or more nucleic acid sequences encoding one or more of the payload antibody polypeptides listed in Table 24 of U.S. provisional patent application 62/844,433 against Chikungunya Virus (CHIK1-CHIK6; SEQ ID NO: 22896-22901), the contents of which are herein incorporated by reference in their entirety.

In some embodiments, the payload region of the viral particle comprises one or more nucleic acid sequences, fragment or variants thereof or encodes one or more polypeptides, fragments or variants thereof encoding antibodies described International Pub No. WO1983001785 and U.S. Pat. No. 5,827,671, the contents of each of which are herein incorporated by reference in their entirety, against the protozoan parasite Leishmania.

In some embodiments, the payload region of the viral particle comprises one or more nucleic acid sequences, fragment or variants thereof or encodes one or more polypeptides, fragments or variants thereof encoding antibodies against the Buruli ulcer (Mycobacterium ulcerans), Leprosy/Hansen's disease (Mycobacterium leprae), Leishmaniasis, Cysticercosis, Dracunculiasis (Guinea Worm Disease), Echinococcosis, Fascioliasis, Human African Trypanosomiasis (African Sleeping Sickness), Lymphatic filariasis, Onchocerciasis, Schistosomiasis, Soil-transmitted Helminths (STH).

### Toxins

In some embodiments, the payload region of the viral particle comprises one or more nucleic acid sequences encoding one or more of the toxin related payload antibody polypeptides listed in Tables 25-28 of U.S. provisional patent application 62/844,433, the contents of which are herein incorporated by reference in their entirety.

In some embodiments, the payload region of the viral particle comprises one or more nucleic acid sequences encoding one or more of the payload antibody polypeptides listed in Table 25 of US provisional patent application 62/844,433 against Ricin Toxin (RICN1-RICN20; SEQ ID NO: 22902-22921), the contents of which are herein incorporated by reference in their entirety.

In some embodiments, the payload region of the viral particle comprises one or more nucleic acid sequences encoding one or more of the payload antibody polypeptides listed in Table 26 of U.S. provisional patent application 62/844,433 against Anthrax (ANTH1-ANT H245; SEQ ID NO: 22922-23166), the contents of which are herein incorporated by reference in their entirety.

In some embodiments, the payload region of the viral particle comprises one or more nucleic acid sequences encoding one or more of the payload antibody polypeptides listed in Table 27 of U.S. provisional patent application 62/844,433 against Botulinum Toxin (BOTT1-BOTT30; SEQ ID NO: 23167-23196), the contents of which are herein incorporated by reference in their entirety.

In some embodiments, the payload region of the viral particle comprises one or more nucleic acid sequences encoding one or more of the payload antibody polypeptides listed in Table 28 of U.S. provisional patent application 62/844,433 against Shiga Toxin (SHIG1-SHIG71; SEQ ID NO: 23197-23267), the contents of which are herein incorporated by reference in their entirety.

In some embodiments, the payload region of the viral particle comprises one or more nucleic acid sequences, fragments or variants thereof or encodes one or more polypeptides, fragments or variants thereof described in US Pub. No. US20090280104, the contents of each of which are herein incorporated by reference in their entirety, against Shiga toxin.

### Tropical Diseases

In some embodiments, the payload region of the viral particle comprises one or more nucleic acid sequences encoding one or more of the tropical disease related payload antibody polypeptides listed in Tables 29-31 of U.S. provisional patent application 62/844,433, the contents of which are herein incorporated by reference in their entirety.

In some embodiments, the payload region of the viral particle comprises one or more nucleic acid sequences encoding one or more of the payload antibody polypeptides listed in Table 29 of U.S. provisional patent application 62/844,433 against Plasmodium Falciparum causing Malaria (MALA1-MALA57; SEQ ID NO: 23268-23324), the contents of which are herein incorporated by reference in their entirety.

In some embodiments, the payload region of the viral particle comprises one or more nucleic acid sequences encoding one or more of the payload antibody polypeptides listed in Table 30 of U.S. provisional patent application 62/844,433 against Ebola and/or Margburg Viruses (EBOL1-EBOL53; SEQ ID NO: 23325-23377), the contents of which are herein incorporated by reference in their entirety.

In some embodiments, the payload region of the viral particle comprises one or more nucleic acid sequences, fragment or variants thereof or encodes one or more polypeptides, fragments or variants thereof described in U.S. Pat. No. 7,335,356 and EP Pub. No. EP1539238, the contents of each of which are herein incorporated by reference in their entirety, against Ebola.

In some embodiments, the payload region of the viral particle comprises one or more nucleic acid sequences encoding one or more of the payload antibody polypeptides listed in Table 31 of U.S. provisional patent application 62/844,433 against Mosquito-borne disease (MOSQ1-MOSQ118; SEQ ID NO: 23378-23495), the contents of which are herein incorporated by reference in their entirety.

In some embodiments, the payload region of the viral particle comprises one or more nucleic acid sequences, fragment or variants thereof or encodes one or more polypeptides, fragments or variants thereof described in U.S. Pat. No. 6,399,062 and US Pub. No. US20110171225, the contents of each of which are herein incorporated by reference in their entirety, against Malaria.

### Disease Specific Epitopes, Innate Defense Regulator Peptides, Cyclic Peptides

In some embodiments, the viral genomes of the AAV particles may comprise nucleic acids which have been engineered to enable expression of antibodies binding to disease-specific epitopes of proteins. Such antibodies may be used to diagnose, prevent, and/or treat the corresponding medical conditions by targeting epitopes of the protein presented by or accessible on native or non-native forms (e.g., misfolded forms of native proteins) of the target. Such epitopes may be specific to diseases involved with misfolding of a protein due to pathologic condition and resulting in misfolded aggregates. The disease-specific proteins are considered to be toxic to neurons and to have a role in neuronal cell death and dysfunction in neurodegenerative diseases including, but not limited to, Alzheimer's disease (AD), amyotrophic lateral sclerosis (ALS), Parkinson's disease, dementia by Lewy body (DLB), and prion diseases, e.g. Creutzfeldt-Jakob disease (CJD), Gerstmann. Straussler-Scheinker syndrome (GSS), kuru, and fatal familial insomnia (FFI).

In some embodiments, the encoded disease-specific epitopes may include epitopes on SOD1 that are revealed as SOD1 (Superoxide dismutase [Cu—Zn]) dissociates from its homodimeric, normal state. The SOD epitopes may be selectively presented or accessible in non-native SOD1 forms including misfolded SOD1 monomer, misfolded SOD1 dimer, and the epitopes selectively presented or accessible in SOD1 aggregates. Such epitopes may be specific to neurodegenerative diseases including, but not limited to, amyotrophic lateral sclerosis (ALS), Alzheimer's (AD), Parkinson's (PD), and Lewy body diseases (LBD).

In some embodiments, the expressed antibodies may bind to epitopes presented by or accessible on non-native forms of SOD1, such as those presented by SED ID NO: 2, 3, 5, 6, and 7 of U.S. Pat. No. 7,977,314 (the contents of which are herein incorporated by reference in its entirety), or presented by or accessible on monomeric forms of SOD1, such as those presented by SEQ ID NOs: 1 and 4 of U.S. Pat. No. 7,977,314, the contents of which are herein incorporated by reference in their entirety. In some embodiments, the expressed antibodies may comprise isolated peptides corresponding to such epitopes, such as those presented in SEQ ID NOs: 18 or SEQ ID NOs: 8-16, or epitopes presented by SEQ ID NOs: 34-63, 65-79 of U.S. Pat. No. 7,977,314, the contents of which are herein incorporated by reference in their entirety.

In some embodiments, the encoded disease-specific epitopes may be specific to diseases associated with prion protein (PrP); familial amyloid polyneuropathy or senile systemic amyloidosis or a disease related by the presence of misfolded transthyretine (TTR); renal accumulation of 02 microglobulin amyloid deposits or a disease related by the presence of misfolded β2 microglobulin, amyotrophic lateral sclerosis (ALS) or a disease related by the presence of misfolded SOD1; leukemias or myelomas or a disease related by the presence of misfolded cluster of differentiation 38 (CD38); colon cancer metastasis and or a disease related by the presence of misfolded cluster of differentiation (CD44); tumors associated with tumor necrosis factor receptor (TNFR); cancers including cervical, head and neck, endometrial, lung and breast carcinomas, pleural mesotheliomas, malignant melanomas, Hodgkin lymphomas, anaplastic large cell non-Hodgkin lymphomas, or a disease related by the presence of misfolded Notch homolog 1 (NOTCH1) e.g. acute myeloid leukemias and B-cell chronic lymphoid leukemias; cancer in which Fas receptor (FasR) is implicated; cancers and related disorders in which misfolded epidermal growth factor (EGFR) is implicated; and/or other related diseases, disorders and conditions.

In some embodiments, the encoded disease specific epitopes may include epitopes that are revealed as the proteins misfold. In some embodiments, the expressed antibodies may bind to predicted epitopes of human PrP, such as those presented by SEQ ID NOs: 1-10 of US Patent Publication No. US20100233176; bovine PrP, such as those presented by SEQ ID NOs: 11-15 of US Patent Publication No. US20100233176, TTR, such as those presented by SEQ ID NOs: 1622 of US Patent Publication No. US20100233176; beta-2 microglobulin, such as those presented by SEQ ID NOs: 23-26 of US Patent Publication No. US20100233176; SOD1, such as those presented by SEQ ID NOs: 27-40 of US Patent Publication No. US20100233176; CD38, such as those presented by SEQ ID NOs: 41-45 of US Patent Publication No. US20100233176; CD44, such as those presented by 46-50 of US Patent Publication No. US20100233176; TNFR, such as those presented by 51-55 of US Patent Publication No. US20100233176; notch protein, such as those presented in SEQ ID NOs: 56-60 of US Patent Publication No. US20100233176; FasR, such as those presented by SEQ ID NOs: 61-65 of US Patent Publication No. US20100233176 and EGFR, such as those presented by SEQ ID NOs: 66-80 of US Patent Publication No. US20100233176; the contents of which are herein incorporated by reference in their entirety.

In some embodiments, the expressed antibodies may comprise peptides corresponding to such epitopes. In some embodiments, the expressed antibodies may comprise prion-specific peptides, such as those presented by SEQ ID NOs: 81-88 of US Patent Publication No. US20100233176, the contents of which are herein incorporated by reference in their entirety, and variations thereof.

In some embodiments, the encoded disease-specific epitopes may be specific to prion diseases, including transmissible spongiform encephalopathies (TSEs) or other prion diseases. In some embodiments, the expressed antibodies may bind to predicted epitopes of PrP, such as those presented by SEQ ID NOs: 24, 26, 28, 30, 32, 34, 36, 39.43, of US Patent Publication No. US20150004185, the contents of which are herein incorporated by reference in their entirety. In some embodiments, the expressed antibodies may comprise prion-specific peptides or peptide fusions, such as those presented by SEQ ID NOs: 12-23, 25, 27, 29, 31, 33, 35, 37, 38, 43, and 44-48 of US Patent Publication No. US20150004185, the contents of which are herein incorporated by reference in their entirety.

In some embodiments, the expressed antibodies may comprise prion peptides binding to prion specific abnormal isoform of the prion protein, such as those presented by SEQ ID NOs: 2-10 of US Patent Publication No. US20040072236, the contents of which are herein incorporated by reference in their entirety.

In some embodiments, the viral genomes of the AAV particles may comprise nucleic acids which have been engineered to express innate defense regulator (IDR) peptides. IDRs are immunomodulatory peptides that act directly on cells to effect an innate immune response. Such IDRs may be used to treat neurodegenerative diseases associated with neuroinflammation, e.g. amyotrophic lateral sclerosis (ALS), Alzheimer's disease, Friedreich's ataxia, Huntington's disease, Lewy body disease, Parkinson's disease, spinal muscular atrophy, and multiple sclerosis (MS) and other neurodegenerative diseases. In some embodiments, IDRs may be those presented by SEQ ID NOS: 1-969, and 973-1264 of International Publication No. WO2013034982, the contents of which are herein incorporated by reference in their entirety, or analogs, derivatives, amidated variations and conservative variations thereof.

In some embodiments, the viral genomes of the AAV particles may comprise nucleic acids which have been engineered to express antibodies binding to an epitope of the Tropomyosin receptor kinase (TrkC) receptor. Such antibodies may comprise a peptide, such as one presented by SEQ ID NO: 1 of U.S. Pat. No. 9,200,080, the contents of which are herein incorporated by reference in their entirety.

In some embodiments, the viral genomes of the AAV particles may comprise nucleic acids which have been engineered to express cyclic peptides with an amino acid sequence SNK. Non-limiting examples of other cyclic peptides include SEQ ID NO:1.7 of U.S. Pat. No. 9,216,217, the contents of which are herein incorporated by reference in their entirety. The method of preparing the antibodies may include hyperimmune preparation method, as described in U.S. Pat. No. 9,216,217, the contents of which are herein incorporated by reference in their entirety.

### Prions

In some embodiments, the viral genomes of the AAV particles may comprise a nucleic acid sequence encoding antibodies comprising prion peptides comprising prion epitopes, and fusions and repeats thereof, such as those presented by SEQ ID NOs: 8-32, 35, and 36 of U.S. Pat. No. 9,056,918, the contents of which are herein incorporated by reference in their entirety.

In some embodiments, the viral genomes of the AAV particles may comprise a nucleic acid sequence encoding prion binding proteins (PrPBP). In some embodiments, the PrPBPs are cadherins, such as those presented by SEQ ID NOs: 1 and 2 of International Publication WO1997/045746, the contents of which are herein incorporated by reference in their entirety. In some embodiments, the PrPBPs are cadherins, such as those presented by SEQ ID NOs: 2 and 7-9 of International Publication No. WO2001000235, the contents of which are herein incorporated by reference in their entirety.

### The Nature of the Polypeptides and Variants

Antibodies encoded by payload regions of the viral genomes may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phospho-threonine and/or phospho-serine. Alternatively, variant mimics may result in deactivation or in an inactivated product containing the mimic, e.g., phenylalanine may act as an inactivating substitution for tyrosine; or alanine may act as an inactivating substitution for serine.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80%, more preferably at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions, or deletions of amino acid residues that still maintain the properties of the parent polypeptide.

Sequence tags or amino acids, such as one or more lysines, can be added to the peptide sequences (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure include surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7-amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7-amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid-based embodiments is used synonymous with “amino acid residue” and “amino acid side chain”. A site represents a position within a peptide or polypeptide that may be modified, manipulated, altered, derivatized or varied within the polypeptide-based molecules of the present disclosure.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide-based molecules of the present disclosure may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a component of a molecule, any of several manipulations and/or modifications of these features may be performed by moving, swapping, inverting, deleting, randomizing, or duplicating. Furthermore, it is understood that manipulation of features may result in the same outcome as a modification to the molecules. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Payload Regulation

In one embodiment, expression of payloads from viral genomes may be regulated using various methods known in the art. In such embodiments, a type of regulation may be e.g. temporal or spatial. In other such embodiments, a type of regulation may be e.g. temporary or permanent. In certain embodiments, expression of payloads from viral genomes may be upregulated to increase, enhance or accelerate the rate of expression. In certain embodiments, expression of payloads from viral genomes may be downregulated to decrease, halt, or decelerate the rate of expression.

While not wishing to be bound by theory, destabilizing domains are known to confer instability and decrease transgene expression. The presence of the destabilizing domain can trigger the cell's proteasomal degradation systems, which then can lead to antibody destruction. Destabilizing domains may comprise peptide sequences which are rich in a particular subset of amino acids which are thought to signal degradation, such as, but not limited to, proline, glutamic acid, serine and threonine (known as PEST sequences).

In some embodiments, the expression of the payload may be regulated by fusion with a stabilizing or a destabilizing domain. Stabilizing and destabilizing domains which can be used are well known in the art. Non-limiting examples of destabilizing domains include FK506 Binding Protein (FKBP), E. coli dihydrofolate reductase (DHFR), mouse ornithine decarboxylase (MODC), or estrogen receptors (ER). Examples of destabilizing domains, their ligands and/or binding partners are taught in International Publication WO2017180587 (Application: PCTUS2017026950), the contents of which are incorporated herein by reference in their entirety.

In some embodiments the destabilizing domain may be inducible. In some embodiments the destabilizing domain may be a “single ligand-single domain,” which allows control of protein stability through a small molecule ligand. In some embodiments the destabilizing domain may be FK506- and rapamycin-binding protein (FKBP12) destabilizing domain, which can be regulated by rapamycin and its analogs, and is unstable in the absence of its ligand. In one embodiment, a point mutant (L106P) of the 107-amino acid protein FKBP confers instability to fusion partners, and this instability is reversed by a synthetic ligand named Shield-1, as described in Banaszynski, L, Chen, L., Maynard-Smith, L. A., Ooi, G. L. and Wandless, T. J. A rapid, reversible, and tunable method to regulate protein function in living cells using synthetic small molecules. Cell 126, 995-1004 (2006), the contents of which is herein incorporated by reference in its entirety.

In another embodiment, the destabilizing domain may be derived from E. Coli dihydrofolate reductase. In some embodiments the small molecule trimethoprim (TMP) can bind to the domain and act as a stabilizer, for example, as described in Iwamoto et al. (Chem Biol. 2010 September 24; 17(9):981-8. A general chemical method to regulate protein stability in the mammalian central nervous system) the contents of which is herein incorporated by reference in its entirety. This system has been shown to be applied to regulation of glia cell derived neurotrophic factor (GDNF), as described in Tai et al. (DOI: 10.1371/journal.pone.0046269, Destabilizing Domains Mediate Reversible Transgene Expression in the Brain), the contents of which is herein incorporated by reference in its entirety.

In another embodiment, the destabilizing domain may be alight sensitive degradation domain. In a non-limiting example, the light sensitive degradation domain may be one of the domains described in U.S. Pat. No. 9,115,184, the contents of which is herein incorporated by reference in its entirety. In one embodiment, the domain comprises LOV24.

It is contemplated as part of the disclosure that any of the destabilizing domains may be combined with any of the payloads, e.g., antibodies or fragments thereof, and/or other proteins or fusion proteins described herein.

In one embodiment, an antibody payload may be fused to a destabilizing domain. In one embodiment, an antibody payload may be fused to a destabilizing domain which can be further regulated by a ligand.

In some embodiments, nucleic acid sequences from any of Tables 3-16 may comprise a CDS or coding region that enables expression of amino acid sequences such as those presented in SEQ ID NO:10917 to SEQ ID NO: 10927.

### Antibody Regulation

In some embodiments, the quantity, i.e., level or amount, or activity, e.g. binding affinity, of an antibody, or fragment or variant thereof, of the present disclosure may be regulated. As a non-limiting example, the quantity or activity of the antibody may be upregulated, enhanced, or increased. As another non-liming example, the quantity or activity of an antibody may be downregulated, suppressed or reduced. In certain embodiments, the regulated antibody may be an endogenous antibody. As a non-limiting example, the endogenous antibody may be an anti-AAV neutralizing antibody (NAb). In certain embodiments, the regulated antibody may be any antibody, or fragment or variant thereof, encoded by payload regions of the viral genomes of the present disclosure. The regulation of the antibody may be therapeutically effective, prophylactically effective, or diagnostically effective.

In some embodiments, the quantity or activity of an antibody may be regulated by a protease. In some embodiments, the protease may be encoded by payload regions of the viral genomes of the AVV particles of the present disclosure. In some embodiments, the protease may be prepared as a pharmaceutical composition such as, but not limited to, a pharmaceutical composition comprising a protease diluted in an infusion solution.

In some embodiments, the protease encoded by payload regions of the viral genomes of the present disclosure, also termed the protease AAV particle herein, may be administered or delivered alone, i.e. separately, or in combination, with the protease pharmaceutical composition. In some embodiments, the protease pharmaceutical composition may be administered or delivered alone, i.e. separately, or in combination, with the protease AAV particle. In some embodiments the protease AAV particle and/or the protease pharmaceutical composition may be administered or delivered alone, i.e. separately, or in combination with any AAV of the present disclosure.

In some embodiments, the protease AAV particle, may be administered or delivered in combination with the protease pharmaceutical composition at the same time, i.e. simultaneously, or independently, at different times. In some embodiments, the protease AAV particle and/or the protease pharmaceutical composition may be administered in combination with any AAV of the present disclosure at the same time, i.e. simultaneously, or independently, at different times.

In some embodiments the protease AAV particle and/or the protease pharmaceutical composition may be administered in a single dose. In some embodiments the protease AAV particle and/or the protease pharmaceutical composition may be administered in multiple doses.

In some embodiments, the protease AAV particles or protease pharmaceutical compositions may be administered by any delivery route described herein. As a non-limiting example, the protease AAV particles or protease pharmaceutical compositions may be administered via an intravenous delivery route.

In some embodiments, the quantity or activity of an antibody may be regulated by a protease that may be a streptococcal protease. As a non-limiting example, the streptococcal protease is IdeS (Immunoglobulin G-degrading enzyme of streptococcus pyrogenes), as described by Winstedt et al, 2015 (see Winstedt et al, PLoS One. 2015 Jul. 15; 10(7):e0132011) and by Järnum et al, 2019 (see, J Immunol. 2015 Dec. 15; 195(12):5592-601), the contents of each of which are herein incorporated by reference in their entirety.

In some embodiments, IdeS cleaves immunoglobulin G (IgG) antibodies. In some embodiments, IdeS cleaves an IgG antibody first by generating a single-cleaved IgG molecule (scIgG) with one intact heavy chain, and then by generating one F(ab′)2 fragment and one homo-dimeric Fc fragment, which are held together by non-covalent interactions. In some embodiments, IdeS cleavage of immunoglobulin G (IgG) antibodies leads to their enzymatic inactivation.

In some embodiments, IdeS cleaves the entire IgG pool, resulting in complete removal of IgG antibodies. In some embodiments, IdeS cleaves a portion of the IgG pool, resulting in partial removal of IgG antibodies.

In some embodiments, IdeS removal or reduction of the IgG pool may be temporary, or impermanent. In such embodiments, the IgG pool may be restored following IdeS administration or delivery to levels consistent with those prior to IdeS administration or delivery. In some embodiments, newly synthesized IgG may be detectable one to two weeks following IdeS administration or delivery, and may constitute the main IgG fraction by three weeks following IdeS administration or delivery. In some embodiments, antibiotics may be given until the IgG pool is restored following IdeS administration or delivery to levels consistent with those prior to IdeS administration or delivery.

In some embodiments, further proteolytic cleavage by IdeS may be prevented with a protease inhibitor. As a non-limiting example, the protease inhibitor is iodoacetic acid.

In some embodiments, IdeS may cleave an antigen-specific IgG antibody. The antigen-specific IgG antibody may be endogenous, or exogenous. As a non-limiting example, the endogenous antigen-specific IgG antibody may be an anti-AAV neutralizing antibody (NAb), which, when cleaved, may be associated with enhanced AAV transduction efficacy. As a non-limiting example, the exogenous antigen-specific IgG antibody may be any antibody, or fragment or variant thereof, encoded by payload regions of the viral genomes of the present disclosure.

In some embodiments, IdeS may cleave an IgG antibody to clear, or unblock, effector cell antibody receptors, as described by Järnum et al, 2019 (see. J Immunol. 2015 Dec. 15; 195(12):5592-601), the contents of which are herein incorporated by reference in their entirety. In such embodiments, the clearance or unblocking of effector cell receptors may allow for preferential loading (binding) of other, i.e., non-IdeS-cleaved antibodies, onto effector cells, thereby enhancing or potentiating their binding and/or efficacy. As a non-limiting example, antibodies encoded by payload regions of the viral genomes of the present disclosure, may preferentially load onto effector cells following IdeS cleavage of IgG antibodies, thereby enhancing or potentiating their binding and/or efficacy.

In some embodiments, IdeS cleavage and processing efficacy may be investigated using different methods. As a non-limiting example, IdeS cleavage and processing efficacy may be investigated using an ELISA that measures IgG and IgG fragment levels in serum. As another non-limiting example, IdeS cleavage and processing efficacy may be investigated using an ELISA that may measure the dynamics of the F(ab′)2- and Fc-containing fragments.

### AAV Production

The present disclosure provides methods for the generation of parvoviral particles, e.g. AAV particles, by viral genome replication in a viral replication cell.

In accordance with the disclosure, the viral genome comprising a payload region encoding an antibody, an antibody-based composition or fragment thereof, will be incorporated into the AAV particle produced in the viral replication cell. Methods of making AAV particles are well known in the art and are described in e.g., States U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, and WO2001023597; Methods In Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822.8 (1989); Kajigaya et al., Proc. Natl. Acad. Sci. USA 88: 4646.50 (1991); Ruffing et al., J. Vir. 66:6922-30 (1992); Kimbauer et al., Vir, 219:37-44 (1996); Zhao et al., Vir.272:38293 (2000); the contents of each of which are herein incorporated by reference in their entirety. In some embodiments, the AAV particles are made using the methods described in WO2015191508, the contents of which are herein incorporated by reference in their entirety.

Viral replication cells commonly used for production of recombinant AAV viral vectors include but are not limited to 293 cells, COS cells, HeLa cells, KB cells, and other mammalian cell lines as described in U.S. Pat. Nos. U.S. Pat. Nos. 6,156,303, 5,387,484, 5,741,683, 5,691,176, and 5,688,676; U.S. patent publication No. 200210081721, and International Patent Publication Nos. WO 00/47757, WO 0024916, and WO 96/17947, the contents of each of which are herein incorporated by reference in their entireties.

In some embodiments, the AAV particles of the present disclosure may be produced in insect cells (e.g., Sf9 cells).

In some embodiments, the AAV particles of the present disclosure may be produced using triple transfection.

In some embodiments, the AAV particles of the present disclosure may be produced in mammalian cells.

In some embodiments, the AAV particles of the present disclosure may be produced by triple transfection in mammalian cells.

In some embodiments, the AAV particles of the present disclosure may be produced by triple transfection in HEK293 cells.

The present disclosure provides a method for producing an AAV particle comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, 5) harvesting and purifying the viral particle comprising a parvoviral genome.

In some embodiments, the present disclosure provides a method for producing an AAV particle comprising the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

In some embodiments, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In some embodiments, the viral construct vector(s) used for AAV production may contain a nucleotide sequence encoding the AAV rep proteins where the initiation codon of the AAV rep protein or proteins is a non-ATG. In some embodiments, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the viral genome of the AAV particle optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are selected from those described in International Application No. WO 96/23810; Heim et al., Current Biology 2:178-182 (1996); Heim et al., Proc. Natl. Acad. Sci. USA (1995); or Heim et al., Science 373:663-664 (1995); WO 96/30540, the contents of each of which are incorporated herein by reference in their entireties).

The AAV viral genomes encoding an anti-tau antibody payload described herein may be useful in the fields of human disease, veterinary applications and a variety of in vivo and in vitro settings. The AAV particles of the present disclosure may be useful in the field of medicine for the treatment, prophylaxis, palliation or amelioration of neurological diseases and/or disorders. In some embodiments, the AAV particles are used for the prevention and/or treatment of a tauopathy.

Various embodiments herein provide a pharmaceutical composition comprising the AAV particles described herein and a pharmaceutically acceptable excipient.

Various embodiments herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of: intravenous, intracerebroventricular, intraparenchymal, intrathecal, subpial and intramuscular, or a combination thereof. Certain embodiments of the method provide that the subject is treated for a tauopathy and/or other neurological disorder. In one aspect of the method, a pathological feature of the tauopathy or other neurological disorder is alleviated and/or the progression of the tauopathy or other neurological disorder is halted, slowed, ameliorated or reversed.

Various embodiments herein describe a method of decreasing the level of soluble tau in the central nervous system of a subject in need thereof comprising administering to said subject an effective amount of the pharmaceutical composition described herein.

Also described herein are compositions, methods, processes, kits and devices for the design, preparation, manufacture and/or formulation of AAV particles. In some embodiments, payloads, such as but not limited to anti-tau antibodies, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of neurological disease, such as, but not limited to tauopathy.

AAV Particles Comprising Viral Genomes with Antibody Payloads

In some embodiments, the AAV particle comprises a viral genome with a payload region comprising one or more antibody polynucleotide sequences. In such an embodiment, a viral genome encoding more than one polypeptide may be replicated and packaged into a viral particle. A target cell transduced with a viral particle comprising one or more antibody polynucleotides may express the encoded antibody or antibodies in a single cell.

In some embodiments, the AAV particles are useful in the field of medicine for the treatment, prophylaxis, palliation or amelioration of diseases and/or disorders.

In some embodiments, the AAV particles comprising antibody polynucleotide sequences which comprise a nucleic acid sequence encoding at least one antibody heavy and/or light chain may be introduced into mammalian cells.

The V viral genomes encoding antibody polynucleotides described herein may be useful in the fields of human disease, viruses, infections veterinary applications and a variety of in vivo and in vitro settings. In some embodiments, the AAV viral genomes encoding antibody polynucleotides are used for the prevention and/or treatment of a disease, disorder and/or condition.

The viral genome of the AAV particles of the present disclosure may comprise any combination of the sequence regions described in Tables 17-24 encapsidated in any of the capsids listed in Table 1 or described herein.

In some embodiments, the AAV particle viral genome may comprise at least one sequence region as described in Tables 17-24. The regions may be located before or after any of the other sequence regions described herein. Viral genomes may further comprise more than one copy of one or more sequence regions as described in Tables 17-24.

In some embodiments, the AAV particle viral genome may comprise at least one inverted terminal repeat (ITR) region. The ITR region(s) may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146,147, 148,149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR region for the viral genome may be 7580, 7585, 75-100, 80-85, 80-90, 80-105, 85-90, 85-95, 85-110, 90-95, 90-100, 90-115, 95-100, 95-105, 95-120, 100-105, 100-110,100-125, 105-110,105-115, 105-130, 110-115, 110-120, 110-135, 115-120, 115-125, 115-140, 120-125,120-130, 120.145, 125-130,125-135, 125-150,130-135, 130-140, 130-155, 135-140, 135-145,135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises a 5′ ITR that is about 141 nucleotides in length. As a non-limiting example, the viral genome comprises a 5′ ITR that is about 130 nucleotides in length.

In some embodiments, the AAV particle viral genome comprises at least one 5′ inverted terminal repeat (5′ ITR) sequence region. Non-limiting examples of 5′ ITR sequence regions are described in Table 17.

TABLE 17
Inverted Terminal Repeat (ITR) Sequence Regions

| Sequence | Sequence | |
|---|---|---|
| Region Name | Length | SEQ ID NO |
| ITR1 | 130 | 13519 |
| ITR2 | 141 | 13520 |
| ITR3 | 130 | 13521 |
| ITR4 | 141 | 13522 |

In some embodiments, the AAV particle viral genome may have an ITR that comprises ITR1. In some embodiments, the AAV particle viral genome may have an ITR that comprises ITR2. In some embodiments, the AAV particle viral genome may have an ITR that comprises ITR3. In some embodiments, the AAV particle viral genome may have an ITR that comprises ITR4.

In some embodiments, the AAV particle viral genome may have two ITRs. As a non-limiting example, the two ITRs are ITR1 and ITR3. As a non-limiting example, the two ITRs are ITR1 and ITR4. As a non-limiting example, the two ITRs are ITR2 and ITR3. As a non-limiting example, the two ITRs are ITR2 and ITR4

In some embodiments, the AAV particle viral genome may comprise at least one promoter sequence region. The promoter sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181,182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 and more than 600 nucleotides. The length of the promoter region for the viral genome may be 4-10, 10-20, 10-50, 20-30, 30-40, 40-50, 50-60, 50-100, 60-70, 70-80, 80-90, 90-100,100-110, 100-150, 110-120, 120-130, 130-140, 140-150, 150-160, 150-200, 160-170,170-180, 180-190, 190-200, 200-210, 200-250, 210-220, 220.230, 230.240, 240.250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430.440, 440-450, 450-460, 450-500, 460-470, 470-480, 480-490, 490.500, 500.510, 500-550, 510-520, 520.530, 530-540, 540-550, 550-560, 550-600, 560.570, 570-580, 580-590, 590-600 and more than 600 nucleotides. As a non-limiting example, the viral genome comprises a promoter region that is about 260 nucleotides in length. As a non-limiting example, the viral genome comprises a promoter region that is about 283 nucleotides in length. As a non-limiting example, the viral genome comprises a promoter region that is about 299 nucleotides in length. As a non-limiting example, the viral genome comprises a promoter region that is about 365 nucleotides in length. As a non-limiting example, the viral genome comprises a promoter region that is about 380 nucleotides in length. As a non-limiting example, the viral genome comprises a promoter region that is about 382 nucleotides in length. As a non-limiting example, the viral genome comprises a promoter region that is about 557 nucleotides in length. As a non-limiting example, the viral genome comprises a promoter region that is about 654 nucleotides in length. As a non-limiting example, the viral genome comprises a promoter region that is about 699 nucleotides in length. As a non-limiting example, the viral genome comprises a promoter region that is about 1714 nucleotides in length. As a non-limiting example, the viral genome comprises a promoter region that is about 1715 nucleotides in length. As a non-limiting example, the viral genome comprises a promoter region that is about 1736 nucleotides in length.

In some embodiments, the AAV particle viral genome comprises at least one promoter sequence region. Non-limiting examples of promoter sequence regions are described in Table 18.

TABLE 18
Promoter Sequence Regions

| Sequence | Sequence | SEQ ID |
|---|---|---|
| Region Name | Length | NO |
| Promoter 1 | 1715 | 13523 |
| Promoter 2 | 299 | 13524 |
| Promoter 3 | 283 | 13525 |
| Promoter 4 | 260 | 13526 |
| Promoter 5 | 654 | 13527 |
| Promoter 6 | 699 | 13528 |
| Promoter 7 | 557 | 13529 |
| Promoter 8 | 382 | 13530 |
| Promoter 9 | 1736 | 13531 |
| Promoter 10 | 365 | 13532 |
| Promoter 11 | 1714 | 13533 |
| Promoter 12 | 380 | 13534 |

In some embodiments, the AAV particle viral genome comprises one promoter sequence region. In some embodiments, the promoter sequence region is Promoter 1. In some embodiments, the promoter sequence region is Promoter 2. In some embodiments, the promoter sequence region is Promoter 3. In some embodiments, the promoter sequence region is Promoter 4. In some embodiments, the promoter sequence region is Promoter 5. In some embodiments, the promoter sequence region is Promoter 6. In some embodiments, the promoter sequence region is Promoter 7. In some embodiments, the promoter sequence region is Promoter 8. In some embodiments, the promoter sequence region is Promoter 9. In some embodiments, the promoter sequence region is Promoter 10. In some embodiments, the promoter sequence region is Promoter 11. In some embodiments, the promoter sequence region is Promoter 12. In some embodiments, the promoter sequence region further comprises at least one promoter sub-region. As a non-limiting example, the promoter sequence is Promoter 1, further comprising Promoter 2 and Promoter 3 sub-regions.

In some embodiments, the AAV particle viral genome comprises more than one promoter sequence region. In some embodiments, the AAV particle viral genome comprises two promoter sequence regions. In some embodiments, the AAV particle viral genome comprises three promoter sequence regions.

In some embodiments, the AAV particle viral genome may comprise at least one exon sequence region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2.10, 5-10, 5-15, 10-20, 10-30, 10-40, 15-20, 15-25, 20-30, 20-40, 20-50, 25-30, 25-35, 30-40, 30-50, 30-60, 35.40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral genome comprises an exon region that is about 54 nucleotides in length. As a non-limiting example, the viral genome comprises an exon region that is about 59 nucleotides in length. As a non-limiting example, the viral genome comprises an exon region that is about 102 nucleotides in length. As a non-limiting example, the viral genome comprises an exon region that is about 134 nucleotides in length.

In some embodiments, the AAV particle viral genome comprises at least one Exon sequence region. Non-limiting examples of Exon sequence regions are described in Table 19.

TABLE 19
Exon Sequence Regions

| Sequence | Sequence | SEQ ID |
|---|---|---|
| Region Name | Length | NO |
| Exon1 | 134 | 13535 |
| Exon2 | 102 | 13536 |
| Exon3 | 59 | 13537 |
| Exon4 | 53 | 13538 |
| Exon5 | 54 | 13539 |

In some embodiments, the AAV particle viral genome comprises one Exon sequence region. In some embodiments, the Exon sequence region is the Exon1 sequence region. In some embodiments, the Exon sequence region is the Exon2 sequence region. In some embodiments, the Exon sequence region is the Exon3 sequence region. In some embodiments, the Exon sequence region is the Exon4 sequence region. In some embodiments, the Exon sequence region is the Exon5 sequence region.

In some embodiments, the AAV particle viral genome comprises two Exon sequence regions. In some embodiments, the AAV particle viral genome comprises three Exon sequence regions. In some embodiments, the AAV particle viral genome comprises four Exon sequence regions. In some embodiments, the AAV particle viral genome comprises more than four Exon sequence regions.

In some embodiments, the AAV particle viral genome may comprise at least one intron sequence region. The intron region(s) may, independently, have a length such as, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350 and more than 350 nucleotides. The length of the intron region for the viral genome may be 25-35, 25-50, 35-45, 45-55, 50-75, 55-65, 65-75, 75-85, 75.100, 85.95, 95-105,100-125, 105-115, 115-125,125-135, 125-150,135-145, 145-155,150-175, 155-165,165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 15 nucleotides in length. As a non-limiting example, the viral genome comprises an intron region that is about 32 nucleotides in length. As a non-limiting example, the viral genome comprises an intron region that is about 41 nucleotides in length. As a non-limiting example, the viral genome comprises an intron region that is about 53 nucleotides in length. As a non-limiting example, the viral genome comprises an intron region that is about 73 nucleotides in length. As a non-limiting example, the viral genome comprises an intron region that is about 168 nucleotides in length. As a non-limiting example, the viral genome comprises an intron region that is about 172 nucleotides in length. As a non-limiting example, the viral genome comprises an intron region that is about 292 nucleotides in length. As a non-limiting example, the viral genome comprises an intron region that is about 347 nucleotides in length. As a non-limiting example, the viral genome comprises an intron region that is about 387 nucleotides in length. As a non-limiting example, the viral genome comprises an intron region that is about 491 nucleotides in length. As a non-limiting example, the viral genome comprises an intron region that is about 566 nucleotides in length. As a non-limiting example, the viral genome comprises an intron region that is about 1074 nucleotides in length.

In some embodiments, the AAV particle viral genome comprises at least one intron sequence region. Non-limiting examples of intron sequence regions are described in Table 20.

TABLE 20
Intron Sequence Regions

| Sequence | Sequence | SEQ ID |
|---|---|---|
| Region Name | Length | NO |
| Intron1 | 32 | 13540 |
| Intron2 | 15 | 13541 |
| Intron3 | 347 | 13542 |
| Intron4 | 168 | 13543 |
| Intron5 | 73 | 13544 |
| Intron6 | 73 | 13545 |
| Intron7 | 73 | 13546 |
| Intron8 | 53 | 13547 |
| Intron9 | 172 | 13548 |
| Intron10 | 1074 | 13549 |
| Intron11 | 41 | 13550 |
| Intron12 | 566 | 13551 |
| Intron13 | 491 | 13552 |
| Intron14 | 387 | 13553 |
| Intron15 | 292 | 13554 |

In some embodiments, the AAV particle viral genome comprises one intron sequence region. In some embodiments, the intron sequence region is the Intron1 sequence region. In some embodiments, the intron sequence region is the Intron2 sequence region. In some embodiments, the intron sequence region is the Intron3 sequence region. In some embodiments, the intron sequence region is the Intron4 sequence region. In some embodiments, the intron sequence region is the Intron5 sequence region. In some embodiments, the intron sequence region is the Intron6 sequence region. In some embodiments, the intron sequence region is the Intron7 sequence region. In some embodiments, the intron sequence region is the Intron8 sequence region. In some embodiments, the intron sequence region is the Intron9 sequence region. In some embodiments, the intron sequence region is the Intron10 sequence region. In some embodiments, the intron sequence region is the Intron11 sequence region. In some embodiments, the intron sequence region is the Intron12 sequence region. In some embodiments, the intron sequence region is the Intron13 sequence region. In some embodiments, the intron sequence region is the Intron14 sequence region. In some embodiments, the intron sequence region is the Intron15 sequence region.

In some embodiments, the AAV particle viral genome comprises two intron sequence regions. In some embodiments, the AAV particle viral genome comprises three intron sequence regions. In some embodiments, the AAV particle viral genome comprises more than three intron sequence regions.

In some embodiments, the AAV particle viral genome may comprise at least one signal sequence region, not derived from an antibody. In another embodiment, the signal sequence region may be derived from an antibody sequence. The signal sequence region(s) may, independently, have a length such as, but not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the signal region in the viral genome may be 10-15, 15-25, 25-35, 25-50, 35.45, 45.55, 50.75, 55-65, 65.75, 75-85,75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225.235, 225-250, 235-245, 245.255, 250-275, 255.265, 265.275, 275.285, 275-300, 285.295, 295-305, 300-325, 305.315, 315-325, 325.335, 325-350, and 335-345 nucleotides. As a non-limiting example, the viral genome comprises a signal sequence region that is about 12 nucleotides in length. As a non-limiting example, the viral genome comprises a signal sequence region that is about 57 nucleotides in length. As a non-limiting example, the viral genome comprises a signal sequence region that is about 66 nucleotides in length. As a non-limiting example, the viral genome comprises a signal sequence region that is about 69 nucleotides in length. As a non-limiting example, the viral genome comprises a signal sequence region that is about 72 nucleotides in length. As a non-limiting example, the viral genome comprises a signal sequence region that is about 78 nucleotides in length. As a non-limiting example, the viral genome comprises a signal sequence region that is about 81 nucleotides in length. As a non-limiting example, the viral genome comprises a signal sequence region that is about 84 nucleotides in length. As a non-limiting example, the viral genome comprises a signal sequence region that is about 93 nucleotides in length. As a non-limiting example, the viral genome comprises a signal sequence region that is about 96 nucleotides in length. As a non-limiting example, the viral genome comprises a signal sequence region that is about 411 nucleotides in length.

In some embodiments, the AAV particle viral genome comprises at least one signal sequence region. Non-limiting examples of signal sequence regions not derived from an antibody sequence are described in Table 21.

TABLE 21
Signal Sequence Regions

| Sequence | Sequence | SEQ ID |
|---|---|---|
| Region Name | Length | NO |
| Signal1 | 84 | 13555 |
| Signal2 | 93 | 13556 |
| Signal3 | 96 | 13557 |
| Signal4 | 66 | 13558 |
| Signal5 | 72 | 13559 |
| Signal6 | 93 | 13560 |
| Signal7 | 69 | 13561 |
| Signal8 | 81 | 13562 |
| Signal9 | 12 | 13563 |
| Signal10 | 81 | 13564 |
| Signal11 | 66 | 13565 |
| Signal12 | 78 | 13566 |
| Signal13 | 57 | 13567 |
| Signal14 | 57 | 13568 |
| Signal15 | 57 | 13569 |
| Signal16 | 411 | 13570 |

In some embodiments, the AAV particle viral genome comprises one signal sequence region. In some embodiments, the signal sequence region is the Signal1 sequence region. In some embodiments, the signal sequence region is the Signal2 sequence region. In some embodiments, the signal sequence region is the Signal3 sequence region. In some embodiments, the signal sequence region is the Signal4 sequence region. In some embodiments, the signal sequence region is the Signal5 sequence region. In some embodiments, the signal sequence region is the Signal6 sequence region. In some embodiments, the signal sequence region is the Signal7 sequence region. In some embodiments, the signal sequence region is the Signal8 sequence region. In some embodiments, the signal sequence region is the Signal9 sequence region. In some embodiments, the signal sequence region is the Signal10 sequence region. In some embodiments, the signal sequence region is the Signal11 sequence region. In some embodiments, the signal sequence region is the Signal12 sequence region. In some embodiments, the signal sequence region is the Signal13 sequence region. In some embodiments, the signal sequence region is the Signal14 sequence region. In some embodiments, the signal sequence region is the Signal15 sequence region. In some embodiments, the signal sequence region is the Signal16 sequence region.

In some embodiments, the AAV particle viral genome comprises one signal sequence region. In some embodiments, the AAV particle viral genome comprises two signal sequence regions. In some embodiments, the AAV particle viral genome comprises three signal sequence regions. In some embodiments, the AAV particle viral genome comprises more than three signal sequence regions. In some embodiments, the signal sequences of a viral genome comprising more than one signal sequence, are the same. In another embodiment, the signal sequences of a viral genome comprising more than one signal sequence, are not the same.

In some embodiments, the AAV particle viral genome may comprise at least one tag sequence region. As used herein, the term “tag” indicates a polynucleotide sequence appended to the payload, that once expressed may be used to identify the expressed payload. Alternatively, the term “tag” may indicate a polynucleotide sequence appended to the payload that signals for retention of the expressed payload in a particular region of the cell (e.g., endoplasmic reticulum). The tag sequence region(s) may, independently, have a length such as, but not limited to, 10, 11, 12, 13, 14, 15, 16, 17, 18, 19, 20, 21, 22, 23, 24, 25, 26, 27, 28, 29, 30 or more than 30 nucleotides. The length of the tag sequence region in the viral genome may be 10-15, 15-20, 20-25, 25-30, or more than 30 nucleotides. As a non-limiting example, the viral genome comprises a tag sequence region that is about 18 nucleotides in length. As a non-limiting example, the viral genome comprises a tag sequence region that is about 21 nucleotides in length. As a non-limiting example, the viral genome comprises a tag sequence region that is about 27 nucleotides in length.

In some embodiments, the AAV particle viral genome comprises at least one tag sequence region. Non-limiting examples of tag sequence regions are described in Table 22.

TABLE 22
Tag Sequence Regions

| Sequence | Sequence | SEQ ID |
|---|---|---|
| Region Name | Length | NO |
| Tag1 | 27 | 13571 |
| Tag2 | 21 | 13572 |
| Tag3 | 18 | 13573 |
| Tag4 | 18 | 13574 |
| Tag5 | 18 | 13575 |

In some embodiments, the AAV particle viral genome comprises one tag sequence region. In some embodiments, the tag sequence region is the Tag1 sequence region. In some embodiments, the tag sequence region is the Tag2 sequence region. In some embodiments, the tag sequence region is the Tag3 sequence region. In some embodiments, the tag sequence region is the Tag4 sequence region. In some embodiments, the tag sequence region is the Tag5 sequence region.

In some embodiments, the AAV particle viral genome comprises more than one tag sequence region. In some embodiments, the AAV particle viral genome comprises two tag sequence regions. In some embodiments, the AAV particle viral genome comprises three tag sequence regions. In some embodiments, the AAV particle viral genome comprises more than three tag sequence regions.

In some embodiments, the AAV particle viral genome may comprise at least one polyadenylation sequence region. The polyadenylation sequence region(s) may, independently, have a length such as, but not limited to, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119,120, 121,122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the polyadenylation sequence region for the viral genome may be 4-10, 10-20, 10-50, 20-30, 30-40, 40-50, 50-60, 50-100, 60-70, 70-80, 80-90, 90-100,100-110, 100-150, 110-120,120-130, 130-140,140-150, 150-160, 150-200, 160-170, 170-180, 180-190, 190-200, 200-210, 200-250, 210.220, 220.230, 230.240, 240-250, 250-260, 250-300, 260-270, 270-280, 280-290, 290-300, 300-310, 300-350, 310-320, 320-330, 330-340, 340-350, 350-360, 350.400, 360-370, 370-380, 380-390, 390.400, 400.410, 400.450, 410.420, 420.430, 430-440, 440-450, 450.460, 450-500, 460.470, 470-480, 480.490, 490-500, 500-510, 500-550, 510.520, 520-530, 530.540, 540.550, 550-560, 550-600, 560-570, 570.580, 580-590, and 590-600 nucleotides. As a non-limiting example, the viral genome comprises a polyadenylation sequence region that is about 127 nucleotides in length. As a non-limiting example, the viral genome comprises a polyadenylation sequence region that is about 477 nucleotides in length. As a non-limiting example, the viral genome comprises a polyadenylation sequence region that is about 552 nucleotides in length.

In some embodiments, the AAV particle viral genome comprises at least one polyadenylation (polyA) sequence region. Non-limiting examples of polyA sequence regions are described in Table 23.

TABLE 23
PolyA Signal Sequence Regions

| Sequence | Sequence | SEQ ID |
|---|---|---|
| Region Name | Length | NO |
| PolyA1 | 127 | 13576 |
| PolyA2 | 477 | 13577 |
| PolyA3 | 552 | 13578 |

In some embodiments, the AAV particle viral genome comprises one polyA sequence region. In some embodiments, the polyA sequence region is the PolyA1 sequence. In some embodiments, the polyA sequence region is the PolyA2 sequence. In some embodiments, the polyA signal sequence region is the PolyA3 sequence.

In some embodiments, the AAV particle viral genome comprises more than one polyA sequence region.

In some embodiments, the AAV particle viral genome may comprise at least one or multiple filler sequence regions. The filler region(s) may, independently, have a length such as, but not limited to, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114,115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185,186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 256, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800.850, 850-900, 900-950, 950-1000, 1000-1050, 1050-1100, 1100-1150, 1150-1200, 1200.1250, 1250-1300,1300-1350, 1350-1400, 1400-1450, 1450-1500,1500-1550, 1550-1600, 1600-1650,1650-1700, 1700-1750, 1750-1800, 1800-1850, 1850-1900, 1900-1950, 1950-2000, 2000-2050, 2050-2100, 2100.2150, 2150.2200, 2200.2250, 2250-2300, 2300-2350, 2350.2400, 2400.2450, 2450.2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As a non-limiting example, the viral genome comprises a filler region that is about 1153 nucleotides in length. As a non-limiting example, the viral genome comprises a filler region that is about 1240 nucleotides in length.

In some embodiments, the AAV particle viral genome comprises at least one filler sequence region. Non-limiting examples of filler sequence regions are described in Table 24.

TABLE 24
Filler Sequence Regions

| Sequence | Sequence | SEQ ID |
|---|---|---|
| Region Name | Length | NO |
| FILLER1 | 1153 | 13579 |
| FILLER2 | 1240 | 13580 |

In some embodiments, the AAV particle viral genome comprises filler sequence region FILLER1. In some embodiments, the AAV particle viral genome comprises filler sequence region FILLER2. In some embodiments, the AAV particle viral genome comprises both FILLER1 and FILLER2. In some embodiments, the AAV particle viral genome does not comprise a filler sequence region.

### Regions of Viral Genome Cassettes

In some embodiments, the viral genome comprises at least one sequence region described in Tables 17-24.

### Viral Genome Cassette Sequence Regions: 5′ITR, 3′ITR, Promoter, PolyA

In some embodiments, the viral genome comprises a 5′ITR and 3′ITR sequence region, at least one promoter sequence region, a polyA sequence region and at least one payload region. The 5′ITR and 3′ITR sequence region may be selected from Table 17, at least one of the promoter sequence regions may be selected from Table 18, and the polyA sequence region may be selected from Table 23. The ITR sequence regions may be located on the 5 and 3′ termini of the construct, the promoter is located upstream of the payload region and the polyA sequence region may be located upstream of the 3′ITR sequence region.

In some embodiments, the viral genome comprises a 5′ITR and 3′ITR sequence region, at least one promoter sequence region, a polyA sequence region, a filler sequence region and at least one payload region. The 5′ITR and 3′ITR sequence region may be selected from Table 17, at least one of the promoter sequence regions may be selected from Table 18, the polyA sequence region may be selected from Table 23, and the filler sequence region may be selected from Table 24. The ITR sequence regions may be located on the 5′ and 3′ termini of the construct, the promoter is located upstream of the payload region, the polyA sequence region may be located upstream of the 3′ITR sequence region, and the filler sequence region may be located between the polyA sequence region and the 3′ITR sequence region.

As a non-limiting example, the 5′ITR is ITR1 (SEQ ID NO: 13519), the 3′ITR is ITR3 (SEQ ID NO: 13521), the promoter is PROMOTER 1 (SEQ ID NO: 13523), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 1 (SEQ ID NO: 13523), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 1 (SEQ ID NO: 13523), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 2 (SEQ ID NO: 13524), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 2 (SEQ ID NO: 13524), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 2 (SEQ ID NO: 13524), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 3 (SEQ ID NO: 13525), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 3 (SEQ ID NO: 13525), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 3 (SEQ ID NO: 13525), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 5 (SEQ ID NO: 13527), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 5 (SEQ ID NO: 13527), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 5 (SEQ ID NO: 13527), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 6 (SEQ ID NO: 13528), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 6 (SEQ ID NO: 13528), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 6 (SEQ ID NO: 13528), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 7 (SEQ ID NO: 13529), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 7 (SEQ ID NO: 13529), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 7 (SEQ ID NO: 13529), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 8 (SEQ ID NO: 13530), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 8 (SEQ ID NO: 13530), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 8 (SEQ ID NO: 13530), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 9 (SEQ ID NO: 13531), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 9 (SEQ ID NO: 13531), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 9 (SEQ ID NO: 13531), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 10 (SEQ ID NO: 13532), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 10 (SEQ ID NO: 13532), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 10 (SEQ ID NO: 13532), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 11 (SEQ ID NO: 13533), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 11 (SEQ ID NO: 13533), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 11 (SEQ ID NO: 13533), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 12 (SEQ ID NO: 13534), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 12 (SEQ ID NO: 13534), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 12 (SEQ ID NO: 13534), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526) and PROMOTER 8 (SEQ ID NO: 13530), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526) and PROMOTER 8 (SEQ ID NO: 13530), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526) and PROMOTER 8 (SEQ ID NO: 13530), and the polyA sequence is POLYA3 (SEQ ID NO: 13578). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526) and PROMOTER 12 (SEQ ID NO: 13534), and the polyA sequence is POLYA1 (SEQ ID NO: 13576). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526) and PROMOTER 12 (SEQ ID NO: 13534), and the polyA sequence is POLYA2 (SEQ ID NO: 13577). As a non-limiting example, the 5′ITR is ITR1 (SEQ ID NO: 13519), the 3′ITR is ITR3 (SEQ ID NO: 13521), the promoter is PROMOTER 4 (SEQ ID NO: 13526) and PROMOTER 12 (SEQ ID NO: 13534), and the polyA sequence is POLYA3 (SEQ ID NO: 13578).

As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523), and the polyA sequence is POLYA1 (SEQ ID NO: 13576). As a non-limiting example, the 5′ITR is ITR1 (SEQ ID NO: 13519), the 3′ITR1s ITR4 (SEQ ID NO: 13522), the promoter is PROMOTER 1 (SEQ ID NO: 13523), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 2 (SEQ ID NO: 13524), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 2 (SEQ ID NO: 13524), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 2 (SEQ ID NO: 13524), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 3 (SEQ ID NO: 13525), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 3 (SEQ ID NO: 13525), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 3 (SEQ ID NO: 13525), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 5 (SEQ ID NO: 13527), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 5 (SEQ ID NO: 13527), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 5 (SEQ ID NO: 13527), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 6 (SEQ ID NO: 13528), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 6 (SEQ ID NO: 13528), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 6 (SEQ ID NO: 13528), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 7 (SEQ ID NO: 13529), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 7 (SEQ ID NO: 13529), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 7 (SEQ ID NO: 13529), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 8 (SEQ ID NO: 13530), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 8 (SEQ ID NO: 13530), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 8 (SEQ ID NO: 13530), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 9 (SEQ ID NO: 13531), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 9 (SEQ ID NO: 13531), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 9 (SEQ ID NO: 13531), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 10 (SEQ ID NO: 13532), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 10 (SEQ ID NO: 13532), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 10 (SEQ ID NO: 13532), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 11 (SEQ ID NO: 13533), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 11 (SEQ ID NO: 13533), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 11 (SEQ ID NO: 13533), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 12 (SEQ ID NO: 13534), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 12 (SEQ ID NO: 13534), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 12 (SEQ ID NO: 13534), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 8 (SEQ ID NO: 13530), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 8 (SEQ ID NO: 13530), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 8 (SEQ ID NO: 13530), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 12 (SEQ ID NO: 13534), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 12 (SEQ ID NO: 13534), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 12 (SEQ ID NO: 13534), and the polyA sequence is POLYA3 (SEQ ID NO: 13578).

As a non-limiting example, the 5′ITR is ITR2 (SEQ ID NO: 13520), the 3′ITR is ITR3 (SEQ ID NO: 13521), the promoter is PROMOTER 1 (SEQ ID NO: 13523), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 1 (SEQ ID NO: 13523), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 1 (SEQ ID NO: 13523), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 2 (SEQ ID NO: 13524), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 2 (SEQ ID NO: 13524), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 2 (SEQ ID NO: 13524), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 3 (SEQ ID NO: 13525), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 3 (SEQ ID NO: 13525), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 3 (SEQ ID NO: 13525), and the polyA sequence is POLYA3 (SEQ ID NO: 13578), As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 5 (SEQ ID NO: 13527), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 5 (SEQ ID NO: 13527), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 5 (SEQ ID NO: 13527), and the polyA sequence is POLYA3 (SEQ ID NO: 13578), As a non-limiting example, the 5′ITR is ITR2 (SEQ ID NO: 13520), the ITR is ITR3 (SEQ ID NO: 13521), the promoter is PROMOTER 6 (SEQ ID NO: 13528), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 6 (SEQ ID NO: 13528), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 6 (SEQ ID NO: 13528), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 7 (SEQ ID NO: 13529), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 7 (SEQ ID NO: 13529), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 7 (SEQ ID NO: 13529), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 8 (SEQ ID NO: 13530), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 8 (SEQ ID NO: 13530), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 8 (SEQ ID NO: 13530), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 9 (SEQ ID NO: 13531), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 9 (SEQ ID NO: 13531), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 9 (SEQ ID NO: 13531), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 10 (SEQ ID NO: 13532), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 10 (SEQ ID NO: 13532), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 10 (SEQ ID NO: 13532), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 11 (SEQ ID NO: 13533), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 11 (SEQ ID NO: 13533), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 11 (SEQ ID NO: 13533), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 12 (SEQ ID NO: 13534), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 12 (SEQ ID NO: 13534), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 12 (SEQ ID NO: 13534), and the polyA sequence is POLYA3 (SEQ ID NO: 13578). As a non-limiting example, the 5′ITR is ITR2 (SEQ ID NO: 13520), the ITR is ITR3 (SEQ ID NO: 13521), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526) and PROMOTER 8 (SEQ ID NO: 13530), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526) and PROMOTER 8 (SEQ ID NO: 13530), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526) and PROMOTER 8 (SEQ ID NO: 13530), and the polyA sequence is POLYA3 (SEQ ID NO: 13578). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526) and PROMOTER 12 (SEQ ID NO: 13534), and the polyA sequence is POLYA1 (SEQ ID NO: 13576). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526) and PROMOTER 12 (SEQ ID NO: 13534), and the polyA sequence is POLYA2 (SEQ ID NO: 13577). As a non-limiting example, the 5′ITR is ITR2 (SEQ ID NO: 13520), the 3′ITR is ITR3 (SEQ ID NO: 13521), the promoter is PROMOTER 4 (SEQ ID NO: 13526) and PROMOTER 12 (SEQ ID NO: 13534), and the polyA sequence is POLYA3 (SEQ ID NO: 13578).

As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 2 (SEQ ID NO: 13524), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 2 (SEQ ID NO: 13524), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 2 (SEQ ID NO: 13524), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 3 (SEQ ID NO: 13525), and the polyA sequence is POLYA1 (SEQ ID NO: 13576). As a non-limiting example, the 5′ITR is ITR1 (SEQ ID NO: 13519), the 3′1′ITR is ITR4 (SEQ ID NO: 13522), the promoter is PROMOTER 3 (SEQ ID NO: 13525), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 3 (SEQ ID NO: 13525), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 5 (SEQ ID NO: 13527), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 5 (SEQ ID NO: 13527), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 5 (SEQ ID NO: 13527), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 6 (SEQ ID NO: 13528), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 6 (SEQ ID NO: 13528), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 6 (SEQ ID NO: 13528), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 7 (SEQ ID NO: 13529), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 7 (SEQ ID NO: 13529), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 7 (SEQ ID NO: 13529), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 8 (SEQ ID NO: 13530), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 8 (SEQ ID NO: 13530), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 8 (SEQ ID NO: 13530), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 9 (SEQ ID NO: 13531), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 9 (SEQ ID NO: 13531), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 9 (SEQ ID NO: 13531), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 10 (SEQ ID NO: 13532), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 10 (SEQ ID NO: 13532), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 10 (SEQ ID NO: 13532), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 11 (SEQ ID NO: 13533), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 11 (SEQ ID NO: 13533), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 11 (SEQ ID NO: 13533), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 12 (SEQ ID NO: 13534), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 12 (SEQ ID NO: 13534), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 12 (SEQ ID NO: 13534), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER1 (SEQ ID NO: 13523) which comprises PROMOTER 2 (SEQ ID NO: 13524) and PROMOTER 3 (SEQ ID NO: 13525), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 1 (SEQ ID NO: 13523) which comprises PROMOTER 2 (SEQ ID NO: 13524) and PROMOTER 3 (SEQ ID NO: 13525), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 8 (SEQ ID NO: 13530), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 8 (SEQ ID NO: 13530), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 8 (SEQ ID NO: 13530), and the polyA sequence is POLYA3 (SEQ ID NO: 13578). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 12 (SEQ ID NO: 13534), and the polyA sequence is POLYA1 (SEQ ID NO: 13576). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 12 (SEQ ID NO: 13534), and the polyA sequence is POLYA2 (SEQ ID NO: 13577). As a non-limiting example, the 5′ITR is ITR1 (SEQ ID NO: 13519), the 3′ITR is ITR4 (SEQ ID NO: 13522), the promoter is PROMOTER 4 (SEQ ID NO: 13526) and PROMOTER 12 (SEQ ID NO: 13534), and the polyA sequence is POLYA3 (SEQ ID NO: 13578).

### Viral Genome Cassette Sequence Regions: Exon and Intron Sequences

In some embodiments, the viral genome comprises a 5′ITR and 3′ITR sequence region, at least one promoter sequence region, at least one exon sequence region, at least one intron sequence region, at least one a polyA sequence region and at least one payload region. The 5′ITR and 3′ITR sequence region may be selected from Table 17, at least one of the promoter sequence regions may be selected from Table 18, at least one of the exon sequence regions may be selected from Table 19, at least one of the intron sequence regions may be selected from Table 20, the polyA sequence region may be selected from Table 23. The ITR sequence regions may be located on the 5′ and 3′ termini of the construct, the promoter is located upstream of the payload region, the polyA sequence region may be located upstream of the 3′ITR sequence region, and the filler sequence region may be located between the polyA sequence region and the 3′ITR sequence region.

In some embodiments, the viral genome comprises a 5′ITR and 3′ITR sequence region, at least one promoter sequence region, at least one intron sequence region, at least one a polyA sequence region and at least one payload region. The 5′ITR and 3′ITR sequence region may be selected from Table 17, at least one of the promoter sequence regions may be selected from Table 18, at least one of the intron sequence regions may be selected from Table 20, the polyA sequence region may be selected from Table 23. The ITR sequence regions may be located on the 5 and 3′ termini of the construct, the promoter is located upstream of the payload region, the polyA sequence region may be located upstream of the 3′ITR sequence region, and the filler sequence region may be located between the polyA sequence region and the 3′ITR sequence region. As a non-limiting example, the intron is INTRON1 (SEQ ID NO: 13540). As a non-limiting example, the intron is INTRON2 (SEQ ID NO: 13541). As a non-limiting example, the intron is INTRON3 (SEQ ID NO: 13542). As a non-limiting example, the intron is INTRON4 (SEQ ID NO: 13543). As a non-limiting example, the intron is INTRON5 (SEQ ID NO: 13544). As a non-limiting example, the intron is INTRON6 (SEQ ID NO: 13545). As a non-limiting example, the intron is INTRON7 (SEQ ID NO: 13546). As a non-limiting example, the intron is INTRONS (SEQ ID NO: 13547). As a non-limiting example, the intron is INTRON9 (SEQ ID NO: 13548). As a non-limiting example, the intron is INTRON10 (SEQ ID NO: 13549). As a non-limiting example, the intron is INTRON11 (SEQ ID NO: 13550). As a non-limiting example, the intron is INTRON12 (SEQ ID NO: 13551). As a non-limiting example, the intron is INTRON13 (SEQ ID NO: 13552). As a non-limiting example, the intron is INTRON14 (SEQ ID NO: 13553). As a non-limiting example, the intron is INTRON15 (SEQ ID NO: 13554).

### Viral Genome Cassette Sequence Regions: Filler Sequence

The viral genome may also optionally comprise a filler sequence region. Non-limiting examples of filler sequence regions are described in Table 24. As a non-limiting example, the viral genome comprises FILLER1 (SEQ ID NO: 13579). As a non-limiting example, the viral genome comprises FILLER2 (SEQ ID NO: 13580).

### Viral Genome Cassette Sequence Regions: Tag Sequence

In some embodiments, the viral genome cassette may also comprise a tag sequence region. The tag sequence may be located upstream of the polyA sequence region. The tag sequence region may be selected from Table 22. As a non-limiting example, the tag sequence region is TAG1 (SEQ ID NO: 13571). As a non-limiting example, the tag sequence region is TAG2 (SEQ ID NO: 13572). As a non-limiting example, the tag sequence region is TAG3 (SEQ ID NO: 13573). As a non-limiting example, the tag sequence region is TAG4 (SEQ ID NO: 13574).

### Viral Genome Cassette Sequence Regions: Signal Sequence

In some embodiments, the viral genome cassette may also comprise at least one signal sequence region. The signal sequence region may be located upstream of the payload region and, if there is more than one payload region, the signal sequence may be located upstream to some or all of the payload regions. If there is more than one signal sequence regions in the viral genome, the signal sequence regions may be the same or different. The signal sequence region may be selected from Table 21. As a non-limiting example, the signal sequence region is SIGNAL1 (SEQ ID NO: 13555). As a non-limiting example, the signal sequence region is SIGNAL2 (SEQ ID NO: 13556). As a non-limiting example, the signal sequence region is SIGNAL3 (SEQ ID NO: 13557). As a non-limiting example, the signal sequence region is SIGNAL4 (SEQ ID NO: 13558). As a non-limiting example, the signal sequence region is SIGNALS (SEQ ID NO: 13559). As a non-limiting example, the signal sequence region is SIGNALS (SEQ ID NO: 13560). As a non-limiting example, the signal sequence region is SIGNAL7 (SEQ ID NO: 13561). As a non-limiting example, the signal sequence region is SIGNALS (SEQ ID NO: 13562). As a non-limiting example, the signal sequence region is SIGNAL9 (SEQ ID NO: 13563). As a non-limiting example, the signal sequence region is SIGNAL10 (SEQ ID NO: 13564). As a non-limiting example, the signal sequence region is SIGNAL11 (SEQ ID NO: 13565). As a non-limiting example, the signal sequence region is SIGNAL12 (SEQ ID NO: 13566). As a non-limiting example, the signal sequence region is SIGNAL13 (SEQ ID NO: 13567). As a non-limiting example, the signal sequence region is SIGNAL14 (SEQ ID NO: 13568). As a non-limiting example, the signal sequence region is SIGNAL15 (SEQ ID NO: 13569). As a non-limiting example, the signal sequence region is SIGNAL16 (SEQ ID NO: 13570).

In some embodiments, the viral genome cassette may also comprise two signal sequence regions. The payload region of the viral genome may have one of the two signal sequences located upstream of the payload region and the signal sequences may be the same or different sequences. As a non-limiting example, the viral genome comprises two signal sequence regions and the two signal sequence regions are: SIGNAL1 (SEQ ID NO: 13555) and SIGNAL2 (SEQ ID NO: 13556). As a non-limiting example, the viral genome comprises two signal sequence regions and the two signal sequence regions are: SIGNAL1 (SEQ ID NO: 13555) and SIGNAL3 (SEQ ID NO: 13557). As a non-limiting example, the viral genome comprises two signal sequence regions and the two signal sequence regions are: SIGNAL1 (SEQ ID NO: 13555) and SIGNAL4 (SEQ ID NO: 13558). As a non-limiting example, the viral genome comprises two signal sequence regions and the two signal sequence regions are: SIGNAL1 (SEQ ID NO: 13555) and SIGNALS (SEQ ID NO: 13559). As a non-limiting example, the viral genome comprises two signal sequence regions and the two signal sequence regions are: SIGNAL1 (SEQ ID NO: 13555) and SIGNALS (SEQ ID NO: 13560). As a non-limiting example, the viral genome comprises two signal sequence regions and the two signal sequence regions are: SIGNAL1 (SEQ ID NO: 13555) and SIGNAL7 (SEQ ID NO: 13561). As a non-limiting example, the viral genome comprises two signal sequence regions and the two signal sequence regions are: SIGNAL1 (SEQ ID NO: 13555) and SIGNALS (SEQ ID NO: 13562). As a non-limiting example, the viral genome comprises two signal sequence regions and the two signal sequence regions are: SIGNAL1 (SEQ ID NO: 13555) and SIGNAL9 (SEQ ID NO: 13563). As a non-limiting example, the viral genome comprises two signal sequence regions and the two signal sequence regions are: SIGNAL1 (SEQ ID NO: 13555) and SIGNAL10 (SEQ ID NO: 13564). As a non-limiting example, the viral genome comprises two signal sequence regions and the two signal sequence regions are: SIGNAL1 (SEQ ID NO: 13555) and SIGNAL11 (SEQ ID NO: 13565). As a non-limiting example, the viral genome comprises two signal sequence regions and the two signal sequence regions are: SIGNAL1 (SEQ ID NO: 13555) and SIGNAL12 (SEQ ID NO: 13566). As a non-limiting example, the viral genome comprises two signal sequence regions and the two signal sequence regions are: SIGNAL1 (SEQ ID NO: 13555) and SIGNAL13 (SEQ ID NO: 13567). As a non-limiting example, the viral genome comprises two signal sequence regions and the two signal sequence regions are: SIGNAL1 (SEQ ID NO: 13555) and SIGNAL14 (SEQ ID NO: 13568). As a non-limiting example, the viral genome comprises two signal sequence regions and the two signal sequence regions are: SIGNAL1 (SEQ ID NO: 13555) and SIGNAL15 (SEQ ID NO: 13569). As a non-limiting example, the viral genome comprises two signal sequence regions and the two signal sequence regions are: SIGNAL1 (SEQ ID NO: 13555) and SIGNAL16 (SEQ ID NO: 13570). As a non-limiting example, the viral genome comprises two signal sequence regions and the two signal sequence regions are: SIGNAL2 (SEQ ID NO: 13556) and SIGNAL3 (SEQ ID NO: 13557). As a non-limiting example, the viral genome comprises two signal sequence regions and the two signal sequence regions are: SIGNAL2 (SEQ ID NO: 13556) and SIGNAL4 (SEQ ID NO: 13558). As a non-limiting example, the viral genome comprises two signal sequence regions and the two signal sequence regions are: SIGNAL2 (SEQ ID NO: 13556) and SIGNAL5 (SEQ ID NO: 13559). As a non-limiting example, the viral genome comprises two signal sequence regions and the two signal sequence regions are: SIGNAL2 (SEQ ID NO: 13556) and SIGNALS (SEQ ID NO: 13560). As a non-limiting example, the viral genome comprises two signal sequence regions and the two signal sequence regions are: SIGNAL2 (SEQ ID NO: 13556) and SIGNAL7 (SEQ ID NO: 13561). As a non-limiting example, the viral genome comprises two signal sequence regions and the two signal sequence regions are: SIGNAL2 (SEQ ID NO: 13556) and SIGNAL8 (SEQ ID NO: 13562). As a non-limiting example, the viral genome comprises two signal sequence regions and the two signal sequence regions are: SIGNAL2 (SEQ ID NO: 13556) and SIGNAL9 (SEQ ID NO: 13563). As a non-limiting example, the viral genome comprises two signal sequence regions and the two signal sequence regions are: SIGNAL2 (SEQ ID NO: 13556) and SIGNAL10 (SEQ ID NO: 13564). As a non-limiting example, the viral genome comprises two signal sequence regions and the two signal sequence regions are: SIGNAL2 (SEQ ID NO: 13556) and SIGNAL11 (SEQ ID NO: 13565). As a non-limiting example, the viral genome comprises two signal sequence regions and the two signal sequence regions are: SIGNAL2 (SEQ ID NO: 13556) and SIGNAL12 (SEQ ID NO: 13566). As a non-limiting example, the viral genome comprises two signal sequence regions and the two signal sequence regions are: SIGNAL2 (SEQ ID NO: 13556) and SIGNAL13 (SEQ ID NO: 13567). As a non-limiting example, the viral genome comprises two signal sequence regions and the two signal sequence regions are: SIGNAL2 (SEQ ID NO: 13556) and SIGNAL14 (SEQ ID NO: 13568). As a non-limiting example, the viral genome comprises two signal sequence regions and the two signal sequence regions are: SIGNAL2 (SEQ ID NO: 13556) and SIGNAL15 (SEQ ID NO: 13569). As a non-limiting example, the viral genome comprises two signal sequence regions and the two signal sequence regions are: SIGNAL2 (SEQ ID NO: 13556) and SIGNAL16 (SEQ ID NO: 13570). As a non-limiting example, the viral genome comprises two signal sequence regions and the two signal sequence regions are: SIGNAL3 (SEQ ID NO: 13557) and SIGNAL4 (SEQ ID NO: 13558). As a non-limiting example, the viral genome comprises two signal sequence regions and the two signal sequence regions are: SIGNAL3 (SEQ ID NO: 13557) and SIGNALS (SEQ ID NO: 13559). As a non-limiting example, the viral genome comprises two signal sequence regions and the two signal sequence regions are: SIGNAL3 (SEQ ID NO: 13557) and SIGNALS (SEQ ID NO: 13560). As a non-limiting example, the viral genome comprises two signal sequence regions and the two signal sequence regions are: SIGNAL3 (SEQ ID NO: 13557) and SIGNAL7 (SEQ ID NO: 13561). As a non-limiting example, the viral genome comprises two signal sequence regions and the two signal sequence regions are: SIGNAL3 (SEQ ID NO: 13557) and SIGNALS (SEQ ID NO: 13562). As a non-limiting example, the viral genome comprises two signal sequence regions and the two signal sequence regions are: SIGNAL3 (SEQ ID NO: 13557) and SIGNAL9 (SEQ ID NO: 13563). As a non-limiting example, the viral genome comprises two signal sequence regions and the two signal sequence regions are: SIGNAL3 (SEQ ID NO: 13557) and SIGNAL11 (SEQ ID NO: 13564). As a non-limiting example, the viral genome comprises two signal sequence regions and the two signal sequence regions are: SIGNAL3 (SEQ ID NO: 13557) and SIGNAL11 (SEQ ID NO: 13565). As a non-limiting example, the viral genome comprises two signal sequence regions and the two signal sequence regions are: SIGNAL3 (SEQ ID NO: 13557) and SIGNAL12 (SEQ ID NO: 13566). As a non-limiting example, the viral genome comprises two signal sequence regions and the two signal sequence regions are: SIGNAL3 (SEQ ID NO: 13557) and SIGNAL13 (SEQ ID NO: 13567). As a non-limiting example, the viral genome comprises two signal sequence regions and the two signal sequence regions are: SIGNAL3 (SEQ ID NO: 13557) and SIGNAL14 (SEQ ID NO: 13568). As a non-limiting example, the viral genome comprises two signal sequence regions and the two signal sequence regions are: SIGNAL3 (SEQ ID NO: 13557) and SIGNAL15 (SEQ ID NO: 13569). As a non-limiting example, the viral genome comprises two signal sequence regions and the two signal sequence regions are: SIGNAL3 (SEQ ID NO: 13557) and SIGNAL16 (SEQ ID NO: 13570). As a non-limiting example, the viral genome comprises two signal sequence regions and the two signal sequence regions are: SIGNAL4 (SEQ ID NO: 13558) and SIGNALS (SEQ ID NO: 13559). As a non-limiting example, the viral genome comprises two signal sequence regions and the two signal sequence regions are: SIGNAL4 (SEQ ID NO: 13558) and SIGNALS (SEQ ID NO: 13560). As a non-limiting example, the viral genome comprises two signal sequence regions and the two signal sequence regions are: SIGNAL4 (SEQ ID NO: 13558) and SIGNAL7 (SEQ ID NO: 13561). As a non-limiting example, the viral genome comprises two signal sequence regions and the two signal sequence regions are: SIGNAL4 (SEQ ID NO: 13558) and SIGNALS (SEQ ID NO: 13562). As a non-limiting example, the viral genome comprises two signal sequence regions and the two signal sequence regions are: SIGNAL4 (SEQ ID NO: 13558) and SIGNAL9 (SEQ ID NO: 13563). As a non-limiting example, the viral genome comprises two signal sequence regions and the two signal sequence regions are: SIGNAL4 (SEQ ID NO: 13558) and SIGNAL10 (SEQ ID NO: 13564). As a non-limiting example, the viral genome comprises two signal sequence regions and the two signal sequence regions are: SIGNAL4 (SEQ ID NO: 13558) and SIGNAL11 (SEQ ID NO: 13565). As a non-limiting example, the viral genome comprises two signal sequence regions and the two signal sequence regions are: SIGNAL4 (SEQ ID NO: 13558) and SIGNAL12 (SEQ ID NO: 13566). As a non-limiting example, the viral genome comprises two signal sequence regions and the two signal sequence regions are: SIGNAL4 (SEQ ID NO: 13558) and SIGNAL13 (SEQ ID NO: 13567). As a non-limiting example, the viral genome comprises two signal sequence regions and the two signal sequence regions are: SIGNAL4 (SEQ ID NO: 13558) and SIGNAL14 (SEQ ID NO: 13568). As a non-limiting example, the viral genome comprises two signal sequence regions and the two signal sequence regions are: SIGNAL4 (SEQ ID NO: 13558) and SIGNAL15 (SEQ ID NO: 13569). As a non-limiting example, the viral genome comprises two signal sequence regions and the two signal sequence regions are: SIGNAL4 (SEQ ID NO: 13558) and SIGNAL16 (SEQ ID NO: 13570). As a non-limiting example, the viral genome comprises two signal sequence regions and the two signal sequence regions are: SIGNALS (SEQ ID NO: 13559) and SIGNALS (SEQ ID NO: 13560). As a non-limiting example, the viral genome comprises two signal sequence regions and the two signal sequence regions are: SIGNALS (SEQ ID NO: 13559) and SIGNAL7 (SEQ ID NO: 13561). As a non-limiting example, the viral genome comprises two signal sequence regions and the two signal sequence regions are: SIGNALS (SEQ ID NO: 13559) and SIGNAL8 (SEQ ID NO: 13562). As a non-limiting example, the viral genome comprises two signal sequence regions and the two signal sequence regions are: SIGNALS (SEQ ID NO: 13559) and SIGNAL9 (SEQ ID NO: 13563). As a non-limiting example, the viral genome comprises two signal sequence regions and the two signal sequence regions are: SIGNALS (SEQ ID NO: 13559) and SIGNAL10 (SEQ ID NO: 13564). As a non-limiting example, the viral genome comprises two signal sequence regions and the two signal sequence regions are: SIGNALS (SEQ ID NO: 13559) and SIGNAL11 (SEQ ID NO: 13565). As a non-limiting example, the viral genome comprises two signal sequence regions and the two signal sequence regions are: SIGNALS (SEQ ID NO: 13559) and SIGNAL12 (SEQ ID NO: 13566). As a non-limiting example, the viral genome comprises two signal sequence regions and the two signal sequence regions are: SIGNALS (SEQ ID NO: 13559) and SIGNAL13 (SEQ ID NO: 13567). As a non-limiting example, the viral genome comprises two signal sequence regions and the two signal sequence regions are: SIGNALS (SEQ ID NO: 13559) and SIGNAL14 (SEQ ID NO: 13568). As a non-limiting example, the viral genome comprises two signal sequence regions and the two signal sequence regions are: SIGNALS (SEQ ID NO: 13559) and SIGNAL15 (SEQ ID NO: 13569). As a non-limiting example, the viral genome comprises two signal sequence regions and the two signal sequence regions are: SIGNALS (SEQ ID NO: 13559) and SIGNAL16 (SEQ ID NO: 13570). As a non-limiting example, the viral genome comprises two signal sequence regions and the two signal sequence regions are: SIGNALS (SEQ ID NO: 13560) and SIGNAL7 (SEQ ID NO: 13561). As a non-limiting example, the viral genome comprises two signal sequence regions and the two signal sequence regions are: SIGNALS (SEQ ID NO: 13560) and SIGNAL8 (SEQ ID NO: 13562). As a non-limiting example, the viral genome comprises two signal sequence regions and the two signal sequence regions are: SIGNALS (SEQ ID NO: 13560) and SIGNAL9 (SEQ ID NO: 13563). As a non-limiting example, the viral genome comprises two signal sequence regions and the two signal sequence regions are: SIGNALS (SEQ ID NO: 13560) and SIGNAL10 (SEQ ID NO: 13564). As a non-limiting example, the viral genome comprises two signal sequence regions and the two signal sequence regions are: SIGNALS (SEQ ID NO: 13560) and SIGNAL11 (SEQ ID NO: 13565). As a non-limiting example, the viral genome comprises two signal sequence regions and the two signal sequence regions are: SIGNAL6 (SEQ ID NO: 13560) and SIGNAL12 (SEQ ID NO: 13566). As a non-limiting example, the viral genome comprises two signal sequence regions and the two signal sequence regions are: SIGNALS (SEQ ID NO: 13560) and SIGNAL13 (SEQ ID NO: 13567). As a non-limiting example, the viral genome comprises two signal sequence regions and the two signal sequence regions are: SIGNAL6 (SEQ ID NO: 13560) and SIGNAL14 (SEQ ID NO: 13566). As a non-limiting example, the viral genome comprises two signal sequence regions and the two signal sequence regions are: SIGNALS (SEQ ID NO: 13560) and SIGNAL15 (SEQ ID NO: 13569). As a non-limiting example, the viral genome comprises two signal sequence regions and the two signal sequence regions are: SIGNAL6 (SEQ ID NO: 13560) and SIGNAL16 (SEQ ID NO: 13570). As a non-limiting example, the viral genome comprises two signal sequence regions and the two signal sequence regions are: SIGNAL7 (SEQ ID NO: 13561) and SIGNALS (SEQ ID NO: 13562). As a non-limiting example, the viral genome comprises two signal sequence regions and the two signal sequence regions are: SIGNAL7 (SEQ ID NO: 13561) and SIGNAL9 (SEQ ID NO: 13563). As a non-limiting example, the viral genome comprises two signal sequence regions and the two signal sequence regions are: SIGNAL7 (SEQ ID NO: 13561) and SIGNAL10 (SEQ ID NO: 13564). As a non-limiting example, the viral genome comprises two signal sequence regions and the two signal sequence regions are: SIGNAL7 (SEQ ID NO: 13561) and SIGNAL11 (SEQ ID NO: 13565). As a non-limiting example, the viral genome comprises two signal sequence regions and the two signal sequence regions are: SIGNAL7 (SEQ ID NO: 13561) and SIGNAL12 (SEQ ID NO: 13566). As a non-limiting example, the viral genome comprises two signal sequence regions and the two signal sequence regions are: SIGNAL7 (SEQ ID NO: 13561) and SIGNAL13 (SEQ ID NO: 13567). As a non-limiting example, the viral genome comprises two signal sequence regions and the two signal sequence regions are: SIGNAL7 (SEQ ID NO: 13561) and SIGNAL14 (SEQ ID NO: 13568). As a non-limiting example, the viral genome comprises two signal sequence regions and the two signal sequence regions are: SIGNAL7 (SEQ ID NO: 13561) and SIGNAL15 (SEQ ID NO: 13569). As a non-limiting example, the viral genome comprises two signal sequence regions and the two signal sequence regions are: SIGNAL7 (SEQ ID NO: 13561) and SIGNAL16 (SEQ ID NO: 13570). As a non-limiting example, the viral genome comprises two signal sequence regions and the two signal sequence regions are: SIGNAL8 (SEQ ID NO: 13562) and SIGNAL9 (SEQ ID NO: 13563). As a non-limiting example, the viral genome comprises two signal sequence regions and the two signal sequence regions are: SIGNALS (SEQ ID NO: 13562) and SIGNAL11 (SEQ ID NO: 13564). As a non-limiting example, the viral genome comprises two signal sequence regions and the two signal sequence regions are: SIGNAL8 (SEQ ID NO: 13562) and SIGNAL11 (SEQ ID NO: 13565). As a non-limiting example, the viral genome comprises two signal sequence regions and the two signal sequence regions are: SIGNALS (SEQ ID NO: 13562) and SIGNAL12 (SEQ ID NO: 13566). As a non-limiting example, the viral genome comprises two signal sequence regions and the two signal sequence regions are: SIGNALS (SEQ ID NO: 13562) and SIGNAL13 (SEQ ID NO: 13567). As a non-limiting example, the viral genome comprises two signal sequence regions and the two signal sequence regions are: SIGNALS (SEQ ID NO: 13562) and SIGNAL14 (SEQ ID NO: 13568). As a non-limiting example, the viral genome comprises two signal sequence regions and the two signal sequence regions are: SIGNALS (SEQ ID NO: 13562) and SIGNAL15 (SEQ ID NO: 13569). As a non-limiting example, the viral genome comprises two signal sequence regions and the two signal sequence regions are: SIGNALS (SEQ ID NO: 13562) and SIGNAL16 (SEQ ID NO: 13570). As a non-limiting example, the viral genome comprises two signal sequence regions and the two signal sequence regions are: SIGNAL9 (SEQ ID NO: 13563) and SIGNAL10 (SEQ ID NO: 13564). As a non-limiting example, the viral genome comprises two signal sequence regions and the two signal sequence regions are: SIGNAL9 (SEQ ID NO: 13563) and SIGNAL11 (SEQ ID NO: 13565). As a non-limiting example, the viral genome comprises two signal sequence regions and the two signal sequence regions are: SIGNAL9 (SEQ ID NO: 13563) and SIGNAL12 (SEQ ID NO: 13566). As a non-limiting example, the viral genome comprises two signal sequence regions and the two signal sequence regions are: SIGNAL9 (SEQ ID NO: 13563) and SIGNAL13 (SEQ ID NO: 13567). As a non-limiting example, the viral genome comprises two signal sequence regions and the two signal sequence regions are: SIGNAL9 (SEQ ID NO: 13563) and SIGNAL14 (SEQ ID NO: 13568). As a non-limiting example, the viral genome comprises two signal sequence regions and the two signal sequence regions are: SIGNAL9 (SEQ ID NO: 13563) and SIGNAL15 (SEQ ID NO: 13569). As a non-limiting example, the viral genome comprises two signal sequence regions and the two signal sequence regions are: SIGNAL9 (SEQ ID NO: 13563) and SIGNAL16 (SEQ ID NO: 13570). As a non-limiting example, the viral genome comprises two signal sequence regions and the two signal sequence regions are: SIGNAL10 (SEQ ID NO: 13564) and SIGNAL11 (SEQ ID NO: 13565). As a non-limiting example, the viral genome comprises two signal sequence regions and the two signal sequence regions are: SIGNAL10 (SEQ ID NO: 13564) and SIGNAL12 (SEQ ID NO: 13566). As a non-limiting example, the viral genome comprises two signal sequence regions and the two signal sequence regions are: SIGNAL10 (SEQ ID NO: 13564) and SIGNAL13 (SEQ ID NO: 13567). As a non-limiting example, the viral genome comprises two signal sequence regions and the two signal sequence regions are: SIGNAL10 (SEQ ID NO: 13564) and SIGNAL14 (SEQ ID NO: 13568). As a non-limiting example, the viral genome comprises two signal sequence regions and the two signal sequence regions are: SIGNAL10 (SEQ ID NO: 13564) and SIGNAL15 (SEQ ID NO: 13569). As a non-limiting example, the viral genome comprises two signal sequence regions and the two signal sequence regions are: SIGNAL10 (SEQ ID NO: 13564) and SIGNAL16 (SEQ ID NO: 13570). As a non-limiting example, the viral genome comprises two signal sequence regions and the two signal sequence regions are: SIGNAL11 (SEQ ID NO: 13565) and SIGNAL12 (SEQ ID NO: 13566). As a non-limiting example, the viral genome comprises two signal sequence regions and the two signal sequence regions are: SIGNAL11 (SEQ ID NO: 13565) and SIGNAL13 (SEQ ID NO: 13567). As a non-limiting example, the viral genome comprises two signal sequence regions and the two signal sequence regions are: SIGNAL11 (SEQ ID NO: 13565) and SIGNAL14 (SEQ ID NO: 13568). As a non-limiting example, the viral genome comprises two signal sequence regions and the two signal sequence regions are: SIGNAL11 (SEQ ID NO: 13565) and SIGNAL15 (SEQ ID NO: 13569). As a non-limiting example, the viral genome comprises two signal sequence regions and the two signal sequence regions are: SIGNAL11 (SEQ ID NO: 13565) and SIGNAL16 (SEQ ID NO: 13570). As a non-limiting example, the viral genome comprises two signal sequence regions and the two signal sequence regions are: SIGNAL12 (SEQ ID NO: 13566) and SIGNAL13 (SEQ ID NO: 13567). As a non-limiting example, the viral genome comprises two signal sequence regions and the two signal sequence regions are: SIGNAL12 (SEQ ID NO: 13566) and SIGNAL14 (SEQ ID NO: 13568). As a non-limiting example, the viral genome comprises two signal sequence regions and the two signal sequence regions are: SIGNAL12 (SEQ ID NO: 13566) and SIGNAL15 (SEQ ID NO: 13569). As a non-limiting example, the viral genome comprises two signal sequence regions and the two signal sequence regions are: SIGNAL12 (SEQ ID NO: 13566) and SIGNAL16 (SEQ ID NO: 13570). As a non-limiting example, the viral genome comprises two signal sequence regions and the two signal sequence regions are: SIGNAL13 (SEQ ID NO: 13567) and SIGNAL14 (SEQ ID NO: 13568). As a non-limiting example, the viral genome comprises two signal sequence regions and the two signal sequence regions are: SIGNAL13 (SEQ ID NO: 13567) and SIGNAL15 (SEQ ID NO: 13569). As a non-limiting example, the viral genome comprises two signal sequence regions and the two signal sequence regions are: SIGNAL13 (SEQ ID NO: 13567) and SIGNAL16 (SEQ ID NO: 13570). As a non-limiting example, the viral genome comprises two signal sequence regions and the two signal sequence regions are: SIGNAL14 (SEQ ID NO: 13568) and SIGNAL15 (SEQ ID NO: 13569). As a non-limiting example, the viral genome comprises two signal sequence regions and the two signal sequence regions are: SIGNAL14 (SEQ ID NO: 13568) and SIGNAL16 (SEQ ID NO: 13570). As a non-limiting example, the viral genome comprises two signal sequence regions and the two signal sequence regions are: SIGNAL15 (SEQ ID NO: 13569) and SIGNAL16 (SEQ ID NO: 13570).

### Viral Genome Cassette Sequence Regions: Payload Sequence

The payload region may be any antibody sequence known in the art or may be any of the antibody sequences (these antibodies are also referred to as “reference antibody”) described herein such as, but not limited to, those in Tables 3-16. The payload region may comprise a variable domain sequence region, a constant domain sequence region, or both a variable domain and constant domain sequence region. The sequence regions may be from the light chain, heavy chain or both the light and heavy chain sequences of the reference antibody or they may be fragments or variants of the reference antibody. As a non-limiting example, the viral genome cassette includes a payload region of the variable domain of the light chain of the reference antibody. As a non-limiting example, the viral genome cassette includes a payload region of the variable domain of the light chain of a reference antibody and a constant domain of the light chain of the reference antibody. As a non-limiting example, the viral genome cassette includes a payload region of the variable domain of the heavy chain of the reference antibody and a constant domain of the heavy chain of the reference antibody. As a non-limiting example, the viral genome cassette includes a payload region of the variable domain of the heavy chain of the reference antibody, the constant domain of the heavy chain of the reference antibody, the variable domain of the light chain of the reference antibody, the constant domain of the light chain of the reference antibody. As a non-limiting example, the viral genome cassette includes a payload region of the variable domain of the heavy chain of the reference antibody, the constant domain of the heavy chain of the reference antibody, and the variable domain of the light chain of the reference antibody. As a non-limiting example, the viral genome cassette includes a payload region of the variable domain of the heavy chain of the reference antibody, the variable domain of the light chain of the reference antibody, the constant domain of the light chain of the reference antibody.

The viral genomes may also include one or more linker regions to separate coding (e.g., payload) or non-coding regions. Non-limiting examples of linker sequences are shown in Table 2. As a non-limiting example, the viral genome comprises at least one linker and that linker is LINKER 1 (SEQ ID NO: 1724). As a non-limiting example, the viral genome comprises at least one linker and that linker is LINKER 2 (SEQ ID NO: 1725). As a non-limiting example, the viral genome comprises at least one linker and that linker is LINKER 3 (SEQ ID NO: 1726). As a non-limiting example, the viral genome comprises at least one linker and that linker is LINKER 4 (SEQ ID NO: 1727). As a non-limiting example, the viral genome comprises at least one linker and that linker is LINKER 5 (SEQ ID NO: 1728). As a non-limiting example, the viral genome comprises at least one linker and that linker is LINKER 6 (SEQ ID NO: 1729). As a non-limiting example, the viral genome comprises at least one linker and that linker is LINKER 7 (SEQ ID NO: 1730). As a non-limiting example, the viral genome comprises at least one linker and that linker is LINKER 8 (SEQ ID NO: 1731). As a non-limiting example, the viral genome comprises at least one linker and that linker is LINKER 9 (SEQ ID NO: 1732). As a non-limiting example, the viral genome comprises at least one linker and that linker is LINKER 10 (SEQ ID NO: 1733). As a non-limiting example, the viral genome comprises at least one linker and that linker is LINKER 11 (SEQ ID NO: 1734). As a non-limiting example, the viral genome comprises at least one linker and that linker is LINKER 12 (SEQ ID NO: 1735). As a non-limiting example, the viral genome comprises at least one linker and that linker is LINKER 13 (SEQ ID NO: 1736). As a non-limiting example, the viral genome comprises at least one linker and that linker is LINKER 14 (SEQ ID NO: 1737). As a non-limiting example, the viral genome comprises at least one linker and that linker is LINKER 15 (SEQ ID NO: 1738). As a non-limiting example, the viral genome comprises at least one linker and that linker is LINKER 16 (SEQ ID NO: 1739). As a non-limiting example, the viral genome comprises at least one linker and that linker is LINKER 17 (SEQ ID NO: 13151). As a non-limiting example, the viral genome comprises at least one linker and that linker is LINKER 18 (SEQ ID NO: 13152). As a non-limiting example, the viral genome comprises at least one linker and that linker is LINKER 19 (SEQ ID NO: 13153). As a non-limiting example, the viral genome comprises at least one linker and that linker is LINKER 20 (SEQ ID NO: 13154). As a non-limiting example, the viral genome comprises at least one linker and that linker is LINKER 21 (SEQ ID NO: 13155). As a non-limiting example, the viral genome comprises at least one linker and that linker is LINKER 22 (SEQ ID NO: 13156). As a non-limiting example, the viral genome comprises at least one linker and that linker is LINKER 23 (SEQ ID NO: 13157). As a non-limiting example, the viral genome comprises at least one linker and that linker is LINKER 24 (SEQ ID NO: 13158). As a non-limiting example, the viral genome comprises at least one linker and that linker is LINKER 25 (SEQ ID NO: 13159). As a non-limiting example, the viral genome comprises at least one linker and that linker is LINKER 26 (SEQ ID NO: 13160). As a non-limiting example, the viral genome comprises at least one linker and that linker is LINKER 27 (SEQ ID NO: 13161). As a non-limiting example, the viral genome comprises at least one linker and that linker is LINKER 28 (SEQ ID NO: 13162). In some cases, the linker may be a peptide linker that may be used to connect the polypeptides encoded by the payload region (e.g., light and heavy antibody chains). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome.

### Exemplary Viral Genome Cassettes

In some embodiments, the viral genome comprises at least one sequence region described in Tables 17-24. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 3′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 3′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, an intron sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a tag sequence region, a PolyA sequence region, and a 3′ITR sequence region. As a non-limiting example, the viral genome comprises a 5′ITR sequence region, at least one promoter sequence region, an intron sequence region, a PolyA sequence region, a filler sequence region, and a 3′ITR sequence region. As a non-limiting example, the viral genome comprises a 5′ITR sequence region, at least one promoter sequence region, an intron sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, at least one linker sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a second signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 3′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, at least one linker sequence region, a first signal sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, at least one linker sequence region, a tag sequence region, a PolyA sequence region, and a 3′ITR sequence region. As a non-limiting example, the viral genome comprises a 5′ITR sequence region, at least one promoter sequence region, at least one linker sequence region, a PolyA sequence region, a filler sequence region, and a 3′ITR sequence region. As a non-limiting example, the viral genome comprises a 5′ITR sequence region, at least one promoter sequence region, at least one linker sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a tag sequence region, a PolyA sequence region, and a 3′ITR sequence region. As a non-limiting example, the viral genome comprises a 5′ITR sequence region, at least one promoter sequence region, a first signal sequence region, a PolyA sequence region, a filler sequence region, and a 3′ITR sequence region. As a non-limiting example, the viral genome comprises a 5′ITR sequence region, at least one promoter sequence region, a first signal sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region. As a non-limiting example, the viral genome comprises a 5′ITR sequence region, at least one promoter sequence region, a tag sequence region, a PolyA sequence region, a filler sequence region, and a 3′ITR sequence region. As a non-limiting example, the viral genome comprises a 5′ITR sequence region, at least one promoter sequence region, a tag sequence region, a PolyA sequence region, and a 3′ITR sequence region. As a non-limiting example, the viral genome comprises a 5′ITR sequence region, at least one promoter sequence region, a PolyA sequence region, a filler sequence region, and a 3′ITR sequence region. As a non-limiting example, the viral genome comprises a 5′ITR sequence region, at least one promoter sequence region, a PolyA sequence region, and a 3′ITR sequence region.

In some embodiments, the viral genome comprises at least one sequence region described in Tables 17-24 and include at least one payload sequence region encoding an antibody or fragment thereof described in Tables 3-16. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first signal sequence region, a first payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a second constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, at least one linker sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first signal sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first exon sequence region, a first intron sequence region, a second intron sequence region, a second exon sequence region, a first payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a second constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 3′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, at least one linker sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second signal sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first signal sequence region, a first payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, at least one linker sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a first signal sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a second constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first signal sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, at least one linker sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first signal sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, an intron sequence region, a first payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, at least one linker sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second signal sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a second constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second signal sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, at least one linker sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second signal sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first signal sequence region, a first payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a first signal sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, at least one linker sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a first signal sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a second constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first signal sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, at least one linker sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first signal sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a second payload region comprising a variable heavy or light chain sequence, variant or fragment thereof, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a first constant region of the first payload region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a tag sequence region, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a tag sequence region, a PolyA sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a PolyA sequence region, a filler sequence region, and a 3′ITR sequence region. As a non-limiting example, the viral genome comprises a 5′ITR sequence region, at least one promoter sequence region, a first payload region comprising a variable heavy or light chain sequence, variant or fragment thereof, a PolyA sequence region, and a 3′ITR sequence region.

In some embodiments, the AAV particle viral genome may comprise any of the viral genome cassettes (VGC) shown in Tables 25-91. In Tables 25-91, possible locations in the VGCs for the variable and/or constant sequence regions of the payload region are also described.

TABLE 25
Viral Genome Cassettes Sequence Regions

| | VGC1 | | VGC2 | |
|---|---|---|---|---|
| | Region SEQ | Region | Region SEQ | Region |
| Sequence Regions | ID NO | length | ID NO | length |
| 5′ ITR | 13519 | 130 | 13519 | 130 |
| CAG promoter | 13523 | 1715 | 13523 | 1715 |
| CMVie | 13524 | 299 | 13524 | 299 |
| CBA min. | 13525 | 283 | 13525 | 283 |
| Signal | 13567 | 57 | 13567 | 57 |
| Variable and/or Constant | — | — | — | — |
| Linker | 1724 | 12 | 1725 | 12 |
| Linker | 1726 | 54 | 1727 | 75 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC1 and comprises a 5′ inverted terminal repeat (ITR) sequence region and a 3′ ITR sequence region, a CAG promoter comprising a CMVie region and a minimal CBA promoter region, an antibody heavy chain signal sequence, an antibody heavy chain variable region and heavy chain constant region, a furin cleavage site, a T2A linker, an antibody light chain signal sequence, an antibody light chain variable region and light chain constant region, and a polyadenylation sequence.

In some embodiments, the AAV particle genome is VGC2 and comprises a 5′ inverted terminal repeat (ITR) sequence region and a 3′ ITR sequence region, a CAG promoter comprising a CMVie region and a minimal CBA promoter region, an antibody heavy chain signal sequence, an antibody heavy chain variable region and heavy chain constant region, a furin cleavage site, an F2A linker, an antibody light chain signal sequence, an antibody light chain variable region and light chain constant region, and a polyadenylation sequence.

TABLE 26
Viral Genome Cassettes Sequence Regions

| | VGC3 | | VGC4 | |
|---|---|---|---|---|
| | Region SEQ | Region | Region SEQ | Region |
| Sequence Regions | ID NO | length | ID NO | length |
| 5′ ITR | 13519 | 130 | 13519 | 130 |
| CB promoter | 13526 | 266 | 13526 | 260 |
| Exon | 13535 | 134 | 13535 | 134 |
| Intron | 13540 | 32 | 13540 | 32 |
| Intron | 13542 | 347 | 13542 | 347 |
| Exon | 13538 | 53 | 13538 | 53 |
| Signal | 13567 | 57 | 13567 | 57 |
| Variable and/or Constant | — | — | — | — |
| Linker | 1725 | 12 | 1724 | 12 |
| Linker | 1727 | 75 | 1726 | 54 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC3 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody heavy chain signal sequence, an antibody heavy chain variable region and heavy chain constant region, a furin cleavage site, an F2A linker, an antibody light chain signal sequence, an antibody light chain variable region and light chain constant region, and a polyadenylation sequence.

In some embodiments, the AAV particle genome is VGC4 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody heavy chain signal sequence, an antibody heavy chain variable region and heavy chain constant region, a furin cleavage site, a T2A linker, an antibody light chain signal sequence, an antibody light chain variable region and light chain constant region, and a polyadenylation sequence.

TABLE 27
Viral Genome Cassettes Sequence Regions

| | VGC5 | | VGC6 | | VGC7 | | VGC8 | |
|---|---|---|---|---|---|---|---|---|
| | Region | | Region | | Region | | Region | |
| | SEQ ID | Region | SEQ ID | Region | SEQ ID | Region | SEQ ID | Region |
| Sequence Regions | NO | length | NO | length | NO | length | NO | length |
| 5′ ITR | 13519 | 130 | 13519 | 130 | 13519 | 130 | 13519 | 130 |
| Promoter | 13528 | 699 | 13528 | 699 | 13529 | 557 | 13529 | 557 |
| Exon | 13535 | 134 | 13535 | 134 | 13535 | 134 | 13535 | 134 |
| Intron | 13540 | 32 | 13540 | 32 | 13540 | 32 | 13540 | 32 |
| Intron | 13542 | 347 | 13542 | 347 | 13542 | 347 | 13542 | 347 |
| Exon | 13538 | 53 | 13538 | 53 | 13538 | 53 | 13538 | 53 |
| Signal | 13567 | 57 | 13567 | 57 | 13567 | 57 | 13567 | 57 |
| Variable and/or | — | — | — | — | — | — | — | — |
| Constant | | | | | | | | |
| Linker | 1725 | 12 | 1724 | 12 | 1725 | 12 | 1724 | 12 |
| Linker | 1727 | 75 | 1726 | 54 | 1727 | 75 | 1726 | 54 |
| Signal | 13569 | 57 | 13569 | 57 | 13569 | 57 | 13569 | 57 |
| Variable and/or | — | — | — | — | — | — | — | — |
| Constant | | | | | | | | |
| Constant | 1864 | 321 | 1864 | 321 | 1864 | 321 | 1864 | 321 |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC5 and comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an antibody heavy chain signal sequence, an antibody heavy chain variable region and heavy chain constant region, a furin cleavage site, an F2A linker, an antibody light chain signal sequence, an antibody light chain variable region and light chain constant region, and a polyadenylation sequence.

In some embodiments, the AAV particle genome is VGC6 and comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an antibody heavy chain signal sequence, an antibody heavy chain variable region and heavy chain constant region, a furin cleavage site, a T2A linker, an antibody light chain signal sequence, an antibody light chain variable region and light chain constant region, and a polyadenylation sequence.

In some embodiments, the AAV particle genome is VGC7 and comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an antibody heavy chain signal sequence, an antibody heavy chain variable region and heavy chain constant region, a furin cleavage site, an F2A linker, an antibody light chain signal sequence, an antibody light chain variable region and light chain constant region, and a polyadenylation sequence.

In some embodiments, the AAV particle genome is VGC8 and comprises a 5′ inverted terminal repeat (ITR) sequence region and a 3′ ITR sequence region, a synapsin promoter, an ie1 exon region, an ie1 intron 1 region, a human beta-globin intron region, a human beta-globin exon region, an antibody heavy chain signal sequence, an antibody heavy chain variable region and heavy chain constant region, a furin cleavage site, a T2A linker, an antibody light chain signal sequence, an antibody light chain variable region and light chain constant region, and a polyadenylation sequence.

TABLE 28
Viral Genome Cassettes Sequence Regions

| | VGC9 | | VGC10 | |
|---|---|---|---|---|
| | Region SEQ | Region | Region SEQ | Region |
| Sequence Regions | ID NO | length | ID NO | length |
| 5′ ITR | 13519 | 130 | 13519 | 130 |
| CAG promoter | 13523 | 1715 | 13523 | 1715 |
| CMVie | 13524 | 299 | 13524 | 299 |
| CBA min. | 13525 | 283 | 13525 | 283 |
| Signal | 13569 | 57 | 13569 | 57 |
| Variable and/or Constant | — | — | — | — |
| Linker | 1727 | 75 | 1726 | 54 |
| Signal | 13567 | 57 | 13567 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC9 and comprises a 5′ inverted terminal repeat (ITR) sequence region and a 3′ ITR sequence region, a CAG promoter comprising a CMVie region and a minimal CBA promoter region, an antibody light chain signal sequence, an antibody light chain variable region and light chain constant region, an F2A linker, an antibody heavy chain signal sequence, an antibody heavy chain variable region and heavy chain constant region, and a polyadenylation sequence.

In some embodiments, the AAV particle genome is VGC10 and comprises a 5′ inverted terminal repeat (ITR) sequence region and a 3′ITR sequence region, a CAG promoter comprising a CMVie region and a minimal CBA promoter region, an antibody light chain signal sequence, an antibody light chain variable region and light chain constant region, a T2A linker, an antibody heavy chain signal sequence, an antibody heavy chain variable region and heavy chain constant region, and a polyadenylation sequence.

TABLE 29
Viral Genome Cassettes Sequence Regions

| | VGC11 | | VGC12 | |
|---|---|---|---|---|
| | Region SEQ | Region | Region SEQ | Region |
| Sequence Regions | ID NO | length | ID NO | length |
| 5′ ITR | 13519 | 130 | 13519 | 130 |
| Promoter | 13526 | 260 | 13526 | 260 |
| Exon | 13535 | 134 | 13535 | 134 |
| Intron | 13540 | 32 | 13540 | 32 |
| Intron | 13542 | 347 | 13542 | 347 |
| Exon | 13538 | 53 | 13538 | 53 |
| Signal | 13569 | 57 | 13569 | 57 |
| Variable and/or Constant | — | — | — | — |
| Linker | 1727 | 75 | 1726 | 54 |
| Signal | 13567 | 57 | 13567 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC11 and comprises a 5′ inverted terminal repeat (ITR) sequence region and a 3′ITR sequence region, a CB promoter, an ie1 exon 1 region, an ie1 intron region, a human beta-globin intron region, a human beta-globin exon region, an antibody light chain signal sequence, an antibody light chain variable region and light chain constant region, an F2A linker, an antibody heavy chain signal sequence, an antibody heavy chain variable region and heavy chain constant region, and a polyadenylation sequence.

In some embodiments, the AAV particle genome is VGC12 and comprises a 5′ inverted terminal repeat (ITR) sequence region and a 3′ITR sequence region, a CB promoter, an ie1 exon 1 region, an ie1 intron 1 region, a human beta-globin intron region, a human beta-globin exon region, an antibody light chain signal sequence, an antibody light chain variable region and light chain constant region, an T2A linker, an antibody heavy chain signal sequence, an antibody heavy chain variable region and heavy chain constant region, and a polyadenylation sequence.

TABLE 30
Viral Genome Cassettes Sequence Regions

| | VGC13 | | VGC14 | | VGC15 | | VGC16 | |
|---|---|---|---|---|---|---|---|---|
| Sequence | Region | Region | Region | Region | Region | Region | Region | Region |
| Regions | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 |
| Promoter | 13528 | 699 | 13528 | 699 | 13529 | 557 | 13529 | 557 |
| Exon | 13535 | 134 | 13535 | 134 | 13535 | 134 | 13535 | 134 |
| Intron | 13540 | 32 | 13540 | 32 | 13540 | 32 | 13540 | 32 |
| Intron | 13542 | 347 | 13542 | 347 | 13542 | 347 | 13542 | 347 |
| Exon | 13538 | 53 | 13538 | 53 | 13538 | 53 | 13538 | 53 |
| Signal | 13569 | 57 | 13569 | 57 | 13569 | 57 | 13569 | 57 |
| Variable | — | — | — | — | — | — | — | — |
| and/or | | | | | | | | |
| Constant | | | | | | | | |
| Linker | 1727 | 75 | 1726 | 54 | 1727 | 75 | 1726 | 54 |
| Signal | 13567 | 57 | 13567 | 57 | 13567 | 57 | 13567 | 57 |
| Variable | — | — | — | — | — | — | — | — |
| and/or | | | | | | | | |
| Constant | | | | | | | | |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC13 and comprises a 5′ inverted terminal repeat (ITR) sequence region and a 3′ITR sequence region, a GFAP promoter, an ie1 exon 1 region, an ie1 intron 1 region, a human beta-globin intron region, a human beta-globin exon region, an antibody light chain signal sequence, an antibody light chain variable region and light chain constant region, an F2A linker, an antibody heavy chain signal sequence, an antibody heavy chain variable region and heavy chain constant region, and a polyadenylation sequence.

In some embodiments, the AAV particle genome is VGC14 and comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, an antibody light chain signal sequence, an antibody light chain variable region and light chain constant region, a T2A linker, an antibody heavy chain signal sequence, an antibody heavy chain variable region and heavy chain constant region, and a polyadenylation sequence.

In some embodiments, the AAV particle genome is VGC15 and comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an antibody light chain signal sequence, an antibody light chain variable region and light chain constant region, an F2A linker, an antibody heavy chain signal sequence, an antibody heavy chain variable region and heavy chain constant region, and a polyadenylation sequence.

In some embodiments, the AAV particle genome is VGC16 and comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, an antibody light chain signal sequence, an antibody light chain variable region and light chain constant region, a T2A linker, an antibody heavy chain signal sequence, an antibody heavy chain variable region and heavy chain constant region, and a polyadenylation sequence.

TABLE 31
Viral Genome Cassettes Sequence Regions

| | VGC17 | | VGC18 | | VGC19 | | VGC20 | | VGC21 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 | 13519 | 130 |
| Promoter | 13526 | 260 | 13526 | 260 | 13526 | 260 | 13526 | 260 | 13526 | 260 |
| Exon | 13535 | 134 | 13535 | 134 | 13535 | 134 | 13535 | 134 | 13535 | 134 |
| Intron | 13540 | 32 | 13540 | 32 | 13540 | 32 | 13540 | 32 | 13540 | 32 |
| Intron | 13542 | 347 | 13542 | 347 | 13542 | 347 | 13542 | 347 | 13542 | 347 |
| Exon | 13538 | 53 | 13538 | 53 | 13538 | 53 | 13538 | 53 | 13538 | 53 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 1726 | 54 | 1726 | 54 | 1726 | 54 | 1726 | 54 | 1726 | 54 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC17 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (Bioinformatics) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC18 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (EMBOSS) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC19 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (GeneInfinity) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC20 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (GregThatcher) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC21 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (IDT) antibody light chain variable region, alight chain constant region, a T2A linker, an antibody heavy chain signal sequence, a codon-optimized antibody heavy chain variable region, a heavy chain constant region, and a polyadenylation sequence.

TABLE 32
Viral Genome Cassettes Sequence Regions

| | VGC22 | | VGC23 | | VGC24 | | VGC25 | | VGC26 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 | 13519 | 130 |
| Promoter | 13526 | 260 | 13526 | 260 | 13526 | 260 | 13526 | 260 | 13526 | 260 |
| Exon | 13535 | 134 | 13535 | 134 | 13535 | 134 | 13535 | 134 | 13535 | 134 |
| Intron | 13540 | 32 | 13540 | 32 | 13540 | 32 | 13540 | 32 | 13540 | 32 |
| Intron | 13542 | 347 | 13542 | 347 | 13542 | 347 | 13542 | 347 | 13542 | 347 |
| Exon | 13538 | 53 | 13538 | 53 | 13538 | 53 | 13538 | 53 | 13538 | 53 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 1726 | 54 | 1726 | 54 | 1726 | 54 | 1726 | 54 | 1726 | 54 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC22 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (InSilico) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC23 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (MolBio) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC24 and comprises a 5′ inverted terminal repeat (ITR) sequence region and a 3′ITR sequence region, a CB promoter, an ie1 exon 1 region, an ie1 intron 1 region, a human beta-globin intron region, a human beta-globin exon region, an antibody light chain signal sequence, a codon-optimized (N2P) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC25 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (SnapGene) antibody light chain variable region, alight chain constant region, a T2A linker, an antibody heavy chain signal sequence, a codon-optimized antibody heavy chain variable region, a heavy chain constant region, and a polyadenylation sequence.

In some embodiments, the AAV particle genome is VGC26 and comprises a 5′ inverted terminal repeat (ITR) sequence region and a 3′ ITR sequence region, a CB promoter, an ie1 exon 1 region, an ie1 intron 1 region, a human beta-globin intron region, a human beta-globin exon region, an antibody light chain signal sequence, a codon-optimized (Vector NTI) antibody light chain variable region, alight chain constant region, a T2A linker, an antibody heavy chain signal sequence, a codon-optimized antibody heavy chain variable region, a heavy chain constant region, and a polyadenylation sequence.

TABLE 33
Viral Genome Cassettes Sequence Regions

| | VGC27 | | VGC28 | | VGC29 | | VGC30 | | VGC31 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| Promoter | 13527 | 654 | 13527 | 654 | 13527 | 654 | 13527 | 654 | 13527 | 654 |
| Exon | 13535 | 134 | 13535 | 134 | 13535 | 134 | 13535 | 134 | 13535 | 134 |
| Intron | 13540 | 32 | 13540 | 32 | 13540 | 32 | 13540 | 32 | 13540 | 32 |
| Intron | 13542 | 347 | 13542 | 347 | 13542 | 347 | 13542 | 347 | 13542 | 347 |
| Exon | 13538 | 53 | 13538 | 53 | 13538 | 53 | 13538 | 53 | 13538 | 53 |
| Signal | 13567 | 57 | — | — | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 1724 | 12 | 1724 | 12 | 1724 | 12 | 1724 | 12 | 1724 | 12 |
| Linker | 1726 | 54 | 1726 | 54 | 1726 | 54 | 1726 | 54 | 1726 | 54 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| PolyA | 13577 | 477 | 13577 | 477 | 13577 | 477 | 13577 | 477 | 13577 | 477 |
| 3′ ITR | 13522 | 141 | 13522 | 141 | 13522 | 141 | 13522 | 141 | 13522 | 141 |

In some embodiments, the AAV particle genome is VGC27 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (GeneScript) antibody heavy chain variable region, a heavy chain constant region, a furin cleavage site, a T2A linker, an antibody light chain signal sequence, a codon-optimized antibody light chain variable region, a light chain constant region, and a polyadenylation sequence.

In some embodiments, the AAV particle genome is VGC28 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, a codon-optimized (SnapGene) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

In some embodiments, the AAV particle genome is VGC29 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (EMBOSS) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

In some embodiments, the AAV particle genome is VGC30 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (Bioinformatics) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

In some embodiments, the AAV particle genome is VGC31 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (NUS) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

TABLE 34
Viral Genome Cassettes Sequence Regions

| | VGC32 | | VGC33 | | VGC34 | | VGC35 | | VGC36 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| Promoter | 13527 | 654 | 13527 | 654 | 13527 | 654 | 13527 | 654 | 13527 | 654 |
| Exon | 13535 | 134 | 13535 | 134 | 13535 | 134 | 13535 | 134 | 13535 | 134 |
| Intron | 13540 | 32 | 13540 | 32 | 13540 | 32 | 13540 | 32 | 13540 | 32 |
| Intron | 13542 | 347 | 13542 | 347 | 13542 | 347 | 13542 | 347 | 13542 | 347 |
| Exon | 13538 | 53 | 13538 | 53 | 13538 | 53 | 13538 | 53 | 13538 | 53 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 1724 | 12 | 1724 | 12 | 1724 | 12 | 1724 | 12 | 1724 | 12 |
| Linker | 1726 | 54 | 1726 | 54 | 1726 | 54 | 1726 | 54 | 1726 | 54 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| PolyA | 13577 | 477 | 13577 | 477 | 13577 | 477 | 13577 | 477 | 13577 | 477 |
| 3′ ITR | 13522 | 141 | 13522 | 141 | 13522 | 141 | 13522 | 141 | 13522 | 141 |

In some embodiments, the AAV particle genome is VGC32 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (NUS2) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

In some embodiments, the AAV particle genome is VGC33 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, a codon-optimized (GeneInfinity) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

In some embodiments, the AAV particle genome is VGC34 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (IDT) antibody heavy chain variable region, a heavy chain constant region, aurin cleavage site, T2A linker, an antibody light chain signal sequence, a codon-optimized antibody light chain variable region, alight chain constant region, and a polyadenylation sequence.

In some embodiments, the AAV particle genome is VGC35 and comprises a 5′ inverted terminal repeat (ITR) sequence region and a 3′ITR sequence region, a CBA promoter, an ie1 exon 1 region, an ie1 intron region, a human beta-globin intron region, a human beta-globin exon region, an antibody heavy chain signal sequence, a codon-optimized (Bioinformatics 2) antibody heavy chain variable region, a heavy chain constant region, a furin cleavage site, a T2A linker, an antibody light chain signal sequence, a codon-optimized antibody light chain variable region, a light chain constant region, and a polyadenylation sequence.

In some embodiments, the AAV particle genome is VGC36 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, a codon-optimized (NUS3) antibody heavy chain variable region, a heavy chain constant region, a furin cleavage site, a T2A linker, an antibody light chain signal sequence, a codon-optimized antibody light chain variable region, alight chain constant region, and a polyadenylation sequence.

TABLE 35
Viral Genome Cassettes Sequence Regions

| | VGC37 | | VGC38 | | VGC39 | | VGC40 | | VGC41 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| CAG promoter | 13523 | 1715 | 13523 | 1715 | 13523 | 1715 | 13523 | 1715 | 13523 | 1715 |
| CMVie | 13524 | 299 | 13524 | 299 | 13524 | 299 | 13524 | 299 | 13524 | 299 |
| CBA min. | 13525 | 283 | 13525 | 283 | 13525 | 283 | 13525 | 283 | 13525 | 283 |
| Signal | 13568 | 57 | 13555 | 84 | 13555 | 84 | 13568 | 57 | 13568 | 57 |
| Variable | — | — | — | — | — | — | — | — | — | — |
| and/or | | | | | | | | | | |
| Constant | | | | | | | | | | |
| Linker | 1730 | 45 | 1730 | 45 | 1730 | 45 | 1730 | 45 | 1730 | 45 |
| Variable | — | — | — | — | — | — | — | — | — | — |
| and/or | | | | | | | | | | |
| Constant | | | | | | | | | | |
| Tag | 13571 | 27 | 13572 | 21 | 13574 | 18 | 13571 | 27 | 13574 | 18 |
| PolyA | 13576 | 127 | 13577 | 477 | 13577 | 477 | 13577 | 477 | 13577 | 477 |
| Filler | 13579 | 1153 | 13580 | 1240 | 13580 | 1240 | 13580 | 1240 | 13580 | 1240 |
| 3′ ITR | 13522 | 141 | 13522 | 141 | 13522 | 141 | 13522 | 141 | 13522 | 141 |

In some embodiments, the AAV particle genome is VGC37 and comprises a 5′ inverted terminal repeat (ITR) sequence region and a 3′ITR sequence region, a CAG promoter comprising a CMVie region and a minimal CBA promoter region, an antibody heavy chain signal sequence, an antibody heavy chain variable region, a (G4S)3 linker (“˜(M)3” disclosed as SEQ ID NO: 13143), an antibody light chain variable region, an HA tag, a polyadenylation signal sequence and a filler sequence.

In some embodiments, the AAV particle genome is VGC38 and comprises a 5′ inverted terminal repeat (ITR) sequence region and a 3′ITR sequence region, a CAG promoter comprising a CMVie region and a minimal CBA promoter region, a human growth hormone-2 signal sequence, an antibody heavy chain variable region, a (G4)3 linker (“(G4S)3” disclosed as SEQ ID NO: 13143), an antibody light chain variable region, an SEKDEL tag (“SEKDEL” disclosed as SEQ ID NO: 13164), a polyadenylation signal sequence and a filler sequence.

In some embodiments, the AAV particle genome is VGC39 and comprises a 5′ inverted terminal repeat (ITR) sequence region and a 3′ ITR sequence region, a CAG promoter comprising a CMVie region and a minimal CBA promoter region, a human growth hormone-2 signal sequence, an antibody heavy chain variable region, a (G4S)3 linker (“(G4S)3” disclosed as SEQ ID NO: 13143), an antibody light chain variable region, a His tag, a polyadenylation signal sequence and a filler sequence.

In some embodiments, the AAV particle genome is VGC40 and comprises a 5′ Inverted terminal repeat (ITR) sequence region and a 3′ ITR sequence region, a CAG promoter comprising a CMVie region and a minimal CBA promoter region, an antibody heavy chain signal sequence, an antibody heavy chain variable region, a (G4S)3 linker (“(G4S)3” disclosed as SEQ ID NO: 13143), an antibody light chain variable region, an HA tag, polyadenylation signal sequence and a filler sequence.

In some embodiments, the AAV particle genome is VGC41 and comprises a 5′ inverted terminal repeat (ITR) sequence region and a 3′ITR sequence region, a CAG promoter comprising CMVie region and minimal promoter region, an antibody heavy chain signal sequence, an antibody heavy chain variable region, a (G4S)3 linker (“(M4)3” disclosed as SEQ ID NO: 13143), an antibody light chain variable region, a His tag, a polyadenylation signal sequence and a filler sequence.

TABLE 36
Viral Genome Cassettes Sequence Regions

| | VGC42 | | VGC43 | | VGC44 | | VGC45 | | VGC46 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| CAG promoter | 13523 | 1715 | 13523 | 1715 | 13523 | 1715 | 13523 | 1715 | 13523 | 1715 |
| CMVie | 13524 | 299 | 13524 | 299 | 13524 | 299 | 13524 | 299 | 13524 | 299 |
| CBA min. | 13525 | 283 | 13525 | 283 | 13525 | 283 | 13525 | 283 | 13525 | 283 |
| Signal | 2016 | 84 | 13555 | 84 | 1862 | 57 | 1862 | 57 | 1862 | 57 |
| Variable | — | — | — | — | — | — | — | — | — | — |
| and/or | | | | | | | | | | |
| Constant | | | | | | | | | | |
| Linker | 1730 | 45 | 1730 | 45 | 1730 | 45 | 1730 | 45 | 1730 | 45 |
| Variable | — | — | — | — | — | — | — | — | — | — |
| and/or | | | | | | | | | | |
| Constant | | | | | | | | | | |
| Tag | 13573 | 18 | 13574 | 18 | 13571 | 27 | 13574 | 18 | 13571 | 27 |
| PolyA | 13577 | 447 | 13577 | 477 | 13577 | 477 | 13577 | 477 | 13576 | 127 |
| Filler | 13580 | 1240 | 13580 | 1240 | 13580 | 1240 | 13580 | 1240 | 13579 | 1153 |
| 3′ ITR | 13522 | 141 | 13522 | 141 | 13522 | 141 | 13522 | 141 | 13522 | 141 |

In some embodiments, the AAV particle genome is VGC42 and comprises a 5′ inverted terminal repeat (ITR) sequence region and a 3′ITR sequence region, a CAG promoter comprising a CMVie region and a minimal CBA promoter region, a human growth hormone-2 signal sequence, an antibody light chain variable region, a (G4S)3 linker (“(M4)3” disclosed as SEQ ID NO: 1131143), an antibody heavy chain variable region, a SEKDEL tag (“SEKDEL” disclosed as SEQ ID NO: 13164), a polyadenylation signal sequence and filler sequence.

In some embodiments, the AAV particle genome is VGC43 and comprises a 5′ inverted terminal repeat (ITR) sequence region and a 3′ITR sequence region, a CAG promoter comprising a CMVie region and a minimal CBA promoter region, a human growth hormone-2 signal sequence, an antibody light chain variable region, a (G4S)3 linker (“(G4S)3” disclosed as SEQ ID NO: 13143), an antibody heavy chain variable region, a His tag, a polyadenylation signal sequence and a filler sequence.

In some embodiments, the AAV particle genome is VGC44 and comprises a 5′ inverted terminal repeat (ITR) sequence region and a 3′ITR sequence region, a CAG promoter comprising a CMVie region and a minimal CBA promoter region, an antibody light chain signal sequence, an antibody light chain variable region, a (G4S)3 linker (“(G4S)3” disclosed as SEQ ID NO: 13143), an antibody heavy chain variable region, an His tag, a polyadenylation signal sequence and a filler sequence.

In some embodiments, the AAV particle genome is VGC45 and comprises a 5′ inverted terminal repeat (ITR) sequence region and a 3′ ITR sequence region, a CAG promoter comprising a CMVie region and a minimal CBA promoter region, an antibody light chain signal sequence, an antibody light chain variable region, a (G4S)3 linker (“(G4S)3” disclosed as SEQ ID NO: 13143), an antibody heavy chain variable region, a His tag, a polyadenylation signal sequence and a filler sequence.

In some embodiments, the AAV particle genome is VGC4 and comprises a 5′ inverted terminal repeat (ITR) sequence region and a 3′ITR sequence region, a CAG promoter comprising a CMVie region and a minimal CBA promoter region, an antibody light chain signal sequence, an antibody light chain variable region, a (G4S)3 linker (“(M4)3” disclosed as SEQ ID NO: 13143), an antibody heavy chain variable region, an HA tag, a polyadenylation signal sequence and filler sequence.

TABLE 37
Viral Genome Cassettes Sequence Regions

| | VGC47 | | VGC48 | | VGC49 | | VGC56 | | VGC51 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| CBA promoter | 13527 | 654 | 13527 | 654 | 13527 | 654 | 13527 | 654 | 13527 | 654 |
| Exon | 13535 | 134 | 13537 | 59 | 13536 | 102 | 13535 | 134 | 13535 | 134 |
| Intron | 13540 | 32 | 13540 | 32 | 13541 | 15 | 13541 | 15 | 13540 | 32 |
| Intron | 13544 | 73 | 13543 | 168 | 2102 | 53 | 2102 | 53 | 13545 | 73 |
| Exon | 13538 | 53 | 13538 | 53 | 13538 | 53 | 13538 | 53 | 13538 | 53 |
| Signal | 13568 | 57 | 13568 | 57 | 13568 | 57 | 13568 | 57 | 13568 | 57 |
| Variable | — | — | — | — | — | — | — | — | — | — |
| and/or | | | | | | | | | | |
| Constant | | | | | | | | | | |
| Linker | 1730 | 45 | 1730 | 45 | 1730 | 45 | 1730 | 45 | 1730 | 45 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13522 | 141 | 13522 | 141 | 13522 | 141 | 13522 | 141 | 13522 | 141 |

In some embodiments, the AAV particle genome is VGC47 and comprises a 5′ inverted terminal repeat (ITR) sequence region and a 3′ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4S)3 linker (“(G4S)3” disclosed as SEQ ID NO: 13143), an antibody light chain variable region, an HA tag, and a polyadenylation signal sequence.

In some embodiments, the AAV particle genome is VGC48 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G48)3 linker (“(G4S)3” disclosed as SEQ ID NO: 13143), an antibody light chain variable region, an HA tag, and a polyadenylation signal sequence.

In some embodiments, the AAV particle genome is VGC49 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4S)3 linker (“(G4)3” disclosed as SEQ ID NO: 13143), an antibody light chain variable region, an HA tag, and a polyadenylation signal sequence.

In some embodiments, the AAV particle genome is VGC50 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4)3 linker (“(G4S)3” disclosed as SEQ ID NO: 13143), an antibody light chain variable region, an HA tag, and a polyadenylation signal sequence.

In some embodiments, the AAV particle genome is VGC51 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4S)3 linker (“(G4S)3” disclosed as SEQ ID NO: 13143), an antibody light chain variable region, an HA tag, and a polyadenylation signal sequence.

TABLE 38
Viral Genome Cassettes Sequence Regions

| | VGC52 | | VGC53 | | VGC54 | | VGC55 | | VGC56 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| CBA promoter | 13527 | 654 | 13527 | 654 | 13527 | 654 | 13527 | 654 | 13527 | 654 |
| Exon | 13537 | 59 | 13535 | 134 | 13535 | 134 | — | — | — | — |
| Intron | 13540 | 32 | 13540 | 32 | 13540 | 32 | — | — | 2103 | 172 |
| Intron | 13543 | 168 | 13542 | 347 | 13542 | 347 | — | — | — | — |
| Exon | 13538 | 53 | 13538 | 53 | 13538 | 53 | — | — | — | — |
| Signal | 1862 | 57 | 1862 | 57 | 1862 | 57 | 1862 | 57 | 1862 | 57 |
| Variable | — | — | — | — | — | — | — | — | — | — |
| and/or | | | | | | | | | | |
| Constant | | | | | | | | | | |
| Linker | 1730 | 45 | 1730 | 45 | 1730 | 45 | 1730 | 45 | 1730 | 45 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| Filler | — | — | — | — | 13579 | 1153 | — | — | — | — |
| 3′ ITR | 13522 | 141 | 13522 | 141 | 13522 | 141 | 13522 | 141 | 13522 | 141 |

In some embodiments, the AAV particle genome is VGC52 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody light chain signal sequence, an antibody light chain variable region, a (G4S)3 linker (“(G4)3” disclosed as SEQ ID NO: 13143), an antibody heavy chain variable region, an HA tag, and a polyadenylation signal sequence.

In some embodiments, the AAV particle genome is VGC53 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody light chain signal sequence, an antibody light chain variable region, a (G4S)3 linker (“(G4)3” disclosed as SEQ ID NO: 13143), an antibody heavy chain variable region, an HA tag, and a polyadenylation signal sequence.

In some embodiments, the AAV particle genome is VGC54 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody light chain signal sequence, an antibody light chain variable region, a (G4S)3 linker (“(G4S)3” disclosed as SEQ ID NO: 13143), an antibody heavy chain variable region, an HA tag, a polyadenylation signal sequence and a filler sequence.

In some embodiments, the AAV particle genome is VGC55 and comprises a 5′ inverted terminal repeat (ITR) sequence region and a 3′ ITR sequence region, a CBA promoter, an antibody light chain signal sequence, an antibody light chain variable region, a (G4S)3 linker (“(G4S)3” disclosed as SEQ ID NO: 13143), an antibody heavy chain variable region, an HA tag, and a polyadenylation signal sequence.

In some embodiments, the AAV particle genome is VGC56 and comprises a 5′ inverted terminal repeat (ITR) sequence region and a 3′ ITR sequence region, a CBA promoter, an SV40 intron, an antibody light chain signal sequence, an antibody light chain variable region, a (G4S)3 linker (“(G4S)3” disclosed as SEQ ID NO: 13143), an antibody heavy chain variable region, an HA tag, and a polyadenylation signal sequence.

TABLE 39
Viral Genome Cassettes Sequence Regions

| | VGC57 | | VGC58 | | VGC59 | | VGC60 | | VGC61 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13520 | 141 | 13520 | 141 | 13520 | 141 | 13520 | 141 | 13520 | 141 |
| CBA promoter | 13527 | 654 | 13527 | 654 | 13527 | 654 | 13527 | 654 | 13527 | 654 |
| Exon | 13535 | 134 | 13535 | 134 | 13535 | 134 | — | — | — | — |
| Intron | 13540 | 32 | 13540 | 32 | 13540 | 32 | — | — | 2103 | 172 |
| Intron | 13542 | 347 | 13542 | 347 | 2101 | 73 | — | — | — | — |
| Exon | 13538 | 53 | 13538 | 53 | 13538 | 53 | — | — | — | — |
| Signal | 13568 | 57 | 13568 | 57 | 13568 | 57 | 13568 | 57 | 13568 | 57 |
| Variable | — | — | — | — | — | — | — | — | — | — |
| and/or | | | | | | | | | | |
| Constant | | | | | | | | | | |
| Linker | 1730 | 45 | 1730 | 45 | 1730 | 45 | 1736 | 45 | 1730 | 45 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| Filler | 13579 | 1153 | — | — | — | — | — | — | — | — |
| 3′ ITR | 13522 | 141 | 13522 | 141 | 13522 | 141 | 13522 | 141 | 13522 | 141 |

In some embodiments, the AAV particle genome is an comprises a inverted terminal repeat sequence region and a 3′ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4S)3 linker (“(G4S)3” disclosed as SEQ ID NO: 13143), an antibody light chain variable region, an HA tag, a polyadenylation signal sequence and a filler sequence.

In some embodiments, the AAV particle genome is VGC58 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4S)3 linker (“(G48)3” disclosed as SEQ ID NO: 13143), an antibody light chain variable region, an HA tag, and a polyadenylation signal sequence.

In some embodiments, the AAV particle genome is VGC59 and comprises a 5′ inverted terminal repeat (ITR) sequence region and a 3′ ITR sequence region, a CBA promoter, an ie1 exon 1 region, an ie1 intron 1 region, a human beta-globin intron region, a human beta-globin exon region, an antibody heavy chain signal sequence, an antibody heavy chain variable region, a (G4S)3 linker (“(G4)3” disclosed as SEQ ID NO: 13143), an antibody light chain variable region, an HA tag, and a polyadenylation signal sequence.

In some embodiments, the AAV particle genome is VGC60 and comprises a 5′ inverted terminal repeat (ITR) sequence region and a 3′ ITR sequence region, a CBA promoter, an antibody heavy chain signal sequence, an antibody heavy chain variable region, a (G4S)3 linker (“(G4)3” disclosed as SEQ ID NO: 13143), an antibody light chain variable region, an HA tag, and a polyadenylation signal sequence.

In some embodiments, the AAV particle genome is VGC61 and comprises a 5′ inverted terminal repeat (ITR) sequence region and a 3′ ITR sequence region, a CBA promoter, an SV40 intron, an antibody heavy chain signal sequence, an antibody heavy chain variable region, a (G4S)3 linker (“(G4S)3” disclosed as SEQ ID NO: 13143), an antibody light chain variable region, an HA tag, and a polyadenylation signal sequence.

TABLE 40
Viral Genome Cassettes Sequence Regions

| | VGC62 | | VGC63 | | VGC64 | | VGC65 | | VGC66 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 | 13519 | 130 |
| CMVie | 13530 | 382 | 13530 | 382 | 13530 | 382 | 13530 | 382 | 13530 | 382 |
| Promoter | 13526 | 260 | 13526 | 260 | 13526 | 260 | 13526 | 260 | 13526 | 260 |
| Intron | 2103 | 172 | 2103 | 172 | 2103 | 172 | 2103 | 172 | 2103 | 172 |
| Variable | — | — | — | — | — | — | — | — | — | — |
| and/or | | | | | | | | | | |
| Constant | | | | | | | | | | |
| Linker | 1725 | 12 | 1725 | 12 | — | — | — | — | — | — |
| Linker | 1727 | 75 | 1728 | 66 | 1727 | 75 | 1732 | 609 | 1728 | 66 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC62 and comprises a 5′ inverted terminal repeat (ITR) sequence region and a 3′ ITR sequence region, a CMVie region, a CB promoter, an SV40 intron, an antibody heavy chain variable region, a furin cleavage site, an F2A linker, an antibody light chain variable region, and a polyadenylation signal sequence.

In some embodiments, the AAV particle genome is VGC63 and comprises a 5′ inverted terminal repeat (ITR) sequence region and a 3′ ITR sequence region, a CMVie region, a CB promoter, an SV40 intron, an antibody heavy chain variable region, a furin cleavage site, a P2A linker, an antibody light chain variable region, and a polyadenylation signal sequence.

In some embodiments, the AAV particle genome is VGC64 and comprises a 5′ inverted terminal repeat (ITR) sequence region and a 3′ ITR sequence region, a CMVie region, a CB promoter, an SV40 intron, an antibody heavy chain variable region, an F2A linker, an antibody light chain variable region, and a polyadenylation signal sequence.

In some embodiments, the AAV particle genome is VGC65 and comprises a 5′ inverted terminal repeat (ITR) sequence region and a 3′ ITR sequence region, a CMVie region, a CB promoter, an SV40 intron, an antibody heavy chain variable region, an IRES linker, an antibody light chain variable region, and a polyadenylation signal sequence.

In some embodiments, the AAV particle genome is VGC66 and comprises a 5′ inverted terminal repeat (ITR) sequence region and a 3′ ITR sequence region, a CMVie region, a CB promoter, an SV40 intron, an antibody heavy chain variable region, a P2A linker, an antibody light chain variable region, and a polyadenylation signal sequence.

TABLE 41
Viral Genome Cassettes Sequence Regions

| | VGC67 | | VGC68 | |
|---|---|---|---|---|
| | Region SEQ | Region SEQ | Region | Region |
| Sequence Regions | ID NO | ID NO | length | length |
| 5′ ITR | 13519 | 130 | 13519 | 130 |
| CMVie | 13530 | 382 | 13530 | 382 |
| Promoter | 13526 | 260 | 13526 | 260 |
| Intron | 2103 | 172 | 2103 | 172 |
| Variable and/or Constant | — | — | — | — |
| Linker | 1725 | 12 | 1725 | 12 |
| Linker | 1727 | 75 | 1727 | 75 |
| Variable and/or Constant | — | — | — | — |
| PolyA | 13576 | 127 | 13576 | 127 |
| 3′ ITR | 13521 | 130 | 13521 | 130 |

In some embodiments, the MV particle genome is VGC67 and comprises a 5′ inverted terminal repeat (ITR) sequence region and a 3′ITR sequence region, a CMVie region, a CB promoter, an SV40 intron an antibody heavy chain variable region, a furin cleavage site, an F2A linker, an antibody light chain variable region, and polyadenylation signal sequence.

In some embodiments, the AAV particle genome is VGC68 and comprises a 5′ inverted terminal repeat (ITR) sequence region and a 3′ ITR sequence region, a CMVie region, a CB promoter, an SV40 intron, an antibody heavy chain variable region, a furin cleavage site, an F2A linker, an antibody light chain variable region, and a polyadenylation signal sequence.

TABLE 42
Viral Genome Cassettes Sequence Regions

| | VGC69 | | VGC70 | | VGC71 | | VGC72 | | VGC73 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 | 13519 | 130 |
| CMVie | 13530 | 382 | 13530 | 382 | 13530 | 382 | 13530 | 382 | 13530 | 382 |
| Promoter | 13526 | 260 | 13526 | 260 | 13526 | 260 | 13526 | 260 | 13526 | 260 |
| SV40 | 2103 | 172 | 2103 | 172 | 2103 | 172 | 2103 | 172 | 2103 | 172 |
| Variable | — | — | — | — | — | — | — | — | — | — |
| and/or | | | | | | | | | | |
| Constant | | | | | | | | | | |
| Linker | 1725 | 12 | — | — | — | — | — | — | — | — |
| Linker | 1727 | 75 | 1727 | 75 | 1732 | 609 | 1724 | 75 | 1728 | 66 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC69 and comprises a 5′ inverted terminal repeat (ITR) sequence region and a 3′ ITR sequence region, a CMVie region, a CB promoter, an SV40 intron, an antibody light chain variable region, a furin cleavage site, an F2A linker, an antibody heavy chain variable region, and a polyadenylation signal sequence.

In some embodiments, the AAV particle genome is VGC70 and comprises a 5′ inverted terminal repeat (ITR) sequence region and a 3′ ITR sequence region, a CMVie region, a CB promoter, an 5V40 intron, an antibody light chain variable region, an F2A linker, an antibody heavy chain variable region, and a polyadenylation signal sequence.

In some embodiments, the AAV particle genome is VGC71 and comprises a 5′ inverted terminal repeat (ITR) sequence region and a 3′ITR sequence region, a CMVie region, a CB promoter, an SV40 intron, an antibody light chain variable region, an IRES linker, an antibody heavy chain variable region, and a polyadenylation signal sequence.

In some embodiments, the AAV particle genome is VGC72 and comprises a 5′ inverted terminal repeat (ITR) sequence region and a 3′ ITR sequence region, a CMVie region, a CB promoter, an SV40 intron, an antibody light chain variable region, a (G4S)5 linker (“(G4S)5” disclosed as SEQ ID NO: 13144), an antibody heavy chain variable region, and a polyadenylation signal sequence.

In some embodiments, the AAV particle genome is VGC73 and comprises a 5′ inverted terminal repeat (ITR) sequence region and a 3′ITR sequence region, a CMVie region, a CB promoter, an SV40 intron, an antibody light chain variable region, an P2A linker, an antibody heavy chain variable region, and a polyadenylation signal sequence.

TABLE 43
Viral Genome Cassettes Sequence Regions

| | VGC74 | | VGC75 | | VGC76 | |
|---|---|---|---|---|---|---|
| Sequence | Region | Region | Region | Region | Region | Region |
| Regions | SEQ ID NO | length | SEQ ID NO | length | SEQ ID NO | length |
| 5′ ITR | 13519 | 130 | 13519 | 130 | 13519 | 130 |
| CMVie | 13530 | 382 | 13530 | 382 | 13530 | 382 |
| Promoter | 13526 | 260 | 13526 | 260 | 13526 | 260 |
| Intron | 2103 | 172 | 2103 | 172 | 2103 | 172 |
| Variable | — | — | — | — | — | — |
| and/or | | | | | | |
| Constant | | | | | | |
| Linker | 1725 | 12 | — | — | — | — |
| Linker | 1728 | 66 | 1727 | 75 | 1727 | 75 |
| Variable | — | — | — | — | — | — |
| and/or | | | | | | |
| Constant | | | | | | |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC74 and comprises a 5′ inverted terminal repeat (ITR) sequence region and a 3′ ITR sequence region, a CMVie region, a CB promoter, an SV40 intron, an antibody light chain variable region, a furin cleavage site, a P2A linker, an antibody heavy chain variable region, and a polyadenylation signal sequence.

In some embodiments, the AAV particle genome is VGC75 and comprises a 5′ inverted terminal repeat (ITR) sequence region and a 3′ ITR sequence region, a CMVie region, a CB promoter, an SV40 intron, an antibody light chain variable region, an F2A linker, an antibody heavy chain variable region, and a polyadenylation signal sequence.

In some embodiments, the AAV particle genome is VGC76 and comprises a 5′ inverted terminal repeat (ITR) sequence region and a 3′ ITR sequence region, a CMVie region, a CB promoter, an SV40 intron, an antibody light chain variable region, an F2A linker, an antibody heavy chain variable region, and a polyadenylation signal sequence.

TABLE 44
Viral Genome Cassettes Sequence Regions

| | VGC77 | | VGC78 | | VGC79 | | VGC80 | |
|---|---|---|---|---|---|---|---|---|
| Sequence | Region | Region | Region | Region | Region | Region | Region | Region |
| Regions | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 |
| CAG promoter | 13523 | 1715 | 13523 | 1715 | 13523 | 1715 | 13523 | 1715 |
| CMVie | 13524 | 299 | 13524 | 299 | 13524 | 299 | 13524 | 299 |
| CBA min. | 13525 | 283 | 13525 | 283 | 13525 | 283 | 13525 | 283 |
| Signal | 13567 | 57 | 13567 | 57 | 13567 | 57 | 13567 | 57 |
| Variable | — | — | — | — | — | — | — | — |
| and/or | | | | | | | | |
| Constant | | | | | | | | |
| Linker | 1725 | 12 | 1724 | 12 | 1724 | 12 | 1724 | 12 |
| Linker | 1727 | 75 | 1726 | 54 | 1726 | 54 | 1726 | 54 |
| Signal | 13569 | 57 | 13569 | 57 | 13569 | 57 | 13569 | 57 |
| Variable | — | — | — | — | — | — | — | — |
| and/or | | | | | | | | |
| Constant | | | | | | | | |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC77 and comprises a 5′ inverted terminal repeat (ITR) sequence region and a 3′ ITR sequence region, a CAG promoter comprising a CMVie region and a minimal CBA promoter region, a heavy chain signal sequence, an antibody heavy chain variable region, a heavy chain constant region, a furin cleavage site, a F2A linker, alight chain signal sequence, an antibody light chain variable region, alight chain constant region, and a polyadenylation signal sequence.

In some embodiments, the AAV particle genome is VGC78 and comprises a 5′ inverted terminal repeat (ITR) sequence region and a 3′ ITR sequence region, a CAG promoter comprising a CMVie region and a minimal CBA promoter region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC79 and comprises a 5′ inverted terminal repeat (ITR) sequence region and a 3′ ITR sequence region, a CAG promoter comprising a CMVie region and a minimal CBA promoter region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC80 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

TABLE 45
Viral Genome Cassettes Sequence Regions

| | VGC81 | | VGC82 | |
|---|---|---|---|---|
| | Region SEQ | Region | Region SEQ | Region |
| Sequence Regions | ID NO | length | ID NO | length |
| 5′ ITR | 13519 | 130 | 13519 | 130 |
| CAG promoter | 13523 | 1715 | 13523 | 1715 |
| CMVie | 13524 | 299 | 13524 | 299 |
| CBA min. | 13525 | 283 | 13525 | 283 |
| Signal | 13567 | 57 | 13567 | 57 |
| Variable and/or Constant | — | — | — | — |
| Linker | 13151 | 198 | 13151 | 198 |
| Linker | 1724 | 12 | 1724 | 12 |
| Linker | 1726 | 54 | 1726 | 54 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC81 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a heavy chain constant region, a hinge region, a furin cleavage site, a T2A linker, a light chain signal sequence, an antibody light chain variable region, a light chain constant region, and a polyadenylation signal sequence.

In some embodiments, the AAV particle genome is VGC82 and comprises a 5′ inverted terminal repeat (ITR) sequence region and a 3′ITR sequence region, a CAG promoter comprising a CMVie region and minimal CBA promoter region, heavy chain signal sequence, an antibody heavy chain variable region, a heavy chain constant region, a hinge region, a furin cleavage site, a T2A linker, a light chain signal sequence, an antibody light chain variable region, a light chain constant region, and a polyadenylation signal sequence.

TABLE 46
Viral Genome Cassettes Sequence Regions

| | VGC83 | | VGC84 | | VGC85 | | VGC86 | | VGC87 | |
|---|---|---|---|---|---|---|---|---|---|---|
| Sequence | Region | Region | Region | Region | Region | Region | Region | Region | Region | Region |
| Regions | SEQ ID NO | length | SEQ ID NO | length | SEQ ID NO | length | SEQ ID NO | length | SEQ ID NO | length |
| 5′ ITR | 13519 | 130 | 13519 | 130 | 13519 | 130 | 13519 | 130 | 13519 | 130 |
| CAG promoter | 13523 | 1715 | 13523 | 1715 | 13523 | 1715 | 13523 | 1715 | 13523 | 1715 |
| CMVie | 13524 | 299 | 13524 | 299 | 13524 | 299 | 13524 | 299 | 13524 | 299 |
| CBA min. | 13525 | 283 | 13525 | 283 | 13525 | 283 | 13525 | 283 | 13525 | 283 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 13154 | 45 | 13157 | 75 | 13160 | 120 | 13152 | 15 | 13153 | 30 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC83 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a (G4S)3 linker (“(G4S)3” disclosed as SEQ ID NO: 13143), an antibody heavy chain variable region, an HA tag, and a polyadenylation signal sequence.

In some embodiments, the AAV particle genome is VGC84 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a (G4S)3 linker (“(G4)3” disclosed as SEQ ID NO: 13143), an antibody heavy chain variable region, an HA tag, and a polyadenylation signal sequence.

In some embodiments, the AAV particle genome is VGC85 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a (G4S)8 linker (“(G4)8” disclosed as SEQ ID NO: 13148), an antibody heavy chain variable region, an HA tag, and a polyadenylation signal sequence.

In some embodiments, the AAV particle genome is VGC86 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a G4S linker (“G4S” disclosed as SEQ ID NO: 13141), an antibody heavy chain variable region, an HA tag, and a polyadenylation signal sequence.

In some embodiments, the AAV particle genome is VGC87 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a (G4S)2 linker (“(G4)2” disclosed as SEQ ID NO: 13145), an antibody heavy chain variable region, an HA tag, and a polyadenylation signal sequence.

TABLE 47
Viral Genome Cassettes Sequence Regions

| | VGC88 | | VGC89 | | VGC90 | | VGC91 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 130 | 13519 | 130 | 13519 | 130 |
| CAG promoter | 13523 | 1715 | 13523 | 1715 | 13523 | 1715 | 13523 | 1715 |
| CMVie | 13524 | 299 | 13524 | 299 | 13524 | 299 | 13524 | 299 |
| CBA min. | 13525 | 283 | 13525 | 283 | 13525 | 283 | 13525 | 283 |
| Signal | 13569 | 57 | 13569 | 57 | 13569 | 57 | 13569 | 57 |
| Variable | — | — | — | — | — | — | — | — |
| and/or | | | | | | | | |
| Constant | | | | | | | | |
| Linker | 1727 | 75 | 1726 | 54 | 1726 | 54 | 1726 | 54 |
| Signal | 13567 | 57 | 13567 | 57 | 13567 | 57 | 13567 | 57 |
| Variable | — | — | — | — | — | — | — | — |
| and/or | | | | | | | | |
| Constant | | | | | | | | |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC88 and comprises a 5′ inverted terminal repeat (ITR) sequence region and a 3′ ITR sequence region, a CAG promoter comprising CMV region and a minimal CBA promoter region, light chain signal sequence, an antibody light chain variable region, a light chain constant region a F2A linker a heavy chain signal sequence, an antibody heavy chain variable region, a heavy chain constant region and a polyadenylation signal sequence.

In some embodiments, the AAV particle genome is VGC89 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a light chain constant region, a T2A linker, a heavy chain signal sequence, an antibody heavy chain variable region, a heavy chain constant region, and a polyadenylation signal sequence.

In some embodiments, the AAV particle genome is VGC90 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, alight chain constant region, a T2A linker, a heavy chain signal sequence, an antibody heavy chain variable region, a heavy chain constant region, and a polyadenylation signal sequence.

In some embodiments, the AAV particle genome is VGC91 and comprises a 5′ inverted terminal repeat (ITR) sequence region and a 3′ITR sequence region, a CAG promoter comprising a CMVie region and a minimal CBA promoter region, a light chain signal sequence, an antibody light chain variable region, alight chain constant region, a T2A linker, a heavy chain signal sequence, an antibody heavy chain variable region, a heavy chain constant region, and a polyadenylation signal sequence.

TABLE 48
Viral Genome Cassettes Sequence Regions

| | VGC92 | | VGC93 | | VGC94 | | VGC95 | | VGC96 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 136 | 13519 | 130 | 13519 | 130 | 13519 | 130 |
| Promoter | 13528 | 699 | 13528 | 699 | 13528 | 699 | 13528 | 699 | 13528 | 699 |
| Intron | 13551 | 566 | 13551 | 566 | 13551 | 566 | 13551 | 566 | 13551 | 566 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 1725 | 12 | 1724 | 12 | 1724 | 12 | 1724 | 12 | 1724 | 12 |
| Linker | 1727 | 75 | 1726 | 54 | 1726 | 54 | 1726 | 54 | 1726 | 54 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC92 and comprises a 5′ inverted terminal repeat (ITR) sequence region and a 3′ITR sequence region, a GFAP promoter, a human beta-globin intron region, a heavy chain signal sequence, an antibody heavy chain variable region, a heavy chain constant region, a furin cleavage site, a F2A linker, a light chain signal sequence, an antibody light chain variable region, alight chain constant region, and a polyadenylation signal sequence.

In some embodiments, the AAV particle genome is VGC93 and comprises a 5′ inverted terminal repeat (ITR) sequence region and a 3′ ITR sequence region, a GFAP promoter, a human beta-globin intron region, a heavy chain signal sequence, an antibody heavy chain variable region, a heavy chain constant region, a furin cleavage site, a T2A linker, a light chain signal sequence, an antibody light chain variable region, alight chain constant region, and a polyadenylation signal sequence.

In some embodiments, the AAV particle genome is VGC94 and comprises a 5′ inverted terminal repeat (ITR) sequence region and a 3′ITR sequence region, a GFAP promoter, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC95 and comprises a 5′ inverted terminal repeat (ITR) sequence region and a 3′ ITR sequence region, a GFAP promoter, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC96 and comprises a 5′ inverted terminal repeat (ITR) sequence region and a 3′ ITR sequence region, a GFAP promoter, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

TABLE 49
Viral Genome Cassettes Sequence Regions

| | VGC97 | | VGC98 | | VGC99 | | VGC100 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 130 | 13519 | 130 | 13519 | 130 |
| Promoter | 13528 | 699 | 13528 | 699 | 13529 | 557 | 13534 | 380 |
| Intron | 13551 | 566 | 13551 | 566 | 13551 | 566 | 13551 | 566 |
| Signal | 13567 | 57 | 13567 | 57 | 13567 | 57 | 13570 | 57 |
| Variable | — | — | — | — | — | — | — | — |
| and/or | | | | | | | | |
| Constant | | | | | | | | |
| Linker | 13151 | 198 | 13151 | 198 | 13151 | 198 | 13151 | 198 |
| Linker | 1724 | 12 | 1724 | 12 | 1724 | 12 | 1724 | 12 |
| Linker | 1726 | 54 | 1726 | 54 | 1726 | 54 | 1726 | 54 |
| Signal | 13569 | 57 | 13569 | 57 | 13569 | 57 | 13569 | 57 |
| Variable | — | — | — | — | — | — | — | — |
| and/or | | | | | | | | |
| Constant | | | | | | | | |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC97 and comprises a 5′ inverted terminal repeat (ITR) sequence region and a 3′ ITR sequence region, a GFAP promoter, a human beta-globin intron region, a heavy chain signal sequence, an antibody heavy chain variable region, a heavy chain constant region, a hinge region, a furin cleavage site, a T2A linker, alight chain signal sequence, an antibody light chain variable region, alight chain constant region, and a polyadenylation signal sequence.

In some embodiments, the AAV particle genome is VGC98 and comprises a 5′ inverted terminal repeat (ITR) sequence region and a 3′ ITR sequence region, a GFAP promoter, a human beta-globin intron region, a heavy chain signal sequence, an antibody heavy chain variable region, a heavy chain constant region, a hinge region, a furin cleavage site, a T2A linker, alight chain signal sequence, an antibody light chain variable region, alight chain constant region, and a polyadenylation signal sequence.

In some embodiments, the AAV particle genome is VGC99 and comprises a 5′ inverted terminal repeat (ITR) sequence region and a 3′ ITR sequence region, a hSYN promoter, a human beta-globin intron region, a heavy chain signal sequence, an antibody heavy chain variable region, a heavy chain constant region, a hinge region, a furin cleavage site, a T2A linker, alight chain signal sequence, an antibody light chain variable region, alight chain constant region, and a polyadenylation signal sequence.

In some embodiments, the AAV particle genome is VGC100 and comprises a 5′ inverted terminal repeat (ITR) sequence region and a 3′ ITR sequence region, a CMVie region, a human beta-globin intron region, a heavy chain signal sequence, an antibody heavy chain variable region, a heavy chain constant region, a hinge region, a furin cleavage site, a T2A linker, alight chain signal sequence, an antibody light chain variable region, alight chain constant region, and a polyadenylation signal sequence.

TABLE 50
Viral Genome Cassettes Sequence Regions

| | VGC101 | | VGC102 | | VGC103 | | VGC104 | | VGC105 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13528 | 699 | 13528 | 699 | 13528 | 699 | 13528 | 699 | 13528 | 699 |
| Intron | 13551 | 566 | 13551 | 566 | 13551 | 566 | 13551 | 566 | 13551 | 566 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 13154 | 45 | 13157 | 75 | 13160 | 120 | 13152 | 15 | 13154 | 45 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC101 and comprises a 5′ inverted terminal repeat (ITR) sequence region and a 3′ ITR sequence region, a GFAP promoter, a human beta-globin intron region, a light chain signal sequence, an antibody light chain variable region, a (G4S)3 linker (“(G4)3” disclosed as SEQ ID NO: 13143), an antibody heavy chain variable region, an HA tag, and a polyadenylation signal sequence.

In some embodiments, the AAV particle genome is VGC102 and comprises a 5′ inverted terminal repeat (ITR) sequence region and a 3′ ITR sequence region, a GFAP promoter, a human beta-globin intron region, a light chain signal sequence, an antibody light chain variable region, a (G4S)5 linker (“(G4)5” disclosed as SEQ ID NO: 13144), an antibody heavy chain variable region, an HA tag, and a polyadenylation signal sequence.

In some embodiments, the AAV particle genome is VGC103 and comprises a 5′ inverted terminal repeat (ITR) sequence region and a 3′ ITR sequence region, a GFAP promoter, a human beta-globin intron region, a light chain signal sequence, an antibody light chain variable region, a (G4S)8 linker (“(G4)8” disclosed as SEQ ID NO: 13148), an antibody heavy chain variable region, an HA tag, and a polyadenylation signal sequence.

In some embodiments, the AAV particle genome is VGC104 and comprises a 5′ inverted terminal repeat (ITR) sequence region and a 3′ ITR sequence region, a GFAP promoter, a human beta-globin intron region, alight chain signal sequence, an antibody light chain variable region, a G4S linker (“G4” disclosed as SEQ ID NO: 13141), an antibody heavy chain variable region, an HA tag, and a polyadenylation signal sequence.

In some embodiments, the AAV particle genome is VGC105 and comprises a 5′ inverted terminal repeat (ITR) sequence region and a 3′ ITR sequence region, a GFAP promoter, a human beta-globin intron region, a light chain signal sequence, an antibody light chain variable region, a (G4S)3 linker (“(G4)3” disclosed as SEQ ID NO: 13143), an antibody heavy chain variable region, an HA tag, and a polyadenylation signal sequence.

TABLE 51
Viral Genome Cassettes Sequence Regions

| | VGC106 | | VGC107 | | VGC108 | | VGC108 | | VGC109 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13528 | 699 | 13528 | 699 | 13528 | 699 | 13529 | 557 | 13529 | 557 |
| Intron | 13551 | 566 | 13551 | 566 | 13551 | 566 | 13551 | 566 | 13551 | 566 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 1727 | 75 | 1726 | 54 | 1726 | 54 | 1727 | 75 | 1726 | 54 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC106 and comprises a 5′ inverted terminal repeat (ITR) sequence region and a 3′ ITR sequence region, a GFAP promoter, a human beta-globin intron region, a light chain signal sequence, an antibody light chain variable region, alight chain constant region, a F2A linker, a heavy chain signal sequence, an antibody heavy chain variable region, a heavy chain constant region, and a polyadenylation signal sequence.

In some embodiments, the AAV particle genome is VGC107 and comprises a 5′ inverted terminal repeat (ITR) sequence region and a 3′ ITR sequence region, a GFAP promoter, a human beta-globin intron region, a light chain signal sequence, an antibody light chain variable region, a light chain constant region, a T2A linker, a heavy chain signal sequence, an antibody heavy chain variable region, a heavy chain constant region, and a polyadenylation signal sequence.

In some embodiments, the AAV particle genome is VGC108 and comprises a 5′ inverted terminal repeat (ITR) sequence region and a 3′ ITR sequence region, a GFAP promoter, a human beta-globin intron region, a light chain signal sequence, an antibody light chain variable region, a light chain constant region, a T2A linker, a heavy chain signal sequence, an antibody heavy chain variable region, a heavy chain constant region, and a polyadenylation signal sequence.

In some embodiments, the AAV particle genome is VGC109 and comprises a 5′ inverted terminal repeat (ITR) sequence region and a 3′ ITR sequence region, a hSYN promoter, a human beta-globin intron region, alight chain signal sequence, an antibody light chain variable region, a light chain constant region, a F2A linker, a heavy chain signal sequence, an antibody heavy chain variable region, a heavy chain constant region, and a polyadenylation signal sequence.

In some embodiments, the AAV particle genome is VGC110 and comprises a 5′ inverted terminal repeat (ITR) sequence region and a 3′ ITR sequence region, a hSYN promoter, a human beta-globin intron region, alight chain signal sequence, an antibody light chain variable region, alight chain constant region, a T2A linker, a heavy chain signal sequence, an antibody heavy chain variable region, a heavy chain constant region, and a polyadenylation signal sequence.

TABLE 52
Viral Genome Cassettes Sequence Regions

| | VGC111 | | VGC112 | | VGC113 | | VGC114 | | VGC115 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13528 | 699 | 13529 | 557 | 13529 | 557 | 13529 | 557 | 13529 | 557 |
| Intron | 13551 | 566 | 13551 | 566 | 13551 | 566 | 13551 | 566 | 13551 | 566 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 13152 | 15 | 13154 | 45 | 13157 | 75 | 13160 | 120 | 13152 | 15 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC111 and comprises a 5′ inverted terminal repeat (ITR) sequence region and a 3′ ITR sequence region, a GFAP promoter, a human beta-globin intron region, a light chain signal sequence, an antibody light chain variable region, a G4S linker (“G4S” disclosed as SEQ ID NO: 13141), an antibody heavy chain variable region, an HA tag, and a polyadenylation signal sequence.

In some embodiments, the AAV particle genome is VGC112 and comprises a 5′ inverted terminal repeat (ITR) sequence region and a 3′ ITR sequence region, a hSYN promoter, a human beta-globin intron region, alight chain signal sequence, an antibody light chain variable region, a (G4S)3 linker (“(G4)3” disclosed as SEQ ID NO: 13143), an antibody heavy chain variable region, an HA tag, and a polyadenylation signal sequence.

In some embodiments, the AAV particle genome is VGC113 and comprises a 5′ inverted terminal repeat (ITR) sequence region and a 3′ ITR sequence region, a hSYN promoter, a human beta-globin intron region, alight chain signal sequence, an antibody light chain variable region, a (G4S)5 linker (“(G4)5” disclosed as SEQ ID NO: 13144), an antibody heavy chain variable region, an HA tag, and a polyadenylation signal sequence.

In some embodiments, the AAV particle genome is VGC114 and comprises a 5′ inverted terminal repeat (ITR) sequence region and a 3′ ITR sequence region, a hSYN promoter, a human beta-globin intron region, alight chain signal sequence, an antibody light chain variable region, a (G4S)8 linker (“(G4S)8” disclosed as SEQ ID NO: 13148), an antibody heavy chain variable region, an HA tag, and a polyadenylation signal sequence.

In some embodiments, the AAV particle genome is VGC115 and comprises a 5′ inverted terminal repeat (ITR) sequence region and a 3′ ITR sequence region, a hSYN promoter, a human beta-globin intron region, alight chain signal sequence, an antibody light chain variable region, a G4S linker (“G4S” disclosed as SEQ ID NO: 13141), an antibody heavy chain variable region, an HA tag, and a polyadenylation signal sequence.

TABLE 53
Viral Genome Cassettes Sequence Regions

| | VGC116 | | VGC117 | | VGC118 | | VGC119 | | VGC120 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13529 | 557 | 13529 | 557 | 13529 | 557 | 13529 | 557 | 13529 | 557 |
| Intron | 13551 | 566 | 13551 | 566 | 13551 | 566 | 13551 | 566 | 13551 | 566 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 1725 | 12 | 1724 | 12 | 1724 | 12 | 1724 | 12 | 1724 | 12 |
| Linker | 1727 | 75 | 1726 | 54 | 1726 | 54 | 1726 | 54 | 1726 | 54 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC116 and comprises a 5′ inverted terminal repeat (ITR) sequence region and a 3′ITR sequence region, a hSYN promoter, a human beta-globin intron region, a heavy chain signal sequence, an antibody heavy chain variable region, a heavy chain constant region, a furin cleavage site, a F2A linker, a light chain signal sequence, an antibody light chain variable region, alight chain constant region, and a polyadenylation signal sequence.

In some embodiments, the AAV particle genome is VGC117 and comprises a 5′ inverted terminal repeat (ITR) sequence region and a 3′ ITR sequence region, a hSYN promoter, a human beta-globin intron region, a heavy chain signal sequence, an antibody heavy chain variable region, a heavy chain constant region, a furin cleavage site, a T2A linker, a light chain signal sequence, an antibody light chain variable region, alight chain constant region, and a polyadenylation signal sequence.

In some embodiments, the AAV particle genome is VGC118 and comprises a 5′ inverted terminal repeat (ITR) sequence region and a 3′ ITR sequence region, a hSYN promoter, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC119 and comprises a 5′ inverted terminal repeat (ITR) sequence region and a 3′ ITR sequence region, a hSYN promoter, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC120 and comprises a 5′ inverted terminal repeat (ITR) sequence region and a 3 IT R sequence region, a hSYN promoter, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

TABLE 54
Viral Genome Cassettes Sequence Regions

VGC121

| Sequence Regions | Region SEQ ID NO | Region length |
|---|---|---|
| 5′ ITR | 13519 | 130 |
| Promoter | 13529 | 557 |
| Intron | 13551 | 566 |
| Signal | 13567 | 57 |
| Variable and/or Constant | — | — |
| Linker | 13151 | 198 |
| Linker | 1724 | 12 |
| Linker | 1726 | 54 |
| Signal | 13569 | 57 |
| Variable and/or Constant | — | — |
| PolyA | 13576 | 127 |
| 3′ ITR | 13521 | 130 |

In some embodiments, the AAV particle genome is VGC121 and comprises a 5′ inverted terminal repeat (ITR) sequence region and a 3′ ITR sequence region, a hSYN promoter, a human beta-globin intron region, a heavy chain signal sequence, an antibody heavy chain variable region, a heavy chain constant region, a hinge region, a furin cleavage site, a T2A linker, alight chain signal sequence, an antibody light chain variable region, alight chain constant region, and a polyadenylation signal sequence.

TABLE 55
Viral Genome Cassettes Sequence Regions

| | VGC122 | | VGC123 | | VGC124 | | VGC125 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 130 | 13519 | 130 | 13519 | 130 |
| Promoter | 13529 | 557 | 13528 | 699 | 13529 | 557 | 13534 | 380 |
| Intron | 13551 | 566 | 13551 | 566 | 13551 | 566 | 13551 | 566 |
| Signal | 13569 | 57 | 13569 | 57 | 13569 | 57 | 13569 | 57 |
| Variable | — | — | — | — | — | — | — | — |
| and/or | | | | | | | | |
| Constant | | | | | | | | |
| Linker | 1726 | 54 | 1726 | 54 | 1726 | 54 | 1726 | 54 |
| Signal | 13567 | 57 | 13567 | 57 | 13567 | 57 | 13567 | 57 |
| Variable | — | — | — | — | — | — | — | — |
| and/or | | | | | | | | |
| Constant | | | | | | | | |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC122 and comprises a 5′ inverted terminal repeat (ITR) sequence region and a 3′ ITR sequence region, a hSYN promoter, a human beta-globin intron region, alight chain signal sequence, an antibody light chain variable region, a light chain constant region, a T2A linker, a heavy chain signal sequence, an antibody heavy chain variable region, a heavy chain constant region, and a polyadenylation signal sequence.

In some embodiments, the AAV particle genome is VGC123 and comprises a 5′ inverted terminal repeat (ITR) sequence region and a 3′ ITR sequence region, a GFAP promoter, a human beta-globin intron region, a light chain signal sequence, an antibody light chain variable region, a light chain constant region, a T2A linker, a heavy chain signal sequence, an antibody heavy chain variable region, a heavy chain constant region, and a polyadenylation signal sequence.

In some embodiments, the AAV particle genome is VGC124 and comprises a 5′ inverted terminal repeat (ITR) sequence region and a 3′ITR sequence region, a hSYN promoter, a human beta-globin intron region, alight chain signal sequence, an antibody light chain variable region, alight chain constant region, a T2A linker, a heavy chain signal sequence, an antibody heavy chain variable region, a heavy chain constant region, and a polyadenylation signal sequence.

In some embodiments, the AAV particle genome is VGC125 and comprises a 5′ inverted terminal repeat (ITR) sequence region and a 3′ ITR sequence region, a CMVie region, a human beta-globin intron region, a light chain signal sequence, an antibody light chain variable region, a light chain constant region, a T2A linker, a heavy chain signal sequence, an antibody heavy chain variable region, a heavy chain constant region, and a polyadenylation signal sequence.

TABLE 56
Viral Genome Cassettes Sequence Regions

| | VGC126 | | VGC127 | |
|---|---|---|---|---|
| | Region SEQ | Region | Region SEQ | Region |
| Sequence Regions | ID NO | length | ID NO | length |
| 5′ ITR | 13519 | 130 | 13519 | 130 |
| Promoter | 13534 | 380 | 13534 | 380 |
| Promoter | 13526 | 260 | 13526 | 260 |
| Intron | 13551 | 566 | 13551 | 566 |
| Signal | 13567 | 57 | 13567 | 57 |
| Variable and/or Constant | — | — | — | — |
| Linker | 1725 | 12 | 1724 | 12 |
| Linker | 1727 | 75 | 1726 | 54 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC126 and comprises a 5′ inverted terminal repeat (ITR) sequence region and a 3′ITR sequence region, a CMVie region, a CB promoter, a human beta-globin intron region, a heavy chain signal sequence, an antibody heavy chain variable region, a heavy chain constant region, a furin cleavage site, an F2A linker, a light chain signal sequence, an antibody light chain variable region, a light chain constant region, and a polyadenylation signal sequence.

In some embodiments, the AAV particle genome is VGC127 and comprises a 5′ inverted terminal repeat (ITR) sequence region and a 3′ ITR sequence region, a CMVie region, a CB promoter, a human beta-globin intron region, a heavy chain signal sequence, an antibody heavy chain variable region, a heavy chain constant region, a furin cleavage site, an T2A linker, a light chain signal sequence, an antibody light chain variable region, alight chain constant region, and a polyadenylation signal sequence.

TABLE 57
Viral Genome Cassettes Sequence Regions

VGC128

| | Region | Region |
|---|---|---|
| Sequence Regions | SEQ ID NO | length |
| 5′ ITR | 13519 | 130 |
| Promoter | 13534 | 380 |
| Promoter | 13526 | 260 |
| Intron | 13551 | 566 |
| Signal | 13569 | 57 |
| Variable and/or Constant | — | — |
| Linker | 1727 | 75 |
| Signal | 13567 | 57 |
| Variable and/or Constant | — | — |
| PolyA | 13576 | 127 |
| 3′ ITR | 13521 | 130 |

In some embodiments, the AAV particle genome is an comprises a inverted terminal repeat (ITR) sequence region and a 3′ ITR sequence region, a CMVie region, a CB promoter, a human beta-globin intron region, alight chain signal sequence, an antibody light chain variable region, a light chain constant region, an F2A linker, a heavy chain signal sequence, an antibody heavy chain variable region, a heavy chain constant region, and a polyadenylation signal sequence.

TABLE 58
Viral Genome Cassettes Sequence Regions

| | VGC129 | | VGC130 | | VGC131 | | VGC132 | | VGC133 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| CAG promoter | 13523 | 1715 | 13523 | 1715 | 13523 | 1715 | 13523 | 1715 | 13523 | 1715 |
| CMVie | 13524 | 299 | 13524 | 299 | 13524 | 299 | 13524 | 299 | 13524 | 299 |
| CBA min. | 13525 | 283 | 13525 | 283 | 13525 | 283 | 13525 | 283 | 13525 | 283 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 1730 | 45 | 13161 | 60 | 13156 | 75 | 13158 | 90 | 13159 | 120 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC129 and comprises a 5′ inverted terminal repeat (ITR) sequence region and a 3′ITR sequence region, a CAG promoter comprising a CMVie region and a minimal CSA promoter region, a heavy chain signal sequence, an antibody heavy chain variable region, a (G4S)3 linker (“(G4S)3” disclosed as SEQ ID NO: 13143), an antibody light chain variable region, an HA tag, and a polyadenylation signal sequence.

In some embodiments, the AAV particle genome is VGC130 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a (G4S)4 linker (“(G4S)4” disclosed as SEQ ID NO: 113146), an antibody light chain variable region, an HA tag, and a polyadenylation signal sequence.

In some embodiments, the AAV particle genome is VGC131 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a (G4S)5 linker (“(G4S)5” disclosed as SEQ ID NO: 13144), an antibody light chain variable region, an HA tag, and a polyadenylation signal sequence.

In some embodiments, the AAV particle genome is VGC132 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a (G4S)6 linker (“(G4S)4” disclosed as SEQ ID NO: 13147), an antibody light chain variable region, an HA tag, and a polyadenylation signal sequence.

In some embodiments, the AAV particle genome is VGC133 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a (G4S)8 linker (“(G4)8” disclosed as SEQ ID NO: 13148), an antibody light chain variable region, an HA tag, and a polyadenylation signal sequence.

TABLE 59
Viral Genome Cassettes Sequence Regions

VGC134

| | Region | Region |
|---|---|---|
| Sequence Regions | SEQ ID NO | length |
| 5′ ITR | 13519 | 130 |
| CAG Promoter | 13523 | 1715 |
| CMVie | 13524 | 299 |
| CBA min. | 13525 | 283 |
| Signal | 13567 | 57 |
| Variable and/or Constant | — | — |
| Linker | 13152 | 15 |
| Variable and/or Constant | — | — |
| Tag | 13571 | 27 |
| PolyA | 13576 | 127 |
| 3′ ITR | 13521 | 130 |

In some embodiments, the AAV particle genome is VGC134 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a G4S linker (“G4” disclosed as SEQ ID NO: 13141), an antibody light chain variable region, an HA tag, and a polyadenylation signal sequence.

TABLE 60
Viral Genome Cassettes Sequence Regions

| | VGC135 | | VGC136 | | VGC137 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 130 | 13519 | 130 |
| CAG promoter | 13523 | 1715 | 13523 | 1715 | 13523 | 1715 |
| CMVie | 13524 | 299 | 13524 | 299 | 13524 | 299 |
| CBA min. | 13525 | 283 | 13525 | 283 | 13525 | 283 |
| Signal | 13567 | 57 | 13567 | 57 | 13567 | 57 |
| Variable | — | — | — | — | — | — |
| and/or | | | | | | |
| Constant | | | | | | |
| Linker | 1724 | 12 | 1724 | 12 | 1724 | 12 |
| Linker | 1726 | 54 | 1726 | 54 | 1726 | 54 |
| Signal | 13569 | 57 | 13569 | 57 | 13569 | 57 |
| Variable | — | — | — | — | — | — |
| and/or | | | | | | |
| Constant | | | | | | |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC135 and comprises a 5′ inverted terminal repeat (ITR) sequence region and a 3′ ITR sequence region, a CAG promoter comprising a CMVie region and a minimal CBA promoter region, a heavy chain signal sequence, an antibody heavy chain variable region, a heavy chain constant region, a furin cleavage site, a T2A linker, alight chain signal sequence, an antibody light chain variable region, a light chain constant region, and a polyadenylation signal sequence.

In some embodiments, the AAV particle genome is VGC136 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC137 and comprises a 5′ inverted terminal repeat (ITR) sequence region and a 3′ ITR sequence region, a CAG promoter comprising a CMVie region and a minimal CBA promoter region, a heavy chain signal sequence, an antibody heavy chain variable region, a heavy chain constant region, a furin cleavage site, a T2A linker, alight chain signal sequence, an antibody light chain variable region, a light chain constant region, and a polyadenylation signal sequence.

TABLE 61
Viral Genome Cassettes Sequence Regions

VGC138

| | Region | Region |
|---|---|---|
| Sequence Regions | SEQ ID NO | length |
| 5′ ITR | 13519 | 130 |
| CAG Promoter | 13523 | 1715 |
| CMVie | 13524 | 299 |
| CBA min. | 13525 | 283 |
| Signal | 13567 | 57 |
| Variable and/or Constant | — | — |
| Linker | 13151 | 198 |
| Linker | 1724 | 12 |
| Linker | 1726 | 54 |
| Signal | 13569 | 57 |
| Variable and/or Constant | — | — |
| PolyA | 13576 | 127 |
| 3′ ITR | 13521 | 130 |

In some embodiments, the AAV particle genome is VGC138 and comprises a 5′ inverted terminal repeat (ITR) sequence region and a 3′ ITR sequence region, a CAG promoter comprising a CMVie region and a minimal CBA promoter region, a heavy chain signal sequence, an antibody heavy chain variable region, a heavy chain constant region, a hinge region, a furin cleavage site, a T2A linker, alight chain signal sequence, an antibody light chain variable region, alight chain constant region, and a polyadenylation signal sequence.

TABLE 62
Viral Genome Cassettes Sequence Regions

VGC139

| | Region | Region |
|---|---|---|
| Sequence Regions | SEQ ID NO | length |
| 5′ ITR | 13519 | 130 |
| CAG Promoter | 13523 | 1715 |
| CMVie | 13524 | 299 |
| CBA min. | 13525 | 283 |
| Signal | 13567 | 57 |
| Variable and/or Constant | — | — |
| Variable and/or Constant | — | — |
| Tag | 13571 | 27 |
| PolyA | 13576 | 127 |
| 3′ ITR | 13521 | 130 |

In some embodiments, the AAV particle genome is VGC139 and comprises a 5′ inverted terminal repeat (ITR) sequence region and a 3′ITR sequence region, a CAG promoter comprising a CMVie region and a minimal CBA promoter region, a heavy chain signal sequence, an antibody heavy chain variable region, an antibody light chain variable region, an HA tag, and a polyadenylation signal sequence.

TABLE 63
Viral Genome Cassettes Sequence Regions

| | VGC140 | | VGC141 | | VGC142 | | VGC143 | | VGC144 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| CAG promoter | 13533 | 1714 | 13533 | 1714 | 13533 | 1714 | 13533 | 1714 | 13523 | 1715 |
| CMVie | 13524 | 299 | 13524 | 299 | 13524 | 299 | 13524 | 299 | 13524 | 299 |
| CBA min. | 13525 | 283 | 13525 | 283 | 13525 | 283 | 13525 | 283 | 13525 | 283 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 13154 | 45 | 13155 | 60 | 13157 | 75 | 2259 | 90 | 13152 | 15 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC140 and comprises a 5′ inverted terminal repeat (ITR) sequence region and a 3′ITR sequence region, a CAG promoter comprising a CMVie region and a minimal CBA promoter region, a light chain signal sequence, an antibody light chain variable region, a (G4S)3 linker (“(G4S)3” disclosed as SEQ ID NO: 13143), an antibody heavy chain variable region, an HA tag, and a polyadenylation signal sequence.

In some embodiments, the AAV particle genome is VGC141 and comprises a 5′ inverted terminal repeat (ITR) sequence region and a 3′ ITR sequence region, a CAG promoter comprising a CMVie region and a minimal CBA promoter region, a light chain signal sequence, an antibody light chain variable region, a (G4S)4 linker (“(G4)4” disclosed as SEQ ID NO: 13146), an antibody heavy chain variable region, an HA tag, and a polyadenylation signal sequence.

In some embodiments, the AAV particle genome is VGC142 and comprises a 5′ inverted terminal repeat (ITR) sequence region and a 3′ITR sequence region, a CAG promoter comprising a CMVie region and a minimal CBA promoter region, a light chain signal sequence, an antibody light chain variable region, a (G4S)5 linker (“(G4S)5” disclosed as SEQ ID NO: 13144), an antibody heavy chain variable region, an HA tag, and a polyadenylation signal sequence.

In some embodiments, the AAV particle genome is VGC143 and comprises a 5′ inverted terminal repeat (ITR) sequence region and a 3′ITR sequence region, a CAG promoter comprising a CMVie region and a minimal CBA promoter region, a light chain signal sequence, an antibody light chain variable region, a (G4S)6 linker (“(G4S)4” disclosed as SEQ ID NO: 13147), an antibody heavy chain variable region, an HA tag, and a polyadenylation signal sequence.

In some embodiments, the AAV particle genome is VGC144 and comprises a 5′ inverted terminal repeat (ITR) sequence region and a 3′ITR sequence region, a CAG promoter comprising a CMVie region and a minimal CBA promoter region, a light chain signal sequence, an antibody light chain variable region, a G4S linker (“G4S” disclosed a SEQ ID NO: 13141), an antibody heavy chain variable region, an HA tag, and a polyadenylation signal sequence.

TABLE 64
Viral Genome Cassettes Sequence Regions

| | VGC145 | | VGC146 | | VGC147 | | VGC148 | | VGC149 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13528 | 699 | 13528 | 699 | 13528 | 699 | 13528 | 699 | 13529 | 557 |
| Intron | 13551 | 566 | 13551 | 566 | 13551 | 566 | 13551 | 566 | 13551 | 566 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 13152 | 15 | 1730 | 45 | 13156 | 75 | 13159 | 120 | 1730 | 45 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC145 and comprises a 5′ inverted terminal repeat (ITR) sequence region and a 3′ ITR sequence region, a GFAP promoter, a human beta-globin intron region, a heavy chain signal sequence, an antibody heavy chain variable region, a G4S linker (“G4S” disclosed as SEQ ID NO: 13141), an antibody light chain variable region, an HA tag, and a polyadenylation signal sequence.

In some embodiments, the AAV particle genome is VGC146 and comprises a 5′ inverted terminal repeat (ITR) sequence region and a 3′ ITR sequence region, a GFAP promoter, a human beta-globin intron region, a heavy chain signal sequence, an antibody heavy chain variable region, a (G4S)3 linker (“(G4S)3” disclosed as SEQ ID NO: 13143), an antibody light chain variable region, an HA tag, and a polyadenylation signal sequence.

In some embodiments, the AAV particle genome is VGC147 and comprises a 5′ inverted terminal repeat (ITR) sequence region and a 3′ ITR sequence region, a GFAP promoter, a human beta-globin intron region, a heavy chain signal sequence, an antibody heavy chain variable region, a (G4S)5 linker (“(G4S)5” disclosed as SEQ ID NO: 13144), an antibody light chain variable region, an HA tag, and a polyadenylation signal sequence.

In some embodiments, the particle genome is VGC148 and comprises a 5′ inverted terminal repeat (ITR) sequence region and a 3′ITR sequence region, a GFAP promoter, a human beta-globin intron region, a heavy chain signal sequence, an antibody heavy chain variable region, a (G4S)8 linker (“(G4S)8” disclosed as SEQ ID NO: 13148), an antibody light chain variable region, an HA tag, and a polyadenylation signal sequence.

In some embodiments, the AAV particle genome is VGC149 and comprises a 5′ inverted terminal repeat (ITR) sequence region and a 3′ITR sequence region, a hSYN promoter, a human beta-globin intron region, a heavy chain signal sequence, an antibody heavy chain variable region, a (G4S)3 linker (“(G4S)3” disclosed as SEQ ID NO: 13143), an antibody light chain variable region, an HA tag and a polyadenylation signal sequence.

TABLE 65
Viral Genome Cassettes Sequence Regions

| | VGC150 | | VGC151 | | VGC152 | | VGC153 | | VGC154 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13528 | 699 | 13529 | 557 | 13529 | 557 | 13529 | 557 | 13534 | 380 |
| Intron | 13551 | 566 | 13551 | 566 | 13551 | 566 | 13551 | 566 | 13551 | 566 |
| Signal | 13569 | 57 | 13569 | 57 | 13569 | 57 | 13569 | 57 | 13569 | 57 |
| Variable | — | — | — | — | — | — | — | — | — | — |
| and/or | | | | | | | | | | |
| Constant | | | | | | | | | | |
| Linker | 13157 | 75 | 13154 | 45 | 13157 | 75 | 13152 | 15 | 13152 | 15 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC150 and comprises a 5′ inverted terminal repeat (ITR) sequence region and a 3° ITR sequence region, a GFAP promoter, a human beta-globin intron region, a light chain signal sequence, an antibody light chain variable region, a (G4S)5 linker (“(G4S)5” disclosed as SEQ ID NO: 13144), an antibody heavy chain variable region, an HA tag, and a polyadenylation signal sequence.

In some embodiments, the AAV particle genome is VGC151 and comprises a 5′ inverted terminal repeat (ITR) sequence region and a 3′ ITR sequence region, a hSYN promoter, a human beta-globin intron region, a light chain signal sequence, an antibody light chain variable region, a (G4S)3 linker (“(G4S)3” disclosed as SEQ ID NO: 13143), an antibody heavy chain variable region, an HA tag, and a polyadenylation signal sequence.

In some embodiments, the AAV particle genome is VGC152 and comprises a 5′ inverted terminal repeat (ITR) sequence region and a 3′ ITR sequence region, a hSYN promoter, a human beta-globin intron region, a light chain signal sequence, an antibody light chain variable region, a (G4S)5 linker (“(G4S)5” disclosed as SEQ ID NO: 13144), an antibody heavy chain variable region, an HA tag, and a polyadenylation signal sequence.

In some embodiments, the AAV particle genome is VGC153 and comprises a 5′ inverted terminal repeat (ITR) sequence region and a 3′ ITR sequence region, a hSYN promoter, a human beta-globin intron region, alight chain signal sequence, an antibody light chain variable region, a G4S linker (“G4” disclosed as SEQ ID NO: 13141), an antibody heavy chain variable region, an HA tag, and a polyadenylation signal sequence.

In some embodiments, the AAV particle genome is VGC154 and comprises a 5′ inverted terminal repeat (ITR) sequence region and a 3′ ITR sequence region, a CMVie region, a human beta-globin intron region, alight chain signal sequence, an antibody light chain variable region, a G4S linker (“G4” disclosed as SEQ ID NO: 13141), an antibody heavy chain variable region, an HA tag, and a polyadenylation signal sequence.

TABLE 66
Viral Genome Cassettes Sequence Regions

| | VGC155 | | VGC156 | | VGC157 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 130 | 13519 | 130 |
| Promoter | 13529 | 557 | 13529 | 557 | 13529 | 557 |
| Intron | 13551 | 566 | 13551 | 566 | 13551 | 566 |
| Signal | 13567 | 57 | 13567 | 57 | 13567 | 57 |
| Variable | — | — | — | — | — | — |
| and/or | | | | | | |
| Constant | | | | | | |
| Linker | 13156 | 75 | 13159 | 120 | 13152 | 15 |
| Variable | — | — | — | — | — | — |
| and/or | | | | | | |
| Constant | | | | | | |
| Tag | 13571 | 27 | 13571 | 27 | 13571 | 27 |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC155 and comprises a 5′ inverted terminal repeat (ITR) sequence region and a 3′ ITR sequence region, a hSYN promoter, a human beta-globin intron region, a heavy chain signal sequence, an antibody heavy chain variable region, a (G4S)5 linker (“(G4S)5” disclosed as SEQ ID NO: 13144), an antibody light chain variable region, an HA tag, and a polyadenylation signal sequence.

In some embodiments, the AAV particle genome is VGC156 and comprises a 5′ inverted terminal repeat (ITR) sequence region and a 3′ ITR sequence region, a hSYN promoter, a human beta-globin intron region, a heavy chain signal sequence, an antibody heavy chain variable region, a (G4S)8 linker (“(G4)8” disclosed as SEQ ID NO: 13148), an antibody light chain variable region, an HA tag, and a polyadenylation signal sequence.

In some embodiments, the AAV particle genome is VGC157 and comprises a 5′ inverted terminal repeat (ITR) sequence region and a 3′ ITR sequence region, a hSYN promoter, a human beta-globin intron region, a heavy chain signal sequence, an antibody heavy chain variable region, a G4S linker (“4” disclosed as SEQ ID NO: 13141), an antibody light chain variable region, an HA tag, and a polyadenylation signal sequence.

TABLE 67
Viral Genome Cassettes Sequence Regions

| | VGC158 | | VGC159 | | VGC160 | | VGC161 | | VGC162 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| Promoter | 13534 | 380 | 13534 | 380 | 13534 | 380 | 13534 | 380 | 13534 | 380 |
| Intron | 13551 | 566 | 13551 | 566 | 13551 | 566 | 13551 | 566 | 13551 | 566 |
| Signal | 13567 | 57 | 13567 | 57 | 13567 | 57 | 13567 | 57 | 13567 | 57 |
| Variable | — | — | — | — | — | — | — | — | — | — |
| and/or | | | | | | | | | | |
| Constant | | | | | | | | | | |
| Linker | 13152 | 15 | 13153 | 30 | 1730 | 45 | 13161 | 60 | 13156 | 75 |
| Variable | — | — | — | — | — | — | — | — | — | — |
| and/or | | | | | | | | | | |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC158 and comprises a 5′ inverted terminal repeat (ITR) sequence region and a 3′ ITR sequence region, a CMVie region, a human beta-globin intron region, a heavy chain signal sequence, an antibody heavy chain variable region, alight chain constant region, a G4S linker (“G4S” disclosed as SEQ ID NO: 13141), an antibody light chain variable region, an HA tag, and a polyadenylation signal sequence.

In some embodiments, the AAV particle genome is VGC159 and comprises a 5′ inverted terminal repeat (ITR) sequence region and a 3′ITR sequence region, a CMVie region, a human beta-globin intron region, a heavy chain signal sequence, an antibody heavy chain variable region, alight chain constant region, a (G4S)2 linker (“(G4S)4” disclosed as SEQ ID NO: 13145), an antibody light chain variable region, an HA tag, and a polyadenylation signal sequence.

In some embodiments, the AAV particle genome is VGC160 and comprises a 5′ inverted terminal repeat (ITR) sequence region and a 3′ ITR sequence region, a CMVie region, a human beta-globin intron region, a heavy chain signal sequence, an antibody heavy chain variable region, alight chain constant region, a (G4S)3 linker (“(G4S)3” disclosed as SEQ ID NO: 13143), an antibody light chain variable region, an HA tag, and a polyadenylation signal sequence.

In some embodiments, the AAV particle genome is VGC161 and comprises a 5′ inverted terminal repeat (ITR) sequence region and a 3′ ITR sequence region, a CMVie region, a human beta-globin intron region, a heavy chain signal sequence, an antibody heavy chain variable region, alight chain constant region, a (G4S)4 linker (“(G4S)4” disclosed as SEQ ID NO: 13146), an antibody light chain variable region, an HA tag, and a polyadenylation signal sequence.

In some embodiments, the AAV particle genome is VGC162 and comprises a 5′ inverted terminal repeat (ITR) sequence region and a 3′ ITR sequence region, a CMVie region, a human beta-globin intron region, a heavy chain signal sequence, an antibody heavy chain variable region, alight chain constant region, a (G4S)5 linker (“(G4)5” disclosed as SEQ ID NO: 13144), an antibody light chain variable region, an HA tag, and a polyadenylation signal sequence.

TABLE 68
Viral Genome Cassettes Sequence Regions

| | VGC163 | | VGC164 | | VGC165 | | VGC166 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 130 | 13519 | 130 | 13519 | 130 |
| Promoter | 13534 | 380 | 13534 | 380 | 13534 | 380 | 13534 | 380 |
| Intron | 13551 | 566 | 13551 | 566 | 13551 | 566 | 13551 | 566 |
| Signal | 13567 | 57 | 13570 | 57 | 13570 | 57 | 13570 | 57 |
| Variable and/or | — | — | — | — | — | — | — | — |
| Constant | | | | | | | | |
| Linker | 13158 | 90 | 1724 | 12 | 1724 | 12 | 1724 | 12 |
| Linker | — | — | 1726 | 54 | 1726 | 54 | 1726 | 54 |
| Signal | — | — | — | — | 13569 | 57 | 13569 | 57 |
| Variable and/or | — | — | — | — | — | — | — | — |
| Constant | | | | | | | | |
| Tag | 13571 | 27 | — | — | — | — | — | — |
| PolyA | 13576 | 127 | 13576 | 127 | 13576 | 127 | 13576 | 127 |
| 3′ ITR | 13521 | 130 | 13521 | 130 | 13521 | 136 | 13521 | 130 |

In some embodiments, the AAV particle genome is VGC163 an comprises a 5′ inverted terminal repeat (ITR) sequence region and a 3′ ITR sequence region, a CMVie region, a human beta-globin intron region, a heavy chain signal sequence, an antibody heavy chain variable region, a light chain constant region, a (G4S)6 linker (“(G4S)4” disclosed as SEQ ID NO: 13147), an antibody light chain variable region, an HA tag, and a polyadenylation signal sequence.

In some embodiments, the AAV particle genome is VGC164 and comprises a 5′ inverted terminal repeat (ITR) sequence region and a 3′ ITR sequence region, a CMVie region, a human beta-globin intron region, a heavy chain signal sequence, an antibody heavy chain variable region, a heavy chain constant region, a furin cleavage site, a T2A linker, a light chain constant region, and a polyadenylation signal sequence.

In some embodiments, the AAV particle genome is VGC165 and comprises a 5′ inverted terminal repeat (ITR) sequence region and a 3′ITR sequence region, a CMVie region, a human beta-globin intron region, a heavy chain signal sequence, an antibody heavy chain variable region, a heavy chain constant region, a furin cleavage site, a T2A linker, alight chain signal sequence, an antibody light chain variable region, alight chain constant region, and a polyadenylation signal sequence.

In some embodiments, the AAV particle genome is VGC166 and comprises a 5′ inverted terminal repeat (ITR) sequence region and a 3′ ITR sequence region, a CMVie region, a human beta-globin intron region, a heavy chain signal sequence, an antibody heavy chain variable region, a heavy chain constant region, a furin cleavage site, a T2A linker, a light chain signal sequence, an antibody light chain variable region, a light chain constant region, and a polyadenylation signal sequence.

TABLE 69
Viral Genome Cassettes Sequence Regions

| | VGC167 | | VGC168 | | VGC169 | | VGC176 | | VGC171 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 136 | 13519 | 130 | 13519 | 130 | 13519 | 130 | 13519 | 130 |
| Promoter | 13534 | 386 | 13534 | 380 | 13534 | 380 | 13534 | 380 | 13534 | 380 |
| Intron | 13551 | 566 | 13551 | 566 | 13551 | 566 | 13551 | 566 | 13551 | 566 |
| Signal | 13569 | 57 | 13569 | 57 | 13569 | 57 | 13569 | 57 | 13569 | 57 |
| Variable and/or | — | — | — | — | — | — | — | — | — | — |
| Constant | | | | | | | | | | |
| Linker | 13153 | 30 | 1730 | 45 | 13161 | 60 | 13156 | 75 | 13158 | 90 |
| Variable and/or | — | — | — | — | — | — | — | — | — | — |
| Constant | | | | | | | | | | |
| Tag | 13571 | 27 | 13571 | 27 | 13571 | 27 | 13571 | 27 | 13571 | 27 |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC167 and comprises a 5′ inverted terminal repeat (ITR) sequence region and a 3′ ITR sequence region, a CMVie region, a human beta-globin intron region, a light chain signal sequence, an antibody light chain variable region, a (G4S)2 linker (“(G4)4” disclosed as SEQ ID NO: 13145), an antibody heavy chain variable region, an HA tag, and a polyadenylation signal sequence.

In some embodiments, the AAV particle genome is VGC168 and comprises a 5′ inverted terminal repeat (ITR) sequence region and a 3′ ITR sequence region, a CMVie region, a human beta-globin intron region, alight chain signal sequence, an antibody light chain variable region, a (G4S)3 linker (“(G4S)3” disclosed as SEQ ID NO: 13143), an antibody heavy chain variable region, an HA tag, and a polyadenylation signal sequence.

In some embodiments, the AAV particle genome is VGC169 and comprises a 5′ inverted terminal repeat (ITR) sequence region and a 3′ ITR sequence region, a CMVie region, a human beta-globin intron region, a light chain signal sequence, an antibody light chain variable region, a (G4S)4 linker (“(G48)4” disclosed as SEQ ID NO: 13146), an antibody heavy chain variable region, an HA tag, and a polyadenylation signal sequence.

In some embodiments, the AAV particle genome is VGC170 and comprises a 5′ inverted terminal repeat (ITR) sequence region and a 3′ ITR sequence region, a CMVie region, a human beta-globin intron region, alight chain signal sequence, an antibody light chain variable region, a (G4S)5 linker (“(G4S)5” disclosed as SEQ ID NO: 13144), an antibody heavy chain variable region, an HA tag, and a polyadenylation signal sequence.

In some embodiments, the AAV particle genome is VGC171 and comprises a 5′ inverted terminal repeat (ITR) sequence region and a 3′ ITR sequence region, a CMVie region, a human beta-globin intron region, a light chain signal sequence, an antibody light chain variable region, a (G4S)6 linker (“(G4S)4” disclosed as SEQ ID NO: 13147), an antibody heavy chain variable region, an HA tag, and a polyadenylation signal sequence.

TABLE 70
Viral Genome Cassettes Sequence Regions

| | VGC172 | | VGC173 | |
|---|---|---|---|---|
| | Region | Region | Region | Region |
| Sequence Regions | SEQ ID NO | length | SEQ ID NO | length |
| 5′ ITR | 13519 | 130 | 13520 | 141 |
| CAG Promoter | 13523 | 1715 | 13523 | 1715 |
| CMVie | 13524 | 299 | 13524 | 299 |
| CBA min. | 13525 | 283 | 13525 | 283 |
| Variable and/or Constant | — | — | — | — |
| Linker | 1724 | 12 | 1730 | 45 |
| Linker | 1726 | 54 | — | — |
| Variable and/or Constant | — | — | — | — |
| PolyA | 13576 | 127 | 13577 | 477 |
| Filler | — | — | 13580 | 1240 |
| 3′ ITR | 13521 | 130 | 13522 | 141 |

In some embodiments, the AAV particle genome is VGC172 and comprises a 5′ inverted terminal repeat (ITR) sequence region and a 3′ ITR sequence region, a CAG promoter comprising a CMVie region and a minimal CBA promoter region, an antibody heavy chain variable region, a furin cleavage site, a T2A linker, an antibody light chain variable region and a polyadenylation signal sequence.

In some embodiments, the AAV particle genome is VGC173 and comprises a 5′ inverted terminal repeat (ITR) sequence region and a 3′ ITR sequence region, a CAG promoter comprising a CMVie region and a minimal CBA promoter region, an antibody heavy chain variable region, a furin cleavage site, an antibody light chain variable region, a polyadenylation signal sequence and a filler sequence.

TABLE 71
Viral Genome Cassettes Sequence Regions

| | VGC174 | | VGC175 | | VGC176 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 130 | 13519 | 130 |
| Promoter | 13527 | 654 | 13528 | 699 | 5086 | 557 |
| Intron | 13551 | 566 | 13551 | 566 | 13551 | 566 |
| Variable and/or | — | — | — | — | — | — |
| Constant | | | | | | |
| Linker | 1724 | 12 | 1724 | 12 | 1724 | 12 |
| Linker | 1726 | 54 | 1726 | 54 | 1726 | 54 |
| Variable and/or | — | — | — | — | — | — |
| Constant | | | | | | |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC174 and comprises a 5′ inverted terminal repeat (ITR) sequence region and a 3′ITR sequence region, a CBA promoter, a human beta-globin intron region, an antibody heavy chain variable region, a furin cleavage site, a T2A linker, an antibody light chain variable region, and a polyadenylation sequence.

In some embodiments, the AAV particle genome is VGC175 and comprises a 5′ inverted terminal repeat (ITR) sequence region and a 3′ITR sequence region, a GFAP promoter, a human beta-globin intron region, an antibody heavy chain variable region, a furin cleavage site, a T2A linker, an antibody light chain variable region, and a polyadenylation sequence.

In some embodiments, the AAV particle genome is VGC176 and comprises a 5′ inverted terminal repeat (ITR) sequence region and a 3′ITR sequence region, a synapsin promoter, a human beta-globin intron region, an antibody heavy chain variable region, a furin cleavage site, a T2A linker, an antibody light chain variable region, and a polyadenylation sequence.

TABLE 72
Viral Genome Cassettes Sequence Regions

| | VGC177 | | VGC178 | | VGC179 | | VGC180 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13520 | 141 | 13520 | 141 | 13520 | 141 | 13520 | 141 |
| CBA Promoter | 13527 | 654 | 13527 | 654 | 13527 | 654 | 13527 | 654 |
| Intron | 13552 | 491 | 13553 | 387 | 13554 | 292 | — | — |
| Variable and/or | — | — | — | — | — | — | — | — |
| Constant | | | | | | | | |
| Linker | 1730 | 45 | 1730 | 45 | 1730 | 45 | 1730 | 45 |
| Variable and/or | — | — | — | — | — | — | — | — |
| Constant | | | | | | | | |
| HA Tag | 13571 | 27 | 13571 | 27 | 13571 | 27 | — | — |
| PolyA | 13576 | 127 | 13576 | 127 | 13576 | 127 | 13577 | 477 |
| Filler | — | — | — | — | — | — | 13580 | 1240 |
| 3′ ITR | 13522 | 141 | 13522 | 141 | 13522 | 141 | 13522 | 141 |

In some embodiments, the AAV particle genome is VGC177 and comprises a 5′ inverted terminal repeat (ITR) sequence region and a 3′ITR sequence region, a CAB promoter, a human beta-globin intron region, an antibody heavy chain variable region, a furin cleavage site, an antibody light chain variable region, a HA tag and a polyadenylation sequence.

In some embodiments, the AAV particle genome is VGC173 and comprises a 5′ inverted terminal repeat (ITR) sequence region and a 3′ITR sequence region, a CBA promoter, a human beta-globin intron region, an antibody heavy chain variable region, a furin cleavage site, an antibody light chain variable region, a HA tag and a polyadenylation sequence.

In some embodiments, the AAV particle genome is VGC179 and comprises a 5′ inverted terminal repeat (ITR) sequence region and a 3′ITR sequence region, a CBA promoter, a human beta-globin intron region, an antibody heavy chain variable region, a furin cleavage site, an antibody light chain variable region, a HA tag and a polyadenylation sequence.

In some embodiments, the AAV particle genome is VGC180 and comprises a 5′ inverted terminal repeat (ITR) sequence region and a 3′ITR sequence region, a CBA promoter, an antibody heavy chain variable region, a furin cleavage site, an antibody light chain variable region, a polyadenylation signal sequence and a filler sequence.

TABLE 73
Viral Genome Cassettes Sequence Regions

| | VGC181 | | VGC181 | | VGC182 | | VGC182 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 130 | 13519 | 130 | 13519 | 130 |
| CAG promoter | 13523 | 1715 | 13523 | 1715 | 13523 | 1715 | 13523 | 1715 |
| CMVie | 13524 | 299 | 13524 | 299 | 13524 | 299 | 13524 | 299 |
| CBA min. | 13525 | 283 | 13525 | 283 | 13525 | 283 | 13525 | 283 |
| Signal | 13567 | 57 | 13567 | 57 | 13567 | 57 | 13567 | 57 |
| Variable and/or | — | — | — | — | — | — | — | — |
| Constant | | | | | | | | |
| Linker | 13154 | 45 | 13154 | 45 | 13154 | 45 | 13154 | 45 |
| Variable and/or | — | — | — | — | — | — | — | — |
| Constant | | | | | | | | |
| HA Tag | 13571 | 27 | 13571 | 27 | 13571 | 27 | 13571 | 27 |
| PolyA | 13576 | 127 | 13576 | 127 | 13576 | 127 | 13576 | 127 |
| 3′ ITR | 13521 | 130 | 13521 | 130 | 13521 | 136 | 13521 | 130 |

In some embodiments, the AAV particle genome is VGC181 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable region, a (G4S)3 linker, a light chain signal, an antibody light chain variable region, a HA tag, and a polyadenylation signal sequence.

In some embodiments, the AAV particle genome is VGC182 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable region, a (G4S)3 linker, alight chain signal, an antibody light chain variable region, a HA tag, and a polyadenylation signal sequence.

In some embodiments, the AAV particle genome is VGC183 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable region, a (G4S)3 linker, alight chain signal, an antibody light chain variable region, a HA tag, and a polyadenylation signal sequence.

In some embodiments, the AAV particle genome is VGC184 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable region, a (G4S)3 linker, a light chain signal, an antibody light chain variable region, a HA tag, and a polyadenylation signal sequence

TABLE 74
Viral Genome Cassettes Sequence Regions

| | VGC185 | | VGC186 | | VGC187 | | VGC188 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 136 | 13519 | 136 | 13519 | 136 |
| SAG promoter | 13523 | 1715 | 13523 | 1715 | 13523 | 1715 | 13523 | 1715 |
| CMVie | 13524 | 299 | 13524 | 299 | 13524 | 299 | 13524 | 299 |
| CBA min. | 13525 | 283 | 13525 | 283 | 13525 | 283 | 13525 | 283 |
| Signal | 13567 | 57 | 13567 | 57 | 13567 | 57 | 13567 | 57 |
| Variable and/or | — | — | — | — | — | — | — | — |
| Constant | | | | | | | | |
| Linker | 13154 | 45 | 13154 | 45 | 13154 | 45 | 13154 | 45 |
| Variable and/or | — | — | — | — | — | — | — | — |
| Constant | | | | | | | | |
| HA Tag | 13571 | 27 | 13571 | 27 | 13571 | 27 | 13571 | 27 |
| PolyA | 13576 | 127 | 13576 | 127 | 13576 | 127 | 13576 | 127 |
| 3′ ITR | 13521 | 130 | 13521 | 136 | 13521 | 136 | 13521 | 130 |

In some embodiments, the AAV particle genome is VGC185 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable region, a (G4S)3 linker, a light chain signal, an antibody light chain variable region, a HA tag, and a polyadenylation signal sequence.

In some embodiments, the AAV particle genome is VGC186 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable region, a (G4S)3 linker, alight chain signal, an antibody light chain variable region, a HA tag, and a polyadenylation signal sequence.

In some embodiments, the AAV particle genome is VGC187 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable region, a (G4S)3 linker, alight chain signal, an antibody light chain variable region, a HA tag, and a polyadenylation signal sequence.

In some embodiments, the AAV particle genome is VGC188 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable region, a (G4S)3 linker, a light chain signal, an antibody light chain variable region, a HA tag, and a polyadenylation signal sequence.

TABLE 75
ViraI Genome Cassettes Sequence Regions

| | VGC189 | | VGC190 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| CAG Promoter | 13523 | 1715 | 13523 | 1715 |
| CMVie | 13524 | 299 | 13524 | 299 |
| CBA min. | 13525 | 283 | 13525 | 283 |
| Signal | 13567 | 57 | 13567 | 57 |
| Variable and/or Constant | — | — | — | — |
| Linker | 13153 | 30 | 13155 | 60 |
| Variable and/or Constant | — | — | — | — |
| HA Tag | 13571 | 27 | 13571 | 27 |
| PolyA | 13576 | 127 | 13576 | 127 |
| 3′ ITR | 13521 | 130 | 13521 | 130 |

In some embodiments, the AAV particle genome is VGC189 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable region, a (G4S)2 linker, a light chain signal, an antibody light chain variable region, a HA tag, and a polyadenylation signal sequence.

In some embodiments, the AAV particle genome is VGC190 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable region, a (G4S)4 linker, alight chain signal, an antibody light chain variable region, a HA tag, and a polyadenylation signal sequence.

TABLE 76
Viral Genome Cassettes Sequence Regions

| | VGC191 | | VGC192 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| CAG Promoter | 13523 | 1715 | 13523 | 1715 |
| CMVie | 13524 | 299 | 13524 | 299 |
| CBA min. | 13525 | 283 | 13525 | 283 |
| Signal | 13567 | 57 | 13567 | 57 |
| Variable and/or Constant | — | — | — | — |
| Linker | 1725 | 12 | 1725 | 12 |
| Linker | 1727 | 75 | 1727 | 75 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC191 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable and constant region, a furin cleavage site, a F2A linker, alight chain signal, an antibody light chain variable and constant region and a polyadenylation signal sequence.

In some embodiments, the AAV particle genome is VGC192 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable and constant region, a furin cleavage site, a F2A linker, a light chain signal, an antibody light chain variable and constant region and a polyadenylation signal sequence.

TABLE 77
Viral Genome Cassettes Sequence Regions

| | VGC193 | | VGC194 | | VGC195 | | VGC196 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 130 | 13519 | 130 | 13519 | 130 |
| CAG promoter | 13523 | 1715 | 13523 | 1715 | 13523 | 1715 | 13523 | 1715 |
| CMVie | 13524 | 299 | 13524 | 299 | 13524 | 299 | 13524 | 299 |
| CBA min. | 13525 | 283 | 13525 | 283 | 13525 | 283 | 13525 | 283 |
| Signal | 13567 | 57 | 13567 | 57 | 13567 | 57 | 13567 | 57 |
| Variable and/or | — | — | — | — | — | — | — | — |
| Constant | | | | | | | | |
| Linker | 1724 | 12 | 1724 | 12 | 1724 | 12 | 1724 | 12 |
| Linker | 1726 | 54 | 1726 | 54 | 1726 | 54 | 1726 | 54 |
| Signal | 13569 | 57 | 13569 | 57 | 13569 | 57 | 13569 | 57 |
| Variable and/or | — | — | — | — | — | — | — | — |
| Constant | | | | | | | | |
| PolyA | 1357S | 127 | 13576 | 127 | 13576 | 127 | 13576 | 127 |
| 3′ ITR | 13521 | 136 | 13521 | 136 | 13521 | 130 | 13521 | 136 |

In some embodiments, the AAV particle genome is VGC193 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable and constant region, a furin cleavage site, a T2A linker, a light chain signal, an antibody light chain variable and constant region and a polyadenylation signal sequence.

In some embodiments, the AAV particle genome is VGC194 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable and constant region, a furin cleavage site, a T2A linker, a light chain signal, an antibody light chain variable and constant region and a polyadenylation signal sequence.

In some embodiments, the AAV particle genome is VGC195 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable and constant region, a furin cleavage site, a T2A linker, alight chain signal, an antibody light chain variable and constant region and a polyadenylation signal sequence.

In some embodiments, the AAV particle genome is VGC196 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable and constant region, a furin cleavage site, a T2A linker, alight chain signal, an antibody light chain variable and constant region and a polyadenylation signal sequence.

TABLE 78
Viral Genome Cassettes Sequence Regions

| | VGC197 | | VGC198 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| CAG Promoter | 13523 | 1715 | 13523 | 1715 |
| CMVie | 13524 | 299 | 13524 | 299 |
| CBA min. | 13525 | 283 | 13525 | 283 |
| Signal | 13567 | 57 | 13567 | 57 |
| Variable and/or Constant | — | — | — | — |
| Linker | 1737 | 198 | 1737 | 198 |
| Linker | 1724 | 12 | 1724 | 12 |
| Linker | 1727 | 75 | 1726 | 54 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC197 and comprises a 5′ inverted terminal repeat (ITR) sequence region and a 3′ITR sequence region, a CAG promoter comprising a CMVie region and a minimal CBA promoter region, a heavy chain signal, an antibody heavy chain variable and constant region, a hinge region, a furin cleavage site, a F2A linker, a light chain signal, an antibody light chain variable and constant region and a polyadenylation signal sequence.

In some embodiments, the AAV particle genome is VGC198 and comprises a 5′ inverted terminal repeat (ITR) sequence region and a 3′ ITR sequence region, a CAG promoter comprising a CMVie region and a minimal CBA promoter region, a heavy chain signal, an antibody heavy chain variable and constant region, a hinge region, a furin cleavage site, a T2A linker, a light chain signal, an antibody light chain variable and constant region and a polyadenylation signal sequence.

TABLE 79
Viral Genome Cassettes Sequence Regions

| | VGC199 | | VGC200 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| CAG Promoter | 13523 | 1715 | 13523 | 1715 |
| CMVie | 13524 | 299 | 13524 | 299 |
| CBA min. | 13525 | 283 | 13525 | 283 |
| Signal | 13569 | 57 | 13567 | 57 |
| Variable and/or Constant | — | — | — | — |
| Linker | 13154 | 45 | 13154 | 45 |
| Variable and/or Constant | — | — | — | — |
| HA Tag | 13571 | 27 | 13571 | 27 |
| PolyA | 13576 | 127 | 13576 | 127 |
| 3′ ITR | 13521 | 130 | 13521 | 130 |

In some embodiments, the AAV particle genome is VGC199 and comprises a 5′ inverted terminal repeat (ITR) sequence region and a 3′ ITR sequence region, a CAG promoter comprising a CMVie region and a minimal CBA promoter region, a light chain signal, an antibody heavy chain variable region, a (G4S)3 linker, an antibody light chain variable region, a HA tag and a polyadenylation signal sequence.

In some embodiments, the AAV particle genome is VGC200 and comprises a 5′ inverted terminal repeat (ITR) sequence region and a 3′ ITR sequence region, a CAG promoter comprising a CMVie region and a minimal CBA promoter region, a heavy chain signal, an antibody light chain variable region, a (G4S)3 linker, an antibody heavy chain variable region, a HA tag and a polyadenylation signal sequence.

TABLE 80
Viral Genome Cassettes Sequence Regions

| | VGC201 | | VGC202 | | VGC203 | | VGC204 | |
|---|---|---|---|---|---|---|---|---|
| | Region | | Region | | Region | | Region | |
| Sequence | SEQ ID | Region | SEQ ID | Region | SEQ ID | Region | SEQ ID | Region |
| Regions | NO | length | NO | length | NO | length | NO | length |
| 5′ ITR | 13519 | 130 | 13519 | 139 | 13519 | 130 | 13519 | 130 |
| CAG promoter | 13523 | 1715 | 13523 | 1715 | 13523 | 1715 | 13523 | 1715 |
| CMVie | 13524 | 299 | 13524 | 299 | 13524 | 299 | 13524 | 299 |
| CBA min. | 13525 | 283 | 13525 | 283 | 13525 | 283 | 13525 | 283 |
| Signal | 13569 | 57 | 13569 | 57 | 13569 | 57 | 13569 | 57 |
| Variable and/or | — | — | — | — | — | — | — | — |
| Constant | | | | | | | | |
| Linker | 13153 | 39 | 13154 | 45 | 13154 | 45 | 13155 | 60 |
| Variable and/or | — | — | — | — | — | — | — | — |
| Constant | | | | | | | | |
| HA Tag | 13571 | 27 | 13571 | 27 | 13575 | 18 | 13571 | 27 |
| PolyA | 13576 | 127 | 13576 | 127 | 13576 | 127 | 13576 | 127 |
| 3′ ITR | 13521 | 130 | 13521 | 130 | 13521 | 130 | 13521 | 130 |

In some embodiments, the AAV particle genome is VGC201 and comprises a 5′ inverted terminal repeat (ITR) sequence region and a 3′ ITR sequence region, a CAG promoter comprising a CMVie region and a minimal CBA promoter region, a light chain signal, an antibody light chain variable region, a (G4S)2 linker, an antibody heavy chain variable region, a HA tag and a polyadenylation signal sequence.

In some embodiments, the AAV particle genome is VGC202 and comprises a 5′ inverted terminal repeat (ITR) sequence region and a 3′ ITR sequence region, a CAG promoter comprising a CMVie region and a minimal CBA promoter region, a light chain signal, an antibody light chain variable region, a (G4S)3 linker, an antibody heavy chain variable region, a HA tag and a polyadenylation signal sequence.

In some embodiments, the AAV particle genome is VGC203 and comprises a 5′ inverted terminal repeat (ITR) sequence region and a 3′ ITR sequence region, a CAG promoter comprising a CMVie region and a minimal CBA promoter region, a light chain signal, an antibody light chain variable region, a (G4S)3 linker, an antibody heavy chain variable region, a HA tag and a polyadenylation signal sequence.

In some embodiments, the AAV particle genome is VGC204 and comprises a 5′ inverted terminal repeat (ITR) sequence region and a 3′ ITR sequence region, a CAG promoter comprising a CMVie region and a minimal CBA promoter region, a light chain signal, an antibody light chain variable region, a (G4S)4 linker, an antibody heavy chain variable region, a HA tag and a polyadenylation signal sequence.

TABLE 81
Viral Genome Cassettes Sequence Regions

| | VGC205 | | VGC206 | | VGC207 | | VGC208 | | VGC209 | |
|---|---|---|---|---|---|---|---|---|---|---|
| | Region | | Region | | Region | | Region | | Region | |
| Sequence | SEQ ID | Region | SEQ ID | Region | SEQ ID | Region | SEQ ID | Region | SEQ ID | Region |
| Regions | NO | length | NO | length | NO | length | NO | length | NO | length |
| 5′ ITR | 13519 | 130 | 13519 | 130 | 13519 | 130 | 13519 | 130 | 13519 | 130 |
| CAG promoter | 13523 | 1715 | 13523 | 1715 | 13523 | 1715 | 13523 | 1715 | 13523 | 1715 |
| CMVie | 13524 | 299 | 13524 | 299 | 13524 | 299 | 13524 | 299 | 13524 | 299 |
| CBA min. | 13525 | 283 | 13525 | 283 | 13525 | 283 | 13525 | 283 | 13525 | 283 |
| Signal | 13569 | 57 | 13569 | 57 | 13569 | 57 | 13569 | 57 | 13569 | 57 |
| Variable and/or | — | — | — | — | — | — | — | — | — | — |
| Constant | | | | | | | | | | |
| Linker | 1727 | 75 | 1727 | 75 | 1726 | 54 | 1726 | 54 | 1726 | 54 |
| Signal | 13567 | 57 | 13567 | 57 | 13570 | 57 | 13567 | 57 | 13567 | 57 |
| Variable and/or | — | — | — | — | — | — | — | — | — | — |
| Constant | | | | | | | | | | |
| PolyA | 13576 | 127 | 13576 | 127 | 13576 | 127 | 13576 | 127 | 13576 | 127 |
| 3′ ITR | 13521 | 130 | 13521 | 130 | 13521 | 130 | 13521 | 130 | 13521 | 130 |

In some embodiments, the AAV particle genome is VGC205 and comprises a 5′ inverted terminal repeat (ITR) sequence region and a 3′ITR sequence region, a CAG promoter comprising a CMVie region and a minimal CBA promoter region, a light chain signal, an antibody light chain variable and constant region, a F2A linker, a heavy chain signal, an antibody heavy chain variable and constant region and a polyadenylation signal sequence.

In some embodiments, the AAV particle genome is VGC206 and comprises a 5′ inverted terminal repeat (ITR) sequence region and a 3′ITR sequence region, a CAG promoter comprising a CMVie region and a minimal CBA promoter region, a light chain signal, an antibody light chain variable and constant region, a F2A linker, a heavy chain signal, an antibody heavy chain variable and constant region and a polyadenylation signal sequence.

In some embodiments, the AAV particle genome is VGC207 and comprises a 5′ inverted terminal repeat (ITR) sequence region and a 3′ ITR sequence region, a CAG promoter comprising a CMVie region and a minimal CBA promoter region, a light chain signal, an antibody light chain variable and constant region, a T2A linker, a heavy chain signal, an antibody heavy chain variable and constant region and a polyadenylation signal sequence.

In some embodiments, the AAV particle genome is VGC208 and comprises a 5′ inverted terminal repeat (ITR) sequence region and a 3′ITR sequence region, a CAG promoter comprising a CMVie region and a minimal CBA promoter region, a light chain signal, an antibody light chain variable and constant region, a T2A linker, a heavy chain signal, an antibody heavy chain variable and constant region and a polyadenylation signal sequence.

In some embodiments, the AAV particle genome is VGC209 and comprises a 5′ inverted terminal repeat (ITR) sequence region and a 3′ ITR sequence region, a CAG promoter comprising a CMVie region and a minimal CBA promoter region, a light chain signal, an antibody light chain variable and constant region, a T2A linker, a heavy chain signal, an antibody heavy chain variable and constant region and a polyadenylation signal sequence.

TABLE 82
Viral Genome Cassettes Sequence Regions

| | VGC210 | | VGC211 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| CAG Promoter | 13523 | 1715 | 13523 | 1715 |
| CMVie | 13524 | 299 | 13524 | 299 |
| CBA min. | 13525 | 283 | 13525 | 283 |
| Signal | 13569 | 57 | 13569 | 57 |
| Variable and/or Constant | — | — | — | — |
| Linker | 1724 | 12 | 1724 | 12 |
| Linker | 1726 | 54 | 1726 | 54 |
| Signal | 13567 | 57 | 13567 | 57 |
| Variable and/or Constant | — | — | — | — |
| PolyA | 13576 | 127 | 13576 | 127 |
| 3′ ITR | 13521 | 130 | 13521 | 130 |

in some embodiments, the AAV particle genome is VGC211 an comprises a 5′ inverted terminal repeat sequence region and a 3′ITR sequence region, a CAG promoter comprising a CMVie region and a minimal CBA promoter region, a light chain signal, an antibody light chain variable and constant region, a furin cleavage site, a T2A linker, a heavy chain signal, an antibody heavy chain variable and constant region and a polyadenylation signal sequence.

In some embodiments, the AAV particle genome is VGC212 and comprises a 5′ inverted terminal repeat (ITR) sequence region and a 3′ITR sequence region, a CAG promoter comprising a CMVie region and a minimal CBA promoter region, a light chain signal, an antibody light chain variable and constant region, a furin cleavage site, a T2A linker, a heavy chain signal, an antibody heavy chain variable and constant region and a polyadenylation signal sequence.

TABLE 83
Viral Genome Cassettes Sequence Regions

| | VGC212 | | VGC213 | | VGC214 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ITR | 13519 | 130 | 13519 | 130 | 13519 | 130 |
| CAG promoter | 13523 | 1715 | 13523 | 1715 | 13523 | 1715 |
| CMVie | 13524 | 299 | 13524 | 299 | 13524 | 299 |
| CBA min. | 13525 | 283 | 13525 | 283 | 13525 | 283 |
| Variable and/or | — | — | — | — | — | — |
| Constant | | | | | | |
| Linker | 13154 | 45 | 13154 | 45 | 13154 | 45 |
| Variable and/or | — | — | — | — | — | — |
| Constant | | | | | | |
| HA Tag | 13571 | 27 | 13571 | 27 | 13571 | 27 |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC212 and comprises a 5′ inverted terminal repeat (ITR) sequence region and a 3′ITR sequence region, a CAG promoter comprising a CMVie region and a minimal CBA promoter region, an antibody heavy chain variable region, a (G4S)3 linker, an antibody light chain variable region, a HA tag and a polyadenylation signal sequence.

In some embodiments, the AAV particle genome is VGC213 and comprises a 5′ inverted terminal repeat (ITR) sequence region and a 3′ ITR sequence region, a CAG promoter comprising a CMVie region and a minimal CBA promoter region, an antibody heavy chain variable region, a (G4S)3 linker, an antibody light chain variable region, a HA tag and a polyadenylation signal sequence.

In some embodiments, the AAV particle genome is VGC214 and comprises a 5′ inverted terminal repeat (ITR) sequence region and a 3′ITR sequence region, a CAG promoter comprising a CMVie region and a minimal CBA promoter region, an antibody heavy chain variable region, a (G4S)3 linker, an antibody light chain variable region, a HA tag and a polyadenylation signal sequence.

TABLE 84
Viral Genome Cassettes Sequence Regions

| | VGC215 | | VGC216 | | VGC217 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 136 | 13519 | 136 |
| GFAP | 13528 | 699 | 13528 | 699 | 13528 | 699 |
| Promoter | | | | | | |
| Intron | 13551 | 566 | 13551 | 566 | 13551 | 566 |
| Signal | 13567 | 57 | 13567 | 57 | 13567 | 57 |
| Variable and/or | — | — | — | — | — | — |
| Constant | | | | | | |
| Constant | 1742 | 969 | 1742 | 969 | 2243 | 291 |
| Linker | 1725 | 12 | 1724 | 12 | 1724 | 12 |
| Linker | 1727 | 75 | 1726 | 54 | 1726 | 54 |
| Signal | 1980 | 321 | 1980 | 321 | 1986 | 321 |
| Variable and/or | — | — | — | — | — | — |
| Constant | | | | | | |
| PolyA | 13576 | 127 | 13576 | 127 | 13576 | 127 |
| 3′ ITR | 13521 | 130 | 13521 | 136 | 13521 | 136 |

In some embodiments, the AAV particle genome is VGC215 and comprises a 5′ inverted terminal repeat (ITR) sequence region and a 3′ ITR sequence region, a GFAP promoter, a human beta-globin intron region, a heavy chain signal, an antibody heavy chain variable and constant region, a furin cleavage site, a F2A linker, a light chain signal, an antibody light chain variable and constant region and a polyadenylation signal sequence.

In some embodiments, the AAV particle genome is VGC216 and comprises a 5′ inverted terminal repeat (ITR) sequence region and a 3′ ITR sequence region, a GFAP promoter, a human beta-globin intron region, a heavy chain signal, an antibody heavy chain variable and constant region, a furin cleavage site, a T2A linker, alight chain signal, an antibody light chain variable and constant region and a polyadenylation signal sequence.

In some embodiments, the AAV particle genome is VGC217 and comprises a 5′ inverted terminal repeat (ITR) sequence region and a 3′ITR sequence region, a GFAP promoter, a human beta-globin intron region, a heavy chain signal, an antibody heavy chain variable and constant region, a furin cleavage site, a T2A linker, a light chain signal, an antibody light chain variable and constant region and a polyadenylation signal sequence.

TABLE 85
Viral Genome Cassettes Sequence Regions

| | VGC218 | | VGC219 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| GFAP Promoter | 13528 | 699 | 13528 | 699 |
| Intron | 13551 | 566 | 13551 | 566 |
| Signal | 13567 | 57 | 13567 | 57 |
| Variable and/or Constant | — | — | — | — |
| Hinge | 1737 | 198 | 1737 | 198 |
| Linker | 1725 | 12 | 1724 | 12 |
| Linker | 1727 | 75 | 1726 | 54 |
| Signal | 13569 | 57 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC218 and comprises a 5′ inverted terminal repeat (ITR) sequence region and a 3′ITR sequence region, a GFAP promoter, a human beta-globin intron region, a heavy chain signal, an antibody heavy chain variable and constant region, a hinge region, a furin cleavage site, a F2A linker, a light chain signal, an antibodylightchainvariableandconstantregionandaplyadenylationsignal sequence.

In some embodiments, the AAV particle genome is VGC219 and comprises a 5′ inverted terminal repeat (ITR) sequence region and a 3′ITR sequence region, a GFAP promoter, a human beta-globin intron region, a heavy chain signal, an antibody heavy chain variable and constant region, a hinge region, a furin cleavage site, a T2A linker, a light chain signal, an antibody light chain variable and constant region and a polyadenylation signal sequence.

TABLE 86
Viral Genome Cassettes Sequence Regions

| | VGC220 | | VGC221 | | VGC222 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 138 | 13519 | 130 | 13519 | 130 |
| GFAP | 13528 | 699 | 13528 | 699 | 13528 | 699 |
| Promoter | | | | | | |
| Intron | 13551 | 568 | 13551 | 566 | 13551 | 566 |
| Signal | 13569 | 57 | 13569 | 57 | 13569 | 57 |
| Variable and/or | — | — | — | — | — | — |
| Constant | | | | | | |
| Linker | 13153 | 38 | 13154 | 45 | 13155 | 60 |
| Variable and/or | — | — | — | — | — | — |
| Constant | | | | | | |
| HA Tag | 13571 | 27 | 13571 | 27 | 13571 | 27 |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC220 and comprises a 5′ inverted terminal repeat (ITR) sequence region and a 3′ ITR sequence region, a GFAP promoter, a human beta-globin intron region, a light chain signal, an antibody light chain variable region, a (G4S)2 linker, an antibody heavy chain variable region, a HA tag and a polyadenylation signal sequence.

In some embodiments, the AAV particle genome is VGC221 and comprises a 5′ inverted terminal repeat (ITR) sequence region and a 3′ITR sequence region, a GFAP promoter, a human beta-globin intron region, a light chain signal, an antibody light chain variable region, a (G4S)3 linker, an antibody heavy chain variable region, a HA tag and a polyadenylation signal sequence.

In some embodiments, the AAV particle genome is VGC222 and comprises a 5′ inverted terminal repeat (ITR) sequence region and a 3′ ITR sequence region, a GFAP promoter, a human beta-globin intron region, alight chain signal, an antibody light chain variable region, a (G4S)4 linker, an antibody heavy chain variable region, a HA tag and a polyadenylation signal sequence.

TABLE 87
Viral Genome Cassettes Sequence Regions

| | VGC223 | | VGC224 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| GFAP Promoter | 13528 | 699 | 13528 | 699 |
| Intron | 13551 | 566 | 13551 | 566 |
| Signal | 13569 | 57 | 13569 | 57 |
| Variable and/or Constant | — | — | — | — |
| Linker | 1727 | 75 | 1826 | 54 |
| Signal | 13567 | 57 | 13570 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC223 and comprises a 5′ inverted terminal repeat (ITR) sequence region and a 3′ITR sequence region, a GFAP promoter, a human beta-globin intron region, a light chain signal, an antibody light chain variable and constant region, a F2A linker, a heavy chain signal, an antibody heavy chain variable and constant region and a polyadenylation signal sequence.

In some embodiments, the AAV particle genome is VGC224 and comprises a 5′ inverted terminal repeat (ITR) sequence region and a 3′ ITR sequence region, a GFAP promoter, a human beta-globin intron region, a light chain signal, an antibody light chain variable and constant region, a T2A linker, a heavy chain signal, an antibody heavy chain variable and constant region and a polyadenylation signal sequence.

TABLE 88
Viral Genome Cassettes Sequence Regions

| | VGC225 | | VGC226 | | VGC227 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 130 | 13519 | 130 |
| SYN Promoter | 13529 | 557 | 13529 | 557 | 13529 | 557 |
| intron | 13551 | 566 | 13551 | 566 | 13551 | 566 |
| Signal | 13567 | 57 | 13567 | 57 | 13567 | 57 |
| Variable and/or | — | — | — | — | — | — |
| Constant | | | | | | |
| Linker | 1725 | 12 | 1724 | 12 | 1724 | 12 |
| Linker | 1727 | 75 | 1726 | 54 | 1726 | 54 |
| Signal | 13569 | 57 | 13569 | 57 | 13569 | 57 |
| Variable and/or | — | — | — | — | — | — |
| Constant | | | | | | |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC225 and comprises a 5′ inverted terminal repeat (ITR) sequence region and a 3′ ITR sequence region, a synapsin promoter, a human beta-globin intron region, a heavy chain signal, an antibody heavy chain variable and constant region, a furin cleavage site, a F2A linker, alight chain signal, an antibody light chain variable and constant region and a polyadenylation signal sequence.

In some embodiments, the AAV particle genome is VGC226 and comprises a 5′ inverted terminal repeat (ITR) sequence region and a 3′ ITR sequence region, a synapsin promoter, a human beta-globin intron region, a heavy chain signal, an antibody heavy chain variable and constant region, a furin cleavage site, a F2A linker, alight chain signal, an antibody light chain variable and constant region and a polyadenylation signal sequence.

In some embodiments, the AAV particle genome is VGC227 and comprises a 5′ inverted terminal repeat (ITR) sequence region and a 3′ ITR sequence region, a synapsin promoter, a human beta-globin intron region, a heavy chain signal, an antibody heavy chain variable and constant region, a furin cleavage site, a F2A linker, alight chain signal, an antibody light chain variable and constant region and a polyadenylation signal sequence.

TABLE 89
Viral Genome Cassettes Sequence Regions

| | VGC228 | | VGC229 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| SYN Promoter | 13529 | 557 | 13529 | 557 |
| Intron | 13551 | 566 | 13551 | 566 |
| Signal | 13567 | 57 | 13567 | 57 |
| Variable and/or Constant | — | — | — | — |
| Linker | 1737 | 198 | 1737 | 198 |
| Linker | 1724 | 12 | 1724 | 12 |
| Linker | 1727 | 75 | 1726 | 54 |
| Signal | 13569 | 54 | 13569 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC228 and comprises a 5′ inverted terminal repeat (ITR) sequence region and a 3′ ITR sequence region, a synapsin promoter, a human beta-globin intron region, a heavy chain signal, an antibody heavy chain variable and constant region, a hinge region, a furin cleavage site, a F2A linker, alight chain signal, an antibody light chain variable and constant region and a polyadenylation signal sequence.

In some embodiments, the AAV particle genome is VGC229 and comprises a 5′ inverted terminal repeat (ITR) sequence region and a 3′ ITR sequence region, a synapsin promoter, a human beta-globin intron region, a heavy chain signal, an antibody heavy chain variable and constant region, a hinge region, a furin cleavage site, a T2A linker, alight chain signal, an antibody light chain variable and constant region and a polyadenylation signal sequence.

TABLE 90
Viral Genome Cassettes Sequence Regions

| | VGC230 | | VGC231 | | VGC232 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 130 | 13519 | 130 |
| SYN Promoter | 13529 | 557 | 13529 | 557 | 13529 | 557 |
| Intron | 13551 | 566 | 13551 | 566 | 13551 | 566 |
| Signal | 13569 | 57 | 13569 | 57 | 13569 | 57 |
| Variable and/or | — | — | — | — | — | — |
| Constant | | | | | | |
| Linker | 13153 | 30 | 13154 | 45 | 13155 | 60 |
| Variable and/or | — | — | — | — | — | — |
| Constant | | | | | | |
| HA Tag | 13571 | 27 | 13571 | 27 | 13571 | 27 |
| PolyA | 13576 | 127 | 13576 | 127 | 13576 | 127 |
| 3′ ITR | 13521 | 130 | 13521 | 130 | 13521 | 130 |

In some embodiments, the AAV particle genome is VGC230 and comprises a 5′ inverted terminal repeat (ITR) sequence region and a 3′ ITR sequence region, a synapsin promoter, a human beta-globin intron region, a light chain signal, an antibody light chain variable region, a (G4S)2 linker, an antibody heavy chain variable region, a HA tag and a polyadenylation signal sequence.

In some embodiments, the AAV particle genome is VGC231 and comprises a 5′ inverted terminal repeat (ITR) sequence region and a 3′ ITR sequence region, a synapsin promoter, a human beta-globin intron region, alight chain signal, an antibody light chain variable region, a (G4S)3 linker, an antibody heavy chain variable region, a HA tag and a polyadenylation signal sequence.

In some embodiments, the AAV particle genome is VGC232 and comprises a 5′ inverted terminal repeat (ITR) sequence region and a 3′ ITR sequence region, a synapsin promoter, a human beta-globin intron region, a light chain signal, an antibody light chain variable region, a (G4S)4 linker, an antibody heavy chain variable region, a HA tag and a polyadenylation signal sequence.

TABLE 91
Viral Genome Cassettes Sequence Regions

| | VGC232 | | VGC233 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| SYN Promoter | 13529 | 557 | 13529 | 557 |
| Intron | 13551 | 566 | 13551 | 566 |
| Signal | 13569 | 57 | 13569 | 57 |
| Variable and/or Constant | — | — | — | — |
| Linker | 1727 | 75 | 1726 | 54 |
| Signal | 13567 | 57 | 13570 | 57 |
| Variable and/or Constant | — | — | — | — |
| PolyA | 13576 | 127 | 13576 | 127 |
| 3′ ITR | 13521 | 130 | 13521 | 130 |

In some embodiments, the AAV particle genome is VGC233 and comprises a 5′ inverted terminal repeat (ITR) sequence region and a 3′ITR sequence region, a synapsin promoter, a human beta-globin intron region, a light chain signal, an antibody light chain variable region, a F2A linker, a heavy chain signal, an antibody heavy chain variable and constant region and a polyadenylation signal sequence.

In some embodiments, the AAV particle genome is VGC234 and comprises a 5′ inverted terminal repeat (ITR) sequence region and a 3′ITR sequence region, a synapsin promoter, a human beta-globin intron region, a light chain signal, an antibody light chain variable region, a T2A linker, a heavy chain signal, an antibody heavy chain variable and constant region and a polyadenylation signal sequence.

TABLE 92
Viral Genome Cassettes Sequence Regions

| | VGC235 | | VGC236 | | VGC237 | |
|---|---|---|---|---|---|---|
| | Region | | Region | | Region | |
| Sequence | SEQ ID | Region | SEQ ID | Region | SEQ ID | Region |
| Regions | NO | length | NO | length | NO | length |
| 5′ ITR | 13519 | 130 | 13519 | 136 | 13519 | 130 |
| CMVie | 13534 | 386 | 13534 | 386 | 13534 | 380 |
| CB Promoter | 13526 | 266 | 13526 | 266 | 13526 | 260 |
| Intron | 13551 | 566 | 13551 | 566 | 13551 | 566 |
| Signal | 13567 | 57 | 13567 | 57 | 13567 | 57 |
| Variable and/or | — | — | — | — | — | — |
| Constant | | | | | | |
| Linker | 1725 | 12 | 1724 | 12 | 1724 | 12 |
| Linker | 1727 | 75 | 1726 | 54 | 1726 | 54 |
| Signal | 13569 | 57 | 13569 | 57 | 13569 | 57 |
| Variable and/or | — | — | — | — | — | — |
| Constant | | | | | | |
| PolyA | 13576 | 127 | 13576 | 127 | 13576 | 127 |
| 3′ ITR | 13521 | 130 | 13521 | 130 | 13521 | 136 |

In some embodiments, the AAV particle genome is VGC235 and comprises a 5′ inverted terminal repeat (ITR) sequence region and a 3′ITR sequence region, a CMVie region and a CB promoter, a human beta-globin intron region, a heavy chain signal, an antibody heavy chain variable and constant region, a furin cleavage site, a F2A linker, alight chain signal, an antibody light chain variable and constant region and a polyadenylation signal sequence.

In some embodiments, the AAV particle genome is VGC236 and comprises a 5′ inverted terminal repeat (ITR) sequence region and a 3′ ITR sequence region, a CMVie region and a CB promoter, a human beta-globin intron region, a heavy chain signal, an antibody heavy chain variable and constant region, a furin cleavage site, a T2A linker, a light chain signal, an antibody light chain variable and constant region and a polyadenylation signal sequence.

In some embodiments, the AAV particle genome is VGC237 and comprises a 5′ inverted terminal repeat (ITR) sequence region and a 3′ITR sequence region, a CMVie region and a CB promoter, a human beta-globin intron region, a heavy chain signal, an antibody heavy chain variable and constant region, a furin cleavage site, a T2A linker, alight chain signal, an antibody light chain variable and constant region and a polyadenylation signal sequence.

TABLE 93
Viral Genome Cassettes Sequence Regions

| | VGC238 | | VGC239 | |
|---|---|---|---|---|
| | Region | Region | Region | Region |
| Sequence Regions | SEQ ID NO | length | SEQ ID NO | length |
| 5′ ITR | 13519 | 130 | 13519 | 130 |
| CMVie | 13534 | 380 | 13534 | 380 |
| CB Promoter | 13526 | 260 | 13526 | 260 |
| Intron | 13551 | 566 | 13551 | 566 |
| Signal | 13569 | 57 | 13569 | 57 |
| Variable and/or Constant | — | — | — | — |
| Linker | 1727 | 75 | 1726 | 54 |
| Signal | 13567 | 57 | 13570 | 57 |
| Variable and/or Constant | — | — | — | — |
| PolyA | 13576 | 127 | 13576 | 127 |
| 3′ ITR | 13521 | 130 | 13521 | 130 |

in some embodiments, the AAV particle genome is an comprises a 5′ inverted terminal repeat sequence region and a 3′ ITR sequence region, a CMVie region and a CB promoter, a human beta-globin intron region, a light chain signal, an antibody light chain variable and constant region, a F2A linker, a heavy chain signal, an antibody heavy chain variable and constant region and a polyadenylation signal sequence.

In some embodiments, the AAV particle genome is VGC239 and comprises a 5′ inverted terminal repeat (ITR) sequence region and a 3′ITR sequence region, a CMVie region and a CB promoter, a human beta-globin intron region, alight chain signal, an antibody light chain variable and constant region, a F2A linker, a heavy chain signal, an antibody heavy chain variable and constant region and a polyadenylation signal sequence.

### II. Formulation and Delivery

### Pharmaceutical Compositions

According to the present disclosure the AAV particles may be prepared as pharmaceutical compositions. It will be understood that such compositions necessarily comprise one or more active ingredients and, most often, a pharmaceutically acceptable excipient.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the AAV particle pharmaceutical compositions described herein may comprise at least one payload. As a non-limiting example, the pharmaceutical compositions may contain an AAV particle with 1, 2, 3, 4 or 5 payloads. In some embodiments, the pharmaceutical composition may contain a nucleic acid encoding a payload construct encoding proteins selected from antibodies and/or antibody-based compositions.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects.

### Formulations

The AAV particles of the disclosure can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein and/or (7) allow for regulatable expression of the payload.

Formulations of the present disclosure can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an AAV particle carrying a payload region encoding the polypeptides of the disclosure or to the antibody or antibody-based composition encoded by a viral genome of by an AAV particle as described herein.

Formulations of the AAV particles and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the AAV particles of the disclosure may be formulated in PBS with 0.001% of pluronic acid (F-68) at a pH of about 7.0.

Relative amounts of the active ingredient (e.g. AV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the AAV formulations described herein may contain sufficient AAV particles for expression of at least one expressed functional antibody or antibody-based composition. As a non-limiting example, the AAV particles may contain viral genomes encoding 1, 2, 3, 4 or 5 functional antibodies.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137.140; the content of which is incorporated herein by reference in its entirety).

### Excipients and Diluents

The AAV particles of the disclosure can be formulated using one or more excipients or diluents to (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the polypeptides of the disclosure.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, AAV particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

In some embodiments, the AAV particle pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha-lonone; Almond Oil; Alpha-Terpineol; Alpha-Tocopherol; Alpha-Tocopherol Acetate, DI-; Alpha-Tocopherol, DI-; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, DI-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, DI-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287: Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No, 4; Fd&C Red No, 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig. 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O FI-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, DI-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, DI-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono and Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene. 1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 4513; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg. 120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90 g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride):Sebacic Acid; Poly(Dimethylsiloxane/Methylvinylsiloxane/Methylhydrogensiloxane) Dimethylvinyl Or Dimethylhydroxy Or Trimethyl Endblocked; Poly(DI-Lactic-Co-Glycolic Acid), (50:50; Poly(DI-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q74301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, DI-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations of AAV particles disclosed herein may include cations or anions. In some embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydrochloride, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### III. Administration and Dosing

### Administration

The AAV particles of the present disclosure may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into the substance of a tissue, e.g., brain tissue), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, ganglionic (to the ganglion), intraganglionic (within the ganglion), and/or spinal.

In some embodiments, compositions may be administered in a way which allows them to cross the blood-brain barrier, vascular barrier, or other epithelial barrier. The AAV particles of the present disclosure may be administered in any suitable form, either as a liquid solution or suspension, as a solid form suitable for liquid solution or suspension in a liquid solution. The AAV particles may be formulated with any appropriate and pharmaceutically acceptable excipient.

In some embodiments, the AAV particles of the present disclosure may be delivered to a subject via a single route administration.

In some embodiments, the AAV particles of the present disclosure may be delivered to a subject via a multi-site route of administration. A subject may be administered at 2, 3, 4, 5 or more than 5 sites.

In some embodiments, a subject may be administered the AAV particles of the present disclosure using a bolus infusion.

In some embodiments, a subject may be administered the AAV particles of the present disclosure using sustained delivery over a period of minutes, hours or days. The infusion rate may be changed depending on the subject, distribution, formulation or another delivery parameter.

In some embodiments, the AAV particles of the present disclosure may be delivered by intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, the AAV particles of the present disclosure may be delivered by oral administration. Non-limiting examples of oral administration include a digestive tract administration and a buccal administration.

In some embodiments, the AAV particles of the present disclosure may be delivered by intraocular delivery route. A non-limiting example of intraocular administration include an intravitreal injection.

In some embodiments, the AAV particles of the present disclosure may be delivered by intranasal delivery route. Non-limiting examples of intranasal delivery include administration of nasal drops or nasal sprays.

In some embodiments, the AAV particles that may be administered to a subject by peripheral injections. Non-limiting examples of peripheral injections include intraperitoneal, intramuscular, intravenous, conjunctival or joint injection. It was disclosed in the art that the peripheral administration of AAV vectors can be transported to the central nervous system, for example, to the motor neurons (e.g., U. S. Patent Publication Nos. 20100240739; and 20100130594; the content of each of which is incorporated herein by reference in their entirety).

In some embodiments, the AAV particles may be delivered by injection into the CSF pathway. Non-limiting examples of delivery to the CSF pathway include intrathecal and intracerebroventricular administration.

In some embodiments, the AAV particles may be delivered by systemic delivery. As a non-limiting example, the systemic delivery may be by intravascular administration.

In some embodiments, the AAV particles of the present disclosure may be administered to a subject by intracranial delivery (See, e.g., U.S. Pat. No. 8,119,611; the content of which is incorporated herein by reference in its entirety).

In some embodiments, the AAV particles of the present disclosure may be administered to a subject by intraparenchymal administration.

In some embodiments, the AAV particles of the present disclosure may be administered to a subject by intramuscular administration.

In some embodiments, the AAV particles of the present disclosure are administered to a subject and transduce muscle of a subject. As a non-limiting example, the AAV particles are administered by intramuscular administration.

In some embodiments, the AAV particles of the present disclosure may be administered to a subject by intravenous administration.

In some embodiments, the AAV particles of the present disclosure may be administered to a subject by subcutaneous administration.

In some embodiments, the AAV particles of the present disclosure may be administered to a subject by topical administration.

In some embodiments, the AAV particles may be delivered by direct injection into the brain. As a non-limiting example, the brain delivery may be by intrastriatal administration.

In some embodiments, the AAV particles may be delivered by more than one route of administration. As non-limiting examples of combination administrations, AAV particles may be delivered by intrathecal and intracerebroventricular, or by intravenous and intraparenchymal administration.

### Parenteral and Injectable Administration

In some embodiments, pharmaceutical compositions, AAV particles of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR*, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

In some embodiments, pharmaceutical compositions, AAV particles of the present disclosure may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

In some embodiments, pharmaceutical compositions, AAV particles of the present disclosure may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, pharmaceutical compositions, AAV particles of the present disclosure may be formulated for administration topically. The skin may be an ideal target site for delivery as it is readily accessible. Three routes are commonly considered to deliver pharmaceutical compositions, AAV particles of the present disclosure to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Pharmaceutical compositions, AAV particles of the present disclosure can be delivered to the skin by several different approaches known in the art.

In some embodiments, the disclosure provides for a variety of dressings (e.g., wound dressings) or bandages (e.g., adhesive bandages) for conveniently and/or effectively carrying out methods of the present disclosure. Typically dressing or bandages may comprise sufficient amounts of pharmaceutical compositions, AAV particles of the present disclosure described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of pharmaceutical compositions, AAV particles of the present disclosure to the body. Such dosage forms may be prepared, for example, by dissolving and/or dispensing pharmaceutical compositions, AAV particles in the proper medium. Alternatively, or additionally, rates may be controlled by either providing rate controlling membranes and/or by dispersing pharmaceutical compositions, AAV particles in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, pharmaceutical compositions, AAV particles of the present disclosure are formulated in depots for extended release. Generally, specific organs or tissues (“target tissues”) are targeted for administration.

In some aspects of the disclosure, pharmaceutical compositions, AAV particles of the present disclosure are spatially retained within or proximal to target tissues. Provided are methods of providing pharmaceutical compositions, AAV particles, to target tissues of mammalian subjects by contacting target tissues (which comprise one or more target cells) with pharmaceutical compositions, AAV particles, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particles, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions, AAV particles, administered to subjects are present intracellularly at a period of time following administration. For example, intramuscular injection to mammalian subjects may be performed using aqueous compositions comprising pharmaceutical compositions, AAV particles of the present disclosure and one or more transfection reagents, and retention is determined by measuring the amount of pharmaceutical compositions, AAV particles, present in muscle cells.

Certain aspects of the disclosure are directed to methods of providing pharmaceutical compositions, AAV particles of the present disclosure to a target tissues of mammalian subjects, by contacting target tissues (comprising one or more target cells) with pharmaceutical compositions, AAV particles under conditions such that they are substantially retained in such target tissues. Pharmaceutical compositions, AAV particles comprise enough active ingredient such that the effect of interest is produced in at least one target cell. In some embodiments, pharmaceutical compositions, AAV particles generally comprise one or more cell penetration agents, although “naked” formulations (such as without cell penetration agents or other agents) are also contemplated, with or without pharmaceutically acceptable carriers.

### Pulmonary Administration

In some embodiments, pharmaceutical compositions, AAV particles of the present disclosure may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

In some embodiments, pharmaceutical compositions, AAV particles of the present disclosure may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### Ophthalmic or Otic Administration

In some embodiments, pharmaceutical compositions, AAV particles of the present disclosure may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

### Delivery

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

The present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

### Dose and Regimen

The present disclosure provides methods of administering AAV particles in accordance with the disclosure to a subject in need thereof. The pharmaceutical, diagnostic, or prophylactic AAV particles and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific AAV particle employed; the duration of the treatment; drugs used in combination or coincidental with the specific AAV particle employed; and like factors well known in the medical arts.

In certain embodiments, AAV particle pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In certain embodiments, AAV particle pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, AAV particles may be administered at 50 μl/site and/or 150 μl/site.

The desired dosage of the AAV particles of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the AAV particles of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24 hour period, may be administered by any of these methods, or as a combination of these methods, or by any other methods suitable for a pharmaceutical administration.

In some embodiments, delivery of the AAV particles of the present disclosure to a subject provides neutralizing activity to a subject. The neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, delivery of the AAV particles of the present disclosure results in minimal serious adverse events (SAEs) as a result of the delivery of the AAV particles.

In some embodiments, delivery of AAV particles to cells of the central nervous system (e.g., parenchyma) may comprise a total dose between about 1×10⁶ VG and about 1×10⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10³, 3×10⁸, 4×10⁸, 5×10³, 6×10⁸, 7×10⁸, 8×10, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10⁹, 5×10⁹, 6×10¹⁰, 7×10¹⁰, 8×10⁹, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10², 5×10¹², 6×10², 7×10¹², 8×10², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶VG. As a non-limiting example, the total dose is 1×10¹³VG. As another non-limiting example, the total dose is 2.1×10¹²VG.

In some embodiments, delivery of AAV particles to cells of the central nervous system (e.g., parenchyma) may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶′VG/mL In some embodiments, the delivery comprises a composition concentration of 1×10¹³VG/mL. In some embodiments, the delivery comprises a composition concentration of 2.1×10¹² VG/mL.

### Combinations

The AAV particles may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### Measurement of Expression

Expression of payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

### Bioavailability

The AAV particles, when formulated into a composition with a delivery agent as described herein, can exhibit an increase in bioavailability as compared to a composition lacking a delivery agent as described herein. As used herein, the term “bioavailability” refers to the systemic availability of a given amount of AAV particle or expressed payload administered to a mammal. Bioavailability can be assessed by measuring the area under the curve (AUC) or the maximum serum or plasma concentration (Cₘₐₓ) of the composition following. AUC is a determination of the area under the curve plotting the serum or plasma concentration of a compound (e.g., AAV particles or expressed payloads) along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in its entirety.

The Cₘₐₓ value is the maximum concentration of the AAV particle or expressed payload achieved in the serum or plasma of a mammal following administration of the AAV particle to the mammal. The Cₘₐₓ value of can be measured using methods known to those of ordinary skill in the art. The phrases “increasing bioavailability” or “improving the pharmacokinetics,” as used herein mean that the systemic availability of a first AAV particle or expressed payload, measured as AUC, Cₘₐₓ, or Cₘᵢₙ in a mammal is greater, when co-administered with a delivery agent as described herein, than when such co-administration does not take place. In some embodiments, the bioavailability can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

As used herein “therapeutic window” refers to the range of plasma concentrations, or the range of levels of therapeutically active substance at the site of action, with a high probability of eliciting a therapeutic effect. In some embodiments, the therapeutic window of the AAV particle as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

As used herein, the term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} a can be used to determine a loading dose to achieve a steady state concentration. In some embodiments, the volume of distribution of the AAV particles as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In some embodiments, the biological effect of the AAV particles delivered to the animals may be categorized by analyzing the payload expression in the animals. The payload expression may be determined from analyzing a biological sample collected from a mammal administered the AAV particles of the present disclosure. For example, a protein expression of 50-200 pg/ml for the protein encoded by the AAV particles delivered to the mammal may be seen as a therapeutically effective amount of protein in the mammal.

### IV. Methods and Uses of the Compositions of the Disclosure

The present disclosure provides a method for treating a disease, disorder and/or condition in a mammalian subject, including a human subject, comprising administering to the subject any of the AAV particles described herein or administering to the subject any of the described compositions, including pharmaceutical compositions, described herein.

In some embodiments, the AAV particles of the present disclosure are administered to a subject prophylactically.

In some embodiments, the AAV particles of the present disclosure are administered to a subject having at least one of the diseases described herein.

In some embodiments, the AAV particles of the present disclosure are administered to a subject to treat a disease or disorder described herein. The subject may have the disease or disorder or may be at-risk to developing the disease or disorder.

In some embodiments, the AAV particles of the present disclosure are part of an active immunization strategy to protect against diseases and disorders. In an active immunization strategy, a vaccine or AAV particles are administered to a subject to prevent an infectious disease by activating the subject's production of antibodies that can fight off invading bacteria or viruses.

In some embodiments, the AAV particles of the present disclosure are part of a passive immunization strategy. In a passive immunization strategy, antibodies against a particular infectious agent are given directly to the subject.

### Therapeutic Applications: Infectious Disease

AAV particles and methods of using the AAV particles described in the present disclosure may be used to prevent, manage and/or treat infectious disease. As a non-limiting example, the AAV particles of the present disclosure comprise a nucleic acid sequence encoding at least one of the sequences described in Table 3 (SEQ ID NO: 1740-2141).

The methods, components and compositions of the present disclosure may be used to diagnose, prevent, treat and/or manage infectious diseases. As used herein, the term “Infectious disease” may refer to any disorder and/or condition caused by invasion into the body of an exogenous organism or infection agent that is not typically present such as, but not limited to, viruses, bacteria, prions, nematodes, fungus, parasites or arthropods. Infectious diseases are also known as transmissible diseases or communicable diseases. Infectious diseases and/or infection related diseases, disorders, and/or conditions that may be treated by methods, components and compositions of the present disclosure include, but are not limited to, Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia. Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis—(Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium Difficile Infection, ClostriDium Perfringens (Epsilon Toxin), Coccidioidomycosis fungal infection (Valley fever; desert rheumatism), Coenurosis, Colorado tick fever, Condyloma accuminata, Condyloma accuminata (Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin-B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fasciollasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome-Perihepatitis, Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes-genital, Herpes labialis, Herpes-neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichlosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis-Swimming Pool conjunctivitis-Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease-lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping III, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Monillasis, monkeypox, Mononucleosis, Mononucleosis-like syndrome, Montezuma's Revenge, Morbilli, MRSA (methicillin-resistant Staphylococcus aureus) infection, Mucormycosis. Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers. Buruli ulcers, Mycotic vulvovaginitis, Myositis, Nanukayami fever, Necrotizing fasciitis, Necrotizing fasciitis-Type 1, Necrotizing fasciitis. Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra(Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans., Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium Tetani, Bordetella pertussis, Bordetella paratussis, Mycobacterium, Francisella Tularensis, Toxoplasma gondii, Candida (C. albicans, C. glabrata, C. parapsilosis, C. tropicalis, C. krusei and C. lusitaniae) and/or any other infectious diseases, disorders or syndromes.

Additionally, an infection or symptoms associated with an infection may be caused by one or more toxins produced by such agents. Humans, and other mammals, react to infections with an innate immune system response, often involving an inflammation. The illnesses and symptoms involved with infections vary according to the infectious agent. Many infections may be subclinical without presenting any definite or observable symptoms, whereas some infections cause severe symptoms, require hospitalization or may be life-threatening. Some infections are localized, whereas some may overcome the body through blood circulation or lymphatic vessels. Some infections have long-term effects on wellbeing of infected individuals.

Infectious agents may be transmitted to humans via different routes. For example, infection agents may be transmitted by direct contact with an infected human, an infected animal, or an infected surface. Infections may be transmitted by direct contact with bodily fluids of an infected human or an animal, e.g. blood, saliva, sweat, tears, mucus, female ejaculate, semen, vomit or urine. For example, infection may be transmitted by a fecal-oral route, referring to an infected person shedding the virus in fecal particles which then enters to person's mouth causing infection. The fecal-oral route is especially common transmission route in environments with poor sanitation and hygiene. Non-limiting examples of agents transmitted by the fecal-oral route include bacteria, e.g. shigella, Salmonella typhi and Vibrio cholerae, virus, e.g. norovirus, rotavirus, enteroviruses, and hepatitis A, fungi, protozoans e.g. Entamoeba histolytica, parasites, tape worms, transmitted by contaminated food or beverage, leading to food poisoning or gastroenteritis. Infections may be transmitted by a respiratory route, referring to agents that are spread through the air. Typical examples include agents spread as small droplets of liquid or as aerosols, e.g. respiratory droplets expelled from the mouth and nose while coughing and sneezing. Typical examples of respiratory transmitted diseases include the common cold mostly implicated to rhinoviruses, influenza caused by influenza viruses, respiratory tract infections caused by e.g. respiratory syncytial virus (RSV). Infections may be transmitted by a sexual transmission route. Examples of common sexually transmitted infections include e.g. human immunodeficiency virus (HIV) causing acquired immune deficiency syndrome (AIDS), chlamydia caused by Neisseria gonorrhoeae bacteria, fungal infection Candidiasis caused by Candida yeast, and Herpes Simplex disease caused by herpes simplex virus. Infections may be transmitted by an oral transmission route, e.g. by kissing or sharing a drinking glass. A common infection transmitted by oral transmission is an infectious mononucleosis caused by Epstein-Barr virus. Infections may be transmitted by a vertical transmission, also known as “mother-to-child transmission,” from mother to an embryo, fetus or infant during pregnancy or childbirth. Examples of infection agents that may be transmitted vertically include HIV, chlamydia, rubella, Toxoplasma gondii, and herpes simplex virus. Infections may be transmitted by an iatrogenic route, referring to a transmission by medical procedures such as injection (contaminated reused needles and syringes), or transplantation of infected material, blood transfusions, or infection occurring during surgery. For example, methicillin-resistant Staphylococcus aureus (MRSA), which may cause several severe infections, may be transmitted via iatrogenic route during surgery. Infections may also be transmitted by vector-borne transmission, where a vector may be an organism transferring the infection agents from one host to another. Such vectors may be triatomine bugs, e.g. trypanosomes, parasites, animals, arthropods including e.g. mosquitos, flies, lice, flees, tick and mites or humans. Non-limiting examples of mosquito-borne infections include Dengue fever, West Nile virus related infections, Yellow fever and Chikungunya fever. Non-limiting examples of parasite-borne diseases include malaria, Human African trypanosomiasis and Lyme disease. Non-limiting examples of diseases spread by humans or mammals include HIV, Ebola hemorrhagic fever and Marburg fever.

Traditionally infectious diseases are treated with medications and/or good supportive care. Medical prevention, treatment and/or management of bacterial infections may include administration of antibiotics. Antibiotics may inhibit the colonization of bacteria or kill the bacteria. Antibiotics include e.g. penicillins, cephalosporins, macrolides, fluoroquinolones, sulfonamides, tetracyclines, and aminoglycosides. Antibiotics may be specific to a certain bacteria or act against broad spectrum of bacteria. Some types of bacteria are especially susceptible to antibiotics, whereas some bacteria are more resistant. Development of bacterial strain mutations that are resistant to antibiotics is an increasing concern. Methicillin-resistant Staphylococcus aureus (MRSA), vancomycin-resistant Enterococcus (VRE), multi-drug-resistant Mycobacterium tuberculosis (MDR-TB) and Klebsiella pneumoniae carbapenemase-producing bacteria (KPC) are examples of bacteria that are resistant to most general antibiotics. Due to the emerging resistance, unnecessary administration and overdosing of antibiotics should be avoided. Medical prevention, treatment and/or management of viral infections may include administration of antiviral medications. Antiviral medications may be specific to a certain bacteria or act against a broad spectrum of viruses. Currently antiviral medications are available for e.g. HIV, influenza, hepatitis B and C. Medical prevention, treatment and/or management of viral infections may include administration of antifungal medication. Antifungal medication kills or prevents the growth of fungi. Types of antifungal medications include e.g. imidazoles, triazoles and thiazoles, allylamines, and echinocandins. Development of antifungal medication capable of targeting fungal cells without affecting human cells is a challenge due to the similarities of human and fungal cell on the molecular level. Typically, medical treatment is combined with good supportive care, which includes provision of fluids, bed rest, medication to relieve pain and lower fever, supportive alternative medicine such as vitamins, antioxidants and other supplements important for wellbeing of patients.

Antibody therapies for infectious diseases have also been developed. Examples of commercial therapeutic antibodies include raxibacumab (developed by Cambridge Antibody Technology and Human Genome Sciences) which is an antibody for the prophylaxis and treatment of inhaled anthrax, SHIGAMAB™ (developed by Bellus Health Inc.) is a monoclonal antibody for treatment of Shiga toxin induced hemolytic uremic syndrome, and actoxumab and bezlotoxumab (developed by Medarex Inc. and the University of Massachusetts Medical School) are commercial human monoclonal antibodies targeting C. difficile toxin A and toxin B, respectively.

### Common Infectious Diseases

### John Cunningham Virus (JCV)

John Cunningham Virus is a common human polyomavirus. The transmission route of JCV is unknown. The virus is suspected to be spread by contaminated water and may be obtained through tonsils or by the gastrointestinal tract 70-90% of humans are estimated to be infected by the virus, and for normal healthy individuals the infection is asymptomatic. However, for patients with weakened immune system, JCV may lead to Progressive multifocal leukoencephalopathy (PML). PML is a condition characterized by multifocal progressive damage or inflammation of the white matter of the brain. The symptoms include clumsiness, progressive weakness and changes in visual, speech and personality. PLM has a mortality rate of 30-50% and patients who survive the disease are left with severe neurological disabilities PML occurs in patients with a severe immunodeficiency, most commonly in patients with HIV/AIDS. As many as 5% of HIV/AIDS patients are affected by PML. Individuals with other autoimmune conditions such as multiple sclerosis, rheumatoid arthritis, and systemic lupus erythematosus are also at risk, as well as individuals going through immunosuppressive therapy for cancer, e.g. lymphoma or Hodgkin's disease, or organ transplant. PML associated with immunosuppressive therapy is an increasing concern. For example, commercial antibody natalizumab (TYSABRI®, developed by Biogen Idec) for treatment of multiple sclerosis increases susceptibility to PML. Other drugs associated with increased risk of PML include Rituximab (RITUXAN®, developed by IDEC Pharmaceuticals), Efalizumab (RAPTIVA® developed by Genentech and XOMA) and Mycophenolate mofetil (CELLCEPT®, developed by Genentech).

JCV is a nonenveloped, T=7 icosahedral virus with a closed circular, double-stranded DNA genome. The major capsid component is the viral protein VP1 is made of 72 pentamers formed by VP1 monomers linked through the C terminal end. VP1 starts the infection by binding to the receptor target cells. After initial infection, typically occurring in childhood or adolescence, the virus stays quiescent in the kidneys and the lymphoid organs. In healthy individuals, the virus may replicate in kidney without causing any symptoms. However, in patients with weakened immune system, JCV may cross the blood-brain barrier into the central nervous system causing PML.

As of today, there is no known cure for PML. Current therapies focus on reversing the immune deficiency to slow down or stop the progress of the disease. There remains a need for therapies neutralizing JVC for prevention, management and treatment of JCV infection and PML Goldmann et al. demonstrated that neutralizing activity with JCV VP1 protein in sera of a rabbit (see Goldmann C. et al., 1999, J Virol.; 73(5): 4465-4469). Therapies based on neutralizing JCV antibodies could be applied for treatment, management and/or prevention of PML. Recently, immunological approaches have been under investigation and neutralizing antibodies binding to JC virus, especially targeting the VP1 protein, have been developed e.g. as described in US Patent Publication US2015/0191530, US2015/0056188 and US201510050271, the contents of each of which are incorporated herein by reference in their entirety. Such antibodies may cause reduction of JCV replication, proliferation or infectivity. Antibodies may bind to a conformational epitope of JCV VP1 protein or to the sialic acid binding pocket of VP1 protein of JCV.

In some embodiments, methods of the present disclosure may be used to prevent, manage and/or treat JCV infection and/or PML

AAV particles and methods of using the AAV particles described in the present disclosure may be used to prevent, manage and/or treat JCV. As a non-limiting example, the AAV particles of the present disclosure comprise a nucleic acid sequence encoding at least one of the sequences described in Table 3 (SEQ ID NO: 1740-2141).

### Influenza Virus

Influenza viruses cause a common respiratory infection called influenza (flu). Influenza viruses are categorized into three main groups, virus A, B and C. Influenza viruses are negative-sense, single-stranded, segmented RNA viruses. Influenza A contains two proteins on the surface of the viral envelope: hemagglutinin (H), which is a protein responsible for red blood cell agglutination and neuraminidase (N), which is an enzyme cleaving the glycosidic bonds of neuraminic acid. Influenza A mutates at a faster rate than types B and C. Several serotypes of H and subtypes of N have been identified. Influenza Type B, similarly to Type A, contains H and N protein. Type C influenza virus is a single stranded RNA virus with glycoprotein called hemagglutinin-esterase fusion. Influenza strains vary according to geographical presentation.

Influenza in general is a highly contagious disease and may be transmitted by the respiratory route. Influenza symptoms include e.g. high fever, runny nose, headache, sore throat, muscle pain, cough and occasionally nausea and vomiting. Influenza may lead to other complications such as pneumonia or sinus infections, Influenza may be dangerous to young children, the elderly, pregnant women and individuals with chronic medical conditions or weakened immune system. According to Centers for Disease Control and Prevention (CDC), the estimated annual number of flu-associated deaths in the United States ranges between 3000 and 49, 000, depending on the severity of the seasonal variations.

Influenza may be treated with good supportive care and antiviral medication. Antiviral medications include neuraminidase inhibitors, e.g. oseltamivir and zanamivir and M2 protein inhibitors. However, some strains of influenza appear to be resistant to these antiviral medications. Seasonal vaccinations to influenza are very efficient in prevention of the disease and are recommended annually.

There remains a need for prevention and treatment therapies for influenza, especially for those providing long lasting and broad neutralization. Therapeutic antibodies against influenza viruses have been developed. In general, antibody responses to different subtypes and serotypes of influenza A, B and C are unique. Some therapeutic antibodies are specific to an antibody type, whereas some have a broad coverage. Navivumab (developed by Celltrion, Inc.) taught in US Patent application US20140234336, firivumab (developed by Celltrion, Inc.) taught in US Patent application US20130004505 and diridavumab (developed by Jansen Biotech, Crucell and Johnson & Johnson) taught in International Patent application WO/2008/028946 are examples of therapeutically antibodies against influenza A hemagglutinin HA.

AAV particles and methods of using the AAV particles described in the present disclosure may be used to prevent, manage and/or treat influenza. As a non-limiting example, the AAV particles of the present disclosure comprise a nucleic acid sequence encoding at least one of the sequences described in Table 3 (SEQ ID NO: 1740-2141).

### Hepatitis

Hepatitis is an inflammation of the liver. Hepatitis may be caused by an infection of hepatitis viruses A, B, C, D or E. In some cases, hepatitis may be asymptotic. A typical symptom of hepatitis is jaundice, characterized by yellowing of the skin, mucous membrane and conjunctiva. Other symptoms include loss of appetite, diarrhea, nausea and fever. Hepatitis may lead to a liver failure. Acute form of hepatitis is healed within six months of infection. The inflammation may also progress to a chronic hepatitis, which may lead to liver complications such as fibrosis, cirrhosis or hepatocellular carcinoma. There is no specific treatment for hepatitis. Typically, acute hepatitis is treated with good supportive care, including good nutritional balance, fluid and rest. Chronic hepatitis may be treated with antiviral drugs. Hepatitis may be prevented by vaccinations.

Hepatitis A (HAV) virus belongs to the family of Picornaviridae. HAV is encapsidated in an icosahedral structure formed by 60 copies of three viral structural proteins (VP1, VP2 and VP3), (see e.g. Kim et al. 2004, Virology; 318(2):598-607, and references therein). HAV is spread by the fecal-oral-route. Typical transmission is through contaminated food or drink or in contact with an infected individual. Improperly cooked shellfish is a common source of HAV. Hepatitis A is more abundant in developing countries with poor sanitary conditions. According to the World Health Organization (WHO), an estimated 1.4 million people are infected by HAV every year.

Vaccines for prevention of HAV infection exists and are recommended to be administered to children under 1 year of age by CDC. As of today, there is no specific treatment for HAV infection. The treatment includes supportive therapy and may last for weeks or even months. There remains a need for treatment therapies for HAV. Antibodies for prevention and/or treatment of HAV have been developed. For example, US Patent US763476, International Publication WO2011114353 and Kim et al in Virology. 2004 Jan. 20; 318(2):598-607, the contents of each of which are incorporated herein by reference in their entirety, teach neutralizing antibodies targeting HAV antigens.

Hepatitis B (HBV) belongs to the family of Orthohepadnaviridae. HBV comprises a 3.2 kb-partially double-stranded circular DNA genome. HBV virus may be transmitted via the sexual transmission route, vertical transmission at birth, iatrogenic route (e.g. blood transfusions, contaminated reused needles and syringes), as well as via exposure to certain body fluids of an infected individual. According to the WHO, an estimated 240 million people are chronically infected with hepatitis B annually, and more than 780 000 people die to associated complications.

HBV may be prevented by vaccination. The WHO recommends vaccination for all infants, as well as for adults living in increased risk of the infection. HBV infection may be treated with antiviral medications, e.g. tenofovir and entecavir. The medication does not cure the disease but suppresses the replication of the virus. Individuals with chronic hepatitis B infection are administered antiviral medications for life. There remains a need for therapies providing long lasting management and/or cure for HBV infection. Antibodies for prevention and/or treatment of HBV infection are described e.g. in US Patent publication US20120308580 and International publication WO2013165972, the contents of each of which are herein incorporated by their reference in their entirety.

Hepatitis C (HCV) belongs to the family of Faviviridae. HCV is a positive-sense single-stranded RNA virus with an open reading frame with 9600 nucleotide bases. HCV is most commonly transmitted by the sexual transmission route or latrogenic route. Hepatitis C may be transmitted also via the vertical route, though uncommon. According to WHO, 130-150 million people have a chronic HCV infection and approximately half a million people die from complications associated with HCV annually.

As of today, there is no vaccine for HCV infection. Traditional treatment of hepatitis C is based on antiviral medication therapy with e.g. ribavirin and interferon. More recently, direct antiviral agents (DAA) have been developed to treat hepatitis C infections. However, there remains a need for efficient prevention and treatment therapies for HCV infection.

Hepatitis D (HDV) is a small spherical enveloped RNA virus belonging to the genus of deltaviruses. HDV infection may only replicate in the presence of a HBV virus and therefore HDV infection has a dependency on HBV. HDV virus may be transmitted as coinfection with HBV or be superimposed on chronic HBV or HBV carrier state. HDV may be transmitted similarly to HBV, e.g. via the sexual transmission route, vertical transmission at birth, latrogenic route, as well as via exposure to certain body fluids of an infected individual. Treatment and vaccination against HBV may be applied against HDV, and there remains a need for therapies to cure both infections.

Hepatitis E (HEV) is a linear, monoparte, single-stranded RNA virus belonging to the family of Hepevirdae. HEV may be transmitted via the fecal-oral route due to contaminated food or beverage, the iatrogenic route (e.g. blood transfusions, contaminated reused needles and syringes) or the vertical transmission route during pregnancy. Contaminated drinking water is the most common source of infection. Improperly cooked shellfish are a common source of HEV. The disease is present worldwide but is more abundant in East and South Asia, and especially in environments with poor sanitation and hygiene. According to WHO, an estimated 20 million HEV infections occur annually leading to 56 600 death associated with HEV complications.

There is no specific treatment for HEV. The disease is typically cured with good supportive care. As of today, vaccinations against HEV are not globally available, though development in the field has been done. There remains a need for prevention and treatment therapies for HEV infection. Antibodies for prevention and treatment of HEV have been developed. For example, neutralizing antibodies targeting HEV have been taught in US Patent U.S. Pat. No. 7,148,323, Tang et al. 2011, Proc. Natl. Acad. Sci. U.S.A. 108(25), 10266.10271 and Gu et al. 2015, Cell Res. 25(5), 604-620, the contents of each of which are incorporated herein by reference in their entirety.

In some embodiments, methods of the present disclosure may be used to prevent, manage and/or treat infections and complications caused by HAV, HBV, HCV, HDV and/or HEV.

AAV particles and methods of using the AAV particles described in the present disclosure may be used to prevent, manage and/or treat HAV. As a non-limiting example, the AAV particles of the present disclosure comprise a nucleic acid sequence encoding at least one of the sequences described in Table 3 (SEQ ID NO: 1740-2141).

AAV particles and methods of using the AAV particles described in the present disclosure may be used to prevent, manage and/or treat HBV. As a non-limiting example, the AAV particles of the present disclosure comprise a nucleic acid sequence encoding at least one of the sequences described in Table 3 (SEQ ID NO: 1740-2141).

AAV particles and methods of using the AAV particles described in the present disclosure may be used to prevent, manage and/or treat HDV. As a non-limiting example, the AAV particles of the present disclosure comprise a nucleic acid sequence encoding at least one of the sequences described in Table 3 (SEQ ID NO: 1740-2141).

AAV particles and methods of using the AAV particles described in the present disclosure may be used to prevent, manage and/or treat HEV. As a non-limiting example, the AAV particles of the present disclosure comprise a nucleic acid sequence encoding at least one of the sequences described in Table 3 (SEQ ID NO: 1740-2141).

### Respiratory Syncytial Virus (RSV)

Respiratory syncytial virus (RSV) is a single-stranded RNA virus belonging to the family of Paramyxoviridae. The RSV RNA is contained in a nucleocapsid made of 11 proteins and covered with a lipid envelope (see, e.g. Piedimonte, 2015, Cleve Clin J Med.; 82(11 Suppl 1):S13-8, and references therein). RSV attaches to the epithelial cells of the host airway cells with the surface glycoproteins G and F and merges the viral envelope to the membranes of adjacent cells. G and F glycoproteins are the principal antigens exposed to the host immune system.

Respiratory syncytial virus (RSV) causes infections of the respiratory tract including the lungs and breathing passages. RSV is transmitted through the respiratory transmission route, in direct contact with nasal or oral secretions of infected individuals, or indirectly e.g. by touching a contaminated surface. The symptoms include a runny nose, decrease of appetite, coughing, sneezing, fever and wheezing. The infection may progress into a pneumonia or bronchiolitis. Additionally, RSV infection may have a role in triggering asthma attacks and in the inception of asthma for individuals with a family history of asthma. In healthy adults, RSV infection is typically mild and does not require hospitalization. However, the infection may be dangerous for young children and infants, and for individuals with a weakened immune system. According to the CDC, almost all children under 3 years of age will acquire an RSV infection and up to 2% of cases require hospitalization. RSV infection the most common cause for bronchiolitis and pneumonia in children younger than 1-year-old.

As of today, there is no specific medical treatment for RSV infection on the market and typically the infection is treated with good supportive care. There remains a need for prevention and treatment therapies for RSV infections and associated complications. Antibodies for treatment and prevention of RSV infection have been developed. For example, palivizumab (developed by MedImmune) taught in US Patent U.S. Pat. No. 8,153,133, the contents of which are incorporated herein by reference in their entirety, is a nearly human monoclonal antibody targeting the RSV F glycoprotein. Palivizumab is used for passive immunity for infants at risk for severe infection, including children with hemodynamically significant congenital heart defects, profound immunodeficiency and pulmonary or neuromuscular pathologies impairing airway clearance.

In some embodiments, methods of the present disclosure may be used to prevent, manage and/or treat infections and complications caused by RSV.

AAV particles and methods of using the AAV particles described in the present disclosure may be used to prevent, manage and/or treat RSV. As a non-limiting example, the AAV particles of the present disclosure comprise a nucleic acid sequence encoding at least one of the sequences described in Table 3 (SEQ ID NO: 1740-2141).

### Herpes Simplex Virus 1 and 2

Herpes simplex viruses 1 and 2 (HSV1 and HSV2), also known as human herpesvirus 1 and 2 (HHV-1 and HHV-2), belong to the family of Herpesviridae. Herpesviruses in general, consist of an icosahedral capsid surrounded by a membrane envelope. The capsid contains the viral double stranded DNA. The capsid is surrounded by an amorphous tegument of 30 viral proteins. The virion is enveloped by lipids with multiple viral glycoproteins and cellular proteins (see, e.g. McAllister and Schleiss, 2014, Expert Rev Vaccines; 13(11): 1349-1360, and references therein).

HSV1 and HSV2 cause an infection known as herpes, which is characterized by blisters in the skin, or mucous membranes of the mouth, lips, also known as “cold sores”, or genitals. Typically, the symptoms are mild or asymptomatic. However, HSV1 and HSV2 are neurotropic and neuroinvasive viruses persisting in the body by becoming latent, and sustain in the cell bodies of neurons. The infection is lifelong with outbreaks, or sporadic episodes of viral reactivation, when the virus in the nerve cells become active causing new blistering. The infection may be dangerous to individuals with weakened immune system. Neonatal herpes of infants may be fatal. Occasionally HSV1 infections may lead to encephalitis or keratitis. HSV1 and HSV2 are transmitted by contact with an infected area during reactivations of the virus. HSV1 is mainly transmitted by oral-to-oral contact, skin contact or the sexual transmission route. HSV1 may also be transmitted vertically during birth. HSV2 is transmitted via the sexual transmission route and is one of the most common sexually transmitted infections. According to the WHO, an estimated 67% of world's population aged under 50 years has an HSV-1 infection. An estimated 11% of world's population aged 15-49 years has an HSV2 infection.

As of today, there is no vaccination for prevention of HSV1 and HSV2 infections on the market. HSV1 and HSV2 infections may be treated with antiviral medications, such as acyclovir, famciclovir and valacyclovir. Antiviral medications do not cure the infection, but reduce the severity and frequency of symptoms. There remains a therapy for prevention and cure for these infections. Antibodies for prevention, treatment and management of HSV1 and HSV2, targeting the viral glycoproteins, have been developed, as described e.g. in US Patent U.S. Pat. No. 8,431,118, US Patent U.S. Pat. No. 5,646,041, Haynes US Patent Publication US2014/0302062, the contents of each of which are incorporated herein by reference in their entirety.

In some embodiments, methods of the present disclosure may be used to prevent, manage and/or treat infections and complications caused by HSV1 and HSV2.

AAV particles and methods of using the AAV particles described in the present disclosure may be used to prevent, manage and/or treat HSV. As a non-limiting example, the AAV particles of the present disclosure comprise a nucleic acid sequence encoding at least one of the sequences described in Table 3 (SEQ ID NO:1740-2141).

### Human Cytomegalovirus

Human Cytomegalovirus (HCMV) also known as human herpesvirus 5 (HHV-5) belongs to the family of Herpesviridae, a sub-family of Betaherpesvirinae. HCMV is a double-stranded DNA enveloped virus composed of a nucleocapsid surrounded by structured tegument layer and bounded by a trilaminate membrane envelope.

In most occasions, an initial HCMV infection is asymptomatic, or associated with mild symptoms e.g. sore throat, fatigue, flu-like symptoms, and fever. After initial infections, HCMV virus resides in mononuclear cells without detectable symptoms. HCMV infection may be dangerous to individuals with weakened immune system. HCMV may be transmitted by contact with certain body fluids of an infected individuals (e.g. saliva, urine, semen). HCMV may be transmitted vertically, especially if acquired during pregnancy, leading to a congenital HCMV infection. According to CDC, about 1 in 150 children are born with congenital CMV infection. In about 20% of cases, congenital HCMV infection may lead to premature birth, birth defects or developmental disabilities, e.g. liver, lung, spleen problems, small head size, small body size or seizures.

As of today, there is no specific treatment or prevention therapy for HCMV infection. In severe cases of congenital HCMV infection, infants may be treated with an antiviral drug, ganciclovir, to prevent hearing loss and developmental outcomes. However, the drug has serious side effects. There remains a need for prevention therapy and improved therapies for treatment and cure of HCMV infection. Antibodies neutralizing HCMV have been developed. Such antibodies are taught e.g. in International Patent Publication WO2010007463, US Patent U.S. Pat. Nos. 9,149,524, 8,492,529 and 8,202,518, the contents of each of which are incorporated herein by reference in their entirety.

In some embodiments, methods of the present disclosure may be used to prevent, manage and/or treat infections and complications caused by HCMV.

AAV particles and methods of using the AAV particles described in the present disclosure may be used to prevent, manage and/or treat HCMV. As a non-limiting example, the AAV particles of the present disclosure comprise a nucleic acid sequence encoding at least one of the sequences described in Table 3 (SEQ ID NO: 1740-2141).

### Epstein-Barr Virus

Epstein-Barr virus (EBV), also known as human herpesvirus 4 (HHV-4) belongs to the family of Herpesviridae. EBV is a double-stranded DNA virus composed of a protein nucleocapsid surrounded by a tegument layer and bounded by an envelope containing lipids and surface projection of glycoproteins. EBV may enter B cells and epithelial cells.

EBV infection causes glandular fever known as infectious mononucleosis, also known as the kissing disease. Typical symptoms include e.g. sore throat, fever swollen lymph nodes in the neck, enlarged spleen, swollen liver, rash and fatigue. Additionally, EBV infection is associated with certain cancers, e.g. central nervous system lymphomas, Hodgkin's lymphoma, Burkitt's lymphoma, Guillain-Barre syndrome, multiple sclerosis, and higher susceptibility to certain autoimmune diseases. The virus is transmitted via contact with certain bodily fluids of an infected individual, especially through saliva. The infection affects majority of population. According to CDC, 90% of adult population have antibodies demonstrating current or past EBV infection.

As of today, there is no specific therapy for prevention or treatment of EBV infection on the market. Typically, EBV infection is treated with good supportive care. Antibodies for prevention, management and treatment of EBV infection and associated diseases have been developed, e.g. by Wang and Fogg in US Patent publication US20150064174 and Fang et al. in Intervirology 50 (4), 254.263 (2007), the contents of each of which are incorporated herein by reference in their entirety.

In some embodiments, methods of the present disclosure may be used to prevent, manage and/or treat infections and complications caused by EBV.

AAV particles and methods of using the AAV particles described in the present disclosure may be used to prevent, manage and/or treat EBV. As a non-limiting example, the AAV particles of the present disclosure comprise a nucleic acid sequence encoding at least one of the sequences described in Table 3 (SEQ ID NO: 1740-2141).

### Varicella Zoster Virus

Varicella zoster virus (VZV), also known as human herpes virus 3 (HHV-3) and chickenpox virus, belongs to the family of Herpesviridae. VZV is a linear duplex DNA molecule containing two segments (L and S) joined covalently. At least five clades of the virus have been identified.

VZV causes varicella, also known as chickenpox, which is an infection characterized by blister-like rash, itching, fatigue and fever. Chickenpox may be dangerous for babies, adults and individuals with weakened immune system. After primary phase of the infection, VZV resides in the nerves, including cranial nerve ganglia, dorsal root ganglia and autonomic ganglia, and may eventually lead to shingles, which is a viral disease characterized with a painful skin rash, blistering and occasionally nerve pain. Additionally, VZV has been associated with other complications, e.g. neurological conditions, inflammation of arteries, myelitis, Ramsay Hunt syndrome, Mollaret's meningitis. VZV is transmitted by direct contact or by the respiratory route. VZV is highly contagious. According to CDC, before VZV vaccination, about 4 million people would be affected by chickenpox in the US annually, with more than 10, 000 hospitalized.

VZV infection may be prevented by a vaccination, which is recommended by CDC to all children and unvaccinated adults. Chickenpox may be treated with antiviral medications, e.g. acyclovir, valacyclovir and famciclovir, or with other symptom relieving medications and therapies. However, the present antiviral medications may have undesirable side effects. There remains a need for improved therapies to treat VZV infection, and its reactivation stages. Antibodies targeting VZV have been developed, e.g. as described in US Patent U.S. Pat. No. 5,506,132, and US Patent application US20100074906, the contents of which are herein incorporated by their reference in their entirety.

In some embodiments, methods of the present disclosure may be used to prevent, manage and/or treat infections and complications caused by VZV.

AAV particles and methods of using the AAV particles described in the present disclosure may be used to prevent, manage and/or treat VZV. As a non-limiting example, the AAV particles of the present disclosure comprise a nucleic acid sequence encoding at least one of the sequences described in Table 3 (SEQ ID NO: 1740-2141).

### Coronaviruses

Coronaviruses are a diverse group of enveloped viruses belonging to the family of Coronaviridae. Coronaviruses contain an envelope, a helical capsid, and a single-stranded, positive-sense RNA genome. Coronaviruses have a characteristic structure with viral spike-shaped glycoprotein populating the surface of the virus and causing an appearance resembling the solar corona. Coronaviruses are a common cause of mammalian and avian infections causing upper respiratory tract, gastrointestinal and central nervous system diseases.

Human coronavirus 229E, OC-43, NL63, and HKU1 are a cause a behind typical, short term ‘common cold’ and affect individuals all over the world. Typical symptoms of the infections include coughing, sneezing, fatigue and fever. Occasionally the viruses can cause lower-respiratory tract illnesses, such as pneumonia. The viruses are spread by direct contact or by the respiratory route. The infections may be dangerous to the elderly and individuals with weakened immune system. There is no specific treatment or prevention therapy for these coronavirus infections.

Severe Acute Respiratory Syndrome coronavirus (SARS-CoV) causes a viral respiratory illness. Typical symptoms of the infection include a high fever, headache, body aches, dry coughing and eventually pneumonia. SARS-CoV was identified in 2003 in an outbreak starting from Asia. SARS-CoV is transmitted by direct contact with an infected individual or by the respiratory route. According to the WHO, during the 2003 outbreak of SARS-CoV, 8098 people worldwide were infected with symptoms and out of them, 774 died. As of today, there is no specific treatment or prevention therapy for SARS on the market. Antiviral medication and steroids may be prescribed to certain patients. Antibodies targeting SARS-CoV have been developed, e.g. as described in US Patent U.S. Pat. No. 7,728,110 and US Patent publication US20110159001, the contents of each of which are herein incorporated by their reference in their entirety.

Middle East Respiratory syndrome coronavirus (MERS-CoV) causes an acute severe respiratory infection affecting the lungs and breathing tubes. MERS-CoV was identified in 2012. Typical symptoms include fever, cough and shortness of breath, eventually pneumonia and additionally gastrointestinal symptoms. MERS-CoV is highly dangerous to humans. According to the WHO, 36% of the infections are fatal. MERS-CoV is a zoonotic virus transmitted to humans from animals, e.g. bats and camels, or from human to human. Camels are suggested to be a reservoir for MERS-CoV. Majority of MERS-CoV infection have occurred in the Arabian Peninsula, and especially in Saudi Arabia. As of today, no specific treatment of prevention therapy for MERS-CoV infection is available on the market. Antibodies targeting MERS-CoV have been developed, e.g as described in International publication WO201505742, the contents of which are herein incorporated by their reference in their entirety.

In some embodiments, methods of the present disclosure may be used to prevent, manage and/or treat infections and complications caused by SARS-CoV, MERS-CoV and/or other coronaviruses.

AAV particles and methods of using the AAV particles described in the present disclosure may be used to prevent, manage and/or treat coronaviruses. As a non-limiting example, the AAV particles of the present disclosure comprise a nucleic acid sequence encoding at least one of the sequences described in Table 3 (SEQ ID NO: 1740-2141).

### Poxviruses

Poxviruses affecting humans include orthopoxvirus, parapoxvirus, yatapoxvirus and mollusipoxvirus. Poxviruses are typically brick-shaped, enveloped, single, liner or double-stranded viruses with DNA genome. Typically, poxvirus infections cause lesions, skin nodules, or disseminated rash. Poxviruses may be transmitted by direct contact with contaminated humans, animals or materials. Diseases caused by poxviruses include e.g. smallpox, monkeypox, molluscum conagiosum, vaccinia virus and orf virus infection.

Smallpox virus infection is highly fatal, and though it does not occur in nature anymore, smallpox virus is considered to be a potential chemical or biological warfare agent. The threat of terrorism has created a need for efficient and improved methods for treatment and/or prevention of smallpox infection. The traditional vaccination for smallpox, also applicable against monkeypox, has a rare but severe side effect due to vaccinia virus, which is the active constituent of the vaccine that eradicated smallpox. Vaccinia Immune Globulin (VIG) is the only licensed therapeutic treatment for smallpox, but is highly variable and available in limited quantities. Antibodies against smallpox have been developed, as described e.g. in US Patent U.S. Pat. No. 8,623,370 and US Patent publication US20140186370, the contents of each of which are herein incorporated by their reference in their entirety.

In some embodiments, methods of the present disclosure may be used to prevent, manage and/or treat infections and complications caused by smallpox virus and/or other poxviruses.

AAV particles and methods of using the AAV particles described in the present disclosure may be used to prevent, manage and/or treat poxvirus. As a non-limiting example, the AAV particles of the present disclosure comprise a nucleic acid sequence encoding at least one of the sequences described in Table 3 (SEQ ID NO: 1740-2141).

### Enterovirus 71

Enterovirus 71 (EV71) belongs to the family of Picornaviridae. Enterovirus 71 is a single-stranded RNA positive sense virus. The virus has approximately 7411 nucleotides. The RNA genome is enclosed in an icosahedral capsid of structural proteins VP1-VP4. (see, e.g. Tan et al., 2014, J Biomed Sci; 21(1): 140, and references therein).

EV71 infections typically cause hand, foot and mouth (HFMD), which is characterized by fever, mouth ulcers, and vesicles on the palms of the hands and feet. Additionally, EV71 causes severe neurological manifestations, including poliomyelitis-like acute flaccid paralysis, brainstem encephalitis in infants and children. These neurological manifestations may be fatal, or cause permanent neurological consequences, such as delayed neurodevelopment or reduced cognitive function in children. EV71 is transmitted through direct contact with certain bodily fluids, such as saliva, or the respiratory route, or the fecal-to-mouth route. Outbreaks of EV71 have been reported by WHO in the US, Europe, and more frequently in Asia-Pacific region in the past 30 year.

As of today, no specific treatment or prevention therapy for EV71 is on the market. Antiviral drugs, e.g. pleconaris and other capsid-function inhibitors (see, e.g. Tan et al. J Biomed Sci. 2014; 21(1): 140), may be prescribed against EV71 infections, though their effectiveness is not well established. There remains a need for prevention and treatment therapies for EV71 infection. Antibodies neutralizing EV71 have been developed. Non-limiting examples include the anti-EV71 antibody MAB979 (developed by Merck Millipore) and those taught by Carderosa et al. in International Patent Publication WO2015092668, the contents of which are incorporated herein by reference in their entirety.

In some embodiments, methods of the present disclosure may be used to prevent, manage and/or treat infections and complications caused by EV71.

AAV particles and methods of using the AAV particles described in the present disclosure may be used to prevent, manage and/or treat EV71. As a non-limiting example, the AAV particles of the present disclosure comprise a nucleic acid sequence encoding at least one of the sequences described in Table 3 (SEQ ID NO: 1740-2141).

### Rubella Virus

Rubella virus belongs to the family of Togaviridae. Rubella virus is a positive sense, single-stranded RNA virus with spike-like, hemagglutinin containing surface projections. The virus core is enveloped by glycosylated E1 and E2 proteins.

Rubella, also known as German measles or three-day measles, is a viral infection typically characterized by a rash, low fever, nausea, swollen lymph glands behind the ears and the neck, and mild conjunctivitis. At later stage, the infection may develop arthritis and pain in the joints. Typical symptoms of rubella infection are mild and affect children and young adults. Rubella virus is transmitted by the respiratory route and the virus replicates in the nasopharyngeal mucosa and local lymph nodes. However, when an infection is acquired during pregnancy, the virus is transmitted through vertical route with 90% chance and may cause fetal death or congenital defects known as congenital rubella syndrome (CRS). Infants with CRS may have hearing impairments, eye and heart defects, diabetes mellitus, thyroid dysfunction and/or autism. According to the WHO, about 10,000 infants with CRS are born every year, majority occurring in countries with low vaccine coverage.

As of today, there is no specific treatment for rubella. Rubella may be prevented with vaccination, and rubella has been part of the vaccination program for the past 40 years. However, the infection still persists and an increasing concern related to the life-time of vaccine efficiency exists. There remains a need for long lasting prevention therapy, as well as treatment for rubella virus infection. Antibodies against rubella have been described e.g. in US Patent US20100143376, the contents of each of which are herein incorporated by reference in their entirety.

In some embodiments, methods of the present disclosure may be used to prevent, manage and/or treat infections and complications caused by rubella.

AAV particles and methods of using the AAV particles described in the present disclosure may be used to prevent, manage and/or treat Rubella. As a non-limiting example, the AAV particles of the present disclosure comprise a nucleic acid sequence encoding at least one of the sequences described in Table 3 (SEQ ID NO: 1740-2141).

### Human Papilloma Virus

Human papilloma virus (HPV) is a non-enveloped double-stranded DNA virus belonging to the family of Papillomaviridae. Over 170 types of HPV have been identified.

HPV infections may be asymptomatic, or cause infection related to warts (e.g. plantar, flat or anogenital warts), oral infections such as papillomas or multifocal epithelial hyperplasia. The infection may be undetected, and clears from the body to low levels within two years. Infections caused by human papillomavirus (HPV) have been associated with certain cancers of stratified epithelial tissues, e.g. cervical, anal, vaginal, vulvar and penile cancers, lung and throat cancers. Especially HPV16 and HPV18 are known to be carcinogenic. According to the WHO, persistent genital HPV infection may cause cervical cancer which is the second most common cancer in women worldwide. In developing countries, cervical cancer counts for 13% of all female cancers, and survivor rate worldwide is approximately 50%. HPV is very common. CDC estimates that every one in four individuals in the US has an HPV infection. Most commonly HPV is transmitted by the sexual route, but also the vertical transmission route, or by direct contact to infected blood, or objects may occur.

Cancers caused by HPV may be prevented by vaccines developed against certain HPV types. The vaccines are available worldwide and are recommended by CDC for all preteen aged children. As of today, there are no specific treatment for HPV infection. There remains a need for prevention and treatment therapy affecting a broad range of HPV infections. Antibodies for HPV have been developed, e.g. as described in International publication WO2015096269, the contents of each of which are herein incorporated by reference in their entirety.

In some embodiments, methods of the present disclosure may be used to prevent, manage and/or treat infections and complications caused by HPV.

AAV particles and methods of using the AAV particles described in the present disclosure may be used to prevent, manage and/or treat HPV. As a non-limiting example, the AAV particles of the present disclosure comprise a nucleic acid sequence encoding at least one of the sequences described in Table 3 (SEQ ID NO: 1740-2141).

### Pseudomonas Aeruginosa

Pseudomonas Aeruginosa (P. Aeruginosa) is a common Gram-negative, aerobic, rod-shaped bacterium belonging to the family of Pseudomonodaceae. P. Aeruginosa is found in soil, water, skin, flora, and in most man-made environments around the world. P. Aeruginosa is considered as an opportunistic pathogen taking advantage of a weakened immune system.

P. Aeruginosa may cause a variety of mild infections, such as, urinary tract infections, respiratory system infections, dermatitis, soft tissue infections, bacteremia, bone and joint infections, gastrointestinal infections, blood infections, ear infections, skin rash, eye infections and a variety of systemic infections. P. Aeruginosa is transmitted through water, contaminated hands, materials or objects. In general, P. Aeruginosa infections in healthy individuals are very mild or asymptomatic. However, the infections expose a significant risk for individuals with weakened immunity, such as patients with other underlying illnesses or complications, and especially when in a hospital environment. For example, patients with cystic fibrosis have a susceptibility towards loss of lung function due to respiratory tract infection with the bacterium. Patients attached to breathing machines, patients with catheters, or with surgery wounds or burn wounds are potentially at risk for serious and life-threatening infections. P. Aeruginosa infection may lead to a fatal sepsis. According to CDC, approximately 51, 000 health-care associated infection occur in the US every year, leading to approximately 400 deaths.

As of today, there are no prevention therapies for P. Aeruginosa infection on the market. Some strains of P. Aeruginosa may be treated with antibiotics, e.g. gentamicin, tobramycin, colistin, and amikacin. However, an increasing number of strains of P. Aeruginosa, especially those affecting hospitalized patients, are resistant to antibiotics and no specific treatment therapy exists. There remains a need for improved treatment and prevention therapies against P. Aeruginosa infections. Antibodies against P. Aeruginosa have been developed, such as, panobacumab (developed by Kenta Biotech Inc.), which is an antibacterial antibody against P. Aeruginosa.

In some embodiments, methods of the present disclosure may be used to prevent, manage and/or treat infections and complications caused by P. Aeruginosa.

AAV particles and methods of using the AAV particles described in the present disclosure may be used to prevent, manage and/or treat P. Aeruginosa. As a non-limiting example, the AAV particles of the present disclosure comprise a nucleic acid sequence encoding at least one of the sequences described in Table 3 (SEQ ID NO: 1740-2141).

### Streptococcus Bacteria

Streptococcus is a genus of gram-positive bacteria belonging to the family of Streptococcaceae. Species of Streptococcus are divided into alpha- and beta-hemolytic species. Alpha-hemolytic species cause oxidation of iron in hemoglobin molecules within the red blood cells. Alpha-hemolytic streptococci include e.g. Streptococcus pneumoniae and Streptococcus viridans. Beta-hemolytic species cause complete rupture of the red blood cells and include e.g. Lancefield groups A and B, also known as ‘group A strep’ and ‘group B strep’. Streptococcus genus includes overall more than 50 species. Streptococcus bacteria cause a variety of infections in humans, including dental caries, pneumonia, endocarditis, meningitis, respiratory tract infections, urinary tract infections, neonatal meningitis, pharyngitis and/or sepsis.

Streptococcus pneumoniae is a common bacterium causing, i.e. pneumonia, meningitis, bronchitis, acute sinusitis, conjunctivitis, osteomyelitis, endocarditis and/or septic arthritis. The bacteria is transmitted by direct contact or via the respiratory route. The bacteria resides in the nasopharynx of healthy carriers and proceeds into an infection under certain circumstances. The infection may be prevented by vaccines, e.g. conjugate vaccine or polysaccharide vaccines. The infection may be treated with antibiotics, e.g. broad-spectrum cephalosporin, and vancomycin, but there is a concern over increasing resistant towards antibodies. According to CDC, Streptococcus pneumoniae is currently resistant to one or more antibiotics in 30% of infections. Streptococcus pneumoniae is resistant to e.g. penicillins. There remains a need for improved, non-antibiotic, therapies for treatment of Streptococcus pneumoniae and other Streptococcus infections. Antibodies for Streptococcus have been developed, as described e.g. in US Patent U.S. Pat. No. 5,686,070 and US Patent publication US20070003561, the contents of each of which are herein incorporated by reference in their entirety.

In some embodiments, methods of the present disclosure may be used to prevent, manage and/or treat infections and complications caused by Streptococcus pneumoniae and other Streptococcus bacteria.

AAV particles and methods of using the AAV particles described in the present disclosure may be used to prevent, manage and/or treat Streptococcus pneumoniae. As a non-limiting example, the AAV particles of the present disclosure comprise a nucleic acid sequence encoding at least one of the sequences described in Table 3 (SEQ ID NO: 1740-2141).

Staphylococcus bacteria

Staphylococcus is a genus of gram-positive bacteria belonging to the family of Staphylococcaceae. The genus includes overall approximately 40 species. Most species of the genus are harmless and reside in the skin and mucous membranes of humans. Staphylococcus bacteria may also be found in the soil. The bacteria may cause diseases either through toxin production or penetration. Staphylococcal toxins are a common cause of food poisoning. Staphylococcus bacteria may cause a variety of diseases, e.g. localized or diffuse skin infection, gastroenteritis, ear infections, septic arthritis, osteomyelitis, sinusitis, infective endocarditis and/or toxic shock syndrome.

Staphylococcus aureus (S. aureus) is typically residing in human nose asymptomatically. In certain circumstances, S. aureus infections may affect many tissues and organs. Individuals with chronic conditions, e.g. diabetes, cancer, vascular disease, eczema and lung disease, have an increased susceptibility towards S. aureus infections. S. aureus may cause skin infections, such as, pimples, impetigo, atopic dermatitis, cellulitis folliculitis. More serious forms of infections include pneumonia, meningitis, osteomyelitis and endocarditis. S. aureus may also cause food poisoning. In severe cases, S. aureus infection may enter the blood stream causing bacteremia and/or sepsis. As of today, there is no medical therapy for prevention of the infection. Some strains of S. aureus may be treated with antibiotics. However, increasing resistance towards antibiotics is a concern. Currently several antibiotic resistant forms of S. aureus exist, including, but not limited to, Methicillin-resistant Staphylococcus aureus (MRSA), Vancomycin-intermediate Staphylococcus aureus (VISA) and Vancomycin-resistant Staphylococcus aureus (VRSA). The drug resistant forms of S. aureus are more frequent in hospital environments.

Staphylococcus epidermidis (S. epidermidis) resides in the normal human skin flora and may cause an infection to individuals with weakened immune system, and to individuals who have catheters, prostheses or surgical implants. S. epidermidis has an ability to colonize on plastic materials or devices placed within the body. The infection may be treated with some antibiotics, but they do not remove the infection and can only be used to manage such infections. Many S. epidermis strains are resistant to antibiotics, such as penicillin, methicillin and/or amoxicillin, and increasing resistance to antibiotics in a concern.

There remains a need for prevention and/or improved treatment therapies against Staphylococcal infections. Antibodies targeting Staphylococcal bacteria have been developed. As an example, pagabaximab (developed by MedImmune and AstraZeneca) is a monoclonal antibody for prevention of staphylococcal sepsis and may be administered to infants with low birth weight.

In some embodiments, methods of the present disclosure may be used to prevent, manage and/or treat infections and complications caused by Staphylococcus bacteria.

AAV particles and methods of using the AAV particles described in the present disclosure may be used to prevent, manage and/or treat Staphylococcal infections. As a non-limiting example, the AAV particles of the present disclosure comprise a nucleic acid sequence encoding at least one of the sequences described in Table 3 (SEQ ID NO: 1740-2141).

Clostridium tetani

Clostridium tetani(C. tetani) is a rod-shaped, anaerobic, Gram-positive bacteria belonging to the family of Clostridiaceae. A matured bacterium develops a terminal spore, which is resistant to heat and common antiseptics. C. tetani produces tetanospasmin toxin. C. tetani is found as spores in soil and in the gastrointestinal tract of animals.

C. tetani infection spreads the tetanospamin toxin to the body, causing tetanus, also known as lock jaw. Tetanus is a dangerous disease characterized by painful tightening of the muscles. The disease may lead to locking of the jaw and neck, leading to inability to open mouth or swallow. The tightening may affect the whole body. In severe cases, the infection may lead to breathing difficulties, pneumonia, or pulmonary embolism. Even more serious is an infection acquired during pregnancy, leading to almost always fatal neonatal tetanus of an infant. The bacteria is typically transmitted through broken skin by direct contact with contaminated soil or objects, or saliva or feces of a contaminated animal. Especially susceptible are individuals with burns, puncture wounds, crush injuries or injuries with dead tissue, individuals having animal bites or scratches. Tetanus is fairly uncommon in developed countries. However, the WHO reported an estimated 50, 000 neonatal tetanus deaths in year 2008. A program form elimination of tetanus was started in 1989 by the WHO.

Tetanus may be prevented efficiently by a four vaccine combination, DTaP, Tdap, DT, and Td, given to children and adults. For adequate immunity, the primary vaccine is administered during childhood, a booster dose during adolescence and every 10 years thereafter during adulthood. C. tetani infection may be treated with antibiotics, wound care and with human tetanus immune globulin (an antitoxin). Despite the existing treatment methods, approximately 10% of tetanus infections lead to death, according to CDC. There remains a need for longer lasting vaccine as well as improved treatment therapies against C. tetani infections. Antibodies against C. Tetani have been developed, as described e.g. by Larrick, J. W. et al., 1992, Immunol. Rev. 130, 69-85, and de Kruif, J. et al., 2009, J. Mol. Biol. 387 (3), 548-558, the contents of each of which are herein incorporated by reference in their entirety.

In some embodiments, methods of the present disclosure may be used to prevent, manage and/or treat infections and complications caused by C. Tetani.

MV particles and methods of using the AAV particles described in the present disclosure may be used to prevent, manage and/or treat C. Tetani. As a non-limiting example, the AAV particles of the present disclosure comprise a nucleic acid sequence encoding at least one of the sequences described in Table 3 (SEQ ID NO: 1740-2141).

### Bordetella

Bordetella is a genus of Gram-negative, coccobacilli belonging to the family of Alcaliigenaceae. The structure of the bacteria consists of an outer membrane with lipopolysaccharides and phospholipids forming a capsule. Bordetella bacteria affecting humans include, but are not limited to, B. pertussis, B. parapertussis and B. bronchiseptica. B. pertussis resides in the upper air pathways, mostly the trachea and the bronchi, of humans. B. parapertussis resides in the upper air pathways of mammals. The bacteria release toxins that cause damage and swelling of the respiratory pathways.

Pertussis, also known as whooping cough, is a highly contagious infection of the respiratory track caused most commonly by B. pertussis, and occasionally by B. parapertussis. Typical symptoms of the infection include severe coughing and difficulty to breathe accompanied by a runny nose, apnea and fever. Additional complications for infants include pneumonia, convulsions, apnea, and encephalopathy. The bacteria are transmitted through the respiratory tract route. The disease is especially dangerous for infants. According to CDC, about 30,000 infections were reported in the US in 2014. CDC reports 277 deaths occurring from 2000 through 2014, out of which 241 where infants less than 3 months of age.

Pertussis may be treated with antibiotics, such as, erythromycin, clarithromycin or azithromycin. However, an increasing resistance to antibiotics is a concern. Pertussis caused B. pertussis may be prevented by vaccination, e.g. by DTaP combination vaccine, which is recommended routinely for infants by CDC and WHO. Despite the widespread vaccination, the disease has insisted. The protection provided by the traditional vaccination is estimated to be 3-6 years. There remains a need for prevention therapies providing a longer lasting immunity, as well as for improved, non-antibiotic, treatments. Antibodies for prevention and/for treatment of pertussis have been developed, as described e.g. in International publication WO2014160098, and Hussein, A. H. et al., 2007, Infect. Immun. 75 (11), 5476-5482, the contents of each of which are herein incorporated by reference in their entirety.

In some embodiments, methods of the present disclosure may be used to prevent, manage and/or treat infections and complications caused by B. pertussis, B. parapertussis and/or other Bordetella bacteria.

AAV particles and methods of using the AAV particles described in the present disclosure may be used to prevent, manage and/or treat Bordetella infection. As a non-limiting example, the AAV particles of the present disclosure comprise a nucleic acid sequence encoding at least one of the sequences described in Table 3 (SEQ ID NO: 1740-2141).

### Mycobacterium

Mycobacterium is a genus of nonmotile and aerobic bacteria, belonging to its own family of Mycobacteriaceae. Mycobacteria have an outer membrane, and a hydrophobic and waxy cell wall with mycolic acid/mycolates. The cell wall is neither truly Gram-positive nor -negative. In general, the infections are difficult to treat and the bacteria is naturally resistant to many antibiotics, e.g. penicillin, due to the cell wall. Mycobacteria includes species, such as, but not limited to, M. tuberculosis, Nontuberculous mycobacteria (NTM), M. leprae, M. bovis, M. africanum, and M. microti.

M. tuberculosis is a genetically diverse bacterium and most common and dangerous of the mycobacteria family species. M. tuberculosis causes tuberculosis (TB) which is an infection mainly affecting the lungs. Typical early symptoms include cough, fever, night sweat, and weight loss. The disease may be mild for a period of time and therefore early diagnosis is difficult. Eventually the symptoms get more severe and coughing sputum and blood may occur. TB may be transmitted by the respiratory tract. TB affects all ages of the population, but is most dangerous to children, and individuals with weakened immune systems, e.g. HIV patients. According to the WHO, TB is referred to as a top infectious disease killer worldwide. WHO reports an estimated 9.6 million infections of TB in 2014, out of which 1.5 million cases were fatal. The disease is globally spread, but it is most abundant in the South-East Asia and Western Pacific Regions.

TB may be prevented by vaccinations, i.e. Bacille Clamette-Guerin vaccine. The vaccine is provided for children and adults exposed to environments with high risk of infection. However, the vaccine is not always efficient against TB, e.g. due to the diversity of strains geographically. TB may be treated with a 6 to 9 month course of combinational antimicrobial drug therapy. Antimicrobial drugs effective against TB include e.g. isoniazid, rifampin, ethambutol, and pyrazinamide. However, an increasing resistance towards the medication is a concern. Certain strains of existing TB are identified as multi-drug resistant TB strains, which do not respond to therapy with e.g. isoniazid, rifampicin, or other common drugs. WHO reports an estimated 480 000 multidrug-resistant TB infections in 2014. There remains a need for prevention therapies protecting against broad spectrum of strains, as well as for improved treatment of M. tuberculosis and/or other mycobacteria. Antibodies against mycobacteria have been developed, as described e.g. in US Patent publications US20130309237, US20130309237, US20060229438, the contents of each of which are herein incorporated by reference in their entirety.

In some embodiments, methods of the present disclosure may be used to prevent, manage and/or treat infections and complications caused by M. tuberculosis and/or other mycobacteria.

AAV particles and methods of using the AAV particles described in the present disclosure may be used to prevent, manage and/or treat myobacterium related diseases. As a non-limiting example, the AAV particles of the present disclosure comprise a nucleic acid sequence encoding at least one of the sequences described in Table 3 (SEQ ID NO: 1740-2141).

### Francisella Tularensis

Francisella Tularensis (F. tularensis) is a facultative intracellular Gram-negative, rod-shaped bacterium belonging to the family of Francisellaceae. F. tularensis resides in invertebrates, birds, reptiles, fish, and mammals, including humans. It is one of the most infectious and pathogenic bacteria known (see, e.g. Pechous et al., 2009, Microbiol Mol Biol Rev.; 73(4): 684-711).

F. Tularensis causes infection called Tularemia. Severity of tularemia varies from mild to fatal. F. Tularensis may be transmitted to a human by direct skin or eye contact, by the respiratory route or by consumption of contaminated food or drink. Most commonly, the infection is acquired while handling infected animals. Most common form of tularemia is ulcero-glandular tularemia, characterized by skin ulcers on the site of infection accompanied by swelling or regional lymph glands. Ulcero-glandular tularemia is typically acquired by a tick, or deer fly bite. Pneumonic tularemia is an infection of the respiratory tract characterized by a cough, chest pain, and difficulty of breathing. Pneumonic tularemia is transmitted through the respiratory route and may be fatal if not treated. Oropharyngeal tularemia is transmitted by contaminated food or beverage and causes a sore throat, mouth ulcers, tonsillitis and swelling of lymph glands in the neck. Other forms of tularemia include glandular, oculo-glandular (affecting the eyes) and typhoidal (combination of the general symptoms). F. Tularensis is considered to be a potential biological and chemical warfare agent.

As of today, there is not preventive therapy for tularemia infection on the market. Some vaccines have been under development (see, e.g. Pechous et al., Microbiol Mol Biol Rev. 2009 December; 73(4): 684-711). Tularemia may be treated with antibiotics, such as, streptomycin, gentamicin, doxycycline, and ciprofloxacin. However, increasing resistance against antibiotics is a concern. There remains a need for improved prevention and treatment therapies for F. Tularensis infections. Antibodies against F. Tularensis have been developed, e.g. as described by Rynkiewicz, M. J. et al., 2012, Biochemistry, 51(28), 5684-5694 and Lu, Z, et al., 2013, Immunology, 140 (3), 374-389, the contents of each of which are herein incorporated by reference in their entirety.

In some embodiments, methods of the present disclosure may be used to prevent, manage and/or treat infections and complications caused by F. Tularensis.

AAV particles and methods of using the AAV particles described in the present disclosure may be used to prevent, manage and/or treat F. Tularensis related infections. As a non-limiting example, the AAV particles of the present disclosure comprise a nucleic acid sequence encoding at least one of the sequences described in Table 3 (SEQ ID NO: 1740-2141).

Toxoplasma gondii

Toxoplasma gondii is a parasitic protozoan infecting warm-blooded animals, including humans. Domestic cats and other felines are the most desired hosts for Toxoplasma gondii, as they are the only hosts where the protozoan is capable of sexual reproduction. According to CDC, more than 60 million people in the US may be infected by Toxoplasma gondii.

Toxoplasma gondii causes toxoplasmosis, which is typically asymptomatic in healthy individuals and is controlled by the natural immune system. The infection may be obtained from undercooked, contaminated food, especially pork, lamb and venison, from food contaminated by utensils, or contaminated hands, occasionally from contaminated drinking water, or by the fecal-to-oral route from cat feces. Toxoplasma gondii may also be transmitted by vertical route, especially when the protozoan is acquired during pregnancy. Children infected during or just prior to pregnancy may have eye problems, or brain damage at birth, or may develop symptoms later in their lives. Toxoplasmosis may be dangerous to individuals with a weakened immune system, such as patients with AIDS, undergoing certain chemotherapies or having organ transplants.

Toxoplasmosis may be treated with certain medications such as antibiotics called sulfadiazine and pyrimethamine, which is an anti-parasite medication used for e.g. malaria. However, resistance to both of the medications is an increasing concern. There remains a need for improved treatment methods as well as prevention therapies against Toxoplasma gondii infection. Antibodies targeting Toxoplasma gondii have been developed, as described by e.g. Graille, M. et al., 2005, J. Mol. Biol. 354 (2), 447-458, the contents of which are herein incorporated by reference in their entirety.

In some embodiments, methods of the present disclosure may be used to prevent, manage and/for treat infections and complications caused by Toxoplasma gondii.

AAV particles and methods of using the AAV particles described in the present disclosure may be used to prevent, manage and/or treat Toxoplasma gondii related infections. As a non-limiting example, the AAV particles of the present disclosure comprise a nucleic acid sequence encoding at least one of the sequences described in Table 3 (SEQ ID NO: 1740-2141).

Candida yeast

Typically, species of yeast are commensals and endosymbionts of human hosts, but may cause an infection under certain circumstances. C. albicans is a yeast belonging to the family of Saccharomycetaceae. C. albicans causes infection of the mouth characterized by white patches on the tongue, mouth and throat. The infection of the mouth is most typical with new born babies, the elderly and individuals with weakened immune system, e.g. HIV/AIDS patients. Optionally, the infection may affect the nails, leading o brittle and defected nails. Optionally, the infection may cause an infection of the vagina, leading to genital burning or uncomfortable discharge. Typically, Candida albicans infections are mild and localized. However, the infection may be severe or fatal for individuals with underlying health problems and left untreated. Invasive candidiasis refers to an infection spreading to many parts of the body, including the heart, brain, eyes, bones and/or joints. Candidemia refers to an infection where candida yeast is present in the blood stream. Severe forms of C. albicans infections affect individuals in health care environments, e.g. patients with central venous catheter, patients treated at an intensive care unit, patients undergoing antibiotic treatments, treatments for kidney failure, recovering from a surgery, and patients with chronic diseases, e.g. diabetes and/or HIV/AIDS. C. albicans is typically transmitted from mother to an infant during childbirth and it remains as a species of human's normal microflora. It may also be transmitted through the sexual transmission route. Other species of candida yeast family include, e.g., C. glabrata, C. parapsilosis, C. tropicalis, C. krusei and C. lusitaniae.

C. albicans infection may be treated with antifungal drugs, e.g. nystatin, clotrimazole, amphotericin B oral suspension) or systemic oral azoles (e.g. fluconazole, itraconazole, or posaconazole). Despite the medical therapy available, some forms of C. albicans infections are dangerous, or life-threatening. There remains a need for improved prevention, and/or treatment therapies against C. albicans infections, for example by antibody therapies. Efungumab (developed by NeuTec Pharma) is an antibody for treatment of invasive C. albicans infection.

In some embodiments, methods of the present disclosure may be used to prevent and/or treat C. albicans infections.

AAV particles and methods of using the AAV particles described in the present disclosure may be used to prevent, manage and/or treat C. albicans related infections. As a non-limiting example, the AAV particles of the present disclosure comprise a nucleic acid sequence encoding at least one of the sequences described in Table 3 (SEQ ID NO: 1740-2141).

### Human Immunodeficiency Virus (HIV)

Human immunodeficiency virus (HIV) is a roughly spherical enveloped RNA virus belonging to the family of Retroviridae. HIV is composed of two positive single-stranded RNA copies. The viral core contains a viral capsule protein, p24, which surrounds the two single stranded RNAs and the enzymes for HIV replication. The viral envelope consists of two lipid layers, the outer layer glycoprotein 120 (gp 120) and the transmembrane glycoprotein 41 (gp41). Gp120 attached to the host cell whereas gp41 has a role in the cell fusion process. For replication, the virus needs a host cell and the RNA first transcribes into DNA by enzyme reverse transcriptase. HIV infects the CD4 lymphocyte (T cell) leading to depletion of CD4+ T cells and loss of CD4+ T-cell function, as infected cell loses its function and converts to a HIV-replicating cell. (see, e.g. Okoye and Picker, 2013, Immunol Rev.; 254(1): 54-64, and references therein). Additionally, HIV infection leads to B lymphocyte (B cell) hyper-activation and dysfunction (see, e.g. Moir and Fauci, 2009, Nat Rev Immunol.; 9(4): 235-245, and references therein). The virus may be transmitted through sexual transmission route, vertical transmission route, iatrogenic (medical procedure) route, or in direct contact with certain body fluids with high concentration of HIV, including e.g. blood, breast milk, semen, vaginal, and rectal secretions. Two types of HIV (HIV-1 and HIV-2) have been identified. HIV-1 has higher infectivity and has spread around the globe whereas HIV-2 is more localized to West Africa. According to CDC, there is about 36.9 million people in the world with HIV/AIDS with about 2 million cases arising every year. The infection is most abundant in Sub-Saharan Africa.

In acute HIV infection stage, within 2.4 weeks after infection, infected patients experience flu-like illness. In the second stage the infection is asymptomatic and the HIV replication is at low level. The second stage may last for years or decades, especially when treated with HIV medication. Eventually, HIV causes acquired immune deficiency syndrome (AIDS), which is a clinical condition characterized by severe immunosuppression attacking the CD4 cells, making individuals susceptible to life-threatening malignancies and infections. Complications associated with HIV/AIDS include common bacterial and viral infections, parasite infections, certain cancers (e.g. Kaposi's sarcoma, Non-Hodgkin's lymphoma, and angiosarcoma), progressive multifocal leukoencephalopathy (PML) and wasting.

As of today, there is no prevention therapy or cure for HIV/AIDS. However, with antiretroviral (ART) therapy, the disease may be managed for along period of time. ART therapy comprises of five classes of drugs used in different combinations to treat HIV. The drugs target the different phases of the retrovirus life-cycle. However, there remains a need for improved therapies for prevention, management and/or treatment of HIV/AIDS.

Antibodies for treatment and prevention of HIV infection have been developed. For example, commercial antibody lbalizumab (developed by Taimed Biologics Inc.) is a non-immunosuppressive monoclonal antibody binding to CD4, Anaplasma phagocytophilium inhibiting the viral entry process. As another example, suvizumab (developed by Kaktsuden, Chemo-Sero Therapeutic Research Institute) is a humanized antibody targeting the HIV-1 envelope glycoprotein GP120. As a non-limiting example, any of the antibodies in Table 3, variants or fragments thereof may be used in the treatment and/or prevention of HIV.

Antibodies neutralizing HIV-1 and HIV-1 strains have been identified, but as of today, the researchers have not been able to develop a vaccination for HIV. HIV has a capability to evolve with unusually high somatic mutation and recombination rate. So far, conventional vaccines have not succeeded in eliciting analogues of the broadly neutralizing antibodies. An alternative approach suggested involves using adeno-associated vectored gene delivery for expression of antibodies from muscle tissue (e.g. Balasz et al, 2012, Nature Letter, 481, 81-84, Balasz et al, 2014, Nat Med.; 20(3): 296-300, Saunders et al., 2015, J Virol.; 89(16):8334-45, and US Patent publication US20030219733, the contents of which are herein incorporated by reference in their entirety). The studies have demonstrated efficient and long-lasting protection from HIV infection by e.g. intravenous or mucosal surface transmission.

AAV particles and methods of using the AAV particles described in the present disclosure may be used to prevent, manage and/or treat HIV infection and AIDS. As a non-limiting example, the AAV particles of the present disclosure comprise a nucleic acid sequence encoding at least one of the sequences described in Table 3 (SEQ ID NO: 1740-2141).

### Tropical Diseases (TDs) and Vector-Borne Diseases

AAV particles and methods of using the AAV particles described in the present disclosure may be used to prevent, manage and/or treat infectious disease. As a non-limiting example, the AAV particles of the present disclosure comprise a nucleic acid sequence encoding at least one of the sequences described in Table 3 (SEQ ID NO: 1740-2141).

Plasmodium falciparum

Plasmodium falciparum (P. falciparum) is a protozoan parasite belonging to Plasmodium parasite family. P. falciparum is the main cause of malaria and responsible for nearly all death cases in malaria. P. falciparum is released to the human bloodstream through mosquito saliva. The parasite has a high rate of replication and capability to alter. P. falciparum, among other Plasmodium parasites, cause malaria, which is a mosquito borne tropical disease. The early stage symptoms include fever, headache, chills and vomiting. If not treated at the early stage, malaria can progress to a life-threatening condition involving multiple organs, resulting in skin yellowing, seizures and coma. In children, malaria may cause severe anemia, respiratory distress in relation to metabolic acidosis, and/or cerebral malaria. The disease is especially dangerous for young children, pregnant women and individuals without immunity to the disease, such as travelers from non-malaria areas. An infection may develop a partial immunity, allowing the following infections to be asymptomatic. According to the WHO, about half of world's population are at risk of malaria. Sub-Saharan Africa carries the highest density of malaria. In 2015, 88% of malaria cases and 90% of malaria deaths was in Sub-Saharan Africa. Malaria is spread by female Anopheles mosquitos and caused by 5 different parasite species, out of which Plasmodium falciparum is the most prevalent and responsible for the severe cases of malaria.

Despite tremendous efforts, there is no commercial vaccination for malaria. Traditional treatment for malaria consists of antimalarial medicine therapies, such as artemisinin-based combination therapies, which consists of artemisinin combined with antimalarial drugs such as amodiaquine, lumefantrine, mefloquine and sulfadoxine/pyrimethamine. However, drug resistance has been a serious challenge in malaria treatment. Currently resistance is common for all antimalarial medications apart from artemisinin combination therapy. The cost of artemisinin treatment is high and there remains a need for prevention therapies and improved treatment against malaria.

Due to the polymorphic nature and high replication rate of P. Falciparum, tolerance to malaria is achieved only after years of repeated infections. Antibodies for prevention and treatment of malaria have been developed. For example, antibodies against P. falciparum are taught in US Patent U.S. Pat. No. 7,811,569, in US Patent publication US20150197562 and in International Patent publication WO2014087007, the contents of each of which are incorporated herein by reference in their entirety. A need for mechanism to deliver constant, effective concentration of malaria antibody for along period is still in need. Studies by Deal et al. demonstrate results on vectored immunoprophylaxis delivery of malaria antibodies to mice (see, Deal et al. PNAS, 2014, 111(34), 12528-12532).

In some embodiments, methods of the present disclosure may be used to prevent, manage and/or treat infections and complications caused by P. falciparum.

AAV particles and methods of using the AAV particles described in the present disclosure may be used to prevent, manage and/or treat P. falciparum related infections and/or conditions. As a non-limiting example, the AAV particles of the present disclosure comprise a nucleic acid sequence encoding at least one of the sequences described in Table 3 (SEQ ID NO: 1740-2141).

### Ebola Virus

Genus of Ebola virus includes five viruses, Zaire, Reston, Sudan, Tai Forest and Bundibygyo Ebola viruses, is a negative-sense RNA virus belonging to the family of filoviridae. The West Africa outbreak has been associated with Zaire Ebola virus. The genome of Ebolavirus encodes seven genes. The glycoprotein GP gene encodes two distinct gene products: sGP which is a dimeric and secreted glycoprotein and less abundant GP, which is a trimeric-virion attached, membrane embedded envelope glycoprotein and responsible for the virus attachment, fusion and entry during infection. Ebola virus disease is a hemorrhagic fever disease caused. The early symptoms include fever, sore throat, muscular pain, followed by a diarrhea and rash. Eventually the disease will affect the liver and kidney function, and cause internal bleeding. The disease is highly fatal, as about 50% infected individuals die. The Ebola virus is transmitted by direct contact with the blood and body fluids and tissues of an infected person or an animal, most commonly a chimpanzee, gorilla, fruit bat, monkey, forest antelope and porcupine. The disease is also transmitted when handling dead bodies of infected animals or humans. Also, sexual transmittance of the disease has been suggested. The WHO has reported more than 28 000 infections and 11 000 deaths in Ebola virus disease outbreak in West Africa (2014-present), mainly affecting Guinea, Sierra Leone and Liberia.

As of today, there is no licensed treatment or prevention therapy proven to neutralize the virus. Typically, Ebola virus disease is treated with a good supportive care. A variety of blood, immunological and drug therapies are under investigation, as well as preventive vaccines undergoing evaluations. However, a demand for effective therapies for treatment and prevention of Ebola virus disease remain.

Viral surface of GP has been identified as a target for neutralizing antibodies. Antibodies targeting GP of Ebola virus have been taught, e.g. in International Patent publication WO2015127136 and Olal, D., et al., 2012, J. Virol. 86 (5), 2809-2816, the contents of each of which are incorporated herein by reference in their entirety.

In some embodiments, methods of the present disclosure may be used to prevent, manage and/or treat infections and complications caused by Ebola virus.

AAV particles and methods of using the AAV particles described in the present disclosure may be used to prevent, manage and/or treat Ebola related infections and/or conditions. As a non-limiting example, the AAV particles of the present disclosure comprise a nucleic acid sequence encoding at least one of the sequences described in Table 3 (SEQ ID NO: 1740-2141).

### Marburg Virus

Marburg virus belongs to the filoviridae family of viruses with coiled, toroid or branched structures with seven proteins. The structure of Marburg virus is similar to Ebola virus; however, the involved antigens are different. The filoviruses express a single glycoprotein on their surface. The glycoprotein is responsible for the infection, as it is involved in the attachment and entry of the viruses causing infection. Marburg virus disease is a hemorrhaging fever disease caused by Marburg virus. It is highly fatal disease and related to Ebola virus diseases. The early symptoms of the disease include severe headache and malaise. Severe hemorrhagic manifestations in later stages include bleeding from multiple sites. The Marburg virus is transmitted by direct contact with the blood and body fluids and tissues of infected persons or animals, most commonly fruit bats and monkeys. The disease is also transmitted when handling dead the bodies of infected animals or humans. Marburg virus disease is uncommon, but outbreaks typically have a high rate of fatality. According to the WHO, the death rate was as high 80% in outbreaks of 1998-2000 in Democratic Republic of Congo and 2005 in Angola.

As of today, there is no preventive or treatment therapy for Marburg virus disease. The current treatment methods include good supportive treatment. The surface glycoprotein has been a target for development of antibodies for Marburg disease vaccines and treatments. For example, International Patent publication WO2015127140, and US Patent publication US20140356354, the contents of which are incorporated herein by reference in their entirety, teach therapeutic antibodies that recognize glycoprotein of filoviruses for different strains of Marburg, as well as Ebola.

In some embodiments, methods of the present disclosure may be used to prevent, manage and/or treat infections and complications caused by Marburg virus.

AAV particles and methods of using the AAV particles described in the present disclosure may be used to prevent, manage and/or treat Marburg related infections and/or conditions. As a non-limiting example, the AAV particles of the present disclosure comprise a nucleic acid sequence encoding at least one of the sequences described in Table 3 (SEQ ID NO: 1740-2141).

West Nile virus

West Nile virus (WNV) is a positive-stranded RNA of the flavivirus genome and member of the Japanese encephalitis serocomplex of flaviviruses, (see Throsby, M., J. Virol. 80 (14), 6982-6992 (2006)). Two lineages of the virus have been identified. The genome of the virus encodes a single polyprotein producing three structural proteins, capsid C, precursor membrane prM and envelope E as well as seven nonstructural proteins. WNV causes mosquito-borne infections with a variety of manifestations. Tough about 80% of WNV infections are symptomless and not harmful, in certain cases, the disease may lead to fatal neurological diseases, Infection of MNV may lead to a West Nile fever, which causes flu-like symptoms accompanied by high fever, headache, chills, excessive sweating, fatigue, weakness, swollen lymph nodes, and joint pains. Infection by MNV may also occur as cutaneous manifestations, including rashes that may include punctate erythematous, macular and popular eruptions. West Nile infections may also affect the central nervous system resulting in West Nile neuroinvasive diseases, including meningitis, encephalitis, meningoencephalitis and poliomyelitis-like syndrome. These neuroinvasive forms of NWV infections occur in only about 1% of infections, but they may be life-threatening. WNV is commonly found in Africa, Europe, the Middle East, North America and West Asia. WNV is typically transmitted to humans and other mammals by mosquitos and is maintained in nature in a cycle involving transmission between birds and mosquitoes. WNV is carried by different types of mosquitos, dependent on geographical distribution. Transmission to humans may also occur from birds, horses or other humans.

As of today, there is no specific treatment or prevention therapy for MNV infections. Current methods of treatment include good supportive care. Due to severity of some of the manifestations, there remains a need for such therapies. Envelope E has been a target of most antibody related studies. Antibodies targeting M and the first non-structural protein have also been investigated. As an example, Thorsby et al., 2006, J. Virol. 80 (14), 6982-6992, the contents of which are incorporated herein by reference in their entirety, teaches antibodies binding to E and prM proteins. US Patents U.S. Pat. Nos. 8,663,950 and 7,527,973, the contents of each of which are incorporated herein by reference in their entirety, teach antibodies binding to E protein of WNV.

In some embodiments, methods of the present disclosure may be used to prevent, manage and/or treat infections and complications caused by West Nile virus.

AAV particles and methods of using the AAV particles described in the present disclosure may be used to prevent, manage and/or treat West Nile virus related infections and/or conditions. As a non-limiting example, the AAV particles of the present disclosure comprise a nucleic acid sequence encoding at least one of the sequences described in Table 3 (SEQ ID NO: 1740-2141).

### Yellow Fever Virus

Yellow fever virus is an enveloped RNA virus belonging to the Flavivirus family. Yellow fever, also known as Yellow Jack, Yellow Plague or Bronze John, is a mosquito-borne viral hemorrhagic disease. In most cases, the symptoms include fever, headache, chills, loss of appetite, nausea, and muscle pain. In some occasions, the disease progresses to a second stage which includes fever accompanied by abdominal pains, liver damage resulting in jaundice, kidney problems and/or bleeding. The disease is spread primarily by Aedes and Haemogogus type mosquitos. The disease is most typical in tropical environments. According to the WHO, there are 200 000 annual cases of yellow fever resulting in 30 000 deaths mainly in Africa and Latin America. 90% of cases occur in Africa.

Preventive live-attenuated vaccines for yellow fever are available. However, concern related to post-vaccine adverse events has decreased the popularity of the vaccines. The vaccination is not recommended to infants younger than 9 months, pregnant women and individuals with an immune deficiency. As of today, there is no specific treatment for yellow fever. Current methods for treatment involve with supportive care to treat dehydration, respiratory failure and fever. There is a need for improved prevention and treatment therapies against yellow fever virus.

Envelope E glycoprotein of yellow fever virus has been identified as a potential target for antibody therapies. Neutralizing antibodies for yellow fever virus have been reported by Thibodeaux, B A. et al, 2012, Antiviral Res. 94 (1), 1-8 and Daffis, S. et al., 2005, Virology, 337(2), 262-272, the contents of each of which are incorporated herein by reference in their entirety.

In some embodiments, methods of the present disclosure may be used to prevent, manage and/or treat infections and complications caused by yellow fever virus.

AAV particles and methods of using the AAV particles described in the present disclosure may be used to prevent, manage and/or treat yellow fever virus related infections and/or conditions. As a non-limiting example, the AAV particles of the present disclosure comprise a nucleic acid sequence encoding at least one of the sequences described in Table 3 (SEQ ID NO: 1740-2141).

### Japanese Encephalitis Virus

Japanese encephalitis virus is an enveloped positive sense single-stranded RNA virus belonging to Flavivirus family and closely related to St. Louis encephalitis and West Nile virus. The virus causes Japanese encephalitis, also known as Japanese B encephalitis. In majority of cases, the disease is symptomless. However, in less than 1% of infections, the disease leads to a life-threatening encephalitis. The early stage symptoms include fever, headache and malaise. As the disease progresses into an acute encephalitis, the symptoms include neck rigidity, cachexia, hemiparesis, convulsions and fever, accompanied by lifelong neurological problems such as deafness, and/or mental retardation. The disease is transmitted to humans via mosquitos of the Culex species. The virus exists in a transmission cycle between mosquitos, pigs, and water birds. The disease affects 24 countries in the South-East Asia and Western Pacific. According to the WHO, an estimated 68 000 clinical cases are reported annually, with case-fatality rate as high as 30%. Major outbreaks of the disease occur every 2-15 years.

The disease may be prevented by a vaccination, most common vaccination being alive attenuated vaccine. In general, the vaccines initially show high effectiveness, but the protection decreases over time. As of today, there is no specific treatment for the disease. Current treatment therapies include good supportive care. There remains a need for longer lasting, improved prevention therapies, and treatment for Japanese encephalitis virus infections.

Antibodies for treatment of Japanese encephalitis have been developed. For example, Hsieh et al. teach antibodies that target cellular receptors and interrupts their function in flavivirus infections in US Patent publication US20080292644, the contents of which are incorporated herein by reference in their entirety.

In some embodiments, methods of the present disclosure may be used to prevent, manage and/or treat infections and complications caused by Japanese encephalitis virus.

AAV particles and methods of using the AAV particles described in the present disclosure may be used to prevent, manage and/or treat Japanese encephalitis virus related infections and/or conditions. As a non-limiting example, the AAV particles of the present disclosure comprise a nucleic acid sequence encoding at least one of the sequences described in Table 3 (SEQ ID NO: 1740-2141).

St. Louis Encephalitis Virus

St. Louis encephalitis virus is a positive-stranded RNA virus and member of the Flavivirus family and closely related to Japanese encephalitis virus. St. Louis encephalitis is a mosquito-borne disease caused by the virus. In majority of cases, the disease is symptomless. However, in less than 1% of the cases, the disease may lead to encephalitis, which may be life-threatening, especially for the elderly. The early stage symptoms include fever, headache, dizziness, malaise and nausea. If the disease progresses to the central nervous system, symptoms include stiff neck, confusion, disorientation, dizziness, tremor and unsteadiness, and in severe cases coma or even death. St. Louis encephalitis virus is transmitted to humans through Culex mosquitos. The virus exists in a transmission cycle between mosquitos and birds. The disease mainly affects the USA, especially eastern and central states. The disease has also spread to Canada and Mexico.

As of today, there is no vaccine or specific treatment for St. Louis encephalitis. Current treatment therapies include good supportive care. There is a demand for preventive and treatment therapies for the disease. Neutralizing antibodies for St. Louis encephalitis virus have been reported in Thibodeaux, B. A., et al, 2012, Antiviral Res. 94 (1), 1-8 and Daffis, S. et al., 2005, Virology 337 (2), 262-272, the contents of which are incorporated herein by reference in their entirety.

In some embodiments, methods of the present disclosure may be used to prevent, manage and/for treat infections and complications caused by St. Louis encephalitis virus.

AAV particles and methods of using the AAV particles described in the present disclosure may be used to prevent, manage and/or treat St. Louis encephalitis virus related infections and/or conditions. As a non-limiting example, the AAV particles of the present disclosure comprise a nucleic acid sequence encoding at least one of the sequences described in Table 3 (SEQ ID NO: 1740-2141).

### Neglected Tropical Diseases (NTDs)

AAV particles and methods of using the AAV particles described in the present disclosure may be used to prevent, manage and/or treat infectious disease. As a non-limiting example, the AAV particles of the present disclosure comprise a nucleic acid sequence encoding at least one of the sequences described in Table 3 (SEQ ID NO: 1740-2141).

Neglected Tropical diseases (NTDs) are a diverse category of communicable diseases present in tropical and subtropical environments. NTDs affect more than one billion people in about 150 countries. NTDs are a significant public health problem costing the involved developing economies billions of dollars annually. The diseases affect mostly the populations with inadequate sanitation, and those in contact with infectious vectors, domestic animals and livestock. In May 2013, the 66^{th} WHO Assembly announced resolution WHA66.12 to integrate measures and plan investments to improve the wellbeing of populations affected by NTDs. NTDs include Buruli ulcer, Chagas disease, Dengue and Chikungunya, Dracunculiasis (guinea-worm disease), Echinococcosis, Endemic treponematoses (Yaws), Foodborne trematodiases, Human African trypanosomiasis (sleeping sickness), Leishmaniasis, Leprosy (Hansen disease), Lymphatic filariasis, Onchocerciasis (river blindness), Rabies, Schistosomiasis, Soil-transmitted helminthiases, Taeniasis/Cysticercosis and Trachoma.

### Chikungunya Virus

Chikungunya virus is an arbovirus belonging to the Togoviridae family. The genome is a single-strand RNA molecule encoding four non-structural and three structural glycoproteins (C, E1, E2) (see, e.g. Caglioti et al., 2013, New Microbiol.; 36(3):211-27, and references therein). Chikungunya fever is a mosquito-borne disease caused by chikungunya virus. The symptoms include a fever lasting 2-7 days, rash and flu-like symptoms accompanied by a joint pain that may last for weeks, months or even years. The disease may be dangerous for the elderly and individuals with chronic medical problems. Chikungunya virus is spread by Aedes albopictus and Aedes aegypti. Outbreaks of chikungunya fever have occurred in Africa, Asia, Europe and Indian and Pacific Oceans, and more recently in islands in the Caribbean. As an example, according to the WHO, an outbreak of 1.9 million cases in India, Indonesia, Maldives, Myanmar and Thailand since 2005 has been reported. More recently, as of April 2015 more than million cases have reported in Caribbean Islands, Latin American countries and the United States.

As of today, there is no specific treatment or vaccination for chikungunya fever. The disease is typically treated with supportive care therapy, as well as anti-inflammatory drugs and medicines to relieve the symptoms. Research and development on vaccinations has been done but none has been approved for commercial use so far. Antibodies for detection and treatment of Chikungunya have been developed. E.g. fully human antibodies binding to an epitope located in an antigenic site of the chikungunya virus E1 and E2 envelope proteins were in US Patent Publication US20130189279, the contents of each of which are incorporated herein by reference in their entirety.

In some embodiments, methods of the present disclosure may be used to prevent, manage and/or treat infections and complications caused by chikungunya virus.

AAV particles and methods of using the AAV particles described in the present disclosure may be used to prevent, manage and/or treat chikungunya virus related infections and/or conditions. As a non-limiting example, the AAV particles of the present disclosure comprise a nucleic acid sequence encoding at least one of the sequences described in Table 3 (SEQ ID NO: 1740-2141).

### Dengue Virus

Dengue virus belongs to the family of Flaviviridae, genus of Flavivirus. It is an enveloped, positive strand RNA virus containing two integral membrane proteins envelope (E) and premembrane (prM). Dengue virus is closely related to e.g. Yellow fever, West Nile virus and St. Louis and Japanese encephalitis viruses. There are five serotypes of the virus that can cause dengue fever, which is a mosquito-borne tropical disease. Neutralizing antibodies target the protein E as it binds to the cellular receptors and mediates the viral entry into cells. Infection with a serotype may produce a lifelong immunity to that serotype but no long-term immunity against other serotypes, (see e.g., Wahala and de Silva, 2011, Viruses.; 3(12): 2374-2395, and references therein). In fact, an infection by a second serotype may lead to a more severe form of disease, due to the complexity of the antibody respond and possible antibody dependent enhancement (ADE), which hypothesizes that weakly neutralizing antibodies from the first infection bind to the second serotype and enhance the infection. The symptoms of dengue fever are similar to flu, including fever, headache, muscle and joint pain and skin rash. The disease may also manifest as a potentially lethal complication called severe dengue, also known as dengue hemorrhagic fever. The disease may be dangerous to individuals with chronic diseases, such as diabetes or asthma, or children and the elderly. Dengue virus is spread by several mosquito species, out of which Aedes aegypti is the most common. Dengue may also be transmitted via infected blood or organ donation or by the vertical transmission route. According to the WHO, the estimated number of dengue infections annually could be as high as 390 million.

As of today, there is no specific treatment or prevention therapy for dengue fever. Antibodies targeting dengue virus have been developed. As an example, antibodies neutralizing four serotypes of dengue virus have been in US Patent publication US20150225474, US20150218255 and in US Patent U.S. Pat. No. 9,073,981, the contents of each of which are incorporated herein by reference in their entirety.

In some embodiments, methods of the present disclosure may be used to prevent, manage and/or treat infections and complications caused by dengue virus.

AAV particles and methods of using the AAV particles described in the present disclosure may be used to prevent, manage and/or treat Dengue virus related infections and/or conditions. As a non-limiting example, the AAV particles of the present disclosure comprise a nucleic acid sequence encoding at least one of the sequences described in Table 3 (SEQ ID NO: 1740-2141).

Trypanosoma cruzi

Trypanosoma Cruzi(T. cruzi) is a species of parasitic euglenoid protozoan. T. cruzi causes Chagas disease, also known as American trypanosomiasis, which is a tropical parasitic disease. The symptoms of Chagas disease at the early stage include fever, swollen lymph nodes, headaches or local swelling at the site of bite. The chronic phase of Chagas starts after 8-12 weeks, which may be symptomless, or include enlargement of the ventricles of the heart, which may result in heart failure, or to an enlarged esophagus or enlarged colon. The severity of Chagas disease varies from almost unnoticeable to fatal. Chagas disease is spread by an insect vector triatomine bug. These bugs get infected with T. cruzi by feeding on the blood of an infected human or animals, and they spread it further by bites and ingestion of blood. The triatomine bug is also known as a “kissing bug” referring to its tendency to feed on people's faces. T. cruzi may also be transmitted through blood transfusions or through breast milk. Chagas disease is present mainly in 12 Latin American countries, but has also spread to other continents. According The WHO, over 10 000 people die every year from Chagas disease, and 25 million people are in the risk of infection.

As of today, there is no specific prevention or treatment therapy for Chagas disease. The traditional therapies for Chagas have been involved with attempts to kill the parasite and treatment of the symptoms. For example, azole and nitro-derivative drugs have been used, but have not been successful in removal of the parasite fully. Other mechanisms to treat the disease have been under research. After infection in mammals, the parasite incorporates a charged carbohydrate (sialic acid) to survive to the chronic phase of the disease. To do so, the parasite scavenged sialic acid it from the host's sialoglycoconjugates, through a transglycosylation reaction catalyzed by an enzyme called trans-sialidase. The trans-sialidase has been identified as a potential target for drug development. Buschiazzo et al. have reported an antibody inhibiting the T. cruzi trans-sialidase enzyme providing an antibody therapy mechanism for Chagas disease (see, Buschiazzo et al., 2012, PLoS Pathol. 8(1), E1002474, and references therein).

In some embodiments, methods of the present disclosure may be used to prevent, manage and/or treat infections and complications caused by Chagas disease.

AAV particles and methods of using the AAV particles described in the present disclosure may be used to prevent, manage and/or treat Chagas disease. As a non-limiting example, the AAV particles of the present disclosure comprise a nucleic acid sequence encoding at least one of the sequences described in Table 3 (SEQ ID NO: 1740-2141).

### Rabies Virus

Lyssaviruses are a genus of RNA viruses belonging to the family of Rhabdoviridae. Rabies virus is a neurotropic virus with cylindrical morphology. After infection, rabies virus enters the peripheral nervous system, and further to central nervous system by retrograde axonal transport. Rabies virus and Australian bat lyssavirus cause rabies. Rabies affects humans and warm-blooded animals. The early stage symptoms include flu-like signs, but later the disease manifests as paralysis, anxiety, insomnia, abnormal behavior, hallucinations. Humans and animals infected may also experience hydrophobia, “fear of water”, which is considered a characteristic symptom of the disease. Eventually the disease affects the central nervous system and brain, causing death. Humans are typically infected by being bitten, scratched or licked by an animal with the disease. Most commonly the infection is by dogs. Whereas efficient vaccination programs for animals have been able to reduce or even eliminate rabies in developing countries, the disease still affects poor population mainly in Africa and Asia. According to the WHO, post-bite treatment and vaccination is provided for 15 million people annually.

Rabies is a vaccine-preventable disease and especially systematic vaccination of dogs has been a cost-effective strategy for prevention of rabies. Post-exposure prophylaxis (PEP), the treatment of bite victims immediately after the exposure, includes local treatment of the wound, rabies vaccination and administration of rabies immunoglobulin. Though efficient vaccines for rabies have been developed, there remains a need for treatment/or management of rabies to prevent death after rabies virus has entered the central nervous system (see, e.g., Hicks et al., 2012, Clin Exp Immunol.; 169(3): 199-204, and references therein). The genome of rabies virus codes for five viral proteins. Out of the five, G protein, which is an external surface glycoprotein, forms protrusions that cover the outer surface of the virion envelope and is known to induce neutralizing antibodies. Also, nucleoprotein (N) molecules and the phospho-protein (NS) participate in immune responses. G protein has been the target of antibody developments. For example, therapeutic antibodies against rabies virus are taught in US Patents U.S. Pat. Nos. 7,071,319, 6,890,532, and 9,005,624, the contents of each of which are incorporated herein by reference in their entirety.

In some embodiments, methods of the present disclosure may be used to prevent, manage and/or treat infections and complications caused by rabies virus.

AAV particles and methods of using the AAV particles described in the present disclosure may be used to prevent, manage and/or treat rabies virus related infections and/or conditions. As a non-limiting example, the AAV particles of the present disclosure comprise a nucleic acid sequence encoding at least one of the sequences described in Table 3 (SEQ ID NO: 1740-2141).

### Foodborne Illnesses and Gastroenteritis

AAV particles and methods of using the AAV particles described in the present disclosure may be used to prevent, manage and/or treat infectious disease. As a non-limiting example, the AAV particles of the present disclosure comprise a nucleic acid sequence encoding at least one of the sequences described in Table 3 (SEQ ID NO: 1740-2141).

Foodborne illnesses, also known as food poisoning, are a common and costly public health problem. The illnesses are typically transmitted by the fecal-oral-route. The transmission to humans is by consuming contaminated food or beverage. More than 250 different foodborne diseases, mostly infections caused by viruses, bacteria, parasites or fungus, are identified by the CDC. CDC estimates that approximately 48 million individuals are affected by foodborne illnesses annually in the United States. Gastroenteritis is an inflammation of the gastrointestinal tract involving stomach and small intestine. Gastroenteritis is also caused by an infection caused by viruses, bacteria, parasites or fungus. The transmission to humans is by person-to-person contact, or by consuming contaminated food or beverage. Foodborne illnesses and gastroenteritis have similar symptoms including diarrhea, vomiting, abdominal pain, dehydration. In some cases, the diseases may require hospitalization or be fatal. Both illnesses are best prevented by proper hand hygiene, proper hygiene while preparing food, treatments to kill bacteria such as pasteurizing, cooking or heating food, and proper methods to store food.

### Rotavirus

Rotavirus is a double-stranded RNA virus belonging to the family of Reoviridae. The rotavirus genome consists of 10 segments coding for a single protein, and segment 11 coding for two proteins. The virions are non-enveloped, triple-layered and icosahedral in structure (see, e.g. Alyegbo et al., 2013, Plos One 8, 61101, and references therein). The virus is spread by the fecal-oral-route. Rotavirus is very common especially among infants and young children and spreads easily. Almost all children worldwide are infected with rotavirus by the age of 5, and the disease leads to death of half a million children annually. Rotavirus causes rotavirus gastroenteritis with symptoms including nausea, vomiting, diarrhea and fever. Rotavirus is associated with dehydration. The disease is milder in adults and more severe in young children, infants and the elderly. Though infection does not provide full immunity to the virus, the first infection is typically the most severe in symptoms.

As of today, there is no specific treatment rotavirus infections. Present treatment includes good supportive care including drinking of fluids to prevent dehydration. In severe cases, the rotavirus gastroenteritis requires hospital care e.g. treatment with intravenous fluids. Vaccines for prevention of the disease have been developed and CDC recommends rotavirus vaccination for infants as part of the routine vaccinations. There remains a need for medical treatment therapies for the infection. Development has been done in the field of antibodies. E.g. Alyegbo et al., in Plos One 8, 61101 (2013, teach antibodies targeting the intermediate capsid layer of VP6 of the triple-layered particle and Frenken et al. teach anti-rotavirus antibodies in US Patent U.S. Pat. No. 8,105,592, the contents of which are incorporated herein by reference in their entirety.

In some embodiments, methods of the present disclosure may be used to prevent, manage and/or treat infections and complications caused by rotavirus.

AAV particles and methods of using the AAV particles described in the present disclosure may be used to prevent, manage and/or treat rotavirus related infections and/or conditions. As a non-limiting example, the AAV particles of the present disclosure comprise a nucleic acid sequence encoding at least one of the sequences described in Table 3 (SEQ ID NO: 1740-2141).

### Norwalk Virus/Norovirus

Norwalk virus, also known as winter vomiting bug, is the only member of genus norovirus belonging to the family of Caliciviridae. Norwalk virus is a single-stranded RNA with three open-reading frames that encode a polyprotein precursor to non-structural proteins, and two polypeptides of different sizes (see e.g. Jiang et al., 1993, Virology; 195(1):51-61, and references therein). Norwalk virus is spread by the fecal-oral-route. Norwalk virus is extremely contagious and can be transmitted through contaminated food or drink, touching contaminated surfaces or objects or from a contact with an infected individual. The Norwalk virus causes an inflammation of stomach and/or intestines. The symptoms associated with the infection include stomach pain, nausea, vomiting and diarrhea. The disease can be dangerous, especially for your children or young adults. According to CDC, every year 19-21 million infections occur leading to 570-800 deaths in the US.

As of today, there is no vaccine or specific treatment for Norwalk virus associated gastroenteritis. Antibodies for prevention and treatment of Norwalk virus have been developed. For example, International Patent publication WO2014126921 and WO2014183052, the contents of each of which are incorporated herein by reference in their entirety, teach neutralizing antibodies binding to the polypeptides of Norwalk virus.

In some embodiments, methods of the present disclosure may be used to prevent, manage and/or treat infections and complications caused by Norwalk virus.

AAV particles and methods of using the AAV particles described in the present disclosure may be used to prevent, manage and/or treat Norwalk virus related infections and/or conditions. As a non-limiting example, the AAV particles of the present disclosure comprise a nucleic acid sequence encoding at least one of the sequences described in Table 3 (SEQ ID NO: 1740-2141).

Campylobacter jejuni

Campylobacter jejuni(C. jejuni) is an oxidase-positive, catalase-positive, non-fermentative Gram-negative bacteria with a helical shape. The C. jejuni inhabits in the intestinal tract of animals (e.g. poultry, cattle, pigs, sheep, ostriches and shellfish), and in pets (e.g. cats and dogs). The bacteria may be transmitted to humans foodborne, e.g. when eating contaminated food or drink, such as unpasteurized milk. According to the WHO, campylobacter is the most common cause of gastroenteritis worldwide. C. jejuni causes campylobacteriosis infection. The typical symptoms include diarrhea with blood in the feces, abdominal pain, fever, headache, nausea and/or vomiting. The infection may be dangerous to young children, the elderly and individuals with immunodeficiency and is most abundant with malnourished children. C. jejuni infections have been associated with severe long-term complications such as Guillain-Barre Syndrome, inflammatory bowel disease and reactive arthritis (see, e.g., Platts-Mills and Kosek, 2014, Curr Opin infect Dis.; 27(5): 444-450, and references therein).

Typically, C. jejuni infection does not require specific treatment in addition to good supportive care. In more severe cases, in humans and in poultry, the infection has been treated with antibiotics such as fluoroquinoles and macrolides. However, spread of antibiotic-resistant strains is an increasing concern. The treatment with antibiotics is recommended in cases where the bacteria has invaded the intestinal mucosa cell and damaged the tissues, or to eliminate the carrier state. There remains a need for prevention therapies, as well as improved, non-antibiotic, therapies for treatment of the infection. Antibodies targeting C. jejuni have been taught e.g. in International Patent publication WO2014063253, the contents of which are incorporated herein by reference in their entirety.

In some embodiments, methods of the present disclosure may be used to prevent, manage and/or treat infections and complications caused by C. Jejuni.

AAV particles and methods of using the AAV particles described in the present disclosure may be used to prevent, manage and/or treat C. Jejuni related infections and/or conditions. As a non-limiting example, the AAV particles of the present disclosure comprise a nucleic acid sequence encoding at least one of the sequences described in Table 3 (SEQ ID NO: 1740-2141).

Clostridium difficile

Clostridium difficile bacteria (C. difficile) is a Gram-positive, anaerobic spore-forming bacteria belonging to the genus of Clostridium. C. difficile inhabits in the soil. C. difficile produces toxins, most commonly enterotoxin A and cytotoxin B. Toxins A and B both have a C-terminal receptor-binding domain containing repeating sequences, a central hydrophobic domain and N-terminal glucosyltranferase domain. The toxins bind to the intestinal epithelial cells leading to glucosylation of target Rho GTPases, disruption of the cytoskeleton and cell death. C. difficile toxins A and Bare a common cause C. difficile associated diarrhea and Clostridium difficile colitis, which is an inflammation of the large intestine. Typical symptoms of the colitis include flu-like symptoms, bloating, diarrhea, and/or abdominal pain. The disease may lead to dehydration, kidney failure, bowel perforation, toxic megacolon resulting in colon rupture. The elderly and individuals with a weakened immunity are more susceptible to severe and recurring infections which can be life-threatening. C. difficile is transmitted by the fecal-oral-route. Due to the ability to form heat-resistant spores, the bacteria is not killed by alcohol-based cleansers or routine surface cleaning. The bacteria may be cultured on almost any surface and survives in clinical environments, such as hospitals. C. difficile is one of the most common and severe healthcare-associated infections. According to CDC, an estimated about half a million infections occur in the United States annually. In 2011, 29, 000 deaths related to C. difficile were reported.

Currently C. difficile infections are treated with antibiotics such as vancomycin and metronidazole. However, increasing an antibiotic-resistance to the bacteria is a concern. Especially in cases of recurring infections, antibiotic treatments have an incomplete response and they disrupt the normal colonic flora. There remains a need for prevention and improved treatment therapies for the infection. Antibodies targeting C. difficile have been developed. For example, actoxumab and bezlotoxumab (developed by Medarex Inc. and the University of Massachusetts Medical School) are human monoclonal antibodies targeting C. difficile toxin A and toxin B, respectively. The antibodies may be administered as a combination for the prevention of recurring C. difficile infection.

In some embodiments, methods of the present disclosure may be used to prevent, manage and/or treat infections and complications caused by C. difficile.

AAV particles and methods of using the AAV particles described in the present disclosure may be used to prevent, manage and/or treat C. difficile related infections and/or conditions. As a non-limiting example, the AAV particles of the present disclosure comprise a nucleic acid sequence encoding at least one of the sequences described in Table 3 (SEQ ID NO: 1740-2141).

Entamoeba histolytica

Entamoeba histolytica (E. histolytica) is an anaerobic one-celled parasite protozoan belonging to the genus of Entamoeba. The active stage of the protozoan exists only in the host and in fresh feces. Cysts survive outside the host in water, soil and food in moist conditions. E. histolytica causes an infection called amebiasis, also known as amoebiasis or entamoebiasis. In majority of cases, amebiasis is symptomless. In 10-20% of individuals infected have symptoms that include loose feces, stomach pain and cramping. The severe more form of amebiasis called amebic dysentery is associated with stomach pain, blood stools and fever. In rare cases, E. histolytica invades the liver, forms an abscess and may spread to other parts of the body, such as the lungs or brain. The transmission to humans is mostly via the fecal-oral-route. The disease is typically caused by ingestion of mature cysts in contaminated food, water or via hands. The disease may also be transmitted in close person-to-person contact, e.g. sexual contact. E. histolytica infections are most common in tropical areas and especially in poor sanitary conditions. It is estimated that 50 million cases of amebiasis occur annually, leading to 100, 000 deaths.

As of today, there are no preventive vaccines for E. histolytica infections, though cellular immunity is important for the prevention of liver invasive amebiasis. Amebiasis is typically treated with amebicides, which are medicines targeting E. histolytica at specific parts of the body, e.g. the intestine tissue or liver. Optionally, the treatment may involve one or more antibiotics, as well as steroids. However, increasing antibiotic-resistance of E. histolytica is a concern. There remains a need for prevention therapy as well as for improved treatments. Antibodies targeting E. histolytica are taught in, e.g., 2009, infect Immun.; 77(1): 549-556, and Tachibana et al., 1999, Clin Diagn Lab Immunol.; 6(3):383-7, the contents of which are incorporated herein by reference in their entirety.

In some embodiments, methods of the present disclosure may be used to prevent, manage and/or treat infections and complications caused by E. histolytica.

AAV particles and methods of using the AAV particles described in the present disclosure may be used to prevent, manage and/or treat E. histolytica related infections and/or conditions. As a non-limiting example, the AAV particles of the present disclosure comprise a nucleic acid sequence encoding at least one of the sequences described in Table 3 (SEQ ID NO: 1740-2141).

Helicobacter pylori

Helicobacter pylori (H. pylori) is a Gram-negative, spiral-shaped microaerophilic bacterium. H. pylori infection is typically asymptomatic and is suggested to be transmitted through the fecal-oral route or oral-oral route. According to CDC, two-thirds of the world's population is infected with H. pylori. The infection may cause chronic active, chronic, persistent, and atrophic gastritis, duodenal and gastric ulcers and is associated with cancer. CDC reposts 25 million Americans suffering from an ulcer during their lifetime. Typical symptoms associated with ulcer are gnawing or burning pain in the epigastrium, especially between meals. Additional symptoms include nausea, vomiting, loss of appetite, internal bleeding leading to anemia and fatigue.

Typical treatment for H. pylori infection involves antibiotics. Increasing antibiotic resistance and patient noncompliance are major challenges associated with the antibiotic treatment. There remains a need for improved, non-antibiotic, treatment and prevention therapies targeting H. pylori. Antibodies targeting H. pylori infection have been developed. For example, Boren et al. teach antibodies targeting the BAbA antigen expressed by H. pylori in US patent U.S. Pat. No. 8,025,880, the contents of which are incorporated herein by reference in their entirety.

In some embodiments, methods of the present disclosure may be used to prevent, manage and/or treat infections and complications caused by H. pylori.

AAV particles and methods of using the AAV particles described in the present disclosure may be used to prevent, manage and/or treat H. pylori related infections and/or conditions. As a non-limiting example, the AAV particles of the present disclosure comprise a nucleic acid sequence encoding at least one of the sequences described in Table 3 (SEQ ID NO: 1740-2141).

### Enterotoxin B

Enterotoxin B is a toxin produced by certain strains of Gram-positive bacteria Staphylococcus aureus and is a common cause for food poisoning. Staphylococcus species thrive and produce toxins in unrefrigerated meats, dairy, and bakery products. The symptoms associated with enterotoxin B infection are severe diarrhea, nausea and intestinal cramping. The toxin may remain active in the human body after the bacteria has been killed. Enterotoxin B is a so-called superantigen. Superantigens are toxins that may activate T cells by forming a bridge between a MHC II on antigen presenting cells (APCs) and the T cell receptors (TCR). Due to binding of enterotoxin B, the T cells release large amount of cytokines leading to an inflammation and gastroenteritis. Though enterotoxin B infection is typically not life threatening, enterotoxin B has been identified as a potential chemical and biological warfare agent.

As of today, there is no specific prevention or treatment for enterotoxin B infection. Antibodies that neutralize enterotoxin B have been investigated, e.g. as described in US Patent U.S. Pat. No. 8,895,704.

In some embodiments, methods of the present disclosure may be used to prevent, manage and/or treat infections and complications caused by enterotoxin B.

AAV particles and methods of using the AAV particles described in the present disclosure may be used to prevent, manage and/or treat enterotoxin B related infections and/or conditions. As a non-limiting example, the AAV particles of the present disclosure comprise a nucleic acid sequence encoding at least one of the sequences described in Table 3 (SEQ ID NO: 1740-2141).

### Toxins

AAV particles and methods of using the AAV particles described in the present disclosure may be used to prevent, manage and/or treat infectious disease. As a non-limiting example, the AAV particles of the present disclosure comprise a nucleic acid sequence encoding at least one of the sequences described in Table 3 (SEQ ID NO: 1740-2141).

Toxins are a group of substances that are highly poisonous and dangerous to humans. Toxins are infectious agents in form of bacteria, viruses, fungi, proteins, and other chemical and/or biological substances. Toxins may lead to fatal conditions. Toxins are produced by nature, and may be produced synthetically. Exposure to toxins may be unintentional and occur when in contact with toxic plants, or contaminated food, water, livestock or animals. Due to the life-threatening impact of toxins, they are considered to be potential biological and/or chemical warfare agents that may be applied as weapons of mass destruction in war field. They also impose a threat to be used as means for terrorist attacks.

### Ricin

Is a naturally occurring carbohydrate-binding lectin protein produced by castor oil plant growing in Eastern Africa, India, Southeastern Mediterranean basin area, and in tropical regions. Ricin may also be manufactured from the waste products when processing castor beans. Ricin has a globular structure with two toxin chains, chain A and chain B, which both need to be present for the cytotoxic affect. Ricin kills cells by inhibiting protein synthesis. Chain B penetrates to the cell whereas the disulfide bond joining chain A to chain B lectin has an affinity to bind to cell surface carbohydrates, (see, e.g. Friedman and Rasooly, 2013, Toxins (Basel); 5(4): 743-775). Ricin is highly toxic to humans with median lethal dose (LD₅₀) of 22 micrograms per kilogram of body weight. The exposure to Ricin may be by inhaling, ingestion or by injection. The symptoms are dependent of the method of exposure. When inhaled, ricing causes severe inflammation of the lungs, causing would has symptoms including coughing, difficulty breathing, muscle ache and nausea. When ingested, ricin induces internal bleeding of the stomach and intestines leading to pain, vomiting and bloody diarrhea, and eventual failure of the kidneys, liver and spleen. When injected, ricin induces failure of the muscles and lymph nodes, and eventually failure of the liver, kidney and spleen. There is no known treatment for Ricin poisoning.

Unintentional poisoning by Ricin is uncommon. However, Ricin is a potential biological and chemical warfare agent creating a need for treatment and prevention therapies for ricin poisoning. Antibodies targeting ricin have been developed, as described e.g. in International publication WO2015100409, the contents of which are herein incorporated by reference in their entirety.

In some embodiments, methods of the present disclosure may be used to prevent, manage and/or treat infections and complications caused by ricin.

AAV particles and methods of using the AAV particles described in the present disclosure may be used to prevent, manage and/or treat Ricin related infections and/or conditions. As a non-limiting example, the AAV particles of the present disclosure comprise a nucleic acid sequence encoding at least one of the sequences described in Table 3 (SEQ ID NO: 1740-2141).

Bacillus anthracis

Bacillus anthracis is a Gram-positive, rod-shaped bacterium causing anthrax disease (see, e.g. Spencer, 2003, J Clin Pathol.; 56(3): 182-187, and references therein). Most animals, especially herbivores, are susceptible to infection of Bacillus anthracis. Anthrax may be infected via respiratory exposure, skin contact or eating contaminated food, in most cases meat. Inhaled anthrax causes flu-like symptoms, pneumonia and severe respiratory collapse. Gastrointestinal anthrax causes severe diarrhea, acute inflammation of the intestinal tract, and vomiting of blood. Skin exposure to the bacteria will lead to boil-like skin lesions forming an ulcer with black center. Typically, infection to humans occurs by eating contaminated meat or while handling infected animals or their product, such as skin, wool or meat. Bacillus anthracis is a potential biological warfare agent. In 2001, weeks following the September 11 terrorist attacks, letters containing Bacillus anthracis were mailed to news media offices and two U.S. Senators resulting in death of five people and infected many more.

Anthrax may be treated with antibiotics, such as penicillin and amoxicillin, and may be prevented by vaccines, developed both for humans and animals. However, due to increased threat of biological warfare and terrorism, improved methods of treatment are in demand. Anthrax may also be treated by antibody therapy. For example, Raxibacumab (developed by Cambridge Antibody Technology and Human Genome Sciences) is an antibody for the prophylaxis and treatment of inhaled anthrax.

In some embodiments, methods of the present disclosure may be used to prevent, manage and/or treat infections and complications caused by Bacillus anthracis.

AAV particles and methods of using the AAV particles described in the present disclosure may be used to prevent, manage and/or treat Bacillus anthracis related infections and/or conditions. As a non-limiting example, the AAV particles of the present disclosure comprise a nucleic acid sequence encoding at least one of the sequences described in Table 3 (SEQ ID NO: 1740-2141).

### Shiga Toxin and Shiga-Like Toxin

Shiga toxin, including two major types Stx1 and Stx2, is a toxin produced by Shigella dysenteriae, a rod-shaped bacteria belonging to bacterial genus Shigella. Shiga toxin inhibits protein synthesis within cells. The toxin enters cell via a marcopinosome and inhibits the protein synthesis by cleaving a specific nucleobase RNA of the 60S subunit of ribosome. Shiga-like toxins 1 and 2 are structurally similar to Stx1 and Stx2 and are produced by enterohemorrhagic strains of Escherichia coli (EHEC) strains. (see, e.g. Friedman and Rasooly, Toxins (Basel). 2013 April; 5(4): 743-775). EHEC type 0157 is the most common pathogen causing E. Coli outbreaks in the US. Stx2 is considered to be orders of magnitude more toxic that Stx1. The severity of Shiga toxin foodborne illnesses range from mild diarrhea to a life-threatening complication known as hemolytic uremic syndrome (HUS). HUS is a disease associated with hemolytic anemia, acute kidney failure and low platelet count. Cattle is the major source or infection to humans, but the disease may be spread by birds or pigs. Shiga infection is typically obtained from contaminated food or drink, such as meat, unpasteurized milk, or contaminated water, or by contact with cattle. Shiga toxin and Shiga-like toxins considered to be potential chemical and biological warfare agents.

As of today, there is no prevention therapy or specific treatment for Shiga and Shiga-like toxins. Recent developments have been made in antibody therapy of Shiga toxin induced HUS. For example, SHIGAMAB™ (developed by Bellus Health Inc.) is a monoclonal antibody for treatment of Shiga toxin induced HUS.

In some embodiments, methods of the present disclosure may be used to prevent, manage and/or treat infections and complications caused by Shigella dysenteriae.

AAV particles and methods of using the AAV particles described in the present disclosure may be used to prevent, manage and/or treat Shigella dysenteriae related infections and/or conditions. As a non-limiting example, the AAV particles of the present disclosure comprise a nucleic acid sequence encoding at least one of the sequences described in Table 3 (SEQ ID NO: 1740-2141).

### Botulinum Toxins

Botulinum toxins are neurotoxins produced by Clostridium bacteria and they cause a disease called botulism which is characterized by weakness, problems in vision, tiredness, and problems with speech, followed by weakness of the arms, chest muscles and legs. Botulism may be fatal. There are seven different botulinum neurotoxins with a four-domain structure varying in antigenic properties and interactions with intracellular targets. L-chain enters the cytosol, cleaves the synaptosome protein and blocks neurotransmitter release resulting in peripheral neuromuscular blockade and flaccid paralysis in humans. (see, e.g. Friedman and Rasooly, Toxins (Basel). 2013 April; 5(4): 743-775) Botulinum neurotoxins are highly dangerous to humans, serotype A having a median lethal dose (LD₅₀) of 0.8 micrograms for a human of 70 kg weight. The bacteria is common in soil and water and may produce the botulinum toxins when exposed to low oxygen levels and certain temperatures. Outbreaks of foodborne botulism occur occasionally. Most susceptible to contamination by botulinum are baked products, fresh mussels, canned fruit and vegetables. Infant botulism occurs when the toxins are produced and released by bacteria in the infant's intestines. Botulism may also occur in wounds where the bacteria in the absence of oxygen produces and releases the toxins. Wound botulism is most common in cases where contaminated needles are used for injection. Botulinum toxins are potential biological and chemical warfare agents.

As of today, there is no prevention therapy for botulism. Botulism may be treated with antitoxins that block the circulation of toxins in the blood and prevent worsening of the disease. However, the antitoxins are expensive and not easily available. In cases of wound botulism, the area infected may be removed surgically. Additionally, good supportive care therapy is applied. There remains a need for therapies to prevent and treat botulism. Antibodies targeting botulinum toxins are developed, as described e.g. in US Patent publication US20130058962, and International publication WO2015100409, the contents of each of which are herein incorporated by reference in their entirety.

In some embodiments, methods of the present disclosure may be used to prevent, manage and/or treat infections and complications caused by botulinum toxins.

AAV particles and methods of using the AAV particles described in the present disclosure may be used to prevent, manage and/or treat botulinum toxin related infections and/or conditions. As a non-limiting example, the AAV particles of the present disclosure comprise a nucleic acid sequence encoding at least one of the sequences described in Table 3 (SEQ ID NO: 1740-2141).

### Therapeutic Applications: Non-Infectious Disease

The present disclosure additionally provides a method for treating non-infectious diseases and/or disorders in a mammalian subject, including a human subject, comprising administering to the subject any of the AAV particles or pharmaceutical compositions of the disclosure. In some embodiments, non-infectious diseases and/or disorders treated according to the methods described herein include, but are not limited to, Parkinson's Disease (PD), Dementia with Lewy Bodies (DLB), Multiple System Atrophy (MSA), Decreased muscle mass, Spinal muscular atrophy (SMA) Alzheimer's disease (AD), Amyotrophic lateral sclerosis (ALS), Huntington's Disease (HD), Multiple sclerosis (MS), Stroke, Migraine, Pain, Neuropathies, Psychiatric disorders including schizophrenia, bipolar disorder, and autism, Cancer, ocular diseases, systemic diseases of the blood, heart and bone, Immune system and Autoimmune diseases and Inflammatory diseases.

In some embodiments, methods of treating non-infectious diseases and/or disorders in a subject in need thereof may comprise the steps of: (1) deriving, generating and/or selecting an antibody, antibody-based composition or fragment thereof that targets the antigen of interest; (2) producing an AAV particle with a viral genome that includes a payload region encoding the selected antibody of (1); and (3) administering the AAV particle (or pharmaceutical composition thereof) to the subject.

The present disclosure provides a method for administering to a subject in need thereof, including a human subject, a therapeutically effective amount of the AAV particles of the disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

### Therapeutic Applications: Non-Infectious Disease

The present disclosure additionally provides a method for treating non-infectious diseases and/or disorders in a mammalian subject, including a human subject, comprising administering to the subject any of the AAV particles or pharmaceutical compositions of the disclosure. In some embodiments, non-infectious diseases and/or disorders treated according to the methods described herein include, but are not limited to, Parkinson's Disease (PD), Dementia with Lewy Bodies (DLB), Multiple System Atrophy (MSA), decreased muscle mass, spinal muscular atrophy (SMA) Alzheimer's disease (AD), Amyotrophic lateral sclerosis (ALS), Huntington's Disease (HD), multiple sclerosis (MS), stroke, migraine, pain, neuropathies, psychiatric disorders including schizophrenia, bipolar disorder, and autism, cancer, ocular diseases, systemic diseases of the blood, heart and bone, immune system and autoimmune diseases and inflammatory diseases.

In some embodiments, methods of treating non-infectious diseases and/or disorders in a subject in need thereof may comprise the steps of: (1) deriving, generating and/or selecting an antibody, antibody-based composition or fragment thereof that targets the antigen of interest; (2) producing an AAV particle with a viral genome that includes a payload region encoding the selected antibody of (1); and (3) administering the AAV particle (or pharmaceutical composition thereof) to the subject.

The present disclosure provides a method for administering to a subject in need thereof, including a human subject, a therapeutically effective amount of the AAV particles of the disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

### Cancer and Immunoinflammatory Diseases

### Cancer

Cancer is a group of more than 100 diseases associated with abnormal division and cell growth with characteristic spreading in the body. Many cancers are in the form of tumors, e.g. breast cancer, lung cancer, colon cancer, ovarian cancer, renal cancer, prostate cancer, head and neck cancer, pancreas cancer, bone cancer, and thyroid cancer. Cancers associated with blood and lymphold tissues may be referred to as liquid tumors, e.g. leukemia, lymphoma and myeloma. Cancer is caused by failure of tissue growth regulation. Genes associated with cancer include oncogenes, that promote cell growth and reproduction, and tumor suppressor genes, that inhibit cell division. Oncogenes include, but are not limited to, growth factors, receptor and cytoplasmic tyrosine kinases, transcription factors, serine/threonine kinases and regulatory GTPases. Tumor protein p53 is the most common tumor suppressor protein found in more than half of cancer types. Susceptibility to cancer is involved with environmental factors, as well as genetic. Though progress with prevention, diagnosis and treatment of cancer has been tremendous, cancer remains a severe and life-threatening disease. According to American Cancer Society, an estimated 1.6 cancers are diagnosed annually in the US, leading to more than a half a million deaths.

In one embodiment, the cancer may be Leptomeningial metastases and/or glioblastoma.

Therapies associated with cancer treatment include surgery, chemotherapy, radiation and antibody therapies. Antibodies for treatment and/or prevention of cancers have been on the market for nearly two decades, and are considered one of the most important strategies for treatment of e.g. hematological malignancies and solid tumors. A number of cancer-associated antigens have been identified for treatment of cancers. Antibodies targeting such antigens may be used to diagnose, prevent and/or treat the associated cancers (see, e.g. Scott et al, 2012, Nature Reviews Cancer 12, 278-287, and references therein).

Some solid cancer tumors are associated with expressed glycoproteins antigens. Such antigens include, but are not limited to, EPCAM (Epithelial cell adhesion molecule), CEA (Carcinoembryonic antigen), gpA33 (Glycoprotein A33 (Transmembrane)), mucins, TAG-72 (Tumor-associated glycoprotein 72), CAIX (Carbonic anhydrase IX), PSMA (Prostate-specific membrane antigen), and FBP (Folate-binding protein). Antibodies targeting the expressed glycoproteins may be used to treat associated tumors. Such solid tumors include, but are not limited to, breast, colon cancer, lung, colorectal, ovarian, renal cell, and/or prostate tumors.

Some solid cancer tumors are associated with growth factor and differentiation signaling associated antigens. Such antigens include, but are not limited to, EGFR/ERBB1/HER1 (epidermal growth factor receptor 1), ERBB2 (epidermal growth factor receptor 2), ERBB3 (epidermal growth factor receptor 3), MET (Tyrosine-Protein Kinase Met), IGF1R (insulin-like growth factor 1 receptor), EPHA3 (EPH Receptor A3), TRAILR1, (Death receptor 4), and (Receptor activator of nuclear factor kappa-B ligand), Cancers that may be treated with antibodies targeting the growth factor and differentiation signaling include, but are not limited to, breast, colon, lung, ovarian, prostate, head and neck, pancreas, thyroid, kidney, and colon tumors, melanoma, glioma, bone metastases, and hematological malignancies.

Some cancer tumors are associated with antigens of stromal and extracellular matrix, Such antigens include, but are not limited to, tenascin and FAP (Fibroblast Activation Protein, Alpha). Cancers that may be treated with antibodies targeting the stromal and extracellular matrix antibodies include, but are not limited to, breast, prostate, colon, lung, pancreas and head and neck tumors and glioma.

Some cancer tumors are associated with such as Lewis-Y Le(y) antigen. Le(y) antigen has been found expressed on a number of cancers, such as, but not limited to, ovarian, breast, colon, lung and prostate cancer. Antibodies targeting Le(y) antigen may be used to treat the associated cancers.

Some cancer tumors are associated with glycolipid antigens. Such antigens include, but are not limited to, gangliosides, such as GD2, GD3, and GM2 (monosialotetrahexosylganglioside 2). Cancers that may be treated with antibodies targeting the glycolipid antigens include, but are not limited to, epithelial tumors (e.g. breast, colon and lung tumors) and neuroectodermal tumors (tumors of the central and peripheral nervous system).

The vasculature of solid tumors is abnormal, compared to normal vasculature. Antigens supporting the formation of abnormal microvasculature and progress of cancer include, but are not limited to, VEGF (Vascular endothelial growth factor), VEGFR (vascular endothelial growth factor receptor), integrin αVβ3 and integrin α5β1. Antibodies targeting such antigens may be used to treat a number of solid tumors such as, but not limited to, lung, breast, renal, brain, eye, colorectal, melanoma, ovarian, and/or other tumors, by preventing the formation of abnormal vasculature.

Hematopoietic and lymphoid malignancies are cancers affecting the blood, bone marrow, lymph and lymphatic system. Such cancers include e.g. leukemias (acute and chronic lymphoblastic leukemia, acute and chronic myelogenous leukemia), lymphomas (Hodgkin's lymphoma, Non-Hodgkin's lymphoma) and myelomas. Tumors of the hematopoietic and lymphoid tissues are closely related to immune systems. Hematological tumors may be caused by chromosomal abnormalities derived from the myeloid and lymphoid cell lines. The lymphoid cell line produces T and B cells, whereas myeloid cell line produces granulocytes, erythrocytes, thrombocytes, macrophages and mast cells. T and B cell associated hematopoietic differentiation antigens are glycoproteins that are usually from cluster of differentiation (CD) group, such as, but not limited to, CD20, CD30, CD33 and CD52. Antibodies targeting such antigens may be used for prevention and/or treatment of hematopoietic and lymphoid cancers.

In some embodiments, methods of the present disclosure may be used to treat subjects suffering from a cancer. In some cases, methods of the present disclosure may be used to treat subjects suspected of developing a cancer.

In some embodiments, methods of the present disclosure may be used for immuno-oncology (I-O) applications. AAV particles or pharmaceutical compositions of the present disclosure may be used to develop an immunotherapy or as an immunotherapy in an I-O treatment of a subject suffering from cancer. Non-limiting examples of I-O applications include active, passive or hybrid immunotherapies, checkpoint blockade, adoptive cell transfer (ACT), cancer vaccines, CAR or CAR-T therapies, dendritic cell therapy, stem cell therapies, natural killer (NK) cell-based therapies, and interferon or interleukin based methods.

AAV particles and methods of using the AAV particles described in the present disclosure may be used to prevent, manage and/or treat cancer. As a non-limiting example, the AAV particles of the present disclosure comprise a nucleic acid sequence encoding at least one of the sequences described in Table 4 (SEQ ID NO: 2142-7346).

### Immune and Autoimmune Diseases

The human immune system is a complex mechanism for identifying and removing harmful environmental agents and repairing the harm and damage caused by them. In general, immune system identifies the body's own substances from substances acquired, in other words, the self from the non-self. The immune system can be subdivided into innate and adaptive systems. The innate system is always present and includes macrophages, dendritic cells, myeloid cells (neutrophils, mast cells, basophils, eosinophils) NK cells, complement factors and cytokines. The adaptive system responds to infectious agents, and includes T and B lymphocytes, antibodies and cytokines. Activation of T and B cells in the absence of an infectious agents leads to autoimmune diseases (see, e.g. Mackay et al., 2001, N Engl J Med, Vol. 345, No. 5, and references therein). Autoimmune diseases may affect several tissues and biological functions, e.g. joints, skin, blood vessels, muscles, organs, intestine etc. Autoimmune diseases arise from an overactive and misguided immune response to the body's natural tissues and species. Autoimmune diseases and conditions include, but are not limited to, rheumatoid arthritis, diabetes type 1, systemic lupus erythematosus, celiac sprue, psoriasis, Graves' disease, and Lyme disease. Autoimmune diseases may be caused by infections, drugs, environmental irritants, toxins, and/or genetic factors. Autoimmune diseases affect up to 50 million individuals in the US. Two most common autoimmune diseases are rheumatoid arthritis and autoimmune thyroiditis, together affecting approximately 5% of population in Western countries.

Though medical therapies for autoimmune diseases exits, the diseases may still significantly lower the quality of life, or even be fatal. There remains a need for medical therapies affecting the pathophysiology of autoimmune diseases. Autoimmune disease pathophysiology is associated with several factors and may be prevented and/or treated by antibodies targeting associated proteins. Such targets include, but are not limited to, infectious agents; environmental triggers (e.g. gliadin); targets affecting cytokine production or signaling (e.g. TNFa (tumor necrosis factor alpha), IL-1 (interleukin 1-receptor), IL-2 (interleukin-2), IL-2R (interleukin-2 receptor), IL-7 (interleukin-7), IL-10 (interleukin-10), IL-10R (interleukin-10 receptor), interferon-y, STAT-3 (Signal transducer and activator of transcription 3), STAT-4 (Signal transducer and activator of transcription 4), TGF beta (transforming growth factor beta), T cell trans TGF beta); T cell regulators (e.g. CTLA4 (Cytotoxic T-Lymphocyte-Associated Protein 4)); complement components (e.g. C1 and C4); TNFa (tumor necrosis factor alpha) and TNFb (tumor necrosis factor beta); T cell regulators (e.g. CD1); epitopes of Band T cells; and/or other targets, such as those associated with B and C cells. (see, e.g. Mackay et al., 2001, N Engl J Med, Vol. 345, No. 5, and references therein).

In some embodiments, methods of the present disclosure may be used to treat subjects suffering from an autoimmune disease. In some cases, methods of the present disclosure may be used to treat subjects suspected of developing an autoimmune disease.

In some embodiments, AAV particles and/or the methods of the present disclosure may be used to treat autoimmune diseases such as systemic sclerosis (SSc). In one embodiment, the payload region may encode antibodies or fragments thereof that target anti-neutrophil cytoplasmic antibodies (ANCA). In one embodiment, the AAV particles may be used to treat ANCA-associated vasculitis.

AAV particles and methods of using the AAV particles described in the present disclosure may be used to prevent, manage and/or treat immune system and autoimmune disease. As a non-limiting example, the AAV particles of the present disclosure comprise a nucleic acid sequence encoding at least one of the sequences described in Table 4 (SEQ ID NO: 2142-7346).

### Inflammatory Disorders

Inflammation is a natural response of the body to an irritation e.g. by infection, damaged cells or other harmful agents. The purpose of the inflammation is to remove the cause of irritation and necrotic cells and damaged tissues and to initiate cell and tissue repair. Inflammation has a role in most diseases. Inflammatory disorders are abnormalities in the body's ability to regulate inflammation. Over 100 disorders associated with high levels of inflammation have been identified, including, but not limited to, Alzheimer's disease, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, and ulcerative colitis. Many inflammatory disorders are severe, and even life-threatening. Antibodies targeting proteins associated with inflammation may be used to prevent, manage or treat inflammatory disorders as well as inflammation associated diseases.

A large number of proteins are associated in inflammation, including, but not limited to, TNF (anti-tumor necrosis factor), IL-1R (Interleukin-1 receptor), IL-6R (Interleukin-6 receptor), Alpha integrin subunit, CTLA4 (Cytotoxic T-Lymphocyte. Associated Protein 4), and CD20 (see, e.g. Kotsovilis and Andreakos, 2014, Michael Steinitz (ed.), Human Monoclonal Antibodies: Methods and Protocols, Methods in Molecular Biology, vol. 1060, and references therein). For example, adalimumab (developed by Abbot Laboratories) is a TNF-targeting antibody for rheumatoid arthritis and other arthritis, psoriasis, and Crohn's disease and Natalizumab (developed by Biogen Idec) is an antibody targeting alpha 4-integrin for treatment of Crohn's disease. Additionally, plethora of cytokines, chemokines, adhesion and co-stimulatory molecules, receptors, as well as diverse cell types, may have a role in inflammatory diseases.

In some embodiments, methods of the present disclosure may be used to treat subjects suffering from an inflammatory disease. In some cases, methods of the present disclosure may be used to treat subjects suspected of developing an inflammatory disease.

AAV particles and methods of using the AAV particles described in the present disclosure may be used to prevent, manage and/or treat inflammatory disorders and inflammation. As a non-limiting example, the AAV particles of the present disclosure comprise a nucleic acid sequence encoding at least one of the sequences described in Table 4 (SEQ ID NO: 2142-7346).

### Blood and Blood Vessel Diseases

Systemic diseases are a category of conditions affecting the whole body, or many tissues and organs of the body. Systemic conditions associated with the blood, blood vessels, and cardiovascular system, include, but are not limited to, heart failure, acute coronary syndrome, atherosclerosis, hypertension, lung disease, cardiomyopathy, hyperlipidemia, hypercholesterolemia, hypertriglyceridemia, blood clotting, cardiopulmonary bypass, myocardial infection, platelet aggregation and hemolytic diseases. In general, such conditions affect quality of life and may ultimately be life-threatening. Cardiovascular diseases, referring to heart- and blood vessel-related conditions, are the leading cause of death worldwide. There remains a need for therapies affecting the pathophysiology of systemic heart, blood and blood circulation diseases. Antibodies for treating such conditions have been developed, targeting proteins such as, but not limited to, selectin P, integrin αIIbβ3, GPIIb/IIIa, RHD (Rh blood group, D antigen), PCSK9 (proprotein convertase subtilisin/kexin type 9), oxLDL (Oxidized low-density lipoprotein), CD20 (B-lymphocyte antigen), ANGPTL3 (Angiopoietin-Like 3), F9 (human factor 9), F10 (human factor 10), TFPI (Tissue Factor Pathway Inhibitor (Lipoprotein-Associated Coagulation Inhibitor)), CD41 (Integrin, Alpha 2b (Platelet Glycoprotein IIb Of IIb/IIIa Complex, Antigen CD41).

In some embodiments, methods of the present disclosure may be used to treat subjects suffering from blood-, blood circulation- and heart-related systemic diseases. In some cases, methods of the present disclosure may be used to treat subjects suspected of developing systemic blood-, blood circulation- and heart-related systemic diseases.

Stroke is a medical emergency characterized either by a burst, in particular, of a blood vessel in the brain, referred to as hemorrhagic stroke, or by an interruption of blood supply in the brain, referred to as ischemic stroke. Stroke triggers inflammation and causes brain cell death as oxygen and nutrient supplies are suddenly restricted. Typical symptoms include numbness or weakness, especially on one side of the body, confusion, trouble speaking and understanding speech, vision problems, dizziness and loss of balance. Typically, patients recovering from stroke have permanent disabilities, such as those affecting, e.g., movement, speech, coordination, vision and balance. Medical conditions, e.g., diabetes, high blood pressure, high cholesterol, and obesity, as well as, cigarette smoking and poor nutrition, increase susceptibility to a stroke. According to CDC, stroke affects about 800,000 people in the US annually and is the fifth most common cause of death.

Typical recovery from a stroke is slow and often impartial. The inability of the central nervous system (CNS) to repair after injury has been partly attributed to inhibitory proteins associated with the CNS. For example, myelin-associated proteins, such as, but not limited to, myelin associated glycoprotein (MAG), myelin associated inhibitor (MAI), and their receptors, proteoglycans, versican V2, oligodendrocyte myelin glycoprotein (Omgp), and neurite outgrowth inhibitor (Nogo) have been identified to inhibit neurite outgrowth (see, e.g. Yu et al., 2013, Transl Stroke Res, 4(5):477-83, and references therein). Cell death in ischemic stroke has been associated with excessive activation of glutamate receptors such as, but not limited to, N-methyl-D-aspartic acid (NMDA) receptors and DL-α-amino-3-hydroxy-5-methyl-4-isoxazole propionic acid (AMPA). Inflammatory signaling triggered after stroke has been associated with adhesion molecules of the endothelial cells, such as, but not limited to, those represented in the selectin family, intercellular adhesion molecule-1 (ICAM-1, also known as CD54), and 02-integrins.

Therapies to prevent stroke are typically focused on treatment of underlying medical conditions. Acute treatment following ischemic stroke involves dissolution of the blood clot, e.g., by antiplatelet agents, anticoagulants and thrombolytics. Treatment of hemorrhagic stroke involves quenching of bleeding. There is presently no effective preventative therapy for stroke. There remains a need for therapy addressing the underlying pathophysiology of stroke. Antibodies targeting stroke-associated proteins have been developed. For example, Refanezumab is a monoclonal antibody targeting myelin-associated glycoprotein, MAG, for improvement and recovery of motor function after stroke.

In some embodiments, methods of the present disclosure may be used to prevent a stroke, or treat individuals recovering from a stroke.

AAV particles and methods of using the AAV particles described in the present disclosure may be used to prevent, manage and/or treat blood- and blood vessel-related diseases, including those related to the cardiovascular system, and stroke. As a non-limiting example, the AAV particles of the present disclosure comprise a nucleic acid sequence encoding at least one of the sequences described in Table 5 (SEQ ID NO: 7347-7517).

### Respiratory Diseases

Respiratory diseases are characterized by the dysfunction of any organ, tissue and/or structure that allows for breathing, air distribution and/or for the exchange of gases, e.g., oxygen and carbon dioxide, between the air and blood, e.g., nasal cavity and sinuses, larynx, pharynx, trachea, bronchi, and lungs, including alveoli and alveolar ducts. Individuals with respiratory diseases may have impaired breathing, which is often associated with reduced quality of life and, ultimately, can be fatal. Respiratory diseases that are specifically associated with the lungs may be termed pulmonary diseases.

Several diseases, disorders, and conditions are associated with respiratory and/or pulmonary health. For example, such diseases, disorders, and conditions include, but are not limited to, Bronchitis, Acute Respiratory Distress Syndrome (ARDS), Alpha-1 Antitrypsin Deficiency, Asbestosis, Asthma, Bronchiectasis, Bronchiolitis, Bronchiolitis Obliterans with Organizing Pneumonia (BOOP), Bronchopulmonary Dysplasia, Byssinosis, Chronic Bronchitis, Chronic Cough, Chronic Obstructive Pulmonary Disease (COPD), Chronic Thromboembolic Pulmonary Hypertension (CTEPH), Coccidioidomycosis, Cough, Cryptogenic Organizing Pneumonia (COP), Cystic Fibrosis (CF), Deep Vein Thrombosis (DVT)/Blood Clots, Emphysema, Hantavirus Pulmonary Syndrome (HPS), Histoplasmosis, Human Metapneumovirus (hMPV), Hypersensitivity Pneumonitis, Idiopathic Pulmonary Fibrosis (IPF), Influenza (Flu), Interstitial Lung Disease (ILD), Lung Cancer, Lymphangioleiomyomatosis (LAM), Mesothelioma, Middle Eastern Respiratory Syndrome (MERS), Nontuberculosis Mycobacteria (NTM), Pertussis, Pneumoconiosis, Pneumonia, Primary Ciliary Dyskinesia (PCD), Pulmonary Arterial Hypertension (PAH), Pulmonary Fibrosis (PF), Pulmonary Hypertension, Respiratory Syncytial Virus (RSV), Sarcoidosis, Severe Acute Respiratory Syndrome (SARS), Shortness of Breath, Silicosis, Sleep Apnea (OSA), Sudden Infant Death Syndrome (SIDS), Tuberculosis (TB).

There are medical therapies for the management of respiratory and/or pulmonary diseases such as, but not limited to, aclidinium (Tudorza), arformoterol (Brovana), formoterol (Foradil, Perforomist), glycopyrrolate (Seebri Neohaler), indacaterol (Arcapta), Kalydeco (ivacaftor), olodaterol (Striverdi Respimat), Roflumilast (Daliresp), salmeterol (Serevent), tiotropium (Spiriva), glycopyrrolate/formoterol (Bevespi Aerosphere), glycopyrrolate/indacaterol (Utibron Neohaler), tiotropium/olodaterol (Stiolto Respimat), umeclidinium/vilanterol (Anoro Ellipta), budesonide/formoterol (Symbicort), fluticasone/salmeterol (Advair), fluticasone/vilanterol (Breo Ellipta). Several therapies for the management of respiratory and/or pulmonary diseases may include, but are not limited to, one or more of corticosteroids, cystic fibrosis transmembrane conductance regulator (CFTR) modulators, short- and long-acting bronchodilators, digestive enzymes, Methylxanthines, mucolytics, antibiotics, and vitamins. However, the present medical therapies have side effects and/or require frequent administration. There remains a need for efficient and long-lasting medical therapy affecting the pathophysiology of respiratory- and/or pulmonary-associated disease.

In some embodiments, methods of the present disclosure may be used to treat subjects suffering from respiratory- and/or pulmonary-associated disease, and/or other conditions associated with breathing, air distribution, and/or the exchange of gases between the air and the blood. In some cases, methods of the present disclosure may be used to treat subjects suspected of developing respiratory- and/or pulmonary-associated disease, and/or other conditions associated with breathing, air distribution, and/or the exchange of gases between the air and the blood.

AAV particles and methods of using the AAV particles described in the present disclosure may be used to prevent, manage and/or treat respiratory- and/or pulmonary-associated disease, and/or other conditions associated with breathing, air distribution and/or the exchange of gases between the air and the blood. As a non-limiting example, the AAV particles of the present disclosure comprise a nucleic acid sequence encoding at least one of the sequences described herein.

As a non-limiting example, the AAV particles of the present disclosure comprise a nucleic acid sequence encoding at least one of the sequences described in Table 6 (SEQ ID NO: 7518-7574).

### Muscle Diseases

Several diseases, disorders and condition are associated with muscle weakness, which refers to reduced muscle mass, muscle strength and muscle function. For example, such disorders include myopathies, which are neuromuscular disorders characterized by muscle weakness due to dysfunction of muscle fiber. Myopathies include, but are not limited to, congenital myopathies, muscular dystrophies, mitochondrial myopathies, glycogen storage diseases of muscle, myoglobinurias, dermatomyositis, myositis ossificans, familial periodic paralysis, polymyositis, inclusion body myositis, and related myopathies, neuromyotonia, stiff-man syndrome, common muscle cramps and stiffness, and tetany. Muscle weakness may also be caused by ageing, diabetes, obesity, chronic pain, peripheral vascular disease, chronic lung diseases, heart diseases, cancers, anemia, arthritis, chronic renal failure and renal diseases, chronic obstructive pulmonary disease, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's syndrome, acute respiratory distress syndrome, steroid induced muscle wasting, myositis, scoliosis, or infections e.g. influenza, Epstein-Barr virus infection, HIV/AIDS, Lyme disease, and hepatitis C infection. Muscle weakness may occur after surgery, burn trauma, medical treatment, or trauma through an injury. Severity of muscle weakness varies. In many cases the muscle dysfunction, including weakness, reduces quality of life significantly, or may be life-threatening.

A regulator protein associated with muscles is myostatin (MSTN), also known as growth and differentiation factor 8 (GDF8). Myostatin is a protein encoded by the MSTN gene, released in the myocytes. Myostatin and myostatin receptors (e.g. ACVR2A and ACVR2B), have a role in suppressing the growth and development of muscle tissue in the body.

Treatment of muscle weakness depends on the underlying disease or condition, and may include, e.g., drug therapy, good nutrition, physiotherapy, mechanical support for weakened muscles such as those that may arise following surgery. However, efficient therapy to treat a combination of loss of muscle mass, muscle strength and muscle function are needed. Antibodies targeting myostatin may be used in the treatment and prophylaxis of diseases associated with such conditions. For example, bimagrumab (developed by Novartis) is a monoclonal antibody targeting ACVR2B myostatin receptor, and used for therapy of musculoskeletal diseases and domagrozumab (developed by Pfizer) is an antibody targeting myostatin, and used for therapy of muscle degeneration and muscle weakness.

In some embodiments, methods of the present disclosure may be used to treat subjects suffering from loss of muscle mass, muscle strength and/or muscle function. In some cases, methods of the present disclosure may be used to treat subjects suspected of developing such conditions.

AAV particles and methods of using the AAV particles described in the present disclosure may be used to prevent, manage and/or treat diseases associated with muscle mass, muscle strength and/or muscle function. As a non-limiting example, the AAV particles of the present disclosure comprise a nucleic acid sequence encoding at least one of the sequences described herein.

### Bone Diseases

Osteoporosis is a disease characterized by a reduced bone mineral density, and disrupted bone microarchitecture. Individuals with osteoporosis have a high susceptibility to bone fractures. Osteoporosis causes disability, especially in the elderly, and may be fatal.

There are medical therapies for management of the osteoporosis, and other conditions associated with reduced bone density, such as calcitonin, bisphosphonates, estrogen replacement and selective estrogen modulators for prevention of bone loss, and anabolic agents to increase bone mass and bone mineral density. However, the present medical therapies have side effects and/or require frequent administration. There remains a need for efficient and long-lasting medical therapy affecting the pathophysiology of osteoporosis and other conditions associated with reduced bone density, such as antibody therapies. Antibodies for treatment of osteoporosis are on the market, e.g. blosozumab (developed by Eli Lilly and Co.) targeting sclerostin (SOST) for increasing bone density, and denosumab (developed by Amgen) targeting TNFSF11 (Tumor Necrosis Factor (Ligand) Superfamily, Member 11) for treatment of bone loss.

In some embodiments, methods of the present disclosure may be used to treat subjects suffering from osteoporosis and/or other conditions associated with reduced bone density. In some cases, methods of the present disclosure may be used to treat subjects suspected of developing osteoporosis and/or other conditions associated with reduced bone density.

AAV particles and methods of using the AAV particles described in the present disclosure may be used to prevent, manage and/or treat systemic diseases of the blood, heart and/or bone. As a non-limiting example, the AAV particles of the present disclosure comprise a nucleic acid sequence encoding at least one of the sequences described herein.

### Endocrine and Metabolic Diseases

Endocrine diseases are characterized by the dysfunction of a network of glands and organs that produce and release specific hormones to regulate and control critical body processes such as growth and development, sexual function, reproduction, mood, and metabolism. Endocrine glands include the adrenal glands, ovaries, pancreatic islet cells, parathyroid, pineal gland, pituitary gland, testes, thymus, and thyroid. The thyroid gland is specifically important for the regulation of metabolism, and in turn, digestion, elimination, breathing, blood circulation and homeostatic mechanisms, from cellular to organismal levels. Metabolic diseases and disorders occupy a substantial proportion of all endocrine diseases and disorders, representing their own subclass. Such diseases, however, more broadly involve impaired chemical processing of e.g., amino acids, carbohydrates, lipids to acquire or generate energy. Generally, endocrine disorders involve either hormone over or under production/secretion, in other words, hyper or hypo function, respectively. Individuals with endocrine diseases, including those that are metabolic in nature, may experience diverse symptoms including abnormal heart rate/rhythms, anxiety, weight gain/loss, excess body fat or obesity, weakness, abnormal blood glucose, sleep problems, etc.

Several diseases, disorders, and conditions are associated with endocrine and/or metabolic health. For example, such diseases, disorders, and conditions include, but are not limited to, Acromegaly, Addison's Disease, Adrenal Cancer, Adrenal Disorders, Anaplastic Thyroid Cancer, Cushing's Syndrome, De Quervain's Thyroiditis, Diabetes, Follicular Thyroid Cancer, Gestational Diabetes, Goiters, Graves'Disease, Growth Disorders, Growth Hormone Deficiency, Hashimoto's Thyroiditis, Heart Disease, Hurthle Cell Thyroid Cancer, Hyperglycemia, Hyperparathyroidism, Hyperthyroidism, Hypoglycemia, Hypoparathyroidism, Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Reproduction, Silent Thyroiditis, Thyroid Cancer, Thyroid Diseases, Thyroid Nodules, Thyroiditis, Turner Syndrome, Type 1 Diabetes, Type 2 Diabetes.

There are medical therapies for the management of endocrine and/or metabolic diseases such as, but not limited to, abaloparatide, acarbose, Acthar Gel, Actonel, Actonel with Calcium, ACTOplus Met, ACTOplus Met XR, Adagen, ADH, Adlyxin, Afrezza, agalsidase alfa, agalsidase beta, AHydrocort, albiglutide, Aldurazyme, alendronate, alglucerase, alglucosidase alfa, alogliptin, alogliptin/metformin, alogliptin/pioglitazone, Alphosyl, Amaryl, A-Methapred, Anadrol-50, Androderm, AndroGel, Android, Androxy, Apidra, Apidra Solostar, Aquacort, Aredia, Armour Thyroid, asfotase alfa, Atelvia, Avandamet, Aveed, Axiron, Basaglar, Baycadron, becaplermin, beta glucuronidase, recombinant, betaine, Betaject, betamethasone, Betamethasone IM/PO, Binosto, Boniva, Brineura, bromocriptine, Bydureon, Bydureon BCise, Byetta, calcifediol, Calcijex, calcitonin salmon, calcitriol, canagliflozin/metformin, carnitine, Carnitor, Celestone, Celestone Soluspan, Cerdelga, Ceredase, Cerezyme, cerliponase alfa, chlorpropamide, cinacalcet, conivaptan, Contributor Login, Cortef, Cortenema, corticotropin, cortisone, Covaryx, Cycloset, Cystadane, Cystagon, cysteamine, Cytomel, D50W, danazol, dapagliflozin/metformin, dapagliflozin/saxagliptin, DDAVP, Decadron, deflazacort, Delatestryl, Deltasone, denosumab, DepoMedrol, Depo-Testosterone, desmopressin, dexamethasone, Dexamethasone Intensol, Dexasone, dextrose, DGlucose, Diabeta, Diabinese, diazoxide, dichlorphenamide, Didronel, doxercalciferol, dulaglutide, eculizumab, Elaprase, Elelyso, eliglustat, elosulfase alfa, Emflaza, empagliflozin/linagliptin, empagliflozin/metformin, ertugliflozin/metformin, ertugliflozin/sitagliptin, Estratest, Estratest H.S., estrogens esterified/methyltestosterone, etelcalcetide, etidronate, Evenity, exenatide injectable solution, exenatide injectable suspension, exenatide subdermal implant, Fabrazyme, Flasp, FloPred, Florinef, Florinef Acetate, fludrocortisone, fluoxymesterone, Fortamet, Forteo, Fortesta, Fosamax, Fosamax Plus D, galsulfase, Genotropin, Genotropin Miniquick, Genotropin Pen 12, glimepiride, glipizide, Glucagen, GlucaGen HypoKit, glucagon, Glucagon Emergency Kit, Glucophage, Glucophage XR, glucose, Glucotrol, Glucotrol XL, Glumetza, glyburide, Glynase, Glynase PresTab, Glyset, Glyxambi, Halotestin, Hectorol, Hicon, HP Acthar Gel, Humalog, Humalog Junior KwikPen, Humalog Kwikpen, Humalog Mix 50/50, Humalog Mix 50/50 Kwikpen, Humalog Mix 75/25, Humalog Mix 75/25 Kwikpen, human parathyroid hormone, recombinant, Humatrope, HumatroPen, Humulin 70/30, Humulin N, Humulin R, Humulin R U-500, hydrocortisone, ibandronate, idursulfase, imiglucerase, Increlex, insulin aspart, insulin aspart protamine/insulin aspart, insulin degludec, Insulin degludec/insulin aspart, insulin detemir, insulin glargine, insulin glargine/lixisenatide, insulin glulisine, insulin inhaled, insulin isophane human/insulin regular human, insulin lispro, insulin lispro protamine/insulin lispro, insulin NPH, insulin regular human, Invokamet, lnvokamet-XR, losat, Janumet, Janumet XR, Januvia, Jentadueto, Jentadueto XR, Juvisync, Kanuma, Kazano, Kenalog-10, Kenalog-40, Keveyis, Kombiglyze XR, Korlym, L Thyroxine, lanreotide, Lantus, Lantus SoloStar, laronidase, Levemir, Levemir FlexTouch, Levo T, levocaritine, Levothyroid, levothyroxine, Levothyroxine T4, Levoxine, Levoxyl, linagliptin, linagliptin/metformin, liothyronine, Liothyronine T3, liotrix, liraglutide, liraglutide/insulin degludec, lixisenatide, Lumizyme, macimorelin, Macrilen, mecasermin, Medrol, Medrol Dosepak, metformin, metformin/pioglitazone, metformin/repaglinide, metformin/rosiglitazone, metformin/sitagliptin, methimazole, Methitest, methylprednisolone, methyltestosterone, metreleptin, Miacalcin, Mifeprex, mifepristone, miglitol, Millipred, Millipred DP, Minirin, Minodiab, mometasone sinus implant, Myalept, Myozyme, Naglazyme, Natesto, Natpara, Nature-Throid, Nesina, nitisinone, Nityr, Noctiva, Norditropin FlexPro, Norditropin NordiFlex, Northyx, Novolin 70/30, Novolin N, Novolin R, NovoLog, NovoLog FlexPen, NovoLog FlexTouch, NovoLog Mix 50/50, NovoLog Mix 70/30, NovoLog Mix 70/30 FlexPen, NovoPen Echo, Nutropin, Nutropin AQ, Nutropin AQ NuSpin 10, Nutropin AQ NuSpin 20, Nutropin AQ NuSpin 5, Nutropin AQ Pen 10, Nutropin AQ Pen 20, octreotide, Omnitrope, Onglyza, Orapred, Orapred ODT, Orfadin, Orinase, Oseni, Oxandrin, oxandrolone, Oxymetholone, pamidronate, parathyroid hormone, paricalcitol, Parlodel, Parsabiv, pasireotide, Pediapred, pegademase, pegvaliase, pegvisomant, pegylated phenylalanine ammonia lyase (PAL), Pima Syrup, potassium iodide, pramlintide, PrandiMet, Precose, prednisolone, prednisone, Prednisone Intensol, Prelone Syrup, Procysbi, Proglycem, Prolia, Propel, Propel Contour, Propel Mini, propylthiouracil, PropylThyracil, PTU, Qtern, Rayaldee, Rayos, Reclast, recombinant human tripeptidyl peptidase 1 (rhtpp1), Regranex Gel, Replagal, rhGUS, Riomet, risedronate, Rocaltrol, romosozumab, ru486, Ryzodeg, sacrosidase, Salzen, Samsca, Sandostatin, Sandostatin LAR, saxagliptin, saxagliptin/metformin, Saxenda, sebelipase alfa, semaglutide, Sensipar, Serostim, Signifor, Signifor LAR, sitagliptin, sitagliptin/simvastatin, Skelid, sodium iodide I-131, Soliqua 100/33, Soliris, SoluCortef, SoluMedrol, Solurex, somatropin, Somatuline Depot, Somavert, SSKI, Sterapred, Sterapred DS, Stimate, Strensiq, Striant, Sucraid, Symlin, SymlinPen 120, SymlinPen 60, Synjardy, Synjardy XR, Synthroid, Syprine, taliglucerase alfa, Tanzeum, Tapazole, teriparatide, Testim, Testopel, testosterone, testosterone buccal system, testosterone intranasal, testosterone topical, Testred, thyroid desiccated, Thyroid Hormone, Thyrolar, ThyroSafe, ThyroShield, tiludronate, Tirosint, Tirosint-SOL, tolazamide, tolbutamide, Tolinase, tolvaptan, Toujeo, Tradjenta, Tresiba, triamcinolone, Trientine, Triostat, Trulicity, Tymlos, Unithroid, uridine triacetate, Vaprisol, vasopressin, Vasostrict, velaglucerase alfa, Veripred 20, Victoza, Vimizim, Vistogard, Vogelxo, VPRIV, Xgeva, Xigduo XR, Xultophy, Xuriden, Zempla, Zilretta, zoledronic acid, Zomacton, Zometa, and Zorbtive. However, the present medical therapies have side effects and/or require frequent administration. There remains a need for efficient and long-lasting medical therapy affecting the pathophysiology of endocrine- and/or metabolism-associated disease.

In some embodiments, methods of the present disclosure may be used to treat subjects suffering from of endocrine- and/or metabolism-associated disease. In some cases, methods of the present disclosure may be used to treat subjects suspected of developing of endocrine- and/or metabolism-associated disease.

AAV particles and methods of using the AAV particles described in the present disclosure may be used to prevent, manage and/or treat endocrine- and/or metabolism-associated disease. As a non-limiting example, the AAV particles of the present disclosure comprise a nucleic acid sequence encoding at least one of the sequences described herein.

### Nervous System Diseases

### Alzheimer's Disease

Alzheimer's Disease (AD) is a debilitating neurodegenerative disease and the most common form of dementia affecting the memory, thinking and behavior. A typical early symptom is difficulty remembering newly learned information. As the disease advances, symptoms include disorientation, changes in sleep, changes in mood and behavior, confusion, unfound suspicions and eventually difficulty to speak, swallow and walk. AD currently afflicts more than 35 million people worldwide, with that number expected to double in coming decades.

As of today, no cure or prevention therapy for AD has been identified. Drug therapy to treat memory loss, behavioral changes and sleep changes, and to slow down the progression of AD are available. However, these symptomatic treatments do not address the underlying pathophysiology.

The AD brain is characterized by dual aggregates, the extracellular β-amyloid plaques and the intracellular neurofibrillary tangles (NFT) of misfolded, hyperphosphorylated microtubule associated tau protein. β-amyloid plaques may lead to pathological cascades that are associated with a number of proteins, such as, but not limited to, APP (amyloid beta (A4) precursor protein), A beta (amyloid beta), BACE (Beta-secretases), and APOE (apolipoprotein E). Historically, it has been thought that amyloid pathology precedes the appearance of NFT, and therefore, that tau pathology in the form of aggregates is symbolic of impending cell death (Selkoe, D. J., 2001, Physiological Reviews, 81(2):741-66). However, clinical trials addressing amyloid pathology have largely failed thus far and advances in the field suggest that targeting tau aggregates may be advantageous and lead to improved cognitive ability.

In some embodiments, methods of the present disclosure may be used to treat subjects suffering from AD. In some cases, methods of the present disclosure may be used to treat subjects suspected of developing AD.

AAV particles and methods of using the AAV particles described in the present disclosure may be used to prevent, manage and/or treat AD. As a non-limiting example, the AAV particles of the present disclosure comprise a nucleic acid sequence encoding at least one of the sequences described in Table 7 (SEQ ID NO:7575-7966).

As a non-limiting example, the AAV particles of the present disclosure comprise a nucleic acid sequence encoding at least one of the sequences described in Table 8 (SEQ ID NO: 7697-84026).

As a non-limiting example, the AAV particles of the present disclosure comprise a nucleic acid sequence encoding at least one of the sequences described in Table 10 (SEQ ID NO: 8077-8174).

### Parkinson's Disease

Parkinson's Disease (PD) is a progressive disorder of the nervous system affecting especially the substantia nigra of the brain. PD develops because of the loss of dopamine-producing brain cells. Typical early symptoms of PD include shaking or trembling of a limb, e.g. hands, arms, legs, feet and face. Additional characteristic symptoms are stiffness of the limbs and torso, slow movement or an inability to move, impaired balance and coordination, cognitional changes, and psychiatric conditions, e.g. depression and visual hallucinations. PD has both familial and idiopathic forms and it has been suggested that PD arises from both genetic and environmental origins. PD affects more than 4 million people worldwide. In the US, approximately 60,000 cases are identified annually. Generally, PD begins at the age of 50 or older. An early-onset form of the condition begins at an age younger than 50, and juvenile-onset PD begins before age of 20.

Death of dopamine producing brain cells related to PD has been associated with aggregation, deposition and dysfunction of alpha-synuclein protein (see, e.g. Marques and Outeiro, 2012, Cell Death Dis. 3:e350, Jenner, 1989, J Neurol Neurosurg Psychiatry. Special Supplement, 22-28, and references therein). Studies have suggested that alpha-synuclein has a role in presynaptic signaling, membrane trafficking and regulation of dopamine release and transport. Alpha-synuclein aggregates, e.g. in the form of oligomers, have been suggested as the species responsible for neuronal dysfunction and death. Mutations of the alpha-synuclein gene (SNCA) have been identified in the familial forms of PD, but also environmental factors, e.g. neurotoxin affect alpha-synuclein aggregation, may contribute to disease pathogenesis. Other suggested causes of brain cell death in PD are dysfunction of proteasomal and lysosomal systems, reduced mitochondrial activity.

PD is related to other diseases associated with alpha-synuclein aggregation, referred to as “synucleinopathies.” Such diseases include, but are not limited to, Parkinson's Disease Dementia (PDD), multiple system atrophy (MSA), dementia with Lewy bodies, juvenile-onset generalized neuroaxonal dystrophy (Hallervorden-Spatz disease), pure autonomic failure (PAF), neurodegeneration with brain iron accumulation type-1 (NBIA-1) and combined Alzheimer's and Parkinson's disease.

As of today, no cure or prevention therapy for PD has yet been identified. A variety of drug therapies available provide relief to the symptoms. Non-limiting examples of symptomatic medical treatments include carbidopa and levodopa combination reducing stiffness and slow movement, and anticholinergics to reduce trembling and stiffness. Other optional therapies include. e.g. deep brain stimulation and surgery. There remains a need for therapy affecting the underlying pathophysiology of PD. For example, antibodies targeting alpha-synuclein protein, or other proteins relevant for brain cell death in PD, may be used to prevent and/or treat PD.

In some embodiments, methods of the present disclosure may be used to treat subjects suffering from PD and other synucleinopathies. In some cases, methods of the present disclosure may be used to treat subjects suspected of developing PD and other synucleinopathies.

AAV particles and methods of using the AAV particles described in the present disclosure may be used to prevent, manage and/or treat PD. As a non-limiting example, the AAV particles of the present disclosure comprise a nucleic acid sequence encoding at least one of the sequences described in Table 7 (SEQ ID NO:7755-7966).

As a non-limiting example, the AAV particles of the present disclosure may include a nucleic acid sequence encoding at least one of the sequences described in Table 9 (SEQ ID NO: 8027-8076).

Dementia with Lewy Bodies

Dementia with Lewy Bodies (DLB), also known as diffuse Lewy body disease, is a form of progressive dementia, characterized by cognitive decline, fluctuating alertness and attention, visual hallucinations and parkinsonian motor symptoms. DLB may be inherited by an autosomal dominant pattern. DLB affects more than 1 million individuals in the US. The condition typically shows symptoms at the age of 50 or older.

DLB is caused by the abnormal build-up of Lewy bodies, aggregates of the alpha-synuclein protein, in the cytoplasm of neurons in the brain areas controlling memory and motor control. The pathophysiology of these aggregates is very similar to aggregates observed in Parkinson's disease and DLB also has similarities to Alzheimer's disease. Inherited DLB has been associated with gene mutations in SNCA and SNCB genes, producing synuclein proteins.

As of today, there is no cure or prevention therapy for DLB. A variety of drug therapies available are aimed at managing the cognitive, psychiatric and motor control symptoms of the condition. Non-limiting examples of symptomatic medical treatments include e.g. acetylcholinesterase inhibitors to reduce cognitive symptoms, and levodopa to reduce stiffness and loss of movement. There remains a need for therapy affecting the underlying pathophysiology. Antibodies targeting alpha-synuclein protein may be used to prevent and/or treat DLB.

In some embodiments, methods of the present disclosure may be used to treat subjects suffering from DLB. In some cases, methods of the present disclosure may be used to treat subjects suspected of developing DLB.

AAV particles and methods of using the AAV particles described in the present disclosure may be used to prevent, manage and/or treat DLB. As a non-limiting example, the AAV particles of the present disclosure comprise a nucleic acid sequence encoding at least one of the sequences described in Table 7 (SEQ ID NO: 7755-7966) and/or Table 11 (SEQ ID NO: 13165-13518).

### Huntington's Disease

Huntington's disease (HD) is a rare, inherited disorder causing degeneration of neurons in the motor control region of the brain, as well as other areas. Typical symptoms of the disease include uncontrolled movements (chorea), abnormal postures, impaired coordination, slurred speech and difficulty of feeding and swallowing accompanied by changes in behavior, judgment and cognition. HD is caused by mutations in the gene associated with the huntingtin (HTT) protein. The mutation causes the (CAG) blocks of DNA to repeat abnormally many times. HD affects approximately 30, 000 individuals in the US.

HD is characterized by mutations of the huntingtin (HTT) protein with abnormal expansions of polyglutamine tracts, e.g. expansion of the length of glutamine residues encoded by CAG repeats. The expansion threshold for occurrence of the disease is considered to be approximately 35-40 residues. HD is also associated with beta sheet rich aggregates in striatal neurons formed by N-terminal region of HTT. The expansions and aggregates lead to gradual loss of neurons as HD progresses. Additionally, the cell death in HD is associated with death receptor 6 (DR6) which is known to induce apoptosis.

As of today, there is no therapy to cure, or prevent the progression of the disease. Drug therapies available are aimed at management of the symptoms. For example, FDA has approved tetrabenazine to be prescribed for prevention of chorea. Additionally, e.g. antipsychotic drugs may help to control delusions, hallucinations and violent outbursts. There remains a need for therapy affecting the underlying pathophysiology, such as antibody therapies targeting the HTT protein, DR6 protein, and/or other HD associated proteins.

In some embodiments, methods of the present disclosure may be used to treat subjects suffering from HD. In some cases, methods of the present disclosure may be used to treat subjects suspected of developing HD.

AAV particles and methods of using the AAV particles described in the present disclosure may be used to prevent, manage and/or treat HD. As a non-limiting example, the AAV particles of the present disclosure comprise a nucleic acid sequence encoding at least one of the sequences described in Table 7 (SEQ ID NO: 7755-7966).

### Amyotrophic Lateral Sclerosis

Amyotrophic Lateral Sclerosis (ALS), also known as Lou Gehrig's disease or classical motor neuron disease, is a rapidly progressive and fatal neurological disease. ALS is associated with cell degeneration and death of the upper and lower motor neurons, leading to enablement of muscle movement, weakening, wasting and loss of control over voluntary muscle movement. Early symptoms include muscle weakness of hands, legs and swallowing muscles, eventually progressing to inability to breathe due to diaphragm failure. According to Centers for Disease Control and Prevention (CDC), ALS affects an estimated 12, 000-15,000 individuals in the US. About 5-10% of cases are familial.

ALS, as other non-infectious neurodegenerative diseases, has been characterized by presence of misfolded proteins, including, but not limited to, tau, amyloid-beta (A beta), alpha-synuclein, HTT (huntingtin) or SOD (superoxide dismutase 1 protein), and myelin associated inhibitors and their receptors, (see, e.g., Krishnamurthy and Sigurdsson, 2011, N Biotechnol. 28(5):511-7, and Musaro, 2013, FEBS J; 280(17):4315-22, and references therein). Familial ALS has been associated with mutations of TAR DNA-binding protein 43 (TDP-43) and RNA-binding protein FUS/TLS. Some proteins have been identified to slow down progression of ALS, such as, but not limited, to growth factors, e.g. insulin-like growth factor 1 (IGF-1), glial cell line-derived growth factor, brain-derived growth factor, vascular endothelial growth factor and ciliary neurotrophic factor, or growth factors promoting muscle growth, e.g. myostatin.

As of today, there is no prevention or cure for ALS. FDA approved drug riluzole has been approved to prolong the life, but does not have an effect on symptoms. Additionally, drugs and medical devices are available to tolerate pain and attacks associated with ALS. There remains a need for therapy affecting the underlying pathophysiology.

In some embodiments, methods of the present disclosure may be used to treat subjects suffering from ALS. In some cases, methods of the present disclosure may be used to treat subjects suspected of developing ALS.

AAV particles and methods of using the AAV particles described in the present disclosure may be used to prevent, manage and/or treat MS. As a non-limiting example, the AAV particles of the present disclosure comprise a nucleic acid sequence encoding at least one of the sequences described in Table 7 (SEQ ID NO: 7755-7966).

As a non-limiting example, the AAV particles of the present disclosure comprise a nucleic acid sequence encoding at least one of the sequences described in Table 8 (SEQ ID NO: 7697-8026).

As a non-limiting example, the AAV particles of the present disclosure may include a nucleic acid sequence encoding at least one of the sequences described in Table 9 (SEQ ID NO: 8027-8076).

### Multiple Sclerosis

Multiple sclerosis is a disease of the central nervous system (CNS). The typical early symptoms occurring between the ages of 20 to 40 include blurring vision, red-green color distortion, partial blindness, extreme muscle weakness, feeling of numbness or prickling, difficulties with coordination and balance. In severe cases MS may lead to a partial or complete paralysis. MS is believed to be an autoimmune disease as the communication between the brain and other parts of the body being disrupted as the immune system causes an inflammation within the central nervous system. MS is caused by both genetic and environmental factors, e.g. viral infections. MS is the most common neurological condition of young adults globally, affecting more than 2.3 million individuals.

At present time, the pathophysiology of MS is not fully understood. The disease is associated with a complex combination related to formation of lesions in the central nervous system, inflammation and demyelination (destruction of the protective myelin surrounding the nerve fibers) in white matter and cortex, and axon destruction (see, e.g. Longbrake et al., 2013, Curr Neurol Neurosci Rep., 13(11), and references therein). A number of myelin inhibitory proteins have been characterized in association with MS, including, but not limited to, NogoA ((Neurite outgrowth inhibitor A), Nogo receptor-1 (NgR1), myelin associated glycoprotein (MAG), oligodendrocyte glycoprotein (OM-gp), LINGO-1 (Leucine rich repeat and immunoglobin-like domain-containing protein 1), and MAI (myelin associated inhibitor). MS is also affiliated with many immune response related proteins. Non-limiting examples of such proteins include e.g. B-cell and T-cell associated proteins, such as, but not limited to, leukocyte surface antigen CD52, alpha chain of the IL-2 receptor CD25, B-cell surface molecule CD20, T helper cell CD4, and/or cytokine IL2123. Alpha 4-integrin, has been associated with inflammation of CNS, as it has a role in leukocyte adhesion and migration to the inflamed CNS. Additionally, MS patients have been characterized with elevated tumor necrosis factor (TNF) levels.

As of today, there is no prevention therapy or cure for MS. Patients in need of medical therapy may be treated with e.g. synthetic form of myelin basic protein, (Copaxone, copolymer I), antiviral proteins known as interferons, or immunosuppressant drugs e.g. mitoxantore. Some drugs are aimed at treating a symptom of MS, such as dalapridine, which is aimed at improving walking of individuals with MS. Antibodies for MS have been developed. For example, natalizumab is a monoclonal antibody targeting alpha 4 integrin, (developed by Elan Pharmaceuticals and Biogen) approved by the FDA for treatment of relapsing MS under treatment guidelines to monitor patients by physicians. Other non-limiting examples for MS antibody drugs include alemtuzumab (CD52), daclizumab (CD25), rituximab (CD20), ocrelizumab (CD20), ofatumumab (CD20), (see, e.g. Longbrake et al., 2013, Curr Neurol Neurosci Rep., 13(11), and references therein). However, many current medications have serious side effects, and there remains a need for therapy affecting the underlying pathophysiology, such as improved antibody therapies.

In some embodiments, methods of the present disclosure may be used to treat subjects suffering from MS. In some cases, methods of the present disclosure may be used to treat subjects suspected of developing MS.

AAV particles and methods of using the AAV particles described in the present disclosure may be used to prevent, manage and/or treat MS. As a non-limiting example, the AAV particles of the present disclosure comprise a nucleic acid sequence encoding at least one of the sequences described in Table 7 (SEQ ID NO: 7755-7966).

### Multiple System Atrophy

Multiple system atrophy (MSA), also known as Shy-Drager Syndrome, is a progressive neurodegenerative disorder. The characteristic symptoms are associated with failure of autonomic nervous system causing dizziness, fainting, bladder control problems, and problems regulating heart rate, blood pressure and breathing, accompanied by motor control symptoms similar to Parkinson's disease, e.g. tremor, rigidity and loss of muscle coordination. The symptoms are a reflection of the loss of nerve cells in certain areas of the brain and spinal cord. The disease typically develops around ages of 50 or 60 years. MSA affects approximately 50,000 individuals in the US.

MSA belongs to the synucleinopathies and is characterized by the appearance of glial cytoplasmic inclusions (GCIs) in oligodendrocytes, which are the myelin producing support cells of the central nervous system (see, e.g. Bleasel et al. 2014, Acta Neuropathologica Communications, 2014, 2:15, and references therein). GCIs comprise insoluble proteinaceous filaments composed of the alpha-synuclein protein. Also, tau proteins have been identified in GCIs. The pathophysiology of the CGIs is not yet fully understood but alpha-synuclein and tau proteins are suggested to have a role in the development and progression of SMA.

As of today, there is no cure or prevention therapy for MSA. A variety of drug therapies available are aimed at managing the symptoms. Non-limiting examples of symptomatic medical treatments include those used for Parkinson's disease to relief symptoms related motor movement, increased salt intake and steroid hormones for increasing blood pressure. There remains a need for therapy affecting the underlying pathophysiology. Antibodies targeting tau and alpha-synuclein proteins may be used to prevent and/or treat MSA.

In some embodiments, methods of the present disclosure may be used to treat subjects suffering from MSA. In some cases, methods of the present disclosure may be used to treat subjects suspected of developing MSA.

AAV particles and methods of using the AAV particles described in the present disclosure may be used to prevent, manage and/or treat MSA. As a non-limiting example, the AAV particles of the present disclosure comprise a nucleic acid sequence encoding at least one of the sequences described in Table 7 (SEQ ID NO: 7755-7966).

### Spinal Muscular Atrophy

Spinal muscular atrophy (SMA) is a hereditary disease causing weakness and wasting of the voluntary muscles in the arms and legs of infants and children. SMA is associated with abnormalities in the protein production of the survival motor neuron gene 1 (SMN1). Lack of the protein affects degeneration and death of lower motor neurons. Typical symptoms include floppy limbs and trunk, feeble movement of the arms and legs, difficulties in swallowing and eating, and impaired breathing. SMA is the most common genetic disorder leading to death of children under 2 years of age. SMA affects one in 6,000 to 10,000 people.

As of today, there is no cure for SMA. Therapies available are aimed at management of the symptoms and prevention of additional complications. Such therapies are associated e.g. with cardiology, movement management, respiratory care and mental health. There remains a need for therapy affecting the underlying pathophysiology of SMA.

In some embodiments, methods of the present disclosure may be used to treat subjects suffering from SMA. In some cases, methods of the present disclosure may be used to treat subjects suspected of developing SMA.

AAV particles and methods of using the AAV particles described in the present disclosure may be used to prevent, manage and/or treat SMA. As a non-limiting example, the AAV particles of the present disclosure comprise a nucleic acid sequence encoding at least one of the sequences described in Table 7 (SEQ ID NO: 7755-7966).

### Neuropathies

Neuropathies are a group of diseases or conditions affecting the nerves. Typical symptoms of neuropathies include impaired movement and sensation, cramping, pain and abnormal organ functions. Neuropathies include e.g. diabetic neuropathy, cisplatin-induced neuropathy, mononeuropathy, pyridoxine-induced neuropathy, peripheral neuropathy, small fiber peripheral neuropathy, polyneuropathy and cisplatin/pyridoxine-induced neuropathy.

As of today, there is no prevention or treatment therapy specific for neuropathies on the market. Typical treatment involves with treatment of underlying diseases, e.g. diabetes, or management of the symptoms. Therefore, there remains a need for therapy affecting the underlying pathophysiology of neuropathies, such as efficient antibody therapies. Tyrosine kinases, such as Trk receptors, have a role in regulation of the nervous system, neuronal survival and signal cascades. Antibodies targeting e.g. Trk C may be used for prevention, treatment and/or management of neuropathies, as described in US Patent U.S. Pat. No. 7,615,383, the contents of which are herein incorporated by reference in their entirety.

In some embodiments, methods of the present disclosure may be used to treat subjects suffering from neuropathies. In some cases, methods of the present disclosure may be used to treat subjects suspected of developing neuropathies.

AAV particles and methods of using the AAV particles described in the present disclosure may be used to prevent, manage and/or treat neuropathies. As a non-limiting example, the AAV particles of the present disclosure comprise a nucleic acid sequence encoding at least one of the sequences described in Table 7 (SEQ ID NO: 7755-7966) and/or Table 11 (SEQ ID NO: 13165-13518).

### Psychiatric Disorders

Psychiatric disorders are characterized by behavioral or mental condition that affects individual's ordinary ability to function. Common psychiatric disorders include, but are not limited to, Tourette syndrome, bipolar disorder, schizophrenia, anxiety, depression, panic disorder, obsessive-compulsive disorder (OCD), eating disorders (e.g. anorexia, bulimia, orthorexia, obesity), substance abuse (e.g. alcohol or drug), addiction, psychosis, phobias, mood disorders, manic-depression disorder, insomnia and other sleep disorders. Psychiatric disorders may significantly affect individual's quality of life, and in severe cases lead to harmful behavior, such as suicidal or homicidal behavior. The diseases are typically managed and treated with psychotherapy, behavioral therapy, medical therapy (e.g. antipsychotic drugs), and/or other therapies. There remains a need for improved medical therapies affecting the underlying pathophysiology of psychiatric disorders, such as antibodies targeting proteins associated with such disorders.

For example, ghrelin hormone has been associated with eating disorders, including obesity and anorexia. Antibodies targeting ghrelin may be used for prevention, management and/or treatment of eating disorders, e.g. as described in US Patent application US20060233788, the contents of which are herein incorporated by reference in their entirety.

Depression has been associated with an inhibition of peripheral cytokine activity, especially TNFa (tumor necrosis factor alpha). Antibodies targeting TNF alpha may be used for prevention, management and/or treatment of depression, e.g. as described in US Patent application US20140296493, the contents of which are herein incorporated by reference in their entirety.

OCD and OCD related diseases have been associated T-cell activation. Anx-A1 (annexin A1) is a protein promoting T-cell activation, and antibodies binding Annexin-1 may be used for prevention, management and/or treatment of OCD and related diseases, e.g. as described in US Patent application US20150004164, the contents of which are herein incorporated by reference in their entirety.

In some embodiments, methods of the present disclosure may be used to treat subjects suffering from a psychiatric disorder. In some cases, methods of the present disclosure may be used to treat subjects suspected of developing a psychiatric disorder.

AAV particles and methods of using the AAV particles described in the present disclosure may be used to prevent, manage and/or treat psychiatric disorder. As a non-limiting example, the AAV particles of the present disclosure comprise a nucleic acid sequence encoding at least one of the sequences described in Table 7 (SEQ ID NO: 7755-7966).

### Migraine

Migraine is a neurological condition characterized by reoccurring attacks of severe headache, accompanied by nausea, light visions, and sensitivity to light, sound and movement. Migraine attacks may last from hours to days. The cause of migraine is unknown, but it is associated with some underlying diseases, as well as environmental and genetic factors. Migraine affects about 12% of population in the US.

Present methods for management and treatment of migraine include medical therapies (e.g. analgesics, triptans, ergotamines), surgery, and neurostimulation. As of today, there is no therapy to prevent or cure migraine, and a need for medical therapy focusing on the pathophysiology of migraine remains. CGRP (calcitonin gene-related peptide) vasodilatation has been associated with migraine and photophobia, which is a typical symptom of a migraine attach. Antibodies targeting CGRP may be used for treatment and/or management of migraine, e.g. as described in US Patents U.S. Pat. Nos. 9,115,194, and 9,102,731, and US Patent application US20120294802, the contents of which are herein incorporated by reference in their entirety.

In some embodiments, methods of the present disclosure may be used to treat subjects suffering from migraine. In some cases, methods of the present disclosure may be used to treat subjects suspected of developing migraine.

AAV particles and methods of using the AAV particles described in the present disclosure may be used to prevent, manage and/or treat migraine. As a non-limiting example, the AAV particles of the present disclosure comprise a nucleic acid sequence encoding at least one of the sequences described in Table 12 (SEQ ID NO: 8175-8716).

### Pain

Pain is a complex symptom associated with a variety of diseases and disorders and may be acute or chronic. Pain is challenging to treat, and many anti-pain medications have side effects, and/or they can be addictive. There remains a need for pain medications affecting the underlying pathophysiology of a pain. Antibodies for treatment for pain are on the market. For example, fasinumab (developed by Regeneron Pharmaceuticals Inc.), Fulranumab (developed by Johnson & Johnson) and tanezumab (developed by Pfizer) are antibodies against NGF (nerve growth factor) for treatment of pain, such as, osteoarthritis knee pain, chronic low back pain, bone cancer pain and/or pain associated with interstitial cystitis.

In some embodiments, methods of the present disclosure may be used to treat subjects suffering from pain. In some cases, methods of the present disclosure may be used to treat subjects suspected of developing pain.

AAV particles and methods of using the AAV particles described in the present disclosure may be used to prevent, manage and/or treat pain. As a non-limiting example, the AAV particles of the present disclosure comprise a nucleic acid sequence encoding at least one of the sequences described in Table 13 (SEQ ID NO: 8717-10586).

### Ocular Diseases

Eye is an organ comprising a number of components, including the cornea, aqueous humor, lens, vitreous humor, retina, the retinal pigment epithelium, and choroid. Ocular diseases are conditions affecting the different tissues of the eye. A number of diseases and disorders affect the different components of the eye, and may cause impaired vision, full or partial blindness, irritation, dryness, sensitivity, photophobia, and/or light aversion.

Complement in the eye has an important role in protecting the eye from infections and in modulation of the immune and inflammatory responses. In normal eye, the complement activity is at low level and is regulated by membrane bound and soluble intraocular complement regulatory proteins. Disturbance of the balance between complement activation and complement inhibition may lead to damage to self-tissue (see, e.gg, Jha et al., 2007, Mol Immunol.; 44(16): 3901-3908, and references therein). The complement system may be activated in three pathways. The classical pathway is activated by immune complexes or substances and involves e.g. complement components C1, C2, C3, C4, C3a, C5, C5a, C5b, C6, C7, C8, C9 and C5b-9. The alternative pathway activates complement component C3 when in interaction with e.g. zymosan, or lipopolysaccharide surfaces, additionally involving, e.g. Factor B, Factor Ba, Factor Bb, Factor D, and Factor P. The third activation pathway is the lectin pathway, and is related to interaction of certain serum lectins, e.g. mannose binding lectin (MBL), mannose and N-acetyl glucosamine residues present in bacterial cell walls. Complement activation is associated with a number of ocular diseases, such as, but not related, age-related macular degeneration (AMD), diabetic retinopathy, choroidal neovascularization (CNV), uveitis, diabetic macular edema, pathological myopia, von Hippel-Lindau disease, histoplasmosis of the eye, Central Retinal Vein Occlusion (CRVO), corneal neovascularization, and retinal neovascularization, choroidal neovascularization, and other ocular conditions involving complement activation. Antibodies targeting the associated complement components may be used to diagnose, manage and/or treat such ocular diseases.

Age-related macular degeneration (AMD) is a major cause of irreversible loss of central vision in the elderly worldwide. AMD leads to gradually worsening vision. AMD does not result in blindness, but may affect daily life. Wet AMD is caused by abnormal blood vessels behind the retina grow under the macula and leak blood and fluid that damage the macula. Wet AMD may be treated with laser coagulation and medication to reverse or stop the growth of blood vessels. Dry AMD is caused by break down of the light sensitive cells in the macula. As of today, there is no treatment for dry AMD.

There remains a need for prevention, management and treatment therapies for wet and dry AMD. AMD is associated with complement components, as described above. In addition, AMD is associated with proteins such as, but not limited to, VEGF (Vascular endothelial growth factor), EPO (Erythropoietin), EPOR (EPO receptor), Interleukins IL-10, IL-17A, 11-10, TNFa (tumor necrosis factor alpha), or FGFR2 (Fibroblast Growth Factor Receptor). Antibodies targeting the AMD associated complement and growth proteins may be used to treat AMD. For example, bevacizumab and ranibizumab (developed by Genentech Inc.) are antibodies targeting VEGF-A to slow down growth of new blood vessels.

Corneal diseases affect the cornea and the conjunctiva. Cornea and conjunctiva form the outer surface of the eye, which is exposed to external environment, and are susceptible to infection agents, trauma, and/or exposure to chemicals, toxins, allergens etc. Cornea is also affected by autoimmune conditions, nutritional deficiencies and cancer. Corneal diseases may cause e.g. loss of vision, blurred vision, tearing, light sensitivity and pain. Diseases affecting cornea include, but are not limited to, keratitis, corneal dystrophy, corneal degeneration, Fuchs' dystrophy, cancer of cornea, and keratoconjuctivitis. Though surgical and medical treatment therapies for corneal diseases exist, in some cases, the diseases still remain severe and may cause blindness. There remains a need to efficient therapies for prevention, management and treatment of corneal diseases. Complement components of the cornea and the conjunctiva present in a normal eye include, but are not limited to, C1, C2, C3, C4, C5, C6, C7, Factor P (properdin) and factor B. Complement may have a role in corneal diseases, and antibodies targeting complement components of the eye may be used for prevention, treatment and/or management of corneal diseases.

Uveitis is an inflammation of the uvea, comprising the iris, choroids, and ciliary body. Early symptoms include eye redness, pain, irritation and blurred vision. Uveitis may lead to transient or permanent loss of vision. Uveitis may be associated with other diseases and conditions, such as infections, systemic diseases, non-infectious and autoimmune diseases. Complement components associated with an autoimmune form of uveitis include C3b and C4b. Uveitis may be managed or treated with vitrectomy, immunosuppressive drugs, corticosteroids or cytotoxic medication. However, despite the existing therapies, autoimmune uveitis is a serious condition and may lead to full or partial blindness. There remains a need for therapies for prevention, management, and treatment of uveitis targeting pathophysiology of the disease.

Retinopathy is a disease resulting from neovascularization (excessive growth of blood vessels) in the light-sensitive tissue of the eye, retina. Retinopathy may result in impaired vision or partial or full blindness. Retinopathy may be caused by systemic diseases, e.g. diabetes, or hypertension, trauma, excessive sun light exposure or ionizing radiation. Retinopathy is often treated with laser therapy. Medical treatments, such as antibodies, to control the growth of blood vessels, are also applied. However, despite the existing treatment methods, retinopathy is still a severe condition and may lead to blindness. Diabetic retinopathy is one of the leading causes of vision loss in middle-aged individuals. There remains a need for new therapies for prevention, management and/or treatment of retinopathy. For example, antibodies targeting blood vessel growth (e.g. vascular endothelial growth factor (VEGF), complement components (e.g. C3, C4, C1q, C9, C4b), and cluster of differentiation proteins (e.g. CD55, CD59) may be used for prevention, management and/or treatment of different retinopathies.

Photophobia is a condition referring to abnormal sensitivity or aversion to light. Photophobia is related to a number of ocular and nervous system diseases and disorders. Photophobia may be caused by damage to cornea or retina, albinism, overstimulation of the photoreceptors, excessive electric pulses to the central nervous system, or optic nerve. Photophobia may be associated with migraine, nervous system disorders (e.g. autism, dyslexia, encephalitis), infections (e.g. rabies, Lyme disease, mononucleosis), eye disorders (e.g. uveitis, corneal diseases, retinal diseases, scarring or trauma to cornea). As of today, there is no medical treatment for photophobia on the market. Photophobia is associated with calcitonin gene related peptide (CGRP) and CGRP receptors, and antibodies targeting CGRP may be used to prevent and/or treat photophobia, as described in US Patent application US20120294802, the contents of which are herein incorporated by their reference.

In some embodiments, methods of the present disclosure may be used to treat subjects suffering from ocular diseases. In some cases, methods of the present disclosure may be used to treat subjects suspected of developing ocular diseases.

AAV particles and methods of using the AAV particles described in the present disclosure may be used to prevent, manage and/or treat psychiatric disorder. As a non-limiting example, the AAV particles of the present disclosure comprise a nucleic acid sequence encoding at least one of the sequences described herein.

As a non-limiting example, the AAV particles of the present disclosure comprise a nucleic acid sequence encoding at least one of the sequences described in Table 14 (SEQ ID NO: 10587-10682).

### Other Therapeutic Targets

The AAV particles or pharmaceutical compositions of the present disclosure useful in preventing or treating tauopathies or tau-associated diseases may alternatively, or in combination, encode an antibody that does not bind to the tau protein (e.g., the antigen is a polypeptide other than tau). Non-limiting examples of other target antigens include any of the following, including fragments or variants thereof, α-synuclein (monomers, oligomers, aggregates, fragments), ABCA1 (ATP-binding cassette, sub-family A, member 1), ABCA4 (ATP-binding cassette, sub-family A, member 4), ABCB1 (ATP-binding cassette, sub-family B, member 1), ACE (angiotensin I converting enzyme), ACKR1 (atypical chemokine receptor 1 (Duffy blood group)), AMPA (DL-α-amino-3-hydroxy-5-methyl-4-isoxazole propionic acid), ACTH (Adrenocorticotropic Hormone), ACVR2A (Activin receptor type-2A), ACVR2B (Activin receptor type-2B), ADDL (Adducin-Like Protein 70), ADORA2A (adenosine A2a receptor), ADRA2A (adrenoceptor alpha 2A), AIFM1 (apoptosis-inducing factor), AKT1 (RAC-alpha serine/threonine-protein kinase), ALK-1 (activin receptor-like kinase 1), Alpha beta fibril, alpha subunit (basic helix-loop-helix transcription factor), AMT (Aminomethyltransferase), Amyloid β (monomers, oligomers, aggregates, fragments), amyloid or amyloid-like proteins, ANGPTL3 (Angiopoletin-Like 3), ANGTP1 (angiopoitin 1), ANGTP2 (angiopoletin 2), ANK3 (ankyrin 3), ANKG (ankyrin G), Annexin IV, phospholipid, Anx-A1 (annexin A1), APOE (apolipoprotein E), APP (amyloid beta precursor protein), ARSD (Arylsulfatase D), ATM (Ataxia Telangiectasia Mutated serine/threonine kinase), ATXN1 (ataxin 1), ATXN2 (ataxin 2), ATXN3 (ataxin 3), ATXN7 (ataxin 7), B Lymphocyte Stimulator, BDNF (brain-derived neurotrophic factor), beta A4 peptide/Alpha beta 4, beta A4 peptide, Alpha beta 5, bAlpha beta 6, Alpha beta 7, Alpha beta 8, Alpha beta 9, Beta-secretases (BACE), BRAF (B-Raf Proto-Oncogene, Serine/Threonine Kinase), Properdin (factor P), Factors Ba and Bb, C1, C1q (complement component 1, subcomponent q), C2, C3, C4, C3a, C3b, C5, C5a, C5b, C6, C7, CB, C9 and C5b-9 (complement components), CAIX (Carbonic anhydrase IX), CA 125 (cancer antigen 125), CACNA1A (calcium channel voltage-dependent P/Q type alpha 1A subunit), cadherins, CA-IX (carbonic anhydrase 9), CALCA (calcitonin-related polypeptide alpha), CCKBR (cholecystokinin B receptor), CCL11 (eotaxin-1), CCL2 (Chemokine (C-C Motif) Ligand 2), CD11 (integrin alpha component), CD147 (basigin), CD154 (CD40L), CD19 (Cluster of Differentiation 19), CD2 (cluster of differentiation 2), CD20 (B-lymphocyte antigen), CD200 (cluster of differentiation 200), CD22 (cluster of differentiation 22), CD221 (insulin-like growth factor 1 (IGF-1) receptor), CD248 (Endosialin), CD26 (Dipeptidyl peptidase-4), CD27 (antigen precursor), CD274 (cluster of differentiation 274), CD28 (Cluster of Differentiation 28), CD29 (Integrin, Beta 1), CD3 (cluster of differentiation 3), CD30 (cluster of differentiation 30), CD31 (cluster of differentiation 31), CD33 (cluster of differentiation 33), CD37 (Leukocyte antigen), CD38 (cyclic ADP ribose hydrolase), CD3E (T-Cell Surface Antigen T3/Leu-4 Epsilon Chain), CD4 (T-Cell Surface Antigen T4/Leu. 3), CD40 (CD40 Molecule, TNF Receptor Superfamily Member 5), CD41 (Integrin, Alpha 2b (Platelet Glycoprotein IIb Of IIb/IIIa Complex, Antigen CD41)), CD44 (cluster of differentiation 44), CD51 (integrin alpha 1), CD52 (Human Epididymis-Specific Protein 5), CD55 (Decay Accelerating Factor For Complement (Cromer Blood Group)), CD58 (lymphocyte function-associated antigen 3), CD59 (MAC-inhibitory protein), CD6 (cluster of differentiation 6), CD70 (cluster of differentiation 70, ligand for CD27), CD74 (HLA class II histocompatibility antigen gamma chain), CD79B (immunoglobulin-associated beta), CEA (Carcinoembryonic antigen), CFHR1 (Complement Factor H-Related 1), CGRP (Calcitonin gene-related peptide), CHMP2B (charged multivesicular body protein 2B), CHRNA4 (cholinergic receptor nicotinic alpha 4 (neuronal)), CHRNB2 (cholinergic receptor nicotinic beta 2 (neuronal)), CISD2 (CDGSH iron sulfur domain 2), CLEC16A (C-type lectin domain family 16 member A), CLRN1 (clarin 1), CNR1 (cannabinoid receptor 1), CNTNAP2 (contactin associated protein-like 2), COMT (catechol-O-methyltransferase), CRB1 (crumbs family member 1, photoreceptor morphogenesis associated), CRX (cone-rod homeobox), CRY (crystallin), CSF1R (Colony Stimulating Factor 1 Receptor), CSF2 (Colony Stimulating Factor 2 (Granulocyte-Macrophage)), CSF2RA (Colony Stimulating Factor 2 Receptor, Alpha, Low-Affinity), CTGF (Connective Tissue Growth Factor), CTLA4 (Cytotoxic T-Lymphocyte-Associated Protein 4), CXC (chemokine receptor type 4), CXCL10 (Chemokine (C-X-C Motif) Ligand 10), DDC (dopa decarboxylase (aromatic L-amino acid decarboxylase)), DIABLO (IAP-Binding Mitochondrial Protein), differentiation factor 8 (GDF8), DISC1 (disrupted in schizophrenia 1), DLL3 (Delta-Like 3 (Drosophila)), DLL4 (Delta-Like 4 (Drosophila)), DPP4 (dipeptyl-peptidase 4), DPP6 (dipeptidyl-peptidase 6), DR6 (Death receptor 6), DRD1 (dopamine receptor D1), DRD2 (dopamine receptor D2), DRD4 (dopamine receptor D4), DRD5 (dopamine receptor 5), DRD5 (dopamine receptor D5), DTNBP1 (dystrobrevin binding protein 1), EAG1 (Ether-A-Go-Go Potassium Channel 1), EDB (fibronectin extra domain-B), EDNRA (endothelin receptor type A), EFNA1 (Ephrin-A1), EGFL7 (EGF-Like-Domain, Multiple 7), EGFR/ERBB1/HER1 (epidermal growth factor receptor 1), EN2 (Engrailed Homeobox 2), EPCAM (Epithelial cell adhesion molecule), EPHA3 (EPH Receptor A3), episialin (a carcinoma-associated mucin, MUC-1), ERBB2 (epidermal growth factor receptor 2), ERBB3 (epidermal growth factor receptor 3), ESR1 (estrogen receptor 1), F3 (coagulation factor Ill), F9 (human factor 9), F10 (human factor 10), FAAH (fatty acid amide hydrolase), Factor D C3 proactivator convertase), humanized IgG1, humanized IgG2, FAP (Fibroblast Activation Protein, Alpha), FBN2 (fibrillin 2), FBP (Folate-binding protein), FcγRIIB (Fc receptor gamma B), FcγRIIIA (Fc receptor gamma A), FLT1 (Fms-Related Tyrosine Kinase 1), FOLR1 (folate receptor alpha), Frizzled receptor, FXN (frataxin), FUS/TLS (RNA binding protein), G protein-coupled, GAA (glucosidase alpha acid), Gc-globulin (Vitamin D binding protein), Gangliosides, GD2 (ganglioside G2), GD3 (ganglioside g3), GM2 (monosialotetrahexosylganglioside 2) (GDF-8 (myostatin), GDNF (glial cell derived neurotrophic factor), GDNF (glial cell derived neurotrophic factor), GFAP (glial fibrillary acidic protein), GFRα3 (GDNF family receptor alpha-3), ghrelin, GIT1 (G protein-coupled receptor kinase interacting ArfGAP 1), GJA (Gap junction protein), GLDC Glycine Dehydrogenase (Decarboxylating), glycoprotein NMB (GPNMB), gpA33 (Glycoprotein A33 (Transmembrane)), GPC3 (glypican 3), GRIN2B (glutamate receptor ionotropic N-methyl D-aspartate 2B), GRN (granulin), GDF8 (growth differentiation factor 8), GTPases (guanosine triphosphate), GSTP1 (glutathione S-transferase pi 1), GUCA1A (guanylate cyclase activator 1A (retina), GUCY2C (anti-GCC), HMCN1 (hemicentin 1), HGF (Hepatocyte Growth Factor), HIF1A (hypoxia inducible factor 1, HINT1 (histidine triad nucleotide binding protein 1), HIST3H3 (Histone H3), histone, HLA-DQB1 (major histocompatibility complex class II DQ beta 1), HLA-DR (MHC class II cell surface receptor), HLA-DRB1 (major histocompatibility complex class II DR beta 1), hNav1.7 (sodium ion channel), HTR1A (5-hydroxytryptamine (serotonin) receptor 1A G protein-coupled), HTR2A (5-hydroxytryptamine (serotonin) receptor 2A, HTR2A (5-hydroxytryptamine (serotonin) receptor 2A G protein-coupled), HTT (huntingtin), IAP-binding mitochondrial protein, IFNAR1 (Interferon (Alpha, Beta And Omega) Receptor 1), IFNB1 (interferon beta 1 fibroblast), IFN-γ (Interferon gamma), IGF-1 receptor, IGF1R (insulin-like growth factor 1 receptor), IGF-1 (insulin-like growth factor 1), IGG1 (immunoglobulin subclass 1), IgG2 (immunoglobulin subclass 2), IgG4 (immunoglobulin subclass 4), IGHE (Immunoglobulin Heavy Constant Epsilon), IL 1B (interleukin 1 beta), IL12 (interleukin 12), IL12B (interleukin 12B), IL13 (interleukin 13), IL17A (interleukin 17A), IL17F (interleukin 17F), ILIA (interleukin 1A), IL1B (interleukin 1 beta), IL1-Ri (Interleukin 1 receptor, type 1), IL20 (Interleukin 20), IL23A (interleukin 23A), IL-23p19 subunit (interleukin 23 subunit p19), IL2RA (interleukin 2 receptor alpha), IL4R (interleukin 4 receptor alpha, IL6 (interleukin 6), IL6R (interleukin 6 receptor), IL7R (interleukin 7 receptor), ILGF2 (insulin like growth factor 2), INS (insulin), Integrin α5β1, Integrin αVβ3, integrin αIIbβ3/GPIIb/IIIa, IP6K2 (inositol hexakisphosphate kinase 2), ITGA4 (Integrin, Alpha 4 (Antigen CD49D, Alpha 4 Subunit Of VLA-4 Receptor)), ITGB7 (integrin, Alpha 7 (Antigen CD49D, Alpha 4 Subunit Of VLA-7 Receptor)), ITGAL (integrin alpha L chain), ITGAV ((Vitronectin Receptor, Alpha Polypeptide, Antigen CD51), ITGB3 (Integrin alpha-V/beta-3), KCNQ2 (potassium channel voltage gated KQT-like subfamily Q member 2), KDR (Kinase Insert Domain Receptor), KIR2D (killer immunoglobulin-like receptor (KIR) 2D subtype), KLRC1 (Killer Cell Lectin-Like Receptor Subfamily C, Member 1), LAG-3 (Lymphocyte-activation gene 3), Le (y) (Lewis y) antigen, LINGO (Leucine rich repeat and immunoglobin-like domain-containing protein 1), LOXL2 (Lysyl oxidase homolog 2), LPG (lysophosphatidylglucoside), LPS (Lipopolysaccharides), LRP1 (low density lipoprotein receptor-related protein 1), LRRC6 (Leucine Rich Repeat Containing 6), LRRK2 (leucine-rich repeat kinase 2), LTA (Lymphotoxin Alpha), MAF (maf avian musculoaponeurotic fibrosarcoma oncogene homolog), MAG (Myelin Associated Glycoprotein), MAI (myelin associated inhibitor), MAO8 (monoamine oxidase 8), MAPT (microtubule-associated protein tau), MBP (myelin basic protein), MCAF (rmonocyte chemotactic and activating factor), MCP-1 (Monocyte chemoattractant protein-1), MBL (mannose binding lectin), mannose, MET (Tyrosine-Protein Kinase Met), MIF (Macrophage Migration Inhibitory Factor (Glycosylation-inhibiting Factor), MS4A1 (Membrane. Spanning 4-Domains, Subfamily A, Member 1), MSLN (Mesothelin), MST1R (Macrophage Stimulating 1 Receptor), MSTN (myostatin), MUC1/Episialin, MUC5AC (Mucin 5AC, Oligomeric Mucus/Gel-Forming), mucin CanAg (glycoform MUC-1), Mucins, myostatin, myostatin antagonists, N-acetyl glucosamine, NCAM1 (Neural Cell Adhesion Molecule 1), Neu5Gc/NGNA (Neurogenin A), neuregulin (NRG), neurokinin B, NGF (Nerve growth factor), NMDA (N-methyl-D-aspartate), NOGO (Neurite outgrowth inhibitor), NOGO receptor-1, Nogo-66, NOGOA/NiG (Neurite Outgrowth Inhibitory Fragments of NOGOA), Notch receptor, NOTCH-1 (Notch homolog 1, translocation-associated (Drosophila)), NRG1 (neuregulin 1), NRP1 (Neuropilin 1), NT-3 trkC ligand, N-terminal region of A08-x peptide, OGG1 (8-oxoguanine DNA glycosylase), oligomers of N-terminal truncated Aβ, OPA2 (Optic Atrophy 2), OPA3 (Optic Atrophy 3), oxLDL (Oxidized low-density lipoprotein), P75 (Low-affinity Nerve Growth Factor Receptor), PAND1 9Panic disorder 1), PAND2 (Panic disorder 2), PAND39Panic disorder 3), PARK2 (parkin RBR E3 ubiquitin protein ligase), PCSK9 (proprotein convertase subtilisin/kexin type 9), PD-1 (Programmed cell death protein 1), PD-2 (Programmed cell death protein 2), PD-3 (Programmed cell death protein 3), PD-4 (Programmed cell death protein 4), PD-5 (Programmed cell death protein 5), PD-6 (Programmed cell death protein 6), PD-7 (Programmed cell death protein 7), PD-8 (Programmed cell death protein 8), PDGFRA (Platelet-derived growth factor receptor alpha), PDGFRB (Platelet-derived growth factor receptor beta), PD-L1 (Programmed cell death protein 1 ligand), PEX7 ((Peroxisomal Biogenesis Factor 7), PHOBS (phobia specific), PhosphatidyL-serine, chimeric IgG1, Phosphatide L-serine, Chimeric IgG2, PINK1 (PTEN induced putative kinase 1), platelet-derived growth factor receptor beta PDGFRB, PLAU (plasminogen activator urokinase), PLP (protelopid protein), PMP22 (peripheral myelin protein 22), POLG (polymerase (DNA directed) gamma), PRDM16 (PR domain containing 16), Prion proteins, PrP, PrPC, PrPSc, PRKCG (protein kinase C gamma), PSEN1 (presenilin 1), PSEN2 (presenilin 2), PSMA (Prostate-specific membrane antigen), PTGS2 (prostaglandin-endoperoxide synthase 2 (prostaglandin G/H synthase and cyclooxygenase)), PTPN11 (Tyrosine-protein phosphatase non-receptor type 11), PVRL4 (Poliovirus Receptor-Related 4), PVRL5 (Poliovirus Receptor-Related 5), pyroglutamated A β, RAf1 (proto-oncogene serine/threonine-protein kinase), RAGE protein, RANKL (Receptor activator of nuclear factor kappa-B ligand), RCAN1 (regulator of calcineurin 1), RDh12 (retinol dehydrogenase 12 (all-trans/9-cis/11-cis), RGM A (Repulsive guidance molecule A), RHD (Rh blood group, D antigen), RHO (rhodopsin), RPE65 (retinal pigment epithelium-specific protein 65 kDa), RTN4 (Reticulon-4, NOGO), S100B (calcium-binding protein B), S1P4 (Type 4 sphingosine I-phosphate G protein-coupled receptor), SCN1A (Sodium Channel, Voltage Gated, Type I Alpha Subunit), SDC1 (Syndecan 1), selectin P, SHANK3 (SH3 And Multiple Ankyrin Repeat Domains 3), SLAMF7 (SLAM Family Member 7), SLC18A2 (solute carrier family 18 (vesicular monoamine transporter, member 2), SLC1A2 (solute carrier family 1 (glial high affinity glutamate transporter, member 2), SLC34A2 (Solute Carrier Family 34 (Type II Sodium/Phosphate Cotransporter), SLC6A3 (solute carrier family 6 (neurotransmitter transporter) member 3), SLC6A4 (Solute Carrier Family 6 (Neurotransmitter Transporter), SMN1 (survival of motor neuron 1 telomeric), SMN2 (survival of motor neuron 2 centromeric), SNCA (synuclein alpha (non A4 component of amyloid precursor)), SNCA (synuclein alpha (non A4 component of amyloid precursor), SNCB (synuclein beta), SOD1 (superoxide dismutase 1 soluble), SOST (Sclerostin), sphingosine-1-phosphate, SQSTM1 (sequestosome 1), STEAP1 (Six Transmembrane Epithelial Antigen Of The Prostate 1), SULF2 (Sulfatase 2), TACR1 (tachykinin receptor 1), TAG-72 (Tumor-associated glycoprotein 72), TARDBP (TAR DNA binding protein), tau antigen, tau protein, tau pS422, TDP-43, tenascin, tenascin C, TFPI (Tissue Factor Pathway Inhibitor (Lipoprotein-Associated Coagulation Inhibitor)), TGF beta (Transforming growth factor beta), TH (Tyrosine hydroxylase), TkrC (Tropomyosin receptor kinase C), TMEFF2 (Transmembrane Protein With EGF-Like And Two Follistatin-Like Domains 2), TMEFF3 (Transmembrane Protein With EGF-Like And Two Follistatin-Like Domains 3), TNF (tumor necrosis factor), TNFa (tumor necrosis factor alpha), TNFRSF10B (Tumor Necrosis Factor Receptor Superfamily, Member 10b), TNFRSF12A (Tumor Necrosis Factor Receptor Superfamily, Member 12A), TNFRSF8 (Tumor Necrosis Factor Receptor Superfamily, Member 8), TNFRSF9 (Tumor Necrosis Factor Receptor Superfamily, Member 9), TNFSF11 (Tumor Necrosis Factor Receptor Superfamily, Member 11), TNFSF13B (Tumor Necrosis Factor Receptor Superfamily, Member 13b), TNF-α (Tumor Necrosis Factor alpha), TNNT2 (troponin T type 2), TOR1A (torsin family 1 member A (torsin A)), TPBG (Trophoblast Glycoprotein), TPH2 (tryptophan hydroxylase 2), TRAILR1 (Death receptor 4), TRAILR2 (Death receptor 5), TrkA (Tropomyosin receptor kinase A), TRPV4 (Transient Receptor Potential Cation Channel, Subfamily V, Member 4), TSC2 (tuberous sclerosis 2), TULP1 (tubby like protein 1), tumor necrosis factor related protein 5, tumor specific glycosylation of MUC1, tumor-associated calcium signal transducer 2, tumor protein p53, TYRP1 (glycoprotein 75), UCHI1 (ubiquitin carboxyl-terminal esterase L1 (ubiquitin thiolesterase), UNC-13A (unc-13 homolog A), USH1C (Usher Syndrome 1C), USH2A (Usher Syndrome 2A (Autosomal Recessive, Mild), VEGF (Vascular endothelial growth factor), VEGF A (Vascular endothelial growth factor A), C5, Factor P, Factor D, EPO (Erythropoletin), EPOR (EPO receptor), Interleukins, IL-1β, IL-17A, 11-10, TNFα, FGFR2 (Fibroblast Growth Factor Receptor 2), VEGFR (vascular endothelial growth factor receptor), VEGFR2 (vascular endothelial growth factor receptor 2), vimentin, voltage gated ion channels, VWF (Von Willebrand Factor), WFS1 (Wolfram syndrome 1 (wolframin)), YES1 (Yamaguchi Sarcoma Viral Oncogene Homolog 1).

In some embodiments, the AAV particle of the present disclosure, useful in treating a non-infectious disease, targets an antigen considered to be useful in the treatment of a different disease. As a non-limiting example, an AAV particle or pharmaceutical composition thereof used for the treatment of cancer, immune system dysfunctions or inflammatory disease may likewise be used for the treatment of a neurodegenerative disorder such as, but not limited to, AD, PD, HD, ALS, SMA, or DLB.

Multiple Specific Diseases and/or Targets

In some embodiments, methods of the present disclosure may be used to treat subjects suffering from a disease, disorder, and/or condition that may be associated with one or multiple disease-related epitopes and/or targets. In certain embodiments, the AAV particle of the present disclosure targets one or more antigens considered to be useful in the treatment of such a disease. As a non-limiting example, an AAV particle or pharmaceutical composition thereof used for the treatment of one or multiple diseases may be associated with one or multiple disease-related epitopes and/or targets that may comprise antibodies that are multispecific. In some cases, methods of the present disclosure may be used to treat subjects suspected of developing a disease associated with multiple disease-related epitopes and/or targets.

AAV particles and methods of using the AAV particles described in the present disclosure may be used to prevent, manage, and/or treat disease associated with multiple disease-related epitopes and/or targets. As a non-limiting example, the AAV particles of the present disclosure comprise a nucleic acid sequence encoding at least one of the sequences described in Table 16 (SEQ ID NO: 10764-10916).

As a non-limiting example, the AAV particles of the present disclosure comprise a nucleic acid sequence encoding at least one of the sequences described in Table 15 (SEQ ID NO: 10683-10763).

### Diagnostic Applications

The AAV particles of the present disclosure may be used for diagnostic purposes or as diagnostic tools for any of the aforementioned diseases or disorders. As a non-limiting example, the AAV particles of the present disclosure or the antibodies encoded within the viral genome therein may be used as a biomarker for disease diagnosis. As a second non-limiting example, the AAV particles of the present disclosure or the antibodies encoded within the viral genome therein may be used for diagnostic imaging purposes, e.g., MRI, PET, CT or ultrasound.

### Preventative Applications

The AAV particles of the present disclosure or the antibodies encoded by the viral genome therein may be used to prevent disease or stabilize the progression of disease. In some embodiments, the AAV particles of the present disclosure are used as a prophylactic to prevent a disease or disorder in the future. In some embodiments, the AAV particles of the present disclosure are used to halt further progression of a disease or disorder. As a non-limiting example, the AAV particles of the disclosure may be used as, and/or in a manner similar to that of a vaccine. As a non-limiting example of the payload of the present disclosure may encode Influenza associated antibodies such as, but not limited to SD36, SD38, SD83, and SD84 and/or sdAbs SD38.SD36, MD2407 and MD3606 as described by Laursen et al. Science 2018:Vol. 362, Issue 6414, pp. 598.602 for preventing and/or stabilizing the progression of Influenza.

The AAV particles of the present disclosure and/or the antibodies encoded by the viral genome therein may be used as a contraceptive. As used herein, the term, “contraceptive” may be defined as any agent or method that may be used to prevent pregnancy. In some embodiments, the contraceptive may be used short-term or long-term. The contraceptive may be reversible or permanent. In one embodiments, the antibodies of the present disclosure may bind to human CD52. In some embodiments, the antibody may bind to a carbohydrate epitope expressed specifically on CD52 in the human male reproductive system. In some embodiments, the antibodies of the present disclosure may bind to epitopes in targets such as but not limited to gonadotropin-releasing hormone (GnRH), luteinizing hormone (LH), Zona pellucida (ZP), follicle-stimulating hormone (FSH), chorionic gonadotropin, Anti-Mullerian Hormone/Mullerian Inhibiting Substance (AMH/MIS) and/or testosterone.

### Research Applications

The AAV particles of the present disclosure or the antibodies encoded by the viral genome therein may also be used as research tools. The AAV particles of the disclosure may be used as in any research experiment, e.g., in vivo or in vitro experiments. In a non-limiting example, the AAV particles of the disclosure may be used in cultured cells. The cultured cells may be derived from any origin known to one with skill in the art, and may be as non-limiting examples, derived from a stable cell line, an animal model or a human patient or control subject. In a non-limiting example, the AAV particles of the disclosure may be used in in vivo experiments in animal models (i.e., mouse, rat, rabbit, dog, cat, non-human primate, guinea pig, ferret, c-elegans, drosophila, zebrafish, or any other animal used for research purposes, known in the art). In another non-limiting example, the AAV particles of the disclosure may be used in human research experiments or human clinical trials.

### Combination Applications

The AAV particles of the disclosure may be used as a combination therapy with any other therapeutic molecule known in the art. The therapeutic molecule may be approved by the US Food and Drug Administration or may be in clinical trial or at the preclinical research stage. The therapeutic molecule may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

As a non-limiting example, AAV particles encoding antibody BAN2401 or 158, or fragments thereof may be used in combination therapy with therapeutic molecules such as but not limited to beta amyloid cleaving enzyme (BACE) inhibitor e.g. Elenbecestat.

### V. Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles.

In some embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677 and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In some embodiments, the AAV particles may be delivered using a cannula which is MRI-compatible. Non-limiting examples of MRI-compatible cannulas include those taught in International Patent Publication No. WO2011130107, the contents of which are herein incorporated by reference in its entirety.

In some embodiments, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### VI. Definitions

At various places in the present specification, substituents of compounds of the present disclosure are disclosed in groups or in ranges. It is specifically intended that the present disclosure include each and every individual subcombination of the members of such groups and ranges.

About: As used herein, the term “about” means+/−10% of the recited value.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which comprises a viral genome with at least one payload region and at least one ITR region. V vectors of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the disclosure may have activity and this activity may involve one or more biological events.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include V_{H} and V_{L} domains, scFvs, Fab, Fab′, F(ab′), Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes or antibodies to innate defense molecules.

Antibody-based composition: As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%,6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may effect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle.

Chimeric antigen receptor (CAR): As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. As a non-limiting example, the ASTR of a CAR may be any of the antibodies listed in Tables 3-16, antibody-based compositions or fragments thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Comprehensive Positional Evolution (CPE™): As used herein, the term “comprehensive positional evolution” refers to an antibody evolution technology that allows for mapping of the effects of amino acid changes at every position along an antibody variable domain's sequence. This comprehensive mutagenesis technology can be used to enhance one or more antibody properties or characteristics.

Comprehensive Protein Synthesis (CPS™): As used herein, the term “comprehensive protein synthesis” refers to a combinatorial protein synthesis technology that can be used to optimize antibody properties or characteristics by combining the best properties into a new, high-performance antibody.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Epitope: As used herein, an “epitope” refers to a surface or region on a molecule that is capable of interacting with a biomolecule. For example, a protein may contain one or more amino acids, e.g., an epitope, which interacts with an antibody, e.g., a biomolecule. In some embodiments, when referring to a protein or protein module, an epitope may comprise a linear stretch of amino acids or a three-dimensional structure formed by folded amino acid chains.

EvoMap™: As used herein, an EvoMap™ refers to a map of a polypeptide, wherein detailed informatics are presented about the effects of single amino acid mutations within the length of the polypeptide and their influence on the properties and characteristics of that polypeptide.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%,75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4 The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)

Infectious disease: As used herein, the term “infectious disease” refers to any disorder and/or condition caused by invasion into the body of an exogenous organism or infection agent that is not typically present such as, but not limited to, viruses, bacteria, prions, nematodes, fungus, parasites or arthropods.

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules, such as a W chain and V_{L} chain or an antibody. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (mRNA) binding site represents a nucleotide location or region of a nucleic acid transcript to which at least the “seed” region of a miRNA binds.

Modified: As used herein “modified” refers to a changed state or structure of a molecule of the disclosure. Molecules may be modified in many ways including chemically, structurally, and functionally.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5 terminus, but does not include the 3′ terminus. In some embodiments, 3′ regions may comprise any length of nucleic acids, which include the 3′ terminus, but does not comprise the 5 terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference V sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” is a polynucleotide comprising at least one inverted terminal repeat (ITR) and at least one encoded payload. A viral genome encodes at least one copy of the payload.

Described herein are compositions, methods, processes, kits and devices for the design, preparation, manufacture and/or formulation of AAV particles. In some embodiments, payloads, such as but not limited to AAV polynucleotides, may be encoded by payload constructs or contained within plasmids or vectors or recombinant adeno-associated viruses (AAVs).

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### VII. Examples

### Example 1. Production and Purification of AAV Particles

AAV particles described herein may be produced using methods known in the art, such as, for example, triple transfection or baculovirus mediated virus production. Any suitable permissive or packaging cell known in the art may be employed to produce the vectors. Mammalian cells are often preferred. Also preferred are trans-complementing packaging cell lines that provide functions deleted from a replication-defective helper virus, e.g., 293 cells or other Ela trans-complementing cells.

The gene cassette may contain some or all of the parvovirus (e.g., AAV) cap and rep genes. Preferably, however, some or all of the cap and rep functions are provided in trans by introducing a packaging vector(s) encoding the capsid and/or Rep proteins into the cell. Most preferably, the gene cassette does not encode the capsid or Rep proteins. Alternatively, a packaging cell line is used that is stably transformed to express the cap and/or rep genes

Recombinant AAV virus particles are, in some cases, produced and purified from culture supernatants according to the procedure as described in US20160032254, the contents of which are incorporated by reference. Production may also involve methods known in the art including those using 293T cell, sf9 insect cells, triple transfection or any suitable production method.

In some cases, 293 cells are transfected with CaPO4 with plasmids required for production of AAV, i.e., AAV2 rep, an adenoviral helper construct and a ITR flanked transgene cassette. The AAV2 rep plasmid also contains the cap sequence of the particular virus being studied. Twenty-four hours after transfection, which occurs in serum containing DMEM, the medium is replaced with fresh medium with or without serum. Three (3) days after transfection, a sample is taken from the culture medium of the 293 adherent cells. Subsequently cells are scraped and transferred into a receptacle. After centrifugation to remove cellular pellet, a second sample is taken from the supernatant after scraping. Next cell lysis is achieved by three consecutive freeze-thaw cycles (−80 C. to 37 C.). Cellular debris is removed and sample 3 is taken from the medium. The samples are quantified for AAV particles by DNase resistant genome titration by Taqman™ PCR. The total production yield from such a transfection is equal to the particle concentration from sample 3.

AAV vector titers are measured according to genome copy number (genome particles per milliliter). Genome particle concentrations are based on Taqman® PCR of the vector DNA as previously reported (Clark et al, (1999) Hum. Gene Ther., 10:1031-1039; Veldwijk et al. (2002) Mol. Ther., 6:272-278).

### Example 2. Tissue Specific Expression

To evaluate the expression of various encoded antibody payloads in tissues, a series of AAV particles carrying the encoded antibody sequences driven by a panel of ubiquitous and tissue-specific promoters are made. These particles are administered to the specific tissue, e.g., intramuscularly, via an appropriate route, e.g., a single injection in the gastrocnemius muscle and expression is monitored to determine the relative expression potential of the payload as well as of each promoter in this target tissue. Measurement of antibody production is performed using standard techniques, for example by ELISA.

In some cases, the cytomegalovirus immediate early promoter (CMV), chimeric chicken-beta-actin (CAG), and ubiquitin C (UBC), CBA, H1 promoters provide robust expression.

### Example 3. Generation of Antibodies

### Antibody Production by Hybridoma Technology

Host animals (e.g. mice, rabbits, goats, and llamas) are immunized by an injection with an antigenic protein to elicit lymphocytes that specifically bind to the antigen. Lymphocytes are collected and fused with immortalized cell lines to generate hybridomas. Hybridomas are cultured in a suitable culture medium that is enriched with appropriate selection agents to promote growth.

Antibodies produced by the cultured hybridomas are subjected to analysis to determine binding specificity of the antibodies for the target antigen. Once antibodies with desirable characteristics are identified, corresponding hybridomas are subcloned through limiting dilution procedures and grown by standard methods. Antibodies produced by these cells are isolated and purified using standard immunoglobulin purification procedures.

### Recombinant Antibody Production

Recombinant antibodies are produced using heavy and light chain variable region cDNA sequences selected from hybridomas or from other sources. Sequences encoding antibody variable domains expressed by hybridomas are determined by extracting RNA molecules from antibody-producing hybridoma cells and producing cDNA by reverse transcriptase polymerase chain reaction (PCR). PCR is used to amplify cDNA using primers specific for heavy and light chain sequences. PCR products are then subcloned into plasmids for sequence analysis. Antibodies are produced by insertion of resulting variable domain sequences into expression vectors.

Recombinant antibodies are also produced using phage display technology. Target antigens are screened, in vitro, using phage display libraries having millions to billions of phage particles expressing unique single chain variable fragments (scFvs) on their viral coat. Precipitated phage particles are analyzed and sequences encoding expressed scFvs are determined. Sequences encoding antibody variable domains and/or CDRs are inserted into expression vectors for antibody production.

Recombinant antibodies are further produced using yeast surface display technology, wherein antibody variable domain sequences are expressed on the cell surface of Saccharomyces cerevisiae. Recombinant antibodies are developed by displaying the antibody fragment of interest as a fusion to e.g. Aga2p protein on the surface of the yeast, where the protein interacts with proteins and small molecules in a solution. scFvs with affinity towards desired receptors are isolated from the yeast surface using magnetic separation and flow cytometry. Several cycles of yeast surface display and isolation will be done to attain scFvs with desired properties through directed evolution.

### Example 4. Optimization of the Encoded Antibody

To design an optimal framework for the expression of an antibody, the heavy and light chains of several antibodies separated by an F2A self-processing peptide sequence are cloned into a mammalian expression vector under the control of the CMV promoter. 293T cells or any suitable cell line transfected with these vectors exhibit secretion of human IgG into the culture supernatant that is then detected by ELISA.

To increase expression, the antibody chains and/or the processing peptide are codon optimized for mammalian expression. In some instances, a furin cleavage site at the N-terminus is inserted for better processing.

To improve secretion of the antibody, the endogenous signal sequences are replaced with a sequence which may or may not be codon optimized, derived from any gene. In some cases, the human growth hormone signal sequence is used. Any of the heavy, light or both chains may be driven by any signal sequence, whether the same or different. Antibody expression is confirmed using standard immunohistochemical techniques, including ELISA.

### Example 5. Vectored Antibodies

Viral genomes are designed for AAV delivery of antibodies to cells. The viral genome comprises a payload region and at least one inverted terminal repeat (ITR) region. The payload region may optionally encode regulatory elements e.g., a promoter region, an intronic region, or a polyadenylation sequence. The payload region comprises a sequence encoding one or more polypeptides selected from the group consisting of those listed in Table 3. An exemplary payload region comprises a sequence encoding an antibody heavy chain, a region encoding an antibody light chain and a region encoding a linker connecting the heavy and light chain sequences or polypeptides before further processing. A promoter is selected to target the desired tissue or for desired regulation of expression, or both. The promoter may be selected from human EF1α, CMV, CBA, and its derivative CAG, GUSB, UBC, or any other promoter known to one with skill in the art, or combinations thereof. The 5′ and 3′ ITRs may or may not be of the same serotype as the capsid of the AAV particle.

Payload regions may optionally encode a linker between light and heavy antibody chain sequences or polypeptides. Sequence encoding linkers are derived from an internal ribosome entry site (IRES; SEQ ID NO: 1732), foot and mouth disease virus 2A (F2A; SEQ ID NO: 1727), porcine teschovirus-1 virus 2A (P2A; SEQ ID NO: 1728), a furin cleavage site (Furin; SEQ ID NO: 1724), or a 5xG4S (SEQ ID NO: 1729; “5xG4S” peptide sequence disclosed as SEQ ID NO: 13144) linker sequence. In various payload regions, the order of heavy and light chains is alternated with respect to 5′ to 3′ direction. Payloads are further designed to encode protein signal sequences (to aid in protein processing, localization, and/or secretion) as well as an untranslated poly A tail.

Each viral genome is then incorporated into an AAV cloning vector to create payload expression vectors.

The payload expression vectors are expressed in e.g. Expi 293 cells. The supernatants are collected and expressed antibodies are purified using protein AG beads. Supernatants are diluted with a loading buffer and applied to a column prepared with A/G beads. Unbound proteins are washed through with loading buffer. Elution buffer is added to the column, fractions collected, and fractions containing proteins of interest are identified with absorption spectroscopy technique, pooled together, and neutralized. Western blotting techniques are used to identify payload regions producing the antibody proteins of interest. Purified antibodies are then tested for their affinity to their specific target by e.g. ELISA essay technique and antibodies with the highest affinity are identified and selected.

Finally, the rAAVs are produced using, for example, HEK293T cells. The cells are transfected simultaneously with the viral genome of the present disclosure, a viral genome encoding helper proteins and a viral genome encoding replication and capsid proteins.

### Example 6. In Vivo Expression and Efficacy of Antibody Payloads

To determine the efficacy or comparative expression of encoded antibodies, dose-dependent expression is determined at a series of time points. Samples from mice treated with AAV particles encoding antibodies or luciferase at various levels are examined for expression using standard techniques such as nucleic acid analyses for RNA levels, protein analyses for antibody levels and compared to the expression of the luciferase control.

### Example 7. Treatment of Non-Infectious Disease

AAV particles of the current disclosure encoding an antibody are administered to a patient who has been diagnosed with a non-infectious disease, disorder or condition. The non-infectious disease, disorder or condition may be e.g. a central nervous system disease, muscular disease, neuropathy, psychiatric disorder, ocular disease, pain disorder, migraine, cancer, systemic disease, inflammation, or an immune system disease. The purpose of the treatment may be aimed to manage the disease, prevent or slow the progression of the disease, treat the symptoms associated with the disease and/or cure the disease.

The AAV particles may be administered through an intramuscular injection to the skeletal muscle. The administration may include one or more injections over a period of time. The level and distribution of AAV particles and antibody expression is monitored by standard diagnostic techniques known in the art. Such diagnostic techniques include e.g. (e.g. from blood, urine, or saliva), cerebrospinal fluid (CSF) testing, or any other testing useful for monitoring antibody levels in the body.

Additionally, the progression of the disease and the health of the patient is monitored by standard diagnostic techniques known in the art. Such techniques may include diagnostic imaging (e.g. X-ray, MRA scans, Ultrasound scans, PET scans, Nuclear scans, mammography), biopsy, laboratory tests (e.g. from blood, urine, or saliva), cerebrospinal fluid (CSF) testing, vital signs, clinical tests (cognitive, motor or reflex tests) and other relevant techniques. Treatment with the AAV particles may results in cure of the non-infectious disease, slowing down or stabilizing the progression of the disease, or have no effect on the progression of the disease. Additionally, the treatment may reduce severity of one or more symptoms associated with the disease, eliminate one or more symptoms associated with the disease or have no effect on the symptoms.

### VIII. Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with reference to the appended claims so as to provide the broadest possible interpretation of such claims in view of the prior art and, therefore, to effectively encompass the intended scope of the disclosure.

1. An AAV particle comprising a capsid and a viral genome, said viral genome comprising a 5′ inverted terminal repeat (ITR) sequence region, at least one promoter sequence region, a polyA sequence region, a 3′ITR sequence region, and at least one payload region comprising a first nucleic acid sequence encoding an antibody, an antibody fragment or an antibody variant, wherein the 5′ITR sequence region comprises SEQ ID NO: 13519 or 13520, wherein the 3′ITR sequence region comprises SEQ ID NO: 13521 or 13522, wherein the at least one promoter sequence region comprises one or more of SEQ ID NO: 13523-13534, and wherein the polyA sequence region comprises SEQ ID NO: 13576, 13577, or 13578.

2. The AAV particle of claim 1, wherein the 5′ITR sequence region comprises SEQ ID NO: 13519, the 3′ITR sequence region comprises SEQ ID NO: 13521, and the polyA sequence region comprises SEQ ID NO: 13576.

3. The AAV particle of claim 1, wherein the 5′ITR sequence region comprises SEQ ID NO: 13519, the 3′ITR sequence region comprises SEQ ID NO: 13521, and the polyA sequence region comprises SEQ ID NO: 13577.

4. The AAV particle of claim 1, wherein the 5′ITR sequence region comprises SEQ ID NO: 13520, the 3′ITR sequence region comprises SEQ ID NO: 13522, and the polyA sequence region comprises SEQ ID NO: 13576.

5. The AAV particle of claim 1, wherein the 5′ITR sequence region comprises SEQ ID NO: 13520, the 3′ITR sequence region comprises SEQ ID NO: 13522, and the polyA sequence region comprises SEQ ID NO: 13577.

6. The AAV particle of any of claims 1-5, wherein the at least one promoter sequence comprises SEQ ID NO: 13523.

7. The AAV particle of any of claims 1-5, wherein the at least one promoter sequence comprises SEQ ID NO: 13524.

8. The AAV particle of any of claims 1-5, wherein the at least one promoter sequence comprises SEQ ID NO: 13524 and 13525.

9. The AAV particle of any of claims 1-5, wherein the at least one promoter sequence comprises SEQ ID NO: 13525.

10. The AAV particle of any of claims 1-5, wherein the at least one promoter sequence comprises SEQ ID NO: 13526.

11. The AAV particle of any of claims 1-5, wherein the at least one promoter sequence comprises SEQ ID NO: 13527.

12. The AAV particle of any of claims 1-5, wherein the at least one promoter sequence comprises SEQ ID NO: 13528.

13. The AAV particle of any of claims 1-5, wherein the at least one promoter sequence comprises SEQ ID NO: 13529.

14. The AAV particle of any of claims 1-5, wherein the at least one promoter sequence comprises SEQ ID NO: 13530.

15. The AAV particle of any of claims 1-5, wherein the at least one promoter sequence comprises SEQ ID NO: 13531.

16. The AAV particle of any of claims 1-5, wherein the at least one promoter sequence comprises SEQ ID NO: 13532.

17. The AAV particle of any of claims 1-5, wherein the at least one promoter sequence comprises SEQ ID NO: 13533.

18. The AAV particle of any of claims 1-5, wherein the at least one promoter sequence comprises SEQ ID NO: 13534.

19. The AAV particle of any of claims 1-18, wherein the viral genome comprises at least one intron sequence region.

20. The AAV particle of claim 19, wherein the at least one intron sequence region comprises any of SEQ ID NO: 13540-13554.

21. The AAV particle of claim 19, wherein the viral genome comprises at least one exon sequence region.

22. The AAV particle of claim 21, wherein the at least one exon sequence region comprises any of SEQ ID NO: 13535-13539.

23. The AAV particle of claim 21, wherein the viral genome comprises two intron sequence regions and two exon sequence regions.

24. The AAV particle of claim 23, wherein at least one of the intron sequence regions comprise any of SEQ ID NO: 13540-13554 and at least one of the exon sequence regions comprise any of SEQ ID NO: 13535-13539.

25. The AAV particle of any of claims 1-24, wherein the viral genome comprises a filler sequence region.

26. The AAV particle of claim 25, wherein the filler sequence region comprises any of SEQ ID NO: 13579 or 13580.

27. The AAV particle of any of claims 1-26, wherein the viral genome comprises a tag sequence region.

28. The AAV particle of claim 27, wherein the tag sequence region comprises any of SEQ ID NO: 13571-13575.

29. The AAV particle of any of claims 1-28, wherein the viral genome comprises at least one signal sequence region.

30. The AAV particle of claim 29, wherein the at least one signal sequence region comprises any of SEQ ID NO: 13555-13570.

31. The AAV particle of any of claims 1-30, wherein the first nucleic acid sequence is selected from the group consisting of SEQ ID NO: 1740-1840, 1984-1992, 1996-2014, 2021-2041, 2048-2073, 2099-2120, 3912, 3913, 4002, 4003, 4053-4055, 4872-4874, 5684-5697, 7399, 7401, 7412-7414, 7576, 7602-7616, 7622-7632, 7697-7702, 7772-7780, 7788-7791, 7803-7804, 7813, 7820-7823, 7882-7884, 8042-8047, 8061-8067, 8375-8404, 8454-8490, 8545-4569, 8620-4645, 8717-8770, 9237-9271, 9470-9474, 9480-9529, 9585-9636, 9803-9988, and 10210-10392, said first nucleic acid segment encoding one or more polypeptides, wherein the one or more polypeptides are independently selected from the group consisting of SEQ ID NO: 1841-1983,1993-1995, 2015-2020, 2042-2047, 2074-2098, 2121-3911, 3914-4001, 4004-4052, 4056-4871, 4875-5683, 5698-7398, 7402-7411, 7415-7575, 7577-7601, 7617-7621, 7633-7696, 7703-7771, 7781-7787, 7792-7802, 7805-7812, 7814-7819, 7824-8041, 8048-8060, 8068-8174, 8221-8374, 8405-8453, 8491-8544, 8570-8619, 8646-8716, 8771-9236, 9272-9469, 9475-9479, 9530-9584, 9634-9802, 9989-10209, 10393-10916 and 13165-13518, and fragments thereof.

32. The AAV particle of any of claims 1-30, wherein the first nucleic acid sequence is selected from the group consisting of SEQ ID NO: 1740-1840, 1984-1992, 1996-2014, 2021-2041, 2048-2073, 2099.2120, 3912, 3913, 4002, 4003, 4053-4055, 4872-4874, 5684-5697, 7399, 7401, 7412-7414, 7576, 7602-7616, 7622-7632, 7697-7702, 7772-7780, 7788-7791, 7803-7804, 7813, 7820, 7823, 7882, 7884, 8042, 8047, 8061-8067, 8375-8404, 8454-8490, 8545-4569, 8620-8645, 8717-8770, 9237-9271, 9470-9474, 9480-9529, 9585-9636, 9803-9988, and 10210-10392, said first nucleic acid segment encoding a bispecific antibody derived from SEQ ID NO: 1841-1983,1993-1995, 2015-2020, 2042-2047, 2074-2098, 2121-3911, 3914-4001, 4004-4052, 4056-4871, 4875-5683, 5698-7398, 7402-7411, 7415-7575, 7577-7601, 7617-7621, 7633-7696, 7703-7771, 7781-7787, 7792-7802, 7805-7812, 7814-7819, 7824-8041, 8048-8060, 8068-8174, 8221-8374, 8405-8453, 8491-8544, 8570-8619, 8646-8716, 8771-9236, 9272-9469, 9475-9479, 9530-9584, 9634-9802, 9989-10209, 10393-10916 and 13165-13518, and fragments thereof.

33. The AAV particle of claim 31 or 32, wherein the first nucleic acid sequence is codon optimized.

34. The AAV particle of any of claims 31-33, wherein the first nucleic acid segment encodes one or more polypeptides selected from the group consisting of an antibody heavy chain, an antibody light chain, a linker, and combinations thereof.

35. The AAV particle of claim 34, wherein any of the polypeptides encoded by first nucleic acid segment of the payload region is humanized.

36. The AAV particle of claim 34, wherein the linker comprises any of SEQ ID NO: 1724-1739 and 13151-13162.

37. The AAV particle of claim 34, wherein the first nucleic acid segment encodes from 5 to 3′, an antibody heavy chain, a linker, and an antibody light chain.

38. The AAV particle of claim 34, wherein the first nucleic acid segment encodes from 5 to 3′, an antibody light chain, a linker, and an antibody heavy chain.

39. The AAV particle of claim 34, wherein the first nucleic acid segment encodes one or more antibody heavy chains.

40. The AAV particle of claim 39, wherein the first nucleic acid segment encodes one or more antibody heavy chain sequences listed in Tables 3-16, and fragments thereof.

41. The AAV particle of claim 34, wherein the first nucleic acid segment encodes one or more antibody light chains.

42. The AAV particle of claim 41, wherein the first nucleic acid segment encodes one or more antibody light chain sequences listed in Tables 3-16 and fragments thereof.

43. The AAV particle of claim 34, wherein the first nucleic acid segment encodes one or more antibody heavy chains and one or more antibody light chains and, optionally one or more linkers.

44. The AAV particle of any of claims 34-43, wherein the linker comprises any of SEQ ID NO: 1724-1739 and 13151-13162.

45. The AAV particle of claim 31, wherein the first nucleic acid segment encodes an antibody, having at least 95% identity to any of the sequences selected from the group consisting of SEQ ID NO: 1740-10916 and 13165-13518.

46. The AAV particle of claim 32, wherein the first nucleic acid segment encodes an bispecific antibody, having at least 95% identity to any of the sequences selected from the group consisting of SEQ ID NO: 1740-10916 and 13165-13518.

47. The AAV particle of any of claims 1-46, wherein the payload region of the viral genome comprises a second nucleic acid segment, said second nucleic acid segment encoding an aptamer, siRNA, saRNA, ribozyme, microRNA, mRNA or combination thereof.

48. The AAV particle of claim 47, wherein the second nucleic acid segment encodes an siRNA and said siRNA is designed to target the mRNA that encodes the target of the antibody encoded by the first nucleic acid segment.

49. The AAV particle of claim 47, wherein the second nucleic acid segment encodes a microRNA and said microRNA is selected to target the mRNA that encodes the target of the antibody encoded by the first nucleic acid segment.

50. The AAV particle of claim 47, wherein the second nucleic acid segment encodes an mRNA and said mRNA encodes one or more peptides inhibitors of the same target of the antibody encoded by the first nucleic acid segment.

51. The AAV particle of any of claims 47-50, wherein the payload region of the viral genome comprises a third nucleic acid segment.

52. The AAV particle of claim 51, wherein the third nucleic acid segment encodes a nuclear export signal.

53. The AAV particle of claim 51, wherein the third nucleic acid segment encodes a polynucleotide or polypeptide which acts as a regulator of expression of the viral genome in which it is encoded.

54. The AAV particle of claim 51, wherein the third nucleic acid segment encodes a polynucleotide or polypeptide which acts as a regulator of expression of the payload region of the viral genome in which it is encoded.

55. The AAV particle of claim 51, wherein the third nucleic acid segment encodes a polynucleotide or polypeptide which acts as a regulator of expression of the first nucleic acid segment of the payload region of the viral genome in which it is encoded.

56. The AAV particle of any of claims 1-55, wherein the viral genome is single stranded.

57. The AAV particle of any of claims 1-55, wherein the viral genome is self-complementary.

58. The AAV particle of any of claims 1-57, wherein the capsid is a serotype selected from the group of consisting of SEQ ID NO: 1-1261 and 10928-13139.

59. An AAV particle comprising a capsid and a viral genome, said viral genome comprising at least one inverted terminal repeat (ITR) region and a payload region, said payload region comprising a regulatory sequence operably linked to at least a first nucleic acid segment, wherein the first nucleic segment is selected from the group consisting of SEQ ID NO: 1740-1840, 1984-1992, 1996-2014, 2021-2041, 2048-2073, 2099-2120, 3912, 3913, 4002, 4003, 4053-4055, 4872-4874, 5684-5697, 7399, 7401, 7412-7414, 7576, 7602-7616, 7622-7632, 7697-7702, 7772-7780, 7788-7791, 7803-7804, 7813, 7820-7823, 7882-7884, 8042-8047, 8061-8067, 8375-4404, 8454-8490, 8545-4569, 8620-86458717-8770, 9237-9271, 9470-9474, 9480-9529, 9585-9636, 9803-9988, and 10210-10392, said first nucleic acid segment encoding one or more polypeptides, wherein the one or more polypeptides are independently selected from the group consisting of SEQ ID NO: 1841-1983, 1993-1995, 2015-2020, 2042-2047, 2074-2098, 2121-3911, 3914-4001, 4004-4052, 4056-4871, 4875-5683, 5698-7398, 7402-7411, 7415-7575, 7577-7601, 7617-7621, 7633-7696, 7703-7771, 7781-7787, 7792-7802, 7805-7812, 7814-7819, 7824-8041, 8048-8060, 8068-8174, 8221-8374, 8405-8453, 8491-8544, 8570-8619, 8646-8716, 8771-9236, 9272-9469, 9475-9479, 9530-9584, 9634-9802, 9989-10209, 10393-10916 and 13165-13518, and fragments thereof.

60. The AAV particle of claim 59, wherein the capsid is a serotype selected from the group of consisting of SEQ ID NO: 1-1261 and 10928-13139.

61. The AAV particle of claim 60, wherein the regulatory sequence comprises a promoter.

62. The AAV particle of claim 61, wherein the promoter is selected from the group consisting of human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

63. The AAV particle of claim 59, wherein the viral genome is single stranded.

64. The AAV particle of claim 59, wherein the viral genome is self-complementary.

65. The AAV particle of claim 59, wherein at least one region of the viral genome is codon-optimized.

66. The AAV particle of claim 65, wherein the first nucleic acid segment is codon-optimized.

67. The AAV particle of any of claims 59-66, wherein the first nucleic acid segment encodes one or more polypeptides selected from the group consisting of an antibody heavy chain, an antibody light chain, a linker, and combinations thereof.

68. The AAV particle of claim 67, wherein any of the polypeptides encoded by first nucleic acid segment of the payload region is humanized.

69. The AAV particle of claim 67, wherein the linker is selected from one or more of the members of the group given in Table 2.

70. The AAV particle of claim 67, wherein the first nucleic acid segment encodes from 5′ to 3′, an antibody heavy chain, a linker, and an antibody light chain.

71. The AAV particle of claim 67, wherein the first nucleic acid segment encodes from 5′ to 3′, an antibody light chain, a linker, and an antibody heavy chain.

72. The AAV particle of claim 67, wherein the first nucleic acid segment encodes one or more antibody heavy chains.

73. The AAV particle of claim 72, wherein the first nucleic acid segment encodes one or more antibody heavy chain sequences listed in Tables 3-16, and fragments thereof.

74. The AAV particle of claim 67, wherein the first nucleic acid segment encodes one or more antibody light chains.

75. The AAV particle of claim 74, wherein the first nucleic acid segment encodes one or more antibody light chain sequences listed in Tables 3-16 and fragments thereof.

76. The AAV particle of claim 67, wherein the first nucleic acid segment encodes one or more antibody heavy chains and one or more antibody light chains and, optionally one or more linkers.

77. The AAV particle of any of claims 67-76, wherein said linker is selected from the group consisting of Table 2.

78. The AAV particle of claim 59, wherein the first nucleic acid segment encodes an antibody, having at least 95% identity to any of the sequences selected from the group consisting of SEQ ID NO: 1740-10916 and 13165-13518.

79. The AAV particle of claim 59, wherein the viral genome comprises 2 ITR regions.

80. The AAV particle of claim 59, wherein the at least one ITR region is derived from the same parental serotype as the capsid.

81. The AAV particle of claim 59, wherein the at least one ITR region is derived from a different serotype as the capsid.

82. The AAV particle of claim 59, wherein the at least one ITR region is derived from AAV2.

83. The AAV particle of claim 59, wherein the at least one ITR region is 100-150 nucleotides in length.

84. The AAV particle of claim 59, wherein the at least one ITR region is 102 nucleotides in length.

85. The AAV particle of claim 59, wherein the at least one ITR region is 140-142 nucleotides in length.

86. The AAV particle of claim 59, wherein the at least one ITR region is 140 nucleotides in length.

87. The AAV particle of claim 59, wherein the at least one ITR region is 141 nucleotides in length.

88. The AAV particle of claim 59, wherein the at least one ITR region is 142 nucleotides in length.

89. The AAV particle of claim 59, wherein the viral genome further comprises an intron or stuffer sequence.

90. The AAV particle of claim 59, wherein the payload region of the viral genome comprises a second nucleic acid segment, said second nucleic acid segment encoding an aptamer, siRNA, saRNA, ribozyme, microRNA, mRNA or combination thereof.

91. The AAV particle of claim 90, wherein the second nucleic acid segment encodes an siRNA and said siRNA is designed to target the mRNA that encodes the target of the antibody encoded by the first nucleic acid segment.

92. The AAV particle of claim 90, wherein the second nucleic acid segment encodes a microRNA and said microRNA is selected to target the mRNA that encodes the target of the antibody encoded by the first nucleic acid segment.

93. The AAV particle of claim 90, wherein the second nucleic acid segment encodes an mRNA and said mRNA encodes one or more peptides inhibitors of the same target of the antibody encoded by the first nucleic acid segment.

94. The AAV particle of claim 59 or 90, wherein the payload region of the viral genome comprises a third nucleic acid segment.

95. The AAV particle of claim 94, wherein the third nucleic acid segment encodes a nuclear export signal.

96. The AAV particle of claim 94, wherein the third nucleic acid segment encodes a polynucleotide or polypeptide which acts as a regulator of expression of the viral genome in which it is encoded.

97. The AAV particle of claim 94, wherein the third nucleic acid segment encodes a polynucleotide or polypeptide which acts as a regulator of expression of the payload region of the viral genome in which it is encoded.

98. The AAV particle of claim 94, wherein the third nucleic acid segment encodes a polynucleotide or polypeptide which acts as a regulator of expression of the first nucleic acid segment of the payload region of the viral genome in which it is encoded.

99. An AAV particle comprising a capsid and a viral genome, said viral genome comprising at least one inverted terminal repeat (ITR) region and a payload region comprising a regulatory sequence operably linked to at least a first nucleic acid segment, said first nucleic acid segment encoding a bispecific antibody derived from SEQ ID NO: 1740-10916 and 13165-13518 or portions or fragments thereof.

100. The AAV particle of claim 99, wherein the bispecific antibody comprises a light and a heavy chain selected from two different starting antibodies sequences listed in Tables 3-16.

101. A pharmaceutical composition comprising an AAV particle of any of the preceding claims in a pharmaceutically acceptable excipient.

102. The pharmaceutical composition of claim 101, wherein the pharmaceutically acceptable excipient is saline.

103. The pharmaceutical composition of claim 101, wherein the pharmaceutically acceptable excipient is 0.001% pluronic in saline.

104. A method of producing an antibody in a subject in need thereof, comprising administering to said subject the AAV particle of any of claims 1-100 or the pharmaceutical composition of any of claims 101-103.

105. The method of claim 104, wherein the level or amount of the antibody in the target cell or tissue after administration to the subject is from about 0.001 ug/mL to 100 mg/mL.

106. The method of claim 104, wherein the antibody is encoded by a single first nucleic acid segment of a viral genome within said AAV particle.

107. The method of claim 104, wherein the antibody is encoded by two different viral genomes, said two different viral genomes packaged in separate capsids.

108. A method of expressing an antibody in a cell or tissue comprising administering the AAV particle of any of claims 1-100 or the pharmaceutical composition of any of claims 101-103 via a delivery route selected from the group consisting of enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into the substance of a tissue, e.g., brain tissue), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

109. The method of claim 108, wherein the delivery route is intramuscular.

110. The method of claim 109, wherein the intramuscular administration is to at least one limb.

111. The method of claim 108, wherein the delivery route is intravascular.

112. The method of claim 108, wherein the delivery route is intrathecal.

113. The method of claim 108, wherein the delivery route is intracerebroventricular.

114. The method of claim 108, wherein the delivery route is intraparenchymal.

115. The method of claim 108, wherein the AAV particle is encapsulated in a nanoparticle.

116. The method of claim 108, wherein the AAV particle is delivered by a device.

117. The method of claim 116, wherein the device is a gene gun.

118. A method of preventing a disease or disorder in a subject comprising administering to said subject the pharmaceutical composition of any of claims 101-103.

119. The method of claim 118, wherein the administration is at a prophylactically effective dose.

120. The method of claim 119, wherein the dose is from about 1 ug/mL to about 500 ug/mL of expressed polypeptide or 1x10e4 to 1x10e16 VG/mL from the pharmaceutical composition.

121. The method of claim 120, wherein the pharmaceutical composition is administered once.

122. The method of claim 120, wherein the pharmaceutical composition is administered more than once.

123. The method of claim 120, wherein the pharmaceutical composition is administered daily, weekly, monthly or yearly.

124. The method of claim 120, wherein the pharmaceutical composition is co-administered as part of a combination therapy.

125. A method of treating a disease or disorder in a subject in need thereof comprising administering to said subject the pharmaceutical composition of any of claims 101-103.

126. The method of claim 125, wherein said disease or disorder is selected from the group consisting of diseases caused by John Cunningham Virus (JCV), influenza, hepatitis A, hepatitis B, hepatitis D, hepatitis E, respiratory syncytial virus (RSV), herpes simplex virus 1, herpes simplex virus 2, human cytomegalovirus, Epstein-Barr virus, Varicella zoster virus, Coronavirus, Poxvirus, Enterovirus 71, rubella virus, human papilloma virus, Pseudomonas Aeruginosa, Streptococcus bacteria, Stapylococcus bacteria, Clostridium Tetani, Bordetella, Mycobacterium, Francisella Tularensis, Toxoplasma gondii, Candida yeast, ricin, Bacillus anthracis, shiga toxin, shiga-like toxin, botulinum toxins, chikungunya virus, dengue virus, trypasnosoma cruzi, rabies virus, Plasmodium falciparum, ebola virus, Marburg virus, West Nile virus, Yellow Fever virus, Japanese encephalitis virus, St. Louis encephalitis virus, rotavirus, Norwalk virus, Campylobacter jejuni, Clostridium difficile, Entamoeba histolytica, Helicobacter pylori, and Enterotoxin B, Parkinson's Disease, Dementia with Lewy Bodies, multiple system atrophy, decreased muscle mass, decreased muscle strength, decreased muscle function, spinal muscular atrophy, Alzheimer's Disease, Huntington's Disease, multiple sclerosis, amyotrophic lateral sclerosis, stroke, migraine, pain, neuropathies, psychiatric disorders, cancer, ocular diseases, systemic diseases of the blood, systemic diseases of the heart, systemic diseases of the bone, immune system, autoimmune disease, inflammation disorders and inflammation.
